(19) United States
(12) Patent Application Publication
(10) Pub. No.: US 2025/0195625 A1
(43) Pub. Date: Jun. 19, 2025

(54) CXCL-MODULATING COMPOSITIONS AND METHODS

(71) Applicant: FLAGSHIP PIONEERING INNOVATIONS V, INC., Cambridge, MA (US)

(72) Inventors: Laura Anh Nguyen, Medford, MA (US); Jeremiah Dale Farelli, Marblehead, MA (US); Joseph Newman, Sudbury, MA (US); Lauren Marie Beech, Plymouth, MA (US); Mithilesh K. Jha, Sharon, MA (US); Charles O'Donnell, Winchester, MA (US); Houda Belaghzal, Cambridge, MA (US)

(21) Appl. No.: 18/852,300

(22) PCT Filed: Mar. 29, 2023
(86) PCT No.: PCT/US2023/065108

§ 371 Date: Sep. 27, 2024

Related U.S. Application Data
(60) Provisional application No. 63/325,524, filed on Mar. 30, 2022.
(60) Provisional application No. 63/379,849, filed on Oct. 17, 2022.
(60) Provisional application No. 63/478,855, filed on Jan. 6, 2023.

(51) Int. Cl. A61K 38/46; A61K 31/7105; A61P 11/00; A61P 35/00; C12N 9/22; C12N 15/11

(52) U.S. Cl. CPC A61K 38/465; A61K 31/7105; A61P 11/00; A61P 35/00; C12N 9/22; C12N 15/11; C12N 2310/20

(57) ABSTRACT

The present disclosure relates to expression repressors decreasing expression of a target plurality of genes in a cell. In some embodiments, the target plurality of genes comprises CXCL1, CXCL2, CXCL3, CXCL4, CXCL5, CXCL6, CXCL7, and IL-8. In some embodiments, the expression repressor targets the E1 cRE of the CXCL locus. In some embodiments, the expression repressor targets the E2 cRE of the CXCL locus.

### CROSS-REFERENCE TO RELATED APPLICATIONS

This application claims priority to U.S. Provisional Application 63/325,524 filed on Mar. 30, 2022, U.S. Provisional Application 63/379,849 filed on Oct. 17, 2022, and U.S. Provisional Application 63/478,855 filed on Jan. 6, 2023, the entire contents of which are hereby incorporated by reference.

### SEQUENCE LISTING

The instant application contains a Sequence Listing which has been submitted electronically in XML format and is hereby incorporated by reference in its entirety. Said XML copy, created on Mar. 28, 2023, is named 02057-7032WO_SL and is 661,150 bytes in size.

### BACKGROUND

Mis-regulation of gene expression is the underlying cause of many diseases (e.g., in mammals, e.g., humans). A number of diseases and conditions are associated with pluralities of related genes. There is a need for novel tools, systems, and methods to alter, e.g., decrease, expression of pluralities of associated genes.

### SUMMARY

The disclosure provides, among other things, expression repressors or systems comprising expression repressors that may be used to modulate, e.g., decrease, expression of a one or more target genes, e.g., one or more CXCL genes, that are within a CXCL locus comprising a cis-acting regulatory element.

In one aspect, the disclosure provides an expression repressor comprising:
a first targeting moiety that binds a target site comprising a cis-acting regulatory element, e.g., an enhancer (e.g., an enhancer for a CXCL gene); and
a first effector moiety.

In one aspect, the disclosure provides an expression repressor comprising:
a first targeting moiety that binds to a target site, wherein the target site is within a cis-acting regulatory element of a CXCL locus, and
optionally, a first effector moiety,
wherein the expression repressor is capable of decreasing expression of a CXCL gene.

In one aspect, the disclosure provides an expression repressor comprising:
a first targeting moiety that binds a target site comprising an IL-8 promoter; and
a first effector moiety.

In one aspect, the disclosure provides an expression repressor comprising:
a first targeting moiety that binds to a target site, wherein the target site is within an E1 cis-acting regulatory element of a CXCL locus or an E2 cis-acting regulatory element of a CXCL locus, and
optionally, a first effector moiety,
wherein the expression repressor is capable of decreasing expression of a CXCL gene.

In some embodiments, the target site is within genomic coordinates chr4: 74591400-74593000 or chr4:74982639-74983600 (based on hg19 human genome reference assembly).

In one aspect, the disclosure provides an expression repressor comprising: a first targeting moiety that binds to a target site within genomic coordinates chr4: 74591400-74593000 or chr4:74982639-74983600 (based on hg19 human genome reference assembly), and
optionally, a first effector moiety,
wherein the expression repressor is capable of decreasing expression of a CXCL gene.

In some embodiments, the target site is chosen from:
a) GRCh37: chr4:74591777-74591797;
b) GRCh37: chr4:74591834-74591854;
c) GRCh37: chr4:74591896-74591916;
d) GRCh37: chr4:74592082-74592102;
e) GRCh37: chr4:74592107-74592127;
f) GRCh37: chr4:74592156-74592176;
g) GRCh37: chr4:74592210-74592230;
h) GRCh37: chr4:74592057-74592077;
i) GRCh37: chr4:74591977-74591997;
j) GRCh37: chr4:74591856-74591876;
k) GRCh37: chr4:74591768-74591790;
l) GRCh37: chr4:74591844-74591866;
m) GRCh37: chr4:74591892-74591914;
n) GRCh37: chr4:74592088-74592110;
o) GRCh37: chr4:74982748-74982770;
p) GRCh37: chr4:74982841-74982863;
q) GRCh37: chr4:74982882-74982904;
r) GRCh37: chr4:74982960-74982982;
s) GRCh37: chr4:74983108-74983130; and
t) GRCh37: chr4:74983181-74983203.

In certain embodiments, the first targeting moiety binds within 500, 300, 200, 100, or 50 nucleotides upstream or downstream of a target site chosen from:
a) GRCh37: chr4:74591777-74591797;
b) GRCh37: chr4:74591834-74591854;
c) GRCh37: chr4:74591896-74591916;
d) GRCh37: chr4:74592082-74592102;
e) GRCh37: chr4:74592107-74592127;
f) GRCh37: chr4:74592156-74592176;
g) GRCh37: chr4:74592210-74592230;
h) GRCh37: chr4:74592057-74592077;
i) GRCh37: chr4:74591977-74591997;
j) GRCh37: chr4:74591856-74591876;
k) GRCh37: chr4:74591768-74591790;
l) GRCh37: chr4:74591844-74591866;
m) GRCh37: chr4:74591892-74591914;
n) GRCh37: chr4:74592088-74592110;
o) GRCh37: chr4:74982748-74982770;
p) GRCh37: chr4:74982841-74982863;
k) GRCh37: chr4:74982882-74982904;
r) GRCh37: chr4:74982960-74982982;
s) GRCh37: chr4:74983108-74983130; and
t) GRCh37: chr4:74983181-74983203.

In one aspect, the disclosure provides an expression repressor comprising:
a first targeting moiety that binds a target site comprising at least 15, 16, 17, 18, 19, 20, 21, 22, 23, 24, 25, 26, 27, 28, or 29, nucleotides of the sequence of any one of SEQ ID NOs: 163 or 164, or a sequence having at least 80%, 85%, 90%, 95%, 96%, 97%, 98%, 99% identity thereto, or a sequence with no more than 20, 19, 18, 17, 16, 15, 14, 13, 12, 11, 10, 9, 8, 7, 6, 5, 4, 3, 2, or 1 positions of difference thereto, and
optionally, a first effector moiety,
wherein the expression repressor is capable of decreasing expression of a CXCL gene.

In one aspect, the disclosure provides an expression repressor comprising:
a first targeting moiety that binds to a target site, wherein the target site is within an IL-8 promoter, and
optionally, a first effector moiety,
   wherein the expression repressor is capable of decreasing expression of IL-8.

In some embodiments, the target site (e.g., target site within the IL8 promoter) is within genomic coordinates chr4:74606112-74606462 (hg19). In some embodiments, the target site (e.g., target site within the IL8 promoter) is located within 1 kb from chr:74606112-74606462 (e.g., chr4:74606112-74606662, chr4:74606112-74606862, chr4:74606112-74607062, chr4:74606112-74607262, chr4:74606112-74607462, chr4:74605912-74606462, chr4:74605712-74606462, chr4:74605512-74606462, chr4:74605312-74606462, chr4:74605112-74606462, chr4:74605912-74606662, chr4:74605912-74606862, chr4:74605912-74607062, chr4:74605912-74607262, chr4:74605912-74607462, chr4:74605712-74606662, chr4:74605712-74606862, chr4:74605712-74607062, chr4:74605712-74607262, chr4:74605712-74607462, chr4:74605512-74606662, chr4:74605512-74606862, chr4:74605512-74607062, chr4:74605512-74607262, chr4:74605512-74607462, chr4:74605312-74606662, chr4:74605312-74606862, chr4:74605312-74607062, chr4:74605312-74607262, chr4:74605312-74607462, chr4:74605112-74606662, chr4:74605112-74606862, chr4:74605112-74607062, chr4:74605112-74607262, or chr4:74605112-74607462). In certain embodiments, the target site (e.g., target site within the IL8 promoter) is located 500 bp upstream from the transcription start site. In certain embodiments, the target site (e.g., target site within the IL8 promoter) is located at chr4:74605723-74606223. In some embodiments, the target site (e.g., target site within the IL8 promoter) is located at chr4:74605723-74606426, chr4:74605723-74606626, chr4:74605723-74606826, chr4:74605723-74607026, chr4:74605723-74607226, chr4:74605523-74606226, chr4:74605323-74606226, chr4:74605123-74606226, chr4:74604923-74606226, chr4:74604723-74606226, chr4:74605523-74606426, chr4:74605523-74606626, chr4:74605523-74606826, chr4:74605523-74607026, chr4:74605523-74607226, chr4:74605323-74606426, chr4:74605323-74606626, chr4:74605323-74606826, chr4:74605323-74607026, chr4:74605323-74607226, chr4:74605123-74606426, chr4:74605123-74606626, chr4:74605123-74606826, chr4:74605123-74607026, chr4:74605123-74607226, chr4:74604923-74606426, chr4:74604923-74606626, chr4:74604923-74606826, chr4:74604923-74607026, chr4:74604923-74607226, chr4:74604723-74606426, chr4:74604723-74606626, chr4:74604723-74606826, chr4:74604723-74607026, or chr4:74604723-74607226. In some embodiments, the target site (e.g., target site within the IL8 promoter) is located 1000 bp upstream from the transcription start site. In certain embodiments, the target site (e.g., target site within the IL8 promoter) is located at chr4:74605223-74606223. In some embodiments, the target site (e.g., target site within the IL8 promoter) is located at chr4:74605226-74606426, chr4:74605226-74606626, chr4:74605226-74606826, chr4:74605226-74607026, chr4:74605226-74607226, chr4:74605026-74606226, chr4:74604826-74606226, chr4:74604626-74606226, chr4:74604426-74606226, chr4:74604226-74606226, chr4:74605026-74606426, chr4:74605026-74606626, chr4:74605026-74606826, chr4:74605026-74607026, chr4:74605026-74607226, chr4:74604826-74606426, chr4:74604826-74606626, chr4:74604826-74606826, chr4:74604826-74607026, chr4:74604826-74607226, chr4:74604626-74606426, chr4:74604626-74606626, chr4:74604626-74606826, chr4:74604626-74607026, chr4:74604626-74607226, chr4:74604426-74606426, chr4:74604426-74606626, chr4:74604426-74606826, chr4:74604426-74607026, chr4:74604426-74607226, chr4:74604226-74606426, chr4:74604226-74606626, chr4:74604226-74606826, chr4:74604226-74607026, or chr4:74604226-74607226.

In one aspect, the disclosure provides an expression repressor comprising:
a first targeting moiety that binds to a target site within genomic coordinates GRCh37: chr4:74606162-74606184, GRCh37: chr4: 74605723-74606223, or GRCh37: chr4: 74605223-74606223 (based on hg19 human genome reference assembly); and
optionally, a first effector moiety,
   wherein the expression repressor is capable of decreasing expression of IL-8.

In some embodiments, the expression repressor binds to a target site is chosen from:
i) GRCh37: chr4:74605780-74605800;
ii) GRCh37: chr4:74605961-74605981;
iii) GRCh37: chr4:74606122-74606142;
iv) GRCh37: chr4:74605955-74605975;
v) GRCh37: chr4:74605842-74605862;
vi) GRCh37: chr4:74606145-74606165;
vii) GRCh37: chr4:74606039-74606056;
viii) GRCh37: chr4:74606113-74606130;
ix) GRCh37: chr4:74606137-74606154;
x) GRCh37: chr4:74606150-74606167;
xi) GRCh37: chr4:74591882-74591899;
xii) GRCh37: chr4:74591923-74591940;
xiii) GRCh37: chr4:74591897-74591914; and
xiv) GRCh37: chr4:74591873-74591890.

In certain embodiments, the first targeting moiety binds within 500, 300, 200, 100, or 50 nucleotides upstream or downstream of a target site chosen from:
i) GRCh37: chr4:74605780-74605800;
ii) GRCh37: chr4:74605961-74605981;
iii) GRCh37: chr4:74606122-74606142;
iv) GRCh37: chr4:74605955-74605975;
v) GRCh37: chr4:74605842-74605862;
vi) GRCh37: chr4:74606145-74606165;
vii) GRCh37: chr4:74606039-74606056;
viii) GRCh37: chr4:74606113-74606130;
ix) GRCh37: chr4:74606137-74606154;
x) GRCh37: chr4:74606150-74606167;
xi) GRCh37: chr4:74591882-74591899;
xii) GRCh37: chr4:74591923-74591940;
xiii) GRCh37: chr4:74591897-74591914; and
xiv) GRCh37: chr4:74591873-74591890.

In some embodiments, the first effector moiety comprises an effector described herein, e.g., KRAB, MQ1, DNMT1, DNMT3A1, DNMT3A2, DNMT3B1, DNMT3B2, DNMT3B3, DNMT3B4, DNMT3B5, DNMT3B6, DNMT3L, EZH2, HDAC8, MeCP2, HP1, RBBP4, REST, FOG1, SUZ12, SETDB1, SETDB2, EHMT2 (i.e., G9A), EHMT1 (i.e., GLP), SUV39H1, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, EZH1, SUV39H2, SETD8, SUV420H1, SUV420H2 or DNMT3, or a functional variant or fragment of any thereof.

In certain embodiments, the first effector moiety is linked to the targeting moiety via a linker. In some embodiments the linker is a peptide linker. In some embodiments, the linker may be between 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 amino acids in length, or greater than or equal to 2, 5, 10, 15, 20, 25, or 30 amino acids in length (and optionally up to 50, 40, 30, 25, 20, 15, 10, or 5 amino acids in length).

In some embodiments, the first effector moiety is C-terminal of the targeting moiety.

In certain embodiments, the first effector moiety is N-terminal of the targeting moiety.

In some embodiments, the first effector moiety is encoded by a nucleotide sequence chosen from any of SEQ ID NOs: 10, 14, 16, 18, 66, 68, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In certain embodiments, the first effector moiety comprises an amino acid sequence according to any of SEQ ID NOs: 11, 12, 13, 15, 17, 19, 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, the first effector moiety is MQ1 or a functional variant or fragment thereof, e.g., wherein the first effector moiety comprises an amino acid sequence of SEQ ID NO: 11 or 12 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the first effector moiety is C-terminal of the first targeting moiety.

In certain embodiments, the first effector moiety is KRAB, or a functional variant or fragment thereof, e.g., wherein the first effector moiety comprises an amino acid sequence of SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the first effector moiety is C-terminal of the first targeting moiety.

In some embodiments, the first effector moiety is DNMT3a/3L, or a functional variant or fragment thereof, e.g., wherein the first effector moiety comprises an amino acid sequence of SEQ ID NO: 15 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the first effector moiety is C-terminal of the first targeting moiety.

In certain embodiments, the first effector moiety is EZH2, or a functional variant or fragment thereof, e.g., wherein the first effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, the first effector moiety is HDAC8, or a functional variant or fragment thereof, e.g., wherein the first effector moiety comprises an amino acid sequence of SEQ ID NO: 19 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the first effector moiety is C-terminal of the first targeting moiety.

In certain embodiments, the first effector moiety is G9A, or a functional variant or fragment thereof, e.g., wherein the first effector moiety comprises an amino acid sequence of SEQ ID NO: 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the first effector moiety is N-terminal of the first targeting moiety.

In some embodiments, the effector moiety comprises a DNA methyltransferase, e.g., MQ1 or a fragment or variant thereof.

In certain embodiments, the effector moiety comprises a transcription repressor, e.g., comprises KRAB or a fragment or variant thereof.

In some embodiments, the target site has a length of 15-20, 20-25, 25-30, or 30-35 nucleotides.

In some embodiments, the first targeting moiety comprises a zinc finger domain.

In certain embodiments, the zinc finger domain comprises 1, 2, 3, 4, 5, 6, 7, 8, 9, or 10 zinc fingers (and optionally no more than 11, 10, 9, 8, 7, 6, or 5 zinc fingers).

In some embodiments, the zinc finger domain comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 zinc fingers.

In some embodiments, the zinc finger domain comprises 3, 7, or 9 zinc fingers. In some embodiments, the zinc finger domain targets a site comprising 21 nucleotides.

In certain embodiments, the first targeting moiety comprises a CRISPR-Cas domain.

In certain embodiments, the expression repressor described herein is capable of decreasing expression of a plurality of CXCL genes (e.g., 2, 3, 4, 5, 6, 7, or 8 CXCL genes). In certain embodiments, the expression repressor described herein is capable of decreasing expression of one or more of (e.g., 2, 3, 4, 5, 6, 7, or 8 of) CXCL1, CXCL2, CXCL3, CXCL4, CXCL5, CXCL6, CXCL7, or IL-8.

In certain embodiments, the first effector moiety is a durable effector moiety or a transient effector moiety.

In some embodiments, the first targeting moiety comprises a zinc finger domain, and the first effector moiety comprises a transcription repressor, e.g., KRAB or a fragment or variant thereof.

In some embodiments, the first targeting moiety comprises a zinc finger domain, and the first effector moiety comprises an epigenetic modifying moiety, e.g., a DNA methyltransferase, e.g., MQ1 or a fragment or variant thereof.

In some embodiments, the expression repressor comprises an amino acid sequence of any one of SEQ ID NOs: 152-161, or a sequence having at least 80%, 85%, 90%, 95%, 96%, 97%, 98%, 99% identity thereto, or a sequence with no more than 20, 19, 18, 17, 16, 15, 14, 13, 12, 11, 10, 9, 8, 7, 6, 5, 4, 3, 2, or 1 positions of difference thereto.

In some embodiments, the expression repressor described herein: (i) comprises one or more nuclear localization signal sequences (NLS), or (ii) does not comprise an NLS.

In some embodiments, the expression repressor described herein comprises a first NLS at the N terminus, e.g., wherein the first NLS has a sequence of SEQ ID NO: 63 or 64.

In some embodiments, the expression repressor described herein comprises an NLS, e.g., a second NLS, at the C terminus, e.g., having a sequence of SEQ ID NO: 63 or 64.

In some embodiments, the first and the second NLS have the same sequence. In certain embodiments, the first and the second NLS have different sequences.

In certain embodiments, binding of the expression repressor to the target site increases methylation at a site in the CXCL locus, e.g., increases methylation at the E1 cis-acting regulatory element of the CXCL locus or the E2 cis-acting regulatory element of the CXCL locus.

In one aspect, the disclosure provides a system comprising:
a) a first expression repressor according to any of the previous embodiments, and
b) a second expression repressor, e.g., a second expression repressor that decreases expression of a CXCL gene.

In some embodiments, the second expression repressor comprises:
a second targeting moiety that binds to a second target site within the CXCL locus, and
optionally, a second effector moiety.

In certain embodiments, second expression repressor binds to the E1 cis-acting regulatory element of the CXCL locus, E2 cis-acting regulatory element of the CXCL locus, or IL8 promoter.

In certain embodiments, the second target site is within coordinates GRCh37: chr4:74606162-74606184, GRCh37: chr4: 74605723-74606223, or GRCh37: chr4: 74605223-74606223. In some embodiments, the second target site is within coordinates:
a) chr4:74606112-74606462, chr4:74606112-74606662, chr4:74606112-74606862, chr4:74606112-74607062, chr4:74606112-74607262, chr4:74606112-74607462, chr4:74605912-74606462, chr4:74605712-74606462, chr4:74605512-74606462, chr4:74605312-74606462, chr4:74605112-74606462, chr4:74605912-74606662, chr4:74605912-74606862, chr4:74605912-74607062, chr4:74605912-74607262, chr4:74605912-74607462, chr4:74605712-74606662, chr4:74605712-74606862, chr4:74605712-74607062, chr4:74605712-74607262, chr4:74605712-74607462, chr4:74605512-74606662, chr4:74605512-74606862, chr4:74605512-74607062, chr4:74605512-74607262, chr4:74605512-74607462, chr4:74605312-74606662, chr4:74605312-74606862, chr4:74605312-74607062, chr4:74605312-74607262, chr4:74605312-74607462, chr4:74605112-74606662, chr4:74605112-74606862, chr4:74605112-74607062, chr4:74605112-74607262, or chr4:74605112-74607462;
b) chr4:74605723-74606223, chr4:74605723-74606426, chr4:74605723-74606626, chr4:74605723-74606826, chr4:74605723-74607026, chr4:74605723-74607226, chr4:74605523-74606226, chr4:74605323-74606226, chr4:74605123-74606226, chr4:74604923-74606226, chr4:74604723-74606226, chr4:74605523-74606426, chr4:74605523-74606626, chr4:74605523-74606826, chr4:74605523-74607026, chr4:74605523-74607226, chr4:74605323-74606426, chr4:74605323-74606626, chr4:74605323-74606826, chr4:74605323-74607026, chr4:74605323-74607226, chr4:74605123-74606426, chr4:74605123-74606626, chr4:74605123-74606826, chr4:74605123-74607026, chr4:74605123-74607226, chr4:74604923-74606426, chr4:74604923-74606626, chr4:74604923-74606826, chr4:74604923-74607026, chr4:74604923-74607226, chr4:74604723-74606426, chr4:74604723-74606626, chr4:74604723-74606826, chr4:74604723-74607026, or chr4:74604723-74607226; or
c) chr4:74605223-74606223, chr4:74605226-74606426, chr4:74605226-74606626, chr4:74605226-74606826, chr4:74605226-74607026, chr4:74605226-74607226, chr4:74605026-74606226, chr4:74604826-74606226, chr4:74604626-74606226, chr4:74604426-74606226, chr4:74604226-74606226, chr4:74605026-74606426, chr4:74605026-74606626, chr4:74605026-74606826, chr4:74605026-74607026, chr4:74605026-74607226, chr4:74604826-74606426, chr4:74604826-74606626, chr4:74604826-74606826, chr4:74604826-74607026, chr4:74604826-74607226, chr4:74604626-74606426, chr4:74604626-74606626, chr4:74604626-74606826, chr4:74604626-74607026, chr4:74604626-74607226, chr4:74604426-74606426, chr4:74604426-74606626, chr4:74604426-74606826, chr4:74604426-74607026, chr4:74604426-74607226, chr4:74604226-74606426, chr4:74604226-74606626, chr4:74604226-74606826, chr4:74604226-74607026, or chr4:74604226-74607226.

In certain embodiments, the second target site is within GRCh37: chr4:74606162-74606184.

In some embodiments, the second target site is chosen from:
i) GRCh37: chr4:74605780-74605800;
ii) GRCh37: chr4:74605961-74605981;
iii) GRCh37: chr4:74606122-74606142;
iv) GRCh37: chr4:74605955-74605975;
v) GRCh37: chr4:74605842-74605862;
vi) GRCh37: chr4:74606145-74606165;
vii) GRCh37: chr4:74606039-74606056;
viii) GRCh37: chr4:74606113-74606130;
ix) GRCh37: chr4:74606137-74606154;
x) GRCh37: chr4:74606150-74606167;
xi) GRCh37: chr4:74591882-74591899;
xii) GRCh37: chr4:74591923-74591940;
xiii) GRCh37: chr4:74591897-74591914; and
xiv) GRCh37: chr4:74591873-74591890.

In certain embodiments, the second target site is located within 500, 300, 200, 100, or 50 nucleotides upstream or downstream of a target site chosen from:
i) GRCh37: chr4:74605780-74605800;
ii) GRCh37: chr4:74605961-74605981;
iii) GRCh37: chr4:74606122-74606142;
iv) GRCh37: chr4:74605955-74605975;
v) GRCh37: chr4:74605842-74605862;
vi) GRCh37: chr4:74606145-74606165;
vii) GRCh37: chr4:74606039-74606056;
viii) GRCh37: chr4:74606113-74606130;
ix) GRCh37: chr4:74606137-74606154;
x) GRCh37: chr4:74606150-74606167;
xi) GRCh37: chr4:74591882-74591899;
xii) GRCh37: chr4:74591923-74591940;
xiii) GRCh37: chr4:74591897-74591914; and
xiv) GRCh37: chr4:74591873-74591890.

In certain embodiments, the second targeting moiety is a clustered regulatory interspaced short palindromic repeat (CRISPR) Cas domain.

In one aspect, the disclosure provides a nucleic acid encoding an expression repressor described herein.

In one aspect, the disclosure provides a nucleic acid encoding: a first expression repressor of any described herein and a second expression repressor, e.g., a second expression repressor that decreases expression of a CXCL gene, e.g., an expression repressor of the system of any of the previous aspects of embodiments.

In one aspect, the disclosure provides a nucleic acid system comprising:
a) a first nucleic acid encoding a first expression repressor as described herein, and
b) a second nucleic acid encoding a second expression repressor, e.g., a second expression repressor that decreases expression of a CXCL gene, e.g., an expression repressor of the system of any of the previous aspects of embodiments.

In some embodiments, nucleic acid or nucleic acid system comprises a region encoding the first targeting moiety, wherein the region encoding the first targeting moiety comprises a nucleotide sequence of any one of SEQ ID NO: 122-131, or a sequence having at least 80%, 85%, 90%, 95%, 96%, 97%, 98%, or 99% identity thereto.

In some embodiments, nucleic acid or nucleic acid system comprises a region encoding the first targeting moiety, wherein the region encoding the first targeting moiety comprises a nucleotide sequence of any one of SEQ ID NOs: 194-199, 248-253, or 276-291, or a sequence having at least 80%, 85%, 90%, 95%, 96%, 97%, 98%, or 99% identity thereto.

In some embodiments, the nucleic acid or nucleic acid system comprises a region encoding the first effector moiety, wherein the region encoding the first effector moiety comprises a nucleotide sequence of any one of SEQ ID NO: 10, 14, 16, 18, 66, 68, or a sequence having at least 80%, 85%, 90%, 95%, 96%, 97%, 98%, or 99% identity thereto.

In some embodiments, the nucleic acid or nucleic acid system further comprises a region encoding an NLS. In certain embodiments, the region encoding the NLS comprises a nucleotide sequence of SEQ ID NO: 63 or 64, or a sequence having at least 80%, 85%, 90%, 95%, 96%, 97%, 98%, or 99% identity thereto.

In certain embodiments, nucleic acid or nucleic acid system comprises DNA or RNA (e.g., mRNA).

In one aspect, the disclosure provides a vector comprising the nucleic acid or nucleic acid system of any one of the previous aspects or embodiments.

In one aspect, the disclosure provides a pharmaceutical composition comprising the expression repressor, nucleic acid, or nucleic acid system of any of the preceding aspects or embodiments.

In some embodiments, the pharmaceutical composition comprises an LNP, e.g., wherein the nucleic acid or nucleic acid system is formulated as an LNP.

In one aspect, the disclosure provides a human cell comprising: an expression repressor as described herein, a nucleic acid or nucleic acid system as described herein, or a vector as described herein.

In one aspect, the disclosure provides a human cell having decreased expression of a CXCL gene, wherein the cell was produced by a method comprising contacting the cell with an expression repressor of any of the previous aspects or embodiments, a nucleic acid or nucleic acid system of any of the previous aspects or embodiments, or a vector of any of the previous aspects or embodiments.

In some embodiments, the human cell has decreased expression of a first and a second CXCL gene. In certain embodiments, the human cell has decreased expression of a third CXCL gene. In certain embodiments, the human cell has decreased expression of a fourth CXCL gene. In some embodiments, the human cell has decreased expression of a fifth CXCL gene. In certain embodiments, the human cell has decreased expression of a sixth CXCL gene. In some embodiments, the human cell has decreased expression of a seventh CXCL gene. In some embodiments, the human cell has decreased expression of an eighth CXCL gene. In some embodiments, the human cell has decreased expression of one or more of (e.g., 2, 3, 4, 5, 6, 7, or 8 of) CXCL1, CXCL2, CXCL3, CXCL4, CXCL5, CXCL6, CXCL7, or TL-8. In some embodiments, the human cell has decreased expression of one or more of CXCL1, CXCL2, CXCL3, and IL8.

In one aspect, the disclosure provides a method of decreasing expression of one or more CXCL genes in a cell, comprising contacting the cell with an expression repressor, a system, a nucleic acid or nucleic acid system, or a vector of any one of the previous aspects or embodiments.

In one aspect, the disclosure provides a method of decreasing expression of IL-8 in a cell, the method comprising contacting the cell with an expression repressor, a system, a nucleic acid or nucleic acid system described herein.

In one aspect, the disclosure provides a method of decreasing expression of one or more CXCL genes in a cell, comprising contacting the cell with an expression repressor, or a nucleic acid comprising a sequence encoding the expression repressor, wherein the expression repressor comprises:
a first targeting moiety that binds to a target site, wherein the target site is within an E1 cis-acting regulatory element of a CXCL locus or an E2 cis-acting regulatory element of a CXCL locus, and
optionally, a first effector moiety, thereby decreasing expression of a CXCL gene.

In some embodiments, the target site is within genomic coordinates chr4: 74591400-74593000 or chr4:74982639-74983600 (based on hg19 human genome reference assembly).

In certain embodiments, expression of one or more of (e.g., 2, 3, 4, 5, 6, 7, or 8 of) CXCL1, CXCL2, CXCL3, CXCL4, CXCL5, CXCL6, CXCL7, or IL-8 is decreased.

In some embodiments, expression is decreased for at least 1, 2, 3, 4, 5, 6, 7, 10, or 14 days, or at least 1, 2, 3, 4, or 5 weeks.

In some embodiments, the cell is a cell of a subject having an inflammatory disease, e.g., an immune mediated inflammatory disease. In certain embodiments, the inflammatory disease is an autoimmune disorder, e.g., rheumatoid arthritis.

In some embodiments, the inflammatory disease is associated with a pathogenic infection, e.g., viral infection, e.g., SARS-CoV2 infection.

In certain embodiments, the inflammatory disease is associated with a superinfection, e.g., infection caused by two or more pathogenic agents, e.g., by a virus and a bacterium, (e.g., by SARS-CoV2 and Streptococcus pneumoni), e.g., by a virus and a fungus, (e.g., by SARS-CoV2 and mucormycosis).

In some embodiments, the cell is a cell of a subject having rheumatoid arthritis, inflammatory, arthritis, gout, asthma, neutrophilic asthma, neutrophilic dermatosis, paw edema, acute respiratory disease syndrome (ARDS), COVID-19, psoriasis, inflammatory bowel disease, infection (e.g., by a pathogen, e.g., a bacteria, a viruses, or a fungus), external injury (e.g., scrapes or foreign objects), effects of radiation or chemical injury, osteoarthritis, osteoarthritic joint pain, joint pain, inflammatory pain, acute pain, chronic pain, cystitis, bronchitis, dermatitis, dermatosis, cardiovascular disease, neurodegenerative disease, liver disease, lung disease, kidney disease, pain, swelling, stiffness, tenderness, redness, warmth, or elevated biomarkers related to disease states (e.g., cytokines, chemokines, growth factors, immune receptors, infection markers, or inflammatory markers).

In some embodiments, the cell is a cell of a subject having rheumatoid arthritis, psoriasis, or inflammatory bowel disease.

In certain embodiments, the cell is a cell of a subject having rheumatoid arthritis, gout, neutrophilic asthma, neutrophilic dermatosis, acute respiratory disease syndrome (ARDS), or COVID-19.

In some embodiments, the cell is a cell of a subject having cancer.

In certain embodiments, the cancer is lung cancer (e.g., non-small cell lung cancer), breast cancer, hepatocellular carcinoma (HCC), prostate cancer, colon cancer, skin cancer, cervical cancer, ovarian cancer, uterine endometrioid carcinoma, endometrial cancer, mature B-cell lymphoma, bladder cancer, esophagogastric cancer, esophageal adenocarcinoma, bone cancer, melanoma, hepatobiliary cancer, thyroid cancer, mature B-cell neoplasms, glioma, head-neck squamous cell carcinoma, kidney renal clear cell carcinoma, pancreatic cancer (e.g., pancreatic ductal adenocarcinoma), sarcoma, or stomach adenocarcinoma.

In some embodiments, the cell is situated in a subject.

In certain embodiments, the cell is ex vivo.

In some embodiments, the cell is a mammalian cell, e.g., a human cell.

In certain embodiments, the cell is a somatic cell.

In some embodiments, the cell is a primary cell.

In some embodiments, the step of contacting is performed ex vivo.

In some embodiments, the method further comprises, prior to the step of contacting, a step of removing the cell (e.g., mammalian cell) from a subject.

In some embodiments, the method further comprises, after the step of contacting, a step of administering the cells (e.g., mammalian cells) to a subject.

In certain embodiments, the step of contacting comprises administering a composition comprising the expression repressor to a subject.

In some embodiments, the expression repressor is administered as a monotherapy.

In certain embodiments, the expression repressor is administered in combination with a second therapeutic agent.

In one aspect, the disclosure provides a reaction mixture comprising a cell (e.g., a human cell, e.g., a primary human cell) and an expression repressor, or system of any of the previous aspects or embodiments.

In one aspect, the disclosure provides a method of treating a subject having an inflammatory disorder, comprising:
administering to the subject an expression repressor, system, nucleic acid, nucleic acid system, or reaction mixture of any of the previous aspects or embodiments, in an amount sufficient to treat the disorder (e.g., inflammatory disorder),
thereby treating the disorder (e.g., inflammatory disorder).

In some embodiments, the inflammatory disorder is rheumatoid arthritis, psoriasis, or inflammatory bowel disease.

In some embodiments, the inflammatory disorder is rheumatoid arthritis, gout, neutrophilic asthma, neutrophilic dermatosis, acute respiratory disease syndrome (ARDS), alcohol hepatitis, chronic obstructive pulmonary disease (COPD), or COVID-19.

In certain embodiments, the inflammatory disorder is an autoimmune disorder, e.g., rheumatoid arthritis.

In some embodiments, the inflammatory disease is associated with a pathogenic infection, e.g., viral infection, e.g., SARS-CoV2 infection.

In certain embodiments, the inflammatory disease is associated with a superinfection, e.g., infection caused by two or more pathogenic agents, e.g., by a virus and a bacterium, (e.g., by SARS-CoV2 and Streptococcus pneumoni), e.g., by a virus and a fungus. (e.g., by SARS-CoV2 and mucormycosis).

In one aspect, the disclosure provides a method of treating a subject having cancer, comprising:
administering to the subject an expression repressor, system, nucleic acid, nucleic acid system, or reaction mixture of any of claims 1-102 in an amount sufficient to treat the cancer,
thereby treating the cancer.

In certain embodiments, the cancer is lung cancer (e.g., non-small cell lung cancer), breast cancer, hepatocellular carcinoma (HCC), prostate cancer, colon cancer, skin cancer, cervical cancer, ovarian cancer, uterine endometrioid carcinoma, endometrial cancer, mature B-cell lymphoma, bladder cancer, esophagogastric cancer, esophageal adenocarcinoma, bone cancer, melanoma, hepatobiliary cancer, thyroid cancer, mature B-cell neoplasms, glioma, head-neck squamous cell carcinoma, kidney renal clear cell carcinoma, pancreatic cancer (e.g., pancreatic ductal adenocarcinoma), sarcoma, or stomach adenocarcinoma.

In some embodiments, the subject has an E1 cis-acting regulatory element sequence comprising the sequence of SEQ ID NO: 162, or a sequence with no more than 8, 7, 6, 5, 4, 3, 2, or 1 alterations relative thereto.

In certain embodiments, the subject has an E2 cis-acting regulatory element sequence comprising the sequence of SEQ ID NO: 163, or a sequence with no more than 8, 7, 6, 5, 4, 3, 2, or 1 alterations relative thereto.

In some aspects, the disclosure is directed to a nucleic acid encoding the first expression repressor, second expression repressor, both, or a component thereof (e.g., a gRNA, a mRNA). In some embodiments, the nucleic acid encoding the expression repressor system is a poly-cistronic sequence. In some embodiments, the poly-cistronic sequence is a bi-cistronic sequence.

In some aspects, the present disclosure provides an expression repressor, the expression repressor comprising a targeting moiety that targets an enhancer operably linked to a plurality of genes. In some aspects, the present disclosure provides a method of reducing expression of a plurality of genes, comprising contacting a cell comprising the plurality of genes with an expression repressor, the expression repressor comprising a targeting moiety that targets an enhancer operably linked to the plurality of genes. In some embodiments, the plurality of genes comprise CXCL genes. In some embodiments, the expression repressor targets the E1 cRE of the CXCL locus.

In one aspect, the expression repressor or system comprising an expression repressor may be used in combination with a site-specific disrupting agent described herein. For instance, an expression repressor that targets a cis-acting regulatory element of the CXCL locus may be used in combination with a site-specific disrupting agent that targets an anchor sequence of the CXCL locus. In some embodiments, the site-specific disrupting agent is a site-specific disrupting agent of any one of embodiments B1-B232. In some embodiments, the site-specific disrupting agent is a site-specific disrupting agent described herein. In some embodiments, the site-specific disrupting agent is one described in International Application PCT/US2021/052720, which is incorporated herein by reference in its entirety.

In one aspect, a site-specific disrupting agent comprises a targeting moiety that binds specifically to a first anchor sequence or proximal to the first anchor sequence in an ASMC. In some embodiments, binding of the site-specific disrupting agent occurs in an amount sufficient to modulate, e.g., decrease, expression of the plurality of target genes, e.g., the first gene and second gene. In some embodiments, the site-specific disrupting agent further comprises an effector moiety. Generally, modulation of expression of a target plurality of genes by a site-specific disrupting agent involves the binding of the site-specific disrupting agent to or proximal to the first anchor sequence. In some embodiments, binding of the site-specific disrupting agent to the first anchor sequence may disrupt binding of a nucleating polypeptide, e.g., CTCF, to the first anchor sequence, e.g., thereby disrupting formation and/or maintenance of the ASMC, e.g., and thereby modulating, e.g., decreasing, expression of the plurality of genes. In some embodiments, binding of the site-specific disrupting agent to or proximal to the first anchor sequence may localize a functionality of an effector moiety to the first anchor sequence and/or ASMC, e.g., thereby disrupting formation and/or maintenance of the ASMC, e.g., and thereby modulating, e.g., decreasing, expression of the plurality of genes. In some embodiments, binding of the site-specific disrupting agent to or proximal to the first anchor sequence may localize a functionality of an effector moiety to the first anchor sequence and/or ASMC, e.g., thereby modulating, e.g., decreasing, expression of the plurality of genes. Without wishing to be bound by theory, in some embodiments it is thought that targeting a plurality of genes that are within the same ASMC may more effectively modulate, e.g., decrease, expression of the plurality of genes and/or more effectively achieve a therapeutic effect relating to the functionality of the plurality of genes. For example, in some embodiments, a targeted plurality of genes may all be pro-inflammatory genes; targeting the plurality of pro-inflammatory genes for modulation, e.g., reduction, in expression as taught herein may more effectively decrease inflammation than targeting individual genes. Targeting a plurality of genes comprised within the same genomic complex, e.g., ASMC, (e.g., by targeting the ASMC or an anchor sequence of the ASMC) may have an additive or synergistic effect (e.g., with regard to expression modulation or stability/duration of modulation) that is greater than the effect of targeting individual genes of the plurality.

In some embodiments, a method described herein comprises decreasing expression of a first gene and a second gene in a cell. In some embodiments, the method comprises: contacting the cell with a site-specific disrupting agent comprising a targeting moiety that binds specifically to a first anchor sequence or a site proximal to a first anchor sequence, in an amount sufficient to decrease expression of the first and second genes, the first and second genes being within an anchor sequence-mediated conjunction that comprises the first anchor sequence and a second anchor sequence. In some embodiments, the first gene and the second gene are proinflammatory genes. In some embodiments, the first gene and the second gene are CXCL genes.

In some embodiments, a system described herein comprises, or a method described herein involves the use of, a DNA-binding, e.g., a targeting moiety that binds specifically to or proximal to a first anchor sequence within a cell. In some embodiments, the first anchor sequence is part of an anchor sequence-mediated conjunction that further comprises a second anchor sequence, a first gene, and a second gene. In some embodiments, the first gene and the second gene are CXCL genes.

In some embodiments, a system described herein comprises, or a method described herein involves the use of, a site-specific disrupting agent, comprising: a targeting moiety that binds specifically to or proximal to a first anchor sequence within a cell, wherein the first anchor sequence is part of an anchor sequence-mediated conjunction that further comprises a second anchor sequence, a first gene, and a second gene, wherein the first gene and the second gene are CXCL genes.

In some embodiments, a method described herein comprises decreasing expression of a first gene and a second gene in a cell, the method comprising: contacting the cell with a site-specific disrupting agent that comprises a targeting moiety that binds specifically to a first anchor sequence or a site proximal to a first anchor sequence, in an amount sufficient to decrease expression of the first and second genes, the first and second genes being within an anchor sequence-mediated conjunction that comprises the first anchor sequence and a second anchor sequence, wherein the first gene and the second gene are CXCL genes; thereby decreasing expression of the first and second genes.

In another aspect, the disclosure is directed to a reaction mixture comprising a cell (e.g., a human cell, e.g., a primary human cell) a system as described herein (e.g., a system comprising an expression repressor described herein and optionally further comprising a site-specific disrupting agent described herein).

In another aspect, the disclosure is directed to a method of treating a subject having an inflammatory disorder, comprising administering to the subject a system as described herein (e.g., a system comprising an expression repressor described herein and optionally further comprising a site-specific disrupting agent described herein) in an amount sufficient to treat the inflammatory disorder.

In another aspect, the disclosure is directed to a method of treating inflammation, e.g., local inflammation, in a subject having an infection, e.g., viral infection, e.g., COVID-19, comprising, administering to the subject a system as described herein (e.g., a system comprising an expression repressor described herein and optionally further comprising a site-specific disrupting agent described herein) in an amount sufficient to treat the inflammation.

In another aspect, the disclosure is directed to a human cell having decreased expression of a first gene and a second gene, wherein the first gene and the second gene are proinflanmmatory genes, wherein the cell comprises a disrupted (e.g., fully disrupted) anchor sequence-mediated conjunction that comprises the first and second genes. In some embodiments, the human cell was previously contacted with a system described herein (e.g., a system comprising an expression repressor described herein and optionally further comprising a site-specific disrupting agent described herein). In some embodiments, the human cell no longer comprises a system described herein.

In some embodiments, a human cell described herein comprises a mutation at genomic coordinates chr4:74595464-74595486, chr4:74595457-74595479, chr4:74595460-74595482, chr4:74595472-74595494, chr4:75000088-75000110, chr4:75000091-75000113, chr4:75000085-75000107, chr4:75000157-75000179, chr4:75000156-75000178, chr4:74595215-74595237, chr4:74595370-74595392, chr4:74595560-74595582, chr4:74595642-74595664, chr4:74595787-74595809, chr4:74528428-74528450, chr4:74528567-74528589, chr4:74528609-74528631, chr4:74789132-74789154, chr4:74789250-74789272, chr4:74789312-74789334, chr4:74964853-74964875, chr4:74964906-74964928, chr4:74965538-74965560, chr4:74965737-74965759, chr4:75000031-75000053, chr4:75000115-75000137, chr4:75000231-75000253, chr4:74975146-74975168, chr4:74975369-74975391, chr4:74976318-74976340, chr4:74570348-74570370, chr4:74570503-74570525, or chr4:74570526-74570548, or within 5, 10, 15, 20, 30, 40, or 50 nucleotides of said region.

### Numbered Embodiments B

B1. A method of decreasing expression of a first gene and a second gene in a cell, comprising:
contacting the cell with a site-specific disrupting agent that comprises a targeting moiety that binds specifically to a first anchor sequence or a site proximal to a first anchor sequence, in an amount sufficient to decrease expression of the first and second genes,
the first and second genes being within an anchor sequence-mediated conjunction that comprises the first anchor sequence and a second anchor sequence,
wherein optionally the first gene and the second gene are proinflammatory genes;
thereby decreasing expression of the first and second genes.
B2. A site-specific disrupting agent, comprising:
a DNA-binding, e.g., a targeting moiety that binds specifically to or proximal to a first anchor sequence within a cell,
wherein the first anchor sequence is part of an anchor sequence-mediated conjunction that further comprises a second anchor sequence, a first gene, and a second gene,
wherein optionally the first gene and the second gene are proinflammatory genes.
B3. The site-specific disrupting agent of embodiment B2, wherein the first or second anchor sequence is located between IL-8 and RASSF6; between the IL-8 enhancer and RASSF6; between CXCL1 and CXCL4; between CXCL2 and EPGN; or between the E2 enhancer and EPGN.
B4. The site-specific disrupting agent of embodiment B2 or B3, wherein the site-specific disrupting agent further comprises an effector moiety.
B5. The site-specific disrupting agent of any of embodiments B2-B4 wherein the targeting moiety comprises a TAL effector molecule, a CRISPR/Cas molecule (e.g., a catalytically inactive CRISPR/Cas protein), a zinc finger domain, a tetR domain, a meganuclease, or an oligonucleotide.
B6. The site-specific disrupting agent of any of embodiments B2-B5, wherein the effector moiety comprises an effector described herein, e.g., MQ1, DNMT1, DNMT3A1, DNMT3A2, DNMT3B1, DNMT3B2, DNMT3B3, DNMT3B4, DNMT3B5, DNMT3B6, DNMT3L, EZH2, HDAC8, KRAB, MeCP2, HP1, RBBP4, REST, FOG1, SUZ12, SETDB1, SETDB2, EHMT2 (i.e., G9A), EHMT1 (i.e., GLP), SUV39H1, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, EZH1, SUV39H2, SETD8, SUV420H1, SUV420H2 or DNMT3, or a functional variant or fragment of any thereof.
B7. The site-specific disrupting agent of any of embodiments B2-B6, wherein the effector moiety is linked to the targeting moiety via a linker.
B8. The site-specific disrupting agent of any of embodiments B2-B7, wherein the effector moiety is C-terminal of the targeting moiety.
B9. The site-specific disrupting agent of any of embodiments B2-B7, wherein the effector moiety is N-terminal of the targeting moiety.
B10. The site-specific disrupting agent of any of embodiments B2-B9, wherein the effector moiety is encoded by a nucleotide sequence chosen from any of SEQ ID NOs: 10, 14, 16, 18, 66, 68, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
B11. The site-specific disrupting agent of any of embodiments B2-B10, wherein the effector moiety comprises an amino acid sequence according to any of SEQ ID NOs: 11, 12, 13, 15, 17, 19, 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
B12. The site-specific disrupting agent of any of embodiments B2-B11, wherein the effector moiety is MQ1 or a functional variant or fragment thereof, e.g., wherein the effector moiety comprises an amino acid sequence of SEQ ID NO: 11 or 12 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the effector moiety is C-terminal of the targeting moiety.
B13. The site-specific disrupting agent of any of embodiments B2-B11, wherein the effector moiety is KRAB, or a functional variant or fragment thereof, e.g., wherein the effector moiety comprises an amino acid sequence of SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the effector moiety is C-terminal of the targeting moiety.
B14. The site-specific disrupting agent of any of embodiments B2-B11, wherein the effector moiety is DNMT3a/3L, or a functional variant or fragment thereof, e.g., wherein the effector moiety comprises an amino acid sequence of SEQ ID NO: 15 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the effector moiety is C-terminal of the targeting moiety.
B15. The site-specific disrupting agent of any of embodiments B2-B11, wherein the effector moiety is EZH2, or a functional variant or fragment thereof, e.g., wherein the effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
B16. The site-specific disrupting agent of any of embodiments B2-B11, wherein the effector moiety is HDAC8, or a functional variant or fragment thereof, e.g., wherein the effector moiety comprises an amino acid sequence of SEQ ID NO: 19 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the effector moiety is C-terminal of the targeting moiety.
B17. The site-specific disrupting agent of any of embodiments B2-B11, wherein the effector moiety is G9A, or a functional variant or fragment thereof, e.g., wherein the effector moiety comprises an amino acid sequence of SEQ ID NO: 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the effector moiety is N-terminal of the targeting moiety.
B18. The site-specific disrupting agent of any of embodiments B2-B17, which further comprises a second effector moiety.
B19. The site-specific disrupting agent of embodiment B18, wherein the targeting moiety is situated between the first effector moiety and the second effector moiety.
B20. The site-specific disrupting agent of any of embodiments B2-B19, wherein the effector moiety comprises a polymer e.g., an oligonucleotide; e.g., a gRNA.
B21. The site-specific disrupting agent of embodiment B20, wherein the oligonucleotide has a sequence that comprises a complement of the anchor sequence or to a sequence proximal to the anchor sequence.
B22. The site-specific disrupting agent of any of embodiments B2-B21, wherein the targeting moiety further comprises a gRNA, e.g., a gRNA that binds a genomic locus comprising at least 14, 15, 16, 17, 18, 19, or 20 nucleotides of the sequence of any of SEQ ID NOs: 20-62, e.g., wherein the gRNA comprises a sequence that comprises at least 14, 15, 16, 17, 18, 19, or 20 nucleotides of the sequence of any of SEQ ID NOs: 20-62.
B23. The site-specific disrupting agent of any of embodiments B2-B22, wherein the targeting domain comprises a CRISPR/Cas molecule, e.g., a catalytically inactive CRISPR/Cas protein, and a gRNA, e.g., a gRNA that binds a genomic locus comprising at least 14, 15, 16, 17, 18, 19, or 20 nucleotides of the sequence of any of SEQ ID NOs: 20-62, e.g., wherein the gRNA comprises a sequence that comprises at least 14, 15, 16, 17, 18, 19, or 20 nucleotides of the sequence of any of SEQ ID NOs: 20-62 and the effector moiety comprises an effector chosen from DNMT3a/3l, MQ1, KRAB, G9A, HDAC8, or EZH2.
B24. The site-specific disrupting agent of embodiment B23, wherein the targeting domain comprises a CRISPR/Cas molecule, e.g., a catalytically inactive CRISPR/Cas protein, and a gRNA, e.g., a gRNA that binds a genomic locus comprising at least 14, 15, 16, 17, 18, 19, or 20 nucleotides of the sequence of any of SEQ ID NOs: 20-62, e.g., wherein the gRNA comprises a sequence that comprises at least 14, 15, 16, 17, 18, 19, or 20 nucleotides of the sequence of any of SEQ ID NOs: 20-62, the first effector moiety comprises an effector chosen from DNMT3a/3l, MQ1, KRAB, G9A, HDAC8, or EZH2, and the second effector moiety comprises an effector chosen from DNMT3a/3l, MQ1 KRAB, G9A, HDAC8, or EZH2.
B25. The site-specific disrupting agent of any of embodiments B2-B24, wherein the targeting domain binds a genomic locus comprising at least 14, 15, 16, 17, 18, 19, or 20 nucleotides of the sequence of any of SEQ ID NOs: 20-62.
B26. The site-specific disrupting agent of any of embodiments B2-B25, wherein the targeting domain binds a genomic locus that is within 50 nucleotides (e.g., upstream or downstream) of the sequence of any of SEQ ID NOs: 20-62.
B27. The site-specific disrupting agent of any of embodiments B2-B26, wherein the targeting domain binds a genomic locus that is within 100 nucleotides (e.g., upstream or downstream) of the sequence of any of SEQ ID NOs: 20-62.
B28. The site-specific disrupting agent of any of embodiments B2-B27, wherein the targeting domain binds a genomic locus that is within 200 (e.g., upstream or downstream) nucleotides of the sequence of any of SEQ ID NOs: 20-62.
B29. The site-specific disrupting agent of any of embodiments B2-B28, wherein the targeting domain binds a genomic locus that is within 300 nucleotides (e.g., upstream or downstream) of the sequence of any of SEQ ID NOs: 20-62.
B30. The site-specific disrupting agent of any of embodiments B2-B29, which: (i) comprises one or more nuclear localization signal sequences (NLS), or (ii) does not comprise an NLS, optionally wherein the NLS comprises an amino acid sequence of SEQ ID NO: 63 and/or 64.
B31. The site-specific disrupting agent of any of embodiments B18-B30, wherein the first and/or second effector moiety comprises a DNA methyltransferase, a histone methyltransferase, a histone deacetylase, a histone demethylase, or a recruiter of a histone modifying complex.
B32. The site-specific disrupting agent of embodiment B2-B31, wherein the ASMC comprises two loops.
B33. The site-specific disrupting agent of any of embodiments B2-B32 or the method of embodiment B1, wherein the first gene is situated in a first loop of the ASMC, and the second gene is situated in a second loop of the ASMC.
B34. The site-specific disrupting agent or method of embodiment B33, wherein the first anchor sequence is situated between the first and second loops.
B35. A nucleic acid encoding a site-specific disrupting agent of any of embodiments B2-B34.
B36. The method of embodiment B1 or site-specific disrupting agent of any of embodiments B2-B36, wherein the anchor sequence-mediated conjunction further comprises a third gene, and optionally wherein the method results in decreased expression of the third gene.
B37. The method or site-specific disrupting agent of embodiment B36, wherein the anchor sequence-mediated conjunction further comprises a fourth gene, and optionally wherein the method results in decreased expression of the fourth gene.
B38. The method or site-specific disrupting agent of embodiment B37, wherein the anchor sequence-mediated conjunction further comprises a fifth gene, and optionally wherein the method results in decreased expression of the fifth gene.
B39. The method or site-specific disrupting agent of embodiment B38, wherein the anchor sequence-mediated conjunction further comprises a sixth gene, and optionally wherein the method results in decreased expression of the sixth gene.
B40. The method or site-specific disrupting agent of embodiment B39, wherein the anchor sequence-mediated conjunction further comprises a seventh gene, and optionally wherein the method results in decreased expression of the seventh gene.
B41. The method or site-specific disrupting agent of embodiment B40, wherein the anchor sequence-mediated conjunction further comprises an eighth gene, and optionally wherein the method results in decreased expression of the eighth gene.
B42. A human cell having decreased expression of a first gene and a second gene, wherein the first gene and the second gene are proinflammatory genes, wherein the cell comprises a disrupted (e.g., fully disrupted) anchor sequence-mediated conjunction that comprises the first and second genes.
B43. The human cell of embodiment B42, which has reduced CTCF binding to an anchor sequence that is comprised by the anchor sequence-mediated conjunction, e.g., reduced by at least 20, 30, 40, 50, 60, 70, 80, 90, or 100%.
B44. The human cell of either of embodiment B42 or B43, wherein the human cell has decreased expression of a third proinflammatory gene.
B45. The human cell of embodiment B44, wherein the human cell has decreased expression of a fourth proinflammatory gene.
B46. The human cell of embodiment B45, wherein the human cell has decreased expression of a fifth proinflammatory gene.
B47. The human cell of embodiment B46, wherein the human cell has decreased expression of a sixth proinflammatory gene.
B48. The human cell of embodiment B47, wherein the human cell has decreased expression of a seventh proinflammatory gene.
B49. The human cell of embodiment B48, wherein the human cell has decreased expression of an eighth proinflammatory gene.
B50. The human cell of any of embodiments B42-B49, wherein the human cell comprises a mutation at chr4:74595464-74595486, chr4:74595457-74595479, chr4:74595460-74595482, chr4:74595472-74595494, chr4:75000088-75000110, chr4:75000091-75000113, chr4:75000085-75000107, chr4:75000157-75000179, chr4:75000156-75000178, chr4:74595215-74595237, chr4:74595370-74595392, chr4:74595560-74595582, chr4:74595642-74595664, chr4:74595787-74595809, chr4:74528428-74528450, chr4:74528567-74528589, chr4:74528609-74528631, chr4:74789132-74789154, chr4:74789250-74789272, chr4:74789312-74789334, chr4:74964853-74964875, chr4:74964906-74964928, chr4:74965538-74965560, chr4:74965737-74965759, chr4:75000031-75000053, chr4:75000115-75000137, chr4:75000231-75000253, chr4:74975146-74975168, chr4:74975369-74975391, chr4:74976318-74976340, chr4:74570348-74570370, chr4:74570503-74570525, or chr4:74570526-74570548, or within 5, 10, 15, 20, 30, 40, or 50 nucleotides of said region.
B51. A human cell comprising a mutation at chr4:74595464-74595486, chr4:74595457-74595479, chr4:74595460-74595482, chr4:74595472-74595494, chr4:75000088-75000110, chr4:75000091-75000113, chr4:75000085-75000107, chr4:75000157-75000179, chr4:75000156-75000178, chr4:74595215-74595237, chr4:74595370-74595392, chr4:74595560-74595582, chr4:74595642-74595664, chr4:74595787-74595809, chr4:74528428-74528450, chr4:74528567-74528589, chr4:74528609-74528631, chr4:74789132-74789154, chr4:74789250-74789272, chr4:74789312-74789334, chr4:74964853-74964875, chr4:74964906-74964928, chr4:74965538-74965560, chr4:74965737-74965759, chr4:75000031-75000053, chr4:75000115-75000137, chr4:75000231-75000253, chr4:74975146-74975168, chr4:74975369-74975391, chr4:74976318-74976340, chr4:74570348-74570370, chr4:74570503-74570525, or chr4:74570526-74570548, or within 5, 10, 15, 20, 30, 40, or 50 nucleotides of said region.
B52. The human cell of either of embodiments B27 or B28, wherein the mutation comprises a deletion, substitution, or insertion (e.g., of 1-10, 1-9, 1-8, 1-7, 1-6, 1-5, 1-4, 1-3, or 1-2 nucleotides).
B53. The human cell of any of embodiments B50-B52, which has reduced CTCF binding to the mutation, e.g., reduced by at least 20, 30, 40, 50, 60, 70, 80, 90, or 100% compared to a human cell with an undisrupted ASMC.
B54. The human cell of any of embodiments B42-B53, wherein expression of the first and second genes is reduced by at least 10%, 20%, 30%, 40%, 50%, 60%, 70%, 80%, or 90% compared to a human cell with an undisrupted ASMC.
B55. A system comprising:
a first site-specific disrupting agent comprising a first targeting moiety and optionally a first effector moiety, wherein the first site-specific disrupting agent binds specifically to a first anchor sequence of an anchor sequence mediated conjunction (ASMC), wherein the ASMC comprises a first gene and a second gene, and
a second site-specific disrupting agent comprising a second targeting moiety and optionally a second effector moiety, wherein the second site-specific disrupting agent binds to a second anchor sequence of the ASMC.
B56. The system of embodiment B55, wherein the first anchor sequence is between IL-8 and RASSF6; between the IL-8 enhancer and RASSF6; between CXCL1 and CXCL4; between CXCL2 and EPGN; or between the E2 enhancer and EPGN.
B57. The system of embodiment B55 or B56, wherein the second anchor sequence is between IL-8 and RASSF6; between the IL-8 enhancer and RASSF6; between CXCL1 and CXCL4; between CXCL2 and EPGN; or between the E2 enhancer and EPGN.
B58. The system of any of embodiments B55-B57, wherein the first anchor sequence is between the IL-8 enhancer and RASSF6 and the second anchor sequence is between CXCL1 and CXCL4.
B59. The system of any of embodiments B55-B58, wherein the first anchor sequence is between the IL-8 enhancer and RASSF6 and the second anchor sequence is between the E2 enhancer and EPGN.
B60. The system of any of embodiments B55-B59, wherein the first anchor sequence is between CXCL1 and CXCL4 and the second anchor sequence is between the E2 enhancer and EPGN.
B61. The system of any of embodiments B55-B60, wherein the first site-specific disrupting agent is a site-specific disrupting agent described herein, e.g., a site-specific disrupting agent of any of embodiments B2-B9.
B62. The system of any of embodiments B55-B61, wherein the second site-specific disrupting agent is a site-specific disrupting agent described herein, e.g., a site-specific disrupting agent of any of embodiments B2-B9.
B63. The system of any of embodiments B55-B62, wherein the first targeting moiety and the second targeting moiety each independent comprises a TAL effector molecule, a CRISPR/Cas molecule, a zinc finger domain, a tetR domain, a meganuclease, or an oligonucleotide.
B64. The system of any of embodiments B55-B63, wherein the first effector and the second effector each independently comprises an effector described herein, e.g., MQ1, EZH2, HDAC8, KRAB, G9A, or DNMT3a/3l, or a functional variant or fragment of any thereof.
B65. The system of any of embodiments B55-B62, wherein the first effector and the second effector each independently comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof.
B66. The system of any of embodiments B55-B65, wherein the first effector and the second effector each independently comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.
B67. The system of any of embodiments B55-B43, wherein the first effector and the second effector each independently comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3l, or a functional variant or fragment of any thereof.
B68. The system of any of embodiments B55-B67, wherein the first effector and the second effector each independently comprises a protein chosen from KRAB, MeCP2, HP1, RBBP4, REST, FOG1, SUZ12, or a functional variant or fragment of any thereof.
B69. The system of any of embodiments B55-B68, wherein the first effector and the second effector each independently comprises a polymer e.g., an oligonucleotide.
B70. The system of any of embodiments B55-B69, wherein the first oligonucleotide and the second oligonucleotide are identical.
B71. The system of any of embodiments B55-B70, wherein the first oligonucleotide and the second oligonucleotide are different.
B72. The system of any of embodiments B55-B71, wherein the first oligonucleotide has a sequence that comprises a complement of the first anchor sequence or to a sequence proximal to the first anchor sequence and the second oligonucleotide has a sequence that comprises a complement of the second anchor sequence or to a sequence proximal to the second anchor sequence.
B73. The system of any of embodiments B55-B72, wherein the anchor sequence-mediated conjunction further comprises a third gene.
B74. The system of any of embodiments B55-B73, wherein the anchor sequence-mediated conjunction further comprises a fourth gene.
B75. The system of any of embodiments B55-B74, wherein the anchor sequence-mediated conjunction further comprises a fifth gene.
B76. The system of any of embodiments B55-B75, wherein the anchor sequence-mediated conjunction further comprises a sixth gene.
B77. The system of any of embodiments B55-B76, wherein the anchor sequence-mediated conjunction further comprises a seventh gene.
B78. The system of any of embodiments B55-B77, wherein the anchor sequence-mediated conjunction further comprises an eighth gene.
B79. The system of any of embodiments B55-B78, wherein the ASMC comprises two loops.
B80. A nucleic acid composition encoding the system of any of embodiments B55-B79.
B81. The nucleic acid of embodiment B80, wherein a single nucleic acid encodes both of the first site-specific disrupting agent and the second site-specific disrupting agent.
B82. The nucleic acid of embodiment B81, wherein a first nucleic acid encodes the first site-specific disrupting agent and a second nucleic acid encodes the second site-specific disrupting agent.
B83. A method of decreasing expression of a first gene and a second gene in a cell, comprising contacting the cell with a system according to any of embodiments B55-B79 of a nucleic acid composition according to any of embodiments B80-B82.
B84. The method of embodiment B83, wherein the cell is simultaneously contacted with the first site-specific disrupting agent and the second site-specific disrupting agent.
B85. The method of embodiment B83, wherein the cell is sequentially contacted with the first site-specific disrupting agent and the second site-specific disrupting agent.
B86. The method, human cell, site-specific disrupting agent, or system of any of embodiments B1-B85, wherein the first gene is CXCL1 and the second gene is CXCL2.
B87. The method, human cell, site-specific disrupting agent, or system of any of embodiments B1-B85, wherein the first gene is CXCL1 and the second gene is CXCL3.
B88. The method, human cell, site-specific disrupting agent, or system of any of embodiments B1-B85, wherein the first gene is CXCL1 and the second gene is IL-8.
B89. The method, human cell, site-specific disrupting agent, or system of any of embodiments B1-B85, wherein the first gene is CXCL1 and the second gene is CXCL4.
B90. The method, human cell, site-specific disrupting agent, or system of any of embodiments B1-B85, wherein the first gene is CXCL1 and the second gene is CXCL5.
B91. The method, human cell, site-specific disrupting agent, or system of any of embodiments B1-B85, wherein the first gene is CXCL1 and the second gene is CXCL6.
B92. The method, human cell, site-specific disrupting agent, or system of any of embodiments B1-B85, wherein the first gene is CXCL1 and the second gene is CXCL7.
B93. The method, human cell, site-specific disrupting agent, or system of any of embodiments B1-B85, wherein the first gene is CXCL2 and the second gene is CXCL3.
B94. The method, human cell, site-specific disrupting agent, or system of any of embodiments B1-B85, wherein the first gene is CXCL2 and the second gene is IL-8.
B95. The method, human cell, site-specific disrupting agent, or system of any of embodiments B1-B85, wherein the first gene is CXCL2 and the second gene is CXCL4.
B96. The method, human cell, site-specific disrupting agent, or system of any of embodiments B1-B85, wherein the first gene is CXCL2 and the second gene is CXCL4.
B97. The method, human cell, site-specific disrupting agent, or system of any of embodiments B1-B85, wherein the first gene is CXCL2 and the second gene is CXCL5.
B98. The method, human cell, site-specific disrupting agent, or system of any of embodiments B1-B85, wherein the first gene is CXCL2 and the second gene is CXCL6.
B99. The method, human cell, site-specific disrupting agent, or system of any of embodiments B1-B85, wherein the first gene is CXCL2 and the second gene is CXCL7.
B100. The method, human cell, site-specific disrupting agent, or system of any of embodiments B1-B85, wherein the first gene is CXCL3 and the second gene is IL-8.
B101. The method, human cell, site-specific disrupting agent, or system of any of embodiments B1-B85, wherein the first gene is CXCL3 and the second gene is CXCL4.
B102. The method, human cell, site-specific disrupting agent, or system of any of embodiments B1-B85, wherein the first gene is CXCL3 and the second gene is CXCL5.
B103. The method, human cell, site-specific disrupting agent, or system of any of embodiments B1-B85, wherein the first gene is CXCL3 and the second gene is CXCL6.
B104. The method, human cell, site-specific disrupting agent, or system of any of embodiments B1-B85, wherein the first gene is CXCL3 and the second gene is CXCL7.
B105. The method, human cell, site-specific disrupting agent, or system of any of embodiments B1-B85, wherein the first gene is CXCL4 and the second gene is CXCL5.
B106. The method, human cell, site-specific disrupting agent, or system of any of embodiments B1-B85, wherein the first gene is CXCL4 and the second gene is CXCL6.
B107. The method, human cell, site-specific disrupting agent, or system of any of embodiments B1-B85, wherein the first gene is CXCL4 and the second gene is CXCL7.
B108. The method, human cell, site-specific disrupting agent, or system of any of embodiments B1-B85, wherein the first gene is CXCL4 and the second gene is IL-8.
B109. The method, human cell, site-specific disrupting agent, or system of any of embodiments B1-B85, wherein the first gene is CXCL5 and the second gene is CXCL6.
B110. The method, human cell, site-specific disrupting agent, or system of any of embodiments B1-B85, wherein the first gene is CXCL5 and the second gene is CXCL7.
B111. The method, human cell, site-specific disrupting agent, or system of any of embodiments B1-B85, wherein the first gene is CXCL5 and the second gene is IL-8.
B112. The method, human cell, site-specific disrupting agent, or system of any of embodiments B1-B85, wherein the first gene is CXCL6 and the second gene is CXCL7.
B113. The method, human cell, site-specific disrupting agent, or system of any of embodiments B1-B85, wherein the first gene is CXCL6 and the second gene is IL-8.
B114. The method, human cell, site-specific disrupting agent, or system of any of embodiments B1-B85, wherein the first gene is CXCL7 and the second gene is IL-8.
B115. The method, human cell, site-specific disrupting agent, or system of any of embodiments B36-B85, wherein the first gene is CXCL1, the second gene is CXCL2, and the third gene is CXCL3.
B116. The method, human cell, site-specific disrupting agent, or system of any of embodiments B36-B85, wherein the first, second, and third genes are chosen from CXCL1, CXCL2, CXCL3, CXCL4, CXCL5, CXCL6, CXCL7, or IL-8.
B117. The method, human cell, site-specific disrupting agent, or system of any of embodiments B36-B85, wherein the first, second, third, and fourth genes are chosen from CXCL1, CXCL2, CXCL3, CXCL4, CXCL5, CXCL6, CXCL7, or IL-8.
B118. The method, human cell, site-specific disrupting agent, or system of any of embodiments B36-B85, wherein the first, second, third, fourth, and fifth genes are chosen from CXCL1, CXCL2, CXCL3, CXCL4, CXCL5, CXCL6, CXCL7, or IL-8.
B119. The method, human cell, site-specific disrupting agent, or system of any of embodiments B36-B85, wherein the first, second, third, fourth, fifth, and sixth genes are chosen from CXCL1, CXCL2, CXCL3, CXCL4, CXCL5, CXCL6, CXCL7, or IL-8.
B120. The method, human cell, site-specific disrupting agent, or system of any of embodiments B36-B85, wherein the first, second, third, fourth, fifth, sixth, and seventh genes are chosen from CXCL1, CXCL2, CXCL3, CXCL4, CXCL5, CXCL6, CXCL7, or IL-8.
B121. The method, human cell, site-specific disrupting agent, or system of any of embodiments B36-B85, wherein the first, second, third, fourth, fifth, sixth, seventh, and eighth genes are CXCL1, CXCL2, CXCL3, CXCL4, CXCL5, CXCL6, CXCL7, or IL-8.
B122. The method, human cell, site-specific disrupting agent, or system of any of the preceding embodiments, wherein the first gene is a cytokine.
B123. The method, human cell, site-specific disrupting agent, or system of any of the preceding embodiments, wherein the second gene is a cytokine.
B124. The method, human cell, site-specific disrupting agent, or system of any of the preceding embodiments, wherein the third gene is a cytokine.
B125. The method, human cell, site-specific disrupting agent, or system of any of the preceding embodiments, wherein the fourth gene is a cytokine.
B126. The method, human cell, site-specific disrupting agent, or system of any of the preceding embodiments, wherein the fifth gene is a cytokine.
B127. The method, human cell, site-specific disrupting agent, or system of any of the preceding embodiments, wherein the sixth gene is a cytokine.
B128. The method, human cell, site-specific disrupting agent, or system of any of the preceding embodiments, wherein the seventh gene is a cytokine.
B129. The method, human cell, site-specific disrupting agent, or system of any of the preceding embodiments, wherein the eighth gene is a cytokine.
B130. The method, human cell, site-specific disrupting agent, or system of any of the preceding embodiments, wherein the anchor sequence-mediated conjunction comprises 3, 4, or 5 proinflammatory genes.
B131. The method, site-specific disrupting agent, or system of any of the preceding embodiments, wherein the site-specific disrupting agent comprises a nucleic acid (e.g., DNA or RNA) comprising a nucleotide sequence chosen from SEQ ID NOs: 20-62, or a sequence having at least 90%, 95%, 98%, or 99% identity thereto, or differing at no more than 1, 2, 3, 4, or 5 positions relative thereto.
B132. The method, site-specific disrupting agent of any of the preceding embodiments, wherein the site-specific disrupting agent comprises a nucleic acid (e.g., DNA or RNA) comprising a nucleotide sequence chosen from SEQ ID NOs: 21, 22, 24, 40, or a sequence having at least 90%, 95%, 98%, or 99% identity thereto, or differing at no more than 1, 2, 3, 4, or 5 positions relative thereto.
B133. The method or site-specific disrupting agent of any of the preceding embodiments, wherein the site-specific disrupting agent binds to a sequence at least partially overlapping with the region having genomic coordinates chosen from Table 4 5, 6, 7, or a sequence that is within 5, 10, 15, 20, 30, 40, 50, 100, 200, 300, 400, 500, 600, 700, 800, 900, or 1000 nucleotides of said region.
B134. The method of any of the preceding embodiments, which results in a decrease in a level of a cytokine, e.g., a chemokine, e.g., upon stimulation of the cell with TNF-alpha, e.g., measured as described in Examples 2-11.
B135. The method or human cell of any of the preceding embodiments, wherein a level of a cytokine (e.g., a chemokine) is decreased, e.g., upon stimulation of the cell with TNF-alpha, e.g., measured as described in Examples 2-11.
B136. The method or human cell of any of the preceding embodiments, wherein the transcript level of one or more of (e.g., 2, 3, or all of) CXCL1, CXCL2, CXCL3, and IL8 is decreased, e.g., upon stimulation of the cell with TNF-alpha, e.g., measured as described in Examples 2 or 4-11.
B137. The method or human cell of any of the preceding embodiments, wherein the transcript level of one or more of (e.g., 2, 3, or all of) CXCL1, CXCL2, CXCL3, CXCL4, CXCL5, CXCL6, CXCL7, and IL8 is decreased, e.g., upon stimulation of the cell with TNF-alpha.
B138. The method or human cell of any of embodiments B132-B137, wherein the decrease is a decrease of at least 10%, 20%, 30%, 40%, 50%, 60%, 70%, 80%, or 90% compared to pre-treatment levels or to a human cell with an undisrupted ASMC.
B139. The method or human cell of any of the preceding embodiments, wherein the protein level (e.g., secreted protein level) of one or more of (e.g., 2, 3, or all of) of CXCL1, CXCL2, CXCL3, and IL8 is decreased, e.g., upon stimulation of the cell with TNF-alpha, e.g., measured as described Example 3.
B140. The method or human cell of any of the preceding embodiments, wherein the protein level (e.g., secreted protein level) of one or more of (e.g., 2, 3, or all of) CXCL1, CXCL2, CXCL3, CXCL4, CXCL5, CXCL6, CXCL7, and IL8 is decreased, e.g., upon stimulation of the cell with TNF-alpha.
B141. The method or human cell of embodiment B140, wherein the decrease is a decrease of at least 10%, 20%, 30%, 40%, 50%, 60%, 70%, 80%, or 90% compared to pre-treatment levels or to a human cell with an undisrupted ASMC.
B142. The method of any of the preceding embodiments, which results in decrease in binding of CTCF to the first anchor sequence, e.g., a complete loss of binding or a loss of at least 20, 30, 40, 50, 60, 70, 80, 90, or 100% compared to a human cell with an undisrupted ASMC, e.g., as measured by ChIP and quantitative PCR.
B143. The method of any of the preceding embodiments, which results in disruption of the anchor sequence-mediated conjunction.
B144. The method of any of the preceding embodiments, wherein a population of the cells is contacted with the site-specific disrupting agent, and wherein the first anchor sequence is edited in at least 50%, 60%, 70%, 80%, 90%, or 95% of cells in the population.
B145. The method of any of the preceding embodiments, wherein the effect (e.g., the decrease in cytokine levels) is additive or synergistic compared to the effect of inhibiting the first gene or the second gene individually.
B146. The method of any of the preceding embodiments, wherein expression is decreased for at least 1, 2, 3, 4, 5, 6, 7, 10, or 14 days, or at least 1, 2, 3, 4, or 5 weeks.
B147. The method, human cell, site-specific disrupting agent, or system of any of the preceding embodiments, wherein the cell is a cell of a subject having an inflammatory disease, e.g., an immune mediated inflammatory disease.
B148. The method, human cell, site-specific disrupting agent, or system of any of the preceding embodiments, wherein the inflammatory disease is an autoimmune disorder, e.g., rheumatoid arthritis.
B149. The method, human cell, site-specific disrupting agent, or system of any of the preceding embodiments, wherein the inflammatory disease is associated with a pathogenic infection, e.g., viral infection, e.g., SARS-CoV2 infection.
B150. The method, human cell, site-specific disrupting agent, or system of any of the preceding embodiments, wherein the inflammatory disease is associated with a superinfection, e.g., infection caused by two or more pathogenic agents, e.g., by a virus and a bacterium, (e.g., by SARS-CoV2 and Streptococcus pneumoni), e.g., by a virus and a fungus, (e.g., by SARS-CoV2 and mucormycosis).
B151. The method, human cell, site-specific disrupting agent, or system of any of the preceding embodiments, wherein the cell is a cell of a subject having rheumatoid arthritis, inflammatory, arthritis, gout, asthma, neutrophilic asthma, neutrophilic dermatosis, paw edema, acute respiratory disease syndrome (ARDS), COVID-19, psoriasis, inflammatory bowel disease, infection (e.g., by a pathogen, e.g., a bacteria, a viruses, or a fungus), external injury (e.g., scrapes or foreign objects), effects of radiation or chemical injury, osteoarthritis, osteoarthritic joint pain, joint pain, inflammatory pain, acute pain, chronic pain, cystitis, bronchitis, dermatitis, dermatosis, cardiovascular disease, neurodegenerative disease, liver disease, lung disease, kidney disease, pain, swelling, stiffness, tenderness, redness, warmth, or elevated biomarkers related to disease states (e.g., cytokines, chemokines, growth factors, immune receptors, infection markers, or inflammatory markers).
B152. The method, human cell, site-specific disrupting agent, or system of any of the preceding embodiments, wherein the cell is a cell of a subject having rheumatoid arthritis, psoriasis, or inflammatory bowel disease.
B153. The method, human cell, site-specific disrupting agent, or system of any of the preceding embodiments, wherein the cell is a cell of a subject having rheumatoid arthritis, gout, neutrophilic asthma, neutrophilic dermatosis, acute respiratory disease syndrome (ARDS), or COVID-19.
B154. The method, site-specific disrupting agent, or system of any of the preceding embodiments, wherein the anchor sequence-mediated conjunction comprises an internal enhancing sequence.
B155. The method, site-specific disrupting agent, or system of any of the preceding embodiments, wherein the second gene (and optionally the third, fourth, fifth, sixth, seventh, or eighth genes) is transcribed in the same direction as the first gene.
B156. The method, site-specific disrupting agent, or system of any of the preceding embodiments, wherein the first anchor sequence comprises a binding motif selected from a CTCF binding motif, USF1 binding motif, YY1 binding motif, TAF3 binding motif, or ZNF143 binding motif.
B157. The method, site-specific disrupting agent, or system of any of the preceding embodiments, wherein the first anchor sequence comprises a CTCF binding motif.
B158. The method, site-specific disrupting agent, or system of any of the preceding embodiments, wherein the site-specific disrupting agent binds specifically to or proximal to the first anchor sequence with sufficient affinity that it competes with binding of an endogenous nucleating polypeptide (e.g., CTCF, USF1, YY1, TAF3, or ZNF143) within the cell.
B159. The method, site-specific disrupting agent, or system of any of the preceding embodiments, wherein the site-specific disrupting agent adds, deletes, or substitutes one or more nucleotides within or proximal to the first anchor sequence.
B160. The method or site-specific disrupting agent of any of the preceding embodiments, wherein the site-specific disrupting agent comprises a targeting moiety or effector moiety comprising a first CRISPR/Cas molecule comprising a first CRISPR/Cas protein and first guide RNA.
B161. The method or system of any of the preceding embodiments, wherein the first site-specific disrupting agent comprises a first targeting moiety or first effector moiety comprising a first CRISPR/Cas molecule comprising a first CRISPR/Cas protein and first guide RNA and the second site-specific disrupting agent comprises a second targeting moiety or second effector moiety comprising a second CRISPR/Cas molecule comprising a second CRISPR/Cas protein and second guide RNA.
B162. The method or site-specific disrupting agent of any of the preceding embodiments, wherein the site-specific disrupting agent comprises a targeting moiety or effector moiety comprising TAL effector molecule, a CRISPR/Cas molecule, a zinc finger domain, a tetR domain, a meganuclease, or an oligonucleotide.
B163. The method or system of any of the preceding embodiments, wherein the first site-specific disrupting agent comprises a first targeting moiety or first effector moiety comprising TAL effector molecule, a CRISPR/Cas molecule, a zinc finger domain, a tetR domain, a meganuclease, or an oligonucleotide and the second site-specific disrupting agent comprises a second targeting moiety or second effector moiety comprising TAL effector molecule, a CRISPR/Cas molecule, a zinc finger domain, a tetR domain, a meganuclease, or an oligonucleotide.
B164. The method or site-specific disrupting agent of any of the preceding embodiments, wherein the site-specific disrupting agent comprises an effector moiety comprising a histone modifying functionality, e.g., a histone methyltransferase, histone demethylase, or histone deacetylase activity.
B165. The method or system of any of the preceding embodiments, wherein the first and/or the second site-specific disrupting agent comprises an effector moiety comprising a histone modifying functionality, e.g., a histone methyltransferase, histone demethylase, or histone deacetylase activity.
B166. The method, site-specific disrupting agent, or system of embodiment B164 or B165, wherein the effector moiety comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof.
B167. The method, site-specific disrupting agent, or system of embodiment B164 or B165, wherein the effector moiety comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.
B168. The method, site-specific disrupting agent, or system of embodiment B164 or B165, wherein the effector moiety comprises EZH2 or a functional variant or fragment of any thereof.
B169. The method, site-specific disrupting agent, or system of embodiment B164 or B165, wherein the effector moiety comprises HDAC8 or a functional variant or fragment of any thereof.
B170. The method or site-specific disrupting agent of any of the preceding embodiments, wherein the site-specific disrupting agent comprises an effector moiety comprising a DNA modifying functionality, e.g., a DNA methyltransferase.
B171. The method or system of any of the preceding embodiments, wherein the first and/or the second site-specific disrupting agent comprises an effector moiety comprising a DNA modifying functionality, e.g., a DNA methyltransferase.
B172. The method, site-specific disrupting agent, or system of embodiment B170 or B171, wherein the effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3l, or a functional variant or fragment of any thereof.
B173. The method, site-specific disrupting agent, or system of embodiment B170 or B171, wherein the effector moiety comprises MQ1 or a functional variant or fragment of any thereof.
B174. The method, site-specific disrupting agent, or system of embodiment B170 or B171, wherein the effector moiety comprises DNMT3 (e.g., DNMT3a, DNMT3L, DNMT3a/3l, DNMT3B1, DNMT3B2, DNMT3B3, DNMT3B4, DNMT3B5, or DNMT3B6) or a functional variant or fragment of any thereof.
B175. The method or site-specific disrupting agent of any of the preceding embodiments, wherein the site-specific disrupting agent comprises an effector moiety comprising a transcriptional repressor.
B176. The method or site-specific disrupting agent of any of the preceding embodiments, wherein the first and/or the second site-specific disrupting agent comprises an effector moiety comprising a transcriptional repressor.
B177. The method, site-specific disrupting agent, or system of embodiment B175 or B176, wherein the effector moiety comprises a protein chosen from KRAB, MeCP2, HP1, RBBP4, REST, FOG1, SUZ12, or a functional variant or fragment of any thereof.
B178. The method, site-specific disrupting agent, or system of embodiment B177, wherein the effector moiety comprises KRAB or a functional variant or fragment of any thereof.
B179. The method or site-specific disrupting agent of any of the preceding embodiments, wherein the site-specific disrupting agent comprises a polymer.
B180. The method or system of any of the preceding embodiments, wherein the first and/or the second site-specific disrupting agent comprises a polymer.
B181. The method, site-specific disrupting agent, or system of embodiment B179 or B180, wherein the polymer comprises a polyamide.
B182. The method, site-specific disrupting agent, or system of embodiment B179 or B180, wherein the polymer is an oligonucleotide.
B183. The method, site-specific disrupting agent, or system of embodiment B182, wherein the oligonucleotide has a sequence that comprises a complement of the first anchor sequence or to a sequence proximal to the first anchor sequence.
B184. The method, site-specific disrupting agent, or system of embodiment B882, wherein the oligonucleotide has a sequence that comprises a complement of the second anchor sequence or to a sequence proximal to the second anchor sequence.
B185. The method, site-specific disrupting agent, or system of any of embodiments B182-B184, wherein the oligonucleotide comprises a chemical modification.
B186. The method or site-specific disrupting agent, or system of embodiment B179 or B180, wherein the polymer is a peptide nucleic acid.
B187. The method, site-specific disrupting agent, or system of any preceding embodiment, wherein the site-specific disrupting agent comprises a peptide-nucleic acid mixmer.
B188. The method, site-specific disrupting agent, or system of any preceding embodiment wherein the site-specific disrupting agent (e.g., a targeting moiety or effector moiety of the site-specific disrupting agent) comprises a peptide or polypeptide.
B189. The method, site-specific disrupting agent, or system of embodiment B188, wherein the polypeptide is a zinc finger polypeptide.
B190. The method, site-specific disrupting agent, or system of embodiment B188, wherein the polypeptide is or comprises a Transcription activator-like effector nuclease (TALEN) polypeptide.
B191. The method or site-specific disrupting agent of any preceding embodiment, wherein the site-specific disrupting agent comprises a small molecule.
B192. The method or system of any preceding embodiment, wherein the first and/or the second site-specific disrupting agent comprises a small molecule.
B193. The method or site-specific disrupting agent of any of the preceding embodiments, wherein the site-specific disrupting agent further comprises an effector moiety, e.g., an epigenetic modifying agent, e.g., a DNA methyltransferase, histone deacetylase, or a histone methyltransferase.
B194. The method or system of any preceding embodiment, wherein the first and/or the second site-specific disrupting agent further comprises an effector moiety, e.g., an epigenetic modifying agent, e.g., a DNA methyltransferase, histone deacetylase, or a histone methyltransferase.
B195. The method or site-specific disrupting agent of any of the preceding embodiments, wherein the site-specific disrupting agent comprises a fusion molecule.
B196. The method or system of any preceding embodiments, wherein the first and/or the second site-specific disrupting agent comprises a fusion molecule.
B197. The method or site-specific disrupting agent of any preceding embodiments wherein the site-specific disrupting agent comprises a targeting moiety comprising a CRISPR/Cas molecule and an effector moiety comprising a transcriptional repressor, e.g., as a fusion molecule.
B198. The method or system of any preceding embodiment, wherein the first and/or the second site-specific disrupting agent comprises a targeting moiety comprising a CRISPR/Cas molecule and an effector moiety comprising a transcriptional repressor, e.g., as a fusion molecule.
B199. The method or site-specific disrupting agent of embodiment B198, wherein the targeting moiety comprises dCas9 and the effector moiety KRAB or a functional variant or portion thereof.
B200. The method or system of any preceding embodiment, wherein the first and/or the second targeting moiety comprises dCas9 and the effector moiety KRAB or a functional variant or portion thereof.
B201. The method or site-specific disrupting agent of any embodiments B1-B177, wherein the site-specific disrupting agent comprises a targeting moiety comprising a CRISPR/Cas molecule and an effector moiety comprising a histone methyltransferase, e.g., as a fusion molecule.
B202. The method or system of any preceding embodiment, wherein the first and/or the second site-specific disrupting agent comprises a targeting moiety comprising a CRISPR/Cas molecule and an effector moiety comprising a histone methyltransferase, e.g., as a fusion molecule.
B203. The method, site-specific disrupting agent, or system of embodiment B201, wherein the targeting moiety comprises dCas9 and the effector moiety comprises EZH2 or a functional variant or portion thereof.
B204. The method, site-specific disrupting agent, or system of any of embodiments B1-B196, wherein the site-specific disrupting agent comprises a targeting moiety comprising a CRISPR/Cas molecule and an effector moiety comprising a DNA methyltransferase, e.g., as a fusion molecule.
B205. The method, site-specific disrupting agent, or system of embodiment B204, wherein the targeting moiety comprises dCas9 and the effector moiety comprises MQ1 or a functional variant or portion thereof.
B206. The method, site-specific disrupting agent, or system of embodiment B203, wherein the targeting moiety comprises dCas9 and the effector moiety comprises DNMT3, e.g., DNMT3a/3l or a functional variant or portion thereof.
B207. The method, site-specific disrupting agent, or system of any of the preceding embodiments, wherein the site-specific disrupting agent comprises a targeting moiety comprising a CRISPR/Cas molecule, a first effector moiety comprising a histone methyltransferase, and a second effector moiety comprising a transcriptional repressor, e.g., as a fusion molecule.
B208. The method, site-specific disrupting agent, or system of embodiment B207, wherein the targeting moiety comprises dCas9, the first effector moiety comprises EZH2 or a functional variant or portion thereof, and the second effector moiety comprises KRAB or a functional variant or portion thereof.
B209. The method, site-specific disrupting agent, or system of any of the preceding embodiments, wherein the site-specific disrupting agent comprises a targeting moiety comprising a CRISPR/Cas molecule, and an effector moiety comprising a histone deacetylase, e.g., as a fusion molecule.
B210. The method, site-specific disrupting agent, or system of embodiment B209, wherein the targeting moiety comprises dCas9 and the effector moiety comprises HDAC8 or a functional variant or portion thereof.
B211. The method, site-specific disrupting agent, or system of any of the preceding embodiments, wherein the site-specific disrupting agent comprises a targeting moiety comprising a CRISPR/Cas molecule, a first effector moiety comprising a histone methyltransferase, and a second effector moiety comprising a histone deacetylase, e.g., as a fusion molecule.
B212. The method, site-specific disrupting agent, or system of embodiment B211, wherein the targeting moiety comprises dCas9, the first effector moiety comprises EZH2 or a functional variant or portion thereof, and the second effector moiety comprises HDAC8 or a functional variant or portion thereof.
B213. The method, site-specific disrupting agent, or system of any of embodiments B195-B212, wherein the site-specific disrupting agent comprises an amino acid sequence encoded by a nucleic acid sequence chosen from SEQ ID NOs: 69, 71, 85, 201, 202, 204, 205, 207, 209, 211, 213, 215, 217, or 219-242, a complementary or reverse complementary sequence of any thereof, or comprises a sequence with at least 80, 85, 90, 91, 92, 93, 94, 95, 96, 97, 98, or 99% identity to any thereof.
B214. The method, site-specific disrupting agent, or system of any of embodiments B195-B213, wherein the site-specific disrupting agent comprises an amino acid sequence chosen from any one of SEQ ID NOs:70, 72, 82, 84, 86, 203, 206, 208, 210, 212, 214, 216, or 218, or encoded by a sequence chosen from any one of SEQ ID NOs: 219-242, or comprises a sequence with at least 80, 85, 90, 91, 92, 93, 94, 95, 96, 97, 98, or 99% identity to any thereof.
B215. The method, site-specific disrupting agent, or system of any of the preceding embodiments, wherein the cell is situated in a subject.
B216. The method, site-specific disrupting agent of any of embodiments B1-B215, wherein the cell is ex vivo.
B217. The method or site-specific disrupting agent, or system of any of the preceding embodiments, wherein the cell is a mammalian cell, e.g., a human cell.
B218. The method, site-specific disrupting agent, or system of any of the preceding embodiments, wherein the cell is a somatic cell.
B219. The method, site-specific disrupting agent, or system of any of the preceding embodiments, wherein the cell is a primary cell.
B220. The method of any of the preceding embodiments, wherein the step of contacting is performed ex vivo.
B221. The method of embodiment B220, further comprising, prior to the step of contacting, a step of removing the cell (e.g., mammalian cell) from a subject.
B222. The method of either of embodiments B220 or B221, wherein further comprising, after the step of contacting, a step of (b) administering the cells (e.g., mammalian cells) to a subject.
B223. The method of any of embodiments B1-B222, wherein the step of contacting comprises administering a composition comprising the site-specific disrupting agent to a subject.
B224. The method of embodiment B223, wherein the site-specific disrupting agent is administered as a monotherapy.
B225. The method of embodiment B223, wherein the site-specific disrupting agent is administered in combination with a second therapeutic agent.
B226. A reaction mixture comprising a cell (e.g., a human cell, e.g., a primary human cell) and a site-specific disrupting agent, or system of any of preceding embodiments.
B227. A method of treating a subject having an inflammatory disorder, comprising: administering to the subject a site-specific disrupting agent, system or reaction mixture of any preceding embodiments in an amount sufficient to treat the inflammatory disorder, thereby treating the inflammatory disorder.
B228. The method of embodiment B227, wherein the inflammatory disorder is rheumatoid arthritis, psoriasis, or inflammatory bowel disease.
B229. The method of embodiment B227 or B228, wherein the inflammatory disorder is rheumatoid arthritis, gout, neutrophilic asthma, neutrophilic dermatosis, acute respiratory disease syndrome (ARDS), or COVID-19.
B230. The method of any of embodiments B227-B229, wherein the inflammatory disorder is an autoimmune disorder, e.g., rheumatoid arthritis.
B231. The method of any of embodiments B227-B229, wherein the inflammatory disease is associated with a pathogenic infection, e.g., viral infection, e.g., SARS-CoV2 infection.
B232. The method of any of embodiments B227-B229, wherein the inflammatory disease is associated with a superinfection, e.g., infection caused by two or more pathogenic agents, e.g., by a virus and a bacterium, (e.g., by SARS-CoV2 and Streptococcus pneumoni), e.g., by a virus and a fungus, (e.g., by SARS-CoV2 and mucormycosis).
B232. A method of treating a subject having cancer, comprising:
   administering to the subject an expression repressor, system, nucleic acid, nucleic acid system, or reaction mixture of any of the preceding embodiments in an amount sufficient to treat the cancer,
thereby treating the cancer.
B233. The method of claim B232, wherein the cancer is lung cancer (e.g., non-small cell lung cancer), breast cancer, hepatocellular carcinoma (HCC), prostate cancer, colon cancer, skin cancer, cervical cancer, ovarian cancer, uterine endometrioid carcinoma, endometrial cancer, mature B-cell lymphoma, bladder cancer, esophagogastric cancer, esophageal adenocarcinoma, bone cancer, melanoma, hepatobiliary cancer, thyroid cancer, mature B-cell neoplasms, glioma, head-neck squamous cell carcinoma, kidney renal clear cell carcinoma, pancreatic cancer (e.g., pancreatic ductal adenocarcinoma), sarcoma, or stomach adenocarcinoma.

Those skilled in the art will recognize or be able to ascertain using no more than routine experimentation, many equivalents to the specific embodiments of the disclosure described herein.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Mar. 30, 2022. When one gene or protein references a plurality of sequence accession numbers, all of the sequence variants are encompassed.

### Definitions

A, an, the: As used herein, the singular forns “a,” “an” and “the” include plural referents unless the context clearly dictates otherwise.

Anchor Sequence: The term “anchor sequence” as used herein, refers to a nucleic acid sequence recognized by a nucleating agent that binds sufficiently to form an anchor sequence-mediated conjunction, e.g., a complex. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction, or insulating against an enhancer that is outside the anchor sequence mediated conjunction. In some embodiments of the present disclosure, technologies are provided that may specifically target a particular anchor sequence or anchor sequences, without targeting other anchor sequences (e.g., sequences that may contain a nucleating agent (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule—e.g., the same chromosome) as the targeted anchor sequence is not modulated. In some embodiments, the anchor sequence comprises or is a nucleating polypeptide binding motif. In some embodiments, the anchor sequence is adjacent to a nucleating polypeptide binding motif.

Anchor Sequence-Mediated Conjunction: The term “anchor sequence-mediated conjunction” as used herein, refers to a DNA structure, in some cases, a complex, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequences.

Associated with: Two events or entities are “associated” with one another, as that term is used herein, if presence, level, form and/or function of one is correlated with that of the other. For example, in some embodiments, a particular entity (e.g., polypeptide, genetic signature, metabolite, microbe, etc.) is considered to be associated with a particular disease, disorder, or condition, if its presence, level, form and/or function correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, a DNA sequence is “associated with” a target genomic or transcription complex when the nucleic acid is at least partially within the target genomic or transcription complex, and expression of a gene in the DNA sequence is affected by formation or disruption of the target genomic or transcription complex.

CXCL locus: As used herein, the term “CXCL locus” refers to the portion of the human genome that encodes CXCL1-7 and IL-8, enhancers E1 and E2, and anchor sequences that form an ASMC comprising CXCL1-7 and IL-8, or the homologous region of the genome in a non-human animal. In some embodiments, the CXCL locus is situated on human chromosome 4.

CXCL gene: As used herein, the term “CXCL gene” refers to human CXCL1, CXCL2, CXCL3, CXCL4, CXCL5, CXCL6, CXCL7, or IL-8, or a homologous non-human gene. Human IL-8 is sometimes also referred to as CXCL8.

Site-specific disrupting agent: As used herein, the term “site-specific disrupting agent” refers to an agent or entity that specifically inhibits, dissociates, degrades, and/or modifies one or more components of a genomic complex, e.g., ASMC, thereby modulating, e.g., decreasing, expression of a target plurality of genes as described herein. In some embodiments, a site-specific disrupting agent interacts with one or more components of a genomic complex. In some embodiments, a site-specific disrupting agent binds (e.g., directly or, in some embodiments, indirectly) to one or more genomic complex components. In some embodiments, a site-specific disrupting agent binds to an anchor sequence, e.g., a first and/or second anchor sequence, that may be part of an ASMC comprising a target plurality of genes. In some embodiments, a site-specific disrupting agent binds to a site proximal to an anchor sequence, e.g., a first and/or second anchor sequence, that may be part of an ASMC comprising a target plurality of genes. In some embodiments, a site-specific disrupting agent modifies one or more genomic complex components. In some embodiments, a site-specific disrupting agent comprises an oligonucleotide. In some embodiments, a site-specific disrupting agent comprises a polypeptide. In some embodiments, a site-specific disrupting agent comprises an antibody (e.g., a monospecific or multi-specific antibody construct) or antibody fragment. In some embodiments, a site-specific disrupting agent is directed to a particular genomic location and/or to a genomic complex by a targeting moiety, as described herein. In some embodiments, a site-specific disrupting agent comprises a genomic complex component or variant thereof. In some embodiments, a site-specific disrupting agent comprises a targeting moiety. In some embodiments, a site-specific disrupting agent comprises an effector moiety. In some embodiments, a site-specific disrupting agent comprises a plurality of effector moieties. In some embodiments, a site-specific disrupting agent comprises a targeting moiety and one or more effector moieties. In some embodiments, the site-specific disrupting agent specifically binds a first site in the genome with higher affinity than a second site in the genome (e.g., relative to any other site in the genome). In some embodiments, the site-specific disrupting agent preferentially inhibits, dissociates, degrades, and/or modifies one or more components of a first genomic complex relative to a second genomic complex (e.g., relative to any other genomic complex). In some embodiments, a site-specific disrupting agent may be an expression repressor, e.g., the site-specific disrupting agent may inhibit an ASMC, thereby reduce expression of a gene in the ASMC.

Domain: As used herein, the term “domain” refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively, or additionally, in some embodiments, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is or comprises a section or portion of a molecule (e.g., a small molecule, carbohydrate, lipid, nucleic acid, polypeptide, etc.). In some embodiments, a domain is or comprises a section of a polypeptide. In some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).

E1 cis-acting regulatory element (E1 cRE): The term “E1 cRE” and “E1 cis-acting regulatory element), as used herein, refers to a nucleic acid sequence positioned proximal to (e.g., approximately 14 kb upstream of) IL8 in the human genome (see FIG. 16B) and recognized by a trans-acting factor (e.g., a transcription factor, e.g., p65) that binds sufficiently to upregulate expression of one or more CXCL genes.

E2 cis-acting regulatory element (E2 cRE): The term “E2 cRE” and “E2 cis-acting regulatory element), as used herein, refers to a nucleic acid sequence positioned proximal to CXCL2 in the human genome (see FIG. 16B) and recognized by a trans-acting factor (e.g., a transcription factor, e.g., p65) that binds sufficiently to upregulate expression of one or more CXCL genes.

Effector moiety: As used herein, the term “effector moiety” refers to a domain with one or more functionalities that modulate, e.g., decrease, expression of a target plurality of genes in a cell when appropriately localized in the nucleus of a cell. In some embodiments, an effector moiety comprises a polypeptide. In some embodiments, an effector moiety comprises a polypeptide and a nucleic acid. A functionality associated with an effector moiety may directly affect expression of a target plurality of genes, e.g., blocking recruitment of a transcription factor that would stimulate expression of the gene. A functionality associated with an effector moiety may indirectly affect expression of a target plurality of genes, e.g., introducing epigenetic modifications or recruiting other factors that introduce epigenetic modifications that induce a change in chromosomal topology that inhibits expression of a target plurality of genes.

Expression repressor: As used herein, the term “expression repressor” refers to an agent or entity with one or more fumctionalities that decreases expression of a target gene in a cell and that specifically binds to a DNA sequence (e.g., a DNA sequence associated with a target gene or a transcription control element operably linked to a target gene). An expression repressor comprises at least one targeting moiety and optionally one effector moiety. In some embodiments, an expression repressor binds to a site proximal to an enhancer sequence that may be operably linked to a target plurality of genes. In some embodiments, an expression repressor comprises an oligonucleotide. In some embodiments, an expression repressor comprises a polypeptide. In some embodiments, an expression repressor comprises a plurality of effector moieties. In some embodiments, an expression repressor comprises a targeting moiety and one or more effector moieties. In some embodiments, the expression repressor specifically binds a first site in the genome with higher affinity than a second site in the genome (e.g., relative to any other site in the genome).

Genomic complex: As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex binds. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory site (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., eRNA), transcriptional machinery proteins (e.g., RNA polymerase, one or more transcription factors, for example selected from the group consisting of TFIA, TFIIB, TFTID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration (e.g., a loop) that it does not adopt when the complex is not formed.

Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively, or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity. In some embodiments, a nucleic acid is an mRNA nucleic acid and may be monocistronic or polycistronic (e.g., bi-cistronic, tri-cistronic, etc.).

Operably linked: As used herein, the phrase “operably linked” refers to a juxtaposition wherein the components described are in a relationship permitting them to function in their intended manner. A genomic regulatory element (e.g., transcription control element) “operably linked” to a functional element, e.g., gene, is associated in such a way that expression and/or activity of the functional element, e.g., gene, is achieved under conditions compatible with the genomic regulatory element (e.g., transcription control element). In some embodiments, an “operably linked” genomic regulatory element (e.g., transcription control elements) is contiguous (e.g., covalently linked) with coding elements, e.g., genes, of interest; in some embodiments, operably linked an genomic regulatory element (e.g., transcription control elements) acts in cis to or otherwise at a distance from the functional element, e.g., gene, of interest. In some embodiments, an “operably linked” genomic regulatory element (e.g., transcription control element) is contiguous (e.g., covalently linked) with a coding element, e.g., gene, of interest; in some embodiments, an operably linked genomic regulatory element (e.g., transcription control element) acts in trans to or otherwise at a distance from the functional element, e.g., gene, of interest. In some embodiments, two operably linked nucleic acid sequences are comprised on the same nucleic acid.

In a further embodiment, two operably linked nucleic acid sequences are proximal to one another on the same nucleic acid, e.g., within 1000, 500, 100, 50, or 10 base pairs of each other or directly adjacent to each other.

Peptide, Polypeptide, Protein: As used herein, the terms “peptide,” “polypeptide,” and “protein” refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence.

Polypeptides include any peptide or protein comprising two or more amino acids joined to each other by peptide bonds or by means other than peptide bonds. As used herein, the term refers to both short chains, which also commonly are referred to in the art as peptides or oligopeptides, for example, and to longer chains, which generally are referred to in the art as proteins, of which there are many types.

Proximal: As used herein, “proximal” refers to a closeness of two sites, e.g., nucleic acid sites, such that binding of an expression repressor or site-specific disrupting agent at the first site and/or modification of the first site by an expression repressor or site-specific disrupting agent will produce the same or substantially the same effect as binding and/or modification of the other site. For example, a DNA-targeting moiety may bind to a first site that is proximal to an anchor sequence (the second site), and the effector moiety associated with said DNA-targeting moiety may epigenetically modify the first site such that the binding of the anchor sequence to an endogenous nucleating polypeptide modified, substantially the same as if the second site (the anchor sequence) had been bound and/or modified. In some embodiments, sites that are proximal to one another are less than 5000, 4000, 3000, 2000, 1000, 900, 800, 700, 600, 500, 400, 300, 200, 100, 50, 40, 30, 20, 10, or 5 base pairs from one another.

Sequence targeting polypeptide: As used herein, the term “sequence targeting polypeptide” as used herein, refers to a protein, e.g., a protein comprising a CRISPR/Cas domain, a TAL effector domain, or a Zn Finger domain, that recognizes or specifically binds to a target nucleic acid sequence. In some embodiments, the sequence targeting polypeptide is a catalytically inactive protein, such as dCas9, a TAL effector molecule, or a Zn Finger domain, that lacks endonuclease activity.

Specific binding: As used herein, the term “specific binding” refers to an ability to discriminate between possible binding partners in the environment in which binding is to occur. In some embodiments, a binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex. In some embodiments, specific binding is assessed by detecting or determining ability of the binding agent to compete with an alternative interaction between its partner and another entity. In some embodiments, specific binding is assessed by performing such detections or determinations across a range of concentrations.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

Symptoms are reduced: As used herein, the phrase “symptoms are reduced” may be used when one or more symptoms of a particular disease, disorder or condition is reduced in magnitude (e.g., intensity, severity, etc.) and/or frequency. In some embodiments, a delay in the onset of a particular symptom is considered one form of reducing the frequency of that symptom.

Target: An agent or entity is considered to “target” another agent or entity, in accordance with the present disclosure, if it binds specifically to the targeted agent or entity under conditions in which they come into contact with one another. In some embodiments, for example, an antibody (or antigen-binding fragment thereof) targets its cognate epitope or antigen. In some embodiments, a nucleic acid having a particular sequence targets a nucleic acid of substantially complementary sequence. In some embodiments, a targeting moiety that specifically binds an anchor sequence targets the anchor sequence, the ASMC comprising the anchor sequence, and/or the plurality of genes within the ASMC.

Target plurality of genes: As used herein, the term “target plurality of genes” means a group of more than one gene (e.g., 2, 3, 4, 5, 6, 7, 8, 9, or more genes) that is targeted for modulation, e.g., of expression. In some embodiments, a target plurality of genes is part of a targeted genomic complex. In some embodiments, each gene of a target plurality of genes is operably linked to an enhancer, e.g., an E1 enhancer, wherein the enhancer is targeted by an expression repressor as described herein. In some embodiments, modulation comprises inhibition of expression of the target plurality of genes. In some embodiments, a target plurality of genes is modulated by contacting the target plurality of genes or a genomic regulatory element (e.g., transcription control element) operably linked to one or more of the target plurality of genes with an expression repressor described herein. In some embodiments, one or more of a target plurality of genes is aberrantly expressed (e.g., over-expressed) in a cell, e.g., a cell in a subject (e.g., patient). In some embodiments, the target plurality of genes has related functionalities. For example, the genes of a target plurality of genes may all have a pro-inflammatory effect when expressed; the genes of such a target plurality of genes may be referred to herein as pro-inflammatory genes or target pro-inflammatory genes. In some embodiments, a gene of a target plurality of genes encodes a protein. In some embodiments, a gene of a target plurality of genes encodes a functional RNA.

Targeting moiety: As used herein, the term “targeting moiety” means an agent or entity that specifically interacts (e.g., targets) with a component or set of components, e.g., DNA. In some embodiments the component or components participates in a genomic complex as described herein (e.g., an anchor sequence-mediated conjunction). In some embodiments, a targeting moiety in accordance with the present disclosure targets one or more target component(s) of a genomic complex as described herein. In some embodiments, a targeting moiety targets a genomic regulatory element (e.g., an E1 enhancer). In some embodiments, a targeting moiety targets an anchor sequence. In some embodiments, a targeting moiety targets a genomic complex component other than a genomic regulatory element. In some embodiments, a targeting moiety targets a plurality or combination of genomic complex components, which plurality in some embodiments may include a genomic sequence element. In some aspects, effective inhibition, dissociation, degradation, and/or modification of one or more genomic complexes, as described herein, can be achieved by targeting complex component(s) comprising genomic sequence element(s). In some embodiments, the present disclosure contemplates that improved (e.g., with respect to, for example, degree of specificity for a particular genomic complex as compared with other genomic complexes that may form or be present in a given system, effectiveness of the inhibition, dissociation, degradation, or modification [e.g., in terms of impact on number of complexes detected in a population]) inhibition, dissociation, degradation, or modification may be achieved by targeting one or more complex components that is not a genomic sequence element and, optionally, may alternatively or additionally include targeting a genomic sequence element, wherein improved inhibition, dissociation, degradation, or modification is relative to that typically achieved through targeting genomic sequence element(s) alone.

In some embodiments, a site-specific disrupting agent as described herein promotes inhibition, dissociation, degradation, or modification of a target genomic complex. For example, by way of non-limiting example, in some embodiments, a site-specific disrupting agent as described herein inhibits, dissociates, degrades (e.g., a component of), and/or modifies (e.g., a component of) an anchor sequence-mediated conjunction by targeting at least one component of a given genomic complex (e.g., comprising the anchor sequence-mediated conjunction). In some embodiments, a site-specific disrupting agent as described herein inhibits, dissociates, degrades (e.g., a component of), and/or modifies (e.g., a component of) a particular genomic complex (i.e., a target genomic complex) and does not inhibit, dissociate, degrade (e.g., a component of), and/or modify (e.g., a component of) at least one other particular genomic complex (i.e., a non-target genomic complex) that, for example, may be present in other cells (e.g., in non-target cells) and/or that may be present at a different site in the same cell (i.e., within a target cell). An expression repressor or a site-specific disrupting agent as described herein may comprise a targeting moiety. In some embodiments, a targeting moiety also acts as an effector moiety (e.g., disrupting moiety); in some such embodiments a provided expression repressor or site-specific disrupting agent may lack any effector moiety (e.g., disrupting, modifying, or other effector moiety) separate (or meaningfully distinct) from the targeting moiety.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as desired biological endpoint(s), substance to be delivered, target cell(s) ortissue(s), etc. For example, in some embodiments, an effective amount of compound in a formulation to treat a disease, disorder, and/or condition is an amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Genomic regulatory sequence: As used herein, the term “genomic regulatory sequence” refers to a nucleic acid sequence that increases or decreases transcription of a gene. An “enhancing sequence” increases the likelihood of gene transcription. A “silencing or repressor sequence” decreases the likelihood of gene transcription. Examples of genomic regulatory sequences include promoters and enhancers. In some embodiments, the genomic regulatory sequence is a cis-acting regulatory element. In some embodiments, an ASMC comprises a genomic regulatory sequence. Such a genomic regulatory sequence is referred to as an internal genomic regulatory sequence (e.g., an enhancing sequence that is comprised within an ASMC is referred to as an internal enhancing sequence).

### BRIEF DESCRIPTION OF THE DRAWINGS

The following detailed description of the embodiments of the disclosure will be better understood when read in conjunction with the appended drawings. For the purpose of illustrating the disclosure, there are shown in the drawings embodiments, which are presently exemplified. It should be understood, however, that the disclosure is not limited to the precise arrangement and instrumentalities of the embodiments shown in the drawings.

FIG. 1 shows a diagram showing exemplary positioning of gRNA sequences in an anchor sequence. FIG. 1 discloses SEQ ID NOS 244-245, respectively, in order of appearance.

FIG. 2 shows a diagram showing exemplary positioning of gRNA sequences in an anchor sequence and restriction site information. FIG. 2 discloses SEQ ID NOS 246-247, respectively, in order of appearance.

FIG. 3 shows a graph of expression (mRNA) of various chemokines in TNF-treated cells with and without treatment with a site-specific disrupting agent comprising a CRISPR/Cas molecule and first exemplary gRNA.

FIG. 4 shows a graph of expression (mRNA) of various chemokines in TNF-treated cells with and without treatment with a site-specific disrupting agent comprising a CRISPR/Cas molecule and a second exemplary gRNA.

FIG. 5 shows a diagram depicting different types of genomic complex, e.g., ASMCs, e.g., loops, and models for how to alter expression of genes contained within.

FIG. 6 shows a graph of cytokine expression measured by RNA levels of CXCL1, CXCL2, CXCL3, and IL-8 in THP-1 cells treated with a site-specific disrupting agent comprising a CRISPR/Cas molecule and sgRNAs targeted to the anchor sequences of a genomic complex (e.g., ASMC) comprising cytokine-encoding genes.

FIG. 7 shows a graph of cytokine secretion (CXCL1 and IL-8) of THP-1 cells treated with site-specific disrupting agent comprising a CRISPR/Cas molecule and different sgRNAs targeted to the anchor sequences of a genomic complex (e.g., ASMC) comprising cytokine-encoding genes.

FIG. 8 shows a graph (top) of cytokine expression (CXCL3) measured by RNA level in THP-1 cells treated with a site-specific disrupting agent comprising a CRISPR/Cas molecule and sgRNAs targeted to the anchor sequences of a genomic complex (e.g., ASMC) comprising cytokine-encoding genes, and a flow chart (bottom) showing how cells were processed in the experiment.

FIG. 9A shows a graph (top) of cytokine expression (CXCL1) measured by RNA level in THP-1 cells 3 weeks after treatment with a site-specific disrupting agent comprising a CRISPR/Cas molecule and sgRNAs targeted to the anchor sequences of a genomic complex (e.g., ASMC) comprising cytokine-encoding genes, and a flow chart (bottom) showing how cells were processed in the experiment. FIG. 9B shows a graph of cytokine expression (CXCL3) measured by RNA level in THP-1 cells 3 weeks after treatment with a site-specific disrupting agent comprising a CRISPR/Cas molecule and sgRNAs targeted to the anchor sequences of a genomic complex (e.g., ASMC) comprising cytokine-encoding genes.

FIG. 10 shows a graph of cytokine expression (CXCL1) measured by RNA level in THP-1 cells after treatment with a site-specific disrupting agent comprising a catalytically inactive CRISPR/Cas molecule and a transcriptional repressor (KRAB) and sgRNAs targeted to the anchor sequences of a genomic complex (e.g., ASMC) comprising cytokine-encoding genes.

FIG. 11 shows a graph of cytokine expression (CXCL1) measured by RNA level in THP-1 cells after treatment with a site-specific disrupting agent comprising a catalytically inactive CRISPR/Cas molecule and a histone methyltransferase (EZH2) and sgRNAs targeted to the anchor sequences of a genomic complex (e.g., ASMC) comprising cytokine-encoding genes.

FIG. 12 shows a graph of cytokine expression (CXCL1) measured by RNA level in THP-1 cells after treatment with a site-specific disrupting agent comprising a catalytically inactive CRISPR/Cas molecule and a DNA methyltransferase (MQ1) and sgRNAs targeted to the anchor sequences of a genomic complex (e.g., ASMC) comprising cytokine-encoding genes.

FIG. 13 shows a graph (top) of cytokine expression (CXCL1) measured by RNA level in THP-1 cells after treatment with different site-specific disrupting agents for 72 hours, 3 weeks, or 4 weeks, and a flow chart (bottom) showing how cells were processed in the experiment.

FIG. 14 shows a graph (top) of cytokine expression (CXCL3) measured by RNA level in THP-1 cells after treatment with different site-specific disrupting agents and sgRNAs targeted to the anchor sequences of a genomic complex (e.g., ASMC) comprising cytokine-encoding genes, and a flow chart (bottom) showing how cells were processed in the experiment.

FIG. 15 shows a graph (top) of cytokine expression (CXCL1) measured by RNA level in THP-1 cells after treatment with different site-specific disrupting agents and sgRNAs targeted to the anchor sequences of a genomic complex (e.g., ASMC) comprising cytokine-encoding genes.

FIG. 16 shows human CXCL IGD and gene cluster organization. FIG. 16A shows a schematic Insulated Genomic Domain (IGD) illustrating the two loops within CXCL1-8 gene cluster. CXCL8, CXCL6, and CXCL1 genes reside on the left loop of the IGD. CXCL2-5 and CXCL7 genes reside on the right loop of the IGD. Investigation of the IGD data from different cell lines suggested that middle CTCF is only present in cells that secrete CXCL (e.g., not in lymphocytes). FIG. 16B shows guides were designed to the four different CTCF targets: Left CTCF-2, Left CTCF, Middle CTCF, and Right CTCF.

FIG. 17 shows CXCL1-8 genes were downregulated when dCas9-EZH2 guide 30183 targeted Middle CTCF motif located within the CXCL1-8 cluster in TNF-alpha treated Human A549 lung cancer epithelial cells. Cells stimulated with TNF alpha were treated as control.

FIG. 18 shows CXCL1, 2, 3, 8 genes were downregulated when dCas9-EZH2 guide 30183 targeted Middle CTCF motif located within the CXCL1-8 cluster in TNF-alpha treated Human IMR-90 normal lung fibroblast cells. Cells stimulated with TNF alpha were treated as control.

FIG. 19 shows that CXCL1, 2, 3, 8 genes were downregulated when Controller A targeted Left CTCF motif located within the CXCL1-8 cluster in TNF-alpha treated Human monocytes. Cells stimulated with TNF alpha were treated as control.

FIG. 20 shows mouse CXCL IGD and gene cluster organization. FIG. 20A shows a schematic Insulated Genomic Domain (IGD) illustrating the two loops within CXCL gene cluster. FIG. 20B illustrates the two loops within the CXCL1-5, 7 and 15 gene cluster. CXCL4, CXCL5, and CXCL7 genes reside on the left loop of the IGD. CXCL1-3 and CXCL15 genes reside on the right loop of the IGD guides were designed to the four different CTCF targets: Left (L), Middle 1 (M1), Middle 2 (M2), and Right (R) CTCF.

FIG. 21A shows IGD guides were designed to the four different CTCF targets: Middle 1 (M1), Middle 2 (M2), and Right (R) CTCF.

FIG. 21B shows in vitro downregulation of mouse CXCL IGD in Hep 1.6 using dCas9-MQ1. dCas9-MQ1 was transfected using guides targeting the right, or one of the two middle CTCF motifs in the CXCL gene cluster, which showed no down regulation in any of the seven CXCL genes after TNF alpha stimulation (orange). When dCas9-MQ1 was transfected using combination guides targeting both middle CTCF and right, the entire gene cluster was down regulated (blue).

FIG. 22A shows schematic experimental design to determine the effect of dCas9-MQ1 on decreasing leukocyte filtration in inflamed lungs. Each mouse was treated with either LNP alone or with dCas9-MQ1 at 3 mg/kg targeting the two middle and right CTCF at −2 hour time point. The mice were simulated with 5 mg/kg LPS at zero hours followed by a second dose of LNP alone or a dCas9-MQ1 at 3 mg/kg targeting the two middle and right CTCF at the +8 hour time point. Dexamethasone was administered intraperitoneal at 10 mg/kg dose at time 0, 24, and 48 hours. The animals were terminated at 72 hours and bronchiolar lavage fluid were collected from the lungs for flow staining.

FIG. 22B shows systemic administration of a dCas9-MQ1 decreased leukocyte infiltration in the inflamed lungs. Total leukocyte count/mL in the bronchiolar lavage fluid obtained from dCas9-MQ1 treated mice showed significant differences compared to LPS+disease animals.

FIG. 23A shows the composition of infiltrating cells found in the bronchiolar lavage fluid obtained from an inflamed lung of a mice. The leukocyte cell types that make up the majority of the infiltrating cells are neutrophils, followed by B cells, T cells, macrophages and other types of hematopoietic cells.

FIG. 23B shows dCas9-MQ1 decreased the count of neutrophils infiltrating the lungs with significant difference compared to the +LPS disease group.

FIG. 24 shows the decrease of leukocyte cells in the BALF was lung specific and not due to the decrease of white blood cells in the peripheral blood. This graph illustrated that the effect of decreasing leukocyte count in the BALF with the dCas9-MQ1 treatment was lung specific and was not because the mouse itself had a decrease in leukocyte population. The hematopoietic cell population in the peripheral blood was similar across all groups.

FIGS. 25A-G show CXCL1-5, CXCL7, and CXCL15 gene expression was decreased in the lung tissue. After treating the animals with LNP alone or with dCas9-MQ1, the lung tissues were processed to check for CXCL gene expression by qPCR methods. All CXCL genes show downregulation when treated with dCA9-MQ1. CXCL2 expression was most downregulated.

FIGS. 26A-D show decreasing CXCL expression and cellular recruitment to the site of inflammation had a beneficial downstream effect of decreasing the presence of other cytokines. The chemokine protein levels secreted in the BALF showed decrease in CXCL1 and 2 protein levels. Decreasing CXCL expression and cellular recruitment to the site of inflammation had beneficial downstream effects of decreasing the presence of GM-CSF (FIG. 26C) and IL6 (FIG. 26D).

FIGS. 27 and 28 are bar graphs showing the % downregulation (vs. cells+IL-1A) of CXCL genes using expression repressors targeting different sites in an E1 cRE. Overall, these graphs show how numerous effectors targeted to two different sites in the E1 cRE are able to achieve downregulation of multiple genes near the E1 cRE.

FIG. 29 is a bar graph showing the % downregulation (vs. cells+IL-1A) of CXCL genes using expression repressors targeting a site in an E2 cRE.

FIGS. 30 and 31 are bar graphs showing how dCas9-KRAB (FIG. 30) and dCas9-MQ1 (FIG. 31) targeting a site in an E1 cRE are able to achieve downregulation of multiple genes near the E1 cRE. *p<0.05, ***p<0.001, ****p<0.0001 FIGS. 32 and 33 are bar graphs showing how dCas9-KRAB (FIG. 32) and dCas9-MQ1 (FIG. 33) targeting a site in an E1 cRE are able to achieve downregulation of multiple genes near the E1 cRE. *p<0.05, ***p<0.001, ****p<0.0001 FIG. 34 is a bar graph showing how an expression repressor (dCas9-KRAB) targeting the IL8 promoter successfully downregulates IL8 expression.

FIG. 35 is a bar graph showing how two expression repressors comprising zinc finger domain targeting moieties directed to different sites in the E1 cRE are able to achieve downregulation of multiple genes near the E1 cRE. Furthermore, the graph shows a dCas9-KRAB expression repressor directed to the IL8 promoter decreased expression of IL8 greater than 90%.

FIG. 36 is a bar graph showing a E1 cRE targeting expression repressor (zinc finger-KRAB), an IL8 promoter targeting expression repressor (dCas9-KRAB), and a combination of the two, do not interfere with one another and that the combination of expression repressors has a greater effect on IL8 compared to either expression repressor alone.

FIG. 37 is a bar graph showing decreasing expression of IL8 using expression repressors targeting a site in the E1 cRE or the IL8 promoter as measured by IL8 mRNA one hour after IL1A stimulation.

FIGS. 38 and 39 are bar graphs showing decreasing expression of IL8 using expression repressor targeting as site in the E1 cRE or the IL8 promoter, where IL8 protein levels are measured by ELISA at 6 hours (FIG. 38) and 24 hours (FIG. 39) after IL1A stimulation.

FIG. 40 is a bar graph depicting the downregulation of mRNA levels of CXCL1-3 and IL8 (percent downregulation calculated with normalization to IL1A treated control) by two expression repressors directed to two sites in the E1 cRE.

FIG. 41 is a bar graph showing the ability of two expression repressors (MR32105 and MR32104 comprising zinc finger targeting moieties and KRAB effector domains) directed to two sites in the E1 cRE to increase H3Kme3 as measured ChIP qPCR.

FIG. 42 is a bar graph showing the downregulation of CXCL1-3 and IL8 at 3-7 days post introduction of an expression repressor (MR32105) targeting the E1 cRE. Percent CXCL1-3 and IL8 gene downregulation was calculated with normalization to IL-1A treated control. Downregulation of CXCL1, CXCL2, CXCL3, and IL8 are shown in order from left to right in groups of Day 3-7.

FIG. 43 is a bar graph showing the downregulation of IL8 using expression repressors targeting different sites in the IL8 promotor. Overall, this graph shows how numerous effectors targeted to different sites in the IL8 promotor are able to achieve downregulation of IL8.

FIGS. 44A and 44B shows enrichment of E1-targeting expression repressor derived from MR-32105 to the E1 site (top panel), the resultant increase in on-target DNA histone methylation (H3K9me3) (middle panels) and decrease in on-target histone acetylation (H3K27ac) (bottom panels) (FIG. 44A). FIG. 44B shows a depletion of the P65 transcription factor at the E1 locus resulting from the expression repressor according to MR-32105.

FIG. 45 is a bar graph showing the downregulation of CXCL1-3 and IL8 relative to 1 hr IL1A stimulation after introduction of an expression repressor (MR-32104 or MR-32105) targeting the E1 cRE.

FIGS. 46A and 46B are box and whisker blots showing CXCL gene downregulation after introduction of an expression repressor (MR-32104 and MR-32105) targeting the E1 cRE.

FIG. 47 shows enrichment of IL8-targeting expression repressor derived from MR-32712 at the target IL8 (top panel), the resultant increase in on-target DNA histone methylation (H3K9me3) (middle panels) and decrease in on-target P65 binding (bottom panels) by HA-ChIP Seq.

FIG. 48 is a bar graph showing CXCL gene expression in IMR-90 cells after an IL8 targeting expression repressor (MR-32712).

FIG. 49 shows box and whisker plots showing RNA levels for CXCL gene expression after introduction of an IL8-targeting expression repressor (MR-32172). Overall, the whisker plots show significant decrease of the IL8 RNA.

FIG. 50 shows enrichment of E1-targeting expression repressor at 24 hours but no detectable signal at 24 hours by HA-ChIP Seq.

FIG. 51 are bar graphs showing the CXCL gene and protein downregulation in small airway epithelial cells (COPD) after introduction of an expression repressor targeting TL8 (MR-32172) and a bicistronic expression repressor (MR-32905) targeting the E1 cRE and IL8.

FIG. 52 are bar graphs showing the CXCL gene and protein downregulation in bronchial smooth muscle cells (asthma) after introduction of an expression repressor targeting IL8 (MR-32172) and a bicistronic expression repressor (MR-32905) targeting the E1 cRE and IL8.

FIG. 53 are bar graphs showing the CXCL gene and protein downregulation in primary lung fibroblast cells after introduction of an expression repressor targeting IL8 (MR-32172) and a bicistronic expression repressor (MR-32905) targeting the E1 cRE and IL8.

FIG. 54 are graphs showing the CXCL1-3 and IL8 downregulation over 13 days after introduction of an expression repressor targeting IL8 (MR-32172) and a bicistronic expression repressor (MR-32905) targeting the E1 cRE and IL8.

FIG. 55 are graphs showing decreased neutrophil migration after introduction of an expression repressor targeting the E1 cRE (MR-32105) and/or an expression repressor targeting IL8 (MR-32712).

FIGS. 56A and 56B are graphs showing decreased neutrophil migration after introduction of an expression repressor targeting IL8 (MR-32712) and/or a bicistronic expression repressor (MR-32905) targeting the E1 cRE and IL8.

FIG. 57 is an image depicting the locus of the functional enhancers at the CXCL cluster in mouse. Three candidate E1 locations tested in Example 41 are indicated with arrows.

FIG. 58 are bar graphs indicating CXCL1 and CXCL2 downregulation after instruction of an expression repressor with a guide targeting mouse P1 and P6, homologues to human E1 and E2, respectively.

FIG. 59 is a bar graph indicating CXCL2 RNA qPCR results after instruction of an expression repressor with a guide targeting mouse homologues to human CXCL.

FIG. 60 is a bar graph indicating CXCL1 RNA qPCR results after instruction of an expression repressor with a guide targeting mouse homologues to human CXCL.

FIG. 61 is a bar graph indicating CXCL1 protein expression results after introduction of an expression repressor with a guide targeting mouse homologues to human CXCL.

FIG. 62 are bar graphs indicating CXCL1 and CXCL2 downregulation in mouse homologues to human CXCL.

FIG. 63 is a bar graph indicating CXCL1 protein expression results after introduction of expression repressors targeting a mouse homologue to human CXCL1.

FIG. 64 is a bar graph indicating IL-8 mRNA level results after introduction of bicistronic expression repressor (MR-32905) targeting the E1 cRE and IL8 in multiple cancer cell lines. IL-8 mRNA levels are normalized to IL-8 mRNA in TNFα-stimulated cells.

FIG. 65 is a bar graph indicating IL-8 protein expression level results after introduction of bicistronic expression repressor (MR-32905) targeting the E1 cRE and IL8 in multiple cancer cell lines. IL-8 mRNA levels are normalized to IL-8 mRNA in TNFα-stimulated cells.

FIG. 66 is a bar graph indicating CXCL1 mRNA level results after introduction of bicistronic expression repressor (MR-32905) targeting the E1 cRE and IL8 in multiple cancer cell lines. CXCL1 mRNA levels are normalized to CXCL1 mRNA in TNFα-stimulated cells.

FIG. 67 is a bar graph indicating endogenous IL-8 mRNA level results after introduction of bicistronic expression repressor (MR-32905) targeting the E1 cRE and IL8 in a breast cancer cell line. IL-8 mRNA levels are normalized to IL-8 mRNA in TNFα-stimulated cells.

FIG. 68 is a graph indicating tumor volume (mm³) in A549 NSCLC xenograft model after introduction of bicistronic expression repressor (MR-32905) targeting the E1 cRE and IL8.

FIG. 69 is a graph indicating the mean percent weight change in A549 NSCLC xenograft model mouse groups. Error bars represent the standard error of the mean (SEM). This experiment was performed as described in Example 47.

FIG. 70 is a bar graph indicating the percent weight change Area Under the Curve (AUC) in A549 NSCLC xenograft model mouse groups. The percent weight change AUC was calculated for each animal in the study to Day 04. This calculation was made using the trapezoidal rule transformation. Error bars represent the SEM for each group. This experiment was performed as described in Example 47.

FIG. 71 is a graph depicting the mean tumor volumes (mm³) in A549 NSCLC xenograft model after introduction of bicistronic expression repressor (MR-32905) or GFP control. Mean tumor volumes were calculated from the length and width measurements. Group means were calculated and are shown with error bars representing SEM for each group. This experiment was performed as described in Example 47.

FIG. 72 is a bar graph indicating the percent weight change Area Under the Curve (AUC) in A549 NSCLC xenograft model mouse groups. The AUC was calculated using the trapezoidal rule transformation for the tumor volume measured on each animal in the study. Group means were calculated and are shown with error bars representing SEM for each group. Groups were compared using an ANOVA test. This experiment was performed as described in Example 47.

FIG. 73 is a graph indicating the mean percent tumor volumes in A549 NSCLC xenograph model after introduction of bicistronic expression repressor (MR-32905) or GFP control. Mean Tumor Volumes were calculated from the length and width measurements. Group means were calculated and are shown with error bars representing SEM for each group. This experiment was performed as described in Example 47.

FIG. 74 is bar graph indicating the percent weight change Area Under the Curve (AUC) in A549 NSCLC xenograft model mouse groups. The AUC was calculated for the tumor volume measured on each animal in the study. This calculation was made using the trapezoidal rule transformation. Group means were calculated and are shown with error bars representing SEM for each group. Groups were compared using an ANOVA test. This experiment was performed as described in Example 47.

FIG. 75 is a schematic experimental design to determine the effects of expression repressors for use in acute respiratory distress syndrome (ARDS). This experiment was performed as described in Example 48.

FIG. 76 is a graph showing change in body weight (BW) percent from baseline in LPS induced C57BL/6 mice. This experiment was performed as described in Example 48.

FIG. 77 is a bar graph showing BALF cell concentration in C57BL/6 mice. This experiment was performed as described in Example 48.

FIGS. 78A-78E are bar graphs showing BALF immune cell concentrations in LPS induced C57BL/6 mice. FIG. 78A is a bar graph showing BALF mouse leukocyte concentration (Cells/mL). FIG. 78B is a bar graph showing BALF mouse alveolar macrophage concentration (Cells/mL). FIG. 78C is a bar graph showing BALF mouse neutrophil concentration (Cells/mL). FIG. 78D is a bar graph showing BALF mouse T cell concentration (Cells/mL). FIG. 78E is a bar graph showing BALF mouse B cell concentration (Cells/mL). This experiment was performed as described in Example 48.

FIGS. 79A-79D are bar graphs indicating BALF immune cell frequency in LPS induced C57BL/6 mice. This experiment was performed as described in Example 48.

FIGS. 80A-80E are bar graphs indicating blood immune cell concentrations in LPS induced C57BL/6 mice. This experiment was performed as described in Example 48.

FIGS. 81A-81D are bar graphs indicating blood immune cell frequency in LPS induced C57BL/6 mice. This experiment was performed as described in Example 48.

FIGS. 82A-82F are bar graphs indicating the histology score and assessment in LPS induced C57BL/6 mice. This experiment was performed as described in Example 48.

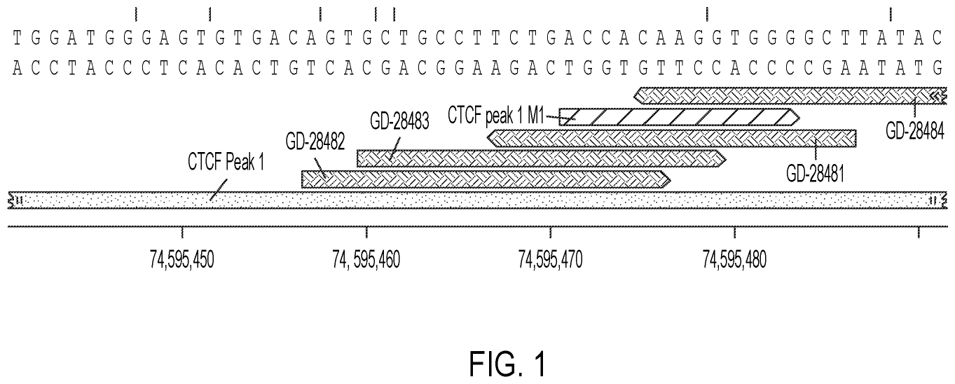

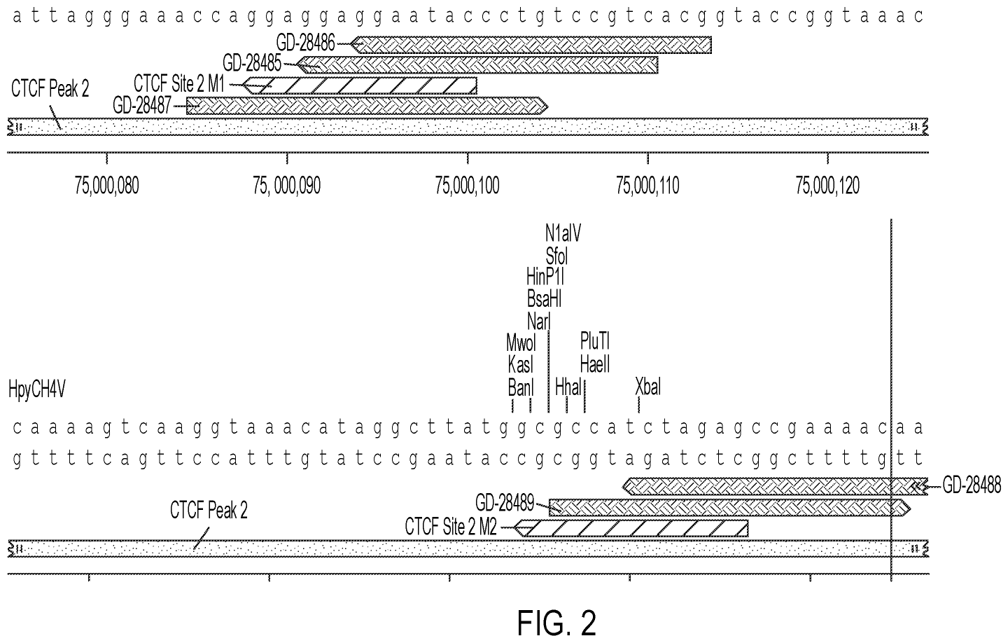

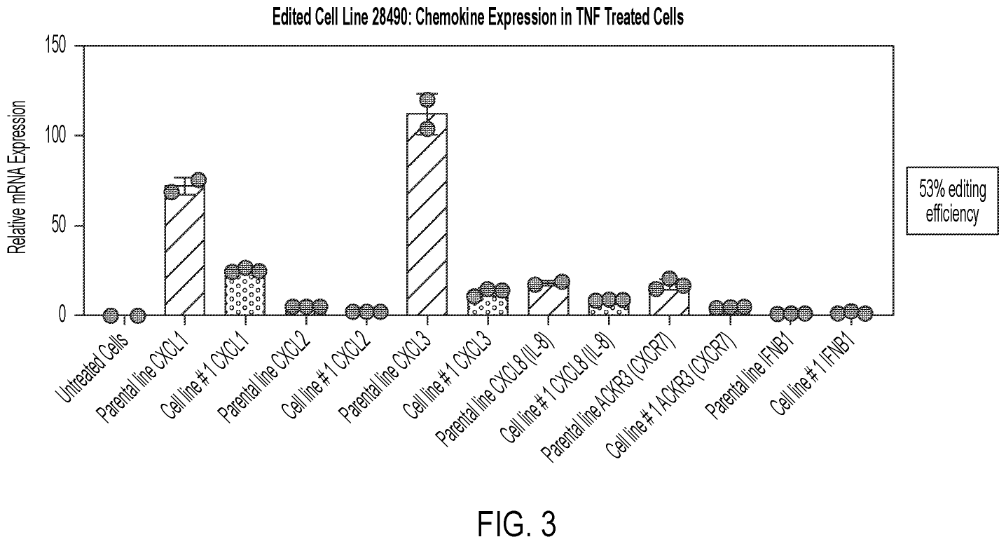

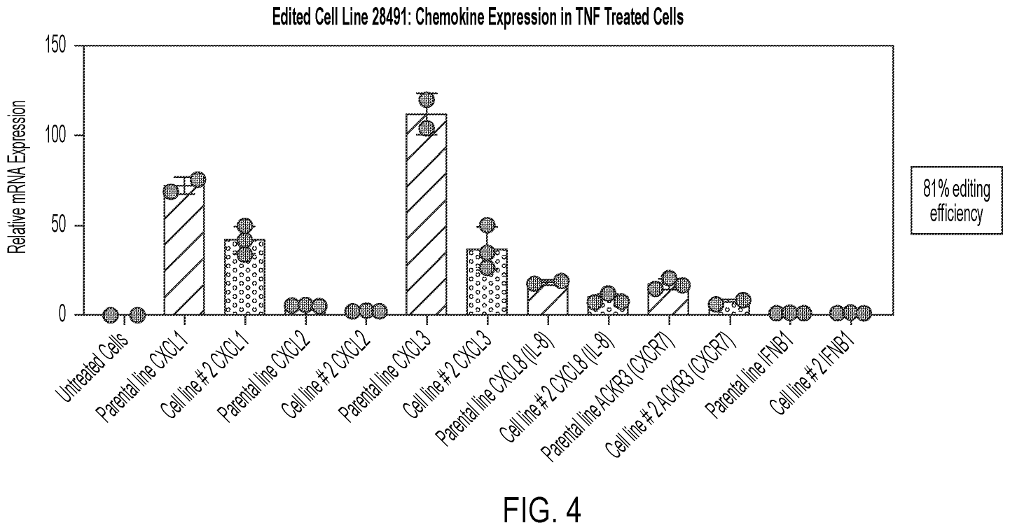

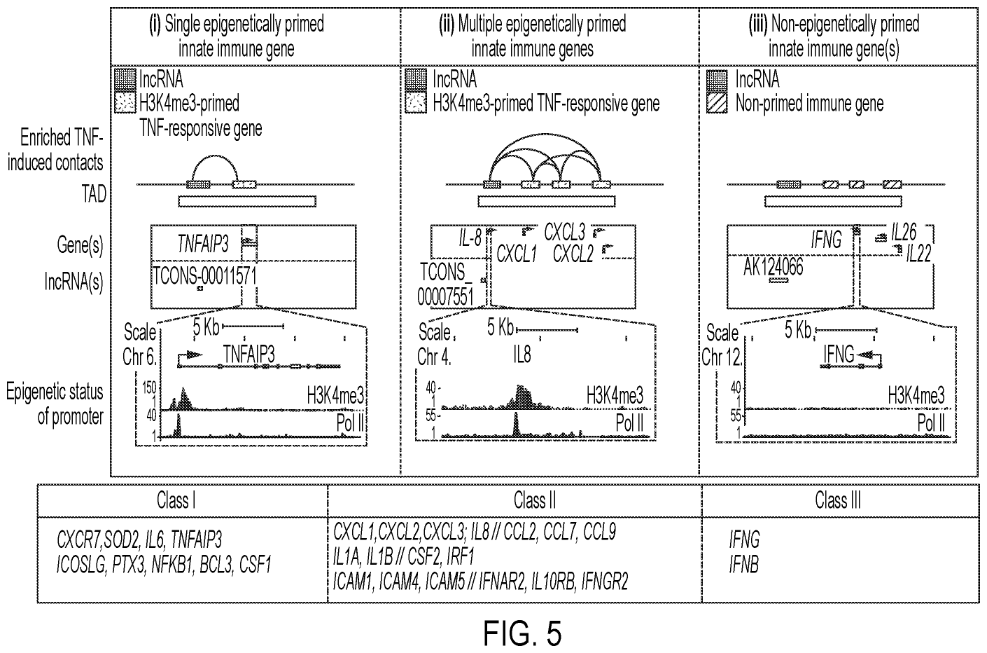

### DETAILED DESCRIPTION OF CERTAIN EMBODIMENTS

The present disclosure provides, e.g., technologies for decreasing expression of a target plurality of CXCL genes in a cell, e.g., in a subject or patient, through the use of an expression repressor, a system comprising two or more expression repressors, or a system comprising an expression repressor and a site-specific disrupting agent. In some embodiments, an expression repressor comprises a targeting moiety. In some embodiments, an expression repressor comprises a targeting moiety and an effector moiety. Without wishing to be bound by theory, a number of diseases and conditions are associated with groups of genes with related functionalities that are associated with a common enhancer. Inhibition of the enhancer may be an improved approach to decreasing expression of the target plurality of genes (e.g., with respect to improved efficiency, efficacy, and/or stability of alteration) over modulation of individual target genes. Optionally, the expression repressor may be used in combination with a site-specific disrupting agent, e.g., a site-specific disrupting agent that disrupts an anchor sequence mediated conjunction. The site-specific disrupting agent may also repress expression of a plurality of genes (e.g., the same plurality of genes as the expression repressor or an overlapping plurality of genes). Said improvements may translate to corresponding improvements in the treatment of diseases and conditions associated with the target plurality of genes. For example, a plurality of genes may be CXCL genes and an expression repressor can target an E1 cRE, operably linked to the plurality of genes to decrease expression of the plurality of genes and thereby achieve an anti-inflammatory effect (e.g., a superior anti-inflammatory effect relative to individually targeting the genes of the plurality). Examples of expression repressor, site-specific disrupting agents, targeting moieties, effector moieties, and target pluralities of genes are provided herein.

An expression repressor may decrease expression of a target plurality of genes by one or more modalities. In some embodiments, an expression repressor to a target site, e.g., an E1 cRE, may physically or sterically compete for binding with a factor that binds the target site. Without wishing to be bound by theory, physical or steric blockage of an enhancer sequence (e.g., an E1 cRE), e.g., such that binding of a factor to the enhancer sequence is inhibited (e.g., prevented), is one mechanism by which an expression repressor may modulate, e.g., decrease, expression of a target plurality of genes. An expression repressor may destabilize the interaction of a factor) with an enhancer sequence, e.g., by altering (e.g., decreasing) the affinity and/or avidity at which the factor binds the enhancer sequence. Blocking or destabilizing binding of a factor to a target sequence may be accomplished by one or more means, including: epigenetic modification of the enhancer sequence or a sequence proximal thereto, genetic modification of the enhancer sequence or a sequence proximal thereto, or binding of the expression repressor to the enhancer sequence or a sequence proximal thereto. Inhibition of a genomic regulatory element operably linked to a target plurality of genes may modulate, e.g., decrease, expression of the genes of the target plurality of genes. In some embodiments, an expression repressor comprises a targeting moiety, a first effector moiety, and a second effector moiety. In some embodiments, the first effector moiety has a sequence that is different from the sequence of the second effector moiety. In some embodiments, the first effector moiety has a sequence that is identical to the sequence of the second effector moiety.

An expression repressor described herein (e.g., one that targets an enhancer sequence) may also be used in combination with a site-specific disrupting agent (e.g., one that targets an anchor sequence.) In some embodiments, a site-specific disrupting agent comprises a targeting moiety. In some embodiments, a site-specific disrupting agent comprises a targeting moiety and an effector moiety. Without wishing to be bound by theory, a number of diseases and conditions are associated with groups of genes with related functionalities that are associated with a common genomic complex, e.g., ASMC. Modulation, e.g., disruption, of a genomic complex, e.g., ASMC, comprising (wholly or in part) a target plurality of genes may be an improved approach to altering (e.g., decreasing) expression of the target plurality of genes (e.g., with respect to improved efficiency, efficacy, and/or stability of alteration) over modulation of individual target genes. Said improvements may translate to corresponding improvements in the treatment of diseases and conditions associated with the target plurality of genes. For example, a plurality of genes may be associated with a pro-inflammatory effect and a site-specific disrupting agent can target a genomic complex, e.g., ASMC, comprising (wholly or in part) the plurality of genes to modulate, e.g., decrease, expression of the plurality of genes and thereby achieve an anti-inflammatory effect (e.g., a superior anti-inflammatory effect relative to individually targeting the genes of the plurality). Examples of site-specific disrupting agents, targeting moieties, effector moieties, and target pluralities of genes are provided herein.

A site-specific disrupting agent may modulate, e.g., decrease, expression of a target plurality of genes by one or more modalities. In some embodiments, a site-specific disrupting agent binds to a target site, e.g., anchor sequence, and physically or sterically competes for binding with other genomic complex components, e.g., a nucleating polypeptide. Without wishing to be bound by theory, physical or steric blockage of an anchor sequence, e.g., such that binding of a genomic complex component (e.g., a nucleating polypeptide) to the anchor sequence is inhibited (e.g., prevented), is one mechanism by which a site-specific disrupting agent may modulate, e.g., decrease, expression of a target plurality of genes. A site-specific disrupting agent may destabilize the interaction of a genomic complex component (e.g., nucleating polypeptide) with an anchor sequence, e.g., by altering (e.g., decreasing) the affinity and/or avidity at which the genomic complex component binds the anchor sequence. Blocking or destabilizing binding of a genomic complex component (e.g., nucleating polypeptide) to an anchor sequence may be accomplished by one or more means, including: epigenetic modification of the anchor sequence or a sequence proximal thereto, genetic modification of the anchor sequence or a sequence proximal thereto, or binding of the site-specific disrupting agent to the anchor sequence or a sequence proximal thereto. Inhibiting (e.g., preventing) binding of a genomic complex component (e.g., a nucleating polypeptide) to an anchor sequence may inhibit (e.g., disrupt or prevent formation of) a genomic complex, e.g., ASMC. Inhibition of a genomic complex, e.g., ASMC, comprising, wholly or partly, a target plurality of genes may modulate, e.g., decrease, expression of the genes of the target plurality of genes. In some embodiments, a site-specific disrupting agent comprises a targeting moiety, a first effector moiety, and a second effector moiety. In some embodiments, the first effector moiety has a sequence that is different from the sequence of the second effector moiety. In some embodiments, the first effector moiety has a sequence that is identical to the sequence of the second effector moiety.

The disclosure further provides in part, a system comprising two or more expression repressors, each comprising a targeting moiety and optionally an effector moiety. In some embodiments, the targeting moieties target two or more different sequences (e.g., each expression repressor may target a different sequence). In some embodiments, the first expression repressor binds to a first genomic regulatory element (e.g., an enhancer, e.g., an E1 cRE) operably linked to a target plurality of genes, e.g., human CXCL1-8, and the second expression repressor binds to a second genomic regulatory element (e.g., an enhancer, a promoter, or a transcription start site TSS)) operably linked to the plurality of genes e.g., human CXCL1-8. In some embodiments, the system comprises an expression repressor and a site-specific disrupting agent. In some embodiments, the expression repressor binds to a transcription regulatory element (e.g., an enhancer (e.g., an E1 cRE) operably linked to a target plurality of genes, e.g., human CXCL1-8 and the site-specific disrupting agent binds to an anchor sequence of an anchor sequence mediated conjunction (ASMC) comprising a target plurality of genes, e.g., human CXCL1-8.

In some embodiments, modulation of expression of a target plurality of genes, e.g., human CXCL 1-8 by a system involves the binding of the first expression repressor and second expression repressor to the first and second DNA sequences, respectively. In some embodiments, modulation of expression of a target plurality of genes, e.g., human CXCL1-8 by a system involves the binding of the expression repressor and the site-specific disrupting agent to the first and second DNA sequences, respectively.

Binding of the first and second DNA sequences localizes the functionalities of the first and second effector moieties to those sites. Without wishing to be bound by theory, in some embodiments employing the functionalities of both the first and second effector moieties stably represses expression of a target plurality of gene associated with or comprising the first and/or second DNA sequences, e.g., wherein the first and/or second DNA sequences are or comprise sequences of the target plurality of gene or one or more operably linked genomic regulatory elements (e.g., transcription control elements).

### Expression Repressors

In some embodiments, an expression repressor comprises a targeting moiety. In some embodiments, the targeting moiety specifically binds a DNA sequence, e.g., an E1 cRE, and thereby modulates, e.g., disrupts, the function of that DNA sequence. In some embodiments, an expression repressor comprises a targeting moiety and an effector moiety. In some embodiments, the targeting moiety specifically binds a DNA sequence, thereby localizing the effector moiety's functionality to the DNA sequence or an area proximal thereto. In some embodiments, an expression repressor comprises one targeting moiety and one effector moiety. In some embodiments, an expression repressor comprises one targeting moiety and more than one effector moiety, e.g., two, three, four, or five effector moieties, each of which may be the same or different from another of the more than one effector moieties. In some embodiments, an expression repressor may comprise two effector moieties where the first effector moiety comprises a different functionality than the second effector moiety. For example, an expression repressor may comprise two effector moieties, where the first effector moiety comprises DNA methyltransferase functionality (e.g., comprises MQ1, G9A, or EZH2, or a functional fragment or variant thereof) and the second effector moiety comprises a transcriptional repressor functionality (e.g., comprises KRAB or a functional fragment or variant thereof). In some embodiments, an expression repressor comprises effector moieties whose functionalities are complementary to one another with regard to decreasing expression of a target plurality of gene, where the functionalities together inhibit expression and, optionally, do not inhibit or negligibly inhibit expression when present individually. In some embodiments, an expression repressor comprises a plurality of effector moieties, wherein each effector moiety complements each other effector moiety, each effector moiety decreases expression of a target plurality of gene.

In some embodiments, an expression repressor comprises a combination of effector moieties whose functionalities synergize with one another with regard to decreasing expression of a target plurality of gene. Without wishing to be bound by theory, in some embodiments, epigenetic modifications to a genomic locus are cumulative, in that multiple transcription activating epigenetic markers (e.g., multiple different types of epigenetic markers and/or more extensive marking of a given type) individually together inhibit expression more effectively than individual modifications alone (e.g., producing a greater decrease in expression and/or a longer-lasting decrease in expression). In some embodiments, an expression repressor comprises a plurality of effector moieties, wherein each effector moiety synergizes with each other effector moiety, e.g., each effector moiety decreases expression of a target plurality of gene. In some embodiments, an expression repressor (comprising a plurality of effector moieties which synergize with one another) is more effective at inhibiting expression of a target plurality of gene, than an expression repressor comprising an individual effector moiety. In some embodiments, an expression repressor comprising said plurality of effector moieties is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× as effective at decreasing expression of a target plurality of gene, than an expression repressor comprising an individual effector moiety.

In some embodiments, an expression repressor comprises one or more targeting moieties, e.g., a Cas domain, TAL effector domain, or Zn Finger domain. In an embodiment, when a system comprises two or more targeting moieties of the same type, e.g., two or more Cas domains or two or more Zn Finger Domains, the targeting moieties specifically bind two or more different sequences. As a non-limiting example, an expression repressor system comprising two or more Zinc Finger domains, the two or more Zinc Finger domains may be chosen or altered such that they only appreciably bind their target sequence (e.g., and do not appreciably bind the target of another Zinc Finger domain). As another non-limiting example, in an expression repressor system comprising two or more Cas domains, the two or more Cas domains may be chosen or altered such that they only appreciably bind the gRNA corresponding to their target sequence (e.g., and do not appreciably bind the gRNA corresponding to the target of another Cas domain).

In some embodiments, an expression repressor comprises a targeting moiety and an effector moiety that are covalently linked, e.g., by a peptide bond. In some embodiments, the targeting moiety and effector moiety are situated on the same polypeptide chain, e.g., connected by one or more peptide bonds and/or a linker. In some embodiments an expression repressor comprises a fusion molecule, e.g., comprising the targeting moiety and effector moiety linked by a peptide bond and/or a linker. In some embodiments, an expression repressor comprises a targeting moiety that is disposed N-terminal of an effector moiety on the same polypeptide chain. In some embodiments, an expression repressor comprises a targeting moiety that is disposed C-terminal of an effector moiety on the same polypeptide chain. In some embodiments, an expression repressor comprises a targeting moiety and an effector moiety that are covalently linked by a non-peptide bond. In some embodiments, a targeting moiety is conjugated to an effector moiety by a non-peptide bond. In some embodiments, an expression repressor comprises a targeting moiety and a plurality of effector moieties, wherein the targeting moiety and the plurality of effector moieties are covalently linked, e.g., by peptide bonds (e.g., the targeting moiety and plurality of effector moieties are all connected by a series of covalent bonds, although each individual moiety may not share a covalent bond with every other moiety).

In other embodiments, an expression repressor comprises a targeting moiety and an effector moiety that are not covalently linked, e.g., that are non-covalently associated with one another. In some embodiments, an expression repressor comprises a targeting moiety that non-covalently binds to an effector moiety or vice versa. In some embodiments, an expression repressor comprises a targeting moiety and a plurality of effector moieties, wherein the targeting moiety and at least one effector moiety are not covalently linked, e.g., are non-covalently associated with one another, and wherein the targeting moiety and at least one other effector moiety are covalently linked, e.g., by a peptide bond.

In some embodiments, an expression repressor comprises a first effector moiety comprising G9A and a second effector moiety comprising KRAB. In some embodiments an expression repressor comprises a first effector moiety comprising G9A and a second effector moiety comprising EZH2. In some embodiments, an expression repressor comprises a first effector moiety comprising EZH2 and a second effector moiety comprising KRAB.

In some embodiments, an expression repressor comprises a targeting moiety and an effector moiety, wherein the C-terminal end of the effector moiety, e.g., an effector moiety chosen from, KRAB or MQ1 or a functional variant or fragment thereof and the N-terminal end of the targeting moiety are covalently linked. In some embodiments, an expression repressor comprises a targeting moiety and an effector moiety wherein the N-terminal end of the effector moiety, e.g., an effector moiety chosen from HDAC8, MQ1, DNMT3a/3L, KRAB, or a functional variant or fragment thereof and the C-terminal end of the targeting moiety are covalently linked. In some embodiments, an expression repressor comprises a targeting moiety, a first effector moiety and a second effector moiety, wherein, the C-terminal end of the first effector moiety and the N-terminal end of the targeting moiety are covalently linked and the C-terminal end of the targeting moiety and the N-terminal end of the second effector moiety are covalently linked. The covalent linkage may be, e.g., via a linker sequence.

In some embodiments, an expression repressor comprises a targeting moiety, a first effector moiety and a second effector moiety, wherein the first effector moiety is EZH2, or a functional variant or fragment thereof, e.g., wherein the first effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is KRAB, or a functional variant or fragment thereof, e.g., wherein the second effector moiety comprises an amino acid sequence of SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, a site-specific disrupting agent comprises a targeting moiety, a first effector moiety and a second effector moiety, wherein the first effector moiety is EZH2, or a functional variant or fragment thereof, e.g., wherein the first effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is HDAC8, or a functional variant or fragment thereof, e.g., wherein the second effector moiety comprises an amino acid sequence of SEQ ID NO: 19 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety. In some embodiments, a site-specific disrupting agent comprises a targeting moiety, a first effector moiety and a second effector moiety, wherein the first effector moiety is G9A, or a functional variant or fragment thereof, e.g., wherein the first effector moiety comprises an amino acid sequence of SEQ ID NO: 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is KRAB, or a functional variant or fragment thereof, e.g., wherein the second effector moiety comprises an amino acid sequence of SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, a site-specific disrupting agent comprises a targeting moiety, a first effector moiety and a second effector moiety, wherein the first effector moiety is G9A, or a functional variant or fragment thereof, e.g., wherein the first effector moiety comprises an amino acid sequence of SEQ ID NO: 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is EZH2, or a functional variant or fragment thereof, e.g., wherein the second effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, the first effector moiety comprises a histone methyltransferase activity and the second effector moiety comprises a different histone methyltransferase activity. In some embodiments, the first effector moiety comprises a histone methyltransferase activity and the second effector moiety comprises the same histone methyltransferase activity. In some embodiments, the first effector moiety comprises a histone demethylase activity and the second effector moiety comprises a histone deacetylase activity. In some embodiments, the first effector moiety comprises a histone demethylase activity and the second effector moiety comprises a DNA methyltransferase activity. In some embodiments, the first effector moiety comprises a histone demethylase activity and the second effector moiety comprises a DNA demethylase activity. In some embodiments, the first effector moiety comprises a histone demethylase activity and the second effector moiety comprises a transcription repressor activity. In some embodiments, the first effector moiety comprises a histone demethylase activity and the second effector moiety comprises a different histone demethylase activity. In some embodiments, the first effector moiety comprises a histone demethylase activity and the second effector moiety comprises the same histone demethylase activity. In some embodiments, the first effector moiety comprises a histone deacetylase activity and the second effector moiety comprises a DNA methyltransferase activity. In some embodiments, the first effector moiety comprises a histone deacetylase activity and the second effector moiety comprises a DNA demethylase activity. In some embodiments, the first effector moiety comprises a histone deacetylase activity and the second effector moiety comprises a transcription repressor activity. In some embodiments, the first effector moiety comprises a histone deacetylase activity and the second effector moiety comprises a different histone deacetylase activity. In some embodiments, the first effector moiety comprises a histone deacetylase activity and the second effector moiety comprises the same histone deacetylase activity. In some embodiments, the first effector moiety comprises a DNA methyltransferase activity and the second effector moiety comprises a DNA demethylase activity. In some embodiments, the first effector moiety comprises a DNA methyltransferase activity and the second effector moiety comprises a transcription repressor activity. In some embodiments, the first effector moiety comprises a DNA methyltransferase activity and the second effector moiety comprises a different DNA methyltransferase activity. In some embodiments, the first effector moiety comprises a DNA methyltransferase activity and the second effector moiety comprises the same DNA methyltransferase activity. In some embodiments, the first effector moiety comprises a DNA demethylase activity and the second effector moiety comprises a transcription repressor activity. In some embodiments, the first effector moiety comprises a DNA demethylase activity and the second effector moiety comprises a different DNA demethylase activity. In some embodiments, the first effector moiety comprises a DNA demethylase activity and the second effector moiety comprises the same DNA demethylase activity. In some embodiments, the first effector moiety comprises a transcription repressor activity and the second effector moiety comprises a different transcription repressor activity. In some embodiments, the first effector moiety comprises a transcription repressor activity and the second effector moiety comprises the same transcription repressor activity.

In some embodiments, the first effector moiety comprises DNMT3a/3l, MQ1, KRAB, G9A, HDAC8, or EZH2 and the second effector moiety comprises DNMT3a/3l, MQ1, KRAB, G9A, HDAC8, or EZH2.

### Site-Specific Disrupting Agents

In some embodiments, a site-specific disrupting agent comprises a targeting moiety. In some embodiments, the targeting moiety specifically binds a DNA sequence, e.g., an anchor sequence, and thereby modulates, e.g., disrupts, a genomic complex (e.g., ASMC) comprising said DNA sequence. In some embodiments, a site-specific disrupting agent comprises a targeting moiety and an effector moiety. In some embodiments, the targeting moiety specifically binds a DNA sequence, thereby localizing the effector moiety's functionality to the DNA sequence, thereby modulating, e.g., disrupting, a genomic complex (e.g., ASMC) comprising said DNA sequence. In some embodiments, a site-specific disrupting agent comprises one targeting moiety and one effector moiety. In some embodiments, a site-specific disrupting agent comprises one targeting moiety and more than one effector moiety, e.g., two, three, four, or five effector moieties, each of which may be the same or different from another of the more than one effector moieties. In some embodiments, a site-specific disrupting agent may comprise two effector moieties where the first effector moiety comprises a different functionality than the second effector moiety. For example, a site-specific disrupting agent may comprise two effector moieties, where the first effector moiety comprises DNA methyltransferase functionality (e.g., comprises G9A or EZH2 or a functional fragment or variant thereof) and the second effector moiety comprises a transcriptional repressor functionality (e.g., comprises KRAB or a functional fragment or variant thereof). In some embodiments, a site-specific disrupting agent comprises effector moieties whose functionalities are complementary to one another with regard to decreasing expression of a target plurality of gene, where the functionalities together inhibit expression and, optionally, do not inhibit or negligibly inhibit expression when present individually. In some embodiments, a site-specific disrupting agent comprises a plurality of effector moieties, wherein each effector moiety complements each other effector moiety, each effector moiety decreases expression of a target plurality of gene.

In some embodiments, a site-specific disrupting agent comprises a combination of effector moieties whose functionalities synergize with one another with regard to decreasing expression of a target plurality of gene. Without wishing to be bound by theory, in some embodiments, epigenetic modifications to a genomic locus are cumulative, in that multiple transcription activating epigenetic markers (e.g., multiple different types of epigenetic markers and/or more extensive marking of a given type) individually together inhibit expression more effectively than individual modifications alone (e.g., producing a greater decrease in expression and/or a longer-lasting decrease in expression). In some embodiments, a site-specific disrupting agent comprises a plurality of effector moieties, wherein each effector moiety synergizes with each other effector moiety, e.g., each effector moiety decreases expression of a target plurality of gene. In some embodiments, a site-specific disrupting agent (comprising a plurality of effector moieties which synergize with one another) is more effective at inhibiting expression of a target plurality of gene, than a site-specific disrupting agent comprising an individual effector moiety. In some embodiments, a site-specific disrupting agent comprising said plurality of effector moieties is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× as effective at decreasing expression of a target plurality of gene, than a site-specific disrupting agent comprising an individual effector moiety.

In some embodiments, a site-specific disrupting agent comprises one or more targeting moieties e.g., a Cas domain, TAL effector domain, or Zn Finger domain. In an embodiment, when system comprises two or more targeting moieties of the same type, e.g., two or more Cas domains, the targeting moieties specifically bind two or more different sequences. For example, in a site-specific disrupting agent system comprising two or more Cas domains, the two or more Cas domains may be chosen or altered such that they only appreciably bind the gRNA corresponding to their target sequence (e.g., and do not appreciably bind the gRNA corresponding to the target of another Cas domain).

In some embodiments, a site-specific disrupting agent comprises a targeting moiety and an effector moiety that are covalently linked, e.g., by a peptide bond. In some embodiments, the targeting moiety and effector moiety are situated on the same polypeptide chain, e.g., connected by one or more peptide bonds and/or a linker. In some embodiments, a site-specific disrupting agent comprises a fusion molecule, e.g., comprising the targeting moiety and effector moiety linked by a peptide bond and/or a linker. In some embodiments, a site-specific disrupting agent comprises a targeting moiety that is disposed N-terminal of an effector moiety on the same polypeptide chain. In some embodiments, a site-specific disrupting agent comprises a targeting moiety that is disposed C-terminal of an effector moiety on the same polypeptide chain. In some embodiments, a site-specific disrupting agent comprises a targeting moiety and an effector moiety that are covalently linked by a non-peptide bond. In some embodiments, a targeting moiety is conjugated to an effector moiety by a non-peptide bond. In some embodiments, a site-specific disrupting agent comprises a targeting moiety and a plurality of effector moieties, wherein the targeting moiety and the plurality of effector moieties are covalently linked, e.g., by peptide bonds (e.g., the targeting moiety and plurality of effector moieties are all connected by a series of covalent bonds, although each individual moiety may not share a covalent bond with every other moiety).

In other embodiments, a site-specific disrupting agent comprises a targeting moiety and an effector moiety that are not covalently linked, e.g., that are non-covalently associated with one another. In some embodiments, a site-specific disrupting agent comprises a targeting moiety that non-covalently binds to an effector moiety or vice versa. In some embodiments, a site-specific disrupting agent comprises a targeting moiety and a plurality of effector moieties, wherein the targeting moiety and at least one effector moiety are not covalently linked, e.g., are non-covalently associated with one another, and wherein the targeting moiety and at least one other effector moiety are covalently linked, e.g., by a peptide bond.

In some embodiments, a site-specific disrupting agent comprises a first effector moiety comprising G9A and a second effector moiety comprising KRAB. In some embodiments, a site-specific disrupting agent comprises a first effector moiety comprising G9A and a second effector moiety comprising EZH2. In some embodiments, a site-specific disrupting agent comprises a first effector moiety comprising EZH2 and a second effector moiety comprising KRAB.

In some embodiments, a site-specific disrupting agent comprises a targeting moiety and an effector moiety, wherein the C-terminal end of the effector moiety, e.g., an effector moiety chosen from, EZH2, or G9A or a functional variant or fragment thereof and the N-terminal end of the targeting moiety are covalently linked. In some embodiments, a site-specific disrupting agent comprises a targeting moiety and an effector moiety wherein the N-terminal end of the effector moiety, e.g., an effector moiety chosen from HDAC8, MQ1, DNMT3a/3L, KRAB, or a functional variant or fragment thereof and the C-terminal end of the targeting moiety are covalently linked. In some embodiments, a site-specific disrupting agent comprises a targeting moiety, a first effector moiety and a second effector moiety, wherein, the C-terminal end of the first effector moiety, e.g., a effector moiety chosen from EZH2, G9A, or a functional variant or fragment thereof, and the N-terminal end of the targeting moiety are covalently linked and the C-terminal end of the targeting moiety and the N-terminal end of the second effector moiety, e.g., a effector moiety chosen from HDAC8, MQ1, DNMT3a/3L, KRAB or a functional variant or fragment thereof are covalently linked. The covalent linkage may be, e.g., via a linker sequence.

In some embodiments, a site-specific disrupting agent comprises a targeting moiety, a first effector moiety and a second effector moiety, wherein the first effector moiety is EZH2, or a functional variant or fragment thereof, e.g., wherein the first effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is KRAB, or a functional variant or fragment thereof, e.g., wherein the second effector moiety comprises an amino acid sequence of SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, a site-specific disrupting agent comprises a targeting moiety, a first effector moiety and a second effector moiety, wherein the first effector moiety is EZH2, or a functional variant or fragment thereof, e.g., wherein the first effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is HDAC8, or a functional variant or fragment thereof, e.g., wherein the second effector moiety comprises an amino acid sequence of SEQ ID NO: 19 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, a site-specific disrupting agent comprises a targeting moiety, a first effector moiety and a second effector moiety, wherein the first effector moiety is G9A, or a functional variant or fragment thereof, e.g., wherein the first effector moiety comprises an amino acid sequence of SEQ ID NO: 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is KRAB, or a functional variant or fragment thereof, e.g., wherein the second effector moiety comprises an amino acid sequence of SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, a site-specific disrupting agent comprises a targeting moiety, a first effector moiety and a second effector moiety, wherein the first effector moiety is G9A, or a functional variant or fragment thereof, e.g., wherein the first effector moiety comprises an amino acid sequence of SEQ ID NO: 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is EZH2, or a functional variant or fragment thereof, e.g., wherein the second effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, the first effector moiety comprises a histone methyltransferase activity and the second effector moiety comprises a different histone methyltransferase activity. In some embodiments, the first effector moiety comprises a histone methyltransferase activity and the second effector moiety comprises the same histone methyltransferase activity. In some embodiments, the first effector moiety comprises a histone demethylase activity and the second effector moiety comprises a histone deacetylase activity. In some embodiments, the first effector moiety comprises a histone demethylase activity and the second effector moiety comprises a DNA methyltransferase activity. In some embodiments, the first effector moiety comprises a histone demethylase activity and the second effector moiety comprises a DNA demethylase activity. In some embodiments, the first effector moiety comprises a histone demethylase activity and the second effector moiety comprises a transcription repressor activity. In some embodiments, the first effector moiety comprises a histone demethylase activity and the second effector moiety comprises a different histone demethylase activity. In some embodiments, the first effector moiety comprises a histone demethylase activity and the second effector moiety comprises the same histone demethylase activity. In some embodiments, the first effector moiety comprises a histone deacetylase activity and the second effector moiety comprises a DNA methyltransferase activity. In some embodiments, the first effector moiety comprises a histone deacetylase activity and the second effector moiety comprises a DNA demethylase activity. In some embodiments, the first effector moiety comprises a histone deacetylase activity and the second effector moiety comprises a transcription repressor activity. In some embodiments, the first effector moiety comprises a histone deacetylase activity and the second effector moiety comprises a different histone deacetylase activity. In some embodiments, the first effector moiety comprises a histone deacetylase activity and the second effector moiety comprises the same histone deacetylase activity. In some embodiments, the first effector moiety comprises a DNA methyltransferase activity and the second effector moiety comprises a DNA demethylase activity. In some embodiments, the first effector moiety comprises a DNA methyltransferase activity and the second effector moiety comprises a transcription repressor activity. In some embodiments, the first effector moiety comprises a DNA methyltransferase activity and the second effector moiety comprises a different DNA methyltransferase activity. In some embodiments, the first effector moiety comprises a DNA methyltransferase activity and the second effector moiety comprises the same DNA methyltransferase activity. In some embodiments, the first effector moiety comprises a DNA demethylase activity and the second effector moiety comprises a transcription repressor activity. In some embodiments, the first effector moiety comprises a DNA demethylase activity and the second effector moiety comprises a different DNA demethylase activity. In some embodiments, the first effector moiety comprises a DNA demethylase activity and the second effector moiety comprises the same DNA demethylase activity. In some embodiments, the first effector moiety comprises a transcription repressor activity and the second effector moiety comprises a different transcription repressor activity. In some embodiments, the first effector moiety comprises a transcription repressor activity and the second effector moiety comprises the same transcription repressor activity.

In some embodiments, the first effector moiety comprises, DNMT3a/3l, MQ1, KRAB, G9A, HDAC8, or EZH2 and the second effector moiety comprises DNMT3a/3l, MQ1, KRAB, G9A, HDAC8, or EZH2.

### Linkers

An expression repressor and/or a site-specific disrupting agent may comprise one or more linkers. A linker may connect a targeting moiety to an effector moiety, an effector moiety to another effector moiety, or a targeting moiety to another targeting moiety. A linker may be a chemical bond, e.g., one or more covalent bonds or non-covalent bonds. In some embodiments, a linker is covalent. In some embodiments, a linker is non-covalent. In some embodiments, a linker is a peptide linker. Such a linker may be between 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 amino acids in length, or greater than or equal to 2, 5, 10, 15, 20, 25, or 30 amino acids in length (and optionally up to 50, 40, 30, 25, 20, 15, 10, or 5 amino acids in length). In some embodiments, a linker can be used to space a first moiety from a second moiety, e.g., a targeting moiety from an effector moiety. In some embodiments, for example, a linker can be positioned between a targeting moiety and an effector moiety, e.g., to provide molecular flexibility of secondary and tertiary structures. In some embodiments, a site-specific disrupting agent may comprise a first effector moiety linked to the targeting moiety via a first linker and a second effector moiety linked to the targeting moiety via a second linker. In some embodiments, the first linker has a sequence that is identical to the sequence of the second linker. In some embodiments, the first linker has a sequence that is not identical to the sequence of the second linker. In some embodiments, the first effector moiety is N-terminal of the targeting moiety. In some embodiments, the C-terminal of the targeting moiety. In some embodiments, the C-terminal end of the first effector moiety is linked to the N-terminal end of the targeting moiety via the first linker and the N-terminal end of the second effector moiety is linked to the C-terminal end of the targeting moiety via the second linker.

A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g., polypeptide) from a modulating agent, but sufficiently stable to prevent premature cleavage.

In some embodiments, one or more moieties of an expression repressor described herein are linked with one or more linkers. In some embodiments, one or more moieties of a site-specific disrupting agent described herein are linked with one or more linkers.

As will be known by one of skill in the art, commonly used flexible linkers have sequences consisting primarily of stretches of Gly and Ser residues (“GS” linker). Flexible linkers may be useful for joining domains/moieties that require a certain degree of movement or interaction and may include small, non-polar (e.g., Gly) or polar (e.g., Ser or Thr) amino acids. Incorporation of Ser or Thr can also maintain the stability of a linker in aqueous solutions by forming hydrogen bonds with water molecules, and therefore reduce unfavorable interactions between a linker and moieties/domains.

Rigid linkers are useful to keep a fixed distance between domains/moieties and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains/moieties in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC (SEQ ID NO: 243) results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments.

Examples of molecules suitable for use in linkers described herein include a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more components of a site-specific disrupting agent. Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine, or other hydrophobic residue. Components of a site-specific disrupting agent may be linked using charge-based chemistry, such that a positively charged component of a site-specific disrupting agent is linked to a negative charge of another component.

### Nucleic Acids

In one aspect, the disclosure provides nucleic acid sequences encoding an expression repressor and/or a site-specific disrupting agent, a system, a targeting moiety and/or an effector moiety as described herein. A skilled artisan is aware that the nucleic acid sequences of RNA are identical to the corresponding DNA sequences, except that typically thymine (T) is replaced by uracil (U). It will be understood that when a nucleotide sequence is represented by a DNA sequence (e.g., comprising, A, T, G, C), this disclosure also provides the corresponding RNA sequence (e.g., comprising, A, U, G, C) in which “U” replaces “T.” Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

It will be appreciated by those skilled in the art that due to the degeneracy of the genetic code, a multitude of nucleotide sequences encoding a site-specific disrupting agent comprising DNA-targeting moiety and/or an effector moiety as described herein may be produced, some of which have similarity, e.g., 90%, 95%, 96%, 97%, 98%, or 99% identity to the nucleic acid sequences disclosed herein. For instance, codons AGA, AGG, CGA, CGC, CGG, and CGU all encode the amino acid arginine. Thus, at every position in the nucleic acid of the disclosure where an arginine is specified by a codon, the codon can be altered to any of the corresponding codons described above without altering the encoded polypeptide.

In some embodiments, a nucleic acid sequence encoding an expression repressor comprising a targeting moiety and/or one or more effector moieties may be part or all of a codon-optimized coding region, optimized according to codon usage in mammals, e.g., humans. In some embodiments, a nucleic acid sequence encoding a targeting moiety and/or one or more effector moieties is codon optimized for increasing the protein expression and/or increasing the duration of protein expression. In some embodiments, a protein produced by the codon optimized nucleic acid sequence is at least 1%, at least 2%, at least 5%, at least 10%, at least 15%, at least 20%, at least 30%, at least 40%, or at least 50% higher compared to levels of the protein when encoded by a nucleic acid sequence that is not codon optimized.

In some embodiments, the nucleic acid is an mRNA. In some embodiments, the nucleic acid is monocistronic or polycistronic. In some embodiments, the nucleic acid is monocistronic. In certain embodiments, the nucleic acid is polycistronic (e.g., bi-cistronic, tri-cistronic, tetra-cistronic, etc.). In certain embodiments, the nucleic acid is bi-cistronic. In some embodiments, the nucleic acid is tri-cistronic. In certain embodiments, the nucleic acid is tetra-cistronic.

### Effector Moieties

In some embodiments, a system described herein comprises, or a method described herein comprises the use of, a polypeptide comprising one or more (e.g., one) DNA-targeting moiety and one or more effector moiety, e.g., wherein the effector moiety is or comprises MQ1, e.g., bacterial MQ1, or a functional variant or fragment thereof. In some embodiments, MQ1 is Spiroplasma monobiae MQ1, e.g., MQ1 from strain ATCC 33825 and/or corresponding to Uniprot ID P15840. In some embodiments, MQ1 effector moiety is encoded by a nucleotide sequence of SEQ ID NO: 10. In some embodiments, a nucleotide sequence described herein comprises a sequence of SEQ ID NO: 10 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### MQ1

(SEQ ID NO: 10)
AGCAAGGTGGAGAACAAGACCAAGAAGCTGCGGGTGTTCGAGGCCT
TCGCCGGCATCGGCGCCCAGCGGAAGGCCCTGGAGAAGGTGCGGA
AGGACGAGTACGAGATCGTGGGCCTGGCCGAGTGGTACGTGCCCG
CCATCGTGATGTACCAGGCCATCCACAACAACTTCCACACCAAGC
TGGAGTACAAGAGCGTGAGCCGGGAGGAGATGATCGACTACCTGG
AGAACAAGACCCTGAGCTGGAACAGCAAGAACCCCGTGAGCAACG
GCTACTGGAAGCGGAAGAAGGACGACGAGCTGAAGATCATCTACA
ACGCCATCAAGCTGAGCGAGAAGGAGGGCAACATCTTCGACATCC
GGGACCTGTACAAGCGGACCCTGAAGAACATCGACCTGCTGACCT
ACAGCTTCCCCTGCCAGGACCTGAGCCAGCAGGGCATCCAGAAGG
GCATGAAGCGGGGCAGCGGCACCCGGAGCGGCCTGCTGTGGGAGA
TCGAGCGGGCCCTGGACAGCACCGAGAAGAACGACCTGCCCAAGT
ACCTGCTGATGGAGAACGTGGGCGCCCTGCTGCACAAGAAGAACG
AGGAGGAGCTGAACCAGTGGAAGCAGAAGCTGGAGAGCCTGGGCT
ACCAGAACAGCATCGAGGTGCTGAACGCCGCCGACTTCGGCAGCA
GCCAGGCCCGGCGGCGGGTGTTCATGATCAGCACCCTGAACGAGT
TCGTGGAGCTGCCCAAGGGCGACAAGAAGCCCAAGAGCATCAAGA
AGGTGCTGAACAAGATCGTGAGCGAGAAGGACATCCTGAACAACC
TGCTGAAGTACAACCTGACCGAGTTCAAGAAAACCAAGAGCAACA
TCAACAAGGCCAGCCTGATCGGCTACAGCAAGTTCAACAGCGAGG
GCTACGTGTACGACCCCGAGTTCACCGGCCCCACCCTGACCGCCA
GCGGCGCCAACAGCCGGATCAAGATCAAGGACGGCAGCAACATCC
GGAAGATGAACAGCGACGAGACCTTCCTGTACATCGGCTTCGACA
GCCAGGACGGCAAGCGGGTGAACGAGATCGAGTTCCTGACCGAGA
ACCAGAAGATCTTCGTGTGCGGCAACAGCATCAGCGTGGAGGTGC
TGGAGGCCATCATCGACAAGATCGGCGGC

In some embodiments, MQ1 comprises an amino acid sequence of SEQ ID NO: 11. In some embodiments, MQ1 comprises an amino acid sequence of SEQ ID NO: 12. In some embodiments, an effector domain described herein comprises SEQ ID NO: 11 or 12, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### MQ1

(SEQ ID NO: 11)
MSKVENKTKKLRVFEAFAGIGAQRKALEKVRKDEYEIVGLAEWYV
PAIVMYQAIHNNFHTKLEYKSVSREEMIDYLENKTLSWNSKNPVS
NGYWKRKKDDELKIIYNAIKLSEKEGNIFDIRDLYKRTLKNIDLL
TYSFPCQDLSQQGIQKGMKRGSGTRSGLLWEIERALDSTEKNDLP
KYLLMENVGALLHKKNEEELNQWKQKLESLGYQNSIEVLNAADFG
SSQARRRVFMISTLNEFVELPKGDKKPKSIKKVLNKIVSEKDILN
NLLKYNLTEFKKTKSNINKASLIGYSKFNSEGYVYDPEFTGPTLT
ASGANSRIKIKDGSNIRKMNSDETFLYIGFDSQDGKRVNEIEFLT
ENQKIFVCGNSISVEVLEAIIDKIGG

### MQ1

(SEQ ID NO: 12)
SKVENKTKKLRVFEAFAGIGAQRKALEKVRKDEYEIVGLAEWYVPAIVM
YQAIHNNFHTKLEYKSVSREEMIDYLENKTLSWNSKNPVSNGYWKRKKD
DELKIIYNAIKLSEKEGNIFDIRDLYKRTLKNIDLLTYSFPCQDLSQQG
IQKGMKRGSGTRSGLLWEIERALDSTEKNDLPKYLLMENVGALLHKKNE
EELNQWKQKLESLGYQNSIEVLNAADFGSSQARRRVFMISTLNEFVELP
KGDKKPKSIKKVLNKIVSEKDILNNLLKYNLTEFKKTKSNINKASLIGY
SKFNSEGYVYDPEFTGPTLTASGANSRIKIKDGSNIRKMNSDETFLYIG
FDSQDGKRVNEIEFLTENQKIFVCGNSISVEVLEAIIDKIGG

In some embodiments, MQ1 for use in an expression repressor described herein is a variant, e.g., comprising one or more mutations, relative to wildtype MQ1 (e.g., SEQ ID NO: 11 or SEQ ID NO: 12). In some embodiments, an MQ1 variant comprises one or more amino acid substitutions, deletions, or insertions relative to wildtype MQ1. In some embodiments, an MQ1 variant comprises a K297P substitution. In some embodiments, an MQ1 variant comprises a N299C substitution. In some embodiments, an MQ1 variant comprises a E301Y substitution. In some embodiments, an MQ1 variant comprises a Q147L substitution (e.g., and has reduced DNA methyltransferase activity relative to wildtype MQ1). In some embodiments, an MQ1 variant comprises K297P, N299C, and E301Y substitutions (e.g., and has reduced DNA binding affinity relative to wildtype MQ1). In some embodiments, an MQ1 variant comprises Q147L, K297P, N299C, and E301Y substitutions (e.g., and has reduced DNA methyltransferase activity and DNA binding affinity relative to wildtype MQ1).

In some embodiments, the expression repressor comprises one or more linkers described herein, e.g., connecting a moiety/domain to another moiety/domain. In some embodiments, the expression repressor comprises a targeting moiety that is or comprises a CRISPR/Cas molecule, e.g., comprising a CRISPR/Cas protein, e.g., a dCas9 protein. In some embodiments, the expression repressor is a fusion protein comprising an effector moiety that is or comprises MQ1 and a DNA-targeting moiety that is or comprises a CRISPR/Cas molecule, e.g., comprising a CRISPR/Cas protein, e.g., a dCas9 protein; e.g., a dCas9m4. In some embodiments, the expression repressor comprises an additional moiety described herein. In some embodiments, the expression repressor decreases expression of a target gene or a plurality of target genes (e.g., a target gene or a plurality of target gene described herein). In some embodiments, the expression repressor may be used in methods of modulating, e.g., decreasing, gene expression, methods of treating a condition, or methods of epigenetically modifying a target gene or genomic regulatory element (e.g., transcription control element) described herein. In some embodiments, a system comprises two or more expression repressors.

In some embodiments, a system described herein comprises, or a method described herein comprises the use of, an expression repressor or a polypeptide comprising one or more (e.g., one) targeting moiety and one or more effector moiety, wherein the effector moiety is or comprises Krueppel-associated box (KRAB) domain of Zinc Finger protein 10 e.g., as according to NP 056209.2 or the protein encoded by NM_015394.5 or a functional variant or fragment thereof. In some embodiments, KRAB is a synthetic KRAB construct. In some embodiments, KRAB for use in an expression repressor described herein is a variant, e.g., comprising one or more mutations, relative to wildtype KRAB (e.g., e.g., as according to NP_056209.2 or the protein encoded by NM_015394.5). In some embodiments, a KRAB variant comprises one or more amino acid substitutions, deletions, or insertions relative to wildtype KRAB. In some embodiments, a KRAB variant comprises a L37P substitution. In some embodiments, KRAB comprises an amino acid sequence of SEQ ID NO: 13:

### KRAB

(SEQ ID NO: 13)
DAKSLTAWSRTLVTFKDVFVDFTREEWKLLDTAQQILYRNVMLEN
YKNLVSLGYQLTKPDVILRLEKGEEPWLVEREIHQETHPDSETAF
EIKSSV

In some embodiments, the KRAB effector moiety is encoded by a nucleotide sequence of SEQ ID NO: 14. In some embodiments, a nucleotide sequence described herein comprises a sequence of SEQ ID NO: 14 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### KRAB

(SEQ ID NO: 14)
GACGCCAAGAGCCTGACCGCCTGGAGCCGGACCCTGGTGACCTTC
AAGGACGTGTTCGTGGACTTCACCCGGGAGGAGTGGAAGCTGCTG
GACACCGCCCAGCAGATCCTGTACCGGAACGTGATGCTGGAGAAC
TACAAGAACCTGGTGAGCCTGGGCTACCAGCTGACCAAGCCCGAC
GTGATCCTGCGGCTGGAGAAGGGCGAGGAGCCCTGGCTGGTGGAG
CGGGAGATCCACCAGGAGACCCACCCCGACAGCGAGACCGCCTTC
GAGATCAAGAGCAGCGTG

In some embodiments, KRAB for use in a polypeptide or an expression repressor described herein is a variant, e.g., comprising one or more mutations, relative to the KRAB sequence of SEQ ID NO: 13. In some embodiments, a KRAB variant comprises one or more amino acid substitutions, deletions, or insertions relative to SEQ ID NO: 13.

In some embodiments, the polypeptide or the expression repressor is a fusion protein comprising an effector moiety that is or comprises KRAB and a targeting moiety, e.g., a Zinc Finger domain or Crisper/Cas protein. In some embodiments, the polypeptide or the expression repressor comprises an additional moiety described herein. In some embodiments, the polypeptide or the expression repressor decreases expression of a target gene or a plurality of target genes. In some embodiments, the polypeptide or the expression repressor may be used in methods of modulating, e.g., decreasing, gene expression, methods of treating a condition, or methods of epigenetically modifying a target gene or a plurality of target genes, e.g., a genomic regulatory element (e.g., transcription control element) described herein.

In some embodiments, a system described herein comprises, or a method described herein comprises the use of, an expression repressor or a polypeptide comprising one or more (e.g., one) targeting moiety and one or more effector moiety, wherein the effector moiety is or comprises DNMT3a/3L complex, or a functional variant or fragment thereof. In some embodiments, the DNMT3a/3L complex is a fusion construct. In some embodiments the DNMT3a/3L complex comprises DNMT3A, e.g., human DNMT3A, e.g., as according to NP_072046.2 or the protein encoded by NM_022552.4) or the protein encoded by NM 022552.4 or a functional variant or fragment thereof, e.g., aa 679-912 of human DNMT3A, e.g., as according to NP_072046.2 or the protein encoded by NM_022552.4). In some embodiments the DNMT3a/3L complex comprises human DNMT3L or a functional fragment or variant thereof (e.g., as according to NP_787063.1 or the protein encoded by NM_175867.3 or a functional variant or fragment thereof, e.g., aa 274-386 of human DNMT3L as according to NP_787063.1 or the protein encoded by NM_175867.3). In some embodiments, DNMT3a/3L comprises an amino acid sequence of SEQ ID NO: 15. In some embodiments, an effector moiety described herein comprises SEQ ID NO: 15, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### DNMT3A/3l (h)

(SEQ ID NO: 15)
NHDQEFDPPKVYPPVPAEKRKPIRVLSLFDGIATGLLVLKDLGIQ
VDRYIASEVCEDSITVGMVRHQGKIMYVGDVRSVTQKHIQEWGPF
DLVIGGSPCNDLSIVNPARKGLYEGTGRLFFEFYRLLHDARPKEG
DDRPFFWLFENVVAMGVSDKRDISRFLESNPVMIDAKEVSAAHRA
RYFWGNLPGMNRPLASTVNDKLELQECLEHGRIAKFSKVRTITTR
SNSIKQGKDQHFPVFMNEKEDILWCTEMERVFGFPVHYTDVSNMS
RLARQRLLGRSWSVPVIRHLFAPLKEYFACVSSGNSNANSRGPSF
SSGLVPLSLRGSHMNPLEMFETVPVWRRQPVRVLSLFEDIKKELT
SLGFLESGSDPGQLKHVVDVTDTVRKDVEEWGPFDLVYGATPPLG
HTCDRPPSWYLFQFHRLLQYARPKPGSPRPFFWMFVDNLVLNKED
LDVASRFLEMEPVTIPDVHGGSLQNAVRVWSNIPAIRSRHWALVS
EEELSLLAQNKQSSKLAAKWPTKLVKNCFLPLREYFKYFSTELTS
SL

In some embodiments, DNMT3a/3L is encoded by a nucleotide sequence of SEQ ID NO: 16. In some embodiments, a nucleic acid described herein comprises a sequence of SEQ ID NO: 16 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### DNMT3A/3l (h)

(SEQ ID NO: 16)
AACCACGACCAGGAGTTCGACCCCCCCAAGGTGTACCCCCCCGTG
CCCGCCGAGAAGCGGAAGCCCATCCGGGTGCTGAGCCTGTTCGAC
GGCATCGCCACCGGCCTGCTGGTGCTGAAGGACCTGGGCATCCAG
GTGGACCGGTACATCGCCAGCGAGGTGTGCGAGGACAGCATCACC
GTGGGCATGGTGCGGCACCAGGGCAAGATCATGTACGTGGGCGAC
GTGCGGAGCGTGACCCAGAAGCACATCCAGGAGTGGGGCCCCTTC
GACCTGGTGATCGGCGGCAGCCCCTGCAACGACCTGAGCATCGTG
AACCCCGCCCGGAAGGGCCTGTACGAGGGCACCGGCCGGCTGTTC
TTCGAGTTCTACCGGCTGCTGCACGACGCCCGGCCCAAGGAGGGC
GACGACCGGCCCTTCTTCTGGCTGTTCGAGAACGTGGTGGCCATG
GGCGTGAGCGACAAGCGGGACATCAGCCGGTTCCTGGAGAGCAAC
CCCGTGATGATCGACGCCAAGGAGGTGAGCGCCGCCCACCGGGCC
CGGTACTTCTGGGGCAACCTGCCCGGCATGAACCGGCCCCTGGCC
AGCACCGTGAACGACAAGCTGGAGCTGCAGGAGTGCCTGGAGCAC
GGCCGGATCGCCAAGTTCAGCAAGGTGCGGACCATCACCACCCGG
AGCAACAGCATCAAGCAGGGCAAGGACCAGCACTTCCCCGTGTTC
ATGAACGAGAAGGAGGACATCCTGTGGTGCACCGAGATGGAGCGG
GTGTTCGGCTTCCCCGTGCACTACACCGACGTGAGCAACATGAGC
CGGCTGGCCCGGCAGCGGCTGCTGGGCCGGAGCTGGAGCGTGCCC
GTGATCCGGCACCTGTTCGCCCCCCTGAAGGAGTACTTCGCCTGC
GTGAGCAGCGGCAACAGCAACGCCAACAGCCGGGGCCCCAGCTTC
AGCAGCGGCCTGGTGCCCCTGAGCCTGCGGGGCAGCCACATGAAT
CCTCTGGAGATGTTCGAGACAGTGCCCGTGTGGAGAAGGCAACCC
GTGAGGGTGCTGAGCCTCTTCGAGGACATTAAGAAGGAGCTGACC
TCTCTGGGCTTTCTGGAATCCGGCAGCGACCCCGGCCAGCTGAAA
CACGTGGTGGACGTGACCGACACAGTGAGGAAGGACGTGGAAGAG
TGGGGCCCCTTTGACCTCGTGTATGGAGCCACACCTCCTCTCGGC
CACACATGCGATAGGCCTCCCAGCTGGTATCTCTTCCAGTTCCAC
AGACTGCTCCAGTACGCCAGACCTAAGCCCGGCAGCCCCAGACCC
TTCTTCTGGATGTTCGTGGACAATCTGGTGCTGAACAAGGAGGAT
CTGGATGTGGCCAGCAGATTTCTGGAGATGGAACCCGTGACAATC
CCCGACGTGCATGGCGGCTCTCTGCAGAACGCCGTGAGAGTGTGG
TCCAACATCCCCGCCATTAGAAGCAGACACTGGGCTCTGGTGAGC
GAGGAGGAACTGTCTCTGCTGGCCCAGAATAAGCAGTCCTCCAAG
CTGGCCGCCAAGTGGCCCACCAAGCTGGTGAAGAACTGCTTTCTG
CCTCTGAGGGAGTATTTCAAGTATTTCAGCACCGAACTGACCAGC
AGCCTG

In some embodiments, a system described herein comprises, or a method described herein comprises the use of, an expression repressor or a polypeptide comprising one or more (e.g., one) targeting moiety and one or more effector moiety, wherein the effector moiety is or comprises EZH2, e.g., as according to NP-004447.2 or NP_001190176.1 2 or the protein encoded by NM_004456.5 or NM_001203247.2 or a functional variant or fragment thereof. In some embodiments, EZH2 for use in an expression repressor described herein is a variant, e.g., comprising one or more mutations, relative to EZH2, e.g., as according to NP-004447.2 or NP 001190176.1 2 or the protein encoded by NM 004456.5 or NM_001203247.2. In some embodiments, an EZH2 variant comprises one or more amino acid substitutions, deletions, or insertions relative to wildtype EZH2. In some embodiments, EZH2 comprises an amino acid sequence of SEQ ID NO: 17:

### EZH2

(SEQ ID NO: 17)
GQTGKKSEKGPVCWRKRVKSEYMRLRQLKRFRRADEVKSMFSSNR
QKILERTEILNQEWKQRRIQPVHILTSVSSLRGTRECSVTSDLDF
PTQVIPLKTLNAVASVPIMYSWSPLQQNFMVEDETVLHNIPYMGD
EVLDQDGTFIEELIKNYDGKVHGDRECGFINDEIFVELVNALGQY
NDDDDDDDGDDPEEREEKQKDLEDHRDDKESRPPRKFPSDKIFEA
ISSMFPDKGTAEELKEKYKELTEQQLPGALPPECTPNIDGPNAKS
VQREQSLHSFHTLFCRRCFKYDCFLHPFHATPNTYKRKNTETALD
NKPCGPQCYQHLEGAKEFAAALTAERIKTPPKRPGGRRRGRLPNN
SSRPSTPTINVLESKDTDSDREAGTETGGENNDKEEEEKKDETSS
SSEANSRCQTPIKMKPNIEPPENVEWSGAEASMFRVLIGTYYDNF
CAIARLIGTKTCRQVYEFRVKESSIIAPAPAEDVDTPPRKKKRKH
RLWAAHCRKIQLKKDGSSNHVYNYQPCDHPRQPCDSSCPCVIAQN
FCEKFCQCSSECQNRFPGCRCKAQCNTKQCPCYLAVRECDPDLCL
TCGAADHWDSKNVSCKNCSIQRGSKKHLLLAPSDVAGWGIFIKDP
VQKNEFISEYCGEIISQDEADRRGKVYDKYMCSFLFNLNNDFVVD
ATRKGNKIRFANHSVNPNCYAKVMMVNGDHRIGIFAKRAIQTGEE
LFFDYRYSQADALKYVGIEREMEIP

In some embodiments, the EZH2 effector moiety is encoded by a nucleotide sequence of SEQ ID NO: 18. In some embodiments, a nucleotide sequence described herein comprises a sequence of SEQ ID NO: 18 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### EZH2

(SEQ ID NO: 18)
GGCCAGACCGGCAAGAAGAGCGAGAAGGGCCCCGTGTGCTGGCGG
AAGCGGGTGAAGAGCGAGTACATGCGGCTGCGGCAGCTGAAGCGG
TTCCGGCGGGCCGACGAGGTGAAGAGCATGTTCAGCAGCAACCGG
CAGAAGATCCTGGAGCGGACCGAGATCCTGAACCAGGAGTGGAAG
CAGCGGCGAATCCAGCCCGTGCACATCCTGACCAGCGTGAGCAGC
CTGCGGGGCACCCGGGAGTGCAGCGTGACCAGCGACCTGGACTTC
CCCACCCAGGTGATCCCCCTAAAGACCCTGAACGCCGTGGCCAGC
GTGCCCATCATGTACAGCTGGAGCCCCCTGCAGCAGAACTTCATG
GTGGAGGACGAGACCGTGCTGCACAACATCCCCTACATGGGCGAC
GAGGTGCTGGACCAGGACGGCACCTTCATCGAGGAGCTGATCAAG
AACTACGACGGCAAGGTGCACGGCGACCGGGAGTGCGGCTTCATC
AACGACGAGATCTTCGTGGAGCTGGTGAACGCCCTGGGCCAGTAC
AACGACGACGACGACGACGACGACGGCGACGACCCCGAGGAGCGG
GAGGAGAAGCAGAAGGACCTGGAGGACCACCGGGACGACAAGGAG
AGCCGGCCCCCCCGGAAGTTCCCCAGCGACAAGATCTTCGAGGCC
ATCAGCAGCATGTTCCCCGACAAGGGCACCGCCGAGGAGCTGAAG
GAGAAGTACAAGGAGCTGACCGAGCAGCAGCTGCCCGGCGCCCTG
CCCCCCGAGTGCACCCCCAACATCGACGGCCCCAACGCCAAGAGC
GTGCAGCGGGAGCAGAGCCTGCACAGCTTCCACACCCTGTTCTGC
CGGCGGTGCTTCAAGTACGACTGCTTCCTGCACCCCTTCCACGCC
ACCCCCAACACCTACAAGCGGAAGAACACCGAGACCGCCCTGGAC
AACAAGCCCTGCGGCCCCCAGTGCTACCAGCACCTGGAGGGCGCC
AAGGAGTTCGCCGCCGCCCTGACCGCCGAGCGGATCAAGACCCCC
CCCAAGCGGCCCGGCGGCCGGCGGCGGGGCCGGCTGCCCAACAAC
AGCAGCCGGCCCAGCACCCCCACCATCAACGTGCTGGAGAGCAAG
GACACCGACAGCGACCGGGAGGCCGGCACCGAGACCGGCGGCGAG
AACAACGACAAGGAGGAGGAGGAGAAGAAGGACGAGACCAGCAGC
AGCAGCGAGGCCAACAGCCGGTGCCAGACCCCCATCAAGATGAAG
CCCAACATCGAGCCCCCCGAGAACGTGGAGTGGAGCGGCGCCGAG
GCCAGCATGTTCCGGGTGCTGATCGGCACCTACTACGACAACTTC
TGCGCCATCGCCCGGCTGATCGGCACCAAGACCTGCCGGCAGGTG
TACGAGTTCCGGGTGAAGGAGAGCAGCATCATCGCCCCCGCCCCC
GCCGAGGACGTGGACACCCCCCCCCGGAAGAAGAAGCGGAAGCAC
CGGCTGTGGGCCGCCCACTGCCGGAAGATCCAGCTGAAGAAGGAC
GGCAGCAGCAACCACGTGTACAACTACCAGCCCTGCGACCACCCC
CGGCAGCCCTGCGACAGCAGCTGCCCCTGCGTGATCGCCCAGAAC
TTCTGCGAGAAGTTCTGCCAGTGCAGCAGCGAGTGCCAGAACCGG
TTCCCCGGCTGCCGGTGCAAGGCCCAGTGCAACACCAAGCAGTGC
CCCTGCTACCTGGCCGTGCGGGAGTGCGACCCCGACCTGTGCCTG
ACCTGCGGCGCCGCCGACCACTGGGACAGCAAGAACGTGAGCTGC
AAGAACTGCAGCATCCAGCGGGGCAGCAAGAAGCACCTGCTGCTG
GCCCCCAGCGACGTGGCCGGCTGGGGCATCTTCATCAAGGACCCC
GTGCAGAAGAACGAGTTCATCAGCGAGTACTGCGGCGAGATCATC
AGCCAGGACGAGGCCGACCGGCGGGGCAAGGTGTACGACAAGTAC
ATGTGCAGCTTCCTGTTCAACCTGAACAACGACTTCGTGGTGGAC
GCCACCCGGAAGGGCAACAAGATCCGGTTCGCCAACCACAGCGTG
AACCCCAACTGCTACGCCAAGGTGATGATGGTGAACGGCGACCAC
CGGATCGGCATCTTCGCCAAGCGGGCCATCCAGACCGGCGAGGAG
CTGTTCTTCGACTACCGGTACAGCCAGGCCGACGCCCTGAAGTAC
GTGGGCATCGAGCGGGAGATGGAGATCCCC

In some embodiments, EZH2 for use in a polypeptide or expression repressor described herein is a variant, e.g., comprising one or more mutations, relative to the EZH2 sequence of SEQ ID NO: 17. In some embodiments, an EZH2 variant comprises one or more amino acid substitutions, deletions, or insertions relative to SEQ ID NO: 17.

In some embodiments, the polypeptide or the expression repressor is a fusion protein comprising an effector moiety that is or comprises EZH2 and a targeting moiety. In some embodiments, the polypeptide or the expression repressor comprises an additional moiety described herein. In some embodiments, the polypeptide or the expression repressor decreases expression of a target gene or a plurality of target genes. In some embodiments, the polypeptide or the expression repressor may be used in methods of modulating, e.g., decreasing, gene expression, methods of treating a condition, or methods of epigenetically modifying a target gene or a plurality of target genes, e.g., a genomic regulatory element (e.g., transcription control element) described herein.

In some embodiments, a system described herein comprises, or a method described herein comprises the use of, an expression repressor or a polypeptide comprising one or more (e.g., one) targeting moiety and one or more effector moiety, wherein the effector moiety is or comprises HDAC8, e.g., as according to NP_001159890 or NP_060956.1 or the protein encoded by NM_001166418 or NM_018486.3 or a functional variant or fragment thereof. In some embodiments, HDAC8 comprises an amino acid sequence of SEQ ID NO: 19:

### HDAC8

(SEQ ID NO: 19)
EEPEEPADSGQSLVPVYIYSPEYVSMCDSLAKIPKRASMVHSLIE
AYALHKQMRIVKPKVASMEEMATFHTDAYLQHLQKVSQEGDDDHP
DSIEYGLGYDCPATEGIFDYAAAIGGATITAAQCLIDGMCKVAIN
WSGGWHHAKKDEASGFCYLNDAVLGILRLRRKFERILYVDLDLHH
GDGVEDAFSFTSKVMTVSLHKFSPGFFPGTGDVSDVGLGKGRYYS
VNVPIQDGIQDEKYYQICESVLKEVYQAFNPKAVVLQLGADTIAG
DPMCSFNMTPVGIGKCLKYILQWQLATLILGGGGYNLANTARCWT
YLTGVILGKTLSSEIPDHEFFTAYGPDYVLEITPSCRPDRNEPHR
IQQILNYIKGNLKHVV

In some embodiments, the HDAC8 effector moiety is encoded by a nucleotide sequence of SEQ ID NO: 66. In some embodiments, a nucleotide sequence described herein comprises a sequence of SEQ ID NO: 66 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### HDAC8

(SEQ ID NO: 66)
GAGGAGCCCGAGGAGCCCGCCGATAGCGGACAATCTCTGGTGCCC
GTCTACATCTACAGCCCCGAATATGTGAGCATGTGTGATTCCCTC
GCCAAGATCCCTAAGAGAGCCAGCATGGTGCATTCTCTGATCGAG
GCCTACGCTCTGCATAAGCAAATGAGGATCGTGAAGCCCAAGGTC
GCCAGCATGGAAGAGATGGCCACCTTTCACACCGATGCCTACCTC
CAACATCTCCAGAAGGTGTCCCAAGAGGGCGACGACGACCACCCC
GACTCCATTGAGTACGGACTGGGCTATGATTGCCCCGCCACCGAG
GGCATCTTTGACTATGCCGCCGCTATCGGCGGAGCTACCATCACA
GCCGCCCAGTGTCTGATTGATGGCATGTGCAAGGTCGCCATCAAC
TGGTCCGGAGGCTGGCATCATGCCAAGAAGGATGAGGCCTCCGGC
TTCTGTTATCTGAATGACGCCGTGCTGGGCATTCTGAGACTGAGG
AGGAAATTCGAGAGGATTCTGTACGTGGATCTGGATCTGCATCAC
GGAGATGGAGTCGAAGATGCCTTCAGCTTCACCAGCAAGGTGATG
ACAGTCTCTCTGCACAAGTTCTCCCCCGGCTTCTTTCCCGGAACC
GGCGACGTGTCCGACGTGGGACTGGGCAAGGGAAGGTACTACAGC
GTGAACGTGCCCATTCAAGACGGCATCCAAGACGAGAAGTACTAC
CAGATCTGCGAGTCCGTGCTCAAGGAGGTCTACCAAGCCTTCAAT
CCTAAGGCTGTCGTGCTCCAACTGGGAGCTGATACCATTGCTGGC
GATCCCATGTGCAGCTTCAATATGACACCCGTCGGAATCGGCAAG
TGCCTCAAGTACATCCTCCAGTGGCAGCTCGCCACCCTCATTCTC
GGAGGAGGCGGATACAATCTGGCTAATACCGCCAGATGCTGGACC
TATCTGACCGGCGTGATTCTGGGCAAAACACTGAGCAGCGAAATC
CCCGACCACGAGTTTTTCACCGCTTACGGCCCCGACTACGTGCTG
GAGATCACCCCCAGCTGCAGACCCGATAGAAACGAACCCCATAGA
ATCCAGCAAATTCTGAACTATATCAAGGGCAACCTCAAGCACGTC
GTG

In some embodiments, the HDAC8 for use in a polypeptide or an expression repressor described herein is a variant, e.g., comprising one or more mutations, relative to the HDAC8 sequence of SEQ ID NO: 19. In some embodiments, an HDAC8 variant comprises one or more amino acid substitutions, deletions, or insertions relative to SEQ ID NO: 19.

In some embodiments, the polypeptide or the expression repressor is a fusion protein comprising an effector moiety that is or comprises HDAC8 and a targeting moiety. In some embodiments, the polypeptide or the expression repressor comprises an additional moiety described herein. In some embodiments, the polypeptide or the expression repressor decreases expression of a target gene or a plurality of target genes. In some embodiments, the polypeptide or the expression repressor may be used in methods of modulating, e.g., decreasing, gene expression, methods of treating a condition, or methods of epigenetically modifying a target gene or a plurality of target genes, e.g., a genomic regulatory element (e.g., transcription control element) described herein.

In some embodiments, a system described herein comprises, or a method described herein comprises the use of, an expression repressor or a polypeptide comprising one or more (e.g., one) targeting moiety and one or more effector moiety, wherein the effector moiety is or comprises G9A e.g., as according to NP_001350618.1 or the protein encoded by NM_001363689.1 or a functional variant or fragment thereof, e.g., aa967-1250 of comprises G9A e.g., as according to NP_001350618.1 or the protein encoded by NM_001363689.1. In some embodiments, G9A comprises an amino acid sequence of SEQ ID NO: 67:

### G9A

(SEQ ID NO: 67)
GNRAIRTEKIICRDVARGYENVPIPCVNGVDGEPCPEDYKYISEN
CETSTMNIDRNITHLQHCTCVDDCSSSNCLCGQLSIRCWYDKDGR
LLQEFNKIEPPLIFECNQACSCWRNCKNRVVQSGIKVRLQLYRTA
KMGWGVRALQTIPQGTFICEYVGELISDAEADVREDDSYLFDLDN
KDGEVYCIDARYYGNISRFINHLCDPNIIPVRVFMLHQDLRFPRI
AFFSSRDIRTGEELGFDYGDRFWDIKSKYFTCQCGSEKCKHSAEA
IALEQSRLARLD

In some embodiments, the G9A effector moiety is encoded by a nucleotide sequence of SEQ ID NO: 68. In some embodiments, a nucleotide sequence described herein comprises a sequence of SEQ ID NO: 68 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### G9A

(SEQ ID NO: 68)
GGAAATAGGGCTATCAGAACCGAGAAGATCATCTGTAGGGACGTG
GCTAGAGGCTACGAGAACGTGCCCATTCCTTGCGTGAATGGCGTG
GATGGCGAACCTTGCCCCGAGGACTACAAATACATCTCCGAGAAC
TGCGAAACCAGCACAATGAACATCGACAGAAACATCACCCACCTC
CAGCACTGCACATGTGTGGATGACTGCTCCTCCAGCAACTGTCTG
TGCGGCCAGCTCTCCATCAGATGCTGGTACGACAAGGACGGCAGA
CTGCTGCAAGAGTTCAACAAGATCGAACCCCCTCTCATCTTCGAG
TGTAACCAAGCTTGCAGCTGCTGGAGAAACTGCAAGAATAGAGTG
GTCCAGAGCGGCATCAAGGTGAGACTGCAACTGTACAGAACCGCC
AAGATGGGATGGGGAGTGAGGGCTCTGCAAACCATTCCCCAAGGC
ACCTTCATCTGCGAATACGTGGGCGAACTGATCTCCGACGCCGAA
GCTGACGTGAGAGAGGACGACAGCTATCTCTTCGATCTGGACAAT
AAGGACGGCGAGGTGTACTGCATCGACGCTAGATATTACGGCAAC
ATCTCTAGATTCATCAACCACCTCTGCGATCCCAACATCATTCCC
GTGAGGGTGTTCATGCTGCACCAAGATCTGAGGTTCCCTAGAATC
GCCTTCTTCAGCTCTAGAGACATCAGAACCGGCGAGGAGCTGGGC
TTCGATTACGGCGATAGATTCTGGGACATCAAGTCCAAGTACTTC
ACATGCCAGTGCGGCAGCGAGAAGTGTAAGCACAGCGCTGAGGCC
ATTGCTCTGGAGCAGTCTAGACTGGCCAGACTGGAT

In some embodiments, G9A for use in a polypeptide or an expression repressor described herein is a variant, e.g., comprising one or more mutations, relative to the G9A sequence of SEQ ID NO: 67. In some embodiments, an G9A variant comprises one or more amino acid substitutions, deletions, or insertions relative to SEQ ID NO: 67.

In some embodiments, the polypeptide or the expression repressor is a fusion protein comprising an effector moiety that is or comprises G9A and a targeting moiety. In some embodiments, the polypeptide or the expression repressor comprises an additional moiety described herein. In some embodiments, the polypeptide or the expression repressor decreases expression of a target gene or a plurality of target genes. In some embodiments, the polypeptide or the expression repressor may be used in methods of modulating, e.g., decreasing, gene expression, methods of treating a condition, or methods of epigenetically modifying a target gene or a plurality of target genes, e.g., a genomic regulatory element (e.g., transcription control element) described herein.

In some embodiments, a nucleic acid sequence encoding a site-specific disrupting agent comprising a targeting moiety and/or one or more effector moieties may be part or all of a codon-optimized coding region, optimized according to codon usage in mammals, e.g., humans. In some embodiments, a nucleic acid sequence encoding a targeting moiety and/or one or more effector moieties is codon optimized for increasing the protein expression and/or increasing the duration of protein expression. In some embodiments, a protein produced by the codon optimized nucleic acid sequence is at least 1%, at least 2%, at least 5%, at least 10%, at least 15%, at least 20%, at least 30%, at least 40%, or at least 50% higher compared to levels of the protein when encoded by a nucleic acid sequence that is not codon optimized.

In some embodiments, a system described herein comprises, or a method described herein comprises the use of, a polypeptide comprising one or more (e.g., one) DNA-targeting moiety and one or more effector moiety, e.g., wherein the effector moiety is or comprises MQ1, e.g., bacterial MQ1, or a functional variant or fragment thereof. In some embodiments, MQ1 is Spiroplasma monohiae MQ1, e.g., MQ1 from strain ATCC 33825 and/or corresponding to Uniprot ID P15840. In some embodiments, MQ1 effector moiety is encoded by a nucleotide sequence of SEQ ID NO: 10. In some embodiments, a nucleotide sequence described herein comprises a sequence of SEQ ID NO: 10 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, MQ1 comprises an amino acid sequence of SEQ ID NO: 11. In some embodiments, MQ1 comprises an amino acid sequence of SEQ ID NO: 12. In some embodiments, an effector domain described herein comprises SEQ ID NO: 11 or 12, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, MQ1 for use in a site-specific disrupting agent described herein is a variant, e.g., comprising one or more mutations, relative to wildtype MQ1 (e.g., SEQ ID NO: 11 or SEQ ID NO: 12). In some embodiments, an MQ1 variant comprises one or more amino acid substitutions, deletions, or insertions relative to wildtype MQ1. In some embodiments, an MQ1 variant comprises a K297P substitution. In some embodiments, an MQ1 variant comprises a N299C substitution. In some embodiments, an MQ1 variant comprises a E301Y substitution. In some embodiments, an MQ1 variant comprises a Q147L substitution (e.g., and has reduced DNA methyltransferase activity relative to wildtype MQ1). In some embodiments, an MQ1 variant comprises K297P, N299C, and E301Y substitutions (e.g., and has reduced DNA binding affinity relative to wildtype MQ1). In some embodiments, an MQ1 variant comprises Q147L, K297P, N299C, and E301Y substitutions (e.g., and has reduced DNA methyltransferase activity and DNA binding affinity relative to wildtype MQ1).

In some embodiments, the site-specific disrupting agent comprises one or more linkers described herein, e.g., connecting a moiety/domain to another moiety/domain. In some embodiments, the site-specific disrupting agent comprises a targeting moiety that is or comprises a CRISPR/Cas molecule, e.g., comprising a CRISPR/Cas protein, e.g., a dCas9 protein. In some embodiments, the site-specific disrupting agent is a fusion protein comprising an effector moiety that is or comprises MQ1 and a DNA-targeting moiety that is or comprises a CRISPR/Cas molecule, e.g., comprising a CRISPR/Cas protein, e.g., a dCas9 protein; e.g., a dCas9m4. In some embodiments, the site-specific disrupting agent comprises an additional moiety described herein. In some embodiments, the site-specific disrupting agent decreases expression of a target gene or a plurality of target genes (e.g., a target gene or a plurality of target gene described herein). In some embodiments, the site-specific disrupting agent may be used in methods of modulating, e.g., decreasing, gene expression, methods oftreating a condition, or methods of epigenetically modifying a target gene or genomic regulatory element (e.g., transcription control element) described herein. In some embodiments, a system comprises two or more site-specific disrupting agents.

In some embodiments, a system described herein comprises, or a method described herein comprises the use of, a site-specific disrupting agent or a polypeptide comprising one or more (e.g., one) targeting moiety and one or more effector moiety, wherein the effector moiety is or comprises Krueppel-associated box (KRAB) domain of Zinc Finger protein 10 e.g., as according to NP 056209.2 or the protein encoded by NM_015394.5 or a functional variant or fragment thereof. In some embodiments, KRAB is a synthetic KRAB construct. In some embodiments, KRAB for use in a site-specific disrupting agent described herein is a variant, e.g., comprising one or more mutations, relative to wildtype KRAB (e.g., e.g., as according to NP_056209.2 or the protein encoded by NM_015394.5). In some embodiments, a KRAB variant comprises one or more amino acid substitutions, deletions, or insertions relative to wildtype KRAB. In some embodiments, a KRAB variant comprises a L37P substitution. In some embodiments, KRAB comprises an amino acid sequence of SEQ ID NO: 13.

In some embodiments, the KRAB effector moiety is encoded by a nucleotide sequence of SEQ ID NO: 14. In some embodiments, a nucleotide sequence described herein comprises a sequence of SEQ ID NO: 14 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, KRAB for use in a polypeptide or a site-specific disrupting agent described herein is a variant, e.g., comprising one or more mutations, relative to the KRAB sequence of SEQ ID NO: 13. In some embodiments, a KRAB variant comprises one or more amino acid substitutions, deletions, or insertions relative to SEQ ID NO: 13.

In some embodiments, the polypeptide or the site-specific disrupting agent is a fusion protein comprising an effector moiety that is or comprises KRAB and a targeting moiety, e.g., a CRISPR/Cas protein. In some embodiments, the polypeptide or the site-specific disrupting agent comprises an additional moiety described herein. In some embodiments, the polypeptide or the site-specific disrupting agent decreases expression of a target gene or a plurality of target genes. In some embodiments, the polypeptide or the site-specific disrupting agent may be used in methods of modulating, e.g., decreasing, gene expression, methods of treating a condition, or methods of epigenetically modifying a target gene or a plurality of target genes, e.g., a genomic regulatory element (e.g., transcription control element) described herein.

In some embodiments, a system described herein comprises, or a method described herein comprises the use of, a site-specific disrupting agent or a polypeptide comprising one or more (e.g., one) targeting moiety and one or more effector moiety, wherein the effector moiety is or comprises DNMT3a/3L complex, or a functional variant or fragment thereof. In some embodiments, the DNMT3a/3L complex is a fusion construct. In some embodiments the DNMT3a/3L complex comprises DNMT3A, e.g., human DNMT3A, e.g., as according to NP_072046.2 or the protein encoded by NM_022552.4) or the protein encoded by NM_022552.4 or a functional variant or fragment thereof, e.g., aa 679-912 of human DNMT3A, e.g., as according to NP_072046.2 or the protein encoded by NM_022552.4). In some embodiments the DNMT3a/3L complex comprises human DNMT3L or a functional fragment or variant thereof (e.g., as according to NP_787063.1 or the protein encoded by NM_175867.3 or a functional variant or fragment thereof, e.g., aa 274-386 of human DNMT3L as according to NP 787063.1 or the protein encoded by NM_175867.3). In some embodiments, DNMT3a/3L comprises an amino acid sequence of SEQ ID NO: 15. In some embodiments, an effector moiety described herein comprises SEQ ID NO: 15, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, DNMT3a/3L is encoded by a nucleotide sequence of SEQ ID NO: 16. In some embodiments, a nucleic acid described herein comprises a sequence of SEQ ID NO: 16 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, a system described herein comprises, or a method described herein comprises the use of, a site-specific disrupting agent or a polypeptide comprising one or more (e.g., one) targeting moiety and one or more effector moiety, wherein the effector moiety is or comprises EZH2, e.g., as according to NP-004447.2 or NP_001190176.1 2 or the protein encoded by NM_004456.5 or NM_001203247.2 or a functional variant or fragment thereof. In some embodiments, MQ1 for use in a site-specific disrupting agent described herein is a variant, e.g., comprising one or more mutations, relative to EZH2, e.g., as according to NP-004447.2 or NP_001190176.1 2 or the protein encoded by NM_004456.5 or NM_001203247.2. In some embodiments, an EZH2 variant comprises one or more amino acid substitutions, deletions, or insertions relative to wildtype EZH2. In some embodiments, EZH2 comprises an amino acid sequence of SEQ ID NO: 17.

In some embodiments, the EZH2 effector moiety is encoded by a nucleotide sequence of SEQ ID NO: 18. In some embodiments, a nucleotide sequence described herein comprises a sequence of SEQ ID NO: 18 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, EZH2 for use in a polypeptide or a site-specific disrupting agent described herein is a variant, e.g., comprising one or more mutations, relative to the EZH2 sequence of SEQ ID NO: 17. In some embodiments, an EZH2 variant comprises one or more amino acid substitutions, deletions, or insertions relative to SEQ ID NO: 17.

In some embodiments, the polypeptide or the site-specific disrupting agent is a fusion protein comprising an effector moiety that is or comprises EZH2 and a targeting moiety. In some embodiments, the polypeptide or the site-specific disrupting agent comprises an additional moiety described herein. In some embodiments, the polypeptide or the site-specific disrupting agent decreases expression of a target gene or a plurality of target genes. In some embodiments, the polypeptide or the site-specific disrupting agent may be used in methods of modulating, e.g., decreasing, gene expression, methods of treating a condition, or methods of epigenetically modifying a target gene or a plurality of target genes, e.g., a genomic regulatory element (e.g., transcription control element) described herein.

In some embodiments, a system described herein comprises, or a method described herein comprises the use of, a site-specific disrupting agent or a polypeptide comprising one or more (e.g., one) targeting moiety and one or more effector moiety, wherein the effector moiety is or comprises HDAC8, e.g., as according to NP_001159890 or NP_060956.1 or the protein encoded by NM_001166418 or NM_018486.3 or a functional variant or fragment thereof. In some embodiments, HDAC8 comprises an amino acid sequence of SEQ ID NO: 19.

In some embodiments, the HDAC8 effector moiety is encoded by a nucleotide sequence of SEQ ID NO: 66. In some embodiments, a nucleotide sequence described herein comprises a sequence of SEQ ID NO: 66 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, the HDAC8 for use in a polypeptide or a site-specific disrupting agent described herein is a variant, e.g., comprising one or more mutations, relative to the HDAC8 sequence of SEQ ID NO: 19. In some embodiments, an HDAC8 variant comprises one or more amino acid substitutions, deletions, or insertions relative to SEQ ID NO: 19.

In some embodiments, the polypeptide or the site-specific disrupting agent is a fusion protein comprising an effector moiety that is or comprises HDAC8 and a targeting moiety. In some embodiments, the polypeptide or the site-specific disrupting agent comprises an additional moiety described herein. In some embodiments, the polypeptide or the site-specific disrupting agent decreases expression of a target gene or a plurality of target genes. In some embodiments, the polypeptide or the site-specific disrupting agent may be used in methods of modulating, e.g., decreasing, gene expression, methods of treating a condition, or methods of epigenetically modifying a target gene or a plurality of target genes. e.g., a genomic regulatory element (e.g., transcription control element) described herein.

In some embodiments, a system described herein comprises, or a method described herein comprises the use of, a site-specific disrupting agent or a polypeptide comprising one or more (e.g., one) targeting moiety and one or more effector moiety, wherein the effector moiety is or comprises G9A e.g., as according to NP_001350618.1 or the protein encoded by NM_001363689.1 or a functional variant or fragment thereof, e.g., aa967-1250 of comprises G9A e.g., as according to NP_001350618.1 or the protein encoded by NM_001363689.1. In some embodiments, G9A comprises an amino acid sequence of SEQ ID NO: 67.

In some embodiments, the G9A effector moiety is encoded by a nucleotide sequence of SEQ ID NO: 68. In some embodiments, a nucleotide sequence described herein comprises a sequence of SEQ ID NO: 68 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, G9A for use in a polypeptide or a site-specific disrupting agent described herein is a variant, e.g., comprising one or more mutations, relative to the G9A sequence of SEQ ID NO: 67. In some embodiments, a G9A variant comprises one or more amino acid substitutions, deletions, or insertions relative to SEQ ID NO: 67.

In some embodiments, the polypeptide or the site-specific disrupting agent is a fusion protein comprising an effector moiety that is or comprises G9A and a targeting moiety. In some embodiments, the polypeptide or the site-specific disrupting agent comprises an additional moiety described herein. In some embodiments, the polypeptide or the site-specific disrupting agent decreases expression of a target gene or a plurality of target genes. In some embodiments, the polypeptide or the site-specific disrupting agent may be used in methods of modulating, e.g., decreasing, gene expression, methods of treating a condition, or methods of epigenetically modifying a target gene or a plurality of target genes, e.g., a genomic regulatory element (e.g., transcription control element) described herein.

### Systems

Systems of the present disclosure may comprise two or more expression repressors. In some embodiments, an expression repressor system comprises 2, 3, 4, 5, 6, 7, 8, 9, 10, 11, 12, or more, expression repressors (and optionally no more than 15, 14, 13, 12, 11, 10, 9, 8, 7, 6, 5, 4, 3, or 2). In some embodiments, system targets two or more different sequences (e.g., a 1st and 2nd, 3rd, 4th, 5th, 6th, 7th, 8th, 9th, 10th, 11th, 12th, and/or further DNA sequence, and optionally no more than a 20th, 19th, 18th, 17th, 16th, 15th, 14th, 13th, 12th, 11th, 10th, 9th, 8th, 6th, 5th, 4th, 3rd, or 2nd sequence). In some embodiments, system comprises a plurality of expression repressors, wherein each member of the plurality of expression repressors does not detectably bind, e.g., does not bind, to another member of the plurality of expression repressors. In some embodiments, system comprises a first expression repressor and a second expression repressor, wherein the first expression repressor does not detectably bind, e.g., does not bind, to the second expression repressor.

In some embodiments, a system of the present disclosure comprises two or more expression repressors, wherein the expression repressors are present together in a composition, pharmaceutical composition, or mixture. In some embodiments, a system of the present disclosure comprises two or more expression repressors, wherein one or more expression repressors is not admixed with at least one other expression repressor. In some embodiments, a system may comprise a first expression repressor and a second expression repressor, wherein the presence of the first expression repressor in the nucleus of a cell does not overlap with the presence of the second expression repressor in the nucleus of the same cell, wherein the system achieves a decrease in expression of a plurality of genes via the non-overlapping presences of the first and second expression repressors. In some embodiments, the first expression repressor and a second expression repressor may act simultaneously or sequentially.

In some embodiments, the expression repressors of a system each comprise a different targeting moiety (e.g., the first, second, third, or further expression repressors each comprise different targeting moieties from one another). For example, a system may comprise a first expression repressor and a second expression repressor wherein the first expression repressor comprises a first targeting moiety (e.g., a Zn Finger domain, Cas9 domain, or TAL effector domain), and the second expression repressor comprises a second targeting moiety (e.g., a Zn Finger domain, Cas9 domain, or TAL effector domain) different from the first targeting moiety. In some embodiments, different can mean comprising distinct types of targeting moiety, e.g., the first targeting moiety comprises a Cas9 domain, and the second DNA-targeting moiety comprises a Zn finger domain. In other embodiments, different can mean comprising distinct variants of the same type of targeting moiety, e.g., the first targeting moiety comprises a first Cas9 domain (e.g., from a first species) and the second targeting moiety comprises a second Cas9 domain (e.g., from a second species).

In an aspect, systems of the present disclosure may comprise one or more expression repressors and one or more site-specific disrupting agents. In some embodiments, the system comprises one or more expression repressors (e.g., 1, 2, 3, 4, 5, 6, 7, 8, 9, 10, 11, 12, or more (and optionally no more than 15, 14, 13, 12, 11, 10, 9, 8, 7, 6, 5, 4, 3, or 2). In some embodiments, the system comprises one or more site-specific disrupting agents (e.g., 1, 2, 3, 4, 5, 6, 7, 8, 9, 10, 11, 12, or more (and optionally no more than 15, 14, 13, 12, 11, 10, 9, 8, 7, 6, 5, 4, 3, or 2). In some embodiments, a system targets two or more different sequences (e.g., a 1st and 2nd, 3rd, 4th, 5th, 6th, 7th, 8th, 9th, 10th, 11th, 12th, and/or further DNA sequence, and optionally no more than a 20th, 19th, 18th, 17th, 16th, 15th, 14th, 13th, 12th, 11th, 10th, 9th, 8th, 6th, 5th, 4th, 3rd, or 2nd sequence). In some embodiments, the system comprises one or more expression repressors and one or more site-specific disrupting agents, wherein each of the one or more expression repressors and each of the one or more site-specific disrupting agents do not detectably bind, e.g., does not bind, to another expression repressor and/or site-specific disrupting agent. In some embodiments, the system comprises an expression repressor and a site-specific disrupting agent, wherein each of the expression repressor and the site-specific disrupting agent do not detectably bind, e.g., does not bind, to one another.

In some embodiments, the system comprises one or more expression repressors and one or more site-specific disrupting agents, wherein each of the one or more expression repressors and each of the one or more site-specific disrupting agents independently bind a different target. In some embodiments, the system comprises an expression repressor and a site-specific disrupting agent, wherein each of the expression repressor and the site-specific disrupting agent independently bind a different target.

In some embodiments, a system of the present disclosure comprises one or more expression repressors and one or more site-specific disrupting agents, wherein the expression repressors and site-specific disrupting agents are present together in a composition, pharmaceutical composition, or mixture.

In some embodiments, a system of the present disclosure comprises one or more expression repressors and one or more site-specific disrupting agents, wherein the one or more expression repressors and the one or more site-specific disrupting agents are not admixed with at least one other expression repressor and/or site-specific disrupting agent. In some embodiments, a system may comprise an expression repressor and a site-specific disrupting agent, wherein the presence of the expression repressor in the nucleus of a cell does not overlap with the presence of the site-specific disrupting agent in the nucleus of the same cell, wherein the system achieves a decrease in expression of a plurality of genes via the non-overlapping presences of the expression repressor and the site-specific disrupting agent. In some embodiments, the expression repressor and a site-specific disrupting agent may act simultaneously or sequentially.

In some embodiments, the expression repressors and the site-specific disrupting agents of a system each comprise a different targeting moiety (e.g., the first, second, third, or further expression repressors each comprise different targeting moieties from one another and/or a first, second, third, or further site-specific disrupting agents each comprise different targeting moieties from one another). In some embodiments, the one or more expression repressors comprise different targeting moieties from the one or more site-specific disrupting agents. For example, a system may comprise an expression repressor and a site-specific disrupting agent wherein the expression repressor comprises a first targeting moiety (e.g., a Zn Finger domain, Cas9 domain, or TAL effector domain), and the site-specific disrupting agent comprises a second targeting moiety (e.g., a Zn Finger domain, Cas9 domain, or TAL effector domain) different from the first targeting moiety. In some embodiments, different can mean comprising distinct types of targeting moiety, e.g., the first targeting moiety comprises a Cas9 domain, and the second DNA-targeting moiety comprises a Zn finger domain. In other embodiments, different can mean comprising distinct variants of the same type of targeting moiety, e.g., the first targeting moiety comprises a first Cas9 domain (e.g., from a first species) and the second targeting moiety comprises a second Cas9 domain (e.g., from a second species).

In an embodiment, when a system comprises two or more targeting moieties of the same type, e.g., two or more Cas9 or Zn finger domains, the targeting moieties specifically bind two or more different sequences. For example, in a system comprising two or more Cas9 domains, the two or more Cas9 domains may be chosen or altered such that they only appreciably bind the gRNA corresponding to their target sequence (e.g., and do not appreciably bind the gRNA corresponding to the target of another Cas9 domain). In a further example, in a system comprising two or more effector moieties, the two or more effector moieties may be chosen or altered such that they only appreciably bind to their target sequence (e.g., and do not appreciably bind the target sequence of another effector moiety).

In some embodiments, a system comprises three or more site-specific disrupting agents and two or more site-specific disrupting agents comprise the same targeting moiety. For example, a system may comprise three site-specific disrupting agents, wherein the first and second site-specific disrupting agents both comprise a first targeting moiety and the third site-specific disrupting agent comprises a second different targeting moiety. For a further example, a system may comprise four site-specific disrupting agents, wherein the first and second site-specific disrupting agents both comprise a first targeting moiety and the third and fourth site-specific disrupting agents comprises a second different targeting moiety. For a further example, a system may comprise five site-specific disrupting agents, wherein the first and second site-specific disrupting agents both comprise a first targeting moiety, the third and fourth site-specific disrupting agents both comprise a second different targeting moiety, and the fifth site-specific disrupting agent comprises a third different targeting moiety. As described above, different can mean comprising different types of −targeting moieties or comprising distinct variants of the same type of targeting moiety.

In some embodiments, the site-specific disrupting agents of a system each bind to a different DNA sequence (e.g., the first, second, third, or further site-specific disrupting agents each bind DNA sequences that are different from one another). For example, a system may comprise a first site-specific disrupting agent and a second site-specific disrupting agent wherein the first site-specific disrupting agent binds to a first DNA sequence, and the second site-specific disrupting agent binds to a second DNA sequence. In some embodiments involving different DNA sequences, there is at least one position that is not identical between the DNA sequence bound by one site-specific disrupting agent and the DNA sequence bound by another site-specific disrupting agent, or there is at least one position present in the DNA sequence bound by one site-specific disrupting agent that is not present in the DNA sequence bound by another site-specific disrupting agent.

In some embodiments, the first DNA sequence may be situated on a first genomic DNA strand and the second DNA sequence may be situated on a second genomic DNA strand. In some embodiments, the first DNA sequence may be situated on the same genomic DNA strand as the second DNA sequence.

In some embodiments, a system comprises three or more expression repressors and two or more of the expression repressors bind the same DNA sequence. For example, a system may comprise three expression repressors, wherein a first and a second expression repressor both bind a first DNA sequence, and a third expression repressor binds a second different DNA sequence. For a further example, a system may comprise four expression repressors, wherein a first and a second expression repressor both bind a first DNA sequence and a third and a fourth expression repressor both bind a second DNA sequence. For a further example, a system may comprise five expression repressors, wherein a first and a second expression repressor both bind a first DNA sequence, a third and a fourth expression repressor both bind a second DNA sequence, and a fifth expression repressor binds a third DNA sequence. As described above, different can mean that there is at least one position that is not identical between the DNA sequence bound by one expression repressor and the DNA sequence bound by another expression repressor, or that there is at least one position present in the DNA sequence bound by one expression repressor that is not present in the DNA sequence bound by another expression repressor. Similarly, in some embodiments, a system comprises one or more expression repressors and one or more site-specific disrupting agents.

In some embodiments, a system comprises two or more (e.g., two) expression repressors and a plurality (e.g., two) of the expression repressors comprise targeting moieties that bind to different DNA sequences. In such embodiments, a first targeting moiety may bind to a first DNA sequence and a second targeting moiety may bind to a second DNA sequence, wherein the first and the second DNA sequences are different and do not overlap. In some such embodiments, the first DNA sequence is separated from the second DNA sequence by at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs (and optionally, no more than 500, 400, 300, 200, 100, 95, 90, 85, 80, 75, 70, 65, 60, 55, or 50 base pairs). In some such embodiments, the first DNA sequence is separated from the second DNA sequence by no more than 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs (and optionally, no base pairs, e.g., the first and second sequence are directly adjacent one another). In some embodiments, the first DNA sequence is separated from the second DNA sequence by at least 1 kb, 2 kb, 3 kb, 4 kb, or 5 kb.

In some embodiments, a system comprises two or more (e.g., two) site-specific disrupting agents and a plurality (e.g., two) of the site-specific disrupting agents comprise targeting moieties that bind to different DNA sequences. In such embodiments, a first targeting moiety may bind to a first DNA sequence and a second DNA-targeting moiety may bind to a second DNA sequence, wherein the first and the second DNA sequences are different and do not overlap. In some such embodiments, the first DNA sequence is separated from the second DNA sequence by at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs (and optionally, no more than 500, 400, 300, 200, 100, 95, 90, 85, 80, 75, 70, 65, 60, 55, or 50 base pairs). In some such embodiments, the first DNA sequence is separated from the second DNA sequence by no more than 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs (and optionally, no base pairs, e.g., the first and second sequence are directly adjacent one another).

In some embodiments, the expression repressors and/or site-specific disrupting agents of a system each, independently, comprise a different effector moiety (e.g., the first, second, third, or further expression repressors each independently comprise a different effector moiety from one another and/or the first, second, third, or further site-specific disrupting agents each independently comprise a different effector moiety from one another). For example, a system may comprise a first expression repressor and a second expression repressor wherein the first expression repressor comprises a first effector moiety, and the second expression repressor comprises a second effector moiety different from the first effector moiety. Furthermore, a system may comprise an expression repressor and a site-specific disrupting agent wherein the expression repressor comprises a first effector moiety, and the site-specific disrupting agent comprises a second effector moiety different from the first effector moiety. In some embodiments, the different effector moieties comprise distinct types of effector moiety. In other embodiments, the different effector moieties comprise distinct variants of the same type of effector moiety.

In some embodiments, the present disclosure provides an expression repressor system comprising a first expression repressor and a second expression repressor. In some embodiments, the first expression repressor comprises a first targeting moiety. In some embodiments, the first targeting moiety comprises a zinc finger domain. In some embodiments, the first targeting moiety comprises a CRISPR/Cas (e.g., a Cas9 or dCas9) domain. In some embodiments, the first targeting moiety comprises a TAL effector domain. In some embodiments, the first expression repressor comprises a first effector moiety. In some embodiments, the first effector moiety comprises a DNA methyltransferase, e.g., MQ1 or a functional fragment thereof, and/or KRAB, e.g., a KRAB domain. In some embodiments, the second expression repressor comprises a second targeting moiety. In some embodiments, the second targeting moiety comprises a zinc finger domain. In some embodiments, the second expression repressor comprises a second effector moiety. In some embodiments, the second effector moiety comprises a DNA methyltransferase, e.g., MQ1 or a functional fragment thereof, and/or KRAB, e.g., a KRAB domain.

In some embodiments, the expression repressor system is encoded by a first nucleic acid encoding the first expression repressor, e.g., the first targeting moiety and first effector moiety, wherein expression is driven by a first promoter or IRES, and a second nucleic acid encoding the second expression repressor, e.g., the second targeting moiety and second effector moiety, wherein expression is driven by a second promoter or TRES. In some embodiments, the expression repressor system is encoded by a nucleic acid wherein expression is not driven by a promotor or IRES, e.g., an mRNA. In some embodiments, mono-cistronic sequences are used. In some embodiments, the nucleic acid encoding the expression repressor system is a poly-cistronic sequence. In some embodiments, the poly-cistronic sequence is a bi-cistronic sequence. In some embodiments, the poly-cistronic sequence comprises a sequence encoding the first expression repressor and a sequence encoding the second expression repressor. In some embodiments, the poly-cistronic sequence encodes a self-cleavable peptide sequence, e.g., a 2A peptide sequence, e.g., a T2A peptide sequence, a P2A sequence. In some embodiments, the poly-cistronic sequence encodes a T2A peptide sequence and a P2A peptide sequence. In some embodiments, the poly-cistronic sequence encodes a tandem 2A sequence, e.g., a tPT2A sequence. In some embodiments, the bi-cistronic construct further comprises a polyA tail. In some embodiments, upon transcription of the bi-cistronic gene construct, a single mRNA transcript encoding the first expression repressor, and the second expression repressor are produced, which upon translation gets cleaved, e.g., after the glycine residue within the 2A peptide, to yield the first expression repressor and the second expression repressor as two separate proteins. In some embodiments, the first and the second expression repressor are separated by “ribosome-skipping”. In some embodiments the first expression repressor and/or the second expression repressor retains a fragment of the 2A peptide after ribosome skipping. In some embodiments, the expression level of the first and second expression repressor are equal. In some embodiments, the expression level of the first and the second expression repressor are different. In some embodiments, the protein level of the first expression repressor is within 1%, 2%, 5%, or 10% of (greater than or less than) the protein level of the second expression repressor.

In another aspect, the present disclosure provides a system comprising at least one expression repressor as described herein and at least one site-specific disrupting agent (e.g., any site-specific disrupting agent described herein). In some embodiments, the system comprises a first expression repressor and a first site-specific disrupting agent. In some embodiments, the first expression repressor comprises a first targeting moiety. In some embodiments, the first targeting moiety comprises a zinc finger domain. In some embodiments, the first expression repressor comprises a first effector moiety. In some embodiments, the first effector moiety comprises a DNA methyltransferase, e.g., MQ1 or a functional fragment thereof, and/or KRAB, e.g., a KRAB domain. In some embodiments, the site-specific disrupting agent comprises a second targeting moiety, wherein the second targeting moiety targets an anchor sequence of the CXCL locus. In some embodiments, the site-specific disrupting agent comprises a second effector moiety (e.g., a site-specific disrupting agent effector moiety). In some embodiments, the second effector moiety (e.g., a site-specific disrupting agent effector moiety) comprises a DNA methyltransferase, e.g., MQ1 or a functional fragment thereof, and/or KRAB, e.g., a KRAB domain. In some embodiments, the expression repressor effector moiety is the same as the site-specific disrupting agent effector moiety. In some embodiments, the first effector moiety (e.g., the expression repressor effector moiety) is different from the second effector moiety (e.g., the site-specific disrupting agent effector moiety). In some embodiments, the first effector moiety (e.g., the expression repressor effector moiety) and the second effector moiety (e.g., the site-specific disrupting agent moiety) each, independently, comprise methyltransferase activity, e.g., comprise DNA methyltransferase activity.

In some embodiments, the expression repressor system is encoded by a first nucleic acid encoding the first expression repressor, e.g., the first targeting moiety and first effector moiety, wherein expression is driven by a first promoter or IRES, and a second nucleic acid encoding the site-specific disrupting agent, e.g., the second targeting moiety and second effector moiety, wherein expression is driven by a second promoter or IRES. In some embodiments, the expression repressor system is encoded by a nucleic acid wherein expression is not driven by a promotor or IRES, e.g., an mRNA. In some embodiments, mono-cistronic sequences are used. In some embodiments, the nucleic acid encoding the expression repressor system is a poly-cistronic sequence. In some embodiments, the poly-cistronic sequence is a bi-cistronic sequence. In some embodiments, the multi-cistronic sequence comprises a sequence encoding the first expression repressor and a sequence encoding the site-specific disrupting agent. In some embodiments, the poly-cistronic sequence encodes a self-cleavable peptide sequence, e.g., a 2A peptide sequence, e.g., a T2A peptide sequence, a P2A sequence. In some embodiments, the poly-cistronic sequence encodes a T2A peptide sequence and a P2A peptide sequence. In some embodiments, the poly-cistronic sequence encodes a tandem 2A sequence, e.g., a tPT2A sequence. In some embodiments, the bi-cistronic construct further comprises a polyA tail. In some embodiments, upon transcription of the bi-cistronic gene construct, a single mRNA transcript encoding the first expression repressor, and the site-specific disrupting agent are produced, which upon translation gets cleaved, e.g., after the glycine residue within the 2A peptide, to yield the first expression repressor and the site-specific disrupting agent as two separate proteins. In some embodiments, the first expression repressor and the site-specific disrupting agent are separated by “ribosome-skipping”. In some embodiments the first expression repressor and/or the site-specific disrupting agent retains a fragment of the 2A peptide after ribosome skipping. In some embodiments, the expression level of the first expression repressor and the site-specific disrupting agent are equal. In some embodiments, the expression level of the first expression repressor and the site-specific disrupting agent are different. In some embodiments, the protein level of the first expression repressor is within 1%, 2%, 5%, or 10% of (greater than or less than) the protein level of the site-specific disrupting agent.

### Targeting Moieties

Targeting moieties may specifically bind a DNA sequence, e.g., a DNA sequence associated with a target plurality of genes, e.g., a genomic regulatory element or an anchor sequence of an ASMC comprising the target plurality of genes. Any molecule or compound that specifically binds a DNA sequence may be used as a targeting moiety. In some embodiments, a targeting moiety comprises a nucleic acid, e.g., comprising a sequence that is complementary to an enhancer sequence, e.g., an sequence operably linked to the target plurality of genes. In some embodiments, a targeting moiety of a site-specific disrupting agent comprises a nucleic acid, e.g., comprising a sequence that is complementary to an anchor sequence, e.g., an anchor sequence of an ASMC comprising the target plurality of genes. In some embodiments, the nucleic acid is an oligonucleotide that physically/sterically blocks binding of a factor (e.g., a transcription factor, e.g., P65, or a nucleating polypeptide, e.g., CTCF) to a sequence (e.g., an enhancer sequence or an anchor sequence). In some embodiments, the nucleic acid comprises a guide RNA (gRNA), e.g., compatible with a CRISPR/Cas molecule. In some embodiments, a targeting moiety comprises a CRISPR/Cas molecule, a TAL effector molecule, a Zn finger molecule, a tetR domain, a meganuclease, a peptide nucleic acid (PNA) or a nucleic acid.

In some embodiments, the targeting moiety specifically binds to a nucleic acid sequence within an E1 or E2 cRE of the CXCL locus. In some embodiments, the targeting moiety specifically binds to a nucleic acid sequence within the E1 cRE of the CXCL locus. In certain embodiments, the targeting moiety (e.g., an E1-targeting moiety) specifically binds a region within the nucleic acid sequence of SEQ ID NO: 162, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, the targeting moiety specifically binds to a nucleic acid sequence with the E2 cRE of the CXCL locus. In certain embodiments, the targeting moiety (e.g., an E2-targeting moiety) specifically binds a region within the nucleic acid sequence of SEQ ID NO: 163, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

Exemplary sequence of the E11 cRE of the CXCL Locus, SEQ ID NO: 162:

AATGGTTTTCCTGAATTTAGTAGATTTAGGTTTAGATTGGACATG
TCATGTGTGTGAAAGCAGATGTGGTTAATAGGGTAGTGGATAGAA
CAGAATTCTTCCCAGAAAGAGCTTCTATTAGGTCATATTCAAAGG
AACTTTTCTTCCCACGATACCATATTTGCGTTGACACAAAATTTC
ACCTTTTTTCCTTCTTACCCTCTTCCAGAAACACAAATAATTTTT
ATGTGAAGAAAATGTTTACCAAAATTCTAAGGTAAACCTTATATA
GCTTCCTGTATGTCATTTTGTGGCTCCTAATGGCAAAGTTAAGGA
GAGAAGTTTCTGCAATTGTGACTTATCTACAGATAAACAATGCCT
TATTATTATTATTCTTGAGGCATCTGTGAGGGAAAATTCAATAAG
GAAAGGGGATTCAAAGGGAGATTTTAAAAAATCCTTGGGAAATTC
CACACATGGTCAAGGGGCTCATACCCATGACTCCTGAGGCATGTC
AGCCAAAGACATTGCACAGGATTTTATACTGAGTCACCTCTCTGG
AATCTGAGATCTGAATGGAGCCTGGAGAAGGAAGTTGAGTGTCCT
TGGTCACAGTTACCACTTACTTGCTGAGCCAGAAGAATTCTGGAA
GAGTTCCTCTCTTCAGGAGATTTTTTAATGTACCCACCATACGTG
AGGATTTTAGgatccaattcagaccctgttacacagaggtcaccg
tccagaagggagacacaagaaatcacttatgatacagcacattgg
actaaagcagtgattcttaagttctggtgcctggacaagcatcat
cagcatcacctgtgggctagttggaaaagacaattttggaggcct
ccttttcccttctgaaacagacatggtgggagtggagtccagtga
gctgtgtttcagcaacccttccaggggattctgacatactgtaaa
gtctgagaacccactgggctagaaggaagcctcctggcatgagga
gagagttgttctggaggccttaaataacatgggagtaggttgagg
acaaggttgagaaggtggtggTGCCTAGACCTTTTAAAGGATTAA
TAAGATTCTTGAGaaaaaaaaaaGCCAGCAGAGGGAAGAAGAATG
GAAAAGCTAAAGCTGAGGGTGACAGGGAGGAAGATAGACCCAGAG
AAGAGTCTGGAGAGGCCAGAAGGGGCCAAGTGGGGAGAGAATTTT
ATCAGGGCTGGGAGTATAAACTGTATTATGAAGACCATGGCACTG
GGATAAAGGCTGAAGGCTAAAGTCTGGGAAAGACACACTCAGATT
TGTAGAAAAGCTATAAAATTTCAGATTTTAGAAAGCTCACTTGGC
AGATGTGAGTGAAAAGTGGAGGGGAGGAAGCTCAAAGTTGAAGCA
GTTAGGAGATTTCTATGGTGATCTCGGGCCAGGGAAAGCAGGACT
CACCTAGCACAGGGGGCTAGGAAGGGGAAGGGTCTCAAACAGGAG
AACAGCTGAGAAAAGGGAGTTGGTAGAACCTCATGTTATAATACA
TCACAGAAGCCAAAAATCATGAGGTAGTATACTGACTGGCAGCAA
ATGCCTATGAGATTGGAAAGTGAGAT

Exemplary sequence of the E2 cRE of the CXCL Locus, SEQ ID NO: 163:

GTTAGGTTTTATTTCACCAAGTTTATAAAGTAAACCACATATCGT
TTTCTCTTTTGTAGATGCTGAAAGCAAAGTTCATGTGGGAAATGT
TTGGCAATAGCTGATTTATCCTCAGGGTAACAATATTCTATAACT
CCTTTGATCTTGAGGCCTCTGTGATGGAAATGCTTGGAGAAAGGG
ATTTTAAAGGGAGATTCTGAAGTCCTTGGGAAAGTCCACAAGTGG
ACGGGGCTTCATAGCCATGACAACAAATGACATTGTCTAGGAAAC
AGTGAGTCATGGCATGCTGAGCTTAGAATGGAGCCAACAGAAGGA
ACCTGGCCTCGGACACAGAATCTTTTGGCTGCTGACCCAGAATGA
CTGTGAAAGACTAACACTGTTTAGCAGATTTTTCTTGAGTGTTTA
CTATGTGTGAGGTTCCTGGGATTCAGATTCAGCTACTATTGTTAA
GAGGAAATCAACCAGGAAGTCAGTTAAGAAAAGGTACAGTGGGTT
TTCAGGCTGCAGGGTACAGAAATGTTCCCAGGCCTGGAGAACAAA
CCTTCAGATCTTAATCTGTACAGGGAGGTGGAGGGTGAAAGAATG
ATCTTTCAGGAAGCGTTCAAGTAGGGCTGCTGCTTGGATTGAATT
TTAAAGAATGCATAGGTTATATGCAGGATCTATATATAGATCAAT
AGCTTCCCTGAGCACATGTTCAAAGGTTCAAACATTTGGGGTCAT
TTCTTTGCAAGAAGAGTCACTCAGTGGCCTGAAAGTCCATGCAGC
AACTTCCCTCATGAGAGCTGCTTCCGCAGCAGGCCCAGGGTTTCT
AAAGGAGAGAGCACACAGATGTAAACACTCTGTGGTTCTGAGGAC
TGTCACCTCTTCTTTTCACCCATCACTTTTGTCTTAAGAACTCTA
TGCTCAACCCTAATTCTCAGTCTCTATATCAATTCCCACCAAACA
GATGCAAAGTCCTGTCC

In some embodiments, the targeting moiety specifically binds to a nucleic acid sequence within the IL8 promoter. In some embodiments, the target site (e.g., target site within the IL8 promoter) is within genomic coordinates chr4:74606112-74606462 (hg19). In some embodiments, the target site (e.g., target site within the IL8 promoter) is located within 1 kb from chr:74606112-74606462 (e.g., chr4:74606112-74606662, chr4:74606112-74606862, chr4:74606112-74607062, chr4:74606112-74607262, chr4:74606112-74607462, chr4:74605912-74606462, chr4:74605712-74606462, chr4:74605512-74606462, chr4:74605312-74606462, chr4:74605112-74606462, chr4:74605912-74606662, chr4:74605912-74606862, chr4:74605912-74607062, chr4:74605912-74607262, chr4:74605912-74607462, chr4:74605712-74606662, chr4:74605712-74606862, chr4:74605712-74607062, chr4:74605712-74607262, chr4:74605712-74607462, chr4:74605512-74606662, chr4:74605512-74606862, chr4:74605512-74607062, chr4:74605512-74607262, chr4:74605512-74607462, chr4:74605312-74606662, chr4:74605312-74606862, chr4:74605312-74607062, chr4:74605312-74607262, chr4:74605312-74607462, chr4:74605112-74606662, chr4:74605112-74606862, chr4:74605112-74607062, chr4:74605112-74607262, or chr4:74605112-74607462).

In some embodiments, the target site (e.g., target site within the IL8 promoter) is located 500 bp upstream from the transcription start site. In certain embodiments, the target site (e.g., target site within the IL8 promoter) is located at chr4:74605723-74606223. In some embodiments, the target site (e.g., target site within the IL8 promoter) is located at chr4:74605723-74606426, chr4:74605723-74606626, chr4:74605723-74606826, chr4:74605723-74607026, chr4:74605723-74607226, chr4:74605523-74606226, chr4:74605323-74606226, chr4:74605123-74606226, chr4:74604923-74606226, chr4:74604723-74606226, chr4:74605523-74606426, chr4:74605523-74606626, chr4:74605523-74606826, chr4:74605523-74607026, chr4:74605523-74607226, chr4:74605323-74606426, chr4:74605323-74606626, chr4:74605323-74606826, chr4:74605323-74607026, chr4:74605323-74607226, chr4:74605123-74606426, chr4:74605123-74606626, chr4:74605123-74606826, chr4:74605123-74607026, chr4:74605123-74607226, chr4:74604923-74606426, chr4:74604923-74606626, chr4:74604923-74606826, chr4:74604923-74607026, chr4:74604923-74607226, chr4:74604723-74606426, chr4:74604723-74606626, chr4:74604723-74606826, chr4:74604723-74607026, or chr4:74604723-74607226.

In some embodiments, the target site (e.g., target site within the IL8 promoter) is located 1000 bp upstream from the transcription start site. In certain embodiments, the target site (e.g., target site within the IL8 promoter) is located at chr4:74605223-74606223. In some embodiments, the target site (e.g., target site within the IL8 promoter) is located at chr4:74605226-74606426, chr4:74605226-74606626, chr4:74605226-74606826, chr4:74605226-74607026, chr4:74605226-74607226, chr4:74605026-74606226, chr4:74604826-74606226, chr4:74604626-74606226, chr4:74604426-74606226, chr4:74604226-74606226, chr4:74605026-74606426, chr4:74605026-74606626, chr4:74605026-74606826, chr4:74605026-74607026, chr4:74605026-74607226, chr4:74604826-74606426, chr4:74604826-74606626, chr4:74604826-74606826, chr4:74604826-74607026, chr4:74604826-74607226, chr4:74604626-74606426, chr4:74604626-74606626, chr4:74604626-74606826, chr4:74604626-74607026, chr4:74604626-74607226, chr4:74604426-74606426, chr4:74604426-74606626, chr4:74604426-74606826, chr4:74604426-74607026, chr4:74604426-74607226, chr4:74604226-74606426, chr4:74604226-74606626, chr4:74604226-74606826, chr4:74604226-74607026, or chr4:74604226-74607226.

In some embodiments, a targeting moiety binds to its target sequence with a K_{D} of less than or equal to 500, 450, 400, 350, 300, 250, 200, 150, 100, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.002, or 0.001 nM (and optionally, a K_{D} of at least 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.002, or 0.001 nM). In some embodiments, a targeting moiety binds to its target sequence with a K_{D} of 0.001 nM to 500 nM, e.g., 0.1 nM to 5 nM, e.g., about 0.5 nM. In some embodiments, a targeting moiety binds to a non-target sequence with a K_{D} of at least 500, 600, 700, 800, 900, 1000, 2000, 5000, 10,000, or 100,000 nM (and optionally, does not appreciably bind to a non-target sequence). In some embodiments, a targeting moiety does not bind to a non-target sequence.

In some embodiments, a targeting moiety of an expression repressor or a site-specific disrupting agent comprises a nucleic acid comprising a sequence complementary to a sequence selected from Table 8 or 8A or a sequence having at least 70, 75, 80, 85, 90, 91, 92, 93, 94, 95, 96, 97, 98, or 99% identity thereto, or differing at no more than 1, 2, 3, 4, or 5 positions relative thereto. In some embodiments, a targeting moiety of an expression repressor or a site-specific disrupting agent comprises a nucleic acid comprising a sequence selected from Table 8 or 8A, or a sequence having at least 70, 75, 80, 85, 90, 91, 92, 93, 94, 95, 96, 97, 98, or 99% identity thereto, or differing at no more than 1, 2, 3, 4, or 5 positions relative thereto. In some embodiments, the targeting moiety of an expression repressor or a site-specific disrupting agent binds to a target site having a sequence of Table 8 or 8A. It is understood that, in some embodiments, the targeting moiety comprises an RNA sequence in which each position indicated as a T in Table 8 or 8A is occupied by a U.

TABLE 8
Exemplary sequence or target
sequences of gRNA spacers

| | | SEQ ID |
|---|---|---|
| Name | Sequence | NO. |
| GD-31493 | TTATTCTTGAGGCATCTGTG | 90 |
| GD-31494 | AAATTCCACACATGGTCAAG | 91 |
| GD-31495 | GTCAGCCAAAGACATTGCAC | 92 |
| GD-31496 | CTGTGTAACAGGGTCTGAAT | 93 |
| GD-31497 | TATAGAATATTGTTACCCTG | 94 |
| GD-31498 | TCCTTGGGAAAGTCCACAAG | 95 |
| GD-31499 | GACAACAAATGACATTGTCT | 96 |
| GD-31500 | GCCAAAAGATTCTGTGTCCG | 97 |
| GD-31501 | TCAGTTAAGAAAAGGTACAG | 98 |
| GD-31502 | TTCAGATCTTAATCTGTACA | 99 |
| GD-31503 | ACTGATGGCCCATCCCCTCA | 100 |

TABLE 8A
Exemplary sequence or target sequences
of gRNA spacers, e.g., for use
in a murine model

| | | SEQ ID |
|---|---|---|
| Name | Sequence | NO. |
| GD-33448 | GTTAGCCCTAAATCGACCCT | 341 |
| GD-33449 | GGTGTGCAGTTAGCACAACT | 342 |
| GD-33450 | GAATTGCAGACATCAGTCTG | 343 |
| GD-33451 | AGGGGTCACCAAGAAGAGTG | 344 |
| GD-33452 | GGTTTCTCATTAGCAATCAG | 345 |
| GD-33453 | TCCTTGGGAAATTCCACTTG | 346 |
| GD-33454 | GTCATAACCCAGGAGTCACG | 347 |
| GD-33455 | GTTTCTGGGCTAGAGAACTC | 348 |
| GD-33456 | AATGGAACAAGTCAGCATGA | 349 |
| GD-33457 | CTGGCAAGGGTGAGACAGAG | 350 |
| GD-33458 | CATGGTAAACAACAGTACAC | 351 |
| GD-33459 | GTTGTGGATATATCCAGGCA | 352 |
| GD-33460 | TACGCATGAATGAGATCACT | 353 |
| GD-33461 | TTGTTTTTAATGCTTGCATG | 354 |
| GD-33462 | TCATTCCGTGACATCCAGGA | 355 |
| GD-33463 | CCTCCGTGACTAAGCCAAGG | 356 |
| GD-33464 | CCCCTACTCTCAGCCTGCAT | 357 |
| GD-33465 | ACTCCCCCTCTGTTTTACAC | 358 |
| GD-33466 | CTGAGTTTGTGTGGAGTATG | 359 |
| GD-33467 | TTTAGACTACTGTCTGACTG | 360 |
| GD-33468 | AGCTGTGGCAGTGATGCGAG | 361 |
| GD-33469 | AACAGGAAATATTCCTCCCA | 362 |
| GD-33470 | AGGATCTTCTGGAAGTGTCA | 363 |

In some embodiments, a targeting moiety comprises a nucleic acid comprising a sequence selected from Table 9 or 9A, or a sequence having at least 70, 75, 80, 85, 90, 91, 92 93, 94, 95 0.96, 97, 98, or 99%˜identity thereto, or differing at no more than 1, 2, 3, 4, or 5 positions relative thereto. In some embodiments, a targeting moiety comprises a nucleic acid comprising a spacer sequence within a sequence of Table 9 or 9A, or a sequence having at least 70, 75, 80, 85, 90, 91, 92, 93, 94, 95, 96, 97, 98, or 99% identity thereto, or differing at no more than 1, 2, 3, 4, or 5 positions relative thereto. It is understood that, in some embodiments, the targeting moiety comprises an RNA sequence in which each position indicated as a T in Table 9 or 9A is occupied by a U.

TABLE 9
Exemplary guide sequences

| | | SEQ ID | Strand | Genomic |
|---|---|---|---|---|
| Guide | Sequence | NO | | Coordinate |
| GD-31493 | TTATTCTTGAGGCATCTGTGG | 101 | + | GRCh37: |
| | TTTTAGAGCTAGAAATAGCAA | | | chr4: 74591768- |
| | GTTAAAATAAGGCTAGTCCGT | | | 74591790 |
| | TATCAACTTGAAAAAGTGGCA | | | |
| | CCGAGTCGGTGCTTTT | | | |
| GD-31494 | AAATTCCACACATGGTCAAGG | 102 | + | GRCh37: |
| | TTTTAGAGCTAGAAATAGCAA | | | chr4: 74591844- |
| | GTTAAAATAAGGCTAGTCCGT | | | 74591866 |
| | TATCAACTTGAAAAAGTGGCA | | | |
| | CCGAGTCGGTGCTTTT | | | |
| GD-31495 | GTCAGCCAAAGACATTGCACG | 103 | + | GRCh37: |
| | TTTTAGAGCTAGAAATAGCAA | | | chr4: 74591892- |
| | GTTAAAATAAGGCTAGTCCGT | | | 74591914 |
| | TATCAACTTGAAAAAGTGGCA | | | |
| | CCGAGTCGGTGCTTTT | | | |
| GD-31496 | ctgtgtaacagggtctgaatG | 104 | − | GRCh37: |
| | TTTTAGAGCTAGAAATAGCAA | | | chr4: 74592088- |
| | GTTAAAATAAGGCTAGTCCGT | | | 74592110 |
| | TATCAACTTGAAAAAGTGGCA | | | |
| | CCGAGTCGGTGCTTTT | | | |
| GD-31497 | TATAGAATATTGTTACCCTGG | 105 | − | GRCh37: |
| | TTTTAGAGCTAGAAATAGCAA | | | chr4: 74982748- |
| | GTTAAAATAAGGCTAGTCCGT | | | 74982770 |
| | TATCAACTTGAAAAAGTGGCA | | | |
| | CCGAGTCGGTGCTTTT | | | |
| GD-31498 | TCCTTGGGAAAGTCCACAAGG | 106 | + | GRCh37: |
| | TTTTAGAGCTAGAAATAGCAA | | | chr4: 74982841- |
| | GTTAAAATAAGGCTAGTCCGT | | | 74982863 |
| | TATCAACTTGAAAAAGTGGCA | | | |
| | CCGAGTCGGTGCTTTT | | | |
| GD-31499 | GACAACAAATGACATTGTCTG | 107 | + | GRCh37: |
| | TTTTAGAGCTAGAAATAGCAA | | | chr4: 74982882- |
| | GTTAAAATAAGGCTAGTCCGT | | | 74982904 |
| | TATCAACTTGAAAAAGTGGCA | | | |
| | CCGAGTCGGTGCTTTT | | | |
| GD-31500 | GCCAAAAGATTCTGTGTCCGG | 108 | − | GRCh37: |
| | TTTTAGAGCTAGAAATAGCAA | | | chr4: 74982960- |
| | GTTAAAATAAGGCTAGTCCGT | | | 74982982 |
| | TATCAACTTGAAAAAGTGGCA | | | |
| | CCGAGTCGGTGCTTTT | | | |
| GD-31501 | TCAGTTAAGAAAAGGTACAG | 109 | + | GRCh37: |
| | GTTTTAGAGCTAGAAATAGCA | | | chr4: 74983108- |
| | AGTTAAAATAAGGCTAGTCCG | | | 74983130 |
| | TTATCAACTTGAAAAAGTGGC | | | |
| | ACCGAGTCGGTGCTTTT | | | |
| GD-31502 | TTCAGATCTTAATCTGTACAG | 110 | + | GRCh37: |
| | TTTTAGAGCTAGAAATAGCAA | | | chr4: 74983181- |
| | GTTAAAATAAGGCTAGTCCGT | | | 74983203 |
| | TATCAACTTGAAAAAGTGGCA | | | |
| | CCGAGTCGGTGCTTTT | | | |
| GD-31503 | ACTGATGGCCCATCCCCTCAG | 111 | − | GRCh37: |
| | TTTTAGAGCTAGAAATAGCAA | | | chr4: 74606162- |
| | GTTAAAATAAGGCTAGTCCGT | | | 74606184 |
| | TATCAACTTGAAAAAGTGGCA | | | |
| | CCGAGTCGGTGCTTTT | | | |

TABLE 9A
Exemplary guide sequences, e.g., for use in a murine model

| | | SEQ ID | | Genomic |
|---|---|---|---|---|
| Guide | Sequence | NO | Strand | Coordinate |
| GD-33448 | GTTAGCCCTAAATCGACCCTGTTT | 364 | - | GRCm38: |
| | TAGAGCTAGAAATAGCAAGTTAAA | | | chr5: 90702981- |
| | ATAAGGCTAGTCCGTTATCAACTT | | | 90703003 |
| | GAAAAAGTGGCACCGAGTCGGTGC | | | |
| | TTTT | | | |
| GD-33449 | GGTGTGCAGTTAGCACAACTGTTT | 365 | + | GRCm38: |
| | TAGAGCTAGAAATAGCAAGTTAAA | | | chr5: 90703037- |
| | ATAAGGCTAGTCCGTTATCAACTT | | | 90703059 |
| | GAAAAAGTGGCACCGAGTCGGTGC | | | |
| | TTTT | | | |
| GD-33450 | GAATTGCAGACATCAGTCTGGTTT | 366 | − | GRCm38: |
| | TAGAGCTAGAAATAGCAAGTTAAA | | | chr5: 90703085- |
| | ATAAGGCTAGTCCGTTATCAACTT | | | 90703107 |
| | GAAAAAGTGGCACCGAGTCGGTGC | | | |
| | TTTT | | | |
| GD-33451 | AGGGGTCACCAAGAAGAGTGGTTT | 367 | − | GRCm38: |
| | TAGAGCTAGAAATAGCAAGTTAAA | | | chr5: 90706313- |
| | ATAAGGCTAGTCCGTTATCAACTT | | | 90706335 |
| | GAAAAAGTGGCACCGAGTCGGTGC | | | |
| | TTTT | | | |
| GD-33452 | GGTTTCTCATTAGCAATCAGGTTT | 368 | − | GRCm38: |
| | TAGAGCTAGAAATAGCAAGTTAAA | | | chr5: 90706331- |
| | ATAAGGCTAGTCCGTTATCAACTT | | | 90706353 |
| | GAAAAAGTGGCACCGAGTCGGTGC | | | |
| | TTTT | | | |
| GD-33453 | TCCTTGGGAAATTCCACTTGGTTT | 369 | + | GRCm38: |
| | TAGAGCTAGAAATAGCAAGTTAAA | | | chr5: 90706470- |
| | ATAAGGCTAGTCCGTTATCAACTT | | | 90706492 |
| | GAAAAAGTGGCACCGAGTCGGTGC | | | |
| | TTTT | | | |
| GD-33454 | GTCATAACCCAGGAGTCACGGTTT | 370 | − | GRCm38: |
| | TAGAGCTAGAAATAGCAAGTTAAA | | | chr5: 90706505- |
| | ATAAGGCTAGTCCGTTATCAACTT | | | 90706527 |
| | GAAAAAGTGGCACCGAGTCGGTGC | | | |
| | TTTT | | | |
| GD-33455 | GTTTCTGGGCTAGAGAACTCGTTT | 371 | + | GRCm38: |
| | TAGAGCTAGAAATAGCAAGTTAAA | | | chr5: 90706620- |
| | ATAAGGCTAGTCCGTTATCAACTT | | | 90706642 |
| | GAAAAAGTGGCACCGAGTCGGTGC | | | |
| | TTTT | | | |
| GD-33456 | AATGGAACAAGTCAGCATGAGTTT | 372 | + | GRCm38: |
| | TAGAGCTAGAAATAGCAAGTTAAA | | | chr5: 90717935- |
| | ATAAGGCTAGTCCGTTATCAACTT | | | 90717957 |
| | GAAAAAGTGGCACCGAGTCGGTGC | | | |
| | TTTT | | | |
| GD-33457 | ctggcaagggtgagacagagGTTT | 373 | + | GRCm38: |
| | TAGAGCTAGAAATAGCAAGTTAAA | | | chr5: 90718084- |
| | ATAAGGCTAGTCCGTTATCAACTT | | | 90718106 |
| | GAAAAAGTGGCACCGAGTCGGTGC | | | |
| | TTTT | | | |
| GD-33458 | catggtaaacaacagtacacGTTT | 374 | − | GRCm38: |
| | TAGAGCTAGAAATAGCAAGTTAAA | | | chr5: 90718111- |
| | ATAAGGCTAGTCCGTTATCAACTT | | | 90718133 |
| | GAAAAAGTGGCACCGAGTCGGTGC | | | |
| | TTTT | | | |
| GD-33459 | gttgtggatatatccaggcaGTTT | 375 | − | GRCm38: |
| | TAGAGCTAGAAATAGCAAGTTAAA | | | chr5: 90718179- |
| | ATAAGGCTAGTCCGTTATCAACTT | | | 90718201 |
| | GAAAAAGTGGCACCGAGTCGGTGC | | | |
| | TTTT | | | |
| GD-33460 | TACGCATGAATGAGATCACTGTTT | 376 | + | GRCm38: |
| | TAGAGCTAGAAATAGCAAGTTAAA | | | chr5: 90764062- |
| | ATAAGGCTAGTCCGTTATCAACTT | | | 90764084 |
| | GAAAAAGTGGCACCGAGTCGGTGC | | | |
| | TTTT | | | |
| GD-33461 | TTGTTTTTAATGCTTGCATGGTTT | 377 | + | GRCm38: |
| | TAGAGCTAGAAATAGCAAGTTAAA | | | chr5: 90764268- |
| | ATAAGGCTAGTCCGTTATCAACTT | | | 90764290 |
| | GAAAAAGTGGCACCGAGTCGGTGC | | | |
| | TTTT | | | |
| GD-33462 | TCATTCCGTGACATCCAGGAGTTT | 378 | − | GRCm38: |
| | TAGAGCTAGAAATAGCAAGTTAAA | | | chr5: 90764336- |
| | ATAAGGCTAGTCCGTTATCAACTT | | | 90764358 |
| | GAAAAAGTGGCACCGAGTCGGTGC | | | |
| | TTTT | | | |
| GD-33463 | CCTCCGTGACTAAGCCAAGGGTTT | 379 | + | GRCm38: |
| | TAGAGCTAGAAATAGCAAGTTAAA | | | chr5: 90764401- |
| | ATAAGGCTAGTCCGTTATCAACTT | | | 90764423 |
| | GAAAAAGTGGCACCGAGTCGGTGC | | | |
| | TTTT | | | |
| GD-33464 | CCCCTACTCTCAGCCTGCATGTTT | 380 | − | GRCm38: |
| | TAGAGCTAGAAATAGCAAGTTAAA | | | chr5: 90832483- |
| | ATAAGGCTAGTCCGTTATCAACTT | | | 90832505 |
| | GAAAAAGTGGCACCGAGTCGGTGC | | | |
| | TTTT | | | |
| GD-33465 | ACTCCCCCTCTGTTTTACACGTTT | 381 | + | GRCm38: |
| | TAGAGCTAGAAATAGCAAGTTAAA | | | chr5: 90832526- |
| | ATAAGGCTAGTCCGTTATCAACTT | | | 90832548 |
| | GAAAAAGTGGCACCGAGTCGGTGC | | | |
| | TTTT | | | |
| GD-33466 | CTGAGTTTGTGTGGAGTATGGTTT | 382 | + | GRCm38: |
| | TAGAGCTAGAAATAGCAAGTTAAA | | | chr5: 90832598- |
| | ATAAGGCTAGTCCGTTATCAACTT | | | 90832620 |
| | GAAAAAGTGGCACCGAGTCGGTGC | | | |
| | TTTT | | | |
| GD-33467 | tttagactactgtctgactgGITT | 383 | − | GRCm38: |
| | TAGAGCTAGAAATAGCAAGTTAAA | | | chr5: 90930741- |
| | ATAAGGCTAGTCCGTTATCAACTT | | | 90930763 |
| | GAAAAAGTGGCACCGAGTCGGTGC | | | |
| | TTTT | | | |
| GD-33468 | agctgtggcagtgatgcgagGTTT | 384 | − | GRCm38: |
| | TAGAGCTAGAAATAGCAAGTTAAA | | | chr5: 90930773- |
| | ATAAGGCTAGTCCGTTATCAACTT | | | 90930795 |
| | GAAAAAGTGGCACCGAGTCGGTGC | | | |
| | TTTT | | | |
| GD-33469 | aacaggaaatattcctcccaGTTT | 385 | − | GRCm38: |
| | TAGAGCTAGAAATAGCAAGTTAAA | | | chr5: 90930799- |
| | ATAAGGCTAGTCCGTTATCAACTT | | | 90930821 |
| | GAAAAAGTGGCACCGAGTCGGTGC | | | |
| | TTTT | | | |
| GD-33470 | AGGATCTTCTGGAAGTGTCAGTTT | 386 | + | GRCm38: |
| | TAGAGCTAGAAATAGCAAGTTAAA | | | chr5: 90930887- |
| | ATAAGGCTAGTCCGTTATCAACTT | | | 90930909 |
| | GAAAAAGTGGCACCGAGTCGGTGC | | | |
| | TTTT | | | |

In some embodiments, a targeting moiety comprises a nucleic acid comprising a sequence that is complementary to at least a portion of the sequence of a cRE (e.g., an E11 cRE), or having no more than 1, 2, 3, 4, 5, 6, 7, 8, 9, or 10 positions of non-complementarity thereto.

In some embodiments, a targeting moiety comprises a nucleic acid comprising a sequence that is complementary to at least a portion of the sequence of anon-human cRE (e.g., a non-human E11 cRE) homologous to a human cRE (e.g., a mouse cRE), or having no more than 1, 2, 3, 4, 5, 6, 7, 8, 9, or 10 positions of non-complementarity thereto.

In some embodiments, a targeting moiety comprises a nucleic acid comprising a sequence that at least partially overlaps with a region having genomic coordinates GRCh37: chr4:74591768-74591790, GRCh37: chr4:74591844-74591866, GRCh37: chr4:74591892-74591914, GRCh37: chr4:74592088-74592110, GRCh37: chr4:74982748-74982770, GRCh37: chr4:74982841-74982863, GRCh37: chr4:74982882-74982904, GRCh37: chr4:74982960-74982982, GRCh37: chr4:74983108-74983130, GRCh37: chr4:74983181-74983203, or GRCh37: chr4:74606162-74606184.

In some embodiments, a targeting moiety binds to a sequence at genomic position GRCh37: chr4:74591768-74591790, GRCh37: chr4:74591844-74591866, GRCh37: chr4:74591892-74591914, GRCh37: chr4:74592088-74592110, GRCh37: chr4:74982748-74982770, GRCh37: chr4:74982841-74982863, GRCh37: chr4:74982882-74982904, GRCh37: chr4:74982960-74982982, GRCh37: chr4:74983108-74983130, GRCh37: chr4:74983181-74983203, or GRCh37: chr4:74606162-74606184.

In some embodiments, a targeting moiety binds to a cRE (e.g., an E1 cRE) or to a site proximal to a cRE (e.g., an E1 cRE), e.g., a cRE operably linked to a target plurality of genes.

Site-specific disrupting agent gRNA In some embodiments, a targeting moiety or a site-specific disrupting agent comprises a nucleic acid comprising a sequence selected from Table 7 or a sequence having at least 70, 75, 80, 85, 90, 91, 92, 93, 94, 95, 96, 97, 98, or 99% identity thereto, or differing at no more than 1, 2, 3, 4, or 5 positions relative thereto. It is understood that, in some embodiments, the targeting moiety comprises an RNA sequence in which each position indicated as a T in Table 7 is occupied by a U.

TABLE 7
Exemplary gRNA spacer sequences

| Name | Sequence | SEQ ID NO. |
|---|---|---|
| GD-28481 | AGCCCCACCTTGTGGTCAGA | 21 |
| GD-28482 | AGTGCTGCCTTCTGACCACA | 22 |
| GD-28483 | GCTGCCTTCTGACCACAAGG | 23 |
| GD-28484 | CCAGTATAAGCCCCACCTTG | 24 |
| GD-28485 | CTGCCTGTCCCATAAGGAGG | 25 |
| GD-28486 | GCACTGCCTGTCCCATAAGG | 26 |
| GD-28487 | GGTCCTCCTCCTTATGGGAC | 27 |
| GD-28488 | GCCTTGTTTTCGGCTCTAGA | 28 |
| GD-28489 | GCCATCTAGAGCCGAAAACA | 29 |
| GD-29251 | CCAATGAAGATGAAACTGGG | 30 |
| GD-29252 | AACGTGCTTGCCTAAGATTC | 31 |
| GD-29253 | AGCCCTTAATCATATCTAGT | 32 |
| GD-29254 | CAGAGCTTAAGACCTGTACT | 33 |
| GD-29255 | GCCCACCTTGACCTTCACAA | 34 |

In some embodiments, a targeting moiety comprises a nucleic acid comprising a sequence selected from Table 6 or a sequence having at least 70, 75, 80, 85, 90, 91, 92, 93, 94, 95, 96, 97, 98, or 99% identity thereto, or differing at no more than 1, 2, 3, 4, or 5 positions relative thereto. It is understood that, in some embodiments, the targeting moiety comprises an RNA sequence in which each position indicated as a T in Table 6 is occupied by a U.

TABLE 6
Exemplary guide sequence

| Guide | Sequence | Strand | Genomic Coordinate |
|---|---|---|---|
| GD-30178 | GTTACTGCGTAATTACCAGG | + | GRCh37: chr4: 74528428- |
| | (SEQ ID NO: 35) | | 74528450 |
| GD-30179 | TATTACATCCTACCTATAAG | + | GRCh37: chr4: 74528567- |
| | (SEQ ID NO: 36) | | 74528589 |
| GD-30180 | TGGGCTCTGGACTTAGATCG | + | GRCh37: chr4: 74528609- |
| | (SEQ ID NO: 37) | | 74528631 |
| GD-30181 | TAAGTGGGCTATGTATACAC | + | GRCh37: chr4: 74789132- |
| | (SEQ ID NO: 38) | | 74789154 |
| GD-30182 | TTTCTAAGTCTGTCACAAGG | − | GRCh37: chr4: 74789250- |
| | (SEQ ID NO: 39) | | 74789272 |
| GD-30183 | AAAGTAATATGATCTAGGAA | − | GRCh37: chr4: 74789312- |
| | (SEQ ID NO: 40) | | 74789334 |
| GD-30184 | GTTCGAGCGGCTGTGCGAGG | + | GRCh37: chr4: 74964853- |
| | (SEQ ID NO: 41) | | 74964875 |
| GD-30185 | GCTCTGTGGCTCTCCGAGAA | + | GRCh37: chr4: 74964906- |
| | (SEQ ID NO: 42) | | 74964928 |
| GD-30186 | GTGTGTGTGTTTCAACGTAG | + | GRCh37: chr4: 74965538- |
| | (SEQ ID NO: 43) | | 74965560 |
| GD-30187 | GGAAGTCACTGGGAGCTGCG | + | GRCh37: chr4: 74965737- |
| | (SEQ ID NO: 44) | | 74965759 |
| GD-30200 | GGCCACGGGTGTGTTCCCAG | − | GRCh37: chr4: 75000031- |
| | (SEQ ID NO: 45) | | 75000053 |
| GD-30201 | ATGGCCATTTGCAAAAGTCA | + | GRCh37: chr4: 75000115- |
| | (SEQ ID NO: 46) | | 75000137 |
| GD-30202 | CCAAACTAGACAGATAAAGC | + | GRCh37: chr4: 75000231- |
| | (SEQ ID NO: 47) | | 75000253 |
| GD-30203 | CCAGCATGACTCTAGCATGC | − | GRCh37: chr4: 74975146- |
| | (SEQ ID NO: 48) | | 74975168 |
| GD-30204 | TGGCCAAGGTCTGATATGCA | + | GRCh37: chr4: 74975369- |
| | (SEQ ID NO: 49) | | 74975391 |
| GD-30205 | TCATGAGTCCCAGAACATGT | − | GRCh37: chr4: 74976318- |
| | (SEQ ID NO: 50) | | 74976340 |
| GD-30206 | GCGAAAGAAGTAGTAGCTAA | − | GRCh37: chr4: 74570348- |
| | (SEQ ID NO: 51) | | 74570370 |
| GD-30207 | GACTAAGACTGGCAAATCTG | − | GRCh37: chr4: 74570503- |
| | (SEQ ID NO: 52) | | 74570525 |
| GD-30208 | GACTAAGAGGAGCCGACATG | + | GRCh37: chr4: 74570526- |
| | (SEQ ID NO: 53) | | 74570548 |
| GD-30238 | GAAAAACGGGTGTTGTGACG | + | GRCm38: chr5: 90661492- |
| | (SEQ ID NO: 54) | | 90661514 |
| GD-30239 | TTTGTGAACTAAGGATTCTG | − | GRCm38: chr5: 90661646- |
| | (SEQ ID NO: 55) | | 90661668 |
| GD-30240 | GTCCGTGTAGAGTTACCATG | + | GRCm38: chr5: 90661744- |
| | (SEQ ID NO: 56) | | 90661766 |
| GD-30241 | GATGTATTCACAAGAGGACT | + | GRCm38: chr5: 90785610- |
| | (SEQ ID NO: 57) | | 90785632 |
| GD-30242 | AATTACTACCTCATAGCTAG | − | GRCm38: chr5: 90909047- |
| | (SEQ ID NO: 58) | | 90909069 |
| GD-30592 | GAAGGTAGAAATCCGCCACT | | GRCm38: chr5: 90785724- |
| | (SEQ ID NO: 59) | | 90785746 |
| GD-30593 | GAAACGCCGAGGTAACTCAT | | GRCm38: chr5: 90788137- |
| | (SEQ ID NO: 60) | | 90788159 |
| GD-30594 | CAACTAAAATTTCTAGCCCT | | GRCm38: chr5: 90908926- |
| | (SEQ ID NO: 61) | | 90908948 |
| GD-28044 | GACTCCAGTCTTTCTAGAAGA | | GRCm38: chr6: 113076028- |
| | (SEQ ID NO: 62) | | 113076047 |

In some embodiments, a targeting moiety comprises a nucleic acid comprising a sequence that is complementary to the sequence of an anchor sequence, e.g., of an ASMC comprising the target plurality of genes, or having no more than 1, 2, 3, 4, 5, 6, 7, 8, 9, or 10 positions of non-complementarity thereto.

In some embodiments, a targeting moiety comprises a nucleic acid comprising a sequence that at least partially overlaps with a region having genomic coordinates chr4:74595464-74595486, chr4:74595457-74595479, chr4:74595460-74595482, chr4:74595472-74595494, chr4:75000088-75000110, chr4:75000091-75000113, chr4:75000085-75000107, chr4:75000157-75000179, chr4:75000156-75000178, chr4:74595215-74595237, chr4:74595370-74595392, chr4:74595560-74595582, chr4:74595642-74595664, chr4:74595787-74595809, chr4:74528428-74528450, chr4:74528567-74528589, chr4:74528609-74528631, chr4:74789132-74789154, chr4:74789250-74789272, chr4:74789312-74789334, chr4:74964853-74964875, chr4:74964906-74964928, chr4:74965538-74965560, chr4:74965737-74965759, chr4:75000031-75000053, chr4:75000115-75000137, chr4:75000231-75000253, chr4:74975146-74975168, chr4:74975369-74975391, chr4:74976318-74976340, chr4:74570348-74570370, chr4:74570503-74570525, chr4:74570526-74570548, chr5:90785724-90785746, chr5:90788137-90788159, chr5:90908926-90908948, chr5:90661492-90661514, chr5:90661646-90661668, chr5:90661744-90661766, chr5:90785610-90785632, chr5:90909047-90909069, or chr6:113076028-113076047 or a sequence that is within 5, 10, 15, 20, 30, 40, or 50 nucleotides of said region, or comprises a sequence having at least 70, 75, 80, 85, 90, 91, 92, 93, 94, 95, 96, 97, 98, or 99% identity to a sequence at said genomic region, or differing at no more than 1, 2, 3, 4, or 5 positions relative thereto.

In some embodiments, a targeting moiety binds to a sequence at genomic position chr4:74595464-74595486, chr4:74595457-74595479, chr4:74595460-74595482, chr4:74595472-74595494, chr4:75000088-75000110, chr4:75000091-75000113, chr4:75000085-75000107, chr4:75000157-75000179, chr4:75000156-75000178, chr4:74595215-74595237, chr4:74595370-74595392, chr4:74595560-74595582, chr4:74595642-74595664, chr4:74595787-74595809, chr4:74528428-74528450, chr4:74528567-74528589, chr4:74528609-74528631, chr4:74789132-74789154, chr4:74789250-74789272, chr4:74789312-74789334, chr4:74964853-74964875, chr4:74964906-74964928, chr4:74965538-74965560, chr4:74965737-74965759, chr4:75000031-75000053, chr4:75000115-75000137, chr4:75000231-75000253, chr4:74975146-74975168, chr4:74975369-74975391, chr4:74976318-74976340, chr4:74570348-74570370, chr4:74570503-74570525, chr4:74570526-74570548, chr5:90661492-90661514, chr5:90661646-90661668, chr5:90661744-90661766, chr5:90785610-90785632, chr5:90909047-90909069, chr5:90785724-90785746, chr5:90788137-90788159, chr5:90908926-90908948, or chr6:113076028-113076047.

In some embodiments, a targeting moiety binds to an anchor sequence or to a site proximal to an anchor sequence, e.g., an anchor sequence that is part of an ASMC comprising, wholly or in part, a target plurality of genes.

### CRISPR/Cas Targeting Moieties

In some embodiments, a targeting moiety comprises a CRISPR/Cas molecule. In some embodiments, an effector moiety comprises a CRISPR/Cas molecule. A CRISPR/Cas molecule comprises a protein involved in the clustered regulatory interspaced short palindromic repeat (CRISPR) system, e.g., a Cas protein, and optionally a guide RNA, e.g., single guide RNA (sgRNA).

CRISPR systems are adaptive defense systems originally discovered in bacteria and archaea. CRISPR systems use RNA-guided nucleases termed CRISPR-associated or “Cas” endonucleases (e.g., Cas9 or Cpf1) to cleave foreign DNA. For example, in a typical CRISPR/Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. crRNA also contains a region that binds to the tracrRNA to form a partially double-stranded structure which is cleaved by RNase III, resulting in a crRNA/tracrRNA hybrid. A crRNA/tracrRNA hybrid then directs Cas9 endonuclease to recognize and cleave a target DNA sequence. A target DNA sequence must generally be adjacent to a “protospacer adjacent motif” (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e. g., 5′-NGG (e.g., TGG, e.g., CGG, e.g., AGG), and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words, a Cpf1 system requires only Cpf1 nuclease and a crRNA to cleave a target DNA sequence. Cpf1 endonucleases, are associated with T-rich PAM sites, e. g., 5′-TTN. Cpf1 can also recognize a 5′-CTA PAM motif. Cpf1 cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a targeting moiety includes a sequence targeting polypeptide, such as a Cas protein, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus (e.g., a S. pyogenes, or a S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter.

In some embodiments, a Cas protein requires a protospacer adjacent motif (PAM) to be present in or adjacent to a target DNA sequence for the Cas protein to bind and/or function. In some embodiments, the PAM is or comprises, from 5′ to 3′, NGG, YG, NNGRRT, NNNRRT, NGA, TYCV, TATV, NTTN, or NNNGATT, where N stands for any nucleotide, Y stands for C or T, R stands for A or G, and V stands for A or C or G. In some embodiments, a Cas protein is a protein listed in Table 1. In some embodiments, a Cas protein comprises one or more mutations altering its PAM. In some embodiments, a Cas protein comprises E1369R, E1449H, and R1556A mutations or analogous substitutions to the amino acids corresponding to said positions. In some embodiments, a Cas protein comprises E782K, N968K, and R1015H mutations or analogous substitutions to the amino acids corresponding to said positions. In some embodiments, a Cas protein comprises D1135V, R1335Q, and T1337R mutations or analogous substitutions to the amino acids corresponding to said positions. In some embodiments, a Cas protein comprises S542R and K607R mutations or analogous substitutions to the amino acids corresponding to said positions. In some embodiments, a Cas protein comprises S542R, K548V, and N552R mutations or analogous substitutions to the amino acids corresponding to said positions.

TABLE 1

| | | | # of | | Mutations to alter | Mutations to make |
|---|---|---|---|---|---|---|
| Name | Enzyme | Species | AAs | PAM | PAM recognition | catalytically dead |
| FnCas9 | Cas9 | Francisella | 1629 | 5′-NGG-3′ | Wt | D11A/H969A/N995A |
| | | novicida | | | | |
| FnCas9 | Cas9 | Francisella | 1629 | 5′-YG-3′ | E1369R/E1449H/R1556A | D11A/H969A/N995A |
| RHA | | novicida | | | | |
| SaCas9 | Cas9 | Staphylococcus | 1053 | 5′- | Wt | D10A/H557A |
| | | aureus | | NNGRRT-3′ | | |
| SaCas9 | Cas9 | Staphylococcus | 1053 | 5′- | E782K/N968K/R1015H | D10A/H557A |
| KKH | | aureus | | NNNRRT-3′ | | |
| SpCas9 | Cas9 | Streptococcus | 1368 | 5′-NGG-3′ | Wt | D10A/D839A/ |
| | | pyogenes | | | | H840A/N863A |
| SpCas9 | Cas9 | Streptococcus | 1368 | 5′-NGA-3′ | D1135V/R1335Q/T1337R | D10A/D839A/ |
| VQR | | pyogenes | | | | H840A/N863A |
| AsCpf1 | Cpf1 | Acidaminococcus | 1307 | 5′-TYCV-3′ | S542R/K607R | E993A |
| RR | | sp. BV3L6 | | | | |
| AsCpf1 | Cpf1 | Acidaminococcus | 1307 | 5′-TATV-3′ | S542R/K548V/N552R | E993A |
| RVR | | sp. BV3L6 | | | | |
| FnCpf1 | Cpf1 | Francisella | 1300 | 5′-NTTN-3′ | Wt | D917A/E1006A/D1255A |
| | | novicida | | | | |
| NmCas9 | Cas9 | Neisseria | 1082 | 5′- | Wt | D16A/D587A/ |
| | | meningitidis | | NNNGATT-3′ | | H588A/N611A |

In some embodiments, the Cas protein is catalytically active and cuts one or both strands of the target DNA site. In some embodiments, cutting the target DNA site is followed by formation of an alteration, e.g., an insertion or deletion, e.g., by the cellular repair machinery.

In some embodiments, the Cas protein is modified to deactivate the nuclease, e.g., nuclease-deficient Cas9. Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut target DNA. In some embodiments, dCas9 binding to a DNA sequence may interfere with transcription at that site by steric hindrance. In some embodiments, dCas9 binding to an anchor sequence may interfere with (e.g., decrease or prevent) genomic complex (e.g., ASMC) formation and/or maintenance. In some embodiments, a targeting moiety comprises a catalytically inactive Cas9, e.g., dCas9, e.g., Cas9m4. Many catalytically inactive Cas9 proteins are known in the art. In some embodiments, dCas9 comprises mutations in each endonuclease domain of the Cas protein, e.g., D10A and H840A mutations.

In some embodiments, a catalytically inactive Cas9 protein, e.g., dCas9, comprises a D11A mutation or an analogous substitution to the amino acid corresponding to said position. In some embodiments, a catalytically inactive Cas9 protein, e.g., dCas9, comprises a H969A mutation or an analogous substitution to the amino acid corresponding to said position. In some embodiments, a catalytically inactive Cas9 protein, e.g., dCas9, comprises a N995A mutation or an analogous substitution to the amino acid corresponding to said position. In some embodiments, a catalytically inactive Cas9 protein, e.g., dCas9, comprises D11A, H969A, and N995A mutations or analogous substitutions to the amino acids corresponding to said positions.

In some embodiments, a catalytically inactive Cas9 protein, e.g., dCas9, comprises a D10A mutation or an analogous substitution to the amino acid corresponding to said position. In some embodiments, a catalytically inactive Cas9 protein, e.g., dCas9, comprises a H557A mutation or an analogous substitution to the amino acid corresponding to said position. In some embodiments, a catalytically inactive Cas9 protein, e.g., dCas9, comprises D10A and H557A mutations or analogous substitutions to the amino acids corresponding to said positions.

In some embodiments, a catalytically inactive Cas9 protein, e.g., dCas9, comprises a D839A mutation or an analogous substitution to the amino acid corresponding to said position. In some embodiments, a catalytically inactive Cas9 protein, e.g., dCas9, comprises a H840A mutation or an analogous substitution to the amino acid corresponding to said position. In some embodiments, a catalytically inactive Cas9 protein, e.g., dCas9, comprises a N863A mutation or an analogous substitution to the amino acid corresponding to said position. In some embodiments, a catalytically inactive Cas9 protein, e.g., dCas9, comprises D10A, D839A, H840A, and N863A mutations or analogous substitutions to the amino acids corresponding to said positions.

In some embodiments, a catalytically inactive Cas9 protein, e.g., dCas9, comprises a E993A mutation or an analogous substitution to the amino acid corresponding to said position.

In some embodiments, a catalytically inactive Cas9 protein, e.g., dCas9, comprises a D917A mutation or an analogous substitution to the amino acid corresponding to said position. In some embodiments, a catalytically inactive Cas9 protein, e.g., dCas9, comprises a E1006A mutation or an analogous substitution to the amino acid corresponding to said position. In some embodiments, a catalytically inactive Cas9 protein, e.g., dCas9, comprises a D1255A mutation or an analogous substitution to the amino acid corresponding to said position. In some embodiments, a catalytically inactive Cas9 protein, e.g., dCas9, comprises D917A, E1006A, and D1255A mutations or analogous substitutions to the amino acids corresponding to said positions.

In some embodiments, a catalytically inactive Cas9 protein, e.g., dCas9, comprises a D16A mutation or an analogous substitution to the amino acid corresponding to said position. In some embodiments, a catalytically inactive Cas9 protein, e.g., dCas9, comprises a D587A mutation or an analogous substitution to the amino acid corresponding to said position. In some embodiments, a catalytically inactive Cas9 protein, e.g., dCas9, comprises a H588A mutation or an analogous substitution to the amino acid corresponding to said position. In some embodiments, a catalytically inactive Cas9 protein, e.g., dCas9, comprises a N611A mutation or an analogous substitution to the amino acid corresponding to said position. In some embodiments, a catalytically inactive Cas9 protein, e.g., dCas9, comprises D16A, D587A, H588A, and N611A mutations or analogous substitutions to the amino acids corresponding to said positions.

In some aspects, a system described herein comprises, or a method described herein comprises the use of, an expression repressor or a site-specific disrupting agent or a polypeptide comprising one or more (e.g., one) targeting moiety and one or more effector moieties (e.g., one or two effector moieties), wherein the one or more targeting moiety is or comprises a CRISPR/Cas molecule comprising a Cas protein, e.g., catalytically inactive Cas9 protein, e.g., sadCas9, dCas9, e.g., dCas9m4, or a functional variant or fragment thereof. In some embodiments, dCas9 comprises an amino acid sequence of SEQ ID NO: 5, 6, or 7.

### Cas9

(SEQ ID NO: 5)
DKKYSIGLDIGTNSVGWAVITDEYKVPSKKFKVLGNTDRHSIKKN
LIGALLFDSGETAEATRLKRTARRRYTRRKNRICYLQEIFSNEMA
KVDDSFFHRLEESFLVEEDKKHERHPIFGNIVDEVAYHEKYPTIY
HLRKKLVDSTDKADLRLIYLALAHMIKFRGHFLIEGDLNPDNSDV
DKLFIQLVQTYNQLFEENPINASGVDAKAILSARLSKSRRLENLI
AQLPGEKKNGLFGNLIALSLGLTPNFKSNFDLAEDAKLQLSKDTY
DDDLDNLLAQIGDQYADLFLAAKNLSDAILLSDILRVNTEITKAP
LSASMIKRYDEHHQDLTLLKALVRQQLPEKYKEIFFDQSKNGYAG
YIDGGASQEEFYKFIKPILEKMDGTEELLVKLNREDLLRKQRTFD
NGSIPHQIHLGELHAILRRQEDFYPFLKDNREKIEKILTFRIPYY
VGPLARGNSRFAWMTRKSEETITPWNFEEVVDKGASAQSFIERMT
NFDKNLPNEKVLPKHSLLYEYFTVYNELTKVKYVTEGMRKPAFLS
GEQKKAIVDLLFKTNRKVTVKQLKEDYFKKIECFDSVEISGVEDR
FNASLGTYHDLLKIIKDKDFLDNEENEDILEDIVLTLTLFEDREM
IEERLKTYAHLFDDKVMKQLKRRRYTGWGRLSRKLINGIRDKQSG
KTILDFLKSDGFANRNFMQLIHDDSLTFKEDIQKAQVSGQGDSLH
EHIANLAGSPAIKKGILQTVKVVDELVKVMGRHKPENIVIEMARE
NQTTQKGQKNSRERMKRIEEGIKELGSQILKEHPVENTQLQNEKL
YLYYLQNGRDMYVDQELDINRLSDYDVDHIVPQSFLKDDSIDNKV
LTRSDKNRGKSDNVPSEEVVKKMKNYWRQLLNAKLITQRKFDNLT
KAERGGLSELDKAGFIKRQLVETRQITKHVAQILDSRMNTKYDEN
DKLIREVKVITLKSKLVSDFRKDFQFYKVREINNYHHAHDAYLNA
VVGTALIKKYPKLESEFVYGDYKVYDVRKMIAKSEQEIGKATAKY
FFYSNIMNFFKTEITLANGEIRKRPLIETNGETGEIVWDKGRDFA
TVRKVLSMPQVNIVKKTEVQTGGFSKESILPKRNSDKLIARKKDW
DPKKYGGFDSPTVAYSVLVVAKVEKGKSKKLKSVKELLGITIMER
SSFEKNPIDFLEAKGYKEVKKDLIIKLPKYSLFELENGRKRMLAS
AGELQKGNELALPSKYVNFLYLASHYEKLKGSPEDNEQKQLFVEQ
HKHYLDEIIEQISEFSKRVILADANLDKVLSAYNKHRDKPIREQA
ENIIHLFTLTNLGAPAAFKYFDTTIDRKRYTSTKEVLDATLIHQS
ITGLYETRIDLSQLGGD

dCas9 (Cas9m4)

(SEQ ID NO: 6)
DKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMARENQTTQKGQKNSR
ERMKRIEEGIKELGSQILKEHPVENTQLQNEKLYLYYLQNGRDMYVDQELDINRLSDYDVAAIV
PQSFLKDDSIDNKVLTRSDKARGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQEIGKAT
AKYFFYSNIMNFFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKGKSKKLKSVKEL
LGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAY
NKHRDKPIREQAENIIHLFTLTNLGAPAAFKYFDTTIDRKRYTSTKEVLDATLIHQSITGLYETRID
LSQLGGD

### Sa-dCas9

(SEQ ID NO: 7)
AKRNYILGLAIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVHNVNEVE
EDTGNELSTKEQISRNSKALEEKYVAELQLERLKKDGEVRGSINRFKTSDYVKEAKQLLKVQKA
YHQLDQSFIDTYIDLLETRRTYYEGPGEGSPFGWKDIKEWYEMLMGHCTYFPEELRSVKYAYNA
DLYNALNDLNNLVITRDENEKLEYYEKFQIIENVFKQKKKPTLKQIAKEILVNEEDIKGYRVTSTG
KPEFTNLKVYHDIKDITARKEITENAELLDQIAKILTIYQSSEDIQEELTNLNSELTQEEIEQISNLKG
YTGTHNLSLKAINLILDELWHTNDNQIAIFNRLKLVPKKVDLSQQKEIPTTLVDDFILSPVVKRSFI
QSIKVINAIIKKYGLPNDIIIELAREKNSKDAQKMINEMQKRNRQTNERIEEIIRTTGKENAKYLIEK
IKLHDMQEGKCLYSLEAIPLEDLLNNPFNYEVDAIIPRSVSFDNSFNNKVLVKQEENSKKGNRTPF
QYLSSSDSKISYETFKKHILNLAKGKGRISKTKKEYLLEERDINRFSVQKDFINRNLVDTRYATRG
LMNLLRSYFRVNNLDVKVKSINGGFTSFLRRKWKFKKERNKGYKHHAEDALIIANADFIFKEWK
KLDKAKKVMENQMFEEKQAESMPEIETEQEYKEIFITPHQIKHIKDFKDYKYSHRVDKKPNRKLI
NDTLYSTRKDDKGNTLIVNNLNGLYDKDNDKLKKLINKSPEKLLMYHHDPQTYQKLKLIMEQY
GDEKNPLYKYYEETGNYLTKYSKKDNGPVIKKIKYYGNKLNAHLDITDDYPNSRNKVVKLSLKP
YRFDVYLDNGVYKFVTVKNLDVIKKENYYEVNSKCYEEAKKLKKISNQAEFIASFYKNDLIKIN
GELYRVIGVNNDLLNRIEVNMIDITYREYLENMNDKRPPHIIKTIASKTQSIKKYSTDILGNLYEVK
SKKHPQIIKKG

In some embodiments, the dCas9 is encoded by a nucleic acid sequence of SEQ ID NO: 8, or 9:

(SEQ ID NO: 8)
GACAAGAAGTACAGCATCGGCCTGGCCATCGGCACCAACAGCGTG
GGCTGGGCCGTGATCACCGACGAGTACAAGGTGCCCAGCAAGAAG
TTCAAGGTGCTGGGCAACACCGACCGGCACAGCATCAAGAAGAAC
CTGATCGGCGCCCTGCTGTTCGACAGCGGCGAGACCGCCGAGGCC
ACCCGGCTGAAGCGGACCGCCCGGCGGCGGTACACCCGGCGGAAG
AACCGGATCTGCTACCTGCAGGAGATCTTCAGCAACGAGATGGCC
AAGGTGGACGACAGCTTCTTCCACCGGCTGGAGGAGAGCTTCCTG
GTGGAGGAGGACAAGAAGCACGAGCGGCACCCCATCTTCGGCAAC
ATCGTGGACGAGGTGGCCTACCACGAGAAGTACCCCACCATCTAC
CACCTGCGGAAGAAGCTGGTGGACAGCACCGACAAGGCCGACCTG
CGGCTGATCTACCTGGCCCTGGCCCACATGATCAAGTTCCGGGGC
CACTTCCTGATCGAGGGCGACCTGAACCCCGACAACAGCGACGTG
GACAAGCTGTTCATCCAGCTGGTGCAGACCTACAACCAGCTGTTC
GAGGAGAACCCCATCAACGCCAGCGGCGTGGACGCCAAGGCCATC
CTGAGCGCCCGGCTGAGCAAGAGCCGGCGGCTGGAGAACCTGATC
GCCCAGCTGCCCGGCGAGAAGAAGAACGGCCTGTTCGGCAACCTG
ATCGCCCTGAGCCTGGGCCTGACCCCCAACTTCAAGAGCAACTTC
GACCTGGCCGAGGACGCCAAGCTGCAGCTGAGCAAGGACACCTAC
GACGACGACCTGGACAACCTGCTGGCCCAGATCGGCGACCAGTAC
GCCGACCTGTTCCTGGCCGCCAAGAACCTGAGCGACGCCATCCTG
CTGAGCGACATCCTGCGGGTGAACACCGAGATCACCAAGGCCCCC
CTGAGCGCCAGCATGATCAAGCGGTACGACGAGCACCACCAGGAC
CTGACCCTGCTGAAGGCCCTGGTGCGGCAGCAGCTGCCCGAGAAG
TACAAGGAGATCTTCTTCGACCAGAGCAAGAACGGCTACGCCGGC
TACATCGACGGCGGCGCCAGCCAGGAGGAGTTCTACAAGTTCATC
AAGCCCATCCTGGAGAAGATGGACGGCACCGAGGAGCTGCTGGTG
AAGCTGAACCGGGAGGACCTGCTGCGGAAGCAGCGGACCTTCGAC
AACGGCAGCATCCCCCACCAGATCCACCTGGGCGAGCTGCACGCC
ATCCTGCGGCGGCAGGAGGACTTCTACCCCTTCCTGAAGGACAAC
CGGGAGAAGATCGAGAAGATCCTGACCTTCCGGATCCCCTACTAC
GTGGGCCCCCTGGCCCGGGGCAACAGCCGGTTCGCCTGGATGACC
CGGAAATCCGAGGAGACCATCACCCCCTGGAACTTCGAGGAGGTG
GTGGACAAGGGCGCCAGCGCCCAGAGCTTCATCGAGCGGATGACC
AACTTCGACAAGAACCTGCCCAACGAGAAGGTGCTGCCCAAGCAC
AGCCTGCTGTACGAGTACTTCACCGTGTACAACGAGCTGACCAAG
GTGAAGTACGTGACCGAGGGCATGCGGAAGCCCGCCTTCCTGAGC
GGCGAGCAGAAGAAGGCCATCGTGGACCTGCTGTTCAAGACCAAC
CGGAAGGTGACCGTGAAGCAGCTGAAGGAGGACTACTTCAAGAAG
ATCGAGTGCTTCGACAGCGTGGAGATCAGCGGCGTGGAGGACCGG
TTCAACGCCAGCCTGGGCACCTACCACGACCTGCTGAAGATCATC
AAGGACAAGGACTTCCTGGACAACGAGGAGAACGAGGACATCCTG
GAGGACATCGTGCTGACCCTGACCCTGTTCGAGGACCGGGAGATG
ATCGAGGAGCGGCTGAAAACCTACGCCCACCTGTTCGACGACAAG
GTGATGAAGCAGCTGAAGCGGCGGCGGTACACCGGCTGGGGCCGG
CTGAGCCGGAAGCTGATCAACGGCATCCGGGACAAGCAGAGCGGC
AAGACCATCCTGGACTTCCTGAAATCCGACGGCTTCGCCAACCGG
AACTTCATGCAGCTGATCCACGACGACAGCCTGACCTTCAAGGAG
GACATCCAGAAGGCCCAGGTGAGCGGCCAGGGCGACAGCCTGCAC
GAGCACATCGCCAACCTGGCCGGCAGCCCCGCCATCAAGAAGGGC
ATCCTGCAGACCGTGAAGGTGGTGGACGAGCTGGTGAAGGTGATG
GGCCGGCACAAGCCCGAGAACATCGTGATCGAGATGGCCCGGGAG
AACCAGACCACCCAGAAGGGCCAGAAGAACAGCCGGGAGCGGATG
AAGCGGATCGAGGAGGGCATCAAGGAGCTGGGCAGCCAGATCCTG
AAGGAGCACCCCGTGGAGAACACCCAGCTGCAGAACGAGAAGCTG
TACCTGTACTACCTGCAGAACGGCCGGGACATGTACGTGGACCAG
GAGCTGGACATCAACCGGCTGAGCGACTACGACGTGGCCGCCATC
GTGCCCCAGAGCTTCCTGAAGGACGACAGCATCGACAACAAGGTG
CTGACCCGGAGCGACAAGGCCCGGGGCAAGAGCGACAACGTGCCC
AGCGAGGAGGTGGTGAAGAAGATGAAGAACTACTGGCGGCAGCTG
CTGAACGCCAAGCTGATCACCCAGCGGAAGTTCGACAACCTGACC
AAGGCCGAGCGGGGCGGCCTGAGCGAGCTGGACAAGGCCGGCTTC
ATCAAGCGGCAGCTGGTGGAGACCCGGCAGATCACCAAGCACGTG
GCCCAGATCCTGGACAGCCGGATGAACACCAAGTACGACGAGAAC
GACAAGCTGATCCGGGAGGTGAAGGTGATCACCCTGAAATCCAAG
CTGGTGAGCGACTTCCGGAAGGACTTCCAGTTCTACAAGGTGCGG
GAGATCAACAACTACCACCACGCCCACGACGCCTACCTGAACGCC
GTGGTGGGCACCGCCCTGATCAAGAAGTACCCCAAGCTGGAGAGC
GAGTTCGTGTACGGCGACTACAAGGTGTACGACGTGCGGAAGATG
ATCGCCAAGAGCGAGCAGGAGATCGGCAAGGCCACCGCCAAGTAC
TTCTTCTACAGCAACATCATGAACTTCTTCAAGACCGAGATCACC
CTGGCCAACGGCGAGATCCGGAAGCGGCCCCTGATCGAGACCAAC
GGCGAGACCGGCGAGATCGTGTGGGACAAGGGCCGGGACTTCGCC
ACCGTGCGGAAGGTGCTGAGCATGCCCCAGGTGAACATCGTGAAG
AAAACCGAGGTGCAGACCGGCGGCTTCAGCAAGGAGAGCATCCTG
CCCAAGCGGAACAGCGACAAGCTGATCGCCCGGAAGAAGGACTGG
GACCCCAAGAAGTACGGCGGCTTCGACAGCCCCACCGTGGCCTAC
AGCGTGCTGGTGGTGGCCAAGGTGGAGAAGGGCAAGAGCAAGAAG
CTGAAATCCGTGAAGGAGCTGCTGGGCATCACCATCATGGAGCGG
AGCAGCTTCGAGAAGAACCCCATCGACTTCCTGGAGGCCAAGGGC
TACAAGGAGGTGAAGAAGGACCTGATCATCAAGCTGCCCAAGTAC
AGCCTGTTCGAGCTGGAGAACGGCCGGAAGCGGATGCTGGCCAGC
GCCGGCGAGCTGCAGAAGGGCAACGAGCTGGCCCTGCCCAGCAAG
TACGTGAACTTCCTGTACCTGGCCAGCCACTACGAGAAGCTGAAG
GGCAGCCCCGAGGACAACGAGCAGAAGCAGCTGTTCGTGGAGCAG
CACAAGCACTACCTGGACGAGATCATCGAGCAGATCAGCGAGTTC
AGCAAGCGGGTGATCCTGGCCGACGCCAACCTGGACAAGGTGCTG
AGCGCCTACAACAAGCACCGGGACAAGCCCATCCGGGAGCAGGCC
GAGAACATCATCCACCTGTTCACCCTGACCAACCTGGGCGCCCCC
GCCGCCTTCAAGTACTTCGACACCACCATCGACCGGAAGCGGTAC
ACCAGCACCAAGGAGGTGCTGGACGCCACCCTGATCCACCAGAGC
ATCACCGGCCTGTACGAGACCCGGATCGACCTGAGCCAGCTGGGC
GGCGAC
(SEQ ID NO: 9)
GCCAAGCGGAACTACATCCTGGGCCTGGCCATCGGCATCACCAGC
GTGGGCTACGGCATCATCGACTACGAGACCCGGGACGTGATCGAC
GCCGGCGTGCGGCTGTTCAAGGAGGCCAACGTGGAGAACAACGAG
GGCCGGCGGAGCAAGCGGGGCGCCCGGCGGCTGAAGCGGCGGCGG
CGGCACCGGATCCAGCGGGTGAAGAAGCTGCTGTTCGACTACAAC
CTGCTGACCGACCACAGCGAGCTGAGCGGCATCAACCCCTACGAG
GCCCGGGTGAAGGGCCTGAGCCAGAAGCTGAGCGAGGAGGAGTTC
AGCGCCGCCCTGCTGCACCTGGCCAAGCGGCGGGGCGTGCACAAC
GTGAACGAGGTGGAGGAGGACACCGGCAACGAGCTGAGCACCAAG
GAGCAGATCAGCCGGAACAGCAAGGCCCTGGAGGAGAAGTACGTG
GCCGAGCTGCAGCTGGAGCGGCTGAAGAAGGACGGCGAGGTGCGG
GGCAGCATCAACCGGTTCAAGACCAGCGACTACGTGAAGGAGGCC
AAGCAGCTGCTGAAGGTGCAGAAGGCCTACCACCAGCTGGACCAG
AGCTTCATCGACACCTACATCGACCTGCTGGAGACCCGGCGGACC
TACTACGAGGGCCCCGGCGAGGGCAGCCCCTTCGGCTGGAAGGAC
ATCAAGGAGTGGTACGAGATGCTGATGGGCCACTGCACCTACTTC
CCCGAGGAGCTGCGGAGCGTGAAGTACGCCTACAACGCCGACCTG
TACAACGCCCTGAACGACCTGAACAACCTGGTGATCACCCGGGAC
GAGAACGAGAAGCTGGAGTACTACGAGAAGTTCCAGATCATCGAG
AACGTGTTCAAGCAGAAGAAGAAGCCCACCCTGAAGCAGATCGCC
AAGGAGATCCTGGTGAACGAGGAGGACATCAAGGGCTACCGGGTG
ACCAGCACCGGCAAGCCCGAGTTCACCAACCTGAAGGTGTACCAC
GACATCAAGGACATCACCGCCCGGAAGGAGATCATCGAGAACGCC
GAGCTGCTGGACCAGATCGCCAAGATCCTGACCATCTACCAGAGC
AGCGAGGACATCCAGGAGGAGCTGACCAACCTGAACAGCGAGCTG
ACCCAGGAGGAGATCGAGCAGATCAGCAACCTGAAGGGCTACACC
GGCACCCACAACCTGAGCCTGAAGGCCATCAACCTGATCCTGGAC
GAGCTGTGGCACACCAACGACAACCAGATCGCCATCTTCAACCGG
CTGAAGCTGGTGCCCAAGAAGGTGGACCTGAGCCAGCAGAAGGAG
ATCCCCACCACCCTGGTGGACGACTTCATCCTGAGCCCCGTGGTG
AAGCGGAGCTTCATCCAGAGCATCAAGGTGATCAACGCCATCATC
AAGAAGTACGGCCTGCCCAACGACATCATCATCGAGCTGGCCCGG
GAGAAGAACAGCAAGGACGCCCAGAAGATGATCAACGAGATGCAG
AAGCGGAACCGGCAGACCAACGAGCGGATCGAGGAGATCATCCGG
ACCACCGGCAAGGAGAACGCCAAGTACCTGATCGAGAAGATCAAG
CTGCACGACATGCAGGAGGGCAAGTGCCTGTACAGCCTGGAGGCC
ATCCCCCTGGAGGACCTGCTGAACAACCCCTTCAACTACGAGGTG
GACGCCATCATCCCCCGGAGCGTGAGCTTCGACAACAGCTTCAAC
AACAAGGTGCTGGTGAAGCAGGAGGAGAACAGCAAGAAGGGCAAC
CGGACCCCCTTCCAGTACCTGAGCAGCAGCGACAGCAAGATCAGC
TACGAGACCTTCAAGAAGCACATCCTGAACCTGGCCAAGGGCAAG
GGCCGGATCAGCAAGACCAAGAAGGAGTACCTGCTGGAGGAGCGG
GACATCAACCGGTTCAGCGTGCAGAAGGACTTCATCAACCGGAAC
CTGGTGGACACCCGGTACGCCACCCGGGGCCTGATGAACCTGCTG
CGGAGCTACTTCCGGGTGAACAACCTGGACGTGAAGGTGaaatcc
ATCAACGGCGGCTTCACCAGCTTCCTGCGGCGGAAGTGGAAGTTC
AAGAAGGAGCGGAACAAGGGCTACAAGCACCACGCCGAGGACGCC
CTGATCATCGCCAACGCCGACTTCATCTTCAAGGAGTGGAAGAAG
CTGGACAAGGCCAAGAAGGTGATGGAGAACCAGATGTTCGAGGAG
AAGCAGGCCGAGAGCATGCCCGAGATCGAGACCGAGCAGGAGTAC
AAGGAGATCTTCATCACCCCCCACCAGATCAAGCACATCAAGGAC
TTCAAGGACTACAAGTACAGCCACCGGGTGGACAAGAAGCCCAAC
CGGAAGCTGATCAACGACACCCTGTACAGCACCCGGAAGGACGAC
AAGGGCAACACCCTGATCGTGAACAACCTGAACGGCCTGTACGAC
AAGGACAACGACAAGCTGAAGAAGCTGATCAACAAGAGCCCCGAG
AAGCTGCTGATGTACCACCACGACCCCCAGACCTACCAGAAGCTG
AAGCTGATCATGGAGCAGTACGGCGACGAGAAGAACCCCCTGTAC
AAGTACTACGAGGAGACCGGCAACTACCTGACCAAGTACAGCAAG
AAGGACAACGGCCCCGTGATCAAGAAGATCAAGTACTACGGCAAC
AAGCTGAACGCCCACCTGGACATCACCGACGACTACCCCAACAGC
CGGAACAAGGTGGTGAAGCTGAGCCTGAAGCCCTACCGGTTCGAC
GTGTACCTGGACAACGGCGTGTACAAGTTCGTGACCGTGAAGAAC
CTGGACGTGATCAAGAAGGAGAACTACTACGAGGTGAACAGCAAG
TGCTACGAGGAGGCCAAGAAGCTGAAGAAGATCAGCAACCAGGCC
GAGTTCATCGCCAGCTTCTACAAGAACGACCTGATCAAGATCAAC
GGCGAGCTGTACCGGGTGATCGGCGTGAACAACGACCTGCTGAAC
CGGATCGAGGTGAACATGATCGACATCACCTACCGGGAGTACCTG
GAGAACATGAACGACAAGCGGCCCCCCCACATCATCAAGACCATC
GCCAGCAAGACCCAGAGCATCAAGAAGTACAGCACCGACATCCTG
GGCAACCTGTACGAGGTGaaatccAAGAAGCACCCCCAGATCATC
AAGAAGGGC

Guide RNA (gRNA)

In some embodiments, a targeting moiety may comprise a Cas molecule comprising or linked (e.g., covalently) to a gRNA. A gRNA is a short synthetic RNA composed of a “scaffold” sequence necessary for Cas-protein binding and a user-defined ˜20 nucleotide targeting sequence for a genomic target. In practice, guide RNA spacer sequences are generally designed to have a length of between 17-24 nucleotides (e.g., 19, 20, or 21 nucleotides) and be complementary to the targeted nucleic acid sequence. In some embodiments the gRNA comprises 3-6 flanking phosphorothioate (PS) linkages, e.g., 3 flanking PS linkages at each end. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective for use with Cas proteins; see, for example, Hendel et al. (2015) Nature Biotechnol., 985-991.

In some embodiments, a gRNA comprises a nucleic acid sequence that is complementary to a DNA sequence associated with a target gene. In some embodiments, the DNA sequence is, comprises, or overlaps an expression control element that is operably linked to the target gene. In some embodiments, a gRNA comprises a nucleic acid sequence that is at least 90, 95, 99, or 100% complementary to a DNA sequence associated with a target gene. In some embodiments, a gRNA for use with a targeting moiety that comprises a Cas molecule is an sgRNA. In some embodiments, a gRNA comprises a sequence selected from Table 8 or Table 9 or a sequence having at least 70, 75, 80, 85, 90, 91, 92, 93, 94, 95, 96, 97, 98, or 99% identity thereto, or differing at no more than 1, 2, 3, 4, or 5 positions relative thereto.

In some embodiments, a gRNA for use with a CRISPR/Cas molecule of an expression repressor specifically binds a target sequence associated with one or more of CXCL1-8 gene expression (e.g., an E1 cRE). Such a gRNA may comprise a target-binding sequence selected from any one of SEQ ID NOs: 90-100.

In some embodiments, an expression repressor comprises a targeting moiety that binds a target site within genomic coordinates chr4: 74591400-74593000 or chr4:74982639-74983600. In some embodiments, an expression repressor comprises a targeting moiety that binds a target site within genomic coordinates chr4: 74591400-74593000. In some embodiments, an expression repressor comprises a targeting moiety that binds a target site within genomic coordinates chr4:74982639-74983600. In some embodiments, the targeting moiety binds a target site chosen from k) GRCh37: chr4:74591768-74591790; l) GRCh37: chr4:74591844-74591866; m) GRCh37: chr4:74591892-74591914; n) GRCh37: chr4:74592088-74592110; o) GRCh37: chr4:74982748-74982770; p) GRCh37: chr4:74982841-74982863; q) GRCh37: chr4:74982882-74982904; r) GRCh37: chr4:74982960-74982982; s) GRCh37: chr4:74983108-74983130; and t) GRCh37: chr4:74983181-74983203. In some embodiments, the targeting moiety binds a target site with genomic coordinates GRCh37: chr4:74591768-74591790. In some embodiments, the targeting moiety binds a target site with genomic coordinates GRCh37: chr4:74591844-74591866. In some embodiments, the targeting moiety binds a target site with genomic coordinates GRCh37: chr4:74591892-74591914. In some embodiments, the targeting moiety binds a target site with genomic coordinates GRCh37: chr4:74592088-74592110. In some embodiments, the targeting moiety binds a target site with genomic coordinates GRCh37: chr4:74982748-74982770. In some embodiments, the targeting moiety binds a target site with genomic coordinates GRCh37: chr4:74982841-74982863. In some embodiments, the targeting moiety binds a target site with genomic coordinates GRCh37: chr4:74982882-74982904. In some embodiments, the targeting moiety binds a target site with genomic coordinates GRCh37: chr4:74982960-74982982. In some embodiments, the targeting moiety binds a target site with genomic coordinates GRCh37: chr4:74983108-74983130. In some embodiments, the targeting moiety binds a target site with genomic coordinates GRCh37: chr4:74983181-74983203. As used in this disclosure, the genomic coordinates are based on hg19 human genome reference assembly unless specified otherwise.

In some embodiments, an expression repressor comprises a targeting moiety that binds within 500, 300, 200, 100, or 50 nucleotides upstream or downstream of a target site within genomic coordinates chr4: 74591400-74593000 or chr4:74982639-74983600. In some embodiments, an expression repressor comprises a targeting moiety that binds within 500, 300, 200, 100, or 50 nucleotides upstream or downstream of a target site within genomic coordinates chr4: 74591400-74593000. In some embodiments, an expression repressor comprises a targeting moiety that binds within 500, 300, 200, 100, or 50 nucleotides upstream or downstream of a target site within genomic coordinates chr4:74982639-74983600. In some embodiments, the targeting moiety binds within 500, 300, 200, 100, or 50 nucleotides upstream or downstream of a target site chosen from k) GRCh37: chr4:74591768-74591790; l) GRCh37: chr4:74591844-74591866; m) GRCh37: chr4:74591892-74591914; n) GRCh37: chr4:74592088-74592110: o) GRCh37: chr4:74982748-74982770; p) GRCh37: chr4:74982841-74982863; q) GRCh37: chr4:74982882-74982904; r) GRCh37: chr4:74982960-74982982; s) GRCh37: chr4:74983108-74983130; and t) GRCh37: chr4:74983181-74983203. In some embodiments, the targeting moiety binds within 500, 300, 200, 100, or 50 nucleotides upstream or downstream of a target site with genomic coordinates GRCh37: chr4:74591768-74591790. In some embodiments, the targeting moiety binds within 500, 300, 200, 100, or 50 nucleotides upstream or downstream of a target site with genomic coordinates GRCh37: chr4:74591844-74591866. In some embodiments, the targeting moiety binds within 500, 300, 200, 100, or 50 nucleotides upstream or downstream of a target site with genomic coordinates GRCh37: chr4:74591892-74591914. In some embodiments, the targeting moiety binds within 500, 300, 200, 100, or 50 nucleotides upstream or downstream of a target site with genomic coordinates GRCh37: chr4:74592088-74592110. In some embodiments, the targeting moiety binds within 500, 300, 200, 100, or 50 nucleotides upstream or downstream of a target site with genomic coordinates GRCh37: chr4:74982748-74982770. In some embodiments, the targeting moiety binds within 500, 300, 200, 100, or 50 nucleotides upstream or downstream of a target site with genomic coordinates GRCh37: chr4:74982841-74982863. In some embodiments, the targeting moiety binds within 500, 300, 200, 100, or 50 nucleotides upstream or downstream of a target site with genomic coordinates GRCh37: chr4:74982882-74982904. In some embodiments, the targeting moiety binds within 500, 300, 200, 100, or 50 nucleotides upstream or downstream of a target site with genomic coordinates GRCh37: chr4:74982960-74982982. In some embodiments, the targeting moiety binds within 500, 300, 200, 100, or 50 nucleotides upstream or downstream of a target site with genomic coordinates GRCh37: chr4:74983108-74983130. In some embodiments, the targeting moiety binds within 500, 300, 200, 100, or 50 nucleotides upstream or downstream of a target site with genomic coordinates GRCh37: chr4:74983181-74983203. As used in this disclosure, the genomic coordinates are based on hg19 human genome reference assembly unless specified otherwise.

In some embodiments, the expression repressor is used in combination with a site-specific disrupting agent. In some embodiments, the site-specific disrupting agent comprises a CRISPR/Cas molecule. In some embodiments, a gRNA for use with a targeting moiety of a site-specific disrupting agent that comprises a Cas molecule is an sgRNA. In some embodiments, a gRNA binds to a nucleic acid sequence comprising a sequence selected from Table 4, Table 5, Table 6, Table 7 or a sequence having at least 70, 75, 80, 85, 90, 91, 92, 93, 94, 95, 96, 97, 98, or 99% identity thereto, or differing at no more than 1, 2, 3, 4, or 5 positions relative thereto. For example, in some embodiments, the gRNA binds to a strand of a double stranded DNA, wherein one of the strands of the DNA has a sequence set out in any of Tables 4-7. In some embodiments, a gRNA for use with a CRISPR/Cas molecule of the site-specific disrupting agent specifically binds a target sequence associated with one or more of CXCL1-8 gene expression. Such a gRNA may comprise a target-binding sequence selected from SEQ ID NOs: 20-62.

In some embodiments, a targeting moiety is or comprises a Zn finger domain. A Zn finger domain comprises a Zn finger, e.g., a naturally occurring Zn finger or engineered Zn finger, or fragment thereof. Many Zn fingers are known to those of skill in the art and are commercially available, e.g., from Sigma-Aldrich. Generally, a Zn finger domain comprises a plurality of Zn fingers, wherein each Zn finger recognizes three nucleotides. A Zn finger protein can comprise a Zn finger domain and optionally one or more other domains.

In some embodiments, the zinc finger domain comprises 1, 2, 3, 4, 5, 6, 7, 8, 9, or 10 zinc fingers (and optionally no more than 11, 10, 9, 8, 7, 6, or 5 zinc fingers). In some embodiments, the zinc finger domain comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6- 7, 7-10, 7-9, 7-8, 8-10, 8-9, or 9-10 zinc fingers. In some embodiments, the zinc finger domain comprises 3 or 9 zinc fingers. In some embodiments, the zinc finger domain comprises 3 zinc fingers. In some embodiments, the zinc finger domain comprises 9 zinc fingers. In some embodiments, the zinc finger domain comprises 7 zinc fingers. In certain embodiments, the zinc domain targets a site comprising 21 nucleotides.

In some embodiments, a Zn finger domain comprises a non-naturally occurring Zn finger protein that is engineered to bind to a target DNA sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered Zn finger protein may have a novel binding specificity, compared to a naturally-occurring Zn finger protein. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned International Patent Publication No. WO 02/077227.

Zn finger proteins and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, Zn finger proteins and/or multi-fingered Zn finger proteins may be linked together, e.g., as a fusion protein, using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The Zn finger molecules described herein may include any combination of suitable linkers between the individual zinc finger proteins and/or multi-fingered Zn finger proteins of the Zn finger molecule.

In certain embodiments, the targeting moiety comprises a Zn finger domain comprising a plurality of engineered zinc fingers that bind (in a sequence-specific manner) to a target DNA sequence.

In some embodiments, a Zn finger domain comprises one Zn finger or fragment thereof. In other embodiments, the Zn finger domain comprises a plurality of Zn fingers (or fragments thereof), e.g., 2, 3, 4, 5, 6 or more Zn fingers (and optionally no more than 11, 10, 9, 8, 7, 6, 5, 4, 3, or 2 Zn fingers). In some embodiments, the Zn finger domain comprises at least three Zn fingers. In some embodiments, the Zn finger domain comprises four, five or six Zn fingers. In some embodiments, the Zn finger domain comprises 8, 9, 10, or 11 Zn fingers. In some embodiments, a Zn finger domain comprising three Zn finger proteins recognizes a target DNA sequence comprising 9 or 10 nucleotides. In some embodiments, a Zn finger domain comprising four Zn fingers recognizes a target DNA sequence comprising 12 to 14 nucleotides. In some embodiments, a Zn finger domain comprising six Zn fingers recognizes a target DNA sequence comprising 18 to 21 nucleotides.

In some embodiments, a Zn finger protein comprises a two-handed Zn finger protein. Two handed zinc finger proteins are those proteins in which two clusters of zinc finger proteins are separated by intervening amino acids so that the two zinc finger domains bind to two discontinuous target DNA sequences. An example of a two handed type of zinc finger binding protein is SIP1, where a cluster of four zinc finger proteins is located at the amino terminus of the protein and a cluster of three Zn finger proteins is located at the carboxyl terminus (see Remade, et al. (1999) EMBO Journal 18(18):5073-5084). Each cluster of zinc fingers in these proteins is able to bind to a unique target sequence and the spacing between the two target sequences can comprise many nucleotides.

In some embodiments, an expression repressor comprises a targeting moiety comprising a zinc finger domain of Table 10. In some embodiments, an expression repressor comprises a targeting moiety comprising a zinc finger domain encoded by the nucleic acid sequence of Table 11. In some embodiments, an expression repressor comprises a targeting moiety comprising a zinc finger domain of any one of SEQ ID NOs: 112-121 or 170-175. In some embodiments, the zinc finger domain binds to a sequence of Table 12. In some embodiments, an expression repressor comprises a targeting moiety comprising a zinc finger domain that binds a target site within genomic coordinates chr4: 74591400-74593000 or chr4:74982639-74983600. In some embodiments, an expression repressor comprises a targeting moiety comprising a zinc finger domain that binds a target site within genomic coordinates chr4: 74591400-74593000. In some embodiments, an expression repressor comprises a targeting moiety comprising a zinc finger domain that binds a target site within genomic coordinates chr4:74982639-74983600. In some embodiments, the zinc finger domain binds a target site chosen from a) GRCh37: chr4:74591777-74591797; b) GRCh37: chr4:74591834-74591854; c) GRCh37: chr4:74591896-74591916; d) GRCh37: chr4:74592082-74592102; e) GRCh37: chr4:74592107-74592127; f) GRCh37: chr4:74592156-74592176; g) GRCh37: chr4:74592210-74592230; h) GRCh37: chr4:74592057-74592077; i) GRCh37: chr4:74591977-74591997; j) GRCh37: chr4:74591856-74591876. In some embodiments, the zinc finger domain binds a target site with genomic coordinates GRCh37: chr4:74591777-74591797. In some embodiments, the zinc finger domain binds a target site with genomic coordinates GRCh37: chr4: 74591834-74591854. In some embodiments, the zinc finger domain binds a target site with genomic coordinates GRCh37: chr4: 74591896-74591916. In some embodiments, the zinc finger domain binds a target site with genomic coordinates GRCh37: chr4: 74592082-74592102. In some embodiments, the zinc finger domain binds a target site with genomic coordinates GRCh37: chr4: 74592107-74592127. In some embodiments, the zinc finger domain binds a target site with genomic coordinates GRCh37: chr4: 74592156-74592176. In some embodiments, the zinc finger domain binds a target site with genomic coordinates GRCh37: chr4: 74592210-74592230. In some embodiments, the zinc finger domain binds a target site with genomic coordinates GRCh37: chr4: 74592057-74592077. In some embodiments, the zinc finger domain binds a target site with genomic coordinates GRCh37: chr4: 74591977-74591997. In some embodiments, the zinc finger domain binds a target site with genomic coordinates GRCh37: chr4: 74591856-74591876. As used in this disclosure, the genomic coordinates are based on hg19 human genome reference assembly unless specified otherwise.

In some embodiments, the zinc finger domain comprises an amino acid sequence of SEQ ID NO: 112 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and binds a target site with genomic coordinates GRCh37: chr4:74591777-74591797. In some embodiments, the zinc finger domain comprises an amino acid sequence of SEQ ID NO: 113 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and binds a target site with genomic coordinates GRCh37: chr4: 74591834-74591854. In some embodiments, the zinc finger domain comprises an amino acid sequence of SEQ ID NO: 114 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and binds a target site with genomic coordinates GRCh37: chr4: 74591896-74591916. In some embodiments, the zinc finger domain comprises an amino acid sequence of SEQ ID NO: 115 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and binds a target site with genomic coordinates GRCh37: chr4: 74592082-74592102. In some embodiments, the zinc finger domain comprises an amino acid sequence of SEQ ID NO: 116 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and binds a target site with genomic coordinates GRCh37: chr4: 74592107-74592127. In some embodiments, the zinc finger domain comprises an amino acid sequence of SEQ ID NO: 117 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and binds a target site with genomic coordinates GRCh37: chr4: 74592156-74592176. In some embodiments, the zinc finger domain comprises an amino acid sequence of SEQ ID NO: 118 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and binds a target site with genomic coordinates GRCh37: chr4: 74592210-74592230. In some embodiments, the zinc finger domain comprises an amino acid sequence of SEQ ID NO: 119 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and binds a target site with genomic coordinates GRCh37: chr4: 74592057-74592077. In some embodiments, the zinc finger domain comprises an amino acid sequence of SEQ ID NO: 120 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and binds a target site with genomic coordinates GRCh37: chr4: 74591977-74591997. In some embodiments, the zinc finger domain comprises an amino acid sequence of SEQ ID NO: 121 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and binds a target site with genomic coordinates GRCh37: chr4: 74591856-74591876.

In some embodiments, the zinc finger domain comprises an amino acid sequence of SEQ ID NO: 170 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the zinc finger domain comprises an amino acid sequence of SEQ ID NO: 171 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the zinc finger domain comprises an amino acid sequence of SEQ ID NO: 172 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the zinc finger domain comprises an amino acid sequence of SEQ ID NO: 173 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the zinc finger domain comprises an amino acid sequence of SEQ ID NO: 174 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the zinc finger domain comprises an amino acid sequence of SEQ ID NO: 175 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, an expression repressor comprises a targeting moiety comprising a zinc finger domain of Table 10. In some embodiments, an expression repressor comprises a targeting moiety comprising a zinc finger domain encoded by the nucleic acid sequence of Table 11. It is understood that, in some embodiments, the nucleic acid comprises an RNA sequence in which each position indicated as a T in Table 11 is occupied by a U. In some embodiments, a nucleic acid described herein comprises a sequence set out in Table 11, or a sequence having at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises a targeting moiety comprising a zinc finger domain of any one of SEQ ID NOs: 112-121 or 170-175. In some embodiments, the zinc finger domain binds to a sequence of Table 12. In some embodiments, an expression repressor comprises a targeting moiety comprising a zinc finger domain that binds within 500, 300, 200, 100, or 50 nucleotides upstream or downstream of a target site within genomic coordinates chr4: 74591400-74593000 or chr4:74982639-74983600. In some embodiments, an expression repressor comprises a targeting moiety comprising a zinc finder domain that binds within 500, 300, 200, 100, or 50 nucleotides upstream or downstream of a target site within genomic coordinates chr4: 74591400-74593000. In some embodiments, an expression repressor comprises a targeting moiety comprising a zinc finger domain that binds within 500, 300, 200, 100, or 50 nucleotides upstream or downstream of a target site within genomic coordinates chr4:74982639-74983600. In some embodiments, the zinc finger domain binds within 500, 300, 200, 100, or 50 nucleotides upstream or downstream of a target site chosen from a) GRCh37: chr4:74591777-74591797; b) GRCh37: chr4:74591834-74591854; c) GRCh37: chr4:74591896-74591916; d) GRCh37: chr4:74592082-74592102; e) GRCh37: chr4:74592107-74592127; f) GRCh37: chr4:74592156-74592176; g) GRCh37: chr4:74592210-74592230; h) GRCh37: chr4:74592057-74592077; i) GRCh37: chr4:74591977-74591997; j) GRCh37: chr4:74591856-74591876. In some embodiments, the zinc finger domain binds within 500, 300, 200, 100, or 50 nucleotides upstream or downstream of a target site with genomic coordinates GRCh37: chr4:74591777-74591797. In some embodiments, the zinc finger domain binds within 500, 300, 200, 100, or 50 nucleotides upstream or downstream of a target site with genomic coordinates GRCh37: chr4: 74591834-74591854. In some embodiments, the zinc finger domain binds within 500, 300, 200, 100, or 50 nucleotides upstream or downstream of a target site with genomic coordinates GRCh37: chr4: 74591896-74591916. In some embodiments, the zinc finger domain binds within 500, 300, 200, 100, or 50 nucleotides upstream or downstream of a target site with genomic coordinates GRCh37: chr4: 74592082-74592102. In some embodiments, the zinc finger domain binds within 500, 300, 200, 100, or 50 nucleotides upstream or downstream of a target site with genomic coordinates GRCh37: chr4: 74592107-74592127. In some embodiments, the zinc finger domain binds within 500, 300, 200, 100, or 50 nucleotides upstream or downstream of a target site with genomic coordinates GRCh37: chr4: 74592156-74592176. In some embodiments, the zinc finger domain binds within 500, 300, 200, 100, or 50 nucleotides upstream or downstream of a target site with genomic coordinates GRCh37: chr4: 74592210-74592230. In some embodiments, the zinc finger domain binds within 500, 300, 200, 100, or 50 nucleotides upstream or downstream of a target site with genomic coordinates GRCh37: chr4: 74592057-74592077. In some embodiments, the zinc finger domain binds within 500, 300, 200, 100, or 50 nucleotides upstream or downstream of a target site with genomic coordinates GRCh37: chr4: 74591977-74591997. In some embodiments, the zinc finger domain binds within 500, 300, 200, 100, or 50 nucleotides upstream or downstream of a target site with genomic coordinates GRCh37: chr4: 74591856-74591876. As used in this disclosure, the genomic coordinates are based on hg19 human genome reference assembly unless specified otherwise.

TABLE 10
Exemplary Zinc finger domains, e.g., for use in
expression repressors that further comprise an
effector moiety such as a KRAB moiety

| SEQ ID NO | Description | Sequence |
|---|---|---|
| 112 | ZF34-KRAB | LEPGEKPYKCPECGKSFSHKNALQNHQRTHTGEKPYKCPECGKSF |
| | DBD (MR- | SQRANLRAHQRTHTGEKPYKCPECGKSFSRSDKLVRHQRTHTGEK |
| | 32103) | PYKCPECGKSFSQAGHLASHQRTHTGEKPYKCPECGKSFSRNDAL |
| | | TEHQRTHTGEKPYKCPECGKSFSTSGNLTEHQRTHTGEKPYKCPE |
| | | CGKSFSRSDHLTNHQRTHTGKKTS |
| 113 | ZF35-KRAB | LEPGEKPYKCPECGKSFSSKKALTEHQRTHTGEKPYKCPECGKSF |
| | DBD (MR- | STSHSLTEHQRTHTGEKPYKCPECGKSFSHKNALQNHQRTHTGEK |
| | 32104) | PYKCPECGKSFSQSSNLVRHQRTHTGEKPYKCPECGKSFSRSDHL |
| | | TTHQRTHTGEKPYKCPECGKSFSTKNSLTEHQRTHTGEKPYKCPE |
| | | CGKSFSTTGNLTVHQRTHTGKKTS |
| 114 | ZF36-KRAB | LEPGEKPYKCPECGKSFSTSGNLVRHQRTHTGEKPYKCPECGKSF |
| | DBD (MR- | SRADNLTEHQRTHTGEKPYKCPECGKSFSQSGDLRRHQRTHTGEK |
| | 32105) | PYKCPECGKSFSHKNALQNHQRTHTGEKPYKCPECGKSFSDPGNL |
| | | VRHQRTHTGEKPYKCPECGKSFSQRANLRAHQRTHTGEKPYKCPE |
| | | CGKSFSDCRDLARHQRTHTGKKTS |
| 115 | ZF37-KRAB | LEPGEKPYKCPECGKSFSRNDALTEHQRTHTGEKPYKCPECGKSF |
| | DBD (MR- | SDKKDLTRHQRTHTGEKPYKCPECGKSFSRADNLTEHQRTHTGEK |
| | 32106) | PYKCPECGKSFSHKNALQNHQRTHTGEKPYKCPECGKSFSTSHSL |
| | | TEHQRTHTGEKPYKCPECGKSFSTSGNLVRHQRTHTGEKPYKCPE |
| | | CGKSFSREDNLHTHQRTHTGKKTS |
| 116 | ZF38-KRAB | LEPGEKPYKCPECGKSFSRKDNLKNHQRTHTGEKPYKCPECGKSF |
| | DBD (MR- | SRADNLTEHQRTHTGEKPYKCPECGKSFSDPGALVRHQRTHTGEK |
| | 32107) | PYKCPECGKSFSDKKDLTRHQRTHTGEKPYKCPECGKSFSDPGAL |
| | | VRHQRTHTGEKPYKCPECGKSFSRSDNLVRHQRTHTGEKPYKCPE |
| | | CGKSFSSPADLTRHQRTHTGKKTS |
| 117 | ZF39-KRAB | LEPGEKPYKCPECGKSFSRSDELVRHQRTHTGEKPYKCPECGKSF |
| | DBD (MR- | SQSGDLRRHQRTHTGEKPYKCPECGKSFSQRANLRAHQRTHTGEK |
| | 32108) | PYKCPECGKSFSTHLDLIRHQRTHTGEKPYKCPECGKSFSRSDHL |
| | | TTHQRTHTGEKPYKCPECGKSFSTSGNLTEHQRTHTGEKPYKCPE |
| | | CGKSFSQSGDLRRHQRTHTGKKTS |
| 118 | ZF40-KRAB | LEPGEKPYKCPECGKSFSRNDALTEHQRTHTGEKPYKCPECGKSF |
| | DBD (MR- | SRRDELNVHQRTHTGEKPYKCPECGKSFSRSDELVRHQRTHTGEK |
| | 32109) | PYKCPECGKSFSRADNLTEHQRTHTGEKPYKCPECGKSFSTSHSL |
| | | TEHQRTHTGEKPYKCPECGKSFSERSHLREHQRTHTGEKPYKCPE |
| | | CGKSFSTHLDLIRHQRTHTGKKTS |
| 119 | ZF41-KRAB | LEPGEKPYKCPECGKSFSTSGNLTEHQRTHTGEKPYKCPECGKSF |
| | DBD (MR- | SQSSSLVRHQRTHTGEKPYKCPECGKSFSRSDHLTTHQRTHTGEK |
| | 32110) | PYKCPECGKSFSRSDHLTTHQRTHTGEKPYKCPECGKSFSQSSSL |
| | | VRHQRTHTGEKPYKCPECGKSFSSKKALTEHQRTHTGEKPYKCPE |
| | | CGKSFSTKNSLTEHQRTHTGKKTS |
| 120 | ZF42-KRAB | LEPGEKPYKCPECGKSFSTHLDLIRHQRTHTGEKPYKCPECGKSF |
| | DBD (MR- | SDPGNLVRHQRTHTGEKPYKCPECGKSFSRKDNLKNHQRTHTGEK |
| | 32111) | PYKCPECGKSFSDKKDLTRHQRTHTGEKPYKCPECGKSFSRSDEL |
| | | VRHQRTHTGEKPYKCPECGKSFSTHLDLIRHQRTHTGEKPYKCPE |
| | | CGKSFSQSSSLVRHQRTHTGKKTS |
| 121 | ZF43-KRAB | LEPGEKPYKCPECGKSFSTSHSLTEHQRTHTGEKPYKCPECGKSF |
| | DBD (MR- | SQAGHLASHQRTHTGEKPYKCPECGKSFSTKNSLTEHQRTHTGEK |
| | 32112) | PYKCPECGKSFSDCRDLARHQRTHTGEKPYKCPECGKSFSQAGHL |
| | | ASHQRTHTGEKPYKCPECGKSFSQSSSLVRHQRTHTGEKPYKCPE |
| | | CGKSFSRSDHLTTHQRTHTGKKTS |
| 170 | ZF44-KRAB | LEPGEKPYKCPECGKSFSDSGNLRVHQRTHTGEKPYKCPEC |
| | DBD (MR- | GKSFSQLAHLRAHQRTHTGEKPYKCPECGKSFSTKNSLTEH |
| | 32694) | QRTHTGEKPYKCPECGKSFSTSGSLVRHQRTHTGEKPYKCP |
| | | ECGKSFSSPADLTRHQRTHTGEKPYKCPECGKSFSQSSSLV |
| | | RHQRTHTGEKPYKCPECGKSFSQSGDLRRHQRTHTGKKTS |
| 171 | ZF45-KRAB | LEPGEKPYKCPECGKSFSRSDNLVRHQRTHTGEKPYKCPEC |
| | DBD (MR- | GKSFSQNSTLTEHQRTHTGEKPYKCPECGKSFSQSSSLVRH |
| | 32695) | QRTHTGEKPYKCPECGKSFSQKSSLIAHQRTHTGEKPYKCP |
| | | ECGKSFSTSGNLVRHQRTHTGEKPYKCPECGKSFSSPADLT |
| | | RHQRTHTGEKPYKCPECGKSFSRSDELVRHQRTHTGKKTS |
| 172 | ZF46-KRAB | LEPGEKPYKCPECGKSFSTSGSLVRHQRTHTGEKPYKCPEC |
| | DBD (MR- | GKSFSQRANLRAHQRTHTGEKPYKCPECGKSFSRTDTLRDH |
| | 32696) | QRTHTGEKPYKCPECGKSFSRRDELNVHQRTHTGEKPYKCP |
| | | ECGKSFSHRTTLTNHQRTHTGEKPYKCPECGKSFSRSDKLT |
| | | EHQRTHTGEKPYKCPECGKSFSQKSSLIAHQRTHTGKKTS |
| 173 | ZF47-KRAB | LEPGEKPYKCPECGKSFSRADNLTEHQRTHTGEKPYKCPEC |
| | DBD (MR- | GKSFSRSDHLTTHQRTHTGEKPYKCPECGKSFSRSDNLVRH |
| | 32697) | QRTHTGEKPYKCPECGKSFSQNSTLTEHQRTHTGEKPYKCP |
| | | ECGKSFSQSSSLVRHQRTHTGEKPYKCPECGKSFSQKSSLI |
| | | AHQRTHTGEKPYKCPECGKSFSTSGNLVRHQRTHTGKKTS |
| 174 | ZF48-KRAB | LEPGEKPYKCPECGKSFSRSDHLTTHQRTHTGEKPYKCPEC |
| | DBD (MR- | GKSFSDPGHLVRHQRTHTGEKPYKCPECGKSFSHKNALQNH |
| | 32698) | QRTHTGEKPYKCPECGKSFSRRDELNVHQRTHTGEKPYKCP |
| | | ECGKSFSQRANLRAHQRTHTGEKPYKCPECGKSFSTTGNLT |
| | | VHQRTHTGEKPYKCPECGKSFSSKKHLAEHQRTHTGKKTS |
| 175 | ZF49-KRAB | LEPGEKPYKCPECGKSFSSKKALTEHQRTHTGEKPYKCPEC |
| | DBD (MR- | GKSFSTSGNLTEHQRTHTGEKPYKCPECGKSFSHRTTLTNH |
| | 32699) | QRTHTGEKPYKCPECGKSFSRNDALTEHQRTHTGEKPYKCP |
| | | ECGKSFSDSGNLRVHQRTHTGEKPYKCPECGKSFSQSGDLR |
| | | RHQRTHTGEKPYKCPECGKSFSRSDHLTNHQRTHTGKKTS |

TABLE 11
Exemplary nucleic acids encoding zinc finger domains

| SEQ ID NO | Description | Sequence |
|---|---|---|
| 122 | ZF34-KRAB | CTGGAGCCTGGGGAGAAACCCTACAAATGCCCTGAGTGCGGGAAG |
| | DBD RNA | AGTTTCAGCCACAAGAACGCGTTGCAGAACCACCAAAGGACCCAT |
| | (MR-32103) | ACTGGAGAGAAACCGTACAAGTGCCCTGAATGTGGAAAGTCGTTC |
| | | TCCCAACGCGCCAACCTGAGAGCTCACCAACGCACTCATACCGGG |
| | | GAAAAGCCCTATAAGTGCCCGGAATGCGGCAAAAGCTTCTCCCGG |
| | | TCCGACAAGCTTGTGCGGCACCAGCGGACTCACACTGGGGAGAAG |
| | | CCCTACAAGTGCCCAGAATGCGGAAAGAGCTTCAGCCAGGCCGGT |
| | | CATCTGGCCTCGCATCAGCGGACCCACACTGGCGAAAAGCCTTAC |
| | | AAGTGTCCGGAGTGCGGAAAGTCCTTCTCCCGGAACGACGCACTC |
| | | ACCGAACACCAGCGCACCCACACCGGAGAGAAGCCGTATAAGTGT |
| | | CCAGAATGTGGCAAATCCTTCTCCACATCTGGCAATCTGACCGAG |
| | | CACCAGAGAACCCATACCGGAGAAAAGCCGTACAAATGTCCCGAA |
| | | TGCGGGAAGTCATTTTCCCGCTCGGATCACCTGACCAACCACCAG |
| | | AGGACTCACACGGGAAAGAAAACCTCA |
| 123 | ZF35-KRAB | CTTGAGCCCGGCGAAAAGCCCTACAAGTGCCCGGAATGCGGAAAA |
| | DBD RNA | TCGTTCAGCTCAAAGAAGGCCTTGACTGAACACCAACGGACCCAC |
| | (MR-32104) | ACGGGAGAAAAACCGTATAAGTGTCCAGAGTGCGGGAAGTCATTC |
| | | TCCACTTCCCACTCCCTGACCGAGCACCAGCGCACTCACACCGGA |
| | | GAGAAGCCTTACAAGTGTCCTGAGTGCGGAAAGTCTTTCAGCCAC |
| | | AAGAACGCGCTGCAGAACCACCAGAGGACACATACCGGCGAAAAA |
| | | CCCTACAAATGCCCGGAGTGTGGAAAGTCCTTCTCGCAATCCTCC |
| | | AATCTGGTCCGGCACCAGAGAACCCATACTGGGGAAAAACCTTAC |
| | | AAATGTCCGGAGTGCGGCAAAAGCTTTTCCCGCTCCGACCACCTC |
| | | ACCACTCACCAAAGAACCCACACTGGGGAGAAGCCCTATAAGTGC |
| | | CCTGAGTGTGGGAAGTCCTTCAGCACCAAGAACTCGCTCACCGAA |
| | | CACCAGCGGACTCATACCGGAGAAAAGCCGTACAAGTGCCCAGAA |
| | | TGTGGAAAGAGCTTCTCGACCACCGGCAACCTGACTGTGCATCAG |
| | | CGCACCCATACCGGCAAAAAGACCTCA |
| 124 | ZF36-KRAB | CTGGAGCCCGGAGAAAAACCCTATAAGTGTCCGGAGTGCGGAAAG |
| | DBD RNA | TCGTTCTCCACTTCCGGCAACCTTGTGCGGCACCAGCGAACACAT |
| | (MR-32105) | ACCGGAGAAAAGCCCTACAAGTGCCCTGAGTGTGGGAAGTCCTTC |
| | | TCCCGGGCGGACAACTTGACCGAGCACCAGAGAACCCATACTGGG |
| | | GAAAAGCCGTATAAGTGCCCCGAATGTGGAAAGTCCTTTAGCCAA |
| | | AGCGGCGATCTCAGGCGGCATCAGCGCACCCACACTGGCGAAAAG |
| | | CCATACAAGTGCCCGGAGTGTGGAAAATCTTTCTCGCACAAGAAC |
| | | GCACTGCAGAATCACCAGCGGACCCATACCGGGGAGAAGCCGTAC |
| | | AAGTGTCCTGAATGCGGAAAGAGCTTCTCCGACCCTGGCAACCTC |
| | | GTGCGCCACCAACGCACTCACACCGGTGAAAAGCCTTACAAGTGC |
| | | CCAGAGTGCGGGAAGTCATTCAGCCAGCGGGCCAACCTGAGAGCC |
| | | CACCAAAGAACCCACACCGGAGAGAAACCCTACAAATGCCCGGAA |
| | | TGCGGCAAATCGTTCTCCGACTGCCGCGATCTGGCTCGGCACCAG |
| | | AGGACTCACACGGGCAAAAAGACCTCA |
| 125 | ZF37-KRAB | CTGGAACCGGGAGAGAAACCCTATAAGTGTCCGGAGTGCGGAAAG |
| | DBD RNA | TCTTTCTCCCGCAACGACGCGCTTACCGAGCACCAGCGCACCCAC |
| | (MR-32106) | ACTGGAGAAAAACCCTACAAATGCCCTGAATGTGGCAAATCATTC |
| | | TCCGATAAGAAGGACTTGACCAGACACCAGCGGACCCACACCGGA |
| | | GAAAAGCCGTATAAGTGCCCCGAGTGTGGGAAGTCGTTCAGCCGG |
| | | GCCGATAACCTGACGGAACACCAGAGAACTCATACCGGGGAGAAG |
| | | CCGTACAAGTGTCCTGAATGCGGGAAGTCCTTTAGCCACAAGAAC |
| | | GCCCTGCAAAACCACCAACGGACCCATACCGGAGAGAAGCCTTAC |
| | | AAGTGCCCGGAATGCGGAAAGAGCTTCTCGACCTCCCATTCGCTG |
| | | ACTGAGCATCAGAGGACACACACCGGCGAAAAGCCATACAAGTGC |
| | | CCTGAGTGCGGCAAATCCTTCTCCACTTCCGGCAATCTCGTGCGG |
| | | CACCAGAGGACTCACACCGGGGAAAAGCCCTACAAGTGCCCAGAA |
| | | TGTGGAAAGTCCTTCAGCCGCGAGGACAACCTCCACACCCATCAG |
| | | CGCACTCACACTGGCAAAAAGACCTCA |
| 126 | ZF38-KRAB | CTGGAGCCTGGCGAAAAGCCTTACAAGTGTCCCGAATGCGGCAAA |
| | DBD RNA | TCGTTCTCACGGAAGGACAACCTTAAGAACCACCAGAGGACCCAT |
| | (MR-32107) | ACCGGAGAAAAGCCATACAAATGCCCGGAGTGCGGGAAGTCCTTC |
| | | TCCCGCGCCGACAACCTGACTGAGCACCAACGGACCCATACAGGG |
| | | GAGAAGCCCTATAAGTGCCCGGAATGTGGAAAGTCGTTCTCGGAC |
| | | CCCGGTGCACTCGTGCGCCATCAGAGGACGCACACCGGGGAAAAG |
| | | CCCTACAAATGTCCGGAATGCGGAAAGTCCTTTTCCGACAAGAAG |
| | | GATTTGACCAGACACCAGCGCACTCACACCGGAGAGAAGCCGTAC |
| | | AAGTGCCCAGAGTGCGGCAAAAGCTTCTCCGATCCTGGAGCTCTG |
| | | GTCCGGCATCAGCGGACCCACACCGGTGAAAAGCCGTATAAGTGT |
| | | CCGGAGTGTGGAAAGAGCTTCTCCCGGTCCGACAATCTCGTGCGG |
| | | CACCAACGCACCCATACTGGCGAAAAACCCTACAAGTGCCCTGAG |
| | | TGCGGAAAAAGCTTCAGCTCTCCTGCCGACCTGACTAGACACCAG |
| | | AGAACCCACACTGGCAAAAAGACCTCA |
| 127 | ZF39-KRAB | CTGGAACCCGGAGAGAAGCCGTATAAGTGTCCTGAATGCGGAAAG |
| | DBD RNA | TCCTTCTCCCGCTCGGACGAGCTTGTGCGGCACCAGCGCACTCAC |
| | (MR-32108) | ACTGGCGAAAAGCCGTACAAATGCCCCGAATGCGGGAAGTCCTTC |
| | | AGCCAATCGGGAGATCTGCGCAGACATCAGCGGACCCACACCGGA |
| | | GAAAAGCCTTATAAGTGCCCTGAGTGCGGGAAATCGTTCTCCCAA |
| | | CGGGCGAACCTCAGGGCCCACCAACGGACTCACACGGGGGAAAAG |
| | | CCCTACAAGTGTCCGGAGTGTGGAAAGTCATTCTCCACCCACCTG |
| | | GACCTGATCAGGCACCAGCGGACACATACCGGAGAGAAACCGTAC |
| | | AAGTGCCCGGAATGTGGAAAGAGCTTCTCAAGATCCGACCACTTG |
| | | ACCACCCATCAGCGCACCCATACTGGGGAGAAGCCTTACAAATGT |
| | | CCCGAATGTGGCAAATCGTTTTCCACCTCCGGCAACCTGACCGAG |
| | | CATCAGAGGACTCACACCGGCGAAAAACCATACAAGTGCCCAGAG |
| | | TGCGGCAAAAGCTTCAGCCAGTCCGGCGATCTCCGGAGACACCAG |
| | | CGAACCCACACTGGAAAGAAAACCTCT |
| 128 | ZF40-KRAB | CTGGAGCCTGGCGAAAAACCGTACAAGTGCCCCGAATGTGGAAAG |
| | DBD RNA | TCTTTCTCCCGCAACGACGCGTTGACCGAACACCAGCGGACCCAT |
| | (MR-32109) | ACCGGTGAAAAGCCTTACAAGTGCCCTGAGTGCGGCAAAAGCTTC |
| | | TCCCGCCGCGATGAACTGAACGTGCACCAACGCACCCACACTGGA |
| | | GAAAAGCCATACAAATGCCCGGAGTGTGGGAAGTCCTTCTCCCGG |
| | | TCCGATGAGCTTGTGCGGCACCAGAGAACTCACACAGGGGAAAAG |
| | | CCCTATAAGTGCCCAGAGTGCGGGAAGTCATTCAGCCGGGCCGAC |
| | | AACCTGACCGAGCACCAAAGGACTCACACCGGAGAGAAGCCGTAT |
| | | AAGTGTCCTGAGTGTGGAAAGAGCTTCAGCACCTCCCACTCCCTC |
| | | ACCGAACATCAGCGGACTCATACTGGGGAGAAGCCCTACAAGTGT |
| | | CCCGAATGCGGCAAATCCTTTTCGGAACGGTCGCACCTGAGAGAG |
| | | CATCAGAGGACGCACACCGGCGAAAAGCCGTACAAATGTCCGGAG |
| | | TGCGGAAAGTCGTTCTCCACCCACCTCGACCTGATCAGACACCAG |
| | | CGCACCCATACTGGCAAAAAGACCTCA |
| 129 | ZF41-KRAB | CTGGAGCCCGGAGAGAAACCGTACAAGTGTCCAGAGTGCGGCAAA |
| | DBD RNA | TCCTTCTCGACCTCCGGCAACCTGACTGAACACCAGCGCACTCAC |
| | (MR-32110) | ACTGGAGAAAAGCCGTACAAATGTCCTGAATGCGGAAAGAGCTTC |
| | | AGCCAGTCCTCCTCACTTGTGCGCCACCAGAGGACCCACACCGGA |
| | | GAAAAACCGTATAAGTGTCCTGAGTGTGGGAAGTCATTCTCAAGA |
| | | AGCGATCACCTCACCACCCATCAGCGGACCCATACGGGGGAGAAG |
| | | CCCTACAAATGCCCGGAGTGCGGAAAGTCGTTCAGCCGGTCCGAC |
| | | CACCTGACCACCCACCAAAGAACTCACACCGGCGAAAAGCCTTAC |
| | | AAGTGCCCTGAATGTGGAAAGTCCTTTTCCCAATCGTCCTCCTTG |
| | | GTCCGGCATCAGAGGACTCACACAGGAGAGAAGCCATACAAGTGC |
| | | CCCGAATGCGGGAAGTCCTTCAGCTCGAAGAAGGCCCTGACTGAG |
| | | CACCAGCGGACTCATACCGGGGAAAAGCCCTATAAGTGCCCGGAA |
| | | TGCGGCAAAAGCTTCTCGACCAAGAACTCCCTCACCGAGCATCAG |
| | | CGCACCCACACTGGCAAAAAGACCTCT |
| 130 | ZF42-KRAB | CTGGAACCCGGAGAGAAGCCCTATAAGTGTCCGGAGTGCGGCAAA |
| | DBD RNA | TCCTTTTCCACCCATCTCGACCTGATTAGGCACCAGAGAACTCAC |
| | (MR-32111) | ACTGGGGAGAAGCCATACAAGTGCCCCGAATGTGGAAAGTCTTTC |
| | | TCCGATCCGGGCAACCTTGTCCGGCACCAGAGGACCCACACGGGC |
| | | GAAAAACCTTATAAGTGCCCTGAGTGTGGGAAGTCCTTCAGCCGG |
| | | AAGGACAACCTGAAGAACCACCAAAGAACACACACCGGAGAAAAG |
| | | CCCTACAAATGCCCAGAGTGCGGGAAGTCGTTCTCCGACAAGAAG |
| | | GATTTGACCCGGCACCAACGGACCCACACTGGCGAAAAGCCGTAC |
| | | AAATGTCCCGAATGCGGAAAGAGCTTCTCGCGCTCCGACGAGCTC |
| | | GTGCGCCATCAGCGGACTCATACCGGAGAGAAACCGTACAAGTGT |
| | | CCTGAGTGCGGAAAGTCCTTCTCAACCCACCTGGACCTGATCAGA |
| | | CACCAGCGCACTCACACCGGGGAAAAGCCTTACAAGTGCCCGGAA |
| | | TGCGGCAAAAGCTTCTCCCAATCGAGCTCCCTCGTGCGGCATCAG |
| | | CGCACCCATACCGGAAAGAAAACCTCA |
| 131 | ZF43-KRAB | TTGGAACCGGGGGAAAAGCCGTACAAGTGCCCCGAATGTGGCAAA |
| | DBD RNA | TCATTCTCTACCTCCCACTCACTGACCGAGCACCAGAGGACCCAC |
| | (MR-32112) | ACCGGAGAGAAACCCTACAAGTGTCCAGAATGCGGAAAGTCCTTC |
| | | TCGCAAGCCGGTCACCTTGCGTCCCACCAACGCACTCATACTGGA |
| | | GAAAAACCATACAAGTGCCCGGAATGCGGCAAAAGCTTCAGCACC |
| | | AAGAACAGCCTGACTGAGCATCAGAGGACTCACACCGGCGAAAAG |
| | | CCCTATAAGTGTCCTGAGTGCGGCAAATCCTTCTCCGATTGTCGG |
| | | GACCTGGCCAGACACCAGCGCACTCACACAGGAGAAAAGCCTTAT |
| | | AAGTGCCCTGAGTGTGGAAAGTCGTTTAGCCAAGCTGGTCACCTG |
| | | GCATCCCATCAGCGGACTCATACCGGGGAGAAGCCTTACAAATGT |
| | | CCGGAGTGCGGGAAGTCGTTCAGCCAGTCCTCCTCCCTCGTGCGG |
| | | CACCAGAGAACCCACACTGGAGAGAAGCCCTACAAATGCCCTGAA |
| | | TGTGGAAAGAGCTTCTCCCGCTCGGACCACCTCACCACGCACCAG |
| | | CGGACCCATACCGGCAAAAAGACCTCA |
| 194 | ZF44-KRAB | CTGGAGCCCGGGGAGAAGCCATATAAGTGTCCCGAATGCGGAAAG |
| | DBD RNA | TCCTTCAGCGACTCCGGCAACCTTAGGGTCCATCAGCGAACCCAC |
| | (MR-32694) | ACGGGGGAGAAACCGTACAAATGCCCGGAGTGTGGGAAGTCCTTT |
| | | TCCCAACTGGCTCACTTGCGCGCGCACCAGAGAACTCATACTGGA |
| | | GAAAAGCCCTACAAGTGTCCGGAATGTGGAAAGAGCTTCAGCACC |
| | | AAGAACTCCCTCACCGAGCACCAGCGGACCCATACCGGAGAAAAA |
| | | CCTTACAAGTGCCCTGAATGCGGAAAGTCCTTCAGCACGTCCGGT |
| | | TCCCTGGTTCGGCACCAGAGGACCCACACTGGAGAGAAGCCTTAC |
| | | AAATGTCCAGAGTGCGGCAAAAGCTTCTCGAGCCCCGCCGATTTG |
| | | ACCCGCCACCAAAGAACTCACACCGGGGAAAAGCCGTACAAGTGC |
| | | CCGGAATGCGGCAAATCCTTCTCACAATCCTCCTCTCTTGTCCGG |
| | | CACCAGCGCACACATACTGGCGAAAAACCATATAAGTGCCCAGAA |
| | | TGCGGGAAGTCGTTTAGCCAGTCGGGCGATCTGAGGAGACATCAG |
| | | CGCACCCACACCGGAAAGAAAACCAGC |
| 195 | ZF45-KRAB | CTGGAGCCCGGCGAAAAGCCCTACAAGTGTCCGGAGTGCGGAAAG |
| | DBD RNA | AGCTTCTCCCGCTCCGATAACCTTGTGCGCCATCAGCGAACCCAC |
| | (MR-32695) | ACGGGGGAGAAACCGTACAAATGCCCGGAGTGTGGGAAGTCCTTT |
| | | TCCCAAAACAGCACCCTCACCGAGCACCAGCGGACCCATACCGGA |
| | | GAAAAACCTTACAAGTGCCCTGAATGCGGAAAGTCCTTCAGCCAA |
| | | TCCAGCTCCCTGGTTCGGCACCAGAGGACCCACACTGGAGAGAAG |
| | | CCTTACAAATGTCCAGAGTGCGGCAAAAGCTTCTCGCAAAAGTCG |
| | | TCGTTGATTGCCCACCAAAGAACTCACACCGGGGAAAAGCCGTAC |
| | | AAGTGCCCGGAATGCGGCAAATCCTTCTCAACTTCCGGCAACCTT |
| | | GTCCGGCACCAGCGCACACATACTGGCGAAAAACCATATAAGTGC |
| | | CCAGAATGCGGGAAGTCGTTTAGCTCCCCCGCTGATCTGACCAGA |
| | | CATCAGCGGACACACACCGGAGAGAAACCCTATAAGTGTCCCGAA |
| | | TGTGGAAAATCCTTCAGCCGCTCGGACGAACTGGTCCGCCATCAG |
| | | CGGACACACACCGGAAAGAAAACTTCA |
| 196 | ZF46-KRAB | CTGGAACCGGGAGAGAAGCCATACAAATGCCCCGAGTGTGGAAAG |
| | DBD RNA | TCCTTTTCGACTTCCGGTTCGCTTGTGCGCCATCAGCGAACCCAC |
| | (MR-32696) | ACGGGGGAGAAACCGTACAAATGCCCGGAGTGTGGGAAGTCCTTT |
| | | TCCCAACGGGCCAACCTGAGAGCCCACCAAAGGACGCATACCGGC |
| | | GAAAAGCCCTACAAGTGTCCCGAATGTGGAAAGAGCTTCTCTCGC |
| | | ACTGACACCCTCCGGGATCACCAGCGGACCCATACCGGAGAAAAA |
| | | CCTTACAAGTGCCCTGAATGCGGAAAGTCCTTCAGCCGGAGGGAT |
| | | GAACTGAACGTCCACCAGAGGACCCACACTGGAGAGAAGCCTTAC |
| | | AAATGTCCAGAGTGCGGCAAAAGCTTCTCGCATCGCACCACCTTG |
| | | ACCAACCACCAAAGAACTCACACCGGGGAAAAGCCGTACAAGTGC |
| | | CCGGAATGCGGCAAATCCTTCTCACGCTCCGACAAGCTTACTGAA |
| | | CACCAGCGCACACATACTGGCGAAAAACCATATAAGTGCCCAGAA |
| | | TGCGGGAAGTCGTTTAGCCAGAAGTCCAGCCTGATTGCTCACCAG |
| | | CGCACCCACACCGGAAAGAAAACCTCC |
| 197 | ZF47-KRAB | CTGGAACCTGGGGAGAAGCCATATAAGTGCCCTGAGTGTGGAAAG |
| | DBD RNA | TCCTTCTCCCGGGCTGACAATCTGACCGAACACCAGCGGACTCAC |
| | (MR-32697) | ACTGGAGAGAAGCCGTACAAATGCCCCGAGTGCGGAAAGTCGTTC |
| | | TCCCGCTCCGACCATCTCACCACCCATCAGAGAACCCACACCGGG |
| | | GAAAAGCCATACAAGTGTCCTGAATGCGGCAAATCATTCAGCCGC |
| | | TCGGACAACCTCGTGCGGCATCAACGAACCCACACGGGAGAGAAA |
| | | CCGTATAAATGCCCGGAGTGCGGCAAAAGCTTTAGCCAGAACTCC |
| | | ACTCTGACGGAGCACCAACGCACCCATACCGGAGAAAAGCCTTAC |
| | | AAGTGCCCAGAGTGCGGTAAAAGCTTCAGCCAGTCCTCCTCACTC |
| | | GTGCGCCACCAGAGAACTCATACTGGCGAAAAGCCGTATAAGTGC |
| | | CCGGAATGTGGAAAGAGCTTCAGTCAGAAGTCCAGCCTGATTGCG |
| | | CACCAGAGGACTCATACCGGCGAAAAACCCTACAAATGTCCGGAA |
| | | TGCGGAAAATCGTTCTCAACTTCCGGCAACCTTGTGCGGCACCAG |
| | | CGCACACACACCGGCAAAAAGACTTCG |
| 198 | ZF48-KRAB | CTCGAACCCGGAGAAAAGCCCTACAAATGCCCAGAGTGCGGAAAG |
| | DBD RNA | AGCTTTTCCCGCTCCGATCATCTCACCACGCACCAGCGGACCCAT |
| | (MR-32698) | ACCGGAGAAAAACCTTACAAGTGCCCTGAATGCGGAAAGTCCTTC |
| | | AGCGACCCCGGTCACCTGGTTCGGCACCAGAGGACCCACACTGGA |
| | | GAGAAGCCTTACAAATGTCCAGAGTGCGGCAAAAGCTTCTCGCAC |
| | | AAGAACGCCTTGCAAAACCACCAACGCACTCATACCGGCGAAAAG |
| | | CCATACAAGTGTCCGGAGTGTGGGAAGTCCTTCTCGCGACGCGAC |
| | | GAACTGAACGTCCACCAAAGAACTCACACCGGGGAAAAGCCGTAC |
| | | AAGTGCCCGGAATGCGGCAAATCCTTCTCACAACGCGCCAACCTT |
| | | AGGGCTCACCAGCGCACACATACTGGCGAAAAACCATATAAGTGC |
| | | CCAGAATGCGGGAAGTCGTTTAGCACGACTGGCAACCTGACCGTC |
| | | CATCAGCGGACACACACCGGAGAGAAACCCTATAAGTGTCCCGAA |
| | | TGTGGAAAATCCTTCAGCTCTAAGAAGCATCTGGCCGAGCACCAG |
| | | AGAACCCACACTGGGAAGAAAACCAGC |
| 199 | ZF49-KRAB | CTGGAGCCTGGAGAGAAGCCGTATAAGTGCCCAGAATGTGGAAAG |
| | DBD RNA | TCATTCAGCTCCAAGAAGGCTCTGACCGAACACCAACGGACGCAC |
| | (MR-32699) | ACCGGAGAAAAACCCTACAAATGCCCTGAATGCGGGAAGTCCTTT |
| | | TCCACCTCCGGCAACCTGACCGAGCACCAGCGGACTCACACTGGA |
| | | GAGAAACCTTACAAGTGCCCTGAGTGTGGAAAGAGCTTCTCCCAT |
| | | CGCACTACCCTTACCAATCACCAACGCACCCATACCGGCGAAAAA |
| | | CCATACAAATGTCCAGAGTGCGGCAAATCCTTCAGCCGCAACGAT |
| | | GCGCTCACCGAGCATCAGCGCACTCATACTGGCGAAAAGCCCTAT |
| | | AAGTGTCCGGAATGCGGAAAGTCGTTCAGCGACTCCGGAAACCTT |
| | | AGAGTGCACCAGAGGACACACACTGGGGAAAAGCCGTACAAGTGT |
| | | CCCGAATGCGGCAAAAGCTTCAGCCAGTCCGGGGATCTGCGGAGG |
| | | CACCAGCGAACCCATACGGGAGAAAAGCCTTATAAATGCCCGGAG |
| | | TGCGGAAAGAGTTTCTCGCGGAGCGACCACCTGACCAACCACCAG |
| | | AGAACCCACACTGGCAAAAAGACCTCG |

TABLE 12
Exemplary Zinc finger domain target sequences, e.g.,
for an expression repressor comprising an
effector moiety, e.g., KRAB

| SEQ ID NO | Description | Sequence |
|---|---|---|
| 132 | ZF34-KRAB Target Sequence (MR-32103) | AGGCATCTGTGAGGGAAAATT |
| 133 | ZF35-KRAB Target Sequence (MR-32104) | AATCCTTGGGAAATTCCACAC |
| 134 | ZF36-KRAB Target Sequence (MR-32105) | GCCAAAGACATTGCACAGGAT |
| 135 | ZF37-KRAB Target Sequence (MR-32106) | TAGGATCCAATTCAGACCCTG |
| 136 | ZF38-KRAB Target Sequence (MR-32107) | ACAGAGGTCACCGTCCAGAAG |
| 137 | ZF39-KRAB Target Sequence (MR-32108) | GCACATTGGACTAAAGCAGTG |
| 138 | ZF40-KRAB Target Sequence (MR-32109) | ACTAGCCCACAGGTGATGCTG |
| 139 | ZF41-KRAB Target Sequence (MR-32110) | CCTCACGTATGGTGGGTACAT |
| 140 | ZF42-KRAB Target Sequence (MR-32111) | GTAACTGTGACCAAGGACACT |
| 141 | ZF43-KRAB Target Sequence (MR-32112) | TGGGTATGAGCCCCTTGACCA |
| 254 | ZF44-KRAB Target Sequence (MR-32694) | GCAGTAACAGTTCCTAGAAAC |
| 255 | ZF45-KRAB Target Sequence (MR-32695) | GTGACAGATATAGTACTAGAG |
| 256 | ZF46-KRAB Target Sequence (MR-32696) | ATACGGAGTATGACGAAAGTT |
| 257 | ZF47-KRAB Target Sequence (MR-32697) | GATATAGTACTAGAGTGGCAG |
| 258 | ZF48-KRAB Target Sequence (MR-32698) | CCCAATAAAATGATTGGCTGG |
| 259 | ZF49-KRAB Target Sequence (MR-32699) | AGGGCAAACCTGAGTCATCAC |

In some aspects, the disclosure provides an expression repressor comprising a first targeting moiety that binds a target site comprising at least 15, 16, 17, 18, 19, 20, 21, 22, 23, 24, 25, 26, 27, 28, or 29, nucleotides of the sequence of any one of SEQ ID NOs: 162 or 163. In some embodiments, the expression repressor comprises a first effector moiety. In some embodiments, the expression repressor is capable of decreasing expression of a CXCL gene.

In some aspects, the disclosure provides an expression repressor comprising a first targeting moiety that binds to a target site, wherein the target site is within an IL-8 promoter. In some embodiments, the target site is within chr4:74606112-7460646, or within a site beginning 2 kb upstream and/or 2 kb downstream of chr4:74606112-7460646. In some embodiments, the expression repressor comprises a first effector moiety. In some embodiments, the expression repressor is capable of decreasing expression of IL-8.

In some aspects, the disclosure provides an expression repressor comprising a first targeting moiety that binds to a target site within genomic coordinates chr4:74606112-7460646 (based on hg19 human genome reference assembly). In some embodiments, the expression repressor comprises a first effector moiety. In some embodiments, the expression repressor is capable of decreasing expression of IL-8.

In some embodiments, a targeting moiety is or comprises a TAL effector molecule. A TAL effector molecule, e.g., a TAL effector molecule that specifically binds a DNA sequence, comprises a plurality of TAL effector domains or fragments thereof, and optionally one or more additional portions of naturally occurring TAL effectors (e.g., N- and/or C-terminal of the plurality of TAL effector domains). Many TAL effectors are known to those of skill in the art and are commercially available, e.g., from Thermo Fisher Scientific.

TALs are natural effector proteins secreted by numerous species of bacterial pathogens including the plant pathogen Xanthomonas which modulates gene expression in host plants and facilitates bacterial colonization and survival. The specific binding of TAL effectors is based on a central repeat domain of tandemly arranged nearly identical repeats of typically 33 or 34 amino acids (the repeat-variable di-residues, RVD domain).

Members of the TAL effectors family differ mainly in the number and order of their repeats. The number of repeats ranges from 1.5 to 33.5 repeats and the C-terminal repeat is usually shorter in length (e.g., about 20 amino acids) and is generally referred to as a “half-repeat”. Each repeat of the TAL effector feature a one-repeat-to-one-base-pair correlation with different repeat types exhibiting different base-pair specificity (one repeat recognizes one base-pair on the target gene sequence). Generally, the smaller the number of repeats, the weaker the protein-DNA interactions. A number of 6.5 repeats has been shown to be sufficient to activate transcription of a reporter gene (Scholze et al., 2010).

Repeat to repeat variations occur predominantly at amino acid positions 12 and 13, which have therefore been termed “hypervariable” and which are responsible for the specificity of the interaction with the target DNA promoter sequence, as shown in Table 2 listing exemplary repeat variable di-residues (RVD) and their correspondence to nucleic acid base targets.

TABLE 2
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

Accordingly, it is possible to modify the repeats of a TAL effector to target specific DNA sequences. Further studies have shown that the RVD NK can target G. Target sites of TAL effectors also tend to include a T flanking the 5′ base targeted by the first repeat, but the exact mechanism of this recognition is not known. More than 113 TAL effector sequences are known to date. Non-limiting examples of TAL effectors from Xanthomonas include, Hax2, Hax3, Hax4, AvrXa7, AvrXa10 and AvrBs3.

Accordingly, the TAL effector domain of the TAL effector molecule of the present disclosure may be derived from a TAL effector from any bacterial species (e.g., Xanthomonas species such as the African strain of Xanthomonas oryzae pv. Oryzae (Yu et al. 2011), Xanthomonas campestris pv. raphani strain 756C and Xanthomonas oryzae pv. oryzicolastrain BLS256 (Bogdanove et al. 2011). As used herein, the TAL effector domain in accordance with the present disclosure comprises an RVD domain as well as flanking sequence(s) (sequences on the N-terminal and/or C-terminal side of the RVD domain) also from the naturally occurring TAL effector. It may comprise more or fewer repeats than the RVD of the naturally occurring TAL effector. The TAL effector molecule of the present disclosure is designed to target a given DNA sequence based on the above code and others known in the art. The number of TAL effector domains (e.g., repeats (monomers or modules)) and their specific sequence are selected based on the desired DNA target sequence. For example, TAL effector domains, e.g., repeats, may be removed or added in order to suit a specific target sequence. In an embodiment, the TAL effector molecule of the present disclosure comprises between 6.5 and 33.5 TAL effector domains, e.g., repeats. In an embodiment, TAL effector molecule of the present disclosure comprises between 8 and 33.5 TAL effector domains, e.g., repeats, e.g., between 10 and 25 TAL effector domains, e.g., repeats, e.g., between 10 and 14 TAL effector domains, e.g., repeats.

In some embodiments, the TAL effector molecule comprises TAL effector domains that correspond to a perfect match to the DNA target sequence. In some embodiments, a mismatch between a repeat and a target base-pair on the DNA target sequence is permitted as along as it allows for the function of the expression repressor or site-specific disrupting agent comprising the TAL effector molecule. In general, TALE binding is inversely correlated with the number of mismatches. In some embodiments, the TAL effector molecule of a site-specific disrupting agent of the present disclosure comprises no more than 7 mismatches, 6 mismatches, 5 mismatches, 4 mismatches, 3 mismatches, 2 mismatches, or 1 mismatch, and optionally no mismatch, with the target DNA sequence. Without wishing to be bound by theory, in general the smaller the number of TAL effector domains in the TAL effector molecule, the smaller the number of mismatches will be tolerated and still allow for the function of the site-specific disrupting agent comprising the TAL effector molecule. The binding affinity is thought to depend on the sum of matching repeat-DNA combinations. For example, TAL effector molecules having TAL effector domains or more may be able to tolerate up to 7 mismatches.

In addition to the TAL effector domains, the TAL effector molecule of the present disclosure may comprise additional sequences derived from a naturally occurring TAL effector. The length of the C-terminal and/or N-terminal sequence(s) included on each side of the TAL effector domain portion of the TAL effector molecule can vary and be selected by one skilled in the art, for example based on the studies of Zhang et al. (2011). Zhang et al., have characterized a number of C-terminal and N-terminal truncation mutants in Hax3 derived TAL-effector based proteins and have identified key elements, which contribute to optimal binding to the target sequence and thus activation of transcription. Generally, it was found that transcriptional activity is inversely correlated with the length of N-terminus. Regarding the C-terminus, an important element for DNA binding residues within the first 68 amino acids of the Hax 3 sequence was identified. Accordingly, in some embodiments, the first 68 amino acids on the C-terminal side of the TAL effector domains of the naturally occurring TAL effector is included in the TAL effector molecule of a site-specific disrupting agent of the present disclosure. Accordingly, in an embodiment, a TAL effector molecule of the present disclosure comprises 1) one or more TAL effector domains derived from a naturally occurring TAL effector; 2) at least 70, 80, 90, 100, 110, 120, 130, 140, 150, 170, 180, 190, 200, 220, 230, 240, 250, 260, 270, 280 or more amino acids from the naturally occurring TAL effector on the N-terminal side of the TAL effector domains; and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector domains.

In some embodiments, an expression repressor comprises a targeting moiety comprising a TAL domain of Table 13. In some embodiments, an expression repressor comprises a targeting moiety comprising a TAL domain encoded by the nucleic acid sequence of Table 14. In some embodiments, a nucleic acid described herein comprises a sequence set out in Table 14, or a sequence having at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises a targeting moiety comprising a TAL domain of any one of SEQ ID NOs: 268-275, or a sequence having at least 80%, 85%, 90%, 95%, 96%, 97%, 98%, 99% identity thereto, or a sequence with no more than 20, 19, 18, 17, 16, 15, 14, 13, 12, 11, 10, 9, 8, 7, 6, 5, 4, 3, 2, or 1 positions of difference thereto. In some embodiments, the TAL domain of binds to a sequence of Table 15 or 15A, or a sequence having at least 80%, 85%, 90%, 95%, 96%, 97%, 98%, 99% identity thereto, or a sequence with no more than 20, 19, 18, 17, 16, 15, 14, 13, 12, 11, 10, 9, 8, 7, 6, 5, 4, 3, 2, or 1 positions of difference thereto. In some embodiments, an expression repressor comprises a targeting moiety comprising a TAL domain that binds a target site within genomic coordinates GRCh37: chr4:74606162-74606184, GRCh37: chr4: 74605723-74606223, or GRCh37: chr4: 74605223-74606223. In some embodiments, an expression repressor comprises a targeting moiety comprising a TAL domain that binds a target site within genomic coordinates GRCh37: chr4:74606039-74606056. In some embodiments, an expression repressor comprises a targeting moiety comprising a TAL domain that binds a target site within genomic coordinates GRCh37: chr4:74606113-74606130. In some embodiments, an expression repressor comprises a targeting moiety comprising a TAL domain that binds a target site within genomic coordinates GRCh37: chr4:74606137-74606154. In some embodiments, an expression repressor comprises a targeting moiety comprising a TAL domain that binds a target site within genomic coordinates GRCh37: chr4:74606150-74606167. In some embodiments, an expression repressor comprises a targeting moiety comprising a TAL domain that binds a target site within genomic coordinates GRCh37: chr4:74591882-74591899. In some embodiments, an expression repressor comprises a targeting moiety comprising a TAL domain that binds a target site within genomic coordinates GRCh37: chr4:74591923-74591940. In some embodiments, an expression repressor comprises a targeting moiety comprising a TAL domain that binds a target site within genomic coordinates GRCh37: chr4:74591897-74591914. In some embodiments, an expression repressor comprises a targeting moiety comprising a TAL domain that binds a target site within genomic coordinates GRCh37: chr4:74591873-74591890. As used in this disclosure, the genomic coordinates are based on hg19 human genome reference assembly unless specified otherwise.

In some embodiments, the TAL domain comprises an amino acid sequence of SEQ ID NO: 260 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 261 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 262 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 263 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 264 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 265 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 266 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 267 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 268 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 269 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 270 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 271 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 272 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 273 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 274 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the TAL domain comprises an amino acid sequence of SEQ ID NO: 275 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, an expression repressor comprises a targeting moiety comprising a TAL domain of Table 13. In some embodiments, an expression repressor comprises a targeting moiety comprising a TAL domain encoded by the nucleic acid sequence of Table 14. It is understood that, in some embodiments, the nucleic acid comprises an RNA sequence in which each position indicated as a T in Table 14 is occupied by a U. In some embodiments, an expression repressor comprises a targeting moiety comprising a TAL domain of any one of SEQ ID NOs: 260-275. In some embodiments, the TAL domain binds to a sequence of Table 15 or 15A. In some embodiments, an expression repressor comprises a targeting moiety comprising a TAL domain that binds within 500, 300, 200, 100, or 50 nucleotides upstream or downstream of a target site within genomic coordinates GRCh37: chr4:74606162-74606184, GRCh37: chr4: 74605723-74606223, or GRCh37: chr4: 74605223-74606223.

In some embodiments, an expression repressor comprises a targeting moiety comprising a TAL domain that binds within 500, 300, 200, 100, or 50 nucleotides upstream or downstream of a target site within genomic coordinates chr4: GRCh37: chr4:74606162-74606184. In some embodiments, an expression repressor comprises a targeting moiety comprising a TAL domain that binds within 500, 300, 200, 100, or 50 nucleotides upstream or downstream of a target site within genomic coordinates GRCh37: chr4: 74605723-74606223. In some embodiments, an expression repressor comprises a targeting moiety comprising a TAL domain that binds within 500, 300, 200, 100, or 50 nucleotides upstream or downstream of a target site within genomic coordinates GRCh37: chr4: 74605223-74606223.

In some embodiments, the TAL domain binds within 500, 300, 200, 100, or 50 nucleotides upstream or downstream of a target site chosen from: i) GRCh37: chr4:74606039-74606056; ii) GRCh37: chr4:74606113-74606130; iii) GRCh37: chr4:74606137-74606154; iv) GRCh37: chr4:74606150-74606167; v) GRCh37: chr4:74591882-74591899; vi) GRCh37: chr4:74591923-74591940; vii) GRCh37: chr4:74591897-74591914; or viii) GRCh37: chr4:74591873-74591890. In some embodiments, the TAL domain binds within 500, 300, 200, 100, or 50 nucleotides upstream or downstream of a target site with genomic coordinates GRCh37: chr4:74606039-74606056. In some embodiments, the TAL domain binds within 500, 300, 200, 100, or 50 nucleotides upstream or downstream of a target site with genomic coordinates GRCh37: chr4:74606113-74606130. In some embodiments, the TAL domain binds within 500, 300, 200, 100, or 50 nucleotides upstream or downstream of a target site with genomic coordinates GRCh37: chr4:74606137-74606154. In some embodiments, the TAL domain binds within 500, 300, 200, 100, or 50 nucleotides upstream or downstream of a target site with genomic coordinates GRCh37: chr4:74606150-74606167. In some embodiments, the TAL domain binds within 500, 300, 200, 100, or 50 nucleotides upstream or downstream of a target site with genomic coordinates GRCh37: chr4:74591882-74591899. In some embodiments, the TAL domain binds within 500, 300, 200, 100, or 50 nucleotides upstream or downstream of a target site with genomic coordinates GRCh37: chr4:74591923-74591940. In some embodiments, the TAL domain binds within 500, 300, 200, 100, or 50 nucleotides upstream or downstream of a target site with genomic coordinates GRCh37: chr4:74591897-74591914. In some embodiments, the TAL domain binds within 500, 300, 200, 100, or 50 nucleotides upstream or downstream of a target site with genomic coordinates GRCh37: chr4:74591873-74591890. As used in this disclosure, the genomic coordinates are based on hg19 human genome reference assembly unless specified otherwise.

TABLE 13
Exemplary TAL domains, e.g., for use in expression
repressors that further comprise an effector
moiety such as a KRAB moiety

| SEQ ID NO | Description | Sequence |
|---|---|---|
| 268 | TAL06 DBD | HMVDLRTLGYSQQQQEKIKPKVRSTVAQHHEALVGHGFTHAHIVA |
| | (from MR- | LSQHPAALGTVAVKYQDMIAALPEATHEAIVGVGKRGAGARALEA |
| | 32712) | LLTVAGELRGPPLQLDTGQLLKIAKRGGVTAVEAVHAWRNALTGA |
| | | PLNLTPEQVVAIASNGGGKQALETVQRLLPVLCQAHGLTPEQVVA |
| | | IASNIGGKQALETVQRLLPVLCQAHGLTPEQVVAIASHDGGKQAL |
| | | ETVQRLLPVLCQAHGLTPEQVVAIASNGGGKQALETVQRLLPVLC |
| | | QAHGLTPEQVVAIASNNGGKQALETVQRLLPVLCQAHGLTPEQVV |
| | | AIASNIGGKQALETVQRLLPVLCQAHGLTPEQVVAIASNIGGKQA |
| | | LETVQRLLPVLCQAHGLTPEQVVAIASNNGGKQALETVQRLLPVL |
| | | CQAHGLTPEQVVAIASHDGGKQALETVQRLLPVLCQAHGLTPEQV |
| | | VAIASNGGGKQALETVQRLLPVLCQAHGLTPEQVVAIASHDGGKQ |
| | | ALETVQRLLPVLCQAHGLTPEQVVAIASHDGGKQALETVQRLLPV |
| | | LCQAHGLTPEQVVAIASNIGGKQALETVQRLLPVLCQAHGLTPEQ |
| | | VVAIASHDGGKQALETVQRLLPVLCQAHGLTPEQVVAIASNIGGK |
| | | QALETVQRLLPVLCQAHGLTPEQVVAIASNIGGKQALETVQRLLP |
| | | VLCQAHGLTPEQVVAIASNGGGKQALETVQRLLPVLCQAHGLTPE |
| | | QVVAIASNGGGRPALESIVAQLSRPDPALAALTNDHLVALACLGG |
| | | RPALDAVKKGLPHAPALIKRTNRRIPERTSHRV |
| 269 | TAL07 DBD | HMVDLRTLGYSQQQQEKIKPKVRSTVAQHHEALVGHGFTHAHIVA |
| | (from MR- | LSQHPAALGTVAVKYQDMIAALPEATHEAIVGVGKRGAGARALEA |
| | 32713) | LLTVAGELRGPPLQLDTGQLLKIAKRGGVTAVEAVHAWRNALTGA |
| | | PLNLTPEQVVAIASNIGGKQALETVQRLLPVLCQAHGLTPEQVVA |
| | | IASHDGGKQALETVQRLLPVLCQAHGLTPEQVVAIASNGGGKQAL |
| | | ETVQRLLPVLCQAHGLTPEQVVAIASHDGGKQALETVQRLLPVLC |
| | | QAHGLTPEQVVAIASHDGGKQALETVQRLLPVLCQAHGLTPEQVV |
| | | AIASNNGGKQALETVQRLLPVLCQAHGLTPEQVVAIASNGGGKQA |
| | | LETVQRLLPVLCQAHGLTPEQVVAIASNIGGKQALETVQRLLPVL |
| | | CQAHGLTPEQVVAIASNGGGKQALETVQRLLPVLCQAHGLTPEQV |
| | | VAIASNGGGKQALETVQRLLPVLCQAHGLTPEQVVAIASNGGGKQ |
| | | ALETVQRLLPVLCQAHGLTPEQVVAIASNNGGKQALETVQRLLPV |
| | | LCQAHGLTPEQVVAIASNIGGKQALETVQRLLPVLCQAHGLTPEQ |
| | | VVAIASNGGGKQALETVQRLLPVLCQAHGLTPEQVVAIASNIGGK |
| | | QALETVQRLLPVLCQAHGLTPEQVVAIASNIGGKQALETVQRLLP |
| | | VLCQAHGLTPEQVVAIASNNGGKQALETVQRLLPVLCQAHGLTPE |
| | | QVVAIASNNGGRPALESIVAQLSRPDPALAALTNDHLVALACLGG |
| | | RPALDAVKKGLPHAPALIKRTNRRIPERTSHRV |
| 270 | TAL08 DBD | HMVDLRTLGYSQQQQEKIKPKVRSTVAQHHEALVGHGFTHAHIVA |
| | (from MR- | LSQHPAALGTVAVKYQDMIAALPEATHEAIVGVGKRGAGARALEA |
| | 32714) | LLTVAGELRGPPLQLDTGQLLKIAKRGGVTAVEAVHAWRNALTGA |
| | | PLNLTPEQVVAIASNNGGKQALETVQRLLPVLCQAHGLTPEQVVA |
| | | IASNIGGKQALETVQRLLPVLCQAHGLTPEQVVAIASNNGGKQAL |
| | | ETVQRLLPVLCQAHGLTPEQVVAIASNGGGKQALETVQRLLPVLC |
| | | QAHGLTPEQVVAIASHDGGKQALETVQRLLPVLCQAHGLTPEQVV |
| | | AIASNIGGKQALETVQRLLPVLCQAHGLTPEQVVAIASNGGGKQA |
| | | LETVQRLLPVLCQAHGLTPEQVVAIASHDGGKQALETVQRLLPVL |
| | | CQAHGLTPEQVVAIASNIGGKQALETVQRLLPVLCQAHGLTPEQV |
| | | VAIASHDGGKQALETVQRLLPVLCQAHGLTPEQVVAIASNIGGKQ |
| | | ALETVQRLLPVLCQAHGLTPEQVVAIASHDGGKQALETVQRLLPV |
| | | LCQAHGLTPEQVVAIASNGGGKQALETVQRLLPVLCQAHGLTPEQ |
| | | VVAIASNGGGKQALETVQRLLPVLCQAHGLTPEQVVAIASHDGGK |
| | | QALETVQRLLPVLCQAHGLTPEQVVAIASHDGGKQALETVQRLLP |
| | | VLCQAHGLTPEQVVAIASNGGGKQALETVQRLLPVLCQAHGLTPE |
| | | QVVAIASNIGGRPALESIVAQLSRPDPALAALTNDHLVALACLGG |
| | | RPALDAVKKGLPHAPALIKRTNRRIPERTSHRV |
| 271 | TAL09 DBD | HMVDLRTLGYSQQQQEKIKPKVRSTVAQHHEALVGHGFTHAHIVA |
| | (from MR- | LSQHPAALGTVAVKYQDMIAALPEATHEAIVGVGKRGAGARALEA |
| | 32715) | LLTVAGELRGPPLQLDTGQLLKIAKRGGVTAVEAVHAWRNALTGA |
| | | PLNLTPEQVVAIASNNGGKQALETVQRLLPVLCQAHGLTPEQVVA |
| | | IASNIGGKQALETVQRLLPVLCQAHGLTPEQVVAIASHDGGKQAL |
| | | ETVQRLLPVLCQAHGLTPEQVVAIASNGGGKQALETVQRLLPVLC |
| | | QAHGLTPEQVVAIASHDGGKQALETVQRLLPVLCQAHGLTPEQVV |
| | | AIASNIGGKQALETVQRLLPVLCQAHGLTPEQVVAIASNNGGKQA |
| | | LETVQRLLPVLCQAHGLTPEQVVAIASNNGGKQALETVQRLLPVL |
| | | CQAHGLTPEQVVAIASNGGGKQALETVQRLLPVLCQAHGLTPEQV |
| | | VAIASNGGGKQALETVQRLLPVLCQAHGLTPEQVVAIASNGGGKQ |
| | | ALETVQRLLPVLCQAHGLTPEQVVAIASNNGGKQALETVQRLLPV |
| | | LCQAHGLTPEQVVAIASHDGGKQALETVQRLLPVLCQAHGLTPEQ |
| | | VVAIASHDGGKQALETVQRLLPVLCQAHGLTPEQVVAIASHDGGK |
| | | QALETVQRLLPVLCQAHGLTPEQVVAIASNGGGKQALETVQRLLP |
| | | VLCQAHGLTPEQVVAIASNNGGKQALETVQRLLPVLCQAHGLTPE |
| | | QVVAIASNIGGRPALESIVAQLSRPDPALAALTNDHLVALACLGG |
| | | RPALDAVKKGLPHAPALIKRTNRRIPERTSHRV |
| 272 | TAL10 DBD | HMVDLRTLGYSQQQQEKIKPKVRSTVAQHHEALVGHGFTHAHIVA |
| | (from MR- | LSQHPAALGTVAVKYQDMIAALPEATHEAIVGVGKRGAGARALEA |
| | 32913) | LLTVAGELRGPPLQLDTGQLLKIAKRGGVTAVEAVHAWRNALTGA |
| | | PLNLTPEQVVAIASHDGGKQALETVQRLLPVLCQAHGLTPEQVVA |
| | | IASHDGGKQALETVQRLLPVLCQAHGLTPEQVVAIASNGGGKQAL |
| | | ETVQRLLPVLCQAHGLTPEQVVAIASNNGGKQALETVQRLLPVLC |
| | | QAHGLTPEQVVAIASNIGGKQALETVQRLLPVLCQAHGLTPEQVV |
| | | AIASNNGGKQALETVQRLLPVLCQAHGLTPEQVVAIASNNGGKQA |
| | | LETVQRLLPVLCQAHGLTPEQVVAIASHDGGKQALETVQRLLPVL |
| | | CQAHGLTPEQVVAIASNIGGKQALETVQRLLPVLCQAHGLTPEQV |
| | | VAIASNGGGKQALETVQRLLPVLCQAHGLTPEQVVAIASNNGGKQ |
| | | ALETVQRLLPVLCQAHGLTPEQVVAIASNGGGKQALETVQRLLPV |
| | | LCQAHGLTPEQVVAIASHDGGKQALETVQRLLPVLCQAHGLTPEQ |
| | | VVAIASNIGGKQALETVQRLLPVLCQAHGLTPEQVVAIASNNGGK |
| | | QALETVQRLLPVLCQAHGLTPEQVVAIASHDGGKQALETVQRLLP |
| | | VLCQAHGLTPEQVVAIASHDGGKQALETVQRLLPVLCQAHGLTPE |
| | | QVVAIASNIGGRPALESIVAQLSRPDPALAALTNDHLVALACLGG |
| | | RPALDAVKKGLPHAPALIKRTNRRIPERTSHRV |
| 273 | TAL11 DBD | HMVDLRTLGYSQQQQEKIKPKVRSTVAQHHEALVGHGFTHAHIVA |
| | (from MR- | LSQHPAALGTVAVKYQDMIAALPEATHEAIVGVGKRGAGARALEA |
| | 32914) | LLTVAGELRGPPLQLDTGQLLKIAKRGGVTAVEAVHAWRNALTGA |
| | | PLNLTPEQVVAIASHDGGKQALETVQRLLPVLCQAHGLTPEQVVA |
| | | IASNGGGKQALETVQRLLPVLCQAHGLTPEQVVAIASNNGGKQAL |
| | | ETVQRLLPVLCQAHGLTPEQVVAIASNIGGKQALETVQRLLPVLC |
| | | QAHGLTPEQVVAIASNNGGKQALETVQRLLPVLCQAHGLTPEQVV |
| | | AIASNGGGKQALETVQRLLPVLCQAHGLTPEQVVAIASHDGGKQA |
| | | LETVQRLLPVLCQAHGLTPEQVVAIASNIGGKQALETVQRLLPVL |
| | | CQAHGLTPEQVVAIASHDGGKQALETVQRLLPVLCQAHGLTPEQV |
| | | VAIASHDGGKQALETVQRLLPVLCQAHGLTPEQVVAIASNGGGKQ |
| | | ALETVQRLLPVLCQAHGLTPEQVVAIASHDGGKQALETVQRLLPV |
| | | LCQAHGLTPEQVVAIASNGGGKQALETVQRLLPVLCQAHGLTPEQ |
| | | VVAIASHDGGKQALETVQRLLPVLCQAHGLTPEQVVAIASNGGGK |
| | | QALETVQRLLPVLCQAHGLTPEQVVAIASNNGGKQALETVQRLLP |
| | | VLCQAHGLTPEQVVAIASNNGGKQALETVQRLLPVLCQAHGLTPE |
| | | QVVAIASNIGGRPALESIVAQLSRPDPALAALTNDHLVALACLGG |
| | | RPALDAVKKGLPHAPALIKRTNRRIPERTSHRV |
| 274 | TAL12 DBD | HMVDLRTLGYSQQQQEKIKPKVRSTVAQHHEALVGHGFTHAHIVA |
| | (from MR- | LSQHPAALGTVAVKYQDMIAALPEATHEAIVGVGKRGAGARALEA |
| | 32915) | LLTVAGELRGPPLQLDTGQLLKIAKRGGVTAVEAVHAWRNALTGA |
| | | PLNLTPEQVVAIASHDGGKQALETVQRLLPVLCQAHGLTPEQVVA |
| | | IASHDGGKQALETVQRLLPVLCQAHGLTPEQVVAIASNGGGKQAL |
| | | ETVQRLLPVLCQAHGLTPEQVVAIASNNGGKQALETVQRLLPVLC |
| | | QAHGLTPEQVVAIASNGGGKQALETVQRLLPVLCQAHGLTPEQVV |
| | | AIASNNGGKQALETVQRLLPVLCQAHGLTPEQVVAIASHDGGKQA |
| | | LETVQRLLPVLCQAHGLTPEQVVAIASNIGGKQALETVQRLLPVL |
| | | CQAHGLTPEQVVAIASNIGGKQALETVQRLLPVLCQAHGLTPEQV |
| | | VAIASNGGGKQALETVQRLLPVLCQAHGLTPEQVVAIASNNGGKQ |
| | | ALETVQRLLPVLCQAHGLTPEQVVAIASNGGGKQALETVQRLLPV |
| | | LCQAHGLTPEQVVAIASHDGGKQALETVQRLLPVLCQAHGLTPEQ |
| | | VVAIASNGGGKQALETVQRLLPVLCQAHGLTPEQVVAIASNGGGK |
| | | QALETVQRLLPVLCQAHGLTPEQVVAIASNGGGKQALETVQRLLP |
| | | VLCQAHGLTPEQVVAIASNNGGKQALETVQRLLPVLCQAHGLTPE |
| | | QVVAIASNNGGRPALESIVAQLSRPDPALAALTNDHLVALACLGG |
| | | RPALDAVKKGLPHAPALIKRTNRRIPERTSHRV |
| 275 | TAL13 DBD | HMVDLRTLGYSQQQQEKIKPKVRSTVAQHHEALVGHGFTHAHIVA |
| | (from MR- | LSQHPAALGTVAVKYQDMIAALPEATHEAIVGVGKRGAGARALEA |
| | 32916) | LLTVAGELRGPPLQLDTGQLLKIAKRGGVTAVEAVHAWRNALTGA |
| | | PLNLTPEQVVAIASNGGGKQALETVQRLLPVLCQAHGLTPEQVVA |
| | | IASNNGGKQALETVQRLLPVLCQAHGLTPEQVVAIASHDGGKQAL |
| | | ETVQRLLPVLCQAHGLTPEQVVAIASHDGGKQALETVQRLLPVLC |
| | | QAHGLTPEQVVAIASNGGGKQALETVQRLLPVLCQAHGLTPEQVV |
| | | AIASHDGGKQALETVQRLLPVLCQAHGLTPEQVVAIASNIGGKQA |
| | | LETVQRLLPVLCQAHGLTPEQVVAIASNNGGKQALETVQRLLPVL |
| | | CQAHGLTPEQVVAIASNNGGKQALETVQRLLPVLCQAHGLTPEQV |
| | | VAIASNIGGKQALETVQRLLPVLCQAHGLTPEQVVAIASNNGGKQ |
| | | ALETVQRLLPVLCQAHGLTPEQVVAIASNGGGKQALETVQRLLPV |
| | | LCQAHGLTPEQVVAIASHDGGKQALETVQRLLPVLCQAHGLTPEQ |
| | | VVAIASNIGGKQALETVQRLLPVLCQAHGLTPEQVVAIASNGGGK |
| | | QALETVQRLLPVLCQAHGLTPEQVVAIASNNGGKQALETVQRLLP |
| | | VLCQAHGLTPEQVVAIASNNGGKQALETVQRLLPVLCQAHGLTPE |
| | | QVVAIASNNGGRPALESIVAQLSRPDPALAALTNDHLVALACLGG |
| | | RPALDAVKKGLPHAPALIKRTNRRIPERTSHRV |

TABLE 14
Exemplary nucleic acids encoding TAL domains

| SEQ ID NO | Description | Sequence |
|---|---|---|
| 276 | TAL06 DBD | CATATGGTGGACCTGAGAACCCTTGGATACTCCCAGCAGCA |
| | RNA (from | GCAGGAGAAGATTAAGCCCAAGGTCCGGTCGACCGTCGCTC |
| | MR-32712) | AACACCACGAGGCCCTCGTGGGACATGGCTTCACCCACGCA |
| | | CACATTGTCGCGCTGTCCCAACACCCTGCCGCCCTTGGAAC |
| | | TGTGGCCGTCAAGTACCAGGATATGATCGCCGCTCTGCCTG |
| | | AAGCGACGCATGAAGCGATTGTGGGCGTGGGCAAGAGAGGA |
| | | GCAGGAGCCCGAGCACTGGAAGCCCTTCTGACCGTCGCCGG |
| | | CGAACTGAGGGGTCCACCTCTGCAACTGGATACCGGGCAGC |
| | | TGCTGAAGATCGCTAAGAGAGGCGGAGTGACCGCCGTCGAA |
| | | GCCGTCCATGCATGGCGCAACGCGCTGACTGGAGCACCGCT |
| | | GAACCTGACCCCCGAACAAGTCGTTGCCATTGCTAGTAACG |
| | | GTGGCGGCAAGCAGGCTCTGGAAACCGTCCAGCGCCTCCTT |
| | | CCTGTCCTGTGTCAAGCGCACGGGCTCACACCTGAACAAGT |
| | | AGTGGCCATCGCTTCCAACATTGGTGGCAAGCAGGCCCTGG |
| | | AAACTGTACAGCGGTTGTTGCCTGTCCTCTGCCAAGCGCAT |
| | | GGGCTTACCCCGGAGCAAGTGGTCGCTATCGCTTCTCATGA |
| | | TGGTGGGAAGCAGGCGCTGGAAACAGTGCAGAGACTGCTTC |
| | | CGGTGCTGTGCCAGGCACATGGCCTGACGCCGGAACAAGTG |
| | | GTGGCGATTGCATCTAACGGAGGCGGGAAGCAGGCGCTGGA |
| | | GACAGTGCAACGGCTGCTCCCTGTTCTATGCCAAGCTCATG |
| | | GACTCACCCCCGAGCAAGTCGTCGCCATTGCGTCCAACAAT |
| | | GGAGGCAAACAGGCACTGGAAACGGTGCAAAGACTGTTGCC |
| | | TGTACTGTGCCAGGCTCATGGCCTTACTCCCGAACAGGTGG |
| | | TGGCAATTGCGAGTAACATCGGCGGAAAGCAGGCTTTGGAA |
| | | ACTGTGCAGCGCTTGCTGCCTGTGCTCTGTCAGGCTCACGG |
| | | ACTCACCCCCGAGCAAGTCGTAGCCATCGCCTCAAATATCG |
| | | GAGGCAAGCAAGCGCTCGAGACTGTGCAGAGGCTCCTGCCT |
| | | GTGTTATGTCAGGCACACGGGCTGACCCCTGAACAAGTCGT |
| | | GGCCATTGCCTCCAACAACGGCGGAAAACAGGCGCTCGAAA |
| | | CAGTCCAGAGGCTTTTGCCCGTGCTGTGTCAAGCTCACGGC |
| | | CTGACTCCGGAGCAAGTGGTCGCCATTGCATCCCATGATGG |
| | | CGGTAAACAAGCATTGGAAACGGTCCAACGCCTGCTGCCGG |
| | | TCCTGTGCCAGGCCCATGGTCTGACTCCCGAACAGGTCGTG |
| | | GCTATCGCCTCCAACGGAGGTGGAAAACAAGCCCTGGAGAC |
| | | TGTACAGAGGCTGCTGCCCGTGCTATGTCAAGCACATGGAC |
| | | TTACGCCTGAACAAGTGGTGGCAATTGCATCACACGACGGA |
| | | GGGAAGCAAGCCCTTGAAACCGTGCAAAGGCTCCTCCCGGT |
| | | GCTGTGCCAAGCCCATGGATTGACTCCAGAACAGGTGGTCG |
| | | CTATCGCATCACATGACGGTGGAAAGCAAGCCCTCGAAACT |
| | | GTGCAGCGGCTCCTCCCTGTGTTGTGCCAGGCGCACGGTCT |
| | | GACTCCCGAGCAAGTCGTGGCAATCGCCTCGAACATCGGGG |
| | | GCAAACAAGCTCTCGAAACTGTTCAAAGACTGCTCCCCGTG |
| | | CTCTGTCAAGCTCACGGCCTCACTCCTGAACAGGTAGTCGC |
| | | GATTGCCTCCCATGACGGGGGAAAGCAGGCCTTGGAGACAG |
| | | TCCAGCGGCTCTTGCCGGTCTTGTGCCAAGCACACGGATTG |
| | | ACTCCAGAACAGGTGGTCGCAATCGCCAGCAATATCGGGGG |
| | | GAAGCAAGCCTTGGAAACCGTTCAGCGGCTACTCCCTGTGC |
| | | TGTGTCAAGCCCACGGTCTGACCCCAGAACAAGTGGTGGCT |
| | | ATCGCGTCGAACATTGGAGGCAAACAGGCCCTCGAGACTGT |
| | | CCAGAGACTCCTGCCAGTCCTCTGCCAGGCTCATGGGCTGA |
| | | CTCCTGAGCAAGTTGTGGCCATAGCATCCAACGGCGGAGGA |
| | | AAGCAAGCTCTGGAAACCGTACAGAGACTTCTCCCGGTGTT |
| | | GTGTCAGGCCCACGGACTTACTCCGGAACAGGTTGTCGCCA |
| | | TCGCGTCCAACGGTGGTGGCAGGCCTGCTCTGGAATCCATC |
| | | GTGGCACAGTTGTCAAGACCAGATCCTGCCCTGGCTGCGCT |
| | | CACCAACGACCACTTGGTGGCTCTGGCCTGCCTTGGCGGTC |
| | | GCCCTGCCTTGGATGCCGTGAAGAAGGGACTTCCTCACGCC |
| | | CCGGCACTGATTAAGCGCACCAATCGCAGGATCCCTGAACG |
| | | GACCTCGCACAGGGTC |
| 277 | TAL07 DBD | CATATGGTGGACCTGAGAACCCTTGGATACTCCCAGCAGCA |
| | RNA (from | GCAGGAGAAGATTAAGCCCAAGGTCCGGTCGACCGTCGCTC |
| | MR-32713) | AACACCACGAGGCCCTCGTGGGACATGGCTTCACCCACGCA |
| | | CACATTGTCGCGCTGTCCCAACACCCTGCCGCCCTTGGAAC |
| | | TGTGGCCGTCAAGTACCAGGATATGATCGCCGCTCTGCCTG |
| | | AAGCGACGCATGAAGCGATTGTGGGCGTGGGCAAGAGAGGA |
| | | GCAGGAGCCCGAGCACTGGAAGCCCTTCTGACCGTCGCCGG |
| | | CGAACTGAGGGGTCCACCTCTGCAACTGGATACCGGGCAGC |
| | | TGCTGAAGATCGCTAAGAGAGGCGGAGTGACCGCCGTCGAA |
| | | GCCGTCCATGCATGGCGCAACGCGCTGACTGGAGCACCGCT |
| | | GAACCTGACCCCCGAACAAGTCGTTGCCATTGCTAGTAACA |
| | | TTGGCGGCAAGCAGGCTCTGGAAACCGTCCAGCGCCTCCTT |
| | | CCTGTCCTGTGTCAAGCGCACGGGCTCACACCTGAACAAGT |
| | | AGTGGCCATCGCTTCCCATGATGGTGGCAAGCAGGCCCTGG |
| | | AAACTGTACAGCGGTTGTTGCCTGTCCTCTGCCAAGCGCAT |
| | | GGGCTTACCCCGGAGCAAGTGGTCGCTATCGCTTCTAACGG |
| | | CGGTGGGAAGCAGGCGCTGGAAACAGTGCAGAGACTGCTTC |
| | | CGGTGCTGTGCCAGGCACATGGCCTGACGCCGGAACAAGTG |
| | | GTGGCGATTGCATCTCACGATGGCGGGAAGCAGGCGCTGGA |
| | | GACAGTGCAACGGCTGCTCCCTGTTCTATGCCAAGCTCATG |
| | | GACTCACCCCCGAGCAAGTCGTCGCCATTGCGTCCCACGAT |
| | | GGAGGCAAACAGGCACTGGAAACGGTGCAAAGACTGTTGCC |
| | | TGTACTGTGCCAGGCTCATGGCCTTACTCCCGAACAGGTGG |
| | | TGGCAATTGCGAGTAACAACGGCGGAAAGCAGGCTTTGGAA |
| | | ACTGTGCAGCGCTTGCTGCCTGTGCTCTGTCAGGCTCACGG |
| | | ACTCACCCCCGAGCAAGTCGTAGCCATCGCCTCAAATGGAG |
| | | GAGGCAAGCAAGCGCTCGAGACTGTGCAGAGGCTCCTGCCT |
| | | GTGTTATGTCAGGCACACGGGCTGACCCCTGAACAAGTCGT |
| | | GGCCATTGCCTCCAACATCGGCGGAAAACAGGCGCTCGAAA |
| | | CAGTCCAGAGGCTTTTGCCCGTGCTGTGTCAAGCTCACGGC |
| | | CTGACTCCGGAGCAAGTGGTCGCCATTGCATCCAACGGAGG |
| | | CGGTAAACAAGCATTGGAAACGGTCCAACGCCTGCTGCCGG |
| | | TCCTGTGCCAGGCCCATGGTCTGACTCCCGAACAGGTCGTG |
| | | GCTATCGCCTCCAACGGAGGTGGAAAACAAGCCCTGGAGAC |
| | | TGTACAGAGGCTGCTGCCCGTGCTATGTCAAGCACATGGAC |
| | | TTACGCCTGAACAAGTGGTGGCAATTGCATCAAACGGCGGA |
| | | GGGAAGCAAGCCCTTGAAACCGTGCAAAGGCTCCTCCCGGT |
| | | GCTGTGCCAAGCCCATGGATTGACTCCAGAACAGGTGGTCG |
| | | CTATCGCATCAAACAATGGTGGAAAGCAAGCCCTCGAAACT |
| | | GTGCAGCGGCTCCTCCCTGTGTTGTGCCAGGCGCACGGTCT |
| | | GACTCCCGAGCAAGTCGTGGCAATCGCCTCGAACATCGGTG |
| | | GGAAGCAAGCTCTGGAGACTGTGCAGAGGCTCCTGCCGGTG |
| | | CTTTGTCAGGCCCACGGGCTTACTCCGGAACAAGTGGTGGC |
| | | CATCGCCTCCAACGGGGGGGGCAAACAGGCCTTGGAGACAG |
| | | TCCAGCGGCTCTTGCCGGTCTTGTGCCAAGCACACGGATTG |
| | | ACTCCAGAACAGGTGGTCGCAATCGCCAGCAATATCGGGGG |
| | | GAAGCAAGCCTTGGAAACCGTTCAGCGGCTACTCCCTGTGC |
| | | TGTGTCAAGCCCACGGTCTGACCCCAGAACAAGTGGTGGCT |
| | | ATCGCGTCGAACATTGGAGGCAAACAGGCCCTCGAGACTGT |
| | | CCAGAGACTCCTGCCAGTCCTCTGCCAGGCTCATGGGCTGA |
| | | CTCCTGAGCAAGTTGTGGCCATAGCATCCAACAACGGAGGA |
| | | AAGCAAGCTCTGGAAACCGTACAGAGACTTCTCCCGGTGTT |
| | | GTGTCAGGCCCACGGACTTACTCCGGAACAGGTTGTCGCCA |
| | | TCGCGTCCAACAACGGTGGCAGGCCTGCTCTGGAATCCATC |
| | | GTGGCACAGTTGTCAAGACCAGATCCTGCCCTGGCTGCGCT |
| | | CACCAACGACCACTTGGTGGCTCTGGCCTGCCTTGGCGGTC |
| | | GCCCTGCCTTGGATGCCGTGAAGAAGGGACTTCCTCACGCC |
| | | CCGGCACTGATTAAGCGCACCAATCGCAGGATCCCTGAACG |
| | | GACCTCGCACAGGGTC |
| 278 | TAL08 DBD | CATATGGTGGACCTGAGAACCCTTGGATACTCCCAGCAGCA |
| | RNA (from | GCAGGAGAAGATTAAGCCCAAGGTCCGGTCGACCGTCGCTC |
| | MR-32714) | AACACCACGAGGCCCTCGTGGGACATGGCTTCACCCACGCA |
| | | CACATTGTCGCGCTGTCCCAACACCCTGCCGCCCTTGGAAC |
| | | TGTGGCCGTCAAGTACCAGGATATGATCGCCGCTCTGCCTG |
| | | AAGCGACGCATGAAGCGATTGTGGGCGTGGGCAAGAGAGGA |
| | | GCAGGAGCCCGAGCACTGGAAGCCCTTCTGACCGTCGCCGG |
| | | CGAACTGAGGGGTCCACCTCTGCAACTGGATACCGGGCAGC |
| | | TGCTGAAGATCGCTAAGAGAGGCGGAGTGACCGCCGTCGAA |
| | | GCCGTCCATGCATGGCGCAACGCGCTGACTGGAGCACCGCT |
| | | GAACCTGACCCCCGAACAAGTCGTTGCCATTGCTAGTAACA |
| | | ACGGCGGCAAGCAGGCTCTGGAAACCGTCCAGCGCCTCCTT |
| | | CCTGTCCTGTGTCAAGCGCACGGGCTCACACCTGAACAAGT |
| | | AGTGGCCATCGCTTCCAACATTGGTGGCAAGCAGGCCCTGG |
| | | AAACTGTACAGCGGTTGTTGCCTGTCCTCTGCCAAGCGCAT |
| | | GGGCTTACCCCGGAGCAAGTGGTCGCTATCGCTTCTAACAA |
| | | CGGTGGGAAGCAGGCGCTGGAAACAGTGCAGAGACTGCTTC |
| | | CGGTGCTGTGCCAGGCACATGGCCTGACGCCGGAACAAGTG |
| | | GTGGCGATTGCATCTAACGGAGGCGGGAAGCAGGCGCTGGA |
| | | GACAGTGCAACGGCTGCTCCCTGTTCTATGCCAAGCTCATG |
| | | GACTCACCCCCGAGCAAGTCGTCGCCATTGCGTCCCACGAT |
| | | GGCGGAAAGCAGGCTTTGGAAACTGTGCAGCGCTTGCTGCC |
| | | TGTGCTCTGTCAGGCTCACGGACTCACCCCCGAGCAAGTCG |
| | | TAGCCATCGCCTCAAATATCGGAGGCAAGCAAGCGCTCGAG |
| | | ACTGTGCAGAGGCTCCTGCCTGTGTTATGTCAGGCACACGG |
| | | GCTGACCCCTGAACAAGTCGTGGCCATTGCCTCCAACGGTG |
| | | GCGGAAAACAGGCGCTCGAAACAGTCCAGAGGCTTTTGCCC |
| | | GTGCTGTGTCAAGCTCACGGCCTGACTCCGGAGCAAGTGGT |
| | | CGCCATTGCATCCCATGATGGCGGTAAACAAGCATTGGAAA |
| | | CGGTCCAACGCCTGCTGCCGGTCCTGTGCCAGGCCCATGGT |
| | | CTGACTCCCGAACAGGTCGTGGCTATCGCCTCCAACATCGG |
| | | TGGAAAACAAGCCCTGGAGACTGTACAGAGGCTGCTGCCCG |
| | | TGCTATGTCAAGCACATGGACTTACGCCTGAACAAGTGGTG |
| | | GCAATTGCATCACACGACGGAGGGAAGCAAGCCCTTGAAAC |
| | | CGTGCAACGACTTCTGCCCGTCCTGTGTCAGGCACACGGTC |
| | | TGACTCCCGAACAGGTGGTGGCCATCGCCAGCAATATCGGG |
| | | GGCAAGCAGGCGTTGGAAACTGTGCAAAGGCTCCTCCCGGT |
| | | GCTGTGCCAAGCCCATGGATTGACTCCAGAACAGGTGGTCG |
| | | CTATCGCATCACATGACGGTGGAAAGCAAGCCCTCGAAACT |
| | | GTGCAGCGGCTCCTCCCTGTGTTGTGCCAGGCGCACGGTCT |
| | | GACTCCCGAGCAAGTCGTGGCAATCGCCTCGAACGGAGGGG |
| | | GCAAACAAGCTCTCGAAACTGTTCAAAGACTGCTCCCCGTG |
| | | CTCTGTCAAGCTCACGGCCTCACTCCTGAACAGGTAGTCGC |
| | | GATTGCCTCCAACGGGGGGGGAAAGCAGGCCTTGGAGACAG |
| | | TCCAGCGGCTCTTGCCGGTCTTGTGCCAAGCACACGGATTG |
| | | ACTCCAGAACAGGTGGTCGCAATCGCCAGCCACGACGGGGG |
| | | GAAGCAAGCCTTGGAAACCGTTCAGCGGCTACTCCCTGTGC |
| | | TGTGTCAAGCCCACGGTCTGACCCCAGAACAAGTGGTGGCT |
| | | ATCGCGTCGCACGATGGAGGCAAACAGGCCCTCGAGACTGT |
| | | CCAGAGACTCCTGCCAGTCCTCTGCCAGGCTCATGGGCTGA |
| | | CTCCTGAGCAAGTTGTGGCCATAGCATCCAACGGCGGAGGA |
| | | AAGCAAGCTCTGGAAACCGTACAGAGACTTCTCCCGGTGTT |
| | | GTGTCAGGCCCACGGACTTACTCCGGAACAGGTTGTCGCCA |
| | | TCGCGTCCAACATAGGTGGCAGGCCTGCTCTGGAATCCATC |
| | | GTGGCACAGTTGTCAAGACCAGATCCTGCCCTGGCTGCGCT |
| | | CACCAACGACCACTTGGTGGCTCTGGCCTGCCTTGGCGGTC |
| | | GCCCTGCCTTGGATGCCGTGAAGAAGGGACTTCCTCACGCC |
| | | CCGGCACTGATTAAGCGCACCAATCGCAGGATCCCTGAACG |
| | | GACCTCGCACAGGGTC |
| 279 | TAL09 DBD | CATATGGTGGACCTGAGAACCCTTGGATACTCCCAGCAGCA |
| | RNA (from | GCAGGAGAAGATTAAGCCCAAGGTCCGGTCGACCGTCGCTC |
| | MR-32715) | AACACCACGAGGCCCTCGTGGGACATGGCTTCACCCACGCA |
| | | CACATTGTCGCGCTGTCCCAACACCCTGCCGCCCTTGGAAC |
| | | TGTGGCCGTCAAGTACCAGGATATGATCGCCGCTCTGCCTG |
| | | AAGCGACGCATGAAGCGATTGTGGGCGTGGGCAAGAGAGGA |
| | | GCAGGAGCCCGAGCACTGGAAGCCCTTCTGACCGTCGCCGG |
| | | CGAACTGAGGGGTCCACCTCTGCAACTGGATACCGGGCAGC |
| | | TGCTGAAGATCGCTAAGAGAGGCGGAGTGACCGCCGTCGAA |
| | | GCCGTCCATGCATGGCGCAACGCGCTGACTGGAGCACCGCT |
| | | GAACCTGACCCCCGAACAAGTCGTTGCCATTGCTAGTAACA |
| | | ACGGCGGCAAGCAGGCTCTGGAAACCGTCCAGCGCCTCCTT |
| | | CCTGTCCTGTGTCAAGCGCACGGGCTCACACCTGAACAAGT |
| | | AGTGGCCATCGCTTCCAACATTGGTGGCAAGCAGGCCCTGG |
| | | AAACTGTACAGCGGTTGTTGCCTGTCCTCTGCCAAGCGCAT |
| | | GGGCTTACCCCGGAGCAAGTGGTCGCTATCGCTTCTCATGA |
| | | TGGTGGGAAGCAGGCGCTGGAAACAGTGCAGAGACTGCTTC |
| | | CGGTGCTGTGCCAGGCACATGGCCTGACGCCGGAACAAGTG |
| | | GTGGCGATTGCATCTAACGGAGGCGGGAAGCAGGCGCTGGA |
| | | GACAGTGCAACGGCTGCTCCCTGTTCTATGCCAAGCTCATG |
| | | GACTCACCCCCGAGCAAGTCGTCGCCATTGCGTCCCACGAC |
| | | GGAGGCAAACAGGCACTGGAAACGGTGCAAAGACTGTTGCC |
| | | TGTACTGTGCCAGGCTCATGGCCTTACTCCCGAACAGGTGG |
| | | TGGCAATTGCGAGTAACATCGGCGGAAAGCAGGCTTTGGAA |
| | | ACTGTGCAGCGCTTGCTGCCTGTGCTCTGTCAGGCTCACGG |
| | | ACTCACCCCCGAGCAAGTCGTAGCCATCGCCTCAAATAACG |
| | | GAGGCAAGCAAGCGCTCGAGACTGTGCAGAGGCTCCTGCCT |
| | | GTGTTATGTCAGGCACACGGGCTGACCCCTGAACAAGTCGT |
| | | GGCCATTGCCTCCAACAACGGCGGAAAACAGGCGCTCGAAA |
| | | CAGTCCAGAGGCTTTTGCCCGTGCTGTGTCAAGCTCACGGC |
| | | CTGACTCCGGAGCAAGTGGTCGCCATTGCATCCAATGGCGG |
| | | CGGTAAACAAGCATTGGAAACGGTCCAACGCCTGCTGCCGG |
| | | TCCTGTGCCAGGCCCATGGTCTGACTCCCGAACAGGTCGTG |
| | | GCTATCGCCTCCAACGGAGGTGGAAAACAAGCCCTGGAGAC |
| | | TGTACAGAGGCTGCTGCCCGTGCTATGTCAAGCACATGGAC |
| | | TTACGCCTGAACAAGTGGTGGCAATTGCATCAAACGGGGGA |
| | | GGGAAGCAAGCCCTTGAAACCGTGCAAAGGCTCCTCCCGGT |
| | | GCTGTGCCAAGCCCATGGATTGACTCCAGAACAGGTGGTCG |
| | | CTATCGCATCAAACAACGGTGGAAAGCAAGCCCTCGAAACT |
| | | GTGCAGCGGCTCCTCCCTGTGTTGTGCCAGGCGCACGGTCT |
| | | GACTCCCGAGCAAGTCGTGGCAATCGCCTCGCACGATGGGG |
| | | GCAAACAAGCTCTCGAAACTGTTCAAAGACTGCTCCCCGTG |
| | | CTCTGTCAAGCTCACGGCCTCACTCCTGAACAGGTAGTCGC |
| | | GATTGCCTCCCATGACGGGGGAAAGCAGGCCTTGGAGACAG |
| | | TCCAGCGGCTCTTGCCGGTCTTGTGCCAAGCACACGGATTG |
| | | ACTCCAGAACAGGTGGTCGCAATCGCCAGCCATGATGGGGG |
| | | GAAGCAAGCCTTGGAAACCGTTCAGCGGCTACTCCCTGTGC |
| | | TGTGTCAAGCCCACGGTCTGACCCCAGAACAAGTGGTGGCT |
| | | ATCGCGTCGAACGGAGGAGGCAAACAGGCCCTCGAGACTGT |
| | | CCAGAGACTCCTGCCAGTCCTCTGCCAGGCTCATGGGCTGA |
| | | CTCCTGAGCAAGTTGTGGCCATAGCATCCAACAATGGAGGA |
| | | AAGCAAGCTCTGGAAACCGTACAGAGACTTCTCCCGGTGTT |
| | | GTGTCAGGCCCACGGACTTACTCCGGAACAGGTTGTCGCCA |
| | | TCGCGTCCAACATCGGTGGCAGGCCTGCTCTGGAATCCATC |
| | | GTGGCACAGTTGTCAAGACCAGATCCTGCCCTGGCTGCGCT |
| | | CACCAACGACCACTTGGTGGCTCTGGCCTGCCTTGGCGGTC |
| | | GCCCTGCCTTGGATGCCGTGAAGAAGGGACTTCCTCACGCC |
| | | CCGGCACTGATTAAGCGCACCAATCGCAGGATCCCTGAACG |
| | | GACCTCGCACAGGGTC |
| 280 | TAL10 DBD | CATATGGTGGACCTGAGAACCCTTGGATACTCCCAGCAGCA |
| | RNA (from | GCAGGAGAAGATTAAGCCCAAGGTCCGGTCGACCGTCGCTC |
| | MR-32913) | AACACCACGAGGCCCTCGTGGGACATGGCTTCACCCACGCA |
| | | CACATTGTCGCGCTGTCCCAACACCCTGCCGCCCTTGGAAC |
| | | TGTGGCCGTCAAGTACCAGGATATGATCGCCGCTCTGCCTG |
| | | AAGCGACGCATGAAGCGATTGTGGGCGTGGGCAAGAGAGGA |
| | | GCAGGAGCCCGAGCACTGGAAGCCCTTCTGACCGTCGCCGG |
| | | CGAACTGAGGGGTCCACCTCTGCAACTGGATACCGGGCAGC |
| | | TGCTGAAGATCGCTAAGAGAGGCGGAGTGACCGCCGTCGAA |
| | | GCCGTCCATGCATGGCGCAACGCGCTGACTGGAGCACCGCT |
| | | GAACCTCACCCCGGAACAGGTGGTGGCGATTGCTTCCCACG |
| | | ACGGTGGCAAGCAGGCCTTGGAAACCGTCCAACGGCTTTTG |
| | | CCGGTGCTTTGCCAGGCCCATGGACTGACCCCCGAACAAGT |
| | | CGTTGCCATTGCTAGTCACGACGGCGGCAAGCAGGCTCTGG |
| | | AAACCGTCCAGCGCCTCCTTCCTGTCCTGTGTCAAGCGCAC |
| | | GGGCTCACACCTGAACAAGTAGTGGCCATCGCTTCCAACGG |
| | | CGGTGGCAAGCAGGCCCTGGAAACTGTACAGCGGTTGTTGC |
| | | CTGTCCTCTGCCAAGCGCATGGGCTTACCCCGGAGCAAGTG |
| | | GTCGCTATCGCTTCTAATAATGGTGGGAAGCAGGCGCTGGA |
| | | AACAGTGCAGAGACTGCTTCCGGTGCTGTGCCAGGCACATG |
| | | GCCTGACGCCGGAACAAGTGGTGGCGATTGCATCTAACATT |
| | | GGCGGGAAGCAGGCGCTGGAGACAGTGCAACGGCTGCTCCC |
| | | TGTTCTATGCCAAGCTCATGGACTCACCCCCGAGCAAGTCG |
| | | TCGCCATTGCGTCCAACAACGGAGGCAAACAGGCACTGGAA |
| | | ACGGTGCAAAGACTGTTGCCTGTACTGTGCCAGGCTCATGG |
| | | CCTTACTCCCGAACAGGTGGTGGCAATTGCGAGTAACAACG |
| | | GTGGAAAGCAGGCGCTCGAAACTGTCCAGAGATTGCTGCCT |
| | | GTGCTGTGTCAGGCACACGGACTGACCCCAGAACAAGTGGT |
| | | GGCCATCGCGAGCCATGACGGCGGAAAGCAGGCTTTGGAAA |
| | | CTGTGCAGCGCTTGCTGCCTGTGCTCTGTCAGGCTCACGGA |
| | | CTCACCCCCGAGCAAGTCGTAGCCATCGCCTCAAATATCGG |
| | | AGGCAAGCAAGCGCTCGAGACTGTGCAGAGGCTCCTGCCTG |
| | | TGTTATGTCAGGCACACGGGCTGACCCCTGAACAAGTCGTG |
| | | GCCATTGCCTCCAACGGTGGCGGAAAACAGGCGCTCGAAAC |
| | | AGTCCAGAGGCTTTTGCCCGTGCTGTGTCAAGCTCACGGCC |
| | | TGACTCCGGAGCAAGTGGTCGCCATTGCATCCAATAACGGC |
| | | GGTAAACAAGCATTGGAAACGGTCCAACGCCTGCTGCCGGT |
| | | CCTGTGCCAGGCCCATGGTCTGACTCCCGAACAGGTCGTGG |
| | | CTATCGCCTCCAACGGAGGTGGAAAACAAGCCCTGGAGACT |
| | | GTACAGAGGCTGCTGCCCGTGCTATGTCAAGCACATGGACT |
| | | TACGCCTGAACAAGTGGTGGCAATTGCATCACACGACGGAG |
| | | GGAAGCAAGCCCTTGAAACCGTGCAAAGGCTCCTCCCGGTG |
| | | CTGTGCCAAGCCCATGGATTGACTCCAGAACAGGTGGTCGC |
| | | TATCGCATCAAACATCGGTGGAAAGCAAGCCCTCGAAACTG |
| | | TGCAGCGGCTCCTCCCTGTGTTGTGCCAGGCGCACGGTCTG |
| | | ACTCCCGAGCAAGTCGTGGCAATCGCCTCGAACAATGGGGG |
| | | CAAACAAGCTCTCGAAACTGTTCAAAGACTGCTCCCCGTGC |
| | | TCTGTCAAGCTCACGGCCTCACTCCTGAACAGGTAGTCGCG |
| | | ATTGCCTCCCATGACGGGGGAAAGCAGGCCTTGGAGACAGT |
| | | CCAGCGGCTCTTGCCGGTCTTGTGCCAAGCACACGGATTGA |
| | | CTCCAGAACAGGTGGTCGCAATCGCCAGCCATGATGGAGGA |
| | | AAGCAAGCTCTGGAAACCGTACAGAGACTTCTCCCGGTGTT |
| | | GTGTCAGGCCCACGGACTTACTCCGGAACAGGTTGTCGCCA |
| | | TCGCGTCCAACATCGGTGGCAGGCCTGCTCTGGAATCCATC |
| | | GTGGCACAGTTGTCAAGACCAGATCCTGCCCTGGCTGCGCT |
| | | CACCAACGACCACTTGGTGGCTCTGGCCTGCCTTGGCGGTC |
| | | GCCCTGCCTTGGATGCCGTGAAGAAGGGACTTCCTCACGCC |
| | | CCGGCACTGATTAAGCGCACCAATCGCAGGATCCCTGAACG |
| | | GACCTCGCACAGGGTC |
| 281 | TAL11 DBD | CATATGGTGGACCTGAGAACCCTTGGATACTCCCAGCAGCA |
| | RNA (from | GCAGGAGAAGATTAAGCCCAAGGTCCGGTCGACCGTCGCTC |
| | MR-32914) | AACACCACGAGGCCCTCGTGGGACATGGCTTCACCCACGCA |
| | | CACATTGTCGCGCTGTCCCAACACCCTGCCGCCCTTGGAAC |
| | | TGTGGCCGTCAAGTACCAGGATATGATCGCCGCTCTGCCTG |
| | | AAGCGACGCATGAAGCGATTGTGGGCGTGGGCAAGAGAGGA |
| | | GCAGGAGCCCGAGCACTGGAAGCCCTTCTGACCGTCGCCGG |
| | | CGAACTGAGGGGTCCACCTCTGCAACTGGATACCGGGCAGC |
| | | TGCTGAAGATCGCTAAGAGAGGCGGAGTGACCGCCGTCGAA |
| | | GCCGTCCATGCATGGCGCAACGCGCTGACTGGAGCACCGCT |
| | | GAACCTGACCCCCGAACAAGTCGTTGCCATTGCTAGTCATG |
| | | ACGGCGGCAAGCAGGCTCTGGAAACCGTCCAGCGCCTCCTT |
| | | CCTGTCCTGTGTCAAGCGCACGGGCTCACACCTGAACAAGT |
| | | AGTGGCCATCGCTTCCAACGGAGGTGGCAAGCAGGCCCTGG |
| | | AAACTGTACAGCGGTTGTTGCCTGTCCTCTGCCAAGCGCAT |
| | | GGGCTTACCCCGGAGCAAGTGGTCGCTATCGCTTCTAACAA |
| | | CGGAGGCAAGCAAGCGCTCGAGACTGTGCAGAGGCTCCTGC |
| | | CTGTGTTATGTCAGGCACACGGGCTGACCCCTGAACAAGTC |
| | | GTGGCCATTGCCTCCAACATCGGCGGAAAACAGGCGCTCGA |
| | | AACAGTCCAGAGGCTTTTGCCCGTGCTGTGTCAAGCTCACG |
| | | GCCTGACTCCGGAGCAAGTGGTCGCCATTGCATCCAACAAC |
| | | GGCGGTAAACAAGCATTGGAAACGGTCCAACGCCTGCTGCC |
| | | GGTCCTGTGCCAGGCCCATGGTCTGACTCCCGAACAGGTCG |
| | | TGGCTATCGCCTCCAACGGAGGTGGTAAGCAAGCACTGGAA |
| | | ACCGTGCAGCGGTTGCTCCCCGTGCTCTGTCAGGCTCACGG |
| | | GCTCACTCCAGAGCAGGTGGTGGCCATTGCCAGCCATGACG |
| | | GTGGAAAACAAGCCCTGGAGACTGTACAGAGGCTGCTGCCC |
| | | GTGCTATGTCAAGCACATGGACTTACGCCTGAACAAGTGGT |
| | | GGCAATTGCATCAAACATTGGAGGGAAGCAAGCCCTTGAAA |
| | | CCGTGCAACGACTTCTGCCCGTCCTGTGTCAGGCACACGGT |
| | | CTGACTCCCGAACAGGTGGTGGCCATCGCCAGCCACGACGG |
| | | GGGCAAGCAGGCGTTGGAAACTGTGCAAAGGCTCCTCCCGG |
| | | TGCTGTGCCAAGCCCATGGATTGACTCCAGAACAGGTGGTC |
| | | GCTATCGCATCACATGACGGTGGAAAGCAAGCCCTCGAAAC |
| | | TGTGCAGCGGCTCCTCCCTGTGTTGTGCCAGGCGCACGGTC |
| | | TGACTCCCGAGCAAGTCGTGGCAATCGCCTCGAACGGAGGG |
| | | GGCAAACAAGCTCTCGAAACTGTTCAAAGACTCTTGCCGGT |
| | | CTTGTGCCAAGCACACGGATTGACTCCAGAACAGGTGGTCG |
| | | CAATCGCCAGCCACGACGGCGGGAAGCAGGCACTCGAAACT |
| | | GTCCAGCGTCTGCTGCCTGTCCTCTGCCAAGCCCACGGCCT |
| | | CACTCCAGAACAGGTGGTCGCGATTGCAAGCAACGGCGGGG |
| | | GGAAGCAAGCCTTGGAAACCGTTCAGCGGCTACTCCCTGTG |
| | | CTGTGTCAAGCCCACGGTCTGACCCCAGAACAAGTGGTGGC |
| | | TATCGCGTCGCACGATGGAGGCAAACAGGCCCTCGAGACTG |
| | | TCCAGAGACTCCTGCCAGTCCTCTGCCAGGCTCATGGGCTG |
| | | ACTCCTGAGCAAGTTGTGGCCATAGCATCCAACGGCGGCGG |
| | | CAAGCAAGCCCTGGAGACTGTGCAACGGCTGTTGCCGGTCC |
| | | TGTGCCAGGCCCATGGTCTTACGCCGGAACAGGTCGTGGCC |
| | | ATCGCGTCAAACAACGGTGGCAAGCAGGCTCTGGAGACAGT |
| | | GCAACGCCTTCTGCCTGTGCTTTGCCAGGCCCACGGCTTGA |
| | | CCCCCGAGCAAGTGGTGGCCATCGCCTCGAATAACGGAGGA |
| | | AAGCAAGCTCTGGAAACCGTACAGAGACTTCTCCCGGTGTT |
| | | GTGTCAGGCCCACGGACTTACTCCGGAACAGGTTGTCGCCA |
| | | TCGCGTCCAACATAGGTGGCAGGCCTGCTCTGGAATCCATC |
| | | GTGGCACAGTTGTCAAGACCAGATCCTGCCCTGGCTGCGCT |
| | | CACCAACGACCACTTGGTGGCTCTGGCCTGCCTTGGCGGTC |
| | | GCCCTGCCTTGGATGCCGTGAAGAAGGGACTTCCTCACGCC |
| | | CCGGCACTGATTAAGCGCACCAATCGCAGGATCCCTGAACG |
| | | GACCTCGCACAGGGTC |
| 282 | TAL12 DBD | CATATGGTGGACCTGAGAACCCTTGGATACTCCCAGCAGCA |
| | RNA (from | GCAGGAGAAGATTAAGCCCAAGGTCCGGTCGACCGTCGCTC |
| | MR-32915) | AACACCACGAGGCCCTCGTGGGACATGGCTTCACCCACGCA |
| | | CACATTGTCGCGCTGTCCCAACACCCTGCCGCCCTTGGAAC |
| | | TGTGGCCGTCAAGTACCAGGATATGATCGCCGCTCTGCCTG |
| | | AAGCGACGCATGAAGCGATTGTGGGCGTGGGCAAGAGAGGA |
| | | GCAGGAGCCCGAGCACTGGAAGCCCTTCTGACCGTCGCCGG |
| | | CGAACTGAGGGGTCCACCTCTGCAACTGGATACCGGGCAGC |
| | | TGCTGAAGATCGCTAAGAGAGGCGGAGTGACCGCCGTCGAA |
| | | GCCGTCCATGCATGGCGCAACGCGCTGACTGGAGCACCGCT |
| | | GAACCTGACCCCCGAACAAGTCGTTGCCATTGCTAGTCATG |
| | | ACGGCGGCAAGCAGGCTCTGGAAACCGTCCAGCGCCTCCTT |
| | | CCTGTCCTGTGTCAAGCGCACGGGCTCACACCTGAACAAGT |
| | | AGTGGCCATCGCTTCCCATGATGGTGGCAAGCAGGCCCTGG |
| | | AAACTGTACAGCGGTTGTTGCCTGTCCTCTGCCAAGCGCAT |
| | | GGGCTTACCCCGGAGCAAGTGGTCGCTATCGCTTCTAACGG |
| | | CGGCGGCAAACAGGCACTGGAAACGGTGCAAAGACTGTTGC |
| | | CTGTACTGTGCCAGGCTCATGGCCTTACTCCCGAACAGGTG |
| | | GTGGCAATTGCGAGTAACAACGGCGGAAAGCAGGCTTTGGA |
| | | AACTGTGCAGCGCTTGCTGCCTGTGCTCTGTCAGGCTCACG |
| | | GACTCACCCCCGAGCAAGTCGTAGCCATCGCCTCAAATGGA |
| | | GGAGGCAAGCAAGCGCTCGAGACTGTGCAGAGGCTCCTGCC |
| | | TGTGTTATGTCAGGCACACGGGCTGACCCCTGAACAAGTCG |
| | | TGGCCATTGCCTCCAACAACGGCGGAAAACAGGCGCTCGAA |
| | | ACAGTCCAGAGGCTTTTGCCCGTGCTGTGTCAAGCTCACGG |
| | | CCTGACTCCGGAGCAAGTGGTCGCCATTGCATCCCACGACG |
| | | GCGGTAAACAAGCATTGGAAACGGTCCAACGCCTGCTGCCG |
| | | GTCCTGTGCCAGGCCCATGGGCTGACTCCGGAACAAGTCGT |
| | | CGCGATTGCGTCCAACATCGGGGGAAAGCAAGCACTCGAAA |
| | | CAGTGCAGCGGCTGCTGCCAGTCCTGTGCCAGGCACATGGC |
| | | CTGACCCCGGAGCAGGTGGTGGCCATCGCGAGCAACATTGG |
| | | TGGGAAGCAGGCACTGGAAACTGTCCAACGGCTGCTGCCGG |
| | | TGCTATGCCAGGCCCATGGTCTGACTCCCGAACAGGTCGTG |
| | | GCTATCGCCTCCAACGGAGGTGGAAAACAAGCCCTGGAGAC |
| | | TGTACAGAGGCTGCTGCCCGTGCTATGTCAAGCACATGGAC |
| | | TTACGCCTGAACAAGTGGTGGCAATTGCATCAAACAACGGA |
| | | GGGAAGCAAGCCCTTGAAACCGTGCAAAGGCTCCTCCCGGT |
| | | GCTGTGCCAAGCCCATGGATTGACTCCAGAACAGGTGGTCG |
| | | CTATCGCATCAAACGGAGGTGGAAAGCAAGCCCTCGAAACT |
| | | GTGCAGCGGCTCCTCCCTGTGTTGTGCCAGGCGCACGGTCT |
| | | GACTCCCGAGCAAGTCGTGGCAATCGCCTCGCACGACGGTG |
| | | GGAAGCAAGCTCTGGAGACTGTGCAGAGGCTCCTGCCGGTG |
| | | CTTTGTCAGGCCCACGGGCTTACTCCGGAACAAGTGGTGGC |
| | | CATCGCCTCCAACGGGGGGGGCAAACAGGCCTTGGAGACAG |
| | | TCCAGCGGCTCTTGCCGGTCTTGTGCCAAGCACACGGATTG |
| | | ACTCCAGAACAGGTGGTCGCAATCGCCAGCAATGGCGGGGG |
| | | GAAGCAAGCCTTGGAAACCGTTCAGCGGCTACTCCCTGTGC |
| | | TGTGTCAAGCCCACGGTCTGACCCCAGAACAAGTGGTGGCT |
| | | ATCGCGTCGAACGGGGGAGGCAAACAGGCCCTCGAGACTGT |
| | | CCAGAGACTCCTGCCAGTCCTCTGCCAGGCTCATGGGCTGA |
| | | CTCCTGAGCAAGTTGTGGCCATAGCATCCAACAACGGAGGA |
| | | AAGCAAGCTCTGGAAACCGTACAGAGACTTCTCCCGGTGTT |
| | | GTGTCAGGCCCACGGACTTACTCCGGAACAGGTTGTCGCCA |
| | | TCGCGTCCAACAACGGTGGCAGGCCTGCTCTGGAATCCATC |
| | | GTGGCACAGTTGTCAAGACCAGATCCTGCCCTGGCTGCGCT |
| | | CACCAACGACCACTTGGTGGCTCTGGCCTGCCTTGGCGGTC |
| | | GCCCTGCCTTGGATGCCGTGAAGAAGGGACTTCCTCACGCC |
| | | CCGGCACTGATTAAGCGCACCAATCGCAGGATCCCTGAACG |
| | | GACCTCGCACAGGGTC |
| 283 | TAL13 DBD | CATATGGTGGACCTGAGAACCCTTGGATACTCCCAGCAGCA |
| | RNA (from | GCAGGAGAAGATTAAGCCCAAGGTCCGGTCGACCGTCGCTC |
| | MR-32916) | AACACCACGAGGCCCTCGTGGGACATGGCTTCACCCACGCA |
| | | CACATTGTCGCGCTGTCCCAACACCCTGCCGCCCTTGGAAC |
| | | TGTGGCCGTCAAGTACCAGGATATGATCGCCGCTCTGCCTG |
| | | AAGCGACGCATGAAGCGATTGTGGGCGTGGGCAAGAGAGGA |
| | | GCAGGAGCCCGAGCACTGGAAGCCCTTCTGACCGTCGCCGG |
| | | CGAACTGAGGGGTCCACCTCTGCAACTGGATACCGGGCAGC |
| | | TGCTGAAGATCGCTAAGAGAGGCGGAGTGACCGCCGTCGAA |
| | | GCCGTCCATGCATGGCGCAACGCGCTGACTGGAGCACCGCT |
| | | GAACCTGACCCCCGAACAAGTCGTTGCCATTGCTAGTAACG |
| | | GTGGCGGCAAGCAGGCTCTGGAAACCGTCCAGCGCCTCCTT |
| | | CCTGTCCTGTGTCAAGCGCACGGGCTCACACCTGAACAAGT |
| | | AGTGGCCATCGCTTCCAACAATGGTGGCAAGCAGGCCCTGG |
| | | AAACTGTACAGCGGTTGTTGCCTGTCCTCTGCCAAGCGCAT |
| | | GGGCTTACCCCGGAGCAAGTGGTCGCTATCGCTTCTCATGA |
| | | TGGTGGGAAGCAGGCGCTGGAAACAGTGCAGAGACTGCTTC |
| | | CGGTGCTGTGCCAGGCACATGGCCTGACGCCGGAACAAGTG |
| | | GTGGCGATTGCATCTCACGATGGCGGGAAGCAGGCGCTGGA |
| | | GACAGTGCAACGGCTGCTCCCTGTTCTATGCCAAGCTCATG |
| | | GACTCACCCCCGAGCAAGTCGTCGCCATTGCGTCCAACGGA |
| | | GGAGGCAAACAGGCACTGGAAACGGTGCAAAGACTGTTGCC |
| | | TGTACTGTGCCAGGCTCATGGCCTTACTCCCGAACAGGTGG |
| | | TGGCAATTGCGAGTCACGACGGCGGAAAGCAGGCTTTGGAA |
| | | ACTGTGCAGCGCTTGCTGCCTGTGCTCTGTCAGGCTCACGG |
| | | ACTCACCCCCGAGCAAGTCGTAGCCATCGCCTCAAATATCG |
| | | GAGGCAAGCAAGCGCTCGAGACTGTGCAGAGGCTCCTGCCT |
| | | GTGTTATGTCAGGCACACGGGCTGACCCCTGAACAAGTCGT |
| | | GGCCATTGCCTCCAACAACGGCGGAAAACAGGCGCTCGAAA |
| | | CAGTCCAGAGGCTTTTGCCCGTGCTGTGTCAAGCTCACGGC |
| | | CTGACTCCGGAGCAAGTGGTCGCCATTGCATCCAACAATGG |
| | | CGGTAAACAAGCATTGGAAACGGTCCAACGCCTGCTGCCGG |
| | | TCCTGTGCCAGGCCCATGGTCTGACTCCCGAACAGGTCGTG |
| | | GCTATCGCCTCCAACATCGGAGGGAAGCAAGCCCTTGAAAC |
| | | CGTGCAAAGGCTCCTCCCGGTGCTGTGCCAAGCCCATGGAT |
| | | TGACTCCAGAACAGGTGGTCGCTATCGCATCAAACAACGGT |
| | | GGAAAGCAAGCCCTCGAAACTGTGCAGCGGCTCCTCCCTGT |
| | | GTTGTGCCAGGCGCACGGTCTGACTCCCGAGCAAGTCGTGG |
| | | CAATCGCCTCGAACGGAGGGGGCAAACAAGCTCTCGAAACT |
| | | GTTCAAAGACTGCTCCCCGTGCTCTGTCAAGCTCACGGCCT |
| | | CACTCCTGAACAGGTAGTCGCGATTGCCTCCCATGACGGGG |
| | | GAAAGCAGGCCTTGGAGACAGTCCAGCGGCTCTTGCCGGTC |
| | | TTGTGCCAAGCACACGGATTGACTCCAGAACAGGTGGTCGC |
| | | AATCGCCAGCAATATCGGCGGCAAGCAAGCCCTGGAGACTG |
| | | TTCAGAGGCTCTTGCCTGTGCTCTGCCAGGCCCATGGCTTG |
| | | ACACCTGAACAGGTGGTGGCCATCGCGAGCAACGGTGGGGG |
| | | GAAGCAAGCCTTGGAAACCGTTCAGCGGCTACTCCCTGTGC |
| | | TGTGTCAAGCCCACGGTCTGACCCCAGAACAAGTGGTGGCT |
| | | ATCGCGTCGAACAACGGAGGCAAACAGGCCCTCGAGACTGT |
| | | CCAGAGACTCCTGCCAGTCCTCTGCCAGGCTCATGGGCTGA |
| | | CTCCTGAGCAAGTTGTGGCCATAGCATCCAACAACGGAGGA |
| | | AAGCAAGCTCTGGAAACCGTACAGAGACTTCTCCCGGTGTT |
| | | GTGTCAGGCCCACGGACTTACTCCGGAACAGGTTGTCGCCA |
| | | TCGCGTCCAACAACGGTGGCAGGCCTGCTCTGGAATCCATC |
| | | GTGGCACAGTTGTCAAGACCAGATCCTGCCCTGGCTGCGCT |
| | | CACCAACGACCACTTGGTGGCTCTGGCCTGCCTTGGCGGTC |
| | | GCCCTGCCTTGGATGCCGTGAAGAAGGGACTTCCTCACGCC |
| | | CCGGCACTGATTAAGCGCACCAATCGCAGGATCCCTGAACG |
| | | GACCTCGCACAGGGTC |

TABLE 15
Exemplary TAL domain target sequences, e.g.,
for an expression repressor comprising an
effector moiety, e.g., KRAB

| SEQ | | |
|---|---|---|
| ID | | |
| NO | Description | Sequence |
| 292 | TAL06-KRAB Target | TACTGAAGCTCCACAATT |
| | Sequence (MR- | |
| | 32712) | |
| 293 | TAL07-KRAB Target | ACTCCGTATTTGATAAGG |
| | Sequence (MR- | |
| | 32713) | |
| 294 | TAL08-KRAB Target | GAGTCATCACACTTCCTA |
| | Sequence (MR- | |
| | 32714) | |
| 295 | TAL09-KRAB Target | GACTCAGGTTTGCCCTGA |
| | Sequence (MR- | |
| | 32715) | |
| 296 | TAL10-KRAB Target | CCTGAGGCATGTCAGCCA |
| | Sequence (MR- | |
| | 32913) | |
| 297 | TAL11-KRAB Target | CTGAGTCACCTCTCTGGA |
| | Sequence (MR- | |
| | 32914) | |
| 298 | TAL12-KRAB Target | CCTGTGCAATGTCTTTGG |
| | Sequence (MR- | |
| | 32915) | |
| 299 | TAL13-KRAB Target | TGCCTCAGGAGTCATGGG |
| | Sequence (MR- | |
| | 32916) | |

TABLE 15A
Exemplary TAL domain target sequences, e.g.,
for an expression repressor comprising an
effector moiety, e.g., KRAB, e.g., for
use in a murine model

| SEQ | | |
|---|---|---|
| ID | | |
| NO | Description | Sequence |
| 318 | Mouse TAL16-KRAB | GTCTAATCCTTGGGAGTG |
| | Target Sequence | GRCm38: chr5: |
| | (MR-32720) | 90891101-90891118 |
| 325 | Mouse TAL17-KRAB | CTGTAGTACACCAAGTGG |
| | Target Sequence | GRCm38: chr5: |
| | (MR-32721) | 90890903-90890920 |
| 332 | Mouse TAL17-KRAB | GTTAGGTCTCCACTGATG |
| | Target Sequence | GRCm38: chr5: |
| | (MR-32722) | 90903571-90903588 |
| 339 | Mouse TAL17-KRAB | CAGGGAATTTCCCTGGTC |
| | Target Sequence | GRCm38: chr5: |
| | (MR-32723) | 90903800-90903817 |

In some aspects, the disclosure provides an expression repressor comprising a first targeting moiety that binds to a target site, wherein the target site is within an IL-8 promoter (e.g., chr4:74606112-7460646, or within a site beginning 2 kb upstream and/or 2 kb downstream of chr4:74606112-7460646). In some embodiments, the expression repressor comprises a first effector moiety. In some embodiments, the expression repressor is capable of decreasing expression of IL-8.

In some aspects, the disclosure provides an expression repressor comprising a first targeting moiety that binds to a target site within genomic coordinates chr4:74606112-7460646 (based on hg19 human genome reference assembly). In some embodiments, the expression repressor comprises a first effector moiety. In some embodiments, the expression repressor is capable of decreasing expression of IL-8.

In some embodiments, the disclosure provides an expression repressor comprising a first targeting moiety, e.g., a TAL domain, wherein the targeting domain targets a site chosen from:
i) GRCh37: chr4:74606039-74606056;
ii) GRCh37: chr4:74606113-74606130;
iii) GRCh37: chr4:74606137-74606154;
iv) GRCh37: chr4:74606150-74606167;
v) GRCh37: chr4:74591882-74591899;
vi) GRCh37: chr4:74591923-74591940;
vii) GRCh37: chr4:74591897-74591914; and
viii) GRCh37: chr4:74591873-74591890.

In some embodiments, the disclosure provides an expression repressor comprising a first targeting moiety, e.g., a TAL domain, wherein the targeting domain targets a mouse site chosen from:
i) GRCm38: chr5:90891101-90891118;
ii) GRCm38: chr5:90890903-90890920;
iii) GRCm38: chr5:90903571-90903588; or
iv) GRCm38: chr5:90903800-90903817.

In some embodiments, the TAL domain binds within 500, 300, 200, 100, or 50 nucleotides upstream or downstream of a target site chosen from: i) GRCm38: chr5:90891101-90891118; ii) GRCm38: chr5:90890903-90890920; iii) GRCm38: chr5:90903571-90903588; and iv) GRCm38: chr5:90903800-90903817.

In some embodiments, a targeting moiety is or comprises a DNA-binding domain from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort, et al. (1997) Nucleic Acids Res. 25:3379-3388; Dujon, et al. (1989) Gene 82:115-118; Perler, et al. (1994) Nucleic Acids Res. 22:1125-1127; Jasin (1996) Trends Genet. 12:224-228; Gimble, et al. (1996) J. Mol. Biol. 263:163-180; Argast, et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier, et al. (2002) Molec. Cell 10:895-905; Epinat, et al. (2003) Nucleic Acids Res. 31:2952-2962; Ashworth, et al. (2006) Nature 441:656-659; Paques, et al. (2007) Current Gene Therapy 7:49-66; U.S. Patent Publication No. 2007/0117128.

In some embodiments, a targeting moiety comprises a nucleic acid. In some embodiments, a nucleic acid that may be included in a targeting moiety, may be or comprise DNA, RNA, and/or an artificial or synthetic nucleic acid or nucleic acid analog or mimic. For example, in some embodiments, a nucleic acid may be or include one or more of genomic DNA (gDNA), complementary DNA (cDNA), a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA as described herein and/or that targets an expression product of a particular gene associated with a targeted genomic complex as described herein), etc. In some embodiments, a nucleic acid may include one or more residues that is not a naturally occurring DNA or RNA residue, may include one or more linkages that is/are not phosphodiester bonds (e.g., that may be, for example, phosphorothioate bonds, etc), and/or may include one or more modifications such as, for example, a 2′O modification such as 2′-OMeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

A nucleic acid suitable for use in an expression repressor or a site-specific disrupting agent, e.g., in a targeting moiety, may include, but is not limited to, DNA, RNA, modified oligonucleotides (e.g., chemical modifications, such as modifications that alter backbone linkages, sugar molecules, and/or nucleic acid bases), and artificial nucleic acids. In some embodiments, a nucleic acid includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules.

In some embodiments, a targeting moiety comprises a nucleic acid with a length from about 15-200, 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 215-190, 20-190, 30-190, 40-190, 50-190, 60-190, 70-190, 80-190, 90-190, 100-190, 110-190, 120-190, 130-190, 140-190, 150-190, 160-190, 170-190, 180-190, 15-180, 20-180, 30-180, 40-180, 50-180, 60-180, 70-180, 80-180, 90-180, 100-180, 110-180, 120-180, 130-180, 140-180, 150-180, 160-180, 170-180, 15-170, 20-170, 30-170, 40-170, 50-170, 60-170, 70-170, 80-170, 90-170, 100-170, 110-170, 120-170, 130-170, 140-170, 150-170, 160-170, 15-160, 20-160, 30-160, 40-160, 50-160, 60-160, 70-160, 80-160, 90-160, 100-160, 110-160, 120-160, 130-160, 140-160, 150-160, 215-150, 20-150, 30-150, 40-150, 50-150, 60-150, 70-150, 80-150, 90-150, 100-150, 110-150, 120-150, 130-150, 140-150, 15-140, 20-140, 30-140, 40-140, 50-140, 60-140, 70-140, 80-140, 90-140, 100-140, 110-140, 120-140, 130-140, 15-130, 20-130, 30-130, 40-130, 50-130, 60-130, 70-130, 80-130, 90-130, 100-130, 110-130, 120-130, 215-120, 20-120, 30-120, 40-120, 50-120, 60-120, 70-120, 80-120, 90-120, 100-120, 110-120, 15-110, 20-110, 30-110, 40-110, 50-110, 60-110, 70-110, 80-110, 90-110, 100-110, 15-100, 20-100, 30-100, 40-100, 50-100, 60-100, 70-100, 80-100, 90-100, 15-90, 20-90, 30-90, 40-90, 50-90, 60-90, 70-90, 80-90, 15-80, 20-80, 30-80, 40-80, 50-80, 60-80, 70-80, 15-70, 20-70, 30-70, 40-70, 50-70, 60-70, 15-60, 20-60, 30-60, 40-60, 50-60, 15-50, 20-50, 30-50, 40-50, 15-40, 20-40, 30-40, 15-30, 20-30, or 15-20 nucleotides, or any range therebetween.

### Effector Moieties

An expression repressor or a site-specific disrupting agent of the present disclosure may comprise one or more effector moieties. An effector moiety has one or more functionalities that, when used as part of a site-specific disrupting agent described herein, modulate, e.g., decrease, expression of a target plurality of genes in a cell. In some embodiments, an effector moiety physically or sterically blocks an anchor sequence, e.g., such that binding of a genomic complex component (e.g., a nucleating polypeptide) to the anchor sequence is inhibited (e.g., prevented). In some embodiments, an effector moiety destabilizes the interaction of a genomic complex component (e.g., nucleating polypeptide) with an anchor sequence, e.g., by altering (e.g., decreasing) the affinity and/or avidity at which the genomic complex component binds the anchor sequence. For example, an effector moiety may recruit a factor that inhibits formation of or destabilizes a genomic complex, e.g., ASMC, or it may inhibit recruitment of a factor (e.g., a genomic complex component or transcription factor) necessary for formation or maintenance of a genomic complex (e.g., ASMC). In some embodiments, an effector moiety has epigenetic modification functionality in that it modulates the epigenetic landscape of the target site (e.g., the E1 cRE, or a sequence proximal thereto, or an anchor sequence or a sequence proximal to the anchor sequence). e.g., by promoting (e.g., catalyzing) application or removal of one or more epigenetic modifications to the DNA or a histone associated thereto, to decrease expression of a target plurality of genes. In some embodiments, an effector moiety has genetic modification functionality, e.g., it introduces an alteration (e.g., an insertion, deletion, or substitution) to a target site (e.g., an E1 cRE or a sequence proximal thereto, or an anchor sequence or a sequence proximal thereto) or a sequence proximal thereto.

In some embodiments, an effector moiety comprises a CRISPR/Cas molecule, a TAL effector molecule, a Zn finger molecule, a tetR domain, or a meganuclease. In some embodiments, an effector moiety has genetic modification functionality, e.g., a CRISPR/Cas molecule, a TAL effector molecule, a Zn finger molecule with endonuclease activity capable of making a genetic alteration in a method described herein.

In some embodiments, an effector moiety comprises a histone modifying functionality, e.g., a histone methyltransferase, histone demethylase, or histone deacetylase activity. In some embodiments, a histone methyltransferase functionality comprises H3K9 targeting methyltransferase activity. In some embodiments, a histone methyltransferase functionality comprises H3K56 targeting methyltransferase activity. In some embodiments, a histone methyltransferase functionality comprises H3K27 targeting methyltransferase activity. In some embodiments, a histone methyltransferase or demethylase functionality transfers one, two, or three methyl groups. In some embodiments, a histone demethylase functionality comprises H3K4 targeting demethylase activity. In some embodiments, an effector moiety comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof. In some embodiments, an effector moiety comprises a protein chosen from KDM1A (i.e., LSD1), KDM1B (i.e., LSD2), KDM2A, KDM2B, KDM5A, KDM5B, KDM5C, KDM5D, KDM4B, N066, or a functional variant or fragment of any thereof. In some embodiments, an effector moiety comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.

In some embodiments, an effector moiety comprises a DNA modifying functionality, e.g., a DNA methyltransferase. In some embodiments, an effector moiety comprises a protein chosen from MQ1, DNMT1, DNMT3A1, DNMT3A2, DNMT3B1, DNMT3B2, DNMT3B3, DNMT3B4. DNMT3B5, DNMT3B6, DNMT3L, or a functional variant or fragment of any thereof.

In some embodiments, an effector moiety comprises a transcription repressor. In some embodiments the transcription repressor blocks recruitment of a factor that stimulates or promotes transcription, e.g., of the target gene. In some embodiments, the transcription repressor recruits a factor that inhibits transcription, e.g., of the target gene. In some embodiments, an effector moiety, e.g., transcription repressor, is or comprises a protein chosen from KRAB, MeCP2, HP1, RBBP4, REST, FOG1, SUZ12, or a functional variant or fragment of any thereof.

In some embodiments, an effector moiety comprises a protein having a functionality described herein. In some embodiments, an effector moiety comprises a protein selected from:
KRAB (e.g., as according to NP_056209.2 or the protein encoded by NM_015394.5);
a SET domain (e.g., the SET domain of:
SETDB1 (e.g., as according to NP_001353347.1 or the protein encoded by NM_001366418.1);
EZH2 (e.g., as according to NP-004447.2 or NP_001190176.1 or the protein encoded by NM_004456.5 or NM_001203247.2);
G9A (e.g., as according to NP_001350618.1 or the protein encoded by NM_001363689.1); or
SUV39H1 (e.g., as according to NP_003164.1 or the protein encoded by NM_003173.4));
histone demethylase LSD1 (e.g., as according to NP_055828.2 or the protein encoded by NM_015013.4);
FOG1 (e.g., the N-terminal residues of FOG1) (e.g., as according to NP_722520.2 or the protein
encoded by NM_153813.3); or KAP1 (e.g., as according to NP_005753.1 or the protein encoded by NM_005762.3);
a functional fragment or variant of any thereof, or
a polypeptide with a sequence that has at least 80, 85, 90, 91, 92, 93, 94, 95, 96, 97, 98, or 99% identity to any of the above-referenced sequences. In some embodiments, an effector moiety comprises a protein selected from:
DNMT3A (e.g., human DNMT3A) (e.g., as according to NP_072046.2
or the protein encoded by NM_022552.4);
DNMT3B (e.g., as according to NP_008823.1
or the protein encoded by NM_006892.4);
DNMT3L (e.g., as according to NP_787063.1
or the protein encoded by NM_175867.3);
DNMT3A/3L complex,
bacterial MQ1 (e.g., as according to CAA35058.1 obtained from strain ATCC 33825 or Uniprot ID P15840.3);
a functional fragment of any thereof, or
a polypeptide with a sequence that has at least 80, 85, 90, 91, 92, 93, 94, 95, 96, 97, 98, or 99% identity to any of the above-referenced sequences. In some embodiments, an effector moiety comprises a mature bacterial MQ1 (e.g., as according to CAA35058.1 obtained from strain ATCC 33825 or Uniprot ID P15840.3

An exemplary effector moiety may include, but is not limited to: ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3A, DNMT3B, DNMT3L), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdb1), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDM1A and lysine-specific histone demethylase 1 (LSD1), helicases such as DHX9, deacetylases (e.g., sirtuin 1, 2, 3, 4, 5, 6, or 7), kinases, phosphatases, DNA-intercalating agents such as ethidium bromide, SYBR green, and proflavine, efflux pump inhibitors such as peptidomimetics like phenylalanine arginyl β-naphthylamide or quinoline derivatives, nuclear receptor activators and inhibitors, proteasome inhibitors, competitive inhibitors for enzymes such as those involved in lysosomal storage diseases, protein synthesis inhibitors, nucleases (e.g., Cpf1, Cas9, zinc finger nuclease), fusions of one or more thereof (e.g., dCas9-DNMT, dCas9-APOBEC, dCas9-UG1), and specific domains from proteins, such as a KRAB domain.

In some embodiments, a candidate domain may be determined to be suitable for use as an effector moiety by methods known to those of skill in the art. For example, a candidate effector moiety may be tested by assaying whether, when the candidate effector moiety is present in the nucleus of a cell and appropriately localized (e.g., to a target gene or genomic regulatory element (e.g., transcription control element) operably linked to said target gene, e.g., via a targeting moiety), the candidate effector moiety decreases expression of the target gene in the cell, e.g., decreases the level of RNA transcript encoded by the target gene (e.g., as measured by RNASeq or Northern blot) or decreases the level of protein encoded by the target gene (e.g., as measured by ELISA).

In some embodiments, an expression repressor comprises an effector moiety that does not bind (e.g., does not detectably bind) to another copy of the effector moiety, e.g., the effector moiety is monomeric and does not associate into multimers. In some embodiments, an expression repressor comprises an effector moiety that associates with a further copy of the effector moiety into a multimer, e.g., dimers, trimers, tetramers, or further. In some embodiments, an expression repressor comprises a plurality of effector moieties, wherein each effector moiety does not detectably bind, e.g., does not bind, to another effector moiety. In some embodiments, an effector moiety for use in the compositions and methods described herein is functional in a monomeric, e.g., non-dimeric, state.

In some embodiments, a site-specific disrupting agent comprises an effector moiety that does not bind (e.g., does not detectably bind) to another copy of the effector moiety, e.g., the effector moiety is monomeric and does not associate into multimers. In some embodiments, a site-specific disrupting agent comprises an effector moiety that associates with a further copy of the effector moiety into a multimer, e.g., dimers, trimers, tetramers, or further. In some embodiments, a site-specific disrupting agent comprises a plurality of effector moieties, wherein each effector moiety does not detectably bind, e.g., does not bind, to another effector moiety. In some embodiments, an effector moiety for use in the compositions and methods described herein is functional in a monomeric, e.g., non-dimeric, state.

In some embodiments, an effector moiety of an expression repressor or a site-specific disrupting agent comprises an epigenetic modifying moiety, e.g., that modulates the two-dimensional structure of chromatin (i.e., that modulate structure of chromatin in a way that would alter its two-dimensional representation).

Epigenetic modifying moieties useful in methods and compositions of the present disclosure include agents that affect epigenetic markers, e.g., DNA methylation, histone methylation, histone acetylation, histone sumoylation, histone phosphorylation, and RNA-associated silencing. Exemplary epigenetic enzymes that can be targeted to a genomic sequence element as described herein include DNA methylases (e.g., DNMT3a, DNMT3b, DNMTL, MQ1), DNA demethylation (e.g., the TET family), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012):1-18.

In some embodiments, an expression repressor or site-specific disrupting agent, e.g., comprising an epigenetic modifying moiety, useful herein comprises or is a construct described in Koferle et al. Genome Medicine 7.59 (2015):1-3 incorporated herein by reference. For example, in some embodiments, a site-specific disrupting agent comprises or is a construct found in Table 1 of Koferle et al., e.g., histone deacetylase, histone methyltransferase, DNA demethylation, or H3K4 and/or H3K9 histone demethylase described in Table 1 (e.g., dCas9-p300, TALE-TET1, ZF-DNMT3A, or TALE-LSD1).

### Additional Moieties

An expression repressor may further comprise one or more additional moieties (e.g., in addition to one or more targeting moieties and one or more effector moieties). In some embodiments, an additional moiety is selected from a tagging or monitoring moiety, a cleavable moiety (e.g., a cleavable moiety positioned between a targeting moiety and an effector moiety or at the N- or C-terminal end of a polypeptide), a small molecule, a membrane translocating polypeptide, or a pharmacoagent moiety.

A site-specific disrupting agent may further comprise one or more additional moieties (e.g., in addition to one or more targeting moieties and one or more effector moieties). In some embodiments, an additional moiety is selected from a tagging or monitoring moiety, a cleavable moiety (e.g., a cleavable moiety positioned between a targeting moiety and an effector moiety or at the N- or C-terminal end of a polypeptide), a small molecule, a membrane translocating polypeptide, or a pharmacoagent moiety.

### Exemplary Expression Repressors

The following exemplary expression repressors are presented for illustration purposes only and are not intended to be limiting.

In some embodiments, an expression repressor comprises a targeting moiety (e.g., comprising dCas9, e.g., an S. pyogenes dCas9), and an effector moiety comprising KRAB, e.g., a KRAB domain. In some embodiments, the expression repressor is encoded by the nucleic acid sequence of SEQ ID NOs: 204 (e.g., a plasmid encoding the expression repressor) and/or 205 (e.g., a nucleic acid (e.g., mRNA) encoding the expression repressor). In some embodiments, a nucleic acid described herein comprises a nucleic acid sequence of SEQ ID NO: 204 or 205 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the targeting moiety is encoded by the nucleic acid sequence of SEQ ID NO. 8 and the effector moiety is encoded by the nucleic acid sequence of SEQ ID NO. 14.

### Sp-dCas9-KRAB (PL-27687) Plasmid DNA Sequence:

(SEQ ID NO: 204)
TCGCGCGTTTCGGTGATGACGGTGAAAACCTCTGACACATGCAGC
TCCCGGAGACGGTCACAGCTTGTCTGTAAGCGGATGCCGGGAGCA
GACAAGCCCGTCAGGGCGCGTCAGCGGGTGTTGGCGGGTGTCGGG
GCTGGCTTAACTATGCGGCATCAGAGCAGATTGTACTGAGAGTGC
ACCATATGCGGTGTGAAATACCGCACAGATGCGTAAGGAGAAAAT
ACCGCATCAGGCGCCATTCGCCATTCAGGCTGCGCAACTGTTGGG
AAGGGCGATCGGTGCGGGCCTCTTCGCTATTACGCCAGCTGGCGA
AAGGGGGATGTGCTGCAAGGCGATTAAGTTGGGTAACGCCAGGGT
TTTCCCAGTCACGACGTTGTAAAACGACGGCCAGTGAATTGACGC
GTATTGGGATGGTACCTAATACGACTCACTATAAGGAAATAAGAG
AGAAAAGAAGAGTAAGAAGAAATATAAGAGCCACCATGGCCCCCA
AGAAGAAGCGGAAGGTGGGCATCCACGGCGTGCCCGCCGCCGACA
AGAAGTACAGCATCGGCCTGGCCATCGGCACCAACAGCGTGGGCT
GGGCCGTGATCACCGACGAGTACAAGGTGCCCAGCAAGAAGTTCA
AGGTGCTGGGCAACACCGACCGGCACAGCATCAAGAAGAACCTGA
TCGGCGCCCTGCTGTTCGACAGCGGCGAGACCGCCGAGGCCACCC
GGCTGAAGCGGACCGCCCGGCGGCGGTACACCCGGCGGAAGAACC
GGATCTGCTACCTGCAGGAGATCTTCAGCAACGAGATGGCCAAGG
TGGACGACAGCTTCTTCCACCGGCTGGAGGAGAGCTTCCTGGTGG
AGGAGGACAAGAAGCACGAGCGGCACCCCATCTTCGGCAACATCG
TGGACGAGGTGGCCTACCACGAGAAGTACCCCACCATCTACCACC
TGCGGAAGAAGCTGGTGGACAGCACCGACAAGGCCGACCTGCGGC
TGATCTACCTGGCCCTGGCCCACATGATCAAGTTCCGGGGCCACT
TCCTGATCGAGGGCGACCTGAACCCCGACAACAGCGACGTGGACA
AGCTGTTCATCCAGCTGGTGCAGACCTACAACCAGCTGTTCGAGG
AGAACCCCATCAACGCCAGCGGCGTGGACGCCAAGGCCATCCTGA
GCGCCCGGCTGAGCAAGAGCCGGCGGCTGGAGAACCTGATCGCCC
AGCTGCCCGGCGAGAAGAAGAACGGCCTGTTCGGCAACCTGATCG
CCCTGAGCCTGGGCCTGACCCCCAACTTCAAGAGCAACTTCGACC
TGGCCGAGGACGCCAAGCTGCAGCTGAGCAAGGACACCTACGACG
ACGACCTGGACAACCTGCTGGCCCAGATCGGCGACCAGTACGCCG
ACCTGTTCCTGGCCGCCAAGAACCTGAGCGACGCCATCCTGCTGA
GCGACATCCTGCGGGTGAACACCGAGATCACCAAGGCCCCCCTGA
GCGCCAGCATGATCAAGCGGTACGACGAGCACCACCAGGACCTGA
CCCTGCTGAAGGCCCTGGTGCGGCAGCAGCTGCCCGAGAAGTACA
AGGAGATCTTCTTCGACCAGAGCAAGAACGGCTACGCCGGCTACA
TCGACGGCGGCGCCAGCCAGGAGGAGTTCTACAAGTTCATCAAGC
CCATCCTGGAGAAGATGGACGGCACCGAGGAGCTGCTGGTGAAGC
TGAACCGGGAGGACCTGCTGCGGAAGCAGCGGACCTTCGACAACG
GCAGCATCCCCCACCAGATCCACCTGGGCGAGCTGCACGCCATCC
TGCGGCGGCAGGAGGACTTCTACCCCTTCCTGAAGGACAACCGGG
AGAAGATCGAGAAGATCCTGACCTTCCGGATCCCCTACTACGTGG
GCCCCCTGGCCCGGGGCAACAGCCGGTTCGCCTGGATGACCCGGa
aatccGAGGAGACCATCACCCCCTGGAACTTCGAGGAGGTGGTGG
ACAAGGGCGCCAGCGCCCAGAGCTTCATCGAGCGGATGACCAACT
TCGACAAGAACCTGCCCAACGAGAAGGTGCTGCCCAAGCACAGCC
TGCTGTACGAGTACTTCACCGTGTACAACGAGCTGACCAAGGTGA
AGTACGTGACCGAGGGCATGCGGAAGCCCGCCTTCCTGAGCGGCG
AGCAGAAGAAGGCCATCGTGGACCTGCTGTTCAAGACCAACCGGA
AGGTGACCGTGAAGCAGCTGAAGGAGGACTACTTCAAGAAGATCG
AGTGCTTCGACAGCGTGGAGATCAGCGGCGTGGAGGACCGGTTCA
ACGCCAGCCTGGGCACCTACCACGACCTGCTGAAGATCATCAAGG
ACAAGGACTTCCTGGACAACGAGGAGAACGAGGACATCCTGGAGG
ACATCGTGCTGACCCTGACCCTGTTCGAGGACCGGGAGATGATCG
AGGAGCGGCTGAAaACCTACGCCCACCTGTTCGACGACAAGGTGA
TGAAGCAGCTGAAGCGGCGGCGGTACACCGGCTGGGGCCGGCTGA
GCCGGAAGCTGATCAACGGCATCCGGGACAAGCAGAGCGGCAAGA
CCATCCTGGACTTCCTGaaatccGACGGCTTCGCCAACCGGAACT
TCATGCAGCTGATCCACGACGACAGCCTGACCTTCAAGGAGGACA
TCCAGAAGGCCCAGGTGAGCGGCCAGGGCGACAGCCTGCACGAGC
ACATCGCCAACCTGGCCGGCAGCCCCGCCATCAAGAAGGGCATCC
TGCAGACCGTGAAGGTGGTGGACGAGCTGGTGAAGGTGATGGGCC
GGCACAAGCCCGAGAACATCGTGATCGAGATGGCCCGGGAGAACC
AGACCACCCAGAAGGGCCAGAAGAACAGCCGGGAGCGGATGAAGC
GGATCGAGGAGGGCATCAAGGAGCTGGGCAGCCAGATCCTGAAGG
AGCACCCCGTGGAGAACACCCAGCTGCAGAACGAGAAGCTGTACC
TGTACTACCTGCAGAACGGCCGGGACATGTACGTGGACCAGGAGC
TGGACATCAACCGGCTGAGCGACTACGACGTGGCCGCCATCGTGC
CCCAGAGCTTCCTGAAGGACGACAGCATCGACAACAAGGTGCTGA
CCCGGAGCGACAAGGCCCGGGGCAAGAGCGACAACGTGCCCAGCG
AGGAGGTGGTGAAGAAGATGAAGAACTACTGGCGGCAGCTGCTGA
ACGCCAAGCTGATCACCCAGCGGAAGTTCGACAACCTGACCAAGG
CCGAGCGGGGCGGCCTGAGCGAGCTGGACAAGGCCGGCTTCATCA
AGCGGCAGCTGGTGGAGACCCGGCAGATCACCAAGCACGTGGCCC
AGATCCTGGACAGCCGGATGAACACCAAGTACGACGAGAACGACA
AGCTGATCCGGGAGGTGAAGGTGATCACCCTGaaatccAAGCTGG
TGAGCGACTTCCGGAAGGACTTCCAGTTCTACAAGGTGCGGGAGA
TCAACAACTACCACCACGCCCACGACGCCTACCTGAACGCCGTGG
TGGGCACCGCCCTGATCAAGAAGTACCCCAAGCTGGAGAGCGAGT
TCGTGTACGGCGACTACAAGGTGTACGACGTGCGGAAGATGATCG
CCAAGAGCGAGCAGGAGATCGGCAAGGCCACCGCCAAGTACTTCT
TCTACAGCAACATCATGAACTTCTTCAAGACCGAGATCACCCTGG
CCAACGGCGAGATCCGGAAGCGGCCCCTGATCGAGACCAACGGCG
AGACCGGCGAGATCGTGTGGGACAAGGGCCGGGACTTCGCCACCG
TGCGGAAGGTGCTGAGCATGCCCCAGGTGAACATCGTGAAGAAaA
CCGAGGTGCAGACCGGCGGCTTCAGCAAGGAGAGCATCCTGCCCA
AGCGGAACAGCGACAAGCTGATCGCCCGGAAGAAGGACTGGGACC
CCAAGAAGTACGGCGGCTTCGACAGCCCCACCGTGGCCTACAGCG
TGCTGGTGGTGGCCAAGGTGGAGAAGGGCAAGAGCAAGAAGCTGa
aatccGTGAAGGAGCTGCTGGGCATCACCATCATGGAGCGGAGCA
GCTTCGAGAAGAACCCCATCGACTTCCTGGAGGCCAAGGGCTACA
AGGAGGTGAAGAAGGACCTGATCATCAAGCTGCCCAAGTACAGCC
TGTTCGAGCTGGAGAACGGCCGGAAGCGGATGCTGGCCAGCGCCG
GCGAGCTGCAGAAGGGCAACGAGCTGGCCCTGCCCAGCAAGTACG
TGAACTTCCTGTACCTGGCCAGCCACTACGAGAAGCTGAAGGGCA
GCCCCGAGGACAACGAGCAGAAGCAGCTGTTCGTGGAGCAGCACA
AGCACTACCTGGACGAGATCATCGAGCAGATCAGCGAGTTCAGCA
AGCGGGTGATCCTGGCCGACGCCAACCTGGACAAGGTGCTGAGCG
CCTACAACAAGCACCGGGACAAGCCCATCCGGGAGCAGGCCGAGA
ACATCATCCACCTGTTCACCCTGACCAACCTGGGCGCCCCCGCCG
CCTTCAAGTACTTCGACACCACCATCGACCGGAAGCGGTACACCA
GCACCAAGGAGGTGCTGGACGCCACCCTGATCCACCAGAGCATCA
CCGGCCTGTACGAGACCCGGATCGACCTGAGCCAGCTGGGCGGCG
ACAAGCGGCCCGCCGCCACCAAGAAGGCCGGCCAGGCCAAGAAGA
AGAAGGCCAGCGACGCCAAGAGCCTGACCGCCTGGAGCCGGACCC
TGGTGACCTTCAAGGACGTGTTCGTGGACTTCACCCGGGAGGAGT
GGAAGCTGCTGGACACCGCCCAGCAGATCCTGTACCGGAACGTGA
TGCTGGAGAACTACAAGAACCTGGTGAGCCTGGGCTACCAGCTGA
CCAAGCCCGACGTGATCCTGCGGCTGGAGAAGGGCGAGGAGCCCT
GGCTGGTGGAGCGGGAGATCCACCAGGAGACCCACCCCGACAGCG
AGACCGCCTTCGAGATCAAGAGCAGCGTGAGCGGCGGCAAGCGGC
CCGCCGCCACCAAGAAGGCCGGCCAGGCCAAGAAGAAGAAGGGCA
GCTACCCCTACGACGTGCCCGACTACGCCTGAGcggccgcttaat
taagctgccttctgcggggcttgccttctggccatgcccttcttc
tctcccttgcacctgtacctcttggtctttgaataaagcctgagt
aggaagtctagaaaaaaaaaaaaaaaaaaaaaaaaaaaaaaaaaa
aaaaaaaaaaaaaaaaaaaattgtcttcttcatcgcctgcagATC
CCAATGGCGCGCCGAGCTTGGCTCGAGCATGGTCATAGCTGTTTC
CTGTGTGAAATTGTTATCCGCTCACAATTCCACACAACATACGAG
CCGGAAGCATAAAGTGTAAAGCCTGGGGTGCCTAATGAGTGAGCT
AACTCACATTAATTGCGTTGCGCTCACTGCCCGCTTTCCAGTCGG
GAAACCTGTCGTGCCAGCTGCATTAATGAATCGGCCAACGCGCGG
GGAGAGGCGGTTTGCGTATTGGGCGCTCTTCCGCTTCCTCGCTCA
CTGACTCGCTGCGCTCGGTCGTTCGGCTGCGGCGAGCGGTATCAG
CTCACTCAAAGGCGGTAATACGGTTATCCACAGAATCAGGGGATA
ACGCAGGAAAGAACATGTGAGCAAAAGGCCAGCAAAAGGCCAGGA
ACCGTAAAAAGGCCGCGTTGCTGGCGTTTTTCCATAGGCTCCGCC
CCCCTGACGAGCATCACAAAAATCGACGCTCAAGTCAGAGGTGGC
GAAACCCGACAGGACTATAAAGATACCAGGCGTTTCCCCCTGGAA
GCTCCCTCGTGCGCTCTCCTGTTCCGACCCTGCCGCTTACCGGAT
ACCTGTCCGCCTTTCTCCCTTCGGGAAGCGTGGCGCTTTCTCATA
GCTCACGCTGTAGGTATCTCAGTTCGGTGTAGGTCGTTCGCTCCA
AGCTGGGCTGTGTGCACGAACCCCCCGTTCAGCCCGACCGCTGCG
CCTTATCCGGTAACTATCGTCTTGAGTCCAACCCGGTAAGACACG
ACTTATCGCCACTGGCAGCAGCCACTGGTAACAGGATTAGCAGAG
CGAGGTATGTAGGCGGTGCTACAGAGTTCTTGAAGTGGTGGCCTA
ACTACGGCTACACTAGAAGAACAGTATTTGGTATCTGCGCTCTGC
TGAAGCCAGTTACCTTCGGAAAAAGAGTTGGTAGCTCTTGATCCG
GCAAACAAACCACCGCTGGTAGCGGTGGTTTTTTTGTTTGCAAGC
AGCAGATTACGCGCAGAAAAAAAGGATCTCAAGAAGATCCTTTGA
TCTTTTCTACGGGGTCTGACGCTCAGTGGAACGAAAACTCACGTT
AAGGGATTTTGGTCATGAGATTATCAAAAAGGATCTTCACCTAGA
TCCTTTTAAATTAAAAATGAAGTTTTAAATCAATCTAAAGTATAT
ATGAGTAAACTTGGTCTGACAGTTAGAAAAACTCATCGAGCATCA
AATGAAACTGCAATTTATTCATATCAGGATTATCAATACCATATT
TTTGAAAAAGCCGTTTCTGTAATGAAGGAGAAAACTCACCGAGGC
AGTTCCATAGGATGGCAAGATCCTGGTATCGGTCTGCGATTCCGA
CTCGTCCAACATCAATACAACCTATTAATTTCCCCTCGTCAAAAA
TAAGGTTATCAAGTGAGAAATCACCATGAGTGACGACTGAATCCG
GTGAGAATGGCAAAAGTTTATGCATTTCTTTCCAGACTTGTTCAA
CAGGCCAGCCATTACGCTCGTCATCAAAATCACTCGCATCAACCA
AACCGTTATTCATTCGTGATTGCGCCTGAGCGAGACGAAATACGC
GATCGCTGTTAAAAGGACAATTACAAACAGGAATCGAATGCAACC
GGCGCAGGAACACTGCCAGCGCATCAACAATATTTTCACCTGAAT
CAGGATATTCTTCTAATACCTGGAATGCTGTTTTCCCAGGGATCG
CAGTGGTGAGTAACCATGCATCATCAGGAGTACGGATAAAATGCT
TGATGGTCGGAAGAGGCATAAATTCCGTCAGCCAGTTTAGTCTGA
CCATCTCATCTGTAACATCATTGGCAACGCTACCTTTGCCATGTT
TCAGAAACAACTCTGGCGCATCGGGCTTCCCATACAATCGATAGA
TTGTCGCACCTGATTGCCCGACATTATCGCGAGCCCATTTATACC
CATATAAATCAGCATCCATGTTGGAATTTAATCGCGGCCTAGAGC
AAGACGTTTCCCGTTGAATATGGCTCATACTCTTCCTTTTTCAAT
ATTATTGAAGCATTTATCAGGGTTATTGTCTCATGAGCGGATACA
TATTTGAATGTATTTAGAAAAATAAACAAATAGGGGTTCCGCGCA
CATTTCCCCGAAAAGTGCCACCTGACGTCTAAGAAACCATTATTA
TCATGACATTAACCTATAAAAATAGGCGTATCACGAGGCCCTTTC
GTC

Sp-dCas9-KRAB (MR-28122) Expressed mRNA Sequence:

(SEQ ID NO: 205)
AGGAAATAAGAGAGAAAAGAAGAGTAAGAAGAAATATAAGAGCCA
CCATGGCCCCCAAGAAGAAGCGGAAGGTGGGCATCCACGGCGTGC
CCGCCGCCGACAAGAAGTACAGCATCGGCCTGGCCATCGGCACCA
ACAGCGTGGGCTGGGCCGTGATCACCGACGAGTACAAGGTGCCCA
GCAAGAAGTTCAAGGTGCTGGGCAACACCGACCGGCACAGCATCA
AGAAGAACCTGATCGGCGCCCTGCTGTTCGACAGCGGCGAGACCG
CCGAGGCCACCCGGCTGAAGCGGACCGCCCGGCGGCGGTACACCC
GGCGGAAGAACCGGATCTGCTACCTGCAGGAGATCTTCAGCAACG
AGATGGCCAAGGTGGACGACAGCTTCTTCCACCGGCTGGAGGAGA
GCTTCCTGGTGGAGGAGGACAAGAAGCACGAGCGGCACCCCATCT
TCGGCAACATCGTGGACGAGGTGGCCTACCACGAGAAGTACCCCA
CCATCTACCACCTGCGGAAGAAGCTGGTGGACAGCACCGACAAGG
CCGACCTGCGGCTGATCTACCTGGCCCTGGCCCACATGATCAAGT
TCCGGGGCCACTTCCTGATCGAGGGCGACCTGAACCCCGACAACA
GCGACGTGGACAAGCTGTTCATCCAGCTGGTGCAGACCTACAACC
AGCTGTTCGAGGAGAACCCCATCAACGCCAGCGGCGTGGACGCCA
AGGCCATCCTGAGCGCCCGGCTGAGCAAGAGCCGGCGGCTGGAGA
ACCTGATCGCCCAGCTGCCCGGCGAGAAGAAGAACGGCCTGTTCG
GCAACCTGATCGCCCTGAGCCTGGGCCTGACCCCCAACTTCAAGA
GCAACTTCGACCTGGCCGAGGACGCCAAGCTGCAGCTGAGCAAGG
ACACCTACGACGACGACCTGGACAACCTGCTGGCCCAGATCGGCG
ACCAGTACGCCGACCTGTTCCTGGCCGCCAAGAACCTGAGCGACG
CCATCCTGCTGAGCGACATCCTGCGGGTGAACACCGAGATCACCA
AGGCCCCCCTGAGCGCCAGCATGATCAAGCGGTACGACGAGCACC
ACCAGGACCTGACCCTGCTGAAGGCCCTGGTGCGGCAGCAGCTGC
CCGAGAAGTACAAGGAGATCTTCTTCGACCAGAGCAAGAACGGCT
ACGCCGGCTACATCGACGGCGGCGCCAGCCAGGAGGAGTTCTACA
AGTTCATCAAGCCCATCCTGGAGAAGATGGACGGCACCGAGGAGC
TGCTGGTGAAGCTGAACCGGGAGGACCTGCTGCGGAAGCAGCGGA
CCTTCGACAACGGCAGCATCCCCCACCAGATCCACCTGGGCGAGC
TGCACGCCATCCTGCGGCGGCAGGAGGACTTCTACCCCTTCCTGA
AGGACAACCGGGAGAAGATCGAGAAGATCCTGACCTTCCGGATCC
CCTACTACGTGGGCCCCCTGGCCCGGGGCAACAGCCGGTTCGCCT
GGATGACCCGGaaatccGAGGAGACCATCACCCCCTGGAACTTCG
AGGAGGTGGTGGACAAGGGCGCCAGCGCCCAGAGCTTCATCGAGC
GGATGACCAACTTCGACAAGAACCTGCCCAACGAGAAGGTGCTGC
CCAAGCACAGCCTGCTGTACGAGTACTTCACCGTGTACAACGAGC
TGACCAAGGTGAAGTACGTGACCGAGGGCATGCGGAAGCCCGCCT
TCCTGAGCGGCGAGCAGAAGAAGGCCATCGTGGACCTGCTGTTCA
AGACCAACCGGAAGGTGACCGTGAAGCAGCTGAAGGAGGACTACT
TCAAGAAGATCGAGTGCTTCGACAGCGTGGAGATCAGCGGCGTGG
AGGACCGGTTCAACGCCAGCCTGGGCACCTACCACGACCTGCTGA
AGATCATCAAGGACAAGGACTTCCTGGACAACGAGGAGAACGAGG
ACATCCTGGAGGACATCGTGCTGACCCTGACCCTGTTCGAGGACC
GGGAGATGATCGAGGAGCGGCTGAAaACCTACGCCCACCTGTTCG
ACGACAAGGTGATGAAGCAGCTGAAGCGGCGGCGGTACACCGGCT
GGGGCCGGCTGAGCCGGAAGCTGATCAACGGCATCCGGGACAAGC
AGAGCGGCAAGACCATCCTGGACTTCCTGaaatccGACGGCTTCG
CCAACCGGAACTTCATGCAGCTGATCCACGACGACAGCCTGACCT
TCAAGGAGGACATCCAGAAGGCCCAGGTGAGCGGCCAGGGCGACA
GCCTGCACGAGCACATCGCCAACCTGGCCGGCAGCCCCGCCATCA
AGAAGGGCATCCTGCAGACCGTGAAGGTGGTGGACGAGCTGGTGA
AGGTGATGGGCCGGCACAAGCCCGAGAACATCGTGATCGAGATGG
CCCGGGAGAACCAGACCACCCAGAAGGGCCAGAAGAACAGCCGGG
AGCGGATGAAGCGGATCGAGGAGGGCATCAAGGAGCTGGGCAGCC
AGATCCTGAAGGAGCACCCCGTGGAGAACACCCAGCTGCAGAACG
AGAAGCTGTACCTGTACTACCTGCAGAACGGCCGGGACATGTACG
TGGACCAGGAGCTGGACATCAACCGGCTGAGCGACTACGACGTGG
CCGCCATCGTGCCCCAGAGCTTCCTGAAGGACGACAGCATCGACA
ACAAGGTGCTGACCCGGAGCGACAAGGCCCGGGGCAAGAGCGACA
ACGTGCCCAGCGAGGAGGTGGTGAAGAAGATGAAGAACTACTGGC
GGCAGCTGCTGAACGCCAAGCTGATCACCCAGCGGAAGTTCGACA
ACCTGACCAAGGCCGAGCGGGGCGGCCTGAGCGAGCTGGACAAGG
CCGGCTTCATCAAGCGGCAGCTGGTGGAGACCCGGCAGATCACCA
AGCACGTGGCCCAGATCCTGGACAGCCGGATGAACACCAAGTACG
ACGAGAACGACAAGCTGATCCGGGAGGTGAAGGTGATCACCCTGa
aatccAAGCTGGTGAGCGACTTCCGGAAGGACTTCCAGTTCTACA
AGGTGCGGGAGATCAACAACTACCACCACGCCCACGACGCCTACC
TGAACGCCGTGGTGGGCACCGCCCTGATCAAGAAGTACCCCAAGC
TGGAGAGCGAGTTCGTGTACGGCGACTACAAGGTGTACGACGTGC
GGAAGATGATCGCCAAGAGCGAGCAGGAGATCGGCAAGGCCACCG
CCAAGTACTTCTTCTACAGCAACATCATGAACTTCTTCAAGACCG
AGATCACCCTGGCCAACGGCGAGATCCGGAAGCGGCCCCTGATCG
AGACCAACGGCGAGACCGGCGAGATCGTGTGGGACAAGGGCCGGG
ACTTCGCCACCGTGCGGAAGGTGCTGAGCATGCCCCAGGTGAACA
TCGTGAAGAAaACCGAGGTGCAGACCGGCGGCTTCAGCAAGGAGA
GCATCCTGCCCAAGCGGAACAGCGACAAGCTGATCGCCCGGAAGA
AGGACTGGGACCCCAAGAAGTACGGCGGCTTCGACAGCCCCACCG
TGGCCTACAGCGTGCTGGTGGTGGCCAAGGTGGAGAAGGGCAAGA
GCAAGAAGCTGaaatccGTGAAGGAGCTGCTGGGCATCACCATCA
TGGAGCGGAGCAGCTTCGAGAAGAACCCCATCGACTTCCTGGAGG
CCAAGGGCTACAAGGAGGTGAAGAAGGACCTGATCATCAAGCTGC
CCAAGTACAGCCTGTTCGAGCTGGAGAACGGCCGGAAGCGGATGC
TGGCCAGCGCCGGCGAGCTGCAGAAGGGCAACGAGCTGGCCCTGC
CCAGCAAGTACGTGAACTTCCTGTACCTGGCCAGCCACTACGAGA
AGCTGAAGGGCAGCCCCGAGGACAACGAGCAGAAGCAGCTGTTCG
TGGAGCAGCACAAGCACTACCTGGACGAGATCATCGAGCAGATCA
GCGAGTTCAGCAAGCGGGTGATCCTGGCCGACGCCAACCTGGACA
AGGTGCTGAGCGCCTACAACAAGCACCGGGACAAGCCCATCCGGG
AGCAGGCCGAGAACATCATCCACCTGTTCACCCTGACCAACCTGG
GCGCCCCCGCCGCCTTCAAGTACTTCGACACCACCATCGACCGGA
AGCGGTACACCAGCACCAAGGAGGTGCTGGACGCCACCCTGATCC
ACCAGAGCATCACCGGCCTGTACGAGACCCGGATCGACCTGAGCC
AGCTGGGCGGCGACAAGCGGCCCGCCGCCACCAAGAAGGCCGGCC
AGGCCAAGAAGAAGAAGGCCAGCGACGCCAAGAGCCTGACCGCCT
GGAGCCGGACCCTGGTGACCTTCAAGGACGTGTTCGTGGACTTCA
CCCGGGAGGAGTGGAAGCTGCTGGACACCGCCCAGCAGATCCTGT
ACCGGAACGTGATGCTGGAGAACTACAAGAACCTGGTGAGCCTGG
GCTACCAGCTGACCAAGCCCGACGTGATCCTGCGGCTGGAGAAGG
GCGAGGAGCCCTGGCTGGTGGAGCGGGAGATCCACCAGGAGACCC
ACCCCGACAGCGAGACCGCCTTCGAGATCAAGAGCAGCGTGAGCG
GCGGCAAGCGGCCCGCCGCCACCAAGAAGGCCGGCCAGGCCAAGA
AGAAGAAGGGCAGCTACCCCTACGACGTGCCCGACTACGCCTGAG
cggccgcttaattaagctgccttctgcggggcttgccttctggcc
atgcccttcttctctcccttgcacctgtacctcttggtctttgaa
taaagcctgagtaggaagtctagaaaaaaaaaaaaaaaaaaaaaa
aaaaaaaaaaaaaaaaaaaaaaaaaaaaaaaaaaaaaaaaaaaaa
aaaaaaaaaaaaa

In some embodiments, an expression repressor comprises the amino acid sequence of SEQ ID NOs: 206 or 75. In some embodiments, a nucleic acid described herein comprises a nucleic acid sequence of SEQ ID NO: 206 or 75 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### Sp-dCas9-KRAB Protein Sequence:

(SEQ ID NO: 206)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDKRPAATKKAGQ
AKKKKASDAKSLTAWSRTLVTFKDVFVDFTREEWKLLDTAQQILY
RNVMLENYKNLVSLGYQLTKPDVILRLEKGEEPWLVEREIHQETH
PDSETAFEIKSSVSGGKRPAATKKAGQAKKKKGSYPYDVPDYA

Sp-dCas9-KRAB Protein Sequence without HA Tag:

(SEQ ID NO: 75)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDKRPAATKKAGQ
AKKKKASDAKSLTAWSRTLVTFKDVFVDFTREEWKLLDTAQQILY
RNVMLENYKNLVSLGYQLTKPDVILRLEKGEEPWLVEREIHQETH
PDSETAFEIKSSVSGGKRPAATKKAGQAKKKKGS

In some embodiments, the expression repressor comprises a targeting moiety (e.g., comprising dCas9, e.g., an S. pyogenes dCas9 or a functional variant or mutant thereof; e.g., Cas9m4), and an effector moiety comprising MQ1, e.g., bacterial MQ1. In some embodiments, expression repressor is encoded by the nucleic acid sequence of SEQ ID NOs: 207 (e.g., a nucleic acid (e.g., mRNA) encoding the site-specific disrupting agent). In some embodiments, a nucleic acid described herein comprises a nucleic acid sequence of SEQ ID NO: 207 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the targeting moiety is encoded by the nucleic acid sequence of SEQ ID NO: 8 and/or the effector moiety is encoded by the nucleic acid sequence of SEQ ID NO: 10.

dCas9-MQ1 mRNA Sequence (MR28125)

(SEQ ID NO: 207)
AAGGAAAUAAGAGAGAAAAGAAGAGUAAGAAGAAAUAUAAGAGCC
ACCAUGGCCCCCAAGAAGAAGCGGAAGGUGGGCAUCCACGGCGUG
CCCGCCGCCGACAAGAAGUACAGCAUCGGCCUGGCCAUCGGCACC
AACAGCGUGGGCUGGGCCGUGAUCACCGACGAGUACAAGGUGCCC
AGCAAGAAGUUCAAGGUGCUGGGCAACACCGACCGGCACAGCAUC
AAGAAGAACCUGAUCGGCGCCCUGCUGUUCGACAGCGGCGAGACC
GCCGAGGCCACCCGGCUGAAGCGGACCGCCCGGCGGCGGUACACC
CGGCGGAAGAACCGGAUCUGCUACCUGCAGGAGAUCUUCAGCAAC
GAGAUGGCCAAGGUGGACGACAGCUUCUUCCACCGGCUGGAGGAG
AGCUUCCUGGUGGAGGAGGACAAGAAGCACGAGCGGCACCCCAUC
UUCGGCAACAUCGUGGACGAGGUGGCCUACCACGAGAAGUACCCC
ACCAUCUACCACCUGCGGAAGAAGCUGGUGGACAGCACCGACAAG
GCCGACCUGCGGCUGAUCUACCUGGCCCUGGCCCACAUGAUCAAG
UUCCGGGGCCACUUCCUGAUCGAGGGCGACCUGAACCCCGACAAC
AGCGACGUGGACAAGCUGUUCAUCCAGCUGGUGCAGACCUACAAC
CAGCUGUUCGAGGAGAACCCCAUCAACGCCAGCGGCGUGGACGCC
AAGGCCAUCCUGAGCGCCCGGCUGAGCAAGAGCCGGCGGCUGGAG
AACCUGAUCGCCCAGCUGCCCGGCGAGAAGAAGAACGGCCUGUUC
GGCAACCUGAUCGCCCUGAGCCUGGGCCUGACCCCCAACUUCAAG
AGCAACUUCGACCUGGCCGAGGACGCCAAGCUGCAGCUGAGCAAG
GACACCUACGACGACGACCUGGACAACCUGCUGGCCCAGAUCGGC
GACCAGUACGCCGACCUGUUCCUGGCCGCCAAGAACCUGAGCGAC
GCCAUCCUGCUGAGCGACAUCCUGCGGGUGAACACCGAGAUCACC
AAGGCCCCCCUGAGCGCCAGCAUGAUCAAGCGGUACGACGAGCAC
CACCAGGACCUGACCCUGCUGAAGGCCCUGGUGCGGCAGCAGCUG
CCCGAGAAGUACAAGGAGAUCUUCUUCGACCAGAGCAAGAACGGC
UACGCCGGCUACAUCGACGGCGGCGCCAGCCAGGAGGAGUUCUAC
AAGUUCAUCAAGCCCAUCCUGGAGAAGAUGGACGGCACCGAGGAG
CUGCUGGUGAAGCUGAACCGGGAGGACCUGCUGCGGAAGCAGCGG
ACCUUCGACAACGGCAGCAUCCCCCACCAGAUCCACCUGGGCGAG
CUGCACGCCAUCCUGCGGCGGCAGGAGGACUUCUACCCCUUCCUG
AAGGACAACCGGGAGAAGAUCGAGAAGAUCCUGACCUUCCGGAUC
CCCUACUACGUGGGCCCCCUGGCCCGGGGCAACAGCCGGUUCGCC
UGGAUGACCCGGAAAUCCGAGGAGACCAUCACCCCCUGGAACUUC
GAGGAGGUGGUGGACAAGGGCGCCAGCGCCCAGAGCUUCAUCGAG
CGGAUGACCAACUUCGACAAGAACCUGCCCAACGAGAAGGUGCUG
CCCAAGCACAGCCUGCUGUACGAGUACUUCACCGUGUACAACGAG
CUGACCAAGGUGAAGUACGUGACCGAGGGCAUGCGGAAGCCCGCC
UUCCUGAGCGGCGAGCAGAAGAAGGCCAUCGUGGACCUGCUGUUC
AAGACCAACCGGAAGGUGACCGUGAAGCAGCUGAAGGAGGACUAC
UUCAAGAAGAUCGAGUGCUUCGACAGCGUGGAGAUCAGCGGCGUG
GAGGACCGGUUCAACGCCAGCCUGGGCACCUACCACGACCUGCUG
AAGAUCAUCAAGGACAAGGACUUCCUGGACAACGAGGAGAACGAG
GACAUCCUGGAGGACAUCGUGCUGACCCUGACCCUGUUCGAGGAC
CGGGAGAUGAUCGAGGAGCGGCUGAAAACCUACGCCCACCUGUUC
GACGACAAGGUGAUGAAGCAGCUGAAGCGGCGGCGGUACACCGGC
UGGGGCCGGCUGAGCCGGAAGCUGAUCAACGGCAUCCGGGACAAG
CAGAGCGGCAAGACCAUCCUGGACUUCCUGAAAUCCGACGGCUUC
GCCAACCGGAACUUCAUGCAGCUGAUCCACGACGACAGCCUGACC
UUCAAGGAGGACAUCCAGAAGGCCCAGGUGAGCGGCCAGGGCGAC
AGCCUGCACGAGCACAUCGCCAACCUGGCCGGCAGCCCCGCCAUC
AAGAAGGGCAUCCUGCAGACCGUGAAGGUGGUGGACGAGCUGGUG
AAGGUGAUGGGCCGGCACAAGCCCGAGAACAUCGUGAUCGAGAUG
GCCCGGGAGAACCAGACCACCCAGAAGGGCCAGAAGAACAGCCGG
GAGCGGAUGAAGCGGAUCGAGGAGGGCAUCAAGGAGCUGGGCAGC
CAGAUCCUGAAGGAGCACCCCGUGGAGAACACCCAGCUGCAGAAC
GAGAAGCUGUACCUGUACUACCUGCAGAACGGCCGGGACAUGUAC
GUGGACCAGGAGCUGGACAUCAACCGGCUGAGCGACUACGACGUG
GCCGCCAUCGUGCCCCAGAGCUUCCUGAAGGACGACAGCAUCGAC
AACAAGGUGCUGACCCGGAGCGACAAGGCCCGGGGCAAGAGCGAC
AACGUGCCCAGCGAGGAGGUGGUGAAGAAGAUGAAGAACUACUGG
CGGCAGCUGCUGAACGCCAAGCUGAUCACCCAGCGGAAGUUCGAC
AACCUGACCAAGGCCGAGCGGGGCGGCCUGAGCGAGCUGGACAAG
GCCGGCUUCAUCAAGCGGCAGCUGGUGGAGACCCGGCAGAUCACC
AAGCACGUGGCCCAGAUCCUGGACAGCCGGAUGAACACCAAGUAC
GACGAGAACGACAAGCUGAUCCGGGAGGUGAAGGUGAUCACCCUG
AAAUCCAAGCUGGUGAGCGACUUCCGGAAGGACUUCCAGUUCUAC
AAGGUGCGGGAGAUCAACAACUACCACCACGCCCACGACGCCUAC
CUGAACGCCGUGGUGGGCACCGCCCUGAUCAAGAAGUACCCCAAG
CUGGAGAGCGAGUUCGUGUACGGCGACUACAAGGUGUACGACGUG
CGGAAGAUGAUCGCCAAGAGCGAGCAGGAGAUCGGCAAGGCCACC
GCCAAGUACUUCUUCUACAGCAACAUCAUGAACUUCUUCAAGACC
GAGAUCACCCUGGCCAACGGCGAGAUCCGGAAGCGGCCCCUGAUC
GAGACCAACGGCGAGACCGGCGAGAUCGUGUGGGACAAGGGCCGG
GACUUCGCCACCGUGCGGAAGGUGCUGAGCAUGCCCCAGGUGAAC
AUCGUGAAGAAAACCGAGGUGCAGACCGGCGGCUUCAGCAAGGAG
AGCAUCCUGCCCAAGCGGAACAGCGACAAGCUGAUCGCCCGGAAG
AAGGACUGGGACCCCAAGAAGUACGGCGGCUUCGACAGCCCCACC
GUGGCCUACAGCGUGCUGGUGGUGGCCAAGGUGGAGAAGGGCAAG
AGCAAGAAGCUGAAAUCCGUGAAGGAGCUGCUGGGCAUCACCAUC
AUGGAGCGGAGCAGCUUCGAGAAGAACCCCAUCGACUUCCUGGAG
GCCAAGGGCUACAAGGAGGUGAAGAAGGACCUGAUCAUCAAGCUG
CCCAAGUACAGCCUGUUCGAGCUGGAGAACGGCCGGAAGCGGAUG
CUGGCCAGCGCCGGCGAGCUGCAGAAGGGCAACGAGCUGGCCCUG
CCCAGCAAGUACGUGAACUUCCUGUACCUGGCCAGCCACUACGAG
AAGCUGAAGGGCAGCCCCGAGGACAACGAGCAGAAGCAGCUGUUC
GUGGAGCAGCACAAGCACUACCUGGACGAGAUCAUCGAGCAGAUC
AGCGAGUUCAGCAAGCGGGUGAUCCUGGCCGACGCCAACCUGGAC
AAGGUGCUGAGCGCCUACAACAAGCACCGGGACAAGCCCAUCCGG
GAGCAGGCCGAGAACAUCAUCCACCUGUUCACCCUGACCAACCUG
GGCGCCCCCGCCGCCUUCAAGUACUUCGACACCACCAUCGACCGG
AAGCGGUACACCAGCACCAAGGAGGUGCUGGACGCCACCCUGAUC
CACCAGAGCAUCACCGGCCUGUACGAGACCCGGAUCGACCUGAGC
CAGCUGGGCGGCGACAAGCGGCCCGCCGCCACCAAGAAGGCCGGC
CAGGCCAAGAAGAAGAAGGCCCGGGACAGCAAGGUGGAGAACAAG
ACCAAGAAGCUGCGGGUGUUCGAGGCCUUCGCCGGCAUCGGCGCC
CAGCGGAAGGCCCUGGAGAAGGUGCGGAAGGACGAGUACGAGAUC
GUGGGCCUGGCCGAGUGGUACGUGCCCGCCAUCGUGAUGUACCAG
GCCAUCCACAACAACUUCCACACCAAGCUGGAGUACAAGAGCGUG
AGCCGGGAGGAGAUGAUCGACUACCUGGAGAACAAGACCCUGAGC
UGGAACAGCAAGAACCCCGUGAGCAACGGCUACUGGAAGCGGAAG
AAGGACGACGAGCUGAAGAUCAUCUACAACGCCAUCAAGCUGAGC
GAGAAGGAGGGCAACAUCUUCGACAUCCGGGACCUGUACAAGCGG
ACCCUGAAGAACAUCGACCUGCUGACCUACAGCUUCCCCUGCCAG
GACCUGAGCCAGCAGGGCAUCCAGAAGGGCAUGAAGCGGGGCAGC
GGCACCCGGAGCGGCCUGCUGUGGGAGAUCGAGCGGGCCCUGGAC
AGCACCGAGAAGAACGACCUGCCCAAGUACCUGCUGAUGGAGAAC
GUGGGCGCCCUGCUGCACAAGAAGAACGAGGAGGAGCUGAACCAG
UGGAAGCAGAAGCUGGAGAGCCUGGGCUACCAGAACAGCAUCGAG
GUGCUGAACGCCGCCGACUUCGGCAGCAGCCAGGCCCGGCGGCGG
GUGUUCAUGAUCAGCACCCUGAACGAGUUCGUGGAGCUGCCCAAG
GGCGACAAGAAGCCCAAGAGCAUCAAGAAGGUGCUGAACAAGAUC
GUGAGCGAGAAGGACAUCCUGAACAACCUGCUGAAGUACAACCUG
ACCGAGUUCAAGAAAACCAAGAGCAACAUCAACAAGGCCAGCCUG
AUCGGCUACAGCAAGUUCAACAGCGAGGGCUACGUGUACGACCCC
GAGUUCACCGGCCCCACCCUGACCGCCAGCGGCGCCAACAGCCGG
AUCAAGAUCAAGGACGGCAGCAACAUCCGGAAGAUGAACAGCGAC
GAGACCUUCCUGUACAUCGGCUUCGACAGCCAGGACGGCAAGCGG
GUGAACGAGAUCGAGUUCCUGACCGAGAACCAGAAGAUCUUCGUG
UGCGGCAACAGCAUCAGCGUGGAGGUGCUGGAGGCCAUCAUCGAC
AAGAUCGGCGGCCCCAGCAGCGGCGGCAAGCGGCCCGCCGCCACC
AAGAAGGCCGGCCAGGCCAAGAAGAAGAAGGGCAGCUACCCCUAC
GACGUGCCCGACUACGCCUGAGCGGCCGCUUAAUUAAGCUGCCUU
CUGCGGGGCUUGCCUUCUGGCCAUGCCCUUCUUCUCUCCCUUGCA
CCUGUACCUCUUGGUCUUUGAAUAAAGCCUGAGUAGGAAGUCUAG
AAAAAAAAAAAAAAAAAAAAAAAAAAAAAAAAAAAAAAAAAAAAA
AAAAAAAAAAAAAAAAAAAAAAAAAAAAAAAAAAA

In some embodiments, an expression repressor or site-specific disrupting agent comprises the amino acid sequence of SEQ ID NOs: 203, 208, 73 or 74. In some embodiments, an expression repressor or site-specific disrupting agent described herein comprises an amino acid sequence of SEQ ID NO: 203, 208, 73 or 74, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-MQ1 Protein Sequence (Corresponding to MR-28125):

(SEQ ID NO: 208)
MAPKKKRKVGIHGVPAADKKYSIGLAIGTNSVGWAVITDEYKVP
SKKFKVLGNTDRHSIKKNLIGALLFDSGETAEATRLKRTARRRYT
RRKNRICYLQEIFSNEMAKVDDSFFHRLEESFLVEEDKKHERHPI
FGNIVDEVAYHEKYPTIYHLRKKLVDSTDKADLRLIYLALAHMIK
FRGHFLIEGDLNPDNSDVDKLFIQLVQTYNQLFEENPINASGVDA
KAILSARLSKSRRLENLIAQLPGEKKNGLFGNLIALSLGLTPNFK
SNFDLAEDAKLQLSKDTYDDDLDNLLAQIGDQYADLFLAAKNLSD
AILLSDILRVNTEITKAPLSASMIKRYDEHHQDLTLLKALVRQQL
PEKYKEIFFDQSKNGYAGYIDGGASQEEFYKFIKPILEKMDGTEE
LLVKLNREDLLRKQRTFDNGSIPHQIHLGELHAILRRQEDFYPFL
KDNREKIEKILTFRIPYYVGPLARGNSRFAWMTRKSEETITPWNF
EEVVDKGASAQSFIERMTNFDKNLPNEKVLPKHSLLYEYFTVYNE
LTKVKYVTEGMRKPAFLSGEQKKAIVDLLFKTNRKVTVKQLKEDY
FKKIECFDSVEISGVEDRFNASLGTYHDLLKIIKDKDFLDNEENE
DILEDIVLTLTLFEDREMIEERLKTYAHLFDDKVMKQLKRRRYTG
WGRLSRKLINGIRDKQSGKTILDFLKSDGFANRNFMQLIHDDSLT
FKEDIQKAQVSGQGDSLHEHIANLAGSPAIKKGILQTVKVVDELV
KVMGRHKPENIVIEMARENQTTQKGQKNSRERMKRIEEGIKELGS
QILKEHPVENTQLQNEKLYLYYLQNGRDMYVDQELDINRLSDYDV
AAIVPQSFLKDDSIDNKVLTRSDKARGKSDNVPSEEVVKKMKNYW
RQLLNAKLITQRKFDNLTKAERGGLSELDKAGFIKRQLVETRQIT
KHVAQILDSRMNTKYDENDKLIREVKVITLKSKLVSDFRKDFQFY
KVREINNYHHAHDAYLNAVVGTALIKKYPKLESEFVYGDYKVYDV
RKMIAKSEQEIGKATAKYFFYSNIMNFFKTEITLANGEIRKRPLI
ETNGETGEIVWDKGRDFATVRKVLSMPQVNIVKKTEVQTGGFSKE
SILPKRNSDKLIARKKDWDPKKYGGFDSPTVAYSVLVVAKVEKGK
SKKLKSVKELLGITIMERSSFEKNPIDFLEAKGYKEVKKDLIIKL
PKYSLFELENGRKRMLASAGELQKGNELALPSKYVNFLYLASHYE
KLKGSPEDNEQKQLFVEQHKHYLDEIIEQISEFSKRVILADANLD
KVLSAYNKHRDKPIREQAENIIHLFTLTNLGAPAAFKYFDTTIDR
KRYTSTKEVLDATLIHQSITGLYETRIDLSQLGGDKRPAATKKAG
QAKKKKARDSKVENKTKKLRVFEAFAGIGAQRKALEKVRKDEYEI
VGLAEWYVPAIVMYQAIHNNFHTKLEYKSVSREEMIDYLENKTLS
WNSKNPVSNGYWKRKKDDELKIIYNAIKLSEKEGNIFDIRDLYKR
TLKNIDLLTYSFPCQDLSQQGIQKGMKRGSGTRSGLLWEIERALD
STEKNDLPKYLLMENVGALLHKKNEEELNQWKQKLESLGYQNSIE
VLNAADFGSSQARRRVFMISTLNEFVELPKGDKKPKSIKKVLNKI
VSEKDILNNLLKYNLTEFKKTKSNINKASLIGYSKFNSEGYVYDP
EFTGPTLTASGANSRIKIKDGSNIRKMNSDETFLYIGFDSQDGKR
VNEIEFLTENQKIFVCGNSISVEVLEAIIDKIGGPSSGGKRPAAT
KKAGQAKKKKGSYPYDVPDYA

Sa-dCas9-MQ1 without HA Tag

(SEQ ID NO: 73)
MAPKKKRKVGIHGVPAAAKRNYILGLAIGITSVGYGIIDYETRDV
IDAGVRLFKEANVENNEGRRSKRGARRLKRRRRHRIQRVKKLLFD
YNLLTDHSELSGINPYEARVKGLSQKLSEEEFSAALLHLAKRRGV
HNVNEVEEDTGNELSTKEQISRNSKALEEKYVAELQLERLKKDGE
VRGSINRFKTSDYVKEAKQLLKVQKAYHQLDQSFIDTYIDLLETR
RTYYEGPGEGSPFGWKDIKEWYEMLMGHCTYFPEELRSVKYAYNA
DLYNALNDLNNLVITRDENEKLEYYEKFQIIENVFKQKKKPTLKQ
IAKEILVNEEDIKGYRVTSTGKPEFTNLKVYHDIKDITARKEIIE
NAELLDQIAKILTIYQSSEDIQEELTNLNSELTQEEIEQISNLKG
YTGTHNLSLKAINLILDELWHTNDNQIAIFNRLKLVPKKVDLSQQ
KEIPTTLVDDFILSPVVKRSFIQSIKVINAIIKKYGLPNDIIIEL
AREKNSKDAQKMINEMQKRNRQTNERIEEIIRTTGKENAKYLIEK
IKLHDMQEGKCLYSLEAIPLEDLLNNPFNYEVDAIIPRSVSFDNS
FNNKVLVKQEENSKKGNRTPFQYLSSSDSKISYETFKKHILNLAK
GKGRISKTKKEYLLEERDINRFSVQKDFINRNLVDTRYATRGLMN
LLRSYFRVNNLDVKVKSINGGFTSFLRRKWKFKKERNKGYKHHAE
DALIIANADFIFKEWKKLDKAKKVMENQMFEEKQAESMPEIETEQ
EYKEIFITPHQIKHIKDFKDYKYSHRVDKKPNRKLINDTLYSTRK
DDKGNTLIVNNLNGLYDKDNDKLKKLINKSPEKLLMYHHDPQTYQ
KLKLIMEQYGDEKNPLYKYYEETGNYLTKYSKKDNGPVIKKIKYY
GNKLNAHLDITDDYPNSRNKVVKLSLKPYRFDVYLDNGVYKFVTV
KNLDVIKKENYYEVNSKCYEEAKKLKKISNQAEFIASFYKNDLIK
INGELYRVIGVNNDLLNRIEVNMIDITYREYLENMNDKRPPHIIK
TIASKTQSIKKYSTDILGNLYEVKSKKHPQIIKKGKRPAATKKAG
QAKKKKARDSKVENKTKKLRVFEAFAGIGAQRKALEKVRKDEYEI
VGLAEWYVPAIVMYQAIHNNFHTKLEYKSVSREEMIDYLENKTLS
WNSKNPVSNGYWKRKKDDELKIIYNAIKLSEKEGNIFDIRDLYKR
TLKNIDLLTYSFPCQDLSQQGIQKGMKRGSGTRSGLLWEIERALD
STEKNDLPKYLLMENVGALLHKKNEEELNQWKQKLESLGYQNSIE
VLNAADFGSSQARRRVFMISTLNEFVELPKGDKKPKSIKKVLNKI
VSEKDILNNLLKYNLTEFKKTKSNINKASLIGYSKFNSEGYVYDP
EFTGPTLTASGANSRIKIKDGSNIRKMNSDETFLYIGFDSQDGKR
VNEIEFLTENQKIFVCGNSISVEVLEAIIDKIGGPSSGGKRPAAT
KKAGQAKKKKGS

dCas9-MQ1 without HA Tag

(SEQ ID NO: 74)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDKRPAATKKAGQ
AKKKKARDSKVENKTKKLRVFEAFAGIGAQRKALEKVRKDEYEIV
GLAEWYVPAIVMYQAIHNNFHTKLEYKSVSREEMIDYLENKTLSW
NSKNPVSNGYWKRKKDDELKIIYNAIKLSEKEGNIFDIRDLYKRT
LKNIDLLTYSFPCQDLSQQGIQKGMKRGSGTRSGLLWEIERALDS
TEKNDLPKYLLMENVGALLHKKNEEELNQWKQKLESLGYQNSIEV
LNAADFGSSQARRRVFMISTLNEFVELPKGDKKPKSIKKVLNKIV
SEKDILNNLLKYNLTEFKKTKSNINKASLIGYSKFNSEGYVYDPE
FTGPTLTASGANSRIKIKDGSNIRKMNSDETFLYIGFDSQDGKRV
NEIEFLTENQKIFVCGNSISVEVLEAIIDKIGGPSSGGKRPAATK
KAGQAKKKKGS

In some embodiments, an expression repressor comprises a targeting moiety (e.g., a zinc finger domain, e.g., a zinc finger domain of Table 10), and an effector moiety comprising KRAB, e.g., a KRAB domain. In some embodiments, an expression repressor is encoded by a nucleic acid sequence of Table 16 (e.g., a nucleic acid sequence of any one of SEQ ID NOs: 142-151 or 248-253). In some embodiments, an expression repressor is encoded by a nucleic acid sequence of Table 16, e.g., a nucleic acid sequence of any one of SEQ ID NOs: 142-151 or 248-253, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

It is understood that, when provided as an mRNA, a sequence described herein may comprise an RNA sequence in which each position indicated as a T in Table 16 is occupied by a U. In some embodiments, the 3′ poly-A sequence shown in a sequence of Table 16 is omitted. In some embodiment, a 3′ poly-A sequence is included in the nucleic acid, wherein the 3′ poly-A sequence is up to the length shown in a sequence of Table 16.

TABLE 16
Exemplary expression repressor encoding mRNA

| SEQ ID | | |
|---|---|---|
| NO | Description | Sequence |
| 142 | ZF34-KRAB | AGGAAATAAGAGAGAAAAGAAGAGTAAGAAGAAATATAAGAGCCACC |
| | mRNA (MR- | ATGGCCCCCAAGAAGAAGCGGAAGGTGGGCATCCACGGCGTGCCCGC |
| | 32103) | CGCCGGCAGCAGCGGATCCCTGGAGCCTGGGGAGAAACCCTACAAAT |
| | | GCCCTGAGTGCGGGAAGAGTTTCAGCCACAAGAACGCGTTGCAGAAC |
| | | CACCAAAGGACCCATACTGGAGAGAAACCGTACAAGTGCCCTGAATG |
| | | TGGAAAGTCGTTCTCCCAACGCGCCAACCTGAGAGCTCACCAACGCA |
| | | CTCATACCGGGGAAAAGCCCTATAAGTGCCCGGAATGCGGCAAAAGC |
| | | TTCTCCCGGTCCGACAAGCTTGTGCGGCACCAGCGGACTCACACTGG |
| | | GGAGAAGCCCTACAAGTGCCCAGAATGCGGAAAGAGCTTCAGCCAGG |
| | | CCGGTCATCTGGCCTCGCATCAGCGGACCCACACTGGCGAAAAGCCT |
| | | TACAAGTGTCCGGAGTGCGGAAAGTCCTTCTCCCGGAACGACGCACT |
| | | CACCGAACACCAGCGCACCCACACCGGAGAGAAGCCGTATAAGTGTC |
| | | CAGAATGTGGCAAATCCTTCTCCACATCTGGCAATCTGACCGAGCAC |
| | | CAGAGAACCCATACCGGAGAAAAGCCGTACAAATGTCCCGAATGCGG |
| | | GAAGTCATTTTCCCGCTCGGATCACCTGACCAACCACCAGAGGACTC |
| | | ACACGGGAAAGAAAACCTCAGCTAGCGGCAGCGGCGGCGGCAGCGGC |
| | | GGCGACGCCAAGAGCCTGACCGCCTGGAGCCGGACCCTGGTGACCTT |
| | | CAAGGACGTGTTCGTGGACTTCACCCGGGAGGAGTGGAAGCTGCTGG |
| | | ACACCGCCCAGCAGATCCTGTACCGGAACGTGATGCTGGAGAACTAC |
| | | AAGAACCTGGTGAGCCTGGGCTACCAGCTGACCAAGCCCGACGTGAT |
| | | CCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAG |
| | | AGCAGCGTGAGCGGCGGCAAGCGGCCCGCCGCCACCAAGAAGGCCGG |
| | | CCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCT |
| | | TCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAA |
| 143 | ZF35-KRAB | AGGAAATAAGAGAGAAAAGAAGAGTAAGAAGAAATATAAGAGCCACC |
| | mRNA (MR- | ATGGCCCCCAAGAAGAAGCGGAAGGTGGGCATCCACGGCGTGCCCGC |
| | 32104) | CGCCGGCAGCAGCGGATCCCTTGAGCCCGGCGAAAAGCCCTACAAGT |
| | | GCCCGGAATGCGGAAAATCGTTCAGCTCAAAGAAGGCCTTGACTGAA |
| | | CACCAACGGACCCACACGGGAGAAAAACCGTATAAGTGTCCAGAGTG |
| | | CGGGAAGTCATTCTCCACTTCCCACTCCCTGACCGAGCACCAGCGCA |
| | | CTCACACCGGAGAGAAGCCTTACAAGTGTCCTGAGTGCGGAAAGTCT |
| | | TTCAGCCACAAGAACGCGCTGCAGAACCACCAGAGGACACATACCGG |
| | | CGAAAAACCCTACAAATGCCCGGAGTGTGGAAAGTCCTTCTCGCAAT |
| | | CCTCCAATCTGGTCCGGCACCAGAGAACCCATACTGGGGAAAAACCT |
| | | TACAAATGTCCGGAGTGCGGCAAAAGCTTTTCCCGCTCCGACCACCT |
| | | CACCACTCACCAAAGAACCCACACTGGGGAGAAGCCCTATAAGTGCC |
| | | CTGAGTGTGGGAAGTCCTTCAGCACCAAGAACTCGCTCACCGAACAC |
| | | CAGCGGACTCATACCGGAGAAAAGCCGTACAAGTGCCCAGAATGTGG |
| | | AAAGAGCTTCTCGACCACCGGCAACCTGACTGTGCATCAGCGCACCC |
| | | ATACCGGCAAAAAGACCTCAGCTAGCGGCAGCGGCGGCGGCAGCGGC |
| | | GGCGACGCCAAGAGCCTGACCGCCTGGAGCCGGACCCTGGTGACCTT |
| | | CAAGGACGTGTTCGTGGACTTCACCCGGGAGGAGTGGAAGCTGCTGG |
| | | ACACCGCCCAGCAGATCCTGTACCGGAACGTGATGCTGGAGAACTAC |
| | | AAGAACCTGGTGAGCCTGGGCTACCAGCTGACCAAGCCCGACGTGAT |
| | | CCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAG |
| | | AGCAGCGTGAGCGGCGGCAAGCGGCCCGCCGCCACCAAGAAGGCCGG |
| | | CCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCT |
| | | TCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAA |
| 144 | ZF36-KRAB | AGGAAATAAGAGAGAAAAGAAGAGTAAGAAGAAATATAAGAGCCACC |
| | mRNA (MR- | ATGGCCCCCAAGAAGAAGCGGAAGGTGGGCATCCACGGCGTGCCCGC |
| | 32105) | CGCCGGCAGCAGCGGATCCCTGGAGCCCGGAGAAAAACCCTATAAGT |
| | | GTCCGGAGTGCGGAAAGTCGTTCTCCACTTCCGGCAACCTTGTGCGG |
| | | CACCAGCGAACACATACCGGAGAAAAGCCCTACAAGTGCCCTGAGTG |
| | | TGGGAAGTCCTTCTCCCGGGCGGACAACTTGACCGAGCACCAGAGAA |
| | | CCCATACTGGGGAAAAGCCGTATAAGTGCCCCGAATGTGGAAAGTCC |
| | | TTTAGCCAAAGCGGCGATCTCAGGCGGCATCAGCGCACCCACACTGG |
| | | CGAAAAGCCATACAAGTGCCCGGAGTGTGGAAAATCTTTCTCGCACA |
| | | AGAACGCACTGCAGAATCACCAGCGGACCCATACCGGGGAGAAGCCG |
| | | TACAAGTGTCCTGAATGCGGAAAGAGCTTCTCCGACCCTGGCAACCT |
| | | CGTGCGCCACCAACGCACTCACACCGGTGAAAAGCCTTACAAGTGCC |
| | | CAGAGTGCGGGAAGTCATTCAGCCAGCGGGCCAACCTGAGAGCCCAC |
| | | CAAAGAACCCACACCGGAGAGAAACCCTACAAATGCCCGGAATGCGG |
| | | CAAATCGTTCTCCGACTGCCGCGATCTGGCTCGGCACCAGAGGACTC |
| | | ACACGGGCAAAAAGACCTCAGCTAGCGGCAGCGGCGGCGGCAGCGGC |
| | | GGCGACGCCAAGAGCCTGACCGCCTGGAGCCGGACCCTGGTGACCTT |
| | | CAAGGACGTGTTCGTGGACTTCACCCGGGAGGAGTGGAAGCTGCTGG |
| | | ACACCGCCCAGCAGATCCTGTACCGGAACGTGATGCTGGAGAACTAC |
| | | AAGAACCTGGTGAGCCTGGGCTACCAGCTGACCAAGCCCGACGTGAT |
| | | CCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAG |
| | | AGCAGCGTGAGCGGCGGCAAGCGGCCCGCCGCCACCAAGAAGGCCGG |
| | | CCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCT |
| | | TCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAA |
| 145 | ZF37-KRAB | AGGAAATAAGAGAGAAAAGAAGAGTAAGAAGAAATATAAGAGCCACC |
| | mRNA (MR- | ATGGCCCCCAAGAAGAAGCGGAAGGTGGGCATCCACGGCGTGCCCGC |
| | 32106) | CGCCGGCAGCAGCGGATCCCTGGAACCGGGAGAGAAACCCTATAAGT |
| | | GTCCGGAGTGCGGAAAGTCTTTCTCCCGCAACGACGCGCTTACCGAG |
| | | CACCAGCGCACCCACACTGGAGAAAAACCCTACAAATGCCCTGAATG |
| | | TGGCAAATCATTCTCCGATAAGAAGGACTTGACCAGACACCAGCGGA |
| | | CCCACACCGGAGAAAAGCCGTATAAGTGCCCCGAGTGTGGGAAGTCG |
| | | TTCAGCCGGGCCGATAACCTGACGGAACACCAGAGAACTCATACCGG |
| | | GGAGAAGCCGTACAAGTGTCCTGAATGCGGGAAGTCCTTTAGCCACA |
| | | AGAACGCCCTGCAAAACCACCAACGGACCCATACCGGAGAGAAGCCT |
| | | TACAAGTGCCCGGAATGCGGAAAGAGCTTCTCGACCTCCCATTCGCT |
| | | GACTGAGCATCAGAGGACACACACCGGCGAAAAGCCATACAAGTGCC |
| | | CTGAGTGCGGCAAATCCTTCTCCACTTCCGGCAATCTCGTGCGGCAC |
| | | CAGAGGACTCACACCGGGGAAAAGCCCTACAAGTGCCCAGAATGTGG |
| | | AAAGTCCTTCAGCCGCGAGGACAACCTCCACACCCATCAGCGCACTC |
| | | ACACTGGCAAAAAGACCTCAGCTAGCGGCAGCGGCGGCGGCAGCGGC |
| | | GGCGACGCCAAGAGCCTGACCGCCTGGAGCCGGACCCTGGTGACCTT |
| | | CAAGGACGTGTTCGTGGACTTCACCCGGGAGGAGTGGAAGCTGCTGG |
| | | ACACCGCCCAGCAGATCCTGTACCGGAACGTGATGCTGGAGAACTAC |
| | | AAGAACCTGGTGAGCCTGGGCTACCAGCTGACCAAGCCCGACGTGAT |
| | | CCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAG |
| | | AGCAGCGTGAGCGGCGGCAAGCGGCCCGCCGCCACCAAGAAGGCCGG |
| | | CCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCT |
| | | TCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAA |
| 146 | ZF38-KRAB | AGGAAATAAGAGAGAAAAGAAGAGTAAGAAGAAATATAAGAGCCACC |
| | mRNA (MR- | ATGGCCCCCAAGAAGAAGCGGAAGGTGGGCATCCACGGCGTGCCCGC |
| | 32107) | CGCCGGCAGCAGCGGATCCCTGGAGCCTGGCGAAAAGCCTTACAAGT |
| | | GTCCCGAATGCGGCAAATCGTTCTCACGGAAGGACAACCTTAAGAAC |
| | | CACCAGAGGACCCATACCGGAGAAAAGCCATACAAATGCCCGGAGTG |
| | | CGGGAAGTCCTTCTCCCGCGCCGACAACCTGACTGAGCACCAACGGA |
| | | CCCATACAGGGGAGAAGCCCTATAAGTGCCCGGAATGTGGAAAGTCG |
| | | TTCTCGGACCCCGGTGCACTCGTGCGCCATCAGAGGACGCACACCGG |
| | | GGAAAAGCCCTACAAATGTCCGGAATGCGGAAAGTCCTTTTCCGACA |
| | | AGAAGGATTTGACCAGACACCAGCGCACTCACACCGGAGAGAAGCCG |
| | | TACAAGTGCCCAGAGTGCGGCAAAAGCTTCTCCGATCCTGGAGCTCT |
| | | GGTCCGGCATCAGCGGACCCACACCGGTGAAAAGCCGTATAAGTGTC |
| | | CGGAGTGTGGAAAGAGCTTCTCCCGGTCCGACAATCTCGTGCGGCAC |
| | | CAACGCACCCATACTGGCGAAAAACCCTACAAGTGCCCTGAGTGCGG |
| | | AAAAAGCTTCAGCTCTCCTGCCGACCTGACTAGACACCAGAGAACCC |
| | | ACACTGGCAAAAAGACCTCAGCTAGCGGCAGCGGCGGCGGCAGCGGC |
| | | GGCGACGCCAAGAGCCTGACCGCCTGGAGCCGGACCCTGGTGACCTT |
| | | CAAGGACGTGTTCGTGGACTTCACCCGGGAGGAGTGGAAGCTGCTGG |
| | | ACACCGCCCAGCAGATCCTGTACCGGAACGTGATGCTGGAGAACTAC |
| | | AAGAACCTGGTGAGCCTGGGCTACCAGCTGACCAAGCCCGACGTGAT |
| | | CCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAG |
| | | AGCAGCGTGAGCGGCGGCAAGCGGCCCGCCGCCACCAAGAAGGCCGG |
| | | CCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCT |
| | | TCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAA |
| 147 | ZF39-KRAB | AGGAAATAAGAGAGAAAAGAAGAGTAAGAAGAAATATAAGAGCCACC |
| | mRNA (MR- | ATGGCCCCCAAGAAGAAGCGGAAGGTGGGCATCCACGGCGTGCCCGC |
| | 32108) | CGCCGGCAGCAGCGGATCCCTGGAACCCGGAGAGAAGCCGTATAAGT |
| | | GTCCTGAATGCGGAAAGTCCTTCTCCCGCTCGGACGAGCTTGTGCGG |
| | | CACCAGCGCACTCACACTGGCGAAAAGCCGTACAAATGCCCCGAATG |
| | | CGGGAAGTCCTTCAGCCAATCGGGAGATCTGCGCAGACATCAGCGGA |
| | | CCCACACCGGAGAAAAGCCTTATAAGTGCCCTGAGTGCGGGAAATCG |
| | | TTCTCCCAACGGGCGAACCTCAGGGCCCACCAACGGACTCACACGGG |
| | | GGAAAAGCCCTACAAGTGTCCGGAGTGTGGAAAGTCATTCTCCACCC |
| | | ACCTGGACCTGATCAGGCACCAGCGGACACATACCGGAGAGAAACCG |
| | | TACAAGTGCCCGGAATGTGGAAAGAGCTTCTCAAGATCCGACCACTT |
| | | GACCACCCATCAGCGCACCCATACTGGGGAGAAGCCTTACAAATGTC |
| | | CCGAATGTGGCAAATCGTTTTCCACCTCCGGCAACCTGACCGAGCAT |
| | | CAGAGGACTCACACCGGCGAAAAACCATACAAGTGCCCAGAGTGCGG |
| | | CAAAAGCTTCAGCCAGTCCGGCGATCTCCGGAGACACCAGCGAACCC |
| | | ACACTGGAAAGAAAACCTCTGCTAGCGGCAGCGGCGGCGGCAGCGGC |
| | | GGCGACGCCAAGAGCCTGACCGCCTGGAGCCGGACCCTGGTGACCTT |
| | | CAAGGACGTGTTCGTGGACTTCACCCGGGAGGAGTGGAAGCTGCTGG |
| | | ACACCGCCCAGCAGATCCTGTACCGGAACGTGATGCTGGAGAACTAC |
| | | AAGAACCTGGTGAGCCTGGGCTACCAGCTGACCAAGCCCGACGTGAT |
| | | CCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAG |
| | | AGCAGCGTGAGCGGCGGCAAGCGGCCCGCCGCCACCAAGAAGGCCGG |
| | | CCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCT |
| | | TCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAA |
| 148 | ZF40-KRAB | AGGAAATAAGAGAGAAAAGAAGAGTAAGAAGAAATATAAGAGCCACC |
| | mRNA (MR- | ATGGCCCCCAAGAAGAAGCGGAAGGTGGGCATCCACGGCGTGCCCGC |
| | 32109) | CGCCGGCAGCAGCGGATCCCTGGAGCCTGGCGAAAAACCGTACAAGT |
| | | GCCCCGAATGTGGAAAGTCTTTCTCCCGCAACGACGCGTTGACCGAA |
| | | CACCAGCGGACCCATACCGGTGAAAAGCCTTACAAGTGCCCTGAGTG |
| | | CGGCAAAAGCTTCTCCCGCCGCGATGAACTGAACGTGCACCAACGCA |
| | | CCCACACTGGAGAAAAGCCATACAAATGCCCGGAGTGTGGGAAGTCC |
| | | TTCTCCCGGTCCGATGAGCTTGTGCGGCACCAGAGAACTCACACAGG |
| | | GGAAAAGCCCTATAAGTGCCCAGAGTGCGGGAAGTCATTCAGCCGGG |
| | | CCGACAACCTGACCGAGCACCAAAGGACTCACACCGGAGAGAAGCCG |
| | | TATAAGTGTCCTGAGTGTGGAAAGAGCTTCAGCACCTCCCACTCCCT |
| | | CACCGAACATCAGCGGACTCATACTGGGGAGAAGCCCTACAAGTGTC |
| | | CCGAATGCGGCAAATCCTTTTCGGAACGGTCGCACCTGAGAGAGCAT |
| | | CAGAGGACGCACACCGGCGAAAAGCCGTACAAATGTCCGGAGTGCGG |
| | | AAAGTCGTTCTCCACCCACCTCGACCTGATCAGACACCAGCGCACCC |
| | | ATACTGGCAAAAAGACCTCAGCTAGCGGCAGCGGCGGCGGCAGCGGC |
| | | GGCGACGCCAAGAGCCTGACCGCCTGGAGCCGGACCCTGGTGACCTT |
| | | CAAGGACGTGTTCGTGGACTTCACCCGGGAGGAGTGGAAGCTGCTGG |
| | | ACACCGCCCAGCAGATCCTGTACCGGAACGTGATGCTGGAGAACTAC |
| | | AAGAACCTGGTGAGCCTGGGCTACCAGCTGACCAAGCCCGACGTGAT |
| | | CCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAG |
| | | AGCAGCGTGAGCGGCGGCAAGCGGCCCGCCGCCACCAAGAAGGCCGG |
| | | CCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCT |
| | | TCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAA |
| 149 | ZF41-KRAB | AGGAAATAAGAGAGAAAAGAAGAGTAAGAAGAAATATAAGAGCCACC |
| | mRNA (MR- | ATGGCCCCCAAGAAGAAGCGGAAGGTGGGCATCCACGGCGTGCCCGC |
| | 32110) | CGCCGGCAGCAGCGGATCCCTGGAGCCCGGAGAGAAACCGTACAAGT |
| | | GTCCAGAGTGCGGCAAATCCTTCTCGACCTCCGGCAACCTGACTGAA |
| | | CACCAGCGCACTCACACTGGAGAAAAGCCGTACAAATGTCCTGAATG |
| | | CGGAAAGAGCTTCAGCCAGTCCTCCTCACTTGTGCGCCACCAGAGGA |
| | | CCCACACCGGAGAAAAACCGTATAAGTGTCCTGAGTGTGGGAAGTCA |
| | | TTCTCAAGAAGCGATCACCTCACCACCCATCAGCGGACCCATACGGG |
| | | GGAGAAGCCCTACAAATGCCCGGAGTGCGGAAAGTCGTTCAGCCGGT |
| | | CCGACCACCTGACCACCCACCAAAGAACTCACACCGGCGAAAAGCCT |
| | | TACAAGTGCCCTGAATGTGGAAAGTCCTTTTCCCAATCGTCCTCCTT |
| | | GGTCCGGCATCAGAGGACTCACACAGGAGAGAAGCCATACAAGTGCC |
| | | CCGAATGCGGGAAGTCCTTCAGCTCGAAGAAGGCCCTGACTGAGCAC |
| | | CAGCGGACTCATACCGGGGAAAAGCCCTATAAGTGCCCGGAATGCGG |
| | | CAAAAGCTTCTCGACCAAGAACTCCCTCACCGAGCATCAGCGCACCC |
| | | ACACTGGCAAAAAGACCTCTGCTAGCGGCAGCGGCGGCGGCAGCGGC |
| | | GGCGACGCCAAGAGCCTGACCGCCTGGAGCCGGACCCTGGTGACCTT |
| | | CAAGGACGTGTTCGTGGACTTCACCCGGGAGGAGTGGAAGCTGCTGG |
| | | ACACCGCCCAGCAGATCCTGTACCGGAACGTGATGCTGGAGAACTAC |
| | | AAGAACCTGGTGAGCCTGGGCTACCAGCTGACCAAGCCCGACGTGAT |
| | | CCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAG |
| | | AGCAGCGTGAGCGGCGGCAAGCGGCCCGCCGCCACCAAGAAGGCCGG |
| | | CCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCT |
| | | TCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAA |
| 150 | ZF42-KRAB | AGGAAATAAGAGAGAAAAGAAGAGTAAGAAGAAATATAAGAGCCACC |
| | mRNA (MR- | ATGGCCCCCAAGAAGAAGCGGAAGGTGGGCATCCACGGCGTGCCCGC |
| | 32111) | CGCCGGCAGCAGCGGATCCCTGGAACCCGGAGAGAAGCCCTATAAGT |
| | | GTCCGGAGTGCGGCAAATCCTTTTCCACCCATCTCGACCTGATTAGG |
| | | CACCAGAGAACTCACACTGGGGAGAAGCCATACAAGTGCCCCGAATG |
| | | TGGAAAGTCTTTCTCCGATCCGGGCAACCTTGTCCGGCACCAGAGGA |
| | | CCCACACGGGCGAAAAACCTTATAAGTGCCCTGAGTGTGGGAAGTCC |
| | | TTCAGCCGGAAGGACAACCTGAAGAACCACCAAAGAACACACACCGG |
| | | AGAAAAGCCCTACAAATGCCCAGAGTGCGGGAAGTCGTTCTCCGACA |
| | | AGAAGGATTTGACCCGGCACCAACGGACCCACACTGGCGAAAAGCCG |
| | | TACAAATGTCCCGAATGCGGAAAGAGCTTCTCGCGCTCCGACGAGCT |
| | | CGTGCGCCATCAGCGGACTCATACCGGAGAGAAACCGTACAAGTGTC |
| | | CTGAGTGCGGAAAGTCCTTCTCAACCCACCTGGACCTGATCAGACAC |
| | | CAGCGCACTCACACCGGGGAAAAGCCTTACAAGTGCCCGGAATGCGG |
| | | CAAAAGCTTCTCCCAATCGAGCTCCCTCGTGCGGCATCAGCGCACCC |
| | | ATACCGGAAAGAAAACCTCAGCTAGCGGCAGCGGCGGCGGCAGCGGC |
| | | GGCGACGCCAAGAGCCTGACCGCCTGGAGCCGGACCCTGGTGACCTT |
| | | CAAGGACGTGTTCGTGGACTTCACCCGGGAGGAGTGGAAGCTGCTGG |
| | | ACACCGCCCAGCAGATCCTGTACCGGAACGTGATGCTGGAGAACTAC |
| | | AAGAACCTGGTGAGCCTGGGCTACCAGCTGACCAAGCCCGACGTGAT |
| | | CCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAG |
| | | AGCAGCGTGAGCGGCGGCAAGCGGCCCGCCGCCACCAAGAAGGCCGG |
| | | CCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCT |
| | | TCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAA |
| 151 | ZF43-KRAB | AGGAAATAAGAGAGAAAAGAAGAGTAAGAAGAAATATAAGAGCCACC |
| | mRNA (MR- | ATGGCCCCCAAGAAGAAGCGGAAGGTGGGCATCCACGGCGTGCCCGC |
| | 32112) | CGCCGGCAGCAGCGGATCCTTGGAACCGGGGGAAAAGCCGTACAAGT |
| | | GCCCCGAATGTGGCAAATCATTCTCTACCTCCCACTCACTGACCGAG |
| | | CACCAGAGGACCCACACCGGAGAGAAACCCTACAAGTGTCCAGAATG |
| | | CGGAAAGTCCTTCTCGCAAGCCGGTCACCTTGCGTCCCACCAACGCA |
| | | CTCATACTGGAGAAAAACCATACAAGTGCCCGGAATGCGGCAAAAGC |
| | | TTCAGCACCAAGAACAGCCTGACTGAGCATCAGAGGACTCACACCGG |
| | | CGAAAAGCCCTATAAGTGTCCTGAGTGCGGCAAATCCTTCTCCGATT |
| | | GTCGGGACCTGGCCAGACACCAGCGCACTCACACAGGAGAAAAGCCT |
| | | TATAAGTGCCCTGAGTGTGGAAAGTCGTTTAGCCAAGCTGGTCACCT |
| | | GGCATCCCATCAGCGGACTCATACCGGGGAGAAGCCTTACAAATGTC |
| | | CGGAGTGCGGGAAGTCGTTCAGCCAGTCCTCCTCCCTCGTGCGGCAC |
| | | CAGAGAACCCACACTGGAGAGAAGCCCTACAAATGCCCTGAATGTGG |
| | | AAAGAGCTTCTCCCGCTCGGACCACCTCACCACGCACCAGCGGACCC |
| | | ATACCGGCAAAAAGACCTCAGCTAGCGGCAGCGGCGGCGGCAGCGGC |
| | | GGCGACGCCAAGAGCCTGACCGCCTGGAGCCGGACCCTGGTGACCTT |
| | | CAAGGACGTGTTCGTGGACTTCACCCGGGAGGAGTGGAAGCTGCTGG |
| | | ACACCGCCCAGCAGATCCTGTACCGGAACGTGATGCTGGAGAACTAC |
| | | AAGAACCTGGTGAGCCTGGGCTACCAGCTGACCAAGCCCGACGTGAT |
| | | CCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAG |
| | | AGCAGCGTGAGCGGCGGCAAGCGGCCCGCCGCCACCAAGAAGGCCGG |
| | | CCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCT |
| | | TCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAA |
| 248 | ZF44-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32694) | GGCTCGCTGGAGCCCGGGGAGAAGCCATATAAGTGTCCCGAAT |
| | | GCGGAAAGTCCTTCAGCGACTCCGGCAACCTTAGGGTCCATCA |
| | | GCGAACCCACACGGGGGAGAAACCGTACAAATGCCCGGAGTGT |
| | | GGGAAGTCCTTTTCCCAACTGGCTCACTTGCGCGCGCACCAGA |
| | | GAACTCATACTGGAGAAAAGCCCTACAAGTGTCCGGAATGTGG |
| | | AAAGAGCTTCAGCACCAAGAACTCCCTCACCGAGCACCAGCGG |
| | | ACCCATACCGGAGAAAAACCTTACAAGTGCCCTGAATGCGGAA |
| | | AGTCCTTCAGCACGTCCGGTTCCCTGGTTCGGCACCAGAGGAC |
| | | CCACACTGGAGAGAAGCCTTACAAATGTCCAGAGTGCGGCAAA |
| | | AGCTTCTCGAGCCCCGCCGATTTGACCCGCCACCAAAGAACTC |
| | | ACACCGGGGAAAAGCCGTACAAGTGCCCGGAATGCGGCAAATC |
| | | CTTCTCACAATCCTCCTCTCTTGTCCGGCACCAGCGCACACAT |
| | | ACTGGCGAAAAACCATATAAGTGCCCAGAATGCGGGAAGTCGT |
| | | TTAGCCAGTCGGGCGATCTGAGGAGACATCAGCGCACCCACAC |
| | | CGGAAAGAAAACCAGCGCTTCCGGTTCGGGTGGAGGATCGGGC |
| | | GGAGATGCCAAGTCACTGACCGCATGGTCCCGGACCCTTGTGA |
| | | CCTTCAAGGACGTGTTCGTGGACTTCACCAGAGAGGAGTGGAA |
| | | GCTGCTGGACACCGCGCAACAGATTCTCTACCGGAACGTGATG |
| | | CTGGAGAACTACAAGAATCTGGTGTCCCTGGGCTACCAGCTGA |
| | | CCAAGCCGGACGTGATCCTCCGCCTCGAAAAGGGGGAGGAGCC |
| | | TTGGCTCGTGGAGCGGGAAATCCACCAGGAGACTCACCCGGAC |
| | | TCCGAGACTGCCTTCGAAATCAAGTCATCGGTGTCCGGTGGAA |
| | | AGCGGCCTGCAGCGACTAAGAAGGCCGGACAGGCCAAGAAGAA |
| | | GAAGGGCTCATACCCGTACGATGTGCCCGACTACGCCTAATGA |
| | | GCGGCCGCTTAATTAAGCTGCCTTCTGCGGGGCTTGCCTTCTG |
| | | GCCATGCCCTTCTTCTCTCCCTTGCACCTGTACCTCTTGGTCT |
| | | TTGAATAAAGCCTGAGTAGGAAGTCTAGAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAA |
| 249 | ZF45-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32695) | GGCTCGCTGGAGCCCGGCGAAAAGCCCTACAAGTGTCCGGAGT |
| | | GCGGAAAGAGCTTCTCCCGCTCCGATAACCTTGTGCGCCATCA |
| | | GCGAACCCACACGGGGGAGAAACCGTACAAATGCCCGGAGTGT |
| | | GGGAAGTCCTTTTCCCAAAACAGCACCCTCACCGAGCACCAGC |
| | | GGACCCATACCGGAGAAAAACCTTACAAGTGCCCTGAATGCGG |
| | | AAAGTCCTTCAGCCAATCCAGCTCCCTGGTTCGGCACCAGAGG |
| | | ACCCACACTGGAGAGAAGCCTTACAAATGTCCAGAGTGCGGCA |
| | | AAAGCTTCTCGCAAAAGTCGTCGTTGATTGCCCACCAAAGAAC |
| | | TCACACCGGGGAAAAGCCGTACAAGTGCCCGGAATGCGGCAAA |
| | | TCCTTCTCAACTTCCGGCAACCTTGTCCGGCACCAGCGCACAC |
| | | ATACTGGCGAAAAACCATATAAGTGCCCAGAATGCGGGAAGTC |
| | | GTTTAGCTCCCCCGCTGATCTGACCAGACATCAGCGGACACAC |
| | | ACCGGAGAGAAACCCTATAAGTGTCCCGAATGTGGAAAATCCT |
| | | TCAGCCGCTCGGACGAACTGGTCCGCCATCAGCGGACACACAC |
| | | CGGAAAGAAAACTTCAGCCTCTGGCAGCGGAGGAGGTTCCGGT |
| | | GGAGATGCCAAGTCACTGACCGCATGGTCCCGGACCCTTGTGA |
| | | CCTTCAAGGACGTGTTCGTGGACTTCACCAGAGAGGAGTGGAA |
| | | GCTGCTGGACACCGCGCAACAGATTCTCTACCGGAACGTGATG |
| | | CTGGAGAACTACAAGAATCTGGTGTCCCTGGGCTACCAGCTGA |
| | | CCAAGCCGGACGTGATCCTCCGCCTCGAAAAGGGGGAGGAGCC |
| | | TTGGCTCGTGGAGCGGGAAATCCACCAGGAGACTCACCCGGAC |
| | | TCCGAGACTGCCTTCGAAATCAAGTCATCGGTGTCCGGTGGAA |
| | | AGCGGCCTGCAGCGACTAAGAAGGCCGGACAGGCCAAGAAGAA |
| | | GAAGGGCTCATACCCCTACGACGTGCCGGACTACGCCTAATGA |
| | | GCGGCCGCTTAATTAAGCTGCCTTCTGCGGGGCTTGCCTTCTG |
| | | GCCATGCCCTTCTTCTCTCCCTTGCACCTGTACCTCTTGGTCT |
| | | TTGAATAAAGCCTGAGTAGGAAGTCTAGAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAA |
| 250 | ZF46-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32696) | GGCTCGCTGGAACCGGGAGAGAAGCCATACAAATGCCCCGAGT |
| | | GTGGAAAGTCCTTTTCGACTTCCGGTTCGCTTGTGCGCCATCA |
| | | GCGAACCCACACGGGGGAGAAACCGTACAAATGCCCGGAGTGT |
| | | GGGAAGTCCTTTTCCCAACGGGCCAACCTGAGAGCCCACCAAA |
| | | GGACGCATACCGGCGAAAAGCCCTACAAGTGTCCCGAATGTGG |
| | | AAAGAGCTTCTCTCGCACTGACACCCTCCGGGATCACCAGCGG |
| | | ACCCATACCGGAGAAAAACCTTACAAGTGCCCTGAATGCGGAA |
| | | AGTCCTTCAGCCGGAGGGATGAACTGAACGTCCACCAGAGGAC |
| | | CCACACTGGAGAGAAGCCTTACAAATGTCCAGAGTGCGGCAAA |
| | | AGCTTCTCGCATCGCACCACCTTGACCAACCACCAAAGAACTC |
| | | ACACCGGGGAAAAGCCGTACAAGTGCCCGGAATGCGGCAAATC |
| | | CTTCTCACGCTCCGACAAGCTTACTGAACACCAGCGCACACAT |
| | | ACTGGCGAAAAACCATATAAGTGCCCAGAATGCGGGAAGTCGT |
| | | TTAGCCAGAAGTCCAGCCTGATTGCTCACCAGCGCACCCACAC |
| | | CGGAAAGAAAACCTCCGCATCCGGAAGCGGTGGAGGAAGTGGC |
| | | GGGGATGCCAAGTCACTGACCGCATGGTCCCGGACCCTTGTGA |
| | | CCTTCAAGGACGTGTTCGTGGACTTCACCAGAGAGGAGTGGAA |
| | | GCTGCTGGACACCGCGCAACAGATTCTCTACCGGAACGTGATG |
| | | CTGGAGAACTACAAGAATCTGGTGTCCCTGGGCTACCAGCTGA |
| | | CCAAGCCGGACGTGATCCTCCGCCTCGAAAAGGGGGAGGAGCC |
| | | TTGGCTCGTGGAGCGGGAAATCCACCAGGAGACTCACCCGGAC |
| | | TCCGAGACTGCCTTCGAAATCAAGTCATCGGTGTCCGGTGGAA |
| | | AGCGGCCTGCAGCGACTAAGAAGGCCGGACAGGCCAAGAAGAA |
| | | GAAGGGCTCATACCCGTACGATGTGCCCGACTATGCCTAATGA |
| | | GCGGCCGCTTAATTAAGCTGCCTTCTGCGGGGCTTGCCTTCTG |
| | | GCCATGCCCTTCTTCTCTCCCTTGCACCTGTACCTCTTGGTCT |
| | | TTGAATAAAGCCTGAGTAGGAAGTCTAGAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAA |
| 251 | ZF47-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32697) | GGCTCGCTGGAACCTGGGGAGAAGCCATATAAGTGCCCTGAGT |
| | | GTGGAAAGTCCTTCTCCCGGGCTGACAATCTGACCGAACACCA |
| | | GCGGACTCACACTGGAGAGAAGCCGTACAAATGCCCCGAGTGC |
| | | GGAAAGTCGTTCTCCCGCTCCGACCATCTCACCACCCATCAGA |
| | | GAACCCACACCGGGGAAAAGCCATACAAGTGTCCTGAATGCGG |
| | | CAAATCATTCAGCCGCTCGGACAACCTCGTGCGGCATCAACGA |
| | | ACCCACACGGGAGAGAAACCGTATAAATGCCCGGAGTGCGGCA |
| | | AAAGCTTTAGCCAGAACTCCACTCTGACGGAGCACCAACGCAC |
| | | CCATACCGGAGAAAAGCCTTACAAGTGCCCAGAGTGCGGTAAA |
| | | AGCTTCAGCCAGTCCTCCTCACTCGTGCGCCACCAGAGAACTC |
| | | ATACTGGCGAAAAGCCGTATAAGTGCCCGGAATGTGGAAAGAG |
| | | CTTCAGTCAGAAGTCCAGCCTGATTGCGCACCAGAGGACTCAT |
| | | ACCGGCGAAAAACCCTACAAATGTCCGGAATGCGGAAAATCGT |
| | | TCTCAACTTCCGGCAACCTTGTGCGGCACCAGCGCACACACAC |
| | | CGGCAAAAAGACTTCGGCATCCGGTTCGGGAGGAGGATCAGGA |
| | | GGAGATGCCAAGTCACTGACCGCATGGTCCCGGACCCTTGTGA |
| | | CCTTCAAGGACGTGTTCGTGGACTTCACCAGAGAGGAGTGGAA |
| | | GCTGCTGGACACCGCGCAACAGATTCTCTACCGGAACGTGATG |
| | | CTGGAGAACTACAAGAATCTGGTGTCCCTGGGCTACCAGCTGA |
| | | CCAAGCCGGACGTGATCCTCCGCCTCGAAAAGGGGGAGGAGCC |
| | | TTGGCTCGTGGAGCGGGAAATCCACCAGGAGACTCACCCGGAC |
| | | TCCGAGACTGCCTTCGAAATCAAGTCATCGGTGTCCGGTGGAA |
| | | AGCGGCCTGCAGCGACTAAGAAGGCCGGACAGGCCAAGAAGAA |
| | | GAAGGGCTCATACCCTTACGATGTGCCGGATTACGCCTAATGA |
| | | GCGGCCGCTTAATTAAGCTGCCTTCTGCGGGGCTTGCCTTCTG |
| | | GCCATGCCCTTCTTCTCTCCCTTGCACCTGTACCTCTTGGTCT |
| | | TTGAATAAAGCCTGAGTAGGAAGTCTAGAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAA |
| 252 | ZF48-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32698) | GGCTCGCTCGAACCCGGAGAAAAGCCCTACAAATGCCCAGAGT |
| | | GCGGAAAGAGCTTTTCCCGCTCCGATCATCTCACCACGCACCA |
| | | GCGGACCCATACCGGAGAAAAACCTTACAAGTGCCCTGAATGC |
| | | GGAAAGTCCTTCAGCGACCCCGGTCACCTGGTTCGGCACCAGA |
| | | GGACCCACACTGGAGAGAAGCCTTACAAATGTCCAGAGTGCGG |
| | | CAAAAGCTTCTCGCACAAGAACGCCTTGCAAAACCACCAACGC |
| | | ACTCATACCGGCGAAAAGCCATACAAGTGTCCGGAGTGTGGGA |
| | | AGTCCTTCTCGCGACGCGACGAACTGAACGTCCACCAAAGAAC |
| | | TCACACCGGGGAAAAGCCGTACAAGTGCCCGGAATGCGGCAAA |
| | | TCCTTCTCACAACGCGCCAACCTTAGGGCTCACCAGCGCACAC |
| | | ATACTGGCGAAAAACCATATAAGTGCCCAGAATGCGGGAAGTC |
| | | GTTTAGCACGACTGGCAACCTGACCGTCCATCAGCGGACACAC |
| | | ACCGGAGAGAAACCCTATAAGTGTCCCGAATGTGGAAAATCCT |
| | | TCAGCTCTAAGAAGCATCTGGCCGAGCACCAGAGAACCCACAC |
| | | TGGGAAGAAAACCAGCGCATCAGGAAGCGGAGGAGGTTCCGGT |
| | | GGCGATGCCAAGTCACTGACCGCATGGTCCCGGACCCTTGTGA |
| | | CCTTCAAGGACGTGTTCGTGGACTTCACCAGAGAGGAGTGGAA |
| | | GCTGCTGGACACCGCGCAACAGATTCTCTACCGGAACGTGATG |
| | | CTGGAGAACTACAAGAATCTGGTGTCCCTGGGCTACCAGCTGA |
| | | CCAAGCCGGACGTGATCCTCCGCCTCGAAAAGGGGGAGGAGCC |
| | | TTGGCTCGTGGAGCGGGAAATCCACCAGGAGACTCACCCGGAC |
| | | TCCGAGACTGCCTTCGAAATCAAGTCATCGGTGTCCGGTGGAA |
| | | AGCGGCCTGCAGCGACTAAGAAGGCCGGACAGGCCAAGAAGAA |
| | | GAAGGGCTCATACCCTTACGACGTCCCGGATTACGCGTAATGA |
| | | GCGGCCGCTTAATTAAGCTGCCTTCTGCGGGGCTTGCCTTCTG |
| | | GCCATGCCCTTCTTCTCTCCCTTGCACCTGTACCTCTTGGTCT |
| | | TTGAATAAAGCCTGAGTAGGAAGTCTAGAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAA |
| 253 | ZF49-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32699) | GGCTCGCTGGAGCCTGGAGAGAAGCCGTATAAGTGCCCAGAAT |
| | | GTGGAAAGTCATTCAGCTCCAAGAAGGCTCTGACCGAACACCA |
| | | ACGGACGCACACCGGAGAAAAACCCTACAAATGCCCTGAATGC |
| | | GGGAAGTCCTTTTCCACCTCCGGCAACCTGACCGAGCACCAGC |
| | | GGACTCACACTGGAGAGAAACCTTACAAGTGCCCTGAGTGTGG |
| | | AAAGAGCTTCTCCCATCGCACTACCCTTACCAATCACCAACGC |
| | | ACCCATACCGGCGAAAAACCATACAAATGTCCAGAGTGCGGCA |
| | | AATCCTTCAGCCGCAACGATGCGCTCACCGAGCATCAGCGCAC |
| | | TCATACTGGCGAAAAGCCCTATAAGTGTCCGGAATGCGGAAAG |
| | | TCGTTCAGCGACTCCGGAAACCTTAGAGTGCACCAGAGGACAC |
| | | ACACTGGGGAAAAGCCGTACAAGTGTCCCGAATGCGGCAAAAG |
| | | CTTCAGCCAGTCCGGGGATCTGCGGAGGCACCAGCGAACCCAT |
| | | ACGGGAGAAAAGCCTTATAAATGCCCGGAGTGCGGAAAGAGTT |
| | | TCTCGCGGAGCGACCACCTGACCAACCACCAGAGAACCCACAC |
| | | TGGCAAAAAGACCTCGGCATCCGGTTCAGGAGGAGGGTCCGGA |
| | | GGAGATGCCAAGTCACTGACCGCATGGTCCCGGACCCTTGTGA |
| | | CCTTCAAGGACGTGTTCGTGGACTTCACCAGAGAGGAGTGGAA |
| | | GCTGCTGGACACCGCGCAACAGATTCTCTACCGGAACGTGATG |
| | | CTGGAGAACTACAAGAATCTGGTGTCCCTGGGCTACCAGCTGA |
| | | CCAAGCCGGACGTGATCCTCCGCCTCGAAAAGGGGGAGGAGCC |
| | | TTGGCTCGTGGAGCGGGAAATCCACCAGGAGACTCACCCGGAC |
| | | TCCGAGACTGCCTTCGAAATCAAGTCATCGGTGTCCGGTGGAA |
| | | AGCGGCCTGCAGCGACTAAGAAGGCCGGACAGGCCAAGAAGAA |
| | | GAAGGGCTCATACCCGTACGACGTGCCCGATTACGCTTAATGA |
| | | GCGGCCGCTTAATTAAGCTGCCTTCTGCGGGGCTTGCCTTCTG |
| | | GCCATGCCCTTCTTCTCTCCCTTGCACCTGTACCTCTTGGTCT |
| | | TTGAATAAAGCCTGAGTAGGAAGTCTAGAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAA |

In some embodiments, an expression repressor is encoded by a nucleic acid sequence of SEQ ID NO: 142, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 143, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 144, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 145, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 146, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 147, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 148, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 149, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 150, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 151, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

In some embodiments, an expression repressor comprises an amino acid sequence of SEQ ID NO: 248 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 249 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 250 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 251 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 252 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 253 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto.

In some embodiments, an expression repressor comprises a targeting moiety (e.g., a zinc finger domain, e.g., a zinc finger domain of Table 10), and an effector moiety comprising KRAB, e.g., a KRAB domain. In some embodiments, an expression repressor comprises an amino acid sequence of Table 17 (e.g., amino acid sequence of any one of SEQ ID NOs: 152-161 or 164-169. In some embodiments, an expression repressor comprises an amino acid sequence of Table 17, e.g., an amino acid sequence of any one of SEQ ID NOs: 152-161 or 164-169, or an amino acid sequence with at least 80%, 85%, 90%, 95%, 99%, or 100% identity thereto, or an amino acid sequence having no more than_20, 19, 18, 17, 16, 15, 14, 13, 12, 11, 10, 9, 8, 7, 6, 5, 4, 3, 2, or 1 positions of difference thereto.

TABLE 17
Exemplary expression repressor polypeptide sequences
(bold italics: targeting moiety; underline: effector moiety)

| SEQ ID | | |
|---|---|---|
| NO | Description | Sequence |
| 152 | ZF34-KRAB | MAPKKKRKVGIHGVPAAGSSGSLEPGEKPYKCPECGKSFSHKNAL |
| | (MR-32103) | QNHQRTHTGEKPYKCPECGKSFSQRANLRAHQRTHTGEKPYKCPE |
| | | CGKSFSRSDKLVRHQRTHTGEKPYKCPECGKSFSQAGHLASHQRT |
| | | HTGEKPYKCPECGKSFSRNDALTEHQRTHTGEKPYKCPECGKSFS |
| | | TSGNLTEHQRTHTGEKPYKCPECGKSFSRSDHLTNHQRTHTGKKT |
| | | SASGSGGGSGGDAKSLTAWSRTLVTFKDVFVDFTREEWKLLDTAQ |
| | | QILYRNVMLENYKNLVSLGYQLTKPDVILRLEKGEEPWLVEREIH |
| | | QETHPDSETAFEIKSSVSGGKRPAATKKAGQAKKKKGSYPYDVPD |
| | | YA* |
| 153 | ZF35-KRAB | MAPKKKRKVGIHGVPAAGSSGSLEPGEKPYKCPECGKSFSSKKAL |
| | (MR-32104) | TEHQRTHTGEKPYKCPECGKSFSTSHSLTEHQRTHTGEKPYKCPE |
| | | CGKSFSHKNALQNHQRTHTGEKPYKCPECGKSFSQSSNLVRHQRT |
| | | HTGEKPYKCPECGKSFSRSDHLTTHQRTHTGEKPYKCPECGKSFS |
| | | TKNSLTEHQRTHTGEKPYKCPECGKSFSTTGNLTVHQRTHTGKKT |
| | | SASGSGGGSGGDAKSLTAWSRTLVTFKDVFVDFTREEWKLLDTAQ |
| | | QILYRNVMLENYKNLVSLGYQLTKPDVILRLEKGEEPWLVEREIH |
| | | QETHPDSETAFEIKSSVSGGKRPAATKKAGQAKKKKGSYPYDVPD |
| | | YA* |
| 154 | ZF36-KRAB | MAPKKKRKVGIHGVPAAGSSGSLEPGEKPYKCPECGKSFSTSGNL |
| | (MR-32105) | VRHQRTHTGEKPYKCPECGKSFSRADNLTEHQRTHTGEKPYKCPE |
| | | CGKSFSQSGDLRRHQRTHTGEKPYKCPECGKSFSHKNALQNHQRT |
| | | HTGEKPYKCPECGKSFSDPGNLVRHQRTHTGEKPYKCPECGKSFS |
| | | QRANLRAHQRTHTGEKPYKCPECGKSFSDCRDLARHQRTHTGKKT |
| | | SASGSGGGSGGDAKSLTAWSRTLVTFKDVFVDFTREEWKLLDTAQ |
| | | QILYRNVMLENYKNLVSLGYQLTKPDVILRLEKGEEPWLVEREIH |
| | | QETHPDSETAFEIKSSVSGGKRPAATKKAGQAKKKKGSYPYDVPD |
| | | YA* |
| 155 | ZF37-KRAB | MAPKKKRKVGIHGVPAAGSSGSLEPGEKPYKCPECGKSFSRNDAL |
| | (MR-32106) | TEHQRTHTGEKPYKCPECGKSFSDKKDLTRHQRTHTGEKPYKCPE |
| | | CGKSFSRADNLTEHQRTHTGEKPYKCPECGKSFSHKNALQNHQRT |
| | | HTGEKPYKCPECGKSFSTSHSLTEHQRTHTGEKPYKCPECGKSFS |
| | | TSGNLVRHQRTHTGEKPYKCPECGKSFSREDNLHTHQRTHTGKKT |
| | | SASGSGGGSGGDAKSLTAWSRTLVTFKDVFVDFTREEWKLLDTAQ |
| | | QILYRNVMLENYKNLVSLGYQLTKPDVILRLEKGEEPWLVEREIH |
| | | QETHPDSETAFEIKSSVSGGKRPAATKKAGQAKKKKGSYPYDVPD |
| | | YA* |
| 156 | ZF38-KRAB | MAPKKKRKVGIHGVPAAGSSGSLEPGEKPYKCPECGKSFSRKDNL |
| | (MR-32107) | KNHQRTHTGEKPYKCPECGKSFSRADNLTEHQRTHTGEKPYKCPE |
| | | CGKSFSDPGALVRHQRTHTGEKPYKCPECGKSFSDKKDLTRHQRT |
| | | HTGEKPYKCPECGKSFSDPGALVRHQRTHTGEKPYKCPECGKSFS |
| | | RSDNLVRHQRTHTGEKPYKCPECGKSFSSPADLTRHQRTHTGKKT |
| | | SASGSGGGSGGDAKSLTAWSRTLVTFKDVFVDFTREEWKLLDTAQ |
| | | QILYRNVMLENYKNLVSLGYQLTKPDVILRLEKGEEPWLVEREIH |
| | | QETHPDSETAFEIKSSVSGGKRPAATKKAGQAKKKKGSYPYDVPD |
| | | YA* |
| 157 | ZF39-KRAB | MAPKKKRKVGIHGVPAAGSSGSLEPGEKPYKCPECGKSFSRSDEL |
| | (MR-32108) | VRHQRTHTGEKPYKCPECGKSFSQSGDLRRHQRTHTGEKPYKCPE |
| | | CGKSFSQRANLRAHQRTHTGEKPYKCPECGKSFSTHLDLIRHQRT |
| | | HTGEKPYKCPECGKSFSRSDHLTTHQRTHTGEKPYKCPECGKSFS |
| | | TSGNLTEHQRTHTGEKPYKCPECGKSFSQSGDLRRHQRTHTGKKT |
| | | SASGSGGGSGGDAKSLTAWSRTLVTFKDVFVDFTREEWKLLDTAQ |
| | | QILYRNVMLENYKNLVSLGYQLTKPDVILRLEKGEEPWLVEREIH |
| | | QETHPDSETAFEIKSSVSGGKRPAATKKAGQAKKKKGSYPYDVPD |
| | | YA* |
| 158 | ZF40-KRAB | MAPKKKRKVGIHGVPAAGSSGSLEPGEKPYKCPECGKSFSRNDAL |
| | (MR-32109) | TEHQRTHTGEKPYKCPECGKSFSRRDELNVHQRTHTGEKPYKCPE |
| | | CGKSFSRSDELVRHQRTHTGEKPYKCPECGKSFSRADNLTEHQRT |
| | | HTGEKPYKCPECGKSFSTSHSLTEHQRTHTGEKPYKCPECGKSFS |
| | | ERSHLREHQRTHTGEKPYKCPECGKSFSTHLDLIRHQRTHTGKKT |
| | | SASGSGGGSGGDAKSLTAWSRTLVTFKDVFVDFTREEWKLLDTAQ |
| | | QILYRNVMLENYKNLVSLGYQLTKPDVILRLEKGEEPWLVEREIH |
| | | QETHPDSETAFEIKSSVSGGKRPAATKKAGQAKKKKGSYPYDVPD |
| | | YA* |
| 159 | ZF41-KRAB | MAPKKKRKVGIHGVPAAGSSGSLEPGEKPYKCPECGKSFSTSGNL |
| | (MR-32110) | TEHQRTHTGEKPYKCPECGKSFSQSSSLVRHQRTHTGEKPYKCPE |
| | | CGKSFSRSDHLTTHQRTHTGEKPYKCPECGKSFSRSDHLTTHQRT |
| | | HTGEKPYKCPECGKSFSQSSSLVRHQRTHTGEKPYKCPECGKSFS |
| | | SKKALTEHQRTHTGEKPYKCPECGKSFSTKNSLTEHQRTHTGKKT |
| | | SASGSGGGSGGDAKSLTAWSRTLVTFKDVFVDFTREEWKLLDTAQ |
| | | QILYRNVMLENYKNLVSLGYQLTKPDVILRLEKGEEPWLVEREIH |
| | | QETHPDSETAFEIKSSVSGGKRPAATKKAGQAKKKKGSYPYDVPD |
| | | YA* |
| 160 | ZF42-KRAB | MAPKKKRKVGIHGVPAAGSSGSLEPGEKPYKCPECGKSFSTHLDL |
| | (MR-32111) | IRHQRTHTGEKPYKCPECGKSFSDPGNLVRHQRTHTGEKPYKCPE |
| | | CGKSFSRKDNLKNHQRTHTGEKPYKCPECGKSFSDKKDLTRHQRT |
| | | HTGEKPYKCPECGKSFSRSDELVRHQRTHTGEKPYKCPECGKSFS |
| | | THLDLIRHQRTHTGEKPYKCPECGKSFSQSSSLVRHQRTHTGKKT |
| | | SASGSGGGSGGDAKSLTAWSRTLVTFKDVFVDFTREEWKLLDTAQ |
| | | QILYRNVMLENYKNLVSLGYQLTKPDVILRLEKGEEPWLVEREIH |
| | | QETHPDSETAFEIKSSVSGGKRPAATKKAGQAKKKKGSYPYDVPD |
| | | YA* |
| 161 | ZF43-KRAB | MAPKKKRKVGIHGVPAAGSSGSLEPGEKPYKCPECGKSFSTSHSL |
| | (MR-32112) | TEHQRTHTGEKPYKCPECGKSFSQAGHLASHQRTHTGEKPYKCPE |
| | | CGKSFSTKNSLTEHQRTHTGEKPYKCPECGKSFSDCRDLARHQRT |
| | | HTGEKPYKCPECGKSFSQAGHLASHQRTHTGEKPYKCPECGKSFS |
| | | QSSSLVRHQRTHTGEKPYKCPECGKSFSRSDHLTTHQRTHTGKKT |
| | | SASGSGGGSGGDAKSLTAWSRTLVTFKDVFVDFTREEWKLLDTAQ |
| | | QILYRNVMLENYKNLVSLGYQLTKPDVILRLEKGEEPWLVEREIH |
| | | QETHPDSETAFEIKSSVSGGKRPAATKKAGQAKKKKGSYPYDVPD |
| | | YA* |
| 164 | ZF44-KRAB | MAPKKKRKVGGSGGSLEPGEKPYKCPECGKSFSDSGNLRVH |
| | (MR-32694) | QRTHTGEKPYKCPECGKSFSQLAHLRAHQRTHTGEKPYKCP |
| | | ECGKSFSTKNSLTEHQRTHTGEKPYKCPECGKSFSTSGSLV |
| | | RHQRTHTGEKPYKCPECGKSFSSPADLTRHQRTHTGEKPYK |
| | | CPECGKSFSQSSSLVRHQRTHTGEKPYKCPECGKSFSQSGD |
| | | LRRHQRTHTGKKTSASGSGGGSGGDAKSLTAWSRTLVTFKD |
| | | VFVDFTREEWKLLDTAQQILYRNVMLENYKNLVSLGYQLTK |
| | | PDVILRLEKGEEPWLVEREIHQETHPDSETAFEIKSSVSGG |
| | | KRPAATKKAGQAKKKKGSYPYDVPDYA* |
| 165 | ZF45-KRAB | MAPKKKRKVGGSGGSLEPGEKPYKCPECGKSFSRSDNLVRH |
| | (MR-32695) | QRTHTGEKPYKCPECGKSFSQNSTLTEHQRTHTGEKPYKCP |
| | | ECGKSFSQSSSLVRHQRTHTGEKPYKCPECGKSFSQKSSLI |
| | | AHQRTHTGEKPYKCPECGKSFSTSGNLVRHQRTHTGEKPYK |
| | | CPECGKSFSSPADLTRHQRTHTGEKPYKCPECGKSFSRSDE |
| | | LVRHQRTHTGKKTSASGSGGGSGGDAKSLTAWSRTLVTFKD |
| | | VFVDFTREEWKLLDTAQQILYRNVMLENYKNLVSLGYQLTK |
| | | PDVILRLEKGEEPWLVEREIHQETHPDSETAFEIKSSVSGG |
| | | KRPAATKKAGQAKKKKGSYPYDVPDYA* |
| 166 | ZF46-KRAB | MAPKKKRKVGGSGGSLEPGEKPYKCPECGKSFSTSGSLVRH |
| | (MR-32696) | QRTHTGEKPYKCPECGKSFSQRANLRAHQRTHTGEKPYKCP |
| | | ECGKSFSRTDTLRDHQRTHTGEKPYKCPECGKSFSRRDELN |
| | | VHQRTHTGEKPYKCPECGKSFSHRTTLTNHQRTHTGEKPYK |
| | | CPECGKSFSRSDKLTEHQRTHTGEKPYKCPECGKSFSQKSS |
| | | LIAHQRTHTGKKTSASGSGGGSGGDAKSLTAWSRTLVTFKD |
| | | VFVDFTREEWKLLDTAQQILYRNVMLENYKNLVSLGYQLTK |
| | | PDVILRLEKGEEPWLVEREIHQETHPDSETAFEIKSSVSGG |
| | | KRPAATKKAGQAKKKKGSYPYDVPDYA* |
| 167 | ZF47-KRAB | MAPKKKRKVGGSGGSLEPGEKPYKCPECGKSFSRADNLTEH |
| | (MR-32697) | QRTHTGEKPYKCPECGKSFSRSDHLTTHQRTHTGEKPYKCP |
| | | ECGKSFSRSDNLVRHQRTHTGEKPYKCPECGKSFSQNSTLT |
| | | EHQRTHTGEKPYKCPECGKSFSQSSSLVRHQRTHTGEKPYK |
| | | CPECGKSFSQKSSLIAHQRTHTGEKPYKCPECGKSFSTSGN |
| | | LVRHQRTHTGKKTSASGSGGGSGGDAKSLTAWSRTLVTFKD |
| | | VFVDFTREEWKLLDTAQQILYRNVMLENYKNLVSLGYQLTK |
| | | PDVILRLEKGEEPWLVEREIHQETHPDSETAFEIKSSVSGG |
| | | KRPAATKKAGQAKKKKGSYPYDVPDYA* |
| 168 | ZF48-KRAB | MAPKKKRKVGGSGGSLEPGEKPYKCPECGKSFSRSDHLTTH |
| | (MR-32698) | QRTHTGEKPYKCPECGKSFSDPGHLVRHQRTHTGEKPYKCP |
| | | ECGKSFSHKNALQNHQRTHTGEKPYKCPECGKSFSRRDELN |
| | | VHQRTHTGEKPYKCPECGKSFSQRANLRAHQRTHTGEKPYK |
| | | CPECGKSFSTTGNLTVHQRTHTGEKPYKCPECGKSFSSKKH |
| | | LAEHQRTHTGKKTSASGSGGGSGGDAKSLTAWSRTLVTFKD |
| | | VFVDFTREEWKLLDTAQQILYRNVMLENYKNLVSLGYQLTK |
| | | PDVILRLEKGEEPWLVEREIHQETHPDSETAFEIKSSVSGG |
| | | KRPAATKKAGQAKKKKGSYPYDVPDYA* |
| 169 | ZF49-KRAB | MAPKKKRKVGGSGGSLEPGEKPYKCPECGKSFSSKKALTEH |
| | (MR-32699) | QRTHTGEKPYKCPECGKSFSTSGNLTEHQRTHTGEKPYKCP |
| | | ECGKSFSHRTTLTNHQRTHTGEKPYKCPECGKSFSRNDALT |
| | | EHQRTHTGEKPYKCPECGKSFSDSGNLRVHQRTHTGEKPYK |
| | | CPECGKSFSQSGDLRRHQRTHTGEKPYKCPECGKSFSRSDH |
| | | LTNHQRTHTGKKTSASGSGGGSGGDAKSLTAWSRTLVTFKD |
| | | VFVDFTREEWKLLDTAQQILYRNVMLENYKNLVSLGYQLTK |
| | | PDVILRLEKGEEPWLVEREIHQETHPDSETAFEIKSSVSGG |
| | | KRPAATKKAGQAKKKKGSYPYDVPDYA* |

In some embodiments, an expression repressor comprises an amino acid sequence of SEQ ID NO: 152, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 153, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 154, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 155, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 156, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 157, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 158, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 159, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 160, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 161, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto.

In some embodiments, an expression repressor comprises an amino acid sequence of SEQ ID NO: 164 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 165 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 166 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 167 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 168 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 169 or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto.

In some embodiments, an expression repressor comprises a targeting moiety (e.g., a TAL domain, e.g., a TAL domain of Table 13), and an effector moiety comprising KRAB, e.g., a KRAB domain. In some embodiments, an expression repressor is encoded by a nucleic acid sequence of Table 18 (e.g., a nucleic acid sequence of any one of SEQ ID NOs: 284-291). In some embodiments, an expression repressor is encoded by a nucleic acid sequence of Table 18, e.g., a nucleic acid sequence of any one of SEQ ID NOs: 284-291, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, a nucleic acid described herein has a sequence set out in Table 18, or a sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

It is understood that, when provided as an mRNA, a sequence described herein may comprise an RNA sequence in which each position indicated as a T in Table 18 is occupied by a U. In some embodiments, the 3′ poly-A sequence shown in a sequence of Table 18 is omitted. In some embodiment, a 3′ poly-A sequence is included in the nucleic acid, wherein the 3′ poly-A sequence is up to the length shown in a sequence of Table 18.

TABLE 18
Exemplary expression repressor encoding mRNA

| SEQ ID | | |
|---|---|---|
| NO | Description | Sequence |
| 284 | TAL06-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32712) | GGCTCGCATATGGTGGACCTGAGAACCCTTGGATACTCCCAGC |
| | | AGCAGCAGGAGAAGATTAAGCCCAAGGTCCGGTCGACCGTCGC |
| | | TCAACACCACGAGGCCCTCGTGGGACATGGCTTCACCCACGCA |
| | | CACATTGTCGCGCTGTCCCAACACCCTGCCGCCCTTGGAACTG |
| | | TGGCCGTCAAGTACCAGGATATGATCGCCGCTCTGCCTGAAGC |
| | | GACGCATGAAGCGATTGTGGGCGTGGGCAAGAGAGGAGCAGGA |
| | | GCCCGAGCACTGGAAGCCCTTCTGACCGTCGCCGGCGAACTGA |
| | | GGGGTCCACCTCTGCAACTGGATACCGGGCAGCTGCTGAAGAT |
| | | CGCTAAGAGAGGCGGAGTGACCGCCGTCGAAGCCGTCCATGCA |
| | | TGGCGCAACGCGCTGACTGGAGCACCGCTGAACCTGACCCCCG |
| | | AACAAGTCGTTGCCATTGCTAGTAACGGTGGCGGCAAGCAGGC |
| | | TCTGGAAACCGTCCAGCGCCTCCTTCCTGTCCTGTGTCAAGCG |
| | | CACGGGCTCACACCTGAACAAGTAGTGGCCATCGCTTCCAACA |
| | | TTGGTGGCAAGCAGGCCCTGGAAACTGTACAGCGGTTGTTGCC |
| | | TGTCCTCTGCCAAGCGCATGGGCTTACCCCGGAGCAAGTGGTC |
| | | GCTATCGCTTCTCATGATGGTGGGAAGCAGGCGCTGGAAACAG |
| | | TGCAGAGACTGCTTCCGGTGCTGTGCCAGGCACATGGCCTGAC |
| | | GCCGGAACAAGTGGTGGCGATTGCATCTAACGGAGGCGGGAAG |
| | | CAGGCGCTGGAGACAGTGCAACGGCTGCTCCCTGTTCTATGCC |
| | | AAGCTCATGGACTCACCCCCGAGCAAGTCGTCGCCATTGCGTC |
| | | CAACAATGGAGGCAAACAGGCACTGGAAACGGTGCAAAGACTG |
| | | TTGCCTGTACTGTGCCAGGCTCATGGCCTTACTCCCGAACAGG |
| | | TGGTGGCAATTGCGAGTAACATCGGCGGAAAGCAGGCTTTGGA |
| | | AACTGTGCAGCGCTTGCTGCCTGTGCTCTGTCAGGCTCACGGA |
| | | CTCACCCCCGAGCAAGTCGTAGCCATCGCCTCAAATATCGGAG |
| | | GCAAGCAAGCGCTCGAGACTGTGCAGAGGCTCCTGCCTGTGTT |
| | | ATGTCAGGCACACGGGCTGACCCCTGAACAAGTCGTGGCCATT |
| | | GCCTCCAACAACGGCGGAAAACAGGCGCTCGAAACAGTCCAGA |
| | | GGCTTTTGCCCGTGCTGTGTCAAGCTCACGGCCTGACTCCGGA |
| | | GCAAGTGGTCGCCATTGCATCCCATGATGGCGGTAAACAAGCA |
| | | TTGGAAACGGTCCAACGCCTGCTGCCGGTCCTGTGCCAGGCCC |
| | | ATGGTCTGACTCCCGAACAGGTCGTGGCTATCGCCTCCAACGG |
| | | AGGTGGAAAACAAGCCCTGGAGACTGTACAGAGGCTGCTGCCC |
| | | GTGCTATGTCAAGCACATGGACTTACGCCTGAACAAGTGGTGG |
| | | CAATTGCATCACACGACGGAGGGAAGCAAGCCCTTGAAACCGT |
| | | GCAAAGGCTCCTCCCGGTGCTGTGCCAAGCCCATGGATTGACT |
| | | CCAGAACAGGTGGTCGCTATCGCATCACATGACGGTGGAAAGC |
| | | AAGCCCTCGAAACTGTGCAGCGGCTCCTCCCTGTGTTGTGCCA |
| | | GGCGCACGGTCTGACTCCCGAGCAAGTCGTGGCAATCGCCTCG |
| | | AACATCGGGGGCAAACAAGCTCTCGAAACTGTTCAAAGACTGC |
| | | TCCCCGTGCTCTGTCAAGCTCACGGCCTCACTCCTGAACAGGT |
| | | AGTCGCGATTGCCTCCCATGACGGGGGAAAGCAGGCCTTGGAG |
| | | ACAGTCCAGCGGCTCTTGCCGGTCTTGTGCCAAGCACACGGAT |
| | | TGACTCCAGAACAGGTGGTCGCAATCGCCAGCAATATCGGGGG |
| | | GAAGCAAGCCTTGGAAACCGTTCAGCGGCTACTCCCTGTGCTG |
| | | TGTCAAGCCCACGGTCTGACCCCAGAACAAGTGGTGGCTATCG |
| | | CGTCGAACATTGGAGGCAAACAGGCCCTCGAGACTGTCCAGAG |
| | | ACTCCTGCCAGTCCTCTGCCAGGCTCATGGGCTGACTCCTGAG |
| | | CAAGTTGTGGCCATAGCATCCAACGGCGGAGGAAAGCAAGCTC |
| | | TGGAAACCGTACAGAGACTTCTCCCGGTGTTGTGTCAGGCCCA |
| | | CGGACTTACTCCGGAACAGGTTGTCGCCATCGCGTCCAACGGT |
| | | GGTGGCAGGCCTGCTCTGGAATCCATCGTGGCACAGTTGTCAA |
| | | GACCAGATCCTGCCCTGGCTGCGCTCACCAACGACCACTTGGT |
| | | GGCTCTGGCCTGCCTTGGCGGTCGCCCTGCCTTGGATGCCGTG |
| | | AAGAAGGGACTTCCTCACGCCCCGGCACTGATTAAGCGCACCA |
| | | ATCGCAGGATCCCTGAACGGACCTCGCACAGGGTCGCTTCCGG |
| | | ATCCGGTGGAGGATCAGGCGGCGATGCGAAGTCCCTCACCGCT |
| | | TGGTCACGGACCCTAGTGACCTTCAAAGACGTGTTCGTGGATT |
| | | TCACCAGGGAAGAATGGAAGCTCCTGGACACCGCCCAGCAAAT |
| | | ACTGTACCGGAACGTCATGCTGGAGAACTATAAGAACCTGGTC |
| | | AGCTTGGGCTACCAGCTCACCAAGCCGGACGTGATCCTGCGGC |
| | | TGGAGAAGGGAGAGGAGCCTTGGCTCGTGGAAAGGGAAATTCA |
| | | TCAGGAGACTCACCCTGACAGCGAAACCGCCTTCGAGATTAAG |
| | | TCCTCCGTGTCCGGAGGAAAGAGGCCTGCCGCTACCAAGAAAG |
| | | CAGGACAAGCCAAGAAGAAGAAGGGATCCTACCCGTATGACGT |
| | | GCCCGACTACGCCTAATGAGCGGCCGCTTAATTAAGCTGCCTT |
| | | CTGCGGGGCTTGCCTTCTGGCCATGCCCTTCTTCTCTCCCTTG |
| | | CACCTGTACCTCTTGGTCTTTGAATAAAGCCTGAGTAGGAAGT |
| | | CTAGAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAA |
| 285 | TAL07-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32713) | GGCTCGCATATGGTGGACCTGAGAACCCTTGGATACTCCCAGC |
| | | AGCAGCAGGAGAAGATTAAGCCCAAGGTCCGGTCGACCGTCGC |
| | | TCAACACCACGAGGCCCTCGTGGGACATGGCTTCACCCACGCA |
| | | CACATTGTCGCGCTGTCCCAACACCCTGCCGCCCTTGGAACTG |
| | | TGGCCGTCAAGTACCAGGATATGATCGCCGCTCTGCCTGAAGC |
| | | GACGCATGAAGCGATTGTGGGCGTGGGCAAGAGAGGAGCAGGA |
| | | GCCCGAGCACTGGAAGCCCTTCTGACCGTCGCCGGCGAACTGA |
| | | GGGGTCCACCTCTGCAACTGGATACCGGGCAGCTGCTGAAGAT |
| | | CGCTAAGAGAGGCGGAGTGACCGCCGTCGAAGCCGTCCATGCA |
| | | TGGCGCAACGCGCTGACTGGAGCACCGCTGAACCTGACCCCCG |
| | | AACAAGTCGTTGCCATTGCTAGTAACATTGGCGGCAAGCAGGC |
| | | TCTGGAAACCGTCCAGCGCCTCCTTCCTGTCCTGTGTCAAGCG |
| | | CACGGGCTCACACCTGAACAAGTAGTGGCCATCGCTTCCCATG |
| | | ATGGTGGCAAGCAGGCCCTGGAAACTGTACAGCGGTTGTTGCC |
| | | TGTCCTCTGCCAAGCGCATGGGCTTACCCCGGAGCAAGTGGTC |
| | | GCTATCGCTTCTAACGGCGGTGGGAAGCAGGCGCTGGAAACAG |
| | | TGCAGAGACTGCTTCCGGTGCTGTGCCAGGCACATGGCCTGAC |
| | | GCCGGAACAAGTGGTGGCGATTGCATCTCACGATGGCGGGAAG |
| | | CAGGCGCTGGAGACAGTGCAACGGCTGCTCCCTGTTCTATGCC |
| | | AAGCTCATGGACTCACCCCCGAGCAAGTCGTCGCCATTGCGTC |
| | | CCACGATGGAGGCAAACAGGCACTGGAAACGGTGCAAAGACTG |
| | | TTGCCTGTACTGTGCCAGGCTCATGGCCTTACTCCCGAACAGG |
| | | TGGTGGCAATTGCGAGTAACAACGGCGGAAAGCAGGCTTTGGA |
| | | AACTGTGCAGCGCTTGCTGCCTGTGCTCTGTCAGGCTCACGGA |
| | | CTCACCCCCGAGCAAGTCGTAGCCATCGCCTCAAATGGAGGAG |
| | | GCAAGCAAGCGCTCGAGACTGTGCAGAGGCTCCTGCCTGTGTT |
| | | ATGTCAGGCACACGGGCTGACCCCTGAACAAGTCGTGGCCATT |
| | | GCCTCCAACATCGGCGGAAAACAGGCGCTCGAAACAGTCCAGA |
| | | GGCTTTTGCCCGTGCTGTGTCAAGCTCACGGCCTGACTCCGGA |
| | | GCAAGTGGTCGCCATTGCATCCAACGGAGGCGGTAAACAAGCA |
| | | TTGGAAACGGTCCAACGCCTGCTGCCGGTCCTGTGCCAGGCCC |
| | | ATGGTCTGACTCCCGAACAGGTCGTGGCTATCGCCTCCAACGG |
| | | AGGTGGAAAACAAGCCCTGGAGACTGTACAGAGGCTGCTGCCC |
| | | GTGCTATGTCAAGCACATGGACTTACGCCTGAACAAGTGGTGG |
| | | CAATTGCATCAAACGGCGGAGGGAAGCAAGCCCTTGAAACCGT |
| | | GCAAAGGCTCCTCCCGGTGCTGTGCCAAGCCCATGGATTGACT |
| | | CCAGAACAGGTGGTCGCTATCGCATCAAACAATGGTGGAAAGC |
| | | AAGCCCTCGAAACTGTGCAGCGGCTCCTCCCTGTGTTGTGCCA |
| | | GGCGCACGGTCTGACTCCCGAGCAAGTCGTGGCAATCGCCTCG |
| | | AACATCGGTGGGAAGCAAGCTCTGGAGACTGTGCAGAGGCTCC |
| | | TGCCGGTGCTTTGTCAGGCCCACGGGCTTACTCCGGAACAAGT |
| | | GGTGGCCATCGCCTCCAACGGGGGGGGCAAACAGGCCTTGGAG |
| | | ACAGTCCAGCGGCTCTTGCCGGTCTTGTGCCAAGCACACGGAT |
| | | TGACTCCAGAACAGGTGGTCGCAATCGCCAGCAATATCGGGGG |
| | | GAAGCAAGCCTTGGAAACCGTTCAGCGGCTACTCCCTGTGCTG |
| | | TGTCAAGCCCACGGTCTGACCCCAGAACAAGTGGTGGCTATCG |
| | | CGTCGAACATTGGAGGCAAACAGGCCCTCGAGACTGTCCAGAG |
| | | ACTCCTGCCAGTCCTCTGCCAGGCTCATGGGCTGACTCCTGAG |
| | | CAAGTTGTGGCCATAGCATCCAACAACGGAGGAAAGCAAGCTC |
| | | TGGAAACCGTACAGAGACTTCTCCCGGTGTTGTGTCAGGCCCA |
| | | CGGACTTACTCCGGAACAGGTTGTCGCCATCGCGTCCAACAAC |
| | | GGTGGCAGGCCTGCTCTGGAATCCATCGTGGCACAGTTGTCAA |
| | | GACCAGATCCTGCCCTGGCTGCGCTCACCAACGACCACTTGGT |
| | | GGCTCTGGCCTGCCTTGGCGGTCGCCCTGCCTTGGATGCCGTG |
| | | AAGAAGGGACTTCCTCACGCCCCGGCACTGATTAAGCGCACCA |
| | | ATCGCAGGATCCCTGAACGGACCTCGCACAGGGTCGCTTCCGG |
| | | ATCCGGTGGAGGATCAGGCGGCGATGCGAAGTCCCTCACCGCT |
| | | TGGTCACGGACCCTAGTGACCTTCAAAGACGTGTTCGTGGATT |
| | | TCACCAGGGAAGAATGGAAGCTCCTGGACACCGCCCAGCAAAT |
| | | ACTGTACCGGAACGTCATGCTGGAGAACTATAAGAACCTGGTC |
| | | AGCTTGGGCTACCAGCTCACCAAGCCGGACGTGATCCTGCGGC |
| | | TGGAGAAGGGAGAGGAGCCTTGGCTCGTGGAAAGGGAAATTCA |
| | | TCAGGAGACTCACCCTGACAGCGAAACCGCCTTCGAGATTAAG |
| | | TCCTCCGTGTCCGGAGGAAAGAGGCCTGCCGCTACCAAGAAAG |
| | | CAGGACAAGCCAAGAAGAAGAAGGGATCCTACCCGTATGACGT |
| | | GCCCGACTACGCCTAATGAGCGGCCGCTTAATTAAGCTGCCTT |
| | | CTGCGGGGCTTGCCTTCTGGCCATGCCCTTCTTCTCTCCCTTG |
| | | CACCTGTACCTCTTGGTCTTTGAATAAAGCCTGAGTAGGAAGT |
| | | CTAGAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAA |
| 286 | TAL08-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32714) | GGCTCGCATATGGTGGACCTGAGAACCCTTGGATACTCCCAGC |
| | | AGCAGCAGGAGAAGATTAAGCCCAAGGTCCGGTCGACCGTCGC |
| | | TCAACACCACGAGGCCCTCGTGGGACATGGCTTCACCCACGCA |
| | | CACATTGTCGCGCTGTCCCAACACCCTGCCGCCCTTGGAACTG |
| | | TGGCCGTCAAGTACCAGGATATGATCGCCGCTCTGCCTGAAGC |
| | | GACGCATGAAGCGATTGTGGGCGTGGGCAAGAGAGGAGCAGGA |
| | | GCCCGAGCACTGGAAGCCCTTCTGACCGTCGCCGGCGAACTGA |
| | | GGGGTCCACCTCTGCAACTGGATACCGGGCAGCTGCTGAAGAT |
| | | CGCTAAGAGAGGCGGAGTGACCGCCGTCGAAGCCGTCCATGCA |
| | | TGGCGCAACGCGCTGACTGGAGCACCGCTGAACCTGACCCCCG |
| | | AACAAGTCGTTGCCATTGCTAGTAACAACGGCGGCAAGCAGGC |
| | | TCTGGAAACCGTCCAGCGCCTCCTTCCTGTCCTGTGTCAAGCG |
| | | CACGGGCTCACACCTGAACAAGTAGTGGCCATCGCTTCCAACA |
| | | TTGGTGGCAAGCAGGCCCTGGAAACTGTACAGCGGTTGTTGCC |
| | | TGTCCTCTGCCAAGCGCATGGGCTTACCCCGGAGCAAGTGGTC |
| | | GCTATCGCTTCTAACAACGGTGGGAAGCAGGCGCTGGAAACAG |
| | | TGCAGAGACTGCTTCCGGTGCTGTGCCAGGCACATGGCCTGAC |
| | | GCCGGAACAAGTGGTGGCGATTGCATCTAACGGAGGCGGGAAG |
| | | CAGGCGCTGGAGACAGTGCAACGGCTGCTCCCTGTTCTATGCC |
| | | AAGCTCATGGACTCACCCCCGAGCAAGTCGTCGCCATTGCGTC |
| | | CCACGATGGCGGAAAGCAGGCTTTGGAAACTGTGCAGCGCTTG |
| | | CTGCCTGTGCTCTGTCAGGCTCACGGACTCACCCCCGAGCAAG |
| | | TCGTAGCCATCGCCTCAAATATCGGAGGCAAGCAAGCGCTCGA |
| | | GACTGTGCAGAGGCTCCTGCCTGTGTTATGTCAGGCACACGGG |
| | | CTGACCCCTGAACAAGTCGTGGCCATTGCCTCCAACGGTGGCG |
| | | GAAAACAGGCGCTCGAAACAGTCCAGAGGCTTTTGCCCGTGCT |
| | | GTGTCAAGCTCACGGCCTGACTCCGGAGCAAGTGGTCGCCATT |
| | | GCATCCCATGATGGCGGTAAACAAGCATTGGAAACGGTCCAAC |
| | | GCCTGCTGCCGGTCCTGTGCCAGGCCCATGGTCTGACTCCCGA |
| | | ACAGGTCGTGGCTATCGCCTCCAACATCGGTGGAAAACAAGCC |
| | | CTGGAGACTGTACAGAGGCTGCTGCCCGTGCTATGTCAAGCAC |
| | | ATGGACTTACGCCTGAACAAGTGGTGGCAATTGCATCACACGA |
| | | CGGAGGGAAGCAAGCCCTTGAAACCGTGCAACGACTTCTGCCC |
| | | GTCCTGTGTCAGGCACACGGTCTGACTCCCGAACAGGTGGTGG |
| | | CCATCGCCAGCAATATCGGGGGCAAGCAGGCGTTGGAAACTGT |
| | | GCAAAGGCTCCTCCCGGTGCTGTGCCAAGCCCATGGATTGACT |
| | | CCAGAACAGGTGGTCGCTATCGCATCACATGACGGTGGAAAGC |
| | | AAGCCCTCGAAACTGTGCAGCGGCTCCTCCCTGTGTTGTGCCA |
| | | GGCGCACGGTCTGACTCCCGAGCAAGTCGTGGCAATCGCCTCG |
| | | AACGGAGGGGGCAAACAAGCTCTCGAAACTGTTCAAAGACTGC |
| | | TCCCCGTGCTCTGTCAAGCTCACGGCCTCACTCCTGAACAGGT |
| | | AGTCGCGATTGCCTCCAACGGGGGGGGAAAGCAGGCCTTGGAG |
| | | ACAGTCCAGCGGCTCTTGCCGGTCTTGTGCCAAGCACACGGAT |
| | | TGACTCCAGAACAGGTGGTCGCAATCGCCAGCCACGACGGGGG |
| | | GAAGCAAGCCTTGGAAACCGTTCAGCGGCTACTCCCTGTGCTG |
| | | TGTCAAGCCCACGGTCTGACCCCAGAACAAGTGGTGGCTATCG |
| | | CGTCGCACGATGGAGGCAAACAGGCCCTCGAGACTGTCCAGAG |
| | | ACTCCTGCCAGTCCTCTGCCAGGCTCATGGGCTGACTCCTGAG |
| | | CAAGTTGTGGCCATAGCATCCAACGGCGGAGGAAAGCAAGCTC |
| | | TGGAAACCGTACAGAGACTTCTCCCGGTGTTGTGTCAGGCCCA |
| | | CGGACTTACTCCGGAACAGGTTGTCGCCATCGCGTCCAACATA |
| | | GGTGGCAGGCCTGCTCTGGAATCCATCGTGGCACAGTTGTCAA |
| | | GACCAGATCCTGCCCTGGCTGCGCTCACCAACGACCACTTGGT |
| | | GGCTCTGGCCTGCCTTGGCGGTCGCCCTGCCTTGGATGCCGTG |
| | | AAGAAGGGACTTCCTCACGCCCCGGCACTGATTAAGCGCACCA |
| | | ATCGCAGGATCCCTGAACGGACCTCGCACAGGGTCGCTTCCGG |
| | | ATCCGGTGGAGGATCAGGCGGCGATGCGAAGTCCCTCACCGCT |
| | | TGGTCACGGACCCTAGTGACCTTCAAAGACGTGTTCGTGGATT |
| | | TCACCAGGGAAGAATGGAAGCTCCTGGACACCGCCCAGCAAAT |
| | | ACTGTACCGGAACGTCATGCTGGAGAACTATAAGAACCTGGTC |
| | | AGCTTGGGCTACCAGCTCACCAAGCCGGACGTGATCCTGCGGC |
| | | TGGAGAAGGGAGAGGAGCCTTGGCTCGTGGAAAGGGAAATTCA |
| | | TCAGGAGACTCACCCTGACAGCGAAACCGCCTTCGAGATTAAG |
| | | TCCTCCGTGTCCGGAGGAAAGAGGCCTGCCGCTACCAAGAAAG |
| | | CAGGACAAGCCAAGAAGAAGAAGGGATCCTACCCGTATGACGT |
| | | GCCCGACTACGCCTAATGAGCGGCCGCTTAATTAAGCTGCCTT |
| | | CTGCGGGGCTTGCCTTCTGGCCATGCCCTTCTTCTCTCCCTTG |
| | | CACCTGTACCTCTTGGTCTTTGAATAAAGCCTGAGTAGGAAGT |
| | | CTAGAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAA |
| 287 | TAL09-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32715) | GGCTCGCATATGGTGGACCTGAGAACCCTTGGATACTCCCAGC |
| | | AGCAGCAGGAGAAGATTAAGCCCAAGGTCCGGTCGACCGTCGC |
| | | TCAACACCACGAGGCCCTCGTGGGACATGGCTTCACCCACGCA |
| | | CACATTGTCGCGCTGTCCCAACACCCTGCCGCCCTTGGAACTG |
| | | TGGCCGTCAAGTACCAGGATATGATCGCCGCTCTGCCTGAAGC |
| | | GACGCATGAAGCGATTGTGGGCGTGGGCAAGAGAGGAGCAGGA |
| | | GCCCGAGCACTGGAAGCCCTTCTGACCGTCGCCGGCGAACTGA |
| | | GGGGTCCACCTCTGCAACTGGATACCGGGCAGCTGCTGAAGAT |
| | | CGCTAAGAGAGGCGGAGTGACCGCCGTCGAAGCCGTCCATGCA |
| | | TGGCGCAACGCGCTGACTGGAGCACCGCTGAACCTGACCCCCG |
| | | AACAAGTCGTTGCCATTGCTAGTAACAACGGCGGCAAGCAGGC |
| | | TCTGGAAACCGTCCAGCGCCTCCTTCCTGTCCTGTGTCAAGCG |
| | | CACGGGCTCACACCTGAACAAGTAGTGGCCATCGCTTCCAACA |
| | | TTGGTGGCAAGCAGGCCCTGGAAACTGTACAGCGGTTGTTGCC |
| | | TGTCCTCTGCCAAGCGCATGGGCTTACCCCGGAGCAAGTGGTC |
| | | GCTATCGCTTCTCATGATGGTGGGAAGCAGGCGCTGGAAACAG |
| | | TGCAGAGACTGCTTCCGGTGCTGTGCCAGGCACATGGCCTGAC |
| | | GCCGGAACAAGTGGTGGCGATTGCATCTAACGGAGGCGGGAAG |
| | | CAGGCGCTGGAGACAGTGCAACGGCTGCTCCCTGTTCTATGCC |
| | | AAGCTCATGGACTCACCCCCGAGCAAGTCGTCGCCATTGCGTC |
| | | CCACGACGGAGGCAAACAGGCACTGGAAACGGTGCAAAGACTG |
| | | TTGCCTGTACTGTGCCAGGCTCATGGCCTTACTCCCGAACAGG |
| | | TGGTGGCAATTGCGAGTAACATCGGCGGAAAGCAGGCTTTGGA |
| | | AACTGTGCAGCGCTTGCTGCCTGTGCTCTGTCAGGCTCACGGA |
| | | CTCACCCCCGAGCAAGTCGTAGCCATCGCCTCAAATAACGGAG |
| | | GCAAGCAAGCGCTCGAGACTGTGCAGAGGCTCCTGCCTGTGTT |
| | | ATGTCAGGCACACGGGCTGACCCCTGAACAAGTCGTGGCCATT |
| | | GCCTCCAACAACGGCGGAAAACAGGCGCTCGAAACAGTCCAGA |
| | | GGCTTTTGCCCGTGCTGTGTCAAGCTCACGGCCTGACTCCGGA |
| | | GCAAGTGGTCGCCATTGCATCCAATGGCGGCGGTAAACAAGCA |
| | | TTGGAAACGGTCCAACGCCTGCTGCCGGTCCTGTGCCAGGCCC |
| | | ATGGTCTGACTCCCGAACAGGTCGTGGCTATCGCCTCCAACGG |
| | | AGGTGGAAAACAAGCCCTGGAGACTGTACAGAGGCTGCTGCCC |
| | | GTGCTATGTCAAGCACATGGACTTACGCCTGAACAAGTGGTGG |
| | | CAATTGCATCAAACGGGGGAGGGAAGCAAGCCCTTGAAACCGT |
| | | GCAAAGGCTCCTCCCGGTGCTGTGCCAAGCCCATGGATTGACT |
| | | CCAGAACAGGTGGTCGCTATCGCATCAAACAACGGTGGAAAGC |
| | | AAGCCCTCGAAACTGTGCAGCGGCTCCTCCCTGTGTTGTGCCA |
| | | GGCGCACGGTCTGACTCCCGAGCAAGTCGTGGCAATCGCCTCG |
| | | CACGATGGGGGCAAACAAGCTCTCGAAACTGTTCAAAGACTGC |
| | | TCCCCGTGCTCTGTCAAGCTCACGGCCTCACTCCTGAACAGGT |
| | | AGTCGCGATTGCCTCCCATGACGGGGGAAAGCAGGCCTTGGAG |
| | | ACAGTCCAGCGGCTCTTGCCGGTCTTGTGCCAAGCACACGGAT |
| | | TGACTCCAGAACAGGTGGTCGCAATCGCCAGCCATGATGGGGG |
| | | GAAGCAAGCCTTGGAAACCGTTCAGCGGCTACTCCCTGTGCTG |
| | | TGTCAAGCCCACGGTCTGACCCCAGAACAAGTGGTGGCTATCG |
| | | CGTCGAACGGAGGAGGCAAACAGGCCCTCGAGACTGTCCAGAG |
| | | ACTCCTGCCAGTCCTCTGCCAGGCTCATGGGCTGACTCCTGAG |
| | | CAAGTTGTGGCCATAGCATCCAACAATGGAGGAAAGCAAGCTC |
| | | TGGAAACCGTACAGAGACTTCTCCCGGTGTTGTGTCAGGCCCA |
| | | CGGACTTACTCCGGAACAGGTTGTCGCCATCGCGTCCAACATC |
| | | GGTGGCAGGCCTGCTCTGGAATCCATCGTGGCACAGTTGTCAA |
| | | GACCAGATCCTGCCCTGGCTGCGCTCACCAACGACCACTTGGT |
| | | GGCTCTGGCCTGCCTTGGCGGTCGCCCTGCCTTGGATGCCGTG |
| | | AAGAAGGGACTTCCTCACGCCCCGGCACTGATTAAGCGCACCA |
| | | ATCGCAGGATCCCTGAACGGACCTCGCACAGGGTCGCTTCCGG |
| | | ATCCGGTGGAGGATCAGGCGGCGATGCGAAGTCCCTCACCGCT |
| | | TGGTCACGGACCCTAGTGACCTTCAAAGACGTGTTCGTGGATT |
| | | TCACCAGGGAAGAATGGAAGCTCCTGGACACCGCCCAGCAAAT |
| | | ACTGTACCGGAACGTCATGCTGGAGAACTATAAGAACCTGGTC |
| | | AGCTTGGGCTACCAGCTCACCAAGCCGGACGTGATCCTGCGGC |
| | | TGGAGAAGGGAGAGGAGCCTTGGCTCGTGGAAAGGGAAATTCA |
| | | TCAGGAGACTCACCCTGACAGCGAAACCGCCTTCGAGATTAAG |
| | | TCCTCCGTGTCCGGAGGAAAGAGGCCTGCCGCTACCAAGAAAG |
| | | CAGGACAAGCCAAGAAGAAGAAGGGATCCTACCCGTATGACGT |
| | | GCCCGACTACGCCTAATGAGCGGCCGCTTAATTAAGCTGCCTT |
| | | CTGCGGGGCTTGCCTTCTGGCCATGCCCTTCTTCTCTCCCTTG |
| | | CACCTGTACCTCTTGGTCTTTGAATAAAGCCTGAGTAGGAAGT |
| | | CTAGAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAA |
| 288 | TAL10-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32913) | GGCTCGCATATGGTGGACCTGAGAACCCTTGGATACTCCCAGC |
| | | AGCAGCAGGAGAAGATTAAGCCCAAGGTCCGGTCGACCGTCGC |
| | | TCAACACCACGAGGCCCTCGTGGGACATGGCTTCACCCACGCA |
| | | CACATTGTCGCGCTGTCCCAACACCCTGCCGCCCTTGGAACTG |
| | | TGGCCGTCAAGTACCAGGATATGATCGCCGCTCTGCCTGAAGC |
| | | GACGCATGAAGCGATTGTGGGCGTGGGCAAGAGAGGAGCAGGA |
| | | GCCCGAGCACTGGAAGCCCTTCTGACCGTCGCCGGCGAACTGA |
| | | GGGGTCCACCTCTGCAACTGGATACCGGGCAGCTGCTGAAGAT |
| | | CGCTAAGAGAGGCGGAGTGACCGCCGTCGAAGCCGTCCATGCA |
| | | TGGCGCAACGCGCTGACTGGAGCACCGCTGAACCTCACCCCGG |
| | | AACAGGTGGTGGCGATTGCTTCCCACGACGGTGGCAAGCAGGC |
| | | CTTGGAAACCGTCCAACGGCTTTTGCCGGTGCTTTGCCAGGCC |
| | | CATGGACTGACCCCCGAACAAGTCGTTGCCATTGCTAGTCACG |
| | | ACGGCGGCAAGCAGGCTCTGGAAACCGTCCAGCGCCTCCTTCC |
| | | TGTCCTGTGTCAAGCGCACGGGCTCACACCTGAACAAGTAGTG |
| | | GCCATCGCTTCCAACGGCGGTGGCAAGCAGGCCCTGGAAACTG |
| | | TACAGCGGTTGTTGCCTGTCCTCTGCCAAGCGCATGGGCTTAC |
| | | CCCGGAGCAAGTGGTCGCTATCGCTTCTAATAATGGTGGGAAG |
| | | CAGGCGCTGGAAACAGTGCAGAGACTGCTTCCGGTGCTGTGCC |
| | | AGGCACATGGCCTGACGCCGGAACAAGTGGTGGCGATTGCATC |
| | | TAACATTGGCGGGAAGCAGGCGCTGGAGACAGTGCAACGGCTG |
| | | CTCCCTGTTCTATGCCAAGCTCATGGACTCACCCCCGAGCAAG |
| | | TCGTCGCCATTGCGTCCAACAACGGAGGCAAACAGGCACTGGA |
| | | AACGGTGCAAAGACTGTTGCCTGTACTGTGCCAGGCTCATGGC |
| | | CTTACTCCCGAACAGGTGGTGGCAATTGCGAGTAACAACGGTG |
| | | GAAAGCAGGCGCTCGAAACTGTCCAGAGATTGCTGCCTGTGCT |
| | | GTGTCAGGCACACGGACTGACCCCAGAACAAGTGGTGGCCATC |
| | | GCGAGCCATGACGGCGGAAAGCAGGCTTTGGAAACTGTGCAGC |
| | | GCTTGCTGCCTGTGCTCTGTCAGGCTCACGGACTCACCCCCGA |
| | | GCAAGTCGTAGCCATCGCCTCAAATATCGGAGGCAAGCAAGCG |
| | | CTCGAGACTGTGCAGAGGCTCCTGCCTGTGTTATGTCAGGCAC |
| | | ACGGGCTGACCCCTGAACAAGTCGTGGCCATTGCCTCCAACGG |
| | | TGGCGGAAAACAGGCGCTCGAAACAGTCCAGAGGCTTTTGCCC |
| | | GTGCTGTGTCAAGCTCACGGCCTGACTCCGGAGCAAGTGGTCG |
| | | CCATTGCATCCAATAACGGCGGTAAACAAGCATTGGAAACGGT |
| | | CCAACGCCTGCTGCCGGTCCTGTGCCAGGCCCATGGTCTGACT |
| | | CCCGAACAGGTCGTGGCTATCGCCTCCAACGGAGGTGGAAAAC |
| | | AAGCCCTGGAGACTGTACAGAGGCTGCTGCCCGTGCTATGTCA |
| | | AGCACATGGACTTACGCCTGAACAAGTGGTGGCAATTGCATCA |
| | | CACGACGGAGGGAAGCAAGCCCTTGAAACCGTGCAAAGGCTCC |
| | | TCCCGGTGCTGTGCCAAGCCCATGGATTGACTCCAGAACAGGT |
| | | GGTCGCTATCGCATCAAACATCGGTGGAAAGCAAGCCCTCGAA |
| | | ACTGTGCAGCGGCTCCTCCCTGTGTTGTGCCAGGCGCACGGTC |
| | | TGACTCCCGAGCAAGTCGTGGCAATCGCCTCGAACAATGGGGG |
| | | CAAACAAGCTCTCGAAACTGTTCAAAGACTGCTCCCCGTGCTC |
| | | TGTCAAGCTCACGGCCTCACTCCTGAACAGGTAGTCGCGATTG |
| | | CCTCCCATGACGGGGGAAAGCAGGCCTTGGAGACAGTCCAGCG |
| | | GCTCTTGCCGGTCTTGTGCCAAGCACACGGATTGACTCCAGAA |
| | | CAGGTGGTCGCAATCGCCAGCCATGATGGAGGAAAGCAAGCTC |
| | | TGGAAACCGTACAGAGACTTCTCCCGGTGTTGTGTCAGGCCCA |
| | | CGGACTTACTCCGGAACAGGTTGTCGCCATCGCGTCCAACATC |
| | | GGTGGCAGGCCTGCTCTGGAATCCATCGTGGCACAGTTGTCAA |
| | | GACCAGATCCTGCCCTGGCTGCGCTCACCAACGACCACTTGGT |
| | | GGCTCTGGCCTGCCTTGGCGGTCGCCCTGCCTTGGATGCCGTG |
| | | AAGAAGGGACTTCCTCACGCCCCGGCACTGATTAAGCGCACCA |
| | | ATCGCAGGATCCCTGAACGGACCTCGCACAGGGTCGCTTCCGG |
| | | ATCCGGTGGAGGATCAGGCGGCGATGCGAAGTCCCTCACCGCT |
| | | TGGTCACGGACCCTAGTGACCTTCAAAGACGTGTTCGTGGATT |
| | | TCACCAGGGAAGAATGGAAGCTCCTGGACACCGCCCAGCAAAT |
| | | ACTGTACCGGAACGTCATGCTGGAGAACTATAAGAACCTGGTC |
| | | AGCTTGGGCTACCAGCTCACCAAGCCGGACGTGATCCTGCGGC |
| | | TGGAGAAGGGAGAGGAGCCTTGGCTCGTGGAAAGGGAAATTCA |
| | | TCAGGAGACTCACCCTGACAGCGAAACCGCCTTCGAGATTAAG |
| | | TCCTCCGTGTCCGGAGGAAAGAGGCCTGCCGCTACCAAGAAAG |
| | | CAGGACAAGCCAAGAAGAAGAAGGGATCCTACCCGTATGACGT |
| | | GCCCGACTACGCCTAATGAGCGGCCGCTTAATTAAGCTGCCTT |
| | | CTGCGGGGCTTGCCTTCTGGCCATGCCCTTCTTCTCTCCCTTG |
| | | CACCTGTACCTCTTGGTCTTTGAATAAAGCCTGAGTAGGAAGT |
| | | CTAGAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAA |
| 289 | TAL11-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32914) | GGCTCGCATATGGTGGACCTGAGAACCCTTGGATACTCCCAGC |
| | | AGCAGCAGGAGAAGATTAAGCCCAAGGTCCGGTCGACCGTCGC |
| | | TCAACACCACGAGGCCCTCGTGGGACATGGCTTCACCCACGCA |
| | | CACATTGTCGCGCTGTCCCAACACCCTGCCGCCCTTGGAACTG |
| | | TGGCCGTCAAGTACCAGGATATGATCGCCGCTCTGCCTGAAGC |
| | | GACGCATGAAGCGATTGTGGGCGTGGGCAAGAGAGGAGCAGGA |
| | | GCCCGAGCACTGGAAGCCCTTCTGACCGTCGCCGGCGAACTGA |
| | | GGGGTCCACCTCTGCAACTGGATACCGGGCAGCTGCTGAAGAT |
| | | CGCTAAGAGAGGCGGAGTGACCGCCGTCGAAGCCGTCCATGCA |
| | | TGGCGCAACGCGCTGACTGGAGCACCGCTGAACCTGACCCCCG |
| | | AACAAGTCGTTGCCATTGCTAGTCATGACGGCGGCAAGCAGGC |
| | | TCTGGAAACCGTCCAGCGCCTCCTTCCTGTCCTGTGTCAAGCG |
| | | CACGGGCTCACACCTGAACAAGTAGTGGCCATCGCTTCCAACG |
| | | GAGGTGGCAAGCAGGCCCTGGAAACTGTACAGCGGTTGTTGCC |
| | | TGTCCTCTGCCAAGCGCATGGGCTTACCCCGGAGCAAGTGGTC |
| | | GCTATCGCTTCTAACAACGGAGGCAAGCAAGCGCTCGAGACTG |
| | | TGCAGAGGCTCCTGCCTGTGTTATGTCAGGCACACGGGCTGAC |
| | | CCCTGAACAAGTCGTGGCCATTGCCTCCAACATCGGCGGAAAA |
| | | CAGGCGCTCGAAACAGTCCAGAGGCTTTTGCCCGTGCTGTGTC |
| | | AAGCTCACGGCCTGACTCCGGAGCAAGTGGTCGCCATTGCATC |
| | | CAACAACGGCGGTAAACAAGCATTGGAAACGGTCCAACGCCTG |
| | | CTGCCGGTCCTGTGCCAGGCCCATGGTCTGACTCCCGAACAGG |
| | | TCGTGGCTATCGCCTCCAACGGAGGTGGTAAGCAAGCACTGGA |
| | | AACCGTGCAGCGGTTGCTCCCCGTGCTCTGTCAGGCTCACGGG |
| | | CTCACTCCAGAGCAGGTGGTGGCCATTGCCAGCCATGACGGTG |
| | | GAAAACAAGCCCTGGAGACTGTACAGAGGCTGCTGCCCGTGCT |
| | | ATGTCAAGCACATGGACTTACGCCTGAACAAGTGGTGGCAATT |
| | | GCATCAAACATTGGAGGGAAGCAAGCCCTTGAAACCGTGCAAC |
| | | GACTTCTGCCCGTCCTGTGTCAGGCACACGGTCTGACTCCCGA |
| | | ACAGGTGGTGGCCATCGCCAGCCACGACGGGGGCAAGCAGGCG |
| | | TTGGAAACTGTGCAAAGGCTCCTCCCGGTGCTGTGCCAAGCCC |
| | | ATGGATTGACTCCAGAACAGGTGGTCGCTATCGCATCACATGA |
| | | CGGTGGAAAGCAAGCCCTCGAAACTGTGCAGCGGCTCCTCCCT |
| | | GTGTTGTGCCAGGCGCACGGTCTGACTCCCGAGCAAGTCGTGG |
| | | CAATCGCCTCGAACGGAGGGGGCAAACAAGCTCTCGAAACTGT |
| | | TCAAAGACTCTTGCCGGTCTTGTGCCAAGCACACGGATTGACT |
| | | CCAGAACAGGTGGTCGCAATCGCCAGCCACGACGGCGGGAAGC |
| | | AGGCACTCGAAACTGTCCAGCGTCTGCTGCCTGTCCTCTGCCA |
| | | AGCCCACGGCCTCACTCCAGAACAGGTGGTCGCGATTGCAAGC |
| | | AACGGCGGGGGGAAGCAAGCCTTGGAAACCGTTCAGCGGCTAC |
| | | TCCCTGTGCTGTGTCAAGCCCACGGTCTGACCCCAGAACAAGT |
| | | GGTGGCTATCGCGTCGCACGATGGAGGCAAACAGGCCCTCGAG |
| | | ACTGTCCAGAGACTCCTGCCAGTCCTCTGCCAGGCTCATGGGC |
| | | TGACTCCTGAGCAAGTTGTGGCCATAGCATCCAACGGCGGCGG |
| | | CAAGCAAGCCCTGGAGACTGTGCAACGGCTGTTGCCGGTCCTG |
| | | TGCCAGGCCCATGGTCTTACGCCGGAACAGGTCGTGGCCATCG |
| | | CGTCAAACAACGGTGGCAAGCAGGCTCTGGAGACAGTGCAACG |
| | | CCTTCTGCCTGTGCTTTGCCAGGCCCACGGCTTGACCCCCGAG |
| | | CAAGTGGTGGCCATCGCCTCGAATAACGGAGGAAAGCAAGCTC |
| | | TGGAAACCGTACAGAGACTTCTCCCGGTGTTGTGTCAGGCCCA |
| | | CGGACTTACTCCGGAACAGGTTGTCGCCATCGCGTCCAACATA |
| | | GGTGGCAGGCCTGCTCTGGAATCCATCGTGGCACAGTTGTCAA |
| | | GACCAGATCCTGCCCTGGCTGCGCTCACCAACGACCACTTGGT |
| | | GGCTCTGGCCTGCCTTGGCGGTCGCCCTGCCTTGGATGCCGTG |
| | | AAGAAGGGACTTCCTCACGCCCCGGCACTGATTAAGCGCACCA |
| | | ATCGCAGGATCCCTGAACGGACCTCGCACAGGGTCGCTTCCGG |
| | | ATCCGGTGGAGGATCAGGCGGCGATGCGAAGTCCCTCACCGCT |
| | | TGGTCACGGACCCTAGTGACCTTCAAAGACGTGTTCGTGGATT |
| | | TCACCAGGGAAGAATGGAAGCTCCTGGACACCGCCCAGCAAAT |
| | | ACTGTACCGGAACGTCATGCTGGAGAACTATAAGAACCTGGTC |
| | | AGCTTGGGCTACCAGCTCACCAAGCCGGACGTGATCCTGCGGC |
| | | TGGAGAAGGGAGAGGAGCCTTGGCTCGTGGAAAGGGAAATTCA |
| | | TCAGGAGACTCACCCTGACAGCGAAACCGCCTTCGAGATTAAG |
| | | TCCTCCGTGTCCGGAGGAAAGAGGCCTGCCGCTACCAAGAAAG |
| | | CAGGACAAGCCAAGAAGAAGAAGGGATCCTACCCGTATGACGT |
| | | GCCCGACTACGCCTAATGAGCGGCCGCTTAATTAAGCTGCCTT |
| | | CTGCGGGGCTTGCCTTCTGGCCATGCCCTTCTTCTCTCCCTTG |
| | | CACCTGTACCTCTTGGTCTTTGAATAAAGCCTGAGTAGGAAGT |
| | | CTAGAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAA |
| 290 | TAL12-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32915) | GGCTCGCATATGGTGGACCTGAGAACCCTTGGATACTCCCAGC |
| | | AGCAGCAGGAGAAGATTAAGCCCAAGGTCCGGTCGACCGTCGC |
| | | TCAACACCACGAGGCCCTCGTGGGACATGGCTTCACCCACGCA |
| | | CACATTGTCGCGCTGTCCCAACACCCTGCCGCCCTTGGAACTG |
| | | TGGCCGTCAAGTACCAGGATATGATCGCCGCTCTGCCTGAAGC |
| | | GACGCATGAAGCGATTGTGGGCGTGGGCAAGAGAGGAGCAGGA |
| | | GCCCGAGCACTGGAAGCCCTTCTGACCGTCGCCGGCGAACTGA |
| | | GGGGTCCACCTCTGCAACTGGATACCGGGCAGCTGCTGAAGAT |
| | | CGCTAAGAGAGGCGGAGTGACCGCCGTCGAAGCCGTCCATGCA |
| | | TGGCGCAACGCGCTGACTGGAGCACCGCTGAACCTGACCCCCG |
| | | AACAAGTCGTTGCCATTGCTAGTCATGACGGCGGCAAGCAGGC |
| | | TCTGGAAACCGTCCAGCGCCTCCTTCCTGTCCTGTGTCAAGCG |
| | | CACGGGCTCACACCTGAACAAGTAGTGGCCATCGCTTCCCATG |
| | | ATGGTGGCAAGCAGGCCCTGGAAACTGTACAGCGGTTGTTGCC |
| | | TGTCCTCTGCCAAGCGCATGGGCTTACCCCGGAGCAAGTGGTC |
| | | GCTATCGCTTCTAACGGCGGCGGCAAACAGGCACTGGAAACGG |
| | | TGCAAAGACTGTTGCCTGTACTGTGCCAGGCTCATGGCCTTAC |
| | | TCCCGAACAGGTGGTGGCAATTGCGAGTAACAACGGCGGAAAG |
| | | CAGGCTTTGGAAACTGTGCAGCGCTTGCTGCCTGTGCTCTGTC |
| | | AGGCTCACGGACTCACCCCCGAGCAAGTCGTAGCCATCGCCTC |
| | | AAATGGAGGAGGCAAGCAAGCGCTCGAGACTGTGCAGAGGCTC |
| | | CTGCCTGTGTTATGTCAGGCACACGGGCTGACCCCTGAACAAG |
| | | TCGTGGCCATTGCCTCCAACAACGGCGGAAAACAGGCGCTCGA |
| | | AACAGTCCAGAGGCTTTTGCCCGTGCTGTGTCAAGCTCACGGC |
| | | CTGACTCCGGAGCAAGTGGTCGCCATTGCATCCCACGACGGCG |
| | | GTAAACAAGCATTGGAAACGGTCCAACGCCTGCTGCCGGTCCT |
| | | GTGCCAGGCCCATGGGCTGACTCCGGAACAAGTCGTCGCGATT |
| | | GCGTCCAACATCGGGGGAAAGCAAGCACTCGAAACAGTGCAGC |
| | | GGCTGCTGCCAGTCCTGTGCCAGGCACATGGCCTGACCCCGGA |
| | | GCAGGTGGTGGCCATCGCGAGCAACATTGGTGGGAAGCAGGCA |
| | | CTGGAAACTGTCCAACGGCTGCTGCCGGTGCTATGCCAGGCCC |
| | | ATGGTCTGACTCCCGAACAGGTCGTGGCTATCGCCTCCAACGG |
| | | AGGTGGAAAACAAGCCCTGGAGACTGTACAGAGGCTGCTGCCC |
| | | GTGCTATGTCAAGCACATGGACTTACGCCTGAACAAGTGGTGG |
| | | CAATTGCATCAAACAACGGAGGGAAGCAAGCCCTTGAAACCGT |
| | | GCAAAGGCTCCTCCCGGTGCTGTGCCAAGCCCATGGATTGACT |
| | | CCAGAACAGGTGGTCGCTATCGCATCAAACGGAGGTGGAAAGC |
| | | AAGCCCTCGAAACTGTGCAGCGGCTCCTCCCTGTGTTGTGCCA |
| | | GGCGCACGGTCTGACTCCCGAGCAAGTCGTGGCAATCGCCTCG |
| | | CACGACGGTGGGAAGCAAGCTCTGGAGACTGTGCAGAGGCTCC |
| | | TGCCGGTGCTTTGTCAGGCCCACGGGCTTACTCCGGAACAAGT |
| | | GGTGGCCATCGCCTCCAACGGGGGGGGCAAACAGGCCTTGGAG |
| | | ACAGTCCAGCGGCTCTTGCCGGTCTTGTGCCAAGCACACGGAT |
| | | TGACTCCAGAACAGGTGGTCGCAATCGCCAGCAATGGCGGGGG |
| | | GAAGCAAGCCTTGGAAACCGTTCAGCGGCTACTCCCTGTGCTG |
| | | TGTCAAGCCCACGGTCTGACCCCAGAACAAGTGGTGGCTATCG |
| | | CGTCGAACGGGGGAGGCAAACAGGCCCTCGAGACTGTCCAGAG |
| | | ACTCCTGCCAGTCCTCTGCCAGGCTCATGGGCTGACTCCTGAG |
| | | CAAGTIGTGGCCATAGCATCCAACAACGGAGGAAAGCAAGCTC |
| | | TGGAAACCGTACAGAGACTTCTCCCGGTGTTGTGTCAGGCCCA |
| | | CGGACTTACTCCGGAACAGGTTGTCGCCATCGCGTCCAACAAC |
| | | GGTGGCAGGCCTGCTCTGGAATCCATCGTGGCACAGTTGTCAA |
| | | GACCAGATCCTGCCCTGGCTGCGCTCACCAACGACCACTTGGT |
| | | GGCTCTGGCCTGCCTTGGCGGTCGCCCTGCCTTGGATGCCGTG |
| | | AAGAAGGGACTTCCTCACGCCCCGGCACTGATTAAGCGCACCA |
| | | ATCGCAGGATCCCTGAACGGACCTCGCACAGGGTCGCTTCCGG |
| | | ATCCGGTGGAGGATCAGGCGGCGATGCGAAGTCCCTCACCGCT |
| | | TGGTCACGGACCCTAGTGACCTTCAAAGACGTGTTCGTGGATT |
| | | TCACCAGGGAAGAATGGAAGCTCCTGGACACCGCCCAGCAAAT |
| | | ACTGTACCGGAACGTCATGCTGGAGAACTATAAGAACCTGGTC |
| | | AGCTTGGGCTACCAGCTCACCAAGCCGGACGTGATCCTGCGGC |
| | | TGGAGAAGGGAGAGGAGCCTTGGCTCGTGGAAAGGGAAATTCA |
| | | TCAGGAGACTCACCCTGACAGCGAAACCGCCTTCGAGATTAAG |
| | | TCCTCCGTGTCCGGAGGAAAGAGGCCTGCCGCTACCAAGAAAG |
| | | CAGGACAAGCCAAGAAGAAGAAGGGATCCTACCCGTATGACGT |
| | | GCCCGACTACGCCTAATGAGCGGCCGCTTAATTAAGCTGCCTT |
| | | CTGCGGGGCTTGCCTTCTGGCCATGCCCTTCTTCTCTCCCTTG |
| | | CACCTGTACCTCTTGGTCTTTGAATAAAGCCTGAGTAGGAAGT |
| | | CTAGAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAA |
| 291 | TAL13-KRAB | AGGAAATAAGAGAGAAAAGAAGAGTAAGAAGAAATATAAGAGC |
| | mRNA (MR- | CACCATGGCCCCAAAGAAGAAGCGGAAAGTCGGAGGTTCAGGC |
| | 32916) | GGCTCGCATATGGTGGACCTGAGAACCCTTGGATACTCCCAGC |
| | | AGCAGCAGGAGAAGATTAAGCCCAAGGTCCGGTCGACCGTCGC |
| | | TCAACACCACGAGGCCCTCGTGGGACATGGCTTCACCCACGCA |
| | | CACATTGTCGCGCTGTCCCAACACCCTGCCGCCCTTGGAACTG |
| | | TGGCCGTCAAGTACCAGGATATGATCGCCGCTCTGCCTGAAGC |
| | | GACGCATGAAGCGATTGTGGGCGTGGGCAAGAGAGGAGCAGGA |
| | | GCCCGAGCACTGGAAGCCCTTCTGACCGTCGCCGGCGAACTGA |
| | | GGGGTCCACCTCTGCAACTGGATACCGGGCAGCTGCTGAAGAT |
| | | CGCTAAGAGAGGCGGAGTGACCGCCGTCGAAGCCGTCCATGCA |
| | | TGGCGCAACGCGCTGACTGGAGCACCGCTGAACCTGACCCCCG |
| | | AACAAGTCGTTGCCATTGCTAGTAACGGTGGCGGCAAGCAGGC |
| | | TCTGGAAACCGTCCAGCGCCTCCTTCCTGTCCTGTGTCAAGCG |
| | | CACGGGCTCACACCTGAACAAGTAGTGGCCATCGCTTCCAACA |
| | | ATGGTGGCAAGCAGGCCCTGGAAACTGTACAGCGGTTGTTGCC |
| | | TGTCCTCTGCCAAGCGCATGGGCTTACCCCGGAGCAAGTGGTC |
| | | GCTATCGCTTCTCATGATGGTGGGAAGCAGGCGCTGGAAACAG |
| | | TGCAGAGACTGCTTCCGGTGCTGTGCCAGGCACATGGCCTGAC |
| | | GCCGGAACAAGTGGTGGCGATTGCATCTCACGATGGCGGGAAG |
| | | CAGGCGCTGGAGACAGTGCAACGGCTGCTCCCTGTTCTATGCC |
| | | AAGCTCATGGACTCACCCCCGAGCAAGTCGTCGCCATTGCGTC |
| | | CAACGGAGGAGGCAAACAGGCACTGGAAACGGTGCAAAGACTG |
| | | TTGCCTGTACTGTGCCAGGCTCATGGCCTTACTCCCGAACAGG |
| | | TGGTGGCAATTGCGAGTCACGACGGCGGAAAGCAGGCTTTGGA |
| | | AACTGTGCAGCGCTTGCTGCCTGTGCTCTGTCAGGCTCACGGA |
| | | CTCACCCCCGAGCAAGTCGTAGCCATCGCCTCAAATATCGGAG |
| | | GCAAGCAAGCGCTCGAGACTGTGCAGAGGCTCCTGCCTGTGTT |
| | | ATGTCAGGCACACGGGCTGACCCCTGAACAAGTCGTGGCCATT |
| | | GCCTCCAACAACGGCGGAAAACAGGCGCTCGAAACAGTCCAGA |
| | | GGCTTTTGCCCGTGCTGTGTCAAGCTCACGGCCTGACTCCGGA |
| | | GCAAGTGGTCGCCATTGCATCCAACAATGGCGGTAAACAAGCA |
| | | TTGGAAACGGTCCAACGCCTGCTGCCGGTCCTGTGCCAGGCCC |
| | | ATGGTCTGACTCCCGAACAGGTCGTGGCTATCGCCTCCAACAT |
| | | CGGAGGGAAGCAAGCCCTTGAAACCGTGCAAAGGCTCCTCCCG |
| | | GTGCTGTGCCAAGCCCATGGATTGACTCCAGAACAGGTGGTCG |
| | | CTATCGCATCAAACAACGGTGGAAAGCAAGCCCTCGAAACTGT |
| | | GCAGCGGCTCCTCCCTGTGTTGTGCCAGGCGCACGGTCTGACT |
| | | CCCGAGCAAGTCGTGGCAATCGCCTCGAACGGAGGGGGCAAAC |
| | | AAGCTCTCGAAACTGTTCAAAGACTGCTCCCCGTGCTCTGTCA |
| | | AGCTCACGGCCTCACTCCTGAACAGGTAGTCGCGATTGCCTCC |
| | | CATGACGGGGGAAAGCAGGCCTTGGAGACAGTCCAGCGGCTCT |
| | | TGCCGGTCTTGTGCCAAGCACACGGATTGACTCCAGAACAGGT |
| | | GGTCGCAATCGCCAGCAATATCGGCGGCAAGCAAGCCCTGGAG |
| | | ACTGTTCAGAGGCTCTTGCCTGTGCTCTGCCAGGCCCATGGCT |
| | | TGACACCTGAACAGGTGGTGGCCATCGCGAGCAACGGTGGGGG |
| | | GAAGCAAGCCTTGGAAACCGTTCAGCGGCTACTCCCTGTGCTG |
| | | TGTCAAGCCCACGGTCTGACCCCAGAACAAGTGGTGGCTATCG |
| | | CGTCGAACAACGGAGGCAAACAGGCCCTCGAGACTGTCCAGAG |
| | | ACTCCTGCCAGTCCTCTGCCAGGCTCATGGGCTGACTCCTGAG |
| | | CAAGTTGTGGCCATAGCATCCAACAACGGAGGAAAGCAAGCTC |
| | | TGGAAACCGTACAGAGACTTCTCCCGGTGTTGTGTCAGGCCCA |
| | | CGGACTTACTCCGGAACAGGTTGTCGCCATCGCGTCCAACAAC |
| | | GGTGGCAGGCCTGCTCTGGAATCCATCGTGGCACAGTTGTCAA |
| | | GACCAGATCCTGCCCTGGCTGCGCTCACCAACGACCACTTGGT |
| | | GGCTCTGGCCTGCCTTGGCGGTCGCCCTGCCTTGGATGCCGTG |
| | | AAGAAGGGACTTCCTCACGCCCCGGCACTGATTAAGCGCACCA |
| | | ATCGCAGGATCCCTGAACGGACCTCGCACAGGGTCGCTTCCGG |
| | | ATCCGGTGGAGGATCAGGCGGCGATGCGAAGTCCCTCACCGCT |
| | | TGGTCACGGACCCTAGTGACCTTCAAAGACGTGTTCGTGGATT |
| | | TCACCAGGGAAGAATGGAAGCTCCTGGACACCGCCCAGCAAAT |
| | | ACTGTACCGGAACGTCATGCTGGAGAACTATAAGAACCTGGTC |
| | | AGCTTGGGCTACCAGCTCACCAAGCCGGACGTGATCCTGCGGC |
| | | TGGAGAAGGGAGAGGAGCCTTGGCTCGTGGAAAGGGAAATTCA |
| | | TCAGGAGACTCACCCTGACAGCGAAACCGCCTTCGAGATTAAG |
| | | TCCTCCGTGTCCGGAGGAAAGAGGCCTGCCGCTACCAAGAAAG |
| | | CAGGACAAGCCAAGAAGAAGAAGGGATCCTACCCGTATGACGT |
| | | GCCCGACTACGCCTAATGAGCGGCCGCTTAATTAAGCTGCCTT |
| | | CTGCGGGGCTTGCCTTCTGGCCATGCCCTTCTTCTCTCCCTTG |
| | | CACCTGTACCTCTTGGTCTTTGAATAAAGCCTGAGTAGGAAGT |
| | | CTAGAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| | | AAAAAAAAAAAAAAAAAAAAAAAAAAAAA |

In some embodiments, an expression repressor is encoded by a nucleic acid sequence of SEQ ID NO: 284, or a nucleic acid sequence with at least 80% 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 285, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 286, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 287, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 288, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 289, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 290, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 291, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

In some embodiments, an expression repressor comprises a targeting moiety (e.g., a TAL domain, e.g., a TAL domain of Table 13), and an effector moiety comprising KRAB, e.g., a KRAB domain. In some embodiments, an expression repressor comprises an amino acid sequence of Table 19 (e.g., amino acid sequence of any one of SEQ ID NOs: 260-267). In some embodiments, an expression repressor comprises an amino acid sequence of Table 19, e.g., an amino acid sequence of any one of SEQ ID NOs: 260-267, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto.

TABLE 19
Exemplary expression repressor polypeptide sequences

| SEQ ID | | |
|---|---|---|
| NO | Description | Sequence |
| 260 | TAL06-KRAB | MAPKKKRKVGGSGGSHMVDLRTLGYSQQQQEKIKPKVRSTV |
| | (MR-32712) | AQHHEALVGHGFTHAHIVALSQHPAALGTVAVKYQDMIAAL |
| | | PEATHEAIVGVGKRGAGARALEALLTVAGELRGPPLQLDTG |
| | | QLLKIAKRGGVTAVEAVHAWRNALTGAPLNLTPEQVVAIAS |
| | | NGGGKQALETVQRLLPVLCQAHGLTPEQVVAIASNIGGKQA |
| | | LETVQRLLPVLCQAHGLTPEQVVAIASHDGGKQALETVQRL |
| | | LPVLCQAHGLTPEQVVAIASNGGGKQALETVQRLLPVLCQA |
| | | HGLTPEQVVAIASNNGGKQALETVQRLLPVLCQAHGLTPEQ |
| | | VVAIASNIGGKQALETVQRLLPVLCQAHGLTPEQVVAIASN |
| | | IGGKQALETVQRLLPVLCQAHGLTPEQVVAIASNNGGKQAL |
| | | ETVQRLLPVLCQAHGLTPEQVVAIASHDGGKQALETVQRLL |
| | | PVLCQAHGLTPEQVVAIASNGGGKQALETVQRLLPVLCQAH |
| | | GLTPEQVVAIASHDGGKQALETVQRLLPVLCQAHGLTPEQV |
| | | VAIASHDGGKQALETVQRLLPVLCQAHGLTPEQVVAIASNI |
| | | GGKQALETVQRLLPVLCQAHGLTPEQVVAIASHDGGKQALE |
| | | TVQRLLPVLCQAHGLTPEQVVAIASNIGGKQALETVQRLLP |
| | | VLCQAHGLTPEQVVAIASNIGGKQALETVQRLLPVLCQAHG |
| | | LTPEQVVAIASNGGGKQALETVQRLLPVLCQAHGLTPEQVV |
| | | AIASNGGGRPALESIVAQLSRPDPALAALTNDHLVALACLG |
| | | GRPALDAVKKGLPHAPALIKRTNRRIPERTSHRVASGSGGG |
| | | SGGDAKSLTAWSRTLVTFKDVFVDFTREEWKLLDTAQQILY |
| | | RNVMLENYKNLVSLGYQLTKPDVILRLEKGEEPWLVEREIH |
| | | QETHPDSETAFEIKSSVSGGKRPAATKKAGQAKKKKGSYPY |
| | | DVPDYA* |
| 261 | TAL07-KRAB | MAPKKKRKVGGSGGSHMVDLRTLGYSQQQQEKIKPKVRSTV |
| | (MR-32713) | AQHHEALVGHGFTHAHIVALSQHPAALGTVAVKYQDMIAAL |
| | | PEATHEAIVGVGKRGAGARALEALLTVAGELRGPPLQLDTG |
| | | QLLKIAKRGGVTAVEAVHAWRNALTGAPLNLTPEQVVAIAS |
| | | NIGGKQALETVQRLLPVLCQAHGLTPEQVVAIASHDGGKQA |
| | | LETVQRLLPVLCQAHGLTPEQVVAIASNGGGKQALETVQRL |
| | | LPVLCQAHGLTPEQVVAIASHDGGKQALETVQRLLPVLCQA |
| | | HGLTPEQVVAIASHDGGKQALETVQRLLPVLCQAHGLTPEQ |
| | | VVAIASNNGGKQALETVQRLLPVLCQAHGLTPEQVVAIASN |
| | | GGGKQALETVQRLLPVLCQAHGLTPEQVVAIASNIGGKQAL |
| | | ETVQRLLPVLCQAHGLTPEQVVAIASNGGGKQALETVQRLL |
| | | PVLCQAHGLTPEQVVAIASNGGGKQALETVQRLLPVLCQAH |
| | | GLTPEQVVAIASNGGGKQALETVQRLLPVLCQAHGLTPEQV |
| | | VAIASNNGGKQALETVQRLLPVLCQAHGLTPEQVVAIASNI |
| | | GGKQALETVQRLLPVLCQAHGLTPEQVVAIASNGGGKQALE |
| | | TVQRLLPVLCQAHGLTPEQVVAIASNIGGKQALETVQRLLP |
| | | VLCQAHGLTPEQVVAIASNIGGKQALETVQRLLPVLCQAHG |
| | | LTPEQVVAIASNNGGKQALETVQRLLPVLCQAHGLTPEQVV |
| | | AIASNNGGRPALESIVAQLSRPDPALAALTNDHLVALACLG |
| | | GRPALDAVKKGLPHAPALIKRTNRRIPERTSHRVASGSGGG |
| | | SGGDAKSLTAWSRTLVTFKDVFVDFTREEWKLLDTAQQILY |
| | | RNVMLENYKNLVSLGYQLTKPDVILRLEKGEEPWLVEREIH |
| | | QETHPDSETAFEIKSSVSGGKRPAATKKAGQAKKKKGSYPY |
| | | DVPDYA* |
| 262 | TAL08-KRAB | MAPKKKRKVGGSGGSHMVDLRTLGYSQQQQEKIKPKVRSTV |
| | (MR-32714) | AQHHEALVGHGFTHAHIVALSQHPAALGTVAVKYQDMIAAL |
| | | PEATHEAIVGVGKRGAGARALEALLTVAGELRGPPLQLDTG |
| | | QLLKIAKRGGVTAVEAVHAWRNALTGAPLNLTPEQVVAIAS |
| | | NNGGKQALETVQRLLPVLCQAHGLTPEQVVAIASNIGGKQA |
| | | LETVQRLLPVLCQAHGLTPEQVVAIASNNGGKQALETVQRL |
| | | LPVLCQAHGLTPEQVVAIASNGGGKQALETVQRLLPVLCQA |
| | | HGLTPEQVVAIASHDGGKQALETVQRLLPVLCQAHGLTPEQ |
| | | VVAIASNIGGKQALETVQRLLPVLCQAHGLTPEQVVAIASN |
| | | GGGKQALETVQRLLPVLCQAHGLTPEQVVAIASHDGGKQAL |
| | | ETVQRLLPVLCQAHGLTPEQVVAIASNIGGKQALETVQRLL |
| | | PVLCQAHGLTPEQVVAIASHDGGKQALETVQRLLPVLCQAH |
| | | GLTPEQVVAIASNIGGKQALETVQRLLPVLCQAHGLTPEQV |
| | | VAIASHDGGKQALETVQRLLPVLCQAHGLTPEQVVAIASNG |
| | | GGKQALETVQRLLPVLCQAHGLTPEQVVAIASNGGGKQALE |
| | | TVQRLLPVLCQAHGLTPEQVVAIASHDGGKQALETVQRLLP |
| | | VLCQAHGLTPEQVVAIASHDGGKQALETVQRLLPVLCQAHG |
| | | LTPEQVVAIASNGGGKQALETVQRLLPVLCQAHGLTPEQVV |
| | | AIASNIGGRPALESIVAQLSRPDPALAALTNDHLVALACLG |
| | | GRPALDAVKKGLPHAPALIKRTNRRIPERTSHRVASGSGGG |
| | | SGGDAKSLTAWSRTLVTFKDVFVDFTREEWKLLDTAQQILY |
| | | RNVMLENYKNLVSLGYQLTKPDVILRLEKGEEPWLVEREIH |
| | | QETHPDSETAFEIKSSVSGGKRPAATKKAGQAKKKKGSYPY |
| | | DVPDYA* |
| 263 | TAL09-KRAB | MAPKKKRKVGGSGGSHMVDLRTLGYSQQQQEKIKPKVRSTV |
| | (MR-32715) | AQHHEALVGHGFTHAHIVALSQHPAALGTVAVKYQDMIAAL |
| | | PEATHEAIVGVGKRGAGARALEALLTVAGELRGPPLQLDTG |
| | | QLLKIAKRGGVTAVEAVHAWRNALTGAPLNLTPEQVVAIAS |
| | | NNGGKQALETVQRLLPVLCQAHGLTPEQVVAIASNIGGKQA |
| | | LETVQRLLPVLCQAHGLTPEQVVAIASHDGGKQALETVQRL |
| | | LPVLCQAHGLTPEQVVAIASNGGGKQALETVQRLLPVLCQA |
| | | HGLTPEQVVAIASHDGGKQALETVQRLLPVLCQAHGLTPEQ |
| | | VVAIASNIGGKQALETVQRLLPVLCQAHGLTPEQVVAIASN |
| | | NGGKQALETVQRLLPVLCQAHGLTPEQVVAIASNNGGKQAL |
| | | ETVQRLLPVLCQAHGLTPEQVVAIASNGGGKQALETVQRLL |
| | | PVLCQAHGLTPEQVVAIASNGGGKQALETVQRLLPVLCQAH |
| | | GLTPEQVVAIASNGGGKQALETVQRLLPVLCQAHGLTPEQV |
| | | VAIASNNGGKQALETVQRLLPVLCQAHGLTPEQVVAIASHD |
| | | GGKQALETVQRLLPVLCQAHGLTPEQVVAIASHDGGKQALE |
| | | TVQRLLPVLCQAHGLTPEQVVAIASHDGGKQALETVQRLLP |
| | | VLCQAHGLTPEQVVAIASNGGGKQALETVQRLLPVLCQAHG |
| | | LTPEQVVAIASNNGGKQALETVQRLLPVLCQAHGLTPEQVV |
| | | AIASNIGGRPALESIVAQLSRPDPALAALTNDHLVALACLG |
| | | GRPALDAVKKGLPHAPALIKRTNRRIPERTSHRVASGSGGG |
| | | SGGDAKSLTAWSRTLVTFKDVFVDFTREEWKLLDTAQQILY |
| | | RNVMLENYKNLVSLGYQLTKPDVILRLEKGEEPWLVEREIH |
| | | QETHPDSETAFEIKSSVSGGKRPAATKKAGQAKKKKGSYPY |
| | | DVPDYA* |
| 264 | TAL10-KRAB | MAPKKKRKVGGSGGSHMVDLRTLGYSQQQQEKIKPKVRSTV |
| | (MR-32913) | AQHHEALVGHGFTHAHIVALSQHPAALGTVAVKYQDMIAAL |
| | | PEATHEAIVGVGKRGAGARALEALLTVAGELRGPPLQLDTG |
| | | QLLKIAKRGGVTAVEAVHAWRNALTGAPLNLTPEQVVAIAS |
| | | HDGGKQALETVQRLLPVLCQAHGLTPEQVVAIASHDGGKQA |
| | | LETVQRLLPVLCQAHGLTPEQVVAIASNGGGKQALETVQRL |
| | | LPVLCQAHGLTPEQVVAIASNNGGKQALETVQRLLPVLCQA |
| | | HGLTPEQVVAIASNIGGKQALETVQRLLPVLCQAHGLTPEQ |
| | | VVAIASNNGGKQALETVQRLLPVLCQAHGLTPEQVVAIASN |
| | | NGGKQALETVQRLLPVLCQAHGLTPEQVVAIASHDGGKQAL |
| | | ETVQRLLPVLCQAHGLTPEQVVAIASNIGGKQALETVQRLL |
| | | PVLCQAHGLTPEQVVAIASNGGGKQALETVQRLLPVLCQAH |
| | | GLTPEQVVAIASNNGGKQALETVQRLLPVLCQAHGLTPEQV |
| | | VAIASNGGGKQALETVQRLLPVLCQAHGLTPEQVVAIASHD |
| | | GGKQALETVQRLLPVLCQAHGLTPEQVVAIASNIGGKQALE |
| | | TVQRLLPVLCQAHGLTPEQVVAIASNNGGKQALETVQRLLP |
| | | VLCQAHGLTPEQVVAIASHDGGKQALETVQRLLPVLCQAHG |
| | | LTPEQVVAIASHDGGKQALETVQRLLPVLCQAHGLTPEQVV |
| | | AIASNIGGRPALESIVAQLSRPDPALAALTNDHLVALACLG |
| | | GRPALDAVKKGLPHAPALIKRTNRRIPERTSHRVASGSGGG |
| | | SGGDAKSLTAWSRTLVTFKDVFVDFTREEWKLLDTAQQILY |
| | | RNVMLENYKNLVSLGYQLTKPDVILRLEKGEEPWLVEREIH |
| | | QETHPDSETAFEIKSSVSGGKRPAATKKAGQAKKKKGSYPY |
| | | DVPDYA* |
| 265 | TAL11-KRAB | MAPKKKRKVGGSGGSHMVDLRTLGYSQQQQEKIKPKVRSTV |
| | (MR-32914) | AQHHEALVGHGFTHAHIVALSQHPAALGTVAVKYQDMIAAL |
| | | PEATHEAIVGVGKRGAGARALEALLTVAGELRGPPLQLDTG |
| | | QLLKIAKRGGVTAVEAVHAWRNALTGAPLNLTPEQVVAIAS |
| | | HDGGKQALETVQRLLPVLCQAHGLTPEQVVAIASNGGGKQA |
| | | LETVQRLLPVLCQAHGLTPEQVVAIASNNGGKQALETVQRL |
| | | LPVLCQAHGLTPEQVVAIASNIGGKQALETVQRLLPVLCQA |
| | | HGLTPEQVVAIASNNGGKQALETVQRLLPVLCQAHGLTPEQ |
| | | VVAIASNGGGKQALETVQRLLPVLCQAHGLTPEQVVAIASH |
| | | DGGKQALETVQRLLPVLCQAHGLTPEQVVAIASNIGGKQAL |
| | | ETVQRLLPVLCQAHGLTPEQVVAIASHDGGKQALETVQRLL |
| | | PVLCQAHGLTPEQVVAIASHDGGKQALETVQRLLPVLCQAH |
| | | GLTPEQVVAIASNGGGKQALETVQRLLPVLCQAHGLTPEQV |
| | | VAIASHDGGKQALETVQRLLPVLCQAHGLTPEQVVAIASNG |
| | | GGKQALETVQRLLPVLCQAHGLTPEQVVAIASHDGGKQALE |
| | | TVQRLLPVLCQAHGLTPEQVVAIASNGGGKQALETVQRLLP |
| | | VLCQAHGLTPEQVVAIASNNGGKQALETVQRLLPVLCQAHG |
| | | LTPEQVVAIASNNGGKQALETVQRLLPVLCQAHGLTPEQVV |
| | | AIASNIGGRPALESIVAQLSRPDPALAALTNDHLVALACLG |
| | | GRPALDAVKKGLPHAPALIKRTNRRIPERTSHRVASGSGGG |
| | | SGGDAKSLTAWSRTLVTFKDVFVDFTREEWKLLDTAQQILY |
| | | RNVMLENYKNLVSLGYQLTKPDVILRLEKGEEPWLVEREIH |
| | | QETHPDSETAFEIKSSVSGGKRPAATKKAGQAKKKKGSYPY |
| | | DVPDYA* |
| 266 | TAL12-KRAB | MAPKKKRKVGGSGGSHMVDLRTLGYSQQQQEKIKPKVRSTV |
| | (MR-32915) | AQHHEALVGHGFTHAHIVALSQHPAALGTVAVKYQDMIAAL |
| | | PEATHEAIVGVGKRGAGARALEALLTVAGELRGPPLQLDTG |
| | | QLLKIAKRGGVTAVEAVHAWRNALTGAPLNLTPEQVVAIAS |
| | | HDGGKQALETVQRLLPVLCQAHGLTPEQVVAIASHDGGKQA |
| | | LETVQRLLPVLCQAHGLTPEQVVAIASNGGGKQALETVQRL |
| | | LPVLCQAHGLTPEQVVAIASNNGGKQALETVQRLLPVLCQA |
| | | HGLTPEQVVAIASNGGGKQALETVQRLLPVLCQAHGLTPEQ |
| | | VVAIASNNGGKQALETVQRLLPVLCQAHGLTPEQVVAIASH |
| | | DGGKQALETVQRLLPVLCQAHGLTPEQVVAIASNIGGKQAL |
| | | ETVQRLLPVLCQAHGLTPEQVVAIASNIGGKQALETVQRLL |
| | | PVLCQAHGLTPEQVVAIASNGGGKQALETVQRLLPVLCQAH |
| | | GLTPEQVVAIASNNGGKQALETVQRLLPVLCQAHGLTPEQV |
| | | VAIASNGGGKQALETVQRLLPVLCQAHGLTPEQVVAIASHD |
| | | GGKQALETVQRLLPVLCQAHGLTPEQVVAIASNGGGKQALE |
| | | TVQRLLPVLCQAHGLTPEQVVAIASNGGGKQALETVQRLLP |
| | | VLCQAHGLTPEQVVAIASNGGGKQALETVQRLLPVLCQAHG |
| | | LTPEQVVAIASNNGGKQALETVQRLLPVLCQAHGLTPEQVV |
| | | AIASNNGGRPALESIVAQLSRPDPALAALTNDHLVALACLG |
| | | GRPALDAVKKGLPHAPALIKRTNRRIPERTSHRVASGSGGG |
| | | SGGDAKSLTAWSRTLVTFKDVFVDFTREEWKLLDTAQQILY |
| | | RNVMLENYKNLVSLGYQLTKPDVILRLEKGEEPWLVEREIH |
| | | QETHPDSETAFEIKSSVSGGKRPAATKKAGQAKKKKGSYPY |
| | | DVPDYA* |
| 267 | TAL13-KRAB | MAPKKKRKVGGSGGSHMVDLRTLGYSQQQQEKIKPKVRSTV |
| | (MR-32916) | AQHHEALVGHGFTHAHIVALSQHPAALGTVAVKYQDMIAAL |
| | | PEATHEAIVGVGKRGAGARALEALLTVAGELRGPPLQLDTG |
| | | QLLKIAKRGGVTAVEAVHAWRNALTGAPLNLTPEQVVAIAS |
| | | NGGGKQALETVQRLLPVLCQAHGLTPEQVVAIASNNGGKQA |
| | | LETVQRLLPVLCQAHGLTPEQVVAIASHDGGKQALETVQRL |
| | | LPVLCQAHGLTPEQVVAIASHDGGKQALETVQRLLPVLCQA |
| | | HGLTPEQVVAIASNGGGKQALETVQRLLPVLCQAHGLTPEQ |
| | | VVAIASHDGGKQALETVQRLLPVLCQAHGLTPEQVVAIASN |
| | | IGGKQALETVQRLLPVLCQAHGLTPEQVVAIASNNGGKQAL |
| | | ETVQRLLPVLCQAHGLTPEQVVAIASNNGGKQALETVQRLL |
| | | PVLCQAHGLTPEQVVAIASNIGGKQALETVQRLLPVLCQAH |
| | | GLTPEQVVAIASNNGGKQALETVQRLLPVLCQAHGLTPEQV |
| | | VAIASNGGGKQALETVQRLLPVLCQAHGLTPEQVVAIASHD |
| | | GGKQALETVQRLLPVLCQAHGLTPEQVVAIASNIGGKQALE |
| | | TVQRLLPVLCQAHGLTPEQVVAIASNGGGKQALETVQRLLP |
| | | VLCQAHGLTPEQVVAIASNNGGKQALETVQRLLPVLCQAHG |
| | | LTPEQVVAIASNNGGKQALETVQRLLPVLCQAHGLTPEQVV |
| | | AIASNNGGRPALESIVAQLSRPDPALAALTNDHLVALACLG |
| | | GRPALDAVKKGLPHAPALIKRTNRRIPERTSHRVASGSGGG |
| | | SGGDAKSLTAWSRTLVTFKDVFVDFTREEWKLLDTAQQILY |
| | | RNVMLENYKNLVSLGYQLTKPDVILRLEKGEEPWLVEREIH |
| | | QETHPDSETAFEIKSSVSGGKRPAATKKAGQAKKKKGSYPY |
| | | DVPDYA* |

In some embodiments, an expression repressor comprises an amino acid sequence of SEQ ID NO: 260, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 261, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 262, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 263, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 264, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 265, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 266, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor comprises an amino acid sequence of SEQ ID NO: 267, or an amino acid sequence with at least 80%, 85%, 90%, 95%, 99%, or 100% identity thereto, or an amino acid sequence having no more than 20, 19, 18, 17, 16, 15, 14, 13, 12, 11, 10, 9, 8, 7, 6, 5, 4, 3, 2, or 1 positions of difference thereto.

### Bicistronic Constructs

In some aspects, the disclosure provides a bicistronic construct. The bicistronic construct may comprise a first expression repressor (e.g., a first expression repressor described herein) and a second expression repressor (e.g., a second expression repressor described herein). In some embodiments, the first expression repressor targets E1 and the second expression repressor targets IL-8 promoter.

A bicistronic nucleic acid encoding ZF36-KRAB_tPT2a_TAL06-KRAB (also called MR-32905) is provided in Table 32 below.

TABLE 32
Bicistronic construct and components

| | | SEQ ID |
|---|---|---|
| Description | Sequence | NO |
| ZF36-KRAB_ | MAPKKKRKVGIHGVPAAGSSGSLEPGEKPYKCPECGKSFSTSGNL | 300 |
| tPT2a_TAL06- | VRHQRTHTGEKPYKCPECGKSFSRADNLTEHQRTHTGEKPYKCPE | |
| KRAB | CGKSFSQSGDLRRHQRTHTGEKPYKCPECGKSFSHKNALQNHQRT | |
| Theoretical | HTGEKPYKCPECGKSFSDPGNLVRHQRTHTGEKPYKCPECGKSFS | |
| protein | QRANLRAHQRTHTGEKPYKCPECGKSFSDCRDLARHQRTHTGKKT | |
| sequence | SASGSGGGSGGDAKSLTAWSRTLVTFKDVFVDFTREEWKLLDTAQ | |
| | QILYRNVMLENYKNLVSLGYQLTKPDVILRLEKGEEPWLVEREIH | |
| | QETHPDSETAFEIKSSVSGGKRPAATKKAGQAKKKKGSGSATNFS | |
| | LLKQAGDVEENPGPTSAGKLGSGEGRGSLLTCGDVEENPGPLEGS | |
| | SGSGSPKKKRKVGIHGVPAAGSSGSHMVDLRTLGYSQQQQEKIKP | |
| | KVRSTVAQHHEALVGHGFTHAHIVALSQHPAALGTVAVKYQDMIA | |
| | ALPEATHEAIVGVGKRGAGARALEALLTVAGELRGPPLQLDTGQL | |
| | LKIAKRGGVTAVEAVHAWRNALTGAPLNLTPEQVVAIASNGGGKQ | |
| | ALETVQRLLPVLCQAHGLTPEQVVAIASNIGGKQALETVQRLLPV | |
| | LCQAHGLTPEQVVAIASHDGGKQALETVQRLLPVLCQAHGLTPEQ | |
| | VVAIASNGGGKQALETVQRLLPVLCQAHGLTPEQVVAIASNNGGK | |
| | QALETVQRLLPVLCQAHGLTPEQVVAIASNIGGKQALETVORLLP | |
| | VLCQAHGLTPEQVVAIASNIGGKQALETVQRLLPVLCQAHGLTPE | |
| | QVVAIASNNGGKQALETVQRLLPVLCQAHGLTPEQVVAIASHDGG | |
| | KQALETVQRLLPVLCQAHGLTPEQVVAIASNGGGKQALETVORLL | |
| | PVLCQAHGLTPEQVVAIASHDGGKQALETVQRLLPVLCQAHGLTP | |
| | EQVVAIASHDGGKQALETVQRLLPVLCQAHGLTPEQVVAIASNIG | |
| | GKQALETVQRLLPVLCQAHGLTPEQVVAIASHDGGKQALETVQRL | |
| | LPVLCQAHGLTPEQVVAIASNIGGKQALETVQRLLPVLCQAHGLT | |
| | PEQVVAIASNIGGKQALETVQRLLPVLCQAHGLTPEQVVAIASNG | |
| | GGKQALETVQRLLPVLCQAHGLTPEQVVAIASNGGGRPALESIVA | |
| | QLSRPDPALAALTNDHLVALACLGGRPALDAVKKGLPHAPALIKR | |
| | TNRRIPERTSHRVASGSGGGSGGARDDAKSLTAWSRTLVTFKDVF | |
| | VDFTREEWKLLDTAQQILYRNVMLENYKNLVSLGYQLTKPDVILR | |
| | LEKGEEPWLVEREIHQETHPDSETAFEIKSSVPSSGGKRPAATKK | |
| | AGQAKKKKGSGS** (SEQ ID NO: 300) | |
| ZF36-KRAB_ | GGGAAAUAAGAGAGAAAAGAAGAGUAAGAAGAAAUAUAAGAGCCA | 301 |
| tPT2a_TAL06- | CCAUGGCCCCAAAGAAGAAGAGAAAGGUCGGAAUUCAUGGCGUGC | |
| KRAB mRNA | CCGCAGCCGGCAGCAGCGGUUCCCUUGAGCCGGGAGAGAAACCGU | |
| sequence | ACAAGUGUCCUGAAUGUGGAAAGAGUUUCUCCACCUCGGGCAACC | |
| | UCGUGCGCCACCAGAGGACACACACCGGCGAGAAGCCCUACAAGU | |
| | GCCCCGAGUGCGGUAAAUCCUUUUCACGCGCGGACAAUCUGACUG | |
| | AACACCAGAGAACGCAUACCGGGGAGAAGCCCUACAAGUGCCCGG | |
| | AAUGCGGUAAAAGCUUCUCCCAGUCCGGGGACUUAAGGCGGCAUC | |
| | AAAGAACUCAUACUGGAGAGAAGCCGUACAAAUGCCCUGAAUGCG | |
| | GAAAGUCCUUCUCCCACAAGAACGCCCUGCAGAACCAUCAACGGA | |
| | CACAUACUGGCGAAAAACCAUACAAGUGUCCAGAAUGUGGGAAGU | |
| | CAUUUUCCGACCCGGGGAACCUGGUCCGGCACCAACGCACACACA | |
| | CUGGUGAAAAACCCUACAAAUGUCCGGAGUGUGGGAAGAGUUUUA | |
| | GCCAGAGAGCCAACCUGAGAGCUCAUCAGCGGACCCAUACAGGAG | |
| | AGAAGCCUUACAAAUGCCCCGAAUGUGGAAAAUCAUUCAGCGACU | |
| | GCCGCGACCUUGCUAGGCACCAGCGCACUCAUACCGGAAAAAAGA | |
| | CCUCCGCCAGCGGAUCAGGAGGUGGAUCAGGCGGAGAUGCCAAGU | |
| | CACUGACCGCAUGGUCCCGGACCCUUGUGACCUUCAAGGACGUGU | |
| | UCGUGGACUUCACCAGAGAGGAGUGGAAGCUGCUGGACACCGCGC | |
| | AACAGAUUCUCUACCGGAACGUGAUGCUGGAGAACUACAAGAAUC | |
| | UGGUGUCCCUGGGCUACCAGCUGACCAAGCCGGACGUGAUCCUCC | |
| | GCCUCGAAAAGGGGGAGGAGCCUUGGCUCGUGGAGCGGGAAAUCC | |
| | ACCAGGAGACUCACCCGGACUCCGAGACUGCCUUCGAAAUCAAGU | |
| | CAUCGGUGUCCGGUGGAAAGCGGCCUGCAGCGACUAAGAAGGCCG | |
| | GACAGGCCAAGAAGAAGAAGGGCUCAGGCUCAGCAACCAACUUCU | |
| | CCUUGCUCAAACAAGCCGGGGACGUCGAAGAGAACCCCGGACCGA | |
| | CUAGCGCCGGAAAGCUGGGUUCCGGAGAGGGUCGAGGUUCCCUCC | |
| | UUACCUGUGGAGAUGUGGAAGAAAACCCAGGACCCCUGGAGGGCU | |
| | CCAGCGGUAGCGGAAGCCCGAAGAAGAAGCGGAAGGUCGGAAUCC | |
| | ACGGAGUGCCUGCCGCUGGAAGCUCGGGUUCCCAUAUGGUGGACC | |
| | UGAGAACCCUUGGAUACUCCCAGCAGCAGCAGGAGAAGAUUAAGC | |
| | CCAAGGUCCGGUCGACCGUCGCUCAACACCACGAGGCCCUCGUGG | |
| | GACAUGGCUUCACCCACGCACACAUUGUCGCGCUGUCCCAACACC | |
| | CUGCCGCCCUUGGAACUGUGGCCGUCAAGUACCAGGAUAUGAUCG | |
| | CCGCUCUGCCUGAAGCGACGCAUGAAGCGAUUGUGGGCGUGGGCA | |
| | AGAGAGGAGCAGGAGCCCGAGCACUGGAAGCCCUUCUGACCGUCG | |
| | CCGGCGAACUGAGGGGUCCACCUCUGCAACUGGAUACCGGGCAGC | |
| | UGCUGAAGAUCGCUAAGAGAGGCGGAGUGACCGCCGUCGAAGCCG | |
| | UCCAUGCAUGGCGCAACGCGCUGACUGGAGCACCGCUGAACCUGA | |
| | CCCCCGAACAAGUCGUUGCCAUUGCUAGUAACGGUGGCGGCAAGC | |
| | AGGCUCUGGAAACCGUCCAGCGCCUCCUUCCUGUCCUGUGUCAAG | |
| | CGCACGGGCUCACACCUGAACAAGUAGUGGCCAUCGCUUCCAACA | |
| | UUGGUGGCAAGCAGGCCCUGGAAACUGUACAGCGGUUGUUGCCUG | |
| | UCCUCUGCCAAGCGCAUGGGCUUACCCCGGAGCAAGUGGUCGCUA | |
| | UCGCUUCUCAUGAUGGUGGGAAGCAGGCGCUGGAAACAGUGCAGA | |
| | GACUGCUUCCGGUGCUGUGCCAGGCACAUGGCCUGACGCCGGAAC | |
| | AAGUGGUGGCGAUUGCAUCUAACGGAGGCGGGAAGCAGGCGCUGG | |
| | AGACAGUGCAACGGCUGCUCCCUGUUCUAUGCCAAGCUCAUGGAC | |
| | UCACCCCCGAGCAAGUCGUCGCCAUUGCGUCCAACAAUGGAGGCA | |
| | AACAGGCACUGGAAACGGUGCAAAGACUGUUGCCUGUACUGUGCC | |
| | AGGCUCAUGGCCUUACUCCCGAACAGGUGGUGGCAAUUGCGAGUA | |
| | ACAUCGGCGGAAAGCAGGCUUUGGAAACUGUGCAGCGCUUGCUGC | |
| | CUGUGCUCUGUCAGGCUCACGGACUCACCCCCGAGCAAGUCGUAG | |
| | CCAUCGCCUCAAAUAUCGGAGGCAAGCAAGCGCUCGAGACUGUGC | |
| | AGAGGCUCCUGCCUGUGUUAUGUCAGGCACACGGGCUGACCCCUG | |
| | AACAAGUCGUGGCCAUUGCCUCCAACAACGGCGGAAAACAGGCGC | |
| | UCGAAACAGUCCAGAGGCUUUUGCCCGUGCUGUGUCAAGCUCACG | |
| | GCCUGACUCCGGAGCAAGUGGUCGCCAUUGCAUCCCAUGAUGGCG | |
| | GUAAACAAGCAUUGGAAACGGUCCAACGCCUGCUGCCGGUCCUGU | |
| | GCCAGGCCCAUGGUCUGACUCCCGAACAGGUCGUGGCUAUCGCCU | |
| | CCAACGGAGGUGGAAAACAAGCCCUGGAGACUGUACAGAGGCUGC | |
| | UGCCCGUGCUAUGUCAAGCACAUGGACUUACGCCUGAACAAGUGG | |
| | UGGCAAUUGCAUCACACGACGGAGGGAAGCAAGCCCUUGAAACCG | |
| | UGCAAAGGCUCCUCCCGGUGCUGUGCCAAGCCCAUGGAUUGACUC | |
| | CAGAACAGGUGGUCGCUAUCGCAUCACAUGACGGUGGAAAGCAAG | |
| | CCCUCGAAACUGUGCAGCGGCUCCUCCCUGUGUUGUGCCAGGCGC | |
| | ACGGUCUGACUCCCGAGCAAGUCGUGGCAAUCGCCUCGAACAUCG | |
| | GGGGCAAACAAGCUCUCGAAACUGUUCAAAGACUGCUCCCCGUGC | |
| | UCUGUCAAGCUCACGGCCUCACUCCUGAACAGGUAGUCGCGAUUG | |
| | CCUCCCAUGACGGGGGAAAGCAGGCCUUGGAGACAGUCCAGCGGC | |
| | UCUUGCCGGUCUUGUGCCAAGCACACGGAUUGACUCCAGAACAGG | |
| | UGGUCGCAAUCGCCAGCAAUAUCGGGGGGAAGCAAGCCUUGGAAA | |
| | CCGUUCAGCGGCUACUCCCUGUGCUGUGUCAAGCCCACGGUCUGA | |
| | CCCCAGAACAAGUGGUGGCUAUCGCGUCGAACAUUGGAGGCAAAC | |
| | AGGCCCUCGAGACUGUCCAGAGACUCCUGCCAGUCCUCUGCCAGG | |
| | CUCAUGGGCUGACUCCUGAGCAAGUUGUGGCCAUAGCAUCCAACG | |
| | GCGGAGGAAAGCAAGCUCUGGAAACCGUACAGAGACUUCUCCCGG | |
| | UGUUGUGUCAGGCCCACGGACUUACUCCGGAACAGGUUGUCGCCA | |
| | UCGCGUCCAACGGUGGUGGCAGGCCUGCUCUGGAAUCCAUCGUGG | |
| | CACAGUUGUCAAGACCAGAUCCUGCCCUGGCUGCGCUCACCAACG | |
| | ACCACUUGGUGGCUCUGGCCUGCCUUGGCGGUCGCCCUGCCUUGG | |
| | AUGCCGUGAAGAAGGGACUUCCUCACGCCCCGGCACUGAUUAAGC | |
| | GCACCAAUCGCAGGAUCCCUGAACGGACCUCGCACAGGGUCGCAU | |
| | CCGGUUCAGGAGGAGGGUCCGGAGGAGCUAGGGAUGAUGCCAAGA | |
| | GCUUGACCGCCUGGUCACGGACUCUUGUCACUUUCAAGGAUGUCU | |
| | UUGUGGACUUUACCAGAGAAGAAUGGAAGCUCCUGGACACUGCGC | |
| | AGCAGAUCCUGUACAGAAACGUGAUGUUGGAAAACUACAAAAACC | |
| | UGGUGUCACUCGGGUACCAACUCACCAAGCCCGACGUCAUCCUGC | |
| | GCCUGGAAAAGGGCGAAGAACCGUGGUUGGUCGAGAGAGAAAUCC | |
| | AUCAGGAAACCCACCCUGACUCGGAAACCGCCUUCGAGAUCAAGU | |
| | CCUCGGUGCCUAGCUCCGGAGGAAAAAGGCCCGCCGCAACCAAGA | |
| | AGGCUGGGCAGGCUAAGAAAAAGAAGGGGUCCGGCUCCUAAUGAG | |
| | CGGCCGCUUAAUUAAGCUGCCUUCUGCGGGGCUUGCCUUCUGGCC | |
| | AUGCCCUUCUUCUCUCCCUUGCACCUGUACCUCUUGGUCUUUGAA | |
| | UAAAGCCUGAGUAGGAAGUCUAGAAAAAAAAAAAAAAAAAAAAAA | |
| | AAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAA | |
| DBD Protein | LEPGEKPYKCPECGKSFSTSGNLVRHORTHTGEKPYKCPECGKSF | 114 |
| sequence 1 from | SRADNLTEHQRTHTGEKPYKCPECGKSFSQSGDLRRHQRTHTGEK | |
| bicistronic | PYKCPECGKSFSHKNALQNHQRTHTGEKPYKCPECGKSFSDPGNL | |
| construct | VRHQRTHTGEKPYKCPECGKSFSQRANLRAHQRTHTGEKPYKCPE | |
| | CGKSFSDCRDLARHORTHTGKKTS | |
| DBD RNA | CUUGAGCCGGGAGAGAAACCGUACAAGUGUCCUGAAUGUGGAAAG | 302 |
| sequence 1 from | AGUUUCUCCACCUCGGGCAACCUCGUGCGCCACCAGAGGACACAC | |
| bicistronic | ACCGGCGAGAAGCCCUACAAGUGCCCCGAGUGCGGUAAAUCCUUU | |
| construct | UCACGCGCGGACAAUCUGACUGAACACCAGAGAACGCAUACCGGG | |
| | GAGAAGCCCUACAAGUGCCCGGAAUGCGGUAAAAGCUUCUCCCAG | |
| | UCCGGGGACUUAAGGCGGCAUCAAAGAACUCAUACUGGAGAGAAG | |
| | CCGUACAAAUGCCCUGAAUGCGGAAAGUCCUUCUCCCACAAGAAC | |
| | GCCCUGCAGAACCAUCAACGGACACAUACUGGCGAAAAACCAUAC | |
| | AAGUGUCCAGAAUGUGGGAAGUCAUUUUCCGACCCGGGGAACCUG | |
| | GUCCGGCACCAACGCACACACACUGGUGAAAAACCCUACAAAUGU | |
| | CCGGAGUGUGGGAAGAGUUUUAGCCAGAGAGCCAACCUGAGAGCU | |
| | CAUCAGCGGACCCAUACAGGAGAGAAGCCUUACAAAUGCCCCGAA | |
| | UGUGGAAAAUCAUUCAGCGACUGCCGCGACCUUGCUAGGCACCAG | |
| | CGCACUCAUACCGGAAAAAAGACCUCC | |
| Effector protein | DAKSLTAWSRTLVTFKDVFVDFTREEWKLLDTAQQILYRNVMLEN | 13 |
| sequence from | YKNLVSLGYQLTKPDVILRLEKGEEPWLVEREIHQETHPDSETAF | |
| bicistronic | EIKSSV | |
| construct | | |
| Effector RNA | GAUGCCAAGUCACUGACCGCAUGGUCCCGGACCCUUGUGACCUUC | 303 |
| sequence 1 from | AAGGACGUGUUCGUGGACUUCACCAGAGAGGAGUGGAAGCUGCUG | |
| bicistronic | GACACCGCGCAACAGAUUCUCUACCGGAACGUGAUGCUGGAGAAC | |
| construct | UACAAGAAUCUGGUGUCCCUGGGCUACCAGCUGACCAAGCCGGAC | |
| | GUGAUCCUCCGCCUCGAAAAGGGGGAGGAGCCUUGGCUCGUGGAG | |
| | CGGGAAAUCCACCAGGAGACUCACCCGGACUCCGAGACUGCCUUC | |
| | GAAAUCAAGUCAUCGGUG | |
| DBD Protein | HMVDLRTLGYSQQQQEKIKPKVRSTVAQHHEALVGHGFTHAHIVA | 268 |
| sequence 2 from | LSQHPAALGTVAVKYQDMIAALPEATHEAIVGVGKRGAGARALEA | |
| bicistronic | LLTVAGELRGPPLQLDTGQLLKIAKRGGVTAVEAVHAWRNALTGA | |
| construct | PLNLTPEQVVAIASNGGGKQALETVQRLLPVLCQAHGLTPEQVVA | |
| | IASNIGGKQALETVQRLLPVLCQAHGLTPEQVVAIASHDGGKQAL | |
| | ETVQRLLPVLCQAHGLTPEQVVAIASNGGGKQALETVQRLLPVLC | |
| | QAHGLTPEQVVAIASNNGGKQALETVQRLLPVLCQAHGLTPEQVV | |
| | AIASNIGGKQALETVQRLLPVLCQAHGLTPEQVVAIASNIGGKQA | |
| | LETVQRLLPVLCQAHGLTPEQVVAIASNNGGKQALETVQRLLPVL | |
| | CQAHGLTPEQVVAIASHDGGKQALETVQRLLPVLCQAHGLTPEQV | |
| | VAIASNGGGKQALETVQRLLPVLCQAHGLTPEQVVAIASHDGGKQ | |
| | ALETVQRLLPVLCQAHGLTPEQVVAIASHDGGKQALETVQRLLPV | |
| | LCQAHGLTPEQVVAIASNIGGKQALETVQRLLPVLCQAHGLTPEQ | |
| | VVAIASHDGGKQALETVQRLLPVLCQAHGLTPEQVVAIASNIGGK | |
| | QALETVQRLLPVLCQAHGLTPEQVVAIASNIGGKQALETVQRLLP | |
| | VLCQAHGLTPEQVVAIASNGGGKQALETVQRLLPVLCQAHGLTPE | |
| | QVVAIASNGGGRPALESIVAQLSRPDPALAALTNDHLVALACLGG | |
| | RPALDAVKKGLPHAPALIKRTNRRIPERTSHRV | |
| DBD RNA | CAUAUGGUGGACCUGAGAACCCUUGGAUACUCCCAGCAGCAGCAG | 304 |
| sequence 2 from | GAGAAGAUUAAGCCCAAGGUCCGGUCGACCGUCGCUCAACACCAC | |
| bicistronic | GAGGCCCUCGUGGGACAUGGCUUCACCCACGCACACAUUGUCGCG | |
| construct | CUGUCCCAACACCCUGCCGCCCUUGGAACUGUGGCCGUCAAGUAC | |
| | CAGGAUAUGAUCGCCGCUCUGCCUGAAGCGACGCAUGAAGCGAUU | |
| | GUGGGCGUGGGCAAGAGAGGAGCAGGAGCCCGAGCACUGGAAGCC | |
| | CUUCUGACCGUCGCCGGCGAACUGAGGGGUCCACCUCUGCAACUG | |
| | GAUACCGGGCAGCUGCUGAAGAUCGCUAAGAGAGGCGGAGUGACC | |
| | GCCGUCGAAGCCGUCCAUGCAUGGCGCAACGCGCUGACUGGAGCA | |
| | CCGCUGAACCUGACCCCCGAACAAGUCGUUGCCAUUGCUAGUAAC | |
| | GGUGGCGGCAAGCAGGCUCUGGAAACCGUCCAGCGCCUCCUUCCU | |
| | GUCCUGUGUCAAGCGCACGGGCUCACACCUGAACAAGUAGUGGCC | |
| | AUCGCUUCCAACAUUGGUGGCAAGCAGGCCCUGGAAACUGUACAG | |
| | CGGUUGUUGCCUGUCCUCUGCCAAGCGCAUGGGCUUACCCCGGAG | |
| | CAAGUGGUCGCUAUCGCUUCUCAUGAUGGUGGGAAGCAGGCGCUG | |
| | GAAACAGUGCAGAGACUGCUUCCGGUGCUGUGCCAGGCACAUGGC | |
| | CUGACGCCGGAACAAGUGGUGGCGAUUGCAUCUAACGGAGGCGGG | |
| | AAGCAGGCGCUGGAGACAGUGCAACGGCUGCUCCCUGUUCUAUGC | |
| | CAAGCUCAUGGACUCACCCCCGAGCAAGUCGUCGCCAUUGCGUCC | |
| | AACAAUGGAGGCAAACAGGCACUGGAAACGGUGCAAAGACUGUUG | |
| | CCUGUACUGUGCCAGGCUCAUGGCCUUACUCCCGAACAGGUGGUG | |
| | GCAAUUGCGAGUAACAUCGGCGGAAAGCAGGCUUUGGAAACUGUG | |
| | CAGCGCUUGCUGCCUGUGCUCUGUCAGGCUCACGGACUCACCCCC | |
| | GAGCAAGUCGUAGCCAUCGCCUCAAAUAUCGGAGGCAAGCAAGCG | |
| | CUCGAGACUGUGCAGAGGCUCCUGCCUGUGUUAUGUCAGGCACAC | |
| | GGGCUGACCCCUGAACAAGUCGUGGCCAUUGCCUCCAACAACGGC | |
| | GGAAAACAGGCGCUCGAAACAGUCCAGAGGCUUUUGCCCGUGCUG | |
| | UGUCAAGCUCACGGCCUGACUCCGGAGCAAGUGGUCGCCAUUGCA | |
| | UCCCAUGAUGGCGGUAAACAAGCAUUGGAAACGGUCCAACGCCUG | |
| | CUGCCGGUCCUGUGCCAGGCCCAUGGUCUGACUCCCGAACAGGUC | |
| | GUGGCUAUCGCCUCCAACGGAGGUGGAAAACAAGCCCUGGAGACU | |
| | GUACAGAGGCUGCUGCCCGUGCUAUGUCAAGCACAUGGACUUACG | |
| | CCUGAACAAGUGGUGGCAAUUGCAUCACACGACGGAGGGAAGCAA | |
| | GCCCUUGAAACCGUGCAAAGGCUCCUCCCGGUGCUGUGCCAAGCC | |
| | CAUGGAUUGACUCCAGAACAGGUGGUCGCUAUCGCAUCACAUGAC | |
| | GGUGGAAAGCAAGCCCUCGAAACUGUGCAGCGGCUCCUCCCUGUG | |
| | UUGUGCCAGGCGCACGGUCUGACUCCCGAGCAAGUCGUGGCAAUC | |
| | GCCUCGAACAUCGGGGGCAAACAAGCUCUCGAAACUGUUCAAAGA | |
| | CUGCUCCCCGUGCUCUGUCAAGCUCACGGCCUCACUCCUGAACAG | |
| | GUAGUCGCGAUUGCCUCCCAUGACGGGGGAAAGCAGGCCUUGGAG | |
| | ACAGUCCAGCGGCUCUUGCCGGUCUUGUGCCAAGCACACGGAUUG | |
| | ACUCCAGAACAGGUGGUCGCAAUCGCCAGCAAUAUCGGGGGGAAG | |
| | CAAGCCUUGGAAACCGUUCAGCGGCUACUCCCUGUGCUGUGUCAA | |
| | GCCCACGGUCUGACCCCAGAACAAGUGGUGGCUAUCGCGUCGAAC | |
| | AUUGGAGGCAAACAGGCCCUCGAGACUGUCCAGAGACUCCUGCCA | |
| | GUCCUCUGCCAGGCUCAUGGGCUGACUCCUGAGCAAGUUGUGGCC | |
| | AUAGCAUCCAACGGCGGAGGAAAGCAAGCUCUGGAAACCGUACAG | |
| | AGACUUCUCCCGGUGUUGUGUCAGGCCCACGGACUUACUCCGGAA | |
| | CAGGUUGUCGCCAUCGCGUCCAACGGUGGUGGCAGGCCUGCUCUG | |
| | GAAUCCAUCGUGGCACAGUUGUCAAGACCAGAUCCUGCCCUGGCU | |
| | GCGCUCACCAACGACCACUUGGUGGCUCUGGCCUGCCUUGGCGGU | |
| | CGCCCUGCCUUGGAUGCCGUGAAGAAGGGACUUCCUCACGCCCCG | |
| | GCACUGAUUAAGCGCACCAAUCGCAGGAUCCCUGAACGGACCUCG | |
| | CACAGGGUC | |
| Effector protein | DAKSLTAWSRTLVTFKDVFVDFTREEWKLLDTAQQILYRNVMLEN | 13 |
| sequence 2 from | YKNLVSLGYQLTKPDVILRLEKGEEPWLVEREIHQETHPDSETAF | |
| bicistronic | EIKSSV | |
| construct | | |
| Effector RNA | GAUGCCAAGAGCUUGACCGCCUGGUCACGGACUCUUGUCACUUUC | 305 |
| sequence 2 from | AAGGAUGUCUUUGUGGACUUUACCAGAGAAGAAUGGAAGCUCCUG | |
| bicistronic | GACACUGCGCAGCAGAUCCUGUACAGAAACGUGAUGUUGGAAAAC | |
| construct | UACAAAAACCUGGUGUCACUCGGGUACCAACUCACCAAGCCCGAC | |
| | GUCAUCCUGCGCCUGGAAAAGGGCGAAGAACCGUGGUUGGUCGAG | |
| | AGAGAAAUCCAUCAGGAAACCCACCCUGACUCGGAAACCGCCUUC | |
| | GAGAUCAAGUCCUCGGUG | |
| First expression | MAPKKKRKVGIHGVPAAGSSGSLEPGEKPYKCPECGKSFSTSGNL | 306 |
| repressor, | VRHQRTHTGEKPYKCPECGKSFSRADNLTEHQRTHTGEKPYKCPE | |
| protein sequence | CGKSFSQSGDLRRHORTHTGEKPYKCPECGKSFSHKNALQNHQRT | |
| | HTGEKPYKCPECGKSFSDPGNLVRHORTHTGEKPYKCPECGKSFS | |
| | QRANLRAHQRTHTGEKPYKCPECGKSFSDCRDLARHQRTHTGKKT | |
| | SASGSGGGSGGDAKSLTAWSRTLVTFKDVFVDFTREEWKLLDTAQ | |
| | QILYRNVMLENYKNLVSLGYQLTKPDVILRLEKGEEPWLVEREIH | |
| | QETHPDSETAFEIKSSVSGGKRPAATKKAGQAKKKKGSGSATNFS | |
| | LLKQAGDVEENPG | |
| Second | PLEGSSGSGSPKKKRKVGIHGVPAAGSSGSHMVDLRTLGYSQQQQ | 307 |
| expression | EKIKPKVRSTVAQHHEALVGHGFTHAHIVALSQHPAALGTVAVKY | |
| repressor, | QDMIAALPEATHEAIVGVGKRGAGARALEALLTVAGELRGPPLQL | |
| protein sequence | DTGQLLKIAKRGGVTAVEAVHAWRNALTGAPLNLTPEQVVAIASN | |
| | GGGKQALETVQRLLPVLCQAHGLTPEQVVAIASNIGGKQALETVQ | |
| | RLLPVLCQAHGLTPEQVVAIASHDGGKQALETVQRLLPVLCQAHG | |
| | LTPEQVVAIASNGGGKQALETVORLLPVLCQAHGLTPEQVVAIAS | |
| | NNGGKQALETVQRLLPVLCQAHGLTPEQVVAIASNIGGKQALETV | |
| | QRLLPVLCQAHGLTPEQVVAIASNIGGKQALETVQRLLPVLCQAH | |
| | GLTPEQVVAIASNNGGKQALETVQRLLPVLCQAHGLTPEQVVAIA | |
| | SHDGGKQALETVQRLLPVLCQAHGLTPEQVVAIASNGGGKQALET | |
| | VQRLLPVLCQAHGLTPEQVVAIASHDGGKQALETVQRLLPVLCQA | |
| | HGLTPEQVVAIASHDGGKQALETVQRLLPVLCQAHGLTPEQVVAI | |
| | ASNIGGKQALETVQRLLPVLCQAHGLTPEQVVAIASHDGGKQALE | |
| | TVQRLLPVLCQAHGLTPEQVVAIASNIGGKQALETVQRLLPVLCQ | |
| | AHGLTPEQVVAIASNIGGKQALETVQRLLPVLCQAHGLTPEQVVA | |
| | IASNGGGKQALETVQRLLPVLCQAHGLTPEQVVAIASNGGGRPAL | |
| | ESIVAQLSRPDPALAALTNDHLVALACLGGRPALDAVKKGLPHAP | |
| | ALIKRTNRRIPERTSHRVASGSGGGSGGARDDAKSLTAWSRTLVT | |
| | FKDVFVDFTREEWKLLDTAQQILYRNVMLENYKNLVSLGYQLTKP | |
| | DVILRLEKGEEPWLVEREIHQETHPDSETAFEIKSSVPSSGGKRP | |
| | AATKKAGQAKKKKGSGS | |
| T2A peptide | PTSAGKLGSGEGRGSLLTCGDVEENPG | 340 |

In some embodiments, the bicistronic construct encodes a first expression repressor that binds the E1 locus at the target site GCCAAAGACATTGCACAGGAT (SEQ ID NO: 134). In some embodiments, the first expression repressor binds the E1 locus at chr4:74591896-74591916. In some embodiments, the bicistronic construct encodes a second expression repressor that binds the IL-S promoter at the target site TACTGAAGCTCCACAATT (SEQ ID NO: 292). In some embodiments, the second expression repressor binds the IL-8 promoter at GRCh37: chr4:74606039-74606056.

In some embodiments, the first expression repressor comprises a first targeting moiety having an amino acid sequence according to SEQ ID NO: 114, or a sequence having at least 80, 85, 90, 95, or 99% identity thereto. In some embodiments, first expression repressor comprises a first effector moiety having an amino acid sequence according to SEQ ID NO: 13, or a sequence having at least 80, 85, 90, 95, or 99% identity thereto, wherein optionally the first effector moiety is C-terminal of the first targeting moiety. In some embodiments, a linker is disposed between the first targeting moiety and the first effector moiety. In some embodiments, the first expression repressor comprises an NLS. In some embodiments, the first expression repressor has an amino acid sequence according to SEQ ID NO: 306, or a sequence having at least 80, 85, 90, 95, or 99% identity thereto.

In some embodiments, the second expression repressor comprises a second targeting moiety having an amino acid sequence according to SEQ ID NO: 268, or a sequence having at least 80, 85, 90, 95, or 99% identity thereto. In some embodiments, second expression repressor comprises a second effector moiety having an amino acid sequence according to SEQ ID NO: 13, or a sequence having at least 80, 85, 90, 95, or 99% identity thereto, wherein optionally the second effector moiety is C-terminal of the second targeting moiety. In some embodiments, a linker is disposed between the second targeting moiety and the second effector moiety. In some embodiments, the second expression repressor comprises an NLS. In some embodiments, the second expression repressor has an amino acid sequence according to SEQ ID NO: 307, or a sequence having at least 80, 85, 90, 95, or 99% identity thereto. In some embodiments, the second expression repressor is used together with the first expression repressor of the bicistronic construct. In other embodiments, the second expression repressor is used as a monotherapy or in combination with a second agent other than the first expression repressor.

In some embodiments, the first effector moiety and the second effector moiety have the same amino acid sequence. In other embodiments, the first effector moiety and the second effector moiety have different amino acid sequences.

In some embodiments, the bicistronic construct comprises a nucleic acid encoding the first repressor, wherein the first expression repressor comprises a first targeting moiety and a first effector moiety, wherein the nucleic acid encoding the first targeting moiety has a nucleic acid sequence according to SEQ ID NO: 302, or a sequence having at least 80, 85, 90, 95, or 99% identity thereto. In some embodiments, the nucleic acid encoding the first effector moiety has a nucleic acid sequence according to SEQ ID NO: 303, or a sequence having at least 80, 85, 90, 95, or 99% identity thereto.

In some embodiments, the bicistronic construct comprises a nucleic acid encoding the second expression repressor, wherein the second expression repressor comprises a targeting moiety and a second effector moiety, wherein the nucleic acid encoding the second targeting moiety has a nucleic acid sequence according to SEQ ID NO: 304, or a sequence having at least 80, 85, 90, 95, or 99% identity thereto. In some embodiments, the nucleic acid encoding the second effector moiety has a sequence according to SEQ ID NO: 305, or a sequence having at least 80, 85, 90, 95, or 99% identity thereto. In some embodiments, the nucleic acid encoding the second expression repressor is used together with a nucleic acid encoding the first expression repressor. In other embodiments, the nucleic acid encoding the second expression repressor is used as a monotherapy or in combination with a second agent other than a nucleic acid encoding the first expression repressor.

In some embodiments, the bicistronic construct comprises a nucleic acid having a sequence according to SEQ ID NO: 301, or a sequence having at least 80, 85, 90, 95, or 99% identity thereto. In some embodiments, the nucleic acid is RNA. In some embodiments, the nucleic acid is DNA.

In some embodiments, an expression repressor comprises a nuclear localization sequence (NLS). In some embodiments, the expression repressor comprises an NLS, e.g., an SV40 NLS at the N-terminus. In some embodiments, the expression repressor comprises an NLS, e.g., an SV40 NLS at the C-terminus. In some embodiments, the expression repressor comprises an NLS, e.g., a nucleoplasmin NLS at the C-terminus. In some embodiments, the expression repressor comprises a first NLS at the N-terminus and a second NLS at the C-terminus. In some embodiments the first and the second NLS have the same sequence. In some embodiments, the first and the second NLS have different sequences. In some embodiments, the expression repressor comprises a first NLS at the N-terminus, a second NLS, and a third NLS at the C-terminus. In some embodiments, at least two NLSs have the same sequence. In some embodiments, the first and the second NLS have the same sequence and the third NLS has a different sequence than the first and the second NLS. In some embodiments, the expression repressor comprises an SV40 NLS, e.g., the expression repressor comprises a sequence according to PKKKRK (SEQ ID NO: 63). In some embodiments, the site-specific disrupting agent comprises a nucleoplasmin NLS, e.g., the expression repressor comprises a sequence of KRPAATKKAGQAKKK (SEQ ID NO: 64). In some embodiments, the expression repressor comprises a C-terminal sequence comprising one or more of, e.g., any one or both of: a nucleoplasmin nuclear localization sequence and an HA-tag. In some embodiments, expression repressor comprises an epitope tag, e.g., an HA tag: YPYDVPDYA (SEQ ID NO: 65). In some embodiments, the expression repressor may comprise two copies of the epitope tag.

While an epitope tag is useful in many research contexts, it is sometimes desirable to omit an epitope tag in a therapeutic context. Accordingly, in some embodiments, the expression repressor lacks an epitope tag. In some embodiments, an expression repressor described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking the HA tag of SEQ ID NO: 65. In some embodiments, a nucleic acid described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking a region encoding the HA tag of SEQ ID NO: 65. In some embodiments, the expression repressor does not comprise an NLS. In some embodiments, the expression repressor does not comprise an epitope tag. In some embodiments the expression repressor does not comprise an HA tag. In some embodiments, the expression repressor does not comprise an HA tag sequence according to SEQ ID NO: 65.

In some embodiments, a nucleic acid for use in a method or composition described herein (e.g., a nucleic acid encoding an expression repressor) comprises a nucleic acid sequence of any one of SEQ ID NOs: 122-131, a complementary or reverse complementary sequence of any thereof, or comprises a sequence with at least 80, 85, 90, 91, 92, 93, 94, 95, 96, 97, 98, or 99% identity to any thereof. In some embodiments, an expression repressor for use in a method or composition described herein comprises an amino acid sequence of any one of SEQ ID NOs: 122-131, or comprises a sequence with at least 80, 85, 90, 91, 92, 93, 94, 95, 96, 97, 98, or 99% identity to any thereof. In some embodiments, an expression repressor for use in a method or composition described herein comprises an amino acid sequence encoded by any one of SEQ ID NOs: 122-131, or an amino acid sequence with at least 80, 85, 90, 91, 92, 93, 94, 95, 96, 97, 98, or 99% identity to any thereof.

In some embodiments, an expression repressor is encoded by a nucleic acid comprising a nucleic acid sequence of SEQ ID NO: 122, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 123, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 124, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 125, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 126, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 127, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 128, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 129, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 130, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 131, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

In some embodiments, an expression repressor is encoded by a nucleic acid sequence of SEQ ID NO: 142, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 143, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 144, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 145, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 146, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 147, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 148, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 149, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 150, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid sequence of SEQ ID NO: 151, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

In some embodiments, an expression repressor is encoded by a nucleic acid comprising a nucleic acid sequence of SEQ ID NO: 194, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 195, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 196, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 197, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 198, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 199, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

In some embodiments, an expression repressor is encoded by a nucleic acid comprising a nucleic acid sequence of SEQ ID NO: 248, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 249, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 250, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 251, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 252, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 253, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

In some embodiments, an expression repressor is encoded by a nucleic acid comprising a nucleic acid sequence of SEQ ID NO: 276, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 277, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 278, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 279, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 280, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 281, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 282, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 283, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

In some embodiments, an expression repressor is encoded by a nucleic acid comprising a nucleic acid sequence of SEQ ID NO: 284, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 285, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 286, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 287, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 288, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 289, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 290, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto. In some embodiments, an expression repressor is encoded by a nucleic acid comprising a nucleic acid sequence of SEQ ID NO: 291, or a nucleic acid sequence with at least 80%, 85%, 90%, 95%, 99%, or 100% identity thereto, or a nucleic acid sequence having no more than 20, 19, 18, 17, 16, 15, 14, 13, 12, 11, 10, 9, 8, 7, 6, 5, 4, 3, 2, or 1 positions of difference thereto.

In some embodiments, the present disclosure provides an expression repressor comprising a first targeting moiety (e.g., a targeting moiety comprising a first zinc finger targeting domain or a TAL targeting domain) and a first effector moiety for use in combination with a site-specific disrupting agent, for example a site-specific disrupting agent described herein.

### Exemplary Site-Specific Disrupting Agents

The following exemplary site-specific disrupting agents are presented for illustration purposes only and are not intended to be limiting.

In some embodiments, a site-specific disrupting agent comprises a targeting moiety (e.g., comprising dCas9, e.g., an S. aureus dCas9), and an effector moiety comprising MQ1, e.g., bacterial MQ1. In some embodiments, the site-specific disrupting agent is encoded by the nucleic acid sequence of SEQ ID NOs: 201 (e.g., a plasmid encoding the site-specific disrupting agent), and/or 202 (e.g., a nucleic acid (e.g., mRNA) encoding the site-specific disrupting agent), encoding the site-specific disrupting agent). In some embodiments, a nucleic acid described herein comprises a nucleic acid sequence of SEQ ID NO: 201 or 202 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the targeting moiety is encoded by the nucleic acid sequence of SEQ ID NO: 9 and/or the effector moiety is encoded by the nucleic acid sequence of SEQ ID NO: 10.

### Sa-dCas9-MQ1 (PL-27695) Plasmid DNA Sequence:

(SEQ ID NO: 201)
TCGCGCGTTTCGGTGATGACGGTGAAAACCTCTGACACATGCAGC
TCCCGGAGACGGTCACAGCTTGTCTGTAAGCGGATGCCGGGAGCA
GACAAGCCCGTCAGGGCGCGTCAGCGGGTGTTGGCGGGTGTCGGG
GCTGGCTTAACTATGCGGCATCAGAGCAGATTGTACTGAGAGTGC
ACCATATGCGGTGTGAAATACCGCACAGATGCGTAAGGAGAAAAT
ACCGCATCAGGCGCCATTCGCCATTCAGGCTGCGCAACTGTTGGG
AAGGGCGATCGGTGCGGGCCTCTTCGCTATTACGCCAGCTGGCGA
AAGGGGGATGTGCTGCAAGGCGATTAAGTTGGGTAACGCCAGGGT
TTTCCCAGTCACGACGTTGTAAAACGACGGCCAGTGAATTGACGC
GTATTGGGATGGTACCTAATACGACTCACTATAAGGAAATAAGAG
AGAAAAGAAGAGTAAGAAGAAATATAAGAGCCACCATGGCCCCCA
AGAAGAAGCGGAAGGTGGGCATCCACGGCGTGCCCGCCGCCGCCA
AGCGGAACTACATCCTGGGCCTGGCCATCGGCATCACCAGCGTGG
GCTACGGCATCATCGACTACGAGACCCGGGACGTGATCGACGCCG
GCGTGCGGCTGTTCAAGGAGGCCAACGTGGAGAACAACGAGGGCC
GGCGGAGCAAGCGGGGCGCCCGGCGGCTGAAGCGGCGGCGGCGGC
ACCGGATCCAGCGGGTGAAGAAGCTGCTGTTCGACTACAACCTGC
TGACCGACCACAGCGAGCTGAGCGGCATCAACCCCTACGAGGCCC
GGGTGAAGGGCCTGAGCCAGAAGCTGAGCGAGGAGGAGTTCAGCG
CCGCCCTGCTGCACCTGGCCAAGCGGCGGGGCGTGCACAACGTGA
ACGAGGTGGAGGAGGACACCGGCAACGAGCTGAGCACCAAGGAGC
AGATCAGCCGGAACAGCAAGGCCCTGGAGGAGAAGTACGTGGCCG
AGCTGCAGCTGGAGCGGCTGAAGAAGGACGGCGAGGTGCGGGGCA
GCATCAACCGGTTCAAGACCAGCGACTACGTGAAGGAGGCCAAGC
AGCTGCTGAAGGTGCAGAAGGCCTACCACCAGCTGGACCAGAGCT
TCATCGACACCTACATCGACCTGCTGGAGACCCGGCGGACCTACT
ACGAGGGCCCCGGCGAGGGCAGCCCCTTCGGCTGGAAGGACATCA
AGGAGTGGTACGAGATGCTGATGGGCCACTGCACCTACTTCCCCG
AGGAGCTGCGGAGCGTGAAGTACGCCTACAACGCCGACCTGTACA
ACGCCCTGAACGACCTGAACAACCTGGTGATCACCCGGGACGAGA
ACGAGAAGCTGGAGTACTACGAGAAGTTCCAGATCATCGAGAACG
TGTTCAAGCAGAAGAAGAAGCCCACCCTGAAGCAGATCGCCAAGG
AGATCCTGGTGAACGAGGAGGACATCAAGGGCTACCGGGTGACCA
GCACCGGCAAGCCCGAGTTCACCAACCTGAAGGTGTACCACGACA
TCAAGGACATCACCGCCCGGAAGGAGATCATCGAGAACGCCGAGC
TGCTGGACCAGATCGCCAAGATCCTGACCATCTACCAGAGCAGCG
AGGACATCCAGGAGGAGCTGACCAACCTGAACAGCGAGCTGACCC
AGGAGGAGATCGAGCAGATCAGCAACCTGAAGGGCTACACCGGCA
CCCACAACCTGAGCCTGAAGGCCATCAACCTGATCCTGGACGAGC
TGTGGCACACCAACGACAACCAGATCGCCATCTTCAACCGGCTGA
AGCTGGTGCCCAAGAAGGTGGACCTGAGCCAGCAGAAGGAGATCC
CCACCACCCTGGTGGACGACTTCATCCTGAGCCCCGTGGTGAAGC
GGAGCTTCATCCAGAGCATCAAGGTGATCAACGCCATCATCAAGA
AGTACGGCCTGCCCAACGACATCATCATCGAGCTGGCCCGGGAGA
AGAACAGCAAGGACGCCCAGAAGATGATCAACGAGATGCAGAAGC
GGAACCGGCAGACCAACGAGCGGATCGAGGAGATCATCCGGACCA
CCGGCAAGGAGAACGCCAAGTACCTGATCGAGAAGATCAAGCTGC
ACGACATGCAGGAGGGCAAGTGCCTGTACAGCCTGGAGGCCATCC
CCCTGGAGGACCTGCTGAACAACCCCTTCAACTACGAGGTGGACG
CCATCATCCCCCGGAGCGTGAGCTTCGACAACAGCTTCAACAACA
AGGTGCTGGTGAAGCAGGAGGAGAACAGCAAGAAGGGCAACCGGA
CCCCCTTCCAGTACCTGAGCAGCAGCGACAGCAAGATCAGCTACG
AGACCTTCAAGAAGCACATCCTGAACCTGGCCAAGGGCAAGGGCC
GGATCAGCAAGACCAAGAAGGAGTACCTGCTGGAGGAGCGGGACA
TCAACCGGTTCAGCGTGCAGAAGGACTTCATCAACCGGAACCTGG
TGGACACCCGGTACGCCACCCGGGGCCTGATGAACCTGCTGCGGA
GCTACTTCCGGGTGAACAACCTGGACGTGAAGGTGaaatccATCA
ACGGCGGCTTCACCAGCTTCCTGCGGCGGAAGTGGAAGTTCAAGA
AGGAGCGGAACAAGGGCTACAAGCACCACGCCGAGGACGCCCTGA
TCATCGCCAACGCCGACTTCATCTTCAAGGAGTGGAAGAAGCTGG
ACAAGGCCAAGAAGGTGATGGAGAACCAGATGTTCGAGGAGAAGC
AGGCCGAGAGCATGCCCGAGATCGAGACCGAGCAGGAGTACAAGG
AGATCTTCATCACCCCCCACCAGATCAAGCACATCAAGGACTTCA
AGGACTACAAGTACAGCCACCGGGTGGACAAGAAGCCCAACCGGA
AGCTGATCAACGACACCCTGTACAGCACCCGGAAGGACGACAAGG
GCAACACCCTGATCGTGAACAACCTGAACGGCCTGTACGACAAGG
ACAACGACAAGCTGAAGAAGCTGATCAACAAGAGCCCCGAGAAGC
TGCTGATGTACCACCACGACCCCCAGACCTACCAGAAGCTGAAGC
TGATCATGGAGCAGTACGGCGACGAGAAGAACCCCCTGTACAAGT
ACTACGAGGAGACCGGCAACTACCTGACCAAGTACAGCAAGAAGG
ACAACGGCCCCGTGATCAAGAAGATCAAGTACTACGGCAACAAGC
TGAACGCCCACCTGGACATCACCGACGACTACCCCAACAGCCGGA
ACAAGGTGGTGAAGCTGAGCCTGAAGCCCTACCGGTTCGACGTGT
ACCTGGACAACGGCGTGTACAAGTTCGTGACCGTGAAGAACCTGG
ACGTGATCAAGAAGGAGAACTACTACGAGGTGAACAGCAAGTGCT
ACGAGGAGGCCAAGAAGCTGAAGAAGATCAGCAACCAGGCCGAGT
TCATCGCCAGCTTCTACAAGAACGACCTGATCAAGATCAACGGCG
AGCTGTACCGGGTGATCGGCGTGAACAACGACCTGCTGAACCGGA
TCGAGGTGAACATGATCGACATCACCTACCGGGAGTACCTGGAGA
ACATGAACGACAAGCGGCCCCCCCACATCATCAAGACCATCGCCA
GCAAGACCCAGAGCATCAAGAAGTACAGCACCGACATCCTGGGCA
ACCTGTACGAGGTGaaatccAAGAAGCACCCCCAGATCATCAAGA
AGGGCAAGCGGCCCGCCGCCACCAAGAAGGCCGGCCAGGCCAAGA
AGAAGAAGGCCCGGGACAGCAAGGTGGAGAACAAGACCAAGAAGC
TGCGGGTGTTCGAGGCCTTCGCCGGCATCGGCGCCCAGCGGAAGG
CCCTGGAGAAGGTGCGGAAGGACGAGTACGAGATCGTGGGCCTGG
CCGAGTGGTACGTGCCCGCCATCGTGATGTACCAGGCCATCCACA
ACAACTTCCACACCAAGCTGGAGTACAAGAGCGTGAGCCGGGAGG
AGATGATCGACTACCTGGAGAACAAGACCCTGAGCTGGAACAGCA
AGAACCCCGTGAGCAACGGCTACTGGAAGCGGAAGAAGGACGACG
AGCTGAAGATCATCTACAACGCCATCAAGCTGAGCGAGAAGGAGG
GCAACATCTTCGACATCCGGGACCTGTACAAGCGGACCCTGAAGA
ACATCGACCTGCTGACCTACAGCTTCCCCTGCCAGGACCTGAGCC
AGCAGGGCATCCAGAAGGGCATGAAGCGGGGCAGCGGCACCCGGA
GCGGCCTGCTGTGGGAGATCGAGCGGGCCCTGGACAGCACCGAGA
AGAACGACCTGCCCAAGTACCTGCTGATGGAGAACGTGGGCGCCC
TGCTGCACAAGAAGAACGAGGAGGAGCTGAACCAGTGGAAGCAGA
AGCTGGAGAGCCTGGGCTACCAGAACAGCATCGAGGTGCTGAACG
CCGCCGACTTCGGCAGCAGCCAGGCCCGGCGGCGGGTGTTCATGA
TCAGCACCCTGAACGAGTTCGTGGAGCTGCCCAAGGGCGACAAGA
AGCCCAAGAGCATCAAGAAGGTGCTGAACAAGATCGTGAGCGAGA
AGGACATCCTGAACAACCTGCTGAAGTACAACCTGACCGAGTTCA
AGAAaACCAAGAGCAACATCAACAAGGCCAGCCTGATCGGCTACA
GCAAGTTCAACAGCGAGGGCTACGTGTACGACCCCGAGTTCACCG
GCCCCACCCTGACCGCCAGCGGCGCCAACAGCCGGATCAAGATCA
AGGACGGCAGCAACATCCGGAAGATGAACAGCGACGAGACCTTCC
TGTACATCGGCTTCGACAGCCAGGACGGCAAGCGGGTGAACGAGA
TCGAGTTCCTGACCGAGAACCAGAAGATCTTCGTGTGCGGCAACA
GCATCAGCGTGGAGGTGCTGGAGGCCATCATCGACAAGATCGGCG
GCCCCAGCAGCGGCGGCAAGCGGCCCGCCGCCACCAAGAAGGCCG
GCCAGGCCAAGAAGAAGAAGGGCAGCTACCCCTACGACGTGCCCG
ACTACGCCTGAGcggccgcttaattaagctgccttctgcggggct
tgccttctggccatgcccttcttctctcccttgcacctgtacctc
ttggtctttgaaaaaaaaattgtcttcttcatcgcctgcagATCC
CAATGGCGCGCCGAGCTTGGCTCGAGCATGGTCATAGCTGTTTCC
TGTGTGAAATTGTTATCCGCTCACAATTCCACACAACATACGAGC
CGGAAGCATAAAGTGTAAAGCCTGGGGTGCCTAATGAGTGAGCTA
ACTCACATTAATTGCGTTGCGCTCACTGCCCGCTTTCCAGTCGGG
AAACCTGTCGTGCCAGCTGCATTAATGAATCGGCCAACGCGCGGG
GAGAGGCGGTTTGCGTATTGGGCGCTCTTCCGCTTCCTCGCTCAC
TGACTCGCTGCGCTCGGTCGTTCGGCTGCGGCGAGCGGTATCAGC
TCACTCAAAGGCGGTAATACGGTTATCCACAGAATCAGGGGATAA
CGCAGGAAAGAACATGTGAGCAAAAGGCCAGCAAAAGGCCAGGAA
CCGTAAAAAGGCCGCGTTGCTGGCGTTTTTCCATAGGCTCCGCCC
CCCTGACGAGCATCACAAAAATCGACGCTCAAGTCAGAGGTGGCG
AAACCCGACAGGACTATAAAGATACCAGGCGTTTCCCCCTGGAAG
CTCCCTCGTGCGCTCTCCTGTTCCGACCCTGCCGCTTACCGGATA
CCTGTCCGCCTTTCTCCCTTCGGGAAGCGTGGCGCTTTCTCATAG
CTCACGCTGTAGGTATCTCAGTTCGGTGTAGGTCGTTCGCTCCAA
GCTGGGCTGTGTGCACGAACCCCCCGTTCAGCCCGACCGCTGCGC
CTTATCCGGTAACTATCGTCTTGAGTCCAACCCGGTAAGACACGA
CTTATCGCCACTGGCAGCAGCCACTGGTAACAGGATTAGCAGAGC
GAGGTATGTAGGCGGTGCTACAGAGTTCTTGAAGTGGTGGCCTAA
CTACGGCTACACTAGAAGAACAGTATTTGGTATCTGCGCTCTGCT
GAAGCCAGTTACCTTCGGAAAAAGAGTTGGTAGCTCTTGATCCGG
CAAACAAACCACCGCTGGTAGCGGTGGTTTTTTTGTTTGCAAGCA
GCAGATTACGCGCAGAAAAAAAGGATCTCAAGAAGATCCTTTGAT
CTTTTCTACGGGGTCTGACGCTCAGTGGAACGAAAACTCACGTTA
AGGGATTTTGGTCATGAGATTATCAAAAAGGATCTTCACCTAGAT
CCTTTTAAATTAAAAATGAAGTTTTAAATCAATCTAAAGTATATA
TGAGTAAACTTGGTCTGACAGTTAGAAAAACTCATCGAGCATCAA
ATGAAACTGCAATTTATTCATATCAGGATTATCAATACCATATTT
TTGAAAAAGCCGTTTCTGTAATGAAGGAGAAAACTCACCGAGGCA
GTTCCATAGGATGGCAAGATCCTGGTATCGGTCTGCGATTCCGAC
TCGTCCAACATCAATACAACCTATTAATTTCCCCTCGTCAAAAAT
AAGGTTATCAAGTGAGAAATCACCATGAGTGACGACTGAATCCGG
TGAGAATGGCAAAAGTTTATGCATTTCTTTCCAGACTTGTTCAAC
AGGCCAGCCATTACGCTCGTCATCAAAATCACTCGCATCAACCAA
ACCGTTATTCATTCGTGATTGCGCCTGAGCGAGACGAAATACGCG
ATCGCTGTTAAAAGGACAATTACAAACAGGAATCGAATGCAACCG
GCGCAGGAACACTGCCAGCGCATCAACAATATTTTCACCTGAATC
AGGATATTCTTCTAATACCTGGAATGCTGTTTTCCCAGGGATCGC
AGTGGTGAGTAACCATGCATCATCAGGAGTACGGATAAAATGCTT
GATGGTCGGAAGAGGCATAAATTCCGTCAGCCAGTTTAGTCTGAC
CATCTCATCTGTAACATCATTGGCAACGCTACCTTTGCCATGTTT
CAGAAACAACTCTGGCGCATCGGGCTTCCCATACAATCGATAGAT
TGTCGCACCTGATTGCCCGACATTATCGCGAGCCCATTTATACCC
ATATAAATCAGCATCCATGTTGGAATTTAATCGCGGCCTAGAGCA
AGACGTTTCCCGTTGAATATGGCTCATACTCTTCCTTTTTCAATA
TTATTGAAGCATTTATCAGGGTTATTGTCTCATGAGCGGATACAT
ATTTGAATGTATTTAGAAAAATAAACAAATAGGGGTTCCGCGCAC
ATTTCCCCGAAAAGTGCCACCTGACGTCTAAGAAACCATTATTAT
CATGACATTAACCTATAAAAATAGGCGTATCACGAGGCCCTTTCG
TC

Sa-dCas9-MQ1 (MR-28126) Expressed mRNA Sequence:

(SEQ ID NO: 202)
AGGAAATAAGAGAGAAAAGAAGAGTAAGAAGAAATATAAGAGCCA
CCATGGCCCCCAAGAAGAAGCGGAAGGTGGGCATCCACGGCGTGC
CCGCCGCCGCCAAGCGGAACTACATCCTGGGCCTGGCCATCGGCA
TCACCAGCGTGGGCTACGGCATCATCGACTACGAGACCCGGGACG
TGATCGACGCCGGCGTGCGGCTGTTCAAGGAGGCCAACGTGGAGA
ACAACGAGGGCCGGCGGAGCAAGCGGGGCGCCCGGCGGCTGAAGC
GGCGGCGGCGGCACCGGATCCAGCGGGTGAAGAAGCTGCTGTTCG
ACTACAACCTGCTGACCGACCACAGCGAGCTGAGCGGCATCAACC
CCTACGAGGCCCGGGTGAAGGGCCTGAGCCAGAAGCTGAGCGAGG
AGGAGTTCAGCGCCGCCCTGCTGCACCTGGCCAAGCGGCGGGGCG
TGCACAACGTGAACGAGGTGGAGGAGGACACCGGCAACGAGCTGA
GCACCAAGGAGCAGATCAGCCGGAACAGCAAGGCCCTGGAGGAGA
AGTACGTGGCCGAGCTGCAGCTGGAGCGGCTGAAGAAGGACGGCG
AGGTGCGGGGCAGCATCAACCGGTTCAAGACCAGCGACTACGTGA
AGGAGGCCAAGCAGCTGCTGAAGGTGCAGAAGGCCTACCACCAGC
TGGACCAGAGCTTCATCGACACCTACATCGACCTGCTGGAGACCC
GGCGGACCTACTACGAGGGCCCCGGCGAGGGCAGCCCCTTCGGCT
GGAAGGACATCAAGGAGTGGTACGAGATGCTGATGGGCCACTGCA
CCTACTTCCCCGAGGAGCTGCGGAGCGTGAAGTACGCCTACAACG
CCGACCTGTACAACGCCCTGAACGACCTGAACAACCTGGTGATCA
CCCGGGACGAGAACGAGAAGCTGGAGTACTACGAGAAGTTCCAGA
TCATCGAGAACGTGTTCAAGCAGAAGAAGAAGCCCACCCTGAAGC
AGATCGCCAAGGAGATCCTGGTGAACGAGGAGGACATCAAGGGCT
ACCGGGTGACCAGCACCGGCAAGCCCGAGTTCACCAACCTGAAGG
TGTACCACGACATCAAGGACATCACCGCCCGGAAGGAGATCATCG
AGAACGCCGAGCTGCTGGACCAGATCGCCAAGATCCTGACCATCT
ACCAGAGCAGCGAGGACATCCAGGAGGAGCTGACCAACCTGAACA
GCGAGCTGACCCAGGAGGAGATCGAGCAGATCAGCAACCTGAAGG
GCTACACCGGCACCCACAACCTGAGCCTGAAGGCCATCAACCTGA
TCCTGGACGAGCTGTGGCACACCAACGACAACCAGATCGCCATCT
TCAACCGGCTGAAGCTGGTGCCCAAGAAGGTGGACCTGAGCCAGC
AGAAGGAGATCCCCACCACCCTGGTGGACGACTTCATCCTGAGCC
CCGTGGTGAAGCGGAGCTTCATCCAGAGCATCAAGGTGATCAACG
CCATCATCAAGAAGTACGGCCTGCCCAACGACATCATCATCGAGC
TGGCCCGGGAGAAGAACAGCAAGGACGCCCAGAAGATGATCAACG
AGATGCAGAAGCGGAACCGGCAGACCAACGAGCGGATCGAGGAGA
TCATCCGGACCACCGGCAAGGAGAACGCCAAGTACCTGATCGAGA
AGATCAAGCTGCACGACATGCAGGAGGGCAAGTGCCTGTACAGCC
TGGAGGCCATCCCCCTGGAGGACCTGCTGAACAACCCCTTCAACT
ACGAGGTGGACGCCATCATCCCCCGGAGCGTGAGCTTCGACAACA
GCTTCAACAACAAGGTGCTGGTGAAGCAGGAGGAGAACAGCAAGA
AGGGCAACCGGACCCCCTTCCAGTACCTGAGCAGCAGCGACAGCA
AGATCAGCTACGAGACCTTCAAGAAGCACATCCTGAACCTGGCCA
AGGGCAAGGGCCGGATCAGCAAGACCAAGAAGGAGTACCTGCTGG
AGGAGCGGGACATCAACCGGTTCAGCGTGCAGAAGGACTTCATCA
ACCGGAACCTGGTGGACACCCGGTACGCCACCCGGGGCCTGATGA
ACCTGCTGCGGAGCTACTTCCGGGTGAACAACCTGGACGTGAAGG
TGaaatccATCAACGGCGGCTTCACCAGCTTCCTGCGGCGGAAGT
GGAAGTTCAAGAAGGAGCGGAACAAGGGCTACAAGCACCACGCCG
AGGACGCCCTGATCATCGCCAACGCCGACTTCATCTTCAAGGAGT
GGAAGAAGCTGGACAAGGCCAAGAAGGTGATGGAGAACCAGATGT
TCGAGGAGAAGCAGGCCGAGAGCATGCCCGAGATCGAGACCGAGC
AGGAGTACAAGGAGATCTTCATCACCCCCCACCAGATCAAGCACA
TCAAGGACTTCAAGGACTACAAGTACAGCCACCGGGTGGACAAGA
AGCCCAACCGGAAGCTGATCAACGACACCCTGTACAGCACCCGGA
AGGACGACAAGGGCAACACCCTGATCGTGAACAACCTGAACGGCC
TGTACGACAAGGACAACGACAAGCTGAAGAAGCTGATCAACAAGA
GCCCCGAGAAGCTGCTGATGTACCACCACGACCCCCAGACCTACC
AGAAGCTGAAGCTGATCATGGAGCAGTACGGCGACGAGAAGAACC
CCCTGTACAAGTACTACGAGGAGACCGGCAACTACCTGACCAAGT
ACAGCAAGAAGGACAACGGCCCCGTGATCAAGAAGATCAAGTACT
ACGGCAACAAGCTGAACGCCCACCTGGACATCACCGACGACTACC
CCAACAGCCGGAACAAGGTGGTGAAGCTGAGCCTGAAGCCCTACC
GGTTCGACGTGTACCTGGACAACGGCGTGTACAAGTTCGTGACCG
TGAAGAACCTGGACGTGATCAAGAAGGAGAACTACTACGAGGTGA
ACAGCAAGTGCTACGAGGAGGCCAAGAAGCTGAAGAAGATCAGCA
ACCAGGCCGAGTTCATCGCCAGCTTCTACAAGAACGACCTGATCA
AGATCAACGGCGAGCTGTACCGGGTGATCGGCGTGAACAACGACC
TGCTGAACCGGATCGAGGTGAACATGATCGACATCACCTACCGGG
AGTACCTGGAGAACATGAACGACAAGCGGCCCCCCCACATCATCA
AGACCATCGCCAGCAAGACCCAGAGCATCAAGAAGTACAGCACCG
ACATCCTGGGCAACCTGTACGAGGTGaaatccAAGAAGCACCCCC
AGATCATCAAGAAGGGCAAGCGGCCCGCCGCCACCAAGAAGGCCG
GCCAGGCCAAGAAGAAGAAGGCCCGGGACAGCAAGGTGGAGAACA
AGACCAAGAAGCTGCGGGTGTTCGAGGCCTTCGCCGGCATCGGCG
CCCAGCGGAAGGCCCTGGAGAAGGTGCGGAAGGACGAGTACGAGA
TCGTGGGCCTGGCCGAGTGGTACGTGCCCGCCATCGTGATGTACC
AGGCCATCCACAACAACTTCCACACCAAGCTGGAGTACAAGAGCG
TGAGCCGGGAGGAGATGATCGACTACCTGGAGAACAAGACCCTGA
GCTGGAACAGCAAGAACCCCGTGAGCAACGGCTACTGGAAGCGGA
AGAAGGACGACGAGCTGAAGATCATCTACAACGCCATCAAGCTGA
GCGAGAAGGAGGGCAACATCTTCGACATCCGGGACCTGTACAAGC
GGACCCTGAAGAACATCGACCTGCTGACCTACAGCTTCCCCTGCC
AGGACCTGAGCCAGCAGGGCATCCAGAAGGGCATGAAGCGGGGCA
GCGGCACCCGGAGCGGCCTGCTGTGGGAGATCGAGCGGGCCCTGG
ACAGCACCGAGAAGAACGACCTGCCCAAGTACCTGCTGATGGAGA
ACGTGGGCGCCCTGCTGCACAAGAAGAACGAGGAGGAGCTGAACC
AGTGGAAGCAGAAGCTGGAGAGCCTGGGCTACCAGAACAGCATCG
AGGTGCTGAACGCCGCCGACTTCGGCAGCAGCCAGGCCCGGCGGC
GGGTGTTCATGATCAGCACCCTGAACGAGTTCGTGGAGCTGCCCA
AGGGCGACAAGAAGCCCAAGAGCATCAAGAAGGTGCTGAACAAGA
TCGTGAGCGAGAAGGACATCCTGAACAACCTGCTGAAGTACAACC
TGACCGAGTTCAAGAAaACCAAGAGCAACATCAACAAGGCCAGCC
TGATCGGCTACAGCAAGTTCAACAGCGAGGGCTACGTGTACGACC
CCGAGTTCACCGGCCCCACCCTGACCGCCAGCGGCGCCAACAGCC
GGATCAAGATCAAGGACGGCAGCAACATCCGGAAGATGAACAGCG
ACGAGACCTTCCTGTACATCGGCTTCGACAGCCAGGACGGCAAGC
GGGTGAACGAGATCGAGTTCCTGACCGAGAACCAGAAGATCTTCG
TGTGCGGCAACAGCATCAGCGTGGAGGTGCTGGAGGCCATCATCG
ACAAGATCGGCGGCCCCAGCAGCGGCGGCAAGCGGCCCGCCGCCA
CCAAGAAGGCCGGCCAGGCCAAGAAGAAGAAGGGCAGCTACCCCT
ACGACGTGCCCGACTACGCCTGAGcggccgcttaattaagctgcc
ttctgcggggcttgccttctggccatgcccttcttctctcccttg
cacctgtacctcttggtctttgaataaagcctgagtaggaagtct
agaaaaaaaaaaaaaaaaaaaaa

In some embodiments, a site-specific disrupting agent comprises a targeting moiety (e.g., comprising dCas9, e.g., an S. pyogenes dCas9 or a functional variant or mutant thereof; e.g., Cas9m4), and an effector moiety comprising MQ1, e.g., bacterial MQ1. In some embodiments, the site-specific disrupting agent is encoded by the nucleic acid sequence of SEQ ID NOs: 207 (e.g., a nucleic acid (e.g., mRNA) encoding the site-specific disrupting agent). In some embodiments, a nucleic acid described herein comprises a nucleic acid sequence of SEQ ID NO: 207 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the targeting moiety is encoded by the nucleic acid sequence of SEQ ID NO: 8 and/or the effector moiety is encoded by the nucleic acid sequence of SEQ ID NO: 10.

dCas9-MQ1 mRNA Sequence (MR28125)

(SEQ ID NO: 207)
AAGGAAAUAAGAGAGAAAAGAAGAGUAAGAAGAAAUAUAAGAGCC
ACCAUGGCCCCCAAGAAGAAGCGGAAGGUGGGCAUCCACGGCGUG
CCCGCCGCCGACAAGAAGUACAGCAUCGGCCUGGCCAUCGGCACC
AACAGCGUGGGCUGGGCCGUGAUCACCGACGAGUACAAGGUGCCC
AGCAAGAAGUUCAAGGUGCUGGGCAACACCGACCGGCACAGCAUC
AAGAAGAACCUGAUCGGCGCCCUGCUGUUCGACAGCGGCGAGACC
GCCGAGGCCACCCGGCUGAAGCGGACCGCCCGGCGGCGGUACACC
CGGCGGAAGAACCGGAUCUGCUACCUGCAGGAGAUCUUCAGCAAC
GAGAUGGCCAAGGUGGACGACAGCUUCUUCCACCGGCUGGAGGAG
AGCUUCCUGGUGGAGGAGGACAAGAAGCACGAGCGGCACCCCAUC
UUCGGCAACAUCGUGGACGAGGUGGCCUACCACGAGAAGUACCCC
ACCAUCUACCACCUGCGGAAGAAGCUGGUGGACAGCACCGACAAG
GCCGACCUGCGGCUGAUCUACCUGGCCCUGGCCCACAUGAUCAAG
UUCCGGGGCCACUUCCUGAUCGAGGGCGACCUGAACCCCGACAAC
AGCGACGUGGACAAGCUGUUCAUCCAGCUGGUGCAGACCUACAAC
CAGCUGUUCGAGGAGAACCCCAUCAACGCCAGCGGCGUGGACGCC
AAGGCCAUCCUGAGCGCCCGGCUGAGCAAGAGCCGGCGGCUGGAG
AACCUGAUCGCCCAGCUGCCCGGCGAGAAGAAGAACGGCCUGUUC
GGCAACCUGAUCGCCCUGAGCCUGGGCCUGACCCCCAACUUCAAG
AGCAACUUCGACCUGGCCGAGGACGCCAAGCUGCAGCUGAGCAAG
GACACCUACGACGACGACCUGGACAACCUGCUGGCCCAGAUCGGC
GACCAGUACGCCGACCUGUUCCUGGCCGCCAAGAACCUGAGCGAC
GCCAUCCUGCUGAGCGACAUCCUGCGGGUGAACACCGAGAUCACC
AAGGCCCCCCUGAGCGCCAGCAUGAUCAAGCGGUACGACGAGCAC
CACCAGGACCUGACCCUGCUGAAGGCCCUGGUGCGGCAGCAGCUG
CCCGAGAAGUACAAGGAGAUCUUCUUCGACCAGAGCAAGAACGGC
UACGCCGGCUACAUCGACGGCGGCGCCAGCCAGGAGGAGUUCUAC
AAGUUCAUCAAGCCCAUCCUGGAGAAGAUGGACGGCACCGAGGAG
CUGCUGGUGAAGCUGAACCGGGAGGACCUGCUGCGGAAGCAGCGG
ACCUUCGACAACGGCAGCAUCCCCCACCAGAUCCACCUGGGCGAG
CUGCACGCCAUCCUGCGGCGGCAGGAGGACUUCUACCCCUUCCUG
AAGGACAACCGGGAGAAGAUCGAGAAGAUCCUGACCUUCCGGAUC
CCCUACUACGUGGGCCCCCUGGCCCGGGGCAACAGCCGGUUCGCC
UGGAUGACCCGGAAAUCCGAGGAGACCAUCACCCCCUGGAACUUC
GAGGAGGUGGUGGACAAGGGCGCCAGCGCCCAGAGCUUCAUCGAG
CGGAUGACCAACUUCGACAAGAACCUGCCCAACGAGAAGGUGCUG
CCCAAGCACAGCCUGCUGUACGAGUACUUCACCGUGUACAACGAG
CUGACCAAGGUGAAGUACGUGACCGAGGGCAUGCGGAAGCCCGCC
UUCCUGAGCGGCGAGCAGAAGAAGGCCAUCGUGGACCUGCUGUUC
AAGACCAACCGGAAGGUGACCGUGAAGCAGCUGAAGGAGGACUAC
UUCAAGAAGAUCGAGUGCUUCGACAGCGUGGAGAUCAGCGGCGUG
GAGGACCGGUUCAACGCCAGCCUGGGCACCUACCACGACCUGCUG
AAGAUCAUCAAGGACAAGGACUUCCUGGACAACGAGGAGAACGAG
GACAUCCUGGAGGACAUCGUGCUGACCCUGACCCUGUUCGAGGAC
CGGGAGAUGAUCGAGGAGCGGCUGAAAACCUACGCCCACCUGUUC
GACGACAAGGUGAUGAAGCAGCUGAAGCGGCGGCGGUACACCGGC
UGGGGCCGGCUGAGCCGGAAGCUGAUCAACGGCAUCCGGGACAAG
CAGAGCGGCAAGACCAUCCUGGACUUCCUGAAAUCCGACGGCUUC
GCCAACCGGAACUUCAUGCAGCUGAUCCACGACGACAGCCUGACC
UUCAAGGAGGACAUCCAGAAGGCCCAGGUGAGCGGCCAGGGCGAC
AGCCUGCACGAGCACAUCGCCAACCUGGCCGGCAGCCCCGCCAUC
AAGAAGGGCAUCCUGCAGACCGUGAAGGUGGUGGACGAGCUGGUG
AAGGUGAUGGGCCGGCACAAGCCCGAGAACAUCGUGAUCGAGAUG
GCCCGGGAGAACCAGACCACCCAGAAGGGCCAGAAGAACAGCCGG
GAGCGGAUGAAGCGGAUCGAGGAGGGCAUCAAGGAGCUGGGCAGC
CAGAUCCUGAAGGAGCACCCCGUGGAGAACACCCAGCUGCAGAAC
GAGAAGCUGUACCUGUACUACCUGCAGAACGGCCGGGACAUGUAC
GUGGACCAGGAGCUGGACAUCAACCGGCUGAGCGACUACGACGUG
GCCGCCAUCGUGCCCCAGAGCUUCCUGAAGGACGACAGCAUCGAC
AACAAGGUGCUGACCCGGAGCGACAAGGCCCGGGGCAAGAGCGAC
AACGUGCCCAGCGAGGAGGUGGUGAAGAAGAUGAAGAACUACUGG
CGGCAGCUGCUGAACGCCAAGCUGAUCACCCAGCGGAAGUUCGAC
AACCUGACCAAGGCCGAGCGGGGCGGCCUGAGCGAGCUGGACAAG
GCCGGCUUCAUCAAGCGGCAGCUGGUGGAGACCCGGCAGAUCACC
AAGCACGUGGCCCAGAUCCUGGACAGCCGGAUGAACACCAAGUAC
GACGAGAACGACAAGCUGAUCCGGGAGGUGAAGGUGAUCACCCUG
AAAUCCAAGCUGGUGAGCGACUUCCGGAAGGACUUCCAGUUCUAC
AAGGUGCGGGAGAUCAACAACUACCACCACGCCCACGACGCCUAC
CUGAACGCCGUGGUGGGCACCGCCCUGAUCAAGAAGUACCCCAAG
CUGGAGAGCGAGUUCGUGUACGGCGACUACAAGGUGUACGACGUG
CGGAAGAUGAUCGCCAAGAGCGAGCAGGAGAUCGGCAAGGCCACC
GCCAAGUACUUCUUCUACAGCAACAUCAUGAACUUCUUCAAGACC
GAGAUCACCCUGGCCAACGGCGAGAUCCGGAAGCGGCCCCUGAUC
GAGACCAACGGCGAGACCGGCGAGAUCGUGUGGGACAAGGGCCGG
GACUUCGCCACCGUGCGGAAGGUGCUGAGCAUGCCCCAGGUGAAC
AUCGUGAAGAAAACCGAGGUGCAGACCGGCGGCUUCAGCAAGGAG
AGCAUCCUGCCCAAGCGGAACAGCGACAAGCUGAUCGCCCGGAAG
AAGGACUGGGACCCCAAGAAGUACGGCGGCUUCGACAGCCCCACC
GUGGCCUACAGCGUGCUGGUGGUGGCCAAGGUGGAGAAGGGCAAG
AGCAAGAAGCUGAAAUCCGUGAAGGAGCUGCUGGGCAUCACCAUC
AUGGAGCGGAGCAGCUUCGAGAAGAACCCCAUCGACUUCCUGGAG
GCCAAGGGCUACAAGGAGGUGAAGAAGGACCUGAUCAUCAAGCUG
CCCAAGUACAGCCUGUUCGAGCUGGAGAACGGCCGGAAGCGGAUG
CUGGCCAGCGCCGGCGAGCUGCAGAAGGGCAACGAGCUGGCCCUG
CCCAGCAAGUACGUGAACUUCCUGUACCUGGCCAGCCACUACGAG
AAGCUGAAGGGCAGCCCCGAGGACAACGAGCAGAAGCAGCUGUUC
GUGGAGCAGCACAAGCACUACCUGGACGAGAUCAUCGAGCAGAUC
AGCGAGUUCAGCAAGCGGGUGAUCCUGGCCGACGCCAACCUGGAC
AAGGUGCUGAGCGCCUACAACAAGCACCGGGACAAGCCCAUCCGG
GAGCAGGCCGAGAACAUCAUCCACCUGUUCACCCUGACCAACCUG
GGCGCCCCCGCCGCCUUCAAGUACUUCGACACCACCAUCGACCGG
AAGCGGUACACCAGCACCAAGGAGGUGCUGGACGCCACCCUGAUC
CACCAGAGCAUCACCGGCCUGUACGAGACCCGGAUCGACCUGAGC
CAGCUGGGCGGCGACAAGCGGCCCGCCGCCACCAAGAAGGCCGGC
CAGGCCAAGAAGAAGAAGGCCCGGGACAGCAAGGUGGAGAACAAG
ACCAAGAAGCUGCGGGUGUUCGAGGCCUUCGCCGGCAUCGGCGCC
CAGCGGAAGGCCCUGGAGAAGGUGCGGAAGGACGAGUACGAGAUC
GUGGGCCUGGCCGAGUGGUACGUGCCCGCCAUCGUGAUGUACCAG
GCCAUCCACAACAACUUCCACACCAAGCUGGAGUACAAGAGCGUG
AGCCGGGAGGAGAUGAUCGACUACCUGGAGAACAAGACCCUGAGC
UGGAACAGCAAGAACCCCGUGAGCAACGGCUACUGGAAGCGGAAG
AAGGACGACGAGCUGAAGAUCAUCUACAACGCCAUCAAGCUGAGC
GAGAAGGAGGGCAACAUCUUCGACAUCCGGGACCUGUACAAGCGG
ACCCUGAAGAACAUCGACCUGCUGACCUACAGCUUCCCCUGCCAG
GACCUGAGCCAGCAGGGCAUCCAGAAGGGCAUGAAGCGGGGCAGC
GGCACCCGGAGCGGCCUGCUGUGGGAGAUCGAGCGGGCCCUGGAC
AGCACCGAGAAGAACGACCUGCCCAAGUACCUGCUGAUGGAGAAC
GUGGGCGCCCUGCUGCACAAGAAGAACGAGGAGGAGCUGAACCAG
UGGAAGCAGAAGCUGGAGAGCCUGGGCUACCAGAACAGCAUCGAG
GUGCUGAACGCCGCCGACUUCGGCAGCAGCCAGGCCCGGCGGCGG
GUGUUCAUGAUCAGCACCCUGAACGAGUUCGUGGAGCUGCCCAAG
GGCGACAAGAAGCCCAAGAGCAUCAAGAAGGUGCUGAACAAGAUC
GUGAGCGAGAAGGACAUCCUGAACAACCUGCUGAAGUACAACCUG
ACCGAGUUCAAGAAAACCAAGAGCAACAUCAACAAGGCCAGCCUG
AUCGGCUACAGCAAGUUCAACAGCGAGGGCUACGUGUACGACCCC
GAGUUCACCGGCCCCACCCUGACCGCCAGCGGCGCCAACAGCCGG
AUCAAGAUCAAGGACGGCAGCAACAUCCGGAAGAUGAACAGCGAC
GAGACCUUCCUGUACAUCGGCUUCGACAGCCAGGACGGCAAGCGG
GUGAACGAGAUCGAGUUCCUGACCGAGAACCAGAAGAUCUUCGUG
UGCGGCAACAGCAUCAGCGUGGAGGUGCUGGAGGCCAUCAUCGAC
AAGAUCGGCGGCCCCAGCAGCGGCGGCAAGCGGCCCGCCGCCACC
AAGAAGGCCGGCCAGGCCAAGAAGAAGAAGGGCAGCUACCCCUAC
GACGUGCCCGACUACGCCUGAGCGGCCGCUUAAUUAAGCUGCCUU
CUGCGGGGCUUGCCUUCUGGCCAUGCCCUUCUUCUCUCCCUUGCA
CCUGUACCUCUUGGUCUUUGAAUAAAGCCUGAGUAGGAAGUCUAG
AAAAAAAAAAAAAAAAAAAAAAAAAAAAAAAAAAAAAAAAAAAAA
AAAAAAAAAAAAAAAAAAAAAAAAAAAAAAAAAAA

In some embodiments, a site-specific disrupting agent comprises the amino acid sequence of SEQ ID NOs: 203, 208, 73 or 74. In some embodiments, a site-specific disrupting agent described herein comprises an amino acid sequence of SEQ ID NO: 203, 208, 73 or 74, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### Sa-dCas9-MQ1 Protein Sequence:

(SEQ ID NO: 203)
MAPKKKRKVGIHGVPAAAKRNYILGLAIGITSVGYGIIDYETRDV
IDAGVRLFKEANVENNEGRRSKRGARRLKRRRRHRIQRVKKLLFD
YNLLTDHSELSGINPYEARVKGLSQKLSEEEFSAALLHLAKRRGV
HNVNEVEEDTGNELSTKEQISRNSKALEEKYVAELQLERLKKDGE
VRGSINRFKTSDYVKEAKQLLKVQKAYHQLDQSFIDTYIDLLETR
RTYYEGPGEGSPFGWKDIKEWYEMLMGHCTYFPEELRSVKYAYNA
DLYNALNDLNNLVITRDENEKLEYYEKFQIIENVFKQKKKPTLKQ
IAKEILVNEEDIKGYRVTSTGKPEFTNLKVYHDIKDITARKEIIE
NAELLDQIAKILTIYQSSEDIQEELTNLNSELTQEEIEQISNLKG
YTGTHNLSLKAINLILDELWHTNDNQIAIFNRLKLVPKKVDLSQQ
KEIPTTLVDDFILSPVVKRSFIQSIKVINAIIKKYGLPNDIIIEL
AREKNSKDAQKMINEMQKRNRQTNERIEEIIRTTGKENAKYLIEK
IKLHDMQEGKCLYSLEAIPLEDLLNNPFNYEVDAIIPRSVSFDNS
FNNKVLVKQEENSKKGNRTPFQYLSSSDSKISYETFKKHILNLAK
GKGRISKTKKEYLLEERDINRFSVQKDFINRNLVDTRYATRGLMN
LLRSYFRVNNLDVKVKSINGGFTSFLRRKWKFKKERNKGYKHHAE
DALIIANADFIFKEWKKLDKAKKVMENQMFEEKQAESMPEIETEQ
EYKEIFITPHQIKHIKDFKDYKYSHRVDKKPNRKLINDTLYSTRK
DDKGNTLIVNNLNGLYDKDNDKLKKLINKSPEKLLMYHHDPQTYQ
KLKLIMEQYGDEKNPLYKYYEETGNYLTKYSKKDNGPVIKKIKYY
GNKLNAHLDITDDYPNSRNKVVKLSLKPYRFDVYLDNGVYKFVTV
KNLDVIKKENYYEVNSKCYEEAKKLKKISNQAEFIASFYKNDLIK
INGELYRVIGVNNDLLNRIEVNMIDITYREYLENMNDKRPPHIIK
TIASKTQSIKKYSTDILGNLYEVKSKKHPQIIKKGKRPAATKKAG
QAKKKKARDSKVENKTKKLRVFEAFAGIGAQRKALEKVRKDEYEI
VGLAEWYVPAIVMYQAIHNNFHTKLEYKSVSREEMIDYLENKTLS
WNSKNPVSNGYWKRKKDDELKIIYNAIKLSEKEGNIFDIRDLYKR
TLKNIDLLTYSFPCQDLSQQGIQKGMKRGSGTRSGLLWEIERALD
STEKNDLPKYLLMENVGALLHKKNEEELNQWKQKLESLGYQNSIE
VLNAADFGSSQARRRVFMISTLNEFVELPKGDKKPKSIKKVLNKI
VSEKDILNNLLKYNLTEFKKTKSNINKASLIGYSKFNSEGYVYDP
EFTGPTLTASGANSRIKIKDGSNIRKMNSDETFLYIGFDSQDGKR
VNEIEFLTENQKIFVCGNSISVEVLEAIIDKIGGPSSGGKRPAAT
KKAGQAKKKKGSYPYDVPDYA

dCas9-MQ1 Protein Sequence (Corresponding to MR-28125):

(SEQ ID NO: 208)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDKRPAATKKAGQ
AKKKKARDSKVENKTKKLRVFEAFAGIGAQRKALEKVRKDEYEIV
GLAEWYVPAIVMYQAIHNNFHTKLEYKSVSREEMIDYLENKTLSW
NSKNPVSNGYWKRKKDDELKIIYNAIKLSEKEGNIFDIRDLYKRT
LKNIDLLTYSFPCQDLSQQGIQKGMKRGSGTRSGLLWEIERALDS
TEKNDLPKYLLMENVGALLHKKNEEELNQWKQKLESLGYQNSIEV
LNAADFGSSQARRRVFMISTLNEFVELPKGDKKPKSIKKVLNKIV
SEKDILNNLLKYNLTEFKKTKSNINKASLIGYSKFNSEGYVYDPE
FTGPTLTASGANSRIKIKDGSNIRKMNSDETFLYIGFDSQDGKRV
NEIEFLTENQKIFVCGNSISVEVLEAIIDKIGGPSSGGKRPAATK
KAGQAKKKKGSYPYDVPDYA

Sa-dCas9-MQ1 without HA Tag

(SEQ ID NO: 73)
MAPKKKRKVGIHGVPAAAKRNYILGLAIGITSVGYGIIDYETRDV
IDAGVRLFKEANVENNEGRRSKRGARRLKRRRRHRIQRVKKLLFD
YNLLTDHSELSGINPYEARVKGLSQKLSEEEFSAALLHLAKRRGV
HNVNEVEEDTGNELSTKEQISRNSKALEEKYVAELQLERLKKDGE
VRGSINRFKTSDYVKEAKQLLKVQKAYHQLDQSFIDTYIDLLETR
RTYYEGPGEGSPFGWKDIKEWYEMLMGHCTYFPEELRSVKYAYNA
DLYNALNDLNNLVITRDENEKLEYYEKFQIIENVFKQKKKPTLKQ
IAKEILVNEEDIKGYRVTSTGKPEFTNLKVYHDIKDITARKEIIE
NAELLDQIAKILTIYQSSEDIQEELTNLNSELTQEEIEQISNLKG
YTGTHNLSLKAINLILDELWHTNDNQIAIFNRLKLVPKKVDLSQQ
KEIPTTLVDDFILSPVVKRSFIQSIKVINAIIKKYGLPNDIIIEL
AREKNSKDAQKMINEMQKRNRQTNERIEEIIRTTGKENAKYLIEK
IKLHDMQEGKCLYSLEAIPLEDLLNNPFNYEVDAIIPRSVSFDNS
FNNKVLVKQEENSKKGNRTPFQYLSSSDSKISYETFKKHILNLAK
GKGRISKTKKEYLLEERDINRFSVQKDFINRNLVDTRYATRGLMN
LLRSYFRVNNLDVKVKSINGGFTSFLRRKWKFKKERNKGYKHHAE
DALIIANADFIFKEWKKLDKAKKVMENQMFEEKQAESMPEIETEQ
EYKEIFITPHQIKHIKDFKDYKYSHRVDKKPNRKLINDTLYSTRK
DDKGNTLIVNNLNGLYDKDNDKLKKLINKSPEKLLMYHHDPQTYQ
KLKLIMEQYGDEKNPLYKYYEETGNYLTKYSKKDNGPVIKKIKYY
GNKLNAHLDITDDYPNSRNKVVKLSLKPYRFDVYLDNGVYKFVTV
KNLDVIKKENYYEVNSKCYEEAKKLKKISNQAEFIASFYKNDLIK
INGELYRVIGVNNDLLNRIEVNMIDITYREYLENMNDKRPPHIIK
TIASKTQSIKKYSTDILGNLYEVKSKKHPQIIKKGKRPAATKKAG
QAKKKKARDSKVENKTKKLRVFEAFAGIGAQRKALEKVRKDEYEI
VGLAEWYVPAIVMYQAIHNNFHTKLEYKSVSREEMIDYLENKTLS
WNSKNPVSNGYWKRKKDDELKIIYNAIKLSEKEGNIFDIRDLYKR
TLKNIDLLTYSFPCQDLSQQGIQKGMKRGSGTRSGLLWEIERALD
STEKNDLPKYLLMENVGALLHKKNEEELNQWKQKLESLGYQNSIE
VLNAADFGSSQARRRVFMISTLNEFVELPKGDKKPKSIKKVLNKI
VSEKDILNNLLKYNLTEFKKTKSNINKASLIGYSKFNSEGYVYDP
EFTGPTLTASGANSRIKIKDGSNIRKMNSDETFLYIGFDSQDGKR
VNEIEFLTENQKIFVCGNSISVEVLEAIIDKIGGPSSGGKRPAAT
KKAGQAKKKKGS

dCas9-MQ1 without HA Tag

(SEQ ID NO: 74)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDKRPAATKKAGQ
AKKKKARDSKVENKTKKLRVFEAFAGIGAQRKALEKVRKDEYEIV
GLAEWYVPAIVMYQAIHNNFHTKLEYKSVSREEMIDYLENKTLSW
NSKNPVSNGYWKRKKDDELKIIYNAIKLSEKEGNIFDIRDLYKRT
LKNIDLLTYSFPCQDLSQQGIQKGMKRGSGTRSGLLWEIERALDS
TEKNDLPKYLLMENVGALLHKKNEEELNQWKQKLESLGYQNSIEV
LNAADFGSSQARRRVFMISTLNEFVELPKGDKKPKSIKKVLNKIV
SEKDILNNLLKYNLTEFKKTKSNINKASLIGYSKFNSEGYVYDPE
FTGPTLTASGANSRIKIKDGSNIRKMNSDETFLYIGFDSQDGKRV
NEIEFLTENQKIFVCGNSISVEVLEAIIDKIGGPSSGGKRPAATK
KAGQAKKKKGS

In some embodiments, a site-specific disrupting agent comprises a targeting moiety (e.g., comprising dCas9, e.g., an S. pyogenes dCas9), and an effector moiety comprising KRAB, e.g., a KRAB domain. In some embodiments, the site-specific disrupting agent is encoded by the nucleic acid sequence of SEQ ID NOs: 204 (e.g., a plasmid encoding the site-specific disrupting agent) and/or 205 (e.g., a nucleic acid (e.g., mRNA) encoding the site-specific disrupting agent). In some embodiments, a nucleic acid described herein comprises a nucleic acid sequence of SEQ ID NO. 204 or 205 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the targeting moiety is encoded by the nucleic acid sequence of SEQ ID NO. 8 and the effector moiety is encoded by the nucleic acid sequence of SEQ ID NO. 14.

### Sp-dCas9-KRAB PL-27687 Plasmid DNA Sequence:

(SEQ ID NO: 204)
TCGCGCGTTTCGGTGATGACGGTGAAAACCTCTGACACATGCAGC
TCCCGGAGACGGTCACAGCTTGTCTGTAAGCGGATGCCGGGAGCA
GACAAGCCCGTCAGGGCGCGTCAGCGGGTGTTGGCGGGTGTCGGG
GCTGGCTTAACTATGCGGCATCAGAGCAGATTGTACTGAGAGTGC
ACCATATGCGGTGTGAAATACCGCACAGATGCGTAAGGAGAAAAT
ACCGCATCAGGCGCCATTCGCCATTCAGGCTGCGCAACTGTTGGG
AAGGGCGATCGGTGCGGGCCTCTTCGCTATTACGCCAGCTGGCGA
AAGGGGGATGTGCTGCAAGGCGATTAAGTTGGGTAACGCCAGGGT
TTTCCCAGTCACGACGTTGTAAAACGACGGCCAGTGAATTGACGC
GTATTGGGATGGTACCTAATACGACTCACTATAAGGAAATAAGAG
AGAAAAGAAGAGTAAGAAGAAATATAAGAGCCACCATGGCCCCCA
AGAAGAAGCGGAAGGTGGGCATCCACGGCGTGCCCGCCGCCGACA
AGAAGTACAGCATCGGCCTGGCCATCGGCACCAACAGCGTGGGCT
GGGCCGTGATCACCGACGAGTACAAGGTGCCCAGCAAGAAGTTCA
AGGTGCTGGGCAACACCGACCGGCACAGCATCAAGAAGAACCTGA
TCGGCGCCCTGCTGTTCGACAGCGGCGAGACCGCCGAGGCCACCC
GGCTGAAGCGGACCGCCCGGCGGCGGTACACCCGGCGGAAGAACC
GGATCTGCTACCTGCAGGAGATCTTCAGCAACGAGATGGCCAAGG
TGGACGACAGCTTCTTCCACCGGCTGGAGGAGAGCTTCCTGGTGG
AGGAGGACAAGAAGCACGAGCGGCACCCCATCTTCGGCAACATCG
TGGACGAGGTGGCCTACCACGAGAAGTACCCCACCATCTACCACC
TGCGGAAGAAGCTGGTGGACAGCACCGACAAGGCCGACCTGCGGC
TGATCTACCTGGCCCTGGCCCACATGATCAAGTTCCGGGGCCACT
TCCTGATCGAGGGCGACCTGAACCCCGACAACAGCGACGTGGACA
AGCTGTTCATCCAGCTGGTGCAGACCTACAACCAGCTGTTCGAGG
AGAACCCCATCAACGCCAGCGGCGTGGACGCCAAGGCCATCCTGA
GCGCCCGGCTGAGCAAGAGCCGGCGGCTGGAGAACCTGATCGCCC
AGCTGCCCGGCGAGAAGAAGAACGGCCTGTTCGGCAACCTGATCG
CCCTGAGCCTGGGCCTGACCCCCAACTTCAAGAGCAACTTCGACC
TGGCCGAGGACGCCAAGCTGCAGCTGAGCAAGGACACCTACGACG
ACGACCTGGACAACCTGCTGGCCCAGATCGGCGACCAGTACGCCG
ACCTGTTCCTGGCCGCCAAGAACCTGAGCGACGCCATCCTGCTGA
GCGACATCCTGCGGGTGAACACCGAGATCACCAAGGCCCCCCTGA
GCGCCAGCATGATCAAGCGGTACGACGAGCACCACCAGGACCTGA
CCCTGCTGAAGGCCCTGGTGCGGCAGCAGCTGCCCGAGAAGTACA
AGGAGATCTTCTTCGACCAGAGCAAGAACGGCTACGCCGGCTACA
TCGACGGCGGCGCCAGCCAGGAGGAGTTCTACAAGTTCATCAAGC
CCATCCTGGAGAAGATGGACGGCACCGAGGAGCTGCTGGTGAAGC
TGAACCGGGAGGACCTGCTGCGGAAGCAGCGGACCTTCGACAACG
GCAGCATCCCCCACCAGATCCACCTGGGCGAGCTGCACGCCATCC
TGCGGCGGCAGGAGGACTTCTACCCCTTCCTGAAGGACAACCGGG
AGAAGATCGAGAAGATCCTGACCTTCCGGATCCCCTACTACGTGG
GCCCCCTGGCCCGGGGCAACAGCCGGTTCGCCTGGATGACCCGGa
aatccGAGGAGACCATCACCCCCTGGAACTTCGAGGAGGTGGTGG
ACAAGGGCGCCAGCGCCCAGAGCTTCATCGAGCGGATGACCAACT
TCGACAAGAACCTGCCCAACGAGAAGGTGCTGCCCAAGCACAGCC
TGCTGTACGAGTACTTCACCGTGTACAACGAGCTGACCAAGGTGA
AGTACGTGACCGAGGGCATGCGGAAGCCCGCCTTCCTGAGCGGCG
AGCAGAAGAAGGCCATCGTGGACCTGCTGTTCAAGACCAACCGGA
AGGTGACCGTGAAGCAGCTGAAGGAGGACTACTTCAAGAAGATCG
AGTGCTTCGACAGCGTGGAGATCAGCGGCGTGGAGGACCGGTTCA
ACGCCAGCCTGGGCACCTACCACGACCTGCTGAAGATCATCAAGG
ACAAGGACTTCCTGGACAACGAGGAGAACGAGGACATCCTGGAGG
ACATCGTGCTGACCCTGACCCTGTTCGAGGACCGGGAGATGATCG
AGGAGCGGCTGAAaACCTACGCCCACCTGTTCGACGACAAGGTGA
TGAAGCAGCTGAAGCGGCGGCGGTACACCGGCTGGGGCCGGCTGA
GCCGGAAGCTGATCAACGGCATCCGGGACAAGCAGAGCGGCAAGA
CCATCCTGGACTTCCTGaaatccGACGGCTTCGCCAACCGGAACT
TCATGCAGCTGATCCACGACGACAGCCTGACCTTCAAGGAGGACA
TCCAGAAGGCCCAGGTGAGCGGCCAGGGCGACAGCCTGCACGAGC
ACATCGCCAACCTGGCCGGCAGCCCCGCCATCAAGAAGGGCATCC
TGCAGACCGTGAAGGTGGTGGACGAGCTGGTGAAGGTGATGGGCC
GGCACAAGCCCGAGAACATCGTGATCGAGATGGCCCGGGAGAACC
AGACCACCCAGAAGGGCCAGAAGAACAGCCGGGAGCGGATGAAGC
GGATCGAGGAGGGCATCAAGGAGCTGGGCAGCCAGATCCTGAAGG
AGCACCCCGTGGAGAACACCCAGCTGCAGAACGAGAAGCTGTACC
TGTACTACCTGCAGAACGGCCGGGACATGTACGTGGACCAGGAGC
TGGACATCAACCGGCTGAGCGACTACGACGTGGCCGCCATCGTGC
CCCAGAGCTTCCTGAAGGACGACAGCATCGACAACAAGGTGCTGA
CCCGGAGCGACAAGGCCCGGGGCAAGAGCGACAACGTGCCCAGCG
AGGAGGTGGTGAAGAAGATGAAGAACTACTGGCGGCAGCTGCTGA
ACGCCAAGCTGATCACCCAGCGGAAGTTCGACAACCTGACCAAGG
CCGAGCGGGGCGGCCTGAGCGAGCTGGACAAGGCCGGCTTCATCA
AGCGGCAGCTGGTGGAGACCCGGCAGATCACCAAGCACGTGGCCC
AGATCCTGGACAGCCGGATGAACACCAAGTACGACGAGAACGACA
AGCTGATCCGGGAGGTGAAGGTGATCACCCTGaaatccAAGCTGG
TGAGCGACTTCCGGAAGGACTTCCAGTTCTACAAGGTGCGGGAGA
TCAACAACTACCACCACGCCCACGACGCCTACCTGAACGCCGTGG
TGGGCACCGCCCTGATCAAGAAGTACCCCAAGCTGGAGAGCGAGT
TCGTGTACGGCGACTACAAGGTGTACGACGTGCGGAAGATGATCG
CCAAGAGCGAGCAGGAGATCGGCAAGGCCACCGCCAAGTACTTCT
TCTACAGCAACATCATGAACTTCTTCAAGACCGAGATCACCCTGG
CCAACGGCGAGATCCGGAAGCGGCCCCTGATCGAGACCAACGGCG
AGACCGGCGAGATCGTGTGGGACAAGGGCCGGGACTTCGCCACCG
TGCGGAAGGTGCTGAGCATGCCCCAGGTGAACATCGTGAAGAAaA
CCGAGGTGCAGACCGGCGGCTTCAGCAAGGAGAGCATCCTGCCCA
AGCGGAACAGCGACAAGCTGATCGCCCGGAAGAAGGACTGGGACC
CCAAGAAGTACGGCGGCTTCGACAGCCCCACCGTGGCCTACAGCG
TGCTGGTGGTGGCCAAGGTGGAGAAGGGCAAGAGCAAGAAGCTGa
aatccGTGAAGGAGCTGCTGGGCATCACCATCATGGAGCGGAGCA
GCTTCGAGAAGAACCCCATCGACTTCCTGGAGGCCAAGGGCTACA
AGGAGGTGAAGAAGGACCTGATCATCAAGCTGCCCAAGTACAGCC
TGTTCGAGCTGGAGAACGGCCGGAAGCGGATGCTGGCCAGCGCCG
GCGAGCTGCAGAAGGGCAACGAGCTGGCCCTGCCCAGCAAGTACG
TGAACTTCCTGTACCTGGCCAGCCACTACGAGAAGCTGAAGGGCA
GCCCCGAGGACAACGAGCAGAAGCAGCTGTTCGTGGAGCAGCACA
AGCACTACCTGGACGAGATCATCGAGCAGATCAGCGAGTTCAGCA
AGCGGGTGATCCTGGCCGACGCCAACCTGGACAAGGTGCTGAGCG
CCTACAACAAGCACCGGGACAAGCCCATCCGGGAGCAGGCCGAGA
ACATCATCCACCTGTTCACCCTGACCAACCTGGGCGCCCCCGCCG
CCTTCAAGTACTTCGACACCACCATCGACCGGAAGCGGTACACCA
GCACCAAGGAGGTGCTGGACGCCACCCTGATCCACCAGAGCATCA
CCGGCCTGTACGAGACCCGGATCGACCTGAGCCAGCTGGGCGGCG
ACAAGCGGCCCGCCGCCACCAAGAAGGCCGGCCAGGCCAAGAAGA
AGAAGGCCAGCGACGCCAAGAGCCTGACCGCCTGGAGCCGGACCC
TGGTGACCTTCAAGGACGTGTTCGTGGACTTCACCCGGGAGGAGT
GGAAGCTGCTGGACACCGCCCAGCAGATCCTGTACCGGAACGTGA
TGCTGGAGAACTACAAGAACCTGGTGAGCCTGGGCTACCAGCTGA
CCAAGCCCGACGTGATCCTGCGGCTGGAGAAGGGCGAGGAGCCCT
GGCTGGTGGAGCGGGAGATCCACCAGGAGACCCACCCCGACAGCG
AGACCGCCTTCGAGATCAAGAGCAGCGTGAGCGGCGGCAAGCGGC
CCGCCGCCACCAAGAAGGCCGGCCAGGCCAAGAAGAAGAAGGGCA
GCTACCCCTACGACGTGCCCGACTACGCCTGAGcggccgcttaat
taagctgccttctgcggggcttgccttctggccatgcccttcttc
tctcccttgcacctgtacctcttggtctttgaataaagcctgagt
aggaagtctagaaaaaaaaaaaaaaaaaaaaaaaaaaaaaaaaaa
aaaaaaaaaaaaaaaaaaaaaaaaaaaaaaaaaaaaaattgtctt
ctcatcgcctgcagATCCCAATGGCGCGCCGAGCTTGGCTCGAGC
ATGGTCATAGCTGTTTCCTGTGTGAAATTGTTATCCGCTCACAAT
TCCACACAACATACGAGCCGGAAGCATAAAGTGTAAAGCCTGGGG
TGCCTAATGAGTGAGCTAACTCACATTAATTGCGTTGCGCTCACT
GCCCGCTTTCCAGTCGGGAAACCTGTCGTGCCAGCTGCATTAATG
AATCGGCCAACGCGCGGGGAGAGGCGGTTTGCGTATTGGGCGCTC
TTCCGCTTCCTCGCTCACTGACTCGCTGCGCTCGGTCGTTCGGCT
GCGGCGAGCGGTATCAGCTCACTCAAAGGCGGTAATACGGTTATC
CACAGAATCAGGGGATAACGCAGGAAAGAACATGTGAGCAAAAGG
CCAGCAAAAGGCCAGGAACCGTAAAAAGGCCGCGTTGCTGGCGTT
TTTCCATAGGCTCCGCCCCCCTGACGAGCATCACAAAAATCGACG
CTCAAGTCAGAGGTGGCGAAACCCGACAGGACTATAAAGATACCA
GGCGTTTCCCCCTGGAAGCTCCCTCGTGCGCTCTCCTGTTCCGAC
CCTGCCGCTTACCGGATACCTGTCCGCCTTTCTCCCTTCGGGAAG
CGTGGCGCTTTCTCATAGCTCACGCTGTAGGTATCTCAGTTCGGT
GTAGGTCGTTCGCTCCAAGCTGGGCTGTGTGCACGAACCCCCCGT
TCAGCCCGACCGCTGCGCCTTATCCGGTAACTATCGTCTTGAGTC
CAACCCGGTAAGACACGACTTATCGCCACTGGCAGCAGCCACTGG
TAACAGGATTAGCAGAGCGAGGTATGTAGGCGGTGCTACAGAGTT
CTTGAAGTGGTGGCCTAACTACGGCTACACTAGAAGAACAGTATT
TGGTATCTGCGCTCTGCTGAAGCCAGTTACCTTCGGAAAAAGAGT
TGGTAGCTCTTGATCCGGCAAACAAACCACCGCTGGTAGCGGTGG
TTTTTTTGTTTGCAAGCAGCAGATTACGCGCAGAAAAAAAGGATC
TCAAGAAGATCCTTTGATCTTTTCTACGGGGTCTGACGCTCAGTG
GAACGAAAACTCACGTTAAGGGATTTTGGTCATGAGATTATCAAA
AAGGATCTTCACCTAGATCCTTTTAAATTAAAAATGAAGTTTTAA
ATCAATCTAAAGTATATATGAGTAAACTTGGTCTGACAGTTAGAA
AAACTCATCGAGCATCAAATGAAACTGCAATTTATTCATATCAGG
ATTATCAATACCATATTTTTGAAAAAGCCGTTTCTGTAATGAAGG
AGAAAACTCACCGAGGCAGTTCCATAGGATGGCAAGATCCTGGTA
TCGGTCTGCGATTCCGACTCGTCCAACATCAATACAACCTATTAA
TTTCCCCTCGTCAAAAATAAGGTTATCAAGTGAGAAATCACCATG
AGTGACGACTGAATCCGGTGAGAATGGCAAAAGTTTATGCATTTC
TTTCCAGACTTGTTCAACAGGCCAGCCATTACGCTCGTCATCAAA
ATCACTCGCATCAACCAAACCGTTATTCATTCGTGATTGCGCCTG
AGCGAGACGAAATACGCGATCGCTGTTAAAAGGACAATTACAAAC
AGGAATCGAATGCAACCGGCGCAGGAACACTGCCAGCGCATCAAC
AATATTTTCACCTGAATCAGGATATTCTTCTAATACCTGGAATGC
TGTTTTCCCAGGGATCGCAGTGGTGAGTAACCATGCATCATCAGG
AGTACGGATAAAATGCTTGATGGTCGGAAGAGGCATAAATTCCGT
CAGCCAGTTTAGTCTGACCATCTCATCTGTAACATCATTGGCAAC
GCTACCTTTGCCATGTTTCAGAAACAACTCTGGCGCATCGGGCTT
CCCATACAATCGATAGATTGTCGCACCTGATTGCCCGACATTATC
GCGAGCCCATTTATACCCATATAAATCAGCATCCATGTTGGAATT
TAATCGCGGCCTAGAGCAAGACGTTTCCCGTTGAATATGGCTCAT
ACTCTTCCTTTTTCAATATTATTGAAGCATTTATCAGGGTTATTG
TCTCATGAGCGGATACATATTTGAATGTATTTAGAAAAATAAACA
AATAGGGGTTCCGCGCACATTTCCCCGAAAAGTGCCACCTGACGT
CTAAGAAACCATTATTATCATGACATTAACCTATAAAAATAGGCG
TATCACGAGGCCCTTTCGTC

Sp-dCas9-KRAB (MR-28122) Expressed mRNA Sequence:

(SEQ ID NO: 205)
AGGAAATAAGAGAGAAAAGAAGAGTAAGAAGAAATATAAGAGCCA
CCATGGCCCCCAAGAAGAAGCGGAAGGTGGGCATCCACGGCGTGC
CCGCCGCCGACAAGAAGTACAGCATCGGCCTGGCCATCGGCACCA
ACAGCGTGGGCTGGGCCGTGATCACCGACGAGTACAAGGTGCCCA
GCAAGAAGTTCAAGGTGCTGGGCAACACCGACCGGCACAGCATCA
AGAAGAACCTGATCGGCGCCCTGCTGTTCGACAGCGGCGAGACCG
CCGAGGCCACCCGGCTGAAGCGGACCGCCCGGCGGCGGTACACCC
GGCGGAAGAACCGGATCTGCTACCTGCAGGAGATCTTCAGCAACG
AGATGGCCAAGGTGGACGACAGCTTCTTCCACCGGCTGGAGGAGA
GCTTCCTGGTGGAGGAGGACAAGAAGCACGAGCGGCACCCCATCT
TCGGCAACATCGTGGACGAGGTGGCCTACCACGAGAAGTACCCCA
CCATCTACCACCTGCGGAAGAAGCTGGTGGACAGCACCGACAAGG
CCGACCTGCGGCTGATCTACCTGGCCCTGGCCCACATGATCAAGT
TCCGGGGCCACTTCCTGATCGAGGGCGACCTGAACCCCGACAACA
GCGACGTGGACAAGCTGTTCATCCAGCTGGTGCAGACCTACAACC
AGCTGTTCGAGGAGAACCCCATCAACGCCAGCGGCGTGGACGCCA
AGGCCATCCTGAGCGCCCGGCTGAGCAAGAGCCGGCGGCTGGAGA
ACCTGATCGCCCAGCTGCCCGGCGAGAAGAAGAACGGCCTGTTCG
GCAACCTGATCGCCCTGAGCCTGGGCCTGACCCCCAACTTCAAGA
GCAACTTCGACCTGGCCGAGGACGCCAAGCTGCAGCTGAGCAAGG
ACACCTACGACGACGACCTGGACAACCTGCTGGCCCAGATCGGCG
ACCAGTACGCCGACCTGTTCCTGGCCGCCAAGAACCTGAGCGACG
CCATCCTGCTGAGCGACATCCTGCGGGTGAACACCGAGATCACCA
AGGCCCCCCTGAGCGCCAGCATGATCAAGCGGTACGACGAGCACC
ACCAGGACCTGACCCTGCTGAAGGCCCTGGTGCGGCAGCAGCTGC
CCGAGAAGTACAAGGAGATCTTCTTCGACCAGAGCAAGAACGGCT
ACGCCGGCTACATCGACGGCGGCGCCAGCCAGGAGGAGTTCTACA
AGTTCATCAAGCCCATCCTGGAGAAGATGGACGGCACCGAGGAGC
TGCTGGTGAAGCTGAACCGGGAGGACCTGCTGCGGAAGCAGCGGA
CCTTCGACAACGGCAGCATCCCCCACCAGATCCACCTGGGCGAGC
TGCACGCCATCCTGCGGCGGCAGGAGGACTTCTACCCCTTCCTGA
AGGACAACCGGGAGAAGATCGAGAAGATCCTGACCTTCCGGATCC
CCTACTACGTGGGCCCCCTGGCCCGGGGCAACAGCCGGTTCGCCT
GGATGACCCGGaaatccGAGGAGACCATCACCCCCTGGAACTTCG
AGGAGGTGGTGGACAAGGGCGCCAGCGCCCAGAGCTTCATCGAGC
GGATGACCAACTTCGACAAGAACCTGCCCAACGAGAAGGTGCTGC
CCAAGCACAGCCTGCTGTACGAGTACTTCACCGTGTACAACGAGC
TGACCAAGGTGAAGTACGTGACCGAGGGCATGCGGAAGCCCGCCT
TCCTGAGCGGCGAGCAGAAGAAGGCCATCGTGGACCTGCTGTTCA
AGACCAACCGGAAGGTGACCGTGAAGCAGCTGAAGGAGGACTACT
TCAAGAAGATCGAGTGCTTCGACAGCGTGGAGATCAGCGGCGTGG
AGGACCGGTTCAACGCCAGCCTGGGCACCTACCACGACCTGCTGA
AGATCATCAAGGACAAGGACTTCCTGGACAACGAGGAGAACGAGG
ACATCCTGGAGGACATCGTGCTGACCCTGACCCTGTTCGAGGACC
GGGAGATGATCGAGGAGCGGCTGAAaACCTACGCCCACCTGTTCG
ACGACAAGGTGATGAAGCAGCTGAAGCGGCGGCGGTACACCGGCT
GGGGCCGGCTGAGCCGGAAGCTGATCAACGGCATCCGGGACAAGC
AGAGCGGCAAGACCATCCTGGACTTCCTGaaatccGACGGCTTCG
CCAACCGGAACTTCATGCAGCTGATCCACGACGACAGCCTGACCT
TCAAGGAGGACATCCAGAAGGCCCAGGTGAGCGGCCAGGGCGACA
GCCTGCACGAGCACATCGCCAACCTGGCCGGCAGCCCCGCCATCA
AGAAGGGCATCCTGCAGACCGTGAAGGTGGTGGACGAGCTGGTGA
AGGTGATGGGCCGGCACAAGCCCGAGAACATCGTGATCGAGATGG
CCCGGGAGAACCAGACCACCCAGAAGGGCCAGAAGAACAGCCGGG
AGCGGATGAAGCGGATCGAGGAGGGCATCAAGGAGCTGGGCAGCC
AGATCCTGAAGGAGCACCCCGTGGAGAACACCCAGCTGCAGAACG
AGAAGCTGTACCTGTACTACCTGCAGAACGGCCGGGACATGTACG
TGGACCAGGAGCTGGACATCAACCGGCTGAGCGACTACGACGTGG
CCGCCATCGTGCCCCAGAGCTTCCTGAAGGACGACAGCATCGACA
ACAAGGTGCTGACCCGGAGCGACAAGGCCCGGGGCAAGAGCGACA
ACGTGCCCAGCGAGGAGGTGGTGAAGAAGATGAAGAACTACTGGC
GGCAGCTGCTGAACGCCAAGCTGATCACCCAGCGGAAGTTCGACA
ACCTGACCAAGGCCGAGCGGGGCGGCCTGAGCGAGCTGGACAAGG
CCGGCTTCATCAAGCGGCAGCTGGTGGAGACCCGGCAGATCACCA
AGCACGTGGCCCAGATCCTGGACAGCCGGATGAACACCAAGTACG
ACGAGAACGACAAGCTGATCCGGGAGGTGAAGGTGATCACCCTGa
aatccAAGCTGGTGAGCGACTTCCGGAAGGACTTCCAGTTCTACA
AGGTGCGGGAGATCAACAACTACCACCACGCCCACGACGCCTACC
TGAACGCCGTGGTGGGCACCGCCCTGATCAAGAAGTACCCCAAGC
TGGAGAGCGAGTTCGTGTACGGCGACTACAAGGTGTACGACGTGC
GGAAGATGATCGCCAAGAGCGAGCAGGAGATCGGCAAGGCCACCG
CCAAGTACTTCTTCTACAGCAACATCATGAACTTCTTCAAGACCG
AGATCACCCTGGCCAACGGCGAGATCCGGAAGCGGCCCCTGATCG
AGACCAACGGCGAGACCGGCGAGATCGTGTGGGACAAGGGCCGGG
ACTTCGCCACCGTGCGGAAGGTGCTGAGCATGCCCCAGGTGAACA
TCGTGAAGAAaACCGAGGTGCAGACCGGCGGCTTCAGCAAGGAGA
GCATCCTGCCCAAGCGGAACAGCGACAAGCTGATCGCCCGGAAGA
AGGACTGGGACCCCAAGAAGTACGGCGGCTTCGACAGCCCCACCG
TGGCCTACAGCGTGCTGGTGGTGGCCAAGGTGGAGAAGGGCAAGA
GCAAGAAGCTGaaatccGTGAAGGAGCTGCTGGGCATCACCATCA
TGGAGCGGAGCAGCTTCGAGAAGAACCCCATCGACTTCCTGGAGG
CCAAGGGCTACAAGGAGGTGAAGAAGGACCTGATCATCAAGCTGC
CCAAGTACAGCCTGTTCGAGCTGGAGAACGGCCGGAAGCGGATGC
TGGCCAGCGCCGGCGAGCTGCAGAAGGGCAACGAGCTGGCCCTGC
CCAGCAAGTACGTGAACTTCCTGTACCTGGCCAGCCACTACGAGA
AGCTGAAGGGCAGCCCCGAGGACAACGAGCAGAAGCAGCTGTTCG
TGGAGCAGCACAAGCACTACCTGGACGAGATCATCGAGCAGATCA
GCGAGTTCAGCAAGCGGGTGATCCTGGCCGACGCCAACCTGGACA
AGGTGCTGAGCGCCTACAACAAGCACCGGGACAAGCCCATCCGGG
AGCAGGCCGAGAACATCATCCACCTGTTCACCCTGACCAACCTGG
GCGCCCCCGCCGCCTTCAAGTACTTCGACACCACCATCGACCGGA
AGCGGTACACCAGCACCAAGGAGGTGCTGGACGCCACCCTGATCC
ACCAGAGCATCACCGGCCTGTACGAGACCCGGATCGACCTGAGCC
AGCTGGGCGGCGACAAGCGGCCCGCCGCCACCAAGAAGGCCGGCC
AGGCCAAGAAGAAGAAGGCCAGCGACGCCAAGAGCCTGACCGCCT
GGAGCCGGACCCTGGTGACCTTCAAGGACGTGTTCGTGGACTTCA
CCCGGGAGGAGTGGAAGCTGCTGGACACCGCCCAGCAGATCCTGT
ACCGGAACGTGATGCTGGAGAACTACAAGAACCTGGTGAGCCTGG
GCTACCAGCTGACCAAGCCCGACGTGATCCTGCGGCTGGAGAAGG
GCGAGGAGCCCTGGCTGGTGGAGCGGGAGATCCACCAGGAGACCC
ACCCCGACAGCGAGACCGCCTTCGAGATCAAGAGCAGCGTGAGCG
GCGGCAAGCGGCCCGCCGCCACCAAGAAGGCCGGCCAGGCCAAGA
AGAAGAAGGGCAGCTACCCCTACGACGTGCCCGACTACGCCTGAG
cggccgcttaattaagctgccttctgcggggcttgccttctggcc
atgcccttcttctctcccttgcacctgtacctcttggtctttgaa
taaagcctgagtaggaagtctagaaaaaaaaaaaaaaaaaaaaaa
aaaaaaaaaaaaaaaaaaaaaaaaaaaaaaaaaaaaaaaaaaaaa
aaaaaaaaaaaaa

In some embodiments, a site-specific disrupting agent comprises the amino acid sequence of SEQ ID NOs: 206 or 75. In some embodiments, a nucleic acid described herein comprises a nucleic acid sequence of SEQ ID NO: 206 or 75 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### Sp-dCas9-KRAB Protein Sequence:

(SEQ ID NO: 206)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDKRPAATKKAGQ
AKKKKASDAKSLTAWSRTLVTFKDVFVDFTREEWKLLDTAQQILY
RNVMLENYKNLVSLGYQLTKPDVILRLEKGEEPWLVEREIHQETH
PDSETAFEIKSSVSGGKRPAATKKAGQAKKKKGSYPYDVPDYA

Sp-dCas9-KRAB Protein Sequence without HA Tag:

(SEQ ID NO: 75)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDKRPAATKKAGQ
AKKKKASDAKSLTAWSRTLVTFKDVFVDFTREEWKLLDTAQQILY
RNVMLENYKNLVSLGYQLTKPDVILRLEKGEEPWLVEREIHQETH
PDSETAFEIKSSVSGGKRPAATKKAGQAKKKKGS

In some embodiments, a site-specific disrupting agent comprises a targeting moiety (e.g., comprising dCas9, e.g., an S. aureus dCas9) and an effector moiety comprising EZH2. In some embodiments, a site-specific disrupting agent comprises a targeting moiety comprising dCas9, e.g., an S. pyogenes dCas9, e.g., mutant S. pyogenes Cas9, e.g., Cas9m4 and an effector moiety comprising EZH2. In some embodiments, the site-specific disrupting agent is encoded by the nucleic acid sequence of SEQ ID NO: 209 (e.g., mRNA encoding the site-specific disrupting agent). In some embodiments, a nucleic acid described herein comprises a nucleic acid sequence of SEQ ID NO: 209 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the targeting moiety is encoded by the nucleic acid sequence of SEQ ID NO: 8 and/or the effector moiety is encoded by the nucleic acid sequence of SEQ ID NO: 18.

EZH2-dCas9 mRNA (MR28938)

(SEQ ID NO: 209)
AGGAAAUAAGAGAGAAAAGAAGAGUAAGAAGAAAUAUAAGAGCCA
CCAUGGCCCCCAAGAAGAAGCGGAAGGUGGGCGGCAGCGGCGGCA
GCGGCCAGACCGGCAAGAAGAGCGAGAAGGGCCCCGUGUGCUGGC
GGAAGCGGGUGAAGAGCGAGUACAUGCGGCUGCGGCAGCUGAAGC
GGUUCCGGCGGGCCGACGAGGUGAAGAGCAUGUUCAGCAGCAACC
GGCAGAAGAUCCUGGAGCGGACCGAGAUCCUGAACCAGGAGUGGA
AGCAGCGGCGAAUCCAGCCCGUGCACAUCCUGACCAGCGUGAGCA
GCCUGCGGGGCACCCGGGAGUGCAGCGUGACCAGCGACCUGGACU
UCCCCACCCAGGUGAUCCCCCUAAAGACCCUGAACGCCGUGGCCA
GCGUGCCCAUCAUGUACAGCUGGAGCCCCCUGCAGCAGAACUUCA
UGGUGGAGGACGAGACCGUGCUGCACAACAUCCCCUACAUGGGCG
ACGAGGUGCUGGACCAGGACGGCACCUUCAUCGAGGAGCUGAUCA
AGAACUACGACGGCAAGGUGCACGGCGACCGGGAGUGCGGCUUCA
UCAACGACGAGAUCUUCGUGGAGCUGGUGAACGCCCUGGGCCAGU
ACAACGACGACGACGACGACGACGACGGCGACGACCCCGAGGAGC
GGGAGGAGAAGCAGAAGGACCUGGAGGACCACCGGGACGACAAGG
AGAGCCGGCCCCCCCGGAAGUUCCCCAGCGACAAGAUCUUCGAGG
CCAUCAGCAGCAUGUUCCCCGACAAGGGCACCGCCGAGGAGCUGA
AGGAGAAGUACAAGGAGCUGACCGAGCAGCAGCUGCCCGGCGCCC
UGCCCCCCGAGUGCACCCCCAACAUCGACGGCCCCAACGCCAAGA
GCGUGCAGCGGGAGCAGAGCCUGCACAGCUUCCACACCCUGUUCU
GCCGGCGGUGCUUCAAGUACGACUGCUUCCUGCACCCCUUCCACG
CCACCCCCAACACCUACAAGCGGAAGAACACCGAGACCGCCCUGG
ACAACAAGCCCUGCGGCCCCCAGUGCUACCAGCACCUGGAGGGCG
CCAAGGAGUUCGCCGCCGCCCUGACCGCCGAGCGGAUCAAGACCC
CCCCCAAGCGGCCCGGCGGCCGGCGGCGGGGCCGGCUGCCCAACA
ACAGCAGCCGGCCCAGCACCCCCACCAUCAACGUGCUGGAGAGCA
AGGACACCGACAGCGACCGGGAGGCCGGCACCGAGACCGGCGGCG
AGAACAACGACAAGGAGGAGGAGGAGAAGAAGGACGAGACCAGCA
GCAGCAGCGAGGCCAACAGCCGGUGCCAGACCCCCAUCAAGAUGA
AGCCCAACAUCGAGCCCCCCGAGAACGUGGAGUGGAGCGGCGCCG
AGGCCAGCAUGUUCCGGGUGCUGAUCGGCACCUACUACGACAACU
UCUGCGCCAUCGCCCGGCUGAUCGGCACCAAGACCUGCCGGCAGG
UGUACGAGUUCCGGGUGAAGGAGAGCAGCAUCAUCGCCCCCGCCC
CCGCCGAGGACGUGGACACCCCCCCCCGGAAGAAGAAGCGGAAGC
ACCGGCUGUGGGCCGCCCACUGCCGGAAGAUCCAGCUGAAGAAGG
ACGGCAGCAGCAACCACGUGUACAACUACCAGCCCUGCGACCACC
CCCGGCAGCCCUGCGACAGCAGCUGCCCCUGCGUGAUCGCCCAGA
ACUUCUGCGAGAAGUUCUGCCAGUGCAGCAGCGAGUGCCAGAACC
GGUUCCCCGGCUGCCGGUGCAAGGCCCAGUGCAACACCAAGCAGU
GCCCCUGCUACCUGGCCGUGCGGGAGUGCGACCCCGACCUGUGCC
UGACCUGCGGCGCCGCCGACCACUGGGACAGCAAGAACGUGAGCU
GCAAGAACUGCAGCAUCCAGCGGGGCAGCAAGAAGCACCUGCUGC
UGGCCCCCAGCGACGUGGCCGGCUGGGGCAUCUUCAUCAAGGACC
CCGUGCAGAAGAACGAGUUCAUCAGCGAGUACUGCGGCGAGAUCA
UCAGCCAGGACGAGGCCGACCGGCGGGGCAAGGUGUACGACAAGU
ACAUGUGCAGCUUCCUGUUCAACCUGAACAACGACUUCGUGGUGG
ACGCCACCCGGAAGGGCAACAAGAUCCGGUUCGCCAACCACAGCG
UGAACCCCAACUGCUACGCCAAGGUGAUGAUGGUGAACGGCGACC
ACCGGAUCGGCAUCUUCGCCAAGCGGGCCAUCCAGACCGGCGAGG
AGCUGUUCUUCGACUACCGGUACAGCCAGGCCGACGCCCUGAAGU
ACGUGGGCAUCGAGCGGGAGAUGGAGAUCCCCAGCACCGGCGGCA
GCGGCGGCAGCGGCGGCAGCGGCGGCAGCGGCGGCAGCGGCCGAC
CCGACAAGAAGUACAGCAUCGGCCUGGCCAUCGGCACCAACAGCG
UGGGCUGGGCCGUGAUCACCGACGAGUACAAGGUGCCCAGCAAGA
AGUUCAAGGUGCUGGGCAACACCGACCGGCACAGCAUCAAGAAGA
ACCUGAUCGGCGCCCUGCUGUUCGACAGCGGCGAGACCGCCGAGG
CCACCCGGCUGAAGCGGACCGCCCGGCGGCGGUACACCCGGCGGA
AGAACCGGAUCUGCUACCUGCAGGAGAUCUUCAGCAACGAGAUGG
CCAAGGUGGACGACAGCUUCUUCCACCGGCUGGAGGAGAGCUUCC
UGGUGGAGGAGGACAAGAAGCACGAGCGGCACCCCAUCUUCGGCA
ACAUCGUGGACGAGGUGGCCUACCACGAGAAGUACCCCACCAUCU
ACCACCUGCGGAAGAAGCUGGUGGACAGCACCGACAAGGCCGACC
UGCGGCUGAUCUACCUGGCCCUGGCCCACAUGAUCAAGUUCCGGG
GCCACUUCCUGAUCGAGGGCGACCUGAACCCCGACAACAGCGACG
UGGACAAGCUGUUCAUCCAGCUGGUGCAGACCUACAACCAGCUGU
UCGAGGAGAACCCCAUCAACGCCAGCGGCGUGGACGCCAAGGCCA
UCCUGAGCGCCCGGCUGAGCAAGAGCCGGCGGCUGGAGAACCUGA
UCGCCCAGCUGCCCGGCGAGAAGAAGAACGGCCUGUUCGGCAACC
UGAUCGCCCUGAGCCUGGGCCUGACCCCCAACUUCAAGAGCAACU
UCGACCUGGCCGAGGACGCCAAGCUGCAGCUGAGCAAGGACACCU
ACGACGACGACCUGGACAACCUGCUGGCCCAGAUCGGCGACCAGU
ACGCCGACCUGUUCCUGGCCGCCAAGAACCUGAGCGACGCCAUCC
UGCUGAGCGACAUCCUGCGGGUGAACACCGAGAUCACCAAGGCCC
CCCUGAGCGCCAGCAUGAUCAAGCGGUACGACGAGCACCACCAGG
ACCUGACCCUGCUGAAGGCCCUGGUGCGGCAGCAGCUGCCCGAGA
AGUACAAGGAGAUCUUCUUCGACCAGAGCAAGAACGGCUACGCCG
GCUACAUCGACGGCGGCGCCAGCCAGGAGGAGUUCUACAAGUUCA
UCAAGCCCAUCCUGGAGAAGAUGGACGGCACCGAGGAGCUGCUGG
UGAAGCUGAACCGGGAGGACCUGCUGCGGAAGCAGCGGACCUUCG
ACAACGGCAGCAUCCCCCACCAGAUCCACCUGGGCGAGCUGCACG
CCAUCCUGCGGCGGCAGGAGGACUUCUACCCCUUCCUGAAGGACA
ACCGGGAGAAGAUCGAGAAGAUCCUGACCUUCCGGAUCCCCUACU
ACGUGGGCCCCCUGGCCCGGGGCAACAGCCGGUUCGCCUGGAUGA
CCCGGAAAUCCGAGGAGACCAUCACCCCCUGGAACUUCGAGGAGG
UGGUGGACAAGGGCGCCAGCGCCCAGAGCUUCAUCGAGCGGAUGA
CCAACUUCGACAAGAACCUGCCCAACGAGAAGGUGCUGCCCAAGC
ACAGCCUGCUGUACGAGUACUUCACCGUGUACAACGAGCUGACCA
AGGUGAAGUACGUGACCGAGGGCAUGCGGAAGCCCGCCUUCCUGA
GCGGCGAGCAGAAGAAGGCCAUCGUGGACCUGCUGUUCAAGACCA
ACCGGAAGGUGACCGUGAAGCAGCUGAAGGAGGACUACUUCAAGA
AGAUCGAGUGCUUCGACAGCGUGGAGAUCAGCGGCGUGGAGGACC
GGUUCAACGCCAGCCUGGGCACCUACCACGACCUGCUGAAGAUCA
UCAAGGACAAGGACUUCCUGGACAACGAGGAGAACGAGGACAUCC
UGGAGGACAUCGUGCUGACCCUGACCCUGUUCGAGGACCGGGAGA
UGAUCGAGGAGCGGCUGAAAACCUACGCCCACCUGUUCGACGACA
AGGUGAUGAAGCAGCUGAAGCGGCGGCGGUACACCGGCUGGGGCC
GGCUGAGCCGGAAGCUGAUCAACGGCAUCCGGGACAAGCAGAGCG
GCAAGACCAUCCUGGACUUCCUGAAAUCCGACGGCUUCGCCAACC
GGAACUUCAUGCAGCUGAUCCACGACGACAGCCUGACCUUCAAGG
AGGACAUCCAGAAGGCCCAGGUGAGCGGCCAGGGCGACAGCCUGC
ACGAGCACAUCGCCAACCUGGCCGGCAGCCCCGCCAUCAAGAAGG
GCAUCCUGCAGACCGUGAAGGUGGUGGACGAGCUGGUGAAGGUGA
UGGGCCGGCACAAGCCCGAGAACAUCGUGAUCGAGAUGGCCCGGG
AGAACCAGACCACCCAGAAGGGCCAGAAGAACAGCCGGGAGCGGA
UGAAGCGGAUCGAGGAGGGCAUCAAGGAGCUGGGCAGCCAGAUCC
UGAAGGAGCACCCCGUGGAGAACACCCAGCUGCAGAACGAGAAGC
UGUACCUGUACUACCUGCAGAACGGCCGGGACAUGUACGUGGACC
AGGAGCUGGACAUCAACCGGCUGAGCGACUACGACGUGGCCGCCA
UCGUGCCCCAGAGCUUCCUGAAGGACGACAGCAUCGACAACAAGG
UGCUGACCCGGAGCGACAAGGCCCGGGGCAAGAGCGACAACGUGC
CCAGCGAGGAGGUGGUGAAGAAGAUGAAGAACUACUGGCGGCAGC
UGCUGAACGCCAAGCUGAUCACCCAGCGGAAGUUCGACAACCUGA
CCAAGGCCGAGCGGGGCGGCCUGAGCGAGCUGGACAAGGCCGGCU
UCAUCAAGCGGCAGCUGGUGGAGACCCGGCAGAUCACCAAGCACG
UGGCCCAGAUCCUGGACAGCCGGAUGAACACCAAGUACGACGAGA
ACGACAAGCUGAUCCGGGAGGUGAAGGUGAUCACCCUGAAAUCCA
AGCUGGUGAGCGACUUCCGGAAGGACUUCCAGUUCUACAAGGUGC
GGGAGAUCAACAACUACCACCACGCCCACGACGCCUACCUGAACG
CCGUGGUGGGCACCGCCCUGAUCAAGAAGUACCCCAAGCUGGAGA
GCGAGUUCGUGUACGGCGACUACAAGGUGUACGACGUGCGGAAGA
UGAUCGCCAAGAGCGAGCAGGAGAUCGGCAAGGCCACCGCCAAGU
ACUUCUUCUACAGCAACAUCAUGAACUUCUUCAAGACCGAGAUCA
CCCUGGCCAACGGCGAGAUCCGGAAGCGGCCCCUGAUCGAGACCA
ACGGCGAGACCGGCGAGAUCGUGUGGGACAAGGGCCGGGACUUCG
CCACCGUGCGGAAGGUGCUGAGCAUGCCCCAGGUGAACAUCGUGA
AGAAAACCGAGGUGCAGACCGGCGGCUUCAGCAAGGAGAGCAUCC
UGCCCAAGCGGAACAGCGACAAGCUGAUCGCCCGGAAGAAGGACU
GGGACCCCAAGAAGUACGGCGGCUUCGACAGCCCCACCGUGGCCU
ACAGCGUGCUGGUGGUGGCCAAGGUGGAGAAGGGCAAGAGCAAGA
AGCUGAAAUCCGUGAAGGAGCUGCUGGGCAUCACCAUCAUGGAGC
GGAGCAGCUUCGAGAAGAACCCCAUCGACUUCCUGGAGGCCAAGG
GCUACAAGGAGGUGAAGAAGGACCUGAUCAUCAAGCUGCCCAAGU
ACAGCCUGUUCGAGCUGGAGAACGGCCGGAAGCGGAUGCUGGCCA
GCGCCGGCGAGCUGCAGAAGGGCAACGAGCUGGCCCUGCCCAGCA
AGUACGUGAACUUCCUGUACCUGGCCAGCCACUACGAGAAGCUGA
AGGGCAGCCCCGAGGACAACGAGCAGAAGCAGCUGUUCGUGGAGC
AGCACAAGCACUACCUGGACGAGAUCAUCGAGCAGAUCAGCGAGU
UCAGCAAGCGGGUGAUCCUGGCCGACGCCAACCUGGACAAGGUGC
UGAGCGCCUACAACAAGCACCGGGACAAGCCCAUCCGGGAGCAGG
CCGAGAACAUCAUCCACCUGUUCACCCUGACCAACCUGGGCGCCC
CCGCCGCCUUCAAGUACUUCGACACCACCAUCGACCGGAAGCGGU
ACACCAGCACCAAGGAGGUGCUGGACGCCACCCUGAUCCACCAGA
GCAUCACCGGCCUGUACGAGACCCGGAUCGACCUGAGCCAGCUGG
GCGGCGACAGCGGCGGCAAGCGGCCCGCCGCCACCAAGAAGGCCG
GCCAGGCCAAGAAGAAGAAGGGCAGCUACCCCUACGACGUGCCCG
ACUACGCCUGAGCGGCCGCUUAAUUAAGCUGCCUUCUGCGGGGCU
UGCCUUCUGGCCAUGCCCUUCUUCUCUCCCUUGCACCUGUACCUC
UUGGUCUUUGAAUAAAGCCUGAGUAGGAAGUCUAGAAAAAAAAAA
AAAAAAAAAAAAAAAAAAAAAAAAAAAAAAAAAAAAAAAAAAAAA
AAAAAAAAAAAAAAAAAAAAAAAAA

In some embodiments, a site-specific disrupting agent comprises the amino acid sequence of SEQ ID NOs: 210 or 76. In some embodiments, a nucleic acid described herein comprises a nucleic acid sequence of SEQ ID NO: 210 or 76 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### EZH2-dCas9 Protein Sequence (Corresponding to MR-28938)

(SEQ ID NO: 210)
MAPKKKRKVGGSGGSGQTGKKSEKGPVCWRKRVKSEYMRLRQLKR
FRRADEVKSMFSSNRQKILERTEILNQEWKQRRIQPVHILTSVSS
LRGTRECSVTSDLDFPTQVIPLKTLNAVASVPIMYSWSPLQQNFM
VEDETVLHNIPYMGDEVLDQDGTFIEELIKNYDGKVHGDRECGFI
NDEIFVELVNALGQYNDDDDDDDGDDPEEREEKQKDLEDHRDDKE
SRPPRKFPSDKIFEAISSMFPDKGTAEELKEKYKELTEQQLPGAL
PPECTPNIDGPNAKSVQREQSLHSFHTLFCRRCFKYDCFLHPFHA
TPNTYKRKNTETALDNKPCGPQCYQHLEGAKEFAAALTAERIKTP
PKRPGGRRRGRLPNNSSRPSTPTINVLESKDTDSDREAGTETGGE
NNDKEEEEKKDETSSSSEANSRCQTPIKMKPNIEPPENVEWSGAE
ASMFRVLIGTYYDNFCAIARLIGTKTCRQVYEFRVKESSIIAPAP
AEDVDTPPRKKKRKHRLWAAHCRKIQLKKDGSSNHVYNYQPCDHP
RQPCDSSCPCVIAQNFCEKFCQCSSECQNRFPGCRCKAQCNTKQC
PCYLAVRECDPDLCLTCGAADHWDSKNVSCKNCSIQRGSKKHLLL
APSDVAGWGIFIKDPVQKNEFISEYCGEIISQDEADRRGKVYDKY
MCSFLFNLNNDFVVDATRKGNKIRFANHSVNPNCYAKVMMVNGDH
RIGIFAKRAIQTGEELFFDYRYSQADALKYVGIEREMEIPSTGGS
GGSGGSGGSGGSGRPDKKYSIGLAIGTNSVGWAVITDEYKVPSKK
FKVLGNTDRHSIKKNLIGALLFDSGETAEATRLKRTARRRYTRRK
NRICYLQEIFSNEMAKVDDSFFHRLEESFLVEEDKKHERHPIFGN
IVDEVAYHEKYPTIYHLRKKLVDSTDKADLRLIYLALAHMIKFRG
HFLIEGDLNPDNSDVDKLFIQLVQTYNQLFEENPINASGVDAKAI
LSARLSKSRRLENLIAQLPGEKKNGLFGNLIALSLGLTPNFKSNF
DLAEDAKLQLSKDTYDDDLDNLLAQIGDQYADLFLAAKNLSDAIL
LSDILRVNTEITKAPLSASMIKRYDEHHQDLTLLKALVRQQLPEK
YKEIFFDQSKNGYAGYIDGGASQEEFYKFIKPILEKMDGTEELLV
KLNREDLLRKQRTFDNGSIPHQIHLGELHAILRRQEDFYPFLKDN
REKIEKILTFRIPYYVGPLARGNSRFAWMTRKSEETITPWNFEEV
VDKGASAQSFIERMTNFDKNLPNEKVLPKHSLLYEYFTVYNELTK
VKYVTEGMRKPAFLSGEQKKAIVDLLFKTNRKVTVKQLKEDYFKK
IECFDSVEISGVEDRFNASLGTYHDLLKIIKDKDFLDNEENEDIL
EDIVLTLTLFEDREMIEERLKTYAHLFDDKVMKQLKRRRYTGWGR
LSRKLINGIRDKQSGKTILDFLKSDGFANRNFMQLIHDDSLTFKE
DIQKAQVSGQGDSLHEHIANLAGSPAIKKGILQTVKVVDELVKVM
GRHKPENIVIEMARENQTTQKGQKNSRERMKRIEEGIKELGSQIL
KEHPVENTQLQNEKLYLYYLQNGRDMYVDQELDINRLSDYDVAAI
VPQSFLKDDSIDNKVLTRSDKARGKSDNVPSEEVVKKMKNYWRQL
LNAKLITQRKFDNLTKAERGGLSELDKAGFIKRQLVETRQITKHV
AQILDSRMNTKYDENDKLIREVKVITLKSKLVSDFRKDFQFYKVR
EINNYHHAHDAYLNAVVGTALIKKYPKLESEFVYGDYKVYDVRKM
IAKSEQEIGKATAKYFFYSNIMNFFKTEITLANGEIRKRPLIETN
GETGEIVWDKGRDFATVRKVLSMPQVNIVKKTEVQTGGFSKESIL
PKRNSDKLIARKKDWDPKKYGGFDSPTVAYSVLVVAKVEKGKSKK
LKSVKELLGITIMERSSFEKNPIDFLEAKGYKEVKKDLIIKLPKY
SLFELENGRKRMLASAGELQKGNELALPSKYVNFLYLASHYEKLK
GSPEDNEQKQLFVEQHKHYLDEIIEQISEFSKRVILADANLDKVL
SAYNKHRDKPIREQAENIIHLFTLTNLGAPAAFKYFDTTIDRKRY
TSTKEVLDATLIHQSITGLYETRIDLSQLGGDSGGKRPAATKKAG
QAKKKKGSYPYDVPDYA

EZH2-dCas9 without HA Tag

(SEQ ID NO: 76)
MAPKKKRKVGGSGGSGQTGKKSEKGPVCWRKRVKSEYMRLRQLKR
FRRADEVKSMFSSNRQKILERTEILNQEWKQRRIQPVHILTSVSS
LRGTRECSVTSDLDFPTQVIPLKTLNAVASVPIMYSWSPLQQNFM
VEDETVLHNIPYMGDEVLDQDGTFIEELIKNYDGKVHGDRECGFI
NDEIFVELVNALGQYNDDDDDDDGDDPEEREEKQKDLEDHRDDKE
SRPPRKFPSDKIFEAISSMFPDKGTAEELKEKYKELTEQQLPGAL
PPECTPNIDGPNAKSVQREQSLHSFHTLFCRRCFKYDCFLHPFHA
TPNTYKRKNTETALDNKPCGPQCYQHLEGAKEFAAALTAERIKTP
PKRPGGRRRGRLPNNSSRPSTPTINVLESKDTDSDREAGTETGGE
NNDKEEEEKKDETSSSSEANSRCQTPIKMKPNIEPPENVEWSGAE
ASMFRVLIGTYYDNFCAIARLIGTKTCRQVYEFRVKESSIIAPAP
AEDVDTPPRKKKRKHRLWAAHCRKIQLKKDGSSNHVYNYQPCDHP
RQPCDSSCPCVIAQNFCEKFCQCSSECQNRFPGCRCKAQCNTKQC
PCYLAVRECDPDLCLTCGAADHWDSKNVSCKNCSIQRGSKKHLLL
APSDVAGWGIFIKDPVQKNEFISEYCGEIISQDEADRRGKVYDKY
MCSFLFNLNNDFVVDATRKGNKIRFANHSVNPNCYAKVMMVNGDH
RIGIFAKRAIQTGEELFFDYRYSQADALKYVGIEREMEIPSTGGS
GGSGGSGGSGGSGRPDKKYSIGLAIGTNSVGWAVITDEYKVPSKK
FKVLGNTDRHSIKKNLIGALLFDSGETAEATRLKRTARRRYTRRK
NRICYLQEIFSNEMAKVDDSFFHRLEESFLVEEDKKHERHPIFGN
IVDEVAYHEKYPTIYHLRKKLVDSTDKADLRLIYLALAHMIKFRG
HFLIEGDLNPDNSDVDKLFIQLVQTYNQLFEENPINASGVDAKAI
LSARLSKSRRLENLIAQLPGEKKNGLFGNLIALSLGLTPNFKSNF
DLAEDAKLQLSKDTYDDDLDNLLAQIGDQYADLFLAAKNLSDAIL
LSDILRVNTEITKAPLSASMIKRYDEHHQDLTLLKALVRQQLPEK
YKEIFFDQSKNGYAGYIDGGASQEEFYKFIKPILEKMDGTEELLV
KLNREDLLRKQRTFDNGSIPHQIHLGELHAILRRQEDFYPFLKDN
REKIEKILTFRIPYYVGPLARGNSRFAWMTRKSEETITPWNFEEV
VDKGASAQSFIERMTNFDKNLPNEKVLPKHSLLYEYFTVYNELTK
VKYVTEGMRKPAFLSGEQKKAIVDLLFKTNRKVTVKQLKEDYFKK
IECFDSVEISGVEDRFNASLGTYHDLLKIIKDKDFLDNEENEDIL
EDIVLTLTLFEDREMIEERLKTYAHLFDDKVMKQLKRRRYTGWGR
LSRKLINGIRDKQSGKTILDFLKSDGFANRNFMQLIHDDSLTFKE
DIQKAQVSGQGDSLHEHIANLAGSPAIKKGILQTVKVVDELVKVM
GRHKPENIVIEMARENQTTQKGQKNSRERMKRIEEGIKELGSQIL
KEHPVENTQLQNEKLYLYYLQNGRDMYVDQELDINRLSDYDVAAI
VPQSFLKDDSIDNKVLTRSDKARGKSDNVPSEEVVKKMKNYWRQL
LNAKLITQRKFDNLTKAERGGLSELDKAGFIKRQLVETRQITKHV
AQILDSRMNTKYDENDKLIREVKVITLKSKLVSDFRKDFQFYKVR
EINNYHHAHDAYLNAVVGTALIKKYPKLESEFVYGDYKVYDVRKM
IAKSEQEIGKATAKYFFYSNIMNFFKTEITLANGEIRKRPLIETN
GETGEIVWDKGRDFATVRKVLSMPQVNIVKKTEVQTGGFSKESIL
PKRNSDKLIARKKDWDPKKYGGFDSPTVAYSVLVVAKVEKGKSKK
LKSVKELLGITIMERSSFEKNPIDFLEAKGYKEVKKDLIIKLPKY
SLFELENGRKRMLASAGELQKGNELALPSKYVNFLYLASHYEKLK
GSPEDNEQKQLFVEQHKHYLDEIIEQISEFSKRVILADANLDKVL
SAYNKHRDKPIREQAENIIHLFTLTNLGAPAAFKYFDTTIDRKRY
TSTKEVLDATLIHQSITGLYETRIDLSQLGGDSGGKRPAATKKAG
QAKKKKGS

In some embodiments, a site-specific disrupting agent comprises a targeting moiety (e.g., comprising dCas9, e.g., an S. aureus dCas9), and an effector moiety comprising DNMT3 (e.g., DNMT3a/3L). In some embodiments, a site-specific disrupting agent comprises a targeting moiety comprising dCas9, e.g., an S. pyogenes dCas9, e.g., mutant S. pyogenes Cas9, e.g., Cas9m4 and an effector moiety comprising DNMT3 (e.g., DNMT3a/3L). In some embodiments, the site-specific disrupting agent is encoded by the nucleic acid sequence of SEQ ID NO: 211 (e.g., mRNA encoding the site-specific disrupting agent). In some embodiments, a nucleic acid described herein comprises a nucleic acid sequence of SEQ ID NO: 211 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT3A/3L mRNA (MR-29414)

(SEQ ID NO: 211)
AGGAAAUAAGAGAGAAAAGAAGAGUAAGAAGAAAUAUAAGAGCCA
CCAUGGCCCCCAAGAAGAAGCGGAAGGUGGGCAUCCACGGCGUGC
CCGCCGCCGACAAGAAGUACAGCAUCGGCCUGGCCAUCGGCACCA
ACAGCGUGGGCUGGGCCGUGAUCACCGACGAGUACAAGGUGCCCA
GCAAGAAGUUCAAGGUGCUGGGCAACACCGACCGGCACAGCAUCA
AGAAGAACCUGAUCGGCGCCCUGCUGUUCGACAGCGGCGAGACCG
CCGAGGCCACCCGGCUGAAGCGGACCGCCCGGCGGCGGUACACCC
GGCGGAAGAACCGGAUCUGCUACCUGCAGGAGAUCUUCAGCAACG
AGAUGGCCAAGGUGGACGACAGCUUCUUCCACCGGCUGGAGGAGA
GCUUCCUGGUGGAGGAGGACAAGAAGCACGAGCGGCACCCCAUCU
UCGGCAACAUCGUGGACGAGGUGGCCUACCACGAGAAGUACCCCA
CCAUCUACCACCUGCGGAAGAAGCUGGUGGACAGCACCGACAAGG
CCGACCUGCGGCUGAUCUACCUGGCCCUGGCCCACAUGAUCAAGU
UCCGGGGCCACUUCCUGAUCGAGGGCGACCUGAACCCCGACAACA
GCGACGUGGACAAGCUGUUCAUCCAGCUGGUGCAGACCUACAACC
AGCUGUUCGAGGAGAACCCCAUCAACGCCAGCGGCGUGGACGCCA
AGGCCAUCCUGAGCGCCCGGCUGAGCAAGAGCCGGCGGCUGGAGA
ACCUGAUCGCCCAGCUGCCCGGCGAGAAGAAGAACGGCCUGUUCG
GCAACCUGAUCGCCCUGAGCCUGGGCCUGACCCCCAACUUCAAGA
GCAACUUCGACCUGGCCGAGGACGCCAAGCUGCAGCUGAGCAAGG
ACACCUACGACGACGACCUGGACAACCUGCUGGCCCAGAUCGGCG
ACCAGUACGCCGACCUGUUCCUGGCCGCCAAGAACCUGAGCGACG
CCAUCCUGCUGAGCGACAUCCUGCGGGUGAACACCGAGAUCACCA
AGGCCCCCCUGAGCGCCAGCAUGAUCAAGCGGUACGACGAGCACC
ACCAGGACCUGACCCUGCUGAAGGCCCUGGUGCGGCAGCAGCUGC
CCGAGAAGUACAAGGAGAUCUUCUUCGACCAGAGCAAGAACGGCU
ACGCCGGCUACAUCGACGGCGGCGCCAGCCAGGAGGAGUUCUACA
AGUUCAUCAAGCCCAUCCUGGAGAAGAUGGACGGCACCGAGGAGC
UGCUGGUGAAGCUGAACCGGGAGGACCUGCUGCGGAAGCAGCGGA
CCUUCGACAACGGCAGCAUCCCCCACCAGAUCCACCUGGGCGAGC
UGCACGCCAUCCUGCGGCGGCAGGAGGACUUCUACCCCUUCCUGA
AGGACAACCGGGAGAAGAUCGAGAAGAUCCUGACCUUCCGGAUCC
CCUACUACGUGGGCCCCCUGGCCCGGGGCAACAGCCGGUUCGCCU
GGAUGACCCGGAAAUCCGAGGAGACCAUCACCCCCUGGAACUUCG
AGGAGGUGGUGGACAAGGGCGCCAGCGCCCAGAGCUUCAUCGAGC
GGAUGACCAACUUCGACAAGAACCUGCCCAACGAGAAGGUGCUGC
CCAAGCACAGCCUGCUGUACGAGUACUUCACCGUGUACAACGAGC
UGACCAAGGUGAAGUACGUGACCGAGGGCAUGCGGAAGCCCGCCU
UCCUGAGCGGCGAGCAGAAGAAGGCCAUCGUGGACCUGCUGUUCA
AGACCAACCGGAAGGUGACCGUGAAGCAGCUGAAGGAGGACUACU
UCAAGAAGAUCGAGUGCUUCGACAGCGUGGAGAUCAGCGGCGUGG
AGGACCGGUUCAACGCCAGCCUGGGCACCUACCACGACCUGCUGA
AGAUCAUCAAGGACAAGGACUUCCUGGACAACGAGGAGAACGAGG
ACAUCCUGGAGGACAUCGUGCUGACCCUGACCCUGUUCGAGGACC
GGGAGAUGAUCGAGGAGCGGCUGAAAACCUACGCCCACCUGUUCG
ACGACAAGGUGAUGAAGCAGCUGAAGCGGCGGCGGUACACCGGCU
GGGGCCGGCUGAGCCGGAAGCUGAUCAACGGCAUCCGGGACAAGC
AGAGCGGCAAGACCAUCCUGGACUUCCUGAAAUCCGACGGCUUCG
CCAACCGGAACUUCAUGCAGCUGAUCCACGACGACAGCCUGACCU
UCAAGGAGGACAUCCAGAAGGCCCAGGUGAGCGGCCAGGGCGACA
GCCUGCACGAGCACAUCGCCAACCUGGCCGGCAGCCCCGCCAUCA
AGAAGGGCAUCCUGCAGACCGUGAAGGUGGUGGACGAGCUGGUGA
AGGUGAUGGGCCGGCACAAGCCCGAGAACAUCGUGAUCGAGAUGG
CCCGGGAGAACCAGACCACCCAGAAGGGCCAGAAGAACAGCCGGG
AGCGGAUGAAGCGGAUCGAGGAGGGCAUCAAGGAGCUGGGCAGCC
AGAUCCUGAAGGAGCACCCCGUGGAGAACACCCAGCUGCAGAACG
AGAAGCUGUACCUGUACUACCUGCAGAACGGCCGGGACAUGUACG
UGGACCAGGAGCUGGACAUCAACCGGCUGAGCGACUACGACGUGG
CCGCCAUCGUGCCCCAGAGCUUCCUGAAGGACGACAGCAUCGACA
ACAAGGUGCUGACCCGGAGCGACAAGGCCCGGGGCAAGAGCGACA
ACGUGCCCAGCGAGGAGGUGGUGAAGAAGAUGAAGAACUACUGGC
GGCAGCUGCUGAACGCCAAGCUGAUCACCCAGCGGAAGUUCGACA
ACCUGACCAAGGCCGAGCGGGGCGGCCUGAGCGAGCUGGACAAGG
CCGGCUUCAUCAAGCGGCAGCUGGUGGAGACCCGGCAGAUCACCA
AGCACGUGGCCCAGAUCCUGGACAGCCGGAUGAACACCAAGUACG
ACGAGAACGACAAGCUGAUCCGGGAGGUGAAGGUGAUCACCCUGA
AAUCCAAGCUGGUGAGCGACUUCCGGAAGGACUUCCAGUUCUACA
AGGUGCGGGAGAUCAACAACUACCACCACGCCCACGACGCCUACC
UGAACGCCGUGGUGGGCACCGCCCUGAUCAAGAAGUACCCCAAGC
UGGAGAGCGAGUUCGUGUACGGCGACUACAAGGUGUACGACGUGC
GGAAGAUGAUCGCCAAGAGCGAGCAGGAGAUCGGCAAGGCCACCG
CCAAGUACUUCUUCUACAGCAACAUCAUGAACUUCUUCAAGACCG
AGAUCACCCUGGCCAACGGCGAGAUCCGGAAGCGGCCCCUGAUCG
AGACCAACGGCGAGACCGGCGAGAUCGUGUGGGACAAGGGCCGGG
ACUUCGCCACCGUGCGGAAGGUGCUGAGCAUGCCCCAGGUGAACA
UCGUGAAGAAAACCGAGGUGCAGACCGGCGGCUUCAGCAAGGAGA
GCAUCCUGCCCAAGCGGAACAGCGACAAGCUGAUCGCCCGGAAGA
AGGACUGGGACCCCAAGAAGUACGGCGGCUUCGACAGCCCCACCG
UGGCCUACAGCGUGCUGGUGGUGGCCAAGGUGGAGAAGGGCAAGA
GCAAGAAGCUGAAAUCCGUGAAGGAGCUGCUGGGCAUCACCAUCA
UGGAGCGGAGCAGCUUCGAGAAGAACCCCAUCGACUUCCUGGAGG
CCAAGGGCUACAAGGAGGUGAAGAAGGACCUGAUCAUCAAGCUGC
CCAAGUACAGCCUGUUCGAGCUGGAGAACGGCCGGAAGCGGAUGC
UGGCCAGCGCCGGCGAGCUGCAGAAGGGCAACGAGCUGGCCCUGC
CCAGCAAGUACGUGAACUUCCUGUACCUGGCCAGCCACUACGAGA
AGCUGAAGGGCAGCCCCGAGGACAACGAGCAGAAGCAGCUGUUCG
UGGAGCAGCACAAGCACUACCUGGACGAGAUCAUCGAGCAGAUCA
GCGAGUUCAGCAAGCGGGUGAUCCUGGCCGACGCCAACCUGGACA
AGGUGCUGAGCGCCUACAACAAGCACCGGGACAAGCCCAUCCGGG
AGCAGGCCGAGAACAUCAUCCACCUGUUCACCCUGACCAACCUGG
GCGCCCCCGCCGCCUUCAAGUACUUCGACACCACCAUCGACCGGA
AGCGGUACACCAGCACCAAGGAGGUGCUGGACGCCACCCUGAUCC
ACCAGAGCAUCACCGGCCUGUACGAGACCCGGAUCGACCUGAGCC
AGCUGGGCGGCGACAGCGCCGGCGGCGGCGGCAGCGGCGGCGGCG
GCAGCGGCGGCGGCGGCAGCGGCCCCAAGAAGAAGCGGAAGGUGG
CCGCCGCCGGCAGCAACCACGACCAGGAGUUCGACCCCCCCAAGG
UGUACCCCCCCGUGCCCGCCGAGAAGCGGAAGCCCAUCCGGGUGC
UGAGCCUGUUCGACGGCAUCGCCACCGGCCUGCUGGUGCUGAAGG
ACCUGGGCAUCCAGGUGGACCGGUACAUCGCCAGCGAGGUGUGCG
AGGACAGCAUCACCGUGGGCAUGGUGCGGCACCAGGGCAAGAUCA
UGUACGUGGGCGACGUGCGGAGCGUGACCCAGAAGCACAUCCAGG
AGUGGGGCCCCUUCGACCUGGUGAUCGGCGGCAGCCCCUGCAACG
ACCUGAGCAUCGUGAACCCCGCCCGGAAGGGCCUGUACGAGGGCA
CCGGCCGGCUGUUCUUCGAGUUCUACCGGCUGCUGCACGACGCCC
GGCCCAAGGAGGGCGACGACCGGCCCUUCUUCUGGCUGUUCGAGA
ACGUGGUGGCCAUGGGCGUGAGCGACAAGCGGGACAUCAGCCGGU
UCCUGGAGAGCAACCCCGUGAUGAUCGACGCCAAGGAGGUGAGCG
CCGCCCACCGGGCCCGGUACUUCUGGGGCAACCUGCCCGGCAUGA
ACCGGCCCCUGGCCAGCACCGUGAACGACAAGCUGGAGCUGCAGG
AGUGCCUGGAGCACGGCCGGAUCGCCAAGUUCAGCAAGGUGCGGA
CCAUCACCACCCGGAGCAACAGCAUCAAGCAGGGCAAGGACCAGC
ACUUCCCCGUGUUCAUGAACGAGAAGGAGGACAUCCUGUGGUGCA
CCGAGAUGGAGCGGGUGUUCGGCUUCCCCGUGCACUACACCGACG
UGAGCAACAUGAGCCGGCUGGCCCGGCAGCGGCUGCUGGGCCGGA
GCUGGAGCGUGCCCGUGAUCCGGCACCUGUUCGCCCCCCUGAAGG
AGUACUUCGCCUGCGUGAGCAGCGGCAACAGCAACGCCAACAGCC
GGGGCCCCAGCUUCAGCAGCGGCCUGGUGCCCCUGAGCCUGCGGG
GCAGCCACAUGAAUCCUCUGGAGAUGUUCGAGACAGUGCCCGUGU
GGAGAAGGCAACCCGUGAGGGUGCUGAGCCUCUUCGAGGACAUUA
AGAAGGAGCUGACCUCUCUGGGCUUUCUGGAAUCCGGCAGCGACC
CCGGCCAGCUGAAACACGUGGUGGACGUGACCGACACAGUGAGGA
AGGACGUGGAAGAGUGGGGCCCCUUUGACCUCGUGUAUGGAGCCA
CACCUCCUCUCGGCCACACAUGCGAUAGGCCUCCCAGCUGGUAUC
UCUUCCAGUUCCACAGACUGCUCCAGUACGCCAGACCUAAGCCCG
GCAGCCCCAGACCCUUCUUCUGGAUGUUCGUGGACAAUCUGGUGC
UGAACAAGGAGGAUCUGGAUGUGGCCAGCAGAUUUCUGGAGAUGG
AACCCGUGACAAUCCCCGACGUGCAUGGCGGCUCUCUGCAGAACG
CCGUGAGAGUGUGGUCCAACAUCCCCGCCAUUAGAAGCAGACACU
GGGCUCUGGUGAGCGAGGAGGAACUGUCUCUGCUGGCCCAGAAUA
AGCAGUCCUCCAAGCUGGCCGCCAAGUGGCCCACCAAGCUGGUGA
AGAACUGCUUUCUGCCUCUGAGGGAGUAUUUCAAGUAUUUCAGCA
CCGAACUGACCAGCAGCCUGAGCGGCGGCAAGCGGCCCGCCGCCA
CCAAGAAGGCCGGCCAGGCCAAGAAGAAGAAGGGCAGCUACCCCU
ACGACGUGCCCGACUACGCCUGAGCGGCCGCUUAAUUAAGCUGCC
UUCUGCGGGGCUUGCCUUCUGGCCAUGCCCUUCUUCUCUCCCUUG
CACCUGUACCUCUUGGUCUUUGAAUAAAGCCUGAGUAGGAAGUCU
AGAAAAAAAAAAAAAAAAAAAAAAAAAAAAAAAAAAAAAAAAAAA
AAAAAAAAAAAAAAAAAAAAAAAAAAAAAAAAAAAAA

In some embodiments, a site-specific disrupting agent comprises the amino acid sequence of SEQ ID NOs: 211 or 77. In some embodiments, a construct described herein comprises an amino acid sequence of SEQ ID NO: 211 or 77 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT3a/3L Protein Sequence (Corresponding to MR-29414)

(SEQ ID NO: 212)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDSAGGGGSGGGG
SGGGGSGPKKKRKVAAAGSNHDQEFDPPKVYPPVPAEKRKPIRVL
SLFDGIATGLLVLKDLGIQVDRYIASEVCEDSITVGMVRHQGKIM
YVGDVRSVTQKHIQEWGPFDLVIGGSPCNDLSIVNPARKGLYEGT
GRLFFEFYRLLHDARPKEGDDRPFFWLFENVVAMGVSDKRDISRF
LESNPVMIDAKEVSAAHRARYFWGNLPGMNRPLASTVNDKLELQE
CLEHGRIAKFSKVRTITTRSNSIKQGKDQHFPVFMNEKEDILWCT
EMERVFGFPVHYTDVSNMSRLARQRLLGRSWSVPVIRHLFAPLKE
YFACVSSGNSNANSRGPSFSSGLVPLSLRGSHMNPLEMFETVPVW
RRQPVRVLSLFEDIKKELTSLGFLESGSDPGQLKHVVDVTDTVRK
DVEEWGPFDLVYGATPPLGHTCDRPPSWYLFQFHRLLQYARPKPG
SPRPFFWMFVDNLVLNKEDLDVASRFLEMEPVTIPDVHGGSLQNA
VRVWSNIPAIRSRHWALVSEEELSLLAQNKQSSKLAAKWPTKLVK
NCFLPLREYFKYFSTELTSSLSGGKRPAATKKAGQAKKKKGSYPY
DVPDYA

dCas-DNMT3a/3L (h) without HA Tag

(SEQ ID NO: 77)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDSAGGGGSGGGG
SGGGGSGPKKKRKVAAAGSNHDQEFDPPKVYPPVPAEKRKPIRVL
SLFDGIATGLLVLKDLGIQVDRYIASEVCEDSITVGMVRHQGKIM
YVGDVRSVTQKHIQEWGPFDLVIGGSPCNDLSIVNPARKGLYEGT
GRLFFEFYRLLHDARPKEGDDRPFFWLFENVVAMGVSDKRDISRF
LESNPVMIDAKEVSAAHRARYFWGNLPGMNRPLASTVNDKLELQE
CLEHGRIAKFSKVRTITTRSNSIKQGKDQHFPVFMNEKEDILWCT
EMERVFGFPVHYTDVSNMSRLARQRLLGRSWSVPVIRHLFAPLKE
YFACVSSGNSNANSRGPSFSSGLVPLSLRGSHMNPLEMFETVPVW
RRQPVRVLSLFEDIKKELTSLGFLESGSDPGQLKHVVDVTDTVRK
DVEEWGPFDLVYGATPPLGHTCDRPPSWYLFQFHRLLQYARPKPG
SPRPFFWMFVDNLVLNKEDLDVASRFLEMEPVTIPDVHGGSLQNA
VRVWSNIPAIRSRHWALVSEEELSLLAQNKQSSKLAAKWPTKLVK
NCFLPLREYFKYFSTELTSSLSGGKRPAATKKAGQAKKKKGS

In some embodiments, a site-specific disrupting agent comprises a targeting moiety (e.g., comprising dCas9, e.g., an S. aureus dCas9), and an effector moiety comprising HDAC8, e.g., a HDAC8 domain. In some embodiments, a site-specific disrupting agent comprises a targeting moiety comprising dCas9, e.g., an S. pyogenes dCas9, e.g., mutant S. pyogenes Cas9, e.g., Cas9m4, and an effector moiety comprising HDAC8, e.g., a HDAC8 domain. In some embodiments, the site-specific disrupting agent is encoded by the nucleic acid sequence of SEQ ID NO: 213 (e.g., mRNA encoding the site-specific disrupting agent).

In some embodiments, a nucleic acid described herein comprises a nucleic acid sequence of SEQ ID NO: 213 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-HDAC8 mRNA (MR-29439)

(SEQ ID NO: 213)
AGGAAAUAAGAGAGAAAAGAAGAGUAAGAAGAAAUAUAAGAGCCA
CCAUGGCCCCCAAGAAGAAGCGGAAGGUGGGCAUCCACGGCGUGC
CCGCCGCCGACAAGAAGUACAGCAUCGGCCUGGCCAUCGGCACCA
ACAGCGUGGGCUGGGCCGUGAUCACCGACGAGUACAAGGUGCCCA
GCAAGAAGUUCAAGGUGCUGGGCAACACCGACCGGCACAGCAUCA
AGAAGAACCUGAUCGGCGCCCUGCUGUUCGACAGCGGCGAGACCG
CCGAGGCCACCCGGCUGAAGCGGACCGCCCGGCGGCGGUACACCC
GGCGGAAGAACCGGAUCUGCUACCUGCAGGAGAUCUUCAGCAACG
AGAUGGCCAAGGUGGACGACAGCUUCUUCCACCGGCUGGAGGAGA
GCUUCCUGGUGGAGGAGGACAAGAAGCACGAGCGGCACCCCAUCU
UCGGCAACAUCGUGGACGAGGUGGCCUACCACGAGAAGUACCCCA
CCAUCUACCACCUGCGGAAGAAGCUGGUGGACAGCACCGACAAGG
CCGACCUGCGGCUGAUCUACCUGGCCCUGGCCCACAUGAUCAAGU
UCCGGGGCCACUUCCUGAUCGAGGGCGACCUGAACCCCGACAACA
GCGACGUGGACAAGCUGUUCAUCCAGCUGGUGCAGACCUACAACC
AGCUGUUCGAGGAGAACCCCAUCAACGCCAGCGGCGUGGACGCCA
AGGCCAUCCUGAGCGCCCGGCUGAGCAAGAGCCGGCGGCUGGAGA
ACCUGAUCGCCCAGCUGCCCGGCGAGAAGAAGAACGGCCUGUUCG
GCAACCUGAUCGCCCUGAGCCUGGGCCUGACCCCCAACUUCAAGA
GCAACUUCGACCUGGCCGAGGACGCCAAGCUGCAGCUGAGCAAGG
ACACCUACGACGACGACCUGGACAACCUGCUGGCCCAGAUCGGCG
ACCAGUACGCCGACCUGUUCCUGGCCGCCAAGAACCUGAGCGACG
CCAUCCUGCUGAGCGACAUCCUGCGGGUGAACACCGAGAUCACCA
AGGCCCCCCUGAGCGCCAGCAUGAUCAAGCGGUACGACGAGCACC
ACCAGGACCUGACCCUGCUGAAGGCCCUGGUGCGGCAGCAGCUGC
CCGAGAAGUACAAGGAGAUCUUCUUCGACCAGAGCAAGAACGGCU
ACGCCGGCUACAUCGACGGCGGCGCCAGCCAGGAGGAGUUCUACA
AGUUCAUCAAGCCCAUCCUGGAGAAGAUGGACGGCACCGAGGAGC
UGCUGGUGAAGCUGAACCGGGAGGACCUGCUGCGGAAGCAGCGGA
CCUUCGACAACGGCAGCAUCCCCCACCAGAUCCACCUGGGCGAGC
UGCACGCCAUCCUGCGGCGGCAGGAGGACUUCUACCCCUUCCUGA
AGGACAACCGGGAGAAGAUCGAGAAGAUCCUGACCUUCCGGAUCC
CCUACUACGUGGGCCCCCUGGCCCGGGGCAACAGCCGGUUCGCCU
GGAUGACCCGGAAAUCCGAGGAGACCAUCACCCCCUGGAACUUCG
AGGAGGUGGUGGACAAGGGCGCCAGCGCCCAGAGCUUCAUCGAGC
GGAUGACCAACUUCGACAAGAACCUGCCCAACGAGAAGGUGCUGC
CCAAGCACAGCCUGCUGUACGAGUACUUCACCGUGUACAACGAGC
UGACCAAGGUGAAGUACGUGACCGAGGGCAUGCGGAAGCCCGCCU
UCCUGAGCGGCGAGCAGAAGAAGGCCAUCGUGGACCUGCUGUUCA
AGACCAACCGGAAGGUGACCGUGAAGCAGCUGAAGGAGGACUACU
UCAAGAAGAUCGAGUGCUUCGACAGCGUGGAGAUCAGCGGCGUGG
AGGACCGGUUCAACGCCAGCCUGGGCACCUACCACGACCUGCUGA
AGAUCAUCAAGGACAAGGACUUCCUGGACAACGAGGAGAACGAGG
ACAUCCUGGAGGACAUCGUGCUGACCCUGACCCUGUUCGAGGACC
GGGAGAUGAUCGAGGAGCGGCUGAAAACCUACGCCCACCUGUUCG
ACGACAAGGUGAUGAAGCAGCUGAAGCGGCGGCGGUACACCGGCU
GGGGCCGGCUGAGCCGGAAGCUGAUCAACGGCAUCCGGGACAAGC
AGAGCGGCAAGACCAUCCUGGACUUCCUGAAAUCCGACGGCUUCG
CCAACCGGAACUUCAUGCAGCUGAUCCACGACGACAGCCUGACCU
UCAAGGAGGACAUCCAGAAGGCCCAGGUGAGCGGCCAGGGCGACA
GCCUGCACGAGCACAUCGCCAACCUGGCCGGCAGCCCCGCCAUCA
AGAAGGGCAUCCUGCAGACCGUGAAGGUGGUGGACGAGCUGGUGA
AGGUGAUGGGCCGGCACAAGCCCGAGAACAUCGUGAUCGAGAUGG
CCCGGGAGAACCAGACCACCCAGAAGGGCCAGAAGAACAGCCGGG
AGCGGAUGAAGCGGAUCGAGGAGGGCAUCAAGGAGCUGGGCAGCC
AGAUCCUGAAGGAGCACCCCGUGGAGAACACCCAGCUGCAGAACG
AGAAGCUGUACCUGUACUACCUGCAGAACGGCCGGGACAUGUACG
UGGACCAGGAGCUGGACAUCAACCGGCUGAGCGACUACGACGUGG
CCGCCAUCGUGCCCCAGAGCUUCCUGAAGGACGACAGCAUCGACA
ACAAGGUGCUGACCCGGAGCGACAAGGCCCGGGGCAAGAGCGACA
ACGUGCCCAGCGAGGAGGUGGUGAAGAAGAUGAAGAACUACUGGC
GGCAGCUGCUGAACGCCAAGCUGAUCACCCAGCGGAAGUUCGACA
ACCUGACCAAGGCCGAGCGGGGCGGCCUGAGCGAGCUGGACAAGG
CCGGCUUCAUCAAGCGGCAGCUGGUGGAGACCCGGCAGAUCACCA
AGCACGUGGCCCAGAUCCUGGACAGCCGGAUGAACACCAAGUACG
ACGAGAACGACAAGCUGAUCCGGGAGGUGAAGGUGAUCACCCUGA
AAUCCAAGCUGGUGAGCGACUUCCGGAAGGACUUCCAGUUCUACA
AGGUGCGGGAGAUCAACAACUACCACCACGCCCACGACGCCUACC
UGAACGCCGUGGUGGGCACCGCCCUGAUCAAGAAGUACCCCAAGC
UGGAGAGCGAGUUCGUGUACGGCGACUACAAGGUGUACGACGUGC
GGAAGAUGAUCGCCAAGAGCGAGCAGGAGAUCGGCAAGGCCACCG
CCAAGUACUUCUUCUACAGCAACAUCAUGAACUUCUUCAAGACCG
AGAUCACCCUGGCCAACGGCGAGAUCCGGAAGCGGCCCCUGAUCG
AGACCAACGGCGAGACCGGCGAGAUCGUGUGGGACAAGGGCCGGG
ACUUCGCCACCGUGCGGAAGGUGCUGAGCAUGCCCCAGGUGAACA
UCGUGAAGAAAACCGAGGUGCAGACCGGCGGCUUCAGCAAGGAGA
GCAUCCUGCCCAAGCGGAACAGCGACAAGCUGAUCGCCCGGAAGA
AGGACUGGGACCCCAAGAAGUACGGCGGCUUCGACAGCCCCACCG
UGGCCUACAGCGUGCUGGUGGUGGCCAAGGUGGAGAAGGGCAAGA
GCAAGAAGCUGAAAUCCGUGAAGGAGCUGCUGGGCAUCACCAUCA
UGGAGCGGAGCAGCUUCGAGAAGAACCCCAUCGACUUCCUGGAGG
CCAAGGGCUACAAGGAGGUGAAGAAGGACCUGAUCAUCAAGCUGC
CCAAGUACAGCCUGUUCGAGCUGGAGAACGGCCGGAAGCGGAUGC
UGGCCAGCGCCGGCGAGCUGCAGAAGGGCAACGAGCUGGCCCUGC
CCAGCAAGUACGUGAACUUCCUGUACCUGGCCAGCCACUACGAGA
AGCUGAAGGGCAGCCCCGAGGACAACGAGCAGAAGCAGCUGUUCG
UGGAGCAGCACAAGCACUACCUGGACGAGAUCAUCGAGCAGAUCA
GCGAGUUCAGCAAGCGGGUGAUCCUGGCCGACGCCAACCUGGACA
AGGUGCUGAGCGCCUACAACAAGCACCGGGACAAGCCCAUCCGGG
AGCAGGCCGAGAACAUCAUCCACCUGUUCACCCUGACCAACCUGG
GCGCCCCCGCCGCCUUCAAGUACUUCGACACCACCAUCGACCGGA
AGCGGUACACCAGCACCAAGGAGGUGCUGGACGCCACCCUGAUCC
ACCAGAGCAUCACCGGCCUGUACGAGACCCGGAUCGACCUGAGCC
AGCUGGGCGGCGACAGCGGCGGCAAGCGGCCCGCCGCCACCAAGA
AGGCCGGCCAGGCCAAGAAGAAGAAGUCGGGCGGGGGUGGCUCAG
AGGAGCCCGAGGAGCCCGCCGAUAGCGGACAAUCUCUGGUGCCCG
UCUACAUCUACAGCCCCGAAUAUGUGAGCAUGUGUGAUUCCCUCG
CCAAGAUCCCUAAGAGAGCCAGCAUGGUGCAUUCUCUGAUCGAGG
CCUACGCUCUGCAUAAGCAAAUGAGGAUCGUGAAGCCCAAGGUCG
CCAGCAUGGAAGAGAUGGCCACCUUUCACACCGAUGCCUACCUCC
AACAUCUCCAGAAGGUGUCCCAAGAGGGCGACGACGACCACCCCG
ACUCCAUUGAGUACGGACUGGGCUAUGAUUGCCCCGCCACCGAGG
GCAUCUUUGACUAUGCCGCCGCUAUCGGCGGAGCUACCAUCACAG
CCGCCCAGUGUCUGAUUGAUGGCAUGUGCAAGGUCGCCAUCAACU
GGUCCGGAGGCUGGCAUCAUGCCAAGAAGGAUGAGGCCUCCGGCU
UCUGUUAUCUGAAUGACGCCGUGCUGGGCAUUCUGAGACUGAGGA
GGAAAUUCGAGAGGAUUCUGUACGUGGAUCUGGAUCUGCAUCACG
GAGAUGGAGUCGAAGAUGCCUUCAGCUUCACCAGCAAGGUGAUGA
CAGUCUCUCUGCACAAGUUCUCCCCCGGCUUCUUUCCCGGAACCG
GCGACGUGUCCGACGUGGGACUGGGCAAGGGAAGGUACUACAGCG
UGAACGUGCCCAUUCAAGACGGCAUCCAAGACGAGAAGUACUACC
AGAUCUGCGAGUCCGUGCUCAAGGAGGUCUACCAAGCCUUCAAUC
CUAAGGCUGUCGUGCUCCAACUGGGAGCUGAUACCAUUGCUGGCG
AUCCCAUGUGCAGCUUCAAUAUGACACCCGUCGGAAUCGGCAAGU
GCCUCAAGUACAUCCUCCAGUGGCAGCUCGCCACCCUCAUUCUCG
GAGGAGGCGGAUACAAUCUGGCUAAUACCGCCAGAUGCUGGACCU
AUCUGACCGGCGUGAUUCUGGGCAAAACACUGAGCAGCGAAAUCC
CCGACCACGAGUUUUUCACCGCUUACGGCCCCGACUACGUGCUGG
AGAUCACCCCCAGCUGCAGACCCGAUAGAAACGAACCCCAUAGAA
UCCAGCAAAUUCUGAACUAUAUCAAGGGCAACCUCAAGCACGUCG
UGGGAGGUGGCGGAUCGGGAAAGCGGCCCGCCGCCACCAAGAAGG
CCGGUCAGGCCAAGAAGAAGAAGGGCAGCUACCCCUACGACGUGC
CCGACUACGCCUGAGCGGCCGCUUAAUUAAGCUGCCUUCUGCGGG
GCUUGCCUUCUGGCCAUGCCCUUCUUCUCUCCCUUGCACCUGUAC
CUCUUGGUCUUUGAAUAAAGCCUGAGUAGGAAGUCUAGAAAAAAA
AAAAAAAAAAAAAAAAAAAAAAAAAAAAAAAAAAAAAAAAAAAAA
AAAAAAAAAAAAAAAAAAAAAAAAAAAA

In some embodiments, a site-specific disrupting agent comprises the amino acid sequence of SEQ ID NOs: 214 or 78. In some embodiments, a site-specific disrupting agent described herein comprises an amino acid sequence of SEQ ID NO: 214 or 78 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-HDAC8 Protein Sequence (Corresponding to MR 29439)

(SEQ ID NO: 214)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDSGGKRPAATKK
AGQAKKKKSGGGGSEEPEEPADSGQSLVPVYIYSPEYVSMCDSLA
KIPKRASMVHSLIEAYALHKQMRIVKPKVASMEEMATFHTDAYLQ
HLQKVSQEGDDDHPDSIEYGLGYDCPATEGIFDYAAAIGGATITA
AQCLIDGMCKVAINWSGGWHHAKKDEASGFCYLNDAVLGILRLRR
KFERILYVDLDLHHGDGVEDAFSFTSKVMTVSLHKFSPGFFPGTG
DVSDVGLGKGRYYSVNVPIQDGIQDEKYYQICESVLKEVYQAFNP
KAVVLQLGADTIAGDPMCSFNMTPVGIGKCLKYILQWQLATLILG
GGGYNLANTARCWTYLTGVILGKTLSSEIPDHEFFTAYGPDYVLE
ITPSCRPDRNEPHRIQQILNYIKGNLKHVVGGGGSGKRPAATKKA
GQAKKKKGSYPYDVPDYA

dCas9-HDAC8 without HA Tag

(SEQ ID NO: 78)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDSGGKRPAATKK
AGQAKKKKSGGGGSEEPEEPADSGQSLVPVYIYSPEYVSMCDSLA
KIPKRASMVHSLIEAYALHKQMRIVKPKVASMEEMATFHTDAYLQ
HLQKVSQEGDDDHPDSIEYGLGYDCPATEGIFDYAAAIGGATITA
AQCLIDGMCKVAINWSGGWHHAKKDEASGFCYLNDAVLGILRLRR
KFERILYVDLDLHHGDGVEDAFSFTSKVMTVSLHKFSPGFFPGTG
DVSDVGLGKGRYYSVNVPIQDGIQDEKYYQICESVLKEVYQAFNP
KAVVLQLGADTIAGDPMCSFNMTPVGIGKCLKYILQWQLATLILG
GGGYNLANTARCWTYLTGVILGKTLSSEIPDHEFFTAYGPDYVLE
ITPSCRPDRNEPHRIQQILNYIKGNLKHVVGGGGSGKRPAATKKA
GQAKKKKGS

In some embodiments, a site-specific disrupting agent comprises a targeting moiety (e.g., comprising dCas9, e.g., an S. aureus dCas9), a first effector moiety comprising EZH2; e.g., an EZH2 domain, and a second effector moiety comprising HDAC8, e.g., an HDAC8 domain. In some embodiments, a site-specific disrupting agent comprises a targeting moiety comprising dCas9, e.g., an S. pyogenes dCas9, e.g., a mutant S. pyogenes Cas9, e.g., Cas9m4; a first effector moiety comprising EZH2, e.g., an EZH2 domain; and a second effector moiety comprising HDAC8, e.g., an HDAC8 domain. In some embodiments, the site-specific disrupting agent is encoded by the nucleic acid sequence of SEQ ID NO: 215 (e.g., mRNA encoding the site-specific disrupting agent). In some embodiments, a nucleic acid described herein comprises a nucleic acid sequence of SEQ ID NO: 215 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2-dCas9-HDAC8 mRNA MR-29447

(SEQ ID NO: 215)
AGGAAAUAAGAGAGAAAAGAAGAGUAAGAAGAAAUAUAAGAGCCA
CCAUGGCCCCCAAGAAGAAGCGGAAGGUGGGCGGCAGCGGCGGCA
GCGGCCAGACCGGCAAGAAGAGCGAGAAGGGCCCCGUGUGCUGGC
GGAAGCGGGUGAAGAGCGAGUACAUGCGGCUGCGGCAGCUGAAGC
GGUUCCGGCGGGCCGACGAGGUGAAGAGCAUGUUCAGCAGCAACC
GGCAGAAGAUCCUGGAGCGGACCGAGAUCCUGAACCAGGAGUGGA
AGCAGCGGCGAAUCCAGCCCGUGCACAUCCUGACCAGCGUGAGCA
GCCUGCGGGGCACCCGGGAGUGCAGCGUGACCAGCGACCUGGACU
UCCCCACCCAGGUGAUCCCCCUAAAGACCCUGAACGCCGUGGCCA
GCGUGCCCAUCAUGUACAGCUGGAGCCCCCUGCAGCAGAACUUCA
UGGUGGAGGACGAGACCGUGCUGCACAACAUCCCCUACAUGGGCG
ACGAGGUGCUGGACCAGGACGGCACCUUCAUCGAGGAGCUGAUCA
AGAACUACGACGGCAAGGUGCACGGCGACCGGGAGUGCGGCUUCA
UCAACGACGAGAUCUUCGUGGAGCUGGUGAACGCCCUGGGCCAGU
ACAACGACGACGACGACGACGACGACGGCGACGACCCCGAGGAGC
GGGAGGAGAAGCAGAAGGACCUGGAGGACCACCGGGACGACAAGG
AGAGCCGGCCCCCCCGGAAGUUCCCCAGCGACAAGAUCUUCGAGG
CCAUCAGCAGCAUGUUCCCCGACAAGGGCACCGCCGAGGAGCUGA
AGGAGAAGUACAAGGAGCUGACCGAGCAGCAGCUGCCCGGCGCCC
UGCCCCCCGAGUGCACCCCCAACAUCGACGGCCCCAACGCCAAGA
GCGUGCAGCGGGAGCAGAGCCUGCACAGCUUCCACACCCUGUUCU
GCCGGCGGUGCUUCAAGUACGACUGCUUCCUGCACCCCUUCCACG
CCACCCCCAACACCUACAAGCGGAAGAACACCGAGACCGCCCUGG
ACAACAAGCCCUGCGGCCCCCAGUGCUACCAGCACCUGGAGGGCG
CCAAGGAGUUCGCCGCCGCCCUGACCGCCGAGCGGAUCAAGACCC
CCCCCAAGCGGCCCGGCGGCCGGCGGCGGGGCCGGCUGCCCAACA
ACAGCAGCCGGCCCAGCACCCCCACCAUCAACGUGCUGGAGAGCA
AGGACACCGACAGCGACCGGGAGGCCGGCACCGAGACCGGCGGCG
AGAACAACGACAAGGAGGAGGAGGAGAAGAAGGACGAGACCAGCA
GCAGCAGCGAGGCCAACAGCCGGUGCCAGACCCCCAUCAAGAUGA
AGCCCAACAUCGAGCCCCCCGAGAACGUGGAGUGGAGCGGCGCCG
AGGCCAGCAUGUUCCGGGUGCUGAUCGGCACCUACUACGACAACU
UCUGCGCCAUCGCCCGGCUGAUCGGCACCAAGACCUGCCGGCAGG
UGUACGAGUUCCGGGUGAAGGAGAGCAGCAUCAUCGCCCCCGCCC
CCGCCGAGGACGUGGACACCCCCCCCCGGAAGAAGAAGCGGAAGC
ACCGGCUGUGGGCCGCCCACUGCCGGAAGAUCCAGCUGAAGAAGG
ACGGCAGCAGCAACCACGUGUACAACUACCAGCCCUGCGACCACC
CCCGGCAGCCCUGCGACAGCAGCUGCCCCUGCGUGAUCGCCCAGA
ACUUCUGCGAGAAGUUCUGCCAGUGCAGCAGCGAGUGCCAGAACC
GGUUCCCCGGCUGCCGGUGCAAGGCCCAGUGCAACACCAAGCAGU
GCCCCUGCUACCUGGCCGUGCGGGAGUGCGACCCCGACCUGUGCC
UGACCUGCGGCGCCGCCGACCACUGGGACAGCAAGAACGUGAGCU
GCAAGAACUGCAGCAUCCAGCGGGGCAGCAAGAAGCACCUGCUGC
UGGCCCCCAGCGACGUGGCCGGCUGGGGCAUCUUCAUCAAGGACC
CCGUGCAGAAGAACGAGUUCAUCAGCGAGUACUGCGGCGAGAUCA
UCAGCCAGGACGAGGCCGACCGGCGGGGCAAGGUGUACGACAAGU
ACAUGUGCAGCUUCCUGUUCAACCUGAACAACGACUUCGUGGUGG
ACGCCACCCGGAAGGGCAACAAGAUCCGGUUCGCCAACCACAGCG
UGAACCCCAACUGCUACGCCAAGGUGAUGAUGGUGAACGGCGACC
ACCGGAUCGGCAUCUUCGCCAAGCGGGCCAUCCAGACCGGCGAGG
AGCUGUUCUUCGACUACCGGUACAGCCAGGCCGACGCCCUGAAGU
ACGUGGGCAUCGAGCGGGAGAUGGAGAUCCCCAGCACCGGCGGCA
GCGGCGGCAGCGGCGGCAGCGGCGGCAGCGGCGGCAGCGGCCGAC
CCGACAAGAAGUACAGCAUCGGCCUGGCCAUCGGCACCAACAGCG
UGGGCUGGGCCGUGAUCACCGACGAGUACAAGGUGCCCAGCAAGA
AGUUCAAGGUGCUGGGCAACACCGACCGGCACAGCAUCAAGAAGA
ACCUGAUCGGCGCCCUGCUGUUCGACAGCGGCGAGACCGCCGAGG
CCACCCGGCUGAAGCGGACCGCCCGGCGGCGGUACACCCGGCGGA
AGAACCGGAUCUGCUACCUGCAGGAGAUCUUCAGCAACGAGAUGG
CCAAGGUGGACGACAGCUUCUUCCACCGGCUGGAGGAGAGCUUCC
UGGUGGAGGAGGACAAGAAGCACGAGCGGCACCCCAUCUUCGGCA
ACAUCGUGGACGAGGUGGCCUACCACGAGAAGUACCCCACCAUCU
ACCACCUGCGGAAGAAGCUGGUGGACAGCACCGACAAGGCCGACC
UGCGGCUGAUCUACCUGGCCCUGGCCCACAUGAUCAAGUUCCGGG
GCCACUUCCUGAUCGAGGGCGACCUGAACCCCGACAACAGCGACG
UGGACAAGCUGUUCAUCCAGCUGGUGCAGACCUACAACCAGCUGU
UCGAGGAGAACCCCAUCAACGCCAGCGGCGUGGACGCCAAGGCCA
UCCUGAGCGCCCGGCUGAGCAAGAGCCGGCGGCUGGAGAACCUGA
UCGCCCAGCUGCCCGGCGAGAAGAAGAACGGCCUGUUCGGCAACC
UGAUCGCCCUGAGCCUGGGCCUGACCCCCAACUUCAAGAGCAACU
UCGACCUGGCCGAGGACGCCAAGCUGCAGCUGAGCAAGGACACCU
ACGACGACGACCUGGACAACCUGCUGGCCCAGAUCGGCGACCAGU
ACGCCGACCUGUUCCUGGCCGCCAAGAACCUGAGCGACGCCAUCC
UGCUGAGCGACAUCCUGCGGGUGAACACCGAGAUCACCAAGGCCC
CCCUGAGCGCCAGCAUGAUCAAGCGGUACGACGAGCACCACCAGG
ACCUGACCCUGCUGAAGGCCCUGGUGCGGCAGCAGCUGCCCGAGA
AGUACAAGGAGAUCUUCUUCGACCAGAGCAAGAACGGCUACGCCG
GCUACAUCGACGGCGGCGCCAGCCAGGAGGAGUUCUACAAGUUCA
UCAAGCCCAUCCUGGAGAAGAUGGACGGCACCGAGGAGCUGCUGG
UGAAGCUGAACCGGGAGGACCUGCUGCGGAAGCAGCGGACCUUCG
ACAACGGCAGCAUCCCCCACCAGAUCCACCUGGGCGAGCUGCACG
CCAUCCUGCGGCGGCAGGAGGACUUCUACCCCUUCCUGAAGGACA
ACCGGGAGAAGAUCGAGAAGAUCCUGACCUUCCGGAUCCCCUACU
ACGUGGGCCCCCUGGCCCGGGGCAACAGCCGGUUCGCCUGGAUGA
CCCGGAAAUCCGAGGAGACCAUCACCCCCUGGAACUUCGAGGAGG
UGGUGGACAAGGGCGCCAGCGCCCAGAGCUUCAUCGAGCGGAUGA
CCAACUUCGACAAGAACCUGCCCAACGAGAAGGUGCUGCCCAAGC
ACAGCCUGCUGUACGAGUACUUCACCGUGUACAACGAGCUGACCA
AGGUGAAGUACGUGACCGAGGGCAUGCGGAAGCCCGCCUUCCUGA
GCGGCGAGCAGAAGAAGGCCAUCGUGGACCUGCUGUUCAAGACCA
ACCGGAAGGUGACCGUGAAGCAGCUGAAGGAGGACUACUUCAAGA
AGAUCGAGUGCUUCGACAGCGUGGAGAUCAGCGGCGUGGAGGACC
GGUUCAACGCCAGCCUGGGCACCUACCACGACCUGCUGAAGAUCA
UCAAGGACAAGGACUUCCUGGACAACGAGGAGAACGAGGACAUCC
UGGAGGACAUCGUGCUGACCCUGACCCUGUUCGAGGACCGGGAGA
UGAUCGAGGAGCGGCUGAAAACCUACGCCCACCUGUUCGACGACA
AGGUGAUGAAGCAGCUGAAGCGGCGGCGGUACACCGGCUGGGGCC
GGCUGAGCCGGAAGCUGAUCAACGGCAUCCGGGACAAGCAGAGCG
GCAAGACCAUCCUGGACUUCCUGAAAUCCGACGGCUUCGCCAACC
GGAACUUCAUGCAGCUGAUCCACGACGACAGCCUGACCUUCAAGG
AGGACAUCCAGAAGGCCCAGGUGAGCGGCCAGGGCGACAGCCUGC
ACGAGCACAUCGCCAACCUGGCCGGCAGCCCCGCCAUCAAGAAGG
GCAUCCUGCAGACCGUGAAGGUGGUGGACGAGCUGGUGAAGGUGA
UGGGCCGGCACAAGCCCGAGAACAUCGUGAUCGAGAUGGCCCGGG
AGAACCAGACCACCCAGAAGGGCCAGAAGAACAGCCGGGAGCGGA
UGAAGCGGAUCGAGGAGGGCAUCAAGGAGCUGGGCAGCCAGAUCC
UGAAGGAGCACCCCGUGGAGAACACCCAGCUGCAGAACGAGAAGC
UGUACCUGUACUACCUGCAGAACGGCCGGGACAUGUACGUGGACC
AGGAGCUGGACAUCAACCGGCUGAGCGACUACGACGUGGCCGCCA
UCGUGCCCCAGAGCUUCCUGAAGGACGACAGCAUCGACAACAAGG
UGCUGACCCGGAGCGACAAGGCCCGGGGCAAGAGCGACAACGUGC
CCAGCGAGGAGGUGGUGAAGAAGAUGAAGAACUACUGGCGGCAGC
UGCUGAACGCCAAGCUGAUCACCCAGCGGAAGUUCGACAACCUGA
CCAAGGCCGAGCGGGGCGGCCUGAGCGAGCUGGACAAGGCCGGCU
UCAUCAAGCGGCAGCUGGUGGAGACCCGGCAGAUCACCAAGCACG
UGGCCCAGAUCCUGGACAGCCGGAUGAACACCAAGUACGACGAGA
ACGACAAGCUGAUCCGGGAGGUGAAGGUGAUCACCCUGAAAUCCA
AGCUGGUGAGCGACUUCCGGAAGGACUUCCAGUUCUACAAGGUGC
GGGAGAUCAACAACUACCACCACGCCCACGACGCCUACCUGAACG
CCGUGGUGGGCACCGCCCUGAUCAAGAAGUACCCCAAGCUGGAGA
GCGAGUUCGUGUACGGCGACUACAAGGUGUACGACGUGCGGAAGA
UGAUCGCCAAGAGCGAGCAGGAGAUCGGCAAGGCCACCGCCAAGU
ACUUCUUCUACAGCAACAUCAUGAACUUCUUCAAGACCGAGAUCA
CCCUGGCCAACGGCGAGAUCCGGAAGCGGCCCCUGAUCGAGACCA
ACGGCGAGACCGGCGAGAUCGUGUGGGACAAGGGCCGGGACUUCG
CCACCGUGCGGAAGGUGCUGAGCAUGCCCCAGGUGAACAUCGUGA
AGAAAACCGAGGUGCAGACCGGCGGCUUCAGCAAGGAGAGCAUCC
UGCCCAAGCGGAACAGCGACAAGCUGAUCGCCCGGAAGAAGGACU
GGGACCCCAAGAAGUACGGCGGCUUCGACAGCCCCACCGUGGCCU
ACAGCGUGCUGGUGGUGGCCAAGGUGGAGAAGGGCAAGAGCAAGA
AGCUGAAAUCCGUGAAGGAGCUGCUGGGCAUCACCAUCAUGGAGC
GGAGCAGCUUCGAGAAGAACCCCAUCGACUUCCUGGAGGCCAAGG
GCUACAAGGAGGUGAAGAAGGACCUGAUCAUCAAGCUGCCCAAGU
ACAGCCUGUUCGAGCUGGAGAACGGCCGGAAGCGGAUGCUGGCCA
GCGCCGGCGAGCUGCAGAAGGGCAACGAGCUGGCCCUGCCCAGCA
AGUACGUGAACUUCCUGUACCUGGCCAGCCACUACGAGAAGCUGA
AGGGCAGCCCCGAGGACAACGAGCAGAAGCAGCUGUUCGUGGAGC
AGCACAAGCACUACCUGGACGAGAUCAUCGAGCAGAUCAGCGAGU
UCAGCAAGCGGGUGAUCCUGGCCGACGCCAACCUGGACAAGGUGC
UGAGCGCCUACAACAAGCACCGGGACAAGCCCAUCCGGGAGCAGG
CCGAGAACAUCAUCCACCUGUUCACCCUGACCAACCUGGGCGCCC
CCGCCGCCUUCAAGUACUUCGACACCACCAUCGACCGGAAGCGGU
ACACCAGCACCAAGGAGGUGCUGGACGCCACCCUGAUCCACCAGA
GCAUCACCGGCCUGUACGAGACCCGGAUCGACCUGAGCCAGCUGG
GCGGCGACAGCGGCGGCAAGCGGCCCGCCGCCACCAAGAAGGCCG
GCCAGGCCAAGAAGAAGAAGUCGGGCGGGGGUGGCUCAGAGGAGC
CCGAGGAGCCCGCCGAUAGCGGACAAUCUCUGGUGCCCGUCUACA
UCUACAGCCCCGAAUAUGUGAGCAUGUGUGAUUCCCUCGCCAAGA
UCCCUAAGAGAGCCAGCAUGGUGCAUUCUCUGAUCGAGGCCUACG
CUCUGCAUAAGCAAAUGAGGAUCGUGAAGCCCAAGGUCGCCAGCA
UGGAAGAGAUGGCCACCUUUCACACCGAUGCCUACCUCCAACAUC
UCCAGAAGGUGUCCCAAGAGGGCGACGACGACCACCCCGACUCCA
UUGAGUACGGACUGGGCUAUGAUUGCCCCGCCACCGAGGGCAUCU
UUGACUAUGCCGCCGCUAUCGGCGGAGCUACCAUCACAGCCGCCC
AGUGUCUGAUUGAUGGCAUGUGCAAGGUCGCCAUCAACUGGUCCG
GAGGCUGGCAUCAUGCCAAGAAGGAUGAGGCCUCCGGCUUCUGUU
AUCUGAAUGACGCCGUGCUGGGCAUUCUGAGACUGAGGAGGAAAU
UCGAGAGGAUUCUGUACGUGGAUCUGGAUCUGCAUCACGGAGAUG
GAGUCGAAGAUGCCUUCAGCUUCACCAGCAAGGUGAUGACAGUCU
CUCUGCACAAGUUCUCCCCCGGCUUCUUUCCCGGAACCGGCGACG
UGUCCGACGUGGGACUGGGCAAGGGAAGGUACUACAGCGUGAACG
UGCCCAUUCAAGACGGCAUCCAAGACGAGAAGUACUACCAGAUCU
GCGAGUCCGUGCUCAAGGAGGUCUACCAAGCCUUCAAUCCUAAGG
CUGUCGUGCUCCAACUGGGAGCUGAUACCAUUGCUGGCGAUCCCA
UGUGCAGCUUCAAUAUGACACCCGUCGGAAUCGGCAAGUGCCUCA
AGUACAUCCUCCAGUGGCAGCUCGCCACCCUCAUUCUCGGAGGAG
GCGGAUACAAUCUGGCUAAUACCGCCAGAUGCUGGACCUAUCUGA
CCGGCGUGAUUCUGGGCAAAACACUGAGCAGCGAAAUCCCCGACC
ACGAGUUUUUCACCGCUUACGGCCCCGACUACGUGCUGGAGAUCA
CCCCCAGCUGCAGACCCGAUAGAAACGAACCCCAUAGAAUCCAGC
AAAUUCUGAACUAUAUCAAGGGCAACCUCAAGCACGUCGUGGGAG
GUGGCGGAUCGGGAAAGCGGCCCGCCGCCACCAAGAAGGCCGGUC
AGGCCAAGAAGAAGAAGGGCAGCUACCCCUACGACGUGCCCGACU
ACGCCUGAGCGGCCGCUUAAUUAAGCUGCCUUCUGCGGGGCUUGC
CUUCUGGCCAUGCCCUUCUUCUCUCCCUUGCACCUGUACCUCUUG
GUCUUUGAAUAAAGCCUGAGUAGGAAGUCUAGAAAAAAAAAAAAA
AAAAAAAAAAAAAAAAAAAAAAAAAAAAAAAAAAAAAAAAAAAAA
AAAAAAAAAAAAAAAAAAAAAA

In some embodiments, a site-specific disrupting agent comprises the amino acid sequence of SEQ ID NOs: 216 or 79. In some embodiments, a site-specific disrupting agent described herein comprises an amino acid sequence of SEQ ID NO: 216 or 79 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### EZH2-dCas9-HDAC8 Protein Sequence (Corresponding to MR-29447)

(SEQ ID NO: 216)
MAPKKKRKVGGSGGSGQTGKKSEKGPVCWRKRVKSEYMRLRQLKR
FRRADEVKSMFSSNRQKILERTEILNQEWKQRRIQPVHILTSVSS
LRGTRECSVTSDLDFPTQVIPLKTLNAVASVPIMYSWSPLQQNFM
VEDETVLHNIPYMGDEVLDQDGTFIEELIKNYDGKVHGDRECGFI
NDEIFVELVNALGQYNDDDDDDDGDDPEEREEKQKDLEDHRDDKE
SRPPRKFPSDKIFEAISSMFPDKGTAEELKEKYKELTEQQLPGAL
PPECTPNIDGPNAKSVQREQSLHSFHTLFCRRCFKYDCFLHPFHA
TPNTYKRKNTETALDNKPCGPQCYQHLEGAKEFAAALTAERIKTP
PKRPGGRRRGRLPNNSSRPSTPTINVLESKDTDSDREAGTETGGE
NNDKEEEEKKDETSSSSEANSRCQTPIKMKPNIEPPENVEWSGAE
ASMFRVLIGTYYDNFCAIARLIGTKTCRQVYEFRVKESSIIAPAP
AEDVDTPPRKKKRKHRLWAAHCRKIQLKKDGSSNHVYNYQPCDHP
RQPCDSSCPCVIAQNFCEKFCQCSSECQNRFPGCRCKAQCNTKQC
PCYLAVRECDPDLCLTCGAADHWDSKNVSCKNCSIQRGSKKHLLL
APSDVAGWGIFIKDPVQKNEFISEYCGEIISQDEADRRGKVYDKY
MCSFLFNLNNDFVVDATRKGNKIRFANHSVNPNCYAKVMMVNGDH
RIGIFAKRAIQTGEELFFDYRYSQADALKYVGIEREMEIPSTGGS
GGSGGSGGSGGSGRPDKKYSIGLAIGTNSVGWAVITDEYKVPSKK
FKVLGNTDRHSIKKNLIGALLFDSGETAEATRLKRTARRRYTRRK
NRICYLQEIFSNEMAKVDDSFFHRLEESFLVEEDKKHERHPIFGN
IVDEVAYHEKYPTIYHLRKKLVDSTDKADLRLIYLALAHMIKFRG
HFLIEGDLNPDNSDVDKLFIQLVQTYNQLFEENPINASGVDAKAI
LSARLSKSRRLENLIAQLPGEKKNGLFGNLIALSLGLTPNFKSNF
DLAEDAKLQLSKDTYDDDLDNLLAQIGDQYADLFLAAKNLSDAIL
LSDILRVNTEITKAPLSASMIKRYDEHHQDLTLLKALVRQQLPEK
YKEIFFDQSKNGYAGYIDGGASQEEFYKFIKPILEKMDGTEELLV
KLNREDLLRKQRTFDNGSIPHQIHLGELHAILRRQEDFYPFLKDN
REKIEKILTFRIPYYVGPLARGNSRFAWMTRKSEETITPWNFEEV
VDKGASAQSFIERMTNFDKNLPNEKVLPKHSLLYEYFTVYNELTK
VKYVTEGMRKPAFLSGEQKKAIVDLLFKTNRKVTVKQLKEDYFKK
IECFDSVEISGVEDRFNASLGTYHDLLKIIKDKDFLDNEENEDIL
EDIVLTLTLFEDREMIEERLKTYAHLFDDKVMKQLKRRRYTGWGR
LSRKLINGIRDKQSGKTILDFLKSDGFANRNFMQLIHDDSLTFKE
DIQKAQVSGQGDSLHEHIANLAGSPAIKKGILQTVKVVDELVKVM
GRHKPENIVIEMARENQTTQKGQKNSRERMKRIEEGIKELGSQIL
KEHPVENTQLQNEKLYLYYLQNGRDMYVDQELDINRLSDYDVAAI
VPQSFLKDDSIDNKVLTRSDKARGKSDNVPSEEVVKKMKNYWRQL
LNAKLITQRKFDNLTKAERGGLSELDKAGFIKRQLVETRQITKHV
AQILDSRMNTKYDENDKLIREVKVITLKSKLVSDFRKDFQFYKVR
EINNYHHAHDAYLNAVVGTALIKKYPKLESEFVYGDYKVYDVRKM
IAKSEQEIGKATAKYFFYSNIMNFFKTEITLANGEIRKRPLIETN
GETGEIVWDKGRDFATVRKVLSMPQVNIVKKTEVQTGGFSKESIL
PKRNSDKLIARKKDWDPKKYGGFDSPTVAYSVLVVAKVEKGKSKK
LKSVKELLGITIMERSSFEKNPIDFLEAKGYKEVKKDLIIKLPKY
SLFELENGRKRMLASAGELQKGNELALPSKYVNFLYLASHYEKLK
GSPEDNEQKQLFVEQHKHYLDEIIEQISEFSKRVILADANLDKVL
SAYNKHRDKPIREQAENIIHLFTLTNLGAPAAFKYFDTTIDRKRY
TSTKEVLDATLIHQSITGLYETRIDLSQLGGDSGGKRPAATKKAG
QAKKKKSGGGGSEEPEEPADSGQSLVPVYIYSPEYVSMCDSLAKI
PKRASMVHSLIEAYALHKQMRIVKPKVASMEEMATFHTDAYLQHL
QKVSQEGDDDHPDSIEYGLGYDCPATEGIFDYAAAIGGATITAAQ
CLIDGMCKVAINWSGGWHHAKKDEASGFCYLNDAVLGILRLRRKF
ERILYVDLDLHHGDGVEDAFSFTSKVMTVSLHKFSPGFFPGTGDV
SDVGLGKGRYYSVNVPIQDGIQDEKYYQICESVLKEVYQAFNPKA
VVLQLGADTIAGDPMCSFNMTPVGIGKCLKYILQWQLATLILGGG
GYNLANTARCWTYLTGVILGKTLSSEIPDHEFFTAYGPDYVLEIT
PSCRPDRNEPHRIQQILNYIKGNLKHVVGGGGSGKRPAATKKAGQ
AKKKKGSYPYDVPDYA

EZH2-dCas9-HDAC8 without HA Tag

(SEQ ID NO: 79)
MAPKKKRKVGGSGGSGQTGKKSEKGPVCWRKRVKSEYMRLRQLKRFRRADEVKSMFSSN
RQKILERTEILNQEWKQRRIQPVHILTSVSSLRGTRECSVTSDLDFPTQVIPLKTLNAVASVPIM
YSWSPLQQNFMVEDETVLHNIPYMGDEVLDQDGTFIEELIKNYDGKVHGDRECGFINDEIFVELV
NALGQYNDDDDDDDGDDPEEREEKQKDLEDHRDDKESRPPRKFPSDKIFEAISSMFPDKGTAEE
LKEKYKELTEQQLPGALPPECTPNIDGPNAKSVQREQSLHSFHTLFCRRCFKYDCFLHPFHATPNT
YKRKNTETALDNKPCGPQCYQHLEGAKEFAAALTAERIKTPPKRPGGRRRGRLPNNSSRPSTPTI
NVLESKDTDSDREAGTETGGENNDKEEEEKKDETSSSSEANSRCQTPIKMKPNIEPPENVEWSGA
EASMFRVLIGTYYDNFCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCSSECQNRFPGCRCKA
QCNTKQCPCYLAVRECDPDLCLTCGAADHWDSKNVSCKNCSIQRGSKKHLLLAPSDVAGWGIFI
KDPVQKNEFISEYCGEIISQDEADRRGKVYDKYMCSFLFNLNNDFVVDATRKGNKIRFANHSVN
PNCYAKVMMVNGDHRIGIFAKRAIQTGEELFFDYRYSQADALKYVGIEREMEIPSTGGSGGSGG
SGGSGGSGRPDKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFIQ
LVQTYNQLFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEITKAPLS
ASMIKRYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSLL
YEYFTVYNELTKVKYVTEGMRKPAFLSGEQKKAIVDLLFKTNRKVTVKQLKEDYFKKIECFDSVE
ISGVEDRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARENQTTQKG
QKNSRERMKRIEEGIKELGSQILKEHPVENTQLQNEKLYLYYLQNGRDMYVDQELDINRLSDYD
VAAIVPQSFLKDDSIDNKVLTRSDKARGKSDNVPSEEVVKKMKNYWRQLLNAKLITQRKFDNLT
KAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVLSMPQVNI
VKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKG
NELALPSKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQSITGL
YETRIDLSQLGGDSGGKRPAATKKAGQAKKKKSGGGGSEEPEEPADSGQSLVPVYIYSPEYVSMC
DSLAKIPKRASMVHSLIEAYALHKQMRIVKPKVASMEEMATFHTDAYLQHLQKVSQEGDDDHP
DSIEYGLGYDCPATEGIFDYAAAIGGATITAAQCLIDGMCKVAINWSGGWHHAKKDEASGFCYL
NDAVLGILRLRRKFERILYVDLDLHHGDGVEDAFSFTSKVMTVSLHKFSPGFFPGTGDVSDVGLG
KGRYYSVNVPIQDGIQDEKYYQICESVLKEVYQAFNPKAVVLQLGADTIAGDPMCSFNMTPVGI
GKCLKYILQWQLATLILGGGGYNLANTARCWTYLTGVILGKTLSSEIPDHEFFTAYGPDYVLEIT
PSCRPDRNEPHRIQQILNYIKGNLKHVVGGGGSGKRPAATKKAGQAKKKKGS

In some embodiments, a site-specific disrupting agent comprises a targeting moiety (e.g., comprising dCas9, e.g., an S aureus dCas9), a first effector moiety comprising G9A; e.g., a G9A domain, and a second effector moiety comprising EZH2, e.g., an EZH2 domain. In some embodiments, a site-specific disrupting agent comprises a targeting moiety comprising dCas9, e.g., an S. pyogenes dCas9, e.g., a mutant S. pyogenes Cas9, e.g., Cas9m4; a first effector moiety comprising G9A, e.g., a G9A domain; and a second effector moiety comprising EZH2, e.g., a EZH2 domain. In some embodiments, the site-specific disrupting agent is encoded by the nucleic acid sequence of SEQ ID NO: 69 (e.g., mRNA encoding the site-specific disrupting agent). In some embodiments, a nucleic acid described herein comprises a nucleic acid sequence of SEQ ID NO: 69 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9A-dCas9-EZH2 (MR-29441) mRNA Sequence

(SEQ ID NO. 69)
AGGAAATAAGAGAGAAAAGAAGAGTAAGAAGAAATATAAGAGCCACCATGGCCCC
CAAGAAGAAGCGGAAGGTGGGCATCCACGGCGTGCCCGCCGCCTCGGGCGGGGGTGGCTCA
GGAAGCGCCGCTATCGCCGAAGTGCTGCTGAATGCCAGATGCGATCTGCATGCCGTGAACTA
CCACGGCGACACCCCTCTGCATATCGCCGCTAGAGAGAGCTACCATGACTGTGTGCTGCTGT
TTCTGAGCAGAGGCGCCAATCCCGAGCTCAGAAACAAAGAGGGCGACACCGCTTGGGATCT
GACACCCGAGAGATCCGACGTGTGGTTCGCTCTGCAACTGAATAGAAAACTGAGACTGGGC
GTCGGCAATAGGGCCATTAGAACCGAGAAGATCATCTGTAGGGACGTGGCTAGGGGCTACG
AGAACGTGCCCATCCCTTGTGTGAACGGAGTGGATGGAGAGCCTTGCCCCGAGGATTACAA
ATACATCAGCGAGAACTGCGAAACCTCCACCATGAATATCGATAGAAACATTACACACCTCC
AGCACTGTACATGCGTGGACGATTGCAGCAGCAGCAACTGTCTGTGCGGCCAACTGAGCATC
AGATGCTGGTACGACAAGGATGGCAGACTGCTGCAAGAGTTCAACAAGATCGAACCCCCTC
TGATCTTCGAGTGTAACCAAGCTTGCAGCTGTTGGAGGAACTGCAAGAATAGGGTCGTGCAG
TCCGGAATCAAGGTGAGACTGCAGCTGTATAGAACAGCTAAGATGGGATGGGGAGTCAGAG
CTCTGCAGACCATCCCCCAAGGCACATTCATCTGTGAGTACGTCGGCGAACTCATCAGCGAC
GCTGAGGCCGATGTGAGGGAGGACGACAGCTATCTCTTCGACCTCGACAACAAGGACGGCG
AGGTGTACTGCATCGACGCTAGATATTACGGCAACATCAGCAGATTCATCAACCACCTCTGC
GACCCCAATATCATCCCCGTGAGAGTGTTCATGCTCCATCAAGATCTGAGATTCCCTAGGAT
CGCCTTCTTCAGCTCTAGAGACATTAGAACCGGCGAGGAGCTGGGATTCGACTACGGCGACA
GGTTCTGGGACATCAAGAGCAAGTACTTCACATGCCAATGCGGCAGCGAGAAATGCAAGCA
TAGCGCCGAGGCCATTGCTCTGGAGCAGTCTAGACTGGCTAGGCTGGACCCTCACCCCGAGC
TGCTGCCCGAACTGGGATCTCTGCCTCCCGTGAATACCGGAGGTGGCGGATCGGGAGACAA
GAAGTACAGCATCGGCCTGGCCATCGGCACCAACAGCGTGGGCTGGGCCGTGATCACCGAC
GAGTACAAGGTGCCCAGCAAGAAGTTCAAGGTGCTGGGCAACACCGACCGGCACAGCATCA
AGAAGAACCTGATCGGCGCCCTGCTGTTCGACAGCGGCGAGACCGCCGAGGCCACCCGGCT
GAAGCGGACCGCCCGGCGGCGGTACACCCGGCGGAAGAACCGGATCTGCTACCTGCAGGAG
ATCTTCAGCAACGAGATGGCCAAGGTGGACGACAGCTTCTTCCACCGGCTGGAGGAGAGCT
TCCTGGTGGAGGAGGACAAGAAGCACGAGCGGCACCCCATCTTCGGCAACATCGTGGACGA
GGTGGCCTACCACGAGAAGTACCCCACCATCTACCACCTGCGGAAGAAGCTGGTGGACAGC
ACCGACAAGGCCGACCTGCGGCTGATCTACCTGGCCCTGGCCCACATGATCAAGTTCCGGGG
CCACTTCCTGATCGAGGGCGACCTGAACCCCGACAACAGCGACGTGGACAAGCTGTTCATCC
AGCTGGTGCAGACCTACAACCAGCTGTTCGAGGAGAACCCCATCAACGCCAGCGGCGTGGA
CGCCAAGGCCATCCTGAGCGCCCGGCTGAGCAAGAGCCGGCGGCTGGAGAACCTGATCGCC
CAGCTGCCCGGCGAGAAGAAGAACGGCCTGTTCGGCAACCTGATCGCCCTGAGCCTGGGCC
TGACCCCCAACTTCAAGAGCAACTTCGACCTGGCCGAGGACGCCAAGCTGCAGCTGAGCAA
GGACACCTACGACGACGACCTGGACAACCTGCTGGCCCAGATCGGCGACCAGTACGCCGAC
CTGTTCCTGGCCGCCAAGAACCTGAGCGACGCCATCCTGCTGAGCGACATCCTGCGGGTGAA
CACCGAGATCACCAAGGCCCCCCTGAGCGCCAGCATGATCAAGCGGTACGACGAGCACCAC
CAGGACCTGACCCTGCTGAAGGCCCTGGTGCGGCAGCAGCTGCCCGAGAAGTACAAGGAGA
TCTTCTTCGACCAGAGCAAGAACGGCTACGCCGGCTACATCGACGGCGGCGCCAGCCAGGA
GGAGTTCTACAAGTTCATCAAGCCCATCCTGGAGAAGATGGACGGCACCGAGGAGCTGCTG
GTGAAGCTGAACCGGGAGGACCTGCTGCGGAAGCAGCGGACCTTCGACAACGGCAGCATCC
CCCACCAGATCCACCTGGGCGAGCTGCACGCCATCCTGCGGCGGCAGGAGGACTTCTACCCC
TTCCTGAAGGACAACCGGGAGAAGATCGAGAAGATCCTGACCTTCCGGATCCCCTACTACGT
GGGCCCCCTGGCCCGGGGCAACAGCCGGTTCGCCTGGATGACCCGGAAATCCGAGGAGACC
ATCACCCCCTGGAACTTCGAGGAGGTGGTGGACAAGGGCGCCAGCGCCCAGAGCTTCATCG
AGCGGATGACCAACTTCGACAAGAACCTGCCCAACGAGAAGGTGCTGCCCAAGCACAGCCT
GCTGTACGAGTACTTCACCGTGTACAACGAGCTGACCAAGGTGAAGTACGTGACCGAGGGC
ATGCGGAAGCCCGCCTTCCTGAGCGGCGAGCAGAAGAAGGCCATCGTGGACCTGCTGTTCA
AGACCAACCGGAAGGTGACCGTGAAGCAGCTGAAGGAGGACTACTTCAAGAAGATCGAGTG
CTTCGACAGCGTGGAGATCAGCGGCGTGGAGGACCGGTTCAACGCCAGCCTGGGCACCTAC
CACGACCTGCTGAAGATCATCAAGGACAAGGACTTCCTGGACAACGAGGAGAACGAGGACA
TCCTGGAGGACATCGTGCTGACCCTGACCCTGTTCGAGGACCGGGAGATGATCGAGGAGCG
GCTGAAAACCTACGCCCACCTGTTCGACGACAAGGTGATGAAGCAGCTGAAGCGGCGGCGG
TACACCGGCTGGGGCCGGCTGAGCCGGAAGCTGATCAACGGCATCCGGGACAAGCAGAGCG
GCAAGACCATCCTGGACTTCCTGAAATCCGACGGCTTCGCCAACCGGAACTTCATGCAGCTG
ATCCACGACGACAGCCTGACCTTCAAGGAGGACATCCAGAAGGCCCAGGTGAGCGGCCAGG
GCGACAGCCTGCACGAGCACATCGCCAACCTGGCCGGCAGCCCCGCCATCAAGAAGGGCAT
CCTGCAGACCGTGAAGGTGGTGGACGAGCTGGTGAAGGTGATGGGCCGGCACAAGCCCGAG
AACATCGTGATCGAGATGGCCCGGGAGAACCAGACCACCCAGAAGGGCCAGAAGAACAGC
CGGGAGCGGATGAAGCGGATCGAGGAGGGCATCAAGGAGCTGGGCAGCCAGATCCTGAAG
GAGCACCCCGTGGAGAACACCCAGCTGCAGAACGAGAAGCTGTACCTGTACTACCTGCAGA
ACGGCCGGGACATGTACGTGGACCAGGAGCTGGACATCAACCGGCTGAGCGACTACGACGT
GGCCGCCATCGTGCCCCAGAGCTTCCTGAAGGACGACAGCATCGACAACAAGGTGCTGACC
CGGAGCGACAAGGCCCGGGGCAAGAGCGACAACGTGCCCAGCGAGGAGGTGGTGAAGAAG
ATGAAGAACTACTGGCGGCAGCTGCTGAACGCCAAGCTGATCACCCAGCGGAAGTTCGACA
ACCTGACCAAGGCCGAGCGGGGCGGCCTGAGCGAGCTGGACAAGGCCGGCTTCATCAAGCG
GCAGCTGGTGGAGACCCGGCAGATCACCAAGCACGTGGCCCAGATCCTGGACAGCCGGATG
AACACCAAGTACGACGAGAACGACAAGCTGATCCGGGAGGTGAAGGTGATCACCCTGAAAT
CCAAGCTGGTGAGCGACTTCCGGAAGGACTTCCAGTTCTACAAGGTGCGGGAGATCAACAA
CTACCACCACGCCCACGACGCCTACCTGAACGCCGTGGTGGGCACCGCCCTGATCAAGAAGT
ACCCCAAGCTGGAGAGCGAGTTCGTGTACGGCGACTACAAGGTGTACGACGTGCGGAAGAT
GATCGCCAAGAGCGAGCAGGAGATCGGCAAGGCCACCGCCAAGTACTTCTTCTACAGCAAC
ATCATGAACTTCTTCAAGACCGAGATCACCCTGGCCAACGGCGAGATCCGGAAGCGGCCCCT
GATCGAGACCAACGGCGAGACCGGCGAGATCGTGTGGGACAAGGGCCGGGACTTCGCCACC
GTGCGGAAGGTGCTGAGCATGCCCCAGGTGAACATCGTGAAGAAAACCGAGGTGCAGACCG
GCGGCTTCAGCAAGGAGAGCATCCTGCCCAAGCGGAACAGCGACAAGCTGATCGCCCGGAA
GAAGGACTGGGACCCCAAGAAGTACGGCGGCTTCGACAGCCCCACCGTGGCCTACAGCGTG
CTGGTGGTGGCCAAGGTGGAGAAGGGCAAGAGCAAGAAGCTGAAATCCGTGAAGGAGCTG
CTGGGCATCACCATCATGGAGCGGAGCAGCTTCGAGAAGAACCCCATCGACTTCCTGGAGG
CCAAGGGCTACAAGGAGGTGAAGAAGGACCTGATCATCAAGCTGCCCAAGTACAGCCTGTT
CGAGCTGGAGAACGGCCGGAAGCGGATGCTGGCCAGCGCCGGCGAGCTGCAGAAGGGCAA
CGAGCTGGCCCTGCCCAGCAAGTACGTGAACTTCCTGTACCTGGCCAGCCACTACGAGAAGC
TGAAGGGCAGCCCCGAGGACAACGAGCAGAAGCAGCTGTTCGTGGAGCAGCACAAGCACTA
CCTGGACGAGATCATCGAGCAGATCAGCGAGTTCAGCAAGCGGGTGATCCTGGCCGACGCC
AACCTGGACAAGGTGCTGAGCGCCTACAACAAGCACCGGGACAAGCCCATCCGGGAGCAGG
CCGAGAACATCATCCACCTGTTCACCCTGACCAACCTGGGCGCCCCCGCCGCCTTCAAGTAC
TTCGACACCACCATCGACCGGAAGCGGTACACCAGCACCAAGGAGGTGCTGGACGCCACCC
TGATCCACCAGAGCATCACCGGCCTGTACGAGACCCGGATCGACCTGAGCCAGCTGGGCGG
CGACAGCGGCGGCAAGCGGCCCGCCGCCACCAAGAAGGCCGGCCAGGCCAAGAAGAAGAA
GTCGGGCGGGGGTGGCTCAGGACAGACCGGCAAAAAGTCCGAAAAGGGCCCCGTGTGCTGG
AGGAAGAGGGTCAAGAGCGAGTACATGAGGCTGAGACAGCTCAAGAGATTTAGGAGAGCC
GATGAGGTGAAGTCCATGTTCTCCAGCAACAGACAAAAGATTCTGGAGAGGACCGAGATCC
TCAACCAAGAGTGGAAGCAGAGAAGAATCCAGCCCGTGCACATTCTGACCTCCGTGAGCTC
TCTGAGGGGCACAAGAGAATGCTCCGTCACCAGCGATCTGGACTTCCCCACACAAGTGATCC
CCCTCAAGACACTGAACGCTGTGGCCAGCGTGCCCATCATGTATAGCTGGTCCCCTCTGCAA
CAGAACTTCATGGTGGAGGACGAGACAGTGCTGCACAATATCCCCTACATGGGAGATGAGG
TGCTGGACCAAGACGGCACCTTTATTGAGGAGCTGATTAAAAACTACGATGGCAAGGTGCA
CGGCGATAGGGAGTGTGGCTTCATCAACGACGAGATCTTCGTCGAGCTGGTGAATGCTCTGG
GCCAGTATAATGACGATGATGACGACGATGACGGCGACGACCCCGAAGAGAGAGAGGAGA
AGCAAAAGGATCTGGAGGACCATAGGGACGACAAAGAGTCTAGACCTCCTAGAAAGTTCCC
CTCCGACAAGATCTTCGAAGCCATCTCCTCCATGTTCCCCGACAAGGGCACAGCCGAGGAAC
TGAAGGAGAAGTATAAGGAACTCACAGAGCAACAGCTGCCCGGAGCTCTGCCTCCCGAGTG
CACCCCTAACATCGACGGCCCCAACGCCAAGAGCGTGCAGAGGGAGCAATCCCTCCACAGC
TTCCATACCCTCTTCTGCAGAAGATGCTTTAAATACGATTGCTTTCTCCATCCTTTCCACGCC
ACACCCAACACCTACAAGAGGAAGAACACCGAAACCGCTCTGGACAATAAACCTTGCGGAC
CCCAGTGCTACCAGCATCTGGAAGGAGCCAAGGAATTTGCCGCTGCTCTGACAGCCGAGAG
AATTAAAACCCCTCCCAAAAGACCCGGCGGCAGAAGGAGGGGCAGACTGCCTAATAACAGC
AGCAGACCCAGCACCCCTACCATTAACGTGCTGGAATCCAAGGACACCGACAGCGATAGAG
AGGCCGGCACAGAAACCGGCGGAGAGAACAACGACAAGGAGGAGGAGGAGAAGAAAGAC
GAGACATCCTCCAGCAGCGAGGCTAATAGCAGATGCCAGACCCCTATCAAGATGAAACCTA
ATATCGAGCCCCCCGAGAATGTGGAGTGGAGCGGCGCTGAGGCCTCCATGTTTAGAGTGCTG
ATCGGAACCTACTACGACAACTTCTGCGCTATCGCTAGACTGATTGGCACCAAGACATGCAG
ACAAGTGTACGAGTTCAGAGTCAAGGAGAGCTCCATTATCGCCCCCGCCCCCGCCGAAGAT
GTGGACACCCCCCCCAGAAAGAAGAAAAGGAAGCATAGACTGTGGGCCGCCCACTGTAGAA
AGATCCAGCTCAAAAAGGACGGCAGCAGCAACCACGTGTACAACTATCAGCCTTGTGACCA
CCCCAGACAACCTTGTGATTCCAGCTGCCCTTGCGTGATCGCCCAGAACTTCTGCGAGAAGT
TCTGTCAGTGCAGCAGCGAGTGCCAAAATAGATTTCCCGGATGTAGGTGCAAAGCCCAGTGC
AATACCAAGCAGTGCCCTTGCTATCTGGCCGTGAGAGAGTGCGATCCCGATCTGTGTCTGAC
ATGTGGAGCTGCCGACCATTGGGACAGCAAGAATGTGAGCTGCAAGAACTGCAGCATCCAA
AGGGGAAGCAAAAAACATCTGCTGCTCGCCCCTTCCGATGTGGCCGGATGGGGAATCTTTAT
CAAGGACCCCGTCCAGAAAAACGAGTTCATTTCCGAGTATTGCGGCGAGATCATCAGCCAA
GACGAAGCTGATAGAAGAGGCAAAGTGTATGACAAATACATGTGCTCCTTCCTCTTCAACCT
CAATAATGATTTCGTGGTGGACGCCACAAGGAAGGGCAACAAGATTAGATTCGCCAACCAC
AGCGTCAATCCTAACTGCTATGCCAAGGTCATGATGGTCAACGGCGACCACAGAATTGGCAT
CTTCGCTAAGAGGGCCATCCAGACCGGCGAGGAACTGTTCTTCGACTATAGATACTCCCAAG
CCGACGCTCTGAAGTACGTGGGCATCGAGAGAGAGATGGAAATCCCCGGAGGTGGCGGATC
GGGAAAGCGGCCCGCCGCCACCAAGAAGGCCGGTCAGGCCAAGAAGAAGAAGGGCAGCTA
CCCCTACGACGTGCCCGACTACGCCTGAGCGGCCGCTTAATTAAGCTGCCTTCTGCGGGGCT
TGCCTTCTGGCCATGCCCTTCTTCTCTCCCTTGCACCTGTACCTCTTGGTCTTTGAATAAAGCC
TGAGTAGGAAGTCTAGAAAAAAAAAAAAAAAAAAAAAAAAAAAAAAAAAAAAAAAAAAA
AAAAAAAAAAAAAAAAAAAAAAAAAAAAAAAAAAAAA

In some embodiments, a site-specific disrupting agent comprises the amino acid sequence of SEQ ID NOs: 70 or 80. In some embodiments, a site-specific disrupting agent described herein comprises an amino acid sequence of SEQ ID NO: 70 or 80 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### G9A-dCas9-EZH2 Protein

(SEQ ID NO. 70)
MAPKKKRKVGIHGVPAASGGGGSGSAAIAEVLLNARCDLHAVNYHGDTPLHIAARESY
HDCVLLFLSRGANPELRNKEGDTAWDLTPERSDVWFALQLNRKLRLGVGNRAIRTEKIICRDVA
RGYENVPIPCVNGVDGEPCPEDYKYISENCETSTMNIDRNITHLQHCTCVDDCSSSNCLCGQLSIR
CWYDKDGRLLQEFNKIEPPLIFECNQACSCWRNCKNRVVQSGIKVRLQLYRTAKMGWGVRALQT
IPQGTFICEYVGELISDAEADVREDDSYLFDLDNKDGEVYCIDARYYGNISRFINHLCDPNIIPVR
VFMLHQDLRFPRIAFFSSRDIRTGEELGFDYGDRFWDIKSKYFTCQCGSEKCKHSAEAIALEQSRL
ARLDPHPELLPELGSLPPVNTGGGGSGDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAGYIDGGA
SQEEFYKFIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLTLFE
DREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRD
MYVDQELDINRLSDYDVAAIVPQSFLKDDSIDNKVLTRSDKARGKSDNVPSEEVVKKMKNYWR
QLLNAKLITQRKFDNLTKAERGGLSELDKAGFIKRQLVETRQITKHVAQILDSRMNTKYDENDK
LIREVKVITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLFELE
NGRKRMLASAGELQKGNELALPSKYVNFLYLASHYEKLKGSPEDNEQKQLFVEQHKHYLDEIIE
QISEFSKRVILADANLDKVLSAYNKHRDKPIREQAENIIHLFTLTNLGAPAAFKYFDTTIDRKRYT
STKEVLDATLIHQSITGLYETRIDLSQLGGDSGGKRPAATKKAGQAKKKKSGGGGSGQTGKKSE
KGPVCWRKRVKSEYMRLRQLKRFRRADEVKSMFSSNRQKILERTEILNQEWKQRRIQPVHILTS
VSSLRGTRECSVTSDLDFPTQVIPLKTLNAVASVPIMYSWSPLQQNFMVEDETVLHNIPYMGDEV
LDQDGTFIEELIKNYDGKVHGDRECGFINDEIFVELVNALGQYNDDDDDDDGDDPEEREEKQKD
LEDHRDDKESRPPRKFPSDKIFEAISSMFPDKGTAEELKEKYKELTEQQLPGALPPECTPNIDGPN
AKSVQREQSLHSFHTLFCRRCFKYDCFLHPFHATPNTYKRKNTETALDNKPCGPQCYQHLEGAK
EFAAALTAERIKTPPKRPGGRRRGRLPNNSSRPSTPTINVLESKDTDSDREAGTETGGENNDKEEE
EKKDETSSSSEANSRCQTPIKMKPNIEPPENVEWSGAEASMFRVLIGTYYDNFCAIARLIGTKTCR
QVYEFRVKESSIIAPAPAEDVDTPPRKKKRKHRLWAAHCRKIQLKKDGSSNHVYNYQPCDHPRQ
PCDSSCPCVIAQNFCEKFCQCSSECQNRFPGCRCKAQCNTKQCPCYLAVRECDPDLCLTCGAAD
HWDSKNVSCKNCSIQRGSKKHLLLAPSDVAGWGIFIKDPVQKNEFISEYCGEIISQDEADRRGKV
YDKYMCSFLFNLNNDFVVDATRKGNKIRFANHSVNPNCYAKVMMVNGDHRIGIFAKRAIQTGE
ELFFDYRYSQADALKYVGIEREMEIPGGGGSGKRPAATKKAGQAKKKKGSYPYDVPDYA

G9A-dCas9-EZH2 Protein without HA Tag

(SEQ ID NO. 80)
MAPKKKRKVGIHGVPAASGGGGSGSAAIAEVLLNARCDLHAVNYHGDTPLHIAARESY
HDCVLLFLSRGANPELRNKEGDTAWDLTPERSDVWFALQLNRKLRLGVGNRAIRTEKIICRDVA
RGYENVPIPCVNGVDGEPCPEDYKYISENCETSTMNIDRNITHLQHCTCVDDCSSSNCLCGQLSIR
CWYDKDGRLLQEFNKIEPPLIFECNQACSCWRNCKNRVVQSGIKVRLQLYRTAKMGWGVRALQT
IPQGTFICEYVGELISDAEADVREDDSYLFDLDNKDGEVYCIDARYYGNISRFINHLCDPNIIPVR
VFMLHQDLRFPRIAFFSSRDIRTGEELGFDYGDRFWDIKSKYFTCQCGSEKCKHSAEAIALEQSRL
ARLDPHPELLPELGSLPPVNTGGGGSGDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLTLFE
DREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRD
MYVDQELDINRLSDYDVAAIVPQSFLKDDSIDNKVLTRSDKARGKSDNVPSEEVVKKMKNYWR
QLLNAKLITQRKFDNLTKAERGGLSELDKAGFIKRQLVETRQITKHVAQILDSRMNTKYDENDK
LIREVKVITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLFELE
NGRKRMLASAGELQKGNELALPSKYVNFLYLASHYEKLKGSPEDNEQKQLFVEQHKHYLDEIIE
QISEFSKRVILADANLDKVLSAYNKHRDKPIREQAENIIHLFTLTNLGAPAAFKYFDTTIDRKRYT
STKEVLDATLIHQSITGLYETRIDLSQLGGDSGGKRPAATKKAGQAKKKKSGGGGSGQTGKKSE
KGPVCWRKRVKSEYMRLRQLKRFRRADEVKSMFSSNRQKILERTEILNQEWKQRRIQPVHILTS
VSSLRGTRECSVTSDLDFPTQVIPLKTLNAVASVPIMYSWSPLQQNFMVEDETVLHNIPYMGDEV
LDQDGTFIEELIKNYDGKVHGDRECGFINDEIFVELVNALGQYNDDDDDDDGDDPEEREEKQKD
LEDHRDDKESRPPRKFPSDKIFEAISSMFPDKGTAEELKEKYKELTEQQLPGALPPECTPNIDGPN
AKSVQREQSLHSFHTLFCRRCFKYDCFLHPFHATPNTYKRKNTETALDNKPCGPQCYQHLEGAK
EFAAALTAERIKTPPKRPGGRRRGRLPNNSSRPSTPTINVLESKDTDSDREAGTETGGENNDKEEE
EKKDETSSSSEANSRCQTPIKMKPNIEPPENVEWSGAEASMFRVLIGTYYDNFCAIARLIGTKTCR
QVYEFRVKESSIIAPAPAEDVDTPPRKKKRKHRLWAAHCRKIQLKKDGSSNHVYNYQPCDHPRQ
PCDSSCPCVIAQNFCEKFCQCSSECQNRFPGCRCKAQCNTKQCPCYLAVRECDPDLCLTCGAAD
HWDSKNVSCKNCSIQRGSKKHLLLAPSDVAGWGIFIKDPVQKNEFISEYCGEIISQDEADRRGKV
YDKYMCSFLFNLNNDFVVDATRKGNKIRFANHSVNPNCYAKVMMVNGDHRIGIFAKRAIQTGE
ELFFDYRYSQADALKYVGIEREMEIPGGGGSGKRPAATKKAGQAKKKKGS

In some embodiments, a site-specific disrupting agent comprises a targeting moiety (e.g., comprising dCas9, e.g., an S. aureus dCas9), a first effector moiety comprising G9A; e.g., a G9A domain, and a second effector moiety comprising KRAB, e.g., a KRAB domain. In some embodiments, a site-specific disrupting agent comprises a targeting moiety comprising dCas9, e.g., an S. pyogenes dCas9, e.g., a mutant S. pyogenes Cas9, e.g., Cas9m4; a first effector moiety comprising G9A, e.g., a G9A domain; and a second effector moiety comprising KRAB, e.g., a KRAB domain. In some embodiments, the site-specific disrupting agent is encoded by the nucleic acid sequence of SEQ ID NO: 71 (e.g., mRNA encoding the site-specific disrupting agent). In some embodiments, a nucleic acid described herein comprises a nucleic acid sequence of SEQ ID NO: 71 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9A-dCas9-KRAB (MR-29942) mRNA Sequence

(SEQ ID NO. 71)
AGGAAATAAGAGAGAAAAGAAGAGTAAGAAGAAATATAAGAGCCACCATGGCCCC
CAAGAAGAAGCGGAAGGTGGGCATCCACGGCGTGCCCGCCGCCTCGGGCGGGGGTGGCTCA
GGAAGCGCCGCTATCGCCGAAGTGCTGCTGAATGCCAGATGCGATCTGCATGCCGTGAACTA
CCACGGCGACACCCCTCTGCATATCGCCGCTAGAGAGAGCTACCATGACTGTGTGCTGCTGT
TTCTGAGCAGAGGCGCCAATCCCGAGCTCAGAAACAAAGAGGGCGACACCGCTTGGGATCT
GACACCCGAGAGATCCGACGTGTGGTTCGCTCTGCAACTGAATAGAAAACTGAGACTGGGC
GTCGGCAATAGGGCCATTAGAACCGAGAAGATCATCTGTAGGGACGTGGCTAGGGGCTACG
AGAACGTGCCCATCCCTTGTGTGAACGGAGTGGATGGAGAGCCTTGCCCCGAGGATTACAA
ATACATCAGCGAGAACTGCGAAACCTCCACCATGAATATCGATAGAAACATTACACACCTCC
AGCACTGTACATGCGTGGACGATTGCAGCAGCAGCAACTGTCTGTGCGGCCAACTGAGCATC
AGATGCTGGTACGACAAGGATGGCAGACTGCTGCAAGAGTTCAACAAGATCGAACCCCCTC
TGATCTTCGAGTGTAACCAAGCTTGCAGCTGTTGGAGGAACTGCAAGAATAGGGTCGTGCAG
TCCGGAATCAAGGTGAGACTGCAGCTGTATAGAACAGCTAAGATGGGATGGGGAGTCAGAG
CTCTGCAGACCATCCCCCAAGGCACATTCATCTGTGAGTACGTCGGCGAACTCATCAGCGAC
GCTGAGGCCGATGTGAGGGAGGACGACAGCTATCTCTTCGACCTCGACAACAAGGACGGCG
AGGTGTACTGCATCGACGCTAGATATTACGGCAACATCAGCAGATTCATCAACCACCTCTGC
GACCCCAATATCATCCCCGTGAGAGTGTTCATGCTCCATCAAGATCTGAGATTCCCTAGGAT
CGCCTTCTTCAGCTCTAGAGACATTAGAACCGGCGAGGAGCTGGGATTCGACTACGGCGACA
GGTTCTGGGACATCAAGAGCAAGTACTTCACATGCCAATGCGGCAGCGAGAAATGCAAGCA
TAGCGCCGAGGCCATTGCTCTGGAGCAGTCTAGACTGGCTAGGCTGGACCCTCACCCCGAGC
TGCTGCCCGAACTGGGATCTCTGCCTCCCGTGAATACCGGAGGTGGCGGATCGGGAGACAA
GAAGTACAGCATCGGCCTGGCCATCGGCACCAACAGCGTGGGCTGGGCCGTGATCACCGAC
GAGTACAAGGTGCCCAGCAAGAAGTTCAAGGTGCTGGGCAACACCGACCGGCACAGCATCA
AGAAGAACCTGATCGGCGCCCTGCTGTTCGACAGCGGCGAGACCGCCGAGGCCACCCGGCT
GAAGCGGACCGCCCGGCGGCGGTACACCCGGCGGAAGAACCGGATCTGCTACCTGCAGGAG
ATCTTCAGCAACGAGATGGCCAAGGTGGACGACAGCTTCTTCCACCGGCTGGAGGAGAGCT
TCCTGGTGGAGGAGGACAAGAAGCACGAGCGGCACCCCATCTTCGGCAACATCGTGGACGA
GGTGGCCTACCACGAGAAGTACCCCACCATCTACCACCTGCGGAAGAAGCTGGTGGACAGC
ACCGACAAGGCCGACCTGCGGCTGATCTACCTGGCCCTGGCCCACATGATCAAGTTCCGGGG
CCACTTCCTGATCGAGGGCGACCTGAACCCCGACAACAGCGACGTGGACAAGCTGTTCATCC
AGCTGGTGCAGACCTACAACCAGCTGTTCGAGGAGAACCCCATCAACGCCAGCGGCGTGGA
CGCCAAGGCCATCCTGAGCGCCCGGCTGAGCAAGAGCCGGCGGCTGGAGAACCTGATCGCC
CAGCTGCCCGGCGAGAAGAAGAACGGCCTGTTCGGCAACCTGATCGCCCTGAGCCTGGGCC
TGACCCCCAACTTCAAGAGCAACTTCGACCTGGCCGAGGACGCCAAGCTGCAGCTGAGCAA
GGACACCTACGACGACGACCTGGACAACCTGCTGGCCCAGATCGGCGACCAGTACGCCGAC
CTGTTCCTGGCCGCCAAGAACCTGAGCGACGCCATCCTGCTGAGCGACATCCTGCGGGTGAA
CACCGAGATCACCAAGGCCCCCCTGAGCGCCAGCATGATCAAGCGGTACGACGAGCACCAC
CAGGACCTGACCCTGCTGAAGGCCCTGGTGCGGCAGCAGCTGCCCGAGAAGTACAAGGAGA
TCTTCTTCGACCAGAGCAAGAACGGCTACGCCGGCTACATCGACGGCGGCGCCAGCCAGGA
GGAGTTCTACAAGTTCATCAAGCCCATCCTGGAGAAGATGGACGGCACCGAGGAGCTGCTG
GTGAAGCTGAACCGGGAGGACCTGCTGCGGAAGCAGCGGACCTTCGACAACGGCAGCATCC
CCCACCAGATCCACCTGGGCGAGCTGCACGCCATCCTGCGGCGGCAGGAGGACTTCTACCCC
TTCCTGAAGGACAACCGGGAGAAGATCGAGAAGATCCTGACCTTCCGGATCCCCTACTACGT
GGGCCCCCTGGCCCGGGGCAACAGCCGGTTCGCCTGGATGACCCGGAAATCCGAGGAGACC
ATCACCCCCTGGAACTTCGAGGAGGTGGTGGACAAGGGCGCCAGCGCCCAGAGCTTCATCG
AGCGGATGACCAACTTCGACAAGAACCTGCCCAACGAGAAGGTGCTGCCCAAGCACAGCCT
GCTGTACGAGTACTTCACCGTGTACAACGAGCTGACCAAGGTGAAGTACGTGACCGAGGGC
ATGCGGAAGCCCGCCTTCCTGAGCGGCGAGCAGAAGAAGGCCATCGTGGACCTGCTGTTCA
AGACCAACCGGAAGGTGACCGTGAAGCAGCTGAAGGAGGACTACTTCAAGAAGATCGAGTG
CTTCGACAGCGTGGAGATCAGCGGCGTGGAGGACCGGTTCAACGCCAGCCTGGGCACCTAC
CACGACCTGCTGAAGATCATCAAGGACAAGGACTTCCTGGACAACGAGGAGAACGAGGACA
TCCTGGAGGACATCGTGCTGACCCTGACCCTGTTCGAGGACCGGGAGATGATCGAGGAGCG
GCTGAAAACCTACGCCCACCTGTTCGACGACAAGGTGATGAAGCAGCTGAAGCGGCGGCGG
TACACCGGCTGGGGCCGGCTGAGCCGGAAGCTGATCAACGGCATCCGGGACAAGCAGAGCG
GCAAGACCATCCTGGACTTCCTGAAATCCGACGGCTTCGCCAACCGGAACTTCATGCAGCTG
ATCCACGACGACAGCCTGACCTTCAAGGAGGACATCCAGAAGGCCCAGGTGAGCGGCCAGG
GCGACAGCCTGCACGAGCACATCGCCAACCTGGCCGGCAGCCCCGCCATCAAGAAGGGCAT
CCTGCAGACCGTGAAGGTGGTGGACGAGCTGGTGAAGGTGATGGGCCGGCACAAGCCCGAG
AACATCGTGATCGAGATGGCCCGGGAGAACCAGACCACCCAGAAGGGCCAGAAGAACAGC
CGGGAGCGGATGAAGCGGATCGAGGAGGGCATCAAGGAGCTGGGCAGCCAGATCCTGAAG
GAGCACCCCGTGGAGAACACCCAGCTGCAGAACGAGAAGCTGTACCTGTACTACCTGCAGA
ACGGCCGGGACATGTACGTGGACCAGGAGCTGGACATCAACCGGCTGAGCGACTACGACGT
GGCCGCCATCGTGCCCCAGAGCTTCCTGAAGGACGACAGCATCGACAACAAGGTGCTGACC
CGGAGCGACAAGGCCCGGGGCAAGAGCGACAACGTGCCCAGCGAGGAGGTGGTGAAGAAG
ATGAAGAACTACTGGCGGCAGCTGCTGAACGCCAAGCTGATCACCCAGCGGAAGTTCGACA
ACCTGACCAAGGCCGAGCGGGGCGGCCTGAGCGAGCTGGACAAGGCCGGCTTCATCAAGCG
GCAGCTGGTGGAGACCCGGCAGATCACCAAGCACGTGGCCCAGATCCTGGACAGCCGGATG
AACACCAAGTACGACGAGAACGACAAGCTGATCCGGGAGGTGAAGGTGATCACCCTGAAAT
CCAAGCTGGTGAGCGACTTCCGGAAGGACTTCCAGTTCTACAAGGTGCGGGAGATCAACAA
CTACCACCACGCCCACGACGCCTACCTGAACGCCGTGGTGGGCACCGCCCTGATCAAGAAGT
ACCCCAAGCTGGAGAGCGAGTTCGTGTACGGCGACTACAAGGTGTACGACGTGCGGAAGAT
GATCGCCAAGAGCGAGCAGGAGATCGGCAAGGCCACCGCCAAGTACTTCTTCTACAGCAAC
ATCATGAACTTCTTCAAGACCGAGATCACCCTGGCCAACGGCGAGATCCGGAAGCGGCCCCT
GATCGAGACCAACGGCGAGACCGGCGAGATCGTGTGGGACAAGGGCCGGGACTTCGCCACC
GTGCGGAAGGTGCTGAGCATGCCCCAGGTGAACATCGTGAAGAAAACCGAGGTGCAGACCG
GCGGCTTCAGCAAGGAGAGCATCCTGCCCAAGCGGAACAGCGACAAGCTGATCGCCCGGAA
GAAGGACTGGGACCCCAAGAAGTACGGCGGCTTCGACAGCCCCACCGTGGCCTACAGCGTG
CTGGTGGTGGCCAAGGTGGAGAAGGGCAAGAGCAAGAAGCTGAAATCCGTGAAGGAGCTG
CTGGGCATCACCATCATGGAGCGGAGCAGCTTCGAGAAGAACCCCATCGACTTCCTGGAGG
CCAAGGGCTACAAGGAGGTGAAGAAGGACCTGATCATCAAGCTGCCCAAGTACAGCCTGTT
CGAGCTGGAGAACGGCCGGAAGCGGATGCTGGCCAGCGCCGGCGAGCTGCAGAAGGGCAA
CGAGCTGGCCCTGCCCAGCAAGTACGTGAACTTCCTGTACCTGGCCAGCCACTACGAGAAGC
TGAAGGGCAGCCCCGAGGACAACGAGCAGAAGCAGCTGTTCGTGGAGCAGCACAAGCACTA
CCTGGACGAGATCATCGAGCAGATCAGCGAGTTCAGCAAGCGGGTGATCCTGGCCGACGCC
AACCTGGACAAGGTGCTGAGCGCCTACAACAAGCACCGGGACAAGCCCATCCGGGAGCAGG
CCGAGAACATCATCCACCTGTTCACCCTGACCAACCTGGGCGCCCCCGCCGCCTTCAAGTAC
TTCGACACCACCATCGACCGGAAGCGGTACACCAGCACCAAGGAGGTGCTGGACGCCACCC
TGATCCACCAGAGCATCACCGGCCTGTACGAGACCCGGATCGACCTGAGCCAGCTGGGCGG
CGACAGCGGCGGCAAGCGGCCCGCCGCCACCAAGAAGGCCGGCCAGGCCAAGAAGAAGAA
GTCGGGCGGGGGTGGCTCAGACGCTAAGTCTCTGACCGCTTGGAGCAGAACACTGGTCACCT
TCAAGGACGTGTTCGTCGACTTCACAAGAGAGGAGTGGAAACTGCTGGACACCGCCCAGCA
GATCCTCTATAGAAACGTCATGCTGGAGAACTACAAGAATCTGGTGTCTCTGGGCTACCAGC
TGACCAAGCCCGACGTGATTCTGAGGCTGGAGAAGGGCGAGGAGCCTTGGCTGGTGGAGAG
AGAGATCCACCAAGAAACCCACCCCGACAGCGAAACCGCCTTCGAGATCAAGAGCAGCGTG
GGAGGTGGCGGATCGGGAAAGCGGCCCGCCGCCACCAAGAAGGCCGGTCAGGCCAAGAAG
AAGAAGGGCAGCTACCCCTACGACGTGCCCGACTACGCCTGAGCGGCCGCTTAATTAAGCT
GCCTTCTGCGGGGCTTGCCTTCTGGCCATGCCCTTCTTCTCTCCCTTGCACCTGTACCTCTTGG
TCTTTGAATAAAGCCTGAGTAGGAAGTCTAGAAAAAAAAAAAAAAAAAAAAAAAAAAAAA
AAAAAAAAAAAAAAAAAAAAAAAAAAAAAAAAAAAAAAAAAAAAAAAAAAA

In some embodiments, a site-specific disrupting agent comprises the amino acid sequence of SEQ ID NOs: 72 or 81. In some embodiments, a site-specific disrupting agent described herein comprises an amino acid sequence of SEQ ID NO: 72 or 81 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### G9A-dCas9-KRAB Protein

(SEQ ID NO. 72)
MAPKKKRKVGIHGVPAASGGGGSGSAAIAEVLLNARCDLHAVNYHGDTPLHIAARESY
HDCVLLFLSRGANPELRNKEGDTAWDLTPERSDVWFALQLNRKLRLGVGNRAIRTEKIICRDVA
RGYENVPIPCVNGVDGEPCPEDYKYISENCETSTMNIDRNITHLQHCTCVDDCSSSNCLCGQLSIR
CWYDKDGRLLQEFNKIEPPLIFECNQACSCWRNCKNRVVQSGIKVRLQLYRTAKMGWGVRALQT
IPQGTFICEYVGELISDAEADVREDDSYLFDLDNKDGEVYCIDARYYGNISRFINHLCDPNIIPVR
VFMLHQDLRFPRIAFFSSRDIRTGEELGFDYGDRFWDIKSKYFTCQCGSEKCKHSAEAIALEQSRL
ARLDPHPELLPELGSLPPVNTGGGGSGDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLTLFE
DREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRD
MYVDQELDINRLSDYDVAAIVPQSFLKDDSIDNKVLTRSDKARGKSDNVPSEEVVKKMKNYWR
QLLNAKLITQRKFDNLTKAERGGLSELDKAGFIKRQLVETRQITKHVAQILDSRMNTKYDENDK
LIREVKVITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLFELE
NGRKRMLASAGELQKGNELALPSKYVNFLYLASHYEKLKGSPEDNEQKQLFVEQHKHYLDEIIE
QISEFSKRVILADANLDKVLSAYNKHRDKPIREQAENIIHLFTLTNLGAPAAFKYFDTTIDRKRYT
STKEVLDATLIHQSITGLYETRIDLSQLGGDSGGKRPAATKKAGQAKKKKSGGGGSDAKSLTAW
SRTLVTFKDVFVDFTREEWKLLDTAQQILYRNVMLENYKNLVSLGYQLTKPDVILRLEKGEEPW
LVEREIHQETHPDSETAFEIKSSVGGGGSGKRPAATKKAGQAKKKKGSYPYDVPDYA

G9A-dCas9-KRAB Protein without HA Tag

(SEQ ID NO. 81)
MAPKKKRKVGIHGVPAASGGGGSGSAAIAEVLLNARCDLHAVNYHGDTPLHIAARESY
HDCVLLFLSRGANPELRNKEGDTAWDLTPERSDVWFALQLNRKLRLGVGNRAIRTEKIICRDVA
RGYENVPIPCVNGVDGEPCPEDYKYISENCETSTMNIDRNITHLQHCTCVDDCSSSNCLCGQLSIR
CWYDKDGRLLQEFNKIEPPLIFECNQACSCWRNCKNRVVQSGIKVRLQLYRTAKMGWGVRALQT
IPQGTFICEYVGELISDAEADVREDDSYLFDLDNKDGEVYCIDARYYGNISRFINHLCDPNIIPVR
VFMLHQDLRFPRIAFFSSRDIRTGEELGFDYGDRFWDIKSKYFTCQCGSEKCKHSAEAIALEQSRL
ARLDPHPELLPELGSLPPVNTGGGGSGDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLTLFE
DREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRD
MYVDQELDINRLSDYDVAAIVPQSFLKDDSIDNKVLTRSDKARGKSDNVPSEEVVKKMKNYWR
QLLNAKLITQRKFDNLTKAERGGLSELDKAGFIKRQLVETRQITKHVAQILDSRMNTKYDENDK
LIREVKVITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLFELE
NGRKRMLASAGELQKGNELALPSKYVNFLYLASHYEKLKGSPEDNEQKQLFVEQHKHYLDEIIE
QISEFSKRVILADANLDKVLSAYNKHRDKPIREQAENIIHLFTLTNLGAPAAFKYFDTTIDRKRYT
STKEVLDATLIHQSITGLYETRIDLSQLGGDSGGKRPAATKKAGQAKKKKSGGGGSDAKSLTAW
SRTLVTFKDVFVDFTREEWKLLDTAQQILYRNVMLENYKNLVSLGYQLTKPDVILRLEKGEEPW
LVEREIHQETHPDSETAFEIKSSVGGGGSGKRPAATKKAGQAKKKKGS

In some embodiments, a site-specific disrupting agent comprises a targeting moiety (e.g., comprising dCas9, e.g., an S. aureus dCas9), a first effector moiety comprising EZH2; e.g., an EZH2 domain, and a second effector moiety comprising KRAB, e.g., a KRAB domain. In some embodiments, a site-specific disrupting agent comprises a targeting moiety comprising dCas9, e.g., an S. pyogenes dCas9, e.g., a mutant S. pyogenes Cas9, e.g., Cas9m4; a first effector moiety comprising EZH2, e.g., an EZH2 domain; and a second effector moiety comprising KRAB, e.g., a KRAB domain. In some embodiments, the site-specific disrupting agent is encoded by the nucleic acid sequence of SEQ ID NO: 85 (e.g., mRNA encoding the site-specific disrupting agent). In some embodiments, a nucleic acid described herein comprises a nucleic acid sequence of SEQ ID NO: 85 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2-dCas9-KRAB (MR-29948) mRNA sequence

(SEQ ID NO. 85)
AGGAAATAAGAGAGAAAAGAAGAGTAAGAAGAAATATAAGAGCCACCATGGCCCC
CAAGAAGAAGCGGAAGGTGGGCGGCAGCGGCGGCAGCGGCCAGACCGGCAAGAAGAGCGA
GAAGGGCCCCGTGTGCTGGCGGAAGCGGGTGAAGAGCGAGTACATGCGGCTGCGGCAGCTG
AAGCGGTTCCGGCGGGCCGACGAGGTGAAGAGCATGTTCAGCAGCAACCGGCAGAAGATCC
TGGAGCGGACCGAGATCCTGAACCAGGAGTGGAAGCAGCGGCGAATCCAGCCCGTGCACAT
CCTGACCAGCGTGAGCAGCCTGCGGGGCACCCGGGAGTGCAGCGTGACCAGCGACCTGGAC
TTCCCCACCCAGGTGATCCCCCTAAAGACCCTGAACGCCGTGGCCAGCGTGCCCATCATGTA
CAGCTGGAGCCCCCTGCAGCAGAACTTCATGGTGGAGGACGAGACCGTGCTGCACAACATC
CCCTACATGGGCGACGAGGTGCTGGACCAGGACGGCACCTTCATCGAGGAGCTGATCAAGA
ACTACGACGGCAAGGTGCACGGCGACCGGGAGTGCGGCTTCATCAACGACGAGATCTTCGT
GGAGCTGGTGAACGCCCTGGGCCAGTACAACGACGACGACGACGACGACGACGGCGACGA
CCCCGAGGAGCGGGAGGAGAAGCAGAAGGACCTGGAGGACCACCGGGACGACAAGGAGAG
CCGGCCCCCCCGGAAGTTCCCCAGCGACAAGATCTTCGAGGCCATCAGCAGCATGTTCCCCG
ACAAGGGCACCGCCGAGGAGCTGAAGGAGAAGTACAAGGAGCTGACCGAGCAGCAGCTGC
CCGGCGCCCTGCCCCCCGAGTGCACCCCCAACATCGACGGCCCCAACGCCAAGAGCGTGCA
GCGGGAGCAGAGCCTGCACAGCTTCCACACCCTGTTCTGCCGGCGGTGCTTCAAGTACGACT
GCTTCCTGCACCCCTTCCACGCCACCCCCAACACCTACAAGCGGAAGAACACCGAGACCGCC
CTGGACAACAAGCCCTGCGGCCCCCAGTGCTACCAGCACCTGGAGGGCGCCAAGGAGTTCG
CCGCCGCCCTGACCGCCGAGCGGATCAAGACCCCCCCCAAGCGGCCCGGCGGCCGGCGGCG
GGGCCGGCTGCCCAACAACAGCAGCCGGCCCAGCACCCCCACCATCAACGTGCTGGAGAGC
AAGGACACCGACAGCGACCGGGAGGCCGGCACCGAGACCGGCGGCGAGAACAACGACAAG
GAGGAGGAGGAGAAGAAGGACGAGACCAGCAGCAGCAGCGAGGCCAACAGCCGGTGCCAG
ACCCCCATCAAGATGAAGCCCAACATCGAGCCCCCCGAGAACGTGGAGTGGAGCGGCGCCG
AGGCCAGCATGTTCCGGGTGCTGATCGGCACCTACTACGACAACTTCTGCGCCATCGCCCGG
CTGATCGGCACCAAGACCTGCCGGCAGGTGTACGAGTTCCGGGTGAAGGAGAGCAGCATCA
TCGCCCCCGCCCCCGCCGAGGACGTGGACACCCCCCCCCGGAAGAAGAAGCGGAAGCACCG
GCTGTGGGCCGCCCACTGCCGGAAGATCCAGCTGAAGAAGGACGGCAGCAGCAACCACGTG
TACAACTACCAGCCCTGCGACCACCCCCGGCAGCCCTGCGACAGCAGCTGCCCCTGCGTGAT
CGCCCAGAACTTCTGCGAGAAGTTCTGCCAGTGCAGCAGCGAGTGCCAGAACCGGTTCCCCG
GCTGCCGGTGCAAGGCCCAGTGCAACACCAAGCAGTGCCCCTGCTACCTGGCCGTGCGGGA
GTGCGACCCCGACCTGTGCCTGACCTGCGGCGCCGCCGACCACTGGGACAGCAAGAACGTG
AGCTGCAAGAACTGCAGCATCCAGCGGGGCAGCAAGAAGCACCTGCTGCTGGCCCCCAGCG
ACGTGGCCGGCTGGGGCATCTTCATCAAGGACCCCGTGCAGAAGAACGAGTTCATCAGCGA
GTACTGCGGCGAGATCATCAGCCAGGACGAGGCCGACCGGCGGGGCAAGGTGTACGACAAG
TACATGTGCAGCTTCCTGTTCAACCTGAACAACGACTTCGTGGTGGACGCCACCCGGAAGGG
CAACAAGATCCGGTTCGCCAACCACAGCGTGAACCCCAACTGCTACGCCAAGGTGATGATG
GTGAACGGCGACCACCGGATCGGCATCTTCGCCAAGCGGGCCATCCAGACCGGCGAGGAGC
TGTTCTTCGACTACCGGTACAGCCAGGCCGACGCCCTGAAGTACGTGGGCATCGAGCGGGA
GATGGAGATCCCCAGCACCGGCGGCAGCGGCGGCAGCGGCGGCAGCGGCGGCAGCGGCGG
CAGCGGCCGACCCGACAAGAAGTACAGCATCGGCCTGGCCATCGGCACCAACAGCGTGGGC
TGGGCCGTGATCACCGACGAGTACAAGGTGCCCAGCAAGAAGTTCAAGGTGCTGGGCAACA
CCGACCGGCACAGCATCAAGAAGAACCTGATCGGCGCCCTGCTGTTCGACAGCGGCGAGAC
CGCCGAGGCCACCCGGCTGAAGCGGACCGCCCGGCGGCGGTACACCCGGCGGAAGAACCGG
ATCTGCTACCTGCAGGAGATCTTCAGCAACGAGATGGCCAAGGTGGACGACAGCTTCTTCCA
CCGGCTGGAGGAGAGCTTCCTGGTGGAGGAGGACAAGAAGCACGAGCGGCACCCCATCTTC
GGCAACATCGTGGACGAGGTGGCCTACCACGAGAAGTACCCCACCATCTACCACCTGCGGA
AGAAGCTGGTGGACAGCACCGACAAGGCCGACCTGCGGCTGATCTACCTGGCCCTGGCCCA
CATGATCAAGTTCCGGGGCCACTTCCTGATCGAGGGCGACCTGAACCCCGACAACAGCGAC
GTGGACAAGCTGTTCATCCAGCTGGTGCAGACCTACAACCAGCTGTTCGAGGAGAACCCCAT
CAACGCCAGCGGCGTGGACGCCAAGGCCATCCTGAGCGCCCGGCTGAGCAAGAGCCGGCGG
CTGGAGAACCTGATCGCCCAGCTGCCCGGCGAGAAGAAGAACGGCCTGTTCGGCAACCTGA
TCGCCCTGAGCCTGGGCCTGACCCCCAACTTCAAGAGCAACTTCGACCTGGCCGAGGACGCC
AAGCTGCAGCTGAGCAAGGACACCTACGACGACGACCTGGACAACCTGCTGGCCCAGATCG
GCGACCAGTACGCCGACCTGTTCCTGGCCGCCAAGAACCTGAGCGACGCCATCCTGCTGAGC
GACATCCTGCGGGTGAACACCGAGATCACCAAGGCCCCCCTGAGCGCCAGCATGATCAAGC
GGTACGACGAGCACCACCAGGACCTGACCCTGCTGAAGGCCCTGGTGCGGCAGCAGCTGCC
CGAGAAGTACAAGGAGATCTTCTTCGACCAGAGCAAGAACGGCTACGCCGGCTACATCGAC
GGCGGCGCCAGCCAGGAGGAGTTCTACAAGTTCATCAAGCCCATCCTGGAGAAGATGGACG
GCACCGAGGAGCTGCTGGTGAAGCTGAACCGGGAGGACCTGCTGCGGAAGCAGCGGACCTT
CGACAACGGCAGCATCCCCCACCAGATCCACCTGGGCGAGCTGCACGCCATCCTGCGGCGG
CAGGAGGACTTCTACCCCTTCCTGAAGGACAACCGGGAGAAGATCGAGAAGATCCTGACCT
TCCGGATCCCCTACTACGTGGGCCCCCTGGCCCGGGGCAACAGCCGGTTCGCCTGGATGACC
CGGAAATCCGAGGAGACCATCACCCCCTGGAACTTCGAGGAGGTGGTGGACAAGGGCGCCA
GCGCCCAGAGCTTCATCGAGCGGATGACCAACTTCGACAAGAACCTGCCCAACGAGAAGGT
GCTGCCCAAGCACAGCCTGCTGTACGAGTACTTCACCGTGTACAACGAGCTGACCAAGGTGA
AGTACGTGACCGAGGGCATGCGGAAGCCCGCCTTCCTGAGCGGCGAGCAGAAGAAGGCCAT
CGTGGACCTGCTGTTCAAGACCAACCGGAAGGTGACCGTGAAGCAGCTGAAGGAGGACTAC
TTCAAGAAGATCGAGTGCTTCGACAGCGTGGAGATCAGCGGCGTGGAGGACCGGTTCAACG
CCAGCCTGGGCACCTACCACGACCTGCTGAAGATCATCAAGGACAAGGACTTCCTGGACAA
CGAGGAGAACGAGGACATCCTGGAGGACATCGTGCTGACCCTGACCCTGTTCGAGGACCGG
GAGATGATCGAGGAGCGGCTGAAAACCTACGCCCACCTGTTCGACGACAAGGTGATGAAGC
AGCTGAAGCGGCGGCGGTACACCGGCTGGGGCCGGCTGAGCCGGAAGCTGATCAACGGCAT
CCGGGACAAGCAGAGCGGCAAGACCATCCTGGACTTCCTGAAATCCGACGGCTTCGCCAAC
CGGAACTTCATGCAGCTGATCCACGACGACAGCCTGACCTTCAAGGAGGACATCCAGAAGG
CCCAGGTGAGCGGCCAGGGCGACAGCCTGCACGAGCACATCGCCAACCTGGCCGGCAGCCC
CGCCATCAAGAAGGGCATCCTGCAGACCGTGAAGGTGGTGGACGAGCTGGTGAAGGTGATG
GGCCGGCACAAGCCCGAGAACATCGTGATCGAGATGGCCCGGGAGAACCAGACCACCCAGA
AGGGCCAGAAGAACAGCCGGGAGCGGATGAAGCGGATCGAGGAGGGCATCAAGGAGCTGG
GCAGCCAGATCCTGAAGGAGCACCCCGTGGAGAACACCCAGCTGCAGAACGAGAAGCTGTA
CCTGTACTACCTGCAGAACGGCCGGGACATGTACGTGGACCAGGAGCTGGACATCAACCGG
CTGAGCGACTACGACGTGGCCGCCATCGTGCCCCAGAGCTTCCTGAAGGACGACAGCATCG
ACAACAAGGTGCTGACCCGGAGCGACAAGGCCCGGGGCAAGAGCGACAACGTGCCCAGCG
AGGAGGTGGTGAAGAAGATGAAGAACTACTGGCGGCAGCTGCTGAACGCCAAGCTGATCAC
CCAGCGGAAGTTCGACAACCTGACCAAGGCCGAGCGGGGCGGCCTGAGCGAGCTGGACAAG
GCCGGCTTCATCAAGCGGCAGCTGGTGGAGACCCGGCAGATCACCAAGCACGTGGCCCAGA
TCCTGGACAGCCGGATGAACACCAAGTACGACGAGAACGACAAGCTGATCCGGGAGGTGAA
GGTGATCACCCTGAAATCCAAGCTGGTGAGCGACTTCCGGAAGGACTTCCAGTTCTACAAGG
TGCGGGAGATCAACAACTACCACCACGCCCACGACGCCTACCTGAACGCCGTGGTGGGCAC
CGCCCTGATCAAGAAGTACCCCAAGCTGGAGAGCGAGTTCGTGTACGGCGACTACAAGGTG
TACGACGTGCGGAAGATGATCGCCAAGAGCGAGCAGGAGATCGGCAAGGCCACCGCCAAGT
ACTTCTTCTACAGCAACATCATGAACTTCTTCAAGACCGAGATCACCCTGGCCAACGGCGAG
ATCCGGAAGCGGCCCCTGATCGAGACCAACGGCGAGACCGGCGAGATCGTGTGGGACAAGG
GCCGGGACTTCGCCACCGTGCGGAAGGTGCTGAGCATGCCCCAGGTGAACATCGTGAAGAA
AACCGAGGTGCAGACCGGCGGCTTCAGCAAGGAGAGCATCCTGCCCAAGCGGAACAGCGAC
AAGCTGATCGCCCGGAAGAAGGACTGGGACCCCAAGAAGTACGGCGGCTTCGACAGCCCCA
CCGTGGCCTACAGCGTGCTGGTGGTGGCCAAGGTGGAGAAGGGCAAGAGCAAGAAGCTGAA
ATCCGTGAAGGAGCTGCTGGGCATCACCATCATGGAGCGGAGCAGCTTCGAGAAGAACCCC
ATCGACTTCCTGGAGGCCAAGGGCTACAAGGAGGTGAAGAAGGACCTGATCATCAAGCTGC
CCAAGTACAGCCTGTTCGAGCTGGAGAACGGCCGGAAGCGGATGCTGGCCAGCGCCGGCGA
GCTGCAGAAGGGCAACGAGCTGGCCCTGCCCAGCAAGTACGTGAACTTCCTGTACCTGGCC
AGCCACTACGAGAAGCTGAAGGGCAGCCCCGAGGACAACGAGCAGAAGCAGCTGTTCGTGG
AGCAGCACAAGCACTACCTGGACGAGATCATCGAGCAGATCAGCGAGTTCAGCAAGCGGGT
GATCCTGGCCGACGCCAACCTGGACAAGGTGCTGAGCGCCTACAACAAGCACCGGGACAAG
CCCATCCGGGAGCAGGCCGAGAACATCATCCACCTGTTCACCCTGACCAACCTGGGCGCCCC
CGCCGCCTTCAAGTACTTCGACACCACCATCGACCGGAAGCGGTACACCAGCACCAAGGAG
GTGCTGGACGCCACCCTGATCCACCAGAGCATCACCGGCCTGTACGAGACCCGGATCGACCT
GAGCCAGCTGGGCGGCGACAGCGGCGGCAAGCGGCCCGCCGCCACCAAGAAGGCCGGCCA
GGCCAAGAAGAAGAAGTCGGGCGGGGGTGGCTCAGACGCTAAGTCTCTGACCGCTTGGAGC
AGAACACTGGTCACCTTCAAGGACGTGTTCGTCGACTTCACAAGAGAGGAGTGGAAACTGC
TGGACACCGCCCAGCAGATCCTCTATAGAAACGTCATGCTGGAGAACTACAAGAATCTGGT
GTCTCTGGGCTACCAGCTGACCAAGCCCGACGTGATTCTGAGGCTGGAGAAGGGCGAGGAG
CCTTGGCTGGTGGAGAGAGAGATCCACCAAGAAACCCACCCCGACAGCGAAACCGCCTTCG
AGATCAAGAGCAGCGTGGGAGGTGGCGGATCGGGAAAGCGGCCCGCCGCCACCAAGAAGG
CCGGTCAGGCCAAGAAGAAGAAGGGCAGCTACCCCTACGACGTGCCCGACTACGCCTGAGC
GGCCGCTTAATTAAGCTGCCTTCTGCGGGGCTTGCCTTCTGGCCATGCCCTTCTTCTCTCCCTT
GCACCTGTACCTCTTGGTCTTTGAATAAAGCCTGAGTAGGAAGTCTAGAAAAAAAAAAAAA
AAAAAAAAAAAAAAAAAAAAAAAAAAAAAAAAAAAAAAAAAAAAAAAAAAAAAAAAAA
AAAAAAAAA

In some embodiments, a site-specific disrupting agent comprises the amino acid sequence of SEQ ID NOs: 86 or 82. In some embodiments, a site-specific disrupting agent described herein comprises an amino acid sequence of SEQ ID NO: 86 or 82 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### EZH2-dCas9-KRAB Protein

(SEQ ID NO. 86)
MAPKKKRKVGGSGGSGQTGKKSEKGPVCWRKRVKSEYMRLRQLKRFRRADEVKSMFSSN
RQKILERTEILNQEWKQRRIQPVHILTSVSSLRGTRECSVTSDLDFPTQVIPLKTLNAVASVPIM
YSWSPLQQNFMVEDETVLHNIPYMGDEVLDQDGTFIEELIKNYDGKVHGDRECGFINDEIFVELV
NALGQYNDDDDDDDGDDPEEREEKQKDLEDHRDDKESRPPRKFPSDKIFEAISSMFPDKGTAEE
LKEKYKELTEQQLPGALPPECTPNIDGPNAKSVQREQSLHSFHTLFCRRCFKYDCFLHPFHATPNT
YKRKNTETALDNKPCGPQCYQHLEGAKEFAAALTAERIKTPPKRPGGRRRGRLPNNSSRPSTPTI
NVLESKDTDSDREAGTETGGENNDKEEEEKKDETSSSSEANSRCQTPIKMKPNIEPPENVEWSGA
EASMFRVLIGTYYDNFCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCSSECQNRFPGCRCKA
QCNTKQCPCYLAVRECDPDLCLTCGAADHWDSKNVSCKNCSIQRGSKKHLLLAPSDVAGWGIFI
KDPVQKNEFISEYCGEIISQDEADRRGKVYDKYMCSFLFNLNNDFVVDATRKGNKIRFANHSVN
PNCYAKVMMVNGDHRIGIFAKRAIQTGEELFFDYRYSQADALKYVGIEREMEIPSTGGSGGSGG
SGGSGGSGRPDKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFIQ
LVQTYNQLFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEITKAPLS
ASMIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDKNLPNEKVLPKHSLL
YEYFTVYNELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARENQTTQKG
QKNSRERMKRIEEGIKELGSQILKEHPVENTQLQNEKLYLYYLQNGRDMYVDQELDINRLSDYD
VAAIVPQSFLKDDSIDNKVLTRSDKARGKSDNVPSEEVVKKMKNYWRQLLNAKLITQRKFDNLT
KAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVLSMPQVNI
VKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKG
NELALPSKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQSITGL
YETRIDLSQLGGDSGGKRPAATKKAGQAKKKKSGGGGSDAKSLTAWSRTLVTFKDVFVDFTREE
WKLLDTAQQILYRNVMLENYKNLVSLGYQLTKPDVILRLEKGEEPWLVEREIHQETHPDSETAF
EIKSSVGGGGSGKRPAATKKAGQAKKKKGSYPYDVPDYA

EZH2-dCas9-KRAB Protein without HA Tag

(SEQ ID NO. 82)
MAPKKKRKVGGSGGSGQTGKKSEKGPVCWRKRVKSEYMRLRQLKRFRRADEVKSMFSSN
RQKILERTEILNQEWKQRRIQPVHILTSVSSLRGTRECSVTSDLDFPTQVIPLKTLNAVASVPIM
YSWSPLQQNFMVEDETVLHNIPYMGDEVLDQDGTFIEELIKNYDGKVHGDRECGFINDEIFVELV
NALGQYNDDDDDDDGDDPEEREEKQKDLEDHRDDKESRPPRKFPSDKIFEAISSMFPDKGTAEE
LKEKYKELTEQQLPGALPPECTPNIDGPNAKSVQREQSLHSFHTLFCRRCFKYDCFLHPFHATPNT
YKRKNTETALDNKPCGPQCYQHLEGAKEFAAALTAERIKTPPKRPGGRRRGRLPNNSSRPSTPTI
NVLESKDTDSDREAGTETGGENNDKEEEEKKDETSSSSEANSRCQTPIKMKPNIEPPENVEWSGA
EASMFRVLIGTYYDNFCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCSSECQNRFPGCRCKA
QCNTKQCPCYLAVRECDPDLCLTCGAADHWDSKNVSCKNCSIQRGSKKHLLLAPSDVAGWGIFI
KDPVQKNEFISEYCGEIISQDEADRRGKVYDKYMCSFLFNLNNDFVVDATRKGNKIRFANHSVN
PNCYAKVMMVNGDHRIGIFAKRAIQTGEELFFDYRYSQADALKYVGIEREMEIPSTGGSGGSGG
SGGSGGSGRPDKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFIQ
LVQTYNQLFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEITKAPLS
ASMIKRYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSLL
YEYFTVYNELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARENQTTQKG
QKNSRERMKRIEEGIKELGSQILKEHPVENTQLQNEKLYLYYLQNGRDMYVDQELDINRLSDYD
VAAIVPQSFLKDDSIDNKVLTRSDKARGKSDNVPSEEVVKKMKNYWRQLLNAKLITQRKFDNLT
KAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVLSMPQVNI
VKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKG
NELALPSKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQSITGL
YETRIDLSQLGGDSGGKRPAATKKAGQAKKKKSGGGGSDAKSLTAWSRTLVTFKDVFVDFTREE
WKLLDTAQQILYRNVMLENYKNLVSLGYQLTKPDVILRLEKGEEPWLVEREIHQETHPDSETAF
EIKSSVGGGGSGKRPAATKKAGQAKKKKGS

In some embodiments, a site-specific disrupting agent comprises a CRISPR/Cas molecule comprising Cas9. In some embodiments, the site-specific disrupting agent is encoded by the nucleic acid sequence of SEQ ID NO: 217 (e.g., mRNA encoding the site-specific disrupting agent). In some embodiments, a nucleic acid described herein comprises a nucleic acid sequence of SEQ ID NO: 217 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

Cas9 mRNA (MR-28127)

(SEQ ID NO: 217)
AGGAAAUAAGAGAGAAAAGAAGAGUAAGAAGAAAUAUAAGAGCCACCAUGGCCC
CCAAGAAGAAGCGGAAGGUGGGCAUCCACGGCGUGCCCGCCGCCGACAAGAAGUACAGCA
UCGGCCUGGACAUCGGCACCAACAGCGUGGGCUGGGCCGUGAUCACCGACGAGUACAAGG
UGCCCAGCAAGAAGUUCAAGGUGCUGGGCAACACCGACCGGCACAGCAUCAAGAAGAACC
UGAUCGGCGCCCUGCUGUUCGACAGCGGCGAGACCGCCGAGGCCACCCGGCUGAAGCGGA
CCGCCCGGCGGCGGUACACCCGGCGGAAGAACCGGAUCUGCUACCUGCAGGAGAUCUUCA
GCAACGAGAUGGCCAAGGUGGACGACAGCUUCUUCCACCGGCUGGAGGAGAGCUUCCUGG
UGGAGGAGGACAAGAAGCACGAGCGGCACCCCAUCUUCGGCAACAUCGUGGACGAGGUGG
CCUACCACGAGAAGUACCCCACCAUCUACCACCUGCGGAAGAAGCUGGUGGACAGCACCG
ACAAGGCCGACCUGCGGCUGAUCUACCUGGCCCUGGCCCACAUGAUCAAGUUCCGGGGCC
ACUUCCUGAUCGAGGGCGACCUGAACCCCGACAACAGCGACGUGGACAAGCUGUUCAUCC
AGCUGGUGCAGACCUACAACCAGCUGUUCGAGGAGAACCCCAUCAACGCCAGCGGCGUGG
ACGCCAAGGCCAUCCUGAGCGCCCGGCUGAGCAAGAGCCGGCGGCUGGAGAACCUGAUCG
CCCAGCUGCCCGGCGAGAAGAAGAACGGCCUGUUCGGCAACCUGAUCGCCCUGAGCCUGG
GCCUGACCCCCAACUUCAAGAGCAACUUCGACCUGGCCGAGGACGCCAAGCUGCAGCUGA
GCAAGGACACCUACGACGACGACCUGGACAACCUGCUGGCCCAGAUCGGCGACCAGUACG
CCGACCUGUUCCUGGCCGCCAAGAACCUGAGCGACGCCAUCCUGCUGAGCGACAUCCUGC
GGGUGAACACCGAGAUCACCAAGGCCCCCCUGAGCGCCAGCAUGAUCAAGCGGUACGACG
AGCACCACCAGGACCUGACCCUGCUGAAGGCCCUGGUGCGGCAGCAGCUGCCCGAGAAGU
ACAAGGAGAUCUUCUUCGACCAGAGCAAGAACGGCUACGCCGGCUACAUCGACGGCGGCG
CCAGCCAGGAGGAGUUCUACAAGUUCAUCAAGCCCAUCCUGGAGAAGAUGGACGGCACCG
AGGAGCUGCUGGUGAAGCUGAACCGGGAGGACCUGCUGCGGAAGCAGCGGACCUUCGACA
ACGGCAGCAUCCCCCACCAGAUCCACCUGGGCGAGCUGCACGCCAUCCUGCGGCGGCAGG
AGGACUUCUACCCCUUCCUGAAGGACAACCGGGAGAAGAUCGAGAAGAUCCUGACCUUCC
GGAUCCCCUACUACGUGGGCCCCCUGGCCCGGGGCAACAGCCGGUUCGCCUGGAUGACCC
GGAAAUCCGAGGAGACCAUCACCCCCUGGAACUUCGAGGAGGUGGUGGACAAGGGCGCCA
GCGCCCAGAGCUUCAUCGAGCGGAUGACCAACUUCGACAAGAACCUGCCCAACGAGAAGG
UGCUGCCCAAGCACAGCCUGCUGUACGAGUACUUCACCGUGUACAACGAGCUGACCAAGG
UGAAGUACGUGACCGAGGGCAUGCGGAAGCCCGCCUUCCUGAGCGGCGAGCAGAAGAAGG
CCAUCGUGGACCUGCUGUUCAAGACCAACCGGAAGGUGACCGUGAAGCAGCUGAAGGAGG
ACUACUUCAAGAAGAUCGAGUGCUUCGACAGCGUGGAGAUCAGCGGCGUGGAGGACCGG
UUCAACGCCAGCCUGGGCACCUACCACGACCUGCUGAAGAUCAUCAAGGACAAGGACUUC
CUGGACAACGAGGAGAACGAGGACAUCCUGGAGGACAUCGUGCUGACCCUGACCCUGUUC
GAGGACCGGGAGAUGAUCGAGGAGCGGCUGAAAACCUACGCCCACCUGUUCGACGACAAG
GUGAUGAAGCAGCUGAAGCGGCGGCGGUACACCGGCUGGGGCCGGCUGAGCCGGAAGCUG
AUCAACGGCAUCCGGGACAAGCAGAGCGGCAAGACCAUCCUGGACUUCCUGAAAUCCGAC
GGCUUCGCCAACCGGAACUUCAUGCAGCUGAUCCACGACGACAGCCUGACCUUCAAGGAG
GACAUCCAGAAGGCCCAGGUGAGCGGCCAGGGCGACAGCCUGCACGAGCACAUCGCCAAC
CUGGCCGGCAGCCCCGCCAUCAAGAAGGGCAUCCUGCAGACCGUGAAGGUGGUGGACGAG
CUGGUGAAGGUGAUGGGCCGGCACAAGCCCGAGAACAUCGUGAUCGAGAUGGCCCGGGA
GAACCAGACCACCCAGAAGGGCCAGAAGAACAGCCGGGAGCGGAUGAAGCGGAUCGAGGA
GGGCAUCAAGGAGCUGGGCAGCCAGAUCCUGAAGGAGCACCCCGUGGAGAACACCCAGCU
GCAGAACGAGAAGCUGUACCUGUACUACCUGCAGAACGGCCGGGACAUGUACGUGGACCA
GGAGCUGGACAUCAACCGGCUGAGCGACUACGACGUGGACCACAUCGUGCCCCAGAGCUU
CCUGAAGGACGACAGCAUCGACAACAAGGUGCUGACCCGGAGCGACAAGAACCGGGGCAA
GAGCGACAACGUGCCCAGCGAGGAGGUGGUGAAGAAGAUGAAGAACUACUGGCGGCAGC
UGCUGAACGCCAAGCUGAUCACCCAGCGGAAGUUCGACAACCUGACCAAGGCCGAGCGGG
GCGGCCUGAGCGAGCUGGACAAGGCCGGCUUCAUCAAGCGGCAGCUGGUGGAGACCCGGC
AGAUCACCAAGCACGUGGCCCAGAUCCUGGACAGCCGGAUGAACACCAAGUACGACGAGA
ACGACAAGCUGAUCCGGGAGGUGAAGGUGAUCACCCUGAAAUCCAAGCUGGUGAGCGAC
UUCCGGAAGGACUUCCAGUUCUACAAGGUGCGGGAGAUCAACAACUACCACCACGCCCAC
GACGCCUACCUGAACGCCGUGGUGGGCACCGCCCUGAUCAAGAAGUACCCCAAGCUGGAG
AGCGAGUUCGUGUACGGCGACUACAAGGUGUACGACGUGCGGAAGAUGAUCGCCAAGAG
CGAGCAGGAGAUCGGCAAGGCCACCGCCAAGUACUUCUUCUACAGCAACAUCAUGAACUU
CUUCAAGACCGAGAUCACCCUGGCCAACGGCGAGAUCCGGAAGCGGCCCCUGAUCGAGAC
CAACGGCGAGACCGGCGAGAUCGUGUGGGACAAGGGCCGGGACUUCGCCACCGUGCGGAA
GGUGCUGAGCAUGCCCCAGGUGAACAUCGUGAAGAAAACCGAGGUGCAGACCGGCGGCUU
CAGCAAGGAGAGCAUCCUGCCCAAGCGGAACAGCGACAAGCUGAUCGCCCGGAAGAAGGA
CUGGGACCCCAAGAAGUACGGCGGCUUCGACAGCCCCACCGUGGCCUACAGCGUGCUGGU
GGUGGCCAAGGUGGAGAAGGGCAAGAGCAAGAAGCUGAAAUCCGUGAAGGAGCUGCUGG
GCAUCACCAUCAUGGAGCGGAGCAGCUUCGAGAAGAACCCCAUCGACUUCCUGGAGGCCA
AGGGCUACAAGGAGGUGAAGAAGGACCUGAUCAUCAAGCUGCCCAAGUACAGCCUGUUC
GAGCUGGAGAACGGCCGGAAGCGGAUGCUGGCCAGCGCCGGCGAGCUGCAGAAGGGCAAC
GAGCUGGCCCUGCCCAGCAAGUACGUGAACUUCCUGUACCUGGCCAGCCACUACGAGAAG
CUGAAGGGCAGCCCCGAGGACAACGAGCAGAAGCAGCUGUUCGUGGAGCAGCACAAGCAC
UACCUGGACGAGAUCAUCGAGCAGAUCAGCGAGUUCAGCAAGCGGGUGAUCCUGGCCGAC
GCCAACCUGGACAAGGUGCUGAGCGCCUACAACAAGCACCGGGACAAGCCCAUCCGGGAG
CAGGCCGAGAACAUCAUCCACCUGUUCACCCUGACCAACCUGGGCGCCCCCGCCGCCUUCA
AGUACUUCGACACCACCAUCGACCGGAAGCGGUACACCAGCACCAAGGAGGUGCUGGACG
CCACCCUGAUCCACCAGAGCAUCACCGGCCUGUACGAGACCCGGAUCGACCUGAGCCAGC
UGGGCGGCGACAGCGGCGGCAAGCGGCCCGCCGCCACCAAGAAGGCCGGCCAGGCCAAGA
AGAAGAAGGGCAGCUACCCCUACGACGUGCCCGACUACGCCUGAGCGGCCGCUUAAUUAA
GCUGCCUUCUGCGGGGCUUGCCUUCUGGCCAUGCCCUUCUUCUCUCCCUUGCACCUGUAC
CUCUUGGUCUUUGAAUAAAGCCUGAGUAGGAAGUCUAGAAAAAAAAAAAAAAAAAAAAA
AAAAAAAAAAAAAAAAAAAAAAAAAAAAAAAAAAAAAAAAAAAAAAAAAAAAAAAAAA
A

In some embodiments, a site-specific disrupting agent comprises the amino acid sequence of SEQ ID NOs: 218 or 84. In some embodiments, a site-specific disrupting agent described herein comprises an amino acid sequence of SEQ ID NO: 218 or 84 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

### Cas9 Protein Sequence (Corresponding to MR-28127)

(SEQ ID NO: 218)
MAPKKKRKVGIHGVPAADKKYSIGLDIGTNSVGWAVITDEYKVPSKKFKVLGNTDRHSI
KKNLIGALLFDSGETAEATRLKRTARRRYTRRKNRICYLQEIFSNEMAKVDDSFFHRLEESFLVEE
DKKHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNRKVTVKQL
KEDYFKKIECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFANRNFMQ
LIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYV
DQELDINRLSDYDVDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYFDTTIDRKRYTSTKEVL
DATLIHQSITGLYETRIDLSQLGGDSGGKRPAATKKAGQAKKKKGSYPYDVPDYA

Cas9 Protein Sequence (Corresponding to MR-28127) without HA Tag

(SEQ ID NO: 84)
MAPKKKRKVGIHGVPAADKKYSIGLDIGTNSVGWAVITDEYKVPSKKFKVLGNTDRHSI
KKNLIGALLFDSGETAEATRLKRTARRRYTRRKNRICYLQEIFSNEMAKVDDSFFHRLEESFLVEE
DKKHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNRKVTVKQL
KEDYFKKIECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFANRNFMQ
LIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYV
DQELDINRLSDYDVDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYFDTTIDRKRYTSTKEVL
DATLIHQSITGLYETRIDLSQLGGDSGGKRPAATKKAGQAKKKKGS

In some embodiments, a site-specific disrupting agent comprises a nuclear localization sequence (NLS). In some embodiments, the site-specific disrupting agent comprises an NLS, e.g., an SV40 NLS at the N-terminus. In some embodiments, the site-specific disrupting agent comprises an NLS, e.g., an SV40 NLS at the C-terminus. In some embodiments, the site-specific disrupting agent comprises an NLS, e.g., a nucleoplasmin NLS at the C-terminus. In some embodiments, the site-specific disrupting agent comprises a first NLS at the N-terminus and a second NLS at the C-terminus. In some embodiments the first and the second NLS have the same sequence. In some embodiments, the first and the second NLS have different sequences. In some embodiments, the site-specific disrupting agent comprises a first NLS at the N-terminus, a second NLS, and a third NLS at the C-terminus. In some embodiments, at least two NLSs have the same sequence. In some embodiments, the first and the second NLS have the same sequence and the third NLS has a different sequence than the first and the second NLS. In some embodiments, the site-specific disrupting agent comprises an SV40 NLS, e.g., the site-specific disrupting agent comprises a sequence according to PKKKRK (SEQ ID NO: 63). In some embodiments, the site-specific disrupting agent comprises a nucleoplasmin NLS, e.g., the site-specific disrupting agent comprises a sequence of KRPAATKKAGQAKKK (SEQ ID NO: 64). In some embodiments, the site-specific disrupting agent comprises a C-terminal sequence comprising one or more of, e.g., any one or both of: a nucleoplasmin nuclear localization sequence and an HA-tag. In some embodiments, the site-specific disrupting agent comprises an epitope tag, e.g., an HA tag: YPYDVPDYA (SEQ ID NO: 65). In some embodiments, the site-specific disrupting agent may comprise two copies of the epitope tag.

While an epitope tag is useful in many research contexts, it is sometimes desirable to omit an epitope tag in a therapeutic context. Accordingly, in some embodiments, the site-specific disrupting agent lacks an epitope tag. In some embodiments, a site-specific disrupting agent described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking the HA tag of SEQ ID NO: 65. In some embodiments, a nucleic acid described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking a region encoding the HA tag of SEQ ID NO: 65. In some embodiments, the site-specific disrupting agent does not comprise an NLS. In some embodiments, the site-specific disrupting agent does not comprise an epitope tag. In some embodiments the site-specific disrupting agent does not comprise an HA tag. In some embodiments, the site-specific disrupting agent does not comprise an HA tag sequence according to SEQ ID NO: 65.

In some embodiments, a site-specific disrupting agent comprises a targeting moiety comprising a Zn finger molecule and an effector moiety comprising EZH2 or a functional fragment or variant thereof. In some embodiments, the site-specific disrupting agent is encoded by the nucleic acid sequence of SEQ ID NOs: 219, 220, 222, 223, 233, or 234, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, a site-specific disrupting agent comprises a targeting moiety comprising a Zn finger molecule and an effector moiety comprising DNMT3 (e.g., DNMT3a or DNMT3L) or a functional fragment or variant thereof. In some embodiments, the site-specific disrupting agent is encoded by the nucleic acid sequence of SEQ ID NOs: 221, 231, or 236-239, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, a site-specific disrupting agent comprises a targeting moiety comprising a Zn finger molecule and an effector moiety comprising G9A or a functional fragment or variant thereof. In some embodiments, the site-specific disrupting agent is encoded by the nucleic acid sequence of SEQ ID NOs: 224, 225, or 227-230, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, a site-specific disrupting agent comprises a targeting moiety comprising a Zn finger molecule and an effector moiety comprising HDAC8 or a functional fragment or variant thereof. In some embodiments, the site-specific disrupting agent is encoded by the nucleic acid sequence of SEQ ID NOs: 226, 232, 235, or 240-242, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

TABLE 3
Additional Exemplary Site-Specific Disrupting Agent Encoding Sequences

| Ref. | | |
|---|---|---|
| number | | |
| and (SEQ | Agent | |
| ID NO) | Description | Sequence |
| MR- | EZH2-CXCL1 | AGGAAATAAGAGAGAAAAGAAGAGTAAGAAGAAATATAAGAGCCACCATGGCCCCCAAGAAGAA |
| 29950 | ZF6T7 | TGCTGGCGGAAGCGGGTGAAGAGCGAGTACATGCGGCTGCGGCAGCTGAAGCGGTTCCGGCGGG |
| (SEQ ID | | CGCGGAAGGTGGGCGGCAGCGGCGGCAGCGGCCAGACCGGCAAGAAGAGCGAGAAGGGCCCCGT |
| NO: 219) | | GCGACGAGGTGAAGAGCATGTTCAGCAGCAACCGGCAGAAGATCCTGGAGCGGACCGAGATCCT |
| | | GAACCAGGAGTGGAAGCAGCGGCGAATCCAGCCCGTGCACATCCTGACCAGCGTGAGCAGCCTG |
| | | CGGGGCACCCGGGAGTGCAGCGTGACCAGCGACCTGGACTTCCCCACCCAGGTGATCCCCCTAA |
| | | AGACCCTGAACGCCGTGGCCAGCGTGCCCATCATGTACAGCTGGAGCCCCCTGCAGCAGAACTT |
| | | CATGGTGGAGGACGAGACCGTGCTGCACAACATCCCCTACATGGGCGACGAGGTGCTGGACCAG |
| | | GACGGCACCTTCATCGAGGAGCTGATCAAGAACTACGACGGCAAGGTGCACGGCGACCGGGAGT |
| | | GCGGCTTCATCAACGACGAGATCTTCGTGGAGCTGGTGAACGCCCTGGGCCAGTACAACGACGA |
| | | CGACGACGACGACGACGGCGACGACCCCGAGGAGCGGGAGGAGAAGCAGAAGGACCTGGAGGAC |
| | | CACCGGGACGACAAGGAGAGCCGGCCCCCCCGGAAGTTCCCCAGCGACAAGATCTTCGAGGCCA |
| | | TCAGCAGCATGTTCCCCGACAAGGGCACCGCCGAGGAGCTGAAGGAGAAGTACAAGGAGCTGAC |
| | | CGAGCAGCAGCTGCCCGGCGCCCTGCCCCCCGAGTGCACCCCCAACATCGACGGCCCCAACGCC |
| | | AAGAGCGTGCAGCGGGAGCAGAGCCTGCACAGCTTCCACACCCTGTTCTGCCGGCGGTGCTTCA |
| | | AGTACGACTGCTTCCTGCACCCCTTCCACGCCACCCCCAACACCTACAAGCGGAAGAACACCGA |
| | | GACCGCCCTGGACAACAAGCCCTGCGGCCCCCAGTGCTACCAGCACCTGGAGGGCGCCAAGGAG |
| | | TTCGCCGCCGCCCTGACCGCCGAGCGGATCAAGACCCCCCCCAAGCGGCCCGGCGGCCGGCGGC |
| | | GGGGCCGGCTGCCCAACAACAGCAGCCGGCCCAGCACCCCCACCATCAACGTGCTGGAGAGCAA |
| | | GGACACCGACAGCGACCGGGAGGCCGGCACCGAGACCGGCGGCGAGAACAACGACAAGGAGGAG |
| | | GAGGAGAAGAAGGACGAGACCAGCAGCAGCAGCGAGGCCAACAGCCGGTGCCAGACCCCCATCA |
| | | AGATGAAGCCCAACATCGAGCCCCCCGAGAACGTGGAGTGGAGCGGCGCCGAGGCCAGCATGTT |
| | | CCGGGTGCTGATCGGCACCTACTACGACAACTTCTGCGCCATCGCCCGGCTGATCGGCACCAAG |
| | | ACCTGCCGGCAGGTGTACGAGTTCCGGGTGAAGGAGAGCAGCATCATCGCCCCCGCCCCCGCCG |
| | | AGGACGTGGACACCCCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACCAGCCCTGCGACCAC |
| | | CCCCGGCAGCCCTGCGACAGCAGCTGCCCCTGCGTGATCGCCCAGAACTTCTGCGAGAAGTTCT |
| | | GCCAGTGCAGCAGCGAGTGCCAGAACCGGTTCCCCGGCTGCCGGTGCAAGGCCCAGTGCAACAC |
| | | CAAGCAGTGCCCCTGCTACCTGGCCGTGCGGGAGTGCGACCCCGACCTGTGCCTGACCTGCGGC |
| | | GCCGCCGACCACTGGGACAGCAAGAACGTGAGCTGCAAGAACTGCAGCATCCAGCGGGGCAGCA |
| | | AGAAGCACCTGCTGCTGGCCCCCAGCGACGTGGCCGGCTGGGGCATCTTCATCAAGGACCCCGT |
| | | GCAGAAGAACGAGTTCATCAGCGAGTACTGCGGCGAGATCATCAGCCAGGACGAGGCCGACCGG |
| | | CGGGGCAAGGTGTACGACAAGTACATGTGCAGCTTCCTGTTCAACCTGAACAACGACTTCGTGG |
| | | TGGACGCCACCCGGAAGGGCAACAAGATCCGGTTCGCCAACCACAGCGTGAACCCCAACTGCTA |
| | | CGCCAAGGTGATGATGGTGAACGGCGACCACCGGATCGGCATCTTCGCCAAGCGGGCCATCCAG |
| | | ACCGGCGAGGAGCTGTTCTTCGACTACCGGTACAGCCAGGCCGACGCCCTGAAGTACGTGGGCA |
| | | TCGAGCGGGAGATGGAGATCCCCGGCAGCAGCGGATCCCTGGAGCCCGGCGAAAAGCCTTACAA |
| | | GTGTCCCGAGTGCGGAAAGAGCTTCAGCAGAGCCGATAATCTGACCGAGCACCAAAGGACCCAC |
| | | ACCGGAGAGAAGCCTTATAAGTGTCCCGAATGCGGCAAAAGCTTTTCTAGAAGCGATCATCTGA |
| | | CCAACCACCAGAGGACACACACCGGAGAAAAACCTTACAAATGCCCCGAGTGCGGCAAAAGCTT |
| | | CTCCCAGAGCAGCAATCTGGTGAGACACCAAAGGACCCACACCGGCGAAAAACCCTATAAATGC |
| | | CCCGAATGTGGCAAGAGCTTTAGCACATCCGGCGAGCTGGTGAGGCATCAAAGAACACATACCG |
| | | GCGAGAAGCCCTACAAGTGCCCCGAGTGTGGAAAAAGCTTCAGCACCCACCTCGATCTGATCAG |
| | | ACACCAGAGGACCCATACCGGAGAGAAACCCTACAAATGTCCCGAGTGCGGAAAGTCCTTTAGC |
| | | CAGCTGGCCCATCTGAGAGCTCATCAAAGGACACACACCGGCGAGAAGCCTTACAAGTGTCCCG |
| | | AGTGCGGAAAATCCTTCTCCCAACTGGCCCATCTGAGGGCCCACCAGAGAACCCACACCGGCAA |
| | | AAAGACCTCCGCTAGCGGCAGCGGCGGCGGCAGCGGCGGCAAGCGGCCCGCCGCCACCAAGAAG |
| | | GCCGGCCAGGCCAAGAAGAAGAAGGGCAGCTACCCCTACGACGTGCCCGACTACGCCTGAGCGG |
| | | CCGCTTAATTAAGCTGCCTTCTGCGGGGCTTGCCTTCTGGCCATGCCCTTCTTCTCTCCCTTGC |
| | | ACCTGTACCTCTTGGTCTTTGAATAAAGCCTGAGTAGGAAGTCTAGAAAAAAAAAAAAAAAAAA |
| | | AAAAAAAAAAAAAAAAAAAAAAAAAAAAAAAAAAAAAAAAAAAAAAAAAAAAAAAAAAAAAA |
| MR- | EZH2- | AGGAAATAAGAGAGAAAAGAAGAGTAAGAAGAAATATAAGAGCCACCATGGCCCCCAAGAAGAA |
| 29949 | CXCL1 ZF1 | GCGGAAGGTGGGCGGCAGCGGCGGCAGCGGCCAGACCGGCAAGAAGAGCGAGAAGGGCCCCGTG |
| (SEQ ID | T7 | TGCTGGCGGAAGCGGGTGAAGAGCGAGTACATGCGGCTGCGGCAGCTGAAGCGGTTCCGGCGGG |
| NO: 220) | | CCGACGAGGTGAAGAGCATGTTCAGCAGCAACCGGCAGAAGATCCTGGAGCGGACCGAGATCCT |
| | | GAACCAGGAGTGGAAGCAGCGGCGAATCCAGCCCGTGCACATCCTGACCAGCGTGAGCAGCCTG |
| | | CGGGGCACCCGGGAGTGCAGCGTGACCAGCGACCTGGACTTCCCCACCCAGGTGATCCCCCTAA |
| | | AGACCCTGAACGCCGTGGCCAGCGTGCCCATCATGTACAGCTGGAGCCCCCTGCAGCAGAACTT |
| | | CATGGTGGAGGACGAGACCGTGCTGCACAACATCCCCTACATGGGCGACGAGGTGCTGGACCAG |
| | | GACGGCACCTTCATCGAGGAGCTGATCAAGAACTACGACGGCAAGGTGCACGGCGACCGGGAGT |
| | | GCGGCTTCATCAACGACGAGATCTTCGTGGAGCTGGTGAACGCCCTGGGCCAGTACAACGACGA |
| | | CGACGACGACGACGACGGCGACGACCCCGAGGAGCGGGAGGAGAAGCAGAAGGACCTGGAGGAC |
| | | CACCGGGACGACAAGGAGAGCCGGCCCCCCCGGAAGTTCCCCAGCGACAAGATCTTCGAGGCCA |
| | | TCAGCAGCATGTTCCCCGACAAGGGCACCGCCGAGGAGCTGAAGGAGAAGTACAAGGAGCTGAC |
| | | CGAGCAGCAGCTGCCCGGCGCCCTGCCCCCCGAGTGCACCCCCAACATCGACGGCCCCAACGCC |
| | | AAGAGCGTGCAGCGGGAGCAGAGCCTGCACAGCTTCCACACCCTGTTCTGCCGGCGGTGCTTCA |
| | | AGTACGACTGCTTCCTGCACCCCTTCCACGCCACCCCCAACACCTACAAGCGGAAGAACACCGA |
| | | GACCGCCCTGGACAACAAGCCCTGCGGCCCCCAGTGCTACCAGCACCTGGAGGGCGCCAAGGAG |
| | | TTCGCCGCCGCCCTGACCGCCGAGCGGATCAAGACCCCCCCCAAGCGGCCCGGCGGCCGGCGGC |
| | | GGGGCCGGCTGCCCAACAACAGCAGCCGGCCCAGCACCCCCACCATCAACGTGCTGGAGAGCAA |
| | | GGACACCGACAGCGACCGGGAGGCCGGCACCGAGACCGGCGGCGAGAACAACGACAAGGAGGAG |
| | | GAGGAGAAGAAGGACGAGACCAGCAGCAGCAGCGAGGCCAACAGCCGGTGCCAGACCCCCATCA |
| | | AGATGAAGCCCAACATCGAGCCCCCCGAGAACGTGGAGTGGAGCGGCGCCGAGGCCAGCATGTT |
| | | CCGGGTGCTGATCGGCACCTACTACGACAACTTCTGCGCCATCGCCCGGCTGATCGGCACCAAG |
| | | ACCTGCCGGCAGGTGTACGAGTTCCGGGTGAAGGAGAGCAGCATCATCGCCCCCGCCCCCGCCG |
| | | AGGACGTGGACACCCCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACCAGCCCTGCGACCAC |
| | | CCCCGGCAGCCCTGCGACAGCAGCTGCCCCTGCGTGATCGCCCAGAACTTCTGCGAGAAGTTCT |
| | | GCCAGTGCAGCAGCGAGTGCCAGAACCGGTTCCCCGGCTGCCGGTGCAAGGCCCAGTGCAACAC |
| | | CAAGCAGTGCCCCTGCTACCTGGCCGTGCGGGAGTGCGACCCCGACCTGTGCCTGACCTGCGGC |
| | | GCCGCCGACCACTGGGACAGCAAGAACGTGAGCTGCAAGAACTGCAGCATCCAGCGGGGCAGCA |
| | | AGAAGCACCTGCTGCTGGCCCCCAGCGACGTGGCCGGCTGGGGCATCTTCATCAAGGACCCCGT |
| | | GCAGAAGAACGAGTTCATCAGCGAGTACTGCGGCGAGATCATCAGCCAGGACGAGGCCGACCGG |
| | | CGGGGCAAGGTGTACGACAAGTACATGTGCAGCTTCCTGTTCAACCTGAACAACGACTTCGTGG |
| | | TGGACGCCACCCGGAAGGGCAACAAGATCCGGTTCGCCAACCACAGCGTGAACCCCAACTGCTA |
| | | CGCCAAGGTGATGATGGTGAACGGCGACCACCGGATCGGCATCTTCGCCAAGCGGGCCATCCAG |
| | | ACCGGCGAGGAGCTGTTCTTCGACTACCGGTACAGCCAGGCCGACGCCCTGAAGTACGTGGGCA |
| | | TCGAGCGGGAGATGGAGATCCCCGGCAGCAGCGGATCCCTGGAGCCCGGCGAAAAGCCTTACAA |
| | | ATGTCCCGAATGCGGAAAGAGCTTCAGCAGAGCCGACAATCTGACCGAACATCAGAGAACCCAT |
| | | ACCGGAGAAAAACCTTACAAATGTCCCGAGTGCGGCAAAAGCTTCTCCCAAGCCGGACATCTGG |
| | | CCAGCCACCAAAGGACACATACCGGCGAGAAACCCTACAAGTGCCCCGAGTGCGGCAAGTCCTT |
| | | CTCTAGATCCGATGAGCTGGTCAGACATCAGAGAACCCATACCGGCGAGAAGCCTTATAAGTGC |
| | | CCCGAATGTGGCAAGTCCTTCAGCCAGAGAGCTCATCTGGAGAGGCATCAAAGAACACACACCG |
| | | GAGAGAAACCTTACAAGTGTCCCGAGTGTGGAAAGAGCTTCTCCAGAAGGGACGAGCTGAACGT |
| | | CCACCAAAGAACCCATACCGGCGAAAAGCCCTATAAATGCCCCGAGTGTGGAAAATCCTTTTCT |
| | | AGATCCGACCATCTGACAACCCACCAGAGGACCCATACCGGAGAGAAGCCCTACAAATGCCCCG |
| | | AGTGTGGAAAAAGCTTCTCTAGAAACGATGCTCTGACAGAGCACCAAAGGACCCACACCGGCAA |
| | | AAAGACCAGCGCTAGCGGCAGCGGCGGCGGCAGCGGCGGCAAGCGGCCCGCCGCCACCAAGAAG |
| | | GCCGGCCAGGCCAAGAAGAAGAAGGGCAGCTACCCCTACGACGTGCCCGACTACGCCTGAGCGG |
| | | CCGCTTAATTAAGCTGCCTTCTGCGGGGCTTGCCTTCTGGCCATGCCCTTCTTCTCTCCCTTGC |
| | | ACCTGTACCTCTTGGTCTTTGAATAAAGCCTGAGTAGGAAGTCTAGAAAAAAAAAAAAAAAAAA |
| | | AAAAAAAAAAAAAAAAAAAAAAAAAAAAAAAAAAAAAAAAAAAAAAAAAAAAAAAAAAAAAA |
| MR- | CXCLI ZF5- | AGGAAATAAGAGAGAAAAGAAGAGTAAGAAGAAATATAAGAGCCACCATGGCCCCCAAGAAGAA |
| 29945 | DNMT3a-3L | GCGGAAGGTGGGCATCCACGGCGTGCCCGCCGCCGGCAGCAGCGGATCCCTGGAGCCCGGCGAA |
| (SEQ ID | SP6 | AAGCCTTACAAGTGCCCCGAGTGTGGCAAATCCTTTAGCACCACCGGAAATCTGACCGTCCACC |
| NO: 221) | | AGAGAACACATACCGGCGAGAAACCCTACAAGTGTCCCGAGTGCGGCAAATCCTTCAGCCAGCT |
| | | GGCCCATCTGAGAGCCCATCAAAGGACCCATACCGGCGAGAAACCTTACAAGTGTCCCGAATGT |
| | | GGAAAGTCCTTTAGCAGCCCCGCCGATCTGACAAGACATCAAAGAACCCACACCGGCGAGAAGC |
| | | CCTATAAATGTCCCGAGTGTGGAAAGTCCTTCAGCCAGAGCGGCAATCTGACCGAGCATCAAAG |
| | | AACCCATACCGGCGAAAAGCCCTATAAGTGCCCCGAATGCGGAAAAAGCTTCTCCACAAGCGGC |
| | | GAGCTGGTGAGACACCAAAGGACACATACCGGCGAAAAGCCTTATAAATGCCCCGAGTGCGGCA |
| | | AGAGCTTCTCTAGAAAGGACAATCTGAAGAACCACCAAAGAACACACACCGGCGAGAAGCCCTA |
| | | CAAATGCCCCGAGTGCGGCAAGAGCTTTAGCCAGTCCAGCAACCTCGTGAGACATCAGAGGACA |
| | | CATACCGGAAAAAAGACCAGCGCTAGCGGCAGCGGCGGCGGCAGCGGCGGCAACCACGACCAGG |
| | | AGTTCGACCCCCCCAAGGTGTACCCCCCCGTGCCCGCCGAGAAGCGGAAGCCCATCCGGGTGCT |
| | | GAGCCTGTTCGACGGCATCGCCACCGGCCTGCTGGTGCTGAAGGACCTGGGCATCCAGGTGGAC |
| | | CGGTACATCGCCAGCGAGGTGTGCGAGGACAGCATCACCGTGGGCATGGTGCGGCACCAGGGCA |
| | | AGATCATGTACGTGGGCGACGTGCGGAGCGTGACCCAGAAGCACATCCAGGAGTGGGGCCCCTT |
| | | CGACCTGGTGATCGGCGGCAGCCCCTGCAACGACCTGAGCATCGTGAACCCCGCCCGGAAGGGC |
| | | CTGTACGAGGGCACCGGCCGGCTGTTCTTCGAGTTCTACCGGCTGCTGCACGACGCCCGGCCCA |
| | | AGGAGGGCGACGACCGGCCCTTCTTCTGGCTGTTCGAGAACGTGGTGGCCATGGGCGTGAGCGA |
| | | CAAGCGGGACATCAGCCGGTTCCTGGAGAGCAACCCCGTGATGATCGACGCCAAGGAGGTGAGC |
| | | GCCGCCCACCGGGCCCGGTACTTCTGGGGCAACCTGCCCGGCATGAACCGGCCCCTGGCCAGCA |
| | | CCGTGAACGACAAGCTGGAGCTGCAGGAGTGCCTGGAGCACGGCCGGATCGCCAAGTTCAGCAA |
| | | GGTGCGGACCATCACCACCCGGAGCAACAGCATCAAGCAGGGCAAGGACCAGCACTTCCCCGTG |
| | | TTCATGAACGAGAAGGAGGACATCCTGTGGTGCACCGAGATGGAGCGGGTGTTCGGCTTCCCCG |
| | | TGCACTACACCGACGTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCGCCTGCGTGAGCAGC |
| | | GGCAACAGCAACGCCAACAGCCGGGGCCCCAGCTTCAGCAGCGGCCTGGTGCCCCTGAGCCTGC |
| | | GGGGCAGCCACATGAATCCTCTGGAGATGTTCGAGACAGTGCCCGTGTGGAGAAGGCAACCCGT |
| | | GAGGGTGCTGAGCCTCTTCGAGGACATTAAGAAGGAGCTGACCTCTCTGGGCTTTCTGGAATCC |
| | | GGCAGCGACCCCGGCCAGCTGAAACACGTGGTGGACGTGACCGACACAGTGAGGAAGGACGTGG |
| | | AAGAGTGGGGCCCCTTTGACCTCGTGTATGGAGCCACACCTCCTCTCGGCCACACATGCGATAG |
| | | GCCTCCCAGCTGGTATCTCTTCCAGTTCCACAGACTGCTCCAGTACGCCAGACCTAAGCCCGGC |
| | | AGCCCCAGACCCTTCTTCTGGATGTTCGTGGACAATCTGGTGCTGAACAAGGAGGATCTGGATG |
| | | TGGCCAGCAGATTTCTGGAGATGGAACCCGTGACAATCCCCGACGTGCATGGCGGCTCTCTGCA |
| | | GAACGCCGTGAGAGTGTGGTCCAACATCCCCGCCATTAGAAGCAGACACTGGGCTCTGGTGAGC |
| | | GAGGAGGAACTGTCTCTGCTGGCCCAGAATAAGCAGTCCTCCAAGCTGGCCGCCAAGTGGCCCA |
| | | CCAAGCTGGTGAAGAACTGCTTTCTGCCTCTGAGGGAGTATTTCAAGTATTTCAGCACCGAACT |
| | | GACCAGCAGCCTGAGCGGCGGCAAGCGGCCCGCCGCCACCAAGAAGGCCGGCCAGGCCAAGAAG |
| | | AAGAAGGGCAGCTACCCCTACGACGTGCCCGACTACGCCTGAGCGGCCGCTTAATTAAGCTGCC |
| | | TTCTGCGGGGCTTGCCTTCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAAAAAAAAAAAAAAAAAAA |
| | | AAAAAAAAAAAAAAAAAAAAAAAAAAAAAAAAAAAAAAAAAAAA |
| MR- | EZH2- | AGGAAATAAGAGAGAAAAGAAGAGTAAGAAGAAATATAAGAGCCACCATGGCCCCCAAGAAGAA |
| 29908 | CXCL1 ZF5 | GCGGAAGGTGGGCGGCAGCGGCGGCAGCGGCCAGACCGGCAAGAAGAGCGAGAAGGGCCCCGTG |
| (SEQ ID | T7 | TGCTGGCGGAAGCGGGTGAAGAGCGAGTACATGCGGCTGCGGCAGCTGAAGCGGTTCCGGCGGG |
| NO: 222) | | CCGACGAGGTGAAGAGCATGTTCAGCAGCAACCGGCAGAAGATCCTGGAGCGGACCGAGATCCT |
| | | GAACCAGGAGTGGAAGCAGCGGCGAATCCAGCCCGTGCACATCCTGACCAGCGTGAGCAGCCTG |
| | | CGGGGCACCCGGGAGTGCAGCGTGACCAGCGACCTGGACTTCCCCACCCAGGTGATCCCCCTAA |
| | | AGACCCTGAACGCCGTGGCCAGCGTGCCCATCATGTACAGCTGGAGCCCCCTGCAGCAGAACTT |
| | | CATGGTGGAGGACGAGACCGTGCTGCACAACATCCCCTACATGGGCGACGAGGTGCTGGACCAG |
| | | GACGGCACCTTCATCGAGGAGCTGATCAAGAACTACGACGGCAAGGTGCACGGCGACCGGGAGT |
| | | GCGGCTTCATCAACGACGAGATCTTCGTGGAGCTGGTGAACGCCCTGGGCCAGTACAACGACGA |
| | | CGACGACGACGACGACGGCGACGACCCCGAGGAGCGGGAGGAGAAGCAGAAGGACCTGGAGGAC |
| | | CACCGGGACGACAAGGAGAGCCGGCCCCCCCGGAAGTTCCCCAGCGACAAGATCTTCGAGGCCA |
| | | TCAGCAGCATGTTCCCCGACAAGGGCACCGCCGAGGAGCTGAAGGAGAAGTACAAGGAGCTGAC |
| | | CGAGCAGCAGCTGCCCGGCGCCCTGCCCCCCGAGTGCACCCCCAACATCGACGGCCCCAACGCC |
| | | AAGAGCGTGCAGCGGGAGCAGAGCCTGCACAGCTTCCACACCCTGTTCTGCCGGCGGTGCTTCA |
| | | AGTACGACTGCTTCCTGCACCCCTTCCACGCCACCCCCAACACCTACAAGCGGAAGAACACCGA |
| | | GACCGCCCTGGACAACAAGCCCTGCGGCCCCCAGTGCTACCAGCACCTGGAGGGCGCCAAGGAG |
| | | TTCGCCGCCGCCCTGACCGCCGAGCGGATCAAGACCCCCCCCAAGCGGCCCGGCGGCCGGCGGC |
| | | GGGGCCGGCTGCCCAACAACAGCAGCCGGCCCAGCACCCCCACCATCAACGTGCTGGAGAGCAA |
| | | GGACACCGACAGCGACCGGGAGGCCGGCACCGAGACCGGCGGCGAGAACAACGACAAGGAGGAG |
| | | GAGGAGAAGAAGGACGAGACCAGCAGCAGCAGCGAGGCCAACAGCCGGTGCCAGACCCCCATCA |
| | | AGATGAAGCCCAACATCGAGCCCCCCGAGAACGTGGAGTGGAGCGGCGCCGAGGCCAGCATGTT |
| | | CCGGGTGCTGATCGGCACCTACTACGACAACTTCTGCGCCATCGCCCGGCTGATCGGCACCAAG |
| | | ACCTGCCGGCAGGTGTACGAGTTCCGGGTGAAGGAGAGCAGCATCATCGCCCCCGCCCCCGCCG |
| | | AGGACGTGGACACCCCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACCAGCCCTGCGACCAC |
| | | CCCCGGCAGCCCTGCGACAGCAGCTGCCCCTGCGTGATCGCCCAGAACTTCTGCGAGAAGTTCT |
| | | GCCAGTGCAGCAGCGAGTGCCAGAACCGGTTCCCCGGCTGCCGGTGCAAGGCCCAGTGCAACAC |
| | | CAAGCAGTGCCCCTGCTACCTGGCCGTGCGGGAGTGCGACCCCGACCTGTGCCTGACCTGCGGC |
| | | GCCGCCGACCACTGGGACAGCAAGAACGTGAGCTGCAAGAACTGCAGCATCCAGCGGGGCAGCA |
| | | AGAAGCACCTGCTGCTGGCCCCCAGCGACGTGGCCGGCTGGGGCATCTTCATCAAGGACCCCGT |
| | | GCAGAAGAACGAGTTCATCAGCGAGTACTGCGGCGAGATCATCAGCCAGGACGAGGCCGACCGG |
| | | CGGGGCAAGGTGTACGACAAGTACATGTGCAGCTTCCTGTTCAACCTGAACAACGACTTCGTGG |
| | | TGGACGCCACCCGGAAGGGCAACAAGATCCGGTTCGCCAACCACAGCGTGAACCCCAACTGCTA |
| | | CGCCAAGGTGATGATGGTGAACGGCGACCACCGGATCGGCATCTTCGCCAAGCGGGCCATCCAG |
| | | ACCGGCGAGGAGCTGTTCTTCGACTACCGGTACAGCCAGGCCGACGCCCTGAAGTACGTGGGCA |
| | | TCGAGCGGGAGATGGAGATCCCCGGCAGCAGCGGATCCCTGGAGCCCGGCGAAAAGCCTTACAA |
| | | GTGCCCCGAGTGTGGCAAATCCTTTAGCACCACCGGAAATCTGACCGTCCACCAGAGAACACAT |
| | | ACCGGCGAGAAACCCTACAAGTGTCCCGAGTGCGGCAAATCCTTCAGCCAGCTGGCCCATCTGA |
| | | GAGCCCATCAAAGGACCCATACCGGCGAGAAACCTTACAAGTGTCCCGAATGTGGAAAGTCCTT |
| | | TAGCAGCCCCGCCGATCTGACAAGACATCAAAGAACCCACACCGGCGAGAAGCCCTATAAATGT |
| | | CCCGAGTGTGGAAAGTCCTTCAGCCAGAGCGGCAATCTGACCGAGCATCAAAGAACCCATACCG |
| | | GCGAAAAGCCCTATAAGTGCCCCGAATGCGGAAAAAGCTTCTCCACAAGCGGCGAGCTGGTGAG |
| | | ACACCAAAGGACACATACCGGCGAAAAGCCTTATAAATGCCCCGAGTGCGGCAAGAGCTTCTCT |
| | | AGAAAGGACAATCTGAAGAACCACCAAAGAACACACACCGGCGAGAAGCCCTACAAATGCCCCG |
| | | AGTGCGGCAAGAGCTTTAGCCAGTCCAGCAACCTCGTGAGACATCAGAGGACACATACCGGAAA |
| | | AAAGACCAGCGCTAGCGGCAGCGGCGGCGGCAGCGGCGGCAAGCGGCCCGCCGCCACCAAGAAG |
| | | GCCGGCCAGGCCAAGAAGAAGAAGGGCAGCTACCCCTACGACGTGCCCGACTACGCCTGAGCGG |
| | | CCGCTTAATTAAGCTGCCTTCTGCGGGGCTTGCCTTCTGGCCATGCCCTTCTTCTCTCCCTTGC |
| | | ACCTGTACCTCTTGGTCTTTGAATAAAGCCTGAGTAGGAAGTCTAGAAAAAAAAAAAAAAAAAA |
| | | AAAAAAAAAAAAAAAAAAAAAAAAAAAAAAAAAAAAAAAAAAAAAAAAAAAAAAAAAAAAAA |
| MR- | EZH2- | AGGAAATAAGAGAGAAAAGAAGAGTAAGAAGAAATATAAGAGCCACCATGGCCCCCAAGAAGAA |
| 29907 | CXCL1 ZF2 | GCGGAAGGTGGGCGGCAGCGGCGGCAGCGGCCAGACCGGCAAGAAGAGCGAGAAGGGCCCCGTG |
| (SEQ ID | T7 | TGCTGGCGGAAGCGGGTGAAGAGCGAGTACATGCGGCTGCGGCAGCTGAAGCGGTTCCGGCGGG |
| NO: 223) | | CCGACGAGGTGAAGAGCATGTTCAGCAGCAACCGGCAGAAGATCCTGGAGCGGACCGAGATCCT |
| | | GAACCAGGAGTGGAAGCAGCGGCGAATCCAGCCCGTGCACATCCTGACCAGCGTGAGCAGCCTG |
| | | CGGGGCACCCGGGAGTGCAGCGTGACCAGCGACCTGGACTTCCCCACCCAGGTGATCCCCCTAA |
| | | AGACCCTGAACGCCGTGGCCAGCGTGCCCATCATGTACAGCTGGAGCCCCCTGCAGCAGAACTT |
| | | CATGGTGGAGGACGAGACCGTGCTGCACAACATCCCCTACATGGGCGACGAGGTGCTGGACCAG |
| | | GACGGCACCTTCATCGAGGAGCTGATCAAGAACTACGACGGCAAGGTGCACGGCGACCGGGAGT |
| | | GCGGCTTCATCAACGACGAGATCTTCGTGGAGCTGGTGAACGCCCTGGGCCAGTACAACGACGA |
| | | CGACGACGACGACGACGGCGACGACCCCGAGGAGCGGGAGGAGAAGCAGAAGGACCTGGAGGAC |
| | | CACCGGGACGACAAGGAGAGCCGGCCCCCCCGGAAGTTCCCCAGCGACAAGATCTTCGAGGCCA |
| | | TCAGCAGCATGTTCCCCGACAAGGGCACCGCCGAGGAGCTGAAGGAGAAGTACAAGGAGCTGAC |
| | | CGAGCAGCAGCTGCCCGGCGCCCTGCCCCCCGAGTGCACCCCCAACATCGACGGCCCCAACGCC |
| | | AAGAGCGTGCAGCGGGAGCAGAGCCTGCACAGCTTCCACACCCTGTTCTGCCGGCGGTGCTTCA |
| | | AGTACGACTGCTTCCTGCACCCCTTCCACGCCACCCCCAACACCTACAAGCGGAAGAACACCGA |
| | | GACCGCCCTGGACAACAAGCCCTGCGGCCCCCAGTGCTACCAGCACCTGGAGGGCGCCAAGGAG |
| | | TTCGCCGCCGCCCTGACCGCCGAGCGGATCAAGACCCCCCCCAAGCGGCCCGGCGGCCGGCGGC |
| | | GGGGCCGGCTGCCCAACAACAGCAGCCGGCCCAGCACCCCCACCATCAACGTGCTGGAGAGCAA |
| | | GGACACCGACAGCGACCGGGAGGCCGGCACCGAGACCGGCGGCGAGAACAACGACAAGGAGGAG |
| | | GAGGAGAAGAAGGACGAGACCAGCAGCAGCAGCGAGGCCAACAGCCGGTGCCAGACCCCCATCA |
| | | AGATGAAGCCCAACATCGAGCCCCCCGAGAACGTGGAGTGGAGCGGCGCCGAGGCCAGCATGTT |
| | | CCGGGTGCTGATCGGCACCTACTACGACAACTTCTGCGCCATCGCCCGGCTGATCGGCACCAAG |
| | | ACCTGCCGGCAGGTGTACGAGTTCCGGGTGAAGGAGAGCAGCATCATCGCCCCCGCCCCCGCCG |
| | | AGGACGTGGACACCCCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACCAGCCCTGCGACCAC |
| | | CCCCGGCAGCCCTGCGACAGCAGCTGCCCCTGCGTGATCGCCCAGAACTTCTGCGAGAAGTTCT |
| | | GCCAGTGCAGCAGCGAGTGCCAGAACCGGTTCCCCGGCTGCCGGTGCAAGGCCCAGTGCAACAC |
| | | CAAGCAGTGCCCCTGCTACCTGGCCGTGCGGGAGTGCGACCCCGACCTGTGCCTGACCTGCGGC |
| | | GCCGCCGACCACTGGGACAGCAAGAACGTGAGCTGCAAGAACTGCAGCATCCAGCGGGGCAGCA |
| | | AGAAGCACCTGCTGCTGGCCCCCAGCGACGTGGCCGGCTGGGGCATCTTCATCAAGGACCCCGT |
| | | GCAGAAGAACGAGTTCATCAGCGAGTACTGCGGCGAGATCATCAGCCAGGACGAGGCCGACCGG |
| | | CGGGGCAAGGTGTACGACAAGTACATGTGCAGCTTCCTGTTCAACCTGAACAACGACTTCGTGG |
| | | TGGACGCCACCCGGAAGGGCAACAAGATCCGGTTCGCCAACCACAGCGTGAACCCCAACTGCTA |
| | | CGCCAAGGTGATGATGGTGAACGGCGACCACCGGATCGGCATCTTCGCCAAGCGGGCCATCCAG |
| | | ACCGGCGAGGAGCTGTTCTTCGACTACCGGTACAGCCAGGCCGACGCCCTGAAGTACGTGGGCA |
| | | TCGAGCGGGAGATGGAGATCCCCGGCAGCAGCGGATCCCTGGAGCCCGGCGAAAAACCCTATAA |
| | | GTGCCCCGAGTGCGGCAAGAGCTTTAGCGATCCCGGCCATCTGGTGAGGCATCAGAGGACCCAC |
| | | ACCGGCGAAAAGCCTTACAAATGCCCCGAGTGTGGAAAAAGCTTCAGCAGAAGCGATCATCTGA |
| | | CCACCCATCAGAGGACACATACCGGCGAGAAGCCTTATAAATGCCCCGAATGTGGAAAGAGCTT |
| | | CTCCAGAAGCGACCATCTGACCAACCACCAGAGGACCCATACCGGAGAAAAACCTTACAAATGC |
| | | CCCGAGTGTGGAAAGTCCTTCAGCTCCCCCGCCGATCTGACAAGACATCAGAGAACCCACACCG |
| | | GCGAAAAACCTTATAAATGTCCCGAGTGTGGCAAAAGCTTCTCCGACAAGAAGGATCTGACAAG |
| | | ACACCAAAGGACCCACACCGGCGAGAAACCTTATAAATGTCCCGAATGCGGAAAAAGCTTTAGC |
| | | AGAAACGACGCTCTGACCGAACACCAGAGAACACATACCGGAGAGAAACCCTATAAATGTCCCG |
| | | AGTGCGGAAAATCCTTCAGCACCACCGGCGCTCTGACAGAGCATCAGAGGACACACACCGGCAA |
| | | AAAGACCTCCGCTAGCGGCAGCGGCGGCGGCAGCGGCGGCAAGCGGCCCGCCGCCACCAAGAAG |
| | | GCCGGCCAGGCCAAGAAGAAGAAGGGCAGCTACCCCTACGACGTGCCCGACTACGCCTGAGCGG |
| | | CCGCTTAATTAAGCTGCCTTCTGCGGGGCTTGCCTTCTGGCCATGCCCTTCTTCTCTCCCTTGC |
| | | ACCTGTACCTCTTGGTCTTTGAATAAAGCCTGAGTAGGAAGTCTAGAAAAAAAAAAAAAAAAAA |
| | | AAAAAAAAAAAAAAAAAAAAAAAAAAAAAAAAAAAAAAAAAAAAAAAAAAAAAAAAAAAAAA |
| MR- | G9A-CXCL1 | AGGAAATAAGAGAGAAAAGAAGAGTAAGAAGAAATATAAGAGCCACCATGGCCCCCAAGAAGAA |
| 29906 | ZF4 SP6 | GCGGAAGGTGGGCATCCACGGCGTGCCCGCCGCCTCGGGCGGGGGTGGCTCAGGAAATAGGGCT |
| (SEQ ID | | ATCAGAACCGAGAAGATCATCTGTAGGGACGTGGCTAGAGGCTACGAGAACGTGCCCATTCCTT |
| NO: 224) | | GCGTGAATGGCGTGGATGGCGAACCTTGCCCCGAGGACTACAAATACATCTCCGAGAACTGCGA |
| | | AACCAGCACAATGAACATCGACAGAAACATCACCCACCTCCAGCACTGCACATGTGTGGATGAC |
| | | TGCTCCTCCAGCAACTGTCTGTGCGGCCAGCTCTCCATCAGATGCTGGTACGACAAGGACGGCA |
| | | GACTGCTGCAAGAGTTCAACAAGATCGAACCCCCTCTCATCTTCGAGTGTAACCAAGCTTGCAG |
| | | CTGCTGGAGAAACTGCAAGAATAGAGTGGTCCAGAGCGGCATCAAGGTGAGACTGCAACTGTAC |
| | | AGAACCGCCAAGATGGGATGGGGAGTGAGGGCTCTGCAAACCATTCCCCAAGGCACCTTCATCT |
| | | GCGAATACGTGGGCGAACTGATCTCCGACGCCGAAGCTGACGTGAGAGAGGACGACAGCTATCT |
| | | CTTCGATCTGGACAATAAGGACGGCGAGGTGTACTGCATCGACGCTAGATATTACGGCAACATC |
| | | TCTAGATTCATCAACCACCTCTGCGATCCCAACATCATTCCCGTGAGGGTGTTCATGCTGCACC |
| | | AAGATCTGAGGTTCCCTAGAATCGCCTTCTTCAGCTCTAGAGACATCAGAACCGGCGAGGAGCT |
| | | GGGCTTCGATTACGGCGATAGATTCTGGGACATCAAGTCCAAGTACTTCACATGCCAGTGCGGC |
| | | AGCGAGAAGTGTAAGCACAGCGCTGAGGCCATTGCTCTGGAGCAGTCTAGACTGGCCAGACTGG |
| | | ATGGCAGCAGCGGATCCCTGGAGCCCGGAGAAAAGCCTTACAAATGCCCCGAGTGCGGCAAGTC |
| | | CTTCAGCCAGCTGGCTCATCTGAGAGCTCATCAAAGGACCCACACCGGCGAGAAGCCCTATAAG |
| | | TGCCCCGAGTGCGGAAAATCCTTCTCCCAGAGCAGCAATCTCGTCAGACACCAGAGGACCCACA |
| | | CCGGCGAGAAACCTTACAAGTGTCCCGAATGTGGAAAGTCCTTCTCCCAAAAGAGCTCTCTGAT |
| | | CGCCCATCAGAGAACACATACCGGCGAAAAACCCTACAAGTGCCCCGAGTGTGGCAAAAGCTTT |
| | | TCCACCACCGGCAATCTGACCGTGCATCAAAGAACCCACACCGGCGAAAAACCCTACAAATGCC |
| | | CCGAGTGTGGCAAATCCTTCTCCGACCCCGGCCATCTGGTGAGGCACCAGAGGACACACACCGG |
| | | CGAGAAACCTTATAAATGTCCCGAATGCGGCAAGTCCTTTAGCACCAGCGGCTCTCTGGTGAGA |
| | | CATCAGAGGACACATACCGGCGAAAAGCCTTACAAGTGTCCCGAGTGTGGCAAAAGCTTCAGCC |
| | | AGAACAGCACACTGACAGAGCATCAGAGAACCCATACCGGCAAAAAGACCAGCGCTAGCGGCAG |
| | | CGGCGGCGGCAGCGGCGGCAAGCGGCCCGCCGCCACCAAGAAGGCCGGCCAGGCCAAGAAGAAG |
| | | AAGGGCAGCTACCCCTACGACGTGCCCGACTACGCCTGAGCGGCCGCTTAATTAAGCTGCCTTC |
| | | TGCGGGGCTTGCCTTCTGGCCATGCCCTTCTTCTCTCCCTTGCACCTGTACCTCTTGGTCTTTG |
| | | AATAAAGCCTGAGTAGGAAGTCTAGAAAAAAAAAAAAAAAAAAAAAAAAAAAAAAAAAAAAAAA |
| | | AAAAAAAAAAAAAAAAAAAAAAAAAAAAAAAAAAAAAAAAA |
| MR- | G9A-CXCLI | AGGAAATAAGAGAGAAAAGAAGAGTAAGAAGAAATATAAGAGCCACCATGGCCCCCAAGAAGAA |
| 29904 | ZF5 SP6 | GCGGAAGGTGGGCATCCACGGCGTGCCCGCCGCCTCGGGCGGGGGTGGCTCAGGAAATAGGGCT |
| (SEQ ID | | ATCAGAACCGAGAAGATCATCTGTAGGGACGTGGCTAGAGGCTACGAGAACGTGCCCATTCCTT |
| NO: 225) | | GCGTGAATGGCGTGGATGGCGAACCTTGCCCCGAGGACTACAAATACATCTCCGAGAACTGCGA |
| | | AACCAGCACAATGAACATCGACAGAAACATCACCCACCTCCAGCACTGCACATGTGTGGATGAC |
| | | TGCTCCTCCAGCAACTGTCTGTGCGGCCAGCTCTCCATCAGATGCTGGTACGACAAGGACGGCA |
| | | GACTGCTGCAAGAGTTCAACAAGATCGAACCCCCTCTCATCTTCGAGTGTAACCAAGCTTGCAG |
| | | CTGCTGGAGAAACTGCAAGAATAGAGTGGTCCAGAGCGGCATCAAGGTGAGACTGCAACTGTAC |
| | | AGAACCGCCAAGATGGGATGGGGAGTGAGGGCTCTGCAAACCATTCCCCAAGGCACCTTCATCT |
| | | GCGAATACGTGGGCGAACTGATCTCCGACGCCGAAGCTGACGTGAGAGAGGACGACAGCTATCT |
| | | CTTCGATCTGGACAATAAGGACGGCGAGGTGTACTGCATCGACGCTAGATATTACGGCAACATC |
| | | TCTAGATTCATCAACCACCTCTGCGATCCCAACATCATTCCCGTGAGGGTGTTCATGCTGCACC |
| | | AAGATCTGAGGTTCCCTAGAATCGCCTTCTTCAGCTCTAGAGACATCAGAACCGGCGAGGAGCT |
| | | GGGCTTCGATTACGGCGATAGATTCTGGGACATCAAGTCCAAGTACTTCACATGCCAGTGCGGC |
| | | AGCGAGAAGTGTAAGCACAGCGCTGAGGCCATTGCTCTGGAGCAGTCTAGACTGGCCAGACTGG |
| | | ATGGCAGCAGCGGATCCCTGGAGCCCGGCGAAAAGCCTTACAAGTGCCCCGAGTGTGGCAAATC |
| | | CTTTAGCACCACCGGAAATCTGACCGTCCACCAGAGAACACATACCGGCGAGAAACCCTACAAG |
| | | TGTCCCGAGTGCGGCAAATCCTTCAGCCAGCTGGCCCATCTGAGAGCCCATCAAAGGACCCATA |
| | | CCGGCGAGAAACCTTACAAGTGTCCCGAATGTGGAAAGTCCTTTAGCAGCCCCGCCGATCTGAC |
| | | AAGACATCAAAGAACCCACACCGGCGAGAAGCCCTATAAATGTCCCGAGTGTGGAAAGTCCTTC |
| | | AGCCAGAGCGGCAATCTGACCGAGCATCAAAGAACCCATACCGGCGAAAAGCCCTATAAGTGCC |
| | | CCGAATGCGGAAAAAGCTTCTCCACAAGCGGCGAGCTGGTGAGACACCAAAGGACACATACCGG |
| | | CGAAAAGCCTTATAAATGCCCCGAGTGCGGCAAGAGCTTCTCTAGAAAGGACAATCTGAAGAAC |
| | | CACCAAAGAACACACACCGGCGAGAAGCCCTACAAATGCCCCGAGTGCGGCAAGAGCTTTAGCC |
| | | AGTCCAGCAACCTCGTGAGACATCAGAGGACACATACCGGAAAAAAGACCAGCGCTAGCGGCAG |
| | | CGGCGGCGGCAGCGGCGGCAAGCGGCCCGCCGCCACCAAGAAGGCCGGCCAGGCCAAGAAGAAG |
| | | AAGGGCAGCTACCCCTACGACGTGCCCGACTACGCCTGAGCGGCCGCTTAATTAAGCTGCCTTC |
| | | TGCGGGGCTTGCCTTCTGGCCATGCCCTTCTTCTCTCCCTTGCACCTGTACCTCTTGGTCTTTG |
| | | AATAAAGCCTGAGTAGGAAGTCTAGAAAAAAAAAAAAAAAAAAAAAAAAAAAAAAAAAAAAAAA |
| | | AAAAAAAAAAAAAAAAAAAAAAAAAAAAAAAAAAAAAAAAA |
| MR- | CXCL1 ZF4- | AGGAAATAAGAGAGAAAAGAAGAGTAAGAAGAAATATAAGAGCCACCATGGCCCCCAAGAAGAA |
| 29890 | HDAC8 SP6 | GCGGAAGGTGGGCATCCACGGCGTGCCCGCCGCCGGCAGCAGCGGATCCCTGGAGCCCGGAGAA |
| (SEQ ID | | AAGCCTTACAAATGCCCCGAGTGCGGCAAGTCCTTCAGCCAGCTGGCTCATCTGAGAGCTCATC |
| NO: 226) | | AAAGGACCCACACCGGCGAGAAGCCCTATAAGTGCCCCGAGTGCGGAAAATCCTTCTCCCAGAG |
| | | CAGCAATCTCGTCAGACACCAGAGGACCCACACCGGCGAGAAACCTTACAAGTGTCCCGAATGT |
| | | GGAAAGTCCTTCTCCCAAAAGAGCTCTCTGATCGCCCATCAGAGAACACATACCGGCGAAAAAC |
| | | CCTACAAGTGCCCCGAGTGTGGCAAAAGCTTTTCCACCACCGGCAATCTGACCGTGCATCAAAG |
| | | AACCCACACCGGCGAAAAACCCTACAAATGCCCCGAGTGTGGCAAATCCTTCTCCGACCCCGGC |
| | | CATCTGGTGAGGCACCAGAGGACACACACCGGCGAGAAACCTTATAAATGTCCCGAATGCGGCA |
| | | AGTCCTTTAGCACCAGCGGCTCTCTGGTGAGACATCAGAGGACACATACCGGCGAAAAGCCTTA |
| | | CAAGTGTCCCGAGTGTGGCAAAAGCTTCAGCCAGAACAGCACACTGACAGAGCATCAGAGAACC |
| | | CATACCGGCAAAAAGACCAGCGCTAGCGGCAGCGGCGGCGGCAGCGGCGGCGAGGAGCCCGAGG |
| | | AGCCCGCCGATAGCGGACAATCTCTGGTGCCCGTCTACATCTACAGCCCCGAATATGTGAGCAT |
| | | GTGTGATTCCCTCGCCAAGATCCCTAAGAGAGCCAGCATGGTGCATTCTCTGATCGAGGCCTAC |
| | | GCTCTGCATAAGCAAATGAGGATCGTGAAGCCCAAGGTCGCCAGCATGGAAGAGATGGCCACCT |
| | | TTCACACCGATGCCTACCTCCAACATCTCCAGAAGGTGTCCCAAGAGGGCGACGACGACCACCC |
| | | CGACTCCATTGAGTACGGACTGGGCTATGATTGCCCCGCCACCGAGGGCATCTTTGACTATGCC |
| | | GCCGCTATCGGCGGAGCTACCATCACAGCCGCCCAGTGTCTGATTGATGGCATGTGCAAGGTCG |
| | | CCATCAACTGGTCCGGAGGCTGGCATCATGCCAAGAAGGATGAGGCCTCCGGCTTCTGTTATCT |
| | | GAATGACGCCGTGCTGGGCATTCTGAGACTGAGGAGGAAATTCGAGAGGATTCTGTACGTGGAT |
| | | CTGGATCTGCATCACGGAGATGGAGTCGAAGATGCCTTCAGCTTCACCAGCAAGGTGATGACAG |
| | | TCTCTCTGCACAAGTTCTCCCCCGGCTTCTTTCCCGGAACCGGCGACGTGTCCGACGTGGGACT |
| | | GGGCAAGGGAAGGTACTACAGCGTGAACGTGCCCATTCAAGACGGCATCCAAGACGAGAAGTAC |
| | | TACCAGATCTGCGAGTCCGTGCTCAAGGAGGTCTACCAAGCCTTCAATCCTAAGGCTGTCGTGC |
| | | TCCAACTGGGAGCTGATACCATTGCTGGCGATCCCATGTGCAGCTTCAATATGACACCCGTCGG |
| | | AATCGGCAAGTGCCTCAAGTACATCCTCCAGTGGCAGCTCGCCACCCTCATTCTCGGAGGAGGC |
| | | GGATACAATCTGGCTAATACCGCCAGATGCTGGACCTATCTGACCGGCGTGATTCTGGGCAAAA |
| | | CACTGAGCAGCGAAATCCCCGACCACGAGTTTTTCACCGCTTACGGCCCCGACTACGTGCTGGA |
| | | GATCACCCCCAGCTGCAGACCCGATAGAAACGAACCCCATAGAATCCAGCAAATTCTGAACTAT |
| | | ATCAAGGGCAACCTCAAGCACGTCGTGGGAGGTGGCGGATCGGGAAAGCGGCCCGCCGCCACCA |
| | | AGAAGGCCGGTCAGGCCAAGAAGAAGAAGGGCAGCTACCCCTACGACGTGCCCGACTACGCCTG |
| | | AGCGGCCGCTTAATTAAGCTGCCTTCTGCGGGGCTTGCCTTCTGGCCATGCCCTTCTTCTCTCC |
| | | CTTGCACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAAAAAA |
| | | AAA |
| MR- | G9A-CXCL1 | AGGAAATAAGAGAGAAAAGAAGAGTAAGAAGAAATATAAGAGCCACCATGGCCCCCAAGAAGAA |
| 29886 | ZF6 SP6 | GCGGAAGGTGGGCATCCACGGCGTGCCCGCCGCCTCGGGCGGGGGTGGCTCAGGAAATAGGGCT |
| (SEQ ID | | ATCAGAACCGAGAAGATCATCTGTAGGGACGTGGCTAGAGGCTACGAGAACGTGCCCATTCCTT |
| NO: 227) | | GCGTGAATGGCGTGGATGGCGAACCTTGCCCCGAGGACTACAAATACATCTCCGAGAACTGCGA |
| | | AACCAGCACAATGAACATCGACAGAAACATCACCCACCTCCAGCACTGCACATGTGTGGATGAC |
| | | TGCTCCTCCAGCAACTGTCTGTGCGGCCAGCTCTCCATCAGATGCTGGTACGACAAGGACGGCA |
| | | GACTGCTGCAAGAGTTCAACAAGATCGAACCCCCTCTCATCTTCGAGTGTAACCAAGCTTGCAG |
| | | CTGCTGGAGAAACTGCAAGAATAGAGTGGTCCAGAGCGGCATCAAGGTGAGACTGCAACTGTAC |
| | | AGAACCGCCAAGATGGGATGGGGAGTGAGGGCTCTGCAAACCATTCCCCAAGGCACCTTCATCT |
| | | GCGAATACGTGGGCGAACTGATCTCCGACGCCGAAGCTGACGTGAGAGAGGACGACAGCTATCT |
| | | CTTCGATCTGGACAATAAGGACGGCGAGGTGTACTGCATCGACGCTAGATATTACGGCAACATC |
| | | TCTAGATTCATCAACCACCTCTGCGATCCCAACATCATTCCCGTGAGGGTGTTCATGCTGCACC |
| | | AAGATCTGAGGTTCCCTAGAATCGCCTTCTTCAGCTCTAGAGACATCAGAACCGGCGAGGAGCT |
| | | GGGCTTCGATTACGGCGATAGATTCTGGGACATCAAGTCCAAGTACTTCACATGCCAGTGCGGC |
| | | AGCGAGAAGTGTAAGCACAGCGCTGAGGCCATTGCTCTGGAGCAGTCTAGACTGGCCAGACTGG |
| | | ATGGCAGCAGCGGATCCCTGGAGCCCGGCGAAAAGCCTTACAAGTGTCCCGAGTGCGGAAAGAG |
| | | CTTCAGCAGAGCCGATAATCTGACCGAGCACCAAAGGACCCACACCGGAGAGAAGCCTTATAAG |
| | | TGTCCCGAATGCGGCAAAAGCTTTTCTAGAAGCGATCATCTGACCAACCACCAGAGGACACACA |
| | | CCGGAGAAAAACCTTACAAATGCCCCGAGTGCGGCAAAAGCTTCTCCCAGAGCAGCAATCTGGT |
| | | GAGACACCAAAGGACCCACACCGGCGAAAAACCCTATAAATGCCCCGAATGTGGCAAGAGCTTT |
| | | AGCACATCCGGCGAGCTGGTGAGGCATCAAAGAACACATACCGGCGAGAAGCCCTACAAGTGCC |
| | | CCGAGTGTGGAAAAAGCTTCAGCACCCACCTCGATCTGATCAGACACCAGAGGACCCATACCGG |
| | | AGAGAAACCCTACAAATGTCCCGAGTGCGGAAAGTCCTTTAGCCAGCTGGCCCATCTGAGAGCT |
| | | CATCAAAGGACACACACCGGCGAGAAGCCTTACAAGTGTCCCGAGTGCGGAAAATCCTTCTCCC |
| | | AACTGGCCCATCTGAGGGCCCACCAGAGAACCCACACCGGCAAAAAGACCTCCGCTAGCGGCAG |
| | | CGGCGGCGGCAGCGGCGGCAAGCGGCCCGCCGCCACCAAGAAGGCCGGCCAGGCCAAGAAGAAG |
| | | AAGGGCAGCTACCCCTACGACGTGCCCGACTACGCCTGAGCGGCCGCTTAATTAAGCTGCCTTC |
| | | TGCGGGGCTTGCCTTCTGGCCATGCCCTTCTTCTCTCCCTTGCACCTGTACCTCTTGGTCTTTG |
| | | AATAAAGCCTGAGTAGGAAGTCTAGAAAAAAAAAAAAAAAAAAAAAAAAAAAAAAAAAAAAAAA |
| | | AAAAAAAAAAAAAAAAAAAAAAAAAAAAAAAAAAAAAAAAA |
| MR- | G9A-CXCL1 | AGGAAATAAGAGAGAAAAGAAGAGTAAGAAGAAATATAAGAGCCACCATGGCCCCCAAGAAGAA |
| 29873 | ZF3 SP6 | GCGGAAGGTGGGCATCCACGGCGTGCCCGCCGCCTCGGGCGGGGGTGGCTCAGGAAATAGGGCT |
| (SEQ ID | | ATCAGAACCGAGAAGATCATCTGTAGGGACGTGGCTAGAGGCTACGAGAACGTGCCCATTCCTT |
| NO: 228) | | GCGTGAATGGCGTGGATGGCGAACCTTGCCCCGAGGACTACAAATACATCTCCGAGAACTGCGA |
| | | AACCAGCACAATGAACATCGACAGAAACATCACCCACCTCCAGCACTGCACATGTGTGGATGAC |
| | | TGCTCCTCCAGCAACTGTCTGTGCGGCCAGCTCTCCATCAGATGCTGGTACGACAAGGACGGCA |
| | | GACTGCTGCAAGAGTTCAACAAGATCGAACCCCCTCTCATCTTCGAGTGTAACCAAGCTTGCAG |
| | | CTGCTGGAGAAACTGCAAGAATAGAGTGGTCCAGAGCGGCATCAAGGTGAGACTGCAACTGTAC |
| | | AGAACCGCCAAGATGGGATGGGGAGTGAGGGCTCTGCAAACCATTCCCCAAGGCACCTTCATCT |
| | | GCGAATACGTGGGCGAACTGATCTCCGACGCCGAAGCTGACGTGAGAGAGGACGACAGCTATCT |
| | | CTTCGATCTGGACAATAAGGACGGCGAGGTGTACTGCATCGACGCTAGATATTACGGCAACATC |
| | | TCTAGATTCATCAACCACCTCTGCGATCCCAACATCATTCCCGTGAGGGTGTTCATGCTGCACC |
| | | AAGATCTGAGGTTCCCTAGAATCGCCTTCTTCAGCTCTAGAGACATCAGAACCGGCGAGGAGCT |
| | | GGGCTTCGATTACGGCGATAGATTCTGGGACATCAAGTCCAAGTACTTCACATGCCAGTGCGGC |
| | | AGCGAGAAGTGTAAGCACAGCGCTGAGGCCATTGCTCTGGAGCAGTCTAGACTGGCCAGACTGG |
| | | ATGGCAGCAGCGGATCCCTGGAGCCCGGCGAAAAACCCTATAAATGCCCCGAGTGTGGCAAGAG |
| | | CTTTTCCGACCCCGGACACCTCGTGAGGCATCAGAGAACACATACCGGCGAGAAACCCTACAAG |
| | | TGCCCCGAATGCGGCAAATCCTTCTCTAGAAAGGACAATCTGAAAAACCATCAAAGAACCCATA |
| | | CCGGCGAGAAGCCCTATAAATGTCCCGAGTGTGGAAAGAGCTTCAGCCACAAGAACGCTCTGCA |
| | | GAACCATCAGAGGACCCATACCGGCGAAAAGCCTTATAAGTGCCCCGAGTGCGGAAAATCCTTT |
| | | TCTAGAAGGGACGAGCTGAATGTGCACCAAAGGACACATACCGGAGAGAAACCCTACAAATGCC |
| | | CCGAGTGCGGCAAGTCCTTCAGCACCTCCGGCAATCTGGTGAGGCACCAAAGGACACACACCGG |
| | | CGAAAAACCTTACAAGTGTCCCGAGTGCGGAAAAAGCTTTTCCCAGAACAGCACACTGACCGAA |
| | | CACCAAAGGACCCACACCGGAGAGAAACCTTATAAATGTCCCGAGTGTGGAAAGTCCTTTAGCC |
| | | AGTCCGGCAATCTGACAGAGCATCAAAGAACCCACACCGGCAAAAAGACCTCCGCTAGCGGCAG |
| | | CGGCGGCGGCAGCGGCGGCAAGCGGCCCGCCGCCACCAAGAAGGCCGGCCAGGCCAAGAAGAAG |
| | | AAGGGCAGCTACCCCTACGACGTGCCCGACTACGCCTGAGCGGCCGCTTAATTAAGCTGCCTTC |
| | | TGCGGGGCTTGCCTTCTGGCCATGCCCTTCTTCTCTCCCTTGCACCTGTACCTCTTGGTCTTTG |
| | | AATAAAGCCTGAGTAGGAAGTCTAGAAAAAAAAAAAAAAAAAAAAAAAAAAAAAAAAAAAAAAA |
| | | AAAAAAAAAAAAAAAAAAAAAAAAAAAAAAAAAAAAAAAAA |
| MR- | G9A-CXCL1 | AGGAAATAAGAGAGAAAAGAAGAGTAAGAAGAAATATAAGAGCCACCATGGCCCCCAAGAAGAA |
| 29872 | ZF2 SP6 | GCGGAAGGTGGGCATCCACGGCGTGCCCGCCGCCTCGGGCGGGGGTGGCTCAGGAAATAGGGCT |
| (SEQ ID | | ATCAGAACCGAGAAGATCATCTGTAGGGACGTGGCTAGAGGCTACGAGAACGTGCCCATTCCTT |
| NO: 229) | | GCGTGAATGGCGTGGATGGCGAACCTTGCCCCGAGGACTACAAATACATCTCCGAGAACTGCGA |
| | | AACCAGCACAATGAACATCGACAGAAACATCACCCACCTCCAGCACTGCACATGTGTGGATGAC |
| | | TGCTCCTCCAGCAACTGTCTGTGCGGCCAGCTCTCCATCAGATGCTGGTACGACAAGGACGGCA |
| | | GACTGCTGCAAGAGTTCAACAAGATCGAACCCCCTCTCATCTTCGAGTGTAACCAAGCTTGCAG |
| | | CTGCTGGAGAAACTGCAAGAATAGAGTGGTCCAGAGCGGCATCAAGGTGAGACTGCAACTGTAC |
| | | AGAACCGCCAAGATGGGATGGGGAGTGAGGGCTCTGCAAACCATTCCCCAAGGCACCTTCATCT |
| | | GCGAATACGTGGGCGAACTGATCTCCGACGCCGAAGCTGACGTGAGAGAGGACGACAGCTATCT |
| | | CTTCGATCTGGACAATAAGGACGGCGAGGTGTACTGCATCGACGCTAGATATTACGGCAACATC |
| | | TCTAGATTCATCAACCACCTCTGCGATCCCAACATCATTCCCGTGAGGGTGTTCATGCTGCACC |
| | | AAGATCTGAGGTTCCCTAGAATCGCCTTCTTCAGCTCTAGAGACATCAGAACCGGCGAGGAGCT |
| | | GGGCTTCGATTACGGCGATAGATTCTGGGACATCAAGTCCAAGTACTTCACATGCCAGTGCGGC |
| | | AGCGAGAAGTGTAAGCACAGCGCTGAGGCCATTGCTCTGGAGCAGTCTAGACTGGCCAGACTGG |
| | | ATGGCAGCAGCGGATCCCTGGAGCCCGGCGAAAAACCCTATAAGTGCCCCGAGTGCGGCAAGAG |
| | | CTTTAGCGATCCCGGCCATCTGGTGAGGCATCAGAGGACCCACACCGGCGAAAAGCCTTACAAA |
| | | TGCCCCGAGTGTGGAAAAAGCTTCAGCAGAAGCGATCATCTGACCACCCATCAGAGGACACATA |
| | | CCGGCGAGAAGCCTTATAAATGCCCCGAATGTGGAAAGAGCTTCTCCAGAAGCGACCATCTGAC |
| | | CAACCACCAGAGGACCCATACCGGAGAAAAACCTTACAAATGCCCCGAGTGTGGAAAGTCCTTC |
| | | AGCTCCCCCGCCGATCTGACAAGACATCAGAGAACCCACACCGGCGAAAAACCTTATAAATGTC |
| | | CCGAGTGTGGCAAAAGCTTCTCCGACAAGAAGGATCTGACAAGACACCAAAGGACCCACACCGG |
| | | CGAGAAACCTTATAAATGTCCCGAATGCGGAAAAAGCTTTAGCAGAAACGACGCTCTGACCGAA |
| | | CACCAGAGAACACATACCGGAGAGAAACCCTATAAATGTCCCGAGTGCGGAAAATCCTTCAGCA |
| | | CCACCGGCGCTCTGACAGAGCATCAGAGGACACACACCGGCAAAAAGACCTCCGCTAGCGGCAG |
| | | CGGCGGCGGCAGCGGCGGCAAGCGGCCCGCCGCCACCAAGAAGGCCGGCCAGGCCAAGAAGAAG |
| | | AAGGGCAGCTACCCCTACGACGTGCCCGACTACGCCTGAGCGGCCGCTTAATTAAGCTGCCTTC |
| | | TGCGGGGCTTGCCTTCTGGCCATGCCCTTCTTCTCTCCCTTGCACCTGTACCTCTTGGTCTTTG |
| | | AATAAAGCCTGAGTAGGAAGTCTAGAAAAAAAAAAAAAAAAAAAAAAAAAAAAAAAAAAAAAAA |
| | | AAAAAAAAAAAAAAAAAAAAAAAAAAAAAAAAAAAAAAAAA |
| MR- | G9A-CXCL1 | AGGAAATAAGAGAGAAAAGAAGAGTAAGAAGAAATATAAGAGCCACCATGGCCCCCAAGAAGAA |
| 29871 | ZF1 SP6 | GCGGAAGGTGGGCATCCACGGCGTGCCCGCCGCCTCGGGCGGGGGTGGCTCAGGAAATAGGGCT |
| (SEQ ID | | ATCAGAACCGAGAAGATCATCTGTAGGGACGTGGCTAGAGGCTACGAGAACGTGCCCATTCCTT |
| NO: 230) | | GCGTGAATGGCGTGGATGGCGAACCTTGCCCCGAGGACTACAAATACATCTCCGAGAACTGCGA |
| | | AACCAGCACAATGAACATCGACAGAAACATCACCCACCTCCAGCACTGCACATGTGTGGATGAC |
| | | TGCTCCTCCAGCAACTGTCTGTGCGGCCAGCTCTCCATCAGATGCTGGTACGACAAGGACGGCA |
| | | GACTGCTGCAAGAGTTCAACAAGATCGAACCCCCTCTCATCTTCGAGTGTAACCAAGCTTGCAG |
| | | CTGCTGGAGAAACTGCAAGAATAGAGTGGTCCAGAGCGGCATCAAGGTGAGACTGCAACTGTAC |
| | | AGAACCGCCAAGATGGGATGGGGAGTGAGGGCTCTGCAAACCATTCCCCAAGGCACCTTCATCT |
| | | GCGAATACGTGGGCGAACTGATCTCCGACGCCGAAGCTGACGTGAGAGAGGACGACAGCTATCT |
| | | CTTCGATCTGGACAATAAGGACGGCGAGGTGTACTGCATCGACGCTAGATATTACGGCAACATC |
| | | TCTAGATTCATCAACCACCTCTGCGATCCCAACATCATTCCCGTGAGGGTGTTCATGCTGCACC |
| | | AAGATCTGAGGTTCCCTAGAATCGCCTTCTTCAGCTCTAGAGACATCAGAACCGGCGAGGAGCT |
| | | GGGCTTCGATTACGGCGATAGATTCTGGGACATCAAGTCCAAGTACTTCACATGCCAGTGCGGC |
| | | AGCGAGAAGTGTAAGCACAGCGCTGAGGCCATTGCTCTGGAGCAGTCTAGACTGGCCAGACTGG |
| | | ATGGCAGCAGCGGATCCCTGGAGCCCGGCGAAAAGCCTTACAAATGTCCCGAATGCGGAAAGAG |
| | | CTTCAGCAGAGCCGACAATCTGACCGAACATCAGAGAACCCATACCGGAGAAAAACCTTACAAA |
| | | TGTCCCGAGTGCGGCAAAAGCTTCTCCCAAGCCGGACATCTGGCCAGCCACCAAAGGACACATA |
| | | CCGGCGAGAAACCCTACAAGTGCCCCGAGTGCGGCAAGTCCTTCTCTAGATCCGATGAGCTGGT |
| | | CAGACATCAGAGAACCCATACCGGCGAGAAGCCTTATAAGTGCCCCGAATGTGGCAAGTCCTTC |
| | | AGCCAGAGAGCTCATCTGGAGAGGCATCAAAGAACACACACCGGAGAGAAACCTTACAAGTGTC |
| | | CCGAGTGTGGAAAGAGCTTCTCCAGAAGGGACGAGCTGAACGTCCACCAAAGAACCCATACCGG |
| | | CGAAAAGCCCTATAAATGCCCCGAGTGTGGAAAATCCTTTTCTAGATCCGACCATCTGACAACC |
| | | CACCAGAGGACCCATACCGGAGAGAAGCCCTACAAATGCCCCGAGTGTGGAAAAAGCTTCTCTA |
| | | GAAACGATGCTCTGACAGAGCACCAAAGGACCCACACCGGCAAAAAGACCAGCGCTAGCGGCAG |
| | | CGGCGGCGGCAGCGGCGGCAAGCGGCCCGCCGCCACCAAGAAGGCCGGCCAGGCCAAGAAGAAG |
| | | AAGGGCAGCTACCCCTACGACGTGCCCGACTACGCCTGAGCGGCCGCTTAATTAAGCTGCCTTC |
| | | TGCGGGGCTTGCCTTCTGGCCATGCCCTTCTTCTCTCCCTTGCACCTGTACCTCTTGGTCTTTG |
| | | AATAAAGCCTGAGTAGGAAGTCTAGAAAAAAAAAAAAAAAAAAAAAAAAAAAAAAAAAAAAAAA |
| | | AAAAAAAAAAAAAAAAAAAAAAAAAAAAAAAAAAAAAAAAA |
| MR- | CXCL1 ZF2- | AGGAAATAAGAGAGAAAAGAAGAGTAAGAAGAAATATAAGAGCCACCATGGCCCCCAAGAAGAA |
| 29870 | DNMT3a-3L | GCGGAAGGTGGGCATCCACGGCGTGCCCGCCGCCGGCAGCAGCGGATCCCTGGAGCCCGGCGAA |
| (SEQ ID | SP6 | AAACCCTATAAGTGCCCCGAGTGCGGCAAGAGCTTTAGCGATCCCGGCCATCTGGTGAGGCATC |
| NO: 231) | | AGAGGACCCACACCGGCGAAAAGCCTTACAAATGCCCCGAGTGTGGAAAAAGCTTCAGCAGAAG |
| | | CGATCATCTGACCACCCATCAGAGGACACATACCGGCGAGAAGCCTTATAAATGCCCCGAATGT |
| | | GGAAAGAGCTTCTCCAGAAGCGACCATCTGACCAACCACCAGAGGACCCATACCGGAGAAAAAC |
| | | CTTACAAATGCCCCGAGTGTGGAAAGTCCTTCAGCTCCCCCGCCGATCTGACAAGACATCAGAG |
| | | AACCCACACCGGCGAAAAACCTTATAAATGTCCCGAGTGTGGCAAAAGCTTCTCCGACAAGAAG |
| | | GATCTGACAAGACACCAAAGGACCCACACCGGCGAGAAACCTTATAAATGTCCCGAATGCGGAA |
| | | AAAGCTTTAGCAGAAACGACGCTCTGACCGAACACCAGAGAACACATACCGGAGAGAAACCCTA |
| | | TAAATGTCCCGAGTGCGGAAAATCCTTCAGCACCACCGGCGCTCTGACAGAGCATCAGAGGACA |
| | | CACACCGGCAAAAAGACCTCCGCTAGCGGCAGCGGCGGCGGCAGCGGCGGCAACCACGACCAGG |
| | | AGTTCGACCCCCCCAAGGTGTACCCCCCCGTGCCCGCCGAGAAGCGGAAGCCCATCCGGGTGCT |
| | | GAGCCTGTTCGACGGCATCGCCACCGGCCTGCTGGTGCTGAAGGACCTGGGCATCCAGGTGGAC |
| | | CGGTACATCGCCAGCGAGGTGTGCGAGGACAGCATCACCGTGGGCATGGTGCGGCACCAGGGCA |
| | | AGATCATGTACGTGGGCGACGTGCGGAGCGTGACCCAGAAGCACATCCAGGAGTGGGGCCCCTT |
| | | CGACCTGGTGATCGGCGGCAGCCCCTGCAACGACCTGAGCATCGTGAACCCCGCCCGGAAGGGC |
| | | CTGTACGAGGGCACCGGCCGGCTGTTCTTCGAGTTCTACCGGCTGCTGCACGACGCCCGGCCCA |
| | | AGGAGGGCGACGACCGGCCCTTCTTCTGGCTGTTCGAGAACGTGGTGGCCATGGGCGTGAGCGA |
| | | CAAGCGGGACATCAGCCGGTTCCTGGAGAGCAACCCCGTGATGATCGACGCCAAGGAGGTGAGC |
| | | GCCGCCCACCGGGCCCGGTACTTCTGGGGCAACCTGCCCGGCATGAACCGGCCCCTGGCCAGCA |
| | | CCGTGAACGACAAGCTGGAGCTGCAGGAGTGCCTGGAGCACGGCCGGATCGCCAAGTTCAGCAA |
| | | GGTGCGGACCATCACCACCCGGAGCAACAGCATCAAGCAGGGCAAGGACCAGCACTTCCCCGTG |
| | | TTCATGAACGAGAAGGAGGACATCCTGTGGTGCACCGAGATGGAGCGGGTGTTCGGCTTCCCCG |
| | | TGCACTACACCGACGTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCGCCTGCGTGAGCAGC |
| | | GGCAACAGCAACGCCAACAGCCGGGGCCCCAGCTTCAGCAGCGGCCTGGTGCCCCTGAGCCTGC |
| | | GGGGCAGCCACATGAATCCTCTGGAGATGTTCGAGACAGTGCCCGTGTGGAGAAGGCAACCCGT |
| | | GAGGGTGCTGAGCCTCTTCGAGGACATTAAGAAGGAGCTGACCTCTCTGGGCTTTCTGGAATCC |
| | | GGCAGCGACCCCGGCCAGCTGAAACACGTGGTGGACGTGACCGACACAGTGAGGAAGGACGTGG |
| | | AAGAGTGGGGCCCCTTTGACCTCGTGTATGGAGCCACACCTCCTCTCGGCCACACATGCGATAG |
| | | GCCTCCCAGCTGGTATCTCTTCCAGTTCCACAGACTGCTCCAGTACGCCAGACCTAAGCCCGGC |
| | | AGCCCCAGACCCTTCTTCTGGATGTTCGTGGACAATCTGGTGCTGAACAAGGAGGATCTGGATG |
| | | TGGCCAGCAGATTTCTGGAGATGGAACCCGTGACAATCCCCGACGTGCATGGCGGCTCTCTGCA |
| | | GAACGCCGTGAGAGTGTGGTCCAACATCCCCGCCATTAGAAGCAGACACTGGGCTCTGGTGAGC |
| | | GAGGAGGAACTGTCTCTGCTGGCCCAGAATAAGCAGTCCTCCAAGCTGGCCGCCAAGTGGCCCA |
| | | CCAAGCTGGTGAAGAACTGCTTTCTGCCTCTGAGGGAGTATTTCAAGTATTTCAGCACCGAACT |
| | | GACCAGCAGCCTGAGCGGCGGCAAGCGGCCCGCCGCCACCAAGAAGGCCGGCCAGGCCAAGAAG |
| | | AAGAAGGGCAGCTACCCCTACGACGTGCCCGACTACGCCTGAGCGGCCGCTTAATTAAGCTGCC |
| | | TTCTGCGGGGCTTGCCTTCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAAAAAAAAAAAAAAAAAAA |
| | | AAAAAAAAAAAAAAAAAAAAAAAAAAAAAAAAAAAAAAAAAAAA |
| MR- | CXCL1 ZF6- | AGGAAATAAGAGAGAAAAGAAGAGTAAGAAGAAATATAAGAGCCACCATGGCCCCCAAGAAGAA |
| 29867 | HDAC8 SP6 | GCGGAAGGTGGGCATCCACGGCGTGCCCGCCGCCGGCAGCAGCGGATCCCTGGAGCCCGGCGAA |
| (SEQ ID | | AAGCCTTACAAGTGTCCCGAGTGCGGAAAGAGCTTCAGCAGAGCCGATAATCTGACCGAGCACC |
| NO: 232) | | AAAGGACCCACACCGGAGAGAAGCCTTATAAGTGTCCCGAATGCGGCAAAAGCTTTTCTAGAAG |
| | | CGATCATCTGACCAACCACCAGAGGACACACACCGGAGAAAAACCTTACAAATGCCCCGAGTGC |
| | | GGCAAAAGCTTCTCCCAGAGCAGCAATCTGGTGAGACACCAAAGGACCCACACCGGCGAAAAAC |
| | | CCTATAAATGCCCCGAATGTGGCAAGAGCTTTAGCACATCCGGCGAGCTGGTGAGGCATCAAAG |
| | | AACACATACCGGCGAGAAGCCCTACAAGTGCCCCGAGTGTGGAAAAAGCTTCAGCACCCACCTC |
| | | GATCTGATCAGACACCAGAGGACCCATACCGGAGAGAAACCCTACAAATGTCCCGAGTGCGGAA |
| | | AGTCCTTTAGCCAGCTGGCCCATCTGAGAGCTCATCAAAGGACACACACCGGCGAGAAGCCTTA |
| | | CAAGTGTCCCGAGTGCGGAAAATCCTTCTCCCAACTGGCCCATCTGAGGGCCCACCAGAGAACC |
| | | CACACCGGCAAAAAGACCTCCGCTAGCGGCAGCGGCGGCGGCAGCGGCGGCGAGGAGCCCGAGG |
| | | AGCCCGCCGATAGCGGACAATCTCTGGTGCCCGTCTACATCTACAGCCCCGAATATGTGAGCAT |
| | | GTGTGATTCCCTCGCCAAGATCCCTAAGAGAGCCAGCATGGTGCATTCTCTGATCGAGGCCTAC |
| | | GCTCTGCATAAGCAAATGAGGATCGTGAAGCCCAAGGTCGCCAGCATGGAAGAGATGGCCACCT |
| | | TTCACACCGATGCCTACCTCCAACATCTCCAGAAGGTGTCCCAAGAGGGCGACGACGACCACCC |
| | | CGACTCCATTGAGTACGGACTGGGCTATGATTGCCCCGCCACCGAGGGCATCTTTGACTATGCC |
| | | GCCGCTATCGGCGGAGCTACCATCACAGCCGCCCAGTGTCTGATTGATGGCATGTGCAAGGTCG |
| | | CCATCAACTGGTCCGGAGGCTGGCATCATGCCAAGAAGGATGAGGCCTCCGGCTTCTGTTATCT |
| | | GAATGACGCCGTGCTGGGCATTCTGAGACTGAGGAGGAAATTCGAGAGGATTCTGTACGTGGAT |
| | | CTGGATCTGCATCACGGAGATGGAGTCGAAGATGCCTTCAGCTTCACCAGCAAGGTGATGACAG |
| | | TCTCTCTGCACAAGTTCTCCCCCGGCTTCTTTCCCGGAACCGGCGACGTGTCCGACGTGGGACT |
| | | GGGCAAGGGAAGGTACTACAGCGTGAACGTGCCCATTCAAGACGGCATCCAAGACGAGAAGTAC |
| | | TACCAGATCTGCGAGTCCGTGCTCAAGGAGGTCTACCAAGCCTTCAATCCTAAGGCTGTCGTGC |
| | | TCCAACTGGGAGCTGATACCATTGCTGGCGATCCCATGTGCAGCTTCAATATGACACCCGTCGG |
| | | AATCGGCAAGTGCCTCAAGTACATCCTCCAGTGGCAGCTCGCCACCCTCATTCTCGGAGGAGGC |
| | | GGATACAATCTGGCTAATACCGCCAGATGCTGGACCTATCTGACCGGCGTGATTCTGGGCAAAA |
| | | CACTGAGCAGCGAAATCCCCGACCACGAGTTTTTCACCGCTTACGGCCCCGACTACGTGCTGGA |
| | | GATCACCCCCAGCTGCAGACCCGATAGAAACGAACCCCATAGAATCCAGCAAATTCTGAACTAT |
| | | ATCAAGGGCAACCTCAAGCACGTCGTGGGAGGTGGCGGATCGGGAAAGCGGCCCGCCGCCACCA |
| | | AGAAGGCCGGTCAGGCCAAGAAGAAGAAGGGCAGCTACCCCTACGACGTGCCCGACTACGCCTG |
| | | AGCGGCCGCTTAATTAAGCTGCCTTCTGCGGGGCTTGCCTTCTGGCCATGCCCTTCTTCTCTCC |
| | | CTTGCACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAAAAAA |
| | | AAA |
| MR- | EZH2- | AGGAAATAAGAGAGAAAAGAAGAGTAAGAAGAAATATAAGAGCCACCATGGCCCCCAAGAAGAA |
| 29850 | CXCL1 ZF4 | GCGGAAGGTGGGCGGCAGCGGCGGCAGCGGCCAGACCGGCAAGAAGAGCGAGAAGGGCCCCGTG |
| (SEQ ID | T7 | TGCTGGCGGAAGCGGGTGAAGAGCGAGTACATGCGGCTGCGGCAGCTGAAGCGGTTCCGGCGGG |
| NO: 233) | | CCGACGAGGTGAAGAGCATGTTCAGCAGCAACCGGCAGAAGATCCTGGAGCGGACCGAGATCCT |
| | | GAACCAGGAGTGGAAGCAGCGGCGAATCCAGCCCGTGCACATCCTGACCAGCGTGAGCAGCCTG |
| | | CGGGGCACCCGGGAGTGCAGCGTGACCAGCGACCTGGACTTCCCCACCCAGGTGATCCCCCTAA |
| | | AGACCCTGAACGCCGTGGCCAGCGTGCCCATCATGTACAGCTGGAGCCCCCTGCAGCAGAACTT |
| | | CATGGTGGAGGACGAGACCGTGCTGCACAACATCCCCTACATGGGCGACGAGGTGCTGGACCAG |
| | | GACGGCACCTTCATCGAGGAGCTGATCAAGAACTACGACGGCAAGGTGCACGGCGACCGGGAGT |
| | | GCGGCTTCATCAACGACGAGATCTTCGTGGAGCTGGTGAACGCCCTGGGCCAGTACAACGACGA |
| | | CGACGACGACGACGACGGCGACGACCCCGAGGAGCGGGAGGAGAAGCAGAAGGACCTGGAGGAC |
| | | CACCGGGACGACAAGGAGAGCCGGCCCCCCCGGAAGTTCCCCAGCGACAAGATCTTCGAGGCCA |
| | | TCAGCAGCATGTTCCCCGACAAGGGCACCGCCGAGGAGCTGAAGGAGAAGTACAAGGAGCTGAC |
| | | CGAGCAGCAGCTGCCCGGCGCCCTGCCCCCCGAGTGCACCCCCAACATCGACGGCCCCAACGCC |
| | | AAGAGCGTGCAGCGGGAGCAGAGCCTGCACAGCTTCCACACCCTGTTCTGCCGGCGGTGCTTCA |
| | | AGTACGACTGCTTCCTGCACCCCTTCCACGCCACCCCCAACACCTACAAGCGGAAGAACACCGA |
| | | GACCGCCCTGGACAACAAGCCCTGCGGCCCCCAGTGCTACCAGCACCTGGAGGGCGCCAAGGAG |
| | | TTCGCCGCCGCCCTGACCGCCGAGCGGATCAAGACCCCCCCCAAGCGGCCCGGCGGCCGGCGGG |
| | | GGGCCGGCTGCCCAACAACAGCAGCCGGCCCAGCACCCCCACCATCAACGTGCTGGAGAGCAAG |
| | | GACACCGACAGCGACCGGGAGGCCGGCACCGAGACCGGCGGCGAGAACAACGACAAGGAGGAGG |
| | | AGGAGAAGAAGGACGAGACCAGCAGCAGCAGCGAGGCCAACAGCCGGTGCCAGACCCCCATCAA |
| | | GATGAAGCCCAACATCGAGCCCCCCGAGAACGTGGAGTGGAGCGGCGCCGAGGCCAGCATGTTC |
| | | CGGGTGCTGATCGGCACCTACTACGACAACTTCTGCGCCATCGCCCGGCTGATCGGCACCAAGA |
| | | CCTGCCGGCAGGTGTACGAGTTCCGGGTGAAGGAGAGCAGCATCATCGCCCCCGCCCCCGCCGA |
| | | GGACGTGGACACCCCCCCCCGGAAGAAGAAGCGGAAGCACCGGCTGTGGGCCGCCCACTGCCGG |
| | | AAGATCCAGCTGAAGAAGGACGGCAGCAGCAACCACGTGTACAACTACCAGCCCTGCGACCACC |
| | | CCCGGCAGCCCTGCGACAGCAGCTGCCCCTGCGTGATCGCCCAGAACTTCTGCGAGAAGTTCTG |
| | | CCAGTGCAGCAGCGAGTGCCAGAACCGGTTCCCCGGCTGCCGGTGCAAGGCCCAGTGCAACACC |
| | | AAGCAGTGCCCCTGCTACCTGGCCGTGCGGGAGTGCGACCCCGACCTGTGCCTGACCTGCGGCG |
| | | CCGCCGACCACTGGGACAGCAAGAACGTGAGCTGCAAGAACTGCAGCATCCAGCGGGGCAGCAA |
| | | GAAGCACCTGCTGCTGGCCCCCAGCGACGTGGCCGGCTGGGGCATCTTCATCAAGGACCCCGTG |
| | | CAGAAGAACGAGTTCATCAGCGAGTACTGCGGCGAGATCATCAGCCAGGACGAGGCCGACCGGC |
| | | GGGGCAAGGTGTACGACAAGTACATGTGCAGCTTCCTGTTCAACCTGAACAACGACTTCGTGGT |
| | | GGACGCCACCCGGAAGGGCAACAAGATCCGGTTCGCCAACCACAGCGTGAACCCCAACTGCTAC |
| | | GCCAAGGTGATGATGGTGAACGGCGACCACCGGATCGGCATCTTCGCCAAGCGGGCCATCCAGA |
| | | CCGGCGAGGAGCTGTTCTTCGACTACCGGTACAGCCAGGCCGACGCCCTGAAGTACGTGGGCAT |
| | | CGAGCGGGAGATGGAGATCCCCGGCAGCAGCGGATCCCTGGAGCCCGGAGAAAAGCCTTACAAA |
| | | TGCCCCGAGTGCGGCAAGTCCTTCAGCCAGCTGGCTCATCTGAGAGCTCATCAAAGGACCCACA |
| | | CCGGCGAGAAGCCCTATAAGTGCCCCGAGTGCGGAAAATCCTTCTCCCAGAGCAGCAATCTCGT |
| | | CAGACACCAGAGGACCCACACCGGCGAGAAACCTTACAAGTGTCCCGAATGTGGAAAGTCCTTC |
| | | TCCCAAAAGAGCTCTCTGATCGCCCATCAGAGAACACATACCGGCGAAAAACCCTACAAGTGCC |
| | | CCGAGTGTGGCAAAAGCTTTTCCACCACCGGCAATCTGACCGTGCATCAAAGAACCCACACCGG |
| | | CGAAAAACCCTACAAATGCCCCGAGTGTGGCAAATCCTTCTCCGACCCCGGCCATCTGGTGAGG |
| | | CACCAGAGGACACACACCGGCGAGAAACCTTATAAATGTCCCGAATGCGGCAAGTCCTTTAGCA |
| | | CCAGCGGCTCTCTGGTGAGACATCAGAGGACACATACCGGCGAAAAGCCTTACAAGTGTCCCGA |
| | | GTGTGGCAAAAGCTTCAGCCAGAACAGCACACTGACAGAGCATCAGAGAACCCATACCGGCAAA |
| | | AAGACCAGCGCTAGCGGCAGCGGCGGCGGCAGCGGCGGCAAGCGGCCCGCCGCCACCAAGAAGG |
| | | CCGGCCAGGCCAAGAAGAAGAAGGGCAGCTACCCCTACGACGTGCCCGACTACGCCTGAGCGGC |
| | | CGCTTAATTAAGCTGCCTTCTGCGGGGCTTGCCTTCTGGCCATGCCCTTCTTCTCTCCCTTGCA |
| | | CCTGTACCTCTTGGTCTTTGAATAAAGCCTGAGTAGGAAGTCTAGAAAAAAAAAAAAAAAAAAA |
| | | AAAAAAAAAAAAAAAAAAAAAAAAAAAAAAAAAAAAAAAAAAAAAAAAAAAAAAAAAAAAA |
| MR- | EZH2- | AGGAAATAAGAGAGAAAAGAAGAGTAAGAAGAAATATAAGAGCCACCATGGCCCCCAAGAAGAA |
| 29849 | CXCL1 ZF3 | GCGGAAGGTGGGCGGCAGCGGCGGCAGCGGCCAGACCGGCAAGAAGAGCGAGAAGGGCCCCGTG |
| (SEQ ID | T7 | TGCTGGCGGAAGCGGGTGAAGAGCGAGTACATGCGGCTGCGGCAGCTGAAGCGGTTCCGGCGGG |
| NO: 234) | | CCGACGAGGTGAAGAGCATGTTCAGCAGCAACCGGCAGAAGATCCTGGAGCGGACCGAGATCCT |
| | | GAACCAGGAGTGGAAGCAGCGGCGAATCCAGCCCGTGCACATCCTGACCAGCGTGAGCAGCCTG |
| | | CGGGGCACCCGGGAGTGCAGCGTGACCAGCGACCTGGACTTCCCCACCCAGGTGATCCCCCTAA |
| | | AGACCCTGAACGCCGTGGCCAGCGTGCCCATCATGTACAGCTGGAGCCCCCTGCAGCAGAACTT |
| | | CATGGTGGAGGACGAGACCGTGCTGCACAACATCCCCTACATGGGCGACGAGGTGCTGGACCAG |
| | | GACGGCACCTTCATCGAGGAGCTGATCAAGAACTACGACGGCAAGGTGCACGGCGACCGGGAGT |
| | | GCGGCTTCATCAACGACGAGATCTTCGTGGAGCTGGTGAACGCCCTGGGCCAGTACAACGACGA |
| | | CGACGACGACGACGACGGCGACGACCCCGAGGAGCGGGAGGAGAAGCAGAAGGACCTGGAGGAC |
| | | CACCGGGACGACAAGGAGAGCCGGCCCCCCCGGAAGTTCCCCAGCGACAAGATCTTCGAGGCCA |
| | | TCAGCAGCATGTTCCCCGACAAGGGCACCGCCGAGGAGCTGAAGGAGAAGTACAAGGAGCTGAC |
| | | CGAGCAGCAGCTGCCCGGCGCCCTGCCCCCCGAGTGCACCCCCAACATCGACGGCCCCAACGCC |
| | | AAGAGCGTGCAGCGGGAGCAGAGCCTGCACAGCTTCCACACCCTGTTCTGCCGGCGGTGCTTCA |
| | | AGTACGACTGCTTCCTGCACCCCTTCCACGCCACCCCCAACACCTACAAGCGGAAGAACACCGA |
| | | GACCGCCCTGGACAACAAGCCCTGCGGCCCCCAGTGCTACCAGCACCTGGAGGGCGCCAAGGAG |
| | | TTCGCCGCCGCCCTGACCGCCGAGCGGATCAAGACCCCCCCCAAGCGGCCCGGCGGCCGGCGGC |
| | | GGGGCCGGCTGCCCAACAACAGCAGCCGGCCCAGCACCCCCACCATCAACGTGCTGGAGAGCAA |
| | | GGACACCGACAGCGACCGGGAGGCCGGCACCGAGACCGGCGGCGAGAACAACGACAAGGAGGAG |
| | | GAGGAGAAGAAGGACGAGACCAGCAGCAGCAGCGAGGCCAACAGCCGGTGCCAGACCCCCATCA |
| | | AGATGAAGCCCAACATCGAGCCCCCCGAGAACGTGGAGTGGAGCGGCGCCGAGGCCAGCATGTT |
| | | CCGGGTGCTGATCGGCACCTACTACGACAACTTCTGCGCCATCGCCCGGCTGATCGGCACCAAG |
| | | ACCTGCCGGCAGGTGTACGAGTTCCGGGTGAAGGAGAGCAGCATCATCGCCCCCGCCCCCGCCG |
| | | AGGACGTGGACACCCCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACCAGCCCTGCGACCAC |
| | | CCCCGGCAGCCCTGCGACAGCAGCTGCCCCTGCGTGATCGCCCAGAACTTCTGCGAGAAGTTCT |
| | | GCCAGTGCAGCAGCGAGTGCCAGAACCGGTTCCCCGGCTGCCGGTGCAAGGCCCAGTGCAACAC |
| | | CAAGCAGTGCCCCTGCTACCTGGCCGTGCGGGAGTGCGACCCCGACCTGTGCCTGACCTGCGGC |
| | | GCCGCCGACCACTGGGACAGCAAGAACGTGAGCTGCAAGAACTGCAGCATCCAGCGGGGCAGCA |
| | | AGAAGCACCTGCTGCTGGCCCCCAGCGACGTGGCCGGCTGGGGCATCTTCATCAAGGACCCCGT |
| | | GCAGAAGAACGAGTTCATCAGCGAGTACTGCGGCGAGATCATCAGCCAGGACGAGGCCGACCGG |
| | | CGGGGCAAGGTGTACGACAAGTACATGTGCAGCTTCCTGTTCAACCTGAACAACGACTTCGTGG |
| | | TGGACGCCACCCGGAAGGGCAACAAGATCCGGTTCGCCAACCACAGCGTGAACCCCAACTGCTA |
| | | CGCCAAGGTGATGATGGTGAACGGCGACCACCGGATCGGCATCTTCGCCAAGCGGGCCATCCAG |
| | | ACCGGCGAGGAGCTGTTCTTCGACTACCGGTACAGCCAGGCCGACGCCCTGAAGTACGTGGGCA |
| | | TCGAGCGGGAGATGGAGATCCCCGGCAGCAGCGGATCCCTGGAGCCCGGCGAAAAACCCTATAA |
| | | ATGCCCCGAGTGTGGCAAGAGCTTTTCCGACCCCGGACATCTCGTGAGGCATCAGAGAACACAT |
| | | ACCGGCGAGAAACCCTACAAGTGCCCCGAATGCGGCAAATCCTTCTCTAGAAAGGACAATCTGA |
| | | AAAACCATCAAAGAACCCATACCGGCGAGAAGCCCTATAAATGTCCCGAGTGTGGAAAGAGCTT |
| | | CAGCCACAAGAACGCTCTGCAGAACCATCAGAGGACCCATACCGGCGAAAAGCCTTATAAGTGC |
| | | CCCGAGTGCGGAAAATCCTTTTCTAGAAGGGACGAGCTGAATGTGCACCAAAGGACACATACCG |
| | | GAGAGAAACCCTACAAATGCCCCGAGTGCGGCAAGTCCTTCAGCACCTCCGGCAATCTGGTGAG |
| | | GCACCAAAGGACACACACCGGCGAAAAACCTTACAAGTGTCCCGAGTGCGGAAAAAGCTTTTCC |
| | | CAGAACAGCACACTGACCGAACACCAAAGGACCCACACCGGAGAGAAACCTTATAAATGTCCCG |
| | | AGTGTGGAAAGTCCTTTAGCCAGTCCGGCAATCTGACAGAGCATCAAAGAACCCACACCGGCAA |
| | | AAAGACCTCCGCTAGCGGCAGCGGCGGCGGCAGCGGCGGCAAGCGGCCCGCCGCCACCAAGAAG |
| | | GCCGGCCAGGCCAAGAAGAAGAAGGGCAGCTACCCCTACGACGTGCCCGACTACGCCTGAGCGG |
| | | CCGCTTAATTAAGCTGCCTTCTGCGGGGCTTGCCTTCTGGCCATGCCCTTCTTCTCTCCCTTGC |
| | | ACCTGTACCTCTTGGTCTTTGAATAAAGCCTGAGTAGGAAGTCTAGAAAAAAAAAAAAAAAAAA |
| | | AAAAAAAAAAAAAAAAAAAAAAAAAAAAAAAAAAAAAAAAAAAAAAAAAAAAAAAAAAAAAA |
| MR- | CXCL1 ZF3- | AGGAAATAAGAGAGAAAAGAAGAGTAAGAAGAAATATAAGAGCCACCATGGCCCCCAAGAAGAA |
| 29848 | HDAC8 SP6 | GCGGAAGGTGGGCATCCACGGCGTGCCCGCCGCCGGCAGCAGCGGATCCCTGGAGCCCGGCGAA |
| (SEQ ID | | AAACCCTATAAATGCCCCGAGTGTGGCAAGAGCTTTTCCGACCCCGGACACCTCGTGAGGCATC |
| NO: 235) | | AGAGAACACATACCGGCGAGAAACCCTACAAGTGCCCCGAATGCGGCAAATCCTTCTCTAGAAA |
| | | GGACAATCTGAAAAACCATCAAAGAACCCATACCGGCGAGAAGCCCTATAAATGTCCCGAGTGT |
| | | GGAAAGAGCTTCAGCCACAAGAACGCTCTGCAGAACCATCAGAGGACCCATACCGGCGAAAAGC |
| | | CTTATAAGTGCCCCGAGTGCGGAAAATCCTTTTCTAGAAGGGACGAGCTGAATGTGCACCAAAG |
| | | GACACATACCGGAGAGAAACCCTACAAATGCCCCGAGTGCGGCAAGTCCTTCAGCACCTCCGGC |
| | | AATCTGGTGAGGCACCAAAGGACACACACCGGCGAAAAACCTTACAAGTGTCCCGAGTGCGGAA |
| | | AAAGCTTTTCCCAGAACAGCACACTGACCGAACACCAAAGGACCCACACCGGAGAGAAACCTTA |
| | | TAAATGTCCCGAGTGTGGAAAGTCCTTTAGCCAGTCCGGCAATCTGACAGAGCATCAAAGAACC |
| | | CACACCGGCAAAAAGACCTCCGCTAGCGGCAGCGGCGGCGGCAGCGGCGGCGAGGAGCCCGAGG |
| | | AGCCCGCCGATAGCGGACAATCTCTGGTGCCCGTCTACATCTACAGCCCCGAATATGTGAGCAT |
| | | GTGTGATTCCCTCGCCAAGATCCCTAAGAGAGCCAGCATGGTGCATTCTCTGATCGAGGCCTAC |
| | | GCTCTGCATAAGCAAATGAGGATCGTGAAGCCCAAGGTCGCCAGCATGGAAGAGATGGCCACCT |
| | | TTCACACCGATGCCTACCTCCAACATCTCCAGAAGGTGTCCCAAGAGGGCGACGACGACCACCC |
| | | CGACTCCATTGAGTACGGACTGGGCTATGATTGCCCCGCCACCGAGGGCATCTTTGACTATGCC |
| | | GCCGCTATCGGCGGAGCTACCATCACAGCCGCCCAGTGTCTGATTGATGGCATGTGCAAGGTCG |
| | | CCATCAACTGGTCCGGAGGCTGGCATCATGCCAAGAAGGATGAGGCCTCCGGCTTCTGTTATCT |
| | | GAATGACGCCGTGCTGGGCATTCTGAGACTGAGGAGGAAATTCGAGAGGATTCTGTACGTGGAT |
| | | CTGGATCTGCATCACGGAGATGGAGTCGAAGATGCCTTCAGCTTCACCAGCAAGGTGATGACAG |
| | | TCTCTCTGCACAAGTTCTCCCCCGGCTTCTTTCCCGGAACCGGCGACGTGTCCGACGTGGGACT |
| | | GGGCAAGGGAAGGTACTACAGCGTGAACGTGCCCATTCAAGACGGCATCCAAGACGAGAAGTAC |
| | | TACCAGATCTGCGAGTCCGTGCTCAAGGAGGTCTACCAAGCCTTCAATCCTAAGGCTGTCGTGC |
| | | TCCAACTGGGAGCTGATACCATTGCTGGCGATCCCATGTGCAGCTTCAATATGACACCCGTCGG |
| | | AATCGGCAAGTGCCTCAAGTACATCCTCCAGTGGCAGCTCGCCACCCTCATTCTCGGAGGAGGC |
| | | GGATACAATCTGGCTAATACCGCCAGATGCTGGACCTATCTGACCGGCGTGATTCTGGGCAAAA |
| | | CACTGAGCAGCGAAATCCCCGACCACGAGTTTTTCACCGCTTACGGCCCCGACTACGTGCTGGA |
| | | GATCACCCCCAGCTGCAGACCCGATAGAAACGAACCCCATAGAATCCAGCAAATTCTGAACTAT |
| | | ATCAAGGGCAACCTCAAGCACGTCGTGGGAGGTGGCGGATCGGGAAAGCGGCCCGCCGCCACCA |
| | | AGAAGGCCGGTCAGGCCAAGAAGAAGAAGGGCAGCTACCCCTACGACGTGCCCGACTACGCCTG |
| | | AGCGGCCGCTTAATTAAGCTGCCTTCTGCGGGGCTTGCCTTCTGGCCATGCCCTTCTTCTCTCC |
| | | CTTGCACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAAAAAA |
| | | AAA |
| MR- | CXCL1 ZF6- | AGGAAATAAGAGAGAAAAGAAGAGTAAGAAGAAATATAAGAGCCACCATGGCCCCCAAGAAGAA |
| 29847 | DNMT3a-3L | GCGGAAGGTGGGCATCCACGGCGTGCCCGCCGCCGGCAGCAGCGGATCCCTGGAGCCCGGCGAA |
| (SEQ ID | SP6 | AAGCCTTACAAGTGTCCCGAGTGCGGAAAGAGCTTCAGCAGAGCCGATAATCTGACCGAGCACC |
| NO: 236) | | AAAGGACCCACACCGGAGAGAAGCCTTATAAGTGTCCCGAATGCGGCAAAAGCTTTTCTAGAAG |
| | | CGAAAAGCTTCTCCCAGAGCAGCAATCTGGTGAGACACCAAAGGACCCACACCGGCGAAAAACC |
| | | CTATTCATCTGACCAACCACCAGAGGACACACACCGGAGAAAAACCTTACAAATGCCCCGAGTG |
| | | CGGCAAAATGCCCCGAATGTGGCAAGAGCTTTAGCACATCCGGCGAGCTGGTGAGGCATCAAAG |
| | | AACACATACCGGCGAGAAGCCCTACAAGTGCCCCGAGTGTGGAAAAAGCTTCAGCACCCACCTC |
| | | GATCTGATCAGACACCAGAGGACCCATACCGGAGAGAAACCCTACAAATGTCCCGAGTGCGGAA |
| | | AGTCCTTTAGCCAGCTGGCCCATCTGAGAGCTCATCAAAGGACACACACCGGCGAGAAGCCTTA |
| | | CAAGTGTCCCGAGTGCGGAAAATCCTTCTCCCAACTGGCCCATCTGAGGGCCCACCAGAGAACC |
| | | CACACCGGCAAAAAGACCTCCGCTAGCGGCAGCGGCGGCGGCAGCGGCGGCAACCACGACCAGG |
| | | AGTTCGACCCCCCCAAGGTGTACCCCCCCGTGCCCGCCGAGAAGCGGAAGCCCATCCGGGTGCT |
| | | GAGCCTGTTCGACGGCATCGCCACCGGCCTGCTGGTGCTGAAGGACCTGGGCATCCAGGTGGAC |
| | | CGGTACATCGCCAGCGAGGTGTGCGAGGACAGCATCACCGTGGGCATGGTGCGGCACCAGGGCA |
| | | AGATCATGTACGTGGGCGACGTGCGGAGCGTGACCCAGAAGCACATCCAGGAGTGGGGCCCCTT |
| | | CGACCTGGTGATCGGCGGCAGCCCCTGCAACGACCTGAGCATCGTGAACCCCGCCCGGAAGGGC |
| | | CTGTACGAGGGCACCGGCCGGCTGTTCTTCGAGTTCTACCGGCTGCTGCACGACGCCCGGCCCA |
| | | AGGAGGGCGACGACCGGCCCTTCTTCTGGCTGTTCGAGAACGTGGTGGCCATGGGCGTGAGCGA |
| | | CAAGCGGGACATCAGCCGGTTCCTGGAGAGCAACCCCGTGATGATCGACGCCAAGGAGGTGAGC |
| | | GCCGCCCACCGGGCCCGGTACTTCTGGGGCAACCTGCCCGGCATGAACCGGCCCCTGGCCAGCA |
| | | CCGTGAACGACAAGCTGGAGCTGCAGGAGTGCCTGGAGCACGGCCGGATCGCCAAGTTCAGCAA |
| | | GGTGCGGACCATCACCACCCGGAGCAACAGCATCAAGCAGGGCAAGGACCAGCACTTCCCCGTG |
| | | TTCATGAACGAGAAGGAGGACATCCTGTGGTGCACCGAGATGGAGCGGGTGTTCGGCTTCCCCG |
| | | TGCACTACACCGACGTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCGCCTGCGTGAGCAGC |
| | | GGCAACAGCAACGCCAACAGCCGGGGCCCCAGCTTCAGCAGCGGCCTGGTGCCCCTGAGCCTGC |
| | | GGGGCAGCCACATGAATCCTCTGGAGATGTTCGAGACAGTGCCCGTGTGGAGAAGGCAACCCGT |
| | | GAGGGTGCTGAGCCTCTTCGAGGACATTAAGAAGGAGCTGACCTCTCTGGGCTTTCTGGAATCC |
| | | GGCAGCGACCCCGGCCAGCTGAAACACGTGGTGGACGTGACCGACACAGTGAGGAAGGACGTGG |
| | | AAGAGTGGGGCCCCTTTGACCTCGTGTATGGAGCCACACCTCCTCTCGGCCACACATGCGATAG |
| | | GCCTCCCAGCTGGTATCTCTTCCAGTTCCACAGACTGCTCCAGTACGCCAGACCTAAGCCCGGC |
| | | AGCCCCAGACCCTTCTTCTGGATGTTCGTGGACAATCTGGTGCTGAACAAGGAGGATCTGGATG |
| | | TGGCCAGCAGATTTCTGGAGATGGAACCCGTGACAATCCCCGACGTGCATGGCGGCTCTCTGCA |
| | | GAACGCCGTGAGAGTGTGGTCCAACATCCCCGCCATTAGAAGCAGACACTGGGCTCTGGTGAGC |
| | | GAGGAGGAACTGTCTCTGCTGGCCCAGAATAAGCAGTCCTCCAAGCTGGCCGCCAAGTGGCCCA |
| | | CCAAGCTGGTGAAGAACTGCTTTCTGCCTCTGAGGGAGTATTTCAAGTATTTCAGCACCGAACT |
| | | GACCAGCAGCCTGAGCGGCGGCAAGCGGCCCGCCGCCACCAAGAAGGCCGGCCAGGCCAAGAAG |
| | | AAGAAGGGCAGCTACCCCTACGACGTGCCCGACTACGCCTGAGCGGCCGCTTAATTAAGCTGCC |
| | | TTCTGCGGGGCTTGCCTTCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAAAAAAAAAAAAAAAAAAA |
| | | AAAAAAAAAAAAAAAAAAAAAAAAAAAAAAAAAAAAAAAAAAAA |
| MR- | CXCL1 ZF4- | AGGAAATAAGAGAGAAAAGAAGAGTAAGAAGAAATATAAGAGCCACCATGGCCCCCAAGAAGAA |
| 29846 | DNMT3a-3L | GCGGAAGGTGGGCATCCACGGCGTGCCCGCCGCCGGCAGCAGCGGATCCCTGGAGCCCGGAGAA |
| (SEQ ID | SP6 | AAGCCTTACAAATGCCCCGAGTGCGGCAAGTCCTTCAGCCAGCTGGCTCATCTGAGAGCTCATC |
| NO: 237) | | AAAGGACCCACACCGGCGAGAAGCCCTATAAGTGCCCCGAGTGCGGAAAATCCTTCTCCCAGAG |
| | | CAGCAATCTCGTCAGACACCAGAGGACCCACACCGGCGAGAAACCTTACAAGTGTCCCGAATGT |
| | | GGAAAGTCCTTCTCCCAAAAGAGCTCTCTGATCGCCCATCAGAGAACACATACCGGCGAAAAAC |
| | | CCTACAAGTGCCCCGAGTGTGGCAAAAGCTTTTCCACCACCGGCAATCTGACCGTGCATCAAAG |
| | | AACCCACACCGGCGAAAAACCCTACAAATGCCCCGAGTGTGGCAAATCCTTCTCCGACCCCGGC |
| | | CATCTGGTGAGGCACCAGAGGACACACACCGGCGAGAAACCTTATAAATGTCCCGAATGCGGCA |
| | | AGTCCTTTAGCACCAGCGGCTCTCTGGTGAGACATCAGAGGACACATACCGGCGAAAAGCCTTA |
| | | CAAGTGTCCCGAGTGTGGCAAAAGCTTCAGCCAGAACAGCACACTGACAGAGCATCAGAGAACC |
| | | CATACCGGCAAAAAGACCAGCGCTAGCGGCAGCGGCGGCGGCAGCGGCGGCAACCACGACCAGG |
| | | AGTTCGACCCCCCCAAGGTGTACCCCCCCGTGCCCGCCGAGAAGCGGAAGCCCATCCGGGTGCT |
| | | GAGCCTGTTCGACGGCATCGCCACCGGCCTGCTGGTGCTGAAGGACCTGGGCATCCAGGTGGAC |
| | | CGGTACATCGCCAGCGAGGTGTGCGAGGACAGCATCACCGTGGGCATGGTGCGGCACCAGGGCA |
| | | AGATCATGTACGTGGGCGACGTGCGGAGCGTGACCCAGAAGCACATCCAGGAGTGGGGCCCCTT |
| | | CGACCTGGTGATCGGCGGCAGCCCCTGCAACGACCTGAGCATCGTGAACCCCGCCCGGAAGGGC |
| | | CTGTACGAGGGCACCGGCCGGCTGTTCTTCGAGTTCTACCGGCTGCTGCACGACGCCCGGCCCA |
| | | AGGAGGGCGACGACCGGCCCTTCTTCTGGCTGTTCGAGAACGTGGTGGCCATGGGCGTGAGCGA |
| | | CAAGCGGGACATCAGCCGGTTCCTGGAGAGCAACCCCGTGATGATCGACGCCAAGGAGGTGAGC |
| | | GCCGCCCACCGGGCCCGGTACTTCTGGGGCAACCTGCCCGGCATGAACCGGCCCCTGGCCAGCA |
| | | CCGTGAACGACAAGCTGGAGCTGCAGGAGTGCCTGGAGCACGGCCGGATCGCCAAGTTCAGCAA |
| | | GGTGCGGACCATCACCACCCGGAGCAACAGCATCAAGCAGGGCAAGGACCAGCACTTCCCCGTG |
| | | TTCATGAACGAGAAGGAGGACATCCTGTGGTGCACCGAGATGGAGCGGGTGTTCGGCTTCCCCG |
| | | TGCACTACACCGACGTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCGCCTGCGTGAGCAGC |
| | | GGCAACAGCAACGCCAACAGCCGGGGCCCCAGCTTCAGCAGCGGCCTGGTGCCCCTGAGCCTGC |
| | | GGGGCAGCCACATGAATCCTCTGGAGATGTTCGAGACAGTGCCCGTGTGGAGAAGGCAACCCGT |
| | | GAGGGTGCTGAGCCTCTTCGAGGACATTAAGAAGGAGCTGACCTCTCTGGGCTTTCTGGAATCC |
| | | GGCAGCGACCCCGGCCAGCTGAAACACGTGGTGGACGTGACCGACACAGTGAGGAAGGACGTGG |
| | | AAGAGTGGGGCCCCTTTGACCTCGTGTATGGAGCCACACCTCCTCTCGGCCACACATGCGATAG |
| | | GCCTCCCAGCTGGTATCTCTTCCAGTTCCACAGACTGCTCCAGTACGCCAGACCTAAGCCCGGC |
| | | AGCCCCAGACCCTTCTTCTGGATGTTCGTGGACAATCTGGTGCTGAACAAGGAGGATCTGGATG |
| | | TGGCCAGCAGATTTCTGGAGATGGAACCCGTGACAATCCCCGACGTGCATGGCGGCTCTCTGCA |
| | | GAACGCCGTGAGAGTGTGGTCCAACATCCCCGCCATTAGAAGCAGACACTGGGCTCTGGTGAGC |
| | | GAGGAGGAACTGTCTCTGCTGGCCCAGAATAAGCAGTCCTCCAAGCTGGCCGCCAAGTGGCCCA |
| | | CCAAGCTGGTGAAGAACTGCTTTCTGCCTCTGAGGGAGTATTTCAAGTATTTCAGCACCGAACT |
| | | GACCAGCAGCCTGAGCGGCGGCAAGCGGCCCGCCGCCACCAAGAAGGCCGGCCAGGCCAAGAAG |
| | | AAGAAGGGCAGCTACCCCTACGACGTGCCCGACTACGCCTGAGCGGCCGCTTAATTAAGCTGCC |
| | | TTCTGCGGGGCTTGCCTTCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAAAAAAAAAAAAAAAAAAA |
| | | AAAAAAAAAAAAAAAAAAAAAAAAAAAAAAAAAAAAAAAAAAAA |
| MR- | CXCL1 ZF3- | AGGAAATAAGAGAGAAAAGAAGAGTAAGAAGAAATATAAGAGCCACCATGGCCCCCAAGAAGAA |
| 29845 | DNMT3a-3L | GCGGAAGGTGGGCATCCACGGCGTGCCCGCCGCCGGCAGCAGCGGATCCCTGGAGCCCGGCGAA |
| (SEQ ID | SP6 | AAACCCTATAAATGCCCCGAGTGTGGCAAGAGCTTTTCCGACCCCGGACACCTCGTGAGGCATC |
| NO: 238) | | AGAGAACACATACCGGCGAGAAACCCTACAAGTGCCCCGAATGCGGCAAATCCTTCTCTAGAAA |
| | | GGACAATCTGAAAAACCATCAAAGAACCCATACCGGCGAGAAGCCCTATAAATGTCCCGAGTGT |
| | | GGAAAGAGCTTCAGCCACAAGAACGCTCTGCAGAACCATCAGAGGACCCATACCGGCGAAAAGC |
| | | CTTATAAGTGCCCCGAGTGCGGAAAATCCTTTTCTAGAAGGGACGAGCTGAATGTGCACCAAAG |
| | | GACACATACCGGAGAGAAACCCTACAAATGCCCCGAGTGCGGCAAGTCCTTCAGCACCTCCGGC |
| | | AATCTGGTGAGGCACCAAAGGACACACACCGGCGAAAAACCTTACAAGTGTCCCGAGTGCGGAA |
| | | AAAGCTTTTCCCAGAACAGCACACTGACCGAACACCAAAGGACCCACACCGGAGAGAAACCTTA |
| | | TAAATGTCCCGAGTGTGGAAAGTCCTTTAGCCAGTCCGGCAATCTGACAGAGCATCAAAGAACC |
| | | CACACCGGCAAAAAGACCTCCGCTAGCGGCAGCGGCGGCGGCAGCGGCGGCAACCACGACCAGG |
| | | AGTTCGACCCCCCCAAGGTGTACCCCCCCGTGCCCGCCGAGAAGCGGAAGCCCATCCGGGTGCT |
| | | GAGCCTGTTCGACGGCATCGCCACCGGCCTGCTGGTGCTGAAGGACCTGGGCATCCAGGTGGAC |
| | | CGGTACATCGCCAGCGAGGTGTGCGAGGACAGCATCACCGTGGGCATGGTGCGGCACCAGGGCA |
| | | AGATCATGTACGTGGGCGACGTGCGGAGCGTGACCCAGAAGCACATCCAGGAGTGGGGCCCCTT |
| | | CGACCTGGTGATCGGCGGCAGCCCCTGCAACGACCTGAGCATCGTGAACCCCGCCCGGAAGGGC |
| | | CTGTACGAGGGCACCGGCCGGCTGTTCTTCGAGTTCTACCGGCTGCTGCACGACGCCCGGCCCA |
| | | AGGAGGGCGACGACCGGCCCTTCTTCTGGCTGTTCGAGAACGTGGTGGCCATGGGCGTGAGCGA |
| | | CAAGCGGGACATCAGCCGGTTCCTGGAGAGCAACCCCGTGATGATCGACGCCAAGGAGGTGAGC |
| | | GCCGCCCACCGGGCCCGGTACTTCTGGGGCAACCTGCCCGGCATGAACCGGCCCCTGGCCAGCA |
| | | CCGTGAACGACAAGCTGGAGCTGCAGGAGTGCCTGGAGCACGGCCGGATCGCCAAGTTCAGCAA |
| | | GGTGCGGACCATCACCACCCGGAGCAACAGCATCAAGCAGGGCAAGGACCAGCACTTCCCCGTG |
| | | TTCATGAACGAGAAGGAGGACATCCTGTGGTGCACCGAGATGGAGCGGGTGTTCGGCTTCCCCG |
| | | TGCACTACACCGACGTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCGCCTGCGTGAGCAGC |
| | | GGCAACAGCAACGCCAACAGCCGGGGCCCCAGCTTCAGCAGCGGCCTGGTGCCCCTGAGCCTGC |
| | | GGGGCAGCCACATGAATCCTCTGGAGATGTTCGAGACAGTGCCCGTGTGGAGAAGGCAACCCGT |
| | | GAGGGTGCTGAGCCTCTTCGAGGACATTAAGAAGGAGCTGACCTCTCTGGGCTTTCTGGAATCC |
| | | GGCAGCGACCCCGGCCAGCTGAAACACGTGGTGGACGTGACCGACACAGTGAGGAAGGACGTGG |
| | | AAGAGTGGGGCCCCTTTGACCTCGTGTATGGAGCCACACCTCCTCTCGGCCACACATGCGATAG |
| | | GCCTCCCAGCTGGTATCTCTTCCAGTTCCACAGACTGCTCCAGTACGCCAGACCTAAGCCCGGC |
| | | AGCCCCAGACCCTTCTTCTGGATGTTCGTGGACAATCTGGTGCTGAACAAGGAGGATCTGGATG |
| | | TGGCCAGCAGATTTCTGGAGATGGAACCCGTGACAATCCCCGACGTGCATGGCGGCTCTCTGCA |
| | | GAACGCCGTGAGAGTGTGGTCCAACATCCCCGCCATTAGAAGCAGACACTGGGCTCTGGTGAGC |
| | | GAGGAGGAACTGTCTCTGCTGGCCCAGAATAAGCAGTCCTCCAAGCTGGCCGCCAAGTGGCCCA |
| | | CCAAGCTGGTGAAGAACTGCTTTCTGCCTCTGAGGGAGTATTTCAAGTATTTCAGCACCGAACT |
| | | GACCAGCAGCCTGAGCGGCGGCAAGCGGCCCGCCGCCACCAAGAAGGCCGGCCAGGCCAAGAAG |
| | | AAGAAGGGCAGCTACCCCTACGACGTGCCCGACTACGCCTGAGCGGCCGCTTAATTAAGCTGCC |
| | | TTCTGCGGGGCTTGCCTTCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAAAAAAAAAAAAAAAAAAA |
| | | AAAAAAAAAAAAAAAAAAAAAAAAAAAAAAAAAAAAAAAAAAAA |
| MR- | CXCL1 ZF1- | AGGAAATAAGAGAGAAAAGAAGAGTAAGAAGAAATATAAGAGCCACCATGGCCCCCAAGAAGAA |
| 29787 | DNMT3a-3L | GCGGAAGGTGGGCATCCACGGCGTGCCCGCCGCCGGCAGCAGCGGATCCCTGGAGCCCGGCGAA |
| (SEQ ID | SP6 | AAGCCTTACAAATGTCCCGAATGCGGAAAGAGCTTCAGCAGAGCCGACAATCTGACCGAACATC |
| NO: 239) | | AGAGAACCCATACCGGAGAAAAACCTTACAAATGTCCCGAGTGCGGCAAAAGCTTCTCCCAAGC |
| | | CGGACATCTGGCCAGCCACCAAAGGACACATACCGGCGAGAAACCCTACAAGTGCCCCGAGTGC |
| | | GGCAAGTCCTTCTCTAGATCCGATGAGCTGGTCAGACATCAGAGAACCCATACCGGCGAGAAGC |
| | | CTTATAAGTGCCCCGAATGTGGCAAGTCCTTCAGCCAGAGAGCTCATCTGGAGAGGCATCAAAG |
| | | AACACACACCGGAGAGAAACCTTACAAGTGTCCCGAGTGTGGAAAGAGCTTCTCCAGAAGGGAC |
| | | GAGCTGAACGTCCACCAAAGAACCCATACCGGCGAAAAGCCCTATAAATGCCCCGAGTGTGGAA |
| | | AATCCTTTTCTAGATCCGACCATCTGACAACCCACCAGAGGACCCATACCGGAGAGAAGCCCTA |
| | | CAAATGCCCCGAGTGTGGAAAAAGCTTCTCTAGAAACGATGCTCTGACAGAGCACCAAAGGACC |
| | | CACACCGGCAAAAAGACCAGCGCTAGCGGCAGCGGCGGCGGCAGCGGCGGCAACCACGACCAGG |
| | | AGTTCGACCCCCCCAAGGTGTACCCCCCCGTGCCCGCCGAGAAGCGGAAGCCCATCCGGGTGCT |
| | | GAGCCTGTTCGACGGCATCGCCACCGGCCTGCTGGTGCTGAAGGACCTGGGCATCCAGGTGGAC |
| | | CGGTACATCGCCAGCGAGGTGTGCGAGGACAGCATCACCGTGGGCATGGTGCGGCACCAGGGCA |
| | | AGATCATGTACGTGGGCGACGTGCGGAGCGTGACCCAGAAGCACATCCAGGAGTGGGGCCCCTT |
| | | CGACCTGGTGATCGGCGGCAGCCCCTGCAACGACCTGAGCATCGTGAACCCCGCCCGGAAGGGC |
| | | CTGTACGAGGGCACCGGCCGGCTGTTCTTCGAGTTCTACCGGCTGCTGCACGACGCCCGGCCCA |
| | | AGGAGGGCGACGACCGGCCCTTCTTCTGGCTGTTCGAGAACGTGGTGGCCATGGGCGTGAGCGA |
| | | CAAGCGGGACATCAGCCGGTTCCTGGAGAGCAACCCCGTGATGATCGACGCCAAGGAGGTGAGC |
| | | GCCGCCCACCGGGCCCGGTACTTCTGGGGCAACCTGCCCGGCATGAACCGGCCCCTGGCCAGCA |
| | | CCGTGAACGACAAGCTGGAGCTGCAGGAGTGCCTGGAGCACGGCCGGATCGCCAAGTTCAGCAA |
| | | GGTGCGGACCATCACCACCCGGAGCAACAGCATCAAGCAGGGCAAGGACCAGCACTTCCCCGTG |
| | | TTCATGAACGAGAAGGAGGACATCCTGTGGTGCACCGAGATGGAGCGGGTGTTCGGCTTCCCCG |
| | | TGCACTACACCGACGTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCGCCTGCGTGAGCAGC |
| | | GGCAACAGCAACGCCAACAGCCGGGGCCCCAGCTTCAGCAGCGGCCTGGTGCCCCTGAGCCTGC |
| | | GGGGCAGCCACATGAATCCTCTGGAGATGTTCGAGACAGTGCCCGTGTGGAGAAGGCAACCCGT |
| | | GAGGGTGCTGAGCCTCTTCGAGGACATTAAGAAGGAGCTGACCTCTCTGGGCTTTCTGGAATCC |
| | | GGCAGCGACCCCGGCCAGCTGAAACACGTGGTGGACGTGACCGACACAGTGAGGAAGGACGTGG |
| | | AAGAGTGGGGCCCCTTTGACCTCGTGTATGGAGCCACACCTCCTCTCGGCCACACATGCGATAG |
| | | GCCTCCCAGCTGGTATCTCTTCCAGTTCCACAGACTGCTCCAGTACGCCAGACCTAAGCCCGGC |
| | | AGCCCCAGACCCTTCTTCTGGATGTTCGTGGACAATCTGGTGCTGAACAAGGAGGATCTGGATG |
| | | TGGCCAGCAGATTTCTGGAGATGGAACCCGTGACAATCCCCGACGTGCATGGCGGCTCTCTGCA |
| | | GAACGCCGTGAGAGTGTGGTCCAACATCCCCGCCATTAGAAGCAGACACTGGGCTCTGGTGAGC |
| | | GAGGAGGAACTGTCTCTGCTGGCCCAGAATAAGCAGTCCTCCAAGCTGGCCGCCAAGTGGCCCA |
| | | CCAAGCTGGTGAAGAACTGCTTTCTGCCTCTGAGGGAGTATTTCAAGTATTTCAGCACCGAACT |
| | | GACCAGCAGCCTGAGCGGCGGCAAGCGGCCCGCCGCCACCAAGAAGGCCGGCCAGGCCAAGAAG |
| | | AAGAAGGGCAGCTACCCCTACGACGTGCCCGACTACGCCTGAGCGGCCGCTTAATTAAGCTGCC |
| | | TTCTGCGGGGCTTGCCTTCTGGCCATGCCCTTCTTCTCTCCCTTGCACCTGTACCTCTTGGTCT |
| | | TTGAATAAAGCCTGAGTAGGAAGTCTAGAAAAAAAAAAAAAAAAAAAAAAAAAAAAAAAAAAAA |
| | | AAAAAAAAAAAAAAAAAAAAAAAAAAAAAAAAAAAAAAAAAAAA |
| MR- | CXCL1 ZF5- | AGGAAATAAGAGAGAAAAGAAGAGTAAGAAGAAATATAAGAGCCACCATGGCCCCCAAGAAGAA |
| 29783 | HDAC8 SP6 | GCGGAAGGTGGGCATCCACGGCGTGCCCGCCGCCGGCAGCAGCGGATCCCTGGAGCCCGGCGAA |
| (SEQ ID | | AAGCCTTACAAGTGCCCCGAGTGTGGCAAATCCTTTAGCACCACCGGAAATCTGACCGTCCACC |
| NO: 240) | | AGAGAACACATACCGGCGAGAAACCCTACAAGTGTCCCGAGTGCGGCAAATCCTTCAGCCAGCT |
| | | GGCCCATCTGAGAGCCCATCAAAGGACCCATACCGGCGAGAAACCTTACAAGTGTCCCGAATGT |
| | | GGAAAGTCCTTTAGCAGCCCCGCCGATCTGACAAGACATCAAAGAACCCACACCGGCGAGAAGC |
| | | CCTATAAATGTCCCGAGTGTGGAAAGTCCTTCAGCCAGAGCGGCAATCTGACCGAGCATCAAAG |
| | | AACCCATACCGGCGAAAAGCCCTATAAGTGCCCCGAATGCGGAAAAAGCTTCTCCACAAGCGGC |
| | | GAGCTGGTGAGACACCAAAGGACACATACCGGCGAAAAGCCTTATAAATGCCCCGAGTGCGGCA |
| | | AGAGCTTCTCTAGAAAGGACAATCTGAAGAACCACCAAAGAACACACACCGGCGAGAAGCCCTA |
| | | CAAATGCCCCGAGTGCGGCAAGAGCTTTAGCCAGTCCAGCAACCTCGTGAGACATCAGAGGACA |
| | | CATACCGGAAAAAAGACCAGCGCTAGCGGCAGCGGCGGCGGCAGCGGCGGCGAGGAGCCCGAGG |
| | | AGCCCGCCGATAGCGGACAATCTCTGGTGCCCGTCTACATCTACAGCCCCGAATATGTGAGCAT |
| | | GTGTGATTCCCTCGCCAAGATCCCTAAGAGAGCCAGCATGGTGCATTCTCTGATCGAGGCCTAC |
| | | GCTCTGCATAAGCAAATGAGGATCGTGAAGCCCAAGGTCGCCAGCATGGAAGAGATGGCCACCT |
| | | TTCACACCGATGCCTACCTCCAACATCTCCAGAAGGTGTCCCAAGAGGGCGACGACGACCACCC |
| | | CGACTCCATTGAGTACGGACTGGGCTATGATTGCCCCGCCACCGAGGGCATCTTTGACTATGCC |
| | | GCCGCTATCGGCGGAGCTACCATCACAGCCGCCCAGTGTCTGATTGATGGCATGTGCAAGGTCG |
| | | CCATCAACTGGTCCGGAGGCTGGCATCATGCCAAGAAGGATGAGGCCTCCGGCTTCTGTTATCT |
| | | GAATGACGCCGTGCTGGGCATTCTGAGACTGAGGAGGAAATTCGAGAGGATTCTGTACGTGGAT |
| | | CTGGATCTGCATCACGGAGATGGAGTCGAAGATGCCTTCAGCTTCACCAGCAAGGTGATGACAG |
| | | TCTCTCTGCACAAGTTCTCCCCCGGCTTCTTTCCCGGAACCGGCGACGTGTCCGACGTGGGACT |
| | | GGGCAAGGGAAGGTACTACAGCGTGAACGTGCCCATTCAAGACGGCATCCAAGACGAGAAGTAC |
| | | TACCAGATCTGCGAGTCCGTGCTCAAGGAGGTCTACCAAGCCTTCAATCCTAAGGCTGTCGTGC |
| | | TCCAACTGGGAGCTGATACCATTGCTGGCGATCCCATGTGCAGCTTCAATATGACACCCGTCGG |
| | | AATCGGCAAGTGCCTCAAGTACATCCTCCAGTGGCAGCTCGCCACCCTCATTCTCGGAGGAGGC |
| | | GGATACAATCTGGCTAATACCGCCAGATGCTGGACCTATCTGACCGGCGTGATTCTGGGCAAAA |
| | | CACTGAGCAGCGAAATCCCCGACCACGAGTTTTTCACCGCTTACGGCCCCGACTACGTGCTGGA |
| | | GATCACCCCCAGCTGCAGACCCGATAGAAACGAACCCCATAGAATCCAGCAAATTCTGAACTAT |
| | | ATCAAGGGCAACCTCAAGCACGTCGTGGGAGGTGGCGGATCGGGAAAGCGGCCCGCCGCCACCA |
| | | AGAAGGCCGGTCAGGCCAAGAAGAAGAAGGGCAGCTACCCCTACGACGTGCCCGACTACGCCTG |
| | | AGCGGCCGCTTAATTAAGCTGCCTTCTGCGGGGCTTGCCTTCTGGCCATGCCCTTCTTCTCTCC |
| | | CTTGCACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAAAAAA |
| | | AAA |
| MR- | CXCL1 ZF2- | AGGAAATAAGAGAGAAAAGAAGAGTAAGAAGAAATATAAGAGCCACCATGGCCCCCAAGAAGAA |
| 29782 | HDAC8 SP6 | GCGGAAGGTGGGCATCCACGGCGTGCCCGCCGCCGGCAGCAGCGGATCCCTGGAGCCCGGCGAA |
| (SEQ ID | | AAACCCTATAAGTGCCCCGAGTGCGGCAAGAGCTTTAGCGATCCCGGCCATCTGGTGAGGCATC |
| NO: 241) | | AGAGGACCCACACCGGCGAAAAGCCTTACAAATGCCCCGAGTGTGGAAAAAGCTTCAGCAGAAG |
| | | CGATCATCTGACCACCCATCAGAGGACACATACCGGCGAGAAGCCTTATAAATGCCCCGAATGT |
| | | GGAAAGAGCTTCTCCAGAAGCGACCATCTGACCAACCACCAGAGGACCCATACCGGAGAAAAAC |
| | | CTTACAAATGCCCCGAGTGTGGAAAGTCCTTCAGCTCCCCCGCCGATCTGACAAGACATCAGAG |
| | | AACCCACACCGGCGAAAAACCTTATAAATGTCCCGAGTGTGGCAAAAGCTTCTCCGACAAGAAG |
| | | GATCTGACAAGACACCAAAGGACCCACACCGGCGAGAAACCTTATAAATGTCCCGAATGCGGAA |
| | | AAAGCTTTAGCAGAAACGACGCTCTGACCGAACACCAGAGAACACATACCGGAGAGAAACCCTA |
| | | TAAATGTCCCGAGTGCGGAAAATCCTTCAGCACCACCGGCGCTCTGACAGAGCATCAGAGGACA |
| | | CACACCGGCAAAAAGACCTCCGCTAGCGGCAGCGGCGGCGGCAGCGGCGGCGAGGAGCCCGAGG |
| | | AGCCCGCCGATAGCGGACAATCTCTGGTGCCCGTCTACATCTACAGCCCCGAATATGTGAGCAT |
| | | GTGTGATTCCCTCGCCAAGATCCCTAAGAGAGCCAGCATGGTGCATTCTCTGATCGAGGCCTAC |
| | | GCTCTGCATAAGCAAATGAGGATCGTGAAGCCCAAGGTCGCCAGCATGGAAGAGATGGCCACCT |
| | | TTCACACCGATGCCTACCTCCAACATCTCCAGAAGGTGTCCCAAGAGGGCGACGACGACCACCC |
| | | CGACTCCATTGAGTACGGACTGGGCTATGATTGCCCCGCCACCGAGGGCATCTTTGACTATGCC |
| | | GCCGCTATCGGCGGAGCTACCATCACAGCCGCCCAGTGTCTGATTGATGGCATGTGCAAGGTCG |
| | | CCATCAACTGGTCCGGAGGCTGGCATCATGCCAAGAAGGATGAGGCCTCCGGCTTCTGTTATCT |
| | | GAATGACGCCGTGCTGGGCATTCTGAGACTGAGGAGGAAATTCGAGAGGATTCTGTACGTGGAT |
| | | CTGGATCTGCATCACGGAGATGGAGTCGAAGATGCCTTCAGCTTCACCAGCAAGGTGATGACAG |
| | | TCTCTCTGCACAAGTTCTCCCCCGGCTTCTTTCCCGGAACCGGCGACGTGTCCGACGTGGGACT |
| | | GGGCAAGGGAAGGTACTACAGCGTGAACGTGCCCATTCAAGACGGCATCCAAGACGAGAAGTAC |
| | | TACCAGATCTGCGAGTCCGTGCTCAAGGAGGTCTACCAAGCCTTCAATCCTAAGGCTGTCGTGC |
| | | TCCAACTGGGAGCTGATACCATTGCTGGCGATCCCATGTGCAGCTTCAATATGACACCCGTCGG |
| | | AATCGGCAAGTGCCTCAAGTACATCCTCCAGTGGCAGCTCGCCACCCTCATTCTCGGAGGAGGC |
| | | GGATACAATCTGGCTAATACCGCCAGATGCTGGACCTATCTGACCGGCGTGATTCTGGGCAAAA |
| | | CACTGAGCAGCGAAATCCCCGACCACGAGTTTTTCACCGCTTACGGCCCCGACTACGTGCTGGA |
| | | GATCACCCCCAGCTGCAGACCCGATAGAAACGAACCCCATAGAATCCAGCAAATTCTGAACTAT |
| | | ATCAAGGGCAACCTCAAGCACGTCGTGGGAGGTGGCGGATCGGGAAAGCGGCCCGCCGCCACCA |
| | | AGAAGGCCGGTCAGGCCAAGAAGAAGAAGGGCAGCTACCCCTACGACGTGCCCGACTACGCCTG |
| | | AGCGGCCGCTTAATTAAGCTGCCTTCTGCGGGGCTTGCCTTCTGGCCATGCCCTTCTTCTCTCC |
| | | CTTGCACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAAAAAA |
| | | AAA |
| MR- | CXCL1 ZF1- | AGGAAATAAGAGAGAAAAGAAGAGTAAGAAGAAATATAAGAGCCACCATGGCCCCCAAGAAGAA |
| 29780 | HDAC8 SP6 | GCGGAAGGTGGGCATCCACGGCGTGCCCGCCGCCGGCAGCAGCGGATCCCTGGAGCCCGGCGAA |
| (SEQ ID | | AAGCCTTACAAATGTCCCGAATGCGGAAAGAGCTTCAGCAGAGCCGACAATCTGACCGAACATC |
| NO: 242) | | AGAGAACCCATACCGGAGAAAAACCTTACAAATGTCCCGAGTGCGGCAAAAGCTTCTCCCAAGC |
| | | CGGACATCTGGCCAGCCACCAAAGGACACATACCGGCGAGAAACCCTACAAGTGCCCCGAGTGC |
| | | GGCAAGTCCTTCTCTAGATCCGATGAGCTGGTCAGACATCAGAGAACCCATACCGGCGAGAAGC |
| | | CTTATAAGTGCCCCGAATGTGGCAAGTCCTTCAGCCAGAGAGCTCATCTGGAGAGGCATCAAAG |
| | | AACACACACCGGAGAGAAACCTTACAAGTGTCCCGAGTGTGGAAAGAGCTTCTCCAGAAGGGAC |
| | | GAGCTGAACGTCCACCAAAGAACCCATACCGGCGAAAAGCCCTATAAATGCCCCGAGTGTGGAA |
| | | AATCCTTTTCTAGATCCGACCATCTGACAACCCACCAGAGGACCCATACCGGAGAGAAGCCCTA |
| | | CAAATGCCCCGAGTGTGGAAAAAGCTTCTCTAGAAACGATGCTCTGACAGAGCACCAAAGGACC |
| | | CACACCGGCAAAAAGACCAGCGCTAGCGGCAGCGGCGGCGGCAGCGGCGGCGAGGAGCCCGAGG |
| | | AGCCCGCCGATAGCGGACAATCTCTGGTGCCCGTCTACATCTACAGCCCCGAATATGTGAGCAT |
| | | GTGTGATTCCCTCGCCAAGATCCCTAAGAGAGCCAGCATGGTGCATTCTCTGATCGAGGCCTAC |
| | | GCTCTGCATAAGCAAATGAGGATCGTGAAGCCCAAGGTCGCCAGCATGGAAGAGATGGCCACCT |
| | | TTCACACCGATGCCTACCTCCAACATCTCCAGAAGGTGTCCCAAGAGGGCGACGACGACCACCC |
| | | CGACTCCATTGAGTACGGACTGGGCTATGATTGCCCCGCCACCGAGGGCATCTTTGACTATGCC |
| | | GCCGCTATCGGCGGAGCTACCATCACAGCCGCCCAGTGTCTGATTGATGGCATGTGCAAGGTCG |
| | | CCATCAACTGGTCCGGAGGCTGGCATCATGCCAAGAAGGATGAGGCCTCCGGCTTCTGTTATCT |
| | | GAATGACGCCGTGCTGGGCATTCTGAGACTGAGGAGGAAATTCGAGAGGATTCTGTACGTGGAT |
| | | CTGGATCTGCATCACGGAGATGGAGTCGAAGATGCCTTCAGCTTCACCAGCAAGGTGATGACAG |
| | | TCTCTCTGCACAAGTTCTCCCCCGGCTTCTTTCCCGGAACCGGCGACGTGTCCGACGTGGGACT |
| | | GGGCAAGGGAAGGTACTACAGCGTGAACGTGCCCATTCAAGACGGCATCCAAGACGAGAAGTAC |
| | | TACCAGATCTGCGAGTCCGTGCTCAAGGAGGTCTACCAAGCCTTCAATCCTAAGGCTGTCGTGC |
| | | TCCAACTGGGAGCTGATACCATTGCTGGCGATCCCATGTGCAGCTTCAATATGACACCCGTCGG |
| | | AATCGGCAAGTGCCTCAAGTACATCCTCCAGTGGCAGCTCGCCACCCTCATTCTCGGAGGAGGC |
| | | GGATACAATCTGGCTAATACCGCCAGATGCTGGACCTATCTGACCGGCGTGATTCTGGGCAAAA |
| | | CACTGAGCAGCGAAATCCCCGACCACGAGTTTTTCACCGCTTACGGCCCCGACTACGTGCTGGA |
| | | GATCACCCCCAGCTGCAGACCCGATAGAAACGAACCCCATAGAATCCAGCAAATTCTGAACTAT |
| | | ATCAAGGGCAACCTCAAGCACGTCGTGGGAGGTGGCGGATCGGGAAAGCGGCCCGCCGCCACCA |
| | | AGAAGGCCGGTCAGGCCAAGAAGAAGAAGGGCAGCTACCCCTACGACGTGCCCGACTACGCCTG |
| | | AGCGGCCGCTTAATTAAGCTGCCTTCTGCGGGGCTTGCCTTCTGGCCATGCCCTTCTTCTCTCC |
| | | CTTGCACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAAAAAA |
| | | AAA |

In some embodiments, a nucleic acid for use in a method or composition described herein (e.g., a nucleic acid encoding a site-specific disrupting agent) comprises a nucleic acid sequence of any one of SEQ ID NOs: 69, 71, 85, 201, 202, 204, 205, 207, 209, 211, 213, 215, 217, or 219-242, a complementary or reverse complementary sequence of any thereof, or comprises a sequence with at least 80, 85, 90, 91, 92, 93, 94, 95, 96, 97, 98, or 99% identity to any thereof. In some embodiments, a site-specific disrupting agent for use in a method or composition described herein comprises an amino acid sequence of any one of SEQ ID NOs: 70, 72-82, 84, 86, 203, 206, 208, 210, 212, 214, 216, or 218, or comprises a sequence with at least 80, 85, 90, 91, 92, 93, 94, 95, 96, 97, 98, or 99% identity to any thereof. In some embodiments, a site-specific disrupting agent for use in a method or composition described herein comprises an amino acid sequence encoded by any one of SEQ ID NOs: 69, 71, 85, 201, 202, 204, 205, 207, 209, 211, 213, 215, 217, or 219-242, or an amino acid sequence with at least 80, 85, 90, 91, 92, 93, 94, 95, 96, 97, 98, or 99% identity to any thereof.

### Functional Characteristics

An expression repressor or a site-specific disrupting agent or a system of the present disclosure can be used to modulate, e.g., decrease, expression of a target plurality of genes in a cell. In some embodiments, modulating expression comprises decreasing the level of RNA, e.g., mRNA, encoded by each of the target plurality of genes. In some embodiments, modulating expression comprises decreasing the level of protein encoded by each of the target plurality of genes. In some embodiments, modulating expression comprises both decreasing the level of mRNA and protein encoded by each of the target plurality of genes. In some embodiments, the expression of a gene of the target plurality of genes in a cell contacted by or comprising the expression repressor or site-specific disrupting agent is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× lower than the level of expression of the gene in a cell not contacted by or comprising the expression repressor or system or the site-specific disrupting agent or system. In some embodiments, the expression of each gene of the target plurality of genes in a cell contacted by or comprising the expression repressor or system or the site-specific disrupting agent or system is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×. 6×, 7×, 8×, 9×, 10×, 20×, 30×, 40×, 50×, 60×, 70×, 80×, 90×, or 100× lower than the level of expression of the gene in a cell not contacted by or comprising the expression repressor or system or the site-specific disrupting agent or system. Expression of a gene may be assayed by methods known to those of skill in the art, including RT-PCR, ELISA, Western blot, and the methods of Examples 2 or 4-19.

Without wishing to be bound by theory, an expression repressor or a system of the present disclosure can be used to decrease binding of a factor to an enhancer sequence. In some embodiments, contacting a cell or administering an expression repressor or a system results in a decrease in binding of a factor to an enhancer sequence. In some embodiments, contacting a cell or administering an expression repressor or a system results in a complete loss of binding or a decrease of at least 50, 60, 70, 80, 90, 95, or 99%, e.g., as measured by ChIP and/or quantitative PCR, relative to the binding of the factor to the enhancer prior to treatment with the expression repressor or a system or in the absence of the expression repressor or a system.

An expression repressor or a system of the present disclosure can be used to modulate, e.g., decrease, expression of a target plurality of genes in a cell for a time period. In some embodiments, the expression of a gene of the target plurality of genes in a cell contacted by or comprising the expression repressor or a system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or weeks, or at least 1, 2, 3, 4, 5, 6, 7, 8, 9, 10, 11, or 12 months, or at least 1, 2, 3, 4, or 5 years (e.g., indefinitely). In some embodiments, the expression of each gene of the target plurality of genes in a cell contacted by or comprising the expression repressor or a system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, the expression of a gene of the target plurality of genes in a cell contacted by or comprising the expression repressor or a system is appreciably decreased for no more than 10, 9, 8, 7, 6, 5, 4, 3, 2, or 1 years. Optionally, the expression of each gene of the target plurality of genes in a cell contacted by or comprising the expression repressor or a system is appreciably decreased for no more than 10, 9, 8, 7, 6, 5, 4, 3, 2, or 1 years.

An expression repressor or a system may comprise a plurality of effector moieties, where each effector moiety has a different functionality from each other effector moiety. For example, an expression repressor or a system may comprise a first effector moiety comprising histone deacetylase functionality and a second effector moiety comprising DNA methyltransferase functionality. In some embodiments, an expression repressor comprises a combination of effector moieties whose functionalities are complementary to one another with regard to modulating, e.g., decreasing, expression of a target plurality of genes, e.g., where the functionalities together decrease expression and, optionally, do not decrease or negligibly decrease expression when present individually.

In some embodiments, an expression repressor or a system comprises a combination of effector moieties whose functionalities synergize with one another with regard to modulating, e.g., decreasing, expression of a target plurality of genes. Without wishing to be bound by theory, it is thought that epigenetic modifications to a genomic locus are cumulative, in that multiple repressive epigenetic markers (e.g., multiple different types of epigenetic markers and/or more extensive marking of a given type) together reduce expression more effectively than individual modifications alone (e.g., producing a greater decrease in expression and/or a longer-lasting decrease in expression). In some embodiments, an expression repressor or a system comprises a plurality of effector moieties that synergize with each other, e.g., each effector moiety decreases expression of a target gene. In some embodiments, an expression repressor comprising a plurality of different effector moieties which synergize with one another is more effective at modulating, e.g., decreasing, expression of a target plurality of genes than an expression repressor comprising only one of the plurality of different effector moieties or a non-synergistic combination of effector moieties. In some embodiments, such an expression repressor agent is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× as effective at modulating, e.g., decreasing, expression of a target plurality of genes than an expression repressor or a system comprising only one of the plurality of different effector moieties or a non-synergistic combination of effector moieties.

A site-specific disrupting agent or a system of the present disclosure can be used to decrease binding of a nucleating polypeptide, e.g., CTCF, to an anchor sequence. In some embodiments, contacting a cell or administering a site-specific disrupting agent or a system results in a decrease in binding of a nucleating polypeptide, e.g., CTCF, to an anchor sequence (e.g., an anchor sequence of an ASMC comprising a target plurality of genes). In some embodiments, contacting a cell or administering a site-specific disrupting agent or a system results in a complete loss of binding or a decrease of at least 50, 60, 70, 80, 90, 95, or 99%, e.g., as measured by ChIP and/or quantitative PCR, relative to the binding of the nucleating polypeptide (e.g., CTCF) to the anchor sequence prior to treatment with the site-specific disrupting agent or a system or in the absence of the site-specific disrupting agent or a system.

A site-specific disrupting agent or a system of the present disclosure can be used to disrupt a genomic complex (e.g., ASMC) comprising a target plurality of cells. In some embodiments, contacting a cell or administering a site-specific disrupting agent or a system results in a decrease in the level of a genomic complex (e.g., ASMC) comprising the target plurality of genes relative to the level of the complex prior to treatment with the site-specific disrupting agent or a system or in the absence of the site-specific disrupting agent or a system. In some embodiments, contacting a cell or administering a site-specific disrupting agent or a system results in a complete loss of the genomic complex, e.g., ASMC, or a decrease of at least 25, 30, 35, 40, 45, 50, 60, 70, 80, 90, 95, or 99%, e.g., as measured by ChIA-PET, ELISA (e.g., to assess gene expression changes), CUT&RUN, ATAC-SEQ, ChIP and/or quantitative PCR, relative to the level of the complex prior to treatment with the site-specific disrupting agent or a system or in the absence of the site-specific disrupting agent or a system.

A site-specific disrupting agent or a system of the present disclosure can be used to modulate, e.g., decrease, expression of a target plurality of genes in a cell for a time period. In some embodiments, the expression of a gene of the target plurality of genes in a cell contacted by or comprising the site-specific disrupting agent or a system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or years (e.g., indefinitely). In some embodiments, the expression of each gene of the target plurality of genes in a cell contacted by or comprising the site-specific disrupting agent or a system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, the expression of a gene of the target plurality of genes in a cell contacted by or comprising the site-specific disrupting agent or a system is appreciably decreased for no more than 10, 9, 8, 7, 6, 5, 4, 3, 2, or 1 years. Optionally, the expression of each gene of the target plurality of genes in a cell contacted by or comprising the site-specific disrupting agent or a system is appreciably decreased for no more than 10, 9, 8, 7, 6, 5, 4, 3, 2, or 1 years.

A site-specific disrupting agent or a system may comprise a plurality of effector moieties, where each effector moiety has a different functionality from each other effector moiety. For example, a site-specific disrupting agent or a system may comprise a first effector moiety comprising histone deacetylase functionality and a second effector moiety comprising DNA methyltransferase functionality. In some embodiments, a site-specific disrupting agent comprises a combination of effector moieties whose functionalities are complementary to one another with regard to modulating, e.g., decreasing, expression of a target plurality of genes, e.g., where the functionalities together decrease expression and, optionally, do not decrease or negligibly decrease expression when present individually.

In some embodiments, a site-specific disrupting agent or a system comprises a combination of effector moieties whose functionalities synergize with one another with regard to modulating, e.g., decreasing, expression of a target plurality of genes. Without wishing to be bound by theory, it is thought that epigenetic modifications to a genomic locus are cumulative, in that multiple repressive epigenetic markers (e.g., multiple different types of epigenetic markers and/or more extensive marking of a given type) together reduce expression more effectively than individual modifications alone (e.g., producing a greater decrease in expression and/or a longer-lasting decrease in expression). In some embodiments, a site-specific disrupting agent or a system comprises a plurality of effector moieties that synergize with each other, e.g., each effector moiety decreases expression of a target gene. In some embodiments, a site-specific disrupting agent comprising a plurality of different effector moieties which synergize with one another is more effective at modulating, e.g., decreasing, expression of a target plurality of genes than a site-specific disrupting agent comprising only one of the plurality of different effector moieties or a non-synergistic combination of effector moieties. In some embodiments, such a site-specific disrupting agent is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× as effective at modulating, e.g., decreasing, expression of a target plurality of genes than a site-specific disrupting agent or a system comprising only one of the plurality of different effector moieties or a non-synergistic combination of effector moieties.

### Target Sites

An expression repressor or a system disclosed herein is useful for modulating, e.g., decreasing, expression of a target plurality of genes in cell, e.g., in a subject or patient.

In combination with expression repressors or systems disclosed herein, a site-specific disrupting agent or a system disclosed herein is useful for modulating, e.g., decreasing, expression of a target plurality of genes in cell, e.g., in a subject or patient.

A target plurality of genes may include any gene known to those of skill in the art. A target plurality of genes comprises at least two genes. In some embodiments, a targeted plurality of genes comprises at least 2, 3, 4, 5, 6, 7, 8, 9, 10, 11, 12, 13, 14, 15, 16, 17, 18, 19, or 20 genes (and optionally, no more than 2, 3, 4, 5, 6, 7, 8, 9, 10, 11, 12, 13, 14, 15, 16, 17, 18, 19, 20, or 30 genes), e.g., a first gene and a second gene, and optionally a third gene, a fourth gene, a fifth gene, a sixth gene, a seventh gene, an eighth gene, a ninth gene, a tenth gene, an eleventh gene, a twelfth gene, a thirteenth gene, a fourteenth gene, a fifteenth gene, a sixteenth gene, a seventeenth gene, an eighteenth gene, a nineteenth gene, and/or a twentieth gene. In some embodiments, a targeted plurality of genes comprises 2-20, 2-18, 2-16, 2-14, 2-12, 2-10, 2-9, 2-8, 2-7, 2-6, 2-5, 2-4, 2-3, 3-20, 3-18, 3-16, 3-14, 3-12, 3-10, 3-9, 3-8, 3-7, 3-6, 3-5, 3-4, 4-20, 4-18, 4-16, 4-14, 4-12, 4-10, 4-9, 4-8, 4-7, 4-6, 4-5, 5-20, 5-18, 5-16, 5-14, 5-12, 5-10, 5-9, 5-8, 5-7, 5-6, 6-20, 6-18, 6-16, 6-14, 6-12, 6-10, 6-9, 6-8, 6-7, 7-20, 7-18, 7-16, 7-14, 7-12, 7-10, 7-9, 7-8, 8-20, 8-18, 8-16, 8-14, 8-12, 8-10, 8-9, 9-20, 9-18, 9-16, 9-14, 9-12, 9-10, 10-20, 10-18, 10-16, 10-14, 10-12, 12-20, 12-18, 12-16, 12-14, 14-20, 14-18, 14-16, 16-20, 16-18, or 18-20 genes.

In some embodiments, two or more (e.g., all) genes of a target plurality of genes are associated with a disease or condition in a subject, e.g., a mammal, e.g., a human, bovine, horse, sheep, chicken, rat, mouse, cat, or dog. In some embodiments, the disease or condition is an inflammatory disease, e.g., an immune mediated inflammatory disease. In some embodiments, the disease or condition is one or more of rheumatoid arthritis, inflammatory, arthritis, gout, asthma, neutrophilic asthma, neutrophilic dermatosis, paw edema, acute respiratory disease syndrome (ARDS), COVID-19, psoriasis, inflammatory bowel disease, infection (e.g., by a pathogen, e.g., a bacteria, a virus, or a fungus), external injury (e.g., scrapes or foreign objects), effects of radiation or chemical injury, osteoarthritis, osteoarthritic joint pain, joint pain, inflammatory pain, acute pain, chronic pain, cystitis, bronchitis, dermatitis, cardiovascular disease, neurodegenerative disease, liver disease, lung disease, kidney disease, pain, swelling, stiffness, tenderness, redness, warmth, or elevated biomarkers related to disease states (e.g., cytokines, immune receptors, or inflammatory markers). In some embodiments, the inflammatory disorder is associated with an infection, e.g., by a virus, e.g., Sars-Cov-2 virus. In some embodiments, the inflammatory disorder is an autoimmune disorder. In some embodiments, the inflammatory disorder is associated with hypoxia. In some embodiments, the inflammatory disorder is associated with ARDS, hypoxia, and/or sepsis. In some embodiments, the infection is a super infection, e.g., caused by more than one pathogen, e.g., a first virus or a bacterium, or a fungus, and a second virus, or a bacterium, or a fungus. In some embodiments, the inflammatory disorder may change lung cell composition, e.g., decreased AT2 cells and/or increased dendritic cell, macrophages, neutrophils, NK cells, fibroblasts, leukocytes, lymphatic endothelial cells and/or vascular endothelial cells. In some embodiments, the disorder is associated with one or more comorbidities, e.g., respiratory infections, obesity, gastroesophageal reflux disease, skin lesions, and/or obstructive sleep apnea.

In some embodiments, two or more (e.g., all) genes of a target plurality of genes are aberrantly expressed, e.g., over-expressed, in a cell, e.g., in a subject, e.g., a human subject.

In some embodiments, two or more (e.g., all) genes of a target plurality of genes have related functionalities. Without wishing to be bound by theory, it is thought that genes with related functionalities are frequently positioned in close proximity to one another in the genome and are also frequently found within (wholly or in part) common genomic complexes, e.g., ASMCs. Modulating, e.g., decreasing, expression of a target plurality of genes where two or more (e.g., all) of the genes of the plurality have related functionalities may be accomplished efficiently and effectively by targeting a genomic complex, e.g., ASMC, comprising said interrelated genes.

In some embodiments, one, two, three, or more (e.g., all) genes of a target plurality of genes are cytokines, e.g., chemokines, an interleukin, a transcription factor (e.g., an interferon regulatory transcription factor), intercellular adhesion molecule (ICAM), or an interferon receptor. In some embodiments, two or more (e.g., all) genes of a target plurality of genes are cytokines, an interleukin, a transcription factor (e.g., an interferon regulatory transcription factor), intercellular adhesion molecule (ICAM), or an interferon receptor. In some embodiments, the target plurality of genes are mammalian gene, e.g., mouse genes, human genes.

In some embodiments, two or more (e.g., all) genes of a target plurality of genes have pro-inflammatory functionality. In some embodiments, two or more (e.g., all) genes of a target plurality of genes may act as a chemoattractant for immune cells, e.g., neutrophils. For example, genes having pro-inflammatory functionality (also referred to herein as pro-inflammatory genes) include CXCL1, CXCL2, CXCL3, CXCL4, CXCL5, CXCL6, CXCL7, IL8, CXCL15, CCL2, CCL7, CCL9, IL1A, IL1B, CSF2, IRF1, ICAM1, ICAM4, ICAM5, IFNAR2, IL10RB, or IFNGR2. In some embodiments, a target plurality of genes comprises two or more of CXCL1, CXCL2, CXCL3, CXCL4, CXCL5, CXCL6, CXCL7, IL8, CXCL15, CCL2, CCL7, CCL9, IL1A, IL1B, CSF2, IRF1, ICAM1, ICAM4, ICAM5, IFNAR2, IL10RB, or IFNGR2. In some embodiments, the plurality of genes comprises one or more genes more human CXCL family. In some embodiments, a target plurality of genes comprises CXCL1 (e.g., nucleic acid sequence encoding an RNA according to NM_002089 or a nucleic acid encoding a polypeptide according to P09341, or a mutant thereof), CXCL2 (e.g., nucleic acid sequence encoding an RNA according to NM_001511 or a nucleic acid encoding a polypeptide according to P19875, or a mutant thereof), CXCL3 (e.g., nucleic acid sequence encoding an RNA according to NM_002090 or a nucleic acid encoding a polypeptide according to P19876, or a mutant thereof), CXCL4 (e.g., nucleic acid sequence encoding an RNA according to NM_002619 or NM_001363352, or a nucleic acid encoding a polypeptide according to P02776, or a mutant thereof), CXCL5 (e.g., nucleic acid sequence encoding an RNA according to NM_002994 or a nucleic acid encoding a polypeptide according to P42830, or a mutant thereof), CXCL6 (e.g., nucleic acid sequence encoding an RNA according to NM_002993 or a nucleic acid encoding a polypeptide according to P80162, or a mutant thereof), CXCL7 (e.g., nucleic acid sequence encoding an RNA according to NM_002704 or a nucleic acid encoding a polypeptide according to P02775, or a mutant thereof), and IL8 (also known as CXCL8, e.g., nucleic acid sequence encoding an RNA according to NM_000584 or NM_001354840, or a nucleic acid encoding a polypeptide according to P10145, or a mutant thereof). In some embodiments, the plurality of genes comprises one or more genes more mouse CXCL family. In some embodiments, a target plurality of genes comprises CXCL1 (e.g., nucleic acid sequence encoding an RNA according to NM_008176.3 or a nucleic acid encoding a polypeptide according to P12850, or a mutant thereof), CXCL2 (e.g., nucleic acid sequence encoding an RNA according to NM_009140.2 or a nucleic acid encoding a polypeptide according to P10889, or a mutant thereof), CXCL3 (e.g., nucleic acid sequence encoding an RNA according to NM_203320.3 or a nucleic acid encoding a polypeptide according to Q6W5C0, or a mutant thereof), CXCL4 (e.g., nucleic acid sequence encoding an RNA according to NM_019932 or a nucleic acid encoding a polypeptide according to Q9Z126, or a mutant thereof), CXCL5 (e.g., nucleic acid sequence encoding an RNA according to NM_009141.3 or a nucleic acid encoding a polypeptide according to P50228, or a mutant thereof), CXCL7 (e.g., nucleic acid sequence encoding an RNA according to NM 023785.3 or a nucleic acid encoding a polypeptide according to Q9EQI5, or a mutant thereof), and CXCL15 (e.g., nucleic acid sequence encoding an RNA according to NM_011339 or a nucleic acid encoding a polypeptide according to Q9WVL7, or a mutant thereof). In some embodiments, a target plurality of genes comprises CXCL1, CXCL2, CXCL3, and IL8. In some embodiments, a target plurality of genes is CXCL1, CXCL2, CXCL3, and IL8. In some embodiments, a target plurality of genes comprises CCL2, CCL7, CCL9, IL1A, and IL1B. In some embodiments, a target plurality of genes comprises CSF2, IRF1, ICAM1, ICAM4, and ICAM5. In some embodiments, a target plurality of genes comprises IFNAR2, IL10RB, and IFNGR2.

In some embodiments, inhibition expression of two or more (e.g., all) genes of a target plurality of genes may modulate expression of other genes encoding a protein, e.g., cytokines, e.g., decreasing CXCL expression and cellular recruitment of CXCL to the site of inflammation, reduces presence of GM-CSF, and/or IL-6 in the site of inflammation.

In some embodiments, a target plurality of genes is part of a genomic complex, e.g., ASMC. As used herein, referring to a target plurality of genes being part of a genomic complex, e.g., ASMC, means that each of the genes of the plurality are at least partly comprised within the genomic complex, e.g., ASMC. Referring to a target plurality of genes as part of a genomic complex, e.g., ASMC, is used interchangeably with reference to a genomic complex, e.g., ASMC, comprising a target plurality of genes. For example, a target plurality of genes may consist of two genes positioned adjacent one another in the genome wherein a first anchor sequence is disposed within the first of the genes and a second anchor sequence is disposed outside of the second of the genes distal to the first gene. An ASMC formed by association of said first and second anchor sequence would wholly comprise the second of the genes and partly comprise the first of the genes; a plurality of genes consisting of these two genes would be part of this ASMC. In some embodiments, each gene of a target plurality of genes is wholly within a genomic complex, e.g., ASMC, (e.g., no portion of the transcript encoding gene sequence is outside of the genomic complex, e.g., ASMC). In some embodiments, each gene of a target plurality of genes is partly within a genomic complex, e.g., ASMC, (e.g., some portion of the transcript encoding gene sequence is outside of the genomic complex, e.g., ASMC). In some embodiments, at least one gene of a target plurality of genes is wholly within a genomic complex, e.g., ASMC, (e.g., no portion of the transcript encoding gene sequence is outside of the genomic complex, e.g., ASMC), and at least one gene of a target plurality of genes is partly within a genomic complex, e.g., ASMC, (e.g., some portion of the transcript encoding gene sequence is outside of the genomic complex, e.g., ASMC).

A gene of a target plurality of genes may include coding sequences, e.g., exons, and/or non-coding sequences, e.g., introns, 3′UTR, or 5′UTR. In some embodiments, a gene of a target plurality of genes is operably linked to a genomic regulatory element (e.g., transcription control element). In some embodiments, a genomic regulatory element (e.g., transcription control element) of a gene of a target plurality of genes is also part of the genomic complex, e.g., ASMC, that the gene is part of. Referring to a genomic regulatory element (e.g., transcription control element) operably linked to a gene as part of a genomic complex, e.g., ASMC, can be understood in the same sense as described above in reference to the target plurality of genes. In some embodiments, each genomic regulatory element (e.g., transcription control element) operably linked to a gene of a target plurality of genes is wholly within a genomic complex, e.g., ASMC, (e.g., no portion of the genomic regulatory element (e.g., transcription control element) sequence is outside of the genomic complex, e.g., ASMC). In some embodiments, each genomic regulatory element (e.g., transcription control element) of a target plurality of genes is partly within a genomic complex, e.g., ASMC, (e.g., some portion of the genomic regulatory element (e.g., transcription control element) sequence is outside of the genomic complex, e.g., ASMC). In some embodiments, each genomic regulatory element (e.g., transcription control element) of a target plurality of genes is completely outside of the genomic complex, e.g., ASMC, (e.g., each genomic regulatory element (e.g., transcription control element) sequence is outside of the genomic complex, e.g., ASMC). In some embodiments, at least one genomic regulatory element (e.g., transcription control element) operably linked to a gene of a target plurality of genes is wholly within a genomic complex, e.g., ASMC (e.g., no portion of the genomic regulatory element (e.g., transcription control element) sequence is outside of the genomic complex, e.g., ASMC). In some embodiments, at least one genomic regulatory element (e.g., transcription control element) operably linked to another gene of the target plurality of genes is partly within the genomic complex, e.g., ASMC, (e.g., some portion of the transcript encoding gene sequence is outside of the genomic complex, e.g., ASMC). In some embodiments, at least one genomic regulatory element (e.g., transcription control element) operably linked to a gene of a target plurality of genes is completely outside of the genomic complex, e.g., ASMC.

In some embodiments, an expression repressor or a system targets a target plurality of genes by binding to a cRE (e.g., an E1 cRE) operably linked to the target plurality of genes. In some embodiments, a targeting moiety binds to the E1 cRE. In some embodiments, cRE can be disrupted to alter, e.g., inhibit, expression of a target plurality of genes.

A targeting moiety suitable for use in an expression repressor or a system may bind, e.g., specifically bind, to a site that is proximal to a cRE (e.g., a cRE operably linked to the plurality of genes, e.g., an E1 cRE). In some embodiments, a site proximal to a cRE sequence is a site that is less than 1000, 900, 800, 700, 600, 500, 400, 300, 200, 100, 50, 40, 30, 20, 10, or 5 base pairs from the cRE sequence (and optionally at least 5, 10, 20, 25, 50, 100, 200, or 300 base pairs). In some embodiments, a site proximal to a cRE sequence is a site that is less than 800, 700, 600, 500, 400, or 300 base pairs from the cRE sequence (and optionally at least 5, 10, 20, 25, 50, 100, 200, or 300 base pairs).

A targeting moiety suitable for use in an expression repressor or a system described herein may bind, e.g., specifically bind, to a site comprising at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs (and optionally no more 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 nucleotides or base pairs). In some embodiments, a targeting moiety binds to a site comprising 10, 11, 12, 13, 14, 15, 16, 17, 18, 19, 20, 21, 22, 23, 24, 25, 26, 27, 28, 29, 30, 31, 3233, 34, 35, 36, 37, 38, 39, 40, 41, 42, 43, 44, 45, 46, 47, 48, 49, or 50 nucleotides or base pairs.

In some embodiments, a site-specific disrupting agent or a system targets a target plurality of genes by binding to an anchor sequence, e.g., an anchor sequence that is part of a genomic complex (e.g., ASMC) comprising the target plurality of genes. In some embodiments, a targeting moiety binds to the anchor sequence. In some embodiments, binding of a genomic complex component, e.g., nucleating polypeptide, to an anchor sequence nucleates complex formation, e.g., anchor sequence-mediated conjunction formation. Each anchor sequence-mediated conjunction comprises one or more anchor sequences, e.g., a plurality of anchor sequences. In some embodiments, an anchor sequence-mediated conjunction can be disrupted to alter, e.g., inhibit, expression of a target plurality of genes. Such disruptions may modulate gene expression by, e.g., changing topological structure of DNA, e.g., by modulating the ability of a gene of the plurality to interact with a genomic regulatory element (e.g., transcription control element) (e.g., enhancing and silencing/repressive sequences).

A targeting moiety suitable for use in a site-specific disrupting agent or a system may bind, e.g., specifically bind, to a site that is proximal to an anchor sequence, e.g., an anchor sequence that is part of a genomic complex (e.g., ASMC) comprising the target plurality of genes. In some embodiments, a site proximal to a target gene (e.g., an exon, intron, or splice site within the target gene), proximal to a genomic regulatory element (e.g., transcription control element) operably linked to the target gene, or proximal to an anchor sequence is within 5000, 4000, 3000, 2000, 1000, 900, 800, 700, 600, 500, 400, 300, 200, 100, 50, 40, 30, 25, 20, 15, 10, or 5 base pairs from the target gene (e.g., an exon, intron, or splice site within the target gene), genomic regulatory element (e.g., transcription control element), or anchor sequence (and optionally at least 5, 10, 20, 25, 50, 100, 200, or 300 base pairs from the target gene (e.g., an exon, intron, or splice site within the target gene), genomic regulatory element (e.g., transcription control element), or anchor sequence). In some embodiments, a site proximal to an anchor sequence is a site that is less than 1000, 900, 800, 700, 600, 500, 400, 300, 200, 100, 50, 40, 30, 20, 10, or 5 base pairs from the anchor sequence (and optionally at least 5, 10, 20, 25, 50, 100, 200, or 300 base pairs). In some embodiments, a site proximal to an anchor sequence is a site that is less than 800, 700, 600, 500, 400, or 300 base pairs from the anchor sequence (and optionally at least 5, 10, 20, 25, 50, 100, 200, or 300 base pairs).

A targeting moiety suitable for use in a site-specific disrupting agent or a system described herein may bind, e.g., specifically bind, to a site comprising at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs (and optionally no more 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or nucleotides or base pairs). In some embodiments, a targeting moiety binds to a site comprising 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs.

### Genomic Complexes

Genomic complexes relevant to the present disclosure include stable structures that comprise a plurality of polypeptide and/or nucleic acid (particularly ribonucleic acid) components and that co-localize two or more genomic sequence elements (e.g., anchor sequences, promoter and/or enhancer elements). In some embodiments, genomic sequence elements that are (i.e., in three-dimensional space) in genomic complexes include transcriptional promoter and/or regulatory (e.g., enhancer or repressor) sequences. In some embodiments, relevant genomic complexes comprise anchor-sequence-mediated conjunctions (e.g., genomic loops). Alternatively, or additionally, in some embodiments, genomic sequence elements that are in genomic complexes include binding sites for one or more of CTCF, YY1, etc. In some embodiments, a genomic complex comprises a target plurality of genes. In some embodiments, two or more (e.g., all) genes of a target plurality of genes are located in a single loop of an ASMC. In some embodiments, two or more (e.g., all) genes of a target plurality of genes are located in different loops of an ASMC.

In some embodiments, a genomic complex whose incidence is decreased in accordance with the present disclosure comprises, or consists of, one or more components chosen from: a genomic sequence element (e.g., an anchor sequence, e.g., a CTCF binding motif, a YY1 binding motif, etc., that may, in some embodiments, be recognized by a nucleating component), one or more polypeptide components (e.g., one or more nucleating polypeptides, one or more transcriptional machinery proteins, and/or one or more transcriptional regulatory proteins), and/or one or more non-genomic nucleic acid components (e.g., non-coding RNA and/or an mRNA, for example, transcribed from a gene associated with the genomic complex).

In some embodiments, a genomic complex component is part of a genomic complex, wherein the genomic complex brings together two genomic sequence elements that are spaced apart from one another on a chromosome, e.g., via an interaction between and among a plurality of protein and/or other components.

In some embodiments, a genomic sequence element includes at least one or both of a promoter and/or regulatory site (e.g., an enhancer). In some embodiments, a genomic sequence element is an anchor sequences to which one or more protein components of the complex binds; thus, in some embodiments, a genomic complex comprises an anchor-sequence-mediated conjunction. In some embodiments, a genomic sequence element comprises a CTCF binding motif, a promoter and/or an enhancer. In some embodiments, complex formation is nucleated at the genomic sequence element(s) and/or by binding of one or more of the protein component(s) to the genomic sequence element(s).

Genomic sequence elements involved in genomic complexes as described herein, may be non-contiguous with one another. In some embodiments with noncontiguous genomic sequence elements (e.g., anchor sequences, promoters, and/or transcriptional regulatory sequences), a first genomic sequence element (e.g., anchor sequence, promoter, or transcriptional regulatory sequence) may be separated from a second genomic sequence element (e.g., anchor sequence, promoter, or transcriptional regulatory sequence) by about 500 bp to about 500 Mb, about 750 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a first genomic sequence element (e.g., anchor sequence, promoter, or transcriptional, regulatory sequence) is separated from a second genomic sequence element (e.g., anchor sequence, promoter, or transcriptional regulatory sequence) by about 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

### Anchor Sequence-Mediated Conjunction

In some embodiments, a genomic complex relevant to the present disclosure is or comprises an anchor sequence-mediated conjunction (ASMC). In some embodiments, an anchor-sequence-mediated conjunction is formed when nucleating polypeptide(s) bind to anchor sequences in the genome and interactions between and among these proteins and, optionally, one or more other components, forms a conjunction in which the anchor sequences are physically co-localized. In many embodiments described herein, one or more genes is associated with an anchor-sequence-mediated conjunction; in such embodiments, the anchor sequence-mediated conjunction typically includes one or more anchor sequences, one or more genes, and one or more transcriptional control sequences, such as an enhancing or silencing sequence. In some embodiments, a transcriptional control sequence is within, partially within, or outside an anchor sequence-mediated conjunction. In some embodiments, the ASMC comprises an internal enhancing sequence, e.g., an enhancer. In some embodiments, an ASMC comprises a target plurality of genes.

In some embodiments, a genomic complex as described herein (e.g., an anchor sequence-mediated conjunction) is or comprises a genomic loop, such as an intra-chromosomal loop. In certain embodiments, genomic complex as described herein (e.g., an anchor sequence-mediated conjunction) comprises a plurality of genomic loops. One or more genomic loops may include a first anchor sequence, a nucleic acid sequence, a transcriptional control sequence, and a second anchor sequence. In some embodiments, at least one genomic loop includes, in order, a first anchor sequence, a transcriptional control sequence, and a second anchor sequence; or a first anchor sequence, a nucleic acid sequence, and a second anchor sequence. In yet some embodiments, either one or both of nucleic acid sequences and transcriptional control sequence is located within a genomic loop. In yet some embodiments, either one or both of nucleic acid sequences and transcriptional control sequence is located outside a genomic loop. In some embodiments, one or more genomic loops comprise a transcriptional control sequence. In some embodiments, genomic complex (e.g., an anchor sequence-mediated conjunction) includes a TATA box, a CAAT box, a GC box, or a CAP site.

In some embodiments, an anchor sequence-mediated conjunction comprises a plurality of genomic loops; in some such embodiments, an anchor sequence-mediated conjunction comprises at least one of an anchor sequence, a nucleic acid sequence, and a transcriptional control sequence in one or more genomic loops.

### Types of Loops

In some embodiments, a genomic loop comprises one or more, e.g., 2, 3, 4, 5, or more, genes, e.g., a target plurality of genes. In some embodiments, two or more, e.g., 2, 3, 4, 5, or more, genes of the target plurality of genes are transcribed in the same direction. In some embodiments, all genes of the target plurality of genes are transcribed in the same direction.

In some embodiments, the present disclosure provides methods of modulating (e.g., decreasing) expression of a target plurality of genes in a loop comprising inhibiting, dissociating, degrading, and/or modifying a genomic complex that achieves co-localization of genomic sequences that are outside of, not part of, or comprised within (i) a gene whose expression is modulated (e.g. of a target plurality of genes); and/or (ii) one or more associated transcriptional control sequences that influence transcription of a gene whose expression is modulated.

In some embodiments, the present disclosure provides methods of modulating (e.g., decreasing) transcription of a target plurality of genes comprising inhibiting formation of and/or destabilizing a complex that achieves co-localization of genomic sequences that are non-contiguous with (i) a gene whose expression is modulated (e.g., of a target plurality of genes); and/or (ii) associated transcriptional control sequences that influence transcription of a gene whose expression is modulated.

In some embodiments, an anchor sequence-mediated conjunction is associated with one or more, e.g., 2, 3, 4, 5, or more, transcriptional control sequences. In some embodiments, a gene of a target plurality of genes (e.g., one, two, or more, e.g., all of the target plurality of genes) is non-contiguous with one or more transcriptional control sequences. In some embodiments where a gene is non-contiguous with its transcriptional control sequence(s), a gene may be separated from one or more transcriptional control sequences by about 100 bp to about 500 Mb, about 500 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a gene is separated from a transcriptional control sequence by about 100 bp, 300 bp, 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb. 500 Mb, or any size therebetween.

### Anchor Sequences

In general, an anchor sequence is a genomic sequence element to which a genomic complex component, e.g., nucleating polypeptide, binds specifically. In some embodiments, binding of a genomic complex component to an anchor sequence nucleates complex formation.

Each anchor sequence-mediated conjunction comprises one or more anchor sequences, e.g., a plurality. In some embodiments, anchor sequences can be manipulated or altered to form and/or stabilize naturally occurring loops, to form one or more new loops (e.g., to form exogenous loops or to form non-naturally occurring loops with exogenous or altered anchor sequences), or to inhibit formation of or destabilize naturally occurring or exogenous loops. Such alterations may modulate gene expression by, e.g., changing topological structure of DNA, e.g., by thereby modulating ability of a target gene to interact with gene regulation and control factors (e.g., enhancing and silencing/repressor sequences).

In some embodiments, chromatin structure is modified by substituting, adding or deleting one or more nucleotides within an anchor sequence-mediated conjunction. In some embodiments, chromatin structure is modified by substituting, adding, or deleting one or more nucleotides within an anchor sequence of an anchor sequence-mediated conjunction.

In some embodiments, an anchor sequence comprises a common nucleotide sequence, e.g., a CTCF-binding motif:
N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO: 1), where N is any nucleotide.

A CTCF-binding motif may also be in an opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO:2). In some embodiments, an anchor sequence comprises SEQ ID NO:1 or SEQ ID NO:2 or a sequence at least 75%, at least 80%, at least 90%, at least 95%, at least 96%, at least 97%, at least 98%, at least 99% identical to either SEQ ID NO:1 or SEQ ID NO:2.

In some embodiments, an anchor sequence comprises a CTCF binding motif, a USF1 binding motif, a YY1 binding motif, a TAF3 binding motif, or a ZNF143 binding motif. In some embodiments, an anchor sequence comprises a nucleating polypeptide binding motif, e.g., a YY1-binding motif: CCGCCATNTT, where N is any nucleotide. A YY1-binding motif may also be in an opposite orientation, e.g., AANATGGCGG, where N is any nucleotide. In some embodiments, an anchor sequence comprises CCGCCATNTT or AANATGGCGG or a sequence at least 75%, at least 80%, at least 90%, at least 95%, at least 96%, at least 97%, at least 98%, at least 99% identical to either CCGCCATNTT or AANATGGCGG.

In some embodiments, an anchor sequence-mediated conjunction comprises at least a first anchor sequence and a second anchor sequence. For example, in some embodiments, a first anchor sequence and a second anchor sequence may each comprise a common nucleotide sequence, e.g., each comprises a CTCF binding motif. In some embodiments, a first anchor sequence and a second anchor sequence may each comprise a USF1 binding motif. In some embodiments, a first anchor sequence and a second anchor sequence may each comprise a YY1 binding motif. In some embodiments, a first anchor sequence and a second anchor sequence may each comprise a TAF3 binding motif. In some embodiments, a first anchor sequence and a second anchor sequence may each comprise a ZNF143 binding motif.

In some embodiments, a first anchor sequence and second anchor sequence comprise different sequences, e.g., a first anchor sequence comprises a CTCF binding motif, and a second anchor sequence comprises an anchor sequence other than a CTCF binding motif. In some embodiments, a first anchor sequence comprises a CTCF binding motif, a USF1 binding motif, a YY1 binding motif, a TAF3 binding motif, or a ZNF143 binding motif, and a second anchor sequence comprises a CTCF binding motif, a USF1 binding motif, a YY1 binding motif, a TAF3 binding motif, or a ZNF143 binding motif, wherein the first and second anchor sequences do not both comprise a CTCF binding motif, a USF1 binding motif, a YY1 binding motif, a TAF3 binding motif, or a ZNF143 binding motif. In some embodiments, each anchor sequence comprises a common nucleotide sequence (e.g., a CTCF binding motif, a USF1 binding motif, a YY1 binding motif, a TAF3 binding motif, or a ZNF143 binding motif) and one or more flanking nucleotides on one or both sides of a common nucleotide sequence.

Two anchor sequences (e.g., each comprising a CTCF binding motif, a USF1 binding motif, a YY1 binding motif, a TAF3 binding motif, or a ZNF143 binding motif) that can form a conjunction may be present in a genome in any orientation, e.g., in the same orientation (tandem) either 5′-3′ (left tandem, or 3′-5′ (right tandem), or convergent orientation, where one anchor sequence is oriented 5′-3′ and the other is oriented 3′-5′.

Two CTCF-binding motifs (e.g., contiguous or non-contiguous CTCF binding motifs) that can form a conjunction may be present in a genome in any orientation, e.g., in the same orientation (tandem) either 5′-3′ (left tandem, e.g., the two CTCF-binding motifs that comprise SEQ ID NO:1) or 3′-5′ (right tandem, e.g., the two CTCF-binding motifs comprise SEQ ID NO:2), or convergent orientation, where one CTCF-binding motif comprises SEQ ID NO:1 and another other comprises SEQ ID NO:2. CTCFBSDB 2.0: Database For CTCF binding motifs And Genome Organization (on the world wide web at insulatordb.uthsc.edu/) can be used to identify CTCF binding motifs associated with a target gene.

In some embodiments, an anchor sequence comprises a CTCF binding motif associated with a target plurality of genes, wherein the target plurality of genes is associated with a disease, disorder and/or condition. In some embodiments, an anchor sequence comprises a CTCF binding motif associated with a target plurality of genes, wherein the genes of the target plurality of genes have related functionalities. In some embodiments, an anchor sequence comprises a CTCF binding motif associated with a target plurality of genes, wherein the target plurality of genes (e.g., two or more, e.g., all, of the plurality) are aberrantly expressed in a cell of a subject.

In some embodiments, chromatin structure may be modified by substituting, adding, or deleting one or more nucleotides within at least one anchor sequence, e.g., a nucleating polypeptide binding motif. One or more nucleotides may be specifically targeted, e.g., a targeted alteration, for substitution, addition or deletion within an anchor sequence, e.g., a nucleating polypeptide binding motif.

In some embodiments, an anchor sequence-mediated conjunction may be altered by changing an orientation of at least one common nucleotide sequence, e.g., a nucleating polypeptide binding motif. In some embodiments, an anchor sequence comprises a nucleating polypeptide binding motif, e.g., CTCF binding motif, and a targeting moiety introduces an alteration in at least one nucleating polypeptide binding motif, e.g., altering binding affinity for a nucleating polypeptide.

In some embodiments, an anchor sequence-mediated conjunction may be altered by introducing an exogenous anchor sequence. In some embodiments, addition of a non-naturally occurring or exogenous anchor sequence to destabilize or inhibit formation of a naturally occurring anchor sequence-mediated conjunction, e.g., by inducing a non-naturally occurring loop to form, alters (e.g., decreases) transcription of a nucleic acid sequence.

### Other Compositions

### Nucleic Acids and Vectors

The present disclosure is further directed, in part, to nucleic acids encoding an expression repressor and/or a site-specific disrupting agent or a system described herein.

In some embodiments, an expression repressor may be provided via a composition comprising a nucleic acid encoding an expression repressor, e.g., a targeting moiety and/or effector moiety of the expression repressor, wherein the nucleic acid is associated with sufficient other sequences to achieve expression of the expression repressor in a system of interest (e.g., in a particular cell, tissue, organism, etc). In some embodiments, system may be provided via a composition comprising a first nucleic acid encoding a first expression repressor, e.g., a first targeting moiety and/or a first effector moiety of the first expression repressor, and a second nucleic acid encoding a second expression repressor, e.g., a second targeting moiety and/or a second effector moiety of the second expression repressor wherein the first and/or the second nucleic acid is associated with sufficient other sequences to achieve expression of the expression repressor in a system of interest (e.g., in a particular cell, tissue, organism, etc). In some embodiments, system may be provided via a composition comprising a first nucleic acid encoding a first expression repressor, e.g., a first targeting moiety and/or a first effector moiety of the first expression repressor, and a second nucleic acid encoding a site-specific disrupting agent, e.g., a targeting moiety and/or an effector moiety of the site-specific disrupting agent wherein the first and/or the second nucleic acid is associated with sufficient other sequences to achieve expression of the expression repressor and the site-specific disrupting agent in a system of interest (e.g., in a particular cell, tissue, organism, etc). In some embodiments, system may be provided via a composition comprising a first nucleic acid encoding a first expression repressor, e.g., a first targeting moiety and/or a first effector moiety of the first expression repressor, a second nucleic acid encoding second expression repressor, e.g., a second targeting moiety and/or a second effector moiety of the second expression repressor, and a third nucleic acid encoding a site-specific disrupting agent, e.g., a targeting moiety and/or an effector moiety of the site-specific disrupting agent wherein the first, the second, and/or the third nucleic acid is associated with sufficient other sequences to achieve expression of the expression repressors and the site-specific disrupting agent in a system of interest (e.g., in a particular cell, tissue, organism, etc).

In some embodiments, the present disclosure provides compositions of nucleic acids that encode an expression repressor or polypeptide or nucleic acid portion thereof (e.g., a targeting moiety and/or effector moiety comprising a polypeptide and/or nucleic acid). In some embodiments, the present disclosure provides compositions of nucleic acids that encode a first expression repressor and a second expression repressor, or polypeptides or nucleic acid portions thereof (e.g., a targeting moiety and/or effector moiety comprising a polypeptide and/or nucleic acid). In some such embodiments, provided nucleic acids may include DNA, RNA, or any other nucleic acid moiety or entity as described herein, and may be prepared by any suitable technology, e.g., a technology described herein or otherwise available in the art (e.g., synthesis, cloning, amplification, in vitro or in vivo transcription, etc). In some embodiments, provided nucleic acids that encode one or more expression repressors, or polypeptides or nucleic acid portions thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc.).

In some embodiments, a site-specific disrupting agent may be provided via a composition comprising a nucleic acid encoding a site-specific disrupting agent, e.g., a targeting moiety and/or effector moiety of the site-specific disrupting agent, wherein the nucleic acid is associated with sufficient other sequences to achieve expression of the site-specific disrupting agent in a system of interest (e.g., in a particular cell, tissue, organism, etc). In some embodiments, system may be provided via a composition comprising a first nucleic acid encoding a first site-specific disrupting agent, e.g., a first targeting moiety and/or a first effector moiety of the first site-specific disrupting agent, and a second nucleic acid encoding a second site-specific disrupting agent, e.g., a second targeting moiety and/or a second effector moiety of the second site-specific disrupting agent wherein the first and/or the second nucleic acid is associated with sufficient other sequences to achieve expression of the site-specific disrupting agents in a system of interest (e.g., in a particular cell, tissue, organism, etc).

In some particular embodiments, the present disclosure provides compositions of nucleic acids that encode a site-specific disrupting agent or polypeptide or nucleic acid portion thereof (e.g., a targeting moiety and/or effector moiety comprising a polypeptide and/or nucleic acid). In some particular embodiments, the present disclosure provides compositions of nucleic acids that encode a first site-specific disrupting agent and a second site-specific disrupting agent, or polypeptides or nucleic acid portions thereof (e.g., a targeting moiety and/or effector moiety comprising a polypeptide and/or nucleic acid). In some such embodiments, provided nucleic acids may include DNA, RNA, or any other nucleic acid moiety or entity as described herein, and may be prepared by any suitable technology, e.g., a technology described herein or otherwise available in the art (e.g., synthesis, cloning, amplification, in vitro or in vivo transcription, etc). In some embodiments, provided nucleic acids that encode one or more site-specific disrupting agents, or polypeptides or nucleic acid portions thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc.).

In some embodiments, a composition for delivering an expression repressor and/or a site-specific disrupting agent or a system described herein comprises a vector, e.g., a viral vector, comprising one or more nucleic acids encoding an expression repressor and/or a site-specific disrupting agent or polypeptide or nucleic acid portion thereof. In some embodiments, a first vector comprises a first nucleic acid encoding a first expression repressor, and second vector comprises a second nucleic acid encoding a second expression repressor. In some embodiments a single vector comprises a first nucleic acid encoding a first expression repressor and a second nucleic acid encoding a second expression repressor. In some embodiments, a first vector comprises a first nucleic acid encoding an expression repressor, and second vector comprises a second nucleic acid encoding a site-specific disrupting agent. In some embodiments a single vector comprises a first nucleic acid encoding an expression repressor and a second nucleic acid encoding a site-specific disrupting agent. In some embodiments, a first vector comprises a first nucleic acid encoding a first site-specific disrupting agent, and second vector comprises a second nucleic acid encoding a second site-specific disrupting agent. In some embodiments a single vector comprises a first nucleic acid encoding a first site-specific disrupting agent and a second nucleic acid encoding a second site-specific disrupting agent.

In some embodiments, a composition for delivering an expression repressor and/or a site-specific disrupting agent or a system described herein is or comprises RNA, e.g., mRNA, comprising one or more nucleic acids encoding one or more components of an expression repressor or polypeptide or nucleic acid portion thereof and/or a site-specific disrupting agent or polypeptide or nucleic acid portion thereof.

Nucleic acids as described herein or nucleic acids encoding a protein described herein, may be incorporated into a vector. Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. An expression vector may be provided to a cell in the form of a viral vector. Viral vector technology is well known in the art and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Expression of natural or synthetic nucleic acids is typically achieved by operably linking a nucleic acid encoding the gene of interest to a promoter and incorporating the construct into an expression vector. Vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, may regulate frequency of transcriptional initiation. Typically, these sequences are located in a region 30-110 bp upstream of a transcription start site, although a number of promoters have recently been shown to contain functional elements downstream of transcription start sites as well. Spacing between promoter elements frequently is flexible, so that promoter function is preserved when elements are inverted or moved relative to one another. In a thymidine kinase (tk) promoter, spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. In some embodiments of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, an actin promoter, a myosin promoter, a hemoglobin promoter, and a creatine kinase promoter.

The present disclosure should not be interpreted to be limited to use of any particular promoter or category of promoters (e.g., constitutive promoters). For example, in some embodiments, inducible promoters are contemplated as part of the present disclosure. In some embodiments, use of an inducible promoter provides a molecular switch capable of turning on expression of a polynucleotide sequence to which it is operatively linked, when such expression is desired. In some embodiments, use of an inducible promoter provides a molecular switch capable of turning off expression when expression is not desired. Examples of inducible promoters include, but are not limited to a metallothionine promoter, a glucocorticoid promoter, a progesterone promoter, and a tetracycline promoter.

In some embodiments, an expression vector to be introduced can also contain either a selectable marker gene or a reporter gene or both to facilitate identification and selection of expressing cells from the population of cells sought to be transfected or infected through viral vectors. In some aspects, a selectable marker may be carried on a separate piece of DNA and used in a co-transfection procedure. Both selectable markers and reporter genes may be flanked with appropriate transcriptional control sequences to enable expression in the host cells. Useful selectable markers may include, for example, antibiotic-resistance genes, such as neo, etc.

In some embodiments, reporter genes may be used for identifying potentially transfected cells and/or for evaluating the functionality of transcriptional control sequences. In general, a reporter gene is a gene that is not present in or expressed by a recipient source (of a reporter gene) and that encodes a polypeptide whose expression is manifested by some easily detectable property, e.g., enzymatic activity or visualizable fluorescence. Expression of a reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, a construct with a minimal 5′ flanking region that shows highest level of expression of reporter gene is identified as a promoter. Such promoter regions may be linked to a reporter gene and used to evaluate agents for ability to modulate promoter-driven transcription.

### Cells

The present disclosure is further directed, in part, to cells comprising an expression repressor and/or site-specific disrupting agent, or a system described herein. Any cell, e.g., cell line. e.g., a cell line suitable for expression of a recombinant polypeptide, known to one of skill in the art is suitable to comprise an expression repressor and/or a site-specific disrupting agent described herein. In some embodiments, a cell, e.g., cell line, may be used to express an expression repressor and/or a site-specific disrupting agent, a system comprising one or more expression repressors and/or one or more site-specific disrupting agents, or nucleic acid or polypeptide portion thereof. In some embodiments, a cell, e.g., cell line, may be used to express or amplify a nucleic acid, e.g., a vector, encoding an expression repressor and/or a site-specific disrupting agent. In some embodiments, a cell, e.g., cell line, may be used to express or amplify one or more nucleic acids, e.g., a vector, encoding a first expression repressor and a vector encoding a second expression repressor. In some embodiments, a cell, e.g., cell line, may be used to express or amplify one or more nucleic acids, e.g., a vector, encoding an expression repressor and a vector encoding a site specific disrupting agent. In some embodiments, a cell, e.g., cell line, may be used to express or amplify one or more nucleic acids, e.g., a vector, encoding a first site-specific disrupting agent and a vector encoding a second site specific disrupting agent. In some embodiments, a cell comprises a nucleic acid encoding an expression repressor described herein. In some embodiments, a cell comprises a nucleic acid encoding a site-specific disrupting agent described herein. In some embodiments, a cell comprises a first nucleic acid encoding a first expression repressor and a second nucleic acid encoding a second expression repressor described herein. In some embodiments, a cell comprises a first nucleic acid encoding an expression repressor and a second nucleic acid encoding a site-specific disrupting agent described herein. In some embodiments, a cell comprises a first nucleic acid encoding a first site-specific disrupting agent and a second nucleic acid encoding a second site-specific disrupting agent described herein.

In some embodiments, a cell comprises a nucleic acid encoding an expression repressor and/or a site-specific disrupting agent or nucleic acid or polypeptide portion thereof, and the nucleic acid is integrated into the genomic DNA of the cell. In some embodiments, a cell comprises a nucleic acid encoding an expression repressor and/or a site-specific disrupting agent or nucleic acid or polypeptide portion thereof, and the nucleic acid is disposed on a vector. In some embodiments, a cell comprises a first nucleic acid encoding a first expression repressor and a second nucleic acid encoding a second expression repressor or nucleic acid or polypeptide portion thereof, and the first and the second nucleic acid are integrated into the genomic DNA of the cell. In some embodiments, a cell comprises a first nucleic acid encoding an expression repressor and a second nucleic acid encoding a site-specific disrupting agent or nucleic acid or polypeptide portion thereof, and the first and the second nucleic acid are integrated into the genomic DNA of the cell. In some embodiments, a cell comprises a first nucleic acid encoding a first expression repressor and a second nucleic acid encoding a second expression repressor or nucleic acid or polypeptide portion thereof, and the first and the second nucleic acid are disposed on a vector. In some embodiments, a cell comprises a first nucleic acid encoding an expression repressor and a second nucleic acid encoding a site-specific disrupting agent or nucleic acid or polypeptide portion thereof, and the first and the second nucleic acid are disposed on a vector. In some embodiments, a cell comprises a first nucleic acid encoding a first site-specific disrupting agent and a second nucleic acid encoding a second site-specific disrupting agent or nucleic acid or polypeptide portion thereof, and the first and the second nucleic acid are integrated into the genomic DNA of the cell. In some embodiments, a cell comprises a first nucleic acid encoding a first site-specific disrupting agent and a second nucleic acid encoding a second site-specific disrupting agent or nucleic acid or polypeptide portion thereof, and the first and the second nucleic acid are disposed on a vector.

Examples of cells that may comprise and/or express an expression repressor and/or a site-specific disrupting agent, or a system described herein include, but are not limited to, hepatocytes, stellate cells, Kupffer cells, neuronal cells, endothelial cells, alveolar cells, epithelial cells, myocytes, synovial layer, chondrocytes, immune cells, and lymphocytes.

The present disclosure is further directed, in part, to a cell made by a method or process described herein. In some embodiments, the disclosure provides a cell produced by, providing an expression repressor described herein, providing the cell, and contacting the cell with the expression repressor (or a nucleic acid encoding the expression repressor, or a composition comprising said expression repressor or nucleic acid). In some embodiments, the disclosure provides a cell produced by, providing a system described herein, providing the cell, and contacting the cell with the system (or a first nucleic acid encoding the first expression repressor and second nucleic acid encoding the second expression repressor, or a composition comprising said system or nucleic acids). In some embodiments, the disclosure provides a cell produced by, providing a system described herein, providing the cell, and contacting the cell with the system (or a first nucleic acid encoding the expression repressor and second nucleic acid encoding the site-specific disrupting agent, or a composition comprising said system or nucleic acids). Without wishing to be bound by theory, a cell contacted with an expression repressor or a system described herein may exhibit: a decrease in expression of a target plurality of genes; a modification of epigenetic markers associated with the target plurality of genes, a genomic regulatory element (e.g., transcription control element) operably linked to the target plurality of genes, or an anchor sequence proximal to the target plurality of genes or associated with a cis-acting regulatory element (cRE, e.g., E1 cRE) operably linked to the target plurality of genes; a genetic modification of a gene of the target plurality of genes, a genomic regulatory element (e.g., transcription control element) operably linked to the target plurality of genes, or an anchor sequence proximal to the target plurality of genes or associated with an anchor sequence-mediated conjunction comprising the target plurality of genes; and/or a decrease (e.g., the absence of) in the level of a genomic complex, e.g., ASMC, comprising a target plurality of genes, compared to a similar cell that has not been contacted by the expression repressor or a system described herein. In some embodiments, a cell exhibiting said decrease in expression of a target plurality of genes, modification of epigenetic markers, and/or genetic modification does not comprise the expression repressor or a system described herein. The decrease in expression of a target plurality of genes, modification of epigenetic markers, and/or genetic modification may persist, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after contact with the expression repressor of system described herein. In some embodiments, a cell previously contacted by an expression repressor or system described herein retains the decrease in expression of a target plurality of genes, modification of epigenetic markers, and/or genetic modification after the expression repressor or system described herein is no longer present in the cell, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after the expression repressor or system described herein is no longer present in the cell. In some embodiments, the cell is a mammalian, e.g., human, cell. In some embodiments, the cell is a somatic cell and/or a primary cell.

The present disclosure is further directed, in part, to a cell made by a method or process described herein. In some embodiments, the disclosure provides a cell produced by, providing a site-specific disrupting agent described herein, providing the cell, and contacting the cell with the site-specific disrupting agent (or a nucleic acid encoding the site-specific disrupting agent, or a composition comprising said site-specific disrupting agent or nucleic acid). In some embodiments, the disclosure provides a cell produced by, providing system described herein, providing the cell, and contacting the cell with the system (or a first nucleic acid encoding the first site-specific disrupting agent and second nucleic acid encoding the second site-specific disrupting agent, or a composition comprising said system or nucleic acids). Without wishing to be bound by theory, a cell contacted with a site-specific disrupting agent or a system described herein may exhibit: a decrease in expression of a target plurality of genes; a modification of epigenetic markers associated with the target plurality of genes, a genomic regulatory element (e.g., transcription control element) operably linked to the target plurality of genes, or an anchor sequence proximal to the target plurality of genes or associated with an anchor sequence-mediated conjunction comprising the target plurality of genes; a genetic modification of a gene of the target plurality of genes, a genomic regulatory element (e.g., transcription control element) operably linked to the target plurality of genes, or an anchor sequence proximal to the target plurality of genes or associated with an anchor sequence-mediated conjunction comprising the target plurality of genes; and/or a decrease (e.g., the absence of) in the level of a genomic complex, e.g., ASMC, comprising a target plurality of genes, compared to a similar cell that has not been contacted by the site-specific disrupting agent. In some embodiments, a cell exhibiting said decrease in expression of a target plurality of genes, modification of epigenetic markers, and/or genetic modification does not comprise the site-specific disrupting agent. The decrease in expression of a target plurality of genes, modification of epigenetic markers, and/or genetic modification may persist, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after contact with the site-specific disrupting agent. In some embodiments, a cell previously contacted by a site-specific disrupting agent retains the decrease in expression of a target plurality of genes, modification of epigenetic markers, and/or genetic modification after the site-specific disrupting agent is no longer present in the cell, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after the site-specific disrupting agent is no longer present in the cell. In some embodiments, the cell is a mammalian, e.g., human, cell. In some embodiments, the cell is a somatic cell and/or a primary cell.

### Kits

The present disclosure further directed, in part, to a kit comprising an expression repressor, a system, nucleic acid encoding an expression repressor, a first nucleic acid encoding the first expression repressor and a second nucleic acid encoding the second expression repressor, or a first nucleic acid encoding the expression repressor and a second nucleic acid encoding the site-specific disrupting agent described herein. In some embodiments, a kit comprises an expression repressor, a system, or nucleic acid encoding the same and instructions for the use of said expression repressor or the system. In some embodiments, a kit comprises a nucleic acid encoding the expression repressor or a component thereof (e.g., a polypeptide or nucleic acid portion of the expression repressor) and instructions for the use of said nucleic acid and/or said expression repressor. In some embodiments, a kit comprises a cell comprising a nucleic acid encoding the expression repressor or a component thereof (e.g., a polypeptide or nucleic acid portion of the expression repressor) and instructions for the use of said cell, nucleic acid, and/or said expression repressor. In some embodiments, a kit comprises or a first nucleic acid encoding the first expression repressor and a second nucleic acid encoding the second expression repressor or a component thereof (e.g., a polypeptide or nucleic acid portion of the first and the second expression repressor) and instructions for the use of said nucleic acids and/or said system. In some embodiments, a kit comprises a cell comprising a nucleic acid encoding or a first nucleic acid encoding the first expression repressor and a second nucleic acid encoding the second expression repressor or a component thereof (e.g., a polypeptide or nucleic acid portion of the first and the second expression repressor) and instructions for the use of said cell, nucleic acid, and/or said system. In some embodiments, a kit comprises or a first nucleic acid encoding the expression repressor and a second nucleic acid encoding the site-specific disrupting agent or a component thereof (e.g., a polypeptide or nucleic acid portion of the first expression repressor and the site-specific disrupting agent) and instructions for the use of said nucleic acids and/or said system. In some embodiments, a kit comprises a cell comprising a nucleic acid encoding or a first nucleic acid encoding the first site-specific disrupting agent and a second nucleic acid encoding the second site-specific disrupting agent or a component thereof (e.g., a polypeptide or nucleic acid portion of the first and the second site-specific disrupting agent) and instructions for the use of said cell, nucleic acid, and/or said system.

In some embodiments, a kit comprises a unit dosage of an expression repressor, or a unit dosage of a nucleic acid, e.g., a vector, encoding an expression repressor described herein. In some embodiments, a kit comprises a unit dosage of a system, or a unit dosage of a first and a second nucleic acid, e.g., a vector, encoding a first expression repressor and a second expression repressor described herein. In some embodiments, a kit comprises a unit dosage of a system, or a unit dosage of a first and a second nucleic acid, e.g., a vector, encoding an expression repressor and a site-specific disrupting agent described herein.

The present disclosure further directed, in part, to a kit comprising a site-specific disrupting agent, a system, nucleic acid encoding a site-specific disrupting agent, or a first nucleic acid encoding the first site-specific disrupting agent and a second nucleic acid encoding the second site-specific disrupting agent described herein. In some embodiments, a kit comprises a site-specific disrupting agent, a system, or nucleic acid encoding the same and instructions for the use of said site-specific disrupting agent or the system. In some embodiments, a kit comprises a nucleic acid encoding the site-specific disrupting agent or a component thereof (e.g., a polypeptide or nucleic acid portion of the site-specific disrupting agent) and instructions for the use of said nucleic acid and/or said site-specific disrupting agent. In some embodiments, a kit comprises a cell comprising a nucleic acid encoding the site-specific disrupting agent or a component thereof (e.g., a polypeptide or nucleic acid portion of the site-specific disrupting agent) and instructions for the use of said cell, nucleic acid, and/or said site-specific disrupting agent. In some embodiments, a kit comprises or a first nucleic acid encoding the first site-specific disrupting agent and a second nucleic acid encoding the second site-specific disrupting agent or a component thereof (e.g., a polypeptide or nucleic acid portion of the first and the second site-specific disrupting agent) and instructions for the use of said nucleic acids and/or said system. In some embodiments, a kit comprises a cell comprising a nucleic acid encoding or a first nucleic acid encoding the first site-specific disrupting agent and a second nucleic acid encoding the second site-specific disrupting agent or a component thereof (e.g., a polypeptide or nucleic acid portion of the first and the second site-specific disrupting agent) and instructions for the use of said cell, nucleic acid, and/or said system. In some embodiments, a kit comprises a unit dosage of a site-specific disrupting agent, or a unit dosage of a nucleic acid, e.g., a vector, encoding a site-specific disrupting agent described herein. In some embodiments, a kit comprises a unit dosage of a system, or a unit dosage of a first and a second nucleic acid, e.g., a vector, encoding a first site-specific disrupting agent and a second site-specific disrupting agent described herein.

### Methods of Making an Expression Repressor or Site-Specific Disrupting Agent

In some embodiments, an expression repressor, a site-specific disrupting agent, or a system comprises one or more proteins and may thus be produced by methods of making proteins or nucleic acids. As will be appreciated by one of skill, methods of making proteins or polypeptides (which may be included in modulating agents as described herein) are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

A protein or polypeptide of compositions of the present disclosure can be biochemically synthesized by employing standard solid phase techniques. Such methods include exclusive solid phase synthesis, partial solid phase synthesis methods, fragment condensation, classical solution synthesis. These methods can be used when a peptide is relatively short (e.g., 10 kDa) and/or when it cannot be produced by recombinant techniques (i.e., not encoded by a nucleic acid sequence) and therefore involves different chemistry.

Solid phase synthesis procedures are well known in the art and further described by John Morrow Stewart and Janis Dillaha Young, Solid Phase Peptide Syntheses, 2nd Ed., Pierce Chemical Company, 1984; and Coin, I., et al., Nature Protocols, 2:3247-3256, 2007.

For longer peptides, recombinant methods may be used. Methods of making a recombinant therapeutic polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Exemplary methods for producing a therapeutic pharmaceutical protein or polypeptide involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In cases where large amounts of the protein or polypeptide are desired, it can be generated using techniques such as described by Brian Bray, Nature Reviews Drug Discovery, 2:587-593, 2003; and Weissbach & Weissbach, 1988, Methods for Plant Molecular Biology, Academic Press, NY, Section VIII, pp 421-463.

Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012).

Proteins comprise one or more amino acids. Amino acids include any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

### Pharmaceutical Compositions, Formulation, Delivery, and Administration

The present disclosure is further directed, in part, to pharmaceutical compositions comprising an expression repressor and/or a site-specific disrupting agent described herein, and to pharmaceutical compositions comprising nucleic acids encoding an expression repressor and/or a site-specific disrupting agent or a system described herein.

As used herein, the term “pharmaceutical composition” refers to an active agent (e.g., an expression repressor and/or a site-specific disrupting agent, or a system, or nucleic acid encoding the same), formulated together with one or more pharmaceutically acceptable carriers (e.g., pharmaceutically acceptable carriers known to those of skill in the art). In some embodiments, active agent is present in unit dose amount appropriate for administration in a therapeutic regimen that shows a statistically significant probability of achieving a predetermined therapeutic effect when administered to a relevant population. In some embodiments, a pharmaceutical composition comprises an expression repressor or a system of the present disclosure. In some embodiments, a pharmaceutical composition comprises an expression repressor and a site-specific disrupting agent, or a system of the present disclosure. In some embodiments, a pharmaceutical composition comprises a site-specific disrupting agent or a system of the present disclosure.

In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; trans-dermally; or nasally, pulmonary, and/or to other mucosal surfaces, for example, as aerosols, aqueous solutions, or suspensions. In some embodiments, the composition may be lyophilized or spray dried. In some embodiments, the composition may be formulated for pulmonary administration and/or intravenous administration.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In some embodiments, for example, materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt”, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid, or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate, and aryl sulfonate.

In various embodiments, the present disclosure provides pharmaceutical compositions described herein with a pharmaceutically acceptable excipient. Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutical preparations may be made following conventional techniques of pharmacy involving milling, mixing, granulation, and compressing, when necessary, for tablet forms; or milling, mixing, and filling for hard gelatin capsule forms. When a liquid carrier is used, a preparation can be in the form of a syrup, elixir, emulsion or an aqueous or non-aqueous solution or suspension. Such a liquid formulation may be administered directly per os.

In some embodiments, pharmaceutical compositions may be formulated for delivery to a cell and/or to a subject via any route of administration. Modes of administration to a subject may include injection, infusion, inhalation, intranasal, intraocular, topical delivery, inter-cannular delivery, or ingestion. Injection includes, without limitation, intravenous, intramuscular, intra-arterial, intrathecal, intraventricular, intracapsular, intra-orbital, intracardiac, intradermal, intraperitoneal, transtracheal, subcutaneous, subcuticular, intraarticular, bronchial, sub-capsular, subarachnoid, intraspinal, intra-cerebrospinal, and intra-sternal injection and infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), or local (e.g., local application on the skin, intravitreal injection). In some embodiments, one or more compositions is administered systemically. In some embodiments, administration is non-parenteral and a therapeutic is a parenteral therapeutic. In some embodiments, administration may be bronchial (e.g., by bronchial instillation), buccal, dermal (which may be or comprise, for example, one or more of topical to the dermis, intradermal, inter-dermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be a single dose. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, six, eight, ten, 12, 15 or 20 or more administrations may be given to the subject during one treatment or over a period of time as a treatment regimen. In some embodiments, administrations may be given as needed, e.g., for as long as symptoms associated with the disease, disorder or condition persist. In some embodiments, repeated administrations may be indicated for the remainder of the subject's life. Treatment periods may vary and could be, e.g., one day, two days, three days, one week, two weeks, one month, two months, three months, six months, a year, or longer.

In some embodiments, administration is provided using a respiratory delivery device, e.g., nebulizer, e.g., metered-dose inhaler, e.g., dry powder inhaler. Some of the commercially available dry powder inhalers include Spinhaler (Fisons Pharmaceuticals, Rochester, NY) and Rotahaler (GSK, RTP, NC). In some embodiments, the nebulizer may include a jet nebulizer, an ultrasonic nebulizer, and/or a vibrating mesh nebulizer.

### Dosing Regimen

In methods of the inventions, the nucleic acid encoding an expression repressor may be administered according to a defined dosing regimen. The dosing regimen may include a defined dose, a defined interval between doses, a defined period of dosing, or any combination thereof. The dosing regimen can vary based on, e.g., the condition being treated, the severity of the disease, the subject's individual parameters, including age, physiological condition, size and weight, duration of treatment, the type of treatment to be performed (if any), the particular route of administration and similar factors. Thus, the dosing regimen of the agents described herein can depend on such various parameters. The dosing regimen of an administered composition may also vary depending upon other factors as the subject's sex, general medical condition, and severity of the disorder to be treated.

Pharmaceutical compositions according to the present disclosure may be delivered in a therapeutically effective amount. A precise therapeutically effective amount is an amount of a composition that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

In some aspects, the present disclosure provides methods of delivering a therapeutic comprising administering a composition as described herein to a subject, wherein an expression repressor is a therapeutic and/or wherein delivery of a therapeutic causes changes in gene expression relative to gene expression in absence of a therapeutic.

In some aspects, the present disclosure provides methods of delivering a therapeutic comprising administering a composition as described herein to a subject, wherein a genomic complex modulating agent is a therapeutic and/or wherein delivery of a therapeutic causes changes in gene expression relative to gene expression in absence of a therapeutic.

Methods as provided in various embodiments herein may be utilized in any some aspects delineated herein. In some embodiments, one or more compositions is/are targeted to specific cells, or one or more specific tissues.

For example, in some embodiments one or more compositions is/are targeted to epithelial, connective, muscular, and/or nervous tissue or cells. In some embodiments a composition is targeted to a cell or tissue of a particular organ system, e.g., respiratory system (pharynx, larynx, trachea, bronchi, lungs, diaphragm), cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); skeletal system (bone, cartilage); and/or combinations thereof. In some embodiments, a composition is targeted to a cell, e.g., endothelial, alveolar, epithelial, hepatocytes, stellate cells, Kupffer cells, synovial layer, chondrocytes, fibroblast cells, ductal epithelial cells, epithelial enterocytes, goblet cells, basal cells, and/or immune cells. In some embodiments, a composition is targeted to a cell of an organ, e.g., nasal cells, lung cells, ileum cells, cardiac cells, optic cells, liver cells, bladder cells, pancreatic cells, kidney cells, neural cells, prostrate cells, or testis cells.

In some embodiments, a composition of the present disclosure crosses a blood-brain-barrier, a placental membrane, or a blood-testis barrier. In some embodiments, a composition is targeted to a cell expressing an ACE-2 receptor.

In some embodiments, a pharmaceutical composition as provided herein is administered systemically.

In some embodiments, administration is non-parenteral and a therapeutic is a parenteral therapeutic.

In some embodiments, a pharmaceutical composition of the present disclosure has improved PK/PD, e.g., increased pharmacokinetics or pharmacodynamics, such as improved targeting, absorption, or transport (e.g., at least 5%, 10%, 15%, 20%, 30%, 40%, 50%, 60%, 75%, 80%, 90% improved or more) as compared to an active agent alone. In some embodiments, a pharmaceutical composition has reduced undesirable effects, such as reduced diffusion to a nontarget location, off-target activity, or toxic metabolism, as compared to a therapeutic alone (e.g., at least 5%, 10%, 15%, 20%, 30%, 40%, 50%, 60%, 75%, 80%, 90% or more reduced, as compared to an active agent alone). In some embodiments, a composition increases efficacy and/or decreases toxicity of a therapeutic (e.g., at least 5%, 10%, 15%, 20%, 30%, 40%, 50%, 60%, 75%, 80%, 90% or more) as compared to an active agent alone.

Pharmaceutical compositions described herein may be formulated for example including a carrier, such as a pharmaceutical carrier and/or a polymeric carrier, e.g., a liposome or vesicle, and delivered by known methods to a subject in need thereof (e.g., a human or non-human agricultural or domestic animal, e.g., cattle, dog, cat, horse, poultry). Such methods include transfection (e.g., lipid-mediated, cationic polymers, calcium phosphate); electroporation or other methods of membrane disruption (e.g., nucleofection) and viral delivery (e.g., lentivirus, retrovirus, adenovirus, AAV). Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

### Lipid Nanoparticles

Site-specific disrupting agents, expression repressors, or systems as described herein can be delivered using any biological delivery system/formulation including a particle, for example, a nanoparticle delivery system. Nanoparticles include particles with a dimension (e.g. diameter) between about 1 and about 1000 nanometers, between about 1 and about 500 nanometers in size, between about 1 and about 100 nm, between about 30 nm and about 200 nm, between about 50 nm and about 300 nm, between about 75 nm and about 200 nm, between about 100 nm and about 200 nm, and any range therebetween. A nanoparticle has a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In some embodiments, nanoparticles have a greatest dimension ranging between 25 nm and 200 nm. Nanoparticles as described herein comprise delivery systems that may be provided in any form, including but not limited to solid, semisolid, emulsion, or colloidal nanoparticles. A nanoparticle delivery system may include but not limited to lipid-based systems, liposomes, micelles, microvesicles, exosomes, or gene gun. In one embodiment, the nanoparticle is a lipid nanoparticle (LNP). In some embodiments, the LNP is a particle that comprises a plurality of lipid molecules physically associated with each other by intermolecular forces. In some embodiments, an LNP may comprise multiple components, e.g., 3-4 components. In one embodiment, the expression repressor or a pharmaceutical composition comprising said expression repressor (or a nucleic acid encoding the same, or pharmaceutical composition comprising a nucleic acid encoding said expression repressor) is encapsulated in an LNP. In one embodiment, the system or a pharmaceutical composition comprising said system (or a nucleic acid encoding the same, or pharmaceutical composition comprising nucleic acid encoding the said system) is encapsulated in an LNP. In some embodiments, the nucleic acid encoding the first expression repressor and the nucleic acid encoding the second expression repressor are present in same LNP. In some embodiments, the nucleic acid encoding the first expression repressor and the nucleic acid encoding the second expression repressor are present in different LNPs. In some embodiments, the nucleic acid encoding the expression repressor and the nucleic acid encoding the site-specific disrupting agent are present in same LNP. In some embodiments, the nucleic acid encoding the expression repressor and the nucleic acid encoding the site-specific disrupting agent are present in different LNPs. In one embodiment, the site-specific disrupting agent or a pharmaceutical composition comprising said site-specific disrupting agent (or a nucleic acid encoding the same, or pharmaceutical composition comprising a nucleic acid encoding said site specific disrupting agent) is encapsulated in an LNP. In one embodiment, the system or a pharmaceutical composition comprising said system (or a nucleic acid encoding the same, or pharmaceutical composition comprising nucleic acid encoding the said system) is encapsulated in an LNP. In some embodiments, the nucleic acid encoding the first site-specific disrupting agent and the nucleic acid encoding the second site-specific disrupting agent are present in same LNP. In some embodiments, the nucleic acid encoding the first site-specific disrupting agent and the nucleic acid encoding the second site-specific disrupting agent are present in different LNPs. Preparation of LNPs and the modulating agent encapsulation may be used/and or adapted from Rosin et al, Molecular Therapy, vol. 19, no. 12, pages 1286-2200, December 2011). In some embodiments, lipid nanoparticle compositions disclosed herein are useful for expression of protein encoded by mRNA. In some embodiments, nucleic acids, when present in the lipid nanoparticles, are resistant in aqueous solution to degradation with a nuclease.

In some embodiments, the LNP formulations may include a CCD lipid, a neutral lipid, and/or a helper lipid. In some embodiments, the LNP formulation comprises an ionizable lipid. In some embodiments, an ionizable lipid may be a cationic lipid, an ionizable cationic lipid, or an amine-containing lipid that can be readily protonated. In some embodiments, the lipid is a cationic lipid that can exist in a positively charged or neutral form depending on pH. In some embodiments, the cationic lipid is a lipid capable of being positively charged, e.g., under physiological conditions. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol and polymer conjugated lipids.

In some embodiments, LNP formulation (e.g., MC3 and/or SSOP) includes cholesterol, PEG, and/or a helper lipid. The LNPs may be, e.g., microspheres (including uni-lamellar and multi-lamellar vesicles, lamellar phase lipid bilayers that, in some embodiments, are substantially spherical.

In some embodiments, the LNP can comprise an aqueous core, e.g., comprising a nucleic acid encoding a site-specific disrupting agent or a system as disclosed herein. In some embodiments of the present disclosure, the cargo for the LNP formulation includes at least one guide RNA. In some embodiments, the cargo, e.g., a nucleic acid encoding an expression repressor, or a system as disclosed herein, may be adsorbed to the surface of an LNP, e.g., an LNP comprising a cationic lipid. In some embodiments, the cargo, e.g., a nucleic acid encoding an expression repressor, or a system as disclosed herein may be associated with the LNP. In some embodiments, the cargo, e.g., a nucleic acid encoding an expression repressor, or a system as disclosed herein, may be encapsulated, e.g., fully encapsulated and/or partially encapsulated in an LNP. In some embodiments, the cargo, e.g., a nucleic acid encoding a site-specific disrupting agent, or a system as disclosed herein, may be adsorbed to the surface of an LNP, e.g., an LNP comprising a cationic lipid. In some embodiments, the cargo, e.g., a nucleic acid encoding a site-specific disrupting agent, or a system as disclosed herein may be associated with the LNP. In some embodiments, the cargo, e.g., a nucleic acid encoding a site-specific disrupting agent, or a system as disclosed herein, may be encapsulated, e.g., fully encapsulated and/or partially encapsulated in an LNP.

In some embodiments, an LNP comprising a cargo may be administered for systemic delivery, e.g., delivery of a therapeutically effective dose of cargo that can result in a broad exposure of an active agent within an organism. Systemic delivery of lipid nanoparticles can be for example, intravenous, pulmonary, bronchial, intraarterial, subcutaneous, and intraperitoneal delivery. In some embodiments, systemic delivery of lipid nanoparticles is by intravenous delivery. In some embodiments, an LNP comprising a cargo may be administered for local delivery, e.g., delivery of an active agent directly to a target site within an organism. In some embodiments, an LNP may be locally delivered into a disease site, e.g., a tumor, other target site, e.g., a site of inflammation, or to a target organ, e.g., the liver, lung, stomach, colon, pancreas, uterus, breast, lymph nodes, and the like. In some embodiments, an LNP as disclosed herein may be locally delivered to a specific cell, e.g., hepatocytes, stellate cells, Kupffer cells, endothelial, alveolar, and/or epithelial cells. In some embodiments, an LNP as disclosed herein may be locally delivered to a specific tumor site, e.g., subcutaneous, orthotopic.

The LNPs may be formulated as a dispersed phase in an emulsion, micelles, or an internal phase in a suspension. In some embodiments, the LNPs are biodegradable. In some embodiments, the LNPs do not accumulate to cytotoxic levels or cause toxicity in vivo at a therapeutically effective dose. In some embodiments, the LNPs do not accumulate to cytotoxic levels or cause toxicity in vivo after repeat administrations at a therapeutically effective dose. In some embodiments, the LNPs do not cause an innate immune response that leads to a substantially adverse effect at a therapeutically effective dose.

In some embodiments, the LNP used, comprises the formula (6Z,9Z,28Z,31Z)-heptatriacont-6,9,28,31-tetraene-19-yl 4-(dimethylamino) butanoate or ssPalmO-phenyl-P4C2 (ssPalmO-Phe, SS-OP). In some embodiments, the LNP formulation comprises the formula, (6Z,9Z,28Z,31Z)-heptatriacont-6,9,28,31-tetraene-19-yl 4-(dimethylamino)butanoate (MC3), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., MC3 LNP or ssPalmO-phenyl-P4C2 (ssPalmO-Phe, SS-OP), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., SSOP-LNP.

In some embodiments, the LNP used comprises SM-102 (9-Heptadecanyl 8-{(2-hydroxyethyl)[6-oxo-6-(undecyloxy)hexyl]amino}octanoate).

Liposomes are spherical vesicle structures composed of a uni- or multi-lamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

### Uses

The present disclosure is further directed to uses of the expression repressors and/or site-specific disrupting agents, or systems disclosed herein. Among other things, in some embodiments such provided technologies may be used to achieve modulation, e.g., repression, of expression of a target plurality of genes and, for example, enable control of the activity, delivery, and penetrance of one or more products of a target plurality of genes, e.g., in a cell. In some embodiments, a cell is a mammalian, e.g., human, cell. In some embodiments, a cell is a somatic cell. In some embodiments, a cell is a primary cell. For example, in some embodiments, a cell is a mammalian somatic cell. In some embodiments, a mammalian somatic cell is a primary cell. In some embodiments, a mammalian somatic cell is a non-embryonic cell.

In some embodiments, the expression repressors or expression repressor systems disclosed herein can be used to treat cancer in a subject in need thereof. In some embodiments, the cancer is a solid tumor, lung cancer (e.g., non-small cell lung cancer), breast cancer, hepatocellular carcinoma (HCC), prostate cancer, colon cancer, skin cancer, cervical cancer, ovarian cancer, uterine endometrioid carcinoma, endometrial cancer, mature B-cell lymphoma, bladder cancer, esophagogastric cancer, esophageal adenocarcinoma, bone cancer, melanoma, hepatobiliary cancer, thyroid cancer, mature B-cell neoplasms, glioma, head-neck squamous cell carcinoma, kidney renal clear cell carcinoma, pancreatic cancer (e.g., pancreatic ductal adenocarcinoma), sarcoma, or stomach adenocarcinoma.

### Modulating Gene Expression

The present disclosure is further directed, in part, to a method of modulating, e.g., decreasing, expression of a target plurality of genes, comprising providing an expression repressor and/or a site-specific disrupting agent, or a system described herein (or a nucleic acid encoding the same, or pharmaceutical composition comprising said expression repressor and/or site-specific disrupting agent, or nucleic acid encoding the same), and contacting the target plurality of genes, a genomic complex component (e.g., a genomic regulatory element (e.g., a transcription factor), an anchor sequence, or an ASMC) associated with the target plurality of genes (e.g., an E1 cRE and/or a genomic complex (e.g., ASMC) comprising the target plurality of genes) with the expression repressor and/or site-specific disrupting agent, or a system. In some embodiments, modulating, e.g., decreasing, expression of a target plurality of genes comprises modulation of transcription of a gene of the target plurality of genes as compared with a reference value, e.g., transcription of the gene in the absence of the expression repressor and/or site-specific disrupting agent, or a system. In some embodiments, the method of modulating, e.g., decreasing, expression of a target plurality of genes is used ex vivo, e.g., on a cell from a subject, e.g., a mammalian subject, e.g., a human subject. In some embodiments, the cell is a mammalian, e.g., human, cell. In some embodiments, the cell is a somatic cell. In some embodiments, the cell is a primary cell. In some embodiments, the method of modulating, e.g., decreasing, expression of a target plurality of genes is used in vivo, e.g., on a mammalian subject, e.g., a human subject. In some embodiments, the method of modulating, e.g., decreasing, expression of a target plurality of genes is used in vitro, e.g., on a cell or cell line described herein.

Without wishing to be bound by theory, in some embodiments, an expression repressor or a system may modulate the expression of a target plurality of genes by binding to a genomic regulatory element, e.g., a cRE, e.g., an E1 cRE, operably linked the target plurality of genes, and having one, two, or all of the following effects: physically or sterically blocking (e.g., competitively inhibiting) binding of a factor to the cRE; epigenetically modifying the target plurality of genes, or a genomic regulatory element (e.g., transcription control element) operably linked to the target plurality of genes; or genetically modifying the target plurality of genes or a genomic regulatory element (e.g., transcription control element) operably linked to the target plurality of genes.

Without wishing to be bound by theory, in some embodiments it is thought that a site-specific disrupting agent or a system may modulate the expression of a target plurality of genes by binding to an anchor sequence of a genomic complex, e.g., ASMC, comprising the target plurality of genes, and having one, two, or all of the following effects: physically or sterically blocking (e.g., competitively inhibiting) binding of a genomic complex component (e.g., nucleating polypeptide) to the anchor sequence; epigenetically modifying the target plurality of genes, a genomic regulatory element (e.g., transcription control element) operably linked to the target plurality of genes, or an anchor sequence proximal to the target plurality of genes or associated with an anchor sequence-mediated conjunction comprising the target plurality of genes (e.g., thereby decreasing and/or eliminating binding of a genomic complex component, e.g., nucleating polypeptide, to the anchor sequence); or genetically modifying the target plurality of genes, a genomic regulatory element (e.g., transcription control element) operably linked to the target plurality of genes, or an anchor sequence proximal to the target plurality of genes or associated with an anchor sequence-mediated conjunction comprising the target plurality of genes (e.g., thereby decreasing and/or eliminating binding of a genomic complex component, e.g., nucleating polypeptide, to the anchor sequence).

In some embodiments, a method described herein modulates, e.g., decreases, the expression of two or more genes of a target plurality of genes. In some embodiments, a method described herein modulates, e.g., decreases, the expression of at least 2, 3, 4, 5, 6, 7, 8, 9, 10, 11, 12, 13, 14, 15, 16, 17, 18, 19, or 20 (and optionally, no more than 2, 3, 4, 5, 6, 7, 8, 9, 10, 11, 12, 13, 14, 15, 16, 17, 18, 19, 20, or 30) genes of a target plurality of genes. In some embodiments, a method described herein modulates, e.g., decreases, the expression of 2-20, 2-18, 2-16, 2-14, 2-12, 2-10, 2-9, 2-8, 2-7, 2-6, 2-5, 2-4, 2-3, 3-20, 3-18, 3-16, 3-14, 3-12, 3-10, 3-9, 3-8, 3-7, 3-6, 3-5, 3-4, 4-20, 4-18, 4-16, 4-14, 4-12, 4-10, 4-9, 4-8, 4-7, 4- 6, 4-5, 5-20, 5-18, 5-16, 5-14, 5-12, 5-10, 5-9, 5-8, 5-7, 5-6, 6-20, 6-18, 6-16, 6-14, 6-12, 6-10, 6-9, 6-8, 6-7, 7-20, 7-18, 7-16, 7-14, 7-12, 7-10, 7-9, 7-8, 8-20, 8-18, 8-16, 8-14, 8-12, 8-10, 8-9, 9-20, 9-18, 9-16, 9-14, 9-12, 9-10, 10-20, 10-18, 10-16, 10-14, 10-12, 12-20, 12-18, 12-16, 12-14, 14-20, 14-18, 14-16, 16-20, 16-18, or 18-20 genes of a target plurality of genes. In some embodiments, a method described herein modulates, e.g., decreases, the expression of each gene (e.g., all genes) of a target plurality of genes.

In some embodiments, a method described herein modulates, e.g., decreases, the expression of a gene of a target plurality of genes, wherein one or more (e.g., 1, 2, 3, 4, 5, 6, 7, 8, 9, 10 or more, e.g., all) of the genes is a cytokine, an interleukin, a transcription factor (e.g., an interferon regulatory transcription factor), intercellular adhesion molecule (ICAM), or an interferon receptor. In some embodiments, a method described herein modulates, e.g., decreases, the level of RNA, e.g., mRNA, produced from one or more (e.g., 1, 2, 3, 4, 5, 6, 7, 8, 9, 10 or more, e.g., all) genes of the target plurality of genes. In some embodiments, modulating expression comprises decreasing the level of protein produced from one or more (e.g., 1, 2, 3, 4, 5, 6, 7, 8, 9, 10 or more, e.g., all) genes of the target plurality of genes. In some embodiments, modulating expression comprises both decreasing the level of mRNA and protein produced from one or more (e.g., 1, 2, 3, 4, 5, 6, 7, 8, 9, 10 or more, e.g., all) genes of the target plurality of genes. In some embodiments, the decrease is a decrease of at least 10%, 20%, 30%, 40%, 50%, 60%, 70%, 80%, or 90% compared to pre-treatment levels or levels in the absence of the site-specific disrupting agent.

In some embodiments, a method described herein modulates, e.g., decreases, the expression of one or more of (e.g., 1, 2, 3, or all of) human CXCL1, CXCL2, CXCL3, CXCL4, CXCL5, CXCL6, CXCL7, and IL8, e.g., upon stimulation of the cell with TNF-alpha, e.g., using an assay of any of Examples 2 or 4-11. In some embodiments, a method described herein modulates, e.g., decreases, the expression of one or more of (e.g., 1, 2, 3, or all of) mice CXCL1, CXCL2, CXCL3, CXCL4, CXCL5, CXCL7, and CXCL15, e.g., upon stimulation of the cell with TNF-alpha, e.g., using an assay of Example 14. In some embodiments, a method described herein modulates, e.g., decreases, the expression or one or more of (e.g., 1, 2, 3, or all of) human CXCL1, CXCL2, CXCL3, CXCL4, CXCL5, CXCL6, CXCL7, and IL8, e.g., upon stimulation of the cell with IL-1A, e.g., using an assay of any of Examples 1-29.

In some embodiments, a method described herein decreases binding of a factor, e.g., a transcription factor, e.g., P65, to an enhancer sequence. In some embodiments, contacting a cell or administering an expression repressor results in a decrease in binding of a transcription factor, e.g., P65, to an enhancer sequence (e.g., an E1 enhancer sequence operably linked to a target plurality of genes). In some embodiments, contacting a cell or administering an expression repressor results in a complete loss of binding or a decrease of at least 50, 60, 70, 80, 90, 95, or 99%, e.g., as measured by ChIP and/or quantitative PCR, relative to the binding of the transcription factor (e.g., P65) to the enhancer sequence prior to treatment with the expression repressor or the system or in the absence of the expression repressor, or the system.

The present disclosure is further directed, in part, to a method of treating a condition associated with over-expression of a target plurality of genes in a subject, comprising administering to the subject an expression repressor, a system, a nucleic acid, a vector, a cell, or a pharmaceutical composition described herein. Conditions associated with over-expression of particular genes are known to those of skill in the art. Such conditions include, but are not limited to, metabolic disorders, neuromuscular disorders, cancer (e.g., solid tumors, lung cancer (e.g., non-small cell lung cancer), breast cancer, hepatocellular carcinoma (HCC), prostate cancer, colon cancer, skin cancer, cervical cancer, ovarian cancer, uterine endometrioid carcinoma, endometrial cancer, mature B-cell lymphoma, bladder cancer, esophagogastric cancer, esophageal adenocarcinoma, bone cancer, melanoma, hepatobiliary cancer, thyroid cancer, mature B-cell neoplasms, glioma, head-neck squamous cell carcinoma, kidney renal clear cell carcinoma, pancreatic cancer (e.g., pancreatic ductal adenocarcinoma), sarcoma, or stomach adenocarcinoma), fibrosis, diabetes, urea disorders, immune disorders, inflammation, and arthritis. In some embodiments, the disorder is an auto-immune disorder. In some embodiments, the disorder is associated with or caused by an infection, e.g., a viral infection, e.g., SARS-Cov2 viral infection.

In some embodiments, a method described herein decreases binding of a nucleating polypeptide, e.g., CTCF, to an anchor sequence. In some embodiments, contacting a cell or administering a site-specific disrupting agent results in a decrease in binding of a nucleating polypeptide, e.g., CTCF, to an anchor sequence (e.g., an anchor sequence of an ASMC comprising a target plurality of genes). In some embodiments, contacting a cell or administering a site-specific disrupting agent results in a complete loss of binding or a decrease of at least 50, 60, 70, 80, 90, 95, or 99%, e.g., as measured by ChIP and/or quantitative PCR, relative to the binding of the nucleating polypeptide (e.g., CTCF) to the anchor sequence prior to treatment with the site-specific disrupting agent or the system or in the absence of the site-specific disrupting agent or the system.

The present disclosure is further directed, in part, to a method of treating a condition associated with over-expression of a target plurality of genes in a subject, comprising administering to the subject a site-specific disrupting agent, a system, a nucleic acid, a vector, a cell, or a pharmaceutical composition described herein. Conditions associated with over-expression of particular genes are known to those of skill in the art. Such conditions include, but are not limited to, metabolic disorders, neuromuscular disorders, cancer (e.g., solid tumors, lung cancer (e.g., non-small cell lung cancer), breast cancer, hepatocellular carcinoma (HCC), prostate cancer, colon cancer, skin cancer, cervical cancer, ovarian cancer, uterine endometrioid carcinoma, endometrial cancer, mature B-cell lymphoma, bladder cancer, esophagogastric cancer, esophageal adenocarcinoma, bone cancer, melanoma, hepatobiliary cancer, thyroid cancer, mature B-cell neoplasms, glioma, head-neck squamous cell carcinoma, kidney renal clear cell carcinoma, pancreatic cancer (e.g., pancreatic ductal adenocarcinoma), sarcoma, or stomach adenocarcinoma), fibrosis, diabetes, urea disorders, immune disorders, inflammation, and arthritis. In some embodiments, the disorder is an auto-immune disorder. In some embodiments, the disorder is associated with or caused by an infection, e.g., a viral infection, e.g., SARS-Cov2 viral infection.

The present disclosure is further directed, in part, to a method of modulating, e.g., decreasing, expression of a target plurality of genes, in a cell in a subject, e.g., a human subject. In some embodiments, the subject has a disease or condition. In some embodiments, the disease is an inflammatory disease, e.g., an immune mediated inflammatory disease. In some embodiments, the disease or condition is one or more of rheumatoid arthritis, gout, asthma, neutrophilic asthma, neutrophilic dermatosis, paw edema, acute respiratory disease syndrome (ARDS), COVID-19, psoriasis, inflammatory bowel disease, infection (e.g., by a pathogen, e.g., a bacteria, a viruses, or a fungus), external injury (e.g., scrapes or foreign objects), effects of radiation or chemical injury, osteoarthritis, osteoarthritic joint pain, joint pain, inflammatory pain, acute pain, chronic pain, cystitis, bronchitis, dermatitis, cardiovascular disease, neurodegenerative disease, liver disease, lung disease, kidney disease, pain, swelling, stiffness, tenderness, redness, warmth, or elevated biomarkers related to disease states (e.g., cytokines, immune receptors, or inflammatory markers). In some embodiments, the inflammatory disorder is associated with an infection, e.g., by a virus, e.g., Sars-Cov-2 virus. In some embodiments, the inflammatory disorder is an autoimmune disorder.

Methods and compositions as provided herein may treat a condition associated with over-expression of a target plurality of genes by stably or transiently altering (e.g., decreasing) transcription of a target plurality of genes. In some embodiments, such a modulation persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, such a modulation persists for no more than 10, 9, 8, 7, 6, 5, 4, 3, 2, or 1 years.

In some embodiments, a method or composition provided herein may decrease expression of a gene of a target plurality of genes in a cell by at least 10, 20, 30, 40, 50, 60, 70, 80, 90, or 100% (and optionally up to 100%) relative to expression of the gene of the target plurality of genes in a cell not contacted by the composition or treated with the method. In some embodiments, a method or composition provided herein may decrease expression of each gene of a target plurality of genes in a cell by at least 10, 20, 30, 40, 50, 60, 70, 80, 90, or 100% (and optionally up to 100%) relative to expression of each gene of the target plurality of genes in a cell not contacted by the composition or treated with the method.

In some embodiments, a method provided herein may decrease expression of a target plurality of genes by disrupting the binding of a factor (e.g., a transcription factor) to a cRE operably linked to said target plurality of genes. In some embodiments, contacting a cell or administering an expression repressor results in a decrease in the level of a binding of a factor to a cRE operably linked the target plurality of genes relative to the level of the complex prior to treatment with the expression repressor or the system or in the absence of the expression repressor or the system. In some embodiments, contacting a cell or administering an expression repressor results in a complete loss of the binding of a factor to a cRE, or a decrease of at least 25, 30, 35, 40, 45, 50, 60, 70, 80, 90, 95, or 99%, e.g., as measured by ChIA-PET, ELISA (e.g., to assess gene expression changes), CUT&RUN, ATAC-SEQ, ChIP and/or quantitative PCR, relative to the level of the complex prior to treatment with the expression repressor or the system or in the absence of the expression repressor or the system.

In some embodiments, a method provided herein may modulate, e.g., decrease, expression of a target plurality of genes by disrupting a genomic complex, e.g., an anchor sequence-mediated conjunction, comprising said target plurality of genes. In some embodiments, a method described herein disrupts a genomic complex (e.g., ASMC). In some embodiments, contacting a cell or administering a site-specific disrupting agent results in a decrease in the level of a genomic complex (e.g., ASMC) comprising the target plurality of genes relative to the level of the complex prior to treatment with the site-specific disrupting agent or the system or in the absence of the site-specific disrupting agent or the system. In some embodiments, contacting a cell or administering a site-specific disrupting agent results in a complete loss of the genomic complex, e.g., ASMC, or a decrease of at least 25, 30, 35, 40, 45, 50, 60, 70, 80, 90, 95, or 99%, e.g., as measured by ChIA-PET, ELISA (e.g., to assess gene expression changes), CUT&RUN, ATAC-SEQ, ChIP and/or quantitative PCR, relative to the level of the complex prior to treatment with the site-specific disrupting agent or the system or in the absence of the site-specific disrupting agent or the system.

In some embodiments, methods and compositions as provided herein may treat a condition associated with cascade of inflammation or cytokine storm by decreasing recruitment of cytokines in the site of inflammation. In some embodiments, the cascade of inflammation and/or cytokine storm is associated with an inflammatory disorder, e.g., a viral mediated inflammatory disorder, e.g., COVID-19 infection. In some embodiments, the inflammatory disorder is associated with an infection, e.g., by a virus, e.g., Sars-Cov-2 virus. In some embodiments, the inflammatory disorder is an autoimmune disorder. In some embodiments, the inflammatory disorder is associated with hypoxia. In some embodiments, the inflammatory disorder is associated with ARDS, hypoxia, and/or sepsis. In some embodiments, the infection is a super infection, e.g., caused by more than one pathogen, e.g., a first virus or a bacterium, or a fungus, and a second virus, or a second bacterium, or a second fungus.

### Epigenetic Modification

The present disclosure is further directed, in part, to a method of epigenetically modifying: one or more (e.g., all) genes of a target plurality of genes; a genomic regulatory element (e.g., transcription control element) operably linked to the target plurality of genes or a site proximal to the genomic regulatory element (e.g., transcription control element); an anchor sequence proximal to the target plurality of genes or associated with an anchor sequence-mediated conjunction comprising the target plurality of genes; or a site proximal to said anchor sequence, the method comprising providing an expression repressor and/or site-specific disrupting agent, a system, a nucleic acid encoding the expression repressor and/or site-specific disrupting agent, nucleic acids encoding the components of the system, or pharmaceutical composition comprising said expression repressor and/or site-specific disrupting agent, system or nucleic acid; and contacting the one or more (e.g., all) genes of the target plurality of genes, a genomic regulatory element (e.g., transcription control element) operably linked to the target plurality of genes or a site proximal to the genomic regulatory element (e.g., transcription control element), an anchor sequence proximal to the target plurality of genes or associated with an anchor sequence-mediated conjunction comprising the target plurality of genes, or a site proximal to said anchor sequence with the expression repressor and/or site-specific disrupting agent, or the system, thereby epigenetically modifying the target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence.

In some embodiments, a method of epigenetically a target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence comprises increasing or decreasing DNA methylation of the target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence. In some embodiments, a method of epigenetically modifying target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence comprises increasing or decreasing histone methylation of a histone associated with the target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence. In some embodiments, a method of epigenetically modifying a target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence comprises decreasing histone acetylation of a histone associated with the target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence. In some embodiments, a method of epigenetically modifying a target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence comprises increasing or decreasing histone sumoylation of a histone associated with the target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence. In some embodiments, a method of epigenetically modifying a target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence comprises increasing or decreasing histone phosphorylation of a histone associated with the target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence.

In some embodiments, a method of epigenetically modifying a target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence may decrease the level of the epigenetic modification by at least 10, 20, 30, 40, 50, 60, 70, 80, 90, or 100% (and optionally up to 100%) relative to the level of the epigenetic modification at that site in a cell not contacted by the composition or treated with the method. In some embodiments, a method of epigenetically modifying a target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence may increase the level of the epigenetic modification by at least 10, 20, 30, 40, 50, 60, 70, 80, 90, 100, 120, 140, 160, 180, 200, 300, 400, 500, 600, 700, 800, 900, or 100000 (and optionally up to 200, 300, 400, 500, 600, 700, 800, 900, 1000, or 2000%) relative to the level of the epigenetic modification at that site in a cell not contacted by the composition or treated with the method. In some embodiments epigenetic modification of a target plurality of genes, a genomic regulatory element (e.g., transcription control element) operably linked to the target plurality of genes or site proximal to the genomic regulatory element (e.g., transcription control element), an anchor sequence proximal to the target plurality of genes or associated with an anchor sequence-mediated conjunction comprising the target plurality of genes, or a site proximal to said anchor sequence may modify the level of expression of the target plurality of genes, e.g., as described herein.

In some embodiments, an epigenetic modification produced by a method described herein persists for at least about 1 hour to about 30 days, or at least about 2 hours, 6 hours, 12 hours, 18 hours, 24 hours, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time there between. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, such a modulation persists for no more than 10, 9, 8, 7, 6, 5, 4, 3, 2, or 1 years.

In some embodiments, an expression repressor or a system for use in a method of epigenetically modifying a target plurality of genes, a genomic regulatory element (e.g., transcription control element) operably linked to the target plurality of genes or a site proximal to the genomic regulatory element (e.g., transcription control element), an anchor sequence proximal to the target plurality of genes or associated with an anchor sequence-mediated conjunction comprising the target plurality of genes, or a site proximal to said anchor sequence comprises an effector moiety that comprises an epigenetic modifying moiety. For example, an effector moiety may comprise an epigenetic modifying moiety with DNA methyltransferase activity, and an endogenous or naturally occurring target sequence (e.g., a gene of a target plurality of genes, a genomic regulatory element (e.g., transcription control element) operably linked to the target plurality of genes or a site proximal to the genomic regulatory element (e.g., transcription control element), an anchor sequence proximal to the target plurality of genes or associated with an anchor sequence-mediated conjunction comprising the target plurality of genes, or a site proximal to said anchor sequence) may be altered to increase its methylation (e.g., decreasing interaction of a transcription factor with a portion of a gene of a target plurality of genes or genomic regulatory element (e.g., transcription control element), decreasing binding of a nucleating protein to an anchor sequence, and/or disrupting or preventing an anchor sequence-mediated conjunction), or may be altered to decrease its methylation (e.g., increasing interaction of a transcription factor with a portion of a gene of a target plurality of genes or genomic regulatory element (e.g., transcription control element), increasing binding of a nucleating protein to an anchor sequence, and/or promoting or increasing strength of an anchor sequence-mediated conjunction).

In some embodiments, a site-specific disrupting agent or a system for use in a method of epigenetically modifying a target plurality of genes, a genomic regulatory element (e.g., transcription control element) operably linked to the target plurality of genes, an anchor sequence proximal to the target plurality of genes or associated with an anchor sequence-mediated conjunction comprising the target plurality of genes, or a site proximal to said anchor sequence comprises an effector moiety that comprises an epigenetic modifying moiety. For example, an effector moiety may comprise an epigenetic modifying moiety with DNA methyltransferase activity, and an endogenous or naturally occurring target sequence (e.g., a gene of a target plurality of genes, a genomic regulatory element (e.g., transcription control element) operably linked to the target plurality of genes, an anchor sequence proximal to the target plurality of genes or associated with an anchor sequence-mediated conjunction comprising the target plurality of genes, or a site proximal to said anchor sequence) may be altered to increase its methylation (e.g., decreasing interaction of a transcription factor with a portion of a gene of a target plurality of genes or genomic regulatory element (e.g., transcription control element), decreasing binding of a nucleating protein to an anchor sequence, and/or disrupting or preventing an anchor sequence-mediated conjunction), or may be altered to decrease its methylation (e.g., increasing interaction of a transcription factor with a portion of a gene of a target plurality of genes or genomic regulatory element (e.g., transcription control element), increasing binding of a nucleating protein to an anchor sequence, and/or promoting or increasing strength of an anchor sequence-mediated conjunction).

### Genetic Modification

The present disclosure is further directed, in part, to a method of genetically modifying one or more (e.g., one, two, three, or all) genes of a target plurality of genes, a genomic regulatory element (e.g., transcription control element) operably linked to the target plurality of genes or a site proximal to the genomic regulatory element (e.g., transcription control element), or an anchor sequence proximal to the target plurality of genes or associated with an anchor sequence-mediated conjunction comprising the target plurality of genes, the method comprising providing an expression repressor and/or a site-specific disrupting agent, or a system or nucleic acid encoding the same or pharmaceutical composition comprising said expression repressor and/or said site-specific disrupting agent, system, or nucleic acid; and contacting the one or more (e.g., one, two, three, or all) genes of the target plurality of genes, a genomic regulatory element (e.g., transcription control element) operably linked to the target plurality of genes, or an anchor sequence proximal to the target plurality of genes or associated with an anchor sequence-mediated conjunction comprising the target plurality of genes with the expression repressor and/or site-specific disrupting agent, thereby genetically modifying the target plurality of genes, a genomic regulatory element (e.g., transcription control element) operably linked to the target plurality of genes, or an anchor sequence proximal to the target plurality of genes or associated with an anchor sequence-mediated conjunction comprising the target plurality of genes.

Genetic modification may comprise introducing one or more of an insertion, deletion, or substitution into a gene of a target plurality of genes, a genomic regulatory element (e.g., transcription control element) operably linked to the target plurality of genes, or an anchor sequence proximal to the target plurality of genes or associated with an anchor sequence-mediated conjunction comprising the target plurality of genes. In some embodiments, an insertion comprises addition of at least 150, 200, 250, 300, 400, 500, 600, 700, 800, 900, 1000, 1200, 1400, 1600, 1800, or 2000 nucleotides (and optionally no more than 3000, 2500, 2000, 1500, 1000, 900, 800, 700, 600, 500, 400, 300, or 200 nucleotides). In some embodiments, an insertion comprises addition of at least 1, 2, 3, 4, 5, 6, 7, 8, 9, 10, 15, 20, 25, 30, 35, 40, 50, 60, 70, 80, 90, or 100 nucleotides (and optionally no more than 200, 150, 100, 90, 80, 70, 60, 50, 40, 35, 30, 25, 20, 15, 10, 9, 8, 7, 6, 5, 4, 3, 2, or 1 nucleotide). In some embodiments, the insertion comprises addition of 1-10, 1-9, 1-8, 1-7, 1-6, 1-5, 1-4, 1-3, or 1-2 nucleotides. In some embodiments, a deletion comprises removal of at least 150, 200, 250, 300, 400, 500, 600, 700, 800, 900, 1000, 1200, 1400, 1600, 1800, or 2000 nucleotides (and optionally no more than 3000, 2500, 2000, 1500, 1000, 900, 800, 700, 600, 500, 400, 300, or 200 nucleotides). In some embodiments, a deletion comprises removal of at least 1, 2, 3, 4, 5, 6, 7, 8, 9, 10, 15, 20, 25, 30, 35, 40, 50, 60, 70, 80, 90, or 100 nucleotides (and optionally no more than 200, 150, 100, 90, 80, 70, 60, 50, 40, 35, 30, 25, 20, 15, 10, 9, 8, 7, 6, 5, 4, 3, 2, or 1 nucleotide). In some embodiments, the deletion comprises removal of 1-10, 1-9, 1-8, 1-7, 1-6, 1-5, 1-4, 1-3, or 1-2 nucleotides. In some embodiments, a substitution comprises alteration of at least 1, 2, 3, 4, 5, 6, 7, 8, 9, 10, 15, 20, 25, 30, 35, 40, 50, 60, 70, 80, 90, or 100 nucleotides (and optionally no more than 200, 150, 100, 90, 80, 70, 60, 50, 40, 35, 30, 25, 20, 15, 10, 9, 8, 7, 6, 5, 4, 3, 2, or 1 nucleotide). In some embodiments, the substitution comprises alteration of 1-10, 1-9, 1-8, 1-7, 1-6, 1-5, 1-4, 1-3, or 1-2 nucleotides.

In some embodiments, a genetic modification comprises an insertion, deletion, or substitution to a genomic regulatory element (e.g., a cRE) operably linked to the target plurality of genes. In some embodiments, the genetic modification alters (e.g., decreases or increases) the binding of a factor to the cRE (e.g., an enhancer, e.g., an E1 cRE). In some embodiments, the genetic modification abrogates (e.g., via an insertion, deletion, or substitution), wholly or in part, an enhancer sequence, thereby decreasing or abolishing the binding of a factor to the enhancer sequence, e.g., and decreasing the presence of or abolishing a genomic regulatory element comprising said enhancer sequence. Without wishing to be bound by theory, the disclosure contemplates use of an expression repressor with genetic modification functionality to introduce an insertion, deletion, or substitution into an enhancer sequence that is operably linked to a target plurality of genes.

In some embodiments, the genetic modification comprises insertion of a sequence comprising an enhancer sequence. Without wishing to be bound by theory, the disclosure contemplates use of an expression repressor or a system with genetic modification functionality to introduce an exogenous enhancer sequence into a genomic regulatory element (e.g., transcription control element) operably linked to the target plurality of genes.

In some embodiments, a genetic modification comprises an insertion, deletion, or substitution to an anchor sequence, e.g., associated with an ASMC comprising the target plurality of genes. In some embodiments, the genetic modification alters (e.g., decreases or increases) the binding of a genomic complex component, e.g., a nucleating polypeptide, to the anchor sequence. In some embodiments, the genetic modification abrogates (e.g., via an insertion, deletion, or substitution), wholly or in part, an anchor sequence, thereby decreasing or abolishing the binding of a nucleating polypeptide to the anchor sequence, e.g., and decreasing the presence of or abolishing an ASMC comprising said anchor sequence. Without wishing to be bound by theory, the disclosure contemplates use of a site-specific disrupting agent with genetic modification functionality to introduce an insertion, deletion, or substitution into an anchor sequence to decrease or eliminate the anchor sequence's participation in a genomic complex, e.g., ASMC, that comprises a target plurality of genes. As described elsewhere herein, such an alteration is expected to disrupt the genomic complex, e.g., ASMC, and may decrease expression of the target plurality of genes.

In some embodiments, the genetic modification comprises insertion of a sequence comprising an anchor sequence. Without wishing to be bound by theory, the disclosure contemplates use of a site-specific disrupting agent or a system with genetic modification functionality to introduce an exogenous anchor sequence into a gene of a target plurality of genes, a genomic regulatory element (e.g., transcription control element) operably linked to the target plurality of genes, or an anchor sequence proximal to the target plurality of genes or associated with an anchor sequence-mediated conjunction comprising the target plurality of genes. It is thought that the presence of a new anchor sequence may disrupt the formation and/or maintenance of a genomic complex, e.g., ASMC, comprising the target plurality of genes, thereby modulating, e.g., decreasing, expression of the target plurality of genes.

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### EXAMPLES

### Example 1: Decreasing Expression of an Exemplary Plurality of Genes

This example describes, in part, experiments demonstrating decreasing expression of a target plurality of genes comprising CXCL1, CXCL2, CXCL3, and IL8 using a site-specific disrupting agent comprising a CRISPR/Cas molecule and guide RNAs comprising the given guide sequences.

Transfection of comprising a site-specific disrupting agent comprising mRNA encoding CRISPR/Cas molecule (Cas9) and sgRNA Cas9/guide RNP complex was carried out by electroporation into THP-1 cells. Cells were cultured in RPMI+10% FBS. A parental line was also analyzed for comparison. 350k cells were plated in quadruplicate for each edited cell line and the parental control into 24 well plates. One hour later 10 ng/ml of TNF alpha (Sigma Cat #654205) was added to 2 wells for each cell line. The remaining 2 wells are untreated as a control.

The edited and parental cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL1, CXCL2, CXCL3 & IL8 specific TaqMan primer/probe sets with the TaqMan® Fast Advanced Master Mix (Thermo Fisher Scientific). CXCL1-3 & IL7 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells.

The CTCF anchors at both boundaries of the Insulated Genomic Domain (IGD) were located using ChIP-seq data, and the CTCF anchor sequences were identified computationally using the known CTCF position weight matrix (JASPAR). CRISPR (Sp Cas9) guides were chosen to target the CTCF anchor sequence.

The guides sequences are listed in the table below.

TABLE 4

| Guide Sequence | Genomic Coordinates | Guide |
|---|---|---|
| GGGGCCACTAGGGACAGGAT (SEQ | chr19:55627120-55627139 | GD-28662 |
| ID NO: 20) (non-target control) | | |
| AGCCCCACCTTGTGGTCAGA (SEQ ID | chr4:74595464-74595486 | GD-28481 |
| NO: 21) | | |
| AGTGCTGCCTTCTGACCACA (SEQ ID | chr4:74595457-74595479 | GD-28482 |
| NO: 22) | | |
| GCTGCCTTCTGACCACAAGG (SEQ ID | chr4:74595460-74595482 | GD-28483 |
| NO: 23) | | |
| CCAGTATAAGCCCCACCTTG (SEQ ID | chr4:74595472-74595494 | GD-28484 |
| NO: 24) | | |
| CTGCCTGTCCCATAAGGAGG (SEQ ID | chr4:75000088-75000110 | GD-28485 |
| NO: 25) | | |
| GCACTGCCTGTCCCATAAGG (SEQ ID | chr4:75000091-75000113 | GD-28486 |
| NO: 26) | | |
| GGTCCTCCTCCTTATGGGAC (SEQ ID | chr4:75000085-75000107 | GD-28487 |
| NO: 27) | | |
| GCCTTGTTTTCGGCTCTAGA (SEQ ID | chr4:75000157-75000179 | GD-28488 |
| NO: 28) | | |
| GCCATCTAGAGCCGAAAACA (SEQ | chr4:75000156-75000178 | GD-28489 |
| ID NO: 29) | | |

### Example 2: Cytokine Expression Decrease in THP-1 Cells at 72 Hours

This example describes, in part, experiments demonstrating decreasing expression of a target plurality of genes comprising CXCL1, CXCL2, CXCL3, and IL8 using a site-specific disrupting agent comprising a CRISPR/Cas molecule and guide RNAs targeted to the anchor sequences of the ASMC comprising the target plurality of genes.

sgRNA and mRNA encoding Cas9 RNPs were electroporated into THP-1 cells. sgRNA sequences (from Example 1) were chosen to target one of the CTCF sites of the ASMC comprising CXCL1, CXCL2, CXCL3, and IL8. The transfected cells were incubated with 10 ng/ml TNF alpha for 24 hrs. After that time, DNA and RNA were isolated using the AllPrep DNA/RNA Kit (Qiagen), following the manufacturer's protocol. RNA samples were retrotranscribed to cDNA using LunaScript® RT SuperMix Kit (New England Biolabs) and analyzed by quantitative PCR using individual CXCL1, CXCL2, CXCL3 & IL8 specific TaqMan primer/probe sets with the TaqMan® Fast Advanced Master Mix (Thermo Fisher Scientific).

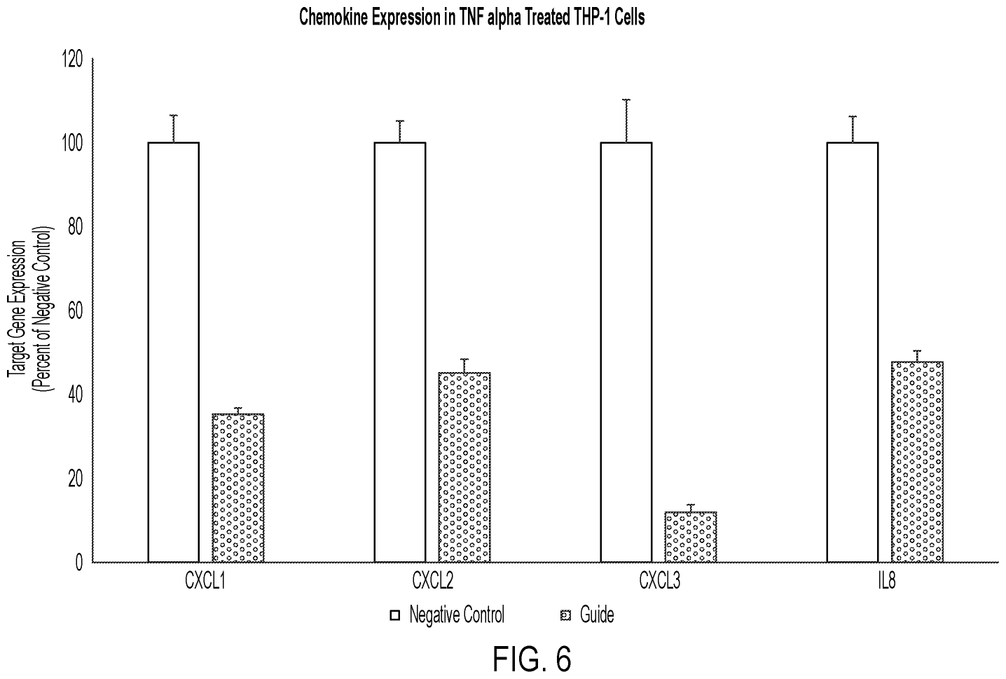

CXCL1-3 & IL8 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results in FIG. 6 show that a site-specific disrupting agent comprising a CRISPR/Cas molecule and an sgRNA can be used to decrease CXCL1, CXCL2, CXCL3, and IL8 expression in THP-1 cells, and that expression is decreased at 72 hours post-treatment. The results also show that LNP delivery of said site-specific disrupting agent can be used to deliver effective amounts of the agent to the target cells.

Similar results were seen when the experiment was repeated with sgRNAs targeting the other CTCF site of the ASMC comprising CXCL1, CXCL2, CXCL3, and IL8 (data not shown).

### Example 3: Cytokine Protein Secretion of THP-1 Cells Decreased by Site-Specific Modulating Agent

This example describes, in part, experiments demonstrating decreasing secretion of CXCL1 and IL-8, two genes of a target plurality of genes, by treating cells using a site-specific disrupting agent comprising a CRISPR/Cas molecule and guide RNAs targeted to the anchor sequences of the ASMC comprising the target plurality of genes.

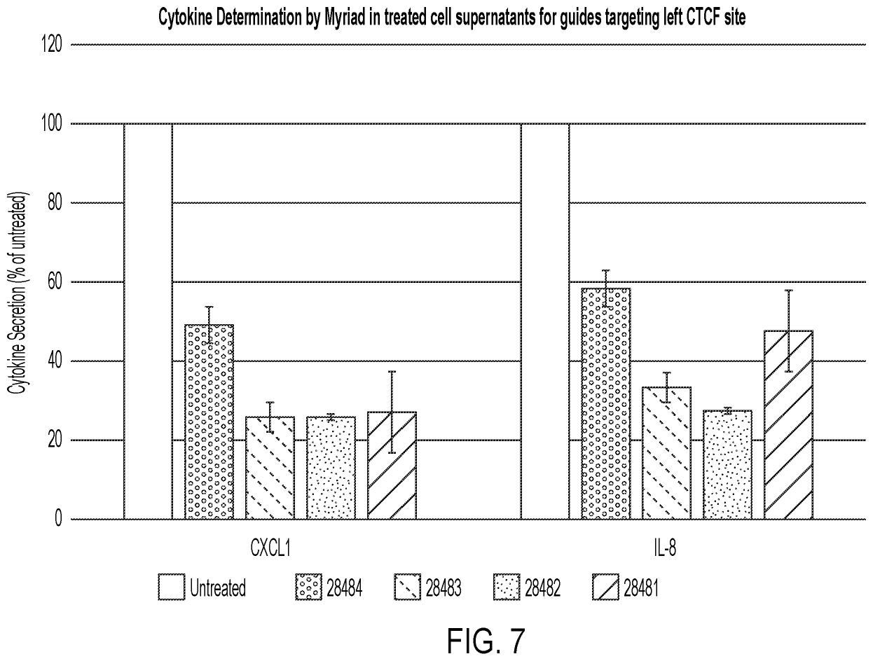

THP-1 cells were electroporated with RNPs comprising sgRNAs and mRNA encoding a site-specific disrupting agent comprising an exemplary CRISPR/Cas molecule (Cas9) as in previous Examples. sgRNAs (from Example 1) were targeted to one of the CTCF sites of the ASMC comprising CXCL1 and IL-8. Cells were stimulated with 10 ng/ml TNF alpha for 24 hours. After that time, cell supernatants were collected and frozen at −80 degrees ° C. Supernatants from cells contacted with 4 different sgRNAs, in addition to the mRNA encoding the CRISPR/Cas molecule, as well as an un-transfected positive control were screened for CXCL1 and IL-8 protein levels on a cytokine panel by Myriad Genetics Inc. FIG. 7 shows diminished levels of CXCL1 and IL8 were seen for each supernatant obtained from cells treated with sgRNA and CRISPR/Cas molecule RNPs, demonstrating a phenotypic response to ASMC disruption (e.g., by disrupting anchor sequence and nucleating polypeptide interactions, e.g., disrupting CTCF binding). This data is in agreement with the decreased mRNA expression seen by qPCR in Example 2.

Similar results were seen when the experiment was repeated with sgRNAs targeting the other CTCF site of the ASMC comprising CXCL1 and IL8 (data not shown).

### Example 4: CXCL3 Expression Decrease as Measured by qPCR

This example describes, in part, experiments demonstrating decreasing expression of CXCL3 by treating THP-1 cells using a site-specific disrupting agent comprising a CRISPR/Cas molecule and a variety of guide RNAs targeted to the anchor sequences of the ASMC comprising the target plurality of genes.

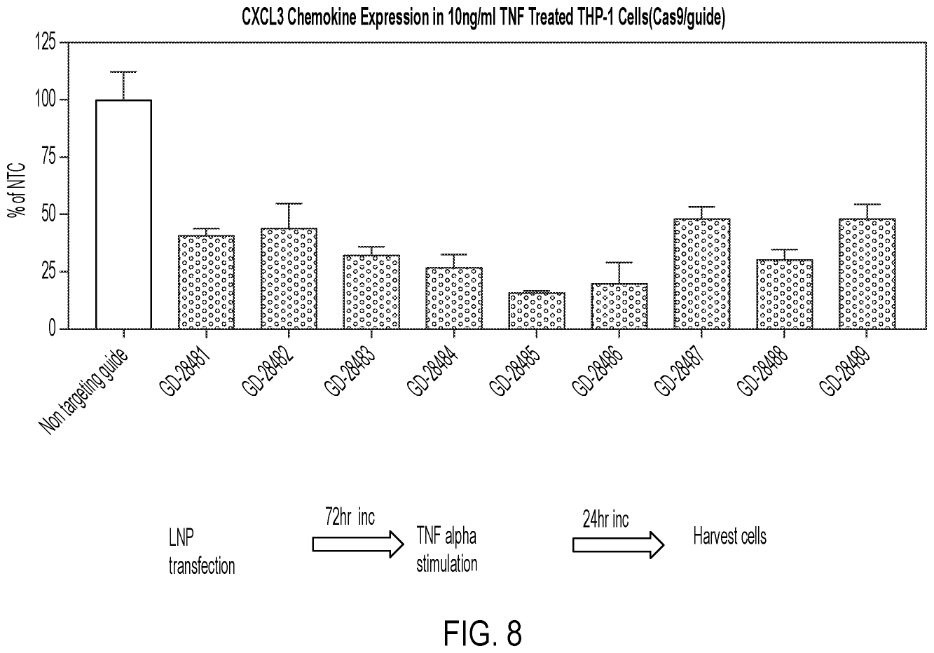

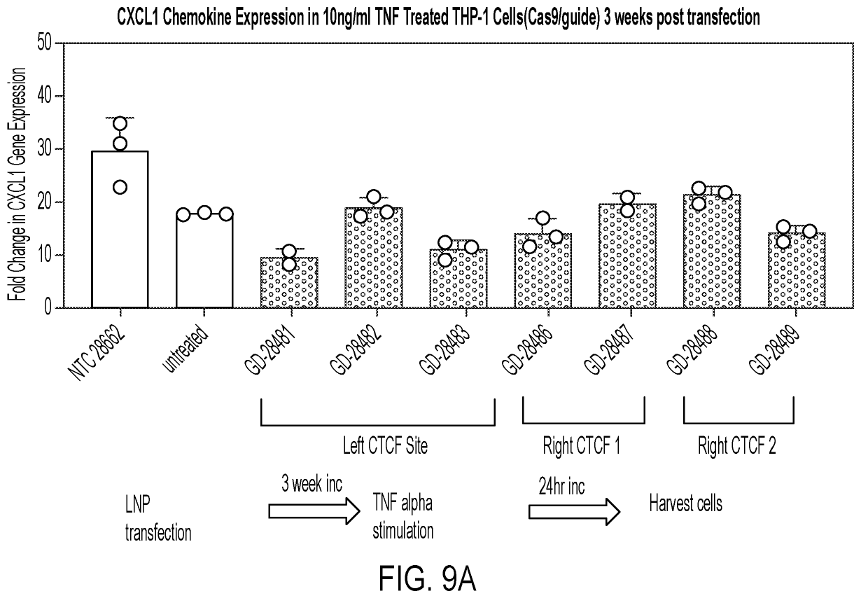

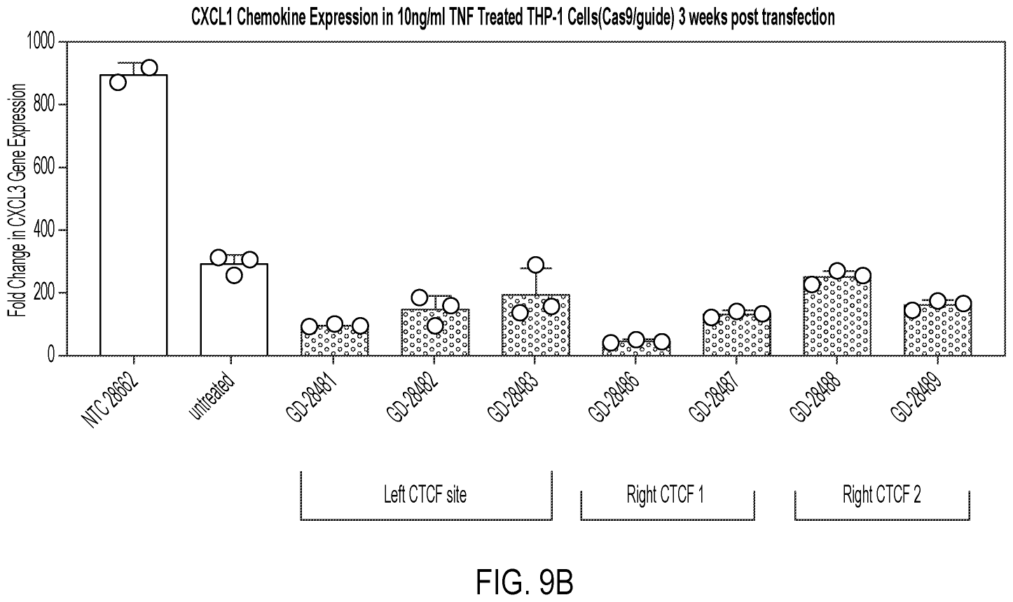

THP-1 cells were grown in RPMI+10% FBS. Cells were transfected with mRNA encoding a CRISPR/Cas molecule (Cas9) and sgRNA targeted to either of the CTCF sites of the ASMC comprising the target plurality of genes using LNPs. sgRNAs (from Example 1) used target the left or right CTCF site as indicated in FIG. 9A. Lipid nanoparticle (LNP) formulation using SSOP lipid mixture was carried out by using the NanoAssemblr® Spark™ from Precision NanoSystems Inc. For experimental conditions, 350k cells/well were plated 72 hrs after LNP transfection for each transfected condition and the parental control into 24 well plates (see FIG. 8 flow chart). One hour later 10 ng/ml of TNF alpha (Sigma Cat #654205) was added to each well. Untreated parental cells were plated with and without 10 ng/ml TNF alpha.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL3 TaqMan primer/probe sets with the TaqMan® Fast Advanced Master Mix (Thermo Fisher Scientific).

CXCL3 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 8 graph) that a site-specific disrupting agent comprising a CRISPR/Cas molecule and several different sgRNAs can be used to decrease CXCL3 expression in THP-1 cells. The results also show that LNP delivery of said site-specific disrupting agent can be used to deliver effective amounts of the agent to the target cells. The results also demonstrate that targeting the anchor sequence on either side of the ASMC comprising the target plurality of genes can decrease expression of the target plurality of genes.

Similar results were seen when the experiment was repeated with sgRNAs targeting the other CTCF site of the ASMC comprising CXCL1, CXCL2, CXCL3, and IL8 (data not shown).

### Example 5: CXCL1 and CXCL3 Expression is Decreased 3 Weeks Post-Transfection

This example describes, in part, experiments demonstrating a stable decrease in expression of CXCL1 and CXCL3 in THP-1 cells three weeks post-transfection with a site-specific disrupting agent comprising a CRISPR/Cas molecule and a variety of guide RNAs targeted to the anchor sequences of the ASMC comprising the target plurality of genes.

Cells and LNP were prepared, and samples analyzed as in Example 4, except that transfected cells were incubated for 3 weeks before TNF alpha stimulation (see FIG. 9A flow chart).

The results show (FIGS. 9A and 9B) that a site-specific disrupting agent comprising a CRISPR/Cas molecule and several different sgRNAs can be used to stably decrease CXCL1 and CXCL3 expression in THP-1 cells up to and including 3 weeks after treatment with LNPs comprising the agent(s). The results also demonstrate that targeting the anchor sequence on either side of the ASMC comprising the target plurality of genes can stably decrease expression of the target plurality of genes.

### Example 6: Agents Comprising KRAB Effector Moieties Decrease CXCL1 Expression

This example describes, in part, experiments demonstrating a decrease in expression of CXCL1 in THP-1 cells after transfection with a site-specific disrupting agent comprising a CRISPR/Cas molecule fused to a transcriptional repressor and a variety of guide RNAs targeted to the anchor sequences of the ASMC comprising the target plurality of genes.

THP-1 cells were grown in RPMI+10% FBS. Cells were transfected with mRNA encoding a CRISPR/Cas molecule fused to a transcriptional repressor, dCas9-KRAB, and sgRNA (from Example 1) targeted to either CTCF site of the ASMC comprising the target plurality of genes using LNPs. Lipid nanoparticle (LNP) formulation using SSOP lipid mixture was carried out by using the NanoAssemblr® Spark™ from Precision NanoSystems Inc. For experimental conditions, 350k cells/well were plated 72 hrs after LNP transfection for each transfected condition and the parental control into 24 well plates. One hour later 10 ng/ml of TNF alpha (Sigma Cat #654205) was added to each well. Untreated parental cells were plated with and without 10 ng/ml TNF alpha. Transfection with mRNA encoding a CRISPR/Cas molecule (Cas9) and the sgRNAs (per the Examples 2, 4, and 5) was performed as a positive control.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL1 specific TaqMan primer/probe sets with the TaqMan® Fast Advanced Master Mix (Thermo Fisher Scientific).

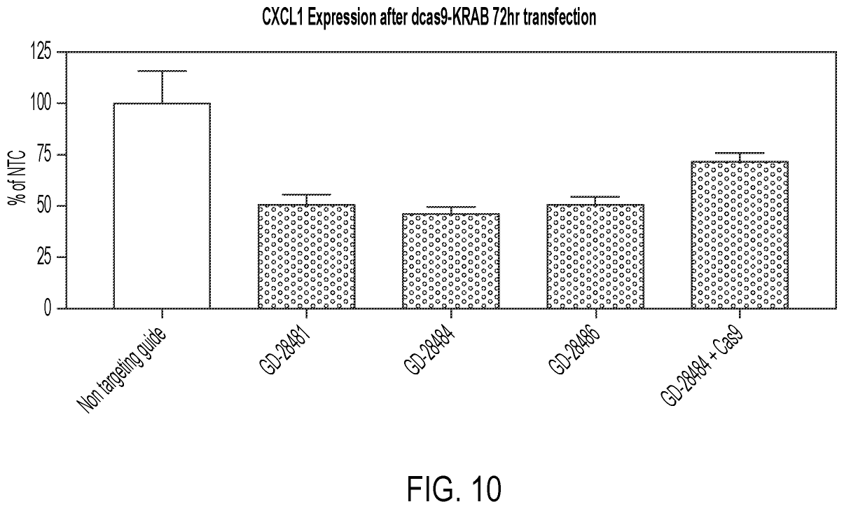

CXCL1 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 10) that a site-specific disrupting agent comprising a catalytically inactive CRISPR/Cas molecule fused to a transcriptional repressor, KRAB, and targeted to CTCF sites by different sgRNAs can be used to decrease CXCL1 expression in THP-1 cells. The decrease in expression was seen 72 hours post-transfection.

### Example 7: Agents Comprising EZH2 Effector Moieties Decrease CXCL1 Expression

This example describes, in part, experiments demonstrating a decrease in expression of CXCL1 in THP-1 cells after transfection with a site-specific disrupting agent comprising a CRISPR/Cas molecule fused to a histone methyltransferase (EZH2) and a variety of guide RNAs targeted to the anchor sequences of the ASMC comprising the target plurality of genes.

THP-1 cells were grown in RPMI+10% FBS. Cells were transfected with mRNA encoding a catalytically inactive CRISPR/Cas molecule (dCas9) fused to a histone deacetylase, dCas9-EZH2, and sgRNA (from Example 1) targeted to either CTCF site of the ASMC comprising the target plurality of genes using LNPs. Lipid nanoparticle (LNP) formulation using SSOP lipid mixture was carried out by using the NanoAssemblr© Spark™ from Precision NanoSystems Inc. For experimental conditions, 350k cells/well were plated 72 hrs after LNP transfection for each transfected condition and the parental control into 24 well plates. One hour later 10 ng/ml of TNF alpha (Sigma Cat #654205) was added to each well. Untreated parental cells were plated with and without 10 ng/ml TNF alpha.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript© RT SuperMix Kit (New England Biolabs) and analyzed by quantitative PCR using individual CXCL1 specific TaqMan primer/probe sets with the TaqMan® Fast Advanced Master Mix (Thermo Fisher Scientific).

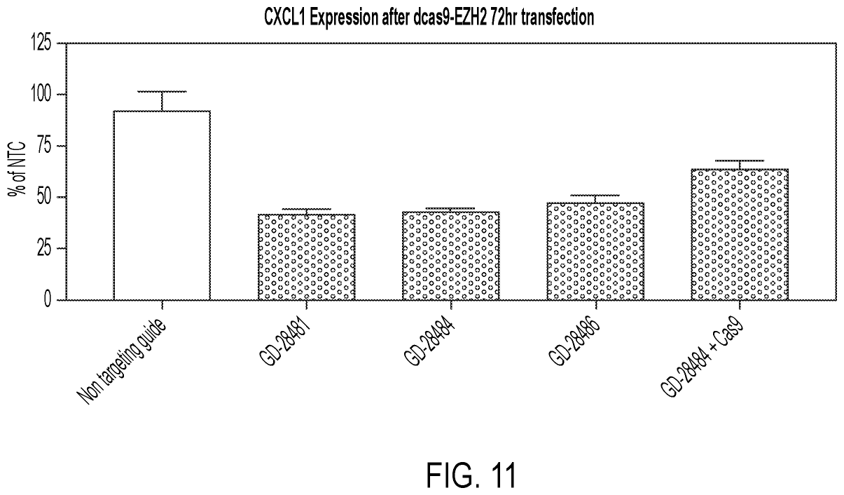

CXCL1 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 11) that a site-specific disrupting agent comprising a catalytically inactive CRISPR/Cas molecule fused to a histone methyltransferase (EZH2) and targeted to CTCF sites by different sgRNAs can be used to decrease CXCL1 expression in THP-1 cells. The decrease in expression was seen 72 hours post-transfection.

### Example 8: Agents Comprising MQ1 Effector Moieties Decrease CXCL1 Expression

This example describes, in part, experiments demonstrating a decrease in expression of CXCL1 in THP-1 cells after transfection with a site-specific disrupting agent comprising a CRISPR/Cas molecule fused to a DNA methyltransferase (MQ1) and a variety of guide RNAs targeted to the anchor sequences of the ASMC comprising the target plurality of genes.

THP-1 cells were grown in RPMI+10% FBS. Cells were transfected with mRNA encoding a catalytically inactive CRISPR/Cas molecule (dCas9) fused to MQ1, dCas9-MQ1, and sgRNA (from Example 1) targeted to either CTCF site of the ASMC comprising the target plurality of genes using LNPs. Lipid nanoparticle (LNP) formulation using SSOP lipid mixture was carried out by using the NanoAssemblr® Spark™ from Precision NanoSystems Inc. For experimental conditions, 350k cells/well were plated 72 hrs after LNP transfection for each transfected condition and the parental control into 24 well plates. One hour later 10 ng/ml of TNF alpha (Sigma Cat #654205) was added to each well. Untreated parental cells were plated with and without 10 ng/ml TNF alpha.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL1 and CXCL3 specific TaqMan primer/probe sets with the TaqMan® Fast Advanced Master Mix (Thermo Fisher Scientific).

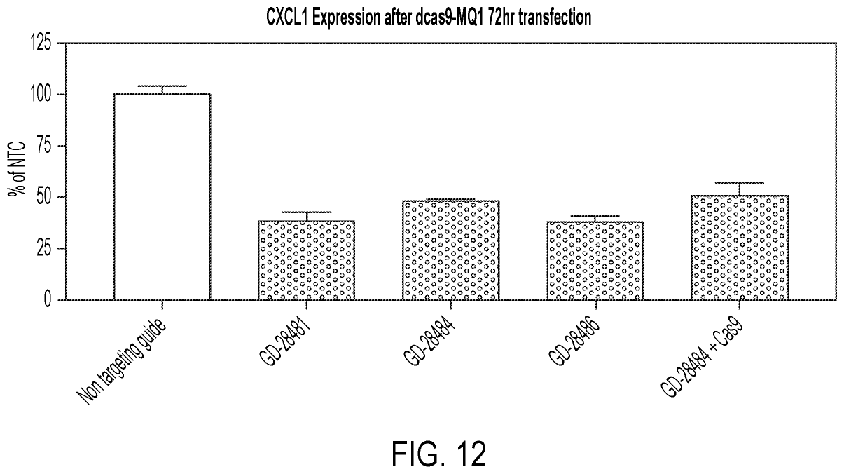

CXCL1 and 3 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 12) that a site-specific disrupting agent comprising a catalytically inactive CRISPR/Cas molecule fused to DNA methyltransferase (MQ1), and targeted to CTCF sites by different sgRNAs can be used to decrease CXCL1 expression in THP-1 cells. The decrease in expression was seen 72 hours post-transfection. Similar results were seen measuring CXCL3 expression (data not shown).

### Example 9: Durable CXCL1 Decrease in Expression after Cas9 or dCas9-EZH2 Treatment

This example describes, in part, experiments demonstrating a stable decrease in expression of CXCL1 in THP-1 cells up to 4 weeks after transfection with a site-specific disrupting agent comprising either a CRISPR/Cas molecule or a catalytically inactive CRISPR/Cas molecule fused to a histone deacetylase (EZH2) and an sgRNA targeted to an anchor sequence of the ASMC comprising the target plurality of genes comprising CXCL1.

Using the ATx™ Scalable Transfection System (MaxCyte), THP-1 cells grown in RPMI+10% FBS were electroporated with mRNA encoding either of the site-specific disrupting agents (Cas9 or dCas9-EZH2) and sgRNA (from Example 1) at 5 million cells per condition in processing assemblies. Samples of the transfected cells were harvested and incubated with TNF alpha for 24 hrs. This was repeated each week carried out to 4 weeks. After that time, DNA and RNA were isolated using the AllPrep DNA/RNA Kit (Qiagen) following the Manufacture's protocol. RNA samples were retrotranscribed to cDNA using LunaScript® RT SuperMix Kit (New England Biolabs) and analyzed by quantitative PCR using individual CXCL1, CXCL2, CXCL3 & IL8 specific TaqMan primer/probe sets with the TaqMan® Fast Advanced Master Mix (Thermo Fisher Scientific).

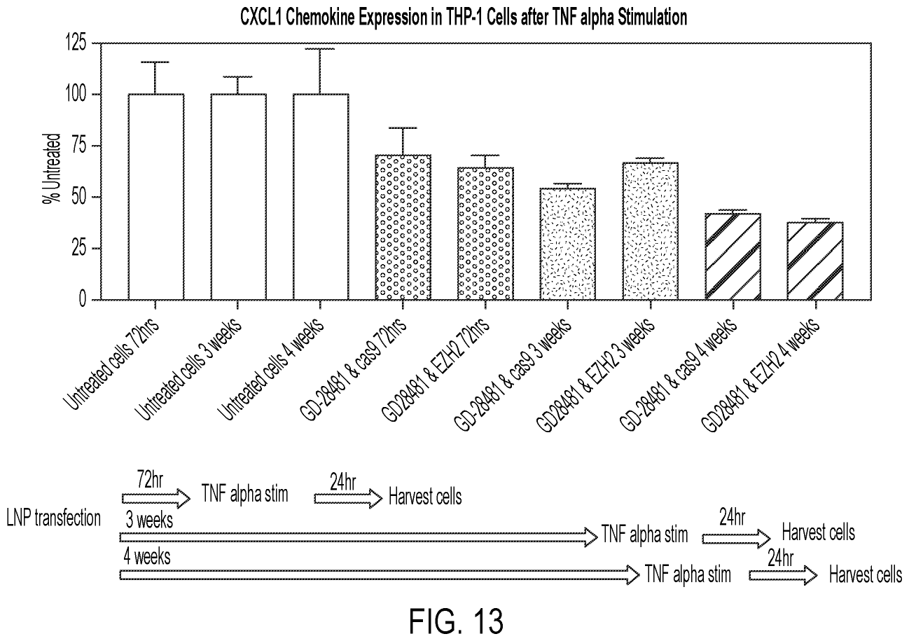

CXCL1 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show that (FIG. 13) a site-specific disrupting agent comprising a CRISPR/Cas molecule, or a catalytically inactive CRISPR/Cas molecule fused to a histone methyltransferase (EZH2) and targeted to a CTCF site by an sgRNA can be used to decrease CXCL1 expression in THP-1 cells. The decrease in expression is durable up to at least 4 weeks, and is also observed at 72 hours and 3 weeks post-transfection.

### Example 10: CXCL3 Expression Decreases Upon Treatment with EZH2-dCas9-KRAB and sgRNA

This example describes, in part, experiments demonstrating a decrease in expression of CXCL3 in THP-1 cells after transfection with a site-specific disrupting agent comprising a catalytically inactive CRISPR/Cas molecule fused to a histone methyltransferase (EZH2) and a transcriptional repressor (KRAB) and a variety of guide RNAs targeted to an anchor sequence of the ASMC comprising the target plurality of genes.

THP-1 cells were grown in RPMI+10% FBS. Several different site-specific disrupting agents were tested: G9A-dCas9-EZH2 (G9A fused to dCas9 fused to EZH2), G9A-dCas9-KRAB, and EZH2-dCas9-KRAB. Cells were transfected with mRNA encoding the site-specific disrupting agent, and sgRNA targeted to a CTCF site of the ASMC comprising the target plurality of genes using LNPs. The sgRNA was chosen to target a genomic DNA site proximal to the left CTCF site but some distance removed from the left CTCF site (e.g., 80, 160, 235, or 300 nucleotides from the CTCF site). Exemplary guide sequences targeting genomic DNA sites proximal to the left CTCF site, but some distance removed from the left CTCF site are given in Table 5.

TABLE 5

| Guide | Guide Sequence | Genomic Coordinates | SEQ ID No. |
|---|---|---|---|
| GD-29251 | CCAATGAAGATGAAACTGGG | chr4:74595215-74595237 | 30 |
| GD-29252 | AACGTGCTTGCCTAAGATTC | chr4:74595370-74595392 | 31 |
| GD-29253 | AGCCCTTAATCATATCTAGT | chr4:74595560-74595582 | 32 |
| GD-29254 | CAGAGCTTAAGACCTGTACT | chr4:74595642-74595664 | 33 |
| GD-29255 | GCCCACCTTGACCTTCACAA | chr4:74595787-74595809 | 34 |

Lipid nanoparticle (LNP) formulation using SSOP lipid mixture was carried out by using the NanoAssemblr© Spark™ from Precision NanoSystems Inc. For experimental conditions, 350k cells/well were plated 72 hrs after LNP transfection for each transfected condition and the parental control into 24 well plates. One hour later 10 ng/ml of TNF alpha (Sigma Cat #654205) was added to each well. Untreated parental cells were plated with and without 10 ng/ml TNF alpha.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL1 and CXCL3 specific TaqMan primer/probe sets with the TaqMan® Fast Advanced Master Mix (Thermo Fisher Scientific).

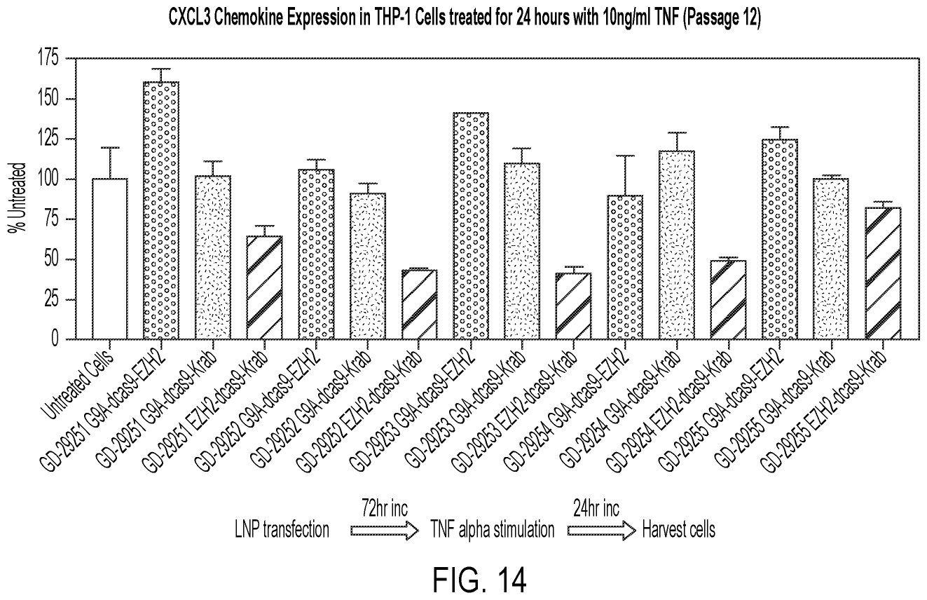

CXCL1 and 3 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 14) that a site-specific disrupting agent comprising a catalytically inactive CRISPR/Cas molecule fused to a histone methyltransferase (EZH2) and a transcriptional repressor (KRAB), and targeted to a site proximal to a CTCF an sgRNA can be used to decrease CXCL3 expression in THP-1 cells. Similar results were seen measuring CXCL1 expression (data not shown).

### Example 11: CXCL1 Expression Decreases Upon Treatment with Site-Specific Disrupting Agents and sgRNA

This example describes, in part, experiments demonstrating a decrease in expression of CXCL1 in THP-1 cells after transfection with various exemplary site-specific disrupting agents including: a catalytically inactive CRISPR/Cas molecule fused to a DNA methyltransferase (DNMT33a/3l); a catalytically inactive CRISPR/Cas molecule fused to a histone deacetylase (HDAC8); or a catalytically inactive CRISPR/Cas molecule fused to a histone deacetylase (HDAC8) and a histone methyltransferase (EZH2), and a variety of guide RNAs targeted to an anchor sequence of the ASMC comprising the target plurality of genes.

THP-1 cells were grown in RPMI+10% FBS. Several different site-specific disrupting agents were tested: dCas9-DNMT3a/3l (DNMT3a/3l fused to dCas9), dCas9-HDAC8, and EZH2-dCas9-HDAC8. Cells were transfected with mRNA encoding the site-specific disrupting agent, and sgRNA (from Example 1) targeted to either CTCF site of the ASMC comprising the target plurality of genes using LNPs. Lipid nanoparticle (LNP) formulation using SSOP lipid mixture was carried out by using the NanoAssemblr® Spark™ from Precision NanoSystems Inc. For experimental conditions, 350k cells/well were plated 72 hrs after LNP transfection for each transfected condition and the parental control into 24 well plates. One hour later 10 ng/ml of TNF alpha (Sigma Cat #654205) was added to each well. Untreated parental cells were plated with and without 10 ng/ml TNF alpha.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript© RT SuperMix Kit (New England Biolabs) and analyzed by quantitative PCR using individual CXCL1 specific TaqMan primer/probe sets with the TaqMan® Fast Advanced Master Mix (Thermo Fisher Scientific).

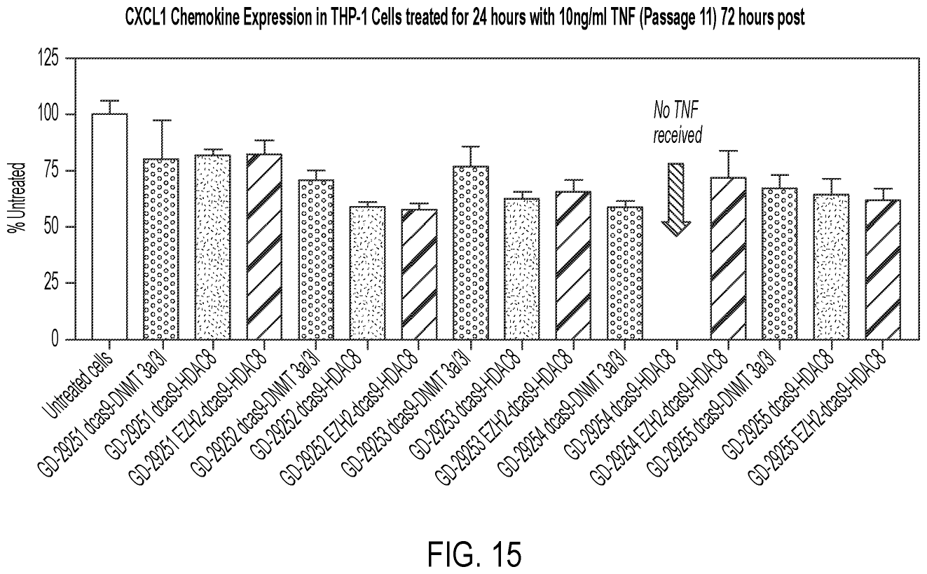

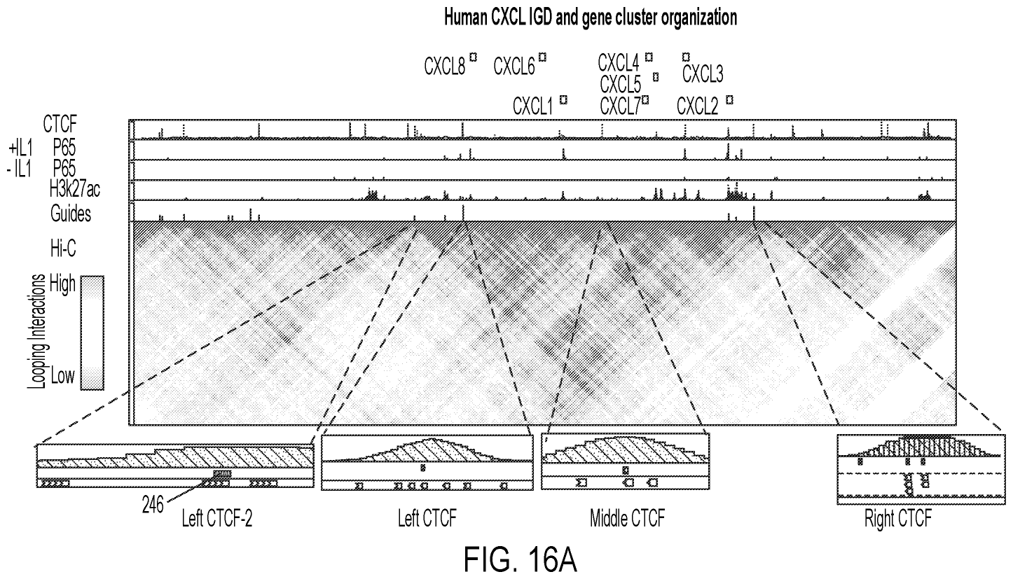

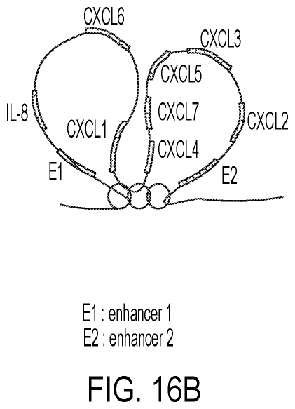

CXCL1 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 15) that a site-specific disrupting agent comprising dCas9-DNMT3a/3l, dCas9-HDAC8, or EZH2-dCas9-HDAC8 can be used to decrease CXCL1 expression in THP-1 cells and that these agents were effective at decreasing cytokine expression when targeted to CTCF sites by several different sgRNAs.

### Example 12: CXCL Gene Cluster Expression Decreases Upon Treatment with dCas9-EZH2 and Guide 30183 in Human A549 Lung Cancer Epithelial Cells and IMR-90 Cells

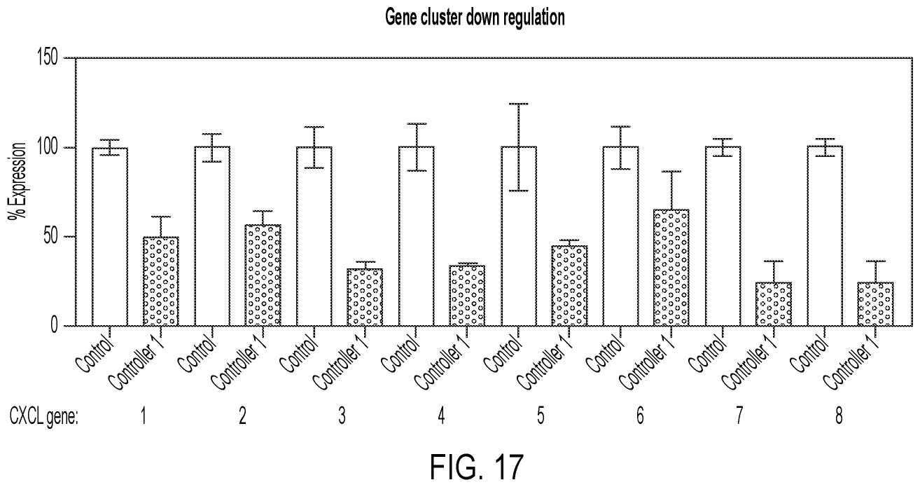

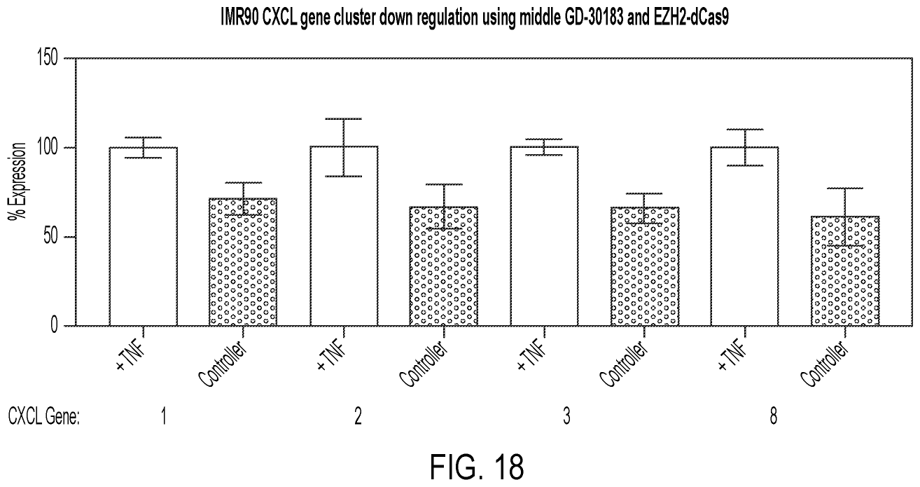

This example demonstrates CXCL gene cluster expression decreases in Human A549 lung cancer epithelial cells and IMR-90 cells when treated with dCas9-EZH2 and guide 30183 (Controller 1). Human A549 cells (ATCC© CCL-185) & IMR-90 cells (ATCC®-CCL-186) were plated at 15,000 cells per well in a flat bottom cell culture treated plate in 100 μl of media. A549 cells received F12/K ATCC©-30-2004 media and IMR-90 cells received EMEM ATCC®-30-2003 media. Both complete medias were made with 10% FBS (VWR cat #97068-085). After 24 hours adhering to the plate, LNPs containing guide 30183 and EZH2-dCas9 controller were added to the media at a final concentration of 2 g/ml SSOP lipid mix. After 6 hours, media was replaced with 100 μl of appropriate media and cells were incubated for 72 hours. After completion of 72-hour incubation, TNF alpha (Sigma Cat #654205) was added to designated wells at 10 ng/ml final concentration and incubated for 24 hours. After 24 hours, RNA was isolated using the NucleoSpin® 96 RNA Core Kit (Macherey-Nagel Inc, cat #740466.4) following the Manufacture's protocol. RNA samples were retrotranscribed to cDNA using LunaScript© RT SuperMix Kit (New England Biolabs) and analyzed by quantitative PCR using individual specific TaqMan primer/probe sets with the TaqMan® Fast Advanced Master Mix (Thermo Fisher Scientific). Gene expression was quantified relative to the human ABL1 reference gene using the ΔΔCt method. Changes in gene expression were further quantified by measuring the fold increase in gene expression after TNF alpha treatment directly compared to the levels of gene expression in the untreated cells. Data showed that expression of genes in CXCL gene cluster (specifically, CXCL1, CXCL2, CXCL3, CXCL4, CXCL5, CXCL6, CXCL7, and IL-8) levels were down between 40-70% in Human A549 lung cancer epithelial cells when treated with dCas9-EZH2 (FIG. 17). The expression of genes in CXCL gene cluster, (specifically, CXCL1, CXCL2, CXCL3, and IL-8) levels were down about 50% in IMR-90 cells when the middle CTCF was target with dCas9-EZH2 and GD-30183 (FIG. 18).

### Example 13: CXCL Gene Cluster Expression Decreases Upon Treatment with dCas9-EZH2 and Guide GD-28481 in Human Monocytes

This example demonstrates CXCL gene cluster expression decreases in Human monocyte cells when treated with dCas based effector (Controller A).

Transfection of the Cas9/guide RNP complex was carried out by electroporation into THP-1 cells (ATCC-TIB-202) by Synthego.

Upon receiving edited cell lines, vials were thawed, and cells were cultured in RPMI+10% FBS (VWR cat #97068-085) for one week to allow cells to recover from freezing and thawing. A parental unedited THP1 cell line was also analyzed for comparison. 350,000 cells were plated in quadruplicate for the edited cell line and the parental control into 24 well plates. One hour later 10 ng/ml of TNF alpha (Sigma Cat #654205) was added. Untreated control wells were also used to compare fold increase in chemokine expression.

The edited and parental cells were incubated with TNF alpha for 24 hrs. Afterwards, DNA and RNA were isolated using the DNA/RNA All Prep Kit (Qiagen) following the Manufacture's protocol. RNA samples were reverse transcribed to cDNA using LunaScript® RT SuperMix Kit (New England Biolabs) and analyzed by quantitative PCR using individual CXCL1, CXCL2, CXCL3 & IL8 specific TaqMan primer/probe sets with the TaqMan® Fast Advanced Master Mix (Thermo Fisher Scientific).

CXCL1-3 & IL8 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells.

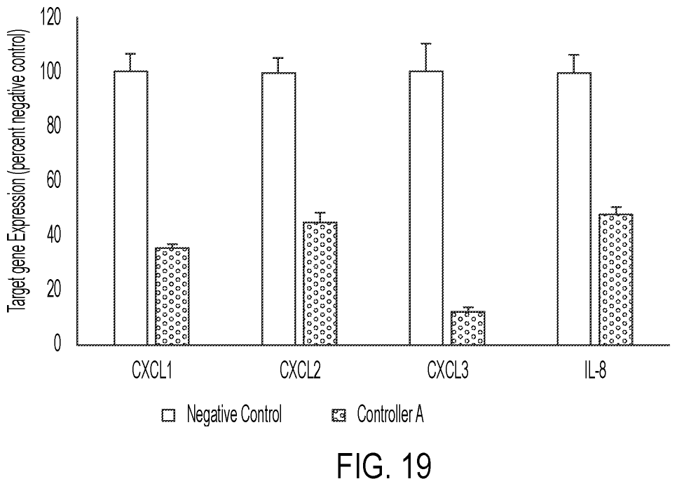

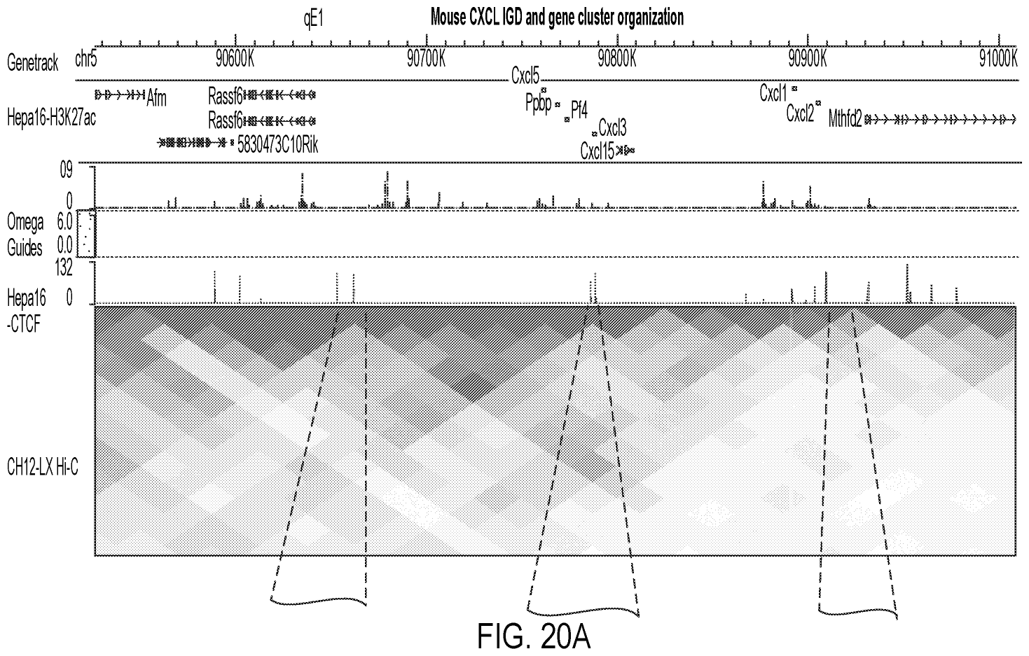

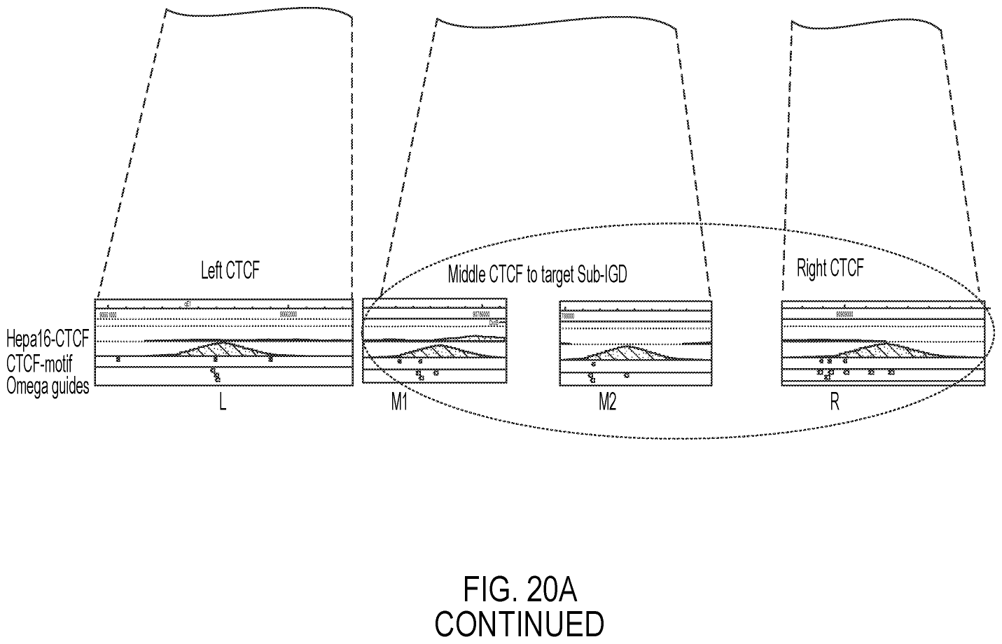

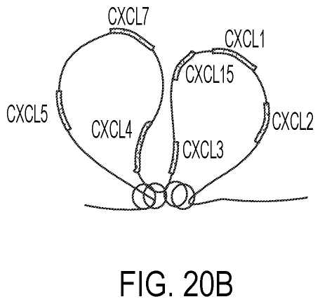

Data showed, at 24 hours post-dosing gene expression of CXCL1, CXCL2, CXCL3, and IL-8, decreased 65%, 55%, 88%, and 52% in monocytes treated with controller A compared to the CXCL1, CXCL2, CXCL3, and IL-8 gene expression respectively in untreated monocytes (FIG. 19).

### Example 14: Mouse CXCL Gene Cluster Expression Decreases Upon Treatment with dCas9-MQ1 and sgRNA Targeting Three Anchor Sequences in Hep 1.6 Cells

This example demonstrates mouse CXCL gene cluster expression downregulates when treated with dCas9-MQ1 and sgRNA targeting three anchor sequences in Hep 1.6 cells.

Mouse cells HEPA 1.6 (ATCC® CRL-1830) were plated at 10k cells per well in a flat bottom cell culture treated plate in 100 μl of media (DMEM Gibco Cat #11995-065, 10% FBS VWR cat #97068-085). After 24 hours adhering to the plate, the cultures were divided in four treatment groups and three control groups. LNPs containing (i) guides GD-30594 and dCas9-MQ1 controller targeting Right CTCF, (ii) guide GD-30592 with dCas9-MQ1 effector targeting middle CTCF 1, (iii) guide GD-30593 with dCas9-MQ1 effector targeting middle CTCF and (iv) a combination of GD-30594, GD-30592 and GD-30593 with dCas9-MQ1 targeting both middle and right CTCF were added to the cell cultures under treatment group at a final concentration of 2 μg/mL SSOP lipid mix. Untreated cells, cells treated with LNP, and cells treated with TNF and a LNP containing a transfection control guide were used as controls. After 6 hours, media was replaced with 100 μl of DMEM and cells were incubated for 72 hours. After completion of 72 hr incubation, TNF alpha (Sigma Cat #654245) is added to designated wells at 10 ng/ml final concentration and incubated for 24 hours. After 24 hrs, RNA was isolated using the NucleoSpin© 96 RNA Core Kit (Macherey-Nagel, cat #740466.4) following the Manufacture's protocol. RNA samples were retrotranscribed to cDNA using LunaScript® RT SuperMix Kit (New England Biolabs) and analyzed by quantitative PCR using individual specific TaqMan primer/probe sets with the TaqMan© Fast Advanced Master Mix (Thermo Fisher Scientific). Gene expression was quantified relative to the mouse HPRT reference gene using the ΔΔCt method. Changes in gene expression were further quantified by measuring the fold increase in gene expression after TNF alpha treatment directly compared to the levels of gene expression in the untreated cells.

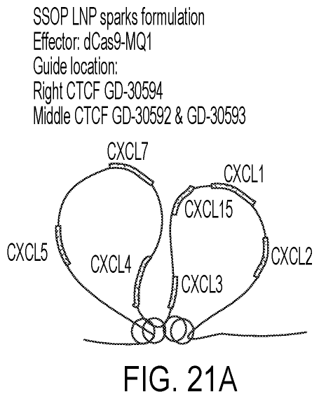

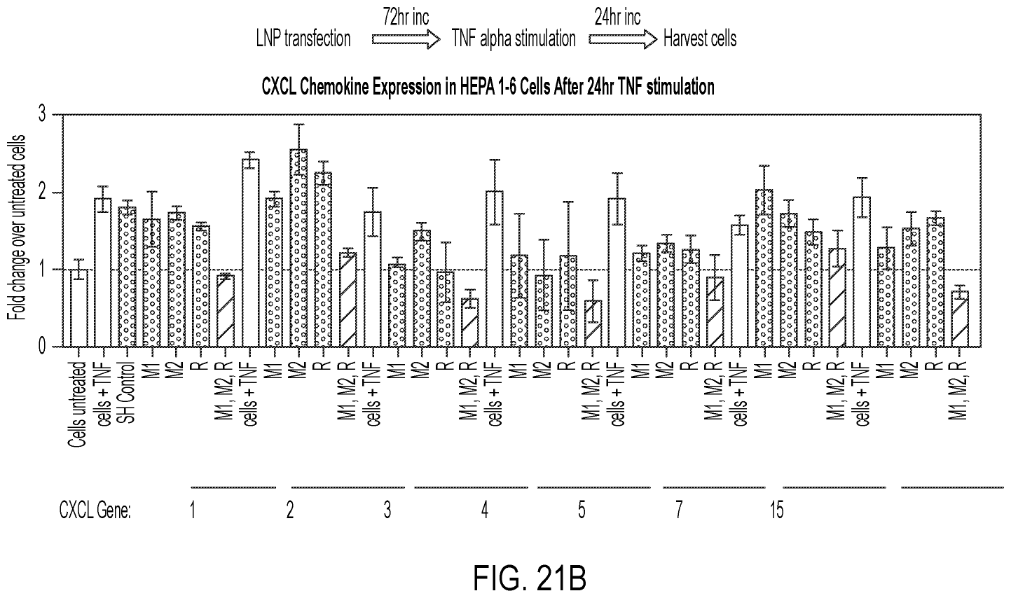

Data demonstrated, cells treated with dCas9-MQ1 transfected using guides targeting the right or one of the two middle CTCF motifs in the CXCL gene cluster, showed some down regulation in all of the seven CXCL genes after TNF alpha stimulation (FIG. 21B). However, the entire CXCL gene cluster was significantly more downregulated when cells were treated with dCas9-MQ1 transfected using a combination of guides targeting both middle and right CTCF (FIG. 21B).

### Example 15: Systemic Administration of dCas9-MQ1 Demonstrates a Significant Decrease in Leukocyte Infiltration in the Inflamed Lungs

This example demonstrates that systemic administration of dCas9-MQ1 decrease leukocyte infiltration in vivo in mouse lungs.

Murine lipopolysaccharide (LPS) lung inflammation model was used to study acute inflammation in the lungs. LNP comprising DOTAP 1% PEG short ncRNA was used as control. Each mouse received 3 mg/kg dose of LNP-DOTAP or dCas9-MQ1 at −2 hour time via intravenous administration point. The mice were stimulated with 5 mg/kg of LPS via oral aspiration at 0 hours. A second dose of LNP-DOTAP or dCas9-MQ1 at 3 mg/kg was administered at +8 hour time point. Dexamethasone was administered intraperitoneally at 10 mg/kg dose at times 0, 24, and 48 hours. The animals were terminated at 72 hours and bronchiolar lavage fluid were collected from the lungs for flow staining. Reduction in neutrophil infiltration in BALF was used a measure to understand the severity of inflammatory response.

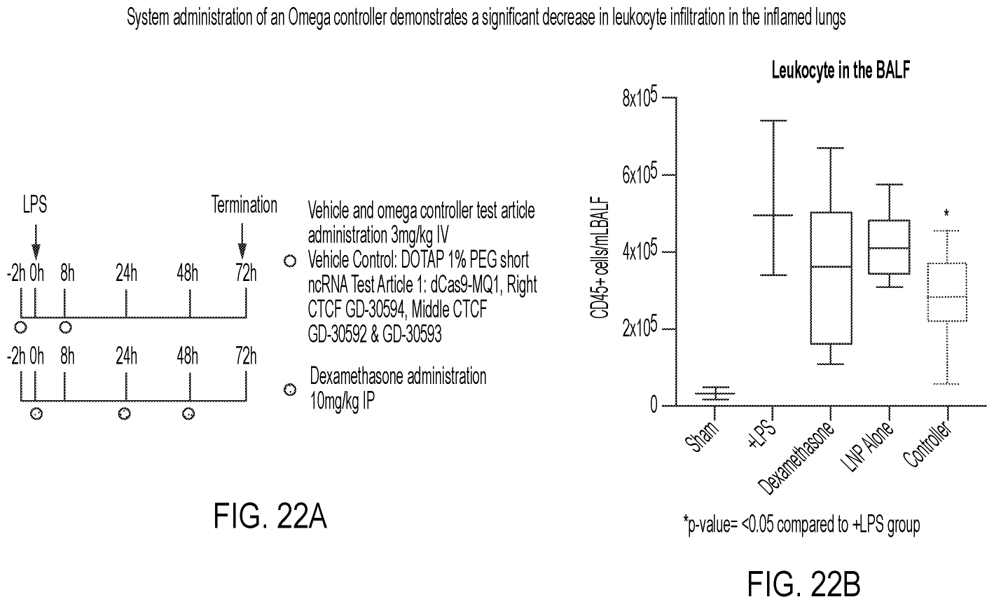

The bronchiolar lavage fluid collected from the lungs of dCas9-MQ1 treated animals showed about 5.0×10⁵ leukocyte count/mL. The sham group, i.e., healthy mice receiving no treatment did not have any significant presence of leukocyte in bronchiolar lavage fluid (BALF). The LPS treated mice, Dexamethasone treated mice, and LNP-DOTAP treated mice showed, about 8.0×10⁵ leukocyte count/mL, about 7.2×10⁵ leukocyte count/mL, and about 6.0×10⁵ leukocyte count/mL in the bronchiolar lavage fluid respectively (FIG. 22B). 56% decrease in neutrophils infiltration in bronchoalveolar lavage fluid (BALF) was also observed in mice 72 hours after treatment with dCas9-MQ1 compared to the disease control.

### Example 16: Systemic Administration of dCas9-MQ1 Demonstrates a Significant Decrease in Neutrophil Infiltration in BALF

This example analyzes BALF obtained in Example 15 to assess the cell population.

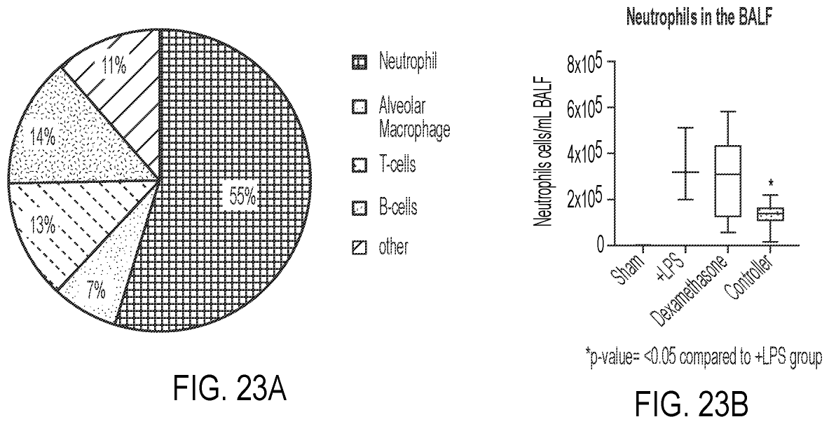

Flow cytometry analysis using the following staining panels below were used to assess the cell population in the BALF obtained in example 15 and the percentage of cells present in the BALF at the time of termination were documented (FIG. 23A). Neutrophil count in the BALF were also graphed using the antibody staining panel below.
alveolar macrophages: CD45⁺, Siglec F⁺, CD11b⁻, CD11c⁺
Neutrophils: CD45⁺, Siglec F⁻, CD11b⁺, CD11c⁻, Ly-6G⁺
T cells: CD45⁺, Siglec F⁻, CD1 1c⁻, CD3⁺
B cells: CD45⁺, Siglec F⁻−, CD11c⁻, B220⁺ (FIG. 23B)

Analysis showed, the leukocyte cell types that make up the majority of the infiltrating cells are neutrophils, followed by B cells, T cells, macrophages and other types of hematopoietic cells (FIG. 23A). The controller decreased the number of neutrophils infiltrating the lungs with significant difference compared to the +LPS disease group (FIG. 23B).

### Example 17: The Decrease of Leukocyte Cells in the BALF is Lung Specific

This example demonstrates that the reduction of Leukocyte cells in the BALF were lung specific suggesting the decrease was resulted from dCas9-MQ1 treatment.

Murine lipopolysaccharide (LPS) lung inflammation model was used to study acute inflammation in the lungs. LNP comprising DOTAP 1% PEG short ncRNA was used as control. Each mouse received 3 mg/kg dose of LNP-DOTAP or dCas9-MQ1 at −2 hour time via intravenous administration point. The mice were stimulated with 5 mg/kg of LPS via oral aspiration at 0 hours. A second dose of LNP-DOTAP or dCas9-MQ1 at 3 mg/kg was administered at +8 hour time point. Dexamethasone was administered intraperitoneally at 10 mg/kg dose at times 0, 24, and 48 hours. The animals were terminated at 72 hours and bronchiolar lavage fluid were collected from the lungs for flow staining. Peripheral blood was collected at 72 h termination. Flow analysis using CD45⁺ antibody staining was used to determine the leukocyte population in the peripheral blood in each group. The leukocyte count obtained for each group were plotted in a graph.

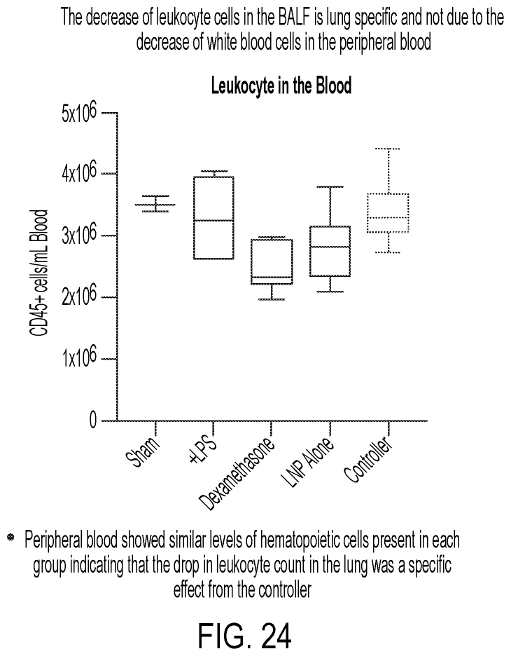

The graph illustrated that the effect of decreasing leukocyte count in the BALF with the controller treatment was lung specific suggesting the decrease in leukocyte count was due to dCas9-MQ1 treatment instead of the mouse itself had a decrease in leukocyte population which would have shown lower leukocyte count in peripheral blood as well. The hematopoietic cell population in the peripheral blood was found to be similar across all groups (FIG. 24).

### Example 18: Systemic Administration of dCas9-MQ1 Demonstrates CXCL Gene Expression is Decreased in the Lung Tissue

This example demonstrates CXCL gene cluster expression downregulates in lung tissue upon systemic administration of dCas9-MQ1.

BALF was collected using the method described in example 15. Following BALF collection, half of the left lung lobe was snapped frozen to store for qPCR analysis. The lung tissue was homogenized, and RNA was extracted for qPCR analysis quantifying specifically for CXCL1-7 and CXCL15. Gene expression was quantified relative to the mouse GAPDH reference gene using the ΔΔCt method.

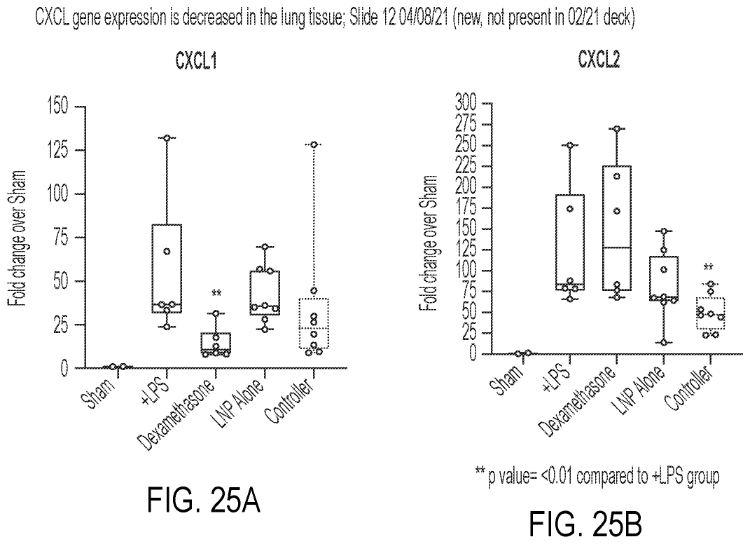

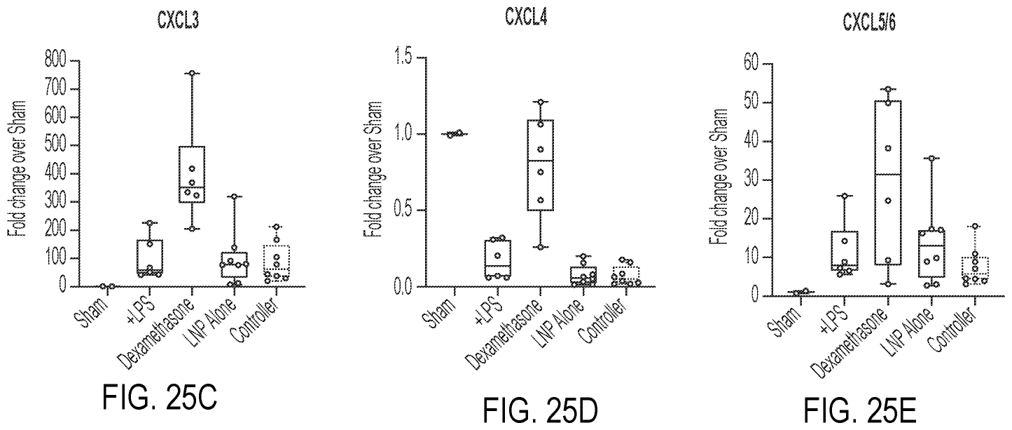

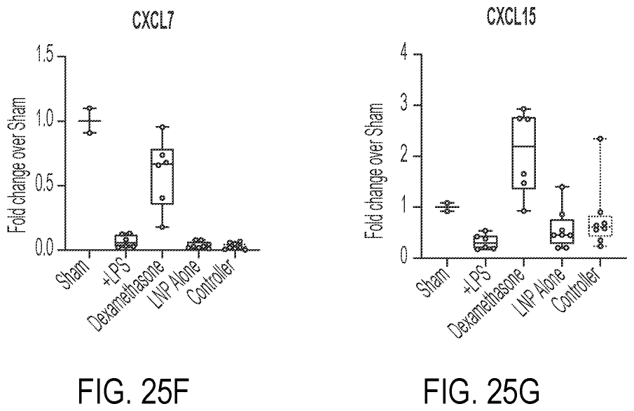

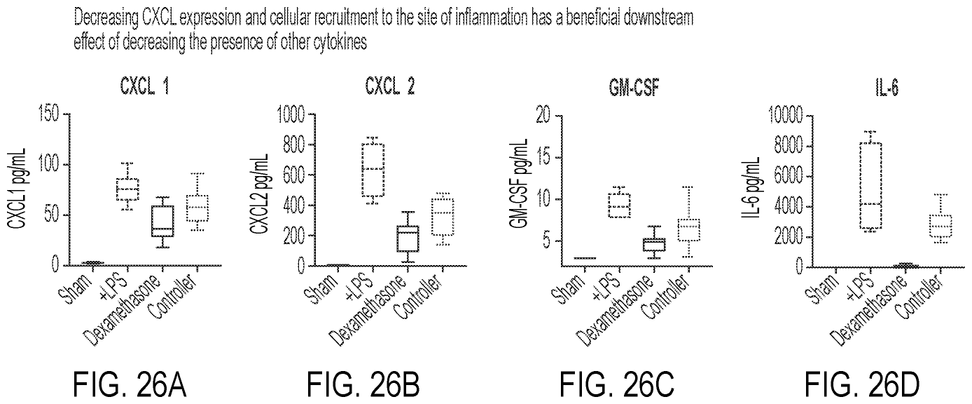

Data showed that the CXCL gene cluster expression was downregulated to varying extent upon in the lung tissue samples obtained from mice treated with dCas9-MQ1 compared to CXCL gene cluster expression in lung tissue samples obtained from mice that were not treated with dCas9-MQ1 (FIG. 25).

### Example 19: Decreasing CXCL Expression has a Beneficial Downstream Effect of Decreasing Cellular Recruitment and the Presence of Other Cytokines to the Site of Inflammation

Over-expression of the CXCL gene cluster produces chemokines that attract neutrophils. Chemokines that recruit inflammatory cells to the lung promote local inflammation, leading to severe pathogenesis. This example demonstrates downregulating CXCL expression has a beneficial downstream effect of reducing cellular recruitment leading to a reduction in the presence of other cytokines at the site of inflammation, suggesting downregulating CXCL expression is a promising method to reduce to the severity of inflammation pathogenesis.

BALF was collected using the method described in Example 15. Following BALF collection, half of the left lung lobe was snapped frozen to store for qPCR analysis. The lung tissue was homogenized, and RNA was extracted for qPCR analysis quantifying specifically for the total count of CXCL1, CXCL2, GM-CSF, and IL-6 protein in the BALF using multiplexing Luminex® instrument.

Data demonstrated that the lung tissues obtained from mice treated with dCas9-MQ1 showed a lower expression of CXCL1, CXCL2, GM-CSF, and IL-6 compared to the CXCL1, CXCL2, GM-CSF, and IL-6 expression found in the lung tissues obtained from mice that were not treated with dCas9-MQ1.

### Example 20: Decreasing Expression of an Exemplary Plurality of Genes

This example describes, in part, experiments demonstrating decreasing expression of a target plurality of genes comprising CXCL1, CXCL2, CXCL3, and IL8 using an expression repressor that targets a site in a E1 cis-acting regulatory element (cRE).

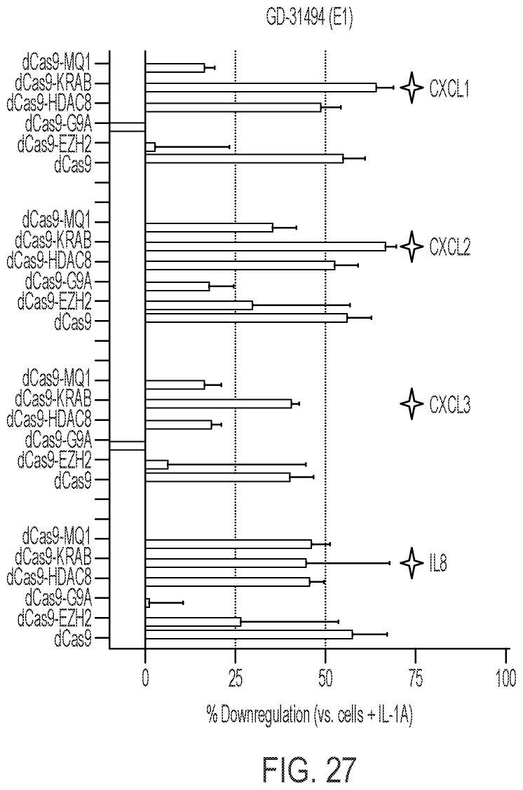

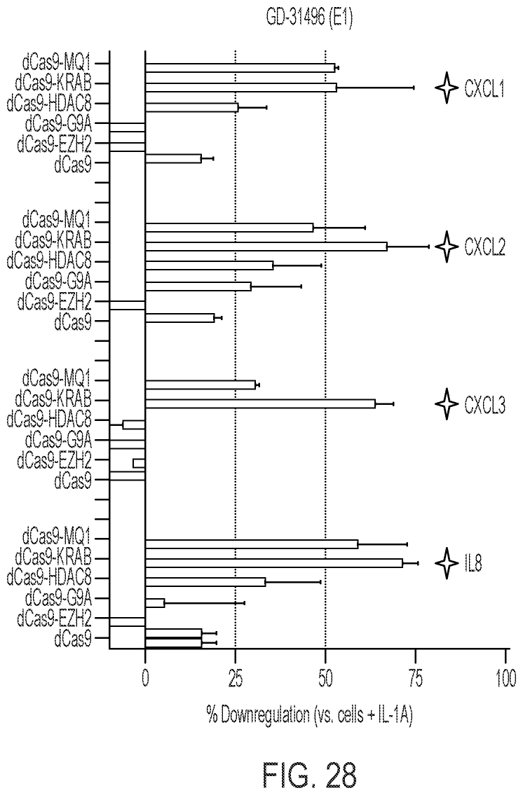

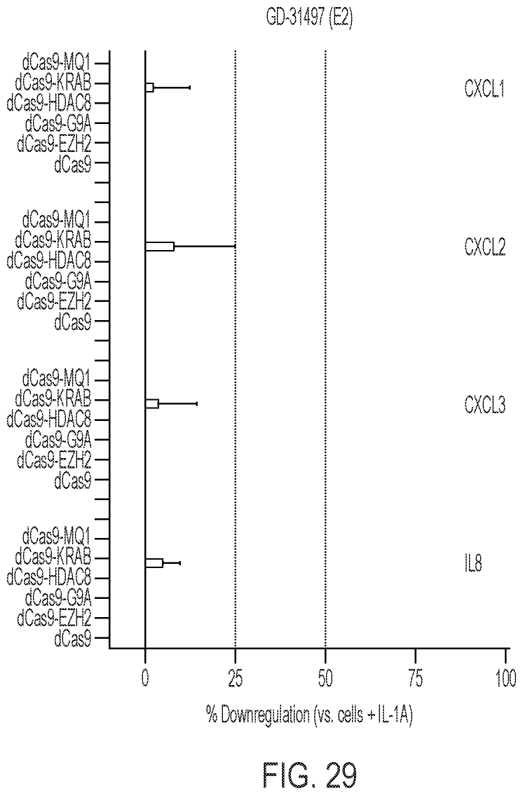

In this example, two sgRNA (GD31494 and GD31496, SEQ ID NOs: 102 and 104, respectively) complementary to a region in the enhancer E1 region of the Insulated Genomic Domain (IGD) and one sgRNA (GD31497, SEQ ID NO: 105) complementary to a region in the enhancer E2 region of the IGD were used in combination with 6 different expression repressors (dCas9-MQ1, dCas9-KRAB, dCas9-HDAC8, dCas9-G9A, dCas9-EZH2, and dCas9 control, as described herein) (FIGS. 27-29). The complete E1 cRE is 1,600 bp in size, located at coordinates chr4:74591400-74593000 (coordinates based on hg19 human genome reference assembly). The complete E2 cRE is 961 bp in size, located at coordinates chr4:74982639-74983600 (coordinates based on hg19 human genome reference assembly).

IMR-90 Cells (ATCCR CCL-186) were plated at 10k cells per well and transfected with SSOP LNPs (NOF Corporation) containing sgRNA and the expression repressor (e.g., dCas9-MQ1, dCas9-KRAB, dCas9-HDAC8, dCas9-G9A, dCas9-EZH2 or dCas9 (no effector)) mRNA at 1 g/ml LNP final concentrations for 48 hrs (approximately 0.5 g total mRNA). FIGS. 27-29 present CXCL1-3 and IL8 expression as seen after 1 hr of 10 ng/mL IL-1A stimulation. Percent gene downregulation is measured based on normalization to IL-1A stimulated cells. The data for FIGS. 27-29 are also presented in Tables 20-22.

TABLE 20
CXCL % downregulation by expression
repressor and E1 targeting GD31494

| | CXCL1 | CXCL2 | CXCL3 | IL8 |
|---|---|---|---|---|
| dCas9-MQ1 | 17 | 35 | 17 | 46 |
| dCas9-KRAB | 64 | 67 | 41 | 45 |
| dCas9-HDAC8 | 49 | 53 | 19 | 46 |
| dCas9-G9A | 0 | 18 | 0 | 1 |
| dCas9-EZH2 | 3 | 30 | 6.5 | 27 |
| dCas9 | 55 | 56 | 40 | 58 |

TABLE 21
CXCL % downregulation by expression
repressor and E1 targeting GD31496

| | CXCL1 | CXCL2 | CXCL3 | IL8 |
|---|---|---|---|---|
| dCas9-MQ1 | 53 | 47 | 31 | 59 |
| dCas9-KRAB | 54 | 68 | 64 | 72 |
| dCas9-HDAC8 | 26 | 36 | 0 | 34 |
| dCas9-G9A | 0 | 30 | 0 | 5 |
| dCas9-EZH2 | 0 | 0 | 0 | 0 |
| dCas9 | 16 | 19 | 0 | 16 |

TABLE 22
CXCL % downregulation by expression
repressor and E2 targeting GD31497

| | CXCL1 | CXCL2 | CXCL3 | IL8 |
|---|---|---|---|---|
| dCas9-MQ1 | 3 | 0 | 0 | 0 |
| dCas9-KRAB | 0 | 9 | 4 | 5 |
| dCas9-HDAC8 | 0 | 0 | 0 | 0 |
| dCas9-G9A | 0 | 0 | 0 | 0 |
| dCas9-EZH2 | 0 | 0 | 0 | 0 |
| dCas9 | 0 | 0 | 0 | 0 |

The data demonstrate that for CXCL1, dCas9-MQ1 resulted in approximately 20% downregulation in combination with GD31494, and greater than 50% downregulation in combination with GD31496. For CXCL2, dCas9-MQ1 resulted in an approximately 30% downregulation in combination with GD31494, and nearly 45% downregulation in combination with GD31496. For CXCL3, dCas9-MQ1 resulted in approximately 20% downregulation in combination with GD31494 and approximately 30% downregulation in combination with GD31496. For IL8, dCas9-MQ1 resulted in approximately 45% downregulation in combination with GD31494 and approximately 60% downregulation in combination with GD31496.

For CXCL1, dCas9-KRAB resulted in approximately 70% downregulation in combination with GD31494, and greater than 50% downregulation in combination with GD31496. For CXCL2, dCas9-KRAB resulted in an approximately 70% downregulation in combination with GD31494, and nearly 70% downregulation in combination with GD31496. For CXCL3, dCas9-KRAB resulted in approximately 40% downregulation in combination with GD31494 and approximately 70% downregulation in combination with GD31496. For IL8, dCas9-KRAB resulted in approximately 45% downregulation in combination with GD31494 and approximately 75% downregulation in combination with GD31496.

For CXCL1, dCas9-HDAC8 resulted in approximately 50% downregulation in combination with GD31494, and approximately 25% downregulation in combination with GD31496. For CXCL2, dCas9-HDAC8 resulted in a greater than 50% downregulation in combination with GD31494, and an approximately 40% downregulation in combination with GD31496. For CXCL3, dCas9-HDAC8 resulted in approximately 20% downregulation in combination with GD31494 and did not result in a significant decrease in expression in combination with GD31496. For IL8, dCas9-HDAC8 resulted in approximately 45% downregulation in combination with GD31494 and approximately 30% downregulation in combination with GD31496 for IL8.

For CXCL1, dCas9-G9A resulted in an increase in expression in combination with GD31494 and GD31496. For CXCL2, dCas9-G9A resulted in an approximately 20% downregulation in combination with GD31494, and a greater than 25% downregulation in combination with GD31496. For CXCL3, dCas9-G9A resulted in increase in expression in combination with GD31494 and GD31496. For IL8, dCas9-G9A did not result in a significant decrease in expression in combination with GD31494 or GD31496.

For CXCL1, dCas9-EZH2 did not result in a significant decrease in expression in combination with GD31494 or GD31496. For CXCL2, dCas9-EZH2 resulted in a greater than 25% downregulation in combination with GD31494, and an did not result in a significant decrease in expression in combination with GD31496. For CXCL3, dCas9-EZH2 did not result in a significant decrease in expression in combination with GD31494 or GD31496. For IL8, dCas9-EZH2 resulted in a greater than 25% downregulation in combination with GD31494 and an increase in expression in combination with GD31496.

For CXCL1, dCas9 resulted in a greater than 50% downregulation in combination with GD31494, and an approximately 20% downregulation in combination with GD31496. For CXCL2, dCas9 resulted in a greater than 50% downregulation in combination with GD31494, and an approximately 20% downregulation in combination with GD31496. For CXCL3, dCas9 resulted in an approximately 40% downregulation in combination with GD31494 and an increase in expression in combination with GD31496. For IL8, dCas9 resulted in a greater than 50% downregulation in combination with GD31494 and an approximately 20% downregulation in combination with GD31496.

Overall, this example demonstrates that numerous effectors targeted to two different sites in the E1 cRE are able to achieve downregulation of multiple genes near the E1 cRE.

### Example 21: Durability Study of IMR-90 Cells with Expression Repressors Targeting E1 cRE

This example describes, in part, experiments demonstrating the durability of expression repressors in decreasing expression of a target plurality of genes comprising CXCL1, CXCL2, CXCL3, and IL8 using an expression repressor that targets an E1 cis-acting regulatory element (cRE).

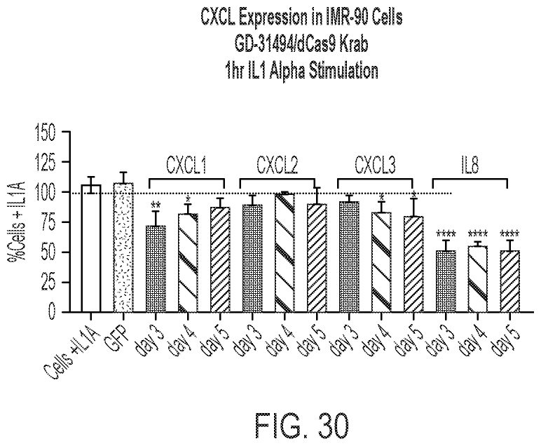

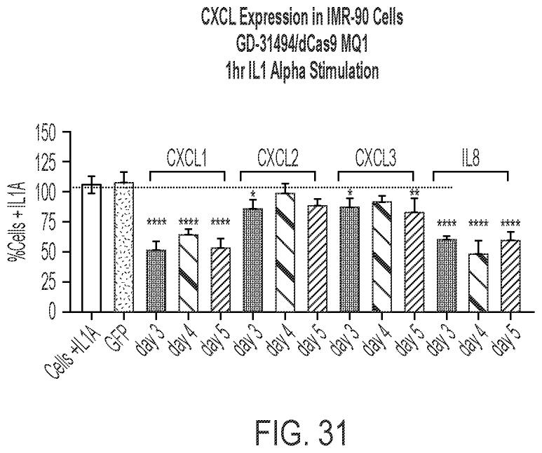

In this example, the sgRNA GD31494 (SEQ ID NO: 102) complementary to a site in the enhancer E1 region of the Insulated Genomic Domain (IGD) was used in combination with two different expression repressors (dCas9-MQ1 and dCas9-KRAB) (FIGS. 30 and 31).

IMR-90 Cells (ATCC® CCL-186) were plated at 10k cells per well and transfected with SSOP LNPs (NOF Corporation) containing sgRNA and the expression repressor (dCas9-MQ1 and dCas9-KRAB) mRNA (MR-28125 and MR-28122, SEQ ID NOs: 207 and 205, respectively) at 1 μg/ml LNP final concentrations for 48 hrs. FIGS. 30 and 31 shows downregulation of the CXCL gene cluster (e.g., CXCL1-3 and IL8) expression seen over 5 days when compared to the GFP treated control after 1 hr of 10 ng/mL IL-1A stimulation. Gene expression is measured as a fold change in mRNA expression over cells stimulated with IL-1A.

FIG. 30 and Table 23 show how dCas9-KRAB in combination with GD31494 resulted in greater than 25% downregulation in CXCL1 at day 3, and expression was still downregulated at day 5 when compared to control cells+IL-1A. CXCL2 was slightly downregulated at days 3 and 5 compared to control. dCas-KRAB with GD31494 induced decreased expression of CXCL3 through days 3-5. dCas9-KRAB in combination with GD31494 resulted in a nearly 50% downregulation of IL8 at days 3-5.

TABLE 23
CXCL % downregulation by expression repressor
and E1 targeting GD31494/dCas9-KRAB

| Time Course | CXCL1 | CXCL2 | CXCL3 | IL8 |
|---|---|---|---|---|
| Day 3 | 28 | 9.8 | 7.5 | 49 |
| Day 4 | 17 | 0.5 | 17 | 44 |
| Day 5 | 12 | 9 | 19 | 48 |

TABLE 24
CXCL % downregulation by expression repressor
and E1 targeting GD31494/dCas9-MQ1

| Time Course | CXCL1 | CXCL2 | CXCL3 | IL8 |
|---|---|---|---|---|
| Day 3 | 48 | 14 | 12 | 39 |
| Day 4 | 35 | 0.8 | 8 | 51 |
| Day 5 | 46 | 11 | 18 | 40 |

### Example 22: Durability Study of IMR-90 Cells with Expression Repressors Targeting E1 cRE

This example describes, in part, experiments demonstrating the durability of expression repressors in decreasing expression of a target plurality of genes comprising CXCL1, CXCL2, CXCL3, and IL8 using an expression repressor that targets an E1 cis-acting regulatory element (cRE).

In this example, the sgRNA GD31496 (SEQ ID NO: 104) complementary to a site in the enhancer E1 region of the Insulated Genomic Domain (IGD) was used in combination with two different expression repressors (dCas9-MQ1 and dCas9-KRAB) (FIGS. 32 and 33).

IMR-90 Cells (ATCC® CCL-186) were plated at 10k cells per well and transfected with SSOP LNPs (NOF Corporation) containing sgRNA and the expression repressor (dCas9-MQ1 and dCas9-KRAB) mRNA (MR-28125 and MR-28122, SEQ ID NOs: 207 and 205, respectively) at 1 g/ml LNP final concentrations for 48 hrs. FIGS. 32 and 33 shows downregulation of the CXCL gene cluster (e.g., CXCL1-3 and IL8) expression seen over 5 days when compared to the GFP treated control after 1 hr of 10 ng/mL IL-1A stimulation. Gene expression is measured as a fold change in mRNA expression over cells stimulated with IL-1A.

FIG. 32 and Table 25 show how dCas9-KRAB in combination with GD31496 resulted in greater than 25% downregulation in CXCL1 at day 3 and day 5 when compared to control cells+IL-1A. CXCL2 was slightly downregulated at day 5 compared to control. dCas9-KRAB with GD31496 induced decreased expression of CXCL3 through days 3-5, showing greater than 25% downregulation at day 5. dCas9-KRAB in combination with GD31496 resulted in a nearly 50% downregulation of IL8 at days 3-5.

TABLE 25
CXCL % downregulation by expression repressor
and E1 targeting GD31496/dCas9-KRAB

| Time | | | | |
|---|---|---|---|---|
| Course | CXCL1 | CXCL2 | CXCL3 | IL8 |
| Day 3 | 34 | −10 | 7.5 | 47 |
| Day 4 | 14 | −8 | 8 | 44 |
| Day 5 | 27 | 0 | 28 | 45 |

FIG. 33 and Table 26 show how dCas9-MQ1 in combination with GD31496 resulted 45% downregulation in CXCL1 at day 3, and 30% downregulation at days 4 and 5 when compared to control cells+IL-1A. CXCL2 was downregulated at day 5 compared to control. dCas-MQ1 with GD31496 induced decreased expression of CXCL3 through days 3-5. dCas9-MQ1 in combination with GD31496 resulted in a nearly 19% downregulation of IL8 at day 3, and greater than 24% downregulation at days 4 and 5.

TABLE 26
CXCL % downregulation by expression repressor
and E1 targeting GD31496/dCas9-MQ1

| Time | | | | |
|---|---|---|---|---|
| Course | CXCL1 | CXCL2 | CXCL3 | IL8 |
| Day 3 | 45 | −8 | 7 | 19 |
| Day 4 | 30 | −6 | 1.3 | 24 |
| Day 5 | 30 | −1 | 18 | 30 |

### Example 23: Durability Study of IMR-90 Cells with Expression Repressors Targeting the IL8 Promoter

This example describes, in part, experiments demonstrating the durability of expression repressors in decreasing expression of a target gene, IL8, using an expression repressor that targets the IL8 promoter.

In this example, the sgRNA GD31503 (SEQ ID NO: 111) complementary to a site in the IL8 promoter was used in combination with a dCas9-KRAB expression repressor (FIG. 34 and Table 27).

IMR-90 Cells (ATCCR CCL-186) were plated at 10k cells per well and transfected with SSOP LNPs (NOF Corporation) containing sgRNA complementary to a target site in the IL8 promoter and the expression repressor (dCas9-KRAB) mRNA (MR-28122, SEQ ID NO: 205) at 1 g/ml LNP. FIG. 34 shows downregulation of the IL8 expression up to 5 days after 1 hr of 10 ng/mL IL-1A stimulation on each specific day. Percent IL8 gene expression downregulation was calculated with normalization to IL-1A treated control.

TABLE 27
IL8 gene expression downregulation in IMR-90 cells
by an IL8 promoter targeting GD31503/dCas9-KRAB

| | % IL8 |
|---|---|
| Time Course | Downregulation |
| Day 3 | 91.1 |
| Day 4 | 84 |
| Day 5 | 62.1 |
| Day 6 | 21.5 |
| Day 7 | −4 |

### Example 24: E1 cRE Targeting Expression Repressors Demonstrate Robust Downregulation of CXCL Gene Cluster

This example describes, in part, experiments demonstrating decreasing expression of a target plurality of genes comprising CXCL1, CXCL2, CXCL3, and IL8 using an expression repressor that targets a site in an E1 cRE.

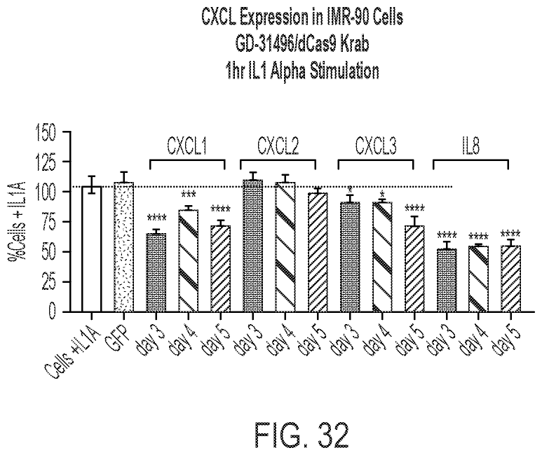

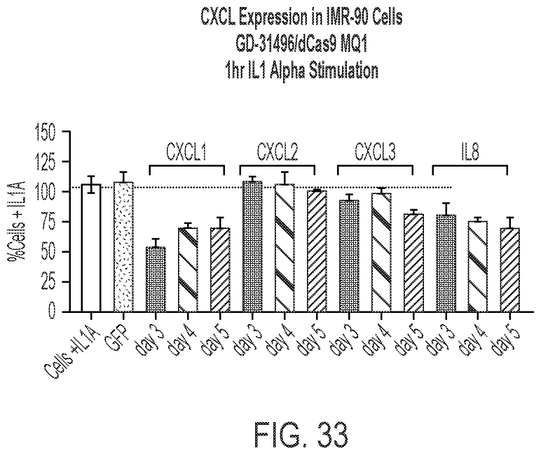

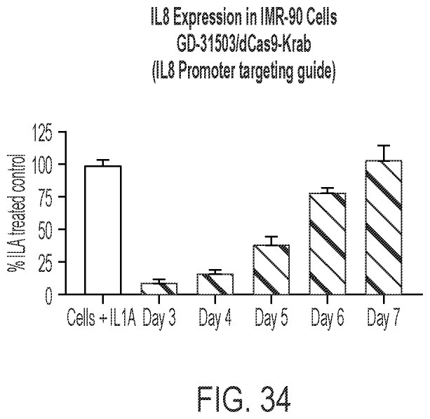

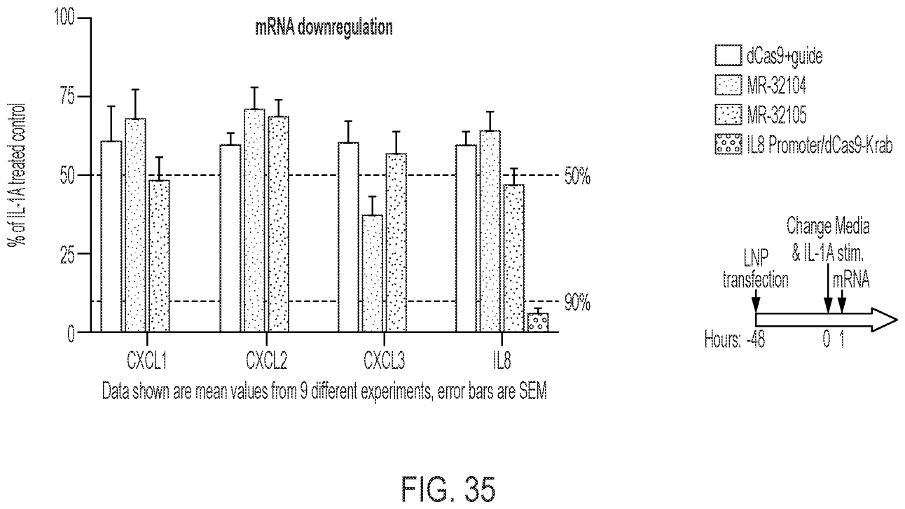

In this example, expression repressors including a zinc finger domain targeting moiety directed to the E1 cRE, and an expression repressor directed to the IL8 promoter (dCas-KRAB in combination with GD31503), were used to demonstrate robust downregulation of CXCL genes, such as CXCL1-3, and L8 (FIG. 35).

IMR-90 Cells (ATCCR CCL-186) were plated at 10k cells per well and transfected with SSOP LNPs (NOF Corporation) containing expression repressors (MR-32105, MR-32104, IL8 promoter targeting GD31503 with dCas9-Krab, and GD31496+dCas9-KRAB (dCas9+guide)) mRNA at 1 g/ml LNP final concentrations for 48 hrs. FIG. 35 and Table 28 present two expression repressors targeting the E1 cRE comprising a zinc finger domain targeting moiety and a KRAB effector moiety and a dCas9-KRAB expression repressor in combination with GD31503 directed to the IL8 promoter. More specifically, expression repressor MR-32104 has a sequence according to SEQ ID NO: 153 and comprises a zinc finger domain targeting the E1 region and a KRAB effector domain, and expression repressor MR-32105 has a sequence according to SEQ ID NO: 154 and comprises a zinc finger domain targeting the E1 region and a KRAB effector domain. Percent downregulation was calculated with normalization to IL1A treated control.

As seen in FIG. 35, MR-32015 down regulates CXCL1 and IL8 by greater than 50%, CXCL2 greater than 25%, and CXCL3 nearly 50%. MR-32104 downregulated CXCL1, CXCL2, and IL8 greater than 25%, and downregulates CXCL3 greater than 50%. dCas9-KRAB in combination with GD31496 downregulates IL8 greater than 90%.

### Example 25: E1 cRE Targeting Expression Repressors in Combination with IL8 Promoter Targeting Expression Repressors Demonstrate Robust Downregulation of CXCL Gene Cluster

This example describes, in part, experiments demonstrating decreasing expression of a target plurality of genes comprising CXCL1. CXCL2, CXCL3, and IL8 using a combination of expression repressors that targets a site in a E1 cis-acting regulatory element (cRE) and the IL8 promoter.

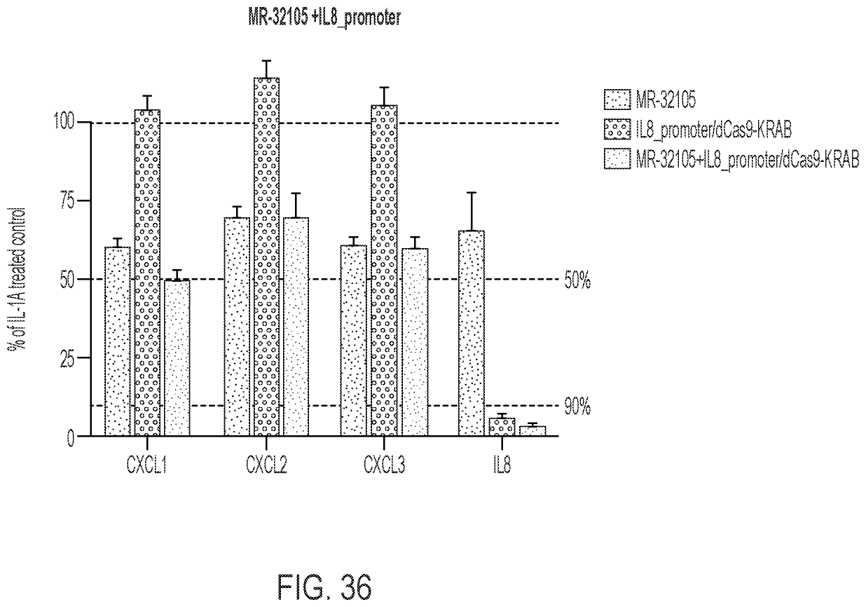

The data demonstrate the downregulation of the CXCL gene cluster using expression repressor MR-32105 directed to the E1 cRE in combination with IL8 promoter targeting dCas9-KRAB. FIG. 36 and Table 29 show gene down regulation across the CXCL1-3 and IL8 genes. The data further show that the MR-32105 expression repressor targeting E1 cRE and the dCas9-KRAB targeting the IL8 promoter do not interfere with one another, and that the combination of expression repressors has similar effect on IL8 compared to either expression repressor alone.

TABLE 29
CXCL % Downregulation

| mRNA | CXCL1 | CXCL2 | CXCL3 | IL8 |
|---|---|---|---|---|
| MR-32105 | 40 | 30 | 49 | 55 |
| GD-31503/dCas9-Krab | −4 | −14 | −5 | 94 |
| MR-32105 + GD-31503/dCas9-Krab | 50 | 30 | 40 | 97 |

### Example 26: E1 cRE Targeting Expression Repressors and IL8 Promoter Targeting Expression Repressors Demonstrate Robust Downregulation of IL8

This example describes, in part, experiments demonstrating decreasing expression of IL8 using expression repressors that target a site in an E1 cRE or the IL8 promoter.

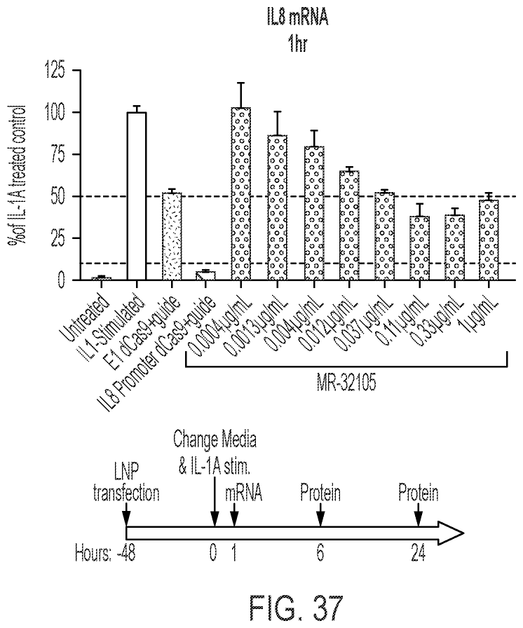

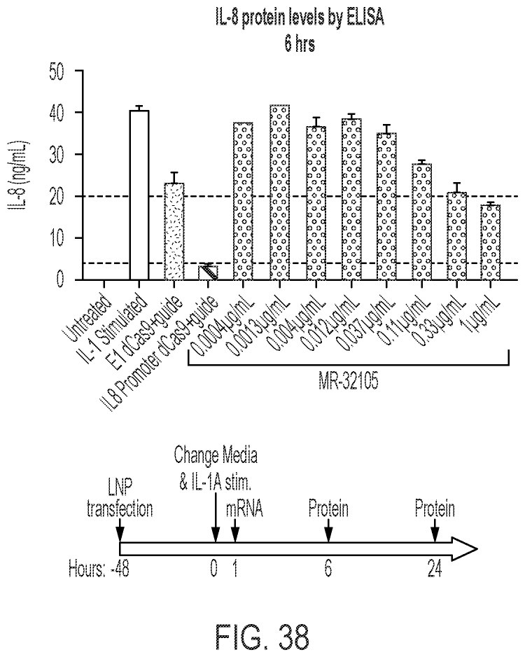

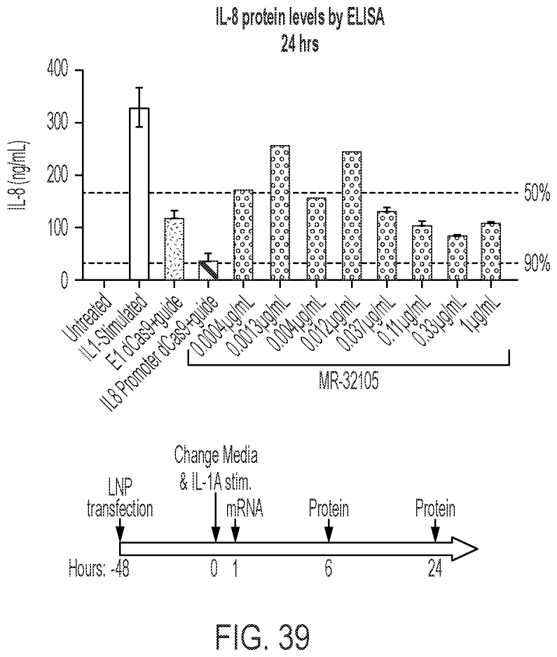

IMR-90 Cells (ATCCR CCL-186) were plated at 10k cells per well and transfected with 1 μg/ml-0.0004 μg/mL titration of SSOP LNPs (NOF Corporation) containing mRNA encoding expression repressor MR-32105 (e.g., an expression repressor having a sequence according to SEQ ID NO: 154 and comprises a zinc finger domain targeting the E1 region and a KRAB effector domain), IL8 promoter targeting dCas9-KRAB with GD31503, or dCas9-KRAB with GD31496 (dCas9+ guide) for 48 hrs. Percent downregulation was calculated with normalization to IL1A treated control (FIG. 37). For IL8 ELISA supernatants were collected 6 hrs or 24 hrs post IL-1A stimulation (FIGS. 38 and 39).

The data demonstrated robust down regulation of IL8 protein across varying concentrations of expression repressor. At 24 hours, MR-32105 induced greater than 50% downregulation for nearly all concentrations (0.0004 μg/ml, 0.004 g/ml, 0.037 g/ml, 0.11 g/ml, 0.33 μg/ml, and 1 g/ml) (FIG. 39). Furthermore, IL8 downregulation of greater than or equal to 90% was seen for mRNA and protein levels when treated with IL8 promoter targeting expression repressor (FIGS. 37-39).

### Example 27: Mechanism of Action of KRAB in Downregulating the CXCL Gene Cluster Using Expression Repressors MR-32104 and MR-32105

This example describes, in part, experiments demonstrating the on-target genomic mechanism of action of an E1 cRE targeting expression repressor with a KRAB effector. The data show increased histone methylation at the E1 cRE when a KRAB effector is targeted to the site.

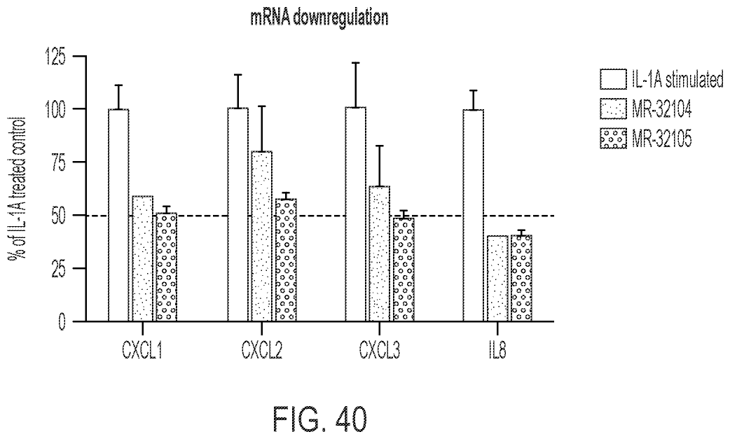

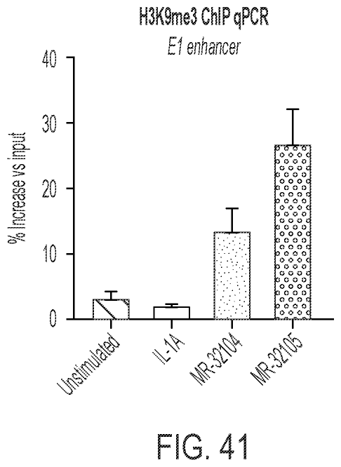

IMR-90 Cells (ATCC® k CCL-186) were plated at 4 million cells and transfected with 1 μg/ml SSOP LNPs (NOF Corporation) containing MR-32104 or MR-32105 for 48 hrs. 20K cells were pelleted for qPCR to quantify the downregulation levels of CXCL1-3 and IL8 (percent downregulation was calculated with normalization to IL1A treated control) (FIG. 40 and Table 30) before H3K9me3 CHIP was performed on IMR-90 cells control, stimulated with IL-1A, and transfected with 1 μg/ml SSOP LNPs (NOF Corporation) containing MR-32104 or MR-32105 for 48 hrs (FIG. 41). H3K9me3 CHIP was performed using ChIP-IT High Sensitivity (Catalog No. 53040), after treatment with MR-32105 or MR-32104 (Zinc Finger Domain-KRAB) an increase in H3k9me3 at the target enhancer (E1) was seen as expected. A higher increase of H3k9me3 after treatment with MR-32105 correlate with better downregulation of CXCL cluster.

TABLE 30
CXCL % Downregulation

| mRNA | CXCL1 | CXCL2 | CXCL3 | IL8 |
|---|---|---|---|---|
| MR-32104 | 41 | 19.2 | 36 | 60 |
| MR-32105 | 49 | 42 | 51 | 60 |

### Example 28: Durable Downregulation of CXCL Gene Clusters Induced by Expression Repressors

This example describes, in part, experiments demonstrating the durability of expression repressors in decreasing expression of a CXCL gene cluster using an E1 cRE targeting expression repressor with a KRAB effector.

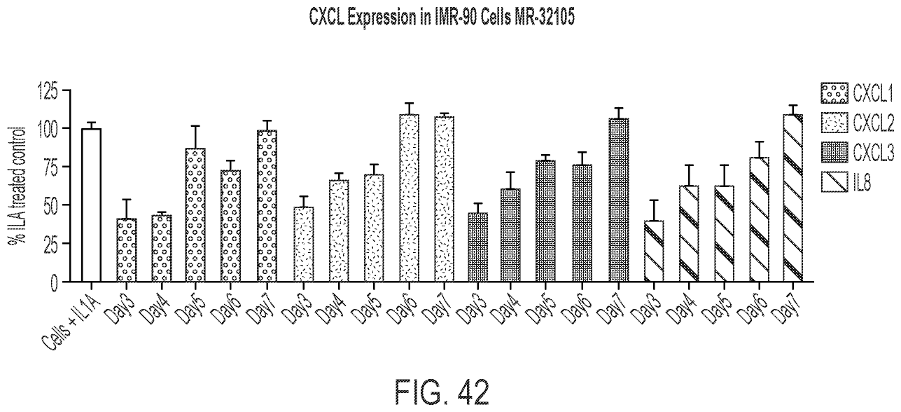

IMR-90 Cells (ATCC® CCL-186) were plated at 10k cells per well and transfected with SSOP LNPs (NOF Corporation) containing MR-32105 at 1 μg/ml LNP final concentrations (FIG. 42). FIG. 42 and Table 31 show the expression repressor MR-32105 induced downregulation of CXCL gene clusters. CXCL1-3 and IL8 gene expression remains decreased after 1 hr IL1A 10 ng/ml stimulation on each specific day. Percent CXCL1-3 and IL8 gene downregulation was calculated with normalization to IL-1A treated control.

TABLE 31
MR-32105 induced CXCL % Downregulation

| Time Course | CXCL1 | CXCL2 | CXCL3 | IL8 |
|---|---|---|---|---|
| Day 3 | 59 | 52 | 56 | 61 |
| Day 4 | 57 | 34 | 39 | 36 |
| Day 5 | 13 | 30 | 21 | 38 |
| Day 6 | 28 | −9 | 24 | 19 |
| Day 7 | 1 | −8 | −6 | −9 |

### Example 29: Monocistronic IL8 Promoter Expression Repressor Screen Shows Downregulation with ZF and TAL KRAB without Downregulating Other CXCL Genes

This example describes, in part, experiments demonstrating decreasing expression of IL8 without decreasing expression of other CXCL genes using expression repressors that target a site in the IL8 promoter.

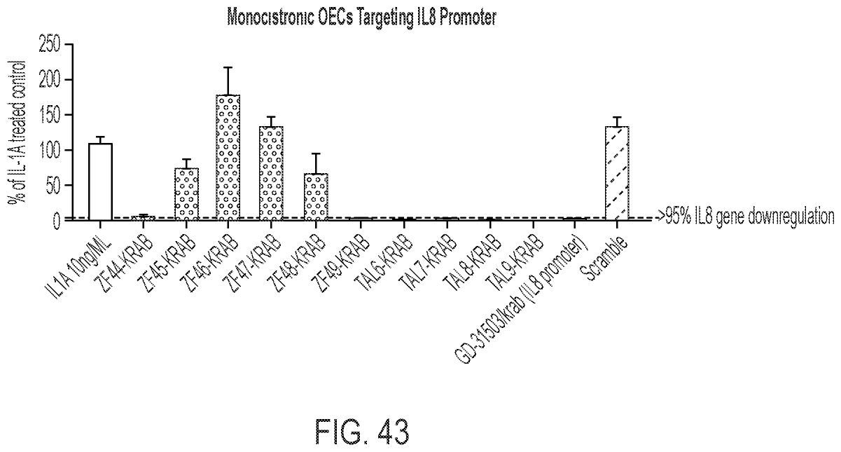

Human IMR90 Cells were used to measure the down regulation of CXCL gene expression upon IL1A stimulation. IMR-90 Cells (ATCC® CCL-186) were plated at 30k cells per well and transfected with SSOP LNPs (NOF Corporation) containing Zinc Finger-KRAB or Tal-KRAB mRNA for 48 hrs as seen in FIG. 43. In particular, FIG. 43 shows the effects on IL8 levels of ZF44-KRAB, ZF45-KRAB, ZF46-KRAB, ZF47-KRAB, ZF48-KRAB, ZF49-KRAB (see Tables 13-15) and TAL6-KRAB, TAL7-KRAB, TAL8-KRAB, TAL9-KRAB (see Tables 10-12), as well as GD-31503/KRAB. Final LNP concentration was 0.5 g/ml. Cells were stimulated with 10 ng/ml IL1A for 1 hr. The Zinc Fingers and Tals were designed to target the IL8 promoter region of the IGD to influence gene expression. FIG. 43 shows a decrease in IL8 Expression after 1 hr IL1A 10 ng/ml stimulation. Gene Expression is measured as % expression of IL1A treated cells.

### Example 30: E1-Targeting Expression Repressor is Active at the Target Site

This example describes, in part, experiments demonstrating the molecular activity of the E1-targeting expression repressor (MR-32105).

In this example, MR-32105 was applied to lung fibroblast cells (IMR90). MR-32105 assessments were made to evaluate (1) intended expression repressor/chromatin association at the target binding site, (2) intended change in epigenomic state near the target binding site, and (3) intended decrease of P65 transcription factor at the target enhancer.

IMR-90 cells (ATCC® CCL-186) were grown and treated with HA-tagged versions of MR-32105 (in SSOP LNP at 2 μg/mL). First, 24 hours post expression repressor treatment, cells were stimulated with IL1A for 1 hour then harvested, cells were assessed for MR-32105 controller/chromatin association by chromatin immunoprecipitation using HA-ChIP-seq. To evaluate epigenomic changes, 48 hours post treatment cells were stimulated with IL1A for 1 hour then harvested. The epigenomic activity was confirmed by measuring the increase in histone methylation using chromatin immunoprecipitation (H3K9me3-ChIP-seq) and the decrease in histone acetylation using chromatin immunoprecipitation (H3K27ac-ChIP-seq) at the target locus. To evaluate P65 transcription factor at the target enhancer, 48 hours posttreatment cells were stimulated with IL1A for 1 hour then harvested. P65 transcription factor depletion at the target E1 was evaluated using chromatin association at the target enhancer using P65-ChIP-seq.

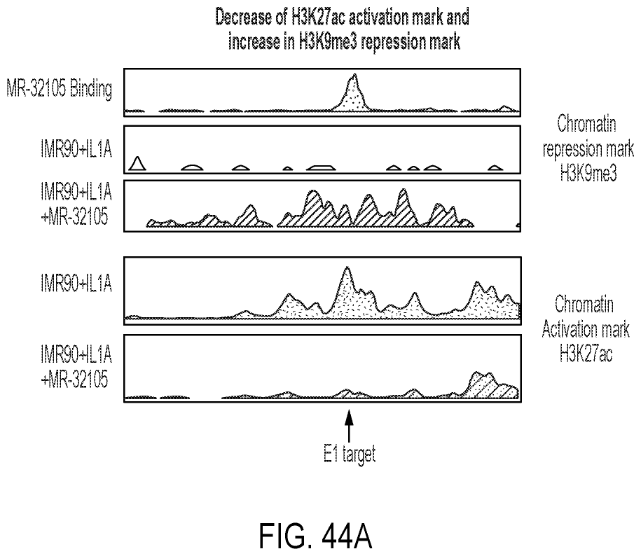

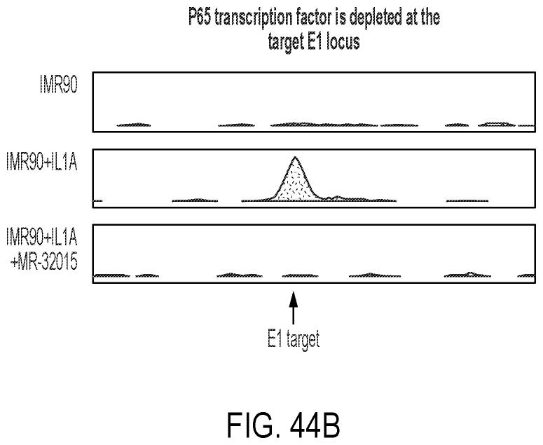

HA-ChIP-seq analysis revealed on-target enrichment for E1-targeting expression repressor. A significant differential increase in on-target DNA histone methylation (H3K9me3) and decrease in on-target histone acetylation (H3K27ac) was observed following MR-32105 treatment, leading to a significant decrease in on-target P65 (FIG. 44A-44B).

### Example 31: MR-32104 and MR-32105 Downregulate CXCL Cluster Relative to IL1-Alpha Stimulated Cells

This example describes, in part, experiments demonstrating decreased expression of CXCL1, CXCL2, CXCL3 and IL8 in IMR-90 Cells (ATCC® CCL-186) following treatment of expression repressors.

IMR-90 cells were cultured and plated (10k cells/well) in EMEM (ATCC-30-2003). Complete media was made with 10% FBS (VWR cat #97068-085).

IMR-90 cells were transfected with SSOP LNPs (NOF Corporation) containing MR-32104 or MR-32105 mRNA which were added to the media at a final concentration of 0.2 g/ml SSOP lipid mix.

After completion of 48 hr incubation, IL1-alpha (Life Technologies Cat #PCH0015) was added at ng/ml final concentration and allowed to incubate for 1 hour.

After the 1 hr incubation with IL1-alpha, RNA was isolated using the Macherey-Nagel Inc. RNA extraction kit (cat #740466.4) following the Manufacture's protocol. RNA samples were retrotranscribed to cDNA using LunaScript RT SuperMix Kit (New England Biolabs) and analyzed by quantitative PCR using individual specific Taqman primer/probe sets with the Taqman Fast Advanced Master Mix (Thermo Scientific).

Gene expression was quantified relative to the human ABL1 reference gene using the ΔΔCt method. Changes in gene expression were further quantified by measuring the fold increase in gene expression after IL1 alpha treatment directly compared to the levels of gene expression in the untreated cells.

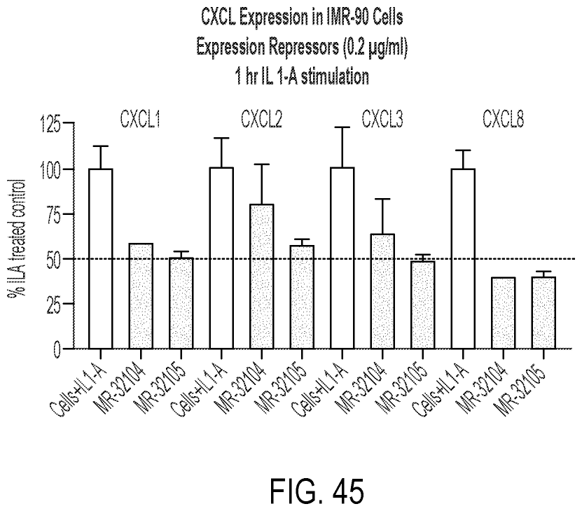

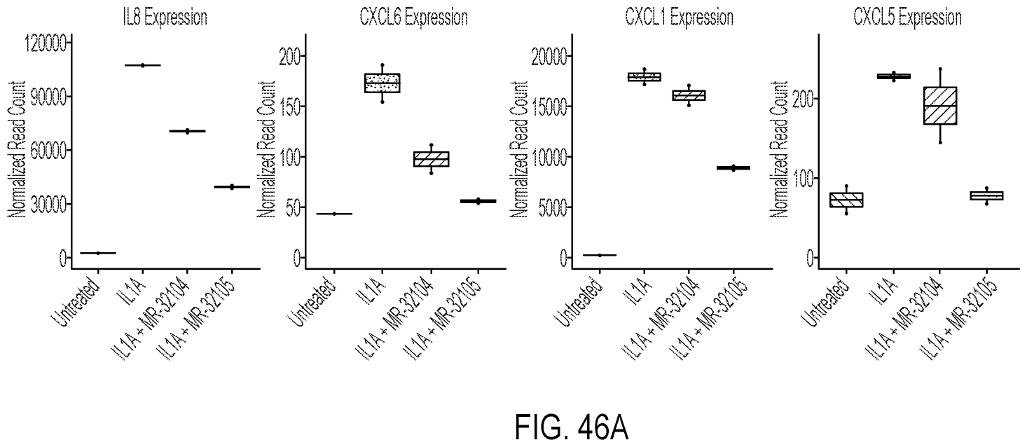

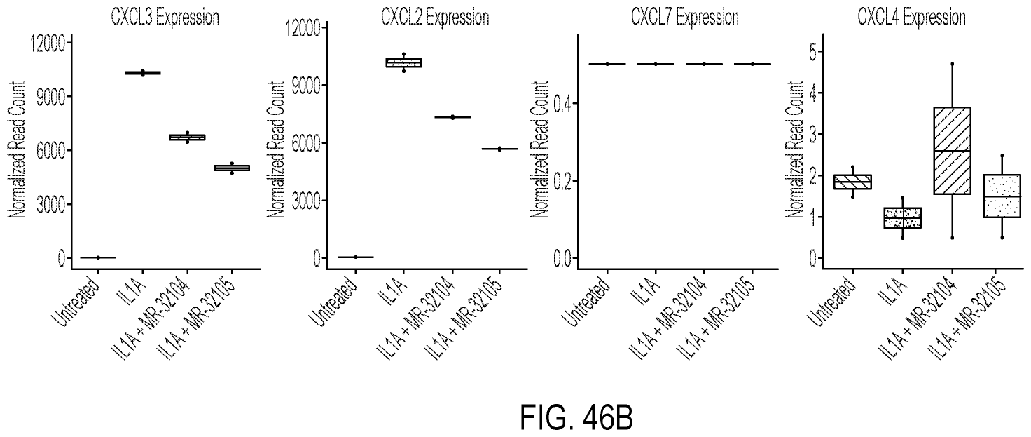

The results show that targeting the E1 enhancer region using MR-32105 or MR-32104 can be used to decrease CXCL1, CXCL2, CXCL3, and IL8 expression in IMR-90 cells about 40-60% (FIG. 45) and that expression was decreased at 48 hours post-treatment. MR-32105 also reduces expression of CXCL6 and CXCL5 (FIGS. 46A and 46B). MR-32104 also reduces expression of CXCL6 (FIGS. 46A and 46B). No effect was seen in CXCL4 and CXCL7 upon MR-32104 or MR-32105 treatment since these genes do not exhibit initial upregulation after IL1A stimulation in IMR90 cells. The data are shown in FIGS. 45 and 46A-46B.

### Example 32: IL8-Targeting Expression Repressor is Active at the Target Site

This example describes, in part, experiments demonstrating the molecular activity of an IL8-targeting expression repressor. In this example, MR-32712 assays were applied to lung fibroblast cells (IMR90). MR-32712 assessments were made validating (1) intended expression repressor/chromatin association at the target binding site, (2) intended change in epigenomic state near the target binding site, and (3) intended decrease of P65 transcription factor at the target enhancer (4) intended decrease in IL8 transcription activity

IMR90 cells were grown, treated with MR-32712, 24 hours post treatment cells were stimulated with IL1A for 1 hour then harvested, Cells were assessed for (1) MR-32712 expression repressor/chromatin association by chromatin immunoprecipitation using HA-ChIP-seq, (2) epigenomic activity by measuring increase in histone methylation using chromatin immunoprecipitation (H3K9me3-ChIP-seq) at the target IL8 locus, (3) validation the decrease of P65 transcription factor chromatin association at the target enhancer using P65-ChIP-seq, and (4) transcription activity OF IL8 by RNA-qPCR and RNA-seq

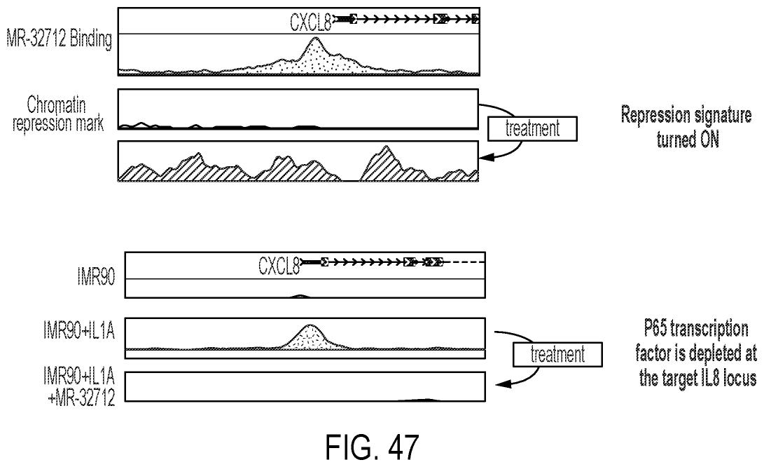

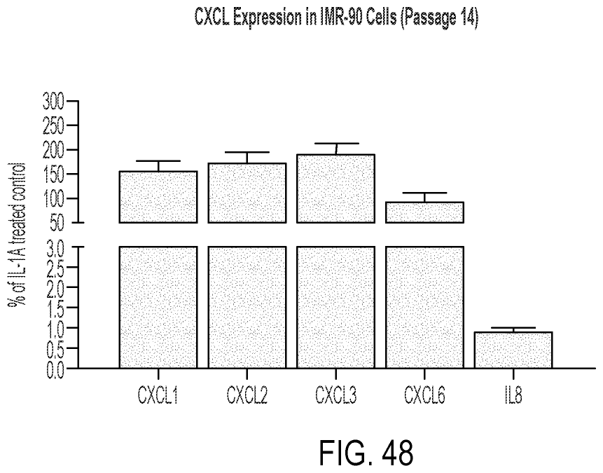

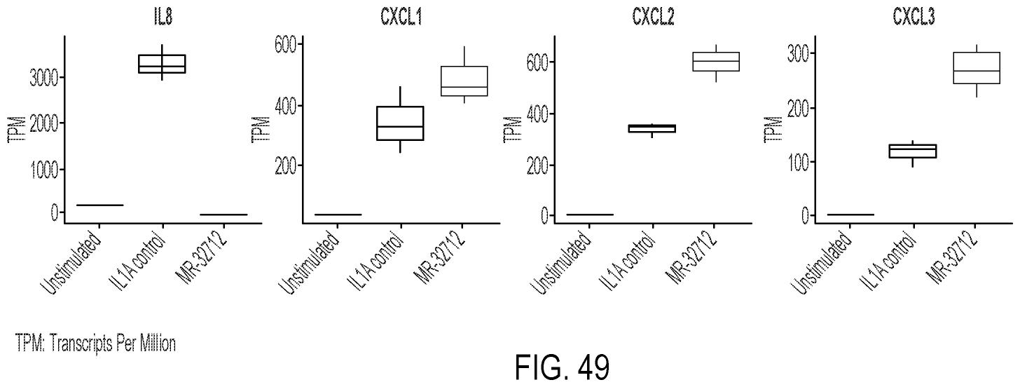

HA-ChIP-seq analysis revealed on-target enrichment for MR-32712. A significant differential increase in on-target DNA histone methylation was observed following MR-32712. A significant decrease in on-target P65 as observed following treatment, which led to ˜95% decrease of IL8 expression and some increase for other CXCL within CXCL1-8 cluster based on RNA-qPCR and RNA-seq (FIGS. 47-49).

### Example 33: Expression Repressor Protein Depletion after 24 and 48 Hours

This example describes, in part, experiments demonstrating when the expression repressors will deplete from engagement with the target site.

IMR90 cells were grown, treated with HA-tagged versions of MR-32105, stimulated for 1 hour with IL1A after 24 and 48 hours then harvested. MR-32105 was detected using controller/chromatin association by chromatin immunoprecipitation using HA-ChIP-qPCR.

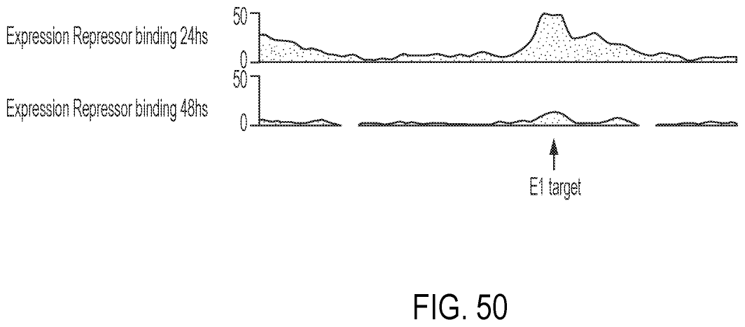

HA-ChIP-qPCR analysis revealed on-target enrichment for the E1-targeting expression repressor at 24 hours but no detectable signal at the target or anywhere else in the genome at 48 hours (FIG. 50).

### Example 34: CXCL1-8 Expression Downregulated in Small Airway Epithelial Cells (COPD)

This example describes an experiment demonstrating decreased expression of CXCL1, CXCL2, CXCL3 and IL8 in Small airway epithelial cells (HSAEpC) (Promocell C-12642 lot #435Z002) from a patient diagnosed with COPD type II.

HSAEpC cells were transfected with MR-32905 added to the media at a final concentration of 1 μg/ml SSOP lipid mix.

After completion of 48 hr incubation, IL1-alpha (Life Technologies Cat #PCH0015) was added at ng/ml final concentration and were incubated for 1 hour.

After the 1 hr incubation with IL1-alpha, RNA was isolated using the Macherey-Nagel Inc RNA extraction kit (cat #740466.4) following the manufacturer's protocol. RNA samples were retrotranscribed to cDNA using LunaScript RT SuperMix Kit (New England Biolabs) and analyzed by quantitative PCR using individual specific Taqman primer/probe sets with the Taqman Fast Advanced Master Mix (Thermo Scientific).

Gene expression was quantified relative to the human GAPDH reference gene using the ΔΔCt method. Changes in gene expression were further quantified by measuring the fold increase in gene expression after IL1-alpha treatment directly compared to the levels of gene expression in the untreated cells.

Protein levels were determined using the Thermo Fisher Luminex FlexMAP 3D Multiplexing Microplate reader. Supernatant was collected after 1 hr incubation with IL1-Alpha and cytokines were measured using the ProcartaPlex 7 plex kit (cat #PPX-07-MXT2AW2.

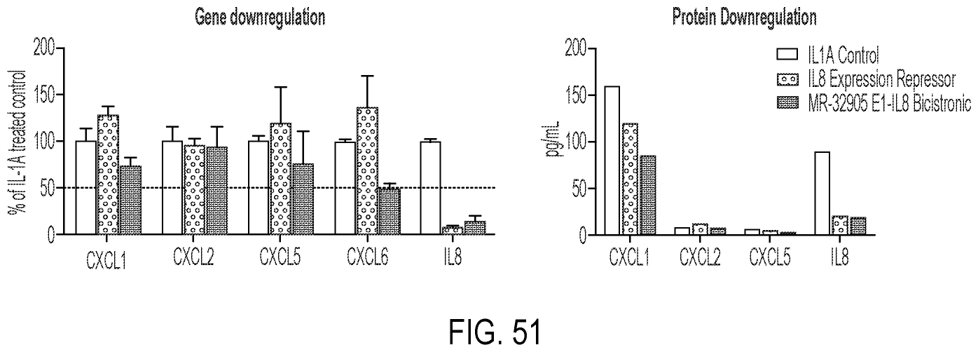

These results show targeting of both the E1 cRE and IL8 promoter region using the bicistronic mRNA construct (MR-32905; SEQ ID NO: (300)) can be used to decrease CXCL1, CXCL2, CXCL3, and IL8 expression in HSAEpC cells, and that expression is decreased at 48 hours post-treatment (FIG. 51).

### Example 35: CXCL1-8 Expression Downregulated in Bronchial Smooth Muscle Cells (Asthma)

This example describes an experiment demonstrating decreased expression of CXCL1, CXCL2, CXCL3 and IL8 in Bronchial smooth muscle cells (HBSMC) (Promocell C-12561 lot #397Z019.4), from a patient diagnosed with asthma, after treatment with an E11-IL8 bicistronic expression repressor.

HBSMC cells were transfected with an E1-IL8 targeting bicistronic expression repressor (MR-32905) or an IL8 promoter targeting expression repressor (MR-32712) added to the media at a final concentration of 1 g/ml SSOP lipid mix.

After completion of 48 hr incubation, IL1-alpha (Life Technologies Cat #PCH0015) was added at ng/ml final concentration and incubated for 1 hour.

After 1 hr incubation with IL1-alpha, RNA was isolated using the Macherey-Nagel Inc RNA extraction kit (cat #740466.4) following the Manufacture's protocol. RNA samples were retrotranscribed to cDNA using LunaScript RT SuperMix Kit (New England Biolabs) and analyzed by quantitative PCR using individual specific Taqman primer/probe sets with the Taqman Fast Advanced Master Mix (Thermo Scientific).

Gene expression was quantified relative to the human GAPDH reference gene using the ΔΔCt method. Changes in gene expression were further quantified by measuring the fold increase in gene expression after IL1 alpha treatment directly compared to the levels of gene expression in the untreated cells.

Protein levels were determined using the Thermo Fisher Luminex FlexMAP 3D Multiplexing Microplate reader. Supernatant was collected after 1 hr incubation with IL1-alpha and cytokines were measured using the ProcartaPlex 7 plex kit (cat #PPX-07-MXT2AW2.

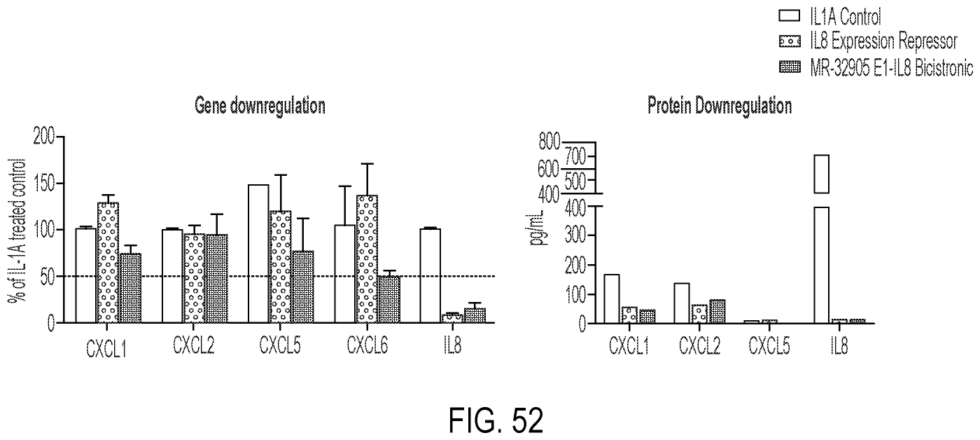

The results show targeting both the E1 cRE and IL8 promoter region using the bicistronic mRNA construct can be used to decrease CXCL1, CXCL2, and IL8 expression in HBSMC cells, and that expression is decreased at 48 hours post-treatment (FIG. 52).

### Example 36: CXCL1-8 Expression Downregulated in Primary Lung Fibroblast Cells

This example describes an experiment demonstrating decreased expression of CXCL1, CXCL2, CXCL3 and IL8 in human primary lung fibroblast cells (HPF) (Promocell C-12360 lot #474Z024.2

HPF cells were transfected with MR-32905 or MR-32712 added to the media at a final concentration of 1 g/ml SSOP lipid mix.

After completion of the 48 hr incubation, IL1-alpha (Life Technologies Cat #PCH0015) was added at 10 ng/ml final concentration and incubated for 1 hour.

After 1 hr incubation with IL1-alpha, RNA was isolated using the Macherey-Nagel Inc RNA extraction kit (cat #740466.4) following the manufacturer's protocol. RNA samples were retrotranscribed to cDNA using LunaScript RT SuperMix Kit (New England Biolabs) and analyzed by quantitative PCR using individual specific Taqman primer/probe sets with the Taqman Fast Advanced Master Mix (Thermo Scientific).

Gene expression was quantified relative to the human GAPDH reference gene using the ΔΔCt method. Changes in gene expression were further quantified by measuring the fold increase in gene expression after IL1 alpha treatment directly compared to the levels of gene expression in the untreated cells.

Protein levels were determined using the Thermo Fisher Luminex FlexMAP 3D Multiplexing Microplate reader. Supernatant was collected after 1 hr incubation with IL1-Alpha and cytokines were measured using the ProcartaPlex 7 plex kit (cat #PPX-07-MXT2AW2.

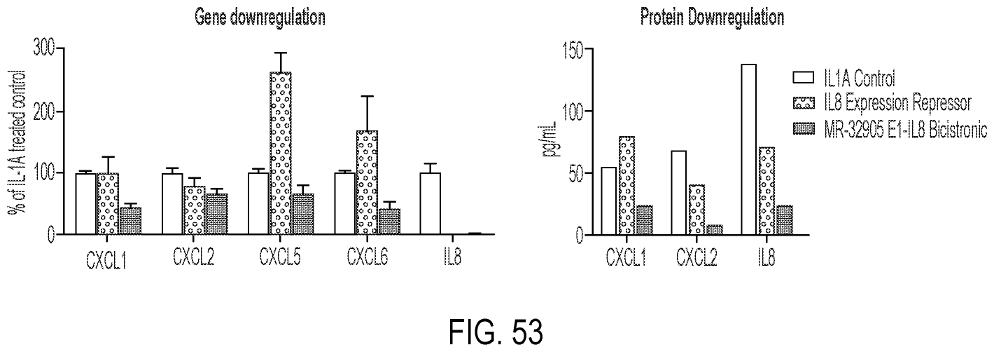

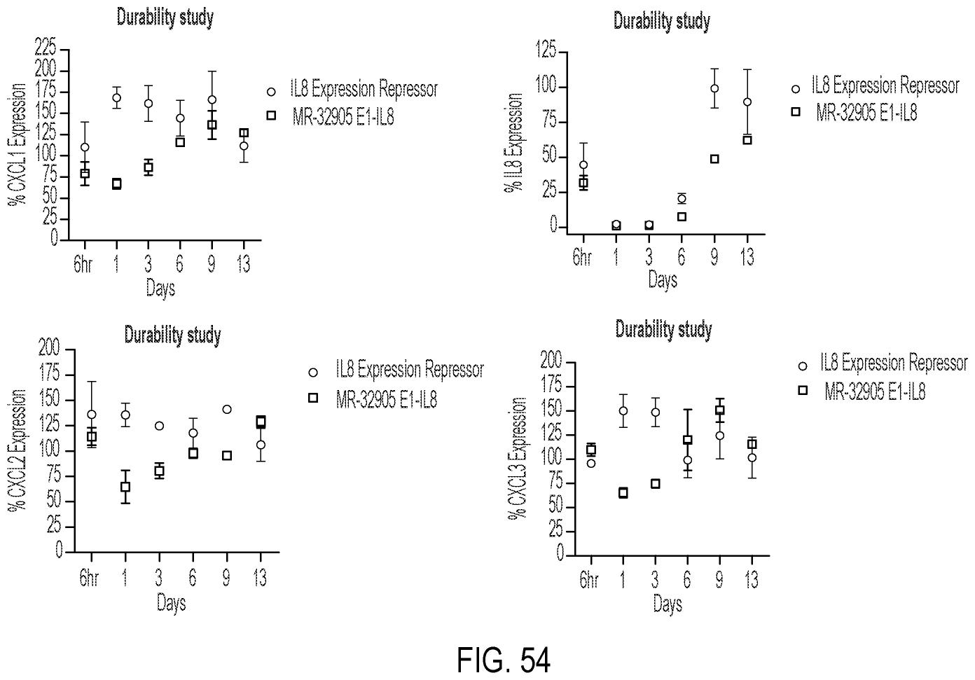

The results show targeting both the E1 cRE and IL8 promoter region using the bicistronic mRNA construct can be used to decrease CXCL1, CXCL2, and IL8 expression in HPF cells, and that expression is decreased at 48 hours post-treatment (FIG. 53).

Downregulation of genes in the CXCL locus was determined in a variety of cell types using the assays of Example 36 described herein. A summary of the downregulation observed is provided below in Tables 37 and 38.

TABLE 37
Downregulation of IL-8 in response to
treatment with MR-32712 or MR-32905

% IL8 Downregulation

| Primary human cell type | MR-32712 | MR-32905 |
|---|---|---|
| Bronchial smooth muscle cells | 98.5 ± 1.40 | 98.04 ± 0.97 |
| (Asthma patients) | | |
| Small airway epithelial cells | 91.31 ± 0.61 | 94.56 ± 2.63 |
| (COPD patients) | | |
| Macrophage* | 86.93 ± 3.35 | 50.26 ± 4.57 |
| Lung fibroblasts (Asthma patients) | 99.35 ± 0.19 | 99.17 ± 0.28 |
| Pulmonary artery endothelial cells | 99.31 ± 0.00 | 93.2 ± 0.96 |

Data generated with surrogate LNP (SSOP) unless otherwise noted.
*SM-102 (9-Heptadecanyl 8-{(2-hydroxyethyl)[6-oxo-6-(undecyloxy)hexyl]amino}octanoate) LNPs were used

TABLE 38
Downregulation of IL-8. CXCL1, CXCL2, CXCL5, and
CXCL6 in response to treatment with MR-32905

| | % Downregulation | | | | |
|---|---|---|---|---|---|
| Neutrophilic Asthma | IL-8 | CXCL1 | CXCL2 | CXCL5 | CXCL6 |
| Bronchial smooth muscle cells | 98.0 ± 1.0 | 28.5 ± 1.0 | 3.3 ± 5.2 | 41 ± 21 | ND |
| (asthma patients) | | | | | |
| Small airway epithelial cells | 94.6 ± 2.6 | 25.8 ± 8.4 | 6.2 ± 21.9 | 23.6 ± 33.9 | 50 ± 4.9 |
| (COPD patients) | | | | | |
| Macrophage* | 50.3 ± 4.6 | 42.5 ± 4.3 | 0 | 81.5 ± 8.5 | 47.9 ± 1.5 |
| Lung fibroblasts (asthma patient) | 99.2 ± 0.3 | 53.7 ± 14.9 | 45.7 ± 6.4 | 77.3 ± 3.7 | 74 ± 3.6 |
| Pulmonary artery endothelial cells | 93.2 ± 1.0 | 7.3 ± 10.4 | 0 | 0 | 64 ± 17.3 |

Data generated with surrogate LNP (SSOP) unless otherwise noted.
*SM-102 LNP were used

Overall, the results of Table 38 indicate that MR-32905 downregulates cytokines in primary cell types relevant to neutrophilic asthma

### Example 37: Durability of IL8 Downregulation Demonstrated with Bicistronic Expression Repressor

This example describes an experiment demonstrating decreased expression of IL8 in IMR-90 Cells (ATCCR CCL-186).

IMR-90 cells were cultured and plated in EMEM (ATCC-30-2003). Complete media was made with 10% FBS (VWR cat #97068-085).

IMR-90 cells were transfected with MR-32905 added to the media at a final concentration of 0.25 μg/ml SSOP lipid mix.

After completion of each specified time point, IL1-alpha (Life Technologies Cat #PCH0015) was added at 10 ng/ml final concentration and incubated for 1 hour.

After 1 hr incubation with IL1-alpha, RNA was isolated using the Machercy-Nagel Inc RNA extraction kit (cat #740466.4) following the manufacturer's protocol. RNA samples were retrotranscribed to cDNA using LunaScript RT SuperMix Kit (New England Biolabs) and analyzed by quantitative PCR using individual specific Taqman primer/probe sets with the Taqman Fast Advanced Master Mix (Thermo Scientific).

Gene expression was quantified relative to the human ABL1 reference gene using the ΔΔCt method. Changes in gene expression were further quantified by measuring the fold increase in gene expression after IL1 alpha treatment directly compared to the levels of gene expression in the untreated cells.

The results show targeting both the E1 enhancer region and IL8 promoter region using bicistronic mRNA can be used to decrease CXCL1, CXCL2, CXCL3, and IL8 expression in IMR90 cells over time (FIG. 53).

### Example 38: Decrease in Neutrophil Migration Greatest with Combination of E1-Targeting & IL8-Targeting Expression Repressor

This example describes an experiment demonstrating decreased neutrophil migration when neutrophils were exposed to cell supernatant from IMR90 cells containing various levels of chemokines.

IMR-90 cells (ATCC® CCL-186) were cultured and plated in EMEM (ATCC-30-2003) at 100k cells/well in 6 well plates. Complete media was made with 10% FBS (VWR cat #97068-085).

IMR-90 cells were transfected with MR-32105, MR-32715, MR-32905, or MR-32712+MR-32105 added to the media at a final concentration range of 1 μg/ml to 0.0008 μg/ml SSOP lipid mix.

Cells were incubated for 24 hrs with media containing LNPs. After 24 hrs point, IL1-alpha (Life Technologies Cat #PCH0015) was added at 10 ng/ml final concentration and incubated for 32 hours.

After incubation with IL1-alpha, the cell supernatant was collected and shipped to an external CRO (Charles River Labs) to conduct a neutrophil migration assay using ILIA treated supernatant as a control to which the other test groups were compared.

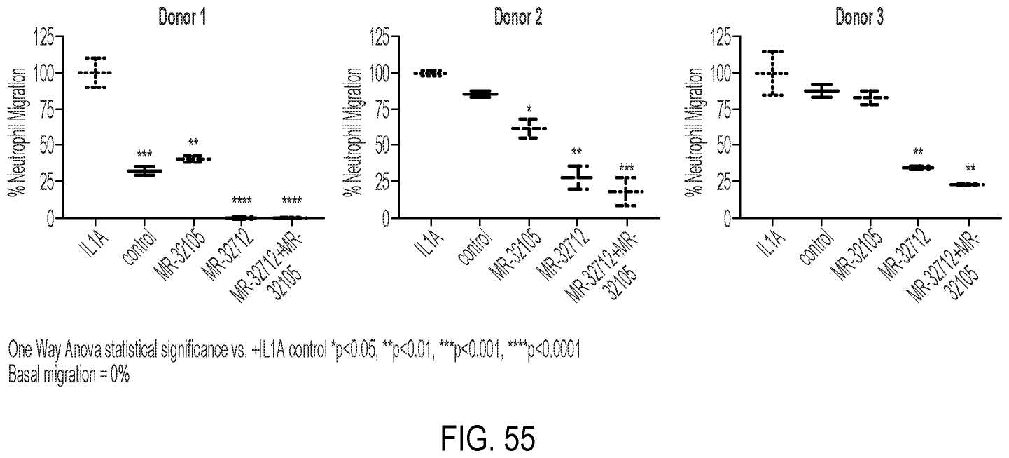

The results show that targeting both the E1 cRE and the IL8 promoter region demonstrated a more robust decrease in neutrophil migration (FIG. 55).

### Example 39: Substantial Reduction in Neutrophil Migration Shown with Bicistronic Expression Repressors

This example describes an experiment demonstrating decreased neutrophil migration when neutrophils were exposed to cell supernatant from IMR90 cells containing various levels of chemokines.

IMR-90 cells (ATCC® CCL-186) were cultured and plated in EMEM (ATCC-30-2003) at 100k cells/well in 6 well plates. Complete media was made with 10% FBS (VWR cat #97068-085).

IMR-90 cells were transfected with MR-32715 or MR-32905 added to the media at a final concentration range of 1 μg/ml to 0.0008 μg/ml SSOP lipid mix.

Cells were incubated for 24 hrs with media containing LNPs. After 24 hrs point, IL1-alpha (Life Technologies Cat #PCH0015) was added at 10 ng/ml final concentration and incubated for 32 hours.

After incubation with IL1-alpha, the cell supernatant was collected used to conduct a neutrophil migration assay using IL1A treated supernatant as a control to which the other test groups were compared.

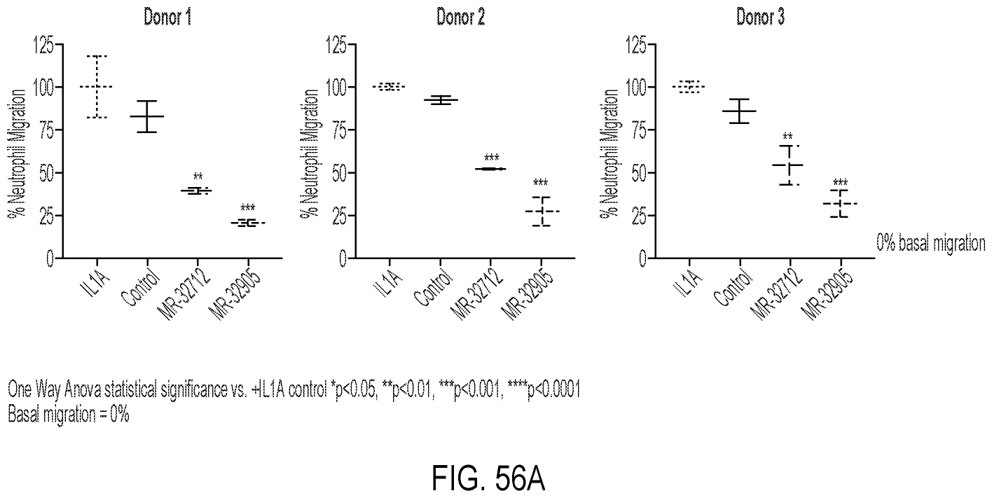

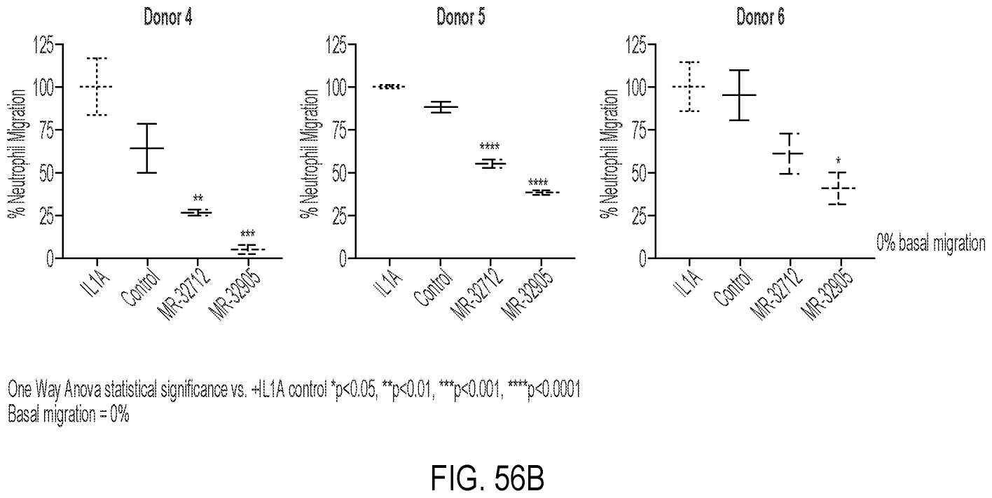

The results show that targeting both the E1 cRE and the IL8 promoter region using a bicistronic mRNA demonstrated a more robust decrease in neutrophil migration compared to IL8 promoter targeting mRNA alone (FIGS. 56A-56B).

### Example 40: Identification of Murine Homologue to Human E1

This example describes the identification of a murine genomic homologue to the human “E1” cRE targeted by an expression repressor described herein.

Murine E1 homologue was identified via P65 binding and sequence homology within the CXCL1-5, 15 murine IGD via analysis of P65-ChIP-seq after treatment in murine IL1A stimulation, in 3t3 fibroblast cells.

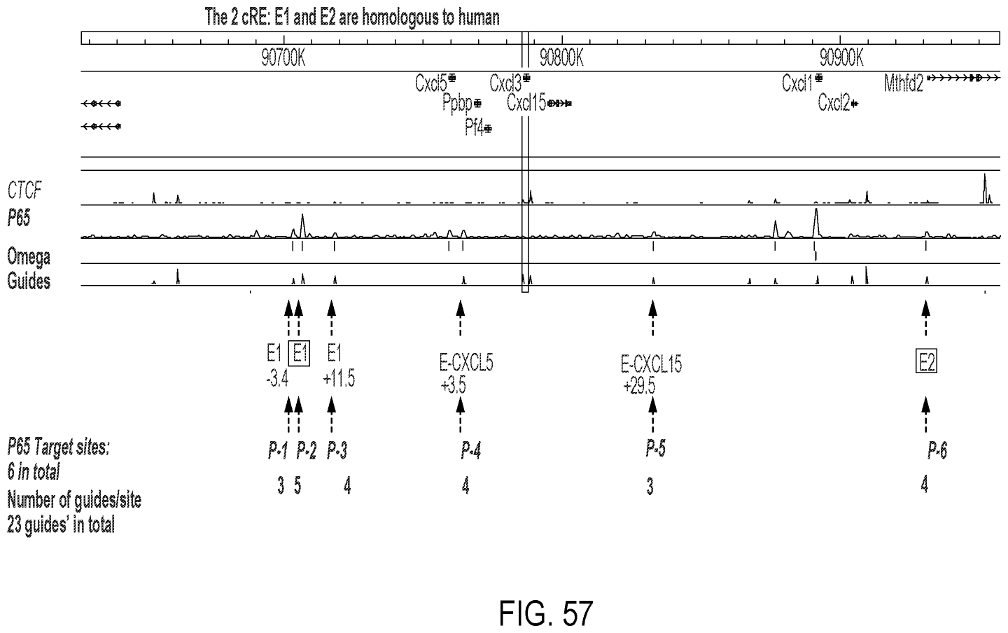

6 different P65 peak loci were identified within murine CXCL1-5, 15 IGD, with some sharing homologous sequence. Motivated assessment of each homologue was then performed to identify functional equivalence (see FIG. 57).

### Example 41: Screen for Functional Equivalence of Potential Murine E1 Homologues

This experiment describes the identification of a functional murine homologue cRE to hg E1. In particular, a screen was performed on candidate sites using dCas9-KRAB and targeted guides (Tables 32 and 33).

3t3 cells (ATCC: CRL-1658™) were cultured and plated in EMEM (ATCC-30-2003) at 5k cells/well in 96 well plates were seeded, and cells were allowed to be seeded for 24 hours, then transfected with guides and dCas9 KRAB at a final concentration of 0.5 μg/ml of SSOP lipid mix. Cells were incubated with LNPs containing guides (sgRNA+dcas9-KRAB) for 48 hours post-transfection, then IL1-A is added to 5 ng/ml final concentration and incubated for 2 hours. After 2 IL1-A stimulation, the cells supernatant was collected for ELISA and the cell pellets were collected to isolate RNA using the Macherey-Nagel Inc RNA extraction kit (cat #740466.4) following the manufacturer's protocol. RNA samples were retrotranscribed to cDNA using LunaScript RT SuperMix Kit (New England Biolabs) and analyzed by quantitative PCR using individual specific Taqman primer/probe sets with the Taqman Fast Advanced Master Mix (Thermo Scientific).

TABLE 32
Exemplary sequence or target sequences of gRNA
spacers

| Name | Sequence | SEQ ID NO. |
|---|---|---|
| GD-33448 | GTTAGCCCTAAATCGACCCT | 341 |
| GD-33449 | GGTGTGCAGTTAGCACAACT | 342 |
| GD-33450 | GAATTGCAGACATCAGTCTG | 343 |
| GD-33451 | AGGGGTCACCAAGAAGAGTG | 344 |
| GD-33452 | GGTTTCTCATTAGCAATCAG | 345 |
| GD-33453 | TCCTTGGGAAATTCCACTTG | 346 |
| GD-33454 | GTCATAACCCAGGAGTCACG | 347 |
| GD-33455 | GTTTCTGGGCTAGAGAACTC | 348 |
| GD-33456 | AATGGAACAAGTCAGCATGA | 349 |
| GD-33457 | CTGGCAAGGGTGAGACAGAG | 350 |
| GD-33458 | CATGGTAAACAACAGTACAC | 351 |
| GD-33459 | GTTGTGGATATATCCAGGCA | 352 |
| GD-33460 | TACGCATGAATGAGATCACT | 353 |
| GD-33461 | TTGTTTTTAATGCTTGCATG | 354 |
| GD-33462 | TCATTCCGTGACATCCAGGA | 355 |
| GD-33463 | CCTCCGTGACTAAGCCAAGG | 356 |
| GD-33464 | CCCCTACTCTCAGCCTGCAT | 357 |
| GD-33465 | ACTCCCCCTCTGTTTTACAC | 358 |
| GD-33466 | CTGAGTTTGTGTGGAGTATG | 359 |
| GD-33467 | TTTAGACTACTGTCTGACTG | 360 |
| GD-33468 | AGCTGTGGCAGTGATGCGAG | 361 |
| GD-33469 | AACAGGAAATATTCCTCCCA | 362 |
| GD-33470 | AGGATCTTCTGGAAGTGTCA | 363 |

TABLE 33
Exemplary guide sequences

| | | SEQ ID | | Genomic |
|---|---|---|---|---|
| Guide | Sequence | NO | Strand | Coordinate |
| GD-33448 | GTTAGCCCTAAATCGACCCTGTTT | 364 | − | GRCm38: |
| | TAGAGCTAGAAATAGCAAGTTAAA | | | chr5: |
| | ATAAGGCTAGTCCGTTATCAACTT | | | 90702981-90703003 |
| | GAAAAAGTGGCACCGAGTCGGTGC | | | |
| | TTTT | | | |
| GD-33449 | GGTGTGCAGTTAGCACAACTGTTT | 365 | + | GRCm38: |
| | TAGAGCTAGAAATAGCAAGTTAAA | | | chr5: |
| | ATAAGGCTAGTCCGTTATCAACTT | | | 90703037-90703059 |
| | GAAAAAGTGGCACCGAGTCGGTGC | | | |
| | TTTT | | | |
| GD-33450 | GAATTGCAGACATCAGTCTGGTTT | 366 | − | GRCm38: |
| | TAGAGCTAGAAATAGCAAGTTAAA | | | chr5: |
| | ATAAGGCTAGTCCGTTATCAACTT | | | 90703085-90703107 |
| | GAAAAAGTGGCACCGAGTCGGTGC | | | |
| | TTTT | | | |
| GD-33451 | AGGGGTCACCAAGAAGAGTGGTTT | 367 | − | GRCm38: |
| | TAGAGCTAGAAATAGCAAGTTAAA | | | chr5: |
| | ATAAGGCTAGTCCGTTATCAACTT | | | 90706313-90706335 |
| | GAAAAAGTGGCACCGAGTCGGTGC | | | |
| | TTTT | | | |
| GD-33452 | GGTTTCTCATTAGCAATCAGGTTT | 368 | − | GRCm38: |
| | TAGAGCTAGAAATAGCAAGTTAAA | | | chr5: |
| | ATAAGGCTAGTCCGTTATCAACTT | | | 31-90706353 |
| | GAAAAAGTGGCACCGAGTCGGTGC | | | 907063 |
| | TTTT | | | |
| GD-33453 | TCCTTGGGAAATTCCACTTGGTTT | 369 | + | GRCm38: |
| | TAGAGCTAGAAATAGCAAGTTAAA | | | chr5: |
| | ATAAGGCTAGTCCGTTATCAACTT | | | 90706470-90706492 |
| | GAAAAAGTGGCACCGAGTCGGTGC | | | |
| | TTTT | | | |
| GD-33454 | GTCATAACCCAGGAGTCACGGTTT | 370 | − | GRCm38: |
| | TAGAGCTAGAAATAGCAAGTTAAA | | | chr5: |
| | ATAAGGCTAGTCCGTTATCAACTT | | | 90706505-90706527 |
| | GAAAAAGTGGCACCGAGTCGGTGC | | | |
| | TTTT | | | |
| GD-33455 | GTTTCTGGGCTAGAGAACTCGTTT | 371 | + | GRCm38: |
| | TAGAGCTAGAAATAGCAAGTTAAA | | | chr5: |
| | ATAAGGCTAGTCCGTTATCAACTT | | | 29070660-90706642 |
| | GAAAAAGTGGCACCGAGTCGGTGC | | | |
| | TTTT | | | |
| GD-33456 | AATGGAACAAGTCAGCATGAGTTT | 372 | + | GRCm38: |
| | TAGAGCTAGAAATAGCAAGTTAAA | | | chr5: |
| | ATAAGGCTAGTCCGTTATCAACTT | | | 39071795-90717957 |
| | GAAAAAGTGGCACCGAGTCGGTGC | | | |
| | TTTT | | | |
| GD-33457 | ctggcaagggtgagacagagGTTT | 373 | + | GRCm38: |
| | TAGAGCTAGAAATAGCAAGTTAAA | | | chr5: |
| | ATAAGGCTAGTCCGTTATCAACTT | | | 90718084-90718106 |
| | GAAAAAGTGGCACCGAGTCGGTGC | | | |
| | TTTT | | | |
| GD-33458 | catggtaaacaacagtacacGTTT | 374 | − | GRCm38: |
| | TAGAGCTAGAAATAGCAAGTTAAA | | | chr5: |
| | ATAAGGCTAGTCCGTTATCAACTT | | | 90718111-90718133 |
| | GAAAAAGTGGCACCGAGTCGGTGC | | | |
| | TTTT | | | |
| GD-33459 | gttgtggatatatccaggcaGTTT | 375 | − | GRCm38: |
| | TAGAGCTAGAAATAGCAAGTTAAA | | | chr5: |
| | ATAAGGCTAGTCCGTTATCAACTT | | | 90718179-90718201 |
| | GAAAAAGTGGCACCGAGTCGGTGC | | | |
| | TTTT | | | |
| GD-33460 | TACGCATGAATGAGATCACTGTTT | 376 | + | GRCm38: |
| | TAGAGCTAGAAATAGCAAGTTAAA | | | chr5: |
| | ATAAGGCTAGTCCGTTATCAACTT | | | 90764062-90764084 |
| | GAAAAAGTGGCACCGAGTCGGTGC | | | |
| | TTTT | | | |
| GD-33461 | TTGTTTTTAATGCTTGCATGGTTT | 377 | + | GRCm38: |
| | TAGAGCTAGAAATAGCAAGTTAAA | | | chr5: |
| | ATAAGGCTAGTCCGTTATCAACTT | | | 90764268-90764290 |
| | GAAAAAGTGGCACCGAGTCGGTGC | | | |
| | TTTT | | | |
| GD-33462 | TCATTCCGTGACATCCAGGAGTTT | 378 | − | GRCm38: |
| | TAGAGCTAGAAATAGCAAGTTAAA | | | chr5: |
| | ATAAGGCTAGTCCGTTATCAACTT | | | 90764336-90764358 |
| | GAAAAAGTGGCACCGAGTCGGTGC | | | |
| | TTTT | | | |
| GD-33463 | CCTCCGTGACTAAGCCAAGGGTTT | 379 | + | GRCm38: |
| | TAGAGCTAGAAATAGCAAGTTAAA | | | chr5: |
| | ATAAGGCTAGTCCGTTATCAACTT | | | 90764401-90764423 |
| | GAAAAAGTGGCACCGAGTCGGTGC | | | |
| | TTTT | | | |
| GD-33464 | CCCCTACTCTCAGCCTGCATGTTT | 380 | − | GRCm38: |
| | TAGAGCTAGAAATAGCAAGTTAAA | | | chr5: |
| | ATAAGGCTAGTCCGTTATCAACTT | | | 90832483-90832505 |
| | GAAAAAGTGGCACCGAGTCGGTGC | | | |
| | TTTT | | | |
| GD-33465 | ACTCCCCCTCTGTTTTACACGTTT | 381 | + | GRCm38: |
| | TAGAGCTAGAAATAGCAAGTTAAA | | | chr5: |
| | ATAAGGCTAGTCCGTTATCAACTT | | | 90832526-90832548 |
| | GAAAAAGTGGCACCGAGTCGGTGC | | | |
| | TTTT | | | |
| GD-33466 | CTGAGTTTGTGTGGAGTATGGTTT | 382 | + | GRCm38: |
| | TAGAGCTAGAAATAGCAAGTTAAA | | | chr5: |
| | ATAAGGCTAGTCCGTTATCAACTT | | | 90832598-90832620 |
| | GAAAAAGTGGCACCGAGTCGGTGC | | | |
| | TTTT | | | |
| GD-33467 | tttagactactgtctgactgGTTT | 383 | − | GRCm38: |
| | TAGAGCTAGAAATAGCAAGTTAAA | | | chr5: |
| | ATAAGGCTAGTCCGTTATCAACTT | | | 90930741-90930763 |
| | GAAAAAGTGGCACCGAGTCGGTGC | | | |
| | TTTT | | | |
| GD-33468 | agctgtggcagtgatgcgagGTTT | 384 | − | GRCm38: |
| | TAGAGCTAGAAATAGCAAGTTAAA | | | chr5: |
| | ATAAGGCTAGTCCGTTATCAACTT | | | 90930773-90930795 |
| | GAAAAAGTGGCACCGAGTCGGTGC | | | |
| | TTTT | | | |
| GD-33469 | aacaggaaatattcctcccaGTTT | 385 | − | GRCm38: |
| | TAGAGCTAGAAATAGCAAGTTAAA | | | chr5: |
| | ATAAGGCTAGTCCGTTATCAACTT | | | 90930799-90930821 |
| | GAAAAAGTGGCACCGAGTCGGTGC | | | |
| | TTTT | | | |
| GD-33470 | AGGATCTTCTGGAAGTGTCAGTTT | 386 | + | GRCm38: |
| | TAGAGCTAGAAATAGCAAGTTAAA | | | chr5: |
| | ATAAGGCTAGTCCGTTATCAACTT | | | 90930887-90930909 |
| | GAAAAAGTGGCACCGAGTCGGTGC | | | |
| | TTTT | | | |

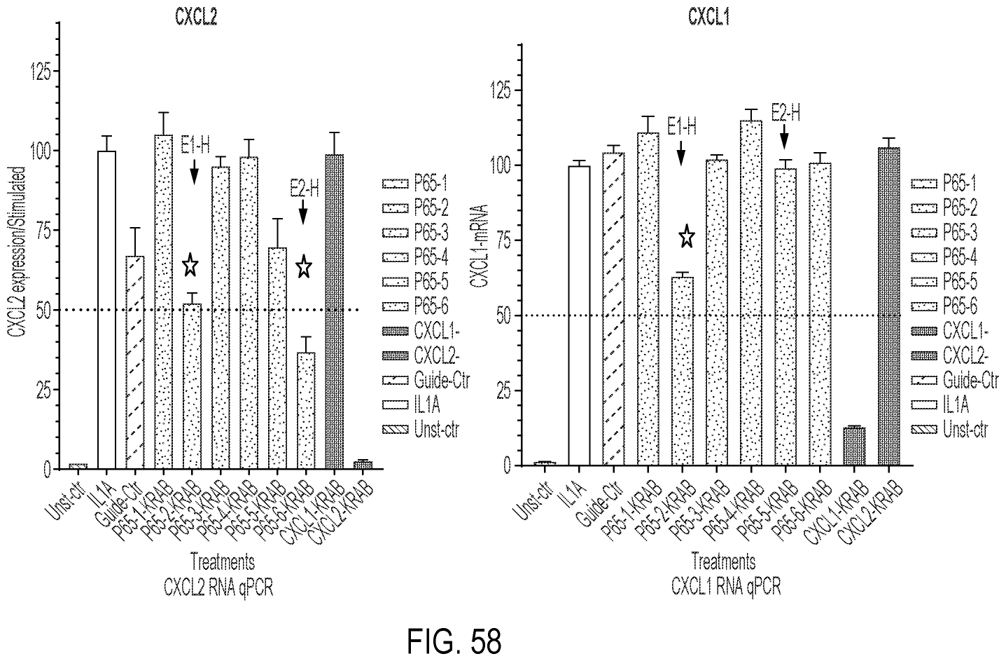

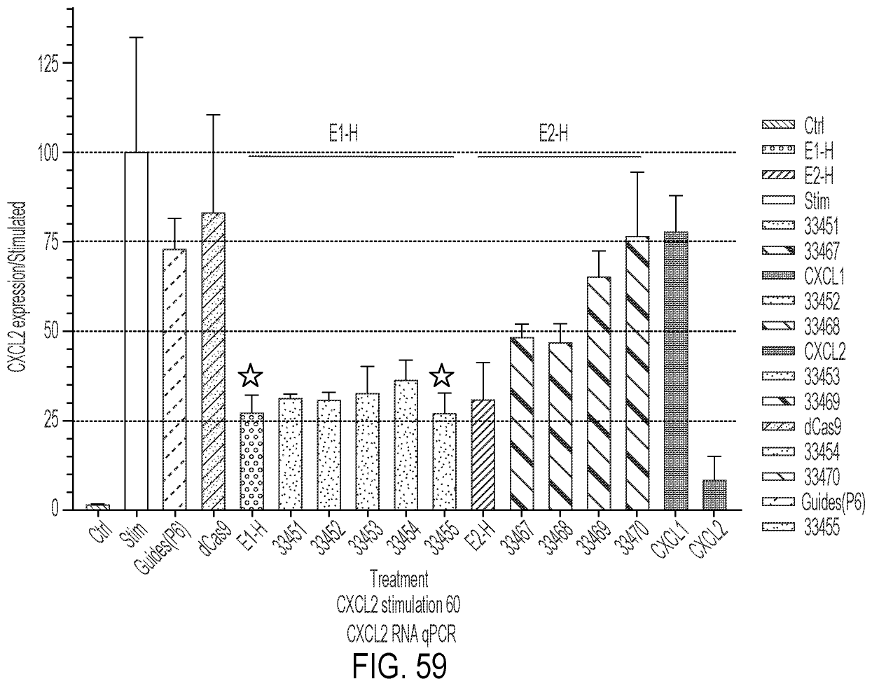

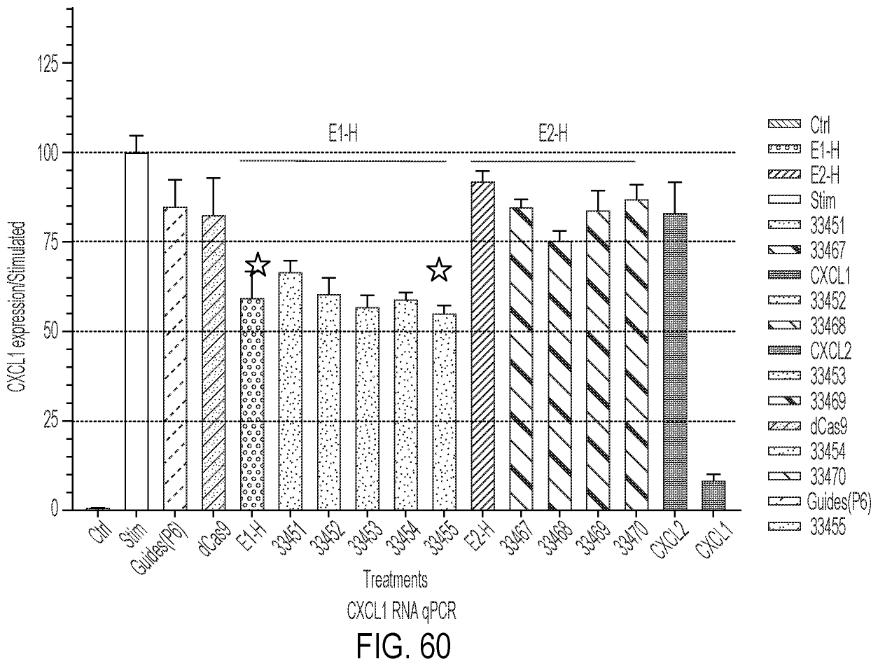

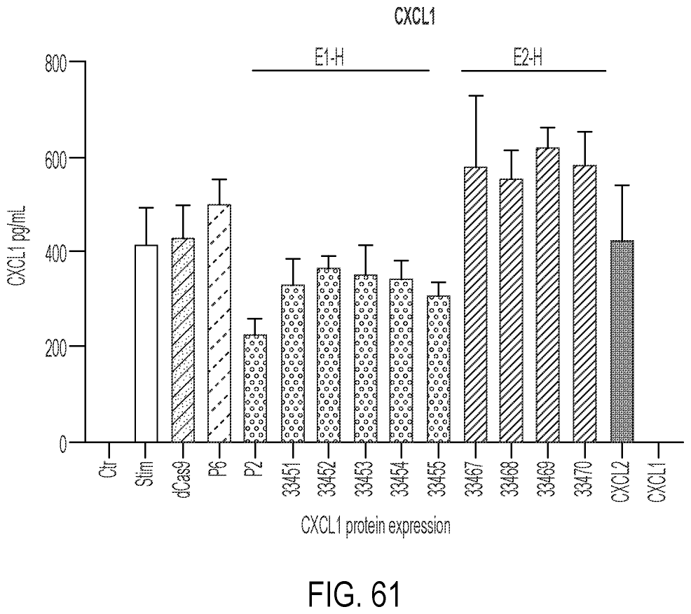

Two notable P65 peak regions identified, one with sequence homology to human E1, presenting functional consequence. CXCL1 is downregulated 60% only when targeting P65 peak ‘P65-P2’ while CXCL2 is downregulated to 50% when targeting ‘P65-P2’ and to 40% when targeting ‘P65-P6.’ The experiment identifies one lead guide GD-33455 targeting P65-P2 peak that downregulated CXCL1 & CXCL2 to 60% and 50% transcriptional activity. This site exhibits sequence homology with human locus targeted by MR-32015 (FIGS. 58-61).

### Example 42: CXCL1 and CXCL2 Downregulation in Mouse Homologues

3T3 cells were plated at 5k cells per well in a flat bottom cell culture treated plate in 100 μl of media (DMEM Gibco Cat #11995-065 10% FBS VWR cat #97068-085). After 24 hours adhering to the plate, SSOP LNPs were used to formulate MR-33720-MR-33723 (SEQ ID NOs: 313, 320, 327, and 334, respectively; Tables 34-36). Cells were transfected at 0.2 μg/mL or 1 μg/mL for 48 hours. At 48 hours, cells were washed and stimulated by incubating the transfected cells with 5 ng/mL of mouse IL1A. The supernatants were removed for ELISA and the plate was processed for qPCR. RNA was isolated using the Macherey-Nagel Inc RNA extraction kit (cat #740466.4) following the manufacturer's protocol. RNA samples were retrotranscribed to cDNA using LunaScript RT SuperMix Kit (New England Biolabs) and analyzed by quantitative PCR using individual specific Taqman primer/probe sets with the Taqman Fast Advanced Master Mix (Thermo Scientific).

Gene expression was quantified relative to the mouse HPRT reference gene using the ΔΔCt method. Changes in gene expression were further quantified by measuring the fold increase in gene expression after TNF alpha treatment directly compared to the levels of gene expression in the untreated cells.

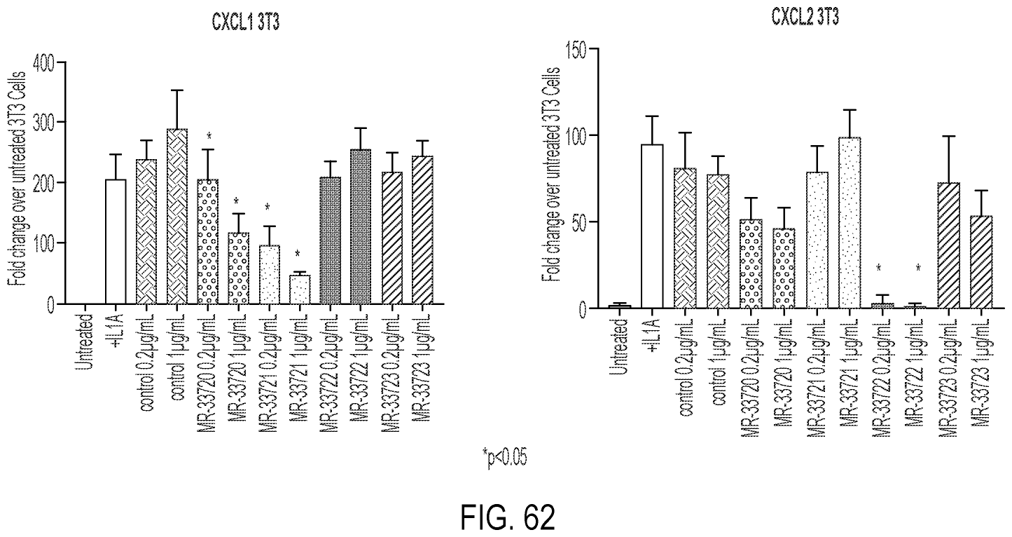

MR-33720 and MR-33721 showed robust decrease in downregulating of CXCL1 at 1 μg/mL concentration. MR-33722 showed the highest decrease in CXCL2 gene expression. (FIG. 62). Without wishing to be bound by theory, the genomic location of murine CXCL1 and CXCL2 in the CXCL locus of the murine genome are analogous to the genomic location of human IL-8 in the CXCL locus of the human genome. In some embodiments, targeting murine CXCL1 or CXCL2, or a cis-acting regulatory element of CXCL1 or CXCL2, as described herein, is analogous to targeting human IL-8, or a cis-acting regulatory element of IL-8. In certain embodiments, the results from targeting murine CXCL1 or CXCL2, or a cis-acting regulatory element of CXCL1 or CXCL2, can be indicative of the effects of targeting human IL-8, or a cis-acting regulatory element of human IL-8. Consequently, the present disclosure provides certain expression repressors that target murine CXCL1 or CXCL2, which can be used as mouse surrogates for expression repressors that target human IL-8.

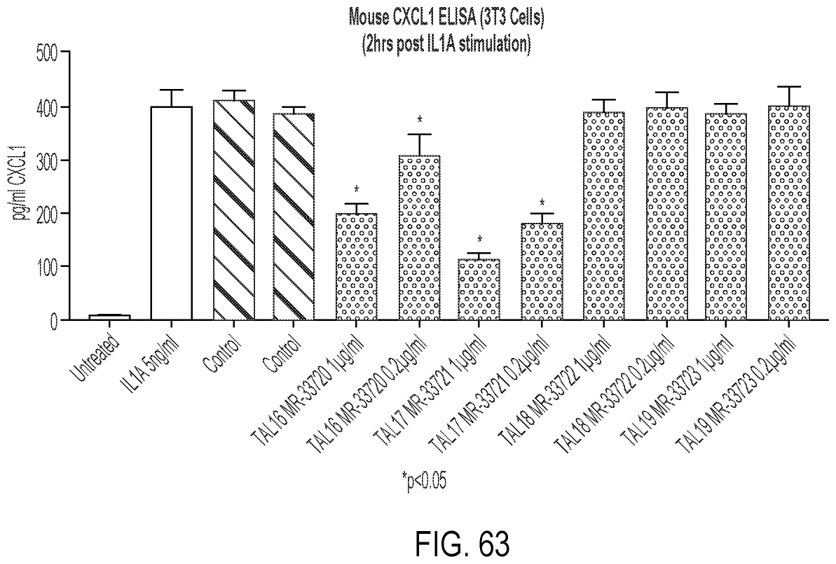

The supernatant was harvested as described above and mouse CXCL1 ELISA (Abeam catalog ab216951) was used to quantify the protein levels present in the supernatant. MR-33720 and MR-33721 showed the most significant decrease CXCL1 protein (FIG. 63).

TABLE 34
Exemplary mouse TAL expression repressors.

| SEQ | | |
|---|---|---|
| ID | | |
| NO | Description | Sequence |
| 312 | Mouse CXCL | MAPKKKRKVGGSGGSHMVDLRTLGYSQQQQEKIKPKVRSTVAQHHEAL |
| | TAL16-KRAB | VGHGFTHAHIVALSQHPAALGTVAVKYQDMIAALPEATHEAIVGVGKRG |
| | Protein | AGARALEALLTVAGELRGPPLOLDTGOLLKIAKRGGVTAVEAVHAWRNAL |
| | sequence | TGAPLNLTPEQVVAIASNNGGKQALETVQRLLPVLCQAHGLTPEQVVAIA |
| | (MR-33720) | SNGGGKQALETVQRLLPVLCQAHGLTPEQVVAIASHDGGKQALETVQRLL |
| | | PVLCQAHGLTPEQVVAIASNGGGKQALETVQRLLPVLCQAHGLTPEQVV |
| | | AIASNIGGKQALETVQRLLPVLCQAHGLTPEQVVAIASNIGGKQALETVQR |
| | | LLPVLCQAHGLTPEQVVAIASNGGGKQALETVQRLLPVLCQAHGLTPEQV |
| | | VAIASHDGGKQALETVQRLLPVLCQAHGLTPEQVVAIASHDGGKQALETV |
| | | QRLLPVLCQAHGLTPEQVVAIASNGGGKQALETVQRLLPVLCQAHGLTPE |
| | | QVVAIASNGGGKQALETVQRLLPVLCQAHGLTPEQVVAIASNNGGKQAL |
| | | ETVQRLLPVLCQAHGLTPEQVVAIASRHGGKQALETVQRLLPVLCQAHGL |
| | | TPEQVVAIASRHGGKQALETVQRLLPVLCQAHGLTPEQVVAIASNIGGKQ |
| | | ALETVQRLLPVLCQAHGLTPEQVVAIASNNGGKQALETVQRLLPVLCQAH |
| | | GLTPEQVVAIASNGGGKQALETVQRLLPVLCQAHGLTPEQVVAIASNNG |
| | | GRPALESIVAQLSRPDPALAALTNDHLVALACLGGRPALDAVKKGLPHAP |
| | | ALIKRTNRRIPERTSHRVASGSGGGSGGARDDAKSLTAWSRTLVTFKDVFV |
| | | DFTREEWKLLDTAQQILYRNVMLENYKNLVSLGYQLTKPDVILRLEKGEEP |
| | | WLVEREIHQETHPDSETAFEIKSSVPSSGGKRPAATKKAGQAKKKKGSYPY |
| | | DVPDYA* |
| 319 | Mouse CXCL | MAPKKKRKVGGSGGSHMVDLRTLGYSQQQQEKIKPKVRSTVAQHHEAL |
| | TAL17-KRAB | VGHGFTHAHIVALSQHPAALGTVAVKYQDMIAALPEATHEAIVGVGKRG |
| | Protein | AGARALEALLTVAGELRGPPLQLDTGQLLKIAKRGGVTAVEAVHAWRNAL |
| | sequence | TGAPLNLTPEQVVAIASHDGGKQALETVQRLLPVLCQAHGLTPEQVVAIA |
| | (MR-33721) | SNGGGKQALETVQRLLPVLCQAHGLTPEQVVAIASNNGGKQALETVQRL |
| | | LPVLCQAHGLTPEQVVAIASNGGGKQALETVQRLLPVLCQAHGLTPEQVV |
| | | AIASNIGGKQALETVQRLLPVLCQAHGLTPEQVVAIASNNGGKQALETVQ |
| | | RLLPVLCQAHGLTPEQVVAIASNGGGKQALETVQRLLPVLCQAHGLTPEQ |
| | | VVAIASNIGGKQALETVQRLLPVLCQAHGLTPEQVVAIASHDGGKQALET |
| | | VQRLLPVLCQAHGLTPEQVVAIASNIGGKQALETVQRLLPVLCQAHGLTPE |
| | | QVVAIASHDGGKQALETVQRLLPVLCQAHGLTPEQVVAIASHDGGKQAL |
| | | ETVQRLLPVLCQAHGLTPEQVVAIASNIGGKQALETVQRLLPVLCQAHGLT |
| | | PEQVVAIASNIGGKQALETVQRLLPVLCQAHGLTPEQVVAIASNNGGKQA |
| | | LETVQRLLPVLCQAHGLTPEQVVAIASNGGGKQALETVQRLLPVLCQAHG |
| | | LTPEQVVAIASNNGGKQALETVQRLLPVLCQAHGLTPEQVVAIASRHGGR |
| | | PALESIVAQLSRPDPALAALTNDHLVALACLGGRPALDAVKKGLPHAPALI |
| | | KRTNRRIPERTSHRVASGSGGGSGGARDDAKSLTAWSRTLVTFKDVFVDF |
| | | TREEWKLLDTAQQILYRNVMLENYKNLVSLGYQLTKPDVILRLEKGEEPWL |
| | | VEREIHQETHPDSETAFEIKSSVPSSGGKRPAATKKAGQAKKKKGSYPYDV |
| | | PDYA* |
| 326 | Mouse CXCL | MAPKKKRKVGGSGGSHMVDLRTLGYSQQQQEKIKPKVRSTVAQHHEAL |
| | TAL18-KRAB | VGHGFTHAHIVALSQHPAALGTVAVKYQDMIAALPEATHEAIVGVGKRG |
| | Protein | AGARALEALLTVAGELRGPPLQLDTGQLLKIAKRGGVTAVEAVHAWRNAL |
| | sequence | TGAPLNLTPEQVVAIASNNGGKQALETVQRLLPVLCQAHGLTPEQVVAIA |
| | (MR-33722) | SNGGGKQALETVQRLLPVLCQAHGLTPEQVVAIASNGGGKQALETVQRL |
| | | LPVLCQAHGLTPEQVVAIASNIGGKQALETVQRLLPVLCQAHGLTPEQVV |
| | | AIASNNGGKQALETVQRLLPVLCQAHGLTPEQVVAIASRHGGKQALETVQ |
| | | RLLPVLCQAHGLTPEQVVAIASNGGGKQALETVQRLLPVLCQAHGLTPEQ |
| | | VVAIASHDGGKQALETVQRLLPVLCQAHGLTPEQVVAIASNGGGKQALET |
| | | VQRLLPVLCQAHGLTPEQVVAIASHDGGKQALETVQRLLPVLCQAHGLTP |
| | | EQVVAIASHDGGKQALETVQRLLPVLCQAHGLTPEQVVAIASNIGGKQAL |
| | | ETVQRLLPVLCQAHGLTPEQVVAIASHDGGKQALETVQRLLPVLCQAHGL |
| | | TPEQVVAIASNGGGKQALETVQRLLPVLCQAHGLTPEQVVAIASNNGGK |
| | | QALETVQRLLPVLCQAHGLTPEQVVAIASNIGGKQALETVQRLLPVLCQA |
| | | HGLTPEQVVAIASNGGGKQALETVQRLLPVLCQAHGLTPEQVVAIASNN |
| | | GGRPALESIVAQLSRPDPALAALTNDHLVALACLGGRPALDAVKKGLPHA |
| | | PALIKRTNRRIPERTSHRVASGSGGGSGGARDDAKSLTAWSRTLVTFKDVF |
| | | VDFTREEWKLLDTAQQILYRNVMLENYKNLVSLGYQLTKPDVILRLEKGEE |
| | | PWLVEREIHQETHPDSETAFEIKSSVPSSGGKRPAATKKAGQAKKKKGSYP |
| | | YDVPDYA* |
| 333 | Mouse CXCL | MAPKKKRKVGGSGGSHMVDLRTLGYSQQQQEKIKPKVRSTVAQHHEAL |
| | TAL 19-KRAB | VGHGFTHAHIVALSQHPAALGTVAVKYQDMIAALPEATHEAIVGVGKRG |
| | Protein | AGARALEALLTVAGELRGPPLQLDTGQLLKIAKRGGVTAVEAVHAWRNAL |
| | sequence | TGAPLNLTPEQVVAIASHDGGKQALETVQRLLPVLCQAHGLTPEQVVAIA |
| | (MR-33723) | SNIGGKQALETVQRLLPVLCQAHGLTPEQVVAIASNNGGKQALETVQRLL |
| | | PVLCQAHGLTPEQVVAIASRHGGKQALETVQRLLPVLCQAHGLTPEQVVA |
| | | IASRHGGKQALETVQRLLPVLCQAHGLTPEQVVAIASNIGGKQALETVQRL |
| | | LPVLCQAHGLTPEQVVAIASNIGGKQALETVQRLLPVLCQAHGLTPEQVV |
| | | AIASNGGGKQALETVQRLLPVLCQAHGLTPEQVVAIASNGGGKQALETV |
| | | QRLLPVLCQAHGLTPEQVVAIASNGGGKQALETVQRLLPVLCQAHGLTPE |
| | | QVVAIASHDGGKQALETVQRLLPVLCQAHGLTPEQVVAIASHDGGKQAL |
| | | ETVQRLLPVLCQAHGLTPEQVVAIASHDGGKQALETVQRLLPVLCQAHGL |
| | | TPEQVVAIASNGGGKQALETVQRLLPVLCQAHGLTPEQVVAIASNNGGK |
| | | QALETVQRLLPVLCQAHGLTPEQVVAIASRHGGKQALETVQRLLPVLCQA |
| | | HGLTPEQVVAIASNGGGKQALETVQRLLPVLCQAHGLTPEQVVAIASHDG |
| | | GRPALESIVAQLSRPDPALAALTNDHLVALACLGGRPALDAVKKGLPHAP |
| | | ALIKRTNRRIPERTSHRVASGSGGGSGGARDDAKSLTAWSRTLVTFKDVFV |
| | | DFTREEWKLLDTAQQILYRNVMLENYKNLVSLGYQLTKPDVILRLEKGEEP |
| | | WLVEREIHQETHPDSETAFEIKSSVPSSGGKRPAATKKAGQAKKKKGSYPY |
| | | DVPDYA* |

In the amino acid sequences of this table, the DBD is doubly underlined and the effector region is singly underlined.

TABLE 35
Exemplary nucleic acid sequences encoding TAL expression repressors

| SEQ ID NO | Description | Sequence |
|---|---|---|
| 313 | Mouse CXCL | GGGAAAUAAGAGAGAAAAGAAGAGUAAGAAGAAAUAUAAGAGCCA |
| | TAL16-KRAB | CCAUGGCCCCAAAGAAGAAGCGGAAAGUCGGAGGUUCAGGCGGCU |
| | mRNA sequence | CGCACAUGGUCGACCUGAGGACCCUCGGAUACUCGCAGCAGCAGC |
| | (MR-33720) | AGGAAAAGAUCAAGCCUAAGGUCCGCAGCACCGUGGCUCAGCAUC |
| | | AUGAGGCCCUUGUGGGACACGGGUUCACCCACGCCCAUAUUGUGG |
| | | CCCUGUCCCAACACCCGGCUGCACUGGGCACCGUGGCCGUGAAGU |
| | | ACCAGGACAUGAUCGCCGCCCUGCCCGAGGCGACCCACGAGGCCA |
| | | UCGUGGGAGUGGGAAAGCGCGGCGCCGGUGCCAGAGCACUGGAAG |
| | | CGCUGCUGACUGUGGCGGGAGAGCUGAGGGGACCUCCACUCCAAU |
| | | UGGAUACCGGGCAGCUCCUGAAGAUCGCCAAGCGCGGCGGAGUGA |
| | | CCGCCGUGGAAGCCGUGCACGCCUGGCGGAACGCGCUGACUGGGG |
| | | CCCCGCUGAACCUGACCCCAGAACAGGUUGUGGCUAUCGCCUCCA |
| | | ACAACGGCGGAAAACAGGCCUUGGAGACUGUGCAGCGGCUUCUGC |
| | | CGGUGCUUUGUCAAGCCCACGGCCUGACCCCGGAACAGGUCGUGG |
| | | CCAUCGCCUCAAACGGCGGCGGGAAGCAGGCUCUCGAAACCGUGC |
| | | AACGGCUCCUGCCCGUGCUGUGCCAAGCUCAUGGUCUGACACCUG |
| | | AACAAGUGGUGGCGAUUGCGUCCCACGACGGGGGCAAACAGGCCC |
| | | UCGAAACUGUCCAGCGGCUGCUGCCCGUGUUGUGCCAAGCACACG |
| | | GACUCACGCCCGAACAAGUCGUCGCGAUUGCAAGCAAUGGUGGUG |
| | | GAAAGCAGGCCCUGGAGACUGUCCAGAGACUCCUGCCCGUGCUGU |
| | | GCCAGGCUCACGGGCUGACCCCGGAACAAGUCGUGGCCAUUGCCA |
| | | GCAACAUCGGCGGCAAACAGGCUCUGGAGACUGUGCAGCGCCUGC |
| | | UGCCAGUGCUGUGUCAGGCGCACGGACUGACCCCCGAACAGGUCG |
| | | UGGCAAUCGCAUCAAACAUCGGAGGAAAACAGGCCUUGGAAACCG |
| | | UCCAGCGGCUGUUGCCCGUGCUUUGCCAAGCUCAUGGACUCACCC |
| | | CUGAGCAAGUCGUCGCGAUUGCCUCCAACGGUGGAGGAAAGCAAG |
| | | CCCUUGAGACUGUCCAGAGGCUGCUGCCGGUGUUAUGCCAGGCCC |
| | | ACGGUCUGACCCCAGAACAGGUGGUGGCCAUCGCCAGCCAUGACG |
| | | GUGGCAAACAAGCCCUCGAAACAGUGCAGAGACUGCUCCCGGUCC |
| | | UGUGCCAAGCCCACGGCCUCACCCCGGAGCAAGUGGUGGCUAUCG |
| | | CCUCGCACGAUGGAGGGAAGCAAGCGCUGGAAACCGUGCAGCGGU |
| | | UGCUGCCCGUCCUGUGUCAAGCGCACGGCCUGACUCCGGAACAGG |
| | | UAGUCGCCAUUGCGUCCAACGGCGGCGGGAAGCAGGCGCUCGAAA |
| | | CGGUGCAGCGGCUGCUGCCUGUGCUCUGCCAAGCACACGGACUUA |
| | | CCCCUGAACAAGUGGUCGCCAUCGCCUCCAAUGGUGGCGGAAAGC |
| | | AGGCCCUGGAAACUGUGCAACGCCUCCUGCCCGUGUUGUGCCAGG |
| | | CGCAUGGGCUCACUCCUGAACAGGUUGUCGCAAUCGCUUCCAACA |
| | | ACGGCGGGAAACAGGCACUGGAAACCGUGCAGCGGCUGUUGCCAG |
| | | UGUUGUGCCAAGCGCACGGCCUGACUCCUGAACAAGUGGUGGCCA |
| | | UUGCGUCGCGGCAUGGUGGCAAACAGGCCCUCGAAACUGUGCAAC |
| | | GGCUGCUGCCGGUGUUGUGUCAGGCGCACGGACUGACCCCGGAGC |
| | | AAGUCGUCGCUAUCGCGAGCAGACACGGCGGCAAGCAGGCCCUGG |
| | | AAACCGUGCAACGCCUUCUGCCCGUGCUGUGCCAGGCGCAUGGUC |
| | | UGACCCCUGAGCAAGUGGUCGCCAUCGCAUCCAACAUCGGGGGCA |
| | | AACAAGCCCUGGAGACUGUGCAGAGGCUCCUGCCGGUGCUCUGCC |
| | | AAGCCCACGGGCUCACCCCCGAACAAGUCGUGGCCAUCGCCUCCA |
| | | ACAACGGCGGGAAGCAGGCUCUGGAAACGGUGCAGAGACUGCUCC |
| | | CUGUGCUGUGUCAGGCCCACGGACUCACCCCUGAACAGGUUGUCG |
| | | CAAUCGCGUCGAAUGGAGGGGGAAAGCAGGCACUGGAGACAGUGC |
| | | AGCGCCUGCUCCCCGUGCUUUGCCAAGCUCACGGACUUACCCCGG |
| | | AACAGGUCGUGGCAAUCGCCUCAAACAACGGUGGACGCCCGGCCC |
| | | UGGAGAGCAUCGUGGCCCAGCUCUCCCGCCCCGAUCCGGCCCUGG |
| | | CUGCCCUCACUAACGACCACCUGGUCGCCCUGGCCUGCCUGGGCG |
| | | GACGGCCAGCCCUGGACGCCGUGAAGAAGGGACUGCCCCACGCCC |
| | | CCGCCCUGAUUAAGCGGACCAACCGCAGGAUUCCUGAGAGAACCU |
| | | CCCAUCGGGUGGCAUCUGGGAGCGGAGGAGGAUCCGGCGGAGCCA |
| | | GAGACGACGCCAAGAGCCUGACCGCCUGGAGCCGGACCCUGGUGA |
| | | CCUUCAAGGACGUGUUCGUGGACUUCACCCGGGAGGAGUGGAAGC |
| | | UGCUGGACACCGCCCAGCAGAUCCUGUACCGGAACGUGAUGCUGG |
| | | AGAACUACAAGAACCUGGUGAGCCUGGGCUACCAGCUGACCAAGC |
| | | CCGACGUGAUCCUGCGGCUGGAGAAGGGCGAGGAGCCCUGGCUGG |
| | | UGGAGCGGGAGAUCCACCAGGAGACCCACCCCGACAGCGAGACCG |
| | | CCUUCGAGAUCAAGAGCAGCGUGCCUUCCUCCGGUGGAAAGCGCC |
| | | CAGCGGCUACAAAGAAGGCCGGACAGGCCAAGAAGAAGAAGGGUU |
| | | CGUACCCCUACGACGUGCCUGACUAUGCCUGAGCGGCCGCUUAAU |
| | | UAAGCUGCCUUCUGCGGGGCUUGCCUUCUGGCCAUGCCCUUCUUC |
| | | UCUCCCUUGCACCUGUACCUCUUGGUCUUUGAAUAAAGCCUGAGU |
| | | AGGAAGUCUAGAAAAAAAAAAAAAAAAAAAAAAAAAAAAAAAAAA |
| | | AAAAAAAAAAAAAAAAAAAAAAAAAAAAAAAAAAAAAAAAAAAAA |
| | | AAAAAAAAAAAAAAAAAAAAAAAAAAAAAAAA |
| 315 | Mouse CXCL | CACAUGGUCGACCUGAGGACCCUCGGAUACUCGCAGCAGCAGCAG |
| | TAL16-KRAB | GAAAAGAUCAAGCCUAAGGUCCGCAGCACCGUGGCUCAGCAUCAU |
| | DBD RNA | GAGGCCCUUGUGGGACACGGGUUCACCCACGCCCAUAUUGUGGCC |
| | sequence | CUGUCCCAACACCCGGCUGCACUGGGCACCGUGGCCGUGAAGUAC |
| | (from | CAGGACAUGAUCGCCGCCCUGCCCGAGGCGACCCACGAGGCCAUC |
| | MR-33720) | GUGGGAGUGGGAAAGCGCGGCGCCGGUGCCAGAGCACUGGAAGCG |
| | | CUGCUGACUGUGGCGGGAGAGCUGAGGGGACCUCCACUCCAAUUG |
| | | GAUACCGGGCAGCUCCUGAAGAUCGCCAAGCGCGGCGGAGUGACC |
| | | GCCGUGGAAGCCGUGCACGCCUGGCGGAACGCGCUGACUGGGGCC |
| | | CCGCUGAACCUGACCCCAGAACAGGUUGUGGCUAUCGCCUCCAAC |
| | | AACGGCGGAAAACAGGCCUUGGAGACUGUGCAGCGGCUUCUGCCG |
| | | GUGCUUUGUCAAGCCCACGGCCUGACCCCGGAACAGGUCGUGGCC |
| | | AUCGCCUCAAACGGCGGCGGGAAGCAGGCUCUCGAAACCGUGCAA |
| | | CGGCUCCUGCCCGUGCUGUGCCAAGCUCAUGGUCUGACACCUGAA |
| | | CAAGUGGUGGCGAUUGCGUCCCACGACGGGGGCAAACAGGCCCUC |
| | | GAAACUGUCCAGCGGCUGCUGCCCGUGUUGUGCCAAGCACACGGA |
| | | CUCACGCCCGAACAAGUCGUCGCGAUUGCAAGCAAUGGUGGUGGA |
| | | AAGCAGGCCCUGGAGACUGUCCAGAGACUCCUGCCCGUGCUGUGC |
| | | CAGGCUCACGGGCUGACCCCGGAACAAGUCGUGGCCAUUGCCAGC |
| | | AACAUCGGCGGCAAACAGGCUCUGGAGACUGUGCAGCGCCUGCUG |
| | | CCAGUGCUGUGUCAGGCGCACGGACUGACCCCCGAACAGGUCGUG |
| | | GCAAUCGCAUCAAACAUCGGAGGAAAACAGGCCUUGGAAACCGUC |
| | | CAGCGGCUGUUGCCCGUGCUUUGCCAAGCUCAUGGACUCACCCCU |
| | | GAGCAAGUCGUCGCGAUUGCCUCCAACGGUGGAGGAAAGCAAGCC |
| | | CUUGAGACUGUCCAGAGGCUGCUGCCGGUGUUAUGCCAGGCCCAC |
| | | GGUCUGACCCCAGAACAGGUGGUGGCCAUCGCCAGCCAUGACGGU |
| | | GGCAAACAAGCCCUCGAAACAGUGCAGAGACUGCUCCCGGUCCUG |
| | | UGCCAAGCCCACGGCCUCACCCCGGAGCAAGUGGUGGCUAUCGCC |
| | | UCGCACGAUGGAGGGAAGCAAGCGCUGGAAACCGUGCAGCGGUUG |
| | | CUGCCCGUCCUGUGUCAAGCGCACGGCCUGACUCCGGAACAGGUA |
| | | GUCGCCAUUGCGUCCAACGGCGGCGGGAAGCAGGCGCUCGAAACG |
| | | GUGCAGCGGCUGCUGCCUGUGCUCUGCCAAGCACACGGACUUACC |
| | | CCUGAACAAGUGGUCGCCAUCGCCUCCAAUGGUGGCGGAAAGCAG |
| | | GCCCUGGAAACUGUGCAACGCCUCCUGCCCGUGUUGUGCCAGGCG |
| | | CAUGGGCUCACUCCUGAACAGGUUGUCGCAAUCGCUUCCAACAAC |
| | | GGCGGGAAACAGGCACUGGAAACCGUGCAGCGGCUGUUGCCAGUG |
| | | UUGUGCCAAGCGCACGGCCUGACUCCUGAACAAGUGGUGGCCAUU |
| | | GCGUCGCGGCAUGGUGGCAAACAGGCCCUCGAAACUGUGCAACGG |
| | | CUGCUGCCGGUGUUGUGUCAGGCGCACGGACUGACCCCGGAGCAA |
| | | GUCGUCGCUAUCGCGAGCAGACACGGCGGCAAGCAGGCCCUGGAA |
| | | ACCGUGCAACGCCUUCUGCCCGUGCUGUGCCAGGCGCAUGGUCUG |
| | | ACCCCUGAGCAAGUGGUCGCCAUCGCAUCCAACAUCGGGGGCAAA |
| | | CAAGCCCUGGAGACUGUGCAGAGGCUCCUGCCGGUGCUCUGCCAA |
| | | GCCCACGGGCUCACCCCCGAACAAGUCGUGGCCAUCGCCUCCAAC |
| | | AACGGCGGGAAGCAGGCUCUGGAAACGGUGCAGAGACUGCUCCCU |
| | | GUGCUGUGUCAGGCCCACGGACUCACCCCUGAACAGGUUGUCGCA |
| | | AUCGCGUCGAAUGGAGGGGGAAAGCAGGCACUGGAGACAGUGCAG |
| | | CGCCUGCUCCCCGUGCUUUGCCAAGCUCACGGACUUACCCCGGAA |
| | | CAGGUCGUGGCAAUCGCCUCAAACAACGGUGGACGCCCGGCCCUG |
| | | GAGAGCAUCGUGGCCCAGCUCUCCCGCCCCGAUCCGGCCCUGGCU |
| | | GCCCUCACUAACGACCACCUGGUCGCCCUGGCCUGCCUGGGCGGA |
| | | CGGCCAGCCCUGGACGCCGUGAAGAAGGGACUGCCCCACGCCCCC |
| | | GCCCUGAUUAAGCGGACCAACCGCAGGAUUCCUGAGAGAACCUCC |
| | | CAUCGGGUG |
| 317 | Mouse CXCL | GACGCCAAGAGCCUGACCGCCUGGAGCCGGACCCUGGUGACCUUC |
| | TAL16-KRAB | AAGGACGUGUUCGUGGACUUCACCCGGGAGGAGUGGAAGCUGCUG |
| | Effector RNA | GACACCGCCCAGCAGAUCCUGUACCGGAACGUGAUGCUGGAGAAC |
| | sequence (from | UACAAGAACCUGGUGAGCCUGGGCUACCAGCUGACCAAGCCCGAC |
| | MR-33720) | GUGAUCCUGCGGCUGGAGAAGGGCGAGGAGCCCUGGCUGGUGGAG |
| | | CGGGAGAUCCACCAGGAGACCCACCCCGACAGCGAGACCGCCUUC |
| | | GAGAUCAAGAGCAGCGUG |
| 320 | Mouse CXCL | GGGAAAUAAGAGAGAAAAGAAGAGUAAGAAGAAAUAUAAGAGCCA |
| | TAL17-KRAB | CCAUGGCCCCAAAGAAGAAGCGGAAAGUCGGAGGUUCAGGCGGCU |
| | mRNA sequence | CGCACAUGGUCGACCUUCGGACCCUCGGAUACUCGCAGCAGCAGC |
| | (MR-33721) | AAGAGAAGAUCAAGCCCAAGGUCCGCUCCACCGUGGCCCAGCACC |
| | | ACGAAGCCCUGGUCGGCCACGGUUUCACUCACGCCCACAUUGUGG |
| | | CGCUGUCCCAGCAUCCUGCCGCACUCGGGACCGUGGCUGUGAAGU |
| | | ACCAGGACAUGAUCGCGGCCUUGCCCGAGGCCACCCAUGAAGCCA |
| | | UCGUGGGAGUGGGGAAGAGAGGCGCCGGAGCCAGGGCCCUUGAGG |
| | | CCCUGCUCACCGUGGCUGGAGAGCUGCGGGGACCGCCGCUGCAAC |
| | | UGGAUACGGGACAGCUGCUGAAGAUCGCAAAGCGCGGUGGAGUGA |
| | | CAGCGGUCGAGGCCGUGCAUGCCUGGCGGAACGCCCUCACCGGCG |
| | | CCCCUCUCAACCUGACCCCUGAACAAGUGGUCGCCAUCGCGAGCC |
| | | ACGACGGCGGGAAACAGGCUCUUGAAACCGUCCAACGCUUGCUGC |
| | | CCGUGCUGUGUCAGGCGCAUGGGCUGACUCCGGAACAGGUUGUGG |
| | | CUAUUGCCAGCAAUGGCGGCGGAAAGCAGGCCUUGGAAACUGUGC |
| | | AGAGGCUGCUGCCGGUGCUCUGCCAAGCUCACGGUCUGACCCCCG |
| | | AACAAGUCGUGGCCAUCGCAUCCAACAACGGCGGGAAGCAGGCAC |
| | | UCGAAACCGUGCAGAGACUGCUGCCAGUGCUGUGCCAAGCCCACG |
| | | GACUGACUCCAGAACAGGUCGUGGCGAUUGCCUCAAACGGUGGCG |
| | | GAAAACAAGCGCUGGAGACUGUGCAGCGGCUGCUCCCGGUGUUGU |
| | | GUCAAGCGCACGGCCUGACCCCCGAGCAAGUGGUCGCCAUCGCCA |
| | | GCAACAUCGGUGGCAAACAGGCACUCGAAACCGUCCAGCGGUUGC |
| | | UGCCAGUGCUGUGCCAAGCCCACGGACUUACCCCGGAACAAGUCG |
| | | UCGCGAUUGCCAGCAACAACGGCGGCAAGCAGGCUCUGGAAACCG |
| | | UGCAGAGACUGCUGCCCGUGUUGUGCCAGGCGCACGGACUGACCC |
| | | CUGAACAGGUCGUGGCAAUUGCCUCCAAUGGUGGCGGCAAACAAG |
| | | CUCUGGAGACUGUGCAGAGGCUGCUGCCUGUCCUGUGCCAGGCAC |
| | | ACGGUCUGACUCCUGAACAAGUGGUGGCGAUUGCCUCAAACAUCG |
| | | GAGGGAAGCAGGCCCUGGAGACAGUGCAGCGCCUGCUGCCGGUGC |
| | | UUUGCCAAGCUCAUGGACUGACUCCAGAACAGGUUGUGGCCAUUG |
| | | CCUCCCAUGACGGAGGGAAACAAGCCCUGGAAACGGUGCAGCGGU |
| | | UACUCCCGGUGCUCUGUCAGGCGCAUGGGCUGACCCCGGAGCAAG |
| | | UCGUGGCUAUCGCCUCCAACAUCGGCGGGAAGCAAGCCCUCGAAA |
| | | CUGUGCAGCGGCUGCUCCCUGUGCUCUGCCAAGCGCAUGGUCUGA |
| | | CGCCCGAACAGGUGGUGGCCAUCGCCUCCCACGACGGAGGAAAGC |
| | | AGGCCUUGGAGACUGUCCAACGCCUGCUCCCCGUGCUGUGUCAGG |
| | | CCCACGGGCUUACCCCCGAACAAGUUGUGGCAAUCGCCUCGCACG |
| | | AUGGCGGAAAACAGGCCCUCGAAACCGUGCAGCGCCUCCUCCCGG |
| | | UCCUGUGCCAAGCGCAUGGACUGACCCCCGAACAGGUGGUCGCAA |
| | | UUGCCUCCAACAUUGGCGGGAAACAGGCUCUGGAGACUGUGCAGC |
| | | GGCUGCUGCCUGUGCUUUGCCAAGCUCACGGACUCACGCCGGAAC |
| | | AAGUCGUGGCCAUCGCGUCCAAUAUCGGCGGAAAGCAGGCCCUCG |
| | | AAACCGUCCAGAGACUUCUCCCGGUGCUCUGUCAAGCGCACGGGC |
| | | UGACCCCAGAACAGGUUGUGGCCAUUGCGUCCAACAACGGAGGAA |
| | | AACAGGCCCUGGAGACAGUGCAGAGACUGCUGCCGGUGUUGUGCC |
| | | AGGCGCACGGAUUGACCCCUGAGCAAGUGGUGGCAAUCGCCUCAA |
| | | ACGGCGGCGGAAAACAAGCCCUGGAAACUGUGCAACGGCUGCUCC |
| | | CUGUGCUGUGCCAGGCACAUGGUCUGACCCCGGAACAGGUCGUGG |
| | | CGAUUGCCUCGAACAACGGCGGGAAGCAGGCACUUGAGACUGUCC |
| | | AGAGGCUGCUCCCCGUGCUGUGUCAGGCCCACGGACUGACUCCCG |
| | | AACAAGUGGUCGCCAUCGCCUCGCGCCACGGGGGGCGCCCAGCCC |
| | | UGGAGAGCAUCGUGGCCCAGCUGUCCCGGCCCGACCCUGCCCUGG |
| | | CUGCCCUGACCAACGACCACUUGGUGGCCCUCGCCUGCCUGGGUG |
| | | GCAGACCGGCCCUGGAUGCUGUGAAGAAGGGUCUGCCUCAUGCCC |
| | | CCGCCCUGAUCAAGCGGACCAACCGGCGCAUCCCCGAGAGGACCA |
| | | GCCACCGGGUGGCAUCUGGGAGCGGAGGAGGAUCCGGCGGAGCCA |
| | | GAGACGACGCCAAGAGCCUGACCGCCUGGAGCCGGACCCUGGUGA |
| | | CCUUCAAGGACGUGUUCGUGGACUUCACCCGGGAGGAGUGGAAGC |
| | | UGCUGGACACCGCCCAGCAGAUCCUGUACCGGAACGUGAUGCUGG |
| | | AGAACUACAAGAACCUGGUGAGCCUGGGCUACCAGCUGACCAAGC |
| | | CCGACGUGAUCCUGCGGCUGGAGAAGGGCGAGGAGCCCUGGCUGG |
| | | UGGAGCGGGAGAUCCACCAGGAGACCCACCCCGACAGCGAGACCG |
| | | CCUUCGAGAUCAAGAGCAGCGUGCCUUCCUCCGGUGGAAAGCGCC |
| | | CAGCGGCUACAAAGAAGGCCGGACAGGCCAAGAAGAAGAAGGGUU |
| | | CGUACCCCUACGACGUGCCUGACUAUGCCUGAGCGGCCGCUUAAU |
| | | UAAGCUGCCUUCUGCGGGGCUUGCCUUCUGGCCAUGCCCUUCUUC |
| | | UCUCCCUUGCACCUGUACCUCUUGGUCUUUGAAUAAAGCCUGAGU |
| | | AGGAAGUCUAGAAAAAAAAAAAAAAAAAAAAAAAAAAAAAAAAAA |
| | | AAAAAAAAAAAAAAAAAAAAAAAAAAAAAAAAAAAAAAAAAAAAA |
| | | AAAAAAAAAAAAAAAAAAAAAAAAAAAAAAAA |
| 322 | Mouse CXCL | CACAUGGUCGACCUUCGGACCCUCGGAUACUCGCAGCAGCAGCAA |
| | TAL17-KRAB | GAGAAGAUCAAGCCCAAGGUCCGCUCCACCGUGGCCCAGCACCAC |
| | DBD RNA | GAAGCCCUGGUCGGCCACGGUUUCACUCACGCCCACAUUGUGGCG |
| | sequence (from | CUGUCCCAGCAUCCUGCCGCACUCGGGACCGUGGCUGUGAAGUAC |
| | MR-33721) | CAGGACAUGAUCGCGGCCUUGCCCGAGGCCACCCAUGAAGCCAUC |
| | | GUGGGAGUGGGGAAGAGAGGCGCCGGAGCCAGGGCCCUUGAGGCC |
| | | CUGCUCACCGUGGCUGGAGAGCUGCGGGGACCGCCGCUGCAACUG |
| | | GAUACGGGACAGCUGCUGAAGAUCGCAAAGCGCGGUGGAGUGACA |
| | | GCGGUCGAGGCCGUGCAUGCCUGGCGGAACGCCCUCACCGGCGCC |
| | | CCUCUCAACCUGACCCCUGAACAAGUGGUCGCCAUCGCGAGCCAC |
| | | GACGGCGGGAAACAGGCUCUUGAAACCGUCCAACGCUUGCUGCCC |
| | | GUGCUGUGUCAGGCGCAUGGGCUGACUCCGGAACAGGUUGUGGCU |
| | | AUUGCCAGCAAUGGCGGCGGAAAGCAGGCCUUGGAAACUGUGCAG |
| | | AGGCUGCUGCCGGUGCUCUGCCAAGCUCACGGUCUGACCCCCGAA |
| | | CAAGUCGUGGCCAUCGCAUCCAACAACGGCGGGAAGCAGGCACUC |
| | | GAAACCGUGCAGAGACUGCUGCCAGUGCUGUGCCAAGCCCACGGA |
| | | CUGACUCCAGAACAGGUCGUGGCGAUUGCCUCAAACGGUGGCGGA |
| | | AAACAAGCGCUGGAGACUGUGCAGCGGCUGCUCCCGGUGUUGUGU |
| | | CAAGCGCACGGCCUGACCCCCGAGCAAGUGGUCGCCAUCGCCAGC |
| | | AACAUCGGUGGCAAACAGGCACUCGAAACCGUCCAGCGGUUGCUG |
| | | CCAGUGCUGUGCCAAGCCCACGGACUUACCCCGGAACAAGUCGUC |
| | | GCGAUUGCCAGCAACAACGGCGGCAAGCAGGCUCUGGAAACCGUG |
| | | CAGAGACUGCUGCCCGUGUUGUGCCAGGCGCACGGACUGACCCCU |
| | | GAACAGGUCGUGGCAAUUGCCUCCAAUGGUGGCGGCAAACAAGCU |
| | | CUGGAGACUGUGCAGAGGCUGCUGCCUGUCCUGUGCCAGGCACAC |
| | | GGUCUGACUCCUGAACAAGUGGUGGCGAUUGCCUCAAACAUCGGA |
| | | GGGAAGCAGGCCCUGGAGACAGUGCAGCGCCUGCUGCCGGUGCUU |
| | | UGCCAAGCUCAUGGACUGACUCCAGAACAGGUUGUGGCCAUUGCC |
| | | UCCCAUGACGGAGGGAAACAAGCCCUGGAAACGGUGCAGCGGUUA |
| | | CUCCCGGUGCUCUGUCAGGCGCAUGGGCUGACCCCGGAGCAAGUC |
| | | GUGGCUAUCGCCUCCAACAUCGGCGGGAAGCAAGCCCUCGAAACU |
| | | GUGCAGCGGCUGCUCCCUGUGCUCUGCCAAGCGCAUGGUCUGACG |
| | | CCCGAACAGGUGGUGGCCAUCGCCUCCCACGACGGAGGAAAGCAG |
| | | GCCUUGGAGACUGUCCAACGCCUGCUCCCCGUGCUGUGUCAGGCC |
| | | CACGGGCUUACCCCCGAACAAGUUGUGGCAAUCGCCUCGCACGAU |
| | | GGCGGAAAACAGGCCCUCGAAACCGUGCAGCGCCUCCUCCCGGUC |
| | | CUGUGCCAAGCGCAUGGACUGACCCCCGAACAGGUGGUCGCAAUU |
| | | GCCUCCAACAUUGGCGGGAAACAGGCUCUGGAGACUGUGCAGCGG |
| | | CUGCUGCCUGUGCUUUGCCAAGCUCACGGACUCACGCCGGAACAA |
| | | GUCGUGGCCAUCGCGUCCAAUAUCGGCGGAAAGCAGGCCCUCGAA |
| | | ACCGUCCAGAGACUUCUCCCGGUGCUCUGUCAAGCGCACGGGCUG |
| | | ACCCCAGAACAGGUUGUGGCCAUUGCGUCCAACAACGGAGGAAAA |
| | | CAGGCCCUGGAGACAGUGCAGAGACUGCUGCCGGUGUUGUGCCAG |
| | | GCGCACGGAUUGACCCCUGAGCAAGUGGUGGCAAUCGCCUCAAAC |
| | | GGCGGCGGAAAACAAGCCCUGGAAACUGUGCAACGGCUGCUCCCU |
| | | GUGCUGUGCCAGGCACAUGGUCUGACCCCGGAACAGGUCGUGGCG |
| | | AUUGCCUCGAACAACGGCGGGAAGCAGGCACUUGAGACUGUCCAG |
| | | AGGCUGCUCCCCGUGCUGUGUCAGGCCCACGGACUGACUCCCGAA |
| | | CAAGUGGUCGCCAUCGCCUCGCGCCACGGCGGGCGCCCAGCCCUG |
| | | GAGAGCAUCGUGGCCCAGCUGUCCCGGCCCGACCCUGCCCUGGCU |
| | | GCCCUGACCAACGACCACUUGGUGGCCCUCGCCUGCCUGGGUGGC |
| | | AGACCGGCCCUGGAUGCUGUGAAGAAGGGUCUGCCUCAUGCCCCC |
| | | GCCCUGAUCAAGCGGACCAACCGGCGCAUCCCCGAGAGGACCAGC |
| | | CACCGGGUG |
| 324 | Mouse CXCL | GACGCCAAGAGCCUGACCGCCUGGAGCCGGACCCUGGUGACCUUC |
| | TAL17-KRAB | AAGGACGUGUUCGUGGACUUCACCCGGGAGGAGUGGAAGCUGCUG |
| | Effector RNA | GACACCGCCCAGCAGAUCCUGUACCGGAACGUGAUGCUGGAGAAC |
| | sequence (from | UACAAGAACCUGGUGAGCCUGGGCUACCAGCUGACCAAGCCCGAC |
| | MR-33721) | GUGAUCCUGCGGCUGGAGAAGGGCGAGGAGCCCUGGCUGGUGGAG |
| | | CGGGAGAUCCACCAGGAGACCCACCCCGACAGCGAGACCGCCUUC |
| | | GAGAUCAAGAGCAGCGUG |
| 327 | Mouse CXCL | GGGAAAUAAGAGAGAAAAGAAGAGUAAGAAGAAAUAUAAGAGCCA |
| | TAL18-KRAB | CCAUGGCCCCAAAGAAGAAGCGGAAAGUCGGAGGUUCAGGCGGCU |
| | mRNA sequence | CGCACAUGGUGGACCUCAGGACCCUGGGAUACAGCCAGCAGCAGC |
| | (MR-33722) | AGGAAAAGAUCAAGCCCAAGGUCCGCAGCACAGUGGCUCAGCACC |
| | | AUGAGGCCCUUGUGGGACACGGAUUCACCCACGCGCACAUCGUGG |
| | | CACUGUCCCAACACCCUGCGGCCCUGGGGACCGUGGCCGUGAAGU |
| | | ACCAGGAUAUGAUCGCCGCUCUGCCCGAGGCCACCCAUGAAGCCA |
| | | UCGUGGGAGUGGGAAAGCGCGGAGCGGGCGCCAGAGCCCUUGAGG |
| | | CCCUGCUCACUGUGGCCGGCGAACUGCGGGGUCCUCCGCUGCAAU |
| | | UGGACACCGGGCAGCUCCUGAAGAUUGCUAAGCGCGGUGGAGUGA |
| | | CCGCCGUCGAGGCCGUGCAUGCGUGGCGGAACGCCCUCACUGGUG |
| | | CCCCGCUGAACCUGACCCCAGAACAAGUUGUGGCCAUCGCAUCCA |
| | | ACAACGGCGGGAAGCAGGCCCUCGAAACCGUGCAGCGGCUGCUGC |
| | | CAGUGCUGUGUCAGGCCCACGGGCUGACCCCGGAACAGGUCGUGG |
| | | CAAUCGCCUCCAAUGGCGGCGGGAAACAGGCCCUUGAGACUGUCC |
| | | AGAGACUGCUCCCGGUCCUGUGCCAAGCACACGGACUGACGCCCG |
| | | AACAAGUGGUGGCUAUUGCCUCAAACGGCGGCGGAAAGCAGGCUC |
| | | UGGAAACCGUCCAGAGGCUCCUGCCCGUGCUCUGCCAAGCGCACG |
| | | GCUUGACUCCUGAACAAGUCGUGGCGAUUGCGUCGAACAUCGGUG |
| | | GAAAACAAGCCCUGGAGACUGUGCAGCGGUUGCUGCCCGUGCUGU |
| | | GUCAGGCGCACGGGCUCACCCCCGAACAGGUCGUGGCCAUCGCCU |
| | | CAAACAACGGCGGAAAGCAGGCCCUGGAAACUGUGCAACGCCUGC |
| | | UGCCUGUGUUGUGUCAGGCCCACGGCCUCACUCCCGAACAAGUUG |
| | | UGGCUAUCGCCUCGCGGCACGGAGGGAAGCAAGCCCUGGAGACUG |
| | | UCCAGAGACUGCUGCCCGUGCUUUGUCAAGCCCACGGACUCACCC |
| | | CUGAGCAAGUGGUGGCCAUUGCAUCCAAUGGUGGUGGCAAACAGG |
| | | CUCUCGAAACAGUGCAGAGACUCCUCCCCGUGCUCUGCCAAGCGC |
| | | ACGGACUGACUCCUGAACAAGUAGUCGCCAUUGCCUCCCACGAUG |
| | | GCGGGAAACAGGCCCUCGAAACUGUCCAGCGGCUGUUGCCGGUGU |
| | | UGUGCCAGGCACAUGGUCUGACCCCGGAGCAAGUCGUGGCUAUUG |
| | | CGUCCAACGGCGGAGGAAAGCAAGCCUUGGAGACUGUGCAGCGCC |
| | | UGCUUCCUGUGCUUUGCCAAGCUCACGGACUUACCCCGGAACAAG |
| | | UCGUCGCAAUCGCCAGCCAUGACGGCGGGAAGCAGGCUCUGGAAA |
| | | CCGUGCAGCGGCUGCUGCCAGUGCUGUGCCAGGCGCAUGGACUGA |
| | | CCCCAGAACAAGUGGUCGCCAUCGCGAGCCACGACGGCGGCAAGC |
| | | AGGCCUUAGAAACCGUCCAGAGGCUGCUGCCGGUCCUGUGCCAAG |
| | | CACACGGUCUGACGCCGGAACAGGUGGUCGCGAUUGCCUCCAACA |
| | | UCGGGGGAAAACAGGCGCUGGAGACCGUGCAGAGACUCCUGCCUG |
| | | UGUUAUGCCAAGCCCACGGCCUGACUCCCGAACAAGUCGUCGCCA |
| | | UUGCGUCACACGACGGCGGGAAGCAGGCCCUUGAGACAGUGCAGC |
| | | GACUCCUGCCCGUGCUGUGCCAGGCUCAUGGACUCACCCCGGAAC |
| | | AGGUUGUGGCCAUCGCCUCCAAUGGUGGCGGCAAACAGGCCCUCG |
| | | AAACCGUCCAGCGGCUCCUCCCGGUCCUGUGCCAAGCUCAUGGAU |
| | | UGACCCCUGAGCAAGUCGUGGCAAUCGCCUCGAACAACGGCGGAA |
| | | AGCAGGCCUUGGAGACUGUGCAGAGGCUGCUGCCUGUGCUGUGUC |
| | | AGGCCCACGGACUGACCCCGGAGCAAGUGGUGGCGAUUGCCUCCA |
| | | ACAUCGGCGGAAAACAAGCCCUGGAAACCGUGCAGCGCCUUCUGC |
| | | CCGUGUUGUGCCAGGCGCACGGUCUGACCCCUGAACAAGUGGUCG |
| | | CCAUCGCGAGCAACGGAGGGGGAAAACAGGCUCUGGAAACUGUGC |
| | | AGCGGCUGCUCCCGGUGCUCUGUCAAGCACACGGGCUGACCCCAG |
| | | AACAGGUCGUGGCCAUUGCGAGCAACAACGGUGGACGCCCUGCCC |
| | | UUGAGUCCAUCGUGGCCCAGCUGUCGAGGCCCGACCCGGCCCUGG |
| | | CUGCGCUGACUAACGACCACCUGGUGGCCCUCGCCUGCCUGGGCG |
| | | GGCGGCCCGCCCUGGAUGCAGUGAAGAAGGGACUGCCGCAUGCAC |
| | | CAGCCCUGAUCAAGCGGACCAACCGCAGAAUCCCCGAGCGCACGU |
| | | CCCACCGGGUGGCAUCUGGGAGCGGAGGAGGAUCCGGCGGAGCCA |
| | | GAGACGACGCCAAGAGCCUGACCGCCUGGAGCCGGACCCUGGUGA |
| | | CCUUCAAGGACGUGUUCGUGGACUUCACCCGGGAGGAGUGGAAGC |
| | | UGCUGGACACCGCCCAGCAGAUCCUGUACCGGAACGUGAUGCUGG |
| | | AGAACUACAAGAACCUGGUGAGCCUGGGCUACCAGCUGACCAAGC |
| | | CCGACGUGAUCCUGCGGCUGGAGAAGGGCGAGGAGCCCUGGCUGG |
| | | UGGAGCGGGAGAUCCACCAGGAGACCCACCCCGACAGCGAGACCG |
| | | CCUUCGAGAUCAAGAGCAGCGUGCCUUCCUCCGGUGGAAAGCGCC |
| | | CAGCGGCUACAAAGAAGGCCGGACAGGCCAAGAAGAAGAAGGGUU |
| | | CGUACCCCUACGACGUGCCUGACUAUGCCUGAGCGGCCGCUUAAU |
| | | UAAGCUGCCUUCUGCGGGGCUUGCCUUCUGGCCAUGCCCUUCUUC |
| | | UCUCCCUUGCACCUGUACCUCUUGGUCUUUGAAUAAAGCCUGAGU |
| | | AGGAAGUCUAGAAAAAAAAAAAAAAAAAAAAAAAAAAAAAAAAAA |
| | | AAAAAAAAAAAAAAAAAAAAAAAAAAAAAAAAAAAAAAA |
| | | AAAAAAAAAAAAAAAAAAAAA |
| 329 | Mouse CXCL | CACAUGGUGGACCUCAGGACCCUGGGAUACAGCCAGCAGCAGCAG |
| | TAL18-KRAB | GAAAAGAUCAAGCCCAAGGUCCGCAGCACAGUGGCUCAGCACCAU |
| | DBD RNA | GAGGCCCUUGUGGGACACGGAUUCACCCACGCGCACAUCGUGGCA |
| | sequence (from | CUGUCCCAACACCCUGCGGCCCUGGGGACCGUGGCCGUGAAGUAC |
| | MR-33722) | CAGGAUAUGAUCGCCGCUCUGCCCGAGGCCACCCAUGAAGCCAUC |
| | | GUGGGAGUGGGAAAGCGCGGAGCGGGCGCCAGAGCCCUUGAGGCC |
| | | CUGCUCACUGUGGCCGGCGAACUGCGGGGUCCUCCGCUGCAAUUG |
| | | GACACCGGGCAGCUCCUGAAGAUUGCUAAGCGCGGUGGAGUGACC |
| | | GCCGUCGAGGCCGUGCAUGCGUGGCGGAACGCCCUCACUGGUGCC |
| | | CCGCUGAACCUGACCCCAGAACAAGUUGUGGCCAUCGCAUCCAAC |
| | | AACGGCGGGAAGCAGGCCCUCGAAACCGUGCAGCGGCUGCUGCCA |
| | | GUGCUGUGUCAGGCCCACGGGCUGACCCCGGAACAGGUCGUGGCA |
| | | AUCGCCUCCAAUGGCGGCGGGAAACAGGCCCUUGAGACUGUCCAG |
| | | AGACUGCUCCCGGUCCUGUGCCAAGCACACGGACUGACGCCCGAA |
| | | CAAGUGGUGGCUAUUGCCUCAAACGGCGGCGGAAAGCAGGCUCUG |
| | | GAAACCGUCCAGAGGCUCCUGCCCGUGCUCUGCCAAGCGCACGGC |
| | | UUGACUCCUGAACAAGUCGUGGCGAUUGCGUCGAACAUCGGUGGA |
| | | AAACAAGCCCUGGAGACUGUGCAGCGGUUGCUGCCCGUGCUGUGU |
| | | CAGGCGCACGGGCUCACCCCCGAACAGGUCGUGGCCAUCGCCUCA |
| | | AACAACGGCGGAAAGCAGGCCCUGGAAACUGUGCAACGCCUGCUG |
| | | CCUGUGUUGUGUCAGGCCCACGGCCUCACUCCCGAACAAGUUGUG |
| | | GCUAUCGCCUCGCGGCACGGAGGGAAGCAAGCCCUGGAGACUGUC |
| | | CAGAGACUGCUGCCCGUGCUUUGUCAAGCCCACGGACUCACCCCU |
| | | GAGCAAGUGGUGGCCAUUGCAUCCAAUGGUGGUGGCAAACAGGCU |
| | | CUCGAAACAGUGCAGAGACUCCUCCCCGUGCUCUGCCAAGCGCAC |
| | | GGACUGACUCCUGAACAAGUAGUCGCCAUUGCCUCCCACGAUGGC |
| | | GGGAAACAGGCCCUCGAAACUGUCCAGCGGCUGUUGCCGGUGUUG |
| | | UGCCAGGCACAUGGUCUGACCCCGGAGCAAGUCGUGGCUAUUGCG |
| | | UCCAACGGCGGAGGAAAGCAAGCCUUGGAGACUGUGCAGCGCCUG |
| | | CUUCCUGUGCUUUGCCAAGCUCACGGACUUACCCCGGAACAAGUC |
| | | GUCGCAAUCGCCAGCCAUGACGGCGGGAAGCAGGCUCUGGAAACC |
| | | GUGCAGCGGCUGCUGCCAGUGCUGUGCCAGGCGCAUGGACUGACC |
| | | CCAGAACAAGUGGUCGCCAUCGCGAGCCACGACGGCGGCAAGCAG |
| | | GCCUUAGAAACCGUCCAGAGGCUGCUGCCGGUCCUGUGCCAAGCA |
| | | CACGGUCUGACGCCGGAACAGGUGGUCGCGAUUGCCUCCAACAUC |
| | | GGGGGAAAACAGGCGCUGGAGACCGUGCAGAGACUCCUGCCUGUG |
| | | UUAUGCCAAGCCCACGGCCUGACUCCCGAACAAGUCGUCGCCAUU |
| | | GCGUCACACGACGGCGGGAAGCAGGCCCUUGAGACAGUGCAGCGA |
| | | CUCCUGCCCGUGCUGUGCCAGGCUCAUGGACUCACCCCGGAACAG |
| | | GUUGUGGCCAUCGCCUCCAAUGGUGGCGGCAAACAGGCCCUCGAA |
| | | ACCGUCCAGCGGCUCCUCCCGGUCCUGUGCCAAGCUCAUGGAUUG |
| | | ACCCCUGAGCAAGUCGUGGCAAUCGCCUCGAACAACGGCGGAAAG |
| | | CAGGCCUUGGAGACUGUGCAGAGGCUGCUGCCUGUGCUGUGUCAG |
| | | GCCCACGGACUGACCCCGGAGCAAGUGGUGGCGAUUGCCUCCAAC |
| | | AUCGGCGGAAAACAAGCCCUGGAAACCGUGCAGCGCCUUCUGCCC |
| | | GUGUUGUGCCAGGCGCACGGUCUGACCCCUGAACAAGUGGUCGCC |
| | | AUCGCGAGCAACGGAGGGGGAAAACAGGCUCUGGAAACUGUGCAG |
| | | CGGCUGCUCCCGGUGCUCUGUCAAGCACACGGGCUGACCCCAGAA |
| | | CAGGUCGUGGCCAUUGCGAGCAACAACGGUGGACGCCCUGCCCUU |
| | | GAGUCCAUCGUGGCCCAGCUGUCGAGGCCCGACCCGGCCCUGGCU |
| | | GCGCUGACUAACGACCACCUGGUGGCCCUCGCCUGCCUGGGCGGG |
| | | CGGCCCGCCCUGGAUGCAGUGAAGAAGGGACUGCCGCAUGCACCA |
| | | GCCCUGAUCAAGCGGACCAACCGCAGAAUCCCCGAGCGCACGUCC |
| | | CACCGGGUG |
| 331 | Mouse CXCL | GACGCCAAGAGCCUGACCGCCUGGAGCCGGACCCUGGUGACCUUC |
| | TAL18-KRAB | AAGGACGUGUUCGUGGACUUCACCCGGGAGGAGUGGAAGCUGCUG |
| | Effector RNA | GACACCGCCCAGCAGAUCCUGUACCGGAACGUGAUGCUGGAGAAC |
| | sequence (from | UACAAGAACCUGGUGAGCCUGGGCUACCAGCUGACCAAGCCCGAC |
| | MR-33722) | GUGAUCCUGCGGCUGGAGAAGGGCGAGGAGCCCUGGCUGGUGGAG |
| | | CGGGAGAUCCACCAGGAGACCCACCCCGACAGCGAGACCGCCUUC |
| | | GAGAUCAAGAGCAGCGUG |
| 334 | Mouse CXCL | GGGAAAUAAGAGAGAAAAGAAGAGUAAGAAGAAAUAUAAGAGCCA |
| | TAL19-KRAB | CCAUGGCCCCAAAGAAGAAGCGGAAAGUCGGAGGUUCAGGCGGCU |
| | mRNA sequence | CGCACAUGGUCGACUUGCGGACUCUGGGAUACUCGCAGCAGCAGC |
| | (MR-33723) | AAGAGAAGAUCAAGCCGAAGGUCCGCUCAACCGUGGCCCAGCACC |
| | | ACGAGGCCCUCGUCGGUCAUGGUUUCACCCAUGCGCACAUCGUCG |
| | | CUCUGAGCCAGCACCCGGCUGCACUUGGAACCGUGGCCGUGAAGU |
| | | ACCAAGACAUGAUUGCCGCGCUGCCUGAAGCAACCCACGAGGCCA |
| | | UUGUGGGAGUGGGGAAGCGGGGAGCCGGAGCCAGGGCCCUCGAAG |
| | | CCCUCCUGACUGUGGCCGGAGAGCUGCGCGGGCCACCACUCCAAC |
| | | UGGACACCGGCCAGCUGCUGAAGAUCGCCAAGCGCGGGGGCGUGA |
| | | CCGCCGUGGAAGCCGUGCAUGCCUGGCGGAACGCCCUGACCGGCG |
| | | CUCCCCUGAAUCUGACUCCGGAACAGGUUGUCGCCAUCGCGAGCC |
| | | AUGAUGGCGGAAAGCAGGCCCUGGAAACGGUGCAGAGACUGUUGC |
| | | CUGUGCUGUGCCAAGCGCACGGACUCACCCCCGAACAGGUCGUGG |
| | | CUAUCGCCUCCAACAUCGGCGGCAAACAGGCACUCGAAACCGUGC |
| | | AGAGGCUCCUGCCCGUGCUGUGUCAGGCCCACGGUCUUACUCCCG |
| | | AACAAGUGGUGGCAAUCGCGUCCAACAACGGCGGAAAACAGGCUC |
| | | UUGAGACAGUGCAGCGCCUGCUGCCUGUGCUCUGCCAAGCCCACG |
| | | GACUGACCCCGGAACAAGUCGUGGCGAUUGCGUCCAGACACGGCG |
| | | GGAAGCAGGCCUUGGAGACUGUGCAGCGGCUGCUGCCCGUGCUUU |
| | | GCCAGGCGCAUGGCCUGACCCCGGAGCAAGUCGUCGCAAUCGCCU |
| | | CCCGCCACGGCGGGAAGCAAGCCUUGGAGACUGUCCAGAGACUCC |
| | | UGCCCGUGCUGUGCCAGGCUCACGGACUUACCCCGGAACAGGUCG |
| | | UGGCUAUCGCCUCAAACAUCGGAGGGAAGCAGGCCCUCGAAACCG |
| | | UGCAGCGGCUUCUGCCUGUGUUGUGCCAGGCCCACGGUCUGACCC |
| | | CUGAACAGGUUGUGGCCAUCGCCUCCAACAUCGGCGGAAAGCAGG |
| | | CACUGGAAACGGUGCAGAGACUGCUCCCCGUGCUAUGCCAAGCGC |
| | | AUGGACUGACUCCGGAACAAGUAGUCGCCAUCGCCAGCAAUGGUG |
| | | GAGGGAAACAAGCCCUGGAGACUGUGCAGCGCCUCCUGCCGGUGC |
| | | UGUGUCAGGCGCACGGACUCACCCCUGAGCAAGUGGUCGCCAUUG |
| | | CCUCGAACGGUGGCGGCAAGCAGGCUCUGGAAACCGUCCAGAGGC |
| | | UGCUCCCGGUGCUCUGCCAAGCCCACGGACUGACCCCAGAACAGG |
| | | UGGUCGCAAUUGCGUCCAACGGCGGUGGAAAACAAGCGCUGGAAA |
| | | CUGUCCAGCGGCUGCUGCCAGUGCUCUGUCAAGCGCACGGCCUGA |
| | | CACCUGAACAAGUCGUGGCAAUCGCGUCCCACGAUGGGGGAAAAC |
| | | AGGCCUUGGAAACGGUCCAGCGCCUGCUGCCCGUGUUGUGUCAGG |
| | | CUCAUGGGCUGACCCCCGAACAAGUGGUGGCCAUUGCCAGCCACG |
| | | ACGGCGGCAAACAGGCCCUUGAGACCGUGCAGCGGUUGCUGCCAG |
| | | UGCUUUGCCAAGCUCACGGGCUGACCCCGGAGCAAGUCGUCGCAA |
| | | UCGCCUCCCACGACGGGGGCAAACAGGCCCUGGAAACCGUGCAGA |
| | | GGCUGCUCCCUGUGUUGUGUCAGGCCCACGGUCUGACUCCUGAAC |
| | | AAGUCGUGGCCAUCGCUUCCAACGGCGGCGGAAAACAGGCACUCG |
| | | AAACUGUGCAGCGCCUGCUGCCCGUGCUGUGCCAGGCUCAUGGAC |
| | | UGACGCCGGAACAGGUUGUCGCCAUCGCCUCGAACAACGGUGGAA |
| | | AACAAGCCCUGGAGACUGUCCAGAGGCUCCUUCCCGUGCUCUGCC |
| | | AAGCACAUGGACUCACCCCGGAACAAGUGGUGGCAAUCGCGUCCC |
| | | GCCACGGAGGAAAGCAGGCCUUGGAAACAGUGCAGCGGCUGCUCC |
| | | CGGUGCUGUGUCAAGCCCACGGACUGACCCCAGAACAGGUCGUGG |
| | | CGAUUGCCAGCAACGGUGGCGGGAAGCAGGCCCUUGAGACUGUGC |
| | | AGAGACUCCUGCCCGUGUUGUGCCAGGCGCAUGGCCUCACCCCUG |
| | | AGCAAGUGGUCGCUAUCGCGAGCCACGAUGGCGGCAGACCCGCCC |
| | | UGGAGUCAAUUGUGGCGCAGCUGUCCCGGCCUGAUCCGGCCCUGG |
| | | CCGCCCUGACCAACGACCACCUGGUGGCCCUCGCCUGCCUGGGCG |
| | | GGCGGCCUGCGCUGGACGCCGUGAAGAAGGGACUGCCCCACGCCC |
| | | CGGCCCUGAUCAAGAGAACCAAUCGGCGCAUUCCCGAGCGGACCU |
| | | CGCACCGCGUGGCAUCUGGGAGCGGAGGAGGAUCCGGCGGAGCCA |
| | | GAGACGACGCCAAGAGCCUGACCGCCUGGAGCCGGACCCUGGUGA |
| | | CCUUCAAGGACGUGUUCGUGGACUUCACCCGGGAGGAGUGGAAGC |
| | | UGCUGGACACCGCCCAGCAGAUCCUGUACCGGAACGUGAUGCUGG |
| | | AGAACUACAAGAACCUGGUGAGCCUGGGCUACCAGCUGACCAAGC |
| | | CCGACGUGAUCCUGCGGCUGGAGAAGGGCGAGGAGCCCUGGCUGG |
| | | UGGAGCGGGAGAUCCACCAGGAGACCCACCCCGACAGCGAGACCG |
| | | CCUUCGAGAUCAAGAGCAGCGUGCCUUCCUCCGGUGGAAAGCGCC |
| | | CAGCGGCUACAAAGAAGGCCGGACAGGCCAAGAAGAAGAAGGGUU |
| | | CGUACCCCUACGACGUGCCUGACUAUGCCUGAGCGGCCGCUUAAU |
| | | UAAGCUGCCUUCUGCGGGGCUUGCCUUCUGGCCAUGCCCUUCUUC |
| | | UCUCCCUUGCACCUGUACCUCUUGGUCUUUGAAUAAAGCCUGAGU |
| | | AGGAAGUCUAGAAAAAAAAAAAAAAAAAAAAAAAAAAAAAAAAAA |
| | | AAAAAAAAAAAAAAAAAAAAAAAAAAAAAAAAAAAAAAAAAAAAA |
| | | AAAAAAAAAAAAAAAAAAAAAAAAAAAAAAAA |
| 336 | Mouse CXCL | CACAUGGUCGACUUGCGGACUCUGGGAUACUCGCAGCAGCAGCAA |
| | TAL19-KRAB | GAGAAGAUCAAGCCGAAGGUCCGCUCAACCGUGGCCCAGCACCAC |
| | DBD RNA | GAGGCCCUCGUCGGUCAUGGUUUCACCCAUGCGCACAUCGUCGCU |
| | sequence (from | CUGAGCCAGCACCCGGCUGCACUUGGAACCGUGGCCGUGAAGUAC |
| | MR-33723) | CAAGACAUGAUUGCCGCGCUGCCUGAAGCAACCCACGAGGCCAUU |
| | | GUGGGAGUGGGGAAGCGGGGAGCCGGAGCCAGGGCCCUCGAAGCC |
| | | CUCCUGACUGUGGCCGGAGAGCUGCGCGGGCCACCACUCCAACUG |
| | | GACACCGGCCAGCUGCUGAAGAUCGCCAAGCGCGGGGGCGUGACC |
| | | GCCGUGGAAGCCGUGCAUGCCUGGCGGAACGCCCUGACCGGCGCU |
| | | CCCCUGAAUCUGACUCCGGAACAGGUUGUCGCCAUCGCGAGCCAU |
| | | GAUGGCGGAAAGCAGGCCCUGGAAACGGUGCAGAGACUGUUGCCU |
| | | GUGCUGUGCCAAGCGCACGGACUCACCCCCGAACAGGUCGUGGCU |
| | | AUCGCCUCCAACAUCGGCGGCAAACAGGCACUCGAAACCGUGCAG |
| | | AGGCUCCUGCCCGUGCUGUGUCAGGCCCACGGUCUUACUCCCGAA |
| | | CAAGUGGUGGCAAUCGCGUCCAACAACGGCGGAAAACAGGCUCUU |
| | | GAGACAGUGCAGCGCCUGCUGCCUGUGCUCUGCCAAGCCCACGGA |
| | | CUGACCCCGGAACAAGUCGUGGCGAUUGCGUCCAGACACGGCGGG |
| | | AAGCAGGCCUUGGAGACUGUGCAGCGGCUGCUGCCCGUGCUUUGC |
| | | CAGGCGCAUGGCCUGACCCCGGAGCAAGUCGUCGCAAUCGCCUCC |
| | | CGCCACGGCGGGAAGCAAGCCUUGGAGACUGUCCAGAGACUCCUG |
| | | CCCGUGCUGUGCCAGGCUCACGGACUUACCCCGGAACAGGUCGUG |
| | | GCUAUCGCCUCAAACAUCGGAGGGAAGCAGGCCCUCGAAACCGUG |
| | | CAGCGGCUUCUGCCUGUGUUGUGCCAGGCCCACGGUCUGACCCCU |
| | | GAACAGGUUGUGGCCAUCGCCUCCAACAUCGGCGGAAAGCAGGCA |
| | | CUGGAAACGGUGCAGAGACUGCUCCCCGUGCUAUGCCAAGCGCAU |
| | | GGACUGACUCCGGAACAAGUAGUCGCCAUCGCCAGCAAUGGUGGA |
| | | GGGAAACAAGCCCUGGAGACUGUGCAGCGCCUCCUGCCGGUGCUG |
| | | UGUCAGGCGCACGGACUCACCCCUGAGCAAGUGGUCGCCAUUGCC |
| | | UCGAACGGUGGCGGCAAGCAGGCUCUGGAAACCGUCCAGAGGCUG |
| | | CUCCCGGUGCUCUGCCAAGCCCACGGACUGACCCCAGAACAGGUG |
| | | GUCGCAAUUGCGUCCAACGGCGGUGGAAAACAAGCGCUGGAAACU |
| | | GUCCAGCGGCUGCUGCCAGUGCUCUGUCAAGCGCACGGCCUGACA |
| | | CCUGAACAAGUCGUGGCAAUCGCGUCCCACGAUGGGGGAAAACAG |
| | | GCCUUGGAAACGGUCCAGCGCCUGCUGCCCGUGUUGUGUCAGGCU |
| | | CAUGGGCUGACCCCCGAACAAGUGGUGGCCAUUGCCAGCCACGAC |
| | | GGCGGCAAACAGGCCCUUGAGACCGUGCAGCGGUUGCUGCCAGUG |
| | | CUUUGCCAAGCUCACGGGCUGACCCCGGAGCAAGUCGUCGCAAUC |
| | | GCCUCCCACGACGGGGGCAAACAGGCCCUGGAAACCGUGCAGAGG |
| | | CUGCUCCCUGUGUUGUGUCAGGCCCACGGUCUGACUCCUGAACAA |
| | | GUCGUGGCCAUCGCUUCCAACGGCGGCGGAAAACAGGCACUCGAA |
| | | ACUGUGCAGCGCCUGCUGCCCGUGCUGUGCCAGGCUCAUGGACUG |
| | | ACGCCGGAACAGGUUGUCGCCAUCGCCUCGAACAACGGUGGAAAA |
| | | CAAGCCCUGGAGACUGUCCAGAGGCUCCUUCCCGUGCUCUGCCAA |
| | | GCACAUGGACUCACCCCGGAACAAGUGGUGGCAAUCGCGUCCCGC |
| | | CACGGAGGAAAGCAGGCCUUGGAAACAGUGCAGCGGCUGCUCCCG |
| | | GUGCUGUGUCAAGCCCACGGACUGACCCCAGAACAGGUCGUGGCG |
| | | AUUGCCAGCAACGGUGGCGGGAAGCAGGCCCUUGAGACUGUGCAG |
| | | AGACUCCUGCCCGUGUUGUGCCAGGCGCAUGGCCUCACCCCUGAG |
| | | CAAGUGGUCGCUAUCGCGAGCCACGAUGGCGGCAGACCCGCCCUG |
| | | GAGUCAAUUGUGGCGCAGCUGUCCCGGCCUGAUCCGGCCCUGGCC |
| | | GCCCUGACCAACGACCACCUGGUGGCCCUCGCCUGCCUGGGCGGG |
| | | CGGCCUGCGCUGGACGCCGUGAAGAAGGGACUGCCCCACGCCCCG |
| | | GCCCUGAUCAAGAGAACCAAUCGGCGCAUUCCCGAGCGGACCUCG |
| | | CACCGCGUG |
| 338 | Mouse CXCL | GACGCCAAGAGCCUGACCGCCUGGAGCCGGACCCUGGUGACCUUC |
| | TAL19-KRAB | AAGGACGUGUUCGUGGACUUCACCCGGGAGGAGUGGAAGCUGCUG |
| | Effector RNA | GACACCGCCCAGCAGAUCCUGUACCGGAACGUGAUGCUGGAGAAC |
| | sequence (from | UACAAGAACCUGGUGAGCCUGGGCUACCAGCUGACCAAGCCCGAC |
| | MR-33723) | GUGAUCCUGCGGCUGGAGAAGGGCGAGGAGCCCUGGCUGGUGGAG |
| | | CGGGAGAUCCACCAGGAGACCCACCCCGACAGCGAGACCGCCUUC |
| | | GAGAUCAAGAGCAGCGUG |

TABLE 36
Exemplary TAL domain mouse target sequences, e.g., for an
expression repressor comprising an effector moiety, e.g., KRAB

| SEQ ID NO | Description | Sequence |
|---|---|---|
| 318 | Mouse TAL16-KRAB Target Sequence | GTCTAATCCTTGGGAGTG |
| | (MR-32720) | GRCm38: chr5:90891101- |
| | | 90891118 |
| 325 | Mouse TAL17-KRAB Target Sequence | CTGTAGTACACCAAGTGG |
| | (MR-32721) | GRCm38: chr5:90890903- |
| | | 90890920 |
| 332 | Mouse TAL17-KRAB Target Sequence | GTTAGGTCTCCACTGATG |
| | (MR-32722) | GRCm38: chr5:90903571- |
| | | 90903588 |
| 339 | Mouse TAL17-KRAB Target Sequence | CAGGGAATTTCCCTGGTC |
| | (MR-32723) | GRCm38: chr5:90903800- |
| | | 90903817 |

### Example 43: Bicistronic Expression Repressor Downregulates IL-8 mRNA and Protein in Multiple Tumor Cell Lines

This example describes an experiment demonstrating decreased expression of IL8 in A549 Cells (ATCCR CCL-185), SKHTEP1 Cells (ATCC® HTB-52), H2009 Cells (ATCC® CRL-5911), and MDA-MB-231 Cells (ATCC® HTB-26). Cells were cultured and plated as follows: SKHEP1 in EMEM (ATCC-30-2003), H2009 in RPMI 1640 (GIBCO 11-875-119) A549 and MDA-MB-231 in DMEM (GIBCO 11-995-065). Complete media was made with 10% FBS (Atlanta Biologicals cat #S11550).

All cells were transfected with LNPs containing bicistronic zinc finger-TAL mRNA (MR-32905) which were added to the media at a final concentration of 1 μg/ml in SSOP lipid mix.

After completion of 48 hr incubation, TNF-α (Sigma cat #T6674 10 g) was added at 10 ng/ml final concentration and incubated for 2 hours.

After completion of the 2 hr incubation with TNF-α, RNA was isolated using the Macherey-Nagel Inc RNA extraction kit (cat #740466.4) following the Manufacturer's protocol. RNA samples were retrotranscribed to cDNA using LunaScript RT SuperMix Kit (New England Biolabs) and analyzed by quantitative PCR using individual specific Taqman primer/probe sets with the Taqman Fast Advanced Master Mix (Thermo Scientific).

Gene expression was quantified relative to the human GAPDH reference gene using the ΔΔCt method. Changes in gene expression were further quantified by measuring IL8 gene expression in expression repressor treated cells as a percent of the IL8 gene expression in cells treated with only TNF-α.

Cell supernatant was collected after the 2 hr TNF-α incubation and used for ELISA. The Abcam IL8 ELISA (AB214030) protocol was followed in order to quantify IL8 protein levels from cell supernatant.

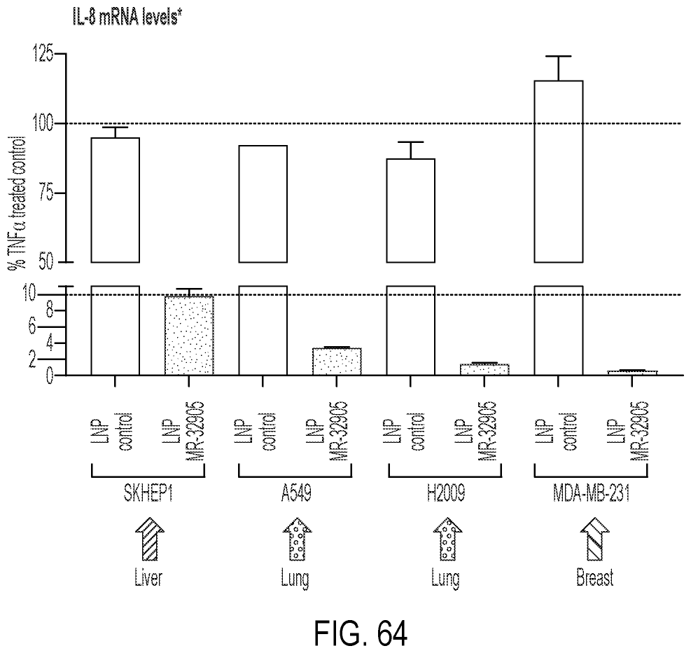

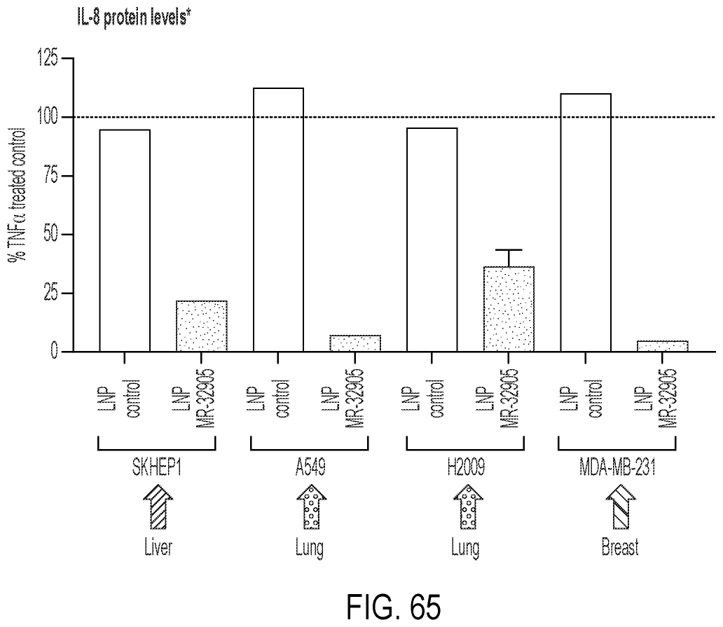

The results show targeting the E1 enhancer and IL8 promoter region using a bicistronic zinc finger-TAL mRNA (MR-32905) can be used to decrease IL8 expression and IL8 protein levels in cancer cell lines, such as the 4 cancer cell lines mentioned above, and that expression is decreased at 48 hours post-treatment (FIG. 64 and FIG. 65).

### Example 44: Bicistronic Expression Repressor Downregulates CXCL1 mRNA in Multiple Tumor Cell Lines

This example describes an experiment demonstrating decreased expression of CXCL1 in SKHEP1 cells, A2549 cells, H2009 Cells (ATCC® CRL-5911) and MDA-MB-231 Cells (ATCC® HTB-26). Cells were cultured and plated as follows: H2009 RPMI 1640 (GIBCO 11-875-119) MDA-MB-231 in DMEM (GIBCO 11-995-065). Complete media was made with 10% FBS (Atlanta Biologicals cat #S11550).

All cells were transfected with LNPs containing zinc finger-TAL mRNA (MR-32905) which were added to the media at a final concentration of 1 μg/ml SSOP lipid mix.

After completion of 48 hr incubation, TNF-α (Sigma cat #T6674 10 μg) was added at 10 ng/ml final concentration and incubated for 2 hours.

After completion of the 2 hr incubation with TNF-α, RNA was isolated using the Macherey-Nagel Inc RNA extraction kit (cat #740466.4) following the Manufacturer's protocol. RNA samples were retrotranscribed to cDNA using LunaScript RT SuperMix Kit (New England Biolabs) and analyzed by quantitative PCR using individual specific Taqman primer/probe sets with the Taqman Fast Advanced Master Mix (Thermo Scientific).

Gene expression was quantified relative to the human GAPDH reference gene using the ΔΔCt method. Changes in gene expression were further quantified by measuring CXCL1 gene expression in expression repressor treated cells as a percent of the CXCL1 gene expression in cells treated with only TNF-α.

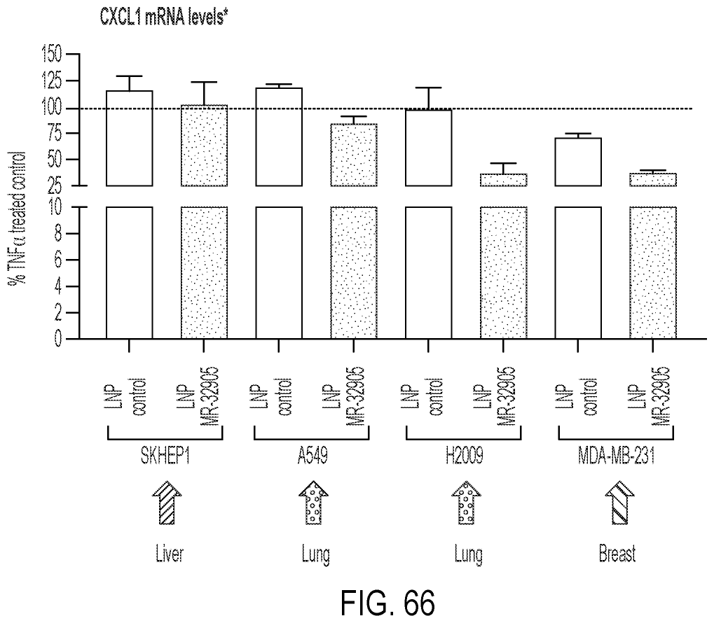

The results show targeting the E1 enhancer and IL8 promoter region using a bicistronic zinc finger-TAL mRNA (MR-32905) can be used to decrease expression of CXCL1 in cancer cell lines, such as the cancer cell lines mentioned above, and that expression is decreased at 48 hours post-treatment (FIG. 66).

### Example 45: Bicistronic Expression Repressor Downregulates Endogenous IL-8 Expression in a Breast Cancer Cell Line

This example describes an experiment demonstrating decreased expression of IL-8 in MDA-MB-231 Cells (ATCC® HTB-26). Cells were cultured and plated as follows: MDA-MB-231 in DMEM (GIBCO 11-995-065). Complete media was made with 10% FBS (Atlanta Biologicals cat #S11550).

All cells were transfected with LNPs containing zinc finger-TAL mRNA (MR-32905) which were added to the media at a final concentration of 1 μg/ml SSOP lipid mix.

Cells were not stimulated with TNF-α in order to measure the ability of the expression repressor to down regulate endogenous IL8 levels.

After 48 hr incubation, RNA was isolated using the Macherey-Nagel Inc RNA extraction kit (cat #740466.4) following the Manufacturer's protocol. RNA samples were retrotranscribed to cDNA using LunaScript RT SuperMix Kit (New England Biolabs) and analyzed by quantitative PCR using individual specific Taqman primer/probe sets with the Taqman Fast Advanced Master Mix (Thermo Scientific).

Gene expression was quantified relative to the human GAPDH reference gene using the ΔΔCt method. Changes in gene expression were further quantified by measuring IL8 gene expression in expression repressor treated cells as a percent of untreated cells.

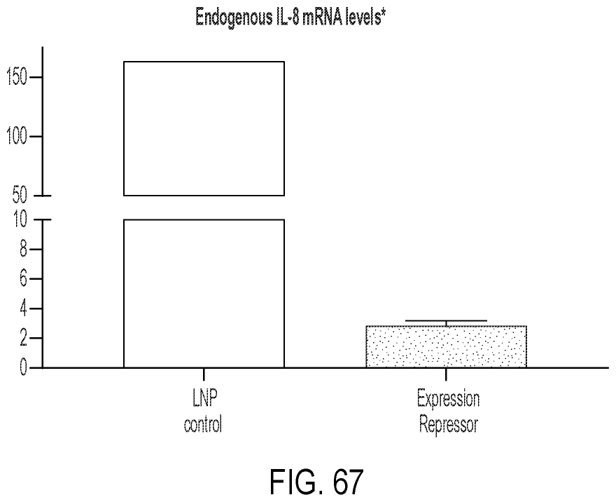

The results show targeting the E1 enhancer and IL8 promoter region using a bicistronic zinc finger-TAL mRNA (MR-32905) can be used to decrease endogenous IL8 expression in MDA-MB-231 cells and that expression is decreased at 48 hours post-treatment (FIG. 67).

### Example 46: Antitumor Activity of Bicistronic Expression Repressor in A549 NSCLC Xenograft Model

This example demonstrates the antitumor activity of bicistronic expression repressor (MR-32905) in NSCLC xenograft model. NSCLC cells (A549 cell line, adenocarcinoma alveolar epithelial cells) were subcutaneously implanted to produce the xenograft mouse model. Disease was induced in ninety female nude mice by inoculating them subcutaneously in the left flank with 1×10⁷ A549 cells. Treatment was initiated when the tumors reached a mean volume of 156.0 mm³ (standard deviation±35.5 mm³, CV=22.8%, range 74.1-261.6 mm³). Mice were allocated to five (5) groups often (10) mice such that mean tumor volume in each group was within the range of 150.5 to 159.4 mm³. PBS (control), cisplatin, GFP control, and expression repressors were given via intra-venous injection (IV) as described in Table 39 at doses equivalent to the total drug product. Animal weights and conditions were recorded daily, and tumors were measured using digital calipers on Mondays, Wednesdays and Fridays.

TABLE 39
Study design

| | | | | Route of | |
|---|---|---|---|---|---|
| Group | Number of | | Treatment | Adminis- | Dose |
| Number | Animals | Treatment | Schedule | tration | (mg/kg) |
| 1 | 10 females | PBS | Q5d × 4 | IV | N/A |
| 2 | 10 females | GFP control | Q5d × 4 | IV | GFP 3 |
| | | | | | mg/kg |
| 3 | 10 females | MR-32905 | Q5d × 4/5 | IV | 1 mg/kg |
| 4 | 10 females | MR-32905 | Q5d × 4/5 | IV | 3 mg/kg |
| 5 | 10 females | Cisplatin | Q15d | IV | 1 mg/kg |

MR-32905 was formulated in MC3 LNP. Dosing of LNP formulated bicistronic expression repressor (MR-32905) was administered at 1 mg/kg Q5D and 3 mg/kg Q5D via tail vein. PBS and cisplatin (1 mg/kg) were used as control.

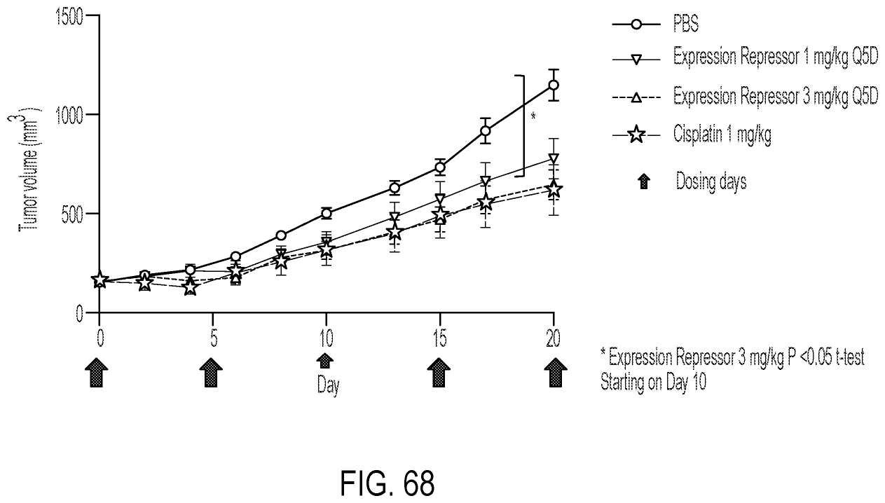

The results show a decrease in tumor volume (mm³) after administration of the expression repressor (FIG. 68).

### Example 47: Further Antitumor Activity of Bicistronic Expression Repressor in A549 NSCLC Xenograft Model

This example demonstrates the antitumor activity of bicistronic expression repressor in an NSCLC xenograft model. Tumor bearing mice were administered GFP mRNA control at 3 mg/kg every five days for a total of 4 doses. Bicistronic expression repressor (MR-32905) was administered at 1 mg/kg or 3 mg/kg every five days for a total of 4 doses. Cisplatin (Accord Healthcare) was administered at 1 mg/kg once every 15 days. All treatments were administered by way of intra-venous injection. No animal deaths were observed during this study.

The group treated with bicistronic expression repressor (MR-32905) at 3 mg/kg had a statistically significant reduction in weight gain relative to the PBS group (p=0.0011). The group treated with bicistronic expression repressor (MR-32905) at 1 mg/kg showed statistically significant reductions in tumor growth relative to the PBS group (p=0.0170). The group treated with bicistronic expression repressor (MR-32905) at 3 mg/kg showed statistically significant reductions in tumor growth relative to the PBS group (p=0.0003). The group treated with GFP control 1 mg/kg did not show statistically significant reductions in tumor growth relative to the PBS group (p=0.9649). The group treated with cisplatin 1 mg/kg showed statistically significant reductions in tumor growth relative to the PBS group (p<0.0001).

Disease was induced in fifty (50) female nude mice by inoculating them subcutaneously in the left flank with 1×10⁷ A549 cells. Treatment was initiated when the tumors reached a mean volume of 155.4 mm³ (standard deviation±39.8 mm³, CV=25.6%, range 74.1-249.9 mm³). Mice were allocated to five (5) groups of ten (10) mice, such that mean tumor volume in each group was within the range of 150.5 to 159.4 mm³. PBS, cisplatin, GFP control, and bicistronic expression repressor (MR-32905) were administered by way of intra-venous injection (IV) as shown in Table 40. Control GFP (mRNA) and bicistronic expression repressor (mRNA MR-32905; SEQ ID NO: 301) were formulated in MC3 LNP (9% DOPC, 45% MC3, 44% Cholesterol, 2% PEG2K-DMB). Animal weights and conditions were recorded daily, and tumors were measured on Mondays, Wednesdays and Fridays.

TABLE 40
Study Design

| Group | Number of | | Treatment | | Dose |
|---|---|---|---|---|---|
| Number | Animals | Treatment | Schedule | RoA | (mg/kg) |
| 1 | 10 females | PBS | Q5d × 4 | IV | N/A |
| 6 | 10 females | GFP control | Q5d × 4 | IV | 3 mg/kg |
| 7 | 10 females | MR-32905 | Q5d × 4/5 | IV | 1 mg/kg |
| 8 | 10 females | MR-32905 | Q5d × 4/5 | IV | 3 mg/kg |
| 9 | 10 females | Cisplatin | Q15d | IV | 1 mg/kg |

### Animals, Randomization, Housing and Diet

Fifty (50) female nude mice (Jackson Labs 007850) aged 6 to 7 weeks were used. The mean body weight prior to treatment was 20.4 grams (SD±1.5 g, range 18.2-24.1 g). Animals were individually numbered and housed in groups of 5 animals per cage. Animals were acclimatized for three days prior to study tumor cell implantation. Mice were divided into five (5) groups prior to the initiation of treatment based on tumor volume. Animals were housed in ventilated cage racks providing HEPA-filtered air, which were housed in animal rooms at a constant temperature of 70° F.+/−2° F. Relative humidity was monitored but not actively controlled. The relative humidity in the animal housing rooms during this study was between 30% and 70%. A light/dark cycle of 12 hours on and 12 hours off was maintained. Bedding was changed a minimum of once per week. Animals were fed with sterile Envigo 2920X sterile rodent chow and sterile water was provided at all times.

### Experimental Procedures

### Tissue Culture

A549 cells (ATCC #CRL-5911) were grown in DMEM medium supplemented with 10% fetal bovine serum, 1% penicillin and streptomycin. Cells were sub-cultured by dilution at a ratio of 1:4.

### Tumor Implantation

A549 cells were harvested by centrifugation and counted using a hemocytometer. Cells were resuspended in PBS at a 1×10⁸ cells per mL. Cells were placed on ice and mixed with an equal volume of Matrigel (Corning CB-40234). This mixture was kept on ice and injected into the left flank of mice in a volume of 0.2 mL, equivalent to 1×10⁷ cells per mouse.

### Weights and Survival

All animals were weighed every day in order to assess possible differences in animal weight among treatment groups as an indication of possible toxicity resulting from the treatments.

### Evaluation of Results

Statistical differences between treatment groups were determined using Mann-Whitney Rank Sum or ANOVA tests with a critical value of 0.05.

### Results

No deaths were seen during this study.

### Animal Weights

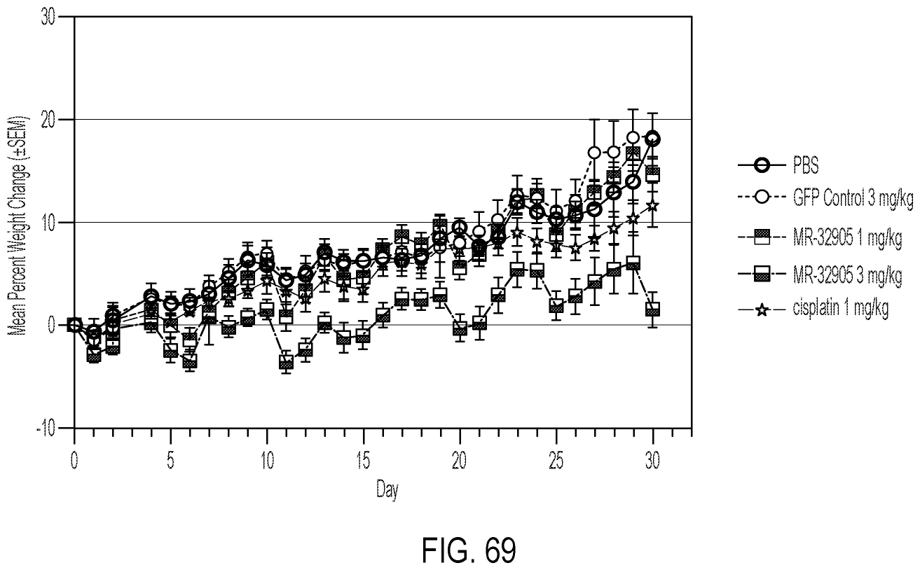

Mean percentage weight changes by day for each treatment group are shown in FIG. 69. All groups in this study had mean weight gains over the study time course. Mice treated with PBS (Group 1) had a mean weight gain of 10.7% on Day 27. Mice treated with GFP control at 3 mg/kg (Group 6) had a mean weight gain of 12.1% on Day 27. Mice treated with bicistronic expression repressor (MR-32905) at 1 mg/kg (Group 7) had a mean weight gain of 11.0% on Day 27. Mice treated with bicistronic expression repressor (MR-32905) at 3 mg/kg (Group 8) had a mean weight gain of 2.8% on Day 27. Mice treated with Cisplatin at 1 mg/kg (Group 9) had a mean weight gain of 7.5% on Day 27.

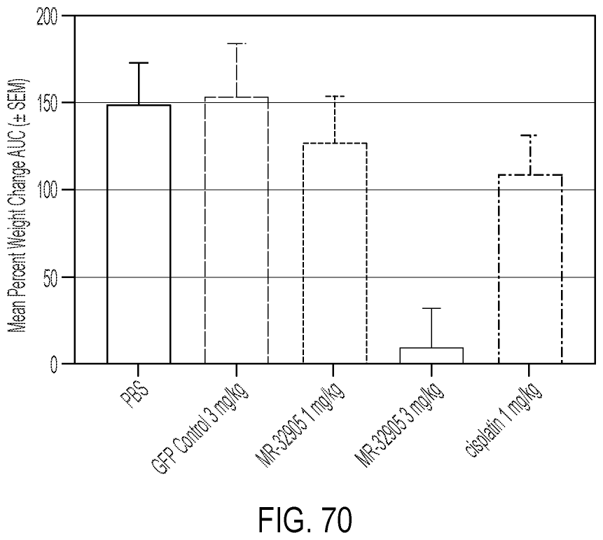

To evaluate the significance of the differences seen in weight gain the Area Under the Curve (AUC) for the percentage weight change for each animal was calculated and the groups compared using a one-way ANOVA test on weight changes to Day 27. A statistically significant difference was seen between the group treated with bicistronic expression repressor (MR-32905) at 3 mg/kg and the PBS group (p=0.0011). These data are shown in FIG. 70.

### Tumor Volumes

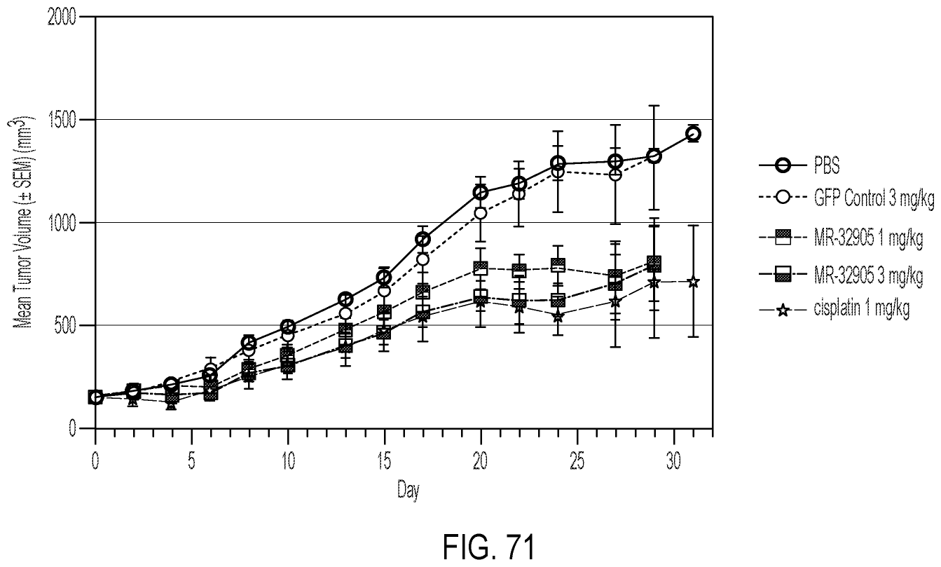

The data for tumor volume change during the study are shown in FIG. 71. The mean tumor volume for the PBS control group (Group 1) increased from 152.7 mm³ on Day 0 to 1291.5 mm³ on Day 24. Mice treated with GFP control at 3 mg/kg (Group 6) had a mean tumor volume of 150.5 mm³ on Day 0, which increased to 1246.2 mm³ on Day 24. Mice treated with bicistronic expression repressor (MR-32905) at 1 mg/kg (Group 7) had a mean tumor volume of 159.3 mm³ on Day 0, which increased to 792.6 mm³ on Day 24. Mice treated with bicistronic expression repressor (MR-32905) at 3 mg/kg (Group 8) had a mean tumor volume of 157.0 mm³ on Day 1, which increased to 642.8 mm³ on Day 24. Mice treated with cisplatin at 1 mg/kg (Group 9) had a mean tumor volume of 157.4 mm³ on Day 1, which increased to 545.3 mm³ on Day 24.

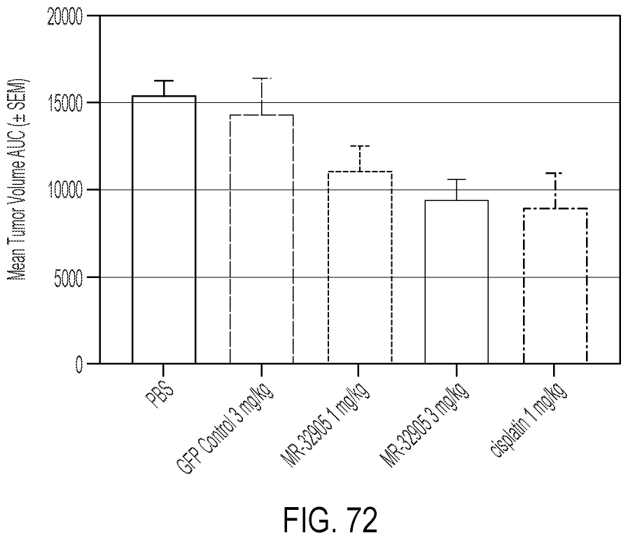

Additional analyses of the tumor volume data were performed by calculating the mean area under the curve (AUC) for each tumor and comparing groups using a one-way ANOVA test. This analysis indicated that there were statistically significant differences between the PBS control group and the groups treated with bicistronic expression repressor (MR-32905) at 3 mg/kg (p=0.0216), and cisplatin (p=0.0124). The differences between the groups treated with bicistronic expression repressor (MR-32905) at 1 mg/kg or GFP control at 1 mg/kg and the PBS control were not statistically significant. These data are shown in FIG. 72.

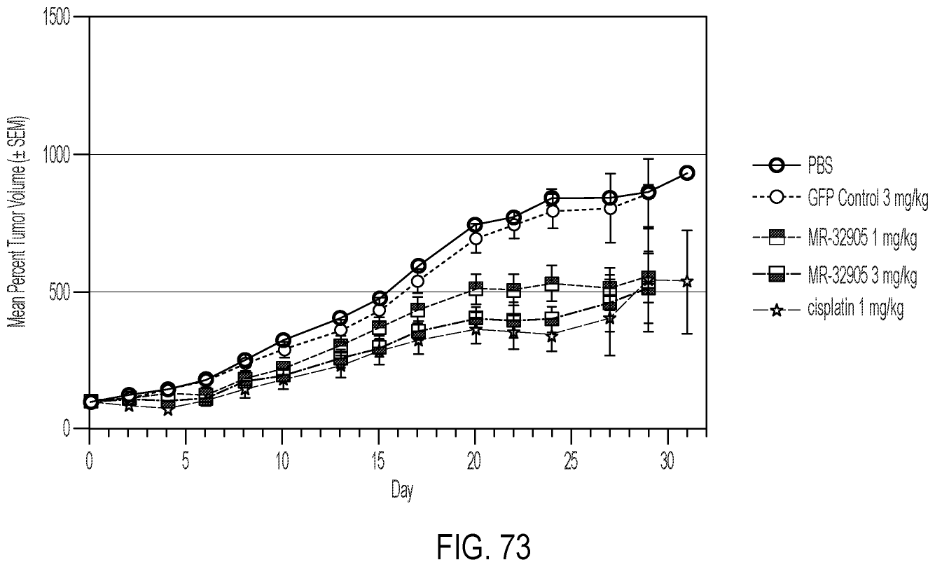

The changes in percent tumor volume during the course of the study are shown in FIG. 73. The mean percent tumor volume for the PBS control group (Group 1) increased to 840.5% of the starting volume by Day 24. The mean percent tumor volume for the group treated with GFP control at 3 mg/kg (Group 6) increased to 797.3% of starting volume on Day 24. The mean percent tumor volume for the group treated with bicistronic expression repressor (MR-32905) at 1 mg/kg (Group 7) increased to 530.6% of starting volume on Day 24. The mean percent tumor volume for the group treated with bicistronic expression repressor (MR-32905) at 3 mg/kg (Group 8) increased to 400.1% of starting volume on Day 24. The mean percent tumor volume for the group treated with cisplatin at 1 mg/kg (Group 9) increased to 338.4% of starting volume on Day 24.

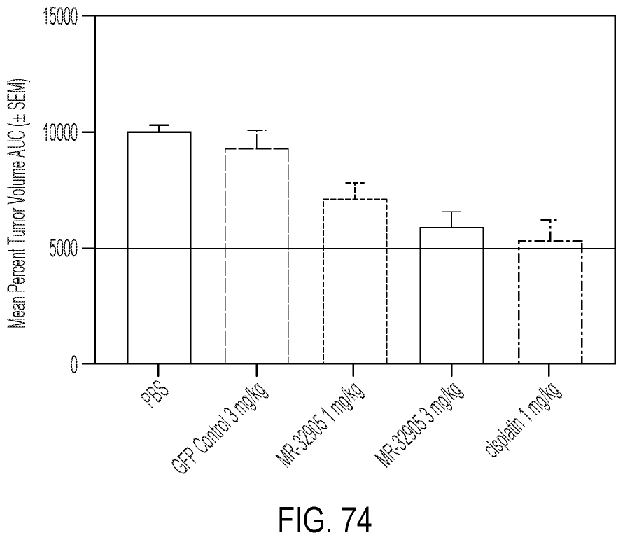

Additional analyses of the percent tumor volume data were performed by calculating the mean area under the curve (AUC) for each tumor and comparing groups using a one-way ANOVA test. This analysis indicated that there were statistically significant differences between the PBS control group and the groups treated with bicistronic expression repressor (MR-32905) at 1 mg/kg (p=0.0170), bicistronic expression repressor (MR-32905) at 3 mg/kg (p=0.0003), and cisplatin at 1 mg/kg (p<0.0001). The differences between the groups treated with GFP control and the PBS control were not statistically significant. These data are shown in FIG. 74.

These data show the efficacy of bicistronic expression repressors of this disclosure. No animal deaths were observed during this study. All groups in this study had mean weights gains during this study. A statistically significant reduction in weight gain was seen in the group treated with bicistronic expression repressor (MR-32905) at 3 mg/kg (p=0.0011). The group treated with GFP control at 1 mg/kg did not show statistically significant reductions in tumor growth relative to the PBS group (p=0.9649). The group treated with cisplatin at 1 mg/kg showed statistically significant reductions in tumor growth relative to the PBS group (p<0.0001).

### Example 48: Efficacy of Expression Repressors Against a Model for Acute Respiratory Distress Syndrome (ARDS)

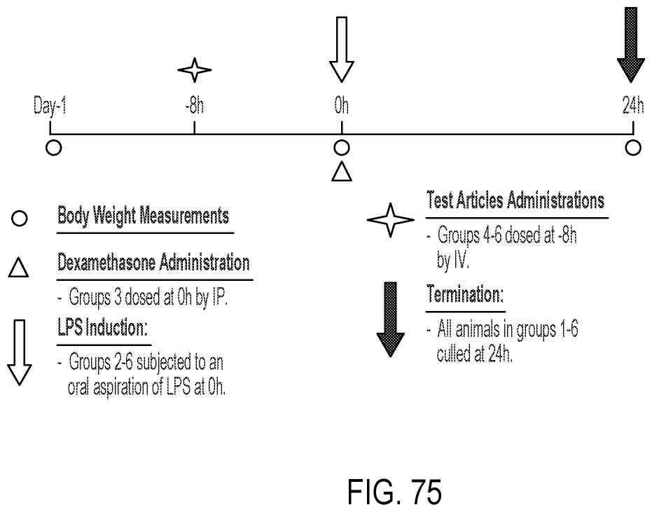

This example demonstrates the efficacy of expression repressors described herein against ARDS. The experimental design is shown in FIG. 75.

A hallmark feature of asthma is airway obstruction and is characterized by the recruitment of inflammatory cells, bronchial hyperreactivity, mucus production, and airway remodeling and narrowing. Bronchoalveolar lavage fluid (BALF) analysis has proved remarkably successful in helping to elucidate the airway pathology of asthma and the action of anti-inflammatory drugs on the airway. To evaluate the dynamic changes in immune responses under homeostatic and disease states upon lipopolysaccharide (LPS) challenge, immunophenotyping with multicolor flow cytometry panels and absolute count for the identification of novel and established immune cell types in both BALF and peripheral blood samples was performed. Identification and quantification of pulmonary myeloid cell subsets in conjunction with a lymphocyte phenotyping panel provided a complete characterization of pulmonary immune cell composition in this study. Here, the complete characterization of pulmonary immune cell composition by defining the relative frequencies of all major leukocyte subsets in human blood, BALF, in both normal and diseased lung tissues, is described.

Cellularity analysis in BALF shows that LPS treatment reduced mouse alveolar macrophage and enhanced neutrophils and B Cells recruitment in the lung. The acute lung injury (ALI) model has shown that LPS suppresses alveolar macrophage and induces neutrophil in both cell number and frequency. Administration of Treatment-2 (lmpk) significantly repressed neutrophil recruitment in BALF when compared to disease only group. Cellularity analysis in blood cells shows that LPS treatment reduced mouse T and B cells and enhanced monocytes and neutrophils population in the peripheral circulation system. As shown in the figures, significant enhancement of blood monocyte frequency and reduced blood B cells frequency was observed.

In summary, the present findings demonstrate that Treatment 2 at Impk may provide beneficial efficacy in the treatment and/or prevention of acute inflammation associated with the accumulation of neutrophils in the respiratory tract, in particular, in acute respiratory distress syndrome (ARDS).

Murine LPS lung inflammation model was used to study acute inflammation in the lungs. To induce acute respiratory distress syndrome (ARDS). Each mouse received 50 L of LPS administered by oropharyngeal aspiration (O.A.) in groups 2-6 at 0 h. Mice were anesthetized with isoflurane/oxygen and suspended by cranial incisors on a thin rubber band from an angled stand. The tongue was gently extracted from the mouth using blunt forceps to visualize the base of the tongue and the pharynx. The LPS/saline suspension was placed on the posterior pharynx. Respiration was monitored to ensure the suspension is fully aspirated before the tongue is released. Mice in Group 1 (naïve control) received no disease induction, and no treatment. Group 2 mice (disease only control) only received 50 L LPS. Group 3 was administered 10 mg/kg Dexamethasone at 0 h by IP. Groups 4-6 were administered the respective treatment at −8 h by IV. The treatment is further described in Tables 41-43, below.

TABLE 41
Study Design

| Group | Group | Group | Disease | | Dose | Dosing | | Dosing | |
|---|---|---|---|---|---|---|---|---|---|
| # | Size | Description | Induction | Route | (mg/kg) | volume | | Regime | Termination |
| 1 | N = 10 | Naive | NA | NA | NA | NA | | NA | 24 h post |
| 2 | N = 10 | Disease Only | 50 μL of LPS | NA | NA | NA | | NA | LPS |
| 3 | N = 10 | Dexamethasone | administered | IP | 10 | 10 | mL/kg | 0 H | induction |
| 4 | N = 10 | Treatment-1 | via OA at 0 h | IV | 1 | 100 | μL | −8 H | |
| | | 1mpk | | | | | | | |
| 5 | N = 10 | Treatment-2 | | | 1 | 100 | μL | | |
| | | 1mpk | | | | | | | |
| 6 | N = 10 | Treatment-3 | | | 1 | 100 | μL | | |
| | | 1mpk | | | | | | | |

TABLE 42
mRNA

| ID# | SEQ ID NO | Description |
|---|---|---|
| GD-33455 | 371 | El targeting guide |
| MR-28122 | 205 | dCas9-KRAB |
| MR-33721 | 320 | TAL17-KRAB (CXCL1 Promoter) |
| MR-33722 | 327 | TAL18-KRAB (CXCL2 Promoter) |
| MR-28991 | | GFP RNA |

TABLE 43
Treatments

| | | | In vitro formulation |
|---|---|---|---|
| | | | Ratio of |
| Group# | Treatment | Description | Guide:Effector:TAL |
| 4 | Treatment-1 | MR-28991; GFP Negative | GFP Only |
| | | Control | |
| 5 | Treatment-2 | GD-33455 + MR-28122 + | 1:1:1 |
| | | MR-33721 | |
| 6 | Treatment-3 | GD-33455 + MR-28122 + | 1:1:1 |
| | | MR-33722 | |

### CONCLUSIONS

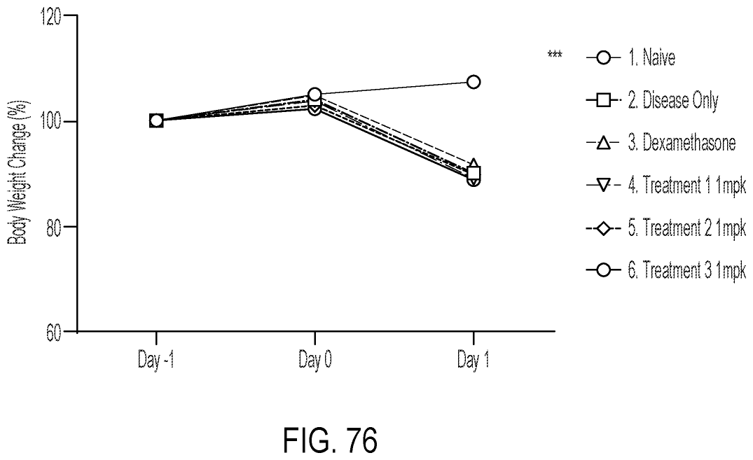

Body Weight: Body weight measurements were performed 1× daily until termination. Body weight change for each animal was calculated in comparison to the initial body weight on day −1. LPS administration caused body weight loss during the study. The treatments showed no effect on body weight loss. These data are presented in FIG. 76.

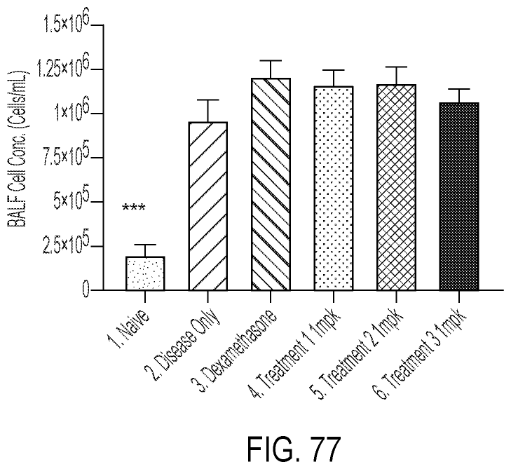

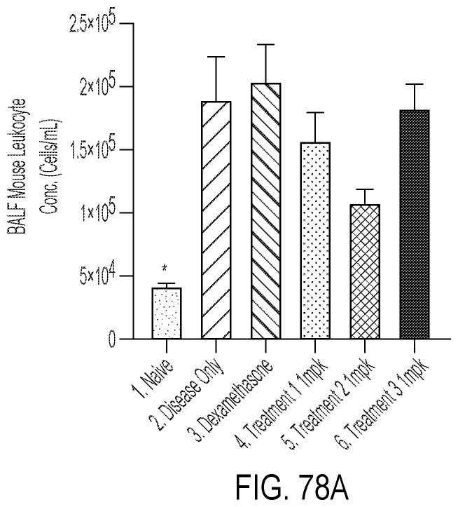

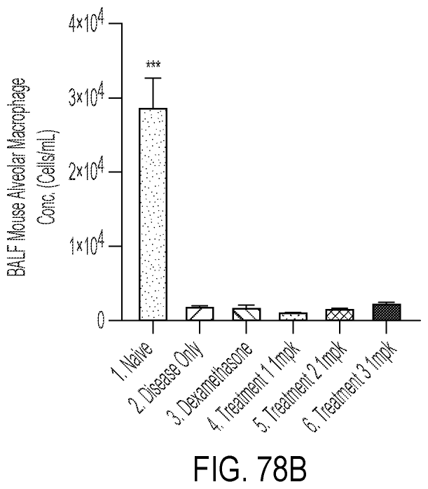

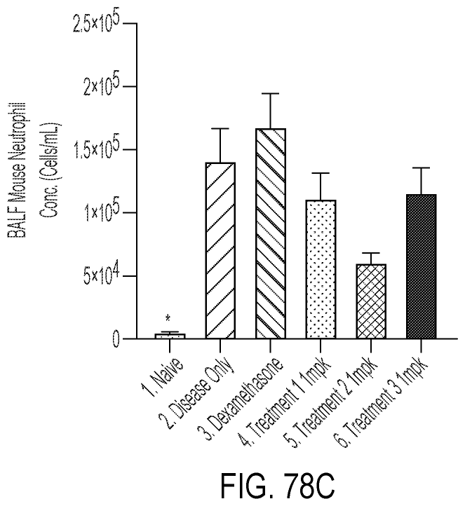

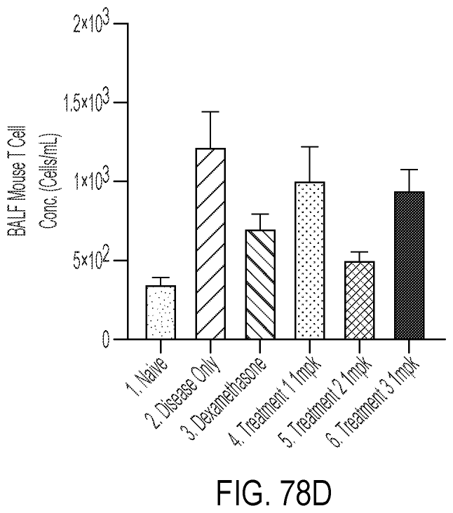

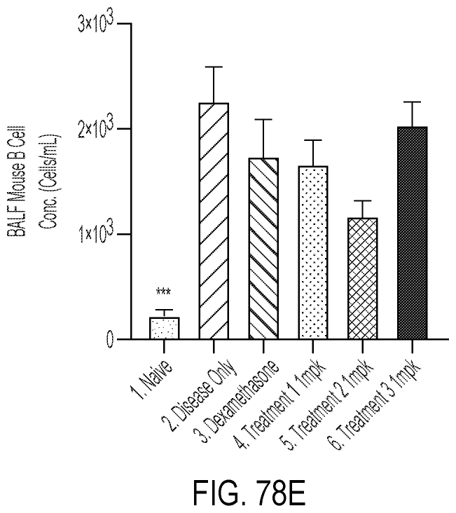

BALF [Cell]: BALF cells concentration was determined after BALF collection (FIGS. 77 and 78A-78E, Table 44). LPS administration significantly increase BALF cell numbers (FIG. 77); however, none of the expression repressors showed significant BALF cell reduction when compared to disease only group.

TABLE 44
Average cells/mL BALF

| Group | CD45+ cells | AM | Neu | T-cells | B-cells |
|---|---|---|---|---|---|
| 2. Disease Only | 187963.4 | 1744.78 | 139259 | 1209.59 | 2239.21 |
| 4. Treatment-1 | 155410.8 | 968.56 | 109660 | 993.749 | 1641.43 |
| at 1 mpk | | | | | |
| 5. Treatment-2 | 106301.3 | 1446.42 | 58587 | 490.026 | 1150.37 |
| at 1 mpk | | | | | |
| 6. Treatment-3 | 180968.5 | 2190.26 | 114110 | 932.247 | 2016.47 |
| at 1 mpk | | | | | |

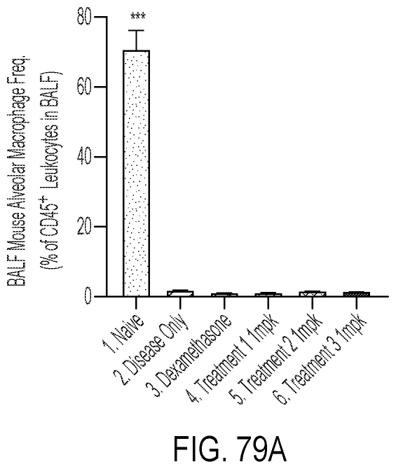

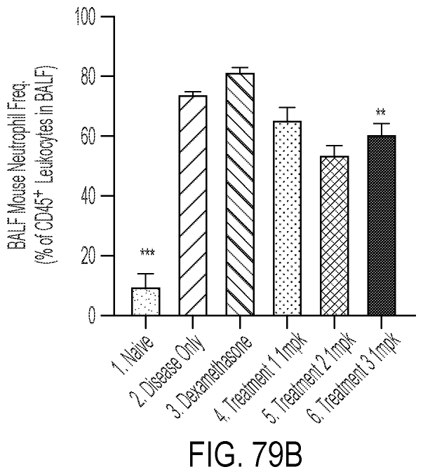

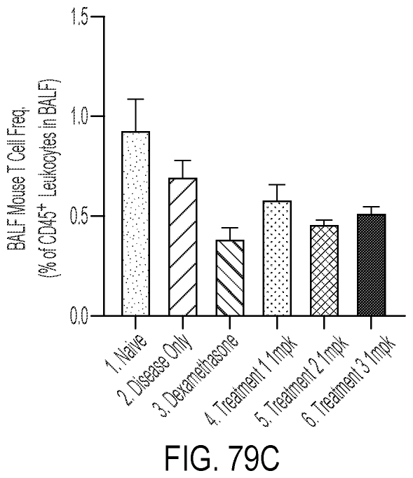

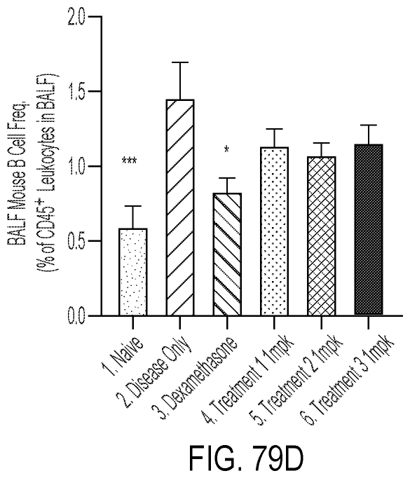

BALF Neutrophils: Cellularity analysis in BALF showed that LPS treatment reduced mouse alveolar macrophage (AM) (FIG. 79A) and enhanced neutrophils (Neu) (FIG. 79B) and B cells recruitment (FIG. 79D) in the lung (Tables 44-46). BALF mouse T cells frequency is shown in FIG. 79C. Treatment of Treatment 2 at 1 mpk significantly repressed neutrophils recruitment in BALF when compared to disease only group (Table 45).

TABLE 45
% Change Relative to LPS in BALF

| Group | CD45+ cells | AM | Neu | T-cells | B-cells |
|---|---|---|---|---|---|
| 2. Disease Only | 100% | 100% | 100% | 100% | 100% |
| 4. Treatment-1 1 mpk | 83% | 56% | 79% | 82% | 73% |
| 5. Treatment-2 1 mpk | 57% | 83% | 42% | 41% | 51% |
| 6. Treatment-3 1 mpk | 96% | 126% | 82% | 77% | 90% |

TABLE 46
% Change Relative to GFP in BALF

| Group | CD45+ cells | AM | Neu | T-cells | B-cells |
|---|---|---|---|---|---|
| 2. Disease Only | 121% | 180% | 127% | 122% | 136% |
| 4. Treatment-1 1 mpk | 100% | 100% | 100% | 100% | 100% |
| 5. Treatment-2 1 mpk | 68% | 149% | 53% | 49% | 70% |
| 6. Treatment-3 1 mpk | 116% | 226% | 104% | 94% | 123% |

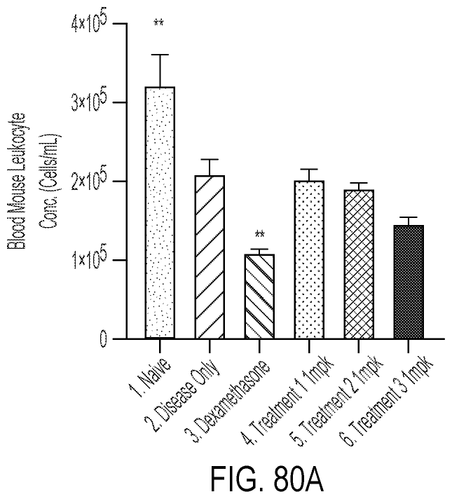

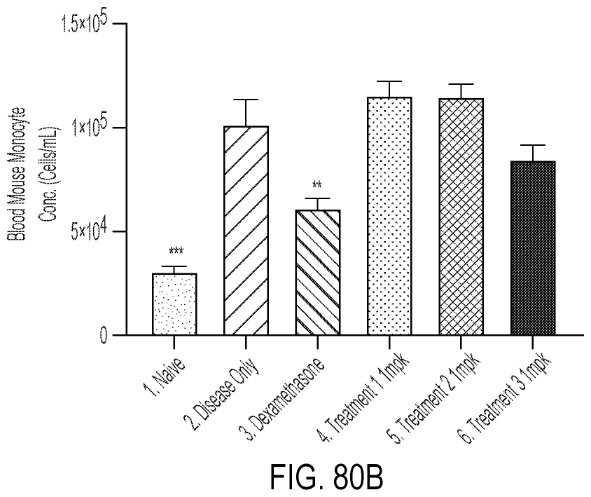

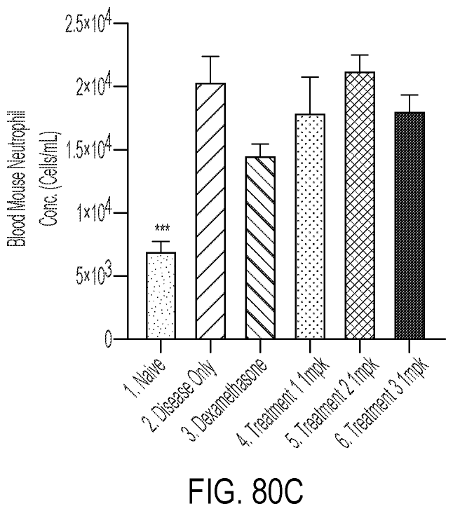

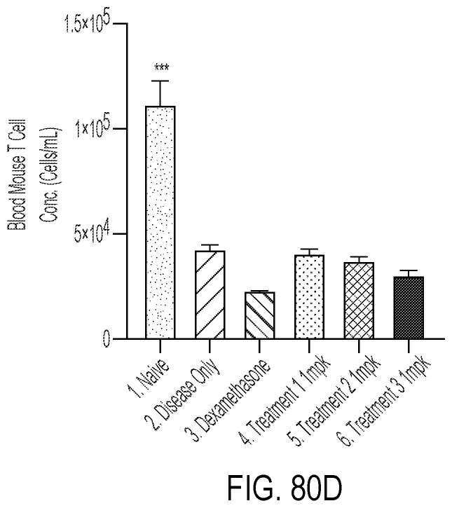

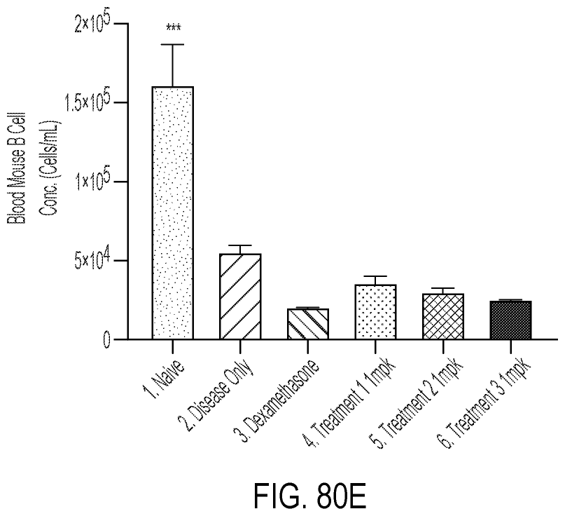

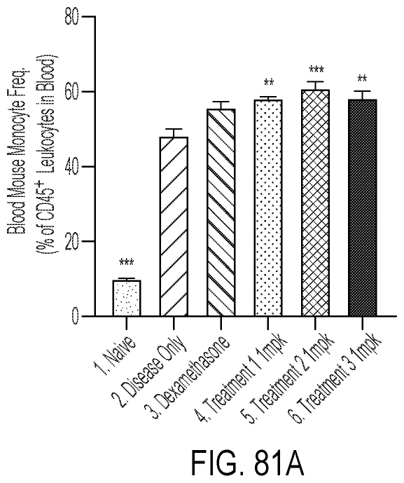

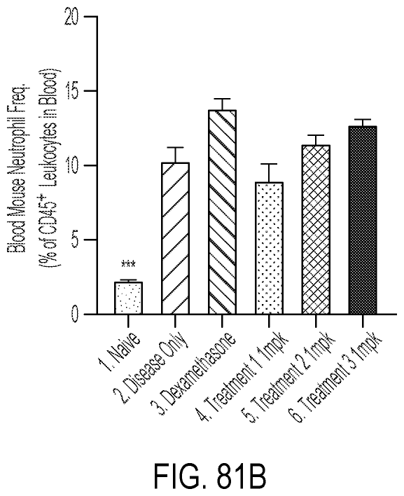

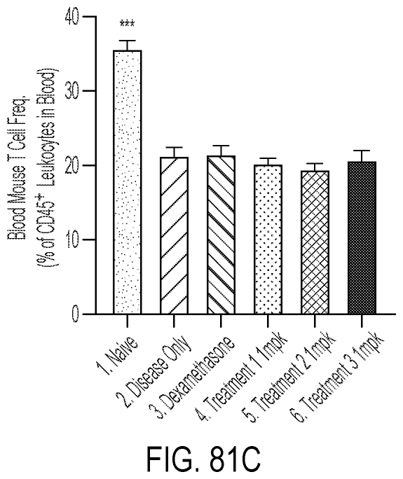

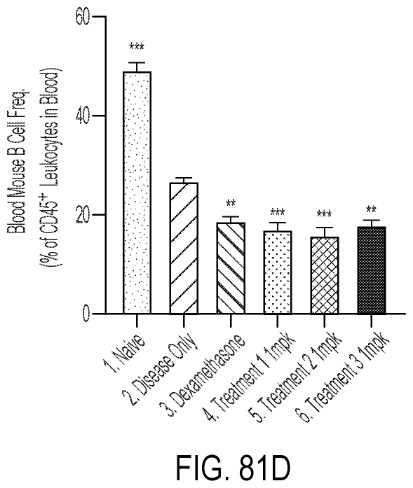

Blood CD45+: Cellularity analysis in blood cells (FIGS. 80A-80E and 81A-81D, Tables 47-49) show that LPS treatment reduced mouse T and B Cells and enhanced monocytes and neutrophils population in the peripheral circulation system. Treatments of all expression repressors significantly enhanced blood monocyte frequency and reduced blood B cells frequency.

TABLE 47
Average cells/mL Blood

| Group | CD45+ cells | AM | Neu | T-cells | B-cells |
|---|---|---|---|---|---|
| 2. Disease Only | 206653.5 | 100559 | 20234.2 | 41871.4 | 54012.9 |
| 4. Treatment-1 | 199986.7 | 114533 | 17797.6 | 39724.4 | 34455.4 |
| 1 mpk | | | | | |
| 5. Treatment-2 | 188581.6 | 113826 | 21126.8 | 36311.5 | 28866.1 |
| 1 mpk | | | | | |
| 6. Treatment-3 | 143446.4 | 83507.8 | 17947.2 | 29402.2 | 24116.7 |
| 1 mpk | | | | | |

TABLE 48
% Change Relative to LPS in Blood

| Group | CD45+ cells | AM | Neu | T-cells | B-cells |
|---|---|---|---|---|---|
| 2. Disease Only | 100% | 100% | 100% | 100% | 100% |
| 4. Treatment-1 1 mpk | 97% | 114% | 88% | 95% | 64% |
| 5. Treatment-2 1 mpk | 91% | 113% | 104% | 87% | 53% |
| 6. Treatment-3 1 mpk | 69% | 83% | 89% | 70% | 45% |

TABLE 49
% Change Relative to GFP in Blood

| Group | CD45+ cells | AM | Neu | T-cells | B-cells |
|---|---|---|---|---|---|
| 2. Disease Only | 103% | 88% | 114% | 105% | 157% |
| 4. Treatment-1 1 mpk | 100% | 100% | 100% | 100% | 100% |
| 5. Treatment-2 1 mpk | 94% | 99% | 119% | 91% | 84% |
| 6. Treatment-3 1 mpk | 72% | 73% | 101% | 74% | 70% |

### Histopathology Assessment

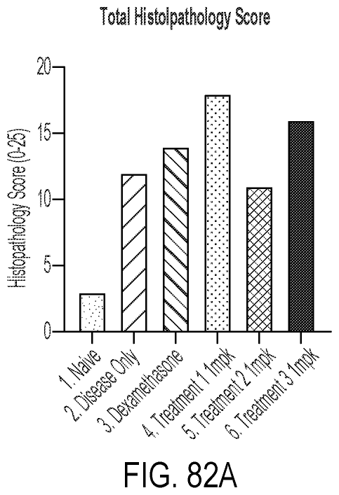

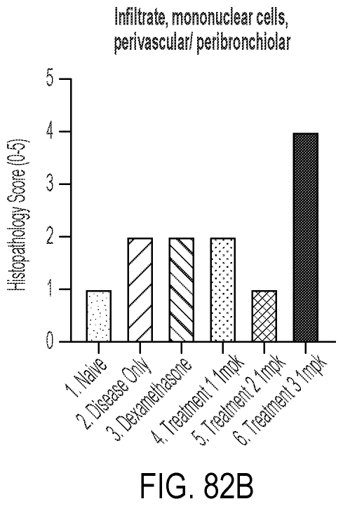

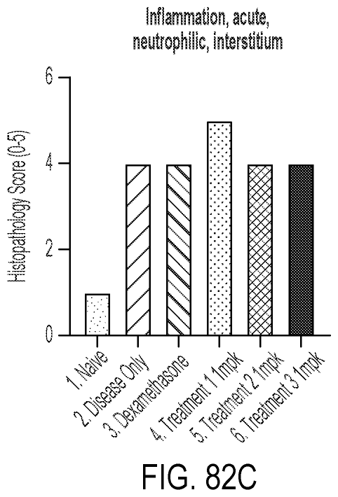

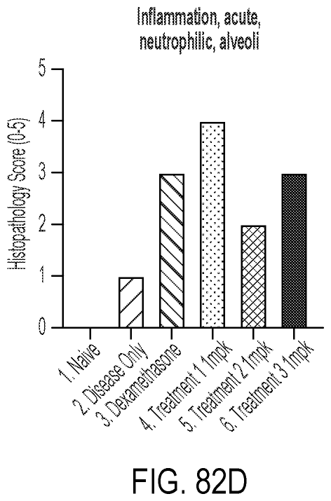

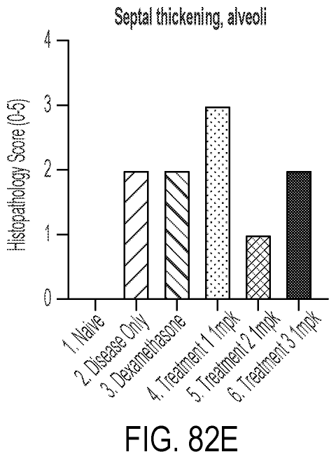

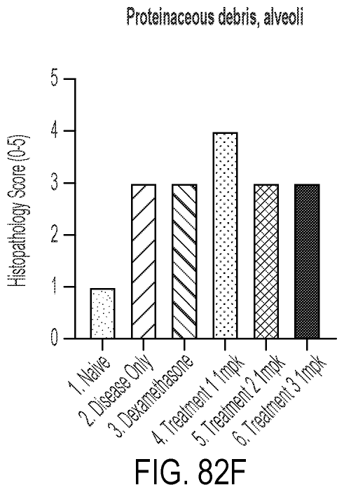

Histology: Among the treatment groups, group 1 had the overall mildest changes. This group represents an untreated control with only minor lesions. Among the treatment groups 2-8, group 5 animal 509 had the mildest disease. This lung has notably milder lesions than all of the other sections except group 1. Group 4 animal 402 and group 6 animal 603 had the most significant lesions of all groups. However, groups 2, 3, 4, 6, 7, and 8 are not notably different from an overall histologic viewpoint. The lesions are approximately similar severity with only modest variations across the scored parameters. These data are shown in FIGS. 82A-82F.

For Group 1, animal 108 was further examined. A single fragment of lung lobe was examined. The lobe was marginally inflated centrally and better inflated peripherally. The section did not include major bronchi. Only smaller bronchioles were present. Bronchioles were devoid of exudate. There was scant visible peribronchial cuffing characterized by isolated infiltrations of a few mononuclear cells. The alveolar wall thickness was altered by atelectasis. The more collapsed areas were more densely cellular. Overall cellularity of the pulmonary interstitium was low and limited to sparse infiltrates of mononuclear cells and a few neutrophils. Alveoli contained some light pale staining material with no visible inflammatory cells. The more peripheral areas of the lung were more well defined with thin alveolar walls. The pleura was devoid of microscopic lesions.

For Group 2, animal 206 was further examined. A single fragment of lung lobe was examined. The lobe was marginally inflated centrally and better inflated peripherally. The section included major bronchi. The bronchi were devoid of exudate. There were some visible peribronchial infiltrations of inflammatory cells. The infiltrates included neutrophils and a few mononuclear cells. The bronchial lining was intact. The alveolar wall thickness was altered by atelectasis. Overall cellularity was low. Most of the visible cells were mononuclear cells. Alveoli contained some pale staining flocculent material and relatively sparse alveolar mononuclear cells. The more peripheral areas showed finer septal details and relatively sparse interstitial cells. Cells in the alveolar lumen were sparse. There was focal hemorrhage in the parenchyma. The pleura was devoid of microscopic lesions.

For Group 3, animal 304 was further examined. A single fragment of lung lobe was examined. The lobe was marginally inflated throughout most of the lung. The section did not include any major bronchi but included smaller bronchioles. Many of the smaller airways were cuffed by light infiltrates of a few neutrophils and mononuclear cells. The bronchial lumens contained no exudate. The alveolar walls were minimally thickened. There was a diffuse light infiltrate of mononuclear cells. Alveoli in many areas contained light accumulations of a few mononuclear cells. Some alveoli contained light accumulations of pale staining flocculent material. Some focal areas of more extensive cellular infiltrates were noted, but these were sparse. The pleura was devoid of significant microscopic lesions.

For Group 4, animal 402 was further examined. A single fragment of lung lobe was examined. The lobe was marginally inflated throughout most of the lung. The section included a few larger bronchi. Many of the larger airways were surrounded by light infiltrates of neutrophils accompanied by some mild hemorrhage. Some of the larger bronchi contained a small amount of visible mucus. The alveolar septa were more cellular, especially in areas of alveolar collapse. The interstitium contained visible infiltrates of mononuclear cells and neutrophils. The microvasculature was also congested. The alveoli contained more visible flocculent material. The more peripheral areas of the lung were more mildly affected. The parenchyma contained some focal hemorrhage. The pleura was devoid of significant microscopic lesions.

For Group 5, animal 509 was further examined. A single fragment of lung lobe was examined. The lung was mildly under-inflated. The section contained no major bronchi. Some, but not all, of the smaller airways were cuffed by a few mixed inflammatory cells. The lumens contain no exudate, mucus or fluid. Alveolar walls were minimally thickened. There were diffuse light infiltrates of sparse mononuclear cells and a few neutrophils. Alveoli contained some light accumulation of pale staining flocculent material. There were sparse mononuclear cells in alveoli. The pleura was devoid of significant microscopic lesions.

For Group 6, animal 603 was further examined. A single fragment of lung lobe was examined. The lung was mildly under-inflated. The section included a few major bronchi. Several of the bronchioles were cuffed by mixed inflammatory cells accompanied by light hemorrhage and some focal areas of edema. The airway lumens contain no fluid, exudate, or mucus. Alveolar walls were minimally thickened and there was a diffuse light infiltrate of mixed mononuclear cells and a few neutrophils. A few focal areas of increased cellularity were noted. Some of these were associated with alveolar collapse (atelectasis). More central alveoli contained light accumulations of pale staining flocculent material. A few cells were dispersed in alveoli. The more peripheral areas were similar but milder changes. The pleura was devoid of significant lesions.

### EQUIVALENTS

It is to be understood that while the invention has been described in conjunction with the detailed description thereof, the foregoing description is intended to illustrate and not limit the scope of the invention, which is defined by the scope of the appended claims. Some aspects, advantages, and modifications are within the scope of the following claims.

What is claimed is:

1. An expression repressor comprising:
a first targeting moiety that binds to a target site, wherein the target site is within an E1 cis-acting regulatory element of a CXCL locus or an E2 cis-acting regulatory element of a CXCL locus, and
optionally, a first effector moiety,
wherein the expression repressor is capable of decreasing expression of a CXCL gene.

2. The expression repressor of claim 1, wherein the target site is within genomic coordinates chr4: 74591400-74593000 or chr4:74982639-74983600 (based on hg19 human genome reference assembly).

3. An expression repressor comprising:
a first targeting moiety that binds to a target site within genomic coordinates chr4: 74591400-74593000 or chr4:74982639-74983600 (based on hg19 human genome reference assembly), and
optionally, a first effector moiety,
wherein the expression repressor is capable of decreasing expression of a CXCL gene.

4. The expression repressor of any of claims 1-3, wherein the target site is chosen from:
a) GRCh37: chr4:74591777-74591797;
b) GRCh37: chr4:74591834-74591854;
c) GRCh37: chr4:74591896-74591916;
d) GRCh37: chr4:74592082-74592102;
e) GRCh37: chr4:74592107-74592127;
f) GRCh37: chr4:74592156-74592176;
g) GRCh37: chr4:74592210-74592230;
h) GRCh37: chr4:74592057-74592077;
i) GRCh37: chr4:74591977-74591997;
j) GRCh37: chr4:74591856-74591876;
k) GRCh37: chr4:74591768-74591790;
l) GRCh37: chr4:74591844-74591866;
m) GRCh37: chr4:74591892-74591914;
n) GRCh37: chr4:74592088-74592110;
o) GRCh37: chr4:74982748-74982770;
p) GRCh37: chr4:74982841-74982863;
q) GRCh37: chr4:74982882-74982904;
r) GRCh37: chr4:74982960-74982982;
s) GRCh37: chr4:74983108-74983130; and
t) GRCh37: chr4:74983181-74983203.

5. The expression repressor of claim 1-4, wherein the first targeting moiety binds within 500, 300, 200, 100, or 50 nucleotides upstream or downstream of a target site chosen from:
a) GRCh37: chr4:74591777-74591797;
b) GRCh37: chr4:74591834-74591854;
c) GRCh37: chr4:74591896-74591916;
d) GRCh37: chr4:74592082-74592102;
e) GRCh37: chr4:74592107-74592127;
f) GRCh37: chr4:74592156-74592176;
g) GRCh37: chr4:74592210-74592230;
h) GRCh37: chr4:74592057-74592077;
i) GRCh37: chr4:74591977-74591997;
j) GRCh37: chr4:74591856-74591876;
k) GRCh37: chr4:74591768-74591790;
l) GRCh37: chr4:74591844-74591866;
m) GRCh37: chr4:74591892-74591914;
n) GRCh37: chr4:74592088-74592110;
o) GRCh37: chr4:74982748-74982770;
p) GRCh37: chr4:74982841-74982863;
q) GRCh37: chr4:74982882-74982904;
r) GRCh37: chr4:74982960-74982982;
s) GRCh37: chr4:74983108-74983130; and
t) GRCh37: chr4:74983181-74983203.

6. An expression repressor comprising:
a first targeting moiety that binds a target site comprising at least 15, 16, 17, 18, 19, 20, 21, 22, 23, 24, 25, 26, 27, 28, or 29, nucleotides of the sequence of any one of SEQ ID NOs: 162 or 163, and
optionally, a first effector moiety,
wherein the expression repressor is capable of decreasing expression of a CXCL gene.

7. An expression repressor comprising:
a first targeting moiety that binds to a target site, wherein the target site is within an IL-8 promoter, and
optionally, a first effector moiety,
wherein the expression repressor is capable of decreasing expression of IL-8.

8. An expression repressor comprising:
a first targeting moiety that binds to a target site within genomic coordinates GRCh37:
chr4:74606162-74606184, or GRCh37: chr4: 74605723-74606223 (based on hg19 human genome reference assembly)
optionally, a first effector moiety,
wherein the expression repressor is capable of decreasing expression of IL-8.

9. An expression repressor comprising:
a first targeting moiety that binds to a target site within genomic coordinates GRCh37: chr4:
74605223-74606223 (based on hg19 human genome reference assembly)
optionally, a first effector moiety,
wherein the expression repressor is capable of decreasing expression of IL-8.

10. The expression repressor of any of claims 1-4, wherein the target sequence comprises a sequence according to SEQ ID NO: 134.

11. The expression repressor of any one of the preceding claims, wherein the first effector moiety comprises an effector described herein, e.g., KRAB, MQ1, DNMT1, DNMT3A1, DNMT3A2, DNMT3B1, DNMT3B2, DNMT3B3, DNMT3B4, DNMT3B5, DNMT3B6, DNMT3L, EZH2, HDAC8, MeCP2, HP1, RBBP4, REST, FOG1, SUZ12, SETDB1, SETDB2, EHMT2 (i.e., G9A), EHMT1 (i.e., GLP), SUV39H1, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, EZH1, SUV39H2, SETD8, SUV420H1, SUV420H2 or DNMT3, or a functional variant or fragment of any thereof.

12. The expression repressor of any one of the preceding claims, wherein the first effector moiety is linked to the targeting moiety via a linker.

13. The expression repressor of any one of the preceding claims, wherein the first effector moiety is C-terminal of the targeting moiety.

14. The expression repressor of any one of the preceding claims, wherein the first effector moiety is N-terminal of the targeting moiety.

15. The expression repressor of any one of the preceding claims, wherein the first effector moiety is encoded by a nucleotide sequence chosen from any of SEQ ID NOs: 10, 14, 16, 18, 66, 68, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

16. The expression repressor of any one of the preceding claims, wherein the first effector moiety comprises an amino acid sequence according to any of SEQ ID NOs: 11, 12, 13, 15, 17, 19, 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

17. The expression repressor of any one of the preceding claims, wherein the first effector moiety is MQ1 or a functional variant or fragment thereof, e.g., wherein the first effector moiety comprises an amino acid sequence of SEQ ID NO: 11 or 12 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the first effector moiety is C-terminal of the first targeting moiety.

18. The expression repressor of any one of the preceding claims, wherein the first effector moiety is KRAB, or a functional variant or fragment thereof, e.g., wherein the first effector moiety comprises an amino acid sequence of SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the first effector moiety is C-terminal of the first targeting moiety.

19. The expression repressor of any of the preceding claims, wherein the effector moiety comprises a DNA methyltransferase, e.g., MQ1 or a fragment or variant thereof.

20. The expression repressor of any of the preceding claims, wherein the effector moiety comprises a transcription repressor, e.g., comprises KRAB or a fragment or variant thereof.

21. The expression repressor of any of the previous claims, wherein the target site has a length of 15-20, 20-25, 25-30, or 30-35 nucleotides.

22. The expression repressor of any of the previous claims, wherein the first targeting moiety comprises a zinc finger domain or a TAL domain.

23. The expression repressor of claim 22, wherein the zinc finger domain comprises 1, 2, 3, 4, 5, 6, 7, 8, 9, or 10 zinc fingers (and optionally no more than 11, 10, 9, 8, 7, 6, or 5 zinc fingers).

24. The expression repressor of claim 22 or 23, wherein the zinc finger domain comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 zinc fingers.

25. The expression repressor of any one of claims 22-24, wherein the zinc finger domain comprises 3, 7, or 9 zinc fingers.

26. The expression repressor of any of claims 1-21, wherein the first targeting moiety comprises a CRISPR-Cas domain.

27. The expression repressor of any one of the preceding claims, which is capable of decreasing expression of a plurality of CXCL genes (e.g., 2, 3, 4, 5, 6, 7, or 8 CXCL genes).

28. The expression repressor of claim 27, which is capable of decreasing expression of one or more of (e.g., 2, 3, 4, 5, 6, 7, or 8 of) CXCL1, CXCL2, CXCL3, CXCL4, CXCL5, CXCL6, CXCL7, or IL-8.

29. The expression repressor of any of claims 1-28, wherein the first effector moiety is a durable effector moiety or a transient effector moiety.

30. The expression repressor of any of the preceding claims, wherein the first targeting moiety comprises a zinc finger domain, and the first effector moiety comprises a transcription repressor, e.g., KRAB or a fragment or variant thereof.

31. The expression repressor of any of the preceding claims, wherein the first targeting moiety comprises a zinc finger domain, and the first effector moiety comprises an epigenetic modifying moiety, e.g., a DNA methyltransferase, e.g., MQ1 or a fragment or variant thereof.

32. The expression repressor of any of the preceding claims, wherein the first targeting moiety comprises an amino acid sequence according to SEQ ID NO: 114, or a sequence having at least 80, 85, 90, 95 or 99% identity thereto.

33. The expression repressor of any one of the preceding claims, which comprises an amino acid sequence of SEQ ID NO: 306, or a sequence having at least 80, 85, 90, 95, or 99% identity thereto.

34. The expression repressor of any one of the preceding claims, which comprises an amino acid sequence of any one of SEQ ID NOs: 152-161 or 164-169, or a sequence having at least 80%, 85%, 90%, 95%, 96%, 97%, 98%, 99% identity thereto, or a sequence with no more than 20, 19, 18, 17, 16, 15, 14, 13, 12, 11, 10, 9, 8, 7, 6, 5, 4, 3, 2, or 1 positions of difference thereto.

35. The expression repressor of any of the preceding claims, which: (i) comprises one or more nuclear localization signal sequences (NLS), or (ii) does not comprise an NLS.

36. The expression repressor of any of the preceding embodiments, comprising a first NLS at the N terminus, e.g., wherein the first NLS has a sequence of SEQ ID NO: 63 or 64.

37. The expression repressor of any of the preceding embodiments, comprising an NLS, e.g., a second NLS, at the C terminus, e.g., having a sequence of SEQ ID NO: 63 or 64.

38. The expression repressor of any of the preceding embodiments, wherein the first and the second NLS have the same sequence.

39. The expression repressor of any of embodiments 36-38, wherein the first and the second NLS have different sequences.

40. The expression repressor of any of the preceding embodiments, wherein binding of the expression repressor to the target site increases methylation at a site in the CXCL locus, e.g., increases methylation at the E1 cis-acting regulatory element of the CXCL locus or the E2 cis-acting regulatory element of the CXCL locus.

41. A system comprising:
a) a first expression repressor according to any of claims 1-40, and
b) a second expression repressor, e.g., a second expression repressor that decreases expression of a CXCL gene.

42. The system of claim 41, wherein the second expression repressor comprises:
a second targeting moiety that binds to a second target site within the CXCL locus, and
optionally, a second effector moiety.

43. The system of claim 42, wherein second expression repressor binds to the E1 cis-acting regulatory element of the CXCL locus, E2 cis-acting regulatory element of the CXCL locus, or IL8 promoter.

44. The system of claim 42 or 43, wherein the second target site is within coordinates GRCh37: chr4:74606162-74606184, GRCh37: chr4: 74605723-74606223, or GRCh37: chr4: 74605223-74606223, or within 1 kb 5′ or 3′ thereof.

45. The system of any of claims 42-44, wherein the second target site is GRCh37: chr4:74606162-74606184 or chr4:74606039-74606056.

46. The system of any of claims 42-45, wherein the second targeting moiety is a clustered regulatory interspaced short palindromic repeat (CRISPR) Cas domain.

47. The system of any of claims 42-46, wherein the second targeting moiety comprises an amino acid sequence according to SEQ ID NO: 268, or a sequence having at least 80, 85, 90, 95, or 99% identity thereto.

48. The system of any of claims 42-47, wherein the second expression repressor comprises an amino acid sequence according to SEQ ID NO: 307, or a sequence having at least 80, 85, 90, 95, or 99% identity thereto.

49. The system of any of claims 42-48, wherein:
the target site comprises a sequence according to SEQ ID NO: 134;
the first effector moiety comprises a KRAB sequence;
the second target site comprises a sequence according to SEQ ID NO: 292; and
the second effector moiety comprises a KRAB sequence.

50. A nucleic acid encoding an expression repressor of any of claims 1-40.

51. A nucleic acid encoding: a first expression repressor of any of claims 1-40 and a second expression repressor, e.g., a second expression repressor that decreases expression of a CXCL gene, e.g., an expression repressor of the system of any of claims 41-49.

52. A nucleic acid system comprising:
a) a first nucleic acid encoding a first expression repressor according to any of claims 1-40, and
b) a second nucleic acid encoding a second expression repressor, e.g., a second expression repressor that decreases expression of a CXCL gene, e.g., an expression repressor of the system of any of claims 41-49.

53. The nucleic acid or nucleic acid system of any of claims 50-52, which comprises a region encoding the first targeting moiety, wherein the region encoding the first targeting moiety comprises a nucleotide sequence of any one of SEQ ID NO: 122-131 or 194-199, or a sequence having at least 80%, 85%, 90%, 95%, 96%, 97%, 98%, or 99% identity thereto.

54. The nucleic acid or nucleic acid system of any of claims 50-53, which comprises a region encoding the first effector moiety, wherein the region encoding the first effector moiety comprises a nucleotide sequence of any one of SEQ ID NO: 10, 14, 16, 18, 66, 68, or a sequence having at least 80%, 85%, 90%, 95%, 96%, 97%, 98%, or 99% identity thereto.

55. The nucleic acid or nucleic acid system of any one of claims 50-54, which further comprises a region encoding an NLS.

56. The nucleic acid or nucleic acid system of claim 55, wherein the region encoding the NLS comprises a nucleotide sequence of SEQ ID NO: 63 or 64, or a sequence having at least 80%, 85%, 90%, 95%, 96%, 97%, 98%, or 99% identity thereto.

57. The nucleic acid system of any of claims 52-56, wherein the first nucleic acid and the second nucleic acid are separate molecules.

58. The nucleic acid system of any of claims 52-56, wherein the first nucleic acid and the second nucleic acid are covalently linked.

59. The nucleic acid system of any of claims 52-58, wherein the first nucleic acid comprises a nucleotide sequence according to SEQ ID NO: 302, or a sequence having at least 80%, 85%, 90%, 95%, 96%, 97%, 98%, or 99% identity thereto, that encodes the first targeting moiety, and a nucleotide sequence according to SEQ ID NO: 303, or a sequence having at least 80%, 85%, 90%, 95%, 96%, 97%, 98%, or 99% identity thereto, that encodes the first effector domain.

60. The nucleic acid system of any of claims 52-59, wherein the second nucleic acid comprises a nucleotide sequence according to SEQ ID NO: 304, or a sequence having at least 80%, 85%, 90%, 95%, 96%, 97%, 98%, or 99% identity thereto, that encodes the second targeting moiety, and a nucleotide sequence according to SEQ ID NO: 305, or a sequence having at least 80%, 85%, 90%, 95%, 96%, 97%, 98%, or 99% identity thereto, that encodes the first effector domain.

61. The nucleic acid system of any of claims 52-60, which has a nucleotide sequence according to SEQ ID NO: 301, or a sequence having at least 80%, 85%, 90%, 95%, 96%, 97%, 98%, or 99% identity thereto.

62. The nucleic acid or nucleic acid system of any of claims 50-61, which comprises DNA or RNA (e.g., mRNA).

63. A vector comprising the nucleic acid or nucleic acid system of any one of claims 50-62.

64. A pharmaceutical composition comprising the expression repressor, nucleic acid, or nucleic acid system of any of the preceding claims.

65. The pharmaceutical composition of claim 64, which comprises an LNP, e.g., wherein the nucleic acid or nucleic acid system is formulated as an LNP.

66. A human cell comprising: an expression repressor of any of claims 1-40, a nucleic acid or nucleic acid system of any of claims 50-62, or a vector of claim 63.

67. A human cell having decreased expression of a CXCL gene, wherein the cell was produced by a method comprising contacting the cell with an expression repressor of any of claims 1-40, a nucleic acid or nucleic acid system of any of claims 50-62, or a vector of claim 63.

68. The human cell of claim 67, wherein the human cell has decreased expression of a first and a second CXCL gene.

69. The human cell of claim 67 or 68, wherein the human cell has decreased expression of a third CXCL gene.

70. The human cell of any one of claims 67-69, wherein the human cell has decreased expression of a fourth CXCL gene.

71. The human cell of any one of claims 67-70, wherein the human cell has decreased expression of a fifth CXCL gene.

72. The human cell of any one of claims 67-71, wherein the human cell has decreased expression of a sixth CXCL gene.

73. The human cell of any one of claims 67-72, wherein the human cell has decreased expression of a seventh CXCL gene.

74. The human cell of any one of claims 67-73, wherein the human cell has decreased expression of an eighth CXCL gene.

75. The human cell of any one of claims 67-74, wherein the human cell has decreased expression of one or more of (e.g., 2, 3, 4, 5, 6, 7, or 8 of) CXCL1, CXCL2, CXCL3, CXCL4, CXCL5, CXCL6, CXCL7, or IL-8.

76. The human cell of any one of claims 67-75, wherein the human cell has decreased expression of one or more of CXCL1, CXCL2, CXCL3, and IL8.

77. A method of decreasing expression of one or more CXCL genes in a cell, comprising contacting the cell with an expression repressor of any one of claims 1-40, a system of any one of claims 41-49 a nucleic acid of any one of claims 50-62, or a vector of claim 63.

78. A method of decreasing expression of one or more CXCL genes in a cell, comprising contacting the cell with an expression repressor, or a nucleic acid comprising a sequence encoding the expression repressor, wherein the expression repressor comprises:
a first targeting moiety that binds to a target site, wherein the target site is within an E1 cis-acting regulatory element of a CXCL locus or an E2 cis-acting regulatory element of a CXCL locus, and
optionally, a first effector moiety,
thereby decreasing expression of a CXCL gene.

79. The method of claim 77 or 78, wherein the target site is within genomic coordinates chr4: 74591400-74593000 or chr4:74982639-74983600 (based on hg19 human genome reference assembly).

80. The method of any one of claims 77-79, wherein expression of one or more of (e.g., 2, 3, 4, 5, 6, 7, or 8 of) CXCL1, CXCL2, CXCL3, CXCL4, CXCL5, CXCL6, CXCL7, or IL-8 is decreased.

81. The method of any one of claims 77-80, wherein expression is decreased for at least 1, 2, 3, 4, 5, 6, 7, 10, or 14 days, or at least 1, 2, 3, 4, or 5 weeks.

82. The expression repressor, the human cell, the system, or the method of any of the preceding claims, wherein the cell is a cell of a subject having an inflammatory disease, e.g., an immune mediated inflammatory disease.

83. The expression repressor, the human cell, the system, or the method of claim 82, wherein the inflammatory disease is an autoimmune disorder, e.g., rheumatoid arthritis.

84. The expression repressor, the human cell, the system, or the method of claim 82 or 83, wherein the inflammatory disease is associated with a pathogenic infection, e.g., viral infection, e.g., SARS-CoV2 infection.

85. The expression repressor, the human cell, the system, or the method of any of claims 82-84, wherein the inflammatory disease is associated with a superinfection, e.g., infection caused by two or more pathogenic agents, e.g., by a virus and a bacterium, (e.g., by SARS-CoV2 and Streptococcus pneumoni), e.g., by a virus and a fungus, (e.g., by SARS-CoV2 and mucormycosis).

86. The expression repressor, the human cell, the system, or the method of any of the preceding claims, wherein the cell is a cell of a subject having rheumatoid arthritis, inflammatory, arthritis, gout, asthma, neutrophilic asthma, neutrophilic dermatosis, paw edema, acute respiratory disease syndrome (ARDS), COVID-19, psoriasis, inflammatory bowel disease, infection (e.g., by a pathogen, e.g., a bacteria, a viruses, or a fungus), external injury (e.g., scrapes or foreign objects), effects of radiation or chemical injury, osteoarthritis, osteoarthritic joint pain, joint pain, inflammatory pain, acute pain, chronic pain, cystitis, bronchitis, dermatitis, dermatosis, cardiovascular disease, neurodegenerative disease, liver disease, lung disease, kidney disease, pain, swelling, stiffness, tenderness, redness, warmth, or elevated biomarkers related to disease states (e.g., cytokines, chemokines, growth factors, immune receptors, infection markers, or inflammatory markers).

87. The expression repressor, the human cell, the system, or the method of any of the preceding claims, wherein the cell is a cell of a subject having rheumatoid arthritis, psoriasis, or inflammatory bowel disease.

88. The expression repressor, the human cell, the system, or the method of any of the preceding claims, wherein the cell is a cell of a subject having rheumatoid arthritis, gout, neutrophilic asthma, neutrophilic dermatosis, acute respiratory disease syndrome (ARDS), or COVID-19.

89. The expression repressor, the human cell, the system, or the method of any of claims 1-81, wherein the cell is a cell of a subject having cancer.

90. The expression repressor, the human cell, the system, or the method of claim 89, wherein the cancer is lung cancer (e.g., non-small cell lung cancer), breast cancer, hepatocellular carcinoma (HCC), prostate cancer, colon cancer, skin cancer, cervical cancer, ovarian cancer, uterine endometrioid carcinoma, endometrial cancer, mature B-cell lymphoma, bladder cancer, esophagogastric cancer, esophageal adenocarcinoma, bone cancer, melanoma, hepatobiliary cancer, thyroid cancer, mature B-cell neoplasms, glioma, head-neck squamous cell carcinoma, kidney renal clear cell carcinoma, pancreatic cancer (e.g., pancreatic ductal adenocarcinoma), sarcoma, or stomach adenocarcinoma.

91. The expression repressor, the human cell, the system, or the method of any of the preceding claims, wherein the cell is situated in a subject.

92. The method of any of claims 77-90, wherein the cell is ex vivo.

93. The method of any of claims 77-92, wherein the cell is a mammalian cell, e.g., a human cell.

94. The method of any of claims 77-93, wherein the cell is a somatic cell.

95. The method of any of claims 77-94, wherein the cell is a primary cell.

96. The method of any of claims 77-95, wherein the step of contacting is performed ex vivo.

97. The method of claim 96, further comprising, prior to the step of contacting, a step of removing the cell (e.g., mammalian cell) from a subject.

98. The method of either of claim 96 or 87, wherein further comprising, after the step of contacting, a step of (b) administering the cells (e.g., mammalian cells) to a subject.

99. The method of any of claims 77-95, wherein the step of contacting comprises administering a composition comprising the expression repressor to a subject.

100. The method of claim 99, wherein the expression repressor is administered as a monotherapy.

101. The method of claim 99, wherein the expression repressor is administered in combination with a second therapeutic agent.

102. A reaction mixture comprising a cell (e.g., a human cell, e.g., a primary human cell) and an expression repressor, or system of any of claims 1-49.

103. A method of treating a subject having an inflammatory disorder, comprising:
administering to the subject an expression repressor, system, nucleic acid, nucleic acid system, or reaction mixture of any of claims 1-102 in an amount sufficient to treat the disorder (e.g., inflammatory disorder),
thereby treating the disorder (e.g., inflammatory disorder).

104. The method of claim 103, wherein the inflammatory disorder is rheumatoid arthritis, psoriasis, or inflammatory bowel disease.

105. The method of claim 103 or 104, wherein the inflammatory disorder is rheumatoid arthritis, gout, neutrophilic asthma, neutrophilic dermatosis, acute respiratory disease syndrome (ARDS), alcohol hepatitis, chronic obstructive pulmonary disease (COPD), or COVID-19.

106. The method of any of claims 103-105, wherein the inflammatory disorder is an autoimmune disorder, e.g., rheumatoid arthritis.

107. The method of any of claims 103-106, wherein the inflammatory disease is associated with a pathogenic infection, e.g., viral infection, e.g., SARS-CoV2 infection.

108. The method of any of claims 103-107, wherein the inflammatory disease is associated with a superinfection, e.g., infection caused by two or more pathogenic agents, e.g., by a virus and a bacterium, (e.g., by SARS-CoV2 and Streptococcus pneumoni), e.g., by a virus and a fungus, (e.g., by SARS-CoV2 and mucormycosis).

109. A method of treating a subject having cancer, comprising:
administering to the subject an expression repressor, system, nucleic acid, nucleic acid system, or reaction mixture of any of claims 1-102 in an amount sufficient to treat the cancer,
thereby treating the cancer.

110. The method of claim 109, wherein the cancer is lung cancer (e.g., non-small cell lung cancer), breast cancer, hepatocellular carcinoma (HCC), prostate cancer, colon cancer, skin cancer, cervical cancer, ovarian cancer, uterine endometrioid carcinoma, endometrial cancer, mature B-cell lymphoma, bladder cancer, esophagogastric cancer, esophageal adenocarcinoma, bone cancer, melanoma, hepatobiliary cancer, thyroid cancer, mature B-cell neoplasms, glioma, head-neck squamous cell carcinoma, kidney renal clear cell carcinoma, pancreatic cancer (e.g., pancreatic ductal adenocarcinoma), sarcoma, or stomach adenocarcinoma.

111. The method of any of claims 77-101 or 103-110, wherein the subject has an E1 cis-acting regulatory element sequence comprising the sequence of SEQ ID NO: 162, or a sequence with no more than 8, 7, 6, 5, 4, 3, 2, or 1 alterations relative thereto.

112. The method of any of claims 77-101 or 103-110, wherein the subject has an E2 cis-acting regulatory element sequence comprising the sequence of SEQ ID NO: 163, or a sequence with no more than 8, 7, 6, 5, 4, 3, 2, or 1 alterations relative thereto.


SEQUENCE LISTING (386 sequences)

SEQ ID NO: 1 - DNA, 20 bp, synthetic construct
  source 1..20: mol_type = other DNA
  misc_difference 1: note = a, c, g, t, unknown or other
  misc_difference 3: note = a, c, g, t, unknown or other
nbndccdshagrkgghrshv

SEQ ID NO: 2 - DNA, 20 bp, synthetic construct
  source 1..20: mol_type = other DNA
  misc_difference 18: note = a, c, g, t, unknown or other
  misc_difference 20: note = a, c, g, t, unknown or other
vhsrhggkrgahsdccdnbn

SEQ ID NO: 3-4 - skipped (2 entries)

SEQ ID NO: 5 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 1053 aa, synthetic construct
AKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 8 - DNA, 4101 bp, synthetic construct
  source 1..4101: mol_type = other DNA
gacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgac

SEQ ID NO: 9 - DNA, 3159 bp, synthetic construct
  source 1..3159: mol_type = other DNA
gccaagcggaactacatcctgggcctggccatcggcatcaccagcgtgggctacggcatcatcgactacgagacccgggacgtgatcgacgccggcgtgcggctgttcaaggaggccaacgtggagaacaacgagggccggcggagcaagcggggcgcccggcggctgaagcggcggcggcggcaccggatccagcgggtgaagaagctgctgttcgactacaacctgctgaccgaccacagcgagctgagcggcatcaacccctacgaggcccgggtgaagggcctgagccagaagctgagcgaggaggagttcagcgccgccctgctgcacctggccaagcggcggggcgtgcacaacgtgaacgaggtggaggaggacaccggcaacgagctgagcaccaaggagcagatcagccggaacagcaaggccctggaggagaagtacgtggccgagctgcagctggagcggctgaagaaggacggcgaggtgcggggcagcatcaaccggttcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcacccgggacgagaacgagaagctggagtactacgagaagttccagatcatcgagaacgtgttcaagcagaagaagaagcccaccctgaagcagatcgccaaggagatcctggtgaacgaggaggacatcaagggctaccgggtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaacagcgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgagccagcagaaggagatccccaccaccctggtggacgacttcatcctgagccccgtggtgaagcggagcttcatccagagcatcaaggtgatcaacgccatcatcaagaagtacggcctgcccaacgacatcatcatcgagctggcccgggagaagaacagcaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggacgccatcatcccccggagcgtgagcttcgacaacagcttcaacaacaaggtgctggtgaagcaggaggagaacagcaagaagggcaaccggacccccttccagtacctgagcagcagcgacagcaagatcagctacgagaccttcaagaagcacatcctgaacctggccaagggcaagggccggatcagcaagaccaagaaggagtacctgctggaggagcgggacatcaaccggttcagcgtgcagaaggacttcatcaaccggaacctggtggacacccggtacgccacccggggcctgatgaacctgctgcggagctacttccgggtgaacaacctggacgtgaaggtgaaatccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaaccggaagctgatcaacgacaccctgtacagcacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagccggaacaaggtggtgaagctgagcctgaagccctaccggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtaccgggtgatcggcgtgaacaacgacctgctgaaccggatcgaggtgaacatgatcgacatcacctaccgggagtacctggagaacatgaacgacaagcggcccccccacatcatcaagaccatcgccagcaagacccagagcatcaagaagtacagcaccgacatcctgggcaacctgtacgaggtgaaatccaagaagcacccccagatcatcaagaagggc

SEQ ID NO: 10 - DNA, 1155 bp, Spiroplasma monobiae
  source 1..1155: mol_type = genomic DNA
agcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggc

SEQ ID NO: 11 - protein, 386 aa, synthetic construct
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 12 - protein, 385 aa, synthetic construct
SKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 13 - protein, 96 aa, Homo sapiens
DAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 14 - DNA, 288 bp, synthetic construct
  source 1..288: mol_type = other DNA
gacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtg

SEQ ID NO: 15 - protein, 542 aa, Homo sapiens
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 16 - DNA, 1626 bp, Homo sapiens
  source 1..1626: mol_type = genomic DNA
aaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctg

SEQ ID NO: 17 - protein, 745 aa, Homo sapiens
GQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 18 - DNA, 2235 bp, synthetic construct
  source 1..2235: mol_type = other DNA
ggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatcccc

SEQ ID NO: 19 - protein, 376 aa, Homo sapiens
EEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVV

SEQ ID NO: 20 - DNA, 20 bp, synthetic construct
  source 1..20: mol_type = other DNA
ggggccactagggacaggat

SEQ ID NO: 21 - DNA, 20 bp, synthetic construct
  source 1..20: mol_type = other DNA
agccccaccttgtggtcaga

SEQ ID NO: 22 - DNA, 20 bp, synthetic construct
  source 1..20: mol_type = other DNA
agtgctgccttctgaccaca

SEQ ID NO: 23 - DNA, 20 bp, synthetic construct
  source 1..20: mol_type = other DNA
gctgccttctgaccacaagg

SEQ ID NO: 24 - DNA, 20 bp, synthetic construct
  source 1..20: mol_type = other DNA
ccagtataagccccaccttg

SEQ ID NO: 25 - DNA, 20 bp, synthetic construct
  source 1..20: mol_type = other DNA
ctgcctgtcccataaggagg

SEQ ID NO: 26 - DNA, 20 bp, synthetic construct
  source 1..20: mol_type = other DNA
gcactgcctgtcccataagg

SEQ ID NO: 27 - DNA, 20 bp, synthetic construct
  source 1..20: mol_type = other DNA
ggtcctcctccttatgggac

SEQ ID NO: 28 - DNA, 20 bp, synthetic construct
  source 1..20: mol_type = other DNA
gccttgttttcggctctaga

SEQ ID NO: 29 - DNA, 20 bp, synthetic construct
  source 1..20: mol_type = other DNA
gccatctagagccgaaaaca

SEQ ID NO: 30 - DNA, 20 bp, synthetic construct
  source 1..20: mol_type = other DNA
ccaatgaagatgaaactggg

SEQ ID NO: 31 - DNA, 20 bp, synthetic construct
  source 1..20: mol_type = other DNA
aacgtgcttgcctaagattc

SEQ ID NO: 32 - DNA, 20 bp, synthetic construct
  source 1..20: mol_type = other DNA
agcccttaatcatatctagt

SEQ ID NO: 33 - DNA, 20 bp, synthetic construct
  source 1..20: mol_type = other DNA
cagagcttaagacctgtact

SEQ ID NO: 34 - DNA, 20 bp, synthetic construct
  source 1..20: mol_type = other DNA
gcccaccttgaccttcacaa

SEQ ID NO: 35 - DNA, 20 bp, synthetic construct
  source 1..20: mol_type = other DNA
gttactgcgtaattaccagg

SEQ ID NO: 36 - DNA, 20 bp, synthetic construct
  source 1..20: mol_type = other DNA
tattacatcctacctataag

SEQ ID NO: 37 - DNA, 20 bp, synthetic construct
  source 1..20: mol_type = other DNA
tgggctctggacttagatcg

SEQ ID NO: 38 - DNA, 20 bp, synthetic construct
  source 1..20: mol_type = other DNA
taagtgggctatgtatacac

SEQ ID NO: 39 - DNA, 20 bp, synthetic construct
  source 1..20: mol_type = other DNA
tttctaagtctgtcacaagg

SEQ ID NO: 40 - DNA, 20 bp, synthetic construct
  source 1..20: mol_type = other DNA
aaagtaatatgatctaggaa

SEQ ID NO: 41 - DNA, 20 bp, synthetic construct
  source 1..20: mol_type = other DNA
gttcgagcggctgtgcgagg

SEQ ID NO: 42 - DNA, 20 bp, synthetic construct
  source 1..20: mol_type = other DNA
gctctgtggctctccgagaa

SEQ ID NO: 43 - DNA, 20 bp, synthetic construct
  source 1..20: mol_type = other DNA
gtgtgtgtgtttcaacgtag

SEQ ID NO: 44 - DNA, 20 bp, synthetic construct
  source 1..20: mol_type = other DNA
ggaagtcactgggagctgcg

SEQ ID NO: 45 - DNA, 20 bp, synthetic construct
  source 1..20: mol_type = other DNA
ggccacgggtgtgttcccag

SEQ ID NO: 46 - DNA, 20 bp, synthetic construct
  source 1..20: mol_type = other DNA
atggccatttgcaaaagtca

SEQ ID NO: 47 - DNA, 20 bp, synthetic construct
  source 1..20: mol_type = other DNA
ccaaactagacagataaagc

SEQ ID NO: 48 - DNA, 20 bp, synthetic construct
  source 1..20: mol_type = other DNA
ccagcatgactctagcatgc

SEQ ID NO: 49 - DNA, 20 bp, synthetic construct
  source 1..20: mol_type = other DNA
tggccaaggtctgatatgca

SEQ ID NO: 50 - DNA, 20 bp, synthetic construct
  source 1..20: mol_type = other DNA
tcatgagtcccagaacatgt

SEQ ID NO: 51 - DNA, 20 bp, synthetic construct
  source 1..20: mol_type = other DNA
gcgaaagaagtagtagctaa

SEQ ID NO: 52 - DNA, 20 bp, synthetic construct
  source 1..20: mol_type = other DNA
gactaagactggcaaatctg

SEQ ID NO: 53 - DNA, 20 bp, synthetic construct
  source 1..20: mol_type = other DNA
gactaagaggagccgacatg

SEQ ID NO: 54 - DNA, 20 bp, synthetic construct
  source 1..20: mol_type = other DNA
gaaaaacgggtgttgtgacg

SEQ ID NO: 55 - DNA, 20 bp, synthetic construct
  source 1..20: mol_type = other DNA
tttgtgaactaaggattctg

SEQ ID NO: 56 - DNA, 20 bp, synthetic construct
  source 1..20: mol_type = other DNA
gtccgtgtagagttaccatg

SEQ ID NO: 57 - DNA, 20 bp, synthetic construct
  source 1..20: mol_type = other DNA
gatgtattcacaagaggact

SEQ ID NO: 58 - DNA, 20 bp, synthetic construct
  source 1..20: mol_type = other DNA
aattactacctcatagctag

SEQ ID NO: 59 - DNA, 20 bp, synthetic construct
  source 1..20: mol_type = other DNA
gaaggtagaaatccgccact

SEQ ID NO: 60 - DNA, 20 bp, synthetic construct
  source 1..20: mol_type = other DNA
gaaacgccgaggtaactcat

SEQ ID NO: 61 - DNA, 20 bp, synthetic construct
  source 1..20: mol_type = other DNA
caactaaaatttctagccct

SEQ ID NO: 62 - DNA, 21 bp, synthetic construct
  source 1..21: mol_type = other DNA
gactccagtctttctagaaga

SEQ ID NO: 63 - protein, 6 aa, Simian virus 40
PKKKRK

SEQ ID NO: 64 - protein, 15 aa, unidentified
KRPAATKKAGQAKKK

SEQ ID NO: 65 - protein, 9 aa, Homo sapiens
YPYDVPDYA

SEQ ID NO: 66 - DNA, 1128 bp, Homo sapiens
  source 1..1128: mol_type = genomic DNA
gaggagcccgaggagcccgccgatagcggacaatctctggtgcccgtctacatctacagccccgaatatgtgagcatgtgtgattccctcgccaagatccctaagagagccagcatggtgcattctctgatcgaggcctacgctctgcataagcaaatgaggatcgtgaagcccaaggtcgccagcatggaagagatggccacctttcacaccgatgcctacctccaacatctccagaaggtgtcccaagagggcgacgacgaccaccccgactccattgagtacggactgggctatgattgccccgccaccgagggcatctttgactatgccgccgctatcggcggagctaccatcacagccgcccagtgtctgattgatggcatgtgcaaggtcgccatcaactggtccggaggctggcatcatgccaagaaggatgaggcctccggcttctgttatctgaatgacgccgtgctgggcattctgagactgaggaggaaattcgagaggattctgtacgtggatctggatctgcatcacggagatggagtcgaagatgccttcagcttcaccagcaaggtgatgacagtctctctgcacaagttctcccccggcttctttcccggaaccggcgacgtgtccgacgtgggactgggcaagggaaggtactacagcgtgaacgtgcccattcaagacggcatccaagacgagaagtactaccagatctgcgagtccgtgctcaaggaggtctaccaagccttcaatcctaaggctgtcgtgctccaactgggagctgataccattgctggcgatcccatgtgcagcttcaatatgacacccgtcggaatcggcaagtgcctcaagtacatcctccagtggcagctcgccaccctcattctcggaggaggcggatacaatctggctaataccgccagatgctggacctatctgaccggcgtgattctgggcaaaacactgagcagcgaaatccccgaccacgagtttttcaccgcttacggccccgactacgtgctggagatcacccccagctgcagacccgatagaaacgaaccccatagaatccagcaaattctgaactatatcaagggcaacctcaagcacgtcgtg

SEQ ID NO: 67 - protein, 282 aa, Homo sapiens
GNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLD

SEQ ID NO: 68 - DNA, 846 bp, Homo sapiens
  source 1..846: mol_type = genomic DNA
ggaaatagggctatcagaaccgagaagatcatctgtagggacgtggctagaggctacgagaacgtgcccattccttgcgtgaatggcgtggatggcgaaccttgccccgaggactacaaatacatctccgagaactgcgaaaccagcacaatgaacatcgacagaaacatcacccacctccagcactgcacatgtgtggatgactgctcctccagcaactgtctgtgcggccagctctccatcagatgctggtacgacaaggacggcagactgctgcaagagttcaacaagatcgaaccccctctcatcttcgagtgtaaccaagcttgcagctgctggagaaactgcaagaatagagtggtccagagcggcatcaaggtgagactgcaactgtacagaaccgccaagatgggatggggagtgagggctctgcaaaccattccccaaggcaccttcatctgcgaatacgtgggcgaactgatctccgacgccgaagctgacgtgagagaggacgacagctatctcttcgatctggacaataaggacggcgaggtgtactgcatcgacgctagatattacggcaacatctctagattcatcaaccacctctgcgatcccaacatcattcccgtgagggtgttcatgctgcaccaagatctgaggttccctagaatcgccttcttcagctctagagacatcagaaccggcgaggagctgggcttcgattacggcgatagattctgggacatcaagtccaagtacttcacatgccagtgcggcagcgagaagtgtaagcacagcgctgaggccattgctctggagcagtctagactggccagactggat

SEQ ID NO: 69 - DNA, 7987 bp, synthetic construct
  source 1..7987: mol_type = other DNA
aggaaataagagagaaaagaagagtaagaagaaatataagagccaccatggcccccaagaagaagcggaaggtgggcatccacggcgtgcccgccgcctcgggcgggggtggctcaggaagcgccgctatcgccgaagtgctgctgaatgccagatgcgatctgcatgccgtgaactaccacggcgacacccctctgcatatcgccgctagagagagctaccatgactgtgtgctgctgtttctgagcagaggcgccaatcccgagctcagaaacaaagagggcgacaccgcttgggatctgacacccgagagatccgacgtgtggttcgctctgcaactgaatagaaaactgagactgggcgtcggcaatagggccattagaaccgagaagatcatctgtagggacgtggctaggggctacgagaacgtgcccatcccttgtgtgaacggagtggatggagagccttgccccgaggattacaaatacatcagcgagaactgcgaaacctccaccatgaatatcgatagaaacattacacacctccagcactgtacatgcgtggacgattgcagcagcagcaactgtctgtgcggccaactgagcatcagatgctggtacgacaaggatggcagactgctgcaagagttcaacaagatcgaaccccctctgatcttcgagtgtaaccaagcttgcagctgttggaggaactgcaagaatagggtcgtgcagtccggaatcaaggtgagactgcagctgtatagaacagctaagatgggatggggagtcagagctctgcagaccatcccccaaggcacattcatctgtgagtacgtcggcgaactcatcagcgacgctgaggccgatgtgagggaggacgacagctatctcttcgacctcgacaacaaggacggcgaggtgtactgcatcgacgctagatattacggcaacatcagcagattcatcaaccacctctgcgaccccaatatcatccccgtgagagtgttcatgctccatcaagatctgagattccctaggatcgccttcttcagctctagagacattagaaccggcgaggagctgggattcgactacggcgacaggttctgggacatcaagagcaagtacttcacatgccaatgcggcagcgagaaatgcaagcatagcgccgaggccattgctctggagcagtctagactggctaggctggaccctcaccccgagctgctgcccgaactgggatctctgcctcccgtgaataccggaggtggcggatcgggagacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagtcgggcgggggtggctcaggacagaccggcaaaaagtccgaaaagggccccgtgtgctggaggaagagggtcaagagcgagtacatgaggctgagacagctcaagagatttaggagagccgatgaggtgaagtccatgttctccagcaacagacaaaagattctggagaggaccgagatcctcaaccaagagtggaagcagagaagaatccagcccgtgcacattctgacctccgtgagctctctgaggggcacaagagaatgctccgtcaccagcgatctggacttccccacacaagtgatccccctcaagacactgaacgctgtggccagcgtgcccatcatgtatagctggtcccctctgcaacagaacttcatggtggaggacgagacagtgctgcacaatatcccctacatgggagatgaggtgctggaccaagacggcacctttattgaggagctgattaaaaactacgatggcaaggtgcacggcgatagggagtgtggcttcatcaacgacgagatcttcgtcgagctggtgaatgctctgggccagtataatgacgatgatgacgacgatgacggcgacgaccccgaagagagagaggagaagcaaaaggatctggaggaccatagggacgacaaagagtctagacctcctagaaagttcccctccgacaagatcttcgaagccatctcctccatgttccccgacaagggcacagccgaggaactgaaggagaagtataaggaactcacagagcaacagctgcccggagctctgcctcccgagtgcacccctaacatcgacggccccaacgccaagagcgtgcagagggagcaatccctccacagcttccataccctcttctgcagaagatgctttaaatacgattgctttctccatcctttccacgccacacccaacacctacaagaggaagaacaccgaaaccgctctggacaataaaccttgcggaccccagtgctaccagcatctggaaggagccaaggaatttgccgctgctctgacagccgagagaattaaaacccctcccaaaagacccggcggcagaaggaggggcagactgcctaataacagcagcagacccagcacccctaccattaacgtgctggaatccaaggacaccgacagcgatagagaggccggcacagaaaccggcggagagaacaacgacaaggaggaggaggagaagaaagacgagacatcctccagcagcgaggctaatagcagatgccagacccctatcaagatgaaacctaatatcgagccccccgagaatgtggagtggagcggcgctgaggcctccatgtttagagtgctgatcggaacctactacgacaacttctgcgctatcgctagactgattggcaccaagacatgcagacaagtgtacgagttcagagtcaaggagagctccattatcgcccccgcccccgccgaagatgtggacaccccccccagaaagaagaaaaggaagcatagactgtgggccgcccactgtagaaagatccagctcaaaaaggacggcagcagcaaccacgtgtacaactatcagccttgtgaccaccccagacaaccttgtgattccagctgcccttgcgtgatcgcccagaacttctgcgagaagttctgtcagtgcagcagcgagtgccaaaatagatttcccggatgtaggtgcaaagcccagtgcaataccaagcagtgcccttgctatctggccgtgagagagtgcgatcccgatctgtgtctgacatgtggagctgccgaccattgggacagcaagaatgtgagctgcaagaactgcagcatccaaaggggaagcaaaaaacatctgctgctcgccccttccgatgtggccggatggggaatctttatcaaggaccccgtccagaaaaacgagttcatttccgagtattgcggcgagatcatcagccaagacgaagctgatagaagaggcaaagtgtatgacaaatacatgtgctccttcctcttcaacctcaataatgatttcgtggtggacgccacaaggaagggcaacaagattagattcgccaaccacagcgtcaatcctaactgctatgccaaggtcatgatggtcaacggcgaccacagaattggcatcttcgctaagagggccatccagaccggcgaggaactgttcttcgactatagatactcccaagccgacgctctgaagtacgtgggcatcgagagagagatggaaatccccggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 70 - protein, 2581 aa, synthetic construct
MAPKKKRKVGIHGVPAASGGGGSGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNTGGGGSGDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPGGGGSGKRPAATKKAGQAKKKKGSYPYDVPDYA

SEQ ID NO: 71 - DNA, 6040 bp, synthetic construct
  source 1..6040: mol_type = other DNA
aggaaataagagagaaaagaagagtaagaagaaatataagagccaccatggcccccaagaagaagcggaaggtgggcatccacggcgtgcccgccgcctcgggcgggggtggctcaggaagcgccgctatcgccgaagtgctgctgaatgccagatgcgatctgcatgccgtgaactaccacggcgacacccctctgcatatcgccgctagagagagctaccatgactgtgtgctgctgtttctgagcagaggcgccaatcccgagctcagaaacaaagagggcgacaccgcttgggatctgacacccgagagatccgacgtgtggttcgctctgcaactgaatagaaaactgagactgggcgtcggcaatagggccattagaaccgagaagatcatctgtagggacgtggctaggggctacgagaacgtgcccatcccttgtgtgaacggagtggatggagagccttgccccgaggattacaaatacatcagcgagaactgcgaaacctccaccatgaatatcgatagaaacattacacacctccagcactgtacatgcgtggacgattgcagcagcagcaactgtctgtgcggccaactgagcatcagatgctggtacgacaaggatggcagactgctgcaagagttcaacaagatcgaaccccctctgatcttcgagtgtaaccaagcttgcagctgttggaggaactgcaagaatagggtcgtgcagtccggaatcaaggtgagactgcagctgtatagaacagctaagatgggatggggagtcagagctctgcagaccatcccccaaggcacattcatctgtgagtacgtcggcgaactcatcagcgacgctgaggccgatgtgagggaggacgacagctatctcttcgacctcgacaacaaggacggcgaggtgtactgcatcgacgctagatattacggcaacatcagcagattcatcaaccacctctgcgaccccaatatcatccccgtgagagtgttcatgctccatcaagatctgagattccctaggatcgccttcttcagctctagagacattagaaccggcgaggagctgggattcgactacggcgacaggttctgggacatcaagagcaagtacttcacatgccaatgcggcagcgagaaatgcaagcatagcgccgaggccattgctctggagcagtctagactggctaggctggaccctcaccccgagctgctgcccgaactgggatctctgcctcccgtgaataccggaggtggcggatcgggagacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagtcgggcgggggtggctcagacgctaagtctctgaccgcttggagcagaacactggtcaccttcaaggacgtgttcgtcgacttcacaagagaggagtggaaactgctggacaccgcccagcagatcctctatagaaacgtcatgctggagaactacaagaatctggtgtctctgggctaccagctgaccaagcccgacgtgattctgaggctggagaagggcgaggagccttggctggtggagagagagatccaccaagaaacccaccccgacagcgaaaccgccttcgagatcaagagcagcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 72 - protein, 1932 aa, synthetic construct
MAPKKKRKVGIHGVPAASGGGGSGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNTGGGGSGDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVGGGGSGKRPAATKKAGQAKKKKGSYPYDVPDYA

SEQ ID NO: 73 - protein, 1497 aa, synthetic construct
MAPKKKRKVGIHGVPAAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGKRPAATKKAGQAKKKKARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 74 - protein, 1811 aa, synthetic construct
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 75 - protein, 1519 aa, synthetic construct
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 76 - protein, 2168 aa, synthetic construct
MAPKKKRKVGGSGGSGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKGS

SEQ ID NO: 77 - protein, 1977 aa, synthetic construct
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSAGGGGSGGGGSGGGGSGPKKKRKVAAAGSNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLSGGKRPAATKKAGQAKKKKGS

SEQ ID NO: 78 - protein, 1809 aa, synthetic construct
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVVGGGGSGKRPAATKKAGQAKKKKGS

SEQ ID NO: 79 - protein, 2572 aa, synthetic construct
MAPKKKRKVGGSGGSGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVVGGGGSGKRPAATKKAGQAKKKKGS

SEQ ID NO: 80 - protein, 2572 aa, synthetic construct
MAPKKKRKVGIHGVPAASGGGGSGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNTGGGGSGDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPGGGGSGKRPAATKKAGQAKKKKGS

SEQ ID NO: 81 - protein, 1923 aa, synthetic construct
MAPKKKRKVGIHGVPAASGGGGSGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNTGGGGSGDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVGGGGSGKRPAATKKAGQAKKKKGS

SEQ ID NO: 82 - protein, 2292 aa, synthetic construct
MAPKKKRKVGGSGGSGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVGGGGSGKRPAATKKAGQAKKKKGS

SEQ ID NO: 83 - skipped

SEQ ID NO: 84 - protein, 1405 aa, synthetic construct
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKGS

SEQ ID NO: 85 - DNA, 7147 bp, synthetic construct
  source 1..7147: mol_type = other DNA
aggaaataagagagaaaagaagagtaagaagaaatataagagccaccatggcccccaagaagaagcggaaggtgggcggcagcggcggcagcggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatccccagcaccggcggcagcggcggcagcggcggcagcggcggcagcggcggcagcggccgacccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagtcgggcgggggtggctcagacgctaagtctctgaccgcttggagcagaacactggtcaccttcaaggacgtgttcgtcgacttcacaagagaggagtggaaactgctggacaccgcccagcagatcctctatagaaacgtcatgctggagaactacaagaatctggtgtctctgggctaccagctgaccaagcccgacgtgattctgaggctggagaagggcgaggagccttggctggtggagagagagatccaccaagaaacccaccccgacagcgaaaccgccttcgagatcaagagcagcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 86 - protein, 2301 aa, synthetic construct
MAPKKKRKVGGSGGSGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVGGGGSGKRPAATKKAGQAKKKKGSYPYDVPDYA

SEQ ID NO: 87-89 - skipped (3 entries)

SEQ ID NO: 90 - DNA, 20 bp, synthetic construct
  source 1..20: mol_type = other DNA
ttattcttgaggcatctgtg

SEQ ID NO: 91 - DNA, 20 bp, synthetic construct
  source 1..20: mol_type = other DNA
aaattccacacatggtcaag

SEQ ID NO: 92 - DNA, 20 bp, synthetic construct
  source 1..20: mol_type = other DNA
gtcagccaaagacattgcac

SEQ ID NO: 93 - DNA, 20 bp, synthetic construct
  source 1..20: mol_type = other DNA
ctgtgtaacagggtctgaat

SEQ ID NO: 94 - DNA, 20 bp, synthetic construct
  source 1..20: mol_type = other DNA
tatagaatattgttaccctg

SEQ ID NO: 95 - DNA, 20 bp, synthetic construct
  source 1..20: mol_type = other DNA
tccttgggaaagtccacaag

SEQ ID NO: 96 - DNA, 20 bp, synthetic construct
  source 1..20: mol_type = other DNA
gacaacaaatgacattgtct

SEQ ID NO: 97 - DNA, 20 bp, synthetic construct
  source 1..20: mol_type = other DNA
gccaaaagattctgtgtccg

SEQ ID NO: 98 - DNA, 20 bp, synthetic construct
  source 1..20: mol_type = other DNA
tcagttaagaaaaggtacag

SEQ ID NO: 99 - DNA, 20 bp, synthetic construct
  source 1..20: mol_type = other DNA
ttcagatcttaatctgtaca

SEQ ID NO: 100 - DNA, 20 bp, synthetic construct
  source 1..20: mol_type = other DNA
actgatggcccatcccctca

SEQ ID NO: 101 - DNA, 100 bp, synthetic construct
  source 1..100: mol_type = other DNA
ttattcttgaggcatctgtggttttagagctagaaatagcaagttaaaataaggctagtccgttatcaacttgaaaaagtggcaccgagtcggtgctttt

SEQ ID NO: 102 - DNA, 100 bp, synthetic construct
  source 1..100: mol_type = other DNA
aaattccacacatggtcaaggttttagagctagaaatagcaagttaaaataaggctagtccgttatcaacttgaaaaagtggcaccgagtcggtgctttt

SEQ ID NO: 103 - DNA, 100 bp, synthetic construct
  source 1..100: mol_type = other DNA
gtcagccaaagacattgcacgttttagagctagaaatagcaagttaaaataaggctagtccgttatcaacttgaaaaagtggcaccgagtcggtgctttt

SEQ ID NO: 104 - DNA, 100 bp, synthetic construct
  source 1..100: mol_type = other DNA
ctgtgtaacagggtctgaatgttttagagctagaaatagcaagttaaaataaggctagtccgttatcaacttgaaaaagtggcaccgagtcggtgctttt

SEQ ID NO: 105 - DNA, 100 bp, synthetic construct
  source 1..100: mol_type = other DNA
tatagaatattgttaccctggttttagagctagaaatagcaagttaaaataaggctagtccgttatcaacttgaaaaagtggcaccgagtcggtgctttt

SEQ ID NO: 106 - DNA, 100 bp, synthetic construct
  source 1..100: mol_type = other DNA
tccttgggaaagtccacaaggttttagagctagaaatagcaagttaaaataaggctagtccgttatcaacttgaaaaagtggcaccgagtcggtgctttt

SEQ ID NO: 107 - DNA, 100 bp, synthetic construct
  source 1..100: mol_type = other DNA
gacaacaaatgacattgtctgttttagagctagaaatagcaagttaaaataaggctagtccgttatcaacttgaaaaagtggcaccgagtcggtgctttt

SEQ ID NO: 108 - DNA, 100 bp, synthetic construct
  source 1..100: mol_type = other DNA
gccaaaagattctgtgtccggttttagagctagaaatagcaagttaaaataaggctagtccgttatcaacttgaaaaagtggcaccgagtcggtgctttt

SEQ ID NO: 109 - DNA, 100 bp, synthetic construct
  source 1..100: mol_type = other DNA
tcagttaagaaaaggtacaggttttagagctagaaatagcaagttaaaataaggctagtccgttatcaacttgaaaaagtggcaccgagtcggtgctttt

SEQ ID NO: 110 - DNA, 100 bp, synthetic construct
  source 1..100: mol_type = other DNA
ttcagatcttaatctgtacagttttagagctagaaatagcaagttaaaataaggctagtccgttatcaacttgaaaaagtggcaccgagtcggtgctttt

SEQ ID NO: 111 - DNA, 100 bp, synthetic construct
  source 1..100: mol_type = other DNA
actgatggcccatcccctcagttttagagctagaaatagcaagttaaaataaggctagtccgttatcaacttgaaaaagtggcaccgagtcggtgctttt

SEQ ID NO: 112 - protein, 204 aa, synthetic construct
LEPGEKPYKCPECGKSFSHKNALQNHQRTHTGEKPYKCPECGKSFSQRANLRAHQRTHTGEKPYKCPECGKSFSRSDKLVRHQRTHTGEKPYKCPECGKSFSQAGHLASHQRTHTGEKPYKCPECGKSFSRNDALTEHQRTHTGEKPYKCPECGKSFSTSGNLTEHQRTHTGEKPYKCPECGKSFSRSDHLTNHQRTHTGKKTS

SEQ ID NO: 113 - protein, 204 aa, synthetic construct
LEPGEKPYKCPECGKSFSSKKALTEHQRTHTGEKPYKCPECGKSFSTSHSLTEHQRTHTGEKPYKCPECGKSFSHKNALQNHQRTHTGEKPYKCPECGKSFSQSSNLVRHQRTHTGEKPYKCPECGKSFSRSDHLTTHQRTHTGEKPYKCPECGKSFSTKNSLTEHQRTHTGEKPYKCPECGKSFSTTGNLTVHQRTHTGKKTS

SEQ ID NO: 114 - protein, 204 aa, synthetic construct
LEPGEKPYKCPECGKSFSTSGNLVRHQRTHTGEKPYKCPECGKSFSRADNLTEHQRTHTGEKPYKCPECGKSFSQSGDLRRHQRTHTGEKPYKCPECGKSFSHKNALQNHQRTHTGEKPYKCPECGKSFSDPGNLVRHQRTHTGEKPYKCPECGKSFSQRANLRAHQRTHTGEKPYKCPECGKSFSDCRDLARHQRTHTGKKTS

SEQ ID NO: 115 - protein, 204 aa, synthetic construct
LEPGEKPYKCPECGKSFSRNDALTEHQRTHTGEKPYKCPECGKSFSDKKDLTRHQRTHTGEKPYKCPECGKSFSRADNLTEHQRTHTGEKPYKCPECGKSFSHKNALQNHQRTHTGEKPYKCPECGKSFSTSHSLTEHQRTHTGEKPYKCPECGKSFSTSGNLVRHQRTHTGEKPYKCPECGKSFSREDNLHTHQRTHTGKKTS

SEQ ID NO: 116 - protein, 204 aa, synthetic construct
LEPGEKPYKCPECGKSFSRKDNLKNHQRTHTGEKPYKCPECGKSFSRADNLTEHQRTHTGEKPYKCPECGKSFSDPGALVRHQRTHTGEKPYKCPECGKSFSDKKDLTRHQRTHTGEKPYKCPECGKSFSDPGALVRHQRTHTGEKPYKCPECGKSFSRSDNLVRHQRTHTGEKPYKCPECGKSFSSPADLTRHQRTHTGKKTS

SEQ ID NO: 117 - protein, 204 aa, synthetic construct
LEPGEKPYKCPECGKSFSRSDELVRHQRTHTGEKPYKCPECGKSFSQSGDLRRHQRTHTGEKPYKCPECGKSFSQRANLRAHQRTHTGEKPYKCPECGKSFSTHLDLIRHQRTHTGEKPYKCPECGKSFSRSDHLTTHQRTHTGEKPYKCPECGKSFSTSGNLTEHQRTHTGEKPYKCPECGKSFSQSGDLRRHQRTHTGKKTS

SEQ ID NO: 118 - protein, 204 aa, synthetic construct
LEPGEKPYKCPECGKSFSRNDALTEHQRTHTGEKPYKCPECGKSFSRRDELNVHQRTHTGEKPYKCPECGKSFSRSDELVRHQRTHTGEKPYKCPECGKSFSRADNLTEHQRTHTGEKPYKCPECGKSFSTSHSLTEHQRTHTGEKPYKCPECGKSFSERSHLREHQRTHTGEKPYKCPECGKSFSTHLDLIRHQRTHTGKKTS

SEQ ID NO: 119 - protein, 204 aa, synthetic construct
LEPGEKPYKCPECGKSFSTSGNLTEHQRTHTGEKPYKCPECGKSFSQSSSLVRHQRTHTGEKPYKCPECGKSFSRSDHLTTHQRTHTGEKPYKCPECGKSFSRSDHLTTHQRTHTGEKPYKCPECGKSFSQSSSLVRHQRTHTGEKPYKCPECGKSFSSKKALTEHQRTHTGEKPYKCPECGKSFSTKNSLTEHQRTHTGKKTS

SEQ ID NO: 120 - protein, 204 aa, synthetic construct
LEPGEKPYKCPECGKSFSTHLDLIRHQRTHTGEKPYKCPECGKSFSDPGNLVRHQRTHTGEKPYKCPECGKSFSRKDNLKNHQRTHTGEKPYKCPECGKSFSDKKDLTRHQRTHTGEKPYKCPECGKSFSRSDELVRHQRTHTGEKPYKCPECGKSFSTHLDLIRHQRTHTGEKPYKCPECGKSFSQSSSLVRHQRTHTGKKTS

SEQ ID NO: 121 - protein, 204 aa, synthetic construct
LEPGEKPYKCPECGKSFSTSHSLTEHQRTHTGEKPYKCPECGKSFSQAGHLASHQRTHTGEKPYKCPECGKSFSTKNSLTEHQRTHTGEKPYKCPECGKSFSDCRDLARHQRTHTGEKPYKCPECGKSFSQAGHLASHQRTHTGEKPYKCPECGKSFSQSSSLVRHQRTHTGEKPYKCPECGKSFSRSDHLTTHQRTHTGKKTS

SEQ ID NO: 122 - DNA, 612 bp, synthetic construct
  source 1..612: mol_type = other DNA
ctggagcctggggagaaaccctacaaatgccctgagtgcgggaagagtttcagccacaagaacgcgttgcagaaccaccaaaggacccatactggagagaaaccgtacaagtgccctgaatgtggaaagtcgttctcccaacgcgccaacctgagagctcaccaacgcactcataccggggaaaagccctataagtgcccggaatgcggcaaaagcttctcccggtccgacaagcttgtgcggcaccagcggactcacactggggagaagccctacaagtgcccagaatgcggaaagagcttcagccaggccggtcatctggcctcgcatcagcggacccacactggcgaaaagccttacaagtgtccggagtgcggaaagtccttctcccggaacgacgcactcaccgaacaccagcgcacccacaccggagagaagccgtataagtgtccagaatgtggcaaatccttctccacatctggcaatctgaccgagcaccagagaacccataccggagaaaagccgtacaaatgtcccgaatgcgggaagtcattttcccgctcggatcacctgaccaaccaccagaggactcacacgggaaagaaaacctca

SEQ ID NO: 123 - DNA, 612 bp, synthetic construct
  source 1..612: mol_type = other DNA
cttgagcccggcgaaaagccctacaagtgcccggaatgcggaaaatcgttcagctcaaagaaggccttgactgaacaccaacggacccacacgggagaaaaaccgtataagtgtccagagtgcgggaagtcattctccacttcccactccctgaccgagcaccagcgcactcacaccggagagaagccttacaagtgtcctgagtgcggaaagtctttcagccacaagaacgcgctgcagaaccaccagaggacacataccggcgaaaaaccctacaaatgcccggagtgtggaaagtccttctcgcaatcctccaatctggtccggcaccagagaacccatactggggaaaaaccttacaaatgtccggagtgcggcaaaagcttttcccgctccgaccacctcaccactcaccaaagaacccacactggggagaagccctataagtgccctgagtgtgggaagtccttcagcaccaagaactcgctcaccgaacaccagcggactcataccggagaaaagccgtacaagtgcccagaatgtggaaagagcttctcgaccaccggcaacctgactgtgcatcagcgcacccataccggcaaaaagacctca

SEQ ID NO: 124 - DNA, 612 bp, synthetic construct
  source 1..612: mol_type = other DNA
ctggagcccggagaaaaaccctataagtgtccggagtgcggaaagtcgttctccacttccggcaaccttgtgcggcaccagcgaacacataccggagaaaagccctacaagtgccctgagtgtgggaagtccttctcccgggcggacaacttgaccgagcaccagagaacccatactggggaaaagccgtataagtgccccgaatgtggaaagtcctttagccaaagcggcgatctcaggcggcatcagcgcacccacactggcgaaaagccatacaagtgcccggagtgtggaaaatctttctcgcacaagaacgcactgcagaatcaccagcggacccataccggggagaagccgtacaagtgtcctgaatgcggaaagagcttctccgaccctggcaacctcgtgcgccaccaacgcactcacaccggtgaaaagccttacaagtgcccagagtgcgggaagtcattcagccagcgggccaacctgagagcccaccaaagaacccacaccggagagaaaccctacaaatgcccggaatgcggcaaatcgttctccgactgccgcgatctggctcggcaccagaggactcacacgggcaaaaagacctca

SEQ ID NO: 125 - DNA, 612 bp, synthetic construct
  source 1..612: mol_type = other DNA
ctggaaccgggagagaaaccctataagtgtccggagtgcggaaagtctttctcccgcaacgacgcgcttaccgagcaccagcgcacccacactggagaaaaaccctacaaatgccctgaatgtggcaaatcattctccgataagaaggacttgaccagacaccagcggacccacaccggagaaaagccgtataagtgccccgagtgtgggaagtcgttcagccgggccgataacctgacggaacaccagagaactcataccggggagaagccgtacaagtgtcctgaatgcgggaagtcctttagccacaagaacgccctgcaaaaccaccaacggacccataccggagagaagccttacaagtgcccggaatgcggaaagagcttctcgacctcccattcgctgactgagcatcagaggacacacaccggcgaaaagccatacaagtgccctgagtgcggcaaatccttctccacttccggcaatctcgtgcggcaccagaggactcacaccggggaaaagccctacaagtgcccagaatgtggaaagtccttcagccgcgaggacaacctccacacccatcagcgcactcacactggcaaaaagacctca

SEQ ID NO: 126 - DNA, 612 bp, synthetic construct
  source 1..612: mol_type = other DNA
ctggagcctggcgaaaagccttacaagtgtcccgaatgcggcaaatcgttctcacggaaggacaaccttaagaaccaccagaggacccataccggagaaaagccatacaaatgcccggagtgcgggaagtccttctcccgcgccgacaacctgactgagcaccaacggacccatacaggggagaagccctataagtgcccggaatgtggaaagtcgttctcggaccccggtgcactcgtgcgccatcagaggacgcacaccggggaaaagccctacaaatgtccggaatgcggaaagtccttttccgacaagaaggatttgaccagacaccagcgcactcacaccggagagaagccgtacaagtgcccagagtgcggcaaaagcttctccgatcctggagctctggtccggcatcagcggacccacaccggtgaaaagccgtataagtgtccggagtgtggaaagagcttctcccggtccgacaatctcgtgcggcaccaacgcacccatactggcgaaaaaccctacaagtgccctgagtgcggaaaaagcttcagctctcctgccgacctgactagacaccagagaacccacactggcaaaaagacctca

SEQ ID NO: 127 - DNA, 612 bp, synthetic construct
  source 1..612: mol_type = other DNA
ctggaacccggagagaagccgtataagtgtcctgaatgcggaaagtccttctcccgctcggacgagcttgtgcggcaccagcgcactcacactggcgaaaagccgtacaaatgccccgaatgcgggaagtccttcagccaatcgggagatctgcgcagacatcagcggacccacaccggagaaaagccttataagtgccctgagtgcgggaaatcgttctcccaacgggcgaacctcagggcccaccaacggactcacacgggggaaaagccctacaagtgtccggagtgtggaaagtcattctccacccacctggacctgatcaggcaccagcggacacataccggagagaaaccgtacaagtgcccggaatgtggaaagagcttctcaagatccgaccacttgaccacccatcagcgcacccatactggggagaagccttacaaatgtcccgaatgtggcaaatcgttttccacctccggcaacctgaccgagcatcagaggactcacaccggcgaaaaaccatacaagtgcccagagtgcggcaaaagcttcagccagtccggcgatctccggagacaccagcgaacccacactggaaagaaaacctct

SEQ ID NO: 128 - DNA, 612 bp, synthetic construct
  source 1..612: mol_type = other DNA
ctggagcctggcgaaaaaccgtacaagtgccccgaatgtggaaagtctttctcccgcaacgacgcgttgaccgaacaccagcggacccataccggtgaaaagccttacaagtgccctgagtgcggcaaaagcttctcccgccgcgatgaactgaacgtgcaccaacgcacccacactggagaaaagccatacaaatgcccggagtgtgggaagtccttctcccggtccgatgagcttgtgcggcaccagagaactcacacaggggaaaagccctataagtgcccagagtgcgggaagtcattcagccgggccgacaacctgaccgagcaccaaaggactcacaccggagagaagccgtataagtgtcctgagtgtggaaagagcttcagcacctcccactccctcaccgaacatcagcggactcatactggggagaagccctacaagtgtcccgaatgcggcaaatccttttcggaacggtcgcacctgagagagcatcagaggacgcacaccggcgaaaagccgtacaaatgtccggagtgcggaaagtcgttctccacccacctcgacctgatcagacaccagcgcacccatactggcaaaaagacctca

SEQ ID NO: 129 - DNA, 612 bp, synthetic construct
  source 1..612: mol_type = other DNA
ctggagcccggagagaaaccgtacaagtgtccagagtgcggcaaatccttctcgacctccggcaacctgactgaacaccagcgcactcacactggagaaaagccgtacaaatgtcctgaatgcggaaagagcttcagccagtcctcctcacttgtgcgccaccagaggacccacaccggagaaaaaccgtataagtgtcctgagtgtgggaagtcattctcaagaagcgatcacctcaccacccatcagcggacccatacgggggagaagccctacaaatgcccggagtgcggaaagtcgttcagccggtccgaccacctgaccacccaccaaagaactcacaccggcgaaaagccttacaagtgccctgaatgtggaaagtccttttcccaatcgtcctccttggtccggcatcagaggactcacacaggagagaagccatacaagtgccccgaatgcgggaagtccttcagctcgaagaaggccctgactgagcaccagcggactcataccggggaaaagccctataagtgcccggaatgcggcaaaagcttctcgaccaagaactccctcaccgagcatcagcgcacccacactggcaaaaagacctct

SEQ ID NO: 130 - DNA, 612 bp, synthetic construct
  source 1..612: mol_type = other DNA
ctggaacccggagagaagccctataagtgtccggagtgcggcaaatccttttccacccatctcgacctgattaggcaccagagaactcacactggggagaagccatacaagtgccccgaatgtggaaagtctttctccgatccgggcaaccttgtccggcaccagaggacccacacgggcgaaaaaccttataagtgccctgagtgtgggaagtccttcagccggaaggacaacctgaagaaccaccaaagaacacacaccggagaaaagccctacaaatgcccagagtgcgggaagtcgttctccgacaagaaggatttgacccggcaccaacggacccacactggcgaaaagccgtacaaatgtcccgaatgcggaaagagcttctcgcgctccgacgagctcgtgcgccatcagcggactcataccggagagaaaccgtacaagtgtcctgagtgcggaaagtccttctcaacccacctggacctgatcagacaccagcgcactcacaccggggaaaagccttacaagtgcccggaatgcggcaaaagcttctcccaatcgagctccctcgtgcggcatcagcgcacccataccggaaagaaaacctca

SEQ ID NO: 131 - DNA, 612 bp, synthetic construct
  source 1..612: mol_type = other DNA
ttggaaccgggggaaaagccgtacaagtgccccgaatgtggcaaatcattctctacctcccactcactgaccgagcaccagaggacccacaccggagagaaaccctacaagtgtccagaatgcggaaagtccttctcgcaagccggtcaccttgcgtcccaccaacgcactcatactggagaaaaaccatacaagtgcccggaatgcggcaaaagcttcagcaccaagaacagcctgactgagcatcagaggactcacaccggcgaaaagccctataagtgtcctgagtgcggcaaatccttctccgattgtcgggacctggccagacaccagcgcactcacacaggagaaaagccttataagtgccctgagtgtggaaagtcgtttagccaagctggtcacctggcatcccatcagcggactcataccggggagaagccttacaaatgtccggagtgcgggaagtcgttcagccagtcctcctccctcgtgcggcaccagagaacccacactggagagaagccctacaaatgccctgaatgtggaaagagcttctcccgctcggaccacctcaccacgcaccagcggacccataccggcaaaaagacctca

SEQ ID NO: 132 - DNA, 21 bp, synthetic construct
  source 1..21: mol_type = other DNA
aggcatctgtgagggaaaatt

SEQ ID NO: 133 - DNA, 21 bp, synthetic construct
  source 1..21: mol_type = other DNA
aatccttgggaaattccacac

SEQ ID NO: 134 - DNA, 21 bp, synthetic construct
  source 1..21: mol_type = other DNA
gccaaagacattgcacaggat

SEQ ID NO: 135 - DNA, 21 bp, synthetic construct
  source 1..21: mol_type = other DNA
taggatccaattcagaccctg

SEQ ID NO: 136 - DNA, 21 bp, synthetic construct
  source 1..21: mol_type = other DNA
acagaggtcaccgtccagaag

SEQ ID NO: 137 - DNA, 21 bp, synthetic construct
  source 1..21: mol_type = other DNA
gcacattggactaaagcagtg

SEQ ID NO: 138 - DNA, 21 bp, synthetic construct
  source 1..21: mol_type = other DNA
actagcccacaggtgatgctg

SEQ ID NO: 139 - DNA, 21 bp, synthetic construct
  source 1..21: mol_type = other DNA
cctcacgtatggtgggtacat

SEQ ID NO: 140 - DNA, 21 bp, synthetic construct
  source 1..21: mol_type = other DNA
gtaactgtgaccaaggacact

SEQ ID NO: 141 - DNA, 21 bp, synthetic construct
  source 1..21: mol_type = other DNA
tgggtatgagccccttgacca

SEQ ID NO: 142 - DNA, 1361 bp, synthetic construct
  source 1..1361: mol_type = other DNA
aggaaataagagagaaaagaagagtaagaagaaatataagagccaccatggcccccaagaagaagcggaaggtgggcatccacggcgtgcccgccgccggcagcagcggatccctggagcctggggagaaaccctacaaatgccctgagtgcgggaagagtttcagccacaagaacgcgttgcagaaccaccaaaggacccatactggagagaaaccgtacaagtgccctgaatgtggaaagtcgttctcccaacgcgccaacctgagagctcaccaacgcactcataccggggaaaagccctataagtgcccggaatgcggcaaaagcttctcccggtccgacaagcttgtgcggcaccagcggactcacactggggagaagccctacaagtgcccagaatgcggaaagagcttcagccaggccggtcatctggcctcgcatcagcggacccacactggcgaaaagccttacaagtgtccggagtgcggaaagtccttctcccggaacgacgcactcaccgaacaccagcgcacccacaccggagagaagccgtataagtgtccagaatgtggcaaatccttctccacatctggcaatctgaccgagcaccagagaacccataccggagaaaagccgtacaaatgtcccgaatgcgggaagtcattttcccgctcggatcacctgaccaaccaccagaggactcacacgggaaagaaaacctcagctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 143 - DNA, 1361 bp, synthetic construct
  source 1..1361: mol_type = other DNA
aggaaataagagagaaaagaagagtaagaagaaatataagagccaccatggcccccaagaagaagcggaaggtgggcatccacggcgtgcccgccgccggcagcagcggatcccttgagcccggcgaaaagccctacaagtgcccggaatgcggaaaatcgttcagctcaaagaaggccttgactgaacaccaacggacccacacgggagaaaaaccgtataagtgtccagagtgcgggaagtcattctccacttcccactccctgaccgagcaccagcgcactcacaccggagagaagccttacaagtgtcctgagtgcggaaagtctttcagccacaagaacgcgctgcagaaccaccagaggacacataccggcgaaaaaccctacaaatgcccggagtgtggaaagtccttctcgcaatcctccaatctggtccggcaccagagaacccatactggggaaaaaccttacaaatgtccggagtgcggcaaaagcttttcccgctccgaccacctcaccactcaccaaagaacccacactggggagaagccctataagtgccctgagtgtgggaagtccttcagcaccaagaactcgctcaccgaacaccagcggactcataccggagaaaagccgtacaagtgcccagaatgtggaaagagcttctcgaccaccggcaacctgactgtgcatcagcgcacccataccggcaaaaagacctcagctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 144 - DNA, 1361 bp, synthetic construct
  source 1..1361: mol_type = other DNA
aggaaataagagagaaaagaagagtaagaagaaatataagagccaccatggcccccaagaagaagcggaaggtgggcatccacggcgtgcccgccgccggcagcagcggatccctggagcccggagaaaaaccctataagtgtccggagtgcggaaagtcgttctccacttccggcaaccttgtgcggcaccagcgaacacataccggagaaaagccctacaagtgccctgagtgtgggaagtccttctcccgggcggacaacttgaccgagcaccagagaacccatactggggaaaagccgtataagtgccccgaatgtggaaagtcctttagccaaagcggcgatctcaggcggcatcagcgcacccacactggcgaaaagccatacaagtgcccggagtgtggaaaatctttctcgcacaagaacgcactgcagaatcaccagcggacccataccggggagaagccgtacaagtgtcctgaatgcggaaagagcttctccgaccctggcaacctcgtgcgccaccaacgcactcacaccggtgaaaagccttacaagtgcccagagtgcgggaagtcattcagccagcgggccaacctgagagcccaccaaagaacccacaccggagagaaaccctacaaatgcccggaatgcggcaaatcgttctccgactgccgcgatctggctcggcaccagaggactcacacgggcaaaaagacctcagctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 145 - DNA, 1361 bp, synthetic construct
  source 1..1361: mol_type = other DNA
aggaaataagagagaaaagaagagtaagaagaaatataagagccaccatggcccccaagaagaagcggaaggtgggcatccacggcgtgcccgccgccggcagcagcggatccctggaaccgggagagaaaccctataagtgtccggagtgcggaaagtctttctcccgcaacgacgcgcttaccgagcaccagcgcacccacactggagaaaaaccctacaaatgccctgaatgtggcaaatcattctccgataagaaggacttgaccagacaccagcggacccacaccggagaaaagccgtataagtgccccgagtgtgggaagtcgttcagccgggccgataacctgacggaacaccagagaactcataccggggagaagccgtacaagtgtcctgaatgcgggaagtcctttagccacaagaacgccctgcaaaaccaccaacggacccataccggagagaagccttacaagtgcccggaatgcggaaagagcttctcgacctcccattcgctgactgagcatcagaggacacacaccggcgaaaagccatacaagtgccctgagtgcggcaaatccttctccacttccggcaatctcgtgcggcaccagaggactcacaccggggaaaagccctacaagtgcccagaatgtggaaagtccttcagccgcgaggacaacctccacacccatcagcgcactcacactggcaaaaagacctcagctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 146 - DNA, 1361 bp, synthetic construct
  source 1..1361: mol_type = other DNA
aggaaataagagagaaaagaagagtaagaagaaatataagagccaccatggcccccaagaagaagcggaaggtgggcatccacggcgtgcccgccgccggcagcagcggatccctggagcctggcgaaaagccttacaagtgtcccgaatgcggcaaatcgttctcacggaaggacaaccttaagaaccaccagaggacccataccggagaaaagccatacaaatgcccggagtgcgggaagtccttctcccgcgccgacaacctgactgagcaccaacggacccatacaggggagaagccctataagtgcccggaatgtggaaagtcgttctcggaccccggtgcactcgtgcgccatcagaggacgcacaccggggaaaagccctacaaatgtccggaatgcggaaagtccttttccgacaagaaggatttgaccagacaccagcgcactcacaccggagagaagccgtacaagtgcccagagtgcggcaaaagcttctccgatcctggagctctggtccggcatcagcggacccacaccggtgaaaagccgtataagtgtccggagtgtggaaagagcttctcccggtccgacaatctcgtgcggcaccaacgcacccatactggcgaaaaaccctacaagtgccctgagtgcggaaaaagcttcagctctcctgccgacctgactagacaccagagaacccacactggcaaaaagacctcagctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 147 - DNA, 1361 bp, synthetic construct
  source 1..1361: mol_type = other DNA
aggaaataagagagaaaagaagagtaagaagaaatataagagccaccatggcccccaagaagaagcggaaggtgggcatccacggcgtgcccgccgccggcagcagcggatccctggaacccggagagaagccgtataagtgtcctgaatgcggaaagtccttctcccgctcggacgagcttgtgcggcaccagcgcactcacactggcgaaaagccgtacaaatgccccgaatgcgggaagtccttcagccaatcgggagatctgcgcagacatcagcggacccacaccggagaaaagccttataagtgccctgagtgcgggaaatcgttctcccaacgggcgaacctcagggcccaccaacggactcacacgggggaaaagccctacaagtgtccggagtgtggaaagtcattctccacccacctggacctgatcaggcaccagcggacacataccggagagaaaccgtacaagtgcccggaatgtggaaagagcttctcaagatccgaccacttgaccacccatcagcgcacccatactggggagaagccttacaaatgtcccgaatgtggcaaatcgttttccacctccggcaacctgaccgagcatcagaggactcacaccggcgaaaaaccatacaagtgcccagagtgcggcaaaagcttcagccagtccggcgatctccggagacaccagcgaacccacactggaaagaaaacctctgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 148 - DNA, 1361 bp, synthetic construct
  source 1..1361: mol_type = other DNA
aggaaataagagagaaaagaagagtaagaagaaatataagagccaccatggcccccaagaagaagcggaaggtgggcatccacggcgtgcccgccgccggcagcagcggatccctggagcctggcgaaaaaccgtacaagtgccccgaatgtggaaagtctttctcccgcaacgacgcgttgaccgaacaccagcggacccataccggtgaaaagccttacaagtgccctgagtgcggcaaaagcttctcccgccgcgatgaactgaacgtgcaccaacgcacccacactggagaaaagccatacaaatgcccggagtgtgggaagtccttctcccggtccgatgagcttgtgcggcaccagagaactcacacaggggaaaagccctataagtgcccagagtgcgggaagtcattcagccgggccgacaacctgaccgagcaccaaaggactcacaccggagagaagccgtataagtgtcctgagtgtggaaagagcttcagcacctcccactccctcaccgaacatcagcggactcatactggggagaagccctacaagtgtcccgaatgcggcaaatccttttcggaacggtcgcacctgagagagcatcagaggacgcacaccggcgaaaagccgtacaaatgtccggagtgcggaaagtcgttctccacccacctcgacctgatcagacaccagcgcacccatactggcaaaaagacctcagctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 149 - DNA, 1361 bp, synthetic construct
  source 1..1361: mol_type = other DNA
aggaaataagagagaaaagaagagtaagaagaaatataagagccaccatggcccccaagaagaagcggaaggtgggcatccacggcgtgcccgccgccggcagcagcggatccctggagcccggagagaaaccgtacaagtgtccagagtgcggcaaatccttctcgacctccggcaacctgactgaacaccagcgcactcacactggagaaaagccgtacaaatgtcctgaatgcggaaagagcttcagccagtcctcctcacttgtgcgccaccagaggacccacaccggagaaaaaccgtataagtgtcctgagtgtgggaagtcattctcaagaagcgatcacctcaccacccatcagcggacccatacgggggagaagccctacaaatgcccggagtgcggaaagtcgttcagccggtccgaccacctgaccacccaccaaagaactcacaccggcgaaaagccttacaagtgccctgaatgtggaaagtccttttcccaatcgtcctccttggtccggcatcagaggactcacacaggagagaagccatacaagtgccccgaatgcgggaagtccttcagctcgaagaaggccctgactgagcaccagcggactcataccggggaaaagccctataagtgcccggaatgcggcaaaagcttctcgaccaagaactccctcaccgagcatcagcgcacccacactggcaaaaagacctctgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 150 - DNA, 1361 bp, synthetic construct
  source 1..1361: mol_type = other DNA
aggaaataagagagaaaagaagagtaagaagaaatataagagccaccatggcccccaagaagaagcggaaggtgggcatccacggcgtgcccgccgccggcagcagcggatccctggaacccggagagaagccctataagtgtccggagtgcggcaaatccttttccacccatctcgacctgattaggcaccagagaactcacactggggagaagccatacaagtgccccgaatgtggaaagtctttctccgatccgggcaaccttgtccggcaccagaggacccacacgggcgaaaaaccttataagtgccctgagtgtgggaagtccttcagccggaaggacaacctgaagaaccaccaaagaacacacaccggagaaaagccctacaaatgcccagagtgcgggaagtcgttctccgacaagaaggatttgacccggcaccaacggacccacactggcgaaaagccgtacaaatgtcccgaatgcggaaagagcttctcgcgctccgacgagctcgtgcgccatcagcggactcataccggagagaaaccgtacaagtgtcctgagtgcggaaagtccttctcaacccacctggacctgatcagacaccagcgcactcacaccggggaaaagccttacaagtgcccggaatgcggcaaaagcttctcccaatcgagctccctcgtgcggcatcagcgcacccataccggaaagaaaacctcagctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 151 - DNA, 1361 bp, synthetic construct
  source 1..1361: mol_type = other DNA
aggaaataagagagaaaagaagagtaagaagaaatataagagccaccatggcccccaagaagaagcggaaggtgggcatccacggcgtgcccgccgccggcagcagcggatccttggaaccgggggaaaagccgtacaagtgccccgaatgtggcaaatcattctctacctcccactcactgaccgagcaccagaggacccacaccggagagaaaccctacaagtgtccagaatgcggaaagtccttctcgcaagccggtcaccttgcgtcccaccaacgcactcatactggagaaaaaccatacaagtgcccggaatgcggcaaaagcttcagcaccaagaacagcctgactgagcatcagaggactcacaccggcgaaaagccctataagtgtcctgagtgcggcaaatccttctccgattgtcgggacctggccagacaccagcgcactcacacaggagaaaagccttataagtgccctgagtgtggaaagtcgtttagccaagctggtcacctggcatcccatcagcggactcataccggggagaagccttacaaatgtccggagtgcgggaagtcgttcagccagtcctcctccctcgtgcggcaccagagaacccacactggagagaagccctacaaatgccctgaatgtggaaagagcttctcccgctcggaccacctcaccacgcaccagcggacccataccggcaaaaagacctcagctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 152 - protein, 362 aa, synthetic construct
MAPKKKRKVGIHGVPAAGSSGSLEPGEKPYKCPECGKSFSHKNALQNHQRTHTGEKPYKCPECGKSFSQRANLRAHQRTHTGEKPYKCPECGKSFSRSDKLVRHQRTHTGEKPYKCPECGKSFSQAGHLASHQRTHTGEKPYKCPECGKSFSRNDALTEHQRTHTGEKPYKCPECGKSFSTSGNLTEHQRTHTGEKPYKCPECGKSFSRSDHLTNHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 153 - protein, 362 aa, synthetic construct
MAPKKKRKVGIHGVPAAGSSGSLEPGEKPYKCPECGKSFSSKKALTEHQRTHTGEKPYKCPECGKSFSTSHSLTEHQRTHTGEKPYKCPECGKSFSHKNALQNHQRTHTGEKPYKCPECGKSFSQSSNLVRHQRTHTGEKPYKCPECGKSFSRSDHLTTHQRTHTGEKPYKCPECGKSFSTKNSLTEHQRTHTGEKPYKCPECGKSFSTTGNLTVHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 154 - protein, 362 aa, synthetic construct
MAPKKKRKVGIHGVPAAGSSGSLEPGEKPYKCPECGKSFSTSGNLVRHQRTHTGEKPYKCPECGKSFSRADNLTEHQRTHTGEKPYKCPECGKSFSQSGDLRRHQRTHTGEKPYKCPECGKSFSHKNALQNHQRTHTGEKPYKCPECGKSFSDPGNLVRHQRTHTGEKPYKCPECGKSFSQRANLRAHQRTHTGEKPYKCPECGKSFSDCRDLARHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 155 - protein, 362 aa, synthetic construct
MAPKKKRKVGIHGVPAAGSSGSLEPGEKPYKCPECGKSFSRNDALTEHQRTHTGEKPYKCPECGKSFSDKKDLTRHQRTHTGEKPYKCPECGKSFSRADNLTEHQRTHTGEKPYKCPECGKSFSHKNALQNHQRTHTGEKPYKCPECGKSFSTSHSLTEHQRTHTGEKPYKCPECGKSFSTSGNLVRHQRTHTGEKPYKCPECGKSFSREDNLHTHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 156 - protein, 362 aa, synthetic construct
MAPKKKRKVGIHGVPAAGSSGSLEPGEKPYKCPECGKSFSRKDNLKNHQRTHTGEKPYKCPECGKSFSRADNLTEHQRTHTGEKPYKCPECGKSFSDPGALVRHQRTHTGEKPYKCPECGKSFSDKKDLTRHQRTHTGEKPYKCPECGKSFSDPGALVRHQRTHTGEKPYKCPECGKSFSRSDNLVRHQRTHTGEKPYKCPECGKSFSSPADLTRHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 157 - protein, 362 aa, synthetic construct
MAPKKKRKVGIHGVPAAGSSGSLEPGEKPYKCPECGKSFSRSDELVRHQRTHTGEKPYKCPECGKSFSQSGDLRRHQRTHTGEKPYKCPECGKSFSQRANLRAHQRTHTGEKPYKCPECGKSFSTHLDLIRHQRTHTGEKPYKCPECGKSFSRSDHLTTHQRTHTGEKPYKCPECGKSFSTSGNLTEHQRTHTGEKPYKCPECGKSFSQSGDLRRHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 158 - protein, 362 aa, synthetic construct
MAPKKKRKVGIHGVPAAGSSGSLEPGEKPYKCPECGKSFSRNDALTEHQRTHTGEKPYKCPECGKSFSRRDELNVHQRTHTGEKPYKCPECGKSFSRSDELVRHQRTHTGEKPYKCPECGKSFSRADNLTEHQRTHTGEKPYKCPECGKSFSTSHSLTEHQRTHTGEKPYKCPECGKSFSERSHLREHQRTHTGEKPYKCPECGKSFSTHLDLIRHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 159 - protein, 362 aa, synthetic construct
MAPKKKRKVGIHGVPAAGSSGSLEPGEKPYKCPECGKSFSTSGNLTEHQRTHTGEKPYKCPECGKSFSQSSSLVRHQRTHTGEKPYKCPECGKSFSRSDHLTTHQRTHTGEKPYKCPECGKSFSRSDHLTTHQRTHTGEKPYKCPECGKSFSQSSSLVRHQRTHTGEKPYKCPECGKSFSSKKALTEHQRTHTGEKPYKCPECGKSFSTKNSLTEHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 160 - protein, 362 aa, synthetic construct
MAPKKKRKVGIHGVPAAGSSGSLEPGEKPYKCPECGKSFSTHLDLIRHQRTHTGEKPYKCPECGKSFSDPGNLVRHQRTHTGEKPYKCPECGKSFSRKDNLKNHQRTHTGEKPYKCPECGKSFSDKKDLTRHQRTHTGEKPYKCPECGKSFSRSDELVRHQRTHTGEKPYKCPECGKSFSTHLDLIRHQRTHTGEKPYKCPECGKSFSQSSSLVRHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 161 - protein, 362 aa, synthetic construct
MAPKKKRKVGIHGVPAAGSSGSLEPGEKPYKCPECGKSFSTSHSLTEHQRTHTGEKPYKCPECGKSFSQAGHLASHQRTHTGEKPYKCPECGKSFSTKNSLTEHQRTHTGEKPYKCPECGKSFSDCRDLARHQRTHTGEKPYKCPECGKSFSQAGHLASHQRTHTGEKPYKCPECGKSFSQSSSLVRHQRTHTGEKPYKCPECGKSFSRSDHLTTHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 162 - DNA, 1601 bp, synthetic construct
  source 1..1601: mol_type = other DNA
aatggttttcctgaatttagtagatttaggtttagattggacatgtcatgtgtgtgaaagcagatgtggttaatagggtagtggatagaacagaattcttcccagaaagagcttctattaggtcatattcaaaggaacttttcttcccacgataccatatttgcgttgacacaaaatttcaccttttttccttcttaccctcttccagaaacacaaataatttttatgtgaagaaaatgtttaccaaaattctaaggtaaaccttatatagcttcctgtatgtcattttgtggctcctaatggcaaagttaaggagagaagtttctgcaattgtgacttatctacagataaacaatgccttattattattattcttgaggcatctgtgagggaaaattcaataaggaaaggggattcaaagggagattttaaaaaatccttgggaaattccacacatggtcaaggggctcatacccatgactcctgaggcatgtcagccaaagacattgcacaggattttatactgagtcacctctctggaatctgagatctgaatggagcctggagaaggaagttgagtgtccttggtcacagttaccacttacttgctgagccagaagaattctggaagagttcctctcttcaggagattttttaatgtacccaccatacgtgaggattttaggatccaattcagaccctgttacacagaggtcaccgtccagaagggagacacaagaaatcacttatgatacagcacattggactaaagcagtgattcttaagttctggtgcctggacaagcatcatcagcatcacctgtgggctagttggaaaagacaattttggaggcctccttttcccttctgaaacagacatggtgggagtggagtccagtgagctgtgtttcagcaacccttccaggggattctgacatactgtaaagtctgagaacccactgggctagaaggaagcctcctggcatgaggagagagttgttctggaggccttaaataacatgggagtaggttgaggacaaggttgagaaggtggtggtgcctagaccttttaaaggattaataagattcttgagaaaaaaaaaagccagcagagggaagaagaatggaaaagctaaagctgagggtgacagggaggaagatagacccagagaagagtctggagaggccagaaggggccaagtggggagagaattttatcagggctgggagtataaactgtattatgaagaccatggcactgggataaaggctgaaggctaaagtctgggaaagacacactcagatttgtagaaaagctataaaatttcagattttagaaagctcacttggcagatgtgagtgaaaagtggaggggaggaagctcaaagttgaagcagttaggagatttctatggtgatctcgggccagggaaagcaggactcacctagcacagggggctaggaaggggaagggtctcaaacaggagaacagctgagaaaagggagttggtagaacctcatgttataatacatcacagaagccaaaaatcatgaggtagtatactgactggcagcaaatgcctatgagattggaaagtgagat

SEQ ID NO: 163 - DNA, 962 bp, synthetic construct
  source 1..962: mol_type = other DNA
gttaggttttatttcaccaagtttataaagtaaaccacatatcgttttctcttttgtagatgctgaaagcaaagttcatgtgggaaatgtttggcaatagctgatttatcctcagggtaacaatattctataactcctttgatcttgaggcctctgtgatggaaatgcttggagaaagggattttaaagggagattctgaagtccttgggaaagtccacaagtggacggggcttcatagccatgacaacaaatgacattgtctaggaaacagtgagtcatggcatgctgagcttagaatggagccaacagaaggaacctggcctcggacacagaatcttttggctgctgacccagaatgactgtgaaagactaacactgtttagcagatttttcttgagtgtttactatgtgtgaggttcctgggattcagattcagctactattgttaagaggaaatcaaccaggaagtcagttaagaaaaggtacagtgggttttcaggctgcagggtacagaaatgttcccaggcctggagaacaaaccttcagatcttaatctgtacagggaggtggagggtgaaagaatgatctttcaggaagcgttcaagtagggctgctgcttggattgaattttaaagaatgcataggttatatgcaggatctatatatagatcaatagcttccctgagcacatgttcaaaggttcaaacatttggggtcatttctttgcaagaagagtcactcagtggcctgaaagtccatgcagcaacttccctcatgagagctgcttccgcagcaggcccagggtttctaaaggagagagcacacagatgtaaacactctgtggttctgaggactgtcacctcttcttttcacccatcacttttgtcttaagaactctatgctcaaccctaattctcagtctctatatcaattcccaccaaacagatgcaaagtcctgtcc

SEQ ID NO: 164 - protein, 355 aa, synthetic construct
MAPKKKRKVGGSGGSLEPGEKPYKCPECGKSFSDSGNLRVHQRTHTGEKPYKCPECGKSFSQLAHLRAHQRTHTGEKPYKCPECGKSFSTKNSLTEHQRTHTGEKPYKCPECGKSFSTSGSLVRHQRTHTGEKPYKCPECGKSFSSPADLTRHQRTHTGEKPYKCPECGKSFSQSSSLVRHQRTHTGEKPYKCPECGKSFSQSGDLRRHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 165 - protein, 355 aa, synthetic construct
MAPKKKRKVGGSGGSLEPGEKPYKCPECGKSFSRSDNLVRHQRTHTGEKPYKCPECGKSFSQNSTLTEHQRTHTGEKPYKCPECGKSFSQSSSLVRHQRTHTGEKPYKCPECGKSFSQKSSLIAHQRTHTGEKPYKCPECGKSFSTSGNLVRHQRTHTGEKPYKCPECGKSFSSPADLTRHQRTHTGEKPYKCPECGKSFSRSDELVRHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 166 - protein, 355 aa, synthetic construct
MAPKKKRKVGGSGGSLEPGEKPYKCPECGKSFSTSGSLVRHQRTHTGEKPYKCPECGKSFSQRANLRAHQRTHTGEKPYKCPECGKSFSRTDTLRDHQRTHTGEKPYKCPECGKSFSRRDELNVHQRTHTGEKPYKCPECGKSFSHRTTLTNHQRTHTGEKPYKCPECGKSFSRSDKLTEHQRTHTGEKPYKCPECGKSFSQKSSLIAHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 167 - protein, 355 aa, synthetic construct
MAPKKKRKVGGSGGSLEPGEKPYKCPECGKSFSRADNLTEHQRTHTGEKPYKCPECGKSFSRSDHLTTHQRTHTGEKPYKCPECGKSFSRSDNLVRHQRTHTGEKPYKCPECGKSFSQNSTLTEHQRTHTGEKPYKCPECGKSFSQSSSLVRHQRTHTGEKPYKCPECGKSFSQKSSLIAHQRTHTGEKPYKCPECGKSFSTSGNLVRHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 168 - protein, 355 aa, synthetic construct
MAPKKKRKVGGSGGSLEPGEKPYKCPECGKSFSRSDHLTTHQRTHTGEKPYKCPECGKSFSDPGHLVRHQRTHTGEKPYKCPECGKSFSHKNALQNHQRTHTGEKPYKCPECGKSFSRRDELNVHQRTHTGEKPYKCPECGKSFSQRANLRAHQRTHTGEKPYKCPECGKSFSTTGNLTVHQRTHTGEKPYKCPECGKSFSSKKHLAEHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 169 - protein, 355 aa, synthetic construct
MAPKKKRKVGGSGGSLEPGEKPYKCPECGKSFSSKKALTEHQRTHTGEKPYKCPECGKSFSTSGNLTEHQRTHTGEKPYKCPECGKSFSHRTTLTNHQRTHTGEKPYKCPECGKSFSRNDALTEHQRTHTGEKPYKCPECGKSFSDSGNLRVHQRTHTGEKPYKCPECGKSFSQSGDLRRHQRTHTGEKPYKCPECGKSFSRSDHLTNHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 170 - protein, 204 aa, synthetic construct
LEPGEKPYKCPECGKSFSDSGNLRVHQRTHTGEKPYKCPECGKSFSQLAHLRAHQRTHTGEKPYKCPECGKSFSTKNSLTEHQRTHTGEKPYKCPECGKSFSTSGSLVRHQRTHTGEKPYKCPECGKSFSSPADLTRHQRTHTGEKPYKCPECGKSFSQSSSLVRHQRTHTGEKPYKCPECGKSFSQSGDLRRHQRTHTGKKTS

SEQ ID NO: 171 - protein, 204 aa, synthetic construct
LEPGEKPYKCPECGKSFSRSDNLVRHQRTHTGEKPYKCPECGKSFSQNSTLTEHQRTHTGEKPYKCPECGKSFSQSSSLVRHQRTHTGEKPYKCPECGKSFSQKSSLIAHQRTHTGEKPYKCPECGKSFSTSGNLVRHQRTHTGEKPYKCPECGKSFSSPADLTRHQRTHTGEKPYKCPECGKSFSRSDELVRHQRTHTGKKTS

SEQ ID NO: 172 - protein, 204 aa, synthetic construct
LEPGEKPYKCPECGKSFSTSGSLVRHQRTHTGEKPYKCPECGKSFSQRANLRAHQRTHTGEKPYKCPECGKSFSRTDTLRDHQRTHTGEKPYKCPECGKSFSRRDELNVHQRTHTGEKPYKCPECGKSFSHRTTLTNHQRTHTGEKPYKCPECGKSFSRSDKLTEHQRTHTGEKPYKCPECGKSFSQKSSLIAHQRTHTGKKTS

SEQ ID NO: 173 - protein, 204 aa, synthetic construct
LEPGEKPYKCPECGKSFSRADNLTEHQRTHTGEKPYKCPECGKSFSRSDHLTTHQRTHTGEKPYKCPECGKSFSRSDNLVRHQRTHTGEKPYKCPECGKSFSQNSTLTEHQRTHTGEKPYKCPECGKSFSQSSSLVRHQRTHTGEKPYKCPECGKSFSQKSSLIAHQRTHTGEKPYKCPECGKSFSTSGNLVRHQRTHTGKKTS

SEQ ID NO: 174 - protein, 204 aa, synthetic construct
LEPGEKPYKCPECGKSFSRSDHLTTHQRTHTGEKPYKCPECGKSFSDPGHLVRHQRTHTGEKPYKCPECGKSFSHKNALQNHQRTHTGEKPYKCPECGKSFSRRDELNVHQRTHTGEKPYKCPECGKSFSQRANLRAHQRTHTGEKPYKCPECGKSFSTTGNLTVHQRTHTGEKPYKCPECGKSFSSKKHLAEHQRTHTGKKTS

SEQ ID NO: 175 - protein, 204 aa, synthetic construct
LEPGEKPYKCPECGKSFSSKKALTEHQRTHTGEKPYKCPECGKSFSTSGNLTEHQRTHTGEKPYKCPECGKSFSHRTTLTNHQRTHTGEKPYKCPECGKSFSRNDALTEHQRTHTGEKPYKCPECGKSFSDSGNLRVHQRTHTGEKPYKCPECGKSFSQSGDLRRHQRTHTGEKPYKCPECGKSFSRSDHLTNHQRTHTGKKTS

SEQ ID NO: 176-193 - skipped (18 entries)

SEQ ID NO: 194 - DNA, 612 bp, synthetic construct
  source 1..612: mol_type = other DNA
ctggagcccggggagaagccatataagtgtcccgaatgcggaaagtccttcagcgactccggcaaccttagggtccatcagcgaacccacacgggggagaaaccgtacaaatgcccggagtgtgggaagtccttttcccaactggctcacttgcgcgcgcaccagagaactcatactggagaaaagccctacaagtgtccggaatgtggaaagagcttcagcaccaagaactccctcaccgagcaccagcggacccataccggagaaaaaccttacaagtgccctgaatgcggaaagtccttcagcacgtccggttccctggttcggcaccagaggacccacactggagagaagccttacaaatgtccagagtgcggcaaaagcttctcgagccccgccgatttgacccgccaccaaagaactcacaccggggaaaagccgtacaagtgcccggaatgcggcaaatccttctcacaatcctcctctcttgtccggcaccagcgcacacatactggcgaaaaaccatataagtgcccagaatgcgggaagtcgtttagccagtcgggcgatctgaggagacatcagcgcacccacaccggaaagaaaaccagc

SEQ ID NO: 195 - DNA, 612 bp, synthetic construct
  source 1..612: mol_type = other DNA
ctggagcccggcgaaaagccctacaagtgtccggagtgcggaaagagcttctcccgctccgataaccttgtgcgccatcagcgaacccacacgggggagaaaccgtacaaatgcccggagtgtgggaagtccttttcccaaaacagcaccctcaccgagcaccagcggacccataccggagaaaaaccttacaagtgccctgaatgcggaaagtccttcagccaatccagctccctggttcggcaccagaggacccacactggagagaagccttacaaatgtccagagtgcggcaaaagcttctcgcaaaagtcgtcgttgattgcccaccaaagaactcacaccggggaaaagccgtacaagtgcccggaatgcggcaaatccttctcaacttccggcaaccttgtccggcaccagcgcacacatactggcgaaaaaccatataagtgcccagaatgcgggaagtcgtttagctcccccgctgatctgaccagacatcagcggacacacaccggagagaaaccctataagtgtcccgaatgtggaaaatccttcagccgctcggacgaactggtccgccatcagcggacacacaccggaaagaaaacttca

SEQ ID NO: 196 - DNA, 612 bp, synthetic construct
  source 1..612: mol_type = other DNA
ctggaaccgggagagaagccatacaaatgccccgagtgtggaaagtccttttcgacttccggttcgcttgtgcgccatcagcgaacccacacgggggagaaaccgtacaaatgcccggagtgtgggaagtccttttcccaacgggccaacctgagagcccaccaaaggacgcataccggcgaaaagccctacaagtgtcccgaatgtggaaagagcttctctcgcactgacaccctccgggatcaccagcggacccataccggagaaaaaccttacaagtgccctgaatgcggaaagtccttcagccggagggatgaactgaacgtccaccagaggacccacactggagagaagccttacaaatgtccagagtgcggcaaaagcttctcgcatcgcaccaccttgaccaaccaccaaagaactcacaccggggaaaagccgtacaagtgcccggaatgcggcaaatccttctcacgctccgacaagcttactgaacaccagcgcacacatactggcgaaaaaccatataagtgcccagaatgcgggaagtcgtttagccagaagtccagcctgattgctcaccagcgcacccacaccggaaagaaaacctcc

SEQ ID NO: 197 - DNA, 612 bp, synthetic construct
  source 1..612: mol_type = other DNA
ctggaacctggggagaagccatataagtgccctgagtgtggaaagtccttctcccgggctgacaatctgaccgaacaccagcggactcacactggagagaagccgtacaaatgccccgagtgcggaaagtcgttctcccgctccgaccatctcaccacccatcagagaacccacaccggggaaaagccatacaagtgtcctgaatgcggcaaatcattcagccgctcggacaacctcgtgcggcatcaacgaacccacacgggagagaaaccgtataaatgcccggagtgcggcaaaagctttagccagaactccactctgacggagcaccaacgcacccataccggagaaaagccttacaagtgcccagagtgcggtaaaagcttcagccagtcctcctcactcgtgcgccaccagagaactcatactggcgaaaagccgtataagtgcccggaatgtggaaagagcttcagtcagaagtccagcctgattgcgcaccagaggactcataccggcgaaaaaccctacaaatgtccggaatgcggaaaatcgttctcaacttccggcaaccttgtgcggcaccagcgcacacacaccggcaaaaagacttcg

SEQ ID NO: 198 - DNA, 612 bp, synthetic construct
  source 1..612: mol_type = other DNA
ctcgaacccggagaaaagccctacaaatgcccagagtgcggaaagagcttttcccgctccgatcatctcaccacgcaccagcggacccataccggagaaaaaccttacaagtgccctgaatgcggaaagtccttcagcgaccccggtcacctggttcggcaccagaggacccacactggagagaagccttacaaatgtccagagtgcggcaaaagcttctcgcacaagaacgccttgcaaaaccaccaacgcactcataccggcgaaaagccatacaagtgtccggagtgtgggaagtccttctcgcgacgcgacgaactgaacgtccaccaaagaactcacaccggggaaaagccgtacaagtgcccggaatgcggcaaatccttctcacaacgcgccaaccttagggctcaccagcgcacacatactggcgaaaaaccatataagtgcccagaatgcgggaagtcgtttagcacgactggcaacctgaccgtccatcagcggacacacaccggagagaaaccctataagtgtcccgaatgtggaaaatccttcagctctaagaagcatctggccgagcaccagagaacccacactgggaagaaaaccagc

SEQ ID NO: 199 - DNA, 612 bp, synthetic construct
  source 1..612: mol_type = other DNA
ctggagcctggagagaagccgtataagtgcccagaatgtggaaagtcattcagctccaagaaggctctgaccgaacaccaacggacgcacaccggagaaaaaccctacaaatgccctgaatgcgggaagtccttttccacctccggcaacctgaccgagcaccagcggactcacactggagagaaaccttacaagtgccctgagtgtggaaagagcttctcccatcgcactacccttaccaatcaccaacgcacccataccggcgaaaaaccatacaaatgtccagagtgcggcaaatccttcagccgcaacgatgcgctcaccgagcatcagcgcactcatactggcgaaaagccctataagtgtccggaatgcggaaagtcgttcagcgactccggaaaccttagagtgcaccagaggacacacactggggaaaagccgtacaagtgtcccgaatgcggcaaaagcttcagccagtccggggatctgcggaggcaccagcgaacccatacgggagaaaagccttataaatgcccggagtgcggaaagagtttctcgcggagcgaccacctgaccaaccaccagagaacccacactggcaaaaagacctcg

SEQ ID NO: 200 - skipped

SEQ ID NO: 201 - DNA, 7433 bp, synthetic construct
  source 1..7433: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggtacctaatacgactcactataaggaaataagagagaaaagaagagtaagaagaaatataagagccaccatggcccccaagaagaagcggaaggtgggcatccacggcgtgcccgccgccgccaagcggaactacatcctgggcctggccatcggcatcaccagcgtgggctacggcatcatcgactacgagacccgggacgtgatcgacgccggcgtgcggctgttcaaggaggccaacgtggagaacaacgagggccggcggagcaagcggggcgcccggcggctgaagcggcggcggcggcaccggatccagcgggtgaagaagctgctgttcgactacaacctgctgaccgaccacagcgagctgagcggcatcaacccctacgaggcccgggtgaagggcctgagccagaagctgagcgaggaggagttcagcgccgccctgctgcacctggccaagcggcggggcgtgcacaacgtgaacgaggtggaggaggacaccggcaacgagctgagcaccaaggagcagatcagccggaacagcaaggccctggaggagaagtacgtggccgagctgcagctggagcggctgaagaaggacggcgaggtgcggggcagcatcaaccggttcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcacccgggacgagaacgagaagctggagtactacgagaagttccagatcatcgagaacgtgttcaagcagaagaagaagcccaccctgaagcagatcgccaaggagatcctggtgaacgaggaggacatcaagggctaccgggtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaacagcgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgagccagcagaaggagatccccaccaccctggtggacgacttcatcctgagccccgtggtgaagcggagcttcatccagagcatcaaggtgatcaacgccatcatcaagaagtacggcctgcccaacgacatcatcatcgagctggcccgggagaagaacagcaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggacgccatcatcccccggagcgtgagcttcgacaacagcttcaacaacaaggtgctggtgaagcaggaggagaacagcaagaagggcaaccggacccccttccagtacctgagcagcagcgacagcaagatcagctacgagaccttcaagaagcacatcctgaacctggccaagggcaagggccggatcagcaagaccaagaaggagtacctgctggaggagcgggacatcaaccggttcagcgtgcagaaggacttcatcaaccggaacctggtggacacccggtacgccacccggggcctgatgaacctgctgcggagctacttccgggtgaacaacctggacgtgaaggtgaaatccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaaccggaagctgatcaacgacaccctgtacagcacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagccggaacaaggtggtgaagctgagcctgaagccctaccggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtaccgggtgatcggcgtgaacaacgacctgctgaaccggatcgaggtgaacatgatcgacatcacctaccgggagtacctggagaacatgaacgacaagcggcccccccacatcatcaagaccatcgccagcaagacccagagcatcaagaagtacagcaccgacatcctgggcaacctgtacgaggtgaaatccaagaagcacccccagatcatcaagaagggcaagcggcccgccgccaccaagaaggccggccaggccaagaagaagaaggcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaattgtcttcttcatcgcctgcagatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 202 - DNA, 4762 bp, synthetic construct
  source 1..4762: mol_type = other DNA
aggaaataagagagaaaagaagagtaagaagaaatataagagccaccatggcccccaagaagaagcggaaggtgggcatccacggcgtgcccgccgccgccaagcggaactacatcctgggcctggccatcggcatcaccagcgtgggctacggcatcatcgactacgagacccgggacgtgatcgacgccggcgtgcggctgttcaaggaggccaacgtggagaacaacgagggccggcggagcaagcggggcgcccggcggctgaagcggcggcggcggcaccggatccagcgggtgaagaagctgctgttcgactacaacctgctgaccgaccacagcgagctgagcggcatcaacccctacgaggcccgggtgaagggcctgagccagaagctgagcgaggaggagttcagcgccgccctgctgcacctggccaagcggcggggcgtgcacaacgtgaacgaggtggaggaggacaccggcaacgagctgagcaccaaggagcagatcagccggaacagcaaggccctggaggagaagtacgtggccgagctgcagctggagcggctgaagaaggacggcgaggtgcggggcagcatcaaccggttcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcacccgggacgagaacgagaagctggagtactacgagaagttccagatcatcgagaacgtgttcaagcagaagaagaagcccaccctgaagcagatcgccaaggagatcctggtgaacgaggaggacatcaagggctaccgggtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaacagcgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgagccagcagaaggagatccccaccaccctggtggacgacttcatcctgagccccgtggtgaagcggagcttcatccagagcatcaaggtgatcaacgccatcatcaagaagtacggcctgcccaacgacatcatcatcgagctggcccgggagaagaacagcaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggacgccatcatcccccggagcgtgagcttcgacaacagcttcaacaacaaggtgctggtgaagcaggaggagaacagcaagaagggcaaccggacccccttccagtacctgagcagcagcgacagcaagatcagctacgagaccttcaagaagcacatcctgaacctggccaagggcaagggccggatcagcaagaccaagaaggagtacctgctggaggagcgggacatcaaccggttcagcgtgcagaaggacttcatcaaccggaacctggtggacacccggtacgccacccggggcctgatgaacctgctgcggagctacttccgggtgaacaacctggacgtgaaggtgaaatccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaaccggaagctgatcaacgacaccctgtacagcacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagccggaacaaggtggtgaagctgagcctgaagccctaccggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtaccgggtgatcggcgtgaacaacgacctgctgaaccggatcgaggtgaacatgatcgacatcacctaccgggagtacctggagaacatgaacgacaagcggcccccccacatcatcaagaccatcgccagcaagacccagagcatcaagaagtacagcaccgacatcctgggcaacctgtacgaggtgaaatccaagaagcacccccagatcatcaagaagggcaagcggcccgccgccaccaagaaggccggccaggccaagaagaagaaggcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 203 - protein, 1506 aa, synthetic construct
MAPKKKRKVGIHGVPAAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGKRPAATKKAGQAKKKKARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 204 - DNA, 7499 bp, synthetic construct
  source 1..7499: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggtacctaatacgactcactataaggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaaggccagcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaattgtcttcttcatcgcctgcagatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 205 - DNA, 4828 bp, synthetic construct
  source 1..4828: mol_type = other DNA
aggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaaggccagcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 206 - protein, 1528 aa, synthetic construct
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 207 - RNA, 5705 nt, synthetic construct
  source 1..5705: mol_type = other RNA
aaggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaaggcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 208 - protein, 1820 aa, synthetic construct
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 209 - RNA, 6775 nt, synthetic construct
  source 1..6775: mol_type = other RNA
aggaaataagagagaaaagaagagtaagaagaaatataagagccaccatggcccccaagaagaagcggaaggtgggcggcagcggcggcagcggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatccccagcaccggcggcagcggcggcagcggcggcagcggcggcagcggcggcagcggccgacccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 210 - protein, 2177 aa, synthetic construct
MAPKKKRKVGGSGGSGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKGSYPYDVPDYA

SEQ ID NO: 211 - RNA, 6202 nt, synthetic construct
  source 1..6202: mol_type = other RNA
aggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcgccggcggcggcggcagcggcggcggcggcagcggcggcggcggcagcggccccaagaagaagcggaaggtggccgccgccggcagcaaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 212 - protein, 1986 aa, synthetic construct
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSAGGGGSGGGGSGGGGSGPKKKRKVAAAGSNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLSGGKRPAATKKAGQAKKKKGSYPYDVPDYA

SEQ ID NO: 213 - RNA, 5698 nt, synthetic construct
  source 1..5698: mol_type = other RNA
aggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagtcgggcgggggtggctcagaggagcccgaggagcccgccgatagcggacaatctctggtgcccgtctacatctacagccccgaatatgtgagcatgtgtgattccctcgccaagatccctaagagagccagcatggtgcattctctgatcgaggcctacgctctgcataagcaaatgaggatcgtgaagcccaaggtcgccagcatggaagagatggccacctttcacaccgatgcctacctccaacatctccagaaggtgtcccaagagggcgacgacgaccaccccgactccattgagtacggactgggctatgattgccccgccaccgagggcatctttgactatgccgccgctatcggcggagctaccatcacagccgcccagtgtctgattgatggcatgtgcaaggtcgccatcaactggtccggaggctggcatcatgccaagaaggatgaggcctccggcttctgttatctgaatgacgccgtgctgggcattctgagactgaggaggaaattcgagaggattctgtacgtggatctggatctgcatcacggagatggagtcgaagatgccttcagcttcaccagcaaggtgatgacagtctctctgcacaagttctcccccggcttctttcccggaaccggcgacgtgtccgacgtgggactgggcaagggaaggtactacagcgtgaacgtgcccattcaagacggcatccaagacgagaagtactaccagatctgcgagtccgtgctcaaggaggtctaccaagccttcaatcctaaggctgtcgtgctccaactgggagctgataccattgctggcgatcccatgtgcagcttcaatatgacacccgtcggaatcggcaagtgcctcaagtacatcctccagtggcagctcgccaccctcattctcggaggaggcggatacaatctggctaataccgccagatgctggacctatctgaccggcgtgattctgggcaaaacactgagcagcgaaatccccgaccacgagtttttcaccgcttacggccccgactacgtgctggagatcacccccagctgcagacccgatagaaacgaaccccatagaatccagcaaattctgaactatatcaagggcaacctcaagcacgtcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 214 - protein, 1818 aa, synthetic construct
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVVGGGGSGKRPAATKKAGQAKKKKGSYPYDVPDYA

SEQ ID NO: 215 - RNA, 7987 nt, synthetic construct
  source 1..7987: mol_type = other RNA
aggaaataagagagaaaagaagagtaagaagaaatataagagccaccatggcccccaagaagaagcggaaggtgggcggcagcggcggcagcggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatccccagcaccggcggcagcggcggcagcggcggcagcggcggcagcggcggcagcggccgacccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagtcgggcgggggtggctcagaggagcccgaggagcccgccgatagcggacaatctctggtgcccgtctacatctacagccccgaatatgtgagcatgtgtgattccctcgccaagatccctaagagagccagcatggtgcattctctgatcgaggcctacgctctgcataagcaaatgaggatcgtgaagcccaaggtcgccagcatggaagagatggccacctttcacaccgatgcctacctccaacatctccagaaggtgtcccaagagggcgacgacgaccaccccgactccattgagtacggactgggctatgattgccccgccaccgagggcatctttgactatgccgccgctatcggcggagctaccatcacagccgcccagtgtctgattgatggcatgtgcaaggtcgccatcaactggtccggaggctggcatcatgccaagaaggatgaggcctccggcttctgttatctgaatgacgccgtgctgggcattctgagactgaggaggaaattcgagaggattctgtacgtggatctggatctgcatcacggagatggagtcgaagatgccttcagcttcaccagcaaggtgatgacagtctctctgcacaagttctcccccggcttctttcccggaaccggcgacgtgtccgacgtgggactgggcaagggaaggtactacagcgtgaacgtgcccattcaagacggcatccaagacgagaagtactaccagatctgcgagtccgtgctcaaggaggtctaccaagccttcaatcctaaggctgtcgtgctccaactgggagctgataccattgctggcgatcccatgtgcagcttcaatatgacacccgtcggaatcggcaagtgcctcaagtacatcctccagtggcagctcgccaccctcattctcggaggaggcggatacaatctggctaataccgccagatgctggacctatctgaccggcgtgattctgggcaaaacactgagcagcgaaatccccgaccacgagtttttcaccgcttacggccccgactacgtgctggagatcacccccagctgcagacccgatagaaacgaaccccatagaatccagcaaattctgaactatatcaagggcaacctcaagcacgtcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 216 - protein, 2581 aa, synthetic construct
MAPKKKRKVGGSGGSGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVVGGGGSGKRPAATKKAGQAKKKKGSYPYDVPDYA

SEQ ID NO: 217 - RNA, 4486 nt, synthetic construct
  source 1..4486: mol_type = other RNA
aggaaataagagagaaaagaagagtaagaagaaatataagagccaccatggcccccaagaagaagcggaaggtgggcatccacggcgtgcccgccgccgacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 218 - protein, 1414 aa, synthetic construct
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKGSYPYDVPDYA

SEQ ID NO: 219 - DNA, 3262 bp, synthetic construct
  source 1..3262: mol_type = other DNA
aggaaataagagagaaaagaagagtaagaagaaatataagagccaccatggcccccaagaagaagcggaaggtgggcggcagcggcggcagcggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatccccggcagcagcggatccctggagcccggcgaaaagccttacaagtgtcccgagtgcggaaagagcttcagcagagccgataatctgaccgagcaccaaaggacccacaccggagagaagccttataagtgtcccgaatgcggcaaaagcttttctagaagcgatcatctgaccaaccaccagaggacacacaccggagaaaaaccttacaaatgccccgagtgcggcaaaagcttctcccagagcagcaatctggtgagacaccaaaggacccacaccggcgaaaaaccctataaatgccccgaatgtggcaagagctttagcacatccggcgagctggtgaggcatcaaagaacacataccggcgagaagccctacaagtgccccgagtgtggaaaaagcttcagcacccacctcgatctgatcagacaccagaggacccataccggagagaaaccctacaaatgtcccgagtgcggaaagtcctttagccagctggcccatctgagagctcatcaaaggacacacaccggcgagaagccttacaagtgtcccgagtgcggaaaatccttctcccaactggcccatctgagggcccaccagagaacccacaccggcaaaaagacctccgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 220 - DNA, 3262 bp, synthetic construct
  source 1..3262: mol_type = other DNA
aggaaataagagagaaaagaagagtaagaagaaatataagagccaccatggcccccaagaagaagcggaaggtgggcggcagcggcggcagcggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatccccggcagcagcggatccctggagcccggcgaaaagccttacaaatgtcccgaatgcggaaagagcttcagcagagccgacaatctgaccgaacatcagagaacccataccggagaaaaaccttacaaatgtcccgagtgcggcaaaagcttctcccaagccggacatctggccagccaccaaaggacacataccggcgagaaaccctacaagtgccccgagtgcggcaagtccttctctagatccgatgagctggtcagacatcagagaacccataccggcgagaagccttataagtgccccgaatgtggcaagtccttcagccagagagctcatctggagaggcatcaaagaacacacaccggagagaaaccttacaagtgtcccgagtgtggaaagagcttctccagaagggacgagctgaacgtccaccaaagaacccataccggcgaaaagccctataaatgccccgagtgtggaaaatccttttctagatccgaccatctgacaacccaccagaggacccataccggagagaagccctacaaatgccccgagtgtggaaaaagcttctctagaaacgatgctctgacagagcaccaaaggacccacaccggcaaaaagaccagcgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 221 - DNA, 2668 bp, synthetic construct
  source 1..2668: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaagccttacaagtgccccgagtgtggcaaatcctttagcaccaccggaaatctgaccgtccaccagagaacacataccggcgagaaaccctacaagtgtcccgagtgcggcaaatccttcagccagctggcccatctgagagcccatcaaaggacccataccggcgagaaaccttacaagtgtcccgaatgtggaaagtcctttagcagccccgccgatctgacaagacatcaaagaacccacaccggcgagaagccctataaatgtcccgagtgtggaaagtccttcagccagagcggcaatctgaccgagcatcaaagaacccataccggcgaaaagccctataagtgccccgaatgcggaaaaagcttctccacaagcggcgagctggtgagacaccaaaggacacataccggcgaaaagccttataaatgccccgagtgcggcaagagcttctctagaaaggacaatctgaagaaccaccaaagaacacacaccggcgagaagccctacaaatgccccgagtgcggcaagagctttagccagtccagcaacctcgtgagacatcagaggacacataccggaaaaaagaccagcgctagcggcagcggcggcggcagcggcggcaaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 222 - DNA, 3262 bp, synthetic construct
  source 1..3262: mol_type = other DNA
aggaaataagagagaaaagaagagtaagaagaaatataagagccaccatggcccccaagaagaagcggaaggtgggcggcagcggcggcagcggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatccccggcagcagcggatccctggagcccggcgaaaagccttacaagtgccccgagtgtggcaaatcctttagcaccaccggaaatctgaccgtccaccagagaacacataccggcgagaaaccctacaagtgtcccgagtgcggcaaatccttcagccagctggcccatctgagagcccatcaaaggacccataccggcgagaaaccttacaagtgtcccgaatgtggaaagtcctttagcagccccgccgatctgacaagacatcaaagaacccacaccggcgagaagccctataaatgtcccgagtgtggaaagtccttcagccagagcggcaatctgaccgagcatcaaagaacccataccggcgaaaagccctataagtgccccgaatgcggaaaaagcttctccacaagcggcgagctggtgagacaccaaaggacacataccggcgaaaagccttataaatgccccgagtgcggcaagagcttctctagaaaggacaatctgaagaaccaccaaagaacacacaccggcgagaagccctacaaatgccccgagtgcggcaagagctttagccagtccagcaacctcgtgagacatcagaggacacataccggaaaaaagaccagcgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 223 - DNA, 3262 bp, synthetic construct
  source 1..3262: mol_type = other DNA
aggaaataagagagaaaagaagagtaagaagaaatataagagccaccatggcccccaagaagaagcggaaggtgggcggcagcggcggcagcggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatccccggcagcagcggatccctggagcccggcgaaaaaccctataagtgccccgagtgcggcaagagctttagcgatcccggccatctggtgaggcatcagaggacccacaccggcgaaaagccttacaaatgccccgagtgtggaaaaagcttcagcagaagcgatcatctgaccacccatcagaggacacataccggcgagaagccttataaatgccccgaatgtggaaagagcttctccagaagcgaccatctgaccaaccaccagaggacccataccggagaaaaaccttacaaatgccccgagtgtggaaagtccttcagctcccccgccgatctgacaagacatcagagaacccacaccggcgaaaaaccttataaatgtcccgagtgtggcaaaagcttctccgacaagaaggatctgacaagacaccaaaggacccacaccggcgagaaaccttataaatgtcccgaatgcggaaaaagctttagcagaaacgacgctctgaccgaacaccagagaacacataccggagagaaaccctataaatgtcccgagtgcggaaaatccttcagcaccaccggcgctctgacagagcatcagaggacacacaccggcaaaaagacctccgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 224 - DNA, 1897 bp, synthetic construct
  source 1..1897: mol_type = other DNA
aggaaataagagagaaaagaagagtaagaagaaatataagagccaccatggcccccaagaagaagcggaaggtgggcatccacggcgtgcccgccgcctcgggcgggggtggctcaggaaatagggctatcagaaccgagaagatcatctgtagggacgtggctagaggctacgagaacgtgcccattccttgcgtgaatggcgtggatggcgaaccttgccccgaggactacaaatacatctccgagaactgcgaaaccagcacaatgaacatcgacagaaacatcacccacctccagcactgcacatgtgtggatgactgctcctccagcaactgtctgtgcggccagctctccatcagatgctggtacgacaaggacggcagactgctgcaagagttcaacaagatcgaaccccctctcatcttcgagtgtaaccaagcttgcagctgctggagaaactgcaagaatagagtggtccagagcggcatcaaggtgagactgcaactgtacagaaccgccaagatgggatggggagtgagggctctgcaaaccattccccaaggcaccttcatctgcgaatacgtgggcgaactgatctccgacgccgaagctgacgtgagagaggacgacagctatctcttcgatctggacaataaggacggcgaggtgtactgcatcgacgctagatattacggcaacatctctagattcatcaaccacctctgcgatcccaacatcattcccgtgagggtgttcatgctgcaccaagatctgaggttccctagaatcgccttcttcagctctagagacatcagaaccggcgaggagctgggcttcgattacggcgatagattctgggacatcaagtccaagtacttcacatgccagtgcggcagcgagaagtgtaagcacagcgctgaggccattgctctggagcagtctagactggccagactggatggcagcagcggatccctggagcccggagaaaagccttacaaatgccccgagtgcggcaagtccttcagccagctggctcatctgagagctcatcaaaggacccacaccggcgagaagccctataagtgccccgagtgcggaaaatccttctcccagagcagcaatctcgtcagacaccagaggacccacaccggcgagaaaccttacaagtgtcccgaatgtggaaagtccttctcccaaaagagctctctgatcgcccatcagagaacacataccggcgaaaaaccctacaagtgccccgagtgtggcaaaagcttttccaccaccggcaatctgaccgtgcatcaaagaacccacaccggcgaaaaaccctacaaatgccccgagtgtggcaaatccttctccgaccccggccatctggtgaggcaccagaggacacacaccggcgagaaaccttataaatgtcccgaatgcggcaagtcctttagcaccagcggctctctggtgagacatcagaggacacataccggcgaaaagccttacaagtgtcccgagtgtggcaaaagcttcagccagaacagcacactgacagagcatcagagaacccataccggcaaaaagaccagcgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 225 - DNA, 1897 bp, synthetic construct
  source 1..1897: mol_type = other DNA
aggaaataagagagaaaagaagagtaagaagaaatataagagccaccatggcccccaagaagaagcggaaggtgggcatccacggcgtgcccgccgcctcgggcgggggtggctcaggaaatagggctatcagaaccgagaagatcatctgtagggacgtggctagaggctacgagaacgtgcccattccttgcgtgaatggcgtggatggcgaaccttgccccgaggactacaaatacatctccgagaactgcgaaaccagcacaatgaacatcgacagaaacatcacccacctccagcactgcacatgtgtggatgactgctcctccagcaactgtctgtgcggccagctctccatcagatgctggtacgacaaggacggcagactgctgcaagagttcaacaagatcgaaccccctctcatcttcgagtgtaaccaagcttgcagctgctggagaaactgcaagaatagagtggtccagagcggcatcaaggtgagactgcaactgtacagaaccgccaagatgggatggggagtgagggctctgcaaaccattccccaaggcaccttcatctgcgaatacgtgggcgaactgatctccgacgccgaagctgacgtgagagaggacgacagctatctcttcgatctggacaataaggacggcgaggtgtactgcatcgacgctagatattacggcaacatctctagattcatcaaccacctctgcgatcccaacatcattcccgtgagggtgttcatgctgcaccaagatctgaggttccctagaatcgccttcttcagctctagagacatcagaaccggcgaggagctgggcttcgattacggcgatagattctgggacatcaagtccaagtacttcacatgccagtgcggcagcgagaagtgtaagcacagcgctgaggccattgctctggagcagtctagactggccagactggatggcagcagcggatccctggagcccggcgaaaagccttacaagtgccccgagtgtggcaaatcctttagcaccaccggaaatctgaccgtccaccagagaacacataccggcgagaaaccctacaagtgtcccgagtgcggcaaatccttcagccagctggcccatctgagagcccatcaaaggacccataccggcgagaaaccttacaagtgtcccgaatgtggaaagtcctttagcagccccgccgatctgacaagacatcaaagaacccacaccggcgagaagccctataaatgtcccgagtgtggaaagtccttcagccagagcggcaatctgaccgagcatcaaagaacccataccggcgaaaagccctataagtgccccgaatgcggaaaaagcttctccacaagcggcgagctggtgagacaccaaaggacacataccggcgaaaagccttataaatgccccgagtgcggcaagagcttctctagaaaggacaatctgaagaaccaccaaagaacacacaccggcgagaagccctacaaatgccccgagtgcggcaagagctttagccagtccagcaacctcgtgagacatcagaggacacataccggaaaaaagaccagcgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 226 - DNA, 2179 bp, synthetic construct
  source 1..2179: mol_type = other DNA
aggaaataagagagaaaagaagagtaagaagaaatataagagccaccatggcccccaagaagaagcggaaggtgggcatccacggcgtgcccgccgccggcagcagcggatccctggagcccggagaaaagccttacaaatgccccgagtgcggcaagtccttcagccagctggctcatctgagagctcatcaaaggacccacaccggcgagaagccctataagtgccccgagtgcggaaaatccttctcccagagcagcaatctcgtcagacaccagaggacccacaccggcgagaaaccttacaagtgtcccgaatgtggaaagtccttctcccaaaagagctctctgatcgcccatcagagaacacataccggcgaaaaaccctacaagtgccccgagtgtggcaaaagcttttccaccaccggcaatctgaccgtgcatcaaagaacccacaccggcgaaaaaccctacaaatgccccgagtgtggcaaatccttctccgaccccggccatctggtgaggcaccagaggacacacaccggcgagaaaccttataaatgtcccgaatgcggcaagtcctttagcaccagcggctctctggtgagacatcagaggacacataccggcgaaaagccttacaagtgtcccgagtgtggcaaaagcttcagccagaacagcacactgacagagcatcagagaacccataccggcaaaaagaccagcgctagcggcagcggcggcggcagcggcggcgaggagcccgaggagcccgccgatagcggacaatctctggtgcccgtctacatctacagccccgaatatgtgagcatgtgtgattccctcgccaagatccctaagagagccagcatggtgcattctctgatcgaggcctacgctctgcataagcaaatgaggatcgtgaagcccaaggtcgccagcatggaagagatggccacctttcacaccgatgcctacctccaacatctccagaaggtgtcccaagagggcgacgacgaccaccccgactccattgagtacggactgggctatgattgccccgccaccgagggcatctttgactatgccgccgctatcggcggagctaccatcacagccgcccagtgtctgattgatggcatgtgcaaggtcgccatcaactggtccggaggctggcatcatgccaagaaggatgaggcctccggcttctgttatctgaatgacgccgtgctgggcattctgagactgaggaggaaattcgagaggattctgtacgtggatctggatctgcatcacggagatggagtcgaagatgccttcagcttcaccagcaaggtgatgacagtctctctgcacaagttctcccccggcttctttcccggaaccggcgacgtgtccgacgtgggactgggcaagggaaggtactacagcgtgaacgtgcccattcaagacggcatccaagacgagaagtactaccagatctgcgagtccgtgctcaaggaggtctaccaagccttcaatcctaaggctgtcgtgctccaactgggagctgataccattgctggcgatcccatgtgcagcttcaatatgacacccgtcggaatcggcaagtgcctcaagtacatcctccagtggcagctcgccaccctcattctcggaggaggcggatacaatctggctaataccgccagatgctggacctatctgaccggcgtgattctgggcaaaacactgagcagcgaaatccccgaccacgagtttttcaccgcttacggccccgactacgtgctggagatcacccccagctgcagacccgatagaaacgaaccccatagaatccagcaaattctgaactatatcaagggcaacctcaagcacgtcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 227 - DNA, 1897 bp, synthetic construct
  source 1..1897: mol_type = other DNA
aggaaataagagagaaaagaagagtaagaagaaatataagagccaccatggcccccaagaagaagcggaaggtgggcatccacggcgtgcccgccgcctcgggcgggggtggctcaggaaatagggctatcagaaccgagaagatcatctgtagggacgtggctagaggctacgagaacgtgcccattccttgcgtgaatggcgtggatggcgaaccttgccccgaggactacaaatacatctccgagaactgcgaaaccagcacaatgaacatcgacagaaacatcacccacctccagcactgcacatgtgtggatgactgctcctccagcaactgtctgtgcggccagctctccatcagatgctggtacgacaaggacggcagactgctgcaagagttcaacaagatcgaaccccctctcatcttcgagtgtaaccaagcttgcagctgctggagaaactgcaagaatagagtggtccagagcggcatcaaggtgagactgcaactgtacagaaccgccaagatgggatggggagtgagggctctgcaaaccattccccaaggcaccttcatctgcgaatacgtgggcgaactgatctccgacgccgaagctgacgtgagagaggacgacagctatctcttcgatctggacaataaggacggcgaggtgtactgcatcgacgctagatattacggcaacatctctagattcatcaaccacctctgcgatcccaacatcattcccgtgagggtgttcatgctgcaccaagatctgaggttccctagaatcgccttcttcagctctagagacatcagaaccggcgaggagctgggcttcgattacggcgatagattctgggacatcaagtccaagtacttcacatgccagtgcggcagcgagaagtgtaagcacagcgctgaggccattgctctggagcagtctagactggccagactggatggcagcagcggatccctggagcccggcgaaaagccttacaagtgtcccgagtgcggaaagagcttcagcagagccgataatctgaccgagcaccaaaggacccacaccggagagaagccttataagtgtcccgaatgcggcaaaagcttttctagaagcgatcatctgaccaaccaccagaggacacacaccggagaaaaaccttacaaatgccccgagtgcggcaaaagcttctcccagagcagcaatctggtgagacaccaaaggacccacaccggcgaaaaaccctataaatgccccgaatgtggcaagagctttagcacatccggcgagctggtgaggcatcaaagaacacataccggcgagaagccctacaagtgccccgagtgtggaaaaagcttcagcacccacctcgatctgatcagacaccagaggacccataccggagagaaaccctacaaatgtcccgagtgcggaaagtcctttagccagctggcccatctgagagctcatcaaaggacacacaccggcgagaagccttacaagtgtcccgagtgcggaaaatccttctcccaactggcccatctgagggcccaccagagaacccacaccggcaaaaagacctccgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 228 - DNA, 1897 bp, synthetic construct
  source 1..1897: mol_type = other DNA
aggaaataagagagaaaagaagagtaagaagaaatataagagccaccatggcccccaagaagaagcggaaggtgggcatccacggcgtgcccgccgcctcgggcgggggtggctcaggaaatagggctatcagaaccgagaagatcatctgtagggacgtggctagaggctacgagaacgtgcccattccttgcgtgaatggcgtggatggcgaaccttgccccgaggactacaaatacatctccgagaactgcgaaaccagcacaatgaacatcgacagaaacatcacccacctccagcactgcacatgtgtggatgactgctcctccagcaactgtctgtgcggccagctctccatcagatgctggtacgacaaggacggcagactgctgcaagagttcaacaagatcgaaccccctctcatcttcgagtgtaaccaagcttgcagctgctggagaaactgcaagaatagagtggtccagagcggcatcaaggtgagactgcaactgtacagaaccgccaagatgggatggggagtgagggctctgcaaaccattccccaaggcaccttcatctgcgaatacgtgggcgaactgatctccgacgccgaagctgacgtgagagaggacgacagctatctcttcgatctggacaataaggacggcgaggtgtactgcatcgacgctagatattacggcaacatctctagattcatcaaccacctctgcgatcccaacatcattcccgtgagggtgttcatgctgcaccaagatctgaggttccctagaatcgccttcttcagctctagagacatcagaaccggcgaggagctgggcttcgattacggcgatagattctgggacatcaagtccaagtacttcacatgccagtgcggcagcgagaagtgtaagcacagcgctgaggccattgctctggagcagtctagactggccagactggatggcagcagcggatccctggagcccggcgaaaaaccctataaatgccccgagtgtggcaagagcttttccgaccccggacacctcgtgaggcatcagagaacacataccggcgagaaaccctacaagtgccccgaatgcggcaaatccttctctagaaaggacaatctgaaaaaccatcaaagaacccataccggcgagaagccctataaatgtcccgagtgtggaaagagcttcagccacaagaacgctctgcagaaccatcagaggacccataccggcgaaaagccttataagtgccccgagtgcggaaaatccttttctagaagggacgagctgaatgtgcaccaaaggacacataccggagagaaaccctacaaatgccccgagtgcggcaagtccttcagcacctccggcaatctggtgaggcaccaaaggacacacaccggcgaaaaaccttacaagtgtcccgagtgcggaaaaagcttttcccagaacagcacactgaccgaacaccaaaggacccacaccggagagaaaccttataaatgtcccgagtgtggaaagtcctttagccagtccggcaatctgacagagcatcaaagaacccacaccggcaaaaagacctccgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 229 - DNA, 1897 bp, synthetic construct
  source 1..1897: mol_type = other DNA
aggaaataagagagaaaagaagagtaagaagaaatataagagccaccatggcccccaagaagaagcggaaggtgggcatccacggcgtgcccgccgcctcgggcgggggtggctcaggaaatagggctatcagaaccgagaagatcatctgtagggacgtggctagaggctacgagaacgtgcccattccttgcgtgaatggcgtggatggcgaaccttgccccgaggactacaaatacatctccgagaactgcgaaaccagcacaatgaacatcgacagaaacatcacccacctccagcactgcacatgtgtggatgactgctcctccagcaactgtctgtgcggccagctctccatcagatgctggtacgacaaggacggcagactgctgcaagagttcaacaagatcgaaccccctctcatcttcgagtgtaaccaagcttgcagctgctggagaaactgcaagaatagagtggtccagagcggcatcaaggtgagactgcaactgtacagaaccgccaagatgggatggggagtgagggctctgcaaaccattccccaaggcaccttcatctgcgaatacgtgggcgaactgatctccgacgccgaagctgacgtgagagaggacgacagctatctcttcgatctggacaataaggacggcgaggtgtactgcatcgacgctagatattacggcaacatctctagattcatcaaccacctctgcgatcccaacatcattcccgtgagggtgttcatgctgcaccaagatctgaggttccctagaatcgccttcttcagctctagagacatcagaaccggcgaggagctgggcttcgattacggcgatagattctgggacatcaagtccaagtacttcacatgccagtgcggcagcgagaagtgtaagcacagcgctgaggccattgctctggagcagtctagactggccagactggatggcagcagcggatccctggagcccggcgaaaaaccctataagtgccccgagtgcggcaagagctttagcgatcccggccatctggtgaggcatcagaggacccacaccggcgaaaagccttacaaatgccccgagtgtggaaaaagcttcagcagaagcgatcatctgaccacccatcagaggacacataccggcgagaagccttataaatgccccgaatgtggaaagagcttctccagaagcgaccatctgaccaaccaccagaggacccataccggagaaaaaccttacaaatgccccgagtgtggaaagtccttcagctcccccgccgatctgacaagacatcagagaacccacaccggcgaaaaaccttataaatgtcccgagtgtggcaaaagcttctccgacaagaaggatctgacaagacaccaaaggacccacaccggcgagaaaccttataaatgtcccgaatgcggaaaaagctttagcagaaacgacgctctgaccgaacaccagagaacacataccggagagaaaccctataaatgtcccgagtgcggaaaatccttcagcaccaccggcgctctgacagagcatcagaggacacacaccggcaaaaagacctccgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 230 - DNA, 1897 bp, synthetic construct
  source 1..1897: mol_type = other DNA
aggaaataagagagaaaagaagagtaagaagaaatataagagccaccatggcccccaagaagaagcggaaggtgggcatccacggcgtgcccgccgcctcgggcgggggtggctcaggaaatagggctatcagaaccgagaagatcatctgtagggacgtggctagaggctacgagaacgtgcccattccttgcgtgaatggcgtggatggcgaaccttgccccgaggactacaaatacatctccgagaactgcgaaaccagcacaatgaacatcgacagaaacatcacccacctccagcactgcacatgtgtggatgactgctcctccagcaactgtctgtgcggccagctctccatcagatgctggtacgacaaggacggcagactgctgcaagagttcaacaagatcgaaccccctctcatcttcgagtgtaaccaagcttgcagctgctggagaaactgcaagaatagagtggtccagagcggcatcaaggtgagactgcaactgtacagaaccgccaagatgggatggggagtgagggctctgcaaaccattccccaaggcaccttcatctgcgaatacgtgggcgaactgatctccgacgccgaagctgacgtgagagaggacgacagctatctcttcgatctggacaataaggacggcgaggtgtactgcatcgacgctagatattacggcaacatctctagattcatcaaccacctctgcgatcccaacatcattcccgtgagggtgttcatgctgcaccaagatctgaggttccctagaatcgccttcttcagctctagagacatcagaaccggcgaggagctgggcttcgattacggcgatagattctgggacatcaagtccaagtacttcacatgccagtgcggcagcgagaagtgtaagcacagcgctgaggccattgctctggagcagtctagactggccagactggatggcagcagcggatccctggagcccggcgaaaagccttacaaatgtcccgaatgcggaaagagcttcagcagagccgacaatctgaccgaacatcagagaacccataccggagaaaaaccttacaaatgtcccgagtgcggcaaaagcttctcccaagccggacatctggccagccaccaaaggacacataccggcgagaaaccctacaagtgccccgagtgcggcaagtccttctctagatccgatgagctggtcagacatcagagaacccataccggcgagaagccttataagtgccccgaatgtggcaagtccttcagccagagagctcatctggagaggcatcaaagaacacacaccggagagaaaccttacaagtgtcccgagtgtggaaagagcttctccagaagggacgagctgaacgtccaccaaagaacccataccggcgaaaagccctataaatgccccgagtgtggaaaatccttttctagatccgaccatctgacaacccaccagaggacccataccggagagaagccctacaaatgccccgagtgtggaaaaagcttctctagaaacgatgctctgacagagcaccaaaggacccacaccggcaaaaagaccagcgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 231 - DNA, 2668 bp, synthetic construct
  source 1..2668: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaaaccctataagtgccccgagtgcggcaagagctttagcgatcccggccatctggtgaggcatcagaggacccacaccggcgaaaagccttacaaatgccccgagtgtggaaaaagcttcagcagaagcgatcatctgaccacccatcagaggacacataccggcgagaagccttataaatgccccgaatgtggaaagagcttctccagaagcgaccatctgaccaaccaccagaggacccataccggagaaaaaccttacaaatgccccgagtgtggaaagtccttcagctcccccgccgatctgacaagacatcagagaacccacaccggcgaaaaaccttataaatgtcccgagtgtggcaaaagcttctccgacaagaaggatctgacaagacaccaaaggacccacaccggcgagaaaccttataaatgtcccgaatgcggaaaaagctttagcagaaacgacgctctgaccgaacaccagagaacacataccggagagaaaccctataaatgtcccgagtgcggaaaatccttcagcaccaccggcgctctgacagagcatcagaggacacacaccggcaaaaagacctccgctagcggcagcggcggcggcagcggcggcaaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 232 - DNA, 2179 bp, synthetic construct
  source 1..2179: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaagccttacaagtgtcccgagtgcggaaagagcttcagcagagccgataatctgaccgagcaccaaaggacccacaccggagagaagccttataagtgtcccgaatgcggcaaaagcttttctagaagcgatcatctgaccaaccaccagaggacacacaccggagaaaaaccttacaaatgccccgagtgcggcaaaagcttctcccagagcagcaatctggtgagacaccaaaggacccacaccggcgaaaaaccctataaatgccccgaatgtggcaagagctttagcacatccggcgagctggtgaggcatcaaagaacacataccggcgagaagccctacaagtgccccgagtgtggaaaaagcttcagcacccacctcgatctgatcagacaccagaggacccataccggagagaaaccctacaaatgtcccgagtgcggaaagtcctttagccagctggcccatctgagagctcatcaaaggacacacaccggcgagaagccttacaagtgtcccgagtgcggaaaatccttctcccaactggcccatctgagggcccaccagagaacccacaccggcaaaaagacctccgctagcggcagcggcggcggcagcggcggcgaggagcccgaggagcccgccgatagcggacaatctctggtgcccgtctacatctacagccccgaatatgtgagcatgtgtgattccctcgccaagatccctaagagagccagcatggtgcattctctgatcgaggcctacgctctgcataagcaaatgaggatcgtgaagcccaaggtcgccagcatggaagagatggccacctttcacaccgatgcctacctccaacatctccagaaggtgtcccaagagggcgacgacgaccaccccgactccattgagtacggactgggctatgattgccccgccaccgagggcatctttgactatgccgccgctatcggcggagctaccatcacagccgcccagtgtctgattgatggcatgtgcaaggtcgccatcaactggtccggaggctggcatcatgccaagaaggatgaggcctccggcttctgttatctgaatgacgccgtgctgggcattctgagactgaggaggaaattcgagaggattctgtacgtggatctggatctgcatcacggagatggagtcgaagatgccttcagcttcaccagcaaggtgatgacagtctctctgcacaagttctcccccggcttctttcccggaaccggcgacgtgtccgacgtgggactgggcaagggaaggtactacagcgtgaacgtgcccattcaagacggcatccaagacgagaagtactaccagatctgcgagtccgtgctcaaggaggtctaccaagccttcaatcctaaggctgtcgtgctccaactgggagctgataccattgctggcgatcccatgtgcagcttcaatatgacacccgtcggaatcggcaagtgcctcaagtacatcctccagtggcagctcgccaccctcattctcggaggaggcggatacaatctggctaataccgccagatgctggacctatctgaccggcgtgattctgggcaaaacactgagcagcgaaatccccgaccacgagtttttcaccgcttacggccccgactacgtgctggagatcacccccagctgcagacccgatagaaacgaaccccatagaatccagcaaattctgaactatatcaagggcaacctcaagcacgtcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 233 - DNA, 3262 bp, synthetic construct
  source 1..3262: mol_type = other DNA
aggaaataagagagaaaagaagagtaagaagaaatataagagccaccatggcccccaagaagaagcggaaggtgggcggcagcggcggcagcggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatccccggcagcagcggatccctggagcccggagaaaagccttacaaatgccccgagtgcggcaagtccttcagccagctggctcatctgagagctcatcaaaggacccacaccggcgagaagccctataagtgccccgagtgcggaaaatccttctcccagagcagcaatctcgtcagacaccagaggacccacaccggcgagaaaccttacaagtgtcccgaatgtggaaagtccttctcccaaaagagctctctgatcgcccatcagagaacacataccggcgaaaaaccctacaagtgccccgagtgtggcaaaagcttttccaccaccggcaatctgaccgtgcatcaaagaacccacaccggcgaaaaaccctacaaatgccccgagtgtggcaaatccttctccgaccccggccatctggtgaggcaccagaggacacacaccggcgagaaaccttataaatgtcccgaatgcggcaagtcctttagcaccagcggctctctggtgagacatcagaggacacataccggcgaaaagccttacaagtgtcccgagtgtggcaaaagcttcagccagaacagcacactgacagagcatcagagaacccataccggcaaaaagaccagcgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 234 - DNA, 3262 bp, synthetic construct
  source 1..3262: mol_type = other DNA
aggaaataagagagaaaagaagagtaagaagaaatataagagccaccatggcccccaagaagaagcggaaggtgggcggcagcggcggcagcggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatccccggcagcagcggatccctggagcccggcgaaaaaccctataaatgccccgagtgtggcaagagcttttccgaccccggacatctcgtgaggcatcagagaacacataccggcgagaaaccctacaagtgccccgaatgcggcaaatccttctctagaaaggacaatctgaaaaaccatcaaagaacccataccggcgagaagccctataaatgtcccgagtgtggaaagagcttcagccacaagaacgctctgcagaaccatcagaggacccataccggcgaaaagccttataagtgccccgagtgcggaaaatccttttctagaagggacgagctgaatgtgcaccaaaggacacataccggagagaaaccctacaaatgccccgagtgcggcaagtccttcagcacctccggcaatctggtgaggcaccaaaggacacacaccggcgaaaaaccttacaagtgtcccgagtgcggaaaaagcttttcccagaacagcacactgaccgaacaccaaaggacccacaccggagagaaaccttataaatgtcccgagtgtggaaagtcctttagccagtccggcaatctgacagagcatcaaagaacccacaccggcaaaaagacctccgctagcggcagcggcggcggcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 235 - DNA, 2179 bp, synthetic construct
  source 1..2179: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaaaccctataaatgccccgagtgtggcaagagcttttccgaccccggacacctcgtgaggcatcagagaacacataccggcgagaaaccctacaagtgccccgaatgcggcaaatccttctctagaaaggacaatctgaaaaaccatcaaagaacccataccggcgagaagccctataaatgtcccgagtgtggaaagagcttcagccacaagaacgctctgcagaaccatcagaggacccataccggcgaaaagccttataagtgccccgagtgcggaaaatccttttctagaagggacgagctgaatgtgcaccaaaggacacataccggagagaaaccctacaaatgccccgagtgcggcaagtccttcagcacctccggcaatctggtgaggcaccaaaggacacacaccggcgaaaaaccttacaagtgtcccgagtgcggaaaaagcttttcccagaacagcacactgaccgaacaccaaaggacccacaccggagagaaaccttataaatgtcccgagtgtggaaagtcctttagccagtccggcaatctgacagagcatcaaagaacccacaccggcaaaaagacctccgctagcggcagcggcggcggcagcggcggcgaggagcccgaggagcccgccgatagcggacaatctctggtgcccgtctacatctacagccccgaatatgtgagcatgtgtgattccctcgccaagatccctaagagagccagcatggtgcattctctgatcgaggcctacgctctgcataagcaaatgaggatcgtgaagcccaaggtcgccagcatggaagagatggccacctttcacaccgatgcctacctccaacatctccagaaggtgtcccaagagggcgacgacgaccaccccgactccattgagtacggactgggctatgattgccccgccaccgagggcatctttgactatgccgccgctatcggcggagctaccatcacagccgcccagtgtctgattgatggcatgtgcaaggtcgccatcaactggtccggaggctggcatcatgccaagaaggatgaggcctccggcttctgttatctgaatgacgccgtgctgggcattctgagactgaggaggaaattcgagaggattctgtacgtggatctggatctgcatcacggagatggagtcgaagatgccttcagcttcaccagcaaggtgatgacagtctctctgcacaagttctcccccggcttctttcccggaaccggcgacgtgtccgacgtgggactgggcaagggaaggtactacagcgtgaacgtgcccattcaagacggcatccaagacgagaagtactaccagatctgcgagtccgtgctcaaggaggtctaccaagccttcaatcctaaggctgtcgtgctccaactgggagctgataccattgctggcgatcccatgtgcagcttcaatatgacacccgtcggaatcggcaagtgcctcaagtacatcctccagtggcagctcgccaccctcattctcggaggaggcggatacaatctggctaataccgccagatgctggacctatctgaccggcgtgattctgggcaaaacactgagcagcgaaatccccgaccacgagtttttcaccgcttacggccccgactacgtgctggagatcacccccagctgcagacccgatagaaacgaaccccatagaatccagcaaattctgaactatatcaagggcaacctcaagcacgtcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 236 - DNA, 2668 bp, synthetic construct
  source 1..2668: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaagccttacaagtgtcccgagtgcggaaagagcttcagcagagccgataatctgaccgagcaccaaaggacccacaccggagagaagccttataagtgtcccgaatgcggcaaaagcttttctagaagcgatcatctgaccaaccaccagaggacacacaccggagaaaaaccttacaaatgccccgagtgcggcaaaagcttctcccagagcagcaatctggtgagacaccaaaggacccacaccggcgaaaaaccctataaatgccccgaatgtggcaagagctttagcacatccggcgagctggtgaggcatcaaagaacacataccggcgagaagccctacaagtgccccgagtgtggaaaaagcttcagcacccacctcgatctgatcagacaccagaggacccataccggagagaaaccctacaaatgtcccgagtgcggaaagtcctttagccagctggcccatctgagagctcatcaaaggacacacaccggcgagaagccttacaagtgtcccgagtgcggaaaatccttctcccaactggcccatctgagggcccaccagagaacccacaccggcaaaaagacctccgctagcggcagcggcggcggcagcggcggcaaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 237 - DNA, 2668 bp, synthetic construct
  source 1..2668: mol_type = other DNA
aggaaataagagagaaaagaagagtaagaagaaatataagagccaccatggcccccaagaagaagcggaaggtgggcatccacggcgtgcccgccgccggcagcagcggatccctggagcccggagaaaagccttacaaatgccccgagtgcggcaagtccttcagccagctggctcatctgagagctcatcaaaggacccacaccggcgagaagccctataagtgccccgagtgcggaaaatccttctcccagagcagcaatctcgtcagacaccagaggacccacaccggcgagaaaccttacaagtgtcccgaatgtggaaagtccttctcccaaaagagctctctgatcgcccatcagagaacacataccggcgaaaaaccctacaagtgccccgagtgtggcaaaagcttttccaccaccggcaatctgaccgtgcatcaaagaacccacaccggcgaaaaaccctacaaatgccccgagtgtggcaaatccttctccgaccccggccatctggtgaggcaccagaggacacacaccggcgagaaaccttataaatgtcccgaatgcggcaagtcctttagcaccagcggctctctggtgagacatcagaggacacataccggcgaaaagccttacaagtgtcccgagtgtggcaaaagcttcagccagaacagcacactgacagagcatcagagaacccataccggcaaaaagaccagcgctagcggcagcggcggcggcagcggcggcaaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 238 - DNA, 2668 bp, synthetic construct
  source 1..2668: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaaaccctataaatgccccgagtgtggcaagagcttttccgaccccggacacctcgtgaggcatcagagaacacataccggcgagaaaccctacaagtgccccgaatgcggcaaatccttctctagaaaggacaatctgaaaaaccatcaaagaacccataccggcgagaagccctataaatgtcccgagtgtggaaagagcttcagccacaagaacgctctgcagaaccatcagaggacccataccggcgaaaagccttataagtgccccgagtgcggaaaatccttttctagaagggacgagctgaatgtgcaccaaaggacacataccggagagaaaccctacaaatgccccgagtgcggcaagtccttcagcacctccggcaatctggtgaggcaccaaaggacacacaccggcgaaaaaccttacaagtgtcccgagtgcggaaaaagcttttcccagaacagcacactgaccgaacaccaaaggacccacaccggagagaaaccttataaatgtcccgagtgtggaaagtcctttagccagtccggcaatctgacagagcatcaaagaacccacaccggcaaaaagacctccgctagcggcagcggcggcggcagcggcggcaaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 239 - DNA, 2668 bp, synthetic construct
  source 1..2668: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaagccttacaaatgtcccgaatgcggaaagagcttcagcagagccgacaatctgaccgaacatcagagaacccataccggagaaaaaccttacaaatgtcccgagtgcggcaaaagcttctcccaagccggacatctggccagccaccaaaggacacataccggcgagaaaccctacaagtgccccgagtgcggcaagtccttctctagatccgatgagctggtcagacatcagagaacccataccggcgagaagccttataagtgccccgaatgtggcaagtccttcagccagagagctcatctggagaggcatcaaagaacacacaccggagagaaaccttacaagtgtcccgagtgtggaaagagcttctccagaagggacgagctgaacgtccaccaaagaacccataccggcgaaaagccctataaatgccccgagtgtggaaaatccttttctagatccgaccatctgacaacccaccagaggacccataccggagagaagccctacaaatgccccgagtgtggaaaaagcttctctagaaacgatgctctgacagagcaccaaaggacccacaccggcaaaaagaccagcgctagcggcagcggcggcggcagcggcggcaaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 240 - DNA, 2179 bp, synthetic construct
  source 1..2179: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaagccttacaagtgccccgagtgtggcaaatcctttagcaccaccggaaatctgaccgtccaccagagaacacataccggcgagaaaccctacaagtgtcccgagtgcggcaaatccttcagccagctggcccatctgagagcccatcaaaggacccataccggcgagaaaccttacaagtgtcccgaatgtggaaagtcctttagcagccccgccgatctgacaagacatcaaagaacccacaccggcgagaagccctataaatgtcccgagtgtggaaagtccttcagccagagcggcaatctgaccgagcatcaaagaacccataccggcgaaaagccctataagtgccccgaatgcggaaaaagcttctccacaagcggcgagctggtgagacaccaaaggacacataccggcgaaaagccttataaatgccccgagtgcggcaagagcttctctagaaaggacaatctgaagaaccaccaaagaacacacaccggcgagaagccctacaaatgccccgagtgcggcaagagctttagccagtccagcaacctcgtgagacatcagaggacacataccggaaaaaagaccagcgctagcggcagcggcggcggcagcggcggcgaggagcccgaggagcccgccgatagcggacaatctctggtgcccgtctacatctacagccccgaatatgtgagcatgtgtgattccctcgccaagatccctaagagagccagcatggtgcattctctgatcgaggcctacgctctgcataagcaaatgaggatcgtgaagcccaaggtcgccagcatggaagagatggccacctttcacaccgatgcctacctccaacatctccagaaggtgtcccaagagggcgacgacgaccaccccgactccattgagtacggactgggctatgattgccccgccaccgagggcatctttgactatgccgccgctatcggcggagctaccatcacagccgcccagtgtctgattgatggcatgtgcaaggtcgccatcaactggtccggaggctggcatcatgccaagaaggatgaggcctccggcttctgttatctgaatgacgccgtgctgggcattctgagactgaggaggaaattcgagaggattctgtacgtggatctggatctgcatcacggagatggagtcgaagatgccttcagcttcaccagcaaggtgatgacagtctctctgcacaagttctcccccggcttctttcccggaaccggcgacgtgtccgacgtgggactgggcaagggaaggtactacagcgtgaacgtgcccattcaagacggcatccaagacgagaagtactaccagatctgcgagtccgtgctcaaggaggtctaccaagccttcaatcctaaggctgtcgtgctccaactgggagctgataccattgctggcgatcccatgtgcagcttcaatatgacacccgtcggaatcggcaagtgcctcaagtacatcctccagtggcagctcgccaccctcattctcggaggaggcggatacaatctggctaataccgccagatgctggacctatctgaccggcgtgattctgggcaaaacactgagcagcgaaatccccgaccacgagtttttcaccgcttacggccccgactacgtgctggagatcacccccagctgcagacccgatagaaacgaaccccatagaatccagcaaattctgaactatatcaagggcaacctcaagcacgtcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 241 - DNA, 2179 bp, synthetic construct
  source 1..2179: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaaaccctataagtgccccgagtgcggcaagagctttagcgatcccggccatctggtgaggcatcagaggacccacaccggcgaaaagccttacaaatgccccgagtgtggaaaaagcttcagcagaagcgatcatctgaccacccatcagaggacacataccggcgagaagccttataaatgccccgaatgtggaaagagcttctccagaagcgaccatctgaccaaccaccagaggacccataccggagaaaaaccttacaaatgccccgagtgtggaaagtccttcagctcccccgccgatctgacaagacatcagagaacccacaccggcgaaaaaccttataaatgtcccgagtgtggcaaaagcttctccgacaagaaggatctgacaagacaccaaaggacccacaccggcgagaaaccttataaatgtcccgaatgcggaaaaagctttagcagaaacgacgctctgaccgaacaccagagaacacataccggagagaaaccctataaatgtcccgagtgcggaaaatccttcagcaccaccggcgctctgacagagcatcagaggacacacaccggcaaaaagacctccgctagcggcagcggcggcggcagcggcggcgaggagcccgaggagcccgccgatagcggacaatctctggtgcccgtctacatctacagccccgaatatgtgagcatgtgtgattccctcgccaagatccctaagagagccagcatggtgcattctctgatcgaggcctacgctctgcataagcaaatgaggatcgtgaagcccaaggtcgccagcatggaagagatggccacctttcacaccgatgcctacctccaacatctccagaaggtgtcccaagagggcgacgacgaccaccccgactccattgagtacggactgggctatgattgccccgccaccgagggcatctttgactatgccgccgctatcggcggagctaccatcacagccgcccagtgtctgattgatggcatgtgcaaggtcgccatcaactggtccggaggctggcatcatgccaagaaggatgaggcctccggcttctgttatctgaatgacgccgtgctgggcattctgagactgaggaggaaattcgagaggattctgtacgtggatctggatctgcatcacggagatggagtcgaagatgccttcagcttcaccagcaaggtgatgacagtctctctgcacaagttctcccccggcttctttcccggaaccggcgacgtgtccgacgtgggactgggcaagggaaggtactacagcgtgaacgtgcccattcaagacggcatccaagacgagaagtactaccagatctgcgagtccgtgctcaaggaggtctaccaagccttcaatcctaaggctgtcgtgctccaactgggagctgataccattgctggcgatcccatgtgcagcttcaatatgacacccgtcggaatcggcaagtgcctcaagtacatcctccagtggcagctcgccaccctcattctcggaggaggcggatacaatctggctaataccgccagatgctggacctatctgaccggcgtgattctgggcaaaacactgagcagcgaaatccccgaccacgagtttttcaccgcttacggccccgactacgtgctggagatcacccccagctgcagacccgatagaaacgaaccccatagaatccagcaaattctgaactatatcaagggcaacctcaagcacgtcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 242 - DNA, 2179 bp, synthetic construct
  source 1..2179: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaagccttacaaatgtcccgaatgcggaaagagcttcagcagagccgacaatctgaccgaacatcagagaacccataccggagaaaaaccttacaaatgtcccgagtgcggcaaaagcttctcccaagccggacatctggccagccaccaaaggacacataccggcgagaaaccctacaagtgccccgagtgcggcaagtccttctctagatccgatgagctggtcagacatcagagaacccataccggcgagaagccttataagtgccccgaatgtggcaagtccttcagccagagagctcatctggagaggcatcaaagaacacacaccggagagaaaccttacaagtgtcccgagtgtggaaagagcttctccagaagggacgagctgaacgtccaccaaagaacccataccggcgaaaagccctataaatgccccgagtgtggaaaatccttttctagatccgaccatctgacaacccaccagaggacccataccggagagaagccctacaaatgccccgagtgtggaaaaagcttctctagaaacgatgctctgacagagcaccaaaggacccacaccggcaaaaagaccagcgctagcggcagcggcggcggcagcggcggcgaggagcccgaggagcccgccgatagcggacaatctctggtgcccgtctacatctacagccccgaatatgtgagcatgtgtgattccctcgccaagatccctaagagagccagcatggtgcattctctgatcgaggcctacgctctgcataagcaaatgaggatcgtgaagcccaaggtcgccagcatggaagagatggccacctttcacaccgatgcctacctccaacatctccagaaggtgtcccaagagggcgacgacgaccaccccgactccattgagtacggactgggctatgattgccccgccaccgagggcatctttgactatgccgccgctatcggcggagctaccatcacagccgcccagtgtctgattgatggcatgtgcaaggtcgccatcaactggtccggaggctggcatcatgccaagaaggatgaggcctccggcttctgttatctgaatgacgccgtgctgggcattctgagactgaggaggaaattcgagaggattctgtacgtggatctggatctgcatcacggagatggagtcgaagatgccttcagcttcaccagcaaggtgatgacagtctctctgcacaagttctcccccggcttctttcccggaaccggcgacgtgtccgacgtgggactgggcaagggaaggtactacagcgtgaacgtgcccattcaagacggcatccaagacgagaagtactaccagatctgcgagtccgtgctcaaggaggtctaccaagccttcaatcctaaggctgtcgtgctccaactgggagctgataccattgctggcgatcccatgtgcagcttcaatatgacacccgtcggaatcggcaagtgcctcaagtacatcctccagtggcagctcgccaccctcattctcggaggaggcggatacaatctggctaataccgccagatgctggacctatctgaccggcgtgattctgggcaaaacactgagcagcgaaatccccgaccacgagtttttcaccgcttacggccccgactacgtgctggagatcacccccagctgcagacccgatagaaacgaaccccatagaatccagcaaattctgaactatatcaagggcaacctcaagcacgtcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 243 - skipped

SEQ ID NO: 244 - DNA, 51 bp, synthetic construct
  source 1..51: mol_type = other DNA
tggatgggagtgtgacagtgctgccttctgaccacaaggtggggcttatac

SEQ ID NO: 245 - DNA, 51 bp, synthetic construct
  source 1..51: mol_type = other DNA
gtataagccccaccttgtggtcagaaggcagcactgtcacactcccatcca

SEQ ID NO: 246 - DNA, 51 bp, synthetic construct
  source 1..51: mol_type = other DNA
attagggaaaccaggaggaggaataccctgtccgtcacggtaccggtaaac

SEQ ID NO: 247 - DNA, 51 bp, synthetic construct
  source 1..51: mol_type = other DNA
caaaagtcaaggtaaacataggcttatggcgccatctagagccgaaaacaa

SEQ ID NO: 248 - DNA, 1343 bp, synthetic construct
  source 1..1343: mol_type = other DNA
aggaaataagagagaaaagaagagtaagaagaaatataagagccaccatggccccaaagaagaagcggaaagtcggaggttcaggcggctcgctggagcccggggagaagccatataagtgtcccgaatgcggaaagtccttcagcgactccggcaaccttagggtccatcagcgaacccacacgggggagaaaccgtacaaatgcccggagtgtgggaagtccttttcccaactggctcacttgcgcgcgcaccagagaactcatactggagaaaagccctacaagtgtccggaatgtggaaagagcttcagcaccaagaactccctcaccgagcaccagcggacccataccggagaaaaaccttacaagtgccctgaatgcggaaagtccttcagcacgtccggttccctggttcggcaccagaggacccacactggagagaagccttacaaatgtccagagtgcggcaaaagcttctcgagccccgccgatttgacccgccaccaaagaactcacaccggggaaaagccgtacaagtgcccggaatgcggcaaatccttctcacaatcctcctctcttgtccggcaccagcgcacacatactggcgaaaaaccatataagtgcccagaatgcgggaagtcgtttagccagtcgggcgatctgaggagacatcagcgcacccacaccggaaagaaaaccagcgcttccggttcgggtggaggatcgggcggagatgccaagtcactgaccgcatggtcccggacccttgtgaccttcaaggacgtgttcgtggacttcaccagagaggagtggaagctgctggacaccgcgcaacagattctctaccggaacgtgatgctggagaactacaagaatctggtgtccctgggctaccagctgaccaagccggacgtgatcctccgcctcgaaaagggggaggagccttggctcgtggagcgggaaatccaccaggagactcacccggactccgagactgccttcgaaatcaagtcatcggtgtccggtggaaagcggcctgcagcgactaagaaggccggacaggccaagaagaagaagggctcatacccgtacgatgtgcccgactacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 249 - DNA, 1343 bp, synthetic construct
  source 1..1343: mol_type = other DNA
aggaaataagagagaaaagaagagtaagaagaaatataagagccaccatggccccaaagaagaagcggaaagtcggaggttcaggcggctcgctggagcccggcgaaaagccctacaagtgtccggagtgcggaaagagcttctcccgctccgataaccttgtgcgccatcagcgaacccacacgggggagaaaccgtacaaatgcccggagtgtgggaagtccttttcccaaaacagcaccctcaccgagcaccagcggacccataccggagaaaaaccttacaagtgccctgaatgcggaaagtccttcagccaatccagctccctggttcggcaccagaggacccacactggagagaagccttacaaatgtccagagtgcggcaaaagcttctcgcaaaagtcgtcgttgattgcccaccaaagaactcacaccggggaaaagccgtacaagtgcccggaatgcggcaaatccttctcaacttccggcaaccttgtccggcaccagcgcacacatactggcgaaaaaccatataagtgcccagaatgcgggaagtcgtttagctcccccgctgatctgaccagacatcagcggacacacaccggagagaaaccctataagtgtcccgaatgtggaaaatccttcagccgctcggacgaactggtccgccatcagcggacacacaccggaaagaaaacttcagcctctggcagcggaggaggttccggtggagatgccaagtcactgaccgcatggtcccggacccttgtgaccttcaaggacgtgttcgtggacttcaccagagaggagtggaagctgctggacaccgcgcaacagattctctaccggaacgtgatgctggagaactacaagaatctggtgtccctgggctaccagctgaccaagccggacgtgatcctccgcctcgaaaagggggaggagccttggctcgtggagcgggaaatccaccaggagactcacccggactccgagactgccttcgaaatcaagtcatcggtgtccggtggaaagcggcctgcagcgactaagaaggccggacaggccaagaagaagaagggctcatacccctacgacgtgccggactacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 250 - DNA, 1343 bp, synthetic construct
  source 1..1343: mol_type = other DNA
aggaaataagagagaaaagaagagtaagaagaaatataagagccaccatggccccaaagaagaagcggaaagtcggaggttcaggcggctcgctggaaccgggagagaagccatacaaatgccccgagtgtggaaagtccttttcgacttccggttcgcttgtgcgccatcagcgaacccacacgggggagaaaccgtacaaatgcccggagtgtgggaagtccttttcccaacgggccaacctgagagcccaccaaaggacgcataccggcgaaaagccctacaagtgtcccgaatgtggaaagagcttctctcgcactgacaccctccgggatcaccagcggacccataccggagaaaaaccttacaagtgccctgaatgcggaaagtccttcagccggagggatgaactgaacgtccaccagaggacccacactggagagaagccttacaaatgtccagagtgcggcaaaagcttctcgcatcgcaccaccttgaccaaccaccaaagaactcacaccggggaaaagccgtacaagtgcccggaatgcggcaaatccttctcacgctccgacaagcttactgaacaccagcgcacacatactggcgaaaaaccatataagtgcccagaatgcgggaagtcgtttagccagaagtccagcctgattgctcaccagcgcacccacaccggaaagaaaacctccgcatccggaagcggtggaggaagtggcggggatgccaagtcactgaccgcatggtcccggacccttgtgaccttcaaggacgtgttcgtggacttcaccagagaggagtggaagctgctggacaccgcgcaacagattctctaccggaacgtgatgctggagaactacaagaatctggtgtccctgggctaccagctgaccaagccggacgtgatcctccgcctcgaaaagggggaggagccttggctcgtggagcgggaaatccaccaggagactcacccggactccgagactgccttcgaaatcaagtcatcggtgtccggtggaaagcggcctgcagcgactaagaaggccggacaggccaagaagaagaagggctcatacccgtacgatgtgcccgactatgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 251 - DNA, 1343 bp, synthetic construct
  source 1..1343: mol_type = other DNA
aggaaataagagagaaaagaagagtaagaagaaatataagagccaccatggccccaaagaagaagcggaaagtcggaggttcaggcggctcgctggaacctggggagaagccatataagtgccctgagtgtggaaagtccttctcccgggctgacaatctgaccgaacaccagcggactcacactggagagaagccgtacaaatgccccgagtgcggaaagtcgttctcccgctccgaccatctcaccacccatcagagaacccacaccggggaaaagccatacaagtgtcctgaatgcggcaaatcattcagccgctcggacaacctcgtgcggcatcaacgaacccacacgggagagaaaccgtataaatgcccggagtgcggcaaaagctttagccagaactccactctgacggagcaccaacgcacccataccggagaaaagccttacaagtgcccagagtgcggtaaaagcttcagccagtcctcctcactcgtgcgccaccagagaactcatactggcgaaaagccgtataagtgcccggaatgtggaaagagcttcagtcagaagtccagcctgattgcgcaccagaggactcataccggcgaaaaaccctacaaatgtccggaatgcggaaaatcgttctcaacttccggcaaccttgtgcggcaccagcgcacacacaccggcaaaaagacttcggcatccggttcgggaggaggatcaggaggagatgccaagtcactgaccgcatggtcccggacccttgtgaccttcaaggacgtgttcgtggacttcaccagagaggagtggaagctgctggacaccgcgcaacagattctctaccggaacgtgatgctggagaactacaagaatctggtgtccctgggctaccagctgaccaagccggacgtgatcctccgcctcgaaaagggggaggagccttggctcgtggagcgggaaatccaccaggagactcacccggactccgagactgccttcgaaatcaagtcatcggtgtccggtggaaagcggcctgcagcgactaagaaggccggacaggccaagaagaagaagggctcatacccttacgatgtgccggattacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 252 - DNA, 1343 bp, synthetic construct
  source 1..1343: mol_type = other DNA
aggaaataagagagaaaagaagagtaagaagaaatataagagccaccatggccccaaagaagaagcggaaagtcggaggttcaggcggctcgctcgaacccggagaaaagccctacaaatgcccagagtgcggaaagagcttttcccgctccgatcatctcaccacgcaccagcggacccataccggagaaaaaccttacaagtgccctgaatgcggaaagtccttcagcgaccccggtcacctggttcggcaccagaggacccacactggagagaagccttacaaatgtccagagtgcggcaaaagcttctcgcacaagaacgccttgcaaaaccaccaacgcactcataccggcgaaaagccatacaagtgtccggagtgtgggaagtccttctcgcgacgcgacgaactgaacgtccaccaaagaactcacaccggggaaaagccgtacaagtgcccggaatgcggcaaatccttctcacaacgcgccaaccttagggctcaccagcgcacacatactggcgaaaaaccatataagtgcccagaatgcgggaagtcgtttagcacgactggcaacctgaccgtccatcagcggacacacaccggagagaaaccctataagtgtcccgaatgtggaaaatccttcagctctaagaagcatctggccgagcaccagagaacccacactgggaagaaaaccagcgcatcaggaagcggaggaggttccggtggcgatgccaagtcactgaccgcatggtcccggacccttgtgaccttcaaggacgtgttcgtggacttcaccagagaggagtggaagctgctggacaccgcgcaacagattctctaccggaacgtgatgctggagaactacaagaatctggtgtccctgggctaccagctgaccaagccggacgtgatcctccgcctcgaaaagggggaggagccttggctcgtggagcgggaaatccaccaggagactcacccggactccgagactgccttcgaaatcaagtcatcggtgtccggtggaaagcggcctgcagcgactaagaaggccggacaggccaagaagaagaagggctcatacccttacgacgtcccggattacgcgtaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 253 - DNA, 1343 bp, synthetic construct
  source 1..1343: mol_type = other DNA
aggaaataagagagaaaagaagagtaagaagaaatataagagccaccatggccccaaagaagaagcggaaagtcggaggttcaggcggctcgctggagcctggagagaagccgtataagtgcccagaatgtggaaagtcattcagctccaagaaggctctgaccgaacaccaacggacgcacaccggagaaaaaccctacaaatgccctgaatgcgggaagtccttttccacctccggcaacctgaccgagcaccagcggactcacactggagagaaaccttacaagtgccctgagtgtggaaagagcttctcccatcgcactacccttaccaatcaccaacgcacccataccggcgaaaaaccatacaaatgtccagagtgcggcaaatccttcagccgcaacgatgcgctcaccgagcatcagcgcactcatactggcgaaaagccctataagtgtccggaatgcggaaagtcgttcagcgactccggaaaccttagagtgcaccagaggacacacactggggaaaagccgtacaagtgtcccgaatgcggcaaaagcttcagccagtccggggatctgcggaggcaccagcgaacccatacgggagaaaagccttataaatgcccggagtgcggaaagagtttctcgcggagcgaccacctgaccaaccaccagagaacccacactggcaaaaagacctcggcatccggttcaggaggagggtccggaggagatgccaagtcactgaccgcatggtcccggacccttgtgaccttcaaggacgtgttcgtggacttcaccagagaggagtggaagctgctggacaccgcgcaacagattctctaccggaacgtgatgctggagaactacaagaatctggtgtccctgggctaccagctgaccaagccggacgtgatcctccgcctcgaaaagggggaggagccttggctcgtggagcgggaaatccaccaggagactcacccggactccgagactgccttcgaaatcaagtcatcggtgtccggtggaaagcggcctgcagcgactaagaaggccggacaggccaagaagaagaagggctcatacccgtacgacgtgcccgattacgcttaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 254 - DNA, 21 bp, synthetic construct
  source 1..21: mol_type = other DNA
gcagtaacagttcctagaaac

SEQ ID NO: 255 - DNA, 21 bp, synthetic construct
  source 1..21: mol_type = other DNA
gtgacagatatagtactagag

SEQ ID NO: 256 - DNA, 21 bp, synthetic construct
  source 1..21: mol_type = other DNA
atacggagtatgacgaaagtt

SEQ ID NO: 257 - DNA, 21 bp, synthetic construct
  source 1..21: mol_type = other DNA
gatatagtactagagtggcag

SEQ ID NO: 258 - DNA, 21 bp, synthetic construct
  source 1..21: mol_type = other DNA
cccaataaaatgattggctgg

SEQ ID NO: 259 - DNA, 21 bp, synthetic construct
  source 1..21: mol_type = other DNA
agggcaaacctgagtcatcac

SEQ ID NO: 260 - protein, 949 aa, synthetic construct
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 261 - protein, 949 aa, synthetic construct
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 262 - protein, 949 aa, synthetic construct
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 263 - protein, 949 aa, synthetic construct
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 264 - protein, 949 aa, synthetic construct
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 265 - protein, 949 aa, synthetic construct
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 266 - protein, 949 aa, synthetic construct
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 267 - protein, 949 aa, synthetic construct
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 268 - protein, 798 aa, synthetic construct
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRV

SEQ ID NO: 269 - protein, 798 aa, synthetic construct
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRV

SEQ ID NO: 270 - protein, 798 aa, synthetic construct
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRV

SEQ ID NO: 271 - protein, 798 aa, synthetic construct
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRV

SEQ ID NO: 272 - protein, 798 aa, synthetic construct
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRV

SEQ ID NO: 273 - protein, 798 aa, synthetic construct
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRV

SEQ ID NO: 274 - protein, 798 aa, synthetic construct
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRV

SEQ ID NO: 275 - protein, 798 aa, synthetic construct
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRV

SEQ ID NO: 276 - DNA, 2394 bp, synthetic construct
  source 1..2394: mol_type = other DNA
catatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacggtggcggcaagcaggctctggaaaccgtccagcgcctccttcctgtcctgtgtcaagcgcacgggctcacacctgaacaagtagtggccatcgcttccaacattggtggcaagcaggccctggaaactgtacagcggttgttgcctgtcctctgccaagcgcatgggcttaccccggagcaagtggtcgctatcgcttctcatgatggtgggaagcaggcgctggaaacagtgcagagactgcttccggtgctgtgccaggcacatggcctgacgccggaacaagtggtggcgattgcatctaacggaggcgggaagcaggcgctggagacagtgcaacggctgctccctgttctatgccaagctcatggactcacccccgagcaagtcgtcgccattgcgtccaacaatggaggcaaacaggcactggaaacggtgcaaagactgttgcctgtactgtgccaggctcatggccttactcccgaacaggtggtggcaattgcgagtaacatcggcggaaagcaggctttggaaactgtgcagcgcttgctgcctgtgctctgtcaggctcacggactcacccccgagcaagtcgtagccatcgcctcaaatatcggaggcaagcaagcgctcgagactgtgcagaggctcctgcctgtgttatgtcaggcacacgggctgacccctgaacaagtcgtggccattgcctccaacaacggcggaaaacaggcgctcgaaacagtccagaggcttttgcccgtgctgtgtcaagctcacggcctgactccggagcaagtggtcgccattgcatcccatgatggcggtaaacaagcattggaaacggtccaacgcctgctgccggtcctgtgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcacacgacggagggaagcaagcccttgaaaccgtgcaaaggctcctcccggtgctgtgccaagcccatggattgactccagaacaggtggtcgctatcgcatcacatgacggtggaaagcaagccctcgaaactgtgcagcggctcctccctgtgttgtgccaggcgcacggtctgactcccgagcaagtcgtggcaatcgcctcgaacatcgggggcaaacaagctctcgaaactgttcaaagactgctccccgtgctctgtcaagctcacggcctcactcctgaacaggtagtcgcgattgcctcccatgacgggggaaagcaggccttggagacagtccagcggctcttgccggtcttgtgccaagcacacggattgactccagaacaggtggtcgcaatcgccagcaatatcggggggaagcaagccttggaaaccgttcagcggctactccctgtgctgtgtcaagcccacggtctgaccccagaacaagtggtggctatcgcgtcgaacattggaggcaaacaggccctcgagactgtccagagactcctgccagtcctctgccaggctcatgggctgactcctgagcaagttgtggccatagcatccaacggcggaggaaagcaagctctggaaaccgtacagagacttctcccggtgttgtgtcaggcccacggacttactccggaacaggttgtcgccatcgcgtccaacggtggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtc

SEQ ID NO: 277 - DNA, 2394 bp, synthetic construct
  source 1..2394: mol_type = other DNA
catatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacattggcggcaagcaggctctggaaaccgtccagcgcctccttcctgtcctgtgtcaagcgcacgggctcacacctgaacaagtagtggccatcgcttcccatgatggtggcaagcaggccctggaaactgtacagcggttgttgcctgtcctctgccaagcgcatgggcttaccccggagcaagtggtcgctatcgcttctaacggcggtgggaagcaggcgctggaaacagtgcagagactgcttccggtgctgtgccaggcacatggcctgacgccggaacaagtggtggcgattgcatctcacgatggcgggaagcaggcgctggagacagtgcaacggctgctccctgttctatgccaagctcatggactcacccccgagcaagtcgtcgccattgcgtcccacgatggaggcaaacaggcactggaaacggtgcaaagactgttgcctgtactgtgccaggctcatggccttactcccgaacaggtggtggcaattgcgagtaacaacggcggaaagcaggctttggaaactgtgcagcgcttgctgcctgtgctctgtcaggctcacggactcacccccgagcaagtcgtagccatcgcctcaaatggaggaggcaagcaagcgctcgagactgtgcagaggctcctgcctgtgttatgtcaggcacacgggctgacccctgaacaagtcgtggccattgcctccaacatcggcggaaaacaggcgctcgaaacagtccagaggcttttgcccgtgctgtgtcaagctcacggcctgactccggagcaagtggtcgccattgcatccaacggaggcggtaaacaagcattggaaacggtccaacgcctgctgccggtcctgtgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcaaacggcggagggaagcaagcccttgaaaccgtgcaaaggctcctcccggtgctgtgccaagcccatggattgactccagaacaggtggtcgctatcgcatcaaacaatggtggaaagcaagccctcgaaactgtgcagcggctcctccctgtgttgtgccaggcgcacggtctgactcccgagcaagtcgtggcaatcgcctcgaacatcggtgggaagcaagctctggagactgtgcagaggctcctgccggtgctttgtcaggcccacgggcttactccggaacaagtggtggccatcgcctccaacggggggggcaaacaggccttggagacagtccagcggctcttgccggtcttgtgccaagcacacggattgactccagaacaggtggtcgcaatcgccagcaatatcggggggaagcaagccttggaaaccgttcagcggctactccctgtgctgtgtcaagcccacggtctgaccccagaacaagtggtggctatcgcgtcgaacattggaggcaaacaggccctcgagactgtccagagactcctgccagtcctctgccaggctcatgggctgactcctgagcaagttgtggccatagcatccaacaacggaggaaagcaagctctggaaaccgtacagagacttctcccggtgttgtgtcaggcccacggacttactccggaacaggttgtcgccatcgcgtccaacaacggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtc

SEQ ID NO: 278 - DNA, 2394 bp, synthetic construct
  source 1..2394: mol_type = other DNA
catatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacaacggcggcaagcaggctctggaaaccgtccagcgcctccttcctgtcctgtgtcaagcgcacgggctcacacctgaacaagtagtggccatcgcttccaacattggtggcaagcaggccctggaaactgtacagcggttgttgcctgtcctctgccaagcgcatgggcttaccccggagcaagtggtcgctatcgcttctaacaacggtgggaagcaggcgctggaaacagtgcagagactgcttccggtgctgtgccaggcacatggcctgacgccggaacaagtggtggcgattgcatctaacggaggcgggaagcaggcgctggagacagtgcaacggctgctccctgttctatgccaagctcatggactcacccccgagcaagtcgtcgccattgcgtcccacgatggcggaaagcaggctttggaaactgtgcagcgcttgctgcctgtgctctgtcaggctcacggactcacccccgagcaagtcgtagccatcgcctcaaatatcggaggcaagcaagcgctcgagactgtgcagaggctcctgcctgtgttatgtcaggcacacgggctgacccctgaacaagtcgtggccattgcctccaacggtggcggaaaacaggcgctcgaaacagtccagaggcttttgcccgtgctgtgtcaagctcacggcctgactccggagcaagtggtcgccattgcatcccatgatggcggtaaacaagcattggaaacggtccaacgcctgctgccggtcctgtgccaggcccatggtctgactcccgaacaggtcgtggctatcgcctccaacatcggtggaaaacaagccctggagactgtacagaggctgctgcccgtgctatgtcaagcacatggacttacgcctgaacaagtggtggcaattgcatcacacgacggagggaagcaagcccttgaaaccgtgcaacgacttctgcccgtcctgtgtcaggcacacggtctgactcccgaacaggtggtggccatcgccagcaatatcgggggcaagcaggcgttggaaactgtgcaaaggctcctcccggtgctgtgccaagcccatggattgactccagaacaggtggtcgctatcgcatcacatgacggtggaaagcaagccctcgaaactgtgcagcggctcctccctgtgttgtgccaggcgcacggtctgactcccgagcaagtcgtggcaatcgcctcgaacggagggggcaaacaagctctcgaaactgttcaaagactgctccccgtgctctgtcaagctcacggcctcactcctgaacaggtagtcgcgattgcctccaacggggggggaaagcaggccttggagacagtccagcggctcttgccggtcttgtgccaagcacacggattgactccagaacaggtggtcgcaatcgccagccacgacggggggaagcaagccttggaaaccgttcagcggctactccctgtgctgtgtcaagcccacggtctgaccccagaacaagtggtggctatcgcgtcgcacgatggaggcaaacaggccctcgagactgtccagagactcctgccagtcctctgccaggctcatgggctgactcctgagcaagttgtggccatagcatccaacggcggaggaaagcaagctctggaaaccgtacagagacttctcccggtgttgtgtcaggcccacggacttactccggaacaggttgtcgccatcgcgtccaacataggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtc

SEQ ID NO: 279 - DNA, 2394 bp, synthetic construct
  source 1..2394: mol_type = other DNA
catatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacaacggcggcaagcaggctctggaaaccgtccagcgcctccttcctgtcctgtgtcaagcgcacgggctcacacctgaacaagtagtggccatcgcttccaacattggtggcaagcaggccctggaaactgtacagcggttgttgcctgtcctctgccaagcgcatgggcttaccccggagcaagtggtcgctatcgcttctcatgatggtgggaagcaggcgctggaaacagtgcagagactgcttccggtgctgtgccaggcacatggcctgacgccggaacaagtggtggcgattgcatctaacggaggcgggaagcaggcgctggagacagtgcaacggctgctccctgttctatgccaagctcatggactcacccccgagcaagtcgtcgccattgcgtcccacgacggaggcaaacaggcactggaaacggtgcaaagactgttgcctgtactgtgccaggctcatggccttactcccgaacaggtggtggcaattgcgagtaacatcggcggaaagcaggctttggaaactgtgcagcgcttgctgcctgtgctctgtcaggctcacggactcacccccgagcaagtcgtagccatcgcctcaaataacggaggcaagcaagcgctcgagactgtgcagaggctcctgcctgtgttatgtcaggcacacgggctgacccctgaacaagtcgtggccattgcctccaacaacggcggaaaacaggcgctcgaaacagtccagaggcttttgcccgtgctgtgtcaagctcacggcctgactccggagcaagtggtcgccattgcatccaatggcggcggtaaacaagcattggaaacggtccaacgcctgctgccggtcctgtgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcaaacgggggagggaagcaagcccttgaaaccgtgcaaaggctcctcccggtgctgtgccaagcccatggattgactccagaacaggtggtcgctatcgcatcaaacaacggtggaaagcaagccctcgaaactgtgcagcggctcctccctgtgttgtgccaggcgcacggtctgactcccgagcaagtcgtggcaatcgcctcgcacgatgggggcaaacaagctctcgaaactgttcaaagactgctccccgtgctctgtcaagctcacggcctcactcctgaacaggtagtcgcgattgcctcccatgacgggggaaagcaggccttggagacagtccagcggctcttgccggtcttgtgccaagcacacggattgactccagaacaggtggtcgcaatcgccagccatgatggggggaagcaagccttggaaaccgttcagcggctactccctgtgctgtgtcaagcccacggtctgaccccagaacaagtggtggctatcgcgtcgaacggaggaggcaaacaggccctcgagactgtccagagactcctgccagtcctctgccaggctcatgggctgactcctgagcaagttgtggccatagcatccaacaatggaggaaagcaagctctggaaaccgtacagagacttctcccggtgttgtgtcaggcccacggacttactccggaacaggttgtcgccatcgcgtccaacatcggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtc

SEQ ID NO: 280 - DNA, 2394 bp, synthetic construct
  source 1..2394: mol_type = other DNA
catatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctcaccccggaacaggtggtggcgattgcttcccacgacggtggcaagcaggccttggaaaccgtccaacggcttttgccggtgctttgccaggcccatggactgacccccgaacaagtcgttgccattgctagtcacgacggcggcaagcaggctctggaaaccgtccagcgcctccttcctgtcctgtgtcaagcgcacgggctcacacctgaacaagtagtggccatcgcttccaacggcggtggcaagcaggccctggaaactgtacagcggttgttgcctgtcctctgccaagcgcatgggcttaccccggagcaagtggtcgctatcgcttctaataatggtgggaagcaggcgctggaaacagtgcagagactgcttccggtgctgtgccaggcacatggcctgacgccggaacaagtggtggcgattgcatctaacattggcgggaagcaggcgctggagacagtgcaacggctgctccctgttctatgccaagctcatggactcacccccgagcaagtcgtcgccattgcgtccaacaacggaggcaaacaggcactggaaacggtgcaaagactgttgcctgtactgtgccaggctcatggccttactcccgaacaggtggtggcaattgcgagtaacaacggtggaaagcaggcgctcgaaactgtccagagattgctgcctgtgctgtgtcaggcacacggactgaccccagaacaagtggtggccatcgcgagccatgacggcggaaagcaggctttggaaactgtgcagcgcttgctgcctgtgctctgtcaggctcacggactcacccccgagcaagtcgtagccatcgcctcaaatatcggaggcaagcaagcgctcgagactgtgcagaggctcctgcctgtgttatgtcaggcacacgggctgacccctgaacaagtcgtggccattgcctccaacggtggcggaaaacaggcgctcgaaacagtccagaggcttttgcccgtgctgtgtcaagctcacggcctgactccggagcaagtggtcgccattgcatccaataacggcggtaaacaagcattggaaacggtccaacgcctgctgccggtcctgtgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcacacgacggagggaagcaagcccttgaaaccgtgcaaaggctcctcccggtgctgtgccaagcccatggattgactccagaacaggtggtcgctatcgcatcaaacatcggtggaaagcaagccctcgaaactgtgcagcggctcctccctgtgttgtgccaggcgcacggtctgactcccgagcaagtcgtggcaatcgcctcgaacaatgggggcaaacaagctctcgaaactgttcaaagactgctccccgtgctctgtcaagctcacggcctcactcctgaacaggtagtcgcgattgcctcccatgacgggggaaagcaggccttggagacagtccagcggctcttgccggtcttgtgccaagcacacggattgactccagaacaggtggtcgcaatcgccagccatgatggaggaaagcaagctctggaaaccgtacagagacttctcccggtgttgtgtcaggcccacggacttactccggaacaggttgtcgccatcgcgtccaacatcggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtc

SEQ ID NO: 281 - DNA, 2394 bp, synthetic construct
  source 1..2394: mol_type = other DNA
catatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtcatgacggcggcaagcaggctctggaaaccgtccagcgcctccttcctgtcctgtgtcaagcgcacgggctcacacctgaacaagtagtggccatcgcttccaacggaggtggcaagcaggccctggaaactgtacagcggttgttgcctgtcctctgccaagcgcatgggcttaccccggagcaagtggtcgctatcgcttctaacaacggaggcaagcaagcgctcgagactgtgcagaggctcctgcctgtgttatgtcaggcacacgggctgacccctgaacaagtcgtggccattgcctccaacatcggcggaaaacaggcgctcgaaacagtccagaggcttttgcccgtgctgtgtcaagctcacggcctgactccggagcaagtggtcgccattgcatccaacaacggcggtaaacaagcattggaaacggtccaacgcctgctgccggtcctgtgccaggcccatggtctgactcccgaacaggtcgtggctatcgcctccaacggaggtggtaagcaagcactggaaaccgtgcagcggttgctccccgtgctctgtcaggctcacgggctcactccagagcaggtggtggccattgccagccatgacggtggaaaacaagccctggagactgtacagaggctgctgcccgtgctatgtcaagcacatggacttacgcctgaacaagtggtggcaattgcatcaaacattggagggaagcaagcccttgaaaccgtgcaacgacttctgcccgtcctgtgtcaggcacacggtctgactcccgaacaggtggtggccatcgccagccacgacgggggcaagcaggcgttggaaactgtgcaaaggctcctcccggtgctgtgccaagcccatggattgactccagaacaggtggtcgctatcgcatcacatgacggtggaaagcaagccctcgaaactgtgcagcggctcctccctgtgttgtgccaggcgcacggtctgactcccgagcaagtcgtggcaatcgcctcgaacggagggggcaaacaagctctcgaaactgttcaaagactcttgccggtcttgtgccaagcacacggattgactccagaacaggtggtcgcaatcgccagccacgacggcgggaagcaggcactcgaaactgtccagcgtctgctgcctgtcctctgccaagcccacggcctcactccagaacaggtggtcgcgattgcaagcaacggcggggggaagcaagccttggaaaccgttcagcggctactccctgtgctgtgtcaagcccacggtctgaccccagaacaagtggtggctatcgcgtcgcacgatggaggcaaacaggccctcgagactgtccagagactcctgccagtcctctgccaggctcatgggctgactcctgagcaagttgtggccatagcatccaacggcggcggcaagcaagccctggagactgtgcaacggctgttgccggtcctgtgccaggcccatggtcttacgccggaacaggtcgtggccatcgcgtcaaacaacggtggcaagcaggctctggagacagtgcaacgccttctgcctgtgctttgccaggcccacggcttgacccccgagcaagtggtggccatcgcctcgaataacggaggaaagcaagctctggaaaccgtacagagacttctcccggtgttgtgtcaggcccacggacttactccggaacaggttgtcgccatcgcgtccaacataggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtc

SEQ ID NO: 282 - DNA, 2394 bp, synthetic construct
  source 1..2394: mol_type = other DNA
catatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtcatgacggcggcaagcaggctctggaaaccgtccagcgcctccttcctgtcctgtgtcaagcgcacgggctcacacctgaacaagtagtggccatcgcttcccatgatggtggcaagcaggccctggaaactgtacagcggttgttgcctgtcctctgccaagcgcatgggcttaccccggagcaagtggtcgctatcgcttctaacggcggcggcaaacaggcactggaaacggtgcaaagactgttgcctgtactgtgccaggctcatggccttactcccgaacaggtggtggcaattgcgagtaacaacggcggaaagcaggctttggaaactgtgcagcgcttgctgcctgtgctctgtcaggctcacggactcacccccgagcaagtcgtagccatcgcctcaaatggaggaggcaagcaagcgctcgagactgtgcagaggctcctgcctgtgttatgtcaggcacacgggctgacccctgaacaagtcgtggccattgcctccaacaacggcggaaaacaggcgctcgaaacagtccagaggcttttgcccgtgctgtgtcaagctcacggcctgactccggagcaagtggtcgccattgcatcccacgacggcggtaaacaagcattggaaacggtccaacgcctgctgccggtcctgtgccaggcccatgggctgactccggaacaagtcgtcgcgattgcgtccaacatcgggggaaagcaagcactcgaaacagtgcagcggctgctgccagtcctgtgccaggcacatggcctgaccccggagcaggtggtggccatcgcgagcaacattggtgggaagcaggcactggaaactgtccaacggctgctgccggtgctatgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcaaacaacggagggaagcaagcccttgaaaccgtgcaaaggctcctcccggtgctgtgccaagcccatggattgactccagaacaggtggtcgctatcgcatcaaacggaggtggaaagcaagccctcgaaactgtgcagcggctcctccctgtgttgtgccaggcgcacggtctgactcccgagcaagtcgtggcaatcgcctcgcacgacggtgggaagcaagctctggagactgtgcagaggctcctgccggtgctttgtcaggcccacgggcttactccggaacaagtggtggccatcgcctccaacggggggggcaaacaggccttggagacagtccagcggctcttgccggtcttgtgccaagcacacggattgactccagaacaggtggtcgcaatcgccagcaatggcggggggaagcaagccttggaaaccgttcagcggctactccctgtgctgtgtcaagcccacggtctgaccccagaacaagtggtggctatcgcgtcgaacgggggaggcaaacaggccctcgagactgtccagagactcctgccagtcctctgccaggctcatgggctgactcctgagcaagttgtggccatagcatccaacaacggaggaaagcaagctctggaaaccgtacagagacttctcccggtgttgtgtcaggcccacggacttactccggaacaggttgtcgccatcgcgtccaacaacggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtc

SEQ ID NO: 283 - DNA, 2394 bp, synthetic construct
  source 1..2394: mol_type = other DNA
catatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacggtggcggcaagcaggctctggaaaccgtccagcgcctccttcctgtcctgtgtcaagcgcacgggctcacacctgaacaagtagtggccatcgcttccaacaatggtggcaagcaggccctggaaactgtacagcggttgttgcctgtcctctgccaagcgcatgggcttaccccggagcaagtggtcgctatcgcttctcatgatggtgggaagcaggcgctggaaacagtgcagagactgcttccggtgctgtgccaggcacatggcctgacgccggaacaagtggtggcgattgcatctcacgatggcgggaagcaggcgctggagacagtgcaacggctgctccctgttctatgccaagctcatggactcacccccgagcaagtcgtcgccattgcgtccaacggaggaggcaaacaggcactggaaacggtgcaaagactgttgcctgtactgtgccaggctcatggccttactcccgaacaggtggtggcaattgcgagtcacgacggcggaaagcaggctttggaaactgtgcagcgcttgctgcctgtgctctgtcaggctcacggactcacccccgagcaagtcgtagccatcgcctcaaatatcggaggcaagcaagcgctcgagactgtgcagaggctcctgcctgtgttatgtcaggcacacgggctgacccctgaacaagtcgtggccattgcctccaacaacggcggaaaacaggcgctcgaaacagtccagaggcttttgcccgtgctgtgtcaagctcacggcctgactccggagcaagtggtcgccattgcatccaacaatggcggtaaacaagcattggaaacggtccaacgcctgctgccggtcctgtgccaggcccatggtctgactcccgaacaggtcgtggctatcgcctccaacatcggagggaagcaagcccttgaaaccgtgcaaaggctcctcccggtgctgtgccaagcccatggattgactccagaacaggtggtcgctatcgcatcaaacaacggtggaaagcaagccctcgaaactgtgcagcggctcctccctgtgttgtgccaggcgcacggtctgactcccgagcaagtcgtggcaatcgcctcgaacggagggggcaaacaagctctcgaaactgttcaaagactgctccccgtgctctgtcaagctcacggcctcactcctgaacaggtagtcgcgattgcctcccatgacgggggaaagcaggccttggagacagtccagcggctcttgccggtcttgtgccaagcacacggattgactccagaacaggtggtcgcaatcgccagcaatatcggcggcaagcaagccctggagactgttcagaggctcttgcctgtgctctgccaggcccatggcttgacacctgaacaggtggtggccatcgcgagcaacggtggggggaagcaagccttggaaaccgttcagcggctactccctgtgctgtgtcaagcccacggtctgaccccagaacaagtggtggctatcgcgtcgaacaacggaggcaaacaggccctcgagactgtccagagactcctgccagtcctctgccaggctcatgggctgactcctgagcaagttgtggccatagcatccaacaacggaggaaagcaagctctggaaaccgtacagagacttctcccggtgttgtgtcaggcccacggacttactccggaacaggttgtcgccatcgcgtccaacaacggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtc

SEQ ID NO: 284 - DNA, 3125 bp, synthetic construct
  source 1..3125: mol_type = other DNA
aggaaataagagagaaaagaagagtaagaagaaatataagagccaccatggccccaaagaagaagcggaaagtcggaggttcaggcggctcgcatatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacggtggcggcaagcaggctctggaaaccgtccagcgcctccttcctgtcctgtgtcaagcgcacgggctcacacctgaacaagtagtggccatcgcttccaacattggtggcaagcaggccctggaaactgtacagcggttgttgcctgtcctctgccaagcgcatgggcttaccccggagcaagtggtcgctatcgcttctcatgatggtgggaagcaggcgctggaaacagtgcagagactgcttccggtgctgtgccaggcacatggcctgacgccggaacaagtggtggcgattgcatctaacggaggcgggaagcaggcgctggagacagtgcaacggctgctccctgttctatgccaagctcatggactcacccccgagcaagtcgtcgccattgcgtccaacaatggaggcaaacaggcactggaaacggtgcaaagactgttgcctgtactgtgccaggctcatggccttactcccgaacaggtggtggcaattgcgagtaacatcggcggaaagcaggctttggaaactgtgcagcgcttgctgcctgtgctctgtcaggctcacggactcacccccgagcaagtcgtagccatcgcctcaaatatcggaggcaagcaagcgctcgagactgtgcagaggctcctgcctgtgttatgtcaggcacacgggctgacccctgaacaagtcgtggccattgcctccaacaacggcggaaaacaggcgctcgaaacagtccagaggcttttgcccgtgctgtgtcaagctcacggcctgactccggagcaagtggtcgccattgcatcccatgatggcggtaaacaagcattggaaacggtccaacgcctgctgccggtcctgtgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcacacgacggagggaagcaagcccttgaaaccgtgcaaaggctcctcccggtgctgtgccaagcccatggattgactccagaacaggtggtcgctatcgcatcacatgacggtggaaagcaagccctcgaaactgtgcagcggctcctccctgtgttgtgccaggcgcacggtctgactcccgagcaagtcgtggcaatcgcctcgaacatcgggggcaaacaagctctcgaaactgttcaaagactgctccccgtgctctgtcaagctcacggcctcactcctgaacaggtagtcgcgattgcctcccatgacgggggaaagcaggccttggagacagtccagcggctcttgccggtcttgtgccaagcacacggattgactccagaacaggtggtcgcaatcgccagcaatatcggggggaagcaagccttggaaaccgttcagcggctactccctgtgctgtgtcaagcccacggtctgaccccagaacaagtggtggctatcgcgtcgaacattggaggcaaacaggccctcgagactgtccagagactcctgccagtcctctgccaggctcatgggctgactcctgagcaagttgtggccatagcatccaacggcggaggaaagcaagctctggaaaccgtacagagacttctcccggtgttgtgtcaggcccacggacttactccggaacaggttgtcgccatcgcgtccaacggtggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtcgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagaggcctgccgctaccaagaaagcaggacaagccaagaagaagaagggatcctacccgtatgacgtgcccgactacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 285 - DNA, 3125 bp, synthetic construct
  source 1..3125: mol_type = other DNA
aggaaataagagagaaaagaagagtaagaagaaatataagagccaccatggccccaaagaagaagcggaaagtcggaggttcaggcggctcgcatatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacattggcggcaagcaggctctggaaaccgtccagcgcctccttcctgtcctgtgtcaagcgcacgggctcacacctgaacaagtagtggccatcgcttcccatgatggtggcaagcaggccctggaaactgtacagcggttgttgcctgtcctctgccaagcgcatgggcttaccccggagcaagtggtcgctatcgcttctaacggcggtgggaagcaggcgctggaaacagtgcagagactgcttccggtgctgtgccaggcacatggcctgacgccggaacaagtggtggcgattgcatctcacgatggcgggaagcaggcgctggagacagtgcaacggctgctccctgttctatgccaagctcatggactcacccccgagcaagtcgtcgccattgcgtcccacgatggaggcaaacaggcactggaaacggtgcaaagactgttgcctgtactgtgccaggctcatggccttactcccgaacaggtggtggcaattgcgagtaacaacggcggaaagcaggctttggaaactgtgcagcgcttgctgcctgtgctctgtcaggctcacggactcacccccgagcaagtcgtagccatcgcctcaaatggaggaggcaagcaagcgctcgagactgtgcagaggctcctgcctgtgttatgtcaggcacacgggctgacccctgaacaagtcgtggccattgcctccaacatcggcggaaaacaggcgctcgaaacagtccagaggcttttgcccgtgctgtgtcaagctcacggcctgactccggagcaagtggtcgccattgcatccaacggaggcggtaaacaagcattggaaacggtccaacgcctgctgccggtcctgtgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcaaacggcggagggaagcaagcccttgaaaccgtgcaaaggctcctcccggtgctgtgccaagcccatggattgactccagaacaggtggtcgctatcgcatcaaacaatggtggaaagcaagccctcgaaactgtgcagcggctcctccctgtgttgtgccaggcgcacggtctgactcccgagcaagtcgtggcaatcgcctcgaacatcggtgggaagcaagctctggagactgtgcagaggctcctgccggtgctttgtcaggcccacgggcttactccggaacaagtggtggccatcgcctccaacggggggggcaaacaggccttggagacagtccagcggctcttgccggtcttgtgccaagcacacggattgactccagaacaggtggtcgcaatcgccagcaatatcggggggaagcaagccttggaaaccgttcagcggctactccctgtgctgtgtcaagcccacggtctgaccccagaacaagtggtggctatcgcgtcgaacattggaggcaaacaggccctcgagactgtccagagactcctgccagtcctctgccaggctcatgggctgactcctgagcaagttgtggccatagcatccaacaacggaggaaagcaagctctggaaaccgtacagagacttctcccggtgttgtgtcaggcccacggacttactccggaacaggttgtcgccatcgcgtccaacaacggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtcgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagaggcctgccgctaccaagaaagcaggacaagccaagaagaagaagggatcctacccgtatgacgtgcccgactacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 286 - DNA, 3125 bp, synthetic construct
  source 1..3125: mol_type = other DNA
aggaaataagagagaaaagaagagtaagaagaaatataagagccaccatggccccaaagaagaagcggaaagtcggaggttcaggcggctcgcatatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacaacggcggcaagcaggctctggaaaccgtccagcgcctccttcctgtcctgtgtcaagcgcacgggctcacacctgaacaagtagtggccatcgcttccaacattggtggcaagcaggccctggaaactgtacagcggttgttgcctgtcctctgccaagcgcatgggcttaccccggagcaagtggtcgctatcgcttctaacaacggtgggaagcaggcgctggaaacagtgcagagactgcttccggtgctgtgccaggcacatggcctgacgccggaacaagtggtggcgattgcatctaacggaggcgggaagcaggcgctggagacagtgcaacggctgctccctgttctatgccaagctcatggactcacccccgagcaagtcgtcgccattgcgtcccacgatggcggaaagcaggctttggaaactgtgcagcgcttgctgcctgtgctctgtcaggctcacggactcacccccgagcaagtcgtagccatcgcctcaaatatcggaggcaagcaagcgctcgagactgtgcagaggctcctgcctgtgttatgtcaggcacacgggctgacccctgaacaagtcgtggccattgcctccaacggtggcggaaaacaggcgctcgaaacagtccagaggcttttgcccgtgctgtgtcaagctcacggcctgactccggagcaagtggtcgccattgcatcccatgatggcggtaaacaagcattggaaacggtccaacgcctgctgccggtcctgtgccaggcccatggtctgactcccgaacaggtcgtggctatcgcctccaacatcggtggaaaacaagccctggagactgtacagaggctgctgcccgtgctatgtcaagcacatggacttacgcctgaacaagtggtggcaattgcatcacacgacggagggaagcaagcccttgaaaccgtgcaacgacttctgcccgtcctgtgtcaggcacacggtctgactcccgaacaggtggtggccatcgccagcaatatcgggggcaagcaggcgttggaaactgtgcaaaggctcctcccggtgctgtgccaagcccatggattgactccagaacaggtggtcgctatcgcatcacatgacggtggaaagcaagccctcgaaactgtgcagcggctcctccctgtgttgtgccaggcgcacggtctgactcccgagcaagtcgtggcaatcgcctcgaacggagggggcaaacaagctctcgaaactgttcaaagactgctccccgtgctctgtcaagctcacggcctcactcctgaacaggtagtcgcgattgcctccaacggggggggaaagcaggccttggagacagtccagcggctcttgccggtcttgtgccaagcacacggattgactccagaacaggtggtcgcaatcgccagccacgacggggggaagcaagccttggaaaccgttcagcggctactccctgtgctgtgtcaagcccacggtctgaccccagaacaagtggtggctatcgcgtcgcacgatggaggcaaacaggccctcgagactgtccagagactcctgccagtcctctgccaggctcatgggctgactcctgagcaagttgtggccatagcatccaacggcggaggaaagcaagctctggaaaccgtacagagacttctcccggtgttgtgtcaggcccacggacttactccggaacaggttgtcgccatcgcgtccaacataggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtcgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagaggcctgccgctaccaagaaagcaggacaagccaagaagaagaagggatcctacccgtatgacgtgcccgactacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 287 - DNA, 3125 bp, synthetic construct
  source 1..3125: mol_type = other DNA
aggaaataagagagaaaagaagagtaagaagaaatataagagccaccatggccccaaagaagaagcggaaagtcggaggttcaggcggctcgcatatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacaacggcggcaagcaggctctggaaaccgtccagcgcctccttcctgtcctgtgtcaagcgcacgggctcacacctgaacaagtagtggccatcgcttccaacattggtggcaagcaggccctggaaactgtacagcggttgttgcctgtcctctgccaagcgcatgggcttaccccggagcaagtggtcgctatcgcttctcatgatggtgggaagcaggcgctggaaacagtgcagagactgcttccggtgctgtgccaggcacatggcctgacgccggaacaagtggtggcgattgcatctaacggaggcgggaagcaggcgctggagacagtgcaacggctgctccctgttctatgccaagctcatggactcacccccgagcaagtcgtcgccattgcgtcccacgacggaggcaaacaggcactggaaacggtgcaaagactgttgcctgtactgtgccaggctcatggccttactcccgaacaggtggtggcaattgcgagtaacatcggcggaaagcaggctttggaaactgtgcagcgcttgctgcctgtgctctgtcaggctcacggactcacccccgagcaagtcgtagccatcgcctcaaataacggaggcaagcaagcgctcgagactgtgcagaggctcctgcctgtgttatgtcaggcacacgggctgacccctgaacaagtcgtggccattgcctccaacaacggcggaaaacaggcgctcgaaacagtccagaggcttttgcccgtgctgtgtcaagctcacggcctgactccggagcaagtggtcgccattgcatccaatggcggcggtaaacaagcattggaaacggtccaacgcctgctgccggtcctgtgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcaaacgggggagggaagcaagcccttgaaaccgtgcaaaggctcctcccggtgctgtgccaagcccatggattgactccagaacaggtggtcgctatcgcatcaaacaacggtggaaagcaagccctcgaaactgtgcagcggctcctccctgtgttgtgccaggcgcacggtctgactcccgagcaagtcgtggcaatcgcctcgcacgatgggggcaaacaagctctcgaaactgttcaaagactgctccccgtgctctgtcaagctcacggcctcactcctgaacaggtagtcgcgattgcctcccatgacgggggaaagcaggccttggagacagtccagcggctcttgccggtcttgtgccaagcacacggattgactccagaacaggtggtcgcaatcgccagccatgatggggggaagcaagccttggaaaccgttcagcggctactccctgtgctgtgtcaagcccacggtctgaccccagaacaagtggtggctatcgcgtcgaacggaggaggcaaacaggccctcgagactgtccagagactcctgccagtcctctgccaggctcatgggctgactcctgagcaagttgtggccatagcatccaacaatggaggaaagcaagctctggaaaccgtacagagacttctcccggtgttgtgtcaggcccacggacttactccggaacaggttgtcgccatcgcgtccaacatcggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtcgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagaggcctgccgctaccaagaaagcaggacaagccaagaagaagaagggatcctacccgtatgacgtgcccgactacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 288 - DNA, 3125 bp, synthetic construct
  source 1..3125: mol_type = other DNA
aggaaataagagagaaaagaagagtaagaagaaatataagagccaccatggccccaaagaagaagcggaaagtcggaggttcaggcggctcgcatatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctcaccccggaacaggtggtggcgattgcttcccacgacggtggcaagcaggccttggaaaccgtccaacggcttttgccggtgctttgccaggcccatggactgacccccgaacaagtcgttgccattgctagtcacgacggcggcaagcaggctctggaaaccgtccagcgcctccttcctgtcctgtgtcaagcgcacgggctcacacctgaacaagtagtggccatcgcttccaacggcggtggcaagcaggccctggaaactgtacagcggttgttgcctgtcctctgccaagcgcatgggcttaccccggagcaagtggtcgctatcgcttctaataatggtgggaagcaggcgctggaaacagtgcagagactgcttccggtgctgtgccaggcacatggcctgacgccggaacaagtggtggcgattgcatctaacattggcgggaagcaggcgctggagacagtgcaacggctgctccctgttctatgccaagctcatggactcacccccgagcaagtcgtcgccattgcgtccaacaacggaggcaaacaggcactggaaacggtgcaaagactgttgcctgtactgtgccaggctcatggccttactcccgaacaggtggtggcaattgcgagtaacaacggtggaaagcaggcgctcgaaactgtccagagattgctgcctgtgctgtgtcaggcacacggactgaccccagaacaagtggtggccatcgcgagccatgacggcggaaagcaggctttggaaactgtgcagcgcttgctgcctgtgctctgtcaggctcacggactcacccccgagcaagtcgtagccatcgcctcaaatatcggaggcaagcaagcgctcgagactgtgcagaggctcctgcctgtgttatgtcaggcacacgggctgacccctgaacaagtcgtggccattgcctccaacggtggcggaaaacaggcgctcgaaacagtccagaggcttttgcccgtgctgtgtcaagctcacggcctgactccggagcaagtggtcgccattgcatccaataacggcggtaaacaagcattggaaacggtccaacgcctgctgccggtcctgtgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcacacgacggagggaagcaagcccttgaaaccgtgcaaaggctcctcccggtgctgtgccaagcccatggattgactccagaacaggtggtcgctatcgcatcaaacatcggtggaaagcaagccctcgaaactgtgcagcggctcctccctgtgttgtgccaggcgcacggtctgactcccgagcaagtcgtggcaatcgcctcgaacaatgggggcaaacaagctctcgaaactgttcaaagactgctccccgtgctctgtcaagctcacggcctcactcctgaacaggtagtcgcgattgcctcccatgacgggggaaagcaggccttggagacagtccagcggctcttgccggtcttgtgccaagcacacggattgactccagaacaggtggtcgcaatcgccagccatgatggaggaaagcaagctctggaaaccgtacagagacttctcccggtgttgtgtcaggcccacggacttactccggaacaggttgtcgccatcgcgtccaacatcggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtcgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagaggcctgccgctaccaagaaagcaggacaagccaagaagaagaagggatcctacccgtatgacgtgcccgactacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 289 - DNA, 3125 bp, synthetic construct
  source 1..3125: mol_type = other DNA
aggaaataagagagaaaagaagagtaagaagaaatataagagccaccatggccccaaagaagaagcggaaagtcggaggttcaggcggctcgcatatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtcatgacggcggcaagcaggctctggaaaccgtccagcgcctccttcctgtcctgtgtcaagcgcacgggctcacacctgaacaagtagtggccatcgcttccaacggaggtggcaagcaggccctggaaactgtacagcggttgttgcctgtcctctgccaagcgcatgggcttaccccggagcaagtggtcgctatcgcttctaacaacggaggcaagcaagcgctcgagactgtgcagaggctcctgcctgtgttatgtcaggcacacgggctgacccctgaacaagtcgtggccattgcctccaacatcggcggaaaacaggcgctcgaaacagtccagaggcttttgcccgtgctgtgtcaagctcacggcctgactccggagcaagtggtcgccattgcatccaacaacggcggtaaacaagcattggaaacggtccaacgcctgctgccggtcctgtgccaggcccatggtctgactcccgaacaggtcgtggctatcgcctccaacggaggtggtaagcaagcactggaaaccgtgcagcggttgctccccgtgctctgtcaggctcacgggctcactccagagcaggtggtggccattgccagccatgacggtggaaaacaagccctggagactgtacagaggctgctgcccgtgctatgtcaagcacatggacttacgcctgaacaagtggtggcaattgcatcaaacattggagggaagcaagcccttgaaaccgtgcaacgacttctgcccgtcctgtgtcaggcacacggtctgactcccgaacaggtggtggccatcgccagccacgacgggggcaagcaggcgttggaaactgtgcaaaggctcctcccggtgctgtgccaagcccatggattgactccagaacaggtggtcgctatcgcatcacatgacggtggaaagcaagccctcgaaactgtgcagcggctcctccctgtgttgtgccaggcgcacggtctgactcccgagcaagtcgtggcaatcgcctcgaacggagggggcaaacaagctctcgaaactgttcaaagactcttgccggtcttgtgccaagcacacggattgactccagaacaggtggtcgcaatcgccagccacgacggcgggaagcaggcactcgaaactgtccagcgtctgctgcctgtcctctgccaagcccacggcctcactccagaacaggtggtcgcgattgcaagcaacggcggggggaagcaagccttggaaaccgttcagcggctactccctgtgctgtgtcaagcccacggtctgaccccagaacaagtggtggctatcgcgtcgcacgatggaggcaaacaggccctcgagactgtccagagactcctgccagtcctctgccaggctcatgggctgactcctgagcaagttgtggccatagcatccaacggcggcggcaagcaagccctggagactgtgcaacggctgttgccggtcctgtgccaggcccatggtcttacgccggaacaggtcgtggccatcgcgtcaaacaacggtggcaagcaggctctggagacagtgcaacgccttctgcctgtgctttgccaggcccacggcttgacccccgagcaagtggtggccatcgcctcgaataacggaggaaagcaagctctggaaaccgtacagagacttctcccggtgttgtgtcaggcccacggacttactccggaacaggttgtcgccatcgcgtccaacataggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtcgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagaggcctgccgctaccaagaaagcaggacaagccaagaagaagaagggatcctacccgtatgacgtgcccgactacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 290 - DNA, 3125 bp, synthetic construct
  source 1..3125: mol_type = other DNA
aggaaataagagagaaaagaagagtaagaagaaatataagagccaccatggccccaaagaagaagcggaaagtcggaggttcaggcggctcgcatatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtcatgacggcggcaagcaggctctggaaaccgtccagcgcctccttcctgtcctgtgtcaagcgcacgggctcacacctgaacaagtagtggccatcgcttcccatgatggtggcaagcaggccctggaaactgtacagcggttgttgcctgtcctctgccaagcgcatgggcttaccccggagcaagtggtcgctatcgcttctaacggcggcggcaaacaggcactggaaacggtgcaaagactgttgcctgtactgtgccaggctcatggccttactcccgaacaggtggtggcaattgcgagtaacaacggcggaaagcaggctttggaaactgtgcagcgcttgctgcctgtgctctgtcaggctcacggactcacccccgagcaagtcgtagccatcgcctcaaatggaggaggcaagcaagcgctcgagactgtgcagaggctcctgcctgtgttatgtcaggcacacgggctgacccctgaacaagtcgtggccattgcctccaacaacggcggaaaacaggcgctcgaaacagtccagaggcttttgcccgtgctgtgtcaagctcacggcctgactccggagcaagtggtcgccattgcatcccacgacggcggtaaacaagcattggaaacggtccaacgcctgctgccggtcctgtgccaggcccatgggctgactccggaacaagtcgtcgcgattgcgtccaacatcgggggaaagcaagcactcgaaacagtgcagcggctgctgccagtcctgtgccaggcacatggcctgaccccggagcaggtggtggccatcgcgagcaacattggtgggaagcaggcactggaaactgtccaacggctgctgccggtgctatgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcaaacaacggagggaagcaagcccttgaaaccgtgcaaaggctcctcccggtgctgtgccaagcccatggattgactccagaacaggtggtcgctatcgcatcaaacggaggtggaaagcaagccctcgaaactgtgcagcggctcctccctgtgttgtgccaggcgcacggtctgactcccgagcaagtcgtggcaatcgcctcgcacgacggtgggaagcaagctctggagactgtgcagaggctcctgccggtgctttgtcaggcccacgggcttactccggaacaagtggtggccatcgcctccaacggggggggcaaacaggccttggagacagtccagcggctcttgccggtcttgtgccaagcacacggattgactccagaacaggtggtcgcaatcgccagcaatggcggggggaagcaagccttggaaaccgttcagcggctactccctgtgctgtgtcaagcccacggtctgaccccagaacaagtggtggctatcgcgtcgaacgggggaggcaaacaggccctcgagactgtccagagactcctgccagtcctctgccaggctcatgggctgactcctgagcaagttgtggccatagcatccaacaacggaggaaagcaagctctggaaaccgtacagagacttctcccggtgttgtgtcaggcccacggacttactccggaacaggttgtcgccatcgcgtccaacaacggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtcgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagaggcctgccgctaccaagaaagcaggacaagccaagaagaagaagggatcctacccgtatgacgtgcccgactacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 291 - DNA, 3125 bp, synthetic construct
  source 1..3125: mol_type = other DNA
aggaaataagagagaaaagaagagtaagaagaaatataagagccaccatggccccaaagaagaagcggaaagtcggaggttcaggcggctcgcatatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacggtggcggcaagcaggctctggaaaccgtccagcgcctccttcctgtcctgtgtcaagcgcacgggctcacacctgaacaagtagtggccatcgcttccaacaatggtggcaagcaggccctggaaactgtacagcggttgttgcctgtcctctgccaagcgcatgggcttaccccggagcaagtggtcgctatcgcttctcatgatggtgggaagcaggcgctggaaacagtgcagagactgcttccggtgctgtgccaggcacatggcctgacgccggaacaagtggtggcgattgcatctcacgatggcgggaagcaggcgctggagacagtgcaacggctgctccctgttctatgccaagctcatggactcacccccgagcaagtcgtcgccattgcgtccaacggaggaggcaaacaggcactggaaacggtgcaaagactgttgcctgtactgtgccaggctcatggccttactcccgaacaggtggtggcaattgcgagtcacgacggcggaaagcaggctttggaaactgtgcagcgcttgctgcctgtgctctgtcaggctcacggactcacccccgagcaagtcgtagccatcgcctcaaatatcggaggcaagcaagcgctcgagactgtgcagaggctcctgcctgtgttatgtcaggcacacgggctgacccctgaacaagtcgtggccattgcctccaacaacggcggaaaacaggcgctcgaaacagtccagaggcttttgcccgtgctgtgtcaagctcacggcctgactccggagcaagtggtcgccattgcatccaacaatggcggtaaacaagcattggaaacggtccaacgcctgctgccggtcctgtgccaggcccatggtctgactcccgaacaggtcgtggctatcgcctccaacatcggagggaagcaagcccttgaaaccgtgcaaaggctcctcccggtgctgtgccaagcccatggattgactccagaacaggtggtcgctatcgcatcaaacaacggtggaaagcaagccctcgaaactgtgcagcggctcctccctgtgttgtgccaggcgcacggtctgactcccgagcaagtcgtggcaatcgcctcgaacggagggggcaaacaagctctcgaaactgttcaaagactgctccccgtgctctgtcaagctcacggcctcactcctgaacaggtagtcgcgattgcctcccatgacgggggaaagcaggccttggagacagtccagcggctcttgccggtcttgtgccaagcacacggattgactccagaacaggtggtcgcaatcgccagcaatatcggcggcaagcaagccctggagactgttcagaggctcttgcctgtgctctgccaggcccatggcttgacacctgaacaggtggtggccatcgcgagcaacggtggggggaagcaagccttggaaaccgttcagcggctactccctgtgctgtgtcaagcccacggtctgaccccagaacaagtggtggctatcgcgtcgaacaacggaggcaaacaggccctcgagactgtccagagactcctgccagtcctctgccaggctcatgggctgactcctgagcaagttgtggccatagcatccaacaacggaggaaagcaagctctggaaaccgtacagagacttctcccggtgttgtgtcaggcccacggacttactccggaacaggttgtcgccatcgcgtccaacaacggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtcgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagaggcctgccgctaccaagaaagcaggacaagccaagaagaagaagggatcctacccgtatgacgtgcccgactacgcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 292 - DNA, 18 bp, synthetic construct
  source 1..18: mol_type = other DNA
tactgaagctccacaatt

SEQ ID NO: 293 - DNA, 18 bp, synthetic construct
  source 1..18: mol_type = other DNA
actccgtatttgataagg

SEQ ID NO: 294 - DNA, 18 bp, synthetic construct
  source 1..18: mol_type = other DNA
gagtcatcacacttccta

SEQ ID NO: 295 - DNA, 18 bp, synthetic construct
  source 1..18: mol_type = other DNA
gactcaggtttgccctga

SEQ ID NO: 296 - DNA, 18 bp, synthetic construct
  source 1..18: mol_type = other DNA
cctgaggcatgtcagcca

SEQ ID NO: 297 - DNA, 18 bp, synthetic construct
  source 1..18: mol_type = other DNA
ctgagtcacctctctgga

SEQ ID NO: 298 - DNA, 18 bp, synthetic construct
  source 1..18: mol_type = other DNA
cctgtgcaatgtctttgg

SEQ ID NO: 299 - DNA, 18 bp, synthetic construct
  source 1..18: mol_type = other DNA
tgcctcaggagtcatggg

SEQ ID NO: 300 - protein, 1362 aa, synthetic construct
MAPKKKRKVGIHGVPAAGSSGSLEPGEKPYKCPECGKSFSTSGNLVRHQRTHTGEKPYKCPECGKSFSRADNLTEHQRTHTGEKPYKCPECGKSFSQSGDLRRHQRTHTGEKPYKCPECGKSFSHKNALQNHQRTHTGEKPYKCPECGKSFSDPGNLVRHQRTHTGEKPYKCPECGKSFSQRANLRAHQRTHTGEKPYKCPECGKSFSDCRDLARHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSGSATNFSLLKQAGDVEENPGPTSAGKLGSGEGRGSLLTCGDVEENPGPLEGSSGSGSPKKKRKVGIHGVPAAGSSGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSGS

SEQ ID NO: 301 - RNA, 4364 nt, synthetic construct
  source 1..4364: mol_type = other RNA
gggaaataagagagaaaagaagagtaagaagaaatataagagccaccatggccccaaagaagaagagaaaggtcggaattcatggcgtgcccgcagccggcagcagcggttcccttgagccgggagagaaaccgtacaagtgtcctgaatgtggaaagagtttctccacctcgggcaacctcgtgcgccaccagaggacacacaccggcgagaagccctacaagtgccccgagtgcggtaaatccttttcacgcgcggacaatctgactgaacaccagagaacgcataccggggagaagccctacaagtgcccggaatgcggtaaaagcttctcccagtccggggacttaaggcggcatcaaagaactcatactggagagaagccgtacaaatgccctgaatgcggaaagtccttctcccacaagaacgccctgcagaaccatcaacggacacatactggcgaaaaaccatacaagtgtccagaatgtgggaagtcattttccgacccggggaacctggtccggcaccaacgcacacacactggtgaaaaaccctacaaatgtccggagtgtgggaagagttttagccagagagccaacctgagagctcatcagcggacccatacaggagagaagccttacaaatgccccgaatgtggaaaatcattcagcgactgccgcgaccttgctaggcaccagcgcactcataccggaaaaaagacctccgccagcggatcaggaggtggatcaggcggagatgccaagtcactgaccgcatggtcccggacccttgtgaccttcaaggacgtgttcgtggacttcaccagagaggagtggaagctgctggacaccgcgcaacagattctctaccggaacgtgatgctggagaactacaagaatctggtgtccctgggctaccagctgaccaagccggacgtgatcctccgcctcgaaaagggggaggagccttggctcgtggagcgggaaatccaccaggagactcacccggactccgagactgccttcgaaatcaagtcatcggtgtccggtggaaagcggcctgcagcgactaagaaggccggacaggccaagaagaagaagggctcaggctcagcaaccaacttctccttgctcaaacaagccggggacgtcgaagagaaccccggaccgactagcgccggaaagctgggttccggagagggtcgaggttccctccttacctgtggagatgtggaagaaaacccaggacccctggagggctccagcggtagcggaagcccgaagaagaagcggaaggtcggaatccacggagtgcctgccgctggaagctcgggttcccatatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacggtggcggcaagcaggctctggaaaccgtccagcgcctccttcctgtcctgtgtcaagcgcacgggctcacacctgaacaagtagtggccatcgcttccaacattggtggcaagcaggccctggaaactgtacagcggttgttgcctgtcctctgccaagcgcatgggcttaccccggagcaagtggtcgctatcgcttctcatgatggtgggaagcaggcgctggaaacagtgcagagactgcttccggtgctgtgccaggcacatggcctgacgccggaacaagtggtggcgattgcatctaacggaggcgggaagcaggcgctggagacagtgcaacggctgctccctgttctatgccaagctcatggactcacccccgagcaagtcgtcgccattgcgtccaacaatggaggcaaacaggcactggaaacggtgcaaagactgttgcctgtactgtgccaggctcatggccttactcccgaacaggtggtggcaattgcgagtaacatcggcggaaagcaggctttggaaactgtgcagcgcttgctgcctgtgctctgtcaggctcacggactcacccccgagcaagtcgtagccatcgcctcaaatatcggaggcaagcaagcgctcgagactgtgcagaggctcctgcctgtgttatgtcaggcacacgggctgacccctgaacaagtcgtggccattgcctccaacaacggcggaaaacaggcgctcgaaacagtccagaggcttttgcccgtgctgtgtcaagctcacggcctgactccggagcaagtggtcgccattgcatcccatgatggcggtaaacaagcattggaaacggtccaacgcctgctgccggtcctgtgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcacacgacggagggaagcaagcccttgaaaccgtgcaaaggctcctcccggtgctgtgccaagcccatggattgactccagaacaggtggtcgctatcgcatcacatgacggtggaaagcaagccctcgaaactgtgcagcggctcctccctgtgttgtgccaggcgcacggtctgactcccgagcaagtcgtggcaatcgcctcgaacatcgggggcaaacaagctctcgaaactgttcaaagactgctccccgtgctctgtcaagctcacggcctcactcctgaacaggtagtcgcgattgcctcccatgacgggggaaagcaggccttggagacagtccagcggctcttgccggtcttgtgccaagcacacggattgactccagaacaggtggtcgcaatcgccagcaatatcggggggaagcaagccttggaaaccgttcagcggctactccctgtgctgtgtcaagcccacggtctgaccccagaacaagtggtggctatcgcgtcgaacattggaggcaaacaggccctcgagactgtccagagactcctgccagtcctctgccaggctcatgggctgactcctgagcaagttgtggccatagcatccaacggcggaggaaagcaagctctggaaaccgtacagagacttctcccggtgttgtgtcaggcccacggacttactccggaacaggttgtcgccatcgcgtccaacggtggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtcgcatccggttcaggaggagggtccggaggagctagggatgatgccaagagcttgaccgcctggtcacggactcttgtcactttcaaggatgtctttgtggactttaccagagaagaatggaagctcctggacactgcgcagcagatcctgtacagaaacgtgatgttggaaaactacaaaaacctggtgtcactcgggtaccaactcaccaagcccgacgtcatcctgcgcctggaaaagggcgaagaaccgtggttggtcgagagagaaatccatcaggaaacccaccctgactcggaaaccgccttcgagatcaagtcctcggtgcctagctccggaggaaaaaggcccgccgcaaccaagaaggctgggcaggctaagaaaaagaaggggtccggctcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 302 - RNA, 612 nt, synthetic construct
  source 1..612: mol_type = other RNA
cttgagccgggagagaaaccgtacaagtgtcctgaatgtggaaagagtttctccacctcgggcaacctcgtgcgccaccagaggacacacaccggcgagaagccctacaagtgccccgagtgcggtaaatccttttcacgcgcggacaatctgactgaacaccagagaacgcataccggggagaagccctacaagtgcccggaatgcggtaaaagcttctcccagtccggggacttaaggcggcatcaaagaactcatactggagagaagccgtacaaatgccctgaatgcggaaagtccttctcccacaagaacgccctgcagaaccatcaacggacacatactggcgaaaaaccatacaagtgtccagaatgtgggaagtcattttccgacccggggaacctggtccggcaccaacgcacacacactggtgaaaaaccctacaaatgtccggagtgtgggaagagttttagccagagagccaacctgagagctcatcagcggacccatacaggagagaagccttacaaatgccccgaatgtggaaaatcattcagcgactgccgcgaccttgctaggcaccagcgcactcataccggaaaaaagacctcc

SEQ ID NO: 303 - RNA, 288 nt, synthetic construct
  source 1..288: mol_type = other RNA
gatgccaagtcactgaccgcatggtcccggacccttgtgaccttcaaggacgtgttcgtggacttcaccagagaggagtggaagctgctggacaccgcgcaacagattctctaccggaacgtgatgctggagaactacaagaatctggtgtccctgggctaccagctgaccaagccggacgtgatcctccgcctcgaaaagggggaggagccttggctcgtggagcgggaaatccaccaggagactcacccggactccgagactgccttcgaaatcaagtcatcggtg

SEQ ID NO: 304 - RNA, 2394 nt, synthetic construct
  source 1..2394: mol_type = other RNA
catatggtggacctgagaacccttggatactcccagcagcagcaggagaagattaagcccaaggtccggtcgaccgtcgctcaacaccacgaggccctcgtgggacatggcttcacccacgcacacattgtcgcgctgtcccaacaccctgccgcccttggaactgtggccgtcaagtaccaggatatgatcgccgctctgcctgaagcgacgcatgaagcgattgtgggcgtgggcaagagaggagcaggagcccgagcactggaagcccttctgaccgtcgccggcgaactgaggggtccacctctgcaactggataccgggcagctgctgaagatcgctaagagaggcggagtgaccgccgtcgaagccgtccatgcatggcgcaacgcgctgactggagcaccgctgaacctgacccccgaacaagtcgttgccattgctagtaacggtggcggcaagcaggctctggaaaccgtccagcgcctccttcctgtcctgtgtcaagcgcacgggctcacacctgaacaagtagtggccatcgcttccaacattggtggcaagcaggccctggaaactgtacagcggttgttgcctgtcctctgccaagcgcatgggcttaccccggagcaagtggtcgctatcgcttctcatgatggtgggaagcaggcgctggaaacagtgcagagactgcttccggtgctgtgccaggcacatggcctgacgccggaacaagtggtggcgattgcatctaacggaggcgggaagcaggcgctggagacagtgcaacggctgctccctgttctatgccaagctcatggactcacccccgagcaagtcgtcgccattgcgtccaacaatggaggcaaacaggcactggaaacggtgcaaagactgttgcctgtactgtgccaggctcatggccttactcccgaacaggtggtggcaattgcgagtaacatcggcggaaagcaggctttggaaactgtgcagcgcttgctgcctgtgctctgtcaggctcacggactcacccccgagcaagtcgtagccatcgcctcaaatatcggaggcaagcaagcgctcgagactgtgcagaggctcctgcctgtgttatgtcaggcacacgggctgacccctgaacaagtcgtggccattgcctccaacaacggcggaaaacaggcgctcgaaacagtccagaggcttttgcccgtgctgtgtcaagctcacggcctgactccggagcaagtggtcgccattgcatcccatgatggcggtaaacaagcattggaaacggtccaacgcctgctgccggtcctgtgccaggcccatggtctgactcccgaacaggtcgtggctatcgcctccaacggaggtggaaaacaagccctggagactgtacagaggctgctgcccgtgctatgtcaagcacatggacttacgcctgaacaagtggtggcaattgcatcacacgacggagggaagcaagcccttgaaaccgtgcaaaggctcctcccggtgctgtgccaagcccatggattgactccagaacaggtggtcgctatcgcatcacatgacggtggaaagcaagccctcgaaactgtgcagcggctcctccctgtgttgtgccaggcgcacggtctgactcccgagcaagtcgtggcaatcgcctcgaacatcgggggcaaacaagctctcgaaactgttcaaagactgctccccgtgctctgtcaagctcacggcctcactcctgaacaggtagtcgcgattgcctcccatgacgggggaaagcaggccttggagacagtccagcggctcttgccggtcttgtgccaagcacacggattgactccagaacaggtggtcgcaatcgccagcaatatcggggggaagcaagccttggaaaccgttcagcggctactccctgtgctgtgtcaagcccacggtctgaccccagaacaagtggtggctatcgcgtcgaacattggaggcaaacaggccctcgagactgtccagagactcctgccagtcctctgccaggctcatgggctgactcctgagcaagttgtggccatagcatccaacggcggaggaaagcaagctctggaaaccgtacagagacttctcccggtgttgtgtcaggcccacggacttactccggaacaggttgtcgccatcgcgtccaacggtggtggcaggcctgctctggaatccatcgtggcacagttgtcaagaccagatcctgccctggctgcgctcaccaacgaccacttggtggctctggcctgccttggcggtcgccctgccttggatgccgtgaagaagggacttcctcacgccccggcactgattaagcgcaccaatcgcaggatccctgaacggacctcgcacagggtc

SEQ ID NO: 305 - RNA, 288 nt, synthetic construct
  source 1..288: mol_type = other RNA
gatgccaagagcttgaccgcctggtcacggactcttgtcactttcaaggatgtctttgtggactttaccagagaagaatggaagctcctggacactgcgcagcagatcctgtacagaaacgtgatgttggaaaactacaaaaacctggtgtcactcgggtaccaactcaccaagcccgacgtcatcctgcgcctggaaaagggcgaagaaccgtggttggtcgagagagaaatccatcaggaaacccaccctgactcggaaaccgccttcgagatcaagtcctcggtg

SEQ ID NO: 306 - protein, 373 aa, synthetic construct
MAPKKKRKVGIHGVPAAGSSGSLEPGEKPYKCPECGKSFSTSGNLVRHQRTHTGEKPYKCPECGKSFSRADNLTEHQRTHTGEKPYKCPECGKSFSQSGDLRRHQRTHTGEKPYKCPECGKSFSHKNALQNHQRTHTGEKPYKCPECGKSFSDPGNLVRHQRTHTGEKPYKCPECGKSFSQRANLRAHQRTHTGEKPYKCPECGKSFSDCRDLARHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSGSATNFSLLKQAGDVEENPG

SEQ ID NO: 307 - protein, 962 aa, synthetic construct
PLEGSSGSGSPKKKRKVGIHGVPAAGSSGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSGS

SEQ ID NO: 308-311 - skipped (4 entries)

SEQ ID NO: 312 - protein, 954 aa, Mus sp.
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 313 - RNA, 3137 nt, Mus sp.
  source 1..3137: mol_type = genomic RNA
gggaaataagagagaaaagaagagtaagaagaaatataagagccaccatggccccaaagaagaagcggaaagtcggaggttcaggcggctcgcacatggtcgacctgaggaccctcggatactcgcagcagcagcaggaaaagatcaagcctaaggtccgcagcaccgtggctcagcatcatgaggcccttgtgggacacgggttcacccacgcccatattgtggccctgtcccaacacccggctgcactgggcaccgtggccgtgaagtaccaggacatgatcgccgccctgcccgaggcgacccacgaggccatcgtgggagtgggaaagcgcggcgccggtgccagagcactggaagcgctgctgactgtggcgggagagctgaggggacctccactccaattggataccgggcagctcctgaagatcgccaagcgcggcggagtgaccgccgtggaagccgtgcacgcctggcggaacgcgctgactggggccccgctgaacctgaccccagaacaggttgtggctatcgcctccaacaacggcggaaaacaggccttggagactgtgcagcggcttctgccggtgctttgtcaagcccacggcctgaccccggaacaggtcgtggccatcgcctcaaacggcggcgggaagcaggctctcgaaaccgtgcaacggctcctgcccgtgctgtgccaagctcatggtctgacacctgaacaagtggtggcgattgcgtcccacgacgggggcaaacaggccctcgaaactgtccagcggctgctgcccgtgttgtgccaagcacacggactcacgcccgaacaagtcgtcgcgattgcaagcaatggtggtggaaagcaggccctggagactgtccagagactcctgcccgtgctgtgccaggctcacgggctgaccccggaacaagtcgtggccattgccagcaacatcggcggcaaacaggctctggagactgtgcagcgcctgctgccagtgctgtgtcaggcgcacggactgacccccgaacaggtcgtggcaatcgcatcaaacatcggaggaaaacaggccttggaaaccgtccagcggctgttgcccgtgctttgccaagctcatggactcacccctgagcaagtcgtcgcgattgcctccaacggtggaggaaagcaagcccttgagactgtccagaggctgctgccggtgttatgccaggcccacggtctgaccccagaacaggtggtggccatcgccagccatgacggtggcaaacaagccctcgaaacagtgcagagactgctcccggtcctgtgccaagcccacggcctcaccccggagcaagtggtggctatcgcctcgcacgatggagggaagcaagcgctggaaaccgtgcagcggttgctgcccgtcctgtgtcaagcgcacggcctgactccggaacaggtagtcgccattgcgtccaacggcggcgggaagcaggcgctcgaaacggtgcagcggctgctgcctgtgctctgccaagcacacggacttacccctgaacaagtggtcgccatcgcctccaatggtggcggaaagcaggccctggaaactgtgcaacgcctcctgcccgtgttgtgccaggcgcatgggctcactcctgaacaggttgtcgcaatcgcttccaacaacggcgggaaacaggcactggaaaccgtgcagcggctgttgccagtgttgtgccaagcgcacggcctgactcctgaacaagtggtggccattgcgtcgcggcatggtggcaaacaggccctcgaaactgtgcaacggctgctgccggtgttgtgtcaggcgcacggactgaccccggagcaagtcgtcgctatcgcgagcagacacggcggcaagcaggccctggaaaccgtgcaacgccttctgcccgtgctgtgccaggcgcatggtctgacccctgagcaagtggtcgccatcgcatccaacatcgggggcaaacaagccctggagactgtgcagaggctcctgccggtgctctgccaagcccacgggctcacccccgaacaagtcgtggccatcgcctccaacaacggcgggaagcaggctctggaaacggtgcagagactgctccctgtgctgtgtcaggcccacggactcacccctgaacaggttgtcgcaatcgcgtcgaatggagggggaaagcaggcactggagacagtgcagcgcctgctccccgtgctttgccaagctcacggacttaccccggaacaggtcgtggcaatcgcctcaaacaacggtggacgcccggccctggagagcatcgtggcccagctctcccgccccgatccggccctggctgccctcactaacgaccacctggtcgccctggcctgcctgggcggacggccagccctggacgccgtgaagaagggactgccccacgcccccgccctgattaagcggaccaaccgcaggattcctgagagaacctcccatcgggtggcatctgggagcggaggaggatccggcggagccagagacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgccttcctccggtggaaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 314 - skipped

SEQ ID NO: 315 - RNA, 2394 nt, Mus sp.
  source 1..2394: mol_type = genomic RNA
cacatggtcgacctgaggaccctcggatactcgcagcagcagcaggaaaagatcaagcctaaggtccgcagcaccgtggctcagcatcatgaggcccttgtgggacacgggttcacccacgcccatattgtggccctgtcccaacacccggctgcactgggcaccgtggccgtgaagtaccaggacatgatcgccgccctgcccgaggcgacccacgaggccatcgtgggagtgggaaagcgcggcgccggtgccagagcactggaagcgctgctgactgtggcgggagagctgaggggacctccactccaattggataccgggcagctcctgaagatcgccaagcgcggcggagtgaccgccgtggaagccgtgcacgcctggcggaacgcgctgactggggccccgctgaacctgaccccagaacaggttgtggctatcgcctccaacaacggcggaaaacaggccttggagactgtgcagcggcttctgccggtgctttgtcaagcccacggcctgaccccggaacaggtcgtggccatcgcctcaaacggcggcgggaagcaggctctcgaaaccgtgcaacggctcctgcccgtgctgtgccaagctcatggtctgacacctgaacaagtggtggcgattgcgtcccacgacgggggcaaacaggccctcgaaactgtccagcggctgctgcccgtgttgtgccaagcacacggactcacgcccgaacaagtcgtcgcgattgcaagcaatggtggtggaaagcaggccctggagactgtccagagactcctgcccgtgctgtgccaggctcacgggctgaccccggaacaagtcgtggccattgccagcaacatcggcggcaaacaggctctggagactgtgcagcgcctgctgccagtgctgtgtcaggcgcacggactgacccccgaacaggtcgtggcaatcgcatcaaacatcggaggaaaacaggccttggaaaccgtccagcggctgttgcccgtgctttgccaagctcatggactcacccctgagcaagtcgtcgcgattgcctccaacggtggaggaaagcaagcccttgagactgtccagaggctgctgccggtgttatgccaggcccacggtctgaccccagaacaggtggtggccatcgccagccatgacggtggcaaacaagccctcgaaacagtgcagagactgctcccggtcctgtgccaagcccacggcctcaccccggagcaagtggtggctatcgcctcgcacgatggagggaagcaagcgctggaaaccgtgcagcggttgctgcccgtcctgtgtcaagcgcacggcctgactccggaacaggtagtcgccattgcgtccaacggcggcgggaagcaggcgctcgaaacggtgcagcggctgctgcctgtgctctgccaagcacacggacttacccctgaacaagtggtcgccatcgcctccaatggtggcggaaagcaggccctggaaactgtgcaacgcctcctgcccgtgttgtgccaggcgcatgggctcactcctgaacaggttgtcgcaatcgcttccaacaacggcgggaaacaggcactggaaaccgtgcagcggctgttgccagtgttgtgccaagcgcacggcctgactcctgaacaagtggtggccattgcgtcgcggcatggtggcaaacaggccctcgaaactgtgcaacggctgctgccggtgttgtgtcaggcgcacggactgaccccggagcaagtcgtcgctatcgcgagcagacacggcggcaagcaggccctggaaaccgtgcaacgccttctgcccgtgctgtgccaggcgcatggtctgacccctgagcaagtggtcgccatcgcatccaacatcgggggcaaacaagccctggagactgtgcagaggctcctgccggtgctctgccaagcccacgggctcacccccgaacaagtcgtggccatcgcctccaacaacggcgggaagcaggctctggaaacggtgcagagactgctccctgtgctgtgtcaggcccacggactcacccctgaacaggttgtcgcaatcgcgtcgaatggagggggaaagcaggcactggagacagtgcagcgcctgctccccgtgctttgccaagctcacggacttaccccggaacaggtcgtggcaatcgcctcaaacaacggtggacgcccggccctggagagcatcgtggcccagctctcccgccccgatccggccctggctgccctcactaacgaccacctggtcgccctggcctgcctgggcggacggccagccctggacgccgtgaagaagggactgccccacgcccccgccctgattaagcggaccaaccgcaggattcctgagagaacctcccatcgggtg

SEQ ID NO: 316 - skipped

SEQ ID NO: 317 - RNA, 288 nt, Mus sp.
  source 1..288: mol_type = genomic RNA
gacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtg

SEQ ID NO: 318 - DNA, 18 bp, Mus sp.
  source 1..18: mol_type = genomic DNA
gtctaatccttgggagtg

SEQ ID NO: 319 - protein, 954 aa, Mus sp.
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGRPALESIVAQLSRPDPALAALTNDHLVALACLGGRPALDAVKKGLPHAPALIKRTNRRIPERTSHRVASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 320 - RNA, 3137 nt, Mus sp.
  source 1..3137: mol_type = genomic RNA
gggaaataagagagaaaagaagagtaagaagaaatataagagccaccatggccccaaagaagaagcggaaagtcggaggttcaggcggctcgcacatggtcgaccttcggaccctcggatactcgcagcagcagcaagagaagatcaagcccaaggtccgctccaccgtggcccagcaccacgaagccctggtcggccacggtttcactcacgcccacattgtggcgctgtcccagcatcctgccgcactcgggaccgtggctgtgaagtaccaggacatgatcgcggccttgcccgaggccacccatgaagccatcgtgggagtggggaagagaggcgccggagccagggcccttgaggccctgctcaccgtggctggagagctgcggggaccgccgctgcaactggatacgggacagctgctgaagatcgcaaagcgcggtggagtgacagcggtcgaggccgtgcatgcctggcggaacgccctcaccggcgcccctctcaacctgacccctgaacaagtggtcgccatcgcgagccacgacggcgggaaacaggctcttgaaaccgtccaacgcttgctgcccgtgctgtgtcaggcgcatgggctgactccggaacaggttgtggctattgccagcaatggcggcggaaagcaggccttggaaactgtgcagaggctgctgccggtgctctgccaagctcacggtctgacccccgaacaagtcgtggccatcgcatccaacaacggcgggaagcaggcactcgaaaccgtgcagagactgctgccagtgctgtgccaagcccacggactgactccagaacaggtcgtggcgattgcctcaaacggtggcggaaaacaagcgctggagactgtgcagcggctgctcccggtgttgtgtcaagcgcacggcctgacccccgagcaagtggtcgccatcgccagcaacatcggtggcaaacaggcactcgaaaccgtccagcggttgctgccagtgctgtgccaagcccacggacttaccccggaacaagtcgtcgcgattgccagcaacaacggcggcaagcaggctctggaaaccgtgcagagactgctgcccgtgttgtgccaggcgcacggactgacccctgaacaggtcgtggcaattgcctccaatggtggcggcaaacaagctctggagactgtgcagaggctgctgcctgtcctgtgccaggcacacggtctgactcctgaacaagtggtggcgattgcctcaaacatcggagggaagcaggccctggagacagtgcagcgcctgctgccggtgctttgccaagctcatggactgactccagaacaggttgtggccattgcctcccatgacggagggaaacaagccctggaaacggtgcagcggttactcccggtgctctgtcaggcgcatgggctgaccccggagcaagtcgtggctatcgcctccaacatcggcgggaagcaagccctcgaaactgtgcagcggctgctccctgtgctctgccaagcgcatggtctgacgcccgaacaggtggtggccatcgcctcccacgacggaggaaagcaggccttggagactgtccaacgcctgctccccgtgctgtgtcaggcccacgggcttacccccgaacaagttgtggcaatcgcctcgcacgatggcggaaaacaggccctcgaaaccgtgcagcgcctcctcccggtcctgtgccaagcgcatggactgacccccgaacaggtggtcgcaattgcctccaacattggcgggaaacaggctctggagactgtgcagcggctgctgcctgtgctttgccaagctcacggactcacgccggaacaagtcgtggccatcgcgtccaatatcggcggaaagcaggccctcgaaaccgtccagagacttctcccggtgctctgtcaagcgcacgggctgaccccagaacaggttgtggccattgcgtccaacaacggaggaaaacaggccctggagacagtgcagagactgctgccggtgttgtgccaggcgcacggattgacccctgagcaagtggtggcaatcgcctcaaacggcggcggaaaacaagccctggaaactgtgcaacggctgctccctgtgctgtgccaggcacatggtctgaccccggaacaggtcgtggcgattgcctcgaacaacggcgggaagcaggcacttgagactgtccagaggctgctccccgtgctgtgtcaggcccacggactgactcccgaacaagtggtcgccatcgcctcgcgccacggcgggcgcccagccctggagagcatcgtggcccagctgtcccggcccgaccctgccctggctgccctgaccaacgaccacttggtggccctcgcctgcctgggtggcagaccggccctggatgctgtgaagaagggtctgcctcatgcccccgccctgatcaagcggaccaaccggcgcatccccgagaggaccagccaccgggtggcatctgggagcggaggaggatccggcggagccagagacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgccttcctccggtggaaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 321 - skipped

SEQ ID NO: 322 - RNA, 2394 nt, Mus sp.
  source 1..2394: mol_type = genomic RNA
cacatggtcgaccttcggaccctcggatactcgcagcagcagcaagagaagatcaagcccaaggtccgctccaccgtggcccagcaccacgaagccctggtcggccacggtttcactcacgcccacattgtggcgctgtcccagcatcctgccgcactcgggaccgtggctgtgaagtaccaggacatgatcgcggccttgcccgaggccacccatgaagccatcgtgggagtggggaagagaggcgccggagccagggcccttgaggccctgctcaccgtggctggagagctgcggggaccgccgctgcaactggatacgggacagctgctgaagatcgcaaagcgcggtggagtgacagcggtcgaggccgtgcatgcctggcggaacgccctcaccggcgcccctctcaacctgacccctgaacaagtggtcgccatcgcgagccacgacggcgggaaacaggctcttgaaaccgtccaacgcttgctgcccgtgctgtgtcaggcgcatgggctgactccggaacaggttgtggctattgccagcaatggcggcggaaagcaggccttggaaactgtgcagaggctgctgccggtgctctgccaagctcacggtctgacccccgaacaagtcgtggccatcgcatccaacaacggcgggaagcaggcactcgaaaccgtgcagagactgctgccagtgctgtgccaagcccacggactgactccagaacaggtcgtggcgattgcctcaaacggtggcggaaaacaagcgctggagactgtgcagcggctgctcccggtgttgtgtcaagcgcacggcctgacccccgagcaagtggtcgccatcgccagcaacatcggtggcaaacaggcactcgaaaccgtccagcggttgctgccagtgctgtgccaagcccacggacttaccccggaacaagtcgtcgcgattgccagcaacaacggcggcaagcaggctctggaaaccgtgcagagactgctgcccgtgttgtgccaggcgcacggactgacccctgaacaggtcgtggcaattgcctccaatggtggcggcaaacaagctctggagactgtgcagaggctgctgcctgtcctgtgccaggcacacggtctgactcctgaacaagtggtggcgattgcctcaaacatcggagggaagcaggccctggagacagtgcagcgcctgctgccggtgctttgccaagctcatggactgactccagaacaggttgtggccattgcctcccatgacggagggaaacaagccctggaaacggtgcagcggttactcccggtgctctgtcaggcgcatgggctgaccccggagcaagtcgtggctatcgcctccaacatcggcgggaagcaagccctcgaaactgtgcagcggctgctccctgtgctctgccaagcgcatggtctgacgcccgaacaggtggtggccatcgcctcccacgacggaggaaagcaggccttggagactgtccaacgcctgctccccgtgctgtgtcaggcccacgggcttacccccgaacaagttgtggcaatcgcctcgcacgatggcggaaaacaggccctcgaaaccgtgcagcgcctcctcccggtcctgtgccaagcgcatggactgacccccgaacaggtggtcgcaattgcctccaacattggcgggaaacaggctctggagactgtgcagcggctgctgcctgtgctttgccaagctcacggactcacgccggaacaagtcgtggccatcgcgtccaatatcggcggaaagcaggccctcgaaaccgtccagagacttctcccggtgctctgtcaagcgcacgggctgaccccagaacaggttgtggccattgcgtccaacaacggaggaaaacaggccctggagacagtgcagagactgctgccggtgttgtgccaggcgcacggattgacccctgagcaagtggtggcaatcgcctcaaacggcggcggaaaacaagccctggaaactgtgcaacggctgctccctgtgctgtgccaggcacatggtctgaccccggaacaggtcgtggcgattgcctcgaacaacggcgggaagcaggcacttgagactgtccagaggctgctccccgtgctgtgtcaggcccacggactgactcccgaacaagtggtcgccatcgcctcgcgccacggcgggcgcccagccctggagagcatcgtggcccagctgtcccggcccgaccctgccctggctgccctgaccaacgaccacttggtggccctcgcctgcctgggtggcagaccggccctggatgctgtgaagaagggtctgcctcatgcccccgccctgatcaagcggaccaaccggcgcatccccgagaggaccagccaccgggtg

SEQ ID NO: 323 - skipped

SEQ ID NO: 324 - RNA, 288 nt, Mus sp.; identical to SEQ ID NO: 317
  source 1..288: mol_type = genomic RNA

SEQ ID NO: 325 - DNA, 18 bp, Mus sp.
  source 1..18: mol_type = genomic DNA
ctgtagtacaccaagtgg

SEQ ID NO: 326 - protein, 954 aa, Mus sp.
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 327 - RNA, 3137 nt, Mus sp.
  source 1..3137: mol_type = genomic RNA
gggaaataagagagaaaagaagagtaagaagaaatataagagccaccatggccccaaagaagaagcggaaagtcggaggttcaggcggctcgcacatggtggacctcaggaccctgggatacagccagcagcagcaggaaaagatcaagcccaaggtccgcagcacagtggctcagcaccatgaggcccttgtgggacacggattcacccacgcgcacatcgtggcactgtcccaacaccctgcggccctggggaccgtggccgtgaagtaccaggatatgatcgccgctctgcccgaggccacccatgaagccatcgtgggagtgggaaagcgcggagcgggcgccagagcccttgaggccctgctcactgtggccggcgaactgcggggtcctccgctgcaattggacaccgggcagctcctgaagattgctaagcgcggtggagtgaccgccgtcgaggccgtgcatgcgtggcggaacgccctcactggtgccccgctgaacctgaccccagaacaagttgtggccatcgcatccaacaacggcgggaagcaggccctcgaaaccgtgcagcggctgctgccagtgctgtgtcaggcccacgggctgaccccggaacaggtcgtggcaatcgcctccaatggcggcgggaaacaggcccttgagactgtccagagactgctcccggtcctgtgccaagcacacggactgacgcccgaacaagtggtggctattgcctcaaacggcggcggaaagcaggctctggaaaccgtccagaggctcctgcccgtgctctgccaagcgcacggcttgactcctgaacaagtcgtggcgattgcgtcgaacatcggtggaaaacaagccctggagactgtgcagcggttgctgcccgtgctgtgtcaggcgcacgggctcacccccgaacaggtcgtggccatcgcctcaaacaacggcggaaagcaggccctggaaactgtgcaacgcctgctgcctgtgttgtgtcaggcccacggcctcactcccgaacaagttgtggctatcgcctcgcggcacggagggaagcaagccctggagactgtccagagactgctgcccgtgctttgtcaagcccacggactcacccctgagcaagtggtggccattgcatccaatggtggtggcaaacaggctctcgaaacagtgcagagactcctccccgtgctctgccaagcgcacggactgactcctgaacaagtagtcgccattgcctcccacgatggcgggaaacaggccctcgaaactgtccagcggctgttgccggtgttgtgccaggcacatggtctgaccccggagcaagtcgtggctattgcgtccaacggcggaggaaagcaagccttggagactgtgcagcgcctgcttcctgtgctttgccaagctcacggacttaccccggaacaagtcgtcgcaatcgccagccatgacggcgggaagcaggctctggaaaccgtgcagcggctgctgccagtgctgtgccaggcgcatggactgaccccagaacaagtggtcgccatcgcgagccacgacggcggcaagcaggccttagaaaccgtccagaggctgctgccggtcctgtgccaagcacacggtctgacgccggaacaggtggtcgcgattgcctccaacatcgggggaaaacaggcgctggagaccgtgcagagactcctgcctgtgttatgccaagcccacggcctgactcccgaacaagtcgtcgccattgcgtcacacgacggcgggaagcaggcccttgagacagtgcagcgactcctgcccgtgctgtgccaggctcatggactcaccccggaacaggttgtggccatcgcctccaatggtggcggcaaacaggccctcgaaaccgtccagcggctcctcccggtcctgtgccaagctcatggattgacccctgagcaagtcgtggcaatcgcctcgaacaacggcggaaagcaggccttggagactgtgcagaggctgctgcctgtgctgtgtcaggcccacggactgaccccggagcaagtggtggcgattgcctccaacatcggcggaaaacaagccctggaaaccgtgcagcgccttctgcccgtgttgtgccaggcgcacggtctgacccctgaacaagtggtcgccatcgcgagcaacggagggggaaaacaggctctggaaactgtgcagcggctgctcccggtgctctgtcaagcacacgggctgaccccagaacaggtcgtggccattgcgagcaacaacggtggacgccctgcccttgagtccatcgtggcccagctgtcgaggcccgacccggccctggctgcgctgactaacgaccacctggtggccctcgcctgcctgggcgggcggcccgccctggatgcagtgaagaagggactgccgcatgcaccagccctgatcaagcggaccaaccgcagaatccccgagcgcacgtcccaccgggtggcatctgggagcggaggaggatccggcggagccagagacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgccttcctccggtggaaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 328 - skipped

SEQ ID NO: 329 - RNA, 2394 nt, Mus sp.
  source 1..2394: mol_type = genomic RNA
cacatggtggacctcaggaccctgggatacagccagcagcagcaggaaaagatcaagcccaaggtccgcagcacagtggctcagcaccatgaggcccttgtgggacacggattcacccacgcgcacatcgtggcactgtcccaacaccctgcggccctggggaccgtggccgtgaagtaccaggatatgatcgccgctctgcccgaggccacccatgaagccatcgtgggagtgggaaagcgcggagcgggcgccagagcccttgaggccctgctcactgtggccggcgaactgcggggtcctccgctgcaattggacaccgggcagctcctgaagattgctaagcgcggtggagtgaccgccgtcgaggccgtgcatgcgtggcggaacgccctcactggtgccccgctgaacctgaccccagaacaagttgtggccatcgcatccaacaacggcgggaagcaggccctcgaaaccgtgcagcggctgctgccagtgctgtgtcaggcccacgggctgaccccggaacaggtcgtggcaatcgcctccaatggcggcgggaaacaggcccttgagactgtccagagactgctcccggtcctgtgccaagcacacggactgacgcccgaacaagtggtggctattgcctcaaacggcggcggaaagcaggctctggaaaccgtccagaggctcctgcccgtgctctgccaagcgcacggcttgactcctgaacaagtcgtggcgattgcgtcgaacatcggtggaaaacaagccctggagactgtgcagcggttgctgcccgtgctgtgtcaggcgcacgggctcacccccgaacaggtcgtggccatcgcctcaaacaacggcggaaagcaggccctggaaactgtgcaacgcctgctgcctgtgttgtgtcaggcccacggcctcactcccgaacaagttgtggctatcgcctcgcggcacggagggaagcaagccctggagactgtccagagactgctgcccgtgctttgtcaagcccacggactcacccctgagcaagtggtggccattgcatccaatggtggtggcaaacaggctctcgaaacagtgcagagactcctccccgtgctctgccaagcgcacggactgactcctgaacaagtagtcgccattgcctcccacgatggcgggaaacaggccctcgaaactgtccagcggctgttgccggtgttgtgccaggcacatggtctgaccccggagcaagtcgtggctattgcgtccaacggcggaggaaagcaagccttggagactgtgcagcgcctgcttcctgtgctttgccaagctcacggacttaccccggaacaagtcgtcgcaatcgccagccatgacggcgggaagcaggctctggaaaccgtgcagcggctgctgccagtgctgtgccaggcgcatggactgaccccagaacaagtggtcgccatcgcgagccacgacggcggcaagcaggccttagaaaccgtccagaggctgctgccggtcctgtgccaagcacacggtctgacgccggaacaggtggtcgcgattgcctccaacatcgggggaaaacaggcgctggagaccgtgcagagactcctgcctgtgttatgccaagcccacggcctgactcccgaacaagtcgtcgccattgcgtcacacgacggcgggaagcaggcccttgagacagtgcagcgactcctgcccgtgctgtgccaggctcatggactcaccccggaacaggttgtggccatcgcctccaatggtggcggcaaacaggccctcgaaaccgtccagcggctcctcccggtcctgtgccaagctcatggattgacccctgagcaagtcgtggcaatcgcctcgaacaacggcggaaagcaggccttggagactgtgcagaggctgctgcctgtgctgtgtcaggcccacggactgaccccggagcaagtggtggcgattgcctccaacatcggcggaaaacaagccctggaaaccgtgcagcgccttctgcccgtgttgtgccaggcgcacggtctgacccctgaacaagtggtcgccatcgcgagcaacggagggggaaaacaggctctggaaactgtgcagcggctgctcccggtgctctgtcaagcacacgggctgaccccagaacaggtcgtggccattgcgagcaacaacggtggacgccctgcccttgagtccatcgtggcccagctgtcgaggcccgacccggccctggctgcgctgactaacgaccacctggtggccctcgcctgcctgggcgggcggcccgccctggatgcagtgaagaagggactgccgcatgcaccagccctgatcaagcggaccaaccgcagaatccccgagcgcacgtcccaccgggtg

SEQ ID NO: 330 - skipped

SEQ ID NO: 331 - RNA, 288 nt, Mus sp.; identical to SEQ ID NO: 317
  source 1..288: mol_type = genomic RNA

SEQ ID NO: 332 - DNA, 18 bp, Mus sp.
  source 1..18: mol_type = genomic DNA
gttaggtctccactgatg

SEQ ID NO: 333 - protein, 954 aa, Mus sp.
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 334 - RNA, 3137 nt, Mus sp.
  source 1..3137: mol_type = genomic RNA
gggaaataagagagaaaagaagagtaagaagaaatataagagccaccatggccccaaagaagaagcggaaagtcggaggttcaggcggctcgcacatggtcgacttgcggactctgggatactcgcagcagcagcaagagaagatcaagccgaaggtccgctcaaccgtggcccagcaccacgaggccctcgtcggtcatggtttcacccatgcgcacatcgtcgctctgagccagcacccggctgcacttggaaccgtggccgtgaagtaccaagacatgattgccgcgctgcctgaagcaacccacgaggccattgtgggagtggggaagcggggagccggagccagggccctcgaagccctcctgactgtggccggagagctgcgcgggccaccactccaactggacaccggccagctgctgaagatcgccaagcgcgggggcgtgaccgccgtggaagccgtgcatgcctggcggaacgccctgaccggcgctcccctgaatctgactccggaacaggttgtcgccatcgcgagccatgatggcggaaagcaggccctggaaacggtgcagagactgttgcctgtgctgtgccaagcgcacggactcacccccgaacaggtcgtggctatcgcctccaacatcggcggcaaacaggcactcgaaaccgtgcagaggctcctgcccgtgctgtgtcaggcccacggtcttactcccgaacaagtggtggcaatcgcgtccaacaacggcggaaaacaggctcttgagacagtgcagcgcctgctgcctgtgctctgccaagcccacggactgaccccggaacaagtcgtggcgattgcgtccagacacggcgggaagcaggccttggagactgtgcagcggctgctgcccgtgctttgccaggcgcatggcctgaccccggagcaagtcgtcgcaatcgcctcccgccacggcgggaagcaagccttggagactgtccagagactcctgcccgtgctgtgccaggctcacggacttaccccggaacaggtcgtggctatcgcctcaaacatcggagggaagcaggccctcgaaaccgtgcagcggcttctgcctgtgttgtgccaggcccacggtctgacccctgaacaggttgtggccatcgcctccaacatcggcggaaagcaggcactggaaacggtgcagagactgctccccgtgctatgccaagcgcatggactgactccggaacaagtagtcgccatcgccagcaatggtggagggaaacaagccctggagactgtgcagcgcctcctgccggtgctgtgtcaggcgcacggactcacccctgagcaagtggtcgccattgcctcgaacggtggcggcaagcaggctctggaaaccgtccagaggctgctcccggtgctctgccaagcccacggactgaccccagaacaggtggtcgcaattgcgtccaacggcggtggaaaacaagcgctggaaactgtccagcggctgctgccagtgctctgtcaagcgcacggcctgacacctgaacaagtcgtggcaatcgcgtcccacgatgggggaaaacaggccttggaaacggtccagcgcctgctgcccgtgttgtgtcaggctcatgggctgacccccgaacaagtggtggccattgccagccacgacggcggcaaacaggcccttgagaccgtgcagcggttgctgccagtgctttgccaagctcacgggctgaccccggagcaagtcgtcgcaatcgcctcccacgacgggggcaaacaggccctggaaaccgtgcagaggctgctccctgtgttgtgtcaggcccacggtctgactcctgaacaagtcgtggccatcgcttccaacggcggcggaaaacaggcactcgaaactgtgcagcgcctgctgcccgtgctgtgccaggctcatggactgacgccggaacaggttgtcgccatcgcctcgaacaacggtggaaaacaagccctggagactgtccagaggctccttcccgtgctctgccaagcacatggactcaccccggaacaagtggtggcaatcgcgtcccgccacggaggaaagcaggccttggaaacagtgcagcggctgctcccggtgctgtgtcaagcccacggactgaccccagaacaggtcgtggcgattgccagcaacggtggcgggaagcaggcccttgagactgtgcagagactcctgcccgtgttgtgccaggcgcatggcctcacccctgagcaagtggtcgctatcgcgagccacgatggcggcagacccgccctggagtcaattgtggcgcagctgtcccggcctgatccggccctggccgccctgaccaacgaccacctggtggccctcgcctgcctgggcgggcggcctgcgctggacgccgtgaagaagggactgccccacgccccggccctgatcaagagaaccaatcggcgcattcccgagcggacctcgcaccgcgtggcatctgggagcggaggaggatccggcggagccagagacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgccttcctccggtggaaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 335 - skipped

SEQ ID NO: 336 - RNA, 2394 nt, Mus sp.
  source 1..2394: mol_type = genomic RNA
cacatggtcgacttgcggactctgggatactcgcagcagcagcaagagaagatcaagccgaaggtccgctcaaccgtggcccagcaccacgaggccctcgtcggtcatggtttcacccatgcgcacatcgtcgctctgagccagcacccggctgcacttggaaccgtggccgtgaagtaccaagacatgattgccgcgctgcctgaagcaacccacgaggccattgtgggagtggggaagcggggagccggagccagggccctcgaagccctcctgactgtggccggagagctgcgcgggccaccactccaactggacaccggccagctgctgaagatcgccaagcgcgggggcgtgaccgccgtggaagccgtgcatgcctggcggaacgccctgaccggcgctcccctgaatctgactccggaacaggttgtcgccatcgcgagccatgatggcggaaagcaggccctggaaacggtgcagagactgttgcctgtgctgtgccaagcgcacggactcacccccgaacaggtcgtggctatcgcctccaacatcggcggcaaacaggcactcgaaaccgtgcagaggctcctgcccgtgctgtgtcaggcccacggtcttactcccgaacaagtggtggcaatcgcgtccaacaacggcggaaaacaggctcttgagacagtgcagcgcctgctgcctgtgctctgccaagcccacggactgaccccggaacaagtcgtggcgattgcgtccagacacggcgggaagcaggccttggagactgtgcagcggctgctgcccgtgctttgccaggcgcatggcctgaccccggagcaagtcgtcgcaatcgcctcccgccacggcgggaagcaagccttggagactgtccagagactcctgcccgtgctgtgccaggctcacggacttaccccggaacaggtcgtggctatcgcctcaaacatcggagggaagcaggccctcgaaaccgtgcagcggcttctgcctgtgttgtgccaggcccacggtctgacccctgaacaggttgtggccatcgcctccaacatcggcggaaagcaggcactggaaacggtgcagagactgctccccgtgctatgccaagcgcatggactgactccggaacaagtagtcgccatcgccagcaatggtggagggaaacaagccctggagactgtgcagcgcctcctgccggtgctgtgtcaggcgcacggactcacccctgagcaagtggtcgccattgcctcgaacggtggcggcaagcaggctctggaaaccgtccagaggctgctcccggtgctctgccaagcccacggactgaccccagaacaggtggtcgcaattgcgtccaacggcggtggaaaacaagcgctggaaactgtccagcggctgctgccagtgctctgtcaagcgcacggcctgacacctgaacaagtcgtggcaatcgcgtcccacgatgggggaaaacaggccttggaaacggtccagcgcctgctgcccgtgttgtgtcaggctcatgggctgacccccgaacaagtggtggccattgccagccacgacggcggcaaacaggcccttgagaccgtgcagcggttgctgccagtgctttgccaagctcacgggctgaccccggagcaagtcgtcgcaatcgcctcccacgacgggggcaaacaggccctggaaaccgtgcagaggctgctccctgtgttgtgtcaggcccacggtctgactcctgaacaagtcgtggccatcgcttccaacggcggcggaaaacaggcactcgaaactgtgcagcgcctgctgcccgtgctgtgccaggctcatggactgacgccggaacaggttgtcgccatcgcctcgaacaacggtggaaaacaagccctggagactgtccagaggctccttcccgtgctctgccaagcacatggactcaccccggaacaagtggtggcaatcgcgtcccgccacggaggaaagcaggccttggaaacagtgcagcggctgctcccggtgctgtgtcaagcccacggactgaccccagaacaggtcgtggcgattgccagcaacggtggcgggaagcaggcccttgagactgtgcagagactcctgcccgtgttgtgccaggcgcatggcctcacccctgagcaagtggtcgctatcgcgagccacgatggcggcagacccgccctggagtcaattgtggcgcagctgtcccggcctgatccggccctggccgccctgaccaacgaccacctggtggccctcgcctgcctgggcgggcggcctgcgctggacgccgtgaagaagggactgccccacgccccggccctgatcaagagaaccaatcggcgcattcccgagcggacctcgcaccgcgtg

SEQ ID NO: 337 - skipped

SEQ ID NO: 338 - RNA, 288 nt, Mus sp.; identical to SEQ ID NO: 317
  source 1..288: mol_type = genomic RNA

SEQ ID NO: 339 - DNA, 18 bp, Mus sp.
  source 1..18: mol_type = genomic DNA
cagggaatttccctggtc

SEQ ID NO: 340 - protein, 27 aa, synthetic construct
PTSAGKLGSGEGRGSLLTCGDVEENPG

SEQ ID NO: 341 - DNA, 20 bp, synthetic construct
  source 1..20: mol_type = other DNA
gttagccctaaatcgaccct

SEQ ID NO: 342 - DNA, 20 bp, synthetic construct
  source 1..20: mol_type = other DNA
ggtgtgcagttagcacaact

SEQ ID NO: 343 - DNA, 20 bp, synthetic construct
  source 1..20: mol_type = other DNA
gaattgcagacatcagtctg

SEQ ID NO: 344 - DNA, 20 bp, synthetic construct
  source 1..20: mol_type = other DNA
aggggtcaccaagaagagtg

SEQ ID NO: 345 - DNA, 20 bp, synthetic construct
  source 1..20: mol_type = other DNA
ggtttctcattagcaatcag

SEQ ID NO: 346 - DNA, 20 bp, synthetic construct
  source 1..20: mol_type = other DNA
tccttgggaaattccacttg

SEQ ID NO: 347 - DNA, 20 bp, synthetic construct
  source 1..20: mol_type = other DNA
gtcataacccaggagtcacg

SEQ ID NO: 348 - DNA, 20 bp, synthetic construct
  source 1..20: mol_type = other DNA
gtttctgggctagagaactc

SEQ ID NO: 349 - DNA, 20 bp, synthetic construct
  source 1..20: mol_type = other DNA
aatggaacaagtcagcatga

SEQ ID NO: 350 - DNA, 20 bp, synthetic construct
  source 1..20: mol_type = other DNA
ctggcaagggtgagacagag

SEQ ID NO: 351 - DNA, 20 bp, synthetic construct
  source 1..20: mol_type = other DNA
catggtaaacaacagtacac

SEQ ID NO: 352 - DNA, 20 bp, synthetic construct
  source 1..20: mol_type = other DNA
gttgtggatatatccaggca

SEQ ID NO: 353 - DNA, 20 bp, synthetic construct
  source 1..20: mol_type = other DNA
tacgcatgaatgagatcact

SEQ ID NO: 354 - DNA, 20 bp, synthetic construct
  source 1..20: mol_type = other DNA
ttgtttttaatgcttgcatg

SEQ ID NO: 355 - DNA, 20 bp, synthetic construct
  source 1..20: mol_type = other DNA
tcattccgtgacatccagga

SEQ ID NO: 356 - DNA, 20 bp, synthetic construct
  source 1..20: mol_type = other DNA
cctccgtgactaagccaagg

SEQ ID NO: 357 - DNA, 20 bp, synthetic construct
  source 1..20: mol_type = other DNA
cccctactctcagcctgcat

SEQ ID NO: 358 - DNA, 20 bp, synthetic construct
  source 1..20: mol_type = other DNA
actccccctctgttttacac

SEQ ID NO: 359 - DNA, 20 bp, synthetic construct
  source 1..20: mol_type = other DNA
ctgagtttgtgtggagtatg

SEQ ID NO: 360 - DNA, 20 bp, synthetic construct
  source 1..20: mol_type = other DNA
tttagactactgtctgactg

SEQ ID NO: 361 - DNA, 20 bp, synthetic construct
  source 1..20: mol_type = other DNA
agctgtggcagtgatgcgag

SEQ ID NO: 362 - DNA, 20 bp, synthetic construct
  source 1..20: mol_type = other DNA
aacaggaaatattcctccca

SEQ ID NO: 363 - DNA, 20 bp, synthetic construct
  source 1..20: mol_type = other DNA
aggatcttctggaagtgtca

SEQ ID NO: 364 - DNA, 100 bp, synthetic construct
  source 1..100: mol_type = other DNA
gttagccctaaatcgaccctgttttagagctagaaatagcaagttaaaataaggctagtccgttatcaacttgaaaaagtggcaccgagtcggtgctttt

SEQ ID NO: 365 - DNA, 100 bp, synthetic construct
  source 1..100: mol_type = other DNA
ggtgtgcagttagcacaactgttttagagctagaaatagcaagttaaaataaggctagtccgttatcaacttgaaaaagtggcaccgagtcggtgctttt

SEQ ID NO: 366 - DNA, 100 bp, synthetic construct
  source 1..100: mol_type = other DNA
gaattgcagacatcagtctggttttagagctagaaatagcaagttaaaataaggctagtccgttatcaacttgaaaaagtggcaccgagtcggtgctttt

SEQ ID NO: 367 - DNA, 100 bp, synthetic construct
  source 1..100: mol_type = other DNA
aggggtcaccaagaagagtggttttagagctagaaatagcaagttaaaataaggctagtccgttatcaacttgaaaaagtggcaccgagtcggtgctttt

SEQ ID NO: 368 - DNA, 100 bp, synthetic construct
  source 1..100: mol_type = other DNA
ggtttctcattagcaatcaggttttagagctagaaatagcaagttaaaataaggctagtccgttatcaacttgaaaaagtggcaccgagtcggtgctttt

SEQ ID NO: 369 - DNA, 100 bp, synthetic construct
  source 1..100: mol_type = other DNA
tccttgggaaattccacttggttttagagctagaaatagcaagttaaaataaggctagtccgttatcaacttgaaaaagtggcaccgagtcggtgctttt

SEQ ID NO: 370 - DNA, 100 bp, synthetic construct
  source 1..100: mol_type = other DNA
gtcataacccaggagtcacggttttagagctagaaatagcaagttaaaataaggctagtccgttatcaacttgaaaaagtggcaccgagtcggtgctttt

SEQ ID NO: 371 - DNA, 100 bp, synthetic construct
  source 1..100: mol_type = other DNA
gtttctgggctagagaactcgttttagagctagaaatagcaagttaaaataaggctagtccgttatcaacttgaaaaagtggcaccgagtcggtgctttt

SEQ ID NO: 372 - DNA, 100 bp, synthetic construct
  source 1..100: mol_type = other DNA
aatggaacaagtcagcatgagttttagagctagaaatagcaagttaaaataaggctagtccgttatcaacttgaaaaagtggcaccgagtcggtgctttt

SEQ ID NO: 373 - DNA, 100 bp, synthetic construct
  source 1..100: mol_type = other DNA
ctggcaagggtgagacagaggttttagagctagaaatagcaagttaaaataaggctagtccgttatcaacttgaaaaagtggcaccgagtcggtgctttt

SEQ ID NO: 374 - DNA, 100 bp, synthetic construct
  source 1..100: mol_type = other DNA
catggtaaacaacagtacacgttttagagctagaaatagcaagttaaaataaggctagtccgttatcaacttgaaaaagtggcaccgagtcggtgctttt

SEQ ID NO: 375 - DNA, 100 bp, synthetic construct
  source 1..100: mol_type = other DNA
gttgtggatatatccaggcagttttagagctagaaatagcaagttaaaataaggctagtccgttatcaacttgaaaaagtggcaccgagtcggtgctttt

SEQ ID NO: 376 - DNA, 100 bp, synthetic construct
  source 1..100: mol_type = other DNA
tacgcatgaatgagatcactgttttagagctagaaatagcaagttaaaataaggctagtccgttatcaacttgaaaaagtggcaccgagtcggtgctttt

SEQ ID NO: 377 - DNA, 100 bp, synthetic construct
  source 1..100: mol_type = other DNA
ttgtttttaatgcttgcatggttttagagctagaaatagcaagttaaaataaggctagtccgttatcaacttgaaaaagtggcaccgagtcggtgctttt

SEQ ID NO: 378 - DNA, 100 bp, synthetic construct
  source 1..100: mol_type = other DNA
tcattccgtgacatccaggagttttagagctagaaatagcaagttaaaataaggctagtccgttatcaacttgaaaaagtggcaccgagtcggtgctttt

SEQ ID NO: 379 - DNA, 100 bp, synthetic construct
  source 1..100: mol_type = other DNA
cctccgtgactaagccaagggttttagagctagaaatagcaagttaaaataaggctagtccgttatcaacttgaaaaagtggcaccgagtcggtgctttt

SEQ ID NO: 380 - DNA, 100 bp, synthetic construct
  source 1..100: mol_type = other DNA
cccctactctcagcctgcatgttttagagctagaaatagcaagttaaaataaggctagtccgttatcaacttgaaaaagtggcaccgagtcggtgctttt

SEQ ID NO: 381 - DNA, 100 bp, synthetic construct
  source 1..100: mol_type = other DNA
actccccctctgttttacacgttttagagctagaaatagcaagttaaaataaggctagtccgttatcaacttgaaaaagtggcaccgagtcggtgctttt

SEQ ID NO: 382 - DNA, 100 bp, synthetic construct
  source 1..100: mol_type = other DNA
ctgagtttgtgtggagtatggttttagagctagaaatagcaagttaaaataaggctagtccgttatcaacttgaaaaagtggcaccgagtcggtgctttt

SEQ ID NO: 383 - DNA, 100 bp, synthetic construct
  source 1..100: mol_type = other DNA
tttagactactgtctgactggttttagagctagaaatagcaagttaaaataaggctagtccgttatcaacttgaaaaagtggcaccgagtcggtgctttt

SEQ ID NO: 384 - DNA, 100 bp, synthetic construct
  source 1..100: mol_type = other DNA
agctgtggcagtgatgcgaggttttagagctagaaatagcaagttaaaataaggctagtccgttatcaacttgaaaaagtggcaccgagtcggtgctttt

SEQ ID NO: 385 - DNA, 100 bp, synthetic construct
  source 1..100: mol_type = other DNA
aacaggaaatattcctcccagttttagagctagaaatagcaagttaaaataaggctagtccgttatcaacttgaaaaagtggcaccgagtcggtgctttt

SEQ ID NO: 386 - DNA, 100 bp, synthetic construct
  source 1..100: mol_type = other DNA
aggatcttctggaagtgtcagttttagagctagaaatagcaagttaaaataaggctagtccgttatcaacttgaaaaagtggcaccgagtcggtgctttt